(19) United States
(12) Patent Application Publication
(10) Pub. No.: US 2023/0113037 A1
(43) Pub. Date: Apr. 13, 2023

(54) [1,2,4]TRIAZOLO[1,5-C]QUINAZOLIN-5-AMINES

(71) Applicants: Bayer Aktiengesellschaft, Leverkusen (DE); Bayer Pharma Aktiengesellschaft, Berlin (DE); DEUTSCHES KREBSFORSCHUNGSZENTRUM, Heidelberg (DE)

(72) Inventors: Julien LEFRANC, Darmstadt (DE); Norbert SCHMEES, Berlin (DE); Ludwig ZORN, Berlin (DE); Robin Michael MEIER, Wain (DE); Simon Anthony HERBERT, Berlin (DE); Judith GÜNTHER, Berlin (DE); Ilona GUTCHER, Berlin (DE); Lars RÖSE, Berlin (DE); Benjamin BADER, Berlin (DE); Detlef STÖCKIGT, Potsdam (DE); Mátyás GORJÁNÁCZ, Berlin (DE); Christina KOBER, Lollar (DE); Bernd BUCHMANN, Hohen Neuendorf (DE); Stephan BÖHME, Berlin (DE); Ulrich BOTHE, Berlin (DE); Michael PLATTEN, Heidelberg (DE); Daniel BAUMANN, Heidelberg (DE)

(73) Assignees: Bayer Aktiengesellschaft, Leverkusen (DE); Bayer Pharma Aktiengesellschaft, Berlin (DE); DEUTSCHES KREBSFORSCHUNGSZENTRUM, Heidelberg (DE)

(21) Appl. No.: 17/634,930

(22) PCT Filed: Aug. 10, 2020
(86) PCT No.: PCT/EP2020/072377

§ 371 Date: Feb. 11, 2022

(30) Foreign Application Priority Data
Aug. 12, 2019 (EP) 19191299.7
Apr. 2, 2020 (EP) 20167707.7

(51) Int. Cl. C07D 487/04

(52) U.S. Cl. CPC C07D 487/04

(57) ABSTRACT

The present invention covers [1,2,4]triazolo[1,5-c]quinazolin-5-amine compounds of general formula (I) in which R¹, R², R³, R⁴, R⁵, R⁶, R⁷ and R⁸ are as defined herein, methods of preparing said compounds, intermediate compounds useful for preparing said compounds, pharmaceutical compositions and combinations comprising said compounds and the use of said compounds for manufacturing pharmaceutical compositions for the treatment or prophylaxis of diseases, in particular of cancer or conditions with dysregulated immune responses or other disorders associated with aberrant AHR signaling, as a sole agent or in combination with other active ingredients.

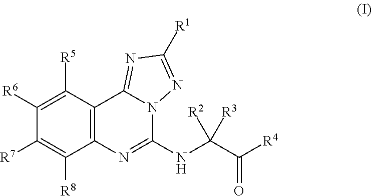

The present invention covers [1,2,4]triazolo[1,5-c]quinazolin-5-amine compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular cancer or conditions with dysregulated immune responses, as a sole agent or in combination with other active ingredients.

### BACKGROUND

The AHR (Aryl Hydrocarbon Receptor) is a ligand-activated transcription factor, belonging to the basic helix-loop-helix/Per-Arnt-Sim (bHLH/PAS) family, and is located in the cytosol. Upon ligand binding, the AHR translocates to the nucleus where it heterodimerises with ARNT (AHR Nuclear Translocator) upon which it interacts with DREs (Dioxin Response Elements) of AHR-responsive genes to regulate their transcription. The AHR is best known for binding to environmental toxins and inducing the metabolic machinery, such as cytochrome P 450 enzymes (eg. CYP1A1, CYP1A2 and CYP1B1), required for their elimination (Reyes et al., Science, 1992, 256(5060):1193-5). Activation of AHR by xenobiotics has demonstrated its role in numerous cellular processes such as embryogenesis, tumourigenesis and inflammation.

AHR is expressed in many cells of the immune system, including dendritic cells (DCs), macrophages, T cells and NK cells, and plays an important role in immunoregulation (Nguyen et al., Front Immunol, 2014, 5:551). The classic exogenous AHR ligands TCDD and 3-methylcholanthrene, for example, are known to induce profound immunosuppression, promote carcinogenesis and induce tumour growth (Gramatzki et al., Oncogene, 2009, 28(28):2593-605; Bui et al., Oncogene, 2009, 28(41):3642-51; Esser et al., Trends Immunol, 2009, 30:447-454). In the context of immunosuppression, AHR activation promotes regulatory T cell generation, inhibits Th1 and Th17 differentiation, directly and indirectly, and decreases the activation and maturation of DCs (Wang et al., Clin Exp Immunol, 2014, 177(2):521-30; Mezrich et al., J Immunol, 2010,185(6): 3190-8; Wei et al., Lab Invest, 2014, 94(5):528-35; Nguyen et al., PNAS, 2010, 107(46):19961-6). AHR activation modulates the innate immune response and constitutive AHR expression has been shown to negatively regulate the type-I interferon response to viral infection (Yamada et al., Nat Immunol, 2016). Additionally, mice with a constitutively active AHR spontaneously develop tumours (Andersson et al., PNAS, 2002, 99(15):9990-5).

In addition to xenobiotics, the AHR can also bind metabolic products of tryptophan degradation. Tryptophan metabolites, such as kynurenine and kynurenic acid, are endogenous AHR ligands that activate the AHR under physiological conditions (DiNatale et al., Toxicol Sci, 2010, 115(1):89-97; Mezrich et al., J Immunol, 2010, 185(6):3190-8; Opitz et al., Nature, 2011, 478(7368):197-203). Other endogenous ligands are known to bind the AHR although their physiological roles are currently unknown (Nguyen & Bradfield, Chem Res Toxicol, 2008, 21(1):102-116).

The immunosuppressive properties of kynurenine and tryptophan degradation are well described and are implicated in cancer-associated immunosuppression. The enzymes indoleamine-2,3-dioxygenases 1 and 2 (IDO1/IDO2) as well as tryptophan-2,3-dioxygenase 2 (TDO2) are responsible for catalysing the first and rate-limiting step of tryptophan metabolism. IDO1/2-mediated degradation of tryptophan in tumours and tumour-draining lymph nodes reduces anti-tumour immune responses and inhibition of IDO can suppress tumour formation in animal models (Uyttenhove et al., Nat Med, 2003, 9(10):1269-74; Liu et al., Blood, 2005, 115(17): 3520-30; Muller et al., Nat Med, 11(3):312-9; Metz, Cancer Res, 2007, 67(15):7082-7). TDO2 is also strongly expressed in cancer and can lead to the production of immunosuppressive kynurenine. In glioma, activation of the AHR by kynurenine, downstream of TDO-mediated tryptophan degradation, enhances tumour growth as a consequence of inhibiting anti-tumour immune responses as well as directly promoting tumour cell survival and motility (Opitz et al., Nature, 2011, 478(7368):197-203). AHR ligands generated by tumour cells therefore act in both an autocrine and paracrine fashion on tumour cells and lymphocytes, respectively, to promote tumour growth.

The present invention covers [1,2,4]triazolo[1,5-c]quinazolin-5-amine compounds of general formula (I) which inhibit the AHR.

### STATE OF THE ART

WO 2010/059401 relates to compounds and compositions for expanding the number of CD34+ cells for transplantation. In particular, WO 2010/059401 relates inter alia to heterocyclic compounds capable of down-regulating the activity and/or expression of AHR.

WO 2012/015914 relates to compositions and methods for modulating AHR activity. In particular, WO 2012/015914 relates inter alia to heterocyclic compounds that modulate AHR activity for use in therapeutic compositions.

WO 2007040565 relates to the use of [1,2,4]triazolo[1,5-c]pyrimidin-5-amine derivatives as adenosine receptor antagonists.

U.S. Pat. No. 6,358,964 relates to [1,2,4]triazolo[1,5-c]quinazolin-5-amine derivatives useful as potent modulators of the adenosine A₃ receptor.

However, the state of the art does not describe the [1,2,4]triazolo[1,5-c]quinazolin-5-amine compounds of general formula (I) of the present invention as described and defined herein.

It has now been found, and this constitutes the basis of the present invention, that the compounds of the present invention have surprising and advantageous properties.

In particular, the compounds of the present invention have surprisingly been found to effectively inhibit AHR for which data are given in biological experimental section and may therefore be used for the treatment or prophylaxis of cancer or other conditions where exogenous and endogenous AHR ligands induce dysregulated immune responses, uncontrolled cell growth, proliferation and/or survival of tumour cells, immunosuppression in the context of cancer, inappropriate cellular immune responses, or inappropriate cellular inflammatory responses or diseases which are accompanied with uncontrolled cell growth, proliferation and/or survival of tumour cells, immunosuppression in the context of cancer inappropriate cellular immune responses, or inappropriate cellular inflammatory responses, particularly in which the uncontrolled cell growth, proliferation and/or survival of tumour cells, immunosuppression in the context of cancer, inappropriate cellular immune responses, or inappropriate cellular inflammatory responses is mediated by AHR, such as, for example, liquid and solid tumours, and/or metastases thereof, e.g. head and neck tumours including brain tumours and brain metastases, tumours of the thorax including non-small cell and small cell lung tumours, gastrointestinal tumours including colon, colorectal and pancreatic tumours, liver tumours, endocrine tumours, mammary and other gynecological tumours, urological tumours including renal, bladder and prostate tumours, skin tumours, and sarcomas, and/or metastases thereof.

### DESCRIPTION OF THE INVENTION

In accordance with a first aspect, the present invention covers compounds of general formula (I):

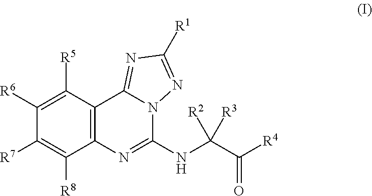

in which
R¹ represents phenyl or heteroaryl,
   optionally substituted one to three times, independently from each other, with halogen, cyano, hydroxy, C₁-C₄-alkyl, C₁-C₄-alkoxy, C₁-C₄-haloalkyl, C₁-C₄-haloalkoxy, C₁-C₄-hydroxyalkyl, C₁-C₄-alkoxy-C₁-C₄-alkyl-, C₃-C₆-cycloalkyl, C₃-C₆-cycloalkyl-C₁-C₄-alkyl-, C₃-C₆-cycloalkyl-O—, 4- to 6-membered heterocycloalkyl, —NR⁹R¹⁰, R⁹R¹⁰N—C₁-C₄-alkyl-, C₁-C₃-alkyl-S(O)ₘ— or C₁-C₃-alkyl-SO(NH)—;
R² represents hydrogen, C₁-C₄-alkyl, C₁-C₄-haloalkyl or C₃-C₆-cycloalkyl;
R³ represents hydrogen, C₁-C₆-alkyl, phenyl or phenyl-C₁-C₃-alkyl, wherein said C₁-C₆-alkyl group is optionally substituted, one or more times, independently from each other, with hydroxy, halogen, C₁-C₄-alkoxy, —S(O)ₙ—C₁-C₄-alkyl, phenyl-C₁-C₃-alkoxy or —NR⁹R¹⁰ and
   said phenyl groups are optionally substituted, one or more times, independently from each other, with hydroxy, halogen, cyano, C₁-C₃-alkyl, C₁-C₃-haloalkyl, C₁-C₃-alkoxy or C₁-C₃-haloalkoxy, or
R² and R³ together with the carbon atom to which they are attached form a 3- to 6-membered ring, said ring optionally containing one heteroatom selected from O, S, NH, NR^{a} in which R^{a} represents a C₁-C₄-alkyl group;
R⁴ represents hydroxy, C₁-C₄-alkoxy or —NR¹¹R¹², or
R² and R⁴ together represent *—C₂-C₅-alkanediyl-X¹—**, *—C₁-C₂-alkanediyl-X²—C₁-C₃-alkanediyl-** or *—C₁-C₂-alkanediyl-X²—C₂-C₃-alkanediyl-X¹—** to form a 5- to 9-membered ring,
   wherein * indicates the point of attachment of said group for R² and ** indicates the point of attachment of said group for R⁴;
R⁵ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 4- to 6-membered heterocycloalkyl, —CO₂—C₁-C₄-alkyl, —CO—NR⁹R¹⁰ or —NR⁹R¹⁰;
R⁶ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁷ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁸ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 1-R¹⁵—C₃-C₆-cycloalkyl, —CO₂—C₁-C₄-alkyl, —CO—NR⁹R¹⁰, —NR⁹R¹⁰, C₁-C₄-hydroxyalkyl, C₁-C₄-alkoxy-C₁-C₄-alkyl-, C₁-C₄-alkyl-S—, C₁-C₄-alkyl-S—C₁-C₄-alkyl-, —S(═O)R′, —S(═O)₂R′, —S(═O)₂NH₂, —S(═O)₂NHR′, —S(═O)₂N(R′)R″, —S(═O)(═NH)R′, 4- to 6-membered heterocycloalkyl, or —OR¹⁶;
R⁹ and R¹⁰ are the same or different and represent, independently from each other, hydrogen, C₁-C₃-alkyl or tert-butoxycarbonyl, or
   together with the nitrogen atom to which they are attached form a 4- to 6-membered nitrogen containing heterocyclic ring, said ring optionally containing one additional heteroatom selected from O, S, NH, NR^{a} in which R^{a} represents C₁-C₄-alkyl or C₁-C₄-alkoxycarbonyl;
R¹¹ and R¹² are the same or different and represent, independently from each other, hydrogen, C₁-C₄-alkyl, C₂-C₄-hydroxyalkyl, C₁-C₄-alkoxy-C₂-C₄-alkyl-, R⁹R¹⁰N—C₂-C₄-alkyl-, C₃-C₆-cycloalkyl, 4- to 7-membered heterocycloalkyl, said 4- to 7-membered heterocycloalkyl group is optionally substituted, one or two times, independently from each other, with hydroxy, oxo, halogen, C₁-C₄-alkyl, C₁-C₄-alkoxy, or —NR⁹R¹⁰, or
   together with the nitrogen atom to which they are attached form a 4- to 6-membered nitrogen containing heterocyclic ring, said ring optionally containing one additional heteroatom selected from O, S, NH, NR^{a} in which R^{a} represents C₁-C₄-alkyl or C₁-C₄-alkoxycarbonyl and is optionally substituted, one or two times, independently from each other, with hydroxy, halogen, C₁-C₄-alkyl, C₁-C₄-alkoxy, or —NR⁹R¹⁰, or
   together with the nitrogen atom to which they are attached form a heterospirocycloalkyl group, which is optionally substituted, one or two times, independently from each other, with hydroxy, halogen, C₁-C₄-alkyl, C₁-C₄-alkoxy, or —NR⁹R¹⁰, or
   together with the nitrogen atom to which they are attached form a bridged heterocycloalkyl group, which is optionally substituted, one or two times, independently from each other, with hydroxy, halogen, C₁-C₄-alkyl, C₁-C₄-alkoxy, or —NR⁹R¹⁰;
R¹³ represents hydrogen, C₁-C₄-alkyl, benzyl, 4-methoxybenzyl or tert-butoxycarbonyl;
R¹⁴ represents hydrogen, C₁-C₄-alkyl, benzyl or 4-methoxybenzyl;
R¹⁵ represents C₁-C₃-alkyl or C₁-C₃-haloalkyl;
R¹⁶ represents C₂-C₆-hydroxyalkyl, C₁-C₄-alkoxy-C₂-C₆-alkyl-, or C₃-C₆-cycloalkyl;
R′ and R″ represent, independently from each other, C₁-C₆-alkyl, C₁-C₆-haloalkyl, or C₃-C₆-cycloalkyl;
X¹ represents O, S(O)ₘ, or NR¹³;
X² represents O, S(O)ₘ, or NR¹⁴;
m represents 0, 1 or 2;
n represents 0, 1 or 2;
   their polymorphs, enantiomeres, diastereomeres, racemates, tautomeres, N-oxides, hydrates and solvates, as well as their physiological acceptable salts and solvates of these salts, as well as mixtures of the same.

Further, it covers their use in combination with other anti cancer medications such as immunotherapeutics, targeted anti cancer agents or chemotherapy.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon atom. Commonly, it is possible for the number of optional substituents, when present, to be 1, 2 or 3.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen” means a fluorine, chlorine, bromine or iodine, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₄-hydroxyalkyl” means a linear or branched, saturated, monovalent hydrocarbon group in which the term “C₁-C₄-alkyl” is defined supra, and in which 1 or 2 hydrogen atoms are replaced with a hydroxy group, e.g. a hydroxymethyl, 1-hydroxyethyl, 2-hydroxyethyl, 1,2-di-hydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl or 1-hydroxy-2-methyl-propyl group.

The term “C₁-C₄-alkoxy” means a linear or branched, saturated, monovalent group of formula (C₁-C₄-alkyl)-O—, which means methoxy, ethoxy, n-propoxy, isopropoxy, n-butoxy, sec-butoxy, isobutoxy or tert-butoxy.

The term “C₁-C₄-haloalkoxy” means a linear or branched, saturated, monovalent C₁-C₄-alkoxy group, as defined supra, in which one or more of the hydrogen atoms is replaced, identically or differently, with a halogen atom. Particularly, said halogen atom is a fluorine atom. Said C₁-C₄-haloalkoxy group is, for example, fluoromethoxy, difluoromethoxy, trifluoromethoxy, 2,2,2-trifluoroethoxy or pentafluoroethoxy.

The term “C₁-C₅-alkanediyl” means a bivalent saturated aliphatic radical regarded as derived from an C₁-C₅-alkane by removal of a hydrogen atom from each of the two terminal carbon atoms of the chain, e.g. a methylene, ethylene, propylene, trimethylene, tetramethylene or pentamethylene.

The term “C₃-C₆-cycloalkyl” means a saturated, monovalent, monocyclic hydrocarbon ring which contains 3, 4, 5 or 6 carbon atoms (“C₃-C₆-cycloalkyl”). Said C₃-C₆-cycloalkyl group is a monocyclic hydrocarbon ring, e.g. a cyclopropyl, cyclobutyl, cyclopentyl or cyclohexyl.

The term “4- to 7-membered heterocycloalkyl” means a monocyclic, saturated heterocycle with 4, 5, 6 or 7 ring atoms in total, which contains one or two identical or different heteroatom-containing groups selected from the group consisting of —NR^{b}—, —O—, —S—, —SO—, —SO₂—, —SO₂—NR^{b}—, —SO(═NR^{b})—, wherein R^{b} means a hydrogen atom or a C₁-C₃-alkyl group. It being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, tetrahydrothiophene 1-oxide, 1,2-thiazolidine 1-oxide, 1,3-thiazolidine 1-oxide, tetrahydrothiophene 1,1-dioxide, 1,2-thiazolidine 1,1-dioxide, 1,3-thiazolidine 1,1-dioxide, 1,2,5-thiadiazolidine 1,1-dioxide, 1,2,4-thiadiazolidine 1,1-dioxide, 1,2,3-thiadiazolidine 1,1-dioxide, tetrahydro-1H-1λ⁴-thiophen-1-imine 1-oxide, 1λ⁴,2-thiazolidin-1-imine 1-oxide or 1λ⁴,3-thiazolidin-1-imine 1-oxide, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, tetrahydro-2H-thiopyran 1-oxide, 1,2-thiazinane 1-oxide, 1,3-thiazinane 1-oxide, thiomorpholine 1-oxide, tetrahydro-2H-thiopyran 1,1-dioxide, 1,2-thiazinane 1,1-dioxide, 1,3-thiazinane 1,1-dioxide, thiomorpholine 1,1-dioxide, 1,2,6-thiadiazinane 1,1-dioxide, 1,2,5-thiadiazinane 1,1-dioxide, 1,2,4-thiadiazinane 1,1-dioxide, 1,2,3-thiadiazinane 1,1-dioxide, hexahydro-1λ⁴-thiopyran-1-imine 1-oxide, 1λ⁴,2-thiazinan-1-imine 1-oxide, 1λ⁴,3-thiazinan-1-imine 1-oxide or 1λ⁴-thiomorpholin-1-imine 1-oxide, or a 7-membered ring, such as azepanyl, 1,4-diazepanyl, 1,4-oxazepanyl, 1,4-thiazepanyl, or 1-imino-1λ⁶,4-thiazepane-1-oxid, for example.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro[5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “bridged heterocycloalkyl” means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo-[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]-nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

The term “monocyclic heteroaryl” means a monovalent, aromatic ring having 5 or 6 ring atoms (a “5- or 6-membered heteroaryl” group), which contains at least one ring heteroatom and optionally one or two further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Further, it is possible for compounds of the present invention to exist as tautomers. For example, any compound which contains a [1,2,4]triazolo[1,5-c]quinazolin-5(6H)-one moiety for example can exist as a keto tautomer, or an enol tautomer, or even a mixture in any amount of the two tautomers, namely:

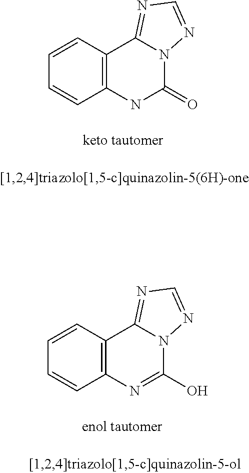 [SMILES: O=c1[nH]c2ccccc2c2ncnn12 ; Oc1nc2ccccc2c2ncnn12]

The present invention includes all possible tautomers of the compounds of the present invention as single tautomers, or as any mixture of said tautomers, in any ratio.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown. Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

The invention further includes all possible crystallized and polymorphic forms of the inventive compounds, whereby the polymorphs are existing either as a single polymorph form or are existing as a mixture of several polymorphs in all concentrations.

The compounds are either commercially available or can be prepared according to procedures available from the public domain, as understandable to the person skilled in the art. Specific examples are described in the Experimental Section.

In accordance with a second embodiment of the first aspect, the present invention covers compounds of general formula (I), supra, in which:
R¹ represents phenyl or heteroaryl,
   optionally substituted one to three times, independently from each other, with halogen, cyano, hydroxy, C₁-C₄-alkyl, C₁-C₄-alkoxy, C₁-C₄-haloalkyl, C₁-C₄-haloalkoxy, C₃-C₆-cycloalkyl, C₃-C₆-cycloalkyl-C₁-C₄-alkyl-, C₃-C₆-cycloalkyl-O—, 4- to 6-membered heterocycloalkyl, —NR⁹R¹⁰ or R⁹R¹⁰N—C₁-C₄-alkyl;
R² represents hydrogen, C₁-C₄-alkyl, C₁-C₄-haloalkyl or C₃-C₆-cycloalkyl;
R³ represents hydrogen, C₁-C₆-alkyl, phenyl or phenyl-C₁-C₃-alkyl, wherein
   said C₁-C₆-alkyl group is optionally substituted, one or more times, independently from each other, with hydroxy, halogen, C₁-C₄-alkoxy, —S(O)ₙ—C₁-C₄-alkyl, phenyl-C₁-C₃-alkoxy or —NR⁹R¹⁰ and
   said phenyl groups are optionally substituted, one or more times, independently from each other, with hydroxy, halogen, cyano, C₁-C₃-alkyl, C₁-C₃-haloalkyl, C₁-C₃-alkoxy or C₁-C₃-haloalkoxy, or
R² and R³ together with the carbon atom to which they are attached form a 3- to 6-membered ring, said ring optionally containing one heteroatom selected from O, S, NH, NR^{a} in which R^{a} represents a C₁-C₄-alkyl group;
R⁴ represents hydroxy, C₁-C₄-alkoxy or —NR¹¹R¹², or
R² and R⁴ together represent *—C₂-C₅-alkanediyl-X¹—**, *—C₁-C₂-alkanediyl-X²—C₁-C₃-alkanediyl-** or *—C₁-C₂-alkanediyl-X²—C₂-C₃-alkanediyl-X¹—** to form a 5- to 9-membered ring,
   wherein * indicates the point of attachment of said group for R² and ** indicates the point of attachment of said group for R⁴;
R⁵ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁶ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁷ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁸ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁹ and R¹⁰ are the same or different and represent, independently from each other, hydrogen, C₁-C₃-alkyl or tert-butoxycarbonyl, or
   together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group;
R¹¹ and R¹² are the same or different and represent, independently from each other, hydrogen, C₁-C₄-alkyl or C₃-C₆-cycloalkyl, wherein said C₁-C₄-alkyl group is optionally substituted with hydroxy;
R¹³ represents hydrogen, C₁-C₄-alkyl, benzyl, 4-methoxybenzyl or tert-butoxycarbonyl;
R¹⁴ represents hydrogen, C₁-C₄-alkyl, benzyl or 4-methoxybenzyl;
X¹ represents O or NR¹³;
X² represents O or NR¹⁴;
n represents 0, 1 or 2;
   their polymorphs, enantiomeres, diastereomeres, racemates, tautomeres, N-oxides, hydrates and solvates, as well as their physiological acceptable salts and solvates of these salts, as well as mixtures of the same.

In accordance with a third embodiment of the first aspect, the present invention covers compounds of general formula (I), supra, in which:
R¹ represents phenyl or monocyclic heteroaryl,
   optionally substituted one to two times, independently from each other, with halogen, hydroxy, C₁-C₄-alkyl, C₁-C₄-alkoxy, C₁-C₄-haloalkyl, C₁-C₄-haloalkoxy, C₃-C₆-cycloalkyl, C₃-C₆-cycloalkyl-C₁-C₄-alkyl-, C₃-C₆-cycloalkyl-O—, 4- to 6-membered heterocycloalkyl or —NR⁹R¹⁰;
R² represents hydrogen or C₁-C₄-alkyl;
R³ represents hydrogen, C₁-C₄-alkyl, phenyl or phenyl-methyl, wherein
   said C₁-C₄-alkyl group is optionally substituted once with hydroxy, methoxy, —S(O)ₙ-methyl, phenyl-methoxy or —NR⁹R¹⁰ and
   said phenyl groups are optionally substituted once with hydroxy, or
R² and R³ together with the carbon atom to which they are attached form a 3- to 6-membered ring, said ring optionally containing one oxygen atom;
R⁴ represents hydroxy, methoxy or —NR¹¹R¹², or
R² and R⁴ together represent a group selected from:

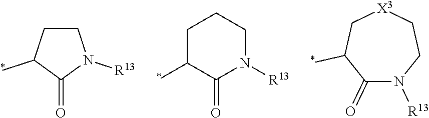

wherein * indicates the point of attachment of said group with the NH group in formula (I);
R⁵ represents hydrogen, halogen, C₁-C₄-alkyl, methoxy, trifluoromethyl or cyclopropyl;
R⁶ represents hydrogen, halogen or methyl;
R⁷ represents hydrogen, halogen, methyl or methoxy;
R⁸ represents hydrogen, halogen or methyl;
R⁹ and R¹⁰ are the same or different and represent, independently from each other, hydrogen, methyl or tert-butoxycarbonyl;
R¹¹ and R¹² are the same or different and represent, independently from each other, hydrogen, C₁-C₃-alkyl or C₃-C₄-cycloalkyl, wherein said C₁-C₃-alkyl group is optionally substituted with hydroxy;
R¹³ represents hydrogen or methyl;
X³ represents CH₂ or NH;
n represents O or 2;
   their polymorphs, enantiomeres, diastereomeres, racemates, tautomeres, N-oxides, hydrates and solvates, as well as their physiological acceptable salts and solvates of these salts, as well as mixtures of the same.

In accordance with a forth embodiment of the first aspect, the present invention covers compounds of general formula (Ia):

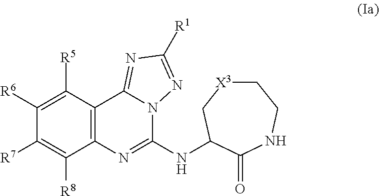

in which:
R¹ represents phenyl or heteroaryl,
   optionally substituted one to three times, independently from each other, with halogen, cyano, hydroxy, C₁-C₄-alkyl, C₁-C₄-alkoxy, C₁-C₄-haloalkyl, C₁-C₄-haloalkoxy, C₃-C₆-cycloalkyl, C₃-C₆-cycloalkyl-C₁-C₄-alkyl-, C₃-C₆-cycloalkyl-O—, 4- to 6-membered heterocycloalkyl, —NR⁹R¹⁰ or R⁹R¹⁰N—C₁-C₄-alkyl;
R⁵ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁶ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁷ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁸ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁹ and R¹⁰ are the same or different and represent, independently from each other, hydrogen, C₁-C₃-alkyl or tert-butoxycarbonyl, or
   together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group;
R¹⁴ represents hydrogen, C₁-C₄-alkyl, benzyl or 4-methoxybenzyl;
X³ represents CH₂ or NR¹⁴;
   their polymorphs, enantiomeres, diastereomeres, racemates, tautomeres, N-oxides, hydrates and solvates, as well as their physiological acceptable salts and solvates of these salts, as well as mixtures of the same.

In accordance with a fifth embodiment of the first aspect, the present invention covers compounds of general formula (Ib):

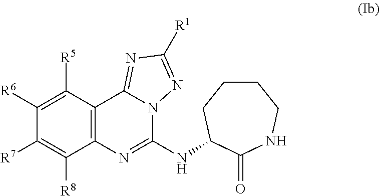

in which:
R¹ represents phenyl, pyridinyl, pyridazinyl, furanyl, oxazolyl, pyrazolyl or oxadiazolyl, optionally substituted once or twice, independently from each other, with fluoro, chloro, C₁-C₄-alkyl, methoxy, trifluoromethyl, trifluoromethoxy, cyclopropyl, oxanyl or —N(CH₃)₂;
R⁵, R⁶, R⁷, R³ represent, independently from each other, hydrogen, fluoro, chloro, bromo, methyl, methoxy, trifluoromethyl or cyclopropyl;
   their polymorphs, tautomeres, N-oxides, hydrates and solvates, as well as their physiological acceptable salts and solvates of these salts, as well as mixtures of the same.

In accordance with a sixth embodiment of the first aspect, the present invention covers compounds of general formula (I):

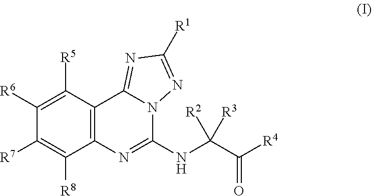

in which
R¹ represents phenyl or heteroaryl, optionally substituted one to three times, independently from each other, with halogen, cyano, hydroxy, C₁-C₄-alkyl, C₁-C₄-alkoxy, C₁-C₄-haloalkyl, C₁-C₄-haloalkoxy, C₁-C₄-hydroxyalkyl, C₁-C₄-alkoxy-C₁-C₄-alkyl-, C₃-C₆-cycloalkyl, C₃-C₆-cycloalkyl-C₁-C₄-alkyl-, C₃-C₆-cycloalkyl-O—, 4- to 6-membered heterocycloalkyl, —NR⁹R¹⁰, R⁹R¹⁰N—C₁-C₄-alkyl-, C₁-C₃-alkyl-S(O)ₘ— or C₁-C₃-alkyl-SO(NH)—;
R² represents hydrogen, C₁-C₄-alkyl, C₁-C₄-haloalkyl or C₃-C₆-cycloalkyl;
R³ represents hydrogen, C₁-C₆-alkyl, phenyl or phenyl-C₁-C₃-alkyl, wherein
   said C₁-C₆-alkyl group is optionally substituted, one or more times, independently from each other, with hydroxy, halogen, C₁-C₄-alkoxy, —S(O)ₙ—C₁-C₄-alkyl, phenyl-C₁-C₃-alkoxy or —NR⁹R¹⁰ and
   said phenyl groups are optionally substituted, one or more times, independently from each other, with hydroxy, halogen, cyano, C₁-C₃-alkyl, C₁-C₃-haloalkyl, C₁-C₃-alkoxy or C₁-C₃-haloalkoxy, or
R² and R³ together with the carbon atom to which they are attached form a 3- to 6-membered ring, said ring optionally containing one heteroatom selected from O, S, NH, NR^{a} in which R^{a} represents a C₁-C₄-alkyl group;
R⁴ represents hydroxy, C₁-C₄-alkoxy or —NR¹¹R¹², or
R² and R⁴ together represent *—C₂-C₅-alkanediyl-X¹—**, *—C₁-C₂-alkanediyl-X²—C₁-C₃-alkanediyl-** or *—C₁-C₂-alkanediyl-X²—C₂-C₃-alkanediyl-X¹—** to form a 5- to 9-membered ring,
   wherein * indicates the point of attachment of said group for R² and ** indicates the point of attachment of said group for R⁴;
R⁵ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 4- to 6-membered heterocycloalkyl, —CO₂—C₁-C₄-alkyl, —CO—NR⁹R¹⁰ or —NR⁹R¹⁰;
R⁶ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁷ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁸ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 1-R¹⁵—C₃-C₆-cycloalkyl, —CO₂—C₁-C₄-alkyl, —CO—NR⁹R¹⁰ or —NR⁹R¹⁰;
R⁹ and R¹⁰ are the same or different and represent, independently from each other, hydrogen, C₁-C₃-alkyl or tert-butoxycarbonyl, or
   together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group;
R¹¹ and R¹² are the same or different and represent, independently from each other, hydrogen, C₁-C₄-alkyl or C₃-C₆-cycloalkyl, wherein said C₁-C₄-alkyl group is optionally substituted with hydroxy;
R¹³ represents hydrogen, C₁-C₄-alkyl, benzyl, 4-methoxybenzyl or tert-butoxycarbonyl;
R¹⁴ represents hydrogen, C₁-C₄-alkyl, benzyl or 4-methoxybenzyl;
R¹⁵ represents C₁-C₃-alkyl or C₁-C₃-haloalkyl;
X¹ represents O or NR¹³;
X² represents O or NR¹⁴;
m represents 0, 1 or 2;
n represents 0, 1 or 2;
   their polymorphs, enantiomeres, diastereomeres, racemates, tautomeres, N-oxides, hydrates and solvates, as well as their physiological acceptable salts and solvates of these salts, as well as mixtures of the same.

In accordance with a second aspect, the present invention covers methods of preparing compounds of general formula (I) as defined supra, said methods comprising the step of allowing an intermediate compound of general formula (V):

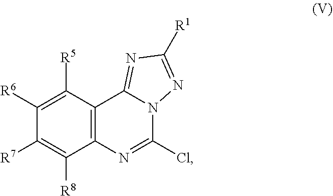

in which R¹, R⁵, R⁶, R⁷ and R⁸ are as defined supra to react with a compound of general formula (VII):

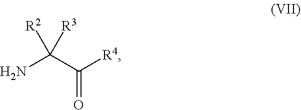

in which R², R³ and R⁴ are as defined supra thereby giving a compound of general formula (I):

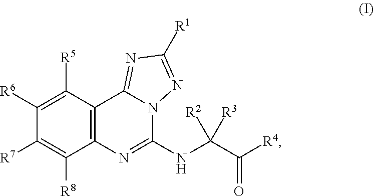

in which R¹, R², R³, R⁴, R⁵, R⁶, R⁷ and R⁸ are as defined supra.

The present invention covers methods of preparing compounds of the present invention of general formula (I), said methods comprising the steps as described in the Experimental Section herein.

In accordance with a third aspect, the present invention covers intermediate compounds which are useful for the preparation of the compounds of general formula (I), supra.

Particularly, the inventions covers the intermediate compounds of general formula (V):

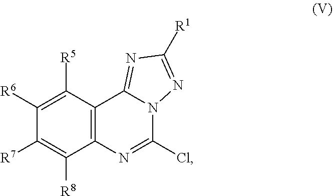

in which R¹, R⁵, R⁶, R⁷ and R⁸ are as defined supra.

In accordance with a forth aspect, the present invention covers the use of said intermediate compounds for the preparation of a compound of general formula (I) as defined supra.

Particularly, the inventions covers the use of intermediate compounds of general formula (V):

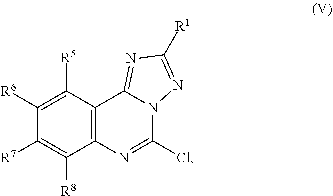

in which R¹, R⁵, R⁶, R⁷ and R³ are as defined supra for the preparation of a compound of general formula (I) as defined supra.

The present invention covers the intermediate compounds which are disclosed in the Example Section of this text, infra.

In a further embodiment of the first aspect, the present invention covers compounds of formula (I), supra, in which:
R¹ represents phenyl or heteroaryl,
   optionally substituted one to three times, independently from each other, with halogen, cyano, hydroxy, C₁-C₄-alkyl, C₁-C₄-alkoxy, C₁-C₄-haloalkyl, C₁-C₄-haloalkoxy, C₁-C₄-hydroxyalkyl, C₁-C₄-alkoxy-C₁-C₄-alkyl-, C₃-C₆-cycloalkyl, C₃-C₆-cycloalkyl-C₁-C₄-alkyl-, C₃-C₆-cycloalkyl-O—, 4- to 6-membered heterocycloalkyl, —NR⁹R¹⁰, R⁹R¹⁰N—C₁-C₄-alkyl-, C₁-C₃-alkyl-S(O)ₘ— or C₁-C₃-alkyl-SO(NH)—;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹ represents phenyl or heteroaryl,
   optionally substituted one to three times, independently from each other, with halogen, cyano, hydroxy, C₁-C₄-alkyl, C₁-C₄-alkoxy, C₁-C₄-haloalkyl, C₁-C₄-haloalkoxy, C₃-C₆-cycloalkyl, C₃-C₆-cycloalkyl-C₁-C₄-alkyl-, C₃-C₆-cycloalkyl-O—, 4- to 6-membered heterocycloalkyl, —NR⁹R¹⁰ or R⁹R¹⁰N—C₁-C₄-alkyl;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹ represents phenyl or monocyclic heteroaryl,
   optionally substituted one to three times, independently from each other, with halogen, cyano, hydroxy, C₁-C₄-alkyl, C₁-C₄-alkoxy, C₁-C₄-haloalkyl, C₁-C₄-haloalkoxy, C₃-C₆-cycloalkyl, C₃-C₆-cycloalkyl-C₁-C₄-alkyl-, C₃-C₆-cycloalkyl-O—, 4- to 6-membered heterocycloalkyl, —NR⁹R¹⁰ or R⁹R¹⁰N—C₁-C₄-alkyl;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹ represents phenyl or monocyclic heteroaryl,
   optionally substituted one to two times, independently from each other, with halogen, hydroxy, C₁-C₄-alkyl, C₁-C₄-alkoxy, C₁-C₄-haloalkyl, C₁-C₄-haloalkoxy, C₃-C₆-cycloalkyl, C₃-C₆-cycloalkyl-C₁-C₄-alkyl-, C₃-C₆-cycloalkyl-O—, 4- to 6-membered heterocycloalkyl or —NR⁹R¹⁰;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹ represents phenyl or monocyclic heteroaryl,
   optionally substituted one to two times, independently from each other, with halogen, hydroxy, C₁-C₄-alkyl, C₁-C₄-alkoxy, C₁-C₄-haloalkyl, C₁-C₄-haloalkoxy, C₃-C₆-cycloalkyl, C₃-C₆-cycloalkyl-C₁-C₄-alkyl-, C₃-C₆-cycloalkyl-O—, 5- to 6-membered heterocycloalkyl or —NR⁹R¹⁰;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹ represents phenyl, pyridinyl, pyridazinyl, pyrimidinyl, pyrazinyl, furanyl, thiophenyl, pyrolyl, 1,2-thiazolyl, oxazolyl, triazolyl, imidazolyl, oxazolyl, pyrazolyl, oxadiazolyl, or imidazpyridinyl, optionally substituted once or twice, independently from each other, with fluoro, chloro, bromo, cyano, C₁-C₄-alkyl, methoxy, trifluoromethyl, difluoromethoxy, trifluoromethoxy, cyclopropyl, cyclobutyl, cyclopropylmethyl, oxanyl or —N(CH₃)₂;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹ represents phenyl, pyridinyl, pyridazinyl, furanyl, oxazolyl, pyrazolyl or oxadiazolyl, optionally substituted once or twice, independently from each other, with fluoro, chloro, C₁-C₄-alkyl, methoxy, trifluoromethyl, trifluoromethoxy, cyclopropyl, oxanyl or —N(CH₃)₂;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R² represents hydrogen, C₁-C₄-alkyl, C₁-C₄-haloalkyl or C₃-C₆-cycloalkyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R² represents hydrogen or C₁-C₄-alkyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R³ represents hydrogen, C₁-C₆-alkyl, phenyl or phenyl-C₁-C₃-alkyl, wherein
   said C₁-C₆-alkyl group is optionally substituted, one or more times, independently from each other, with hydroxy, halogen, C₁-C₄-alkoxy, —S(O)ₙ—C₁-C₄-alkyl, phenyl-C₁-C₃-alkoxy or —NR⁹R¹⁰ and
   said phenyl groups are optionally substituted, one or more times, independently from each other, with hydroxy, halogen, cyano, C₁-C₃-alkyl, C₁-C₃-haloalkyl, C₁-C₃-alkoxy or C₁-C₃-haloalkoxy;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R³ represents hydrogen, C₁-C₄-alkyl, phenyl or phenyl-methyl, wherein
   said C₁-C₄-alkyl group is optionally substituted once with hydroxy, methoxy, —S(O)ₙ-methyl, phenyl-methoxy or —NR⁹R¹⁰ and
   said phenyl groups are optionally substituted once with hydroxy;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R² and R³ together with the carbon atom to which they are attached form a 3- to 6-membered ring, said ring optionally containing one heteroatom selected from O, S, NH, NR^{a} in which R^{a} represents a C₁-C₄-alkyl group;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R² and R³ together with the carbon atom to which they are attached form a 3- to 6-membered ring, said ring optionally containing one oxygen atom;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁴ represents hydroxy, C₁-C₄-alkoxy or —NR¹¹R¹²;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁴ represents hydroxy, methoxy or —NR¹¹R¹²;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R² and R⁴ together represent *—C₂-C₅-alkanediyl-X¹—**, *—C₁-C₂-alkanediyl-X²—C₁-C₃-alkanediyl-** or *—C₁-C₂-alkanediyl-X²—C₂-C₃-alkanediyl-X¹—** to form a 5- to 9-membered ring,
   wherein * indicates the point of attachment of said group for R² and ** indicates the point of attachment of said group for R⁴;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R² and R⁴ together represent a group selected from:

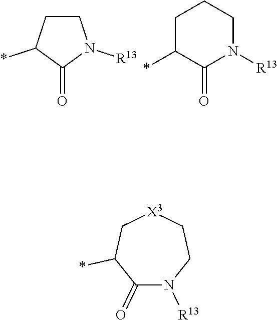

wherein * indicates the point of attachment of said group with the NH group in formula (I);
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁵ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 4- to 6-membered heterocycloalkyl, —CO₂—C₁-C₄-alkyl, —CO—NR⁹R¹⁰ or —NR⁹R¹⁰;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁵ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁵ represents hydrogen, halogen, C₁-C₄-alkyl, methoxy, trifluoromethyl or cyclopropyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁶ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁶ represents hydrogen, halogen or methyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁷ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁷ represents hydrogen, halogen, methyl or methoxy;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁸ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 1-R¹⁵—C₃-C₆-cycloalkyl, —CO₂—C₁-C₄-alkyl, —CO—NR⁹R¹⁰, —NR⁹R¹⁰, C₁-C₄-hydroxyalkyl, C₁-C₄-alkoxy-C₁-C₄-alkyl-, C₁-C₄-alkyl-S—, C₁-C₄-alkyl-S—C₁-C₄-alkyl-, —S(═O)R′, —S(═O)₂R′, —S(═O)₂NH₂, —S(═O)₂NHR′, —S(═O)₂N(R′)R″, —S(═O)(═NH)R′, 4- to 6-membered heterocycloalkyl, or —OR¹⁶;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁸ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 1-R¹⁵—C₃-C₆-cycloalkyl, —CO₂—C₁-C₄-alkyl, —CO—NR⁹R¹⁰ or —NR⁹R¹⁰;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁸ represents hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁸ represents hydrogen, halogen or methyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁵, R⁶, R⁷, R⁸ represent, independently from each other, hydrogen, fluoro, chloro, bromo, methyl, methoxy, trifluoromethyl or cyclopropyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁹ and R¹⁰ are the same or different and represent, independently from each other, hydrogen, C₁-C₃-alkyl or tert-butoxycarbonyl, or
   together with the nitrogen atom to which they are attached form a 4- to 6-membered nitrogen containing heterocyclic ring, said ring optionally containing one additional heteroatom selected from O, S, NH, NR^{a} in which R^{a} represents C₁-C₄-alkyl or C₁-C₄-alkoxycarbonyl;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁹ and R¹⁰ are the same or different and represent, independently from each other, hydrogen, C₁-C₃-alkyl or tert-butoxycarbonyl, or
   together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R⁹ and R¹⁰ are the same or different and represent, independently from each other, hydrogen, methyl or tert-butoxycarbonyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹¹ and R¹² are the same or different and represent, independently from each other, hydrogen, C₁-C₄-alkyl, C₂-C₄-hydroxyalkyl, C₁-C₄-alkoxy-C₂-C₄-alkyl-, R⁹R¹⁰N—C₂-C₄-alkyl-, C₃-C₆-cycloalkyl, 4- to 7-membered heterocycloalkyl, said 4- to 7-membered heterocycloalkyl group is optionally substituted, one or two times, independently from each other, with hydroxy, oxo, halogen, C₁-C₄-alkyl, C₁-C₄-alkoxy, or —NR⁹R¹⁰, or
   together with the nitrogen atom to which they are attached form a 4- to 6-membered nitrogen containing heterocyclic ring, said ring optionally containing one additional heteroatom selected from O, S, NH, NR^{a} in which R^{a} represents C₁-C₄-alkyl or C₁-C₄-alkoxycarbonyl and is optionally substituted, one or two times, independently from each other, with hydroxy, halogen, C₁-C₄-alkyl, C₁-C₄-alkoxy, or —NR⁹R¹⁰, or
   together with the nitrogen atom to which they are attached form a heterospirocycloalkyl group, which is optionally substituted, one or two times, independently from each other, with hydroxy, halogen, C₁-C₄-alkyl, C₁-C₄-alkoxy, or —NR⁹R¹⁰, or
   together with the nitrogen atom to which they are attached form a bridged heterocycloalkyl group, which is optionally substituted, one or two times, independently from each other, with hydroxy, halogen, C₁-C₄-alkyl, C₁-C₄-alkoxy, or —NR⁹R¹⁰;
      their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹¹ and R¹² are the same or different and represent, independently from each other, hydrogen, C₁-C₄-alkyl or C₃-C₆-cycloalkyl, wherein said C₁-C₄-alkyl group is optionally substituted with hydroxy;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹¹ and R¹² are the same or different and represent, independently from each other, hydrogen, C₁-C₃-alkyl or C₃-C₄-cycloalkyl, wherein said C₁-C₃-alkyl group is optionally substituted with hydroxy;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹³ represents hydrogen, C₁-C₄-alkyl, benzyl, 4-methoxybenzyl or tert-butoxycarbonyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹³ represents hydrogen or methyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹⁴ represents hydrogen, C₁-C₄-alkyl, benzyl or 4-methoxybenzyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹⁵ represents C₁-C₃-alkyl or C₁-C₃-haloalkyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹⁵ represents methyl or trifluoromethyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R¹⁶ represents C₂-C₆-hydroxyalkyl, C₁-C₄-alkoxy-C₂-C₆-alkyl-, or C₃-C₆-cycloalkyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
R′ and R″ represent, independently from each other, C₁-C₆-alkyl, C₁-C₆-haloalkyl, or C₃-C₆-cycloalkyl;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
X¹ represents O, S(O)ₘ, or NR¹³;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
X¹ represents O or NR¹³;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
X² represents O, S(O)ₘ, or NR¹⁴;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
X² represents O or NR¹⁴;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
X³ represents CH₂ or NR¹⁴;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
X³ represents CH₂ or NH;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
m represents 0, 1 or 2;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
m represents O or 2;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
m represents 2;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
n represents 0, 1 or 2;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
n represents O or 2;
   their polymorphs, enantiomeres, diastereomeres, racemates, tautomeres, N-oxides, hydrates and solvates, as well as their physiological acceptable salts and solvates of these salts, as well as mixtures of the same.

In a further embodiment of the first aspect, the present invention covers compounds of formula (I), supra, in which:
n represents 2;
   their polymorphs, enantiomeres, diastereomeres, racemates, tautomeres, N-oxides, hydrates and solvates, as well as their physiological acceptable salts and solvates of these salts, as well as mixtures of the same.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of intermediate compounds of general formula (V), supra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

The compounds according to the invention of general formula (I) can be prepared according to the following scheme 1. The scheme and procedures described below illustrate synthetic routes to the compounds of general formula (I) of the invention and are not intended to be limiting. It is clear to the person skilled in the art that the order of transformations as exemplified in scheme 1 can be modified in various ways. The order of transformations exemplified in this scheme is therefore not intended to be limiting. In addition, interconversion of any of the substituents R¹, R², R³, R⁴, R⁵, R⁶, R⁷ or R⁸ can be achieved before and/or after the exemplified transformations. These modifications can be such as the introduction of protecting groups, cleavage of protecting groups, reduction or oxidation of functional groups, halogenation, metallation, metal-catalysed coupling reactions, substitution or other reactions known to the person skilled in the art. These transformations include those which introduce a functionality which allows for further interconversion of substituents. Appropriate protecting groups and their introduction and cleavage are well-known to the person skilled in the art. Specific examples are described in the subsequent paragraphs.

Scheme 1 shows a route for the preparation of compounds of general formula (I).

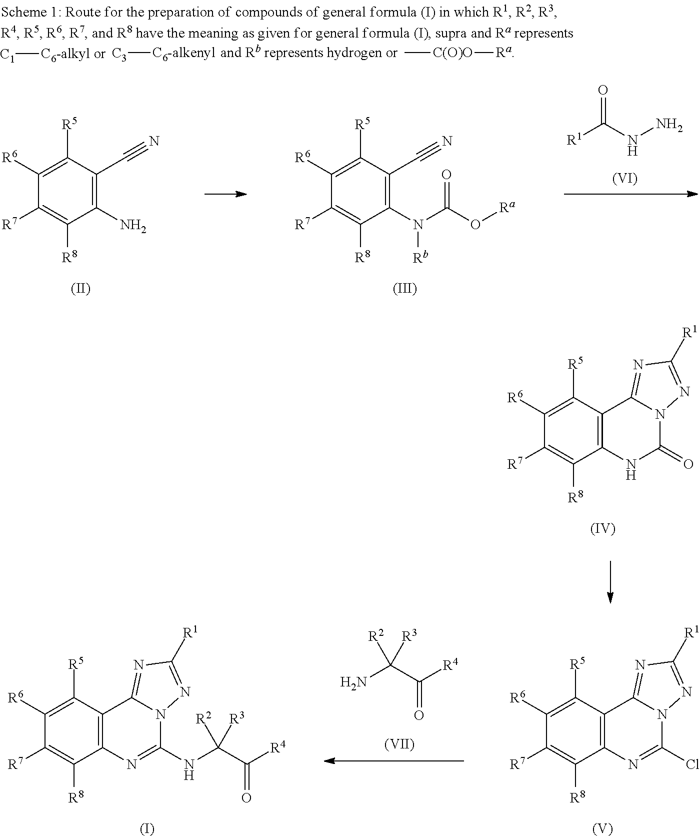

Scheme 2 describes another route for the preparation of compounds of formula (I).

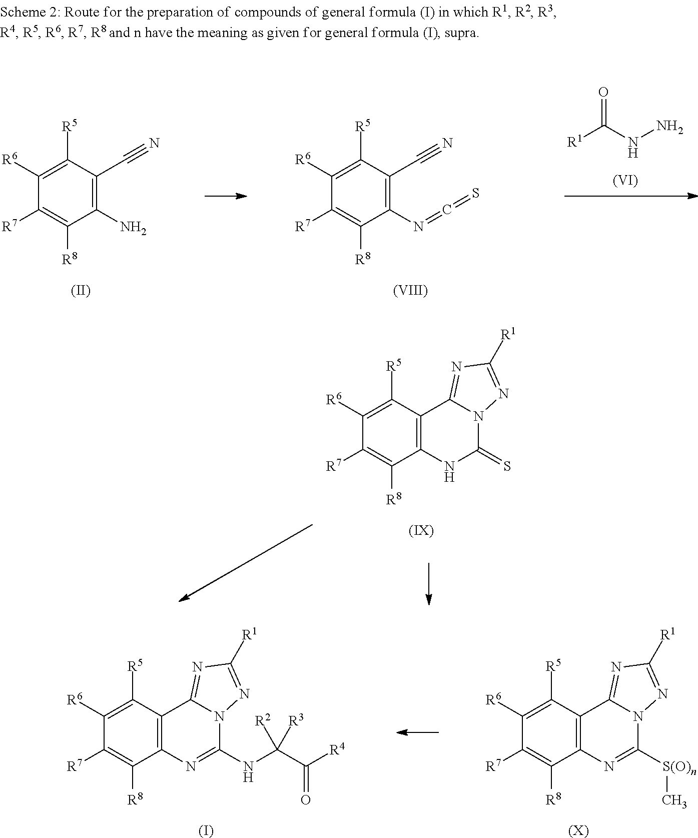

Scheme 3 describes an alternative route to prepare intermediates (IV).

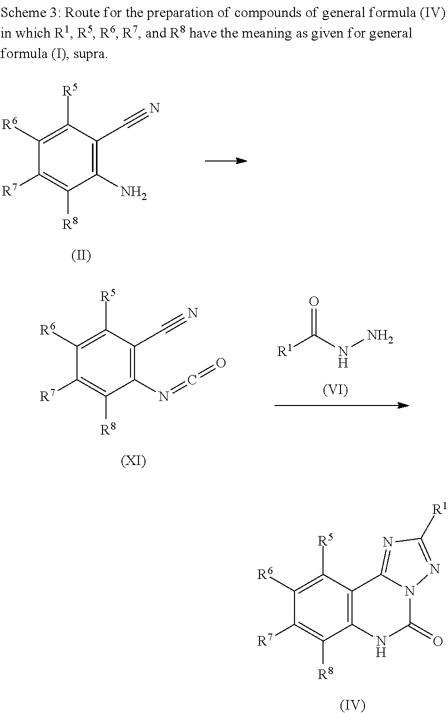

Scheme 4 describes another route for the preparation of compounds of formula (I).

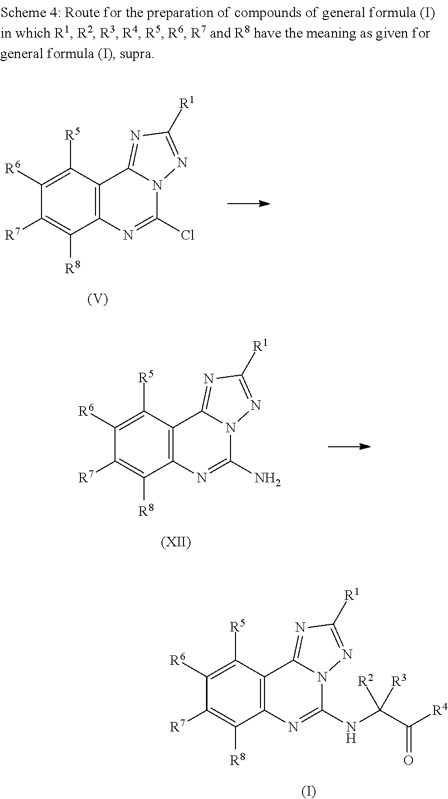

Scheme 5 describes another route for the preparation of compounds of formula (I).

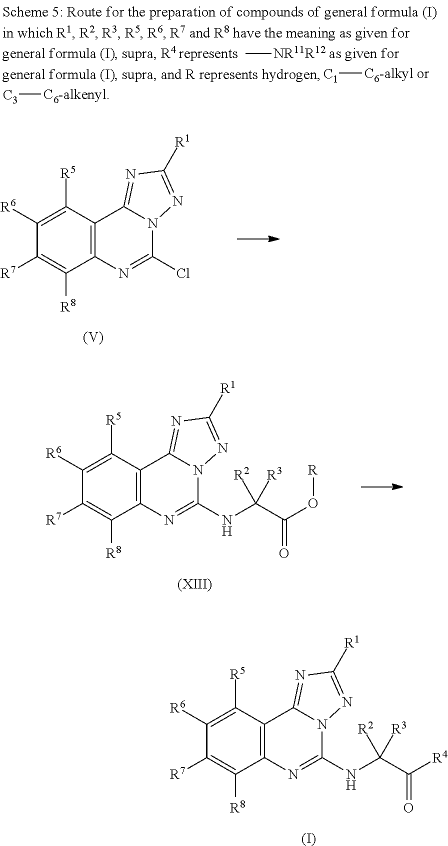

Scheme 6 describes an alternative route to prepare intermediates (IV) and (IX) respectively.

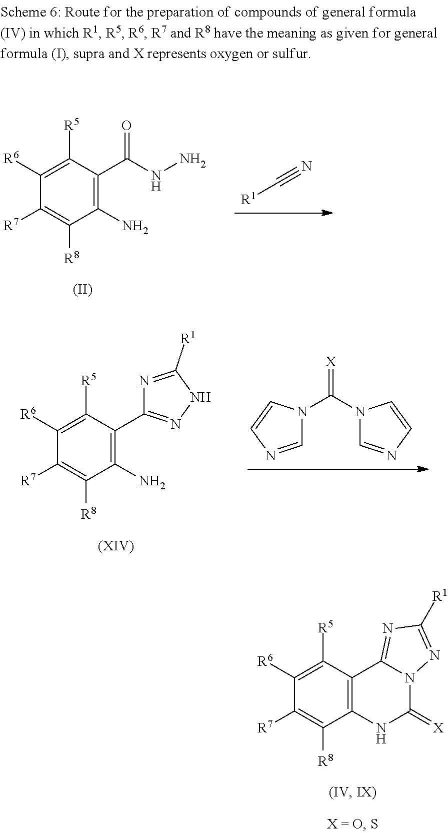

Scheme 7 describes an alternative route to prepare intermediates (IV) and (IX) respectively.

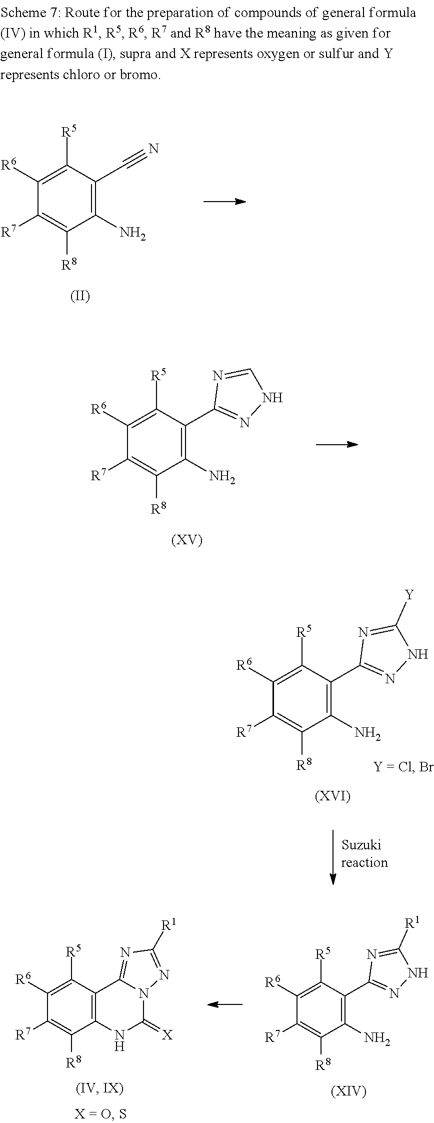

Scheme 8 describes an alternative route to prepare intermediates (IV).

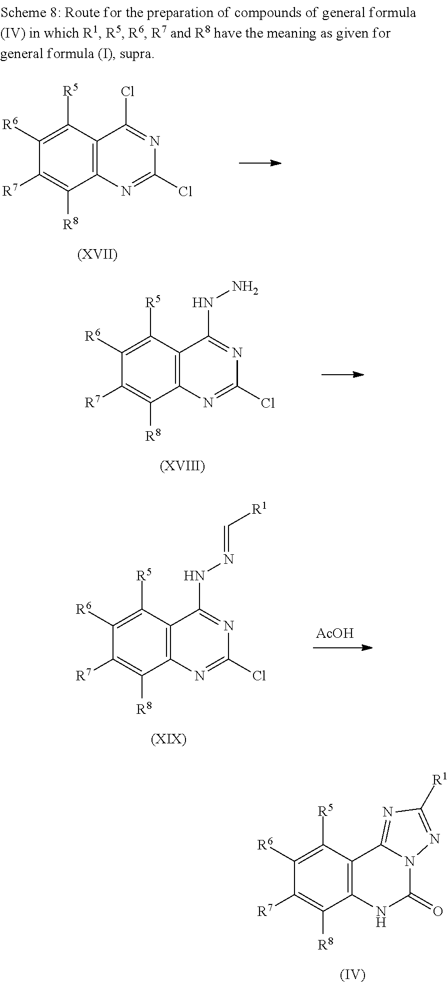

Scheme 9 describes another route for the preparation of compounds of formula (I).

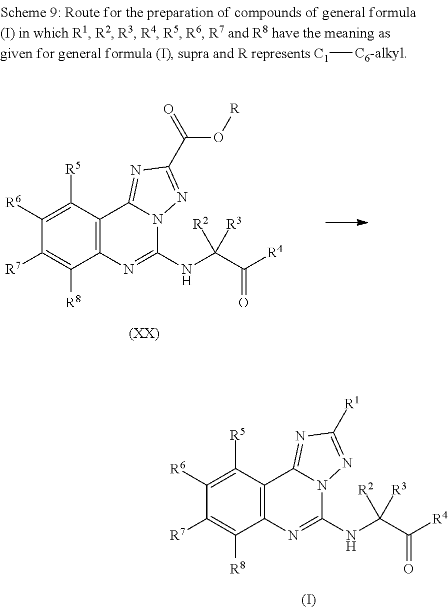

Compounds of general formula (I) of the present invention demonstrate a valuable pharmacological spectrum of action, which could not have been predicted. Compounds of the present invention have surprisingly been found to effectively inhibit AHR and it is possible therefore that said compounds be used for the treatment or prophylaxis of diseases, preferably cancer or conditions with dysregulated immune responses or other disorders associated with aberrant AHR signaling, in humans and animals.

Disorders and conditions particularly suitable for treatment with an AHR inhibitor of the present invention are liquid and solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, glioblastoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Examples of ovarian cancer include, but are not limited to serous tumour, endometrioid tumour, mucinous cystadenocarcinoma, granulosa cell tumour, Sertoli-Leydig cell tumour and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumours of the digestive tract include, but are not limited to, anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumours.

Tumours of the urinary tract include, but are not limited to, bladder, penile, kidney, renal pelvis, ureter, urethral and human papillary renal cancers.

Examples of kidney cancer include, but are not limited to renal cell carcinoma, urothelial cell carcinoma, juxtaglomerular cell tumour (reninoma), angiomyolipoma, renal oncocytoma, Bellini duct carcinoma, clear-cell sarcoma of the kidney, mesoblastic nephroma and Wilms' tumour. Examples of bladder cancer include, but are not limited to transitional cell carcinoma, squamous cell carcinoma, adenocarcinoma, sarcoma and small cell carcinoma.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to: yield better efficacy in reducing the growth of a tumour or even eliminate the tumour as compared to administration of either agent alone,

provide for the administration of lesser amounts of the administered chemotherapeutic agents,
provide for a chemotherapeutic treatment that is well tolerated in the patient with fewer deleterious pharmacological complications than observed with single agent chemotherapies and certain other combined therapies,
provide for treating a broader spectrum of different cancer types in mammals, especially humans,
provide for a higher response rate among treated patients,
provide for a longer survival time among treated patients compared to standard chemotherapy treatments,
provide a longer time for tumour progression, and/or
yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with: 131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, Enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the invention can further be combined with other reagents targeting the immune system, such as immune checkpoint inhibitors. Compositions comprising a PD-1/-L1 axis antagonist and an AHR antagonist and methods of using the same are provided herein. Data presented herein demonstrate that a combination of AHR inhibition and blockade of the PD-1/-L1 axis reduces the growth of tumor cells in more than an additive manner. PD-1, along with its ligands PD-L1 and PD-L2, function as negative regulators of T cell activation. AHR suppresses immune cell function while increasing cancer cell proliferation and motility. PD-L1 is overexpressed in many cancers and overexpression of PD-1 often occurs concomitantly in tumor infiltrating T cells. Thus results in attenuation of T cell activation and evasion of immune surveillance, which contributes to impaired antitumor immune responses. (Keir M E et al. (2008) Annu. Rev. Immunol. 26:677). Simultaneously targeting both the PD-1/-L1 axis and AHR enhances antitumor immune responses in more than an additive manner, leading to reduction of tumor growth that is unexpected. In some experiments, the resulting effect is greater than the expected or calculated additive effect of the individual components given separately. Thus, compositions comprising a PD-1/-L1 axis antagonist and an AHR antagonist are surprisingly effective in enhancing an immune response and in the treatment of cancer.

In addition, the inventive compounds can also be used as a therapeutic in a variety of other disorders wherein AHR is involved such as, cardiovascular and lung diseases.

Accordingly, the compounds according to the invention are suitable for the treatment and/or prophylaxis in particular of cardiovascular, inflammatory and fibrotic disorders and of renal disorders, in particular of acute and chronic renal insufficiency, and also of acute and chronic renal failure.

Accordingly, the compounds according to the invention can be used in medicaments for the treatment and/or prophylaxis of cardiovascular, inflammatory and fibrotic disorders, renal disorders, in particular of acute and chronic renal insufficiency, and also of acute and chronic renal failure.

For the purpose of the present invention the term renal insufficiency comprises both acute and chronic manifestations of renal insufficiency, and also underlying or related renal disorders such as diabetic and non-diabetic nephropathies, hypertensive nephropathies, ischaemic renal disorders, renal hypoperfusion, intradialytic hypotension, obstructive uropathy, renal stenoses, glomerulopathies, glomerulonephritis (such as, for example, primary glomerulonephritides; minimal change glomerulonephritis (lipoidnephrosis); membranous glomerulonephritis; focal segmental glomerulosclerosis (FSGS); membrane-proliferative glomerulonephritis; crescentic glomerulonephritis; mesangioproliferative glomerulonephritis (IgA nephritis, Berger's disease); post-infectious glomerulonephritis; secondary glomerulonephritides: diabetes mellitus, lupus erythematosus, amyloidosis, Goodpasture syndrome, Wegener granulomatosis, Henoch-Schönlein purpura, microscopic polyangiitis, acute glomerulonephritis, pyelonephritis (for example as a result of: urolithiasis, benign prostate hyperplasia, diabetes, malformations, abuse of analgesics, Crohn's disease), glomerulosclerosis, arteriolonecrose of the kidney, tubulointerstitial diseases, nephropathic disorders such as primary and congenital or aquired renal disorder, Alport syndrome, nephritis, immunological kidney disorders such as kidney transplant rejection and immunocomplex-induced renal disorders, nephropathy induced by toxic substances, nephropathy induced by contrast agents, diabetic and non-diabetic nephropathy, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or the need for dialysis. The present invention also comprises the use of the compounds according to the invention for the treatment and/or prophylaxis of sequelae of renal insufficiency, for example pulmonary oedema, heart failure, uremia, anemia, electrolyte disturbances (for example hypercalemia, hyponatremia) and disturbances in bone and carbohydrate metabolism.

The present invention also comprises the use of the compounds according to the invention for the treatment and/or prevention of sequelae of renal insufficiency, for example pulmonary oedema, heart failure, uraemia, anaemia, electrolyte disturbances (for example hyperkalaemia, hyponatraemia) and disturbances in bone and carbohydrate metabolism.

The compounds according to the invention are further suitable for the treatment and/or prevention of polycystic kidney disease (PCKD) and of the syndrome of inappropriate ADH secretion (SIADH).

Furthermore, the compounds according to the invention are also suitable for the treatment and/or prophylaxis of metabolic syndrome, hypertension, resistant hypertension, acute and chronic heart failure, coronary heart disease, stable and unstable angina pectoris, peripheral and cardiac vascular disorders, arrhythmias, atrial and ventricular arrhythmias and impaired conduction, for example atrioventricular blocks degrees I-III (AB block I-III), supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation, for example pulmonary oedema, cerebral oedema, renal oedema or oedema caused by heart failure, peripheral circulatory disturbances, reperfusion damage, arterial and venous thromboses, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for treatment and/or prophylaxis of erectile dysfunction and female sexual dysfunction.

In addition, the compounds according to the invention are also suitable for treatment and/or prophylaxis of asthmatic disorders, pulmonary arterial hypertension (PAH) and other forms of pulmonary hypertension (PH) including left-heart disease, HIV, sickle cell anaemia, thromboembolisms (CTEPH), sarcoidosis, COPD or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive dementia with corticobasal degeneration, amyolateral sclerosis (ALS), Huntington's disease, demyelinization, multiple sclerosis, thalamic degeneration, Creutzfeld-Jacob dementia, HIV dementia, schizophrenia with dementia or Korsakoffs psychosis. They are also suitable for treatment and/or prophylaxis of central nervous system disorders such as states of anxiety, tension and depression, CNS-related sexual dysfunctions and sleep disturbances, and for controlling pathological disturbances of the intake of food, stimulants and addictive substances. The compounds according to the invention are furthermore also suitable for controlling cerebral blood flow and thus represent effective agents for controlling migraines. They are also suitable for the prophylaxis and control of sequelae of cerebral infarction (cerebral apoplexy) such as stroke, cerebral ischaemia and craniocerebral trauma. The compounds according to the invention can likewise be used for controlling states of pain and tinnitus.

The compounds according to the invention are also suitable for treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds according to the invention are also suitable for controlling postoperative scarring, for example as a result of glaucoma operations.

The compounds according to the invention can also be used cosmetically for ageing and keratinized skin.

Moreover, the compounds according to the invention are suitable for treatment and/or prophylaxis of hepatitis, neoplasms, osteoporosis, glaucoma and gastroparesis.

The present invention further provides for the use of the compounds according to the invention for treatment and/or prophylaxis of disorders, especially the disorders mentioned above.

The present invention further provides for the use of the compounds according to the invention for the treatment and/or prophylaxis of chronic renal disorders, acute and chronic renal insufficiency, diabetic, inflammatory or hypertensive nephropaties, fibrotic disorders, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer and Creutzfeld-Jakob.

The present invention further provides a method for treatment and/or prophylaxis of disorders, in particular the disorders mentioned above, using an effective amount of at least one of the compounds according to the invention.

The present invention further provides a method for the treatment and/or prophylaxis of chronic renal disorders, acute and chronic renal insufficiency, diabetic, inflammatory or hypertensive nephropathies, fibrotic disorders, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer and Creutzfeld-Jakob.

In another embodiment, the inventive compounds can also be used to treat or to prevent uterine fibroids (uterine leiomyoma or uterine myoma) in women.

Uterine fibroids are benign tumors of the myometrium, the smooth muscle layer of the uterus. Uterine fibroids grow slowly during a women's life, and their growth is dependent on the female sexual hormones estradiol and progesterone [Kawaguchi K et al. Immunohistochemical analysis of oestrogen receptors, progesterone receptors and Ki-67 in leiomyoma and myometrium during the menstrual cycle and pregnancy Virchows Arch A Pathol Anat Histopathol. 1991; 419(4):309-15.], therefore the highest prevalence of uterine fibroids with approx. 70% and >80% in white and afro-american women, respectively, is found from 35 years of age onwards to menopause, when they shrink due to reduced hormone levels [Baird D D et al. High cumulative incidence of uterine leiomyoma in black and white women: Ultrasound evidence Am J Obstet Gynecol. 2003 January; 188(1):100-7.]. Approx 30% and 45% of white and afro-american women, respectively, do show clinically relevant symptoms due to their fibroids, which are heavy menstrual bleeding and pain, which is related to the menstrual cycle [David M et al. Myoma-associated pain frequency and intensity: a retrospective evaluation of 1548 myoma patients. Eur J Obstet Gynecol Reprod Biol. 2016 April; 199:137-40]. Heavy menstrual bleeding in this respect is defined by a blood loss of more than 80 mL in a menstrual bleeding period [Fraser I S et al. The FIGO Recommendations on Terminologies and Definitions for Normal and Abnormal Uterine Bleeding, Semin Reprod Med 2011; 29(5): 383-390]. Submucosal position of the uterine fibroids, e.g. those located directly below the endometrium, seems to have an even more severe effect on uterine bleeding, which may result in anemia in affected women [Yang J H et al. Impact of submucous myoma on the severity of anemia. Fertil Steril. 2011 April; 95(5):1769-72]. Furthermore, uterine fibroids, due to their symptoms, do severely affect the quality of life of affected women [Downes E et al. The burden of uterine fibroids in five European countries. Eur J Obstet Gynecol Reprod Biol. 2010 September; 152(1):96-102].

So far, it is not understood how uterine fibroids do cause heavy menstrual bleeding. Disregulated genes in uterine fibroids, in comparison to normal myometrium, can give a hint to understand the underlying mechanisms. In published and internal studies, we found TDO2, Tryptophan 2,3-dioxygenase, being highly upregulated [Tsibris J C et al. Insights from gene arrays on the development and growth regulation of uterine leiomyomata. Fertil Steril. 2002 July; 78(1):114-21.]. TDO2 metabolizes the substrate L-Tryptophan to L-Kynurenine, which can be further metabolized to kynurenic acid. Both, L-Kynurenine and Kynurenic acid are physiological ligands and activators for the arylhydrocarbon receptor AHR [Opitz C A et al. An endogenous tumour-promoting ligand of the human aryl hydrocarbon receptor Nature. 2011 Oct. 5; 478(7368):197-203].

L-Kynurenine controls at least two physiological processes which are dysregulated in uterine fibroids. L-Kynurenine, synthesized by an upregulation of IDO (Indoleamine-2,3-dyoxygenase) or TDO2, and acting via the AHR receptor, suppresses the immune system and thus prevents immune cells from recognizing and clearing the tumor cells [Munn D H Blocking IDO activity to enhance anti-tumor immunity. Front Biosci (Elite Ed). 2012 Jan. 1; 4:734-45]. Furthermore, an upregulation of L-Kynurenine leads to a vasodilation of vessels, and thus can directly increase blood loss and bleeding [Wang Y et al. Kynurenine is an endothelium-derived relaxing factor produced during inflammation Nature Medicine 16, 279-285 (2010)].

In summary, the upregulation of L-Kynurenine through activation of its physiological receptor AHR seems to support uterine fibroid growth by local suppression of the immune system, and might cause heavy menstrual bleeding by vasodilation of endometrial vessels in proximity to the tumor.

Therefore, a systemic or local application of compounds from the present invention inhibiting activation of the AHR and thus blocking the effect of uterine fibroid derived L-Kynurenine presents a new and valid treatment option for uterine fibroids.

Compounds of the present invention can be utilized to inhibit, block, reduce or decrease AHR activation by exogenous and/or endogenous ligands for the reduction of tumour growth and the modulation of dysregulated immune responses e.g. to block immunosuppression and increase immune cell activation and infiltration in the context of cancer and cancer immunotherapy; This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

The present invention also provides methods of treating a variety of other disorders wherein AHR is involved such as, but not limited to, inflammation, vaccination for infection & cancer, viral infections, obesity and diet-induced obesity, adiposity, metabolic disorders, hepatic steatosis and uterine fibroids.

These disorders have been well characterized in humans, but also exist with a similar etiology in other mammals, and can be treated by administering pharmaceutical compositions of the present invention.

The term “treating” or “treatment” as used in the present text is used conventionally, e.g., the management or care of a subject for the purpose of combating, alleviating, reducing, relieving, improving the condition of a disease or disorder, such as liquid and solid tumours.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular cancer or conditions with dysregulated immune responses or other disorders associated with aberrant AHR signaling.

The pharmaceutical activity of the compounds according to the invention can be explained by their activity as AHR inhibitors.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular cancer or conditions with dysregulated immune responses or other disorders associated with aberrant AHR signaling, particularly liquid and solid tumours.

In accordance with a further aspect, the present invention covers the use of a compound of formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of diseases, in particular cancer or conditions with dysregulated immune responses or other disorders associated with aberrant AHR signaling, particularly liquid and solid tumours.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular cancer or conditions with dysregulated immune responses or other disorders associated with aberrant AHR signaling, particularly liquid and solid tumours.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular cancer or conditions with dysregulated immune responses or other disorders associated with aberrant AHR signaling, particularly liquid and solid tumours. In accordance with a further aspect, the present invention covers a method of treatment or prophylaxis of diseases, in particular cancer or conditions with dysregulated immune responses or other disorders associated with aberrant AHR signaling, particularly liquid and solid tumours, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,

fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat), solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic*),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose), synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide), flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of cancer or conditions with dysregulated immune responses or other disorders associated with aberrant AHR signalinggeneric name disorders, particularly liquid and solid tumours.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

Based upon standard laboratory techniques known to evaluate compounds useful for the treatment of cancer or conditions with dysregulated immune responses or other disorders associated with aberrant AHR signaling, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Experimental Section

NMR peak forms are stated as they appear in the spectra, possible higher order effects have not been considered. The multiplicities are stated according to the signal form which appears in the spectrum, NMR-spectroscopic effects of a higher order were not taken into consideration. Multiplicity of the NMR signals: s=singlet, d=doublet, t=triplet, q=quartet, qi, quin=quintet, b, br=broad signal, m=multiplet. NMR signals: shift in ppm. Combinations of multiplicity could be e.g. dd=doublet from doublet.

Chemical names were generated using the ACD/Name software from ACD/Labs. In some cases generally accepted names of commercially available reagents were used in place of ACD/Name generated names.

Table 1 lists the abbreviations used in this paragraph and in the Examples section as far as they are not explained within the text body. Other abbreviations have their meanings customary per se to the skilled person.

TABLE 1
Abbreviations

| | |
|---|---|
| ACN | acetonitrile |
| AcOH | acetic acid |
| BPR | Back Pressure Regulator |
| CDCl₃ | deuterochloroform |
| DAD | diode array detector |
| DCM | dichloromethane |
| DEA | diethylamine |
| DIPEA | N,N-diisopropylethylamine |
| DMA | N,N-dimethylacetamide |
| DME | 1,2-dimethoxyethane |
| DMF | N,N-dimethylformamide |
| DMSO-d₆ | deuterated dimethyl sulfoxide |
| DMSO | dimethyl sulfoxide |
| EtOAc | ethyl acetate |
| EtOH | ethanol |
| Eq | equivalent |
| ESI | electrospray ionisation |
| Expl. | example |
| HATU | (7-aza-1H-benzotriazol-1-yl)-1,1,3,3-tetramethyluronium |
| | hexafluorophosphate |
| HBTU | O-benzotriazole- N,N,N′,N′-tetramethyluronium |
| | hexafluorophosphate |
| HPLC | high-pressure liquid chromatography |
| KA | kynurenic acid |
| LCMS | liquid chromatography coupled with mass spectrometry |
| LPS | lipopolysaccharide |
| mL | milliliter |
| min. | minute(s) |
| M | molar |
| mCPBA | meta chloro perbenzoic acid |
| MeLi | methyl lithium |
| MS | mass spectrometry |
| MTBE | methyl tert-butyl ether |
| MTP | microtiter plate |
| n-BuLi | n-butyl lithium |
| NMP | N-methyl-2-pyrrolidone |
| p | pressure |
| PBMC | peripheral blood mononuclear cells |
| Pd₂(dba)₃ | tris(dibenzylideneacetone)dipalladium(0) |
| Pd/C | Palladium on activated charcoal (10% with 50% water) |
| PLC | pressure liquid chromatography |
| PyBOB | (benzotriazol-1-yl)oxytripyrrolidinophosphonium |
| | hexafluorophosphate |
| RP-HPLC | reverse-phase high-pressure liquid chromatography |
| Rt | retention time |
| rt, r.t. | room temperature |
| sat. | saturated |
| T3P | 2,4,6-tripropyl-1,3,5,2,4,6-trioxatriphosphorinane 2,4,6- |
| | trioxide |
| tBuBrettPhos | [(2-Di-tert-butylphosphino-3,6-dimethoxy-2′,4′,6′- |
| Pd G3 | triisopropyl-1,1′-biphenyl)-2-(2′-amino-1,1′- |
| | biphenyl)]palladium(II) methanesulfonate |
| tBuBrettPhos | 2-(Di-tert-butylphosphino)-2′,4′,6′-triisopropyl-3,6- |
| | dimethoxy-1,1′-biphenyl |
| TEA | triethylamine |
| THF | tetrahydrofuran |
| TFA | trifluoroacetic acid |
| TLC | thin layer chromatography |
| TNFa | tumour necrosis factor alpha |
| μM | micromolar |
| UPLC | Ultra high performance chromatography |
| Xantphos | 4,5-bis(diphenylphosphino)-9,9-dimethylxanthene |
| Xphos | 2-dicyclohexylphosphino-2′,4′,6′-triisopropylbiphenyl |
| XPhos Pd G1 | [2-(2-aminoethyl)phenyl](chloro)palladium- |
| | dicyclohexyl(2′,4′,6′-triisopropyl[biphenyl]-2- |
| | yl)phosphine (1:1) |
| XPhos Pd G4 | methanesulfonato(2-dicyclohexylphosphino-2′,4′,6′-tri- |
| | iso-propy-1,1′-biphenyl)(2′-methylamino-1,1′-biphenyl-2- |
| | yl)palladium(II) |

The various aspects of the invention described in this application are illustrated by the following examples which are not meant to limit the invention in any way.

The example testing experiments described herein serve to illustrate the present invention and the invention is not limited to the examples given.

### EXPERIMENTAL SECTION—GENERAL PART

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil© or KP-NH® in combination with a Biotage autopurifier system (SP4© or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### UPLC/MS-Methods

### Method 1:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm.

### Method 2:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

### Method 3:

Column: XBridge BEH C18 2.5 μm 2.1×50 mm; Run Time: 4.70 min; Solvents: A) 10 mM ammonium bicarbonate pH 10, B) MeCN; Gradient: 2-98% B in 4.00 min, hold at 98% B to 4.70 min

### Method 4:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.7 min 1-45% B, 1.7-1.72 min 45-99% B, 1.72-2.0 min 99% B; flow 0.8 ml/min; temperature: 60° C.; DAD scan: 210-400 nm.

### Method 5:

Instrument: Waters Acquity UPLCMS SingleQuad; Column: Acquity UPLC CSH C18 1.7 μm 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### EXPERIMENTAL SECTION—INTERMEDIATES

### Intermediate 1

### methyl (2-cyanophenyl)carbamate

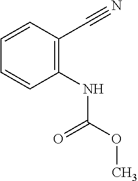

2-Aminobenzonitrile (CAS 1885-29-6, 3.84 g, 32.5 mmol) and potassium carbonate (13.5 g, 97.5 mmol) were solubilised in tetrahydrofuran (190 mL) and methyl carbonochloridate (CAS 79-22-1, 5.0 mL, 65 mmol) was added. The mixture was stirred at 80° C. overnight. The mixture was filtered, washed with tetrahydrofuran and concentrated under reduced pressure to give 6.01 g (80% purity, 84% yield) of the title compound.

LC-MS (Method 2): Rₜ=0.77 min; MS (ESIneg): m/z=175 [M−H]⁻.

¹H-NMR (400 MHz, DMSO-d6): δ [ppm]=3.69 (s, 3H), 7.33 (td, 1H), 7.52 (d, 1H), 7.63-7.71 (m, 1H), 7.79 (dd, 1H), 9.77 (s, 1H).

### Intermediate 2

### ethyl (2-cyano-6-fluorophenyl)carbamate

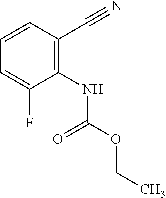

2-Amino-3-fluorobenzonitrile (500 mg, 3.67 mmol) was stirred in ethyl carbonochloridate (7.0 mL, 73 mmol) overnight at 100° C. The mixture was cooled to rt and concentrated under reduced pressure

LC-MS (method 2): Rₜ=0.81 min; MS (ESIneg): m/z=207 [M−H]⁻.

¹H-NMR (400 MHz, DMSO-d6): δ [ppm]=1.23 (t, 3H), 4.13 (q, 2H), 7.45-7.52 (m, 1H), 7.65-7.74 (m, 2H), 9.70 (br s, 1H).

### Intermediate 3

### methyl (2-cyano-6-methylphenyl)carbamate

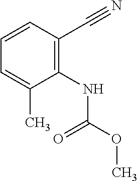

2-Amino-3-methylbenzonitrile (1.00 g, 7.57 mmol) and potassium carbonate (3.14 g, 22.7 mmol) were solubilised in toluene and methyl carbonochloridate (1.2 ml, 15 mmol) was added. The mixture was stirred at 80° C. overnight and 24 h at 120° C. The mixture was cooled to rt and filtered. The solid was washed with DCM and the filtrate was concentrated under reduced pressure to give 1.55 g (90% purity, 97% yield) of the title compound. The compound was used without further purification.

LC-MS (method 2): Rₜ=0.78 min; MS (ESIpos): m/z=191 [M+H]⁺

¹H-NMR (400 MHz, DMSO-d6): δ [ppm]=2.23 (s, 3H), 3.62-3.69 (m, 3H), 7.35 (t, 1H), 7.57-7.62 (m, 1H), 7.67 (d, 1H), 9.42- (s 1H).

### Intermediate 4

### methyl (2-cyano-5-methylphenyl)carbamate

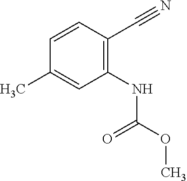

2-Amino-4-methylbenzonitrile (1.00 g, 7.57 mmol) and potassium carbonate (3.14 g, 22.7 mmol) were solubilised in THF (19 mL) and methyl carbonochloridate (1.2 mL, 15 mmol) was added. The mixture was stirred at 80° C. overnight. The reaction mixture was cooled to rt and filtered. The solid was washed with THF and the filtrate concentrated under reduced pressure to give 1.50 g (95% purity, 99% yield) of the title compound without further purification.

LC-MS (method 2): Rₜ=0.92 min; MS (ESIneg): m/z=189 [M−H]⁻.

¹H-NMR (400 MHz, DMSO-d6): δ [ppm]=2.36 (s, 3H), 3.68 (s, 3H), 7.13-7.17 (m, 1H), 7.33 (s, 1H), 7.67 (d, 1H), 9.69 (s, 1H).

### Intermediate 5

### methyl (2-cyano-5-fluorophenyl)carbamate

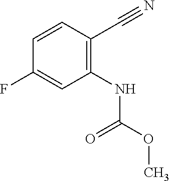

2-Amino-4-fluorobenzonitrile (1.00 g, 7.35 mmol) and potassium carbonate (3.05 g, 22.0 mmol) were solubilised in toluene (20 mL) and methyl carbonochloridate (1.1 mL, 15 mmol) was added. The mixture was stirred at 120° C. overnight. The mixture was cooled to rt and filtered. The solid was washed with toluene and the filtrate was concentrated under reduced pressure to give 1 g (72% yield) of the title compound. The compound was used without further purification.

LC-MS (Method 2): Rₜ=0.87 min; MS (ESIneg): m/z=193 [M−H]⁻.

¹H-NMR (400 MHz, DMSO-d6): δ [ppm]=3.71 (s, 3H), 7.21 (td, 1H), 7.47 (dd, 1H), 7.90 (dd, 1H), 9.98 (s, 1H).

### Intermediate 6

### methyl (2-cyano-4-methylphenyl)carbamate

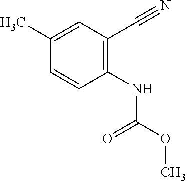

2-Amino-5-methylbenzonitrile (1.00 g, 7.57 mmol) and potassium carbonate (3.14 g, 22.7 mmol) were solubilised in THF (19 mL) and methyl carbonochloridate (1.2 mL, 15 mmol) was added. The mixture was stirred at 80° C. overnight. The reaction mixture was cooled to rt and filtered. The solid was washed with THF and the filtrate was concentrated under reduced pressure to give 1.55 g (95% purity, 102% yield) of the title compound.

LC-MS (method 2): Rₜ=0.93 min; MS (ESIpos): m/z=191 [M+H]⁺

¹H-NMR (400 MHz, DMSO-d6): δ [ppm]=2.31 (s, 3H), 3.67 (s, 3H), 7.37 (d, 1H), 7.46-7.50 (m, 1H), 7.61 (dd, 1H), 9.64 (s, 1H).

### Intermediate 7

### methyl (2-cyano-4-fluorophenyl)carbamate

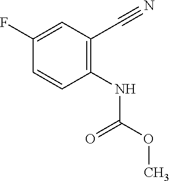

2-Amino-5-fluorobenzonitrile (1.00 g, 7.35 mmol) and potassium carbonate (3.05 g, 22.0 mmol) were solubilised in THF (20 mL) and methyl carbonochloridate (1.1 mL, 15 mmol) was added. The mixture was stirred at 80° C. overnight. The mixture was cooled to rt and filtered. The solid was washed with THF and the filtrate was concentrated under reduced pressure to give 1.6 g of the title compound. The compound was used without further purification.

LC-MS (Method 2): Rₜ=0.81 min; MS (ESIneg): m/z=193 [M−H]⁻.

¹H-NMR (400 MHz, DMSO-d6): δ [ppm]=3.68 (s, 3H), 7.49-7.62 (m, 2H), 7.82 (dd, 1H), 9.77 (s, 1H).

### Intermediate 8

### ethyl (3-bromo-2-cyanophenyl)carbamate

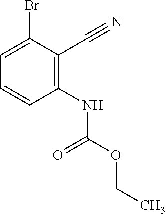

2-Amino-6-bromobenzonitrile (1.50 g, 7.61 mmol) was stirred in ethyl carbonochloridate (11 mL, 110 mmol) for 6 h at reflux. The reaction mixture was cooled to rt and concentrated under reduced pressure to give 2.21 g of the title compound. The compound was used without further purification.

LC-MS (method 2): Rₜ=1.05 min; MS (ESIneg): m/z=267 [M−H]⁻.

### Intermediate 9

### ethyl (3-chloro-2-cyanophenyl)carbamate

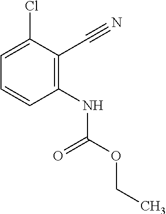

2-Amino-6-chlorobenzonitrile (1.75 g, 11.5 mmol) was stirred in ethyl carbonochloridate (20 mL, 210 mmol) for 6 h at reflux. The mixture was cooled to rt and concentrated under reduced pressure to give 3.00 g of the crude title compound. The compound was used without further purification.

LC-MS (method 2): Rₜ=1.06 min; MS (ESIneg): m/z=223 [M−H]⁻.

¹H-NMR (400 MHz, DMSO-d6): δ [ppm]=1.25 (t, 3H), 4.16 (q, 2H), 7.51 (d, 2H), 7.68 (dd, 1H), 9.95 (s, 1H).

### Intermediate 10

### ethyl [2-cyano-3-(trifluoromethyl)phenyl]carbamate

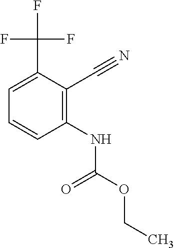 [SMILES: CCOC(=O)Nc1cccc(C(F)(F)F)c1C#N]

2-Amino-6-(trifluoromethyl)benzonitrile (500 mg, 2.69 mmol) was stirred in ethyl carbonochloridate (5.0 mL, 52 mmol) for 4 h at reflux. The reaction mixture was cooled to rt and concentrated under reduced pressure to give 740 mg of the crude title compound. The compound was used without further purification.

LC-MS (method 2): Rₜ=1.10 min; MS (ESIneg): m/z=257 [M−H]⁻.

### Intermediate 11

### 2-amino-6-cyclopropylbenzonitrile

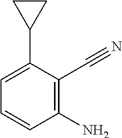 [SMILES: N#Cc1c(N)cccc1C1CC1]

2-Amino-6-bromobenzonitrile (500 mg, 2.54 mmol) was solubilised in 1,4-dioxane (25 mL). Cyclopropylboronic acid (262 mg, 3.05 mmol), cesium carbonate (71 μL, 10 mmol) and bis(diphenylphosphino)ferrocene-dichlorpalladium(II)-dichlormethane complex (414 mg, 508 μmol) were added and the reaction was stirred for 10 min at 130° C. under microwave irradiation. The reaction mixture was filtered and the solid was washed with dioxane. The filtrate was concentrated under reduced pressure to give 748 mg of the title compounds. The compound was used without further purification.

LC-MS (method 2): Rₜ=0.99 min; MS (ESIneg): m/z=157 [M−H]⁻.

¹H-NMR (400 MHz, DMSO-d6): δ [ppm]=0.65-0.71 (m, 2H), 0.96-1.03 (m, 2H), 1.94-2.05 (m, 1H), 5.90 (s, 2H), 6.15 (d, 1H), 6.56 (dd, 1H), 7.15 (t, 1H).

### Intermediate 12

### ethyl (2-cyano-3-cyclopropylphenyl)carbamate

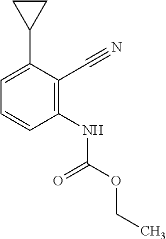 [SMILES: CCOC(=O)Nc1cccc(C2CC2)c1C#N]

2-Amino-6-cyclopropylbenzonitrile (700 mg, 4.42 mmol) was stirred in ethyl carbonochloridate (6.3 mL, 66 mmol) for 4 h at reflux. The reaction mixture was cooled to rt and concentrated under reduced pressure to give 901 mg (88% yield) of the title compound. The compound was used without further purification

LC-MS (method 2): Rₜ=1.11 min; MS (ESIpos): m/z=231 [M+H]⁺

### Intermediate 13

### methyl (2-cyano-3-methylphenyl)carbamate

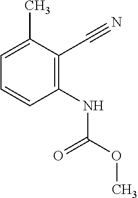

2-Amino-6-methylbenzonitrile (500 mg, 3.78 mmol) and potassium carbonate (1.57 g, 11.3 mmol) were solubilised in THF (9.6 mL) and methyl carbonochloridate (580 μL, 7.6 mmol) was carefully added. The mixture was stirred at 80° C. overnight. The reaction mixture was cooled to rt and filtered. The solid was washed with THF and the filtrate was concentrated under reduced pressure to give 788 mg of the title compound. The compound was used without further purification.

LC-MS (method 2): Rₜ=0.89 min; MS (ESIneg): m/z=189 [M−H]⁻

¹H-NMR (400 MHz, DMSO-d₆): δ [ppm]=2.46 (s, 3H), 3.68 (s, 3H), 7.24 (d, 1H), 7.33 (d, 1H), 7.50-7.58 (m, 1H), 9.69 (s, 1H).

### Intermediate 14

### methyl (2-cyano-3-fluorophenyl)carbamate

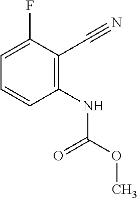

2-Amino-6-fluorobenzonitrile (150 mg, 1.10 mmol) and potassium carbonate (457 mg, 3.31 mmol) were stirred in toluene (2.8 mL) and methyl carbonochloridate (170 μl, 2.2 mmol) was added. The mixture was stirred at 120° C. overnight. The reaction mixture was cooled to rt and filtered. The filtrate was concentrated under reduced pressure to give 52 mg (100% purify, 24% yield) of the title compound. The compound was used without further purification.

LC-MS (Method 2): Rₜ=0.83 min; MS (ESIpos): m/z=193 [M+H]⁺

¹H-NMR (400 MHz, DMSO-d6): δ [ppm]=3.70 (s, 3H), 7.22-7.31 (m, 1H), 7.39 (d, 1H), 7.72 (td, 1H), 10.04 (s, 1H).

### Intermediate 15

### 2-phenyl[1,2,4]triazolo[1,5-c]quinazolin-5(6H)-one

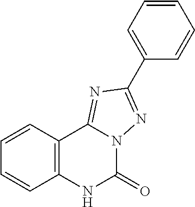 [SMILES: O=c1[nH]c2ccccc2c2nc(-c3ccccc3)nn12]

Methyl (2-cyanophenyl)carbamate (2.00 g, 11.4 mmol) and benzohydrazide (CAS 613-94-5, 1.85 g, 13.6 mmol) were stirred in N-methylpyrrolidone (50 mL) at 120° C. for 4 hours. Water was added to the mixture, precipitated product was filtered off, washed with water and dried under reduced pressure at 60° C. to give 2.09 g (90% purity, 63% yield) of the title compound.

LC-MS (Method 2): Rₜ=0.66 min; MS (ESIpos): m/z=263 [M+H]⁺

¹H-NMR (400 MHz, DMSO-d6): δ [ppm]=7.39-7.49 (m, 2H), 7.54-7.61 (m, 3H), 7.69-7.76 (m, 1H), 8.21-8.27 (m, 3H), 12.34 (s, 1H)

### Intermediate 16

### 5-chloro-2-phenyl[1,2,4]triazolo[1,5-c]quinazoline

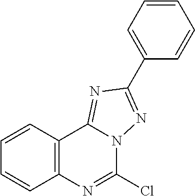 [SMILES: Clc1nc2ccccc2c2nc(-c3ccccc3)nn12]

2-Phenyl[1,2,4]triazolo[1,5-c]quinazolin-5(6H)-one (2.10 g, 8.02 mmol) was solubilised in phosphorus(V) oxychloride (40 mL, 430 mmol), N,N-diisopropylethylamine (2.8 mL, 16 mmol) was added carefully and the mixture was stirred overnight at 110° C. The mixture was concentrated and the residue was diluted with dichloromethane. Precipitated product was filtered off and washed with dichloromethane to give 1.83 g (100% purity, 81% yield) of the title compound.

C-MS (Method 2): Rₜ=1.39 min; MS (ESIpos): m/z=281 [M+H]⁺

¹H-NMR (500 MHz, DMSO-d₆): δ [ppm]=7.59-7.64 (m, 3H), 7.87 (td, 1H), 7.96-8.01 (m, 1H), 8.03-8.07 (m, 1H), 8.29-8.33 (m, 2H), 8.53 (dd, 1H)

### Intermediate 17

### 2-(4-chlorophenyl)[1,2,4]triazolo[1,5-c]quinazolin-5(6H)-one

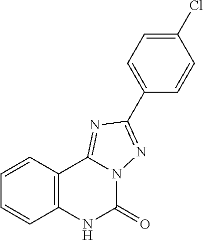 [SMILES: O=c1[nH]c2ccccc2c2nc(-c3ccc(Cl)cc3)nn12]

Methyl (2-cyanophenyl)carbamate (129 mg, 733 μmol) and 4-chlorobenzohydrazide (CAS 536-40-3, 150 mg, 879 μmol) were stirred in N-methylpyrrolidone (3.2 mL) at 120° C. for 4 hours. Water was added to the mixture, precipitated product was filtered off, washed with water and dried under reduced pressure at 60° C. to give 265 mg (90% purity, 110% yield) of the title compound.

LC-MS (Method 2): Rₜ=0.80 min; MS (ESIpos): m/z=297 [M+H]⁺

¹H-NMR (400 MHz, DMSO-d6): δ [ppm]=7.39-7.49 (m, 2H), 7.63-7.68 (m, 2H), 7.72 (ddd, 1H), 8.20-8.26 (m, 3H), 12.38 (br s, 1H)

### Intermediate 18

### 5-chloro-2-(4-chlorophenyl)[1,2,4]triazolo[1,5-c]quinazoline

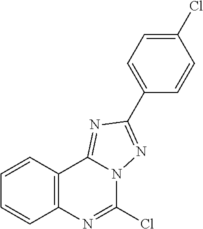 [SMILES: Clc1ccc(-c2nc3c4ccccc4nc(Cl)n3n2)cc1]

2-(4-Chlorophenyl)[1,2,4]triazolo[1,5-c]quinazolin-5(6H)-one (430 mg, 1.45 mmol) was solubilised in phosphorus(V) oxychloride (22 mL, 230 mmol), N,N-diisopropylethylamine (2.5 mL, 14 mmol) was added carefully and the mixture was stirred overnight at 110° C. The mixture was concentrated and the residue was diluted with dichloromethane. Precipitated product was filtered off and washed with dichloromethane to give 518 mg (113% yield) of the title compound.

LC-MS (Method 2): Rₜ=1.53 min; MS (ESIpos): m/z=315 [M+H]⁺

¹H-NMR (400 MHz, DMSO-d6) δ[ppm]: 1.207 (0.47), 1.218 (1.66), 1.234 (1.88), 1.252 (15.57), 1.269 (16.00), 1.273 (11.31), 1.288 (15.50), 1.305 (14.52), 3.072 (0.63), 3.083 (0.82), 3.091 (1.92), 3.101 (2.04), 3.109 (2.03), 3.119 (1.91), 3.138 (0.63), 3.492 (0.74), 3.519 (0.76), 3.549 (2.19), 3.563 (1.30), 3.577 (3.13), 3.589 (1.76), 3.596 (1.43), 3.606 (1.26), 3.622 (0.50), 5.944 (1.56), 7.416 (0.48), 7.481 (0.44), 7.501 (0.52), 7.631 (0.99), 7.652 (1.10), 7.665 (1.19), 7.686 (1.24), 7.718 (0.41), 7.870 (0.50), 7.983 (0.46), 8.034 (0.69), 8.209 (0.61), 8.215 (1.14), 8.236 (1.03), 8.287 (1.20), 8.308 (1.14), 8.504 (0.49), 8.523 (0.47), 9.428 (0.44), 12.433 (0.60).

### Intermediate 19

### 2-(3-fluorophenyl)[1,2,4]triazolo[1,5-c]quinazolin-5(6H)-one

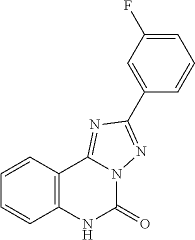 [SMILES: O=c1[nH]c2ccccc2c2nc(-c3cccc(F)c3)nn12]

Methyl (2-cyanophenyl)carbamate (150 mg, 851 μmol) and 3-fluorobenzohydrazide (CAS 499-55-8, 197 mg, 1.28 mmol) were stirred in N,N-dimethylformamide (3.0 mL) at 120° C. overnight. Water was added to the mixture, precipitated product was filtered off, washed with water and dried under reduced pressure at 60° C. to give 221 mg (98% purity, 91% yield) of the title compound.

LC-MS (Method 2): Rₜ=0.67 min; MS (ESIpos): m/z=281 [M+H]⁺

¹H-NMR (400 MHz, DMSO-d6): δ [ppm]=7.37-7.49 (m, 3H), 7.64 (td, 1H), 7.73 (ddd, 1H), 7.90-7.98 (m, 1H), 8.08 (dt, 1H), 8.24 (dd, 1H), 12.39 (br s, 1H)

### Intermediate 20

### 5-chloro-2-(3-fluorophenyl)[1,2,4]triazolo[1,5-c]quinazoline

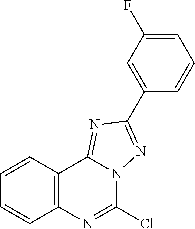 [SMILES: Fc1cccc(-c2nc3c4ccccc4nc(Cl)n3n2)c1]

2-(3-Fluorophenyl)[1,2,4]triazolo[1,5-c]quinazolin-5(6H)-one (1.35 g, 4.82 mmol) was solubilised in phosphorus(V) oxychloride (10 mL, 110 mmol), N,N-diisopropylethylamine (8.4 mL, 48 mmol) was added carefully and the mixture was stirred overnight at 110° C. The mixture was concentrated and the residue was diluted with dichloromethane. Precipitated product was filtered off and washed with dichloromethane to give 1.27 g (99% purity, 87% yield) of the title compound.

LC-MS (Method 2): Rₜ=1.42 min; MS (ESIpos): m/z=299 [M+H]⁺

¹H-NMR (400 MHz, DMSO-d6): δ [ppm]=7.43-7.49 (m, 1H), 7.67 (td, 1H), 7.88 (ddd, 1H), 7.96-8.02 (m, 2H), 8.03-8.07 (m, 1H), 8.15 (dt, 1H), 8.50-8.56 (m, 1H)

### Intermediate 21

### 2-(1-methyl-1H-pyrazol-4-yl)[1,2,4]triazolo[1,5-c]quinazolin-5(6H)-one

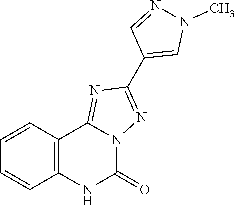 [SMILES: Cn1cc(-c2nc3c4ccccc4[nH]c(=O)n3n2)cn1]

Methyl (2-cyanophenyl)carbamate (1.04 g, 5.93 mmol) and 1-methyl-1H-pyrazole-4-carbohydrazide (CAS 170020-91-4, 831 mg, 5.93 mmol) were stirred in N,N-dimethylformamide (21 mL) at 120° C. for 20 hours. Water was added to the mixture, precipitated product was filtered off, washed with water and dried under reduced pressure at 60° C. to give 943 mg (95% purity, 57% yield) of the title compound.

LC-MS (Method 2): Rₜ=0.47 min; MS (ESIpos): m/z=267 [M+H]⁺

¹H-NMR (400 MHz, DMSO-d6): δ [ppm]=3.94 (s, 3H), 7.36-7.42 (m, 1H), 7.44 (d, 1H), 7.70 (ddd, 1H), 8.01 (d, 1H), 8.16 (dd, 1H), 8.42 (s, 1H), 12.26 (s 1H)

### Intermediate 22

### 5-chloro-2-(1-methyl-1H-pyrazol-4-yl)[1,2,4]triazolo[1,5-c]quinazoline

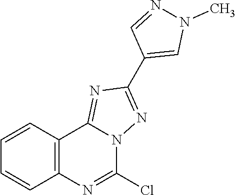 [SMILES: Cn1cc(-c2nc3c4ccccc4nc(Cl)n3n2)cn1]

2-(1-Methyl-1H-pyrazol-4-yl)[1,2,4]triazolo[1,5-c]quinazolin-5(6H)-one (943 mg, 3.54 mmol) was solubilised in phosphorus(V) oxychloride (10 mL, 107 mmol), N,N-diisopropylethylamine (6.2 mL, 35 mmol) was added carefully and the mixture was stirred overnight at 110° C. The mixture was concentrated and the residue was diluted with dichloromethane. Precipitated product was filtered off and washed with dichloromethane to give 963 mg (96% purity, 92% yield) of the title compound.

LC-MS (Method 2): Rₜ=0.97 min; MS (ESIpos): m/z=285 [M+H]⁺

¹H-NMR (400 MHz, DMSO-d6): δ [ppm]=3.96 (s, 3H), 7.84 (ddd, 1H), 7.96 (td, 1H), 7.99-8.04 (m, 1H), 8.09 (s, 1H), 8.45 (dd, 1H), 8.53 (s, 1H)

### Intermediate 23

### 2-(4-methoxyphenyl)[1,2,4]triazolo[1,5-c]quinazolin-5(6H)-one

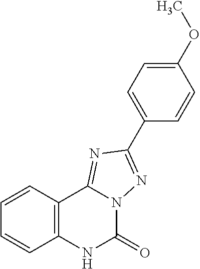 [SMILES: COc1ccc(-c2nc3c4ccccc4[nH]c(=O)n3n2)cc1]

Methyl (2-cyanophenyl)carbamate (2.00 g, 11.4 mmol) and 4-methoxybenzohydrazide (CAS 3290-99-1, 1.89 g, 11.4 mmol) were stirred in N,N-dimethylformamide (40 mL) at 120° C. for 20 hours. Water was added to the mixture, precipitated product was filtered off, washed with water and dried under reduced pressure at 60° C. to give 3.23 g (95% purity, 93% yield) of the title compound.

LC-MS (Method 2): Rₜ=0.69 min; MS (ESIpos): m/z=293 [M+H]⁺

¹H-NMR (400 MHz, DMSO-d6): δ [ppm]=3.85 (s, 3H), 7.10-7.15 (m, 2H), 7.37-7.47 (m, 2H), 7.71 (ddd, 1H), 8.14-8.19 (m, 2H), 8.22 (dd, 1H), (NH proton is not visible)

### Intermediate 24

### 5-chloro-2-(4-methoxyphenyl)[1,2,4]triazolo[1,5-c]quinazoline

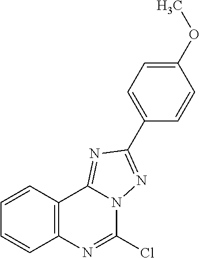 [SMILES: COc1ccc(-c2nc3c4ccccc4nc(Cl)n3n2)cc1]

2-(4-Methoxyphenyl)[1,2,4]triazolo[1,5-c]quinazolin-5(6H)-one (305 mg, 1.00 mmol) was solubilised in phosphorus(V) oxychloride (5 mL, 54 mmol), N,N-diisopropylethylamine (1.8 mL, 10 mmol) was added carefully and the mixture was stirred overnight at 110° C. The mixture was concentrated and the residue was diluted with dichloromethane. Precipitated product was filtered off and washed with dichloromethane to give 173 mg (100% purity, 53% yield) of the title compound.

LC-MS (Method 2): Rₜ=1.38 min; MS (ESIpos): m/z=311 [M+H]⁺

¹H-NMR (400 MHz, DMSO-d6): δ [ppm]=3.87 (s, 3H), 7.12-7.18 (m, 2H), 7.86 (td, 1H), 7.97 (td, 1H), 8.01-8.06 (m, 1H), 8.20-8.27 (m, 2H), 8.51 (dd, 1H)

### Intermediate 25

### 2-(4-methoxyphenyl)[1,2,4]triazolo[1,5-c]quinazoline-5(6H)-thione

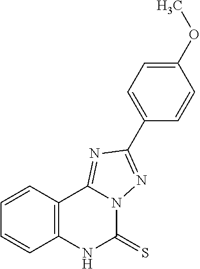 [SMILES: COc1ccc(-c2nc3c4ccccc4[nH]c(=S)n3n2)cc1]

2-Isothiocyanatobenzonitrile (CAS 81431-98-3, 100.0 mg, 624 μmol) and 4-methoxybenzohydrazide (CAS 3290-99-1, 103.4 mg, 624 μmol) were solubilised in isopropanol (20 mL, 261 mmol) and the mixture was stirred at reflux for 8 hours. The reaction was filtered and washed with isopropanol to give 177 mg (75% purity, 69% yield) of the title compound.

LC-MS (method 2): Rₜ=0.63 min; MS (ESIpos): m/z=309 [M+H]⁺

¹H-NMR (400 MHz, DMSO-d6): δ [ppm]=3.86 (s, 3H), 7.14 (d, 2H), 7.51-7.58 (m, 1H), 7.67 (d, 1H), 7.75-7.84 (m, 1H), 8.16-8.22 (m, 2H), 8.28 (dd, 1H), 13.96 (br s, 1H)

### Intermediate 26

### 2-(2-methylphenyl)[1,2,4]triazolo[1,5-c]quinazolin-5(6H)-one

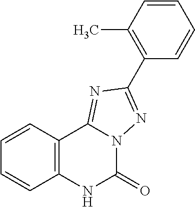 [SMILES: Cc1ccccc1-c1nc2c3ccccc3[nH]c(=O)n2n1]

Methyl (2-cyanophenyl)carbamate (500 mg, 2.84 mmol) and 2-methylbenzohydrazide (CAS 7658-80-2, 426 mg, 2.84 mmol) were stirred in N,N-dimethylformamide (130 mL) at 120° C. for 20 hours. Water was added to the mixture, precipitated product was filtered off, washed with water and dried under reduced pressure at 60° C. to give 666 mg (79% purity, 67% yield) of the title compound.

LC-MS (Method 2): Rₜ=0.72 min; MS (ESIpos): m/z=277 [M+H]⁺

¹H-NMR (400 MHz, DMSO-d6): δ [ppm]=2.70 (s, 3H), 7.35-7.48 (m, 5H), 7.72 (ddd, 1H), 8.09-8.14 (m, 1H), 8.23 (dd, 1H), 12.3 (s, 1H)

### Intermediate 27

### 5-chloro-2-(2-methylphenyl)[1,2,4]triazolo[1,5-c]quinazoline

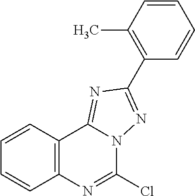 [SMILES: Cc1ccccc1-c1nc2c3ccccc3nc(Cl)n2n1]

2-(2-Methylphenyl)[1,2,4]triazolo[1,5-c]quinazolin-5(6H)-one (666 mg, 2.41 mmol) was solubilised in phosphorus(V) oxychloride (11 mL, 120 mmol), N,N-diisopropylethylamine (4.2 mL, 24 mmol) was added carefully and the mixture was stirred overnight at 110° C. The mixture was concentrated and the residue was diluted with dichloromethane. Precipitated product was filtered off and washed with dichloromethane to give 648 mg (100% purity, 91% yield) of the title compound.

LC-MS (Method 2): Rₜ=1.47 min; MS (ESIpos): m/z=295 [M+H]⁺

¹H-NMR (400 MHz, DMSO-d₆): δ [ppm]=2.75 (s, 3H), 7.36-7.51 (m, 3H), 7.87 (ddd, 1H), 7.94-8.00 (m, 1H), 8.03-8.07 (m, 1H), 8.13-8.21 (m, 1H), 8.52 (dd, 1H).

### Intermediate 28

### 2-[2-(trifluoromethyl)phenyl][1,2,4]triazolo[1,5-c]quinazolin-5(6H)-one

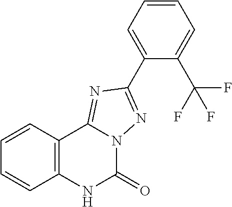 [SMILES: O=c1[nH]c2ccccc2c2nc(-c3ccccc3C(F)(F)F)nn12]

Methyl (2-cyanophenyl)carbamate (250 mg, 1.42 mmol) and 2-(trifluoromethyl)benzohydrazide (CAS 344-95-6, 348 mg, 1.70 mmol) were stirred in N,N-dimethylformamide (3 mL) at 120° C. for 15 hours. Water was added to the mixture, precipitated product was filtered off, washed with water and dried under reduced pressure at 60° C. to give 401 mg (86% yield) of the title compound.

LC-MS (Method 2): Rₜ=0.70 min; MS (ESIpos): m/z=331 [M+H]⁺

¹H-NMR (400 MHz, DMSO-d6) δ[ppm]: 1.229 (0.63), 2.331 (2.77), 2.518 (11.72), 2.523 (7.45), 2.727 (3.41), 2.888 (4.04), 2.934 (0.55), 3.072 (2.77), 3.365 (1.35), 3.383 (0.55), 3.868 (1.50), 6.834 (1.66), 6.852 (3.09), 6.870 (1.90), 6.872 (1.90), 6.925 (3.01), 6.944 (3.41), 7.022 (7.60), 7.042 (8.55), 7.049 (3.80), 7.070 (2.53), 7.097 (5.15), 7.099 (5.07), 7.117 (10.06), 7.135 (6.42), 7.138 (5.78), 7.181 (0.71), 7.261 (0.71), 7.284 (2.14), 7.287 (2.38), 7.305 (2.85), 7.322 (1.58), 7.325 (1.43), 7.400 (2.38), 7.402 (2.53), 7.421 (4.99), 7.429 (4.36), 7.438 (3.80), 7.440 (3.96), 7.450 (6.89), 7.465 (6.18), 7.485 (5.78), 7.511 (3.09), 7.566 (0.71), 7.648 (1.19), 7.667 (2.61), 7.685 (2.30), 7.699 (3.01), 7.716 (10.77), 7.733 (12.28), 7.736 (12.75), 7.748 (10.22), 7.774 (8.79), 7.792 (8.00), 7.800 (6.89), 7.820 (5.15), 7.840 (10.69), 7.859 (8.71), 7.885 (2.61), 7.904 (1.19), 7.953 (5.39), 7.965 (9.82), 7.985 (8.63), 8.006 (0.79), 8.101 (0.48), 8.175 (3.96), 8.178 (4.20), 8.195 (3.96), 8.198 (3.64), 10.172 (1.74), 10.571 (3.09), 10.662 (16.00), 10.909 (1.82), 11.254 (0.55).

### Intermediate 29

### 5-chloro-2-[2-(trifluoromethyl)phenyl][1,2,4]triazolo[1,5-c]quinazoline

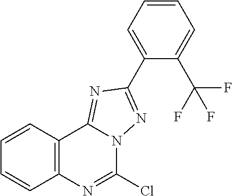 [SMILES: FC(F)(F)c1ccccc1-c1nc2c3ccccc3nc(Cl)n2n1]

2-[2-(Trifluoromethyl)phenyl][1,2,4]triazolo[1,5-c]quinazolin-5(6H)-one (401 mg, 1.21 mmol) was solubilised in phosphorus(V) oxychloride (4.0 mL, 43 mmol), N,N-diisopropylethylamine (2.1 mL, 12 mmol) was added carefully and the mixture was stirred overnight at 110° C. The mixture was poured into ice and stirred for one hour. Precipitated product was filtered off, washed with water and dried at 60° C. under reduced pressure to give 232 mg (55% yield) of the title compound.

LC-MS (Method 2): Rₜ=1.38 min; MS (ESIpos): m/z=349 [M+H]⁺

¹H-NMR (400 MHz, DMSO-d6): δ [ppm]=7.81-7.86 (m, 1H), 7.88-7.93 (m, 2H), 7.99-8.04 (m, 3H), 8.04-8.10 (m, 1H), 8.50 (dd, 1H).

### Intermediate 30

### 2-(3-methylphenyl)[1,2,4]triazolo[1,5-c]quinazolin-5(6H)-one

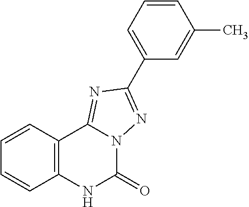 [SMILES: Cc1cccc(-c2nc3c4ccccc4[nH]c(=O)n3n2)c1]

Methyl (2-cyanophenyl)carbamate (1.50 g, 8.51 mmol) and 3-methylbenzohydrazide (CAS 13050-47-0, 1.28 g, 8.51 mmol) were stirred in N,N-dimethylformamide (30 mL) at 120° C. for 18 hours. Water was added to the mixture, precipitated product was filtered off, washed with water and dried under reduced pressure at 50° C. to give 1.87 g (79% yield, 100% purity) of the title compound.

LC-MS (method 3): Rₜ=0.73 min., MS (ESIpos): m/z=277 (M+H)⁺

¹H-NMR (400 MHz, methanol-d4): δ [ppm]=2.44 (s, 3H), 7.30-7.47 (m, 4H), 7.67-7.73 (m, 1H), 8.07 (d, 1H), 8.12 (s, 1H), 8.35 (d, 1H), NH not observed

### Intermediate 31

### 5-chloro-2-(3-methylphenyl)[1,2,4]triazolo[1,5-c]quinazoline

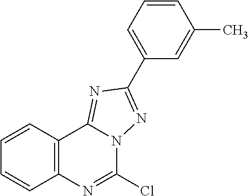 [SMILES: Cc1cccc(-c2nc3c4ccccc4nc(Cl)n3n2)c1]

2-(3-Methylphenyl)[1,2,4]triazolo[1,5-c]quinazolin-5(6H)-one (1.87 g, 6.77 mmol) was solubilised in phosphorus(V) oxychloride (21 mL, 230 mmol), N,N-diisopropylethylamine (12 mL, 68 mmol) was added carefully and the mixture was stirred 3 hours at 100° C. and left to stand at room temperature for additional 72 hours. The mixture was concentrated and the residue was diluted with water. Precipitated product was filtered off, washed with water and dried under vacuum at 50° C. to give a mixture of product and starting material. It was reused. Again it was dissolved in phosphorus(V) oxychloride (21 mL, 230 mmol) and N,N-diisopropylethylamine (12 mL, 68 mmol) was added carefully. The mixture was stirred 4 hours at 100° C. Solvent was evaporated and the residue was slowly added to ice. A solid precipitate was filtered off, washed with water and dried under reduced pressure at 50° C. to afford crude material as a brown solid. It was suspended in dichloromethane and the solid was filtered off affording starting material (120 mg). The filtrate was concentrated under reduced pressure to give 929.3 mg of the title compound (80% purity, 47% yield).

LC-MS (method 3): Rₜ=1.17 min., MS (ESIpos): m/z=295 (M+H)⁺

¹H-NMR (400 MHz, DMSO-d6): δ [ppm]=2.43 (s, 3H), 7.31-7.49 (m, 2H), 7.81-7.86 (m, 1H), 7.92-8.12 (m, 4H), 8.48-8.52 (m, 1H)

### Intermediate 32

### 2-[3-(trifluoromethyl)phenyl][1,2,4]triazolo[1,5-c]quinazolin-5(6H)-one

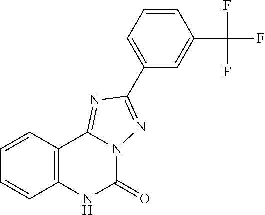 [SMILES: O=c1[nH]c2ccccc2c2nc(-c3cccc(C(F)(F)F)c3)nn12]

Methyl (2-cyanophenyl)carbamate (1.50 g, 8.51 mmol) and 3-(trifluoromethyl)benzohydrazide (CAS 22227-25-4, 1.74 g, 8.51 mmol) were stirred in N,N-dimethylformamide (30 mL) at 120° C. for 18 hours. Water was added to the mixture, precipitated product was filtered off, washed with water and dried under reduced pressure at 60° C. to give 1.72 g (61% yield, 94% purity) of the title compound.

LC-MS (method 3): Rₜ=0.79 min., MS (ESIpos): m/z=331 (M+H)⁺

¹H-NMR (400 MHz, DMSO-d6): δ [ppm]=7.39 (t, 1H), 7.43 (d, 1H), 7.66-7.72 (m, 1H), 7.81 (t, 1H), 7.89 (s, 1H), 8.23 (dd, 1H), 8.43 (s, 1H), 8.48 (d, 1H).

### Intermediate 33

### 5-chloro-2-[3-(trifluoromethyl)phenyl][1,2,4]triazolo[1,5-c]quinazoline

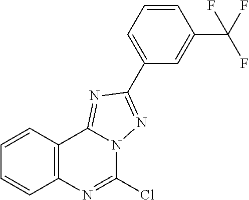 [SMILES: FC(F)(F)c1cccc(-c2nc3c4ccccc4nc(Cl)n3n2)c1]

2-[3-(Trifluoromethyl)phenyl][1,2,4]triazolo[1,5-c]quinazolin-5(6H)-one (1.70 g, 67% purity, 3.45 mmol) was added to phosphorus(V) oxychloride (20 mL, 210 mmol) at room temperature and N,N-diisopropylethylamine (6.0 mL, 34 mmol) was added slowly. The reaction mixture was warmed to 100° C. and stirred for 18 hours. Solvent was evaporated and the residue was poured into ice water and dichloromethane was added. The aqueous layer was extracted with dichloromethane thrice. The organic layers were combined, dried with magnesium sulfate and solvent was evaporated affording 1.88 g (119% yield, 76% purity) of the title compound.

LC-MS (method 3): Rₜ=1.19 min, (ESIpos): m/z=349 (M+H)⁺

¹H-NMR (400 MHz, DMSO-d6) δ[ppm]=7.82-7.87 (m, 2H), 7.93-8.04 (m, 3H), 8.47-8.57 (m, 3H).

### Intermediate 34

### 2-(2-fluorophenyl)[1,2,4]triazolo[1,5-c]quinazolin-5(6H)-one

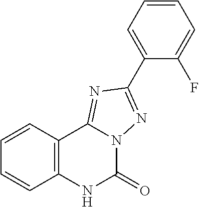 [SMILES: O=c1[nH]c2ccccc2c2nc(-c3ccccc3F)nn12]

Methyl (2-cyanophenyl)carbamate (1.50 g, 8.51 mmol) and 2-fluorobenzohydrazide (CAS 446-24-2, 1.31 g, 8.51 mmol) were stirred in N,N-dimethylformamide (30 mL) at 120° C. for 18 hours. Water was added to the mixture, precipitated product was filtered off, washed with water and dried under reduced pressure at 50° C. to give 1.66 g (69% yield, 99% purity) of the title compound.

LC-MS (method 3): Rₜ=0.59 min., MS (ESIpos): m/z=281 (M+H)⁺

¹H-NMR (400 MHz, DMSO-d6): δ [ppm]=7.36-7.46 (m, 4H), 7.55-7.61 (m, 1H), 7.67-7.72 (m, 1H), 8.16-8.22 (m, 2H), 12.32 (s, 1H)

### Intermediate 35

### 5-chloro-2-(2-fluorophenyl)[1,2,4]triazolo[1,5-c]quinazoline

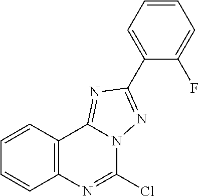 [SMILES: Fc1ccccc1-c1nc2c3ccccc3nc(Cl)n2n1]

2-(2-Fluorophenyl)[1,2,4]triazolo[1,5-c]quinazolin-5(6H)-one (1.66 g, 5.92 mmol) was solubilised in phosphorus(V) oxychloride (20 mL, 210 mmol), N,N-diisopropylethylamine (10 mL, 59 mmol) was added carefully and the mixture was stirred 3 hours at 100° C. and left to stand at room temperature for additional 72 hours. The mixture was concentrated and the residue was diluted with warm water. A precipitated solid was filtered off, washed with water and dried under vacuo at 50° C. to give a mixture of product and starting material as a brown solid. It was suspended in dichloromethane and the solid was filtered off. The filtrate was concentrated under reduced pressure to give 1.01 g of the title compound (90% purity, 51% yield).

LC-MS (method 3): Rₜ=1.04 min., MS (ESIpos): m/z=299 (M+H)⁺

¹H-NMR (400 MHz, DMSO-d6): δ [ppm]=7.36-7.47 (m, 2H), 7.59-7.66 (m, 1H), 7.82-7.87 (m, 1H), 7.93-7.99 (m, 1H), 8.03 (d, 1H), 8.27 (td, 1H), 8.49 (d, 1H), NH not observed

### Intermediate 36

### 2-(4-methylphenyl)[1,2,4]triazolo[1,5-c]quinazolin-5(6H)-one

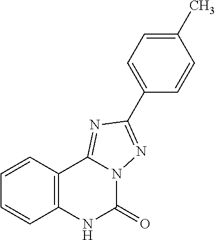 [SMILES: Cc1ccc(-c2nc3c4ccccc4[nH]c(=O)n3n2)cc1]

Methyl (2-cyanophenyl)carbamate (1.50 g, 8.51 mmol) and 4-methylbenzohydrazide (CAS 3619-22-5, 1.28 g, 8.51 mmol) were stirred in N,N-dimethylformamide (30 mL) at 120° C. for 18 hours. Water was added to the mixture, precipitated product was filtered off, washed with water and dried under reduced pressure at 50° C. to give 1.43 g (61% yield, 91% purity) of the title compound.

LC-MS (method 3): Rₜ=0.71 min., MS (ESIpos): m/z=277 (M+H)⁺

¹H-NMR (400 MHz, DMSO-d6): δ [ppm]=2.37 (s, 3H), 7.32-7.45 (m, 4H), 7.68 (t, 1H), 8.09 (d, 2H), 8.19 (d, 1H), 12.26 (br s, 1H)

### Intermediate 37

### 5-chloro-2-(4-methylphenyl)[1,2,4]triazolo[1,5-c]quinazoline

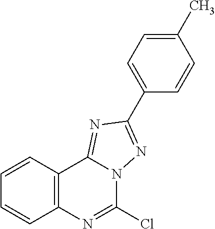 [SMILES: Cc1ccc(-c2nc3c4ccccc4nc(Cl)n3n2)cc1]

2-(4-Methylphenyl)[1,2,4]triazolo[1,5-c]quinazolin-5(6H)-one (340 mg, 1.23 mmol) was added to phosphorus(V) oxychloride (10 mL, 110 mmol) at room temperature and N,N-diisopropylethylamine (2.1 mL, 12 mmol) was added slowly. The reaction mixture was warmed to 100° C. and stirred for 72 hours. Solvent was evaporated and the residue was poured into ice water. The aqueous layer was extracted with dichloromethane thrice. The organic layers were combined, dried with magnesium sulfate and solvent was evaporated affording 471 mg (114% yield, 87% purity) of the title compound.

LC-MS (method 3): Rₜ=1.14 min., MS (ESIpos): m/z=295 (M+H)⁺

¹H-NMR (400 MHz, CDCl3): δ [ppm]=2.44 (s, 3H), 7.34 (d, 2H), 7.75 (t, 1H), 7.86 (t, 1H), 8.01 (d, 1H), 8.28 (d, 2H), 8.60 (d, 1H).

### Intermediate 38

### 2-[4-(trifluoromethyl)phenyl][1,2,4]triazolo[1,5-c]quinazolin-5(6H)-one

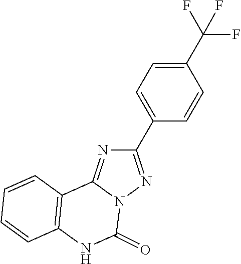 [SMILES: O=c1[nH]c2ccccc2c2nc(-c3ccc(C(F)(F)F)cc3)nn12]

Methyl (2-cyanophenyl)carbamate (250 mg, 1.42 mmol) and 4-(trifluoromethyl)benzohydrazide (CAS 339-59-3, 348 mg, 1.70 mmol) were stirred in N,N-dimethylformamide (3 mL) at 120° C. for 15 hours. Water was added to the mixture, precipitated product was filtered off, washed with water and dried under reduced pressure at 60° C. to give 491 mg (100% yield) of the title compound.

LC-MS (Method 2): Rₜ=0.83 min; MS (ESIpos): m/z=331 [M+H]⁺

¹H-NMR (400 MHz, DMSO-d6): δ [ppm]=7.40-7.49 (m, 2H), 7.73 (ddd, 1H), 7.96 (d, 2H), 8.25 (dd, 1H), 8.44 (d, 2H), 12.41 (s, 1H).

### Intermediate 39

### 5-chloro-2-[4-(trifluoromethyl)phenyl][1,2,4]triazolo[1,5-c]quinazoline

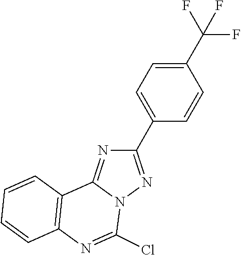 [SMILES: FC(F)(F)c1ccc(-c2nc3c4ccccc4nc(Cl)n3n2)cc1]

2-[4-(Trifluoromethyl)phenyl][1,2,4]triazolo[1,5-c]quinazolin-5(6H)-one (491 mg, 1.49 mmol) was solubilised in phosphorus(V) oxychloride (5.0 mL, 54 mmol), N,N-diisopropylethylamine (2.6 mL, 15 mmol) was added carefully and the mixture was stirred overnight at 110° C. The mixture was poured into ice and stirred for one hour. Precipitated product was filtered off, washed with water and dried at 60° C. under reduced pressure to give 475 mg (92% yield) of the title compound.

¹H-NMR (400 MHz, DMSO-d6): δ [ppm]=7.89 (ddd, 1H), 7.97-8.03 (m, 3H), 8.05-8.09 (m, 1H), 8.51 (d, 2H), 8.55 (dd, 1H).

### Intermediate 40

### 2-(2-chlorophenyl)[1,2,4]triazolo[1,5-c]quinazolin-5(6H)-one

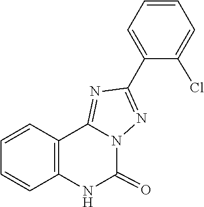 [SMILES: O=c1[nH]c2ccccc2c2nc(-c3ccccc3Cl)nn12]

Methyl (2-cyanophenyl)carbamate (1.75 g, 9.93 mmol) and 2-chlorobenzohydrazide (CAS 5814-05-1, 1.69 g, 9.93 mmol) were stirred in N,N-dimethylformamide (30 mL) at 120° C. for 96 hours and at 140° C. for further 18 hours. Water was added to the mixture, precipitated product was filtered off, washed with water and dried under reduced pressure at 50° C. to give 1.93 g (65% yield, 75% purity) of the title compound.

UPLC2-MS (short basic, 2-98%): Rₜ=0.61 min., MS (ESIpos): m/z=297 (M+H)⁺

¹H-NMR (400 MHz, MeOD-d3): δ [ppm]=7.40-7.55 (m, 4H), 7.58-7.62 (m, 1H), 7.69-7.75 (m, 1H), 7.91-7.95 (m, 1H), 8.30-8.34 (m, 1H).

### Intermediate 41

### 5-chloro-2-(2-chlorophenyl)[1,2,4]triazolo[1,5-c]quinazoline

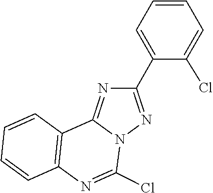 [SMILES: Clc1ccccc1-c1nc2c3ccccc3nc(Cl)n2n1]

2-(2-Chlorophenyl)[1,2,4]triazolo[1,5-c]quinazolin-5(6H)-one (170 mg, 573 μmol) was added to phosphorus(V) oxychloride (10 mL, 110 mmol) at room temperature and N,N-diisopropylethylamine (1000 μL, 5.7 mmol) was added slowly. The reaction mixture was warmed to 100° C. and stirred for 72 hours. The solvent was evaporated and azeotroped with toluene thrice to afford 2.69 g of the title compound as a dark brown oil. The material was taken onto next step without further purification.

UPLC2-MS (short basic, 2-98%): Rₜ=1.07 min., MS (ESIpos): m/z=317 (M+H)⁺.

### Intermediate 42

### 2-(3-chlorophenyl)[1,2,4]triazolo[1,5-c]quinazolin-5(6H)-one

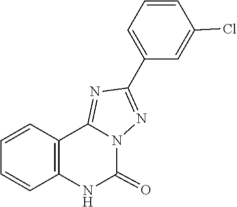 [SMILES: O=c1[nH]c2ccccc2c2nc(-c3cccc(Cl)c3)nn12]

Methyl (2-cyanophenyl)carbamate (1.60 g, 9.08 mmol) and 3-chlorobenzohydrazide (CAS 1673-47-8, 1.55 g, 9.08 mmol) were stirred in N,N-dimethylformamide (30 mL) at 120° C. for 72 hours. Water was added to the mixture, precipitated product was filtered off, washed with water and dried under reduced pressure at 50° C. to give 2.38 g (88% yield, 96% purity) of the title compound.

UPLC2-MS (short basic, 2-98%): Rₜ=0.74 min., MS (ESIpos): m/z=297 (M+H)⁺

¹H-NMR (400 MHz, DMSO-d6): δ [ppm]=7.39 (t, 1H), 7.43 (d, 1H), 7.56-7.62 (m, 2H), 7.67-7.72 (m, 1H), 8.13-8.18 (m, 2H), 8.21 (d, 1H), 12.34 (br s, 1H)

### Intermediate 43

### 5-chloro-2-(3-chlorophenyl)[1,2,4]triazolo[1,5-c]quinazoline

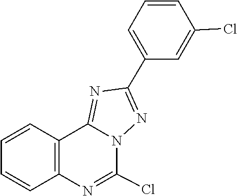 [SMILES: Clc1cccc(-c2nc3c4ccccc4nc(Cl)n3n2)c1]

2-(3-Chlorophenyl)[1,2,4]triazolo[1,5-c]quinazolin-5(6H)-one (700 mg, 2.36 mmol) was added to phosphorus(V) oxychloride (15 mL, 160 mmol) at room temperature and N,N-diisopropylethylamine (6.2 mL, 35 mmol) was added slowly. The reaction mixture was warmed to 100° C. and stirred for 24 hours. Solvent was evaporated and the residue was poured into ice water. The aqueous layer was extracted with dichloromethane thrice. The organic layers were combined, dried with magnesium sulfate and solvent was evaporated affording 1.31 g (79% yield, 45% purity) of the title compound.

UPLC2-MS (short basic, 2-98%): Rₜ=1.20 min., MS (ESIpos): m/z=315/317 (M+H)⁺ (product) and 0.73 min., 40.04%. MS (ESIpos): m/z=297/299 (M+H)⁺ (starting material)

### Intermediate 44

### 2-(2-methoxyphenyl)[1,2,4]triazolo[1,5-c]quinazolin-5(6H)-one

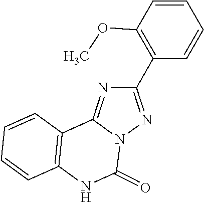 [SMILES: COc1ccccc1-c1nc2c3ccccc3[nH]c(=O)n2n1]

Methyl (2-cyanophenyl)carbamate (1.00 g, 5.68 mmol) and 2-methoxybenzohydrazide (CAS 7466-54-8, 1.13 g, 6.81 mmol) were stirred in N-methylpyrrolidone (25 mL) at 120° C. for 8 hours. Water was added to the mixture, precipitated product was filtered off, washed with water and dried under reduced pressure at 60° C. to give 846 mg (95% purity, 48% yield) of the title compound.

LC-MS (Method 2): Rₜ=0.65 min; MS (ESIpos): m/z=293 [M+H]⁺

¹H-NMR (400 MHz, DMSO-d6): δ [ppm]=3.87 (s, 3H), 7.12 (td, 1H), 7.22 (d, 1H), 7.38-7.43 (m, 1H), 7.45 (d, 1H), 7.49-7.56 (m, 1H), 7.71 (ddd, 1H), 7.92 (dd, 1H), 8.20 (dd, 1H), 12.31 (br s, 1H).

### Intermediate 45

### 5-chloro-2-(2-methoxyphenyl)[1,2,4]triazolo[1,5-c]quinazoline

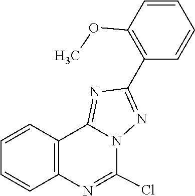 [SMILES: COc1ccccc1-c1nc2c3ccccc3nc(Cl)n2n1]

2-(2-Methoxyphenyl)[1,2,4]triazolo[1,5-c]quinazolin-5(6H)-one (846 mg, 2.89 mmol) was solubilised in phosphorus(V) oxychloride (14 mL, 150 mmol), N,N-diisopropylethylamine (5.0 mL, 29 mmol) was added carefully and the mixture was stirred overnight at 110° C. The mixture was poured into ice and stirred for one hour. Organic solvent was evaporated and precipitated product was filtered off, washed with water and dried at 60° C. under reduced pressure to give 455 mg (100% purity, 51% yield) of the title compound.

¹H-NMR (400 MHz, DMSO-d6): δ [ppm]=3.90 (s, 3H), 7.15 (td, 1H), 7.26 (d, 1H), 7.53-7.60 (m, 1H), 7.86 (ddd, 1H), 7.95-8.02 (m, 2H), 8.03-8.07 (m, 1H), 8.50 (dd, 1H).

LC-MS (Method 2): Rₜ=1.22 min; MS (ESIpos): m/z=311 [M+H]⁺

### Intermediate 46

### 2-(1H-pyrazol-3-yl)[1,2,4]triazolo[1,5-c]quinazolin-5(6H)-one

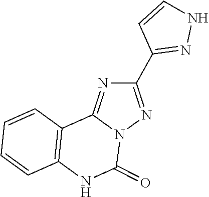 [SMILES: O=c1[nH]c2ccccc2c2nc(-c3cc[nH]n3)nn12]

Methyl (2-cyanophenyl)carbamate (250 mg, 1.42 mmol) and 1H-pyrazole-3-carbohydrazide (CAS 26275-64-9, 215 mg, 1.70 mmol) were stirred in N,N-dimethylformamide (3 mL) at 120° C. for 15 hours. Water was added to the mixture, precipitated product was filtered off, washed with water and dried under reduced pressure at 60° C. to give 290 mg (81% yield) of the title compound.

LC-MS (Method 2): Rₜ=0.46 min; MS (ESIpos): m/z=253 [M+H]⁺

¹H-NMR (400 MHz, DMSO-d6): δ [ppm]=6.86-6.97 (m, 1H), 7.38-7.50 (m, 2H), 7.71 (br t, 1H), 7.92 (s, 1H), 8.21 (d, 1H), 12.32 (br s, 1H), 13.31 (br s, 1H).

### Intermediate 47

### 5-chloro-2-(1H-pyrazol-3-yl)[1,2,4]triazolo[1,5-c]quinazoline

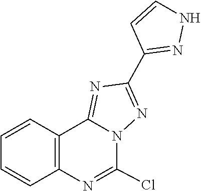 [SMILES: Clc1nc2ccccc2c2nc(-c3cc[nH]n3)nn12]

2-(1H-Pyrazol-3-yl)[1,2,4]triazolo[1,5-c]quinazolin-5(6H)-one (290 mg, 1.15 mmol) was solubilised in phosphorus(V) oxychloride (3.6 mL, 38 mmol), N,N-diisopropylethylamine (2.0 mL, 11 mmol) was added carefully and the mixture was stirred overnight at 110° C. The mixture was poured into ice and stirred for one hour. The organic solvent was evaporated and precipitated product was filtered off, washed with water and dried at 60° C. under reduced pressure to give 320 mg (38% purity, 39% yield) of the title compound.

LC-MS (Method 2): Rₜ=0.89 min; MS (ESIpos): m/z=271 [M+H]⁺ ¹H-NMR (400 MHz, DMSO-d6) δ [ppm]: 1.211 (0.61), 1.227 (0.90), 1.249 (6.94), 1.265 (8.14), 1.277 (6.62), 1.282 (2.63), 1.294 (6.39), 2.518 (4.33), 2.523 (2.80), 3.105 (0.86), 3.116 (0.84), 3.124 (0.84), 3.134 (0.84), 3.577 (0.48), 3.587 (0.50), 3.593 (0.67), 3.603 (0.65), 3.609 (0.46), 3.620 (0.48), 5.655 (0.82), 5.923 (1.07), 6.897 (13.77), 6.903 (16.00), 6.977 (5.84), 6.982 (6.16), 7.398 (2.80), 7.400 (3.03), 7.418 (5.49), 7.436 (3.15), 7.438 (3.41), 7.464 (5.11), 7.483 (6.14), 7.528 (0.50), 7.694 (3.55), 7.698 (3.68), 7.712 (3.53), 7.716 (4.58), 7.719 (3.28), 7.733 (2.52), 7.736 (2.48), 7.845 (2.02), 7.849 (12.36), 7.854 (11.58), 7.863 (2.14), 7.866 (2.35), 7.868 (1.58), 7.882 (1.58), 7.886 (1.58), 7.911 (5.28), 7.916 (5.28), 7.955 (1.26), 7.959 (1.32), 7.973 (0.97), 7.976 (2.31), 7.980 (2.12), 7.994 (1.70), 7.998 (1.62), 8.033 (2.69), 8.035 (2.88), 8.054 (1.58), 8.196 (4.39), 8.199 (4.60), 8.215 (4.44), 8.218 (4.08), 8.490 (1.96), 8.493 (2.02), 8.508 (1.79), 8.512 (1.83), 12.344 (6.43).

### Intermediate 48

### 2-(1-methyl-1H-pyrazol-3-yl)[1,2,4]triazolo[1,5-c]quinazolin-5(6H)-one

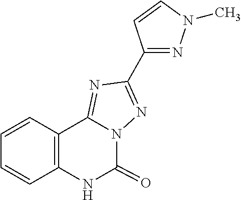 [SMILES: Cn1ccc(-c2nc3c4ccccc4[nH]c(=O)n3n2)n1]

Methyl (2-cyanophenyl)carbamate (503 mg, 2.86 mmol) and 1-methyl-1H-pyrazole-3-carbohydrazide (400 mg, 2.86 mmol) were stirred in DMF (10 mL) at 120° C. for 20 h. Water was added to the mixture and the solid was filtered, washed with water and dried under reduced pressure at 60° C. to give 630 mg of the title compound that was used without further purification.

LC-MS (method 2): Rₜ=0.48 min; MS (ESIpos): m/z=267 [M+H]⁺

### Intermediate 49

### 5-chloro-2-(1-methyl-1H-pyrazol-3-yl)[1,2,4]triazolo[1,5-c]quinazoline

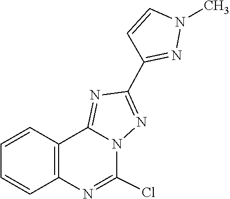 [SMILES: Cn1ccc(-c2nc3c4ccccc4nc(Cl)n3n2)n1]

2-(1-Methyl-1H-pyrazol-3-yl)[1,2,4]triazolo[1,5-c]quinazolin-5(6H)-one (630 mg, 2.37 mmol) was stirred in phosphorus(V) oxychloride (6.0 mL), N,N-diisopropylethylamine (4.1 mL, 24 mmol) was added carefully and the mixture was stirred for 3 h at 110° C. The mixture was poured into ice stirred for 1 h. The solid was filtered, washed with water and dried at 60° C. under reduced pressure to give 485 mg of the title compound that was used without further purification.

LC-MS (method 2): Rₜ=1.00 min; MS (ESIpos): m/z=285 [M+H]⁺

¹H-NMR (400 MHz, DMSO-d6): δ [ppm]=3.99 (s, 3H), 6.94 (d, 1H), 7.86 (ddd, 1H), 7.91 (d, 1H), 7.94-8.00 (m, 1H), 8.01-8.06 (m, 1H), 8.50 (dd, 1H).

### Intermediate 50

### 2-(5-methyl-1H-pyrazol-3-yl)[1,2,4]triazolo[1,5-c]quinazolin-5(6H)-one

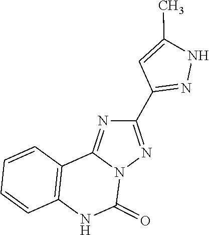 [SMILES: Cc1cc(-c2nc3c4ccccc4[nH]c(=O)n3n2)n[nH]1]

Methyl (2-cyanophenyl)carbamate (250 mg, 1.42 mmol) and 5-methyl-1H-pyrazole-3-carbohydrazide (CAS 40535-14-6, 199 mg, 1.42 mmol) were stirred in N,N-dimethylformamide (5 mL) at 120° C. for 40 hours. Water was added to the mixture, precipitated product was filtered off, washed with water and dried under reduced pressure at 60° C. to give 202 mg (70% purity, 37% yield) of the title compound. It was used without further purification.

LC-MS (Method 2): Rₜ=0.50 min; MS (ESIpos): m/z=267 [M+H]⁺

¹H-NMR (400 MHz, DMSO-d6) δ[ppm]: 2.287 (5.95), 2.321 (16.00), 2.522 (2.27), 2.669 (0.49), 2.726 (9.97), 2.885 (12.16), 6.469 (1.02), 6.624 (4.56), 6.698 (0.50), 7.000 (0.65), 7.019 (0.81), 7.064 (0.40), 7.081 (0.78), 7.101 (0.56), 7.390 (4.84), 7.408 (9.71), 7.428 (6.36), 7.437 (8.19), 7.457 (9.00), 7.684 (3.63), 7.703 (5.86), 7.722 (2.90), 7.948 (2.32), 7.966 (0.81), 8.177 (7.58), 8.180 (7.78), 8.197 (7.49), 10.547 (0.83), 10.616 (0.92), 12.290 (3.21), 12.970 (4.45), 13.552 (0.53).

### Intermediate 51

### 5-chloro-2-(5-methyl-1H-pyrazol-3-yl)[1,2,4]triazolo[1,5-c]quinazoline

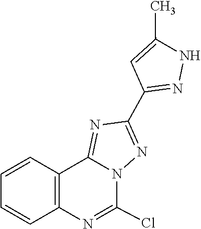 [SMILES: Cc1cc(-c2nc3c4ccccc4nc(Cl)n3n2)n[nH]1]

2-(5-Methyl-1H-pyrazol-3-yl)[1,2,4]triazolo[1,5-c]quinazolin-5(6H)-one (200 mg, 751 μmol) was solubilised in phosphorus(V) oxychloride (3.5 mL, 38 mmol), N,N-diisopropylethylamine (1.3 mL, 7.5 mmol) was added carefully and the mixture was stirred for two days at 110° C. The mixture was poured into ice and stirred for two hours. It was extracted with dichloromethane. The organic phase was dried over sodium sulfate and concentrated under reduced pressure to give 130 mg (92% purity, 61% yield) of the title compound. It was used without further purification.

LC-MS (Method 2): Rₜ=0.96 min; MS (ESIpos): m/z=285 [M+H]⁺

¹H-NMR (400 MHz, DMSO-d6) δ[ppm]: 1.196 (0.46), 1.205 (0.89), 1.213 (0.54), 1.220 (1.05), 1.237 (2.31), 1.246 (7.31), 1.255 (4.73), 1.262 (7.78), 1.269 (7.33), 1.286 (6.85), 2.310 (0.57), 2.331 (16.00), 2.518 (1.93), 2.523 (1.23), 2.664 (0.41), 2.669 (0.56), 3.114 (0.95), 3.124 (0.98), 3.133 (0.96), 3.143 (0.93), 3.568 (0.50), 3.585 (0.82), 3.594 (0.86), 3.601 (1.00), 3.611 (1.00), 3.618 (0.82), 3.627 (0.79), 3.643 (0.46), 5.759 (1.14), 6.723 (4.86), 6.725 (4.93), 7.838 (1.15), 7.841 (1.23), 7.856 (1.84), 7.858 (2.29), 7.861 (1.65), 7.875 (1.64), 7.878 (1.63), 7.948 (1.21), 7.952 (1.27), 7.969 (2.33), 7.973 (2.04), 7.987 (1.62), 7.990 (1.52), 8.025 (3.12), 8.044 (1.71), 8.085 (0.44), 8.472 (2.07), 8.474 (2.24), 8.492 (2.09), 8.494 (1.94).

### Intermediate 52

### 2-(1-methyl-1H-pyrazol-5-yl)[1,2,4]triazolo[1,5-c]quinazolin-5(6H)-one

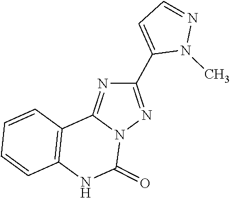 [SMILES: Cn1nccc1-c1nc2c3ccccc3[nH]c(=O)n2n1]

Methyl (2-cyanophenyl)carbamate (71.2 mg, 404 μmol) and 1-methyl-1H-pyrazole-5-carbohydrazide (85.0 mg, 607 μmol) were stirred in DMF (850 μL) overnight at 120° C. The reaction mixture was cooled to rt and diluted with water. The solid was filtered, washed with water and dried under reduced pressure at 60° C. to give 78.0 mg (72% yield) of the title compound without further purification.

LC-MS (Method 2): Rₜ=0.53 min; MS (ESIpos): m/z=267 [M+H]⁺

¹H-NMR (400 MHz, DMSO-d6): δ [ppm]=4.29 (s, 3H), 6.99 (d, 1H), 7.40-7.44 (m, 1H), 7.46 (d, 1H), 7.60 (d, 1H), 7.73 (ddd, 1H), 8.22 (dd, 1H), 12.43 (s, 1H).

### Intermediate 53

### 5-chloro-2-(1-methyl-1H-pyrazol-5-yl)[1,2,4]triazolo[1,5-c]quinazoline

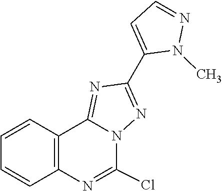 [SMILES: Cn1nccc1-c1nc2c3ccccc3nc(Cl)n2n1]

2-(1-Methyl-1H-pyrazol-5-yl)[1,2,4]triazolo[1,5-c]quinazolin-5(6H)-one (78.0 mg, 293 μmol) was solubilised in phosphorus(V) oxychloride (1.1 mL, 11 mmol), N,N-diisopropylethylamine (510 μL, 2.9 mmol) was added carefully and the mixture was stirred overnight at 110° C. The mixture was cooled to rt, was poured into ice and stirred for 1 h. The solid was filtered, washed with water and dried at 60° C. under reduced pressure to provide 50.0 mg (60% yield) the title compound without further purification.

LC-MS (Method 2): Rₜ=1.13 min; MS (ESIpos): m/z=286 [M+H]⁺

¹H-NMR (400 MHz, DMSO-d6): δ [ppm]=4.35 (s, 3H), 7.07 (d, 1H), 7.64 (d, 1H), 7.88 (ddd, 1H), 7.97-8.03 (m, 1H), 8.04-8.09 (m, 1H), 8.52 (dd, 1H).

### Intermediate 54

### 2-(1-ethyl-3-methyl-1H-pyrazol-4-yl)[1,2,4]triazolo[1,5-c]quinazolin-5(6H)-one

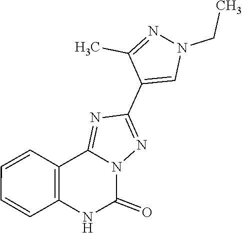 [SMILES: CCn1cc(-c2nc3c4ccccc4[nH]c(=O)n3n2)c(C)n1]

Methyl (2-cyanophenyl)carbamate (209 mg, 1.19 mmol) and 1-ethyl-3-methyl-1H-pyrazole-4-carbohydrazide (CAS 1177272-66-0, 200 mg, 1.19 mmol) were stirred in N,N-dimethylformamide (4.2 mL) at 120° C. for 20 hours. Water was added to the mixture, precipitated product was filtered off, washed with water and dried under reduced pressure at 60° C. to give 200 mg (98% purity, 56% yield) of the title compound.

LC-MS (Method 2): Rₜ=0.56 min; MS (ESIpos): m/z=295 [M+H]⁺

¹H-NMR (400 MHz, DMSO-d6): δ [ppm]=1.41 (t, 3H), 2.54 (s, 3H), 4.15 (q, 2H), 7.34-7.56 (m, 2H), 7.70 (ddd, 1H), 8.15 (dd, 1H), 8.35 (s, 1H), 12.26 (br s, 1H).

### Intermediate 55

### 5-chloro-2-(1-ethyl-3-methyl-1H-pyrazol-4-yl)[1,2,4]triazolo[1,5-c]quinazoline

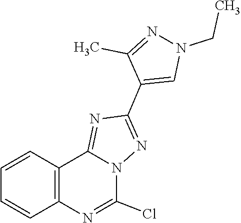 [SMILES: CCn1cc(-c2nc3c4ccccc4nc(Cl)n3n2)c(C)n1]

2-(1-Ethyl-3-methyl-1H-pyrazol-4-yl)[1,2,4]triazolo[1,5-c]quinazolin-5(6H)-one (200 mg, 678 μmol) was solubilised in phosphorus(V) oxychloride (1.9 mL, 20 mmol), N,N-diisopropylethylamine (1.2 mL, 6.8 mmol) was added carefully and the mixture was stirred overnight at 110° C. The mixture was poured into ice and stirred for two hours. Precipitated product was filtered off, washed with water and dried at 60° C. under reduced pressure to give 128 mg (66% purity, 40% yield) of the title compound. It was used without purification.

LC-MS (Method 2): Rₜ=1.18 min; MS (ESIpos): m/z=313 [M+H]⁺

¹H-NMR (400 MHz, DMSO-d6): δ [ppm]=1.42 (t, 3H), 2.53 (s, 3H), 4.16 (q, 2H), 7.84 (td, 1H), 7.95 (td, 1H), 8.02 (d, 1H), 8.44 (dd, 1H), 8.45 (s, 1H).

### Intermediate 56

### 2-(1-ethyl-1H-pyrazol-4-yl)[1,2,4]triazolo[1,5-c]quinazolin-5(6H)-one

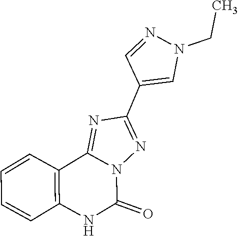 [SMILES: CCn1cc(-c2nc3c4ccccc4[nH]c(=O)n3n2)cn1]

Methyl (2-cyanophenyl)carbamate (229 mg, 1.30 mmol) and 1-ethyl-1H-pyrazole-4-carbohydrazide (CAS 512809-51-7, 200 mg, 1.30 mmol) were stirred in N,N-dimethylformamide (4.6 mL) at 120° C. for 20 hours. Water was added to the mixture, precipitated product was filtered off, washed with water and dried under reduced pressure at 60° C. to give 244 mg (100% purity, 67% yield) of the title compound.

LC-MS (Method 2): Rₜ=0.52 min; MS (ESIpos): m/z=281 [M+H]⁺

¹H-NMR (400 MHz, DMSO-d6): δ [ppm]=1.44 (t, 3H), 4.24 (q, 2H), 7.34-7.51 (m, 2H), 7.70 (ddd, 1H), 8.03 (d, 1H), 8.17 (dd, 1H), 8.46 (s, 1H), 12.26 (br s, 1H).

### Intermediate 57

### 5-chloro-2-(1-ethyl-1H-pyrazol-4-yl)[1,2,4]triazolo[1,5-c]quinazoline

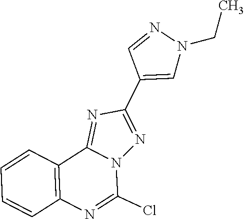 [SMILES: CCn1cc(-c2nc3c4ccccc4nc(Cl)n3n2)cn1]

2-(1-Ethyl-1H-pyrazol-4-yl)[1,2,4]triazolo[1,5-c]quinazolin-5(6H)-one (244 mg, 872 μmol) was solubilised in phosphorus(V) oxychloride (2.4 mL, 26 mmol), N,N-diisopropylethylamine (1.5 mL, 8.7 mmol) was added carefully and the mixture was stirred overnight at 110° C. The mixture was poured into ice and stirred for two hours. Precipitated product was filtered off, washed with water and dried at 60° C. under reduced pressure to give 133 mg (85% purity, 43% yield) of the title compound. It was used without purification.

LC-MS (Method 2): Rₜ=1.06 min; MS (ESIpos): m/z=299 [M+H]⁺

¹H-NMR (400 MHz, DMSO-d6): δ [ppm]=1.45 (t, 3H), 4.25 (q, 2H), 7.84 (td, 1H), 7.96 (td, 1H), 8.02 (dd, 1H), 8.10 (s, 1H), 8.45 (dd, 1H), 8.57 (s, 1H).

### Intermediate 58

### 2-(1,5-dimethyl-1H-pyrazol-4-yl)[1,2,4]triazolo[1,5-c]quinazolin-5(6H)-one

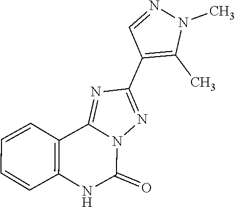 [SMILES: Cc1c(-c2nc3c4ccccc4[nH]c(=O)n3n2)cnn1C]

Methyl (2-cyanophenyl)carbamate (229 mg, 1.30 mmol) and 1,5-dimethyl-1H-pyrazole-4-carbohydrazide (CAS 864948-68-5, 200 mg, 1.30 mmol) were stirred in N,N-dimethylformamide (4.6 mL) at 120° C. for 20 hours. Water was added to the mixture, precipitated product was filtered off, washed with water and dried under reduced pressure at 60° C. to give 287 mg (80% purity, 63% yield) of the title compound.

LC-MS (Method 2): Rₜ=0.52 min; MS (ESIpos): m/z=281 [M+H]⁺

¹H-NMR (400 MHz, DMSO-d6) δ[ppm]: 2.518 (2.95), 2.523 (1.96), 2.707 (15.66), 2.727 (0.94), 2.888 (1.08), 3.757 (4.57), 3.829 (16.00), 7.090 (0.60), 7.371 (0.92), 7.373 (1.03), 7.391 (1.90), 7.409 (1.20), 7.411 (1.33), 7.423 (1.90), 7.444 (2.14), 7.674 (1.11), 7.678 (1.14), 7.693 (1.11), 7.696 (1.54), 7.698 (1.13), 7.713 (0.85), 7.717 (0.85), 7.934 (5.92), 7.969 (0.42), 8.177 (1.54), 8.181 (1.61), 8.198 (1.58), 8.200 (1.47).

### Intermediate 59

### 5-chloro-2-(1,5-dimethyl-1H-pyrazol-4-yl)[1,2,4]triazolo[1,5-c]quinazoline

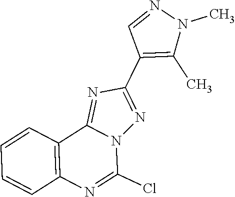 [SMILES: Cc1c(-c2nc3c4ccccc4nc(Cl)n3n2)cnn1C]

2-(1,5-Dimethyl-1H-pyrazol-4-yl)[1,2,4]triazolo[1,5-c]quinazolin-5(6H)-one (287 mg, 1.02 mmol) was solubilised in phosphorus(V) oxychloride (2.9 mL, 31 mmol), N,N-diisopropylethylamine (1.8 mL, 10 mmol) was added carefully and the mixture was stirred overnight at 110° C. The mixture was poured into ice and stirred for two hours. Precipitated product was filtered off, washed with water and dried at 60° C. under reduced pressure to give 237 mg (50% purity, 39% yield) of the title compound. The compound was used without purification.

LC-MS (Method 2): Rₜ=1.10 min; MS (ESIpos): m/z=299 [M+H]⁺

¹H-NMR (400 MHz, DMSO-d6): δ [ppm]=2.70 (s, 3H), 3.83 (s, 3H), 7.84 (td, 1H), 7.96 (dd, 1H), 8.01 (s, 1H), 8.02 (d, 1H), 8.47 (dd, 1H).

### Intermediate 60

### 2-(1H-pyrazol-4-yl)[1,2,4]triazolo[1,5-c]quinazolin-5(6H)-one

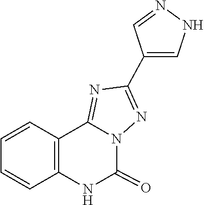 [SMILES: O=c1[nH]c2ccccc2c2nc(-c3cn[nH]c3)nn12]

Methyl (2-cyanophenyl)carbamate (500 mg, 2.84 mmol) and 1H-pyrazole-4-carbohydrazide (358 mg, 2.84 mmol) were stirred in DMF (10 mL) overnight at 120° C. 1H-pyrazole-4-carbohydrazide (358 mg, 2.84 mmol) was added and the reaction was stirred again overnight at 120° C. The reaction mixture was cooled to rt and diluted with water. The solid was filtered, washed with water and dried under reduced pressure at 60° C. to give 430 mg (60% yield) of the title compound.

LC-MS (method 2): Rₜ=0.46 min; MS (ESIpos): m/z=253 [M+H]⁺

¹H-NMR (400 MHz, DMSO-d6): δ [ppm]=7.35-7.48 (m, 2H), 7.70 (ddd, 1H), 8.09 (br s, 1H), 8.18 (dd, 1H), 8.44 (br s, 1H), 12.25 (br s, 1H), 13.31 (br s, 1H).

### Intermediate 61

### 5-chloro-2-(1H-pyrazol-4-yl)[1,2,4]triazolo[1,5-c]quinazoline

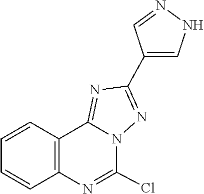 [SMILES: Clc1nc2ccccc2c2nc(-c3cn[nH]c3)nn12]

2-(1H-pyrazol-4-yl)[1,2,4]triazolo[1,5-c]quinazolin-5(6H)-one (215 mg, 852 μmol) was solubilised in phosphorus(V) oxychloride (2.8 mL, 30 mmol), N,N-diisopropylethylamine (1.5 mL, 8.5 mmol) was added carefully and the mixture was stirred overnight at 110° C. The mixture was carefully poured into ice and stirred for 1 h. The solid was filtered off, washed with water and dried at 60° C. under reduced pressure to give 208 mg (90% yield) of the title compound. The compound was used without further purification

LC-MS (method 2): Rₜ=0.87 min; MS (ESIpos): m/z=271 [M+H]⁺

¹H-NMR (400 MHz, DMSO-d₆): δ [ppm]=7.84 (ddd, 1H), 7.93-7.99 (m, 1H), 8.00-8.04 (m, 1H), 8.35 (s, 2H), 8.47 (dd, 1H).

### Intermediate 62

### 2-chloro-4-hydrazinoquinazoline

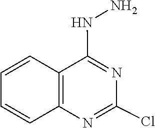 [SMILES: NNc1nc(Cl)nc2ccccc12]

2,4-Dichloroquinazoline (1 g, 5.02 mmol) was dissolved in THF (20 mL). Hydrazine hydrate (293 μL, 6.03 mmol) and triethylamine (2.52 mL, 18.09 mmol) were added. As the reaction mixture became viscous more THF (10 mL) was added. The reaction mixture was stirred overnight at rt. To the reaction mixture were added water (50 mL) and ethyl acetate (50 mL). The layers were separated and the aqueous phase was extracted two time with ethyl acetate (15 mL). The combined organic phases were washed with aqueous saturated ammonium chloride solution, dried over magnesium sulfate and concentrated under vacuum to afford 925 mg of the title compound which was used without further purification

LC-MS (method 2): Rₜ=0.60 min; MS (ESIpos): m/z=195 [M+H]⁺

¹H-NMR (400 MHz, DMSO-d₆): δ [ppm]=4.84 (br s, 2H), 7.46-7.51 (m, 1H), 7.60 (d, 1H), 7.73-7.80 (m, 1H), 8.18 (br d, 1H), 10.12 (br s, 1H).

### Intermediate 63

### N′-(2-chloroquinazolin-4-yl)-2-methyl-1,3-oxazole-4-carbohydrazide

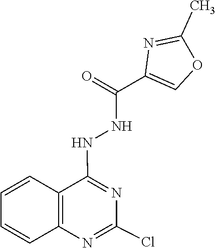 [SMILES: Cc1nc(C(=O)NNc2nc(Cl)nc3ccccc23)co1]

To a stirred solution of 2-methyl-1,3-oxazole-4-carboxylic acid (5 g, 39.3 mmol) in DMF (75 mL), N,N-diisopropylethylamine (10.15 g, 78.6 mmol) was added and the reaction mixture was cooled to 0° C. HATU (22.4 g, 58.9 mmol) was added followed by addition of 2-chloro-4-hydrazinoquinazoline (7.63 g, 0.125 mol). The reaction mixture was stirred for one hour and quenched with ice. The precipitate was filtered and washed with water and petroleum ether to afford the title compound. The crude material was used without further purification.

### Intermediate 64

### 2-(2-Methyl-1,3-oxazol-4-yl)[1,2,4]triazolo[1,5-c]quinazolin-5(6H)-one

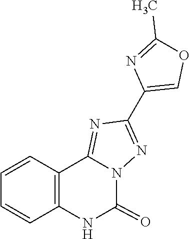 [SMILES: Cc1nc(-c2nc3c4ccccc4[nH]c(=O)n3n2)co1]

N′-(2-chloroquinazolin-4-yl)-2-methyl-1,3-oxazole-4-carbohydrazide (8.2 g, 28.7 mmol) was stirred in acetic acid (82 mL) at reflux for 6 h. The reaction mixture was then cooled to rt and diluted with ice cold water. The precipitate was filtered and washed with water and petroleum ether to afford the title compound. The crude material was used without further purification.

### Intermediate 65

### 5-Chloro-2-(2-methyl-1,3-oxazol-4-yl)[1,2,4]triazolo[1,5-c]quinazoline

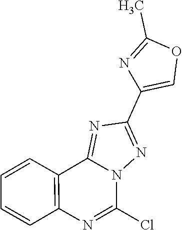 [SMILES: Cc1nc(-c2nc3c4ccccc4nc(Cl)n3n2)co1]

2-(2-Methyl-1,3-oxazol-4-yl)[1,2,4]triazolo[1,5-c]quinazolin-5(6H)-one (3.3 g, 12.3 mmol) and N, N-diisopropylethylamine ((9.53 g, 74.0 mmol) were stirred four hours in POCl₃ (114 mL) at reflux. The reaction was cooled to rt and diluted with DCM and ice cold water. The aqueous phase was extracted with DCM. The organic phase was dried (Na₂SO₄) filtered and concentrated under reduced pressure to give the title compound without further purification.

### Intermediate 66

### 2-(4-fluorophenyl)[1,2,4]triazolo[1,5-c]quinazolin-5(6H)-one

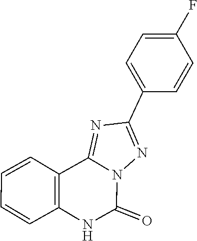 [SMILES: O=c1[nH]c2ccccc2c2nc(-c3ccc(F)cc3)nn12]

Methyl (2-cyanophenyl)carbamate (500 mg, 2.84 mmol) and 4-fluorobenzohydrazide (CAS 456-06-4, 437 mg, 2.84 mmol) were stirred in N,N-dimethylformamide (10 mL) at 120° C. for 20 hours. Water was added to the mixture, precipitated product was filtered off, washed with water and dried under reduced pressure at 60° C. to give 725 mg (96% purity, 88% yield) of the title compound.

LC-MS (Method 2): Rₜ=0.70 min; MS (ESIpos): m/z=281 [M+H]⁺

¹H-NMR (400 MHz, DMSO-d6): δ [ppm]=7.38-7.48 (m, 4H), 7.72 (ddd, 1H), 8.20-8.30 (m, 3H), 12.35 (br s, 1H).

### Intermediate 67

### 5-chloro-2-(4-fluorophenyl)[1,2,4]triazolo[1,5-c]quinazoline

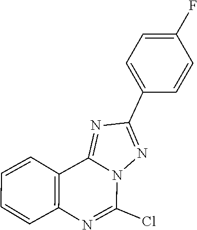 [SMILES: Fc1ccc(-c2nc3c4ccccc4nc(Cl)n3n2)cc1]

2-(4-Fluorophenyl)[1,2,4]triazolo[1,5-c]quinazolin-5(6H)-one (725 mg, 2.59 mmol) was solubilised in phosphorus(V) oxychloride (8.0 mL, 86 mmol), N,N-diisopropylethylamine (4.5 mL, 26 mmol) was added carefully and the mixture was stirred overnight at 110° C. The mixture was poured into ice and stirred for one hour. The organic solvent was evaporated and the solid was filtered off, washed with water and dried at 60° C. under reduced pressure to give 725 mg (99% purity, 93% yield) of the title compound.

LC-MS (Method 2): Rₜ=1.41 min; MS (ESIpos): m/z=299 [M+H]⁺

¹H-NMR (400 MHz, DMSO-d6): δ [ppm]=7.41-7.48 (m, 2H), 7.87 (ddd, 1H), 7.96-8.02 (m, 1H), 8.03-8.07 (m, 1H), 8.31-8.38 (m, 2H), 8.49-8.55 (m, 1H).

### Intermediate 68

### 2-(3-methoxyphenyl)[1,2,4]triazolo[1,5-c]quinazolin-5(6H)-one

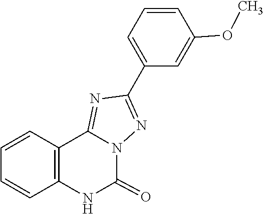 [SMILES: COc1cccc(-c2nc3c4ccccc4[nH]c(=O)n3n2)c1]

Methyl (2-cyanophenyl)carbamate (2.00 g, 11.4 mmol) and 3-methoxybenzohydrazide (CAS 5785-06-8, 2.26 g, 13.6 mmol) were stirred in N-methylpyrrolidone (50 mL) at 120° C. for 4 hours. Water was added to the mixture, precipitated product was filtered off, washed with water and dried under reduced pressure at 60° C. to give 2.63 g (93% purity, 74% yield) of the title compound.

LC-MS (Method 2): Rₜ=0.64 min; MS (ESIpos): m/z=293 [M+H]⁺

¹H-NMR (400 MHz, DMSO-d6): δ [ppm]=3.88 (s, 3H), 7.12 (ddd, 1H), 7.38-7.53 (m, 3H), 7.69-7.75 (m, 2H), 7.83 (dt, 1H), 8.24 (dd, 1H), 12.35 (s, 1H)

### Intermediate 69

### 5-chloro-2-(3-methoxyphenyl)[1,2,4]triazolo[1,5-c]quinazoline

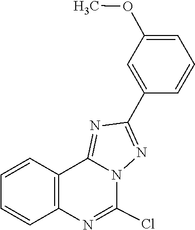 [SMILES: COc1cccc(-c2nc3c4ccccc4nc(Cl)n3n2)c1]

2-(3-Methoxyphenyl)[1,2,4]triazolo[1,5-c]quinazolin-5(6H)-one (2.63 g, 8.99 mmol) was solubilised in phosphorus(V) oxychloride (26 mL, 282 mmol), N,N-diisopropylethylamine (16 mL, 90 mmol) was added carefully and the mixture was stirred overnight at 110° C. The mixture was evaporated and the residue was diluted with ethyl acetate. Precipitated product was filtered off to give 2.86 g (100% purity, 100% yield) of the title compound.

LC-MS (Method 2): Rₜ=1.36 min; MS (ESIpos): m/z=311 [M+H]⁺

¹H-NMR (400 MHz, DMSO-d6): δ [ppm]=3.89 (s, 3H), 7.17 (ddd, 1H), 7.53 (t, 1H), 7.79 (dd, 1H), 7.84-7.92 (m, 2H), 7.98 (td, 1H), 8.03-8.08 (m, 1H), 8.53 (dd, 1H)

### Intermediate 70

### N²-(tert-butoxycarbonyl)-N-propan-2-yl-D-alaninamide

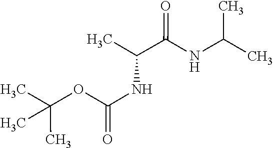 [SMILES: CC(C)NC(=O)[C@@H](C)NC(=O)OC(C)(C)C]

N-(tert-Butoxycarbonyl)-D-alanine (100 mg, 529 μmol), propan-2-amine (90 μL, 1.1 mmol), sodium hydrogen carbonate (133 mg, 1.59 mmol) and HATU (402 mg, 1.06 mmol) were stirred in dichloromethane (1.5 mL) overnight at rt. The solid was filtered and washed with DCM. The filtrate was diluted with water and extracted with DCM/MeOH (9/1). The organic layer was dried (silicone filter) and concentrated under reduced pressure to give 97 mg (100% purity, 80% yield) of the title compound without further purification.

¹H-NMR (400 MHz, DMSO-d6): δ [ppm]=1.03 (dd, 6H), 1.13 (d, 3H), 1.37 (s, 9H), 3.69-3.98 (m, 2H), 6.75 (br d, 1H), 7.56 (br d, 1H).

### Intermediate 71

### N²-(tert-butoxycarbonyl)-N-cyclopropyl-D-alaninamide

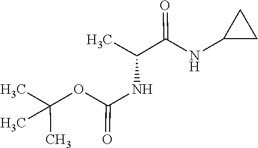 [SMILES: C[C@@H](NC(=O)OC(C)(C)C)C(=O)NC1CC1]

N-(tert-Butoxycarbonyl)-D-alanine (100 mg, 529 μmol), cyclopropanamine (60.4 mg, 1.06 mmol), sodium hydrogen carbonate (133 mg, 1.59 mmol) and HATU (402 mg, 1.06 mmol) were stirred in dichloromethane (1.5 mL) overnight at rt. The solid was filtered and washed with DCM. The mixture was diluted with water and extracted with DCM/MeOH (9/1). The organic layer was dried (silicone filter) and concentrated under reduced pressure to give 155 mg (100% purity, 96% yield) of the title compound without further purification.

¹H-NMR (400 MHz, DMSO-d6): δ [ppm]=0.34-0.40 (m, 2H), 0.58-0.61 (m, 2H), 0.75-0.82 (m, 1H), 1.11 (d, 3H), 1.36 (s, 9H), 3.79-3.90 (m, 1H), 6.77 (br d, 1H), 7.83 (br d, 1H).

### Intermediate 72

### N²-(tert-butoxycarbonyl)-N-ethyl-D-alaninamide

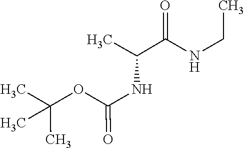

N-(tert-Butoxycarbonyl)-D-alanine (100 mg, 529 μmol), ethanamine (530 μL, 2.0 M in THF, 1.1 mmol), sodium hydrogen carbonate (133 mg, 1.59 mmol) and HATU (402 mg, 1.06 mmol) were stirred in DCM (1.5 mL) overnight at rt. The mixture was diluted with water and extracted with DCM/MeOH (9/1). The organic layer was dried (silicone filter) and concentrated under reduced pressure. to give 155 mg (100% purity, 94% yield) of the title compound without further purification.

¹H-NMR (400 MHz, DMSO-d6): δ [ppm]=0.99 (t, 3H), 1.14 (d, 3H), 1.37 (s, 9H), 3.01-3.09 (m, 2H), 3.88 (br t, 1H), 6.81 (br d, 1H), 7.73 (br s, 1H).

### Intermediate 73

### N²-(tert-butoxycarbonyl)-N-methyl-D-alaninamide

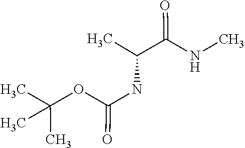

N-(tert-Butoxycarbonyl)-D-alanine (100 mg, 529 μmol), methanamine (530 μL, 2.0 M in THF, 1.1 mmol), sodium hydrogen carbonate (133 mg, 1.59 mmol) and HATU (402 mg, 1.06 mmol) were stirred in DCM (1.5 mL) overnight at rt. The mixture was diluted with water and extracted with DCM/MeOH (9/1). The organic layer was dried (silicone filter) and concentrated under reduced pressure. to give 110 mg (100% purity, 92% yield) of the title compound without further purification.

¹H-NMR (400 MHz, DMSO-d6): δ [ppm]=1.14 (d, 3H), 1.37 (s, 9H), 2.56 (d, 3H), 3.82-3.95 (m, 1H), 6.85 (br d, 1H), 7.70 (br d, 1H).

### Intermediate 74

### tert-butyl [(2R)-1-(cyclobutylamino)-1-oxopropan-2-yl]carbamate

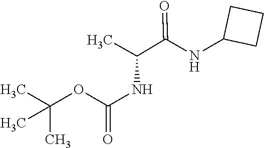 [SMILES: C[C@@H](NC(=O)OC(C)(C)C)C(=O)NC1CCC1]

N-(tert-Butoxycarbonyl)-D-alanine (100 mg, 529 μmol), cyclobutanamine (75.2 mg, 1.06 mmol), sodium hydrogen carbonate (133 mg, 1.59 mmol) and HATU (402 mg, 1.06 mmol) were stirred in DCM (1.5 mL) overnight at rt. The mixture was diluted with water and extracted with DCM/MeOH (9/1). The organic layer was dried (silicone filter) and concentrated under reduced pressure. to give 168 mg (75% purity, 98% yield) of the title compound without further purification.

¹H-NMR (400 MHz, DMSO-d6): δ [ppm]=1.12 (d, 3H), 1.37 (s, 9H), 1.54-1.69 (m, 2H), 1.77-1.96 (m, 2H), 2.06-2.19 (m, 2H), 3.87 (quin, 1H), 4.15 (sxt, 1H), 6.73-6.81 (m, 1H), 7.98 (br d, 1H).

### Intermediate 75

### N²-(tert-butoxycarbonyl)-N,N-dimethyl-D-alaninamide

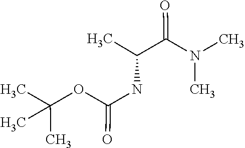

N-(tert-Butoxycarbonyl)-D-alanine (100 mg, 529 μmol), N-methylmethanamine (530 μL, 2.0 M in THF, 1.1 mmol), sodium hydrogen carbonate (133 mg, 1.59 mmol) and HATU (402 mg, 1.06 mmol) were stirred in DCM (1.5 mL) overnight at rt. The mixture was diluted with water and extracted with DCM/MeOH (9/1). The organic layer was dried (silicone filter) and concentrated under reduced pressure. to give 155 mg of the title compound without further purification.

¹H-NMR (400 MHz, DMSO-d6): δ [ppm]=1.11 (d, 3H), 1.36 (s, 9H), 2.81 (s, 3H), 2.99 (s, 3H), 4.40 (quin, 1H), 6.89 (br d, 1H).

### Intermediate 76

### N²-(tert-butoxycarbonyl)-N-(2-hydroxyethyl)-D-alaninamide

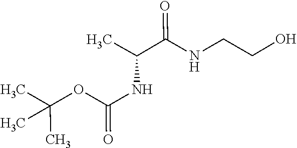 [SMILES: C[C@@H](NC(=O)OC(C)(C)C)C(=O)NCCO]

N-(tert-Butoxycarbonyl)-D-alanine (100 mg, 529 μmol), 2-aminoethan-1-ol (64.6 mg, 1.06 mmol), sodium hydrogen carbonate (133 mg, 1.59 mmol) and HATU (402 mg, 1.06 mmol) were stirred in DCM (1.5 mL) overnight at rt. The mixture was diluted with water and extracted with DCM/MeOH (9/1). The aqueous phase was lyophilized to give 600 mg of the crude title compound that was used without further purification.

### Intermediate 77

### N²-(tert-butoxycarbonyl)-N-(3-hydroxypropyl)-D-alaninamide

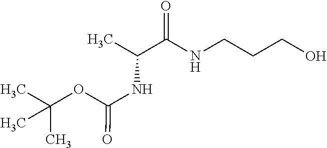 [SMILES: C[C@@H](NC(=O)OC(C)(C)C)C(=O)NCCCO]

N-(tert-Butoxycarbonyl)-D-alanine (100 mg, 529 μmol), 3-aminopropan-1-ol (79.4 mg, 1.06 mmol), sodium hydrogen carbonate (133 mg, 1.59 mmol) and HATU (402 mg, 1.06 mmol) were stirred in DCM (1.5 mL) overnight at rt. The mixture was diluted with water and extracted with DCM/MeOH (9/1). The aqueous layer was lyophilized to give 58 mg of the crude title compound that was used without further purification.

### Intermediate 78

### N-propan-2-yl-D-alaninamide hydrochloride

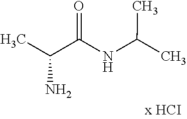

N²-(tert-Butoxycarbonyl)-N-propan-2-yl-D-alaninamide (97.0 mg, 421 μmol) was solubilised in dichloromethane (5.6 mL) and methanol (1.4 mL). HCl (1.6 mL, 4.0 M in dioxane, 6.3 mmol) was added and the mixture was stirred overnight at rt. The reaction mixture was concentrated under reduced pressure to give 97 mg of the title compound. The compound was used without further purification.

### Intermediate 79

### N-cyclopropyl-D-alaninamide hydrochloride

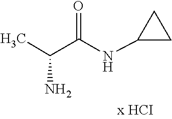

N²-(tert-Butoxycarbonyl)-N-cyclopropyl-D-alaninamide (155 mg, 766 μmol) was dissolved in dichloromethane (5.0 mL) and methanol (2.0 mL), HCl (2.9 mL, 4.0 M in dioxane, 11 mmol) was added and the mixture was stirred overnight at rt. The reaction mixture was concentrated under reduced pressure to give 116 mg of the title product that was used without further purification.

### Intermediate 80

### N-ethyl-D-alaninamide hydrochloride

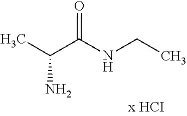

N²-(tert-Butoxycarbonyl)-N-ethyl-D-alaninamide (155 mg, 766 μmol) was dissolved in dichloromethane (5.0 mL) and methanol (2.0 mL), HCl (2.9 mL, 4.0 M in dioxane, 11 mmol) was added and the mixture was stirred overnight at rt. The reaction mixture was concentrated under reduced pressure to give 101 mg of the title compound that was used without further purification.

### Intermediate 81

### N-methyl-D-alaninamide hydrochloride

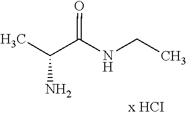

N²-(tert-Butoxycarbonyl)-N-methyl-D-alaninamide (110 mg, 544 μmol) was dissolved in dichloromethane (5.0 mL) and methanol (2.0 mL), HCl (2.0 mL, 4.0 M in dioxane, 8.2 mmol) was added and the mixture was stirred overnight at rt. The reaction mixture was concentrated under reduced pressure to give 70 mg of the title compound that was used without further purification.

### Intermediate 82

### N-cyclobutyl-D-alaninamide hydrochloride

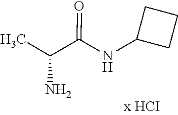

tert-butyl [(2R)-1-(cyclobutylamino)-1-oxopropan-2-yl]carbamate (168 mg, 75% purity, 520 μmol) was dissolved in dichloromethane (4.8 mL) and methanol (1.9 mL), HCl (1.9 mL, 4.0 M in dioxane, 7.8 mmol) was added and the mixture was stirred overnight at rt. The reaction mixture was concentrated under reduced pressure to give 158 mg of the title compound that was used without further purification.

### Intermediate 83

### N,N-dimethyl-D-alaninamide hydrochloride

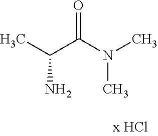

N²-(tert-Butoxycarbonyl)-N,N-dimethyl-D-alaninamide (153 mg, 70% purity, 495 μmol) was dissolved in dichloromethane (5.0 mL) and methanol (1.5 mL), HCl (1.9 mL, 4.0 M in dioxane, 7.4 mmol) was added and the mixture was stirred overnight at rt. The reaction mixture was concentrated under reduced pressure to give 133 mg of the title compound that was used without further purification.

### Intermediate 84

### N-(2-hydroxyethyl)-D-alaninamide hydrochloride

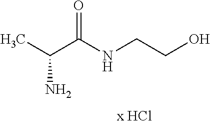

N²-(tert-butoxycarbonyl)-N-(2-hydroxyethyl)-D-alaninamide (600 mg, 20% purity, 517 μmol) was dissolved in dichloromethane (4.7 mL) and methanol (1.9 mL), HCl (1.9 mL, 4.0 M in dioxane, 7.7 mmol) was added and the mixture was stirred overnight at rt. The reaction mixture was concentrated under reduced pressure to give 90 mg of the title compound that was used without further purification.

### Intermediate 85

### N-(3-hydroxypropyl)-D-alaninamide hydrochloride

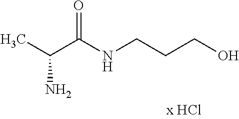

N²-(tert-Butoxycarbonyl)-N-(3-hydroxypropyl)-D-alaninamide (58.0 mg, 235 μmol) was dissolved in dichloromethane (2.2 mL) and methanol (870 μl), HCl (880 μL, 4.0 M in dioxane, 3.5 mmol) was added and the mixture was stirred overnight at rt. The reaction mixture was concentrated under reduced pressure to give 45 mg of the title compound that was used without further purification.

### Intermediate 86

### 2-(pyridin-2-yl)[1,2,4]triazolo[1,5-c]quinazoline-5(6H)-thione

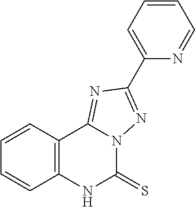 [SMILES: S=c1[nH]c2ccccc2c2nc(-c3ccccn3)nn12]

2-Isothiocyanatobenzonitrile (CAS 81431-98-3, 500 mg, 3.12 mmol) and pyridine-2-carbohydrazide (CAS 1452-63-7, 428 mg, 3.12 mmol) were solubilised in 100 mL ethanol and the reaction mixture was stirred at reflux overnight. Precipitated product was filtered off, washed with ethanol and dried at 40° C. to give 769 mg (90% purity, 79% yield) of the title compound.

LC-MS (Method 2): Rₜ=0.50 min; MS (ESIpos): m/z=280 [M+H]⁺

¹H-NMR (400 MHz, DMSO-d6): δ [ppm]=7.54-7.61 (m, 2H), 7.69 (d, 1H), 7.78-7.85 (m, 1H), 8.04 (td, 1H), 8.29-8.35 (m, 2H), 8.77-8.82 (m, 1H), 14.08 (br s, 1H).

### Intermediate 87

### 2-(pyridin-3-yl)[1,2,4]triazolo[1,5-c]quinazoline-5(6H)-thione

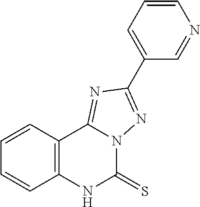 [SMILES: S=c1[nH]c2ccccc2c2nc(-c3cccnc3)nn12]

2-Isothiocyanatobenzonitrile (CAS 81431-98-3, 500 mg, 3.12 mmol) and pyridine-3-carbohydrazide (CAS 553-53-7, 428 mg, 3.12 mmol) were solubilised in ethanol (100 mL) and the mixture was stirred at reflux overnight. The reaction was filtered and washed with ethanol to give 754 mg (95% purity, 82% yield) of the title compound.

LC-MS (Method 2): Rₜ=0.49 min; MS (ESIpos): m/z=280 [M+H]⁺

¹H-NMR (400 MHz, DMSO-d6): δ [ppm]=7.53-7.59 (m, 1H), 7.64 (ddd, 1H), 7.69 (d, 1H), 7.78-7.86 (m, 1H), 8.32 (dd, 1H), 8.57 (dt, 1H), 8.77 (dd, 1H), 9.40 (dd, 1H), 14.09 (br s, 1H).

### Intermediate 88

### 2-(pyridin-4-yl)[1,2,4]triazolo[1,5-c]quinazoline-5(6H)-thione

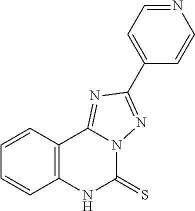 [SMILES: S=c1[nH]c2ccccc2c2nc(-c3ccncc3)nn12]

2-Isothiocyanatobenzonitrile (250 mg, 1.56 mmol) and pyridine-4-carbohydrazide (214 mg, 1.56 mmol) were solubilised in ethanol (50 mL) and the mixture was stirred at reflux overnight. The reaction was filtered, the solid was washed with ethanol and dried under reduced pressure to give 367 mg (100% purity, 84% yield) of the title compound.

¹H-NMR (400 MHz, DMSO-d6): δ [ppm]=7.53-7.60 (m, 1H), 7.69 (d, 1H), 7.80-7.86 (m, 1H), 8.13-8.18 (m, 2H), 8.31 (dd, 1H), 8.80-8.84 (m, 2H), 14.12 (br s, 1H).

### Intermediate 89

### 2-(pyridazin-4-yl)[1,2,4]triazolo[1,5-c]quinazoline-5(6H)-thione

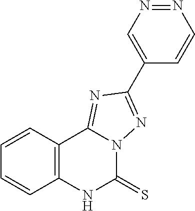 [SMILES: S=c1[nH]c2ccccc2c2nc(-c3ccnnc3)nn12]

2-Isothiocyanatobenzonitrile (CAS 81431-98-3, 100 mg, 624 μmol) and pyridazine-4-carbohydrazide (CAS 56932-26-4, 86.2 mg, 624 μmol) were solubilised in ethanol (2 mL) and the reaction mixture was stirred at reflux overnight. Precipitated product was filtered off, washed with ethanol and dried to give 138 mg (67% purity, 53% yield) of the title compound.

LC-MS (Method 1): Rₜ=0.71 min; MS (ESIpos): m/z=281 [M+H]⁺

### Intermediate 90

### 2-[4-(dimethylamino)phenyl][1,2,4]triazolo[1,5-c]quinazoline-5(6H)-thione

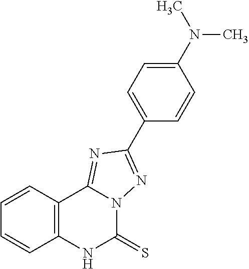 [SMILES: CN(C)c1ccc(-c2nc3c4ccccc4[nH]c(=S)n3n2)cc1]

2-Isothiocyanatobenzonitrile (CAS 81431-98-3, 186 mg, 1.16 mmol) and 4-(dimethylamino)benzohydrazide (CAS 19353-92-5, 208 mg, 1.16 mmol) were solubilised in ethanol (37 mL) and the reaction mixture was stirred at reflux overnight. Precipitated product was filtered off, washed with ethanol and dried to give 288 mg (86% purity, 66% yield) of the title compound.

LC-MS (Method 2): Rₜ=0.62 min; MS (ESIpos): m/z=322 [M+H]⁺

¹H-NMR (400 MHz, DMSO-d6) δ[ppm]: 1.035 (0.39), 1.052 (0.67), 1.070 (0.41), 2.518 (0.77), 2.523 (0.50), 2.948 (0.51), 2.997 (0.77), 3.014 (9.62), 3.020 (16.00), 6.767 (0.97), 6.789 (0.96), 6.843 (0.20), 6.850 (1.84), 6.855 (0.57), 6.867 (0.60), 6.872 (1.91), 6.879 (0.21), 7.251 (0.23), 7.270 (0.42), 7.287 (0.60), 7.306 (0.49), 7.506 (0.41), 7.508 (0.43), 7.526 (0.83), 7.544 (0.48), 7.546 (0.49), 7.635 (0.19), 7.651 (0.80), 7.671 (1.01), 7.761 (0.57), 7.765 (0.57), 7.779 (0.55), 7.782 (0.75), 7.785 (0.50), 7.800 (0.39), 7.804 (0.37), 7.872 (0.45), 7.893 (0.43), 8.052 (0.23), 8.059 (2.30), 8.064 (0.62), 8.077 (0.62), 8.082 (2.11), 8.089 (0.26), 8.098 (0.23), 8.118 (0.21), 8.261 (0.75), 8.264 (0.78), 8.281 (0.73), 8.284 (0.68), 10.864 (0.29).

### Intermediate 94

### 2-(furan-2-yl)[1,2,4]triazolo[1,5-c]quinazolin-5(6H)-one

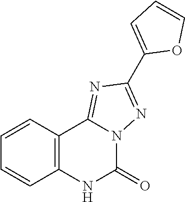 [SMILES: O=c1[nH]c2ccccc2c2nc(-c3ccco3)nn12]

Methyl (2-cyanophenyl)carbamate (200 mg, 1.14 mmol) and furan-2-carbohydrazide (143 mg, 1.14 mmol) were stirred in DMF (4.0 mL) at 120° C. for 20 h. The reaction was diluted with water and the mixture was filtered. The solid was washed with water and dried under reduced pressure at 60° C. to give 247 mg (80% purity, 69% yield) of the title compound.

LC-MS (Method 2): Rₜ=0.55 min; MS (ESIpos): m/z=253 [M+H]⁺

¹H-NMR (400 MHz, DMSO-d6): δ [ppm]=6.74 (dd, 1H), 7.25 (dd, 1H), 7.38-7.43 (m, 1H), 7.45 (d, 1H), 7.72 (ddd, 1H), 7.96 (dd, 1H), 8.19 (dd, 1H). (one proton is not visible.)

### Intermediate 95

### 5-chloro-2-(furan-2-yl)[1,2,4]triazolo[1,5-c]quinazoline

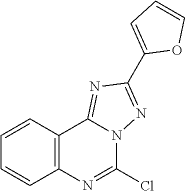 [SMILES: Clc1nc2ccccc2c2nc(-c3ccco3)nn12]

2-(Furan-2-yl)[1,2,4]triazolo[1,5-c]quinazolin-5(6H)-one (247 mg, 981 μmol) was solubilised in phosphorus(V) oxychloride (4.4 mL, 48 mmol), N,N-diisopropylethylamine (1.7 mL, 9.8 mmol) was added carefully and the mixture was stirred for 5 h at 110° C. The mixture was cooled to rt and concentrated under reduced pressure. The crude material was solubilized in DCM, poured into ice and stirred for 10 min. The solid was filtered, washed with water and dried under reduced pressure at 60° C. to give 196 mg (80% purity, 59% yield) of the title compound without further purification.

LC-MS (method 2): Rₜ=1.15 min; MS (ESIpos): m/z=271 [M+H]⁺

¹H-NMR (400 MHz, DMSO-d6): δ [ppm]=6.78 (dd, 1H), 7.38 (dd, 1H), 7.86 (ddd, 1H), 7.96-8.07 (m, 3H), 8.46-8.52 (m, 1H).

### Intermediate 96

### ethyl [2-(2-chloroquinazolin-4-yl)hydrazino](oxo)acetate

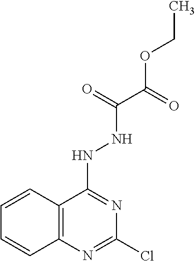 [SMILES: CCOC(=O)C(=O)NNc1nc(Cl)nc2ccccc12]

To a stirred suspension of 2-chloro-4-hydrazinylquinazoline (200 mg, 1.03 mmol) in dichloromethane (2 mL) was added triethylamine (0.43 mL, 3.08 mmol). At −5° C. ethyl chloro(oxo)acetate (132 μL, 1.18 mmol) was added dropwise. The reaction mixture was stirred at −5° C. to 0° C. for 1 hour. The precipitate was filtered off and washed with a small volume of water. The precipitate in filtrate was filtered again but the second filtrate and the second precipitate were combined and the pH was adjusted to 6 with hydrochloric acid (0.5M HCl, 0.6 mL). Dichloromethane (40 mL) was added, the layers were separated and the aqueous phase was extracted with dichloromethane. The combined organic phases were washed twice with water, dried over magnesium sulfate and concentrated giving 138 mg of the title compound which contained ca. 0.35 mole of trimethylamine and was used without purification in the next step.

LC-MS (method 1): Rₜ=0.81 min; MS (ESIpos): m/z=295 [M+H]⁺

¹H-NMR (400 MHz, DMSO-d₆): δ [ppm]=1.32 (t, 3H), 4.33 (q, 2H), 7.49-7.65 (m, 2H), 7.79 (br s, 1H), 8.20 (br d, 1H), 10.16 (br s, 2H).

### Intermediate 97

### ethyl 5-oxo-5,6-dihydro[1,2,4]triazolo[1,5-c]quinazoline-2-carboxylate

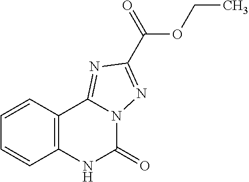 [SMILES: CCOC(=O)c1nc2c3ccccc3[nH]c(=O)n2n1]

Ethyl [2-(2-chloroquinazolin-4-yl)hydrazino](oxo)acetate (13.6 g, 46.0 mmol) was stirred in acetic acid (136 mL) for 6 h at reflux. The reaction mixture was then cooled to rt and diluted with ice cold water. The precipitate was filtered and the solid was washed with water and petroleum ether to give the title compound without further purification.

### Intermediate 98

### ethyl 5-chloro[1,2,4]triazolo[1,5-c]quinazoline-2-carboxylate

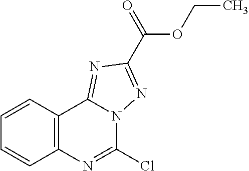 [SMILES: CCOC(=O)c1nc2c3ccccc3nc(Cl)n2n1]

Ethyl 5-oxo-5,6-dihydro[1,2,4]triazolo[1,5-c]quinazoline-2-carboxylate (11.4 g, 46.4 mmol) and N, N-diisopropylethylamine (36.0 g, 278 mmol) were stirred in POCl₃ (114 mL). The reaction mixture was heated to reflux for 4 h. The reaction mixture was cooled to rt and concentrated under reduced pressure. The crude mixture was diluted with DCM (200 mL) and ice cold water. The aqueous phase extracted with dichloromethane and the combined organic phase was dried (sodium sulphate), filtered and concentrated under reduced pressure to give the title compound without further purification.

### Intermediate 99

### ethyl 5-{[(3R)-2-oxoazepan-3-yl]amino}[1,2,4]triazolo[1,5-c]quinazoline-2-carboxylate

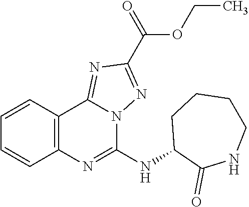 [SMILES: CCOC(=O)c1nc2c3ccccc3nc(N[C@@H]3CCCCNC3=O)n2n1]

Ethyl 5-chloro[1,2,4]triazolo[1,5-c]quinazoline-2-carboxylate (500 mg, 1.81 mmol), (3R)-3-aminoazepan-2-one (255 mg, 1.99 mmol) and N,N-diisopropylethylamine (630 μL, 3.6 mmol) were stirred in DMF (8.1 mL) for 2 h at 60° C. The reaction was cooled to rt and diluted with water. The solid was filtered, washed with water and dried under reduced pressure at 60° C. to give 486 mg (73% yield) of the title compound.

LC-MS (method 2): Rₜ=1.08 min; MS (ESIpos): m/z=369 [M+H]⁺

¹H-NMR (400 MHz, DMSO-d6): δ [ppm]=1.26-1.35 (m, 1H), 1.39 (t, 3H), 1.48-1.61 (m, 1H), 1.79-1.94 (m, 2H), 1.97-2.06 (m, 1H), 2.26-2.35 (m, 1H), 3.10-3.21 (m, 1H), 3.30-3.41 (m, 1H), 4.47 (q, 2H), 4.82 (dd, 1H), 7.48 (ddd, 1H), 7.66-7.71 (m, 1H), 7.74-7.80 (m, 2H), 8.24 (dd, 1H), 8.29 (dd, 1H).

### Intermediate 100

### 7-fluoro-2-(3-fluorophenyl)[1,2,4]triazolo[1,5-c]quinazolin-5(6H)-one

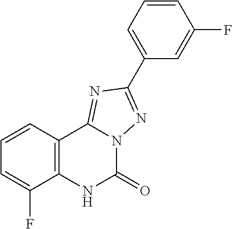 [SMILES: O=c1[nH]c2c(F)cccc2c2nc(-c3cccc(F)c3)nn12]

Ethyl (2-cyano-6-fluorophenyl)carbamate (200 mg, 961 μmol) and 3-fluorobenzohydrazide (178 mg, 1.15 mmol) were stirred in DMF (2.1 mL) overnight at 120° C. The reaction was cooled to rt and diluted with water. The solid was filtered, washed with water and dried under reduced pressure at 60° C. to give 240 mg (84% yield) of the title compound without further purification.

LC-MS (Method 2): Rₜ=0.64 min; MS (ESIpos): m/z=299 [M+H]⁺

The following intermediates were prepared similarly:

| | Structure |
|---|---|
| | IUPAC-Name |
| | LC-MS (method): Retention time; Mass found |
| Intermediate | ¹H-NMR |
| Intermediate 101 | 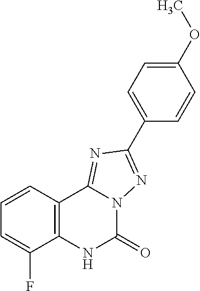 [SMILES: COc1ccc(-c2nc3c4cccc(F)c4[nH]c(=O)n3n2)cc1] |
| | 7-fluoro-2-(4-methoxyphenyl)[1,2,4] |
| | triazolo[1,5-c]quinazolin-5(6H)-one |
| | LC-MS (Method 2): Rt = 0.62 min; |
| | MS (ESIpos): m/z = 311 [M + H]⁺ |
| Intermediate 102 | 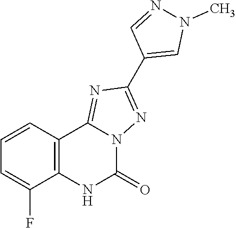 [SMILES: Cn1cc(-c2nc3c4cccc(F)c4[nH]c(=O)n3n2)cn1] |
| | 7-fluoro-2-(1-methyl-1H-pyrazol-4-yl) |
| | [1,2,4]triazolo[1,5-c]quinazolin-5(6H)-one |
| | LC-MS (Method 2): Rt = 0.47 min; |
| | MS (ESIpos): m/z = 285 [M + H]⁺ |
| Intermediate 103 | 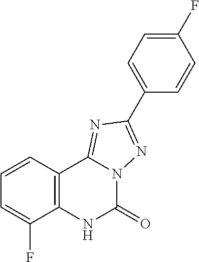 [SMILES: O=c1[nH]c2c(F)cccc2c2nc(-c3ccc(F)cc3)nn12] |
| | 7-fluoro-2-(4-fluorophenyl)[1,2,4] |
| | triazolo[1,5-c]quinazolin-5(6H)-one |
| | LC-MS (Method 2): Rt = 0.63 min; |
| | MS (ESIpos): m/z = 299 [M + H]⁺ |
| Intermediate 104 | 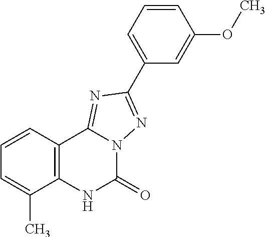 [SMILES: COc1cccc(-c2nc3c4cccc(C)c4[nH]c(=O)n3n2)c1] |
| | 2-(3-methoxyphenyl)-7-methyl[1,2,4] |
| | triazolo[1,5-c]quinazolin-5(6H)-one |
| | LC-MS (Method 2): Rₜ = 0.70 min; |
| | MS (ESIpos): m/z = 307 [M + H]⁺ |
| | ¹H-NMR (400 MHz, DMSO-d₆): δ |
| | [ppm] = 3.88 (s, 3H), 7.12 (ddd, 1H), 7.33 (t, |
| | 1H), 7.50 (t, 1H), 7.56 (d, 1H), |
| | 7.74 (dd, 1H), 7.83 (dt, 1H), 8.09-8.14 (m, |
| | 1H), 11.53 (brs, 1H). |
| Intermediate 105 | 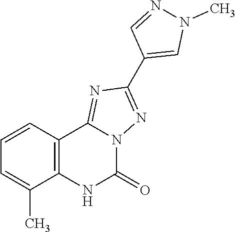 [SMILES: Cc1cccc2c1[nH]c(=O)n1nc(-c3cnn(C)c3)nc21] |
| | 7-methyl-2-(1-methyl-1H-pyrazol-4-yl) |
| | [1,2,4]triazolo[1,5-c]quinazolin-5(6H)-one |
| | LC-MS (Method 2): Rₜ = 0.53 min; |
| | MS (ESIpos): m/z = 281 [M + H]⁺ |
| | ¹H-NMR (400 MHz, DMSO-d₆): δ |
| | [ppm] = 3.94 (s, 3H), 7.31 (t, 1H), 7.51- |
| | 7.57 (m, 1H), 8.01 (d, 1H), 8.04 (dd, |
| | 1H), 8.41 (s, 1H), 11.43 (s, 1H). |
| Intermediate 106 | 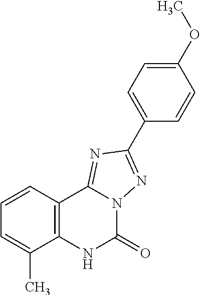 [SMILES: COc1ccc(-c2nc3c4cccc(C)c4[nH]c(=O)n3n2)cc1] |
| | 2-(4-methoxyphenyl)-7-methyl[1,2,4] |
| | triazolo[1,5-c]quinazolin-5(6H)-one |
| | LC-MS (Method 2): Rₜ = 0.71 min; |
| | MS (ESIpos): m/z = 307 [M + H]⁺ |
| | ¹H-NMR (400 MHz, DMSO-d₆): δ |
| | [ppm] = 3.85 (s, 3H), 7.10-7.15 (m, 2H), |
| | 7.32 (t, 1H), 7.55 (d, 1H), 8.09 (d, 1H), |
| | 8.14-8.19 (m, 2H), 11.47 (brs, 1H). |
| Intermediate 107 | 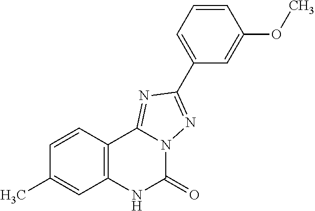 |
| | 2-(3-methoxyphenyl)-8-methyl[1,2,4] |
| | triazolo[1,5-c]quinazolin-5(6H)-one |
| | LC-MS (Method 2): Rₜ = 0.73 min; |
| | MS (ESIneg): m/z = 305 [M − H]⁻ |
| | ¹H-NMR (400 MHz, DMSO-d₆): δ |
| | [ppm] = 2.44 (s, 3H), 3.87 (s, 3H), 7.11 |
| | (ddd, 1H), 7.20-7.27 (m, 2H), 7.48 (t, 1H), |
| | 7.71 (dd, 1H), 7.81 (dt, 1H), 8.11 |
| | (d, 1H), 12.28 (br s, 1H). |
| Intermediate 108 | 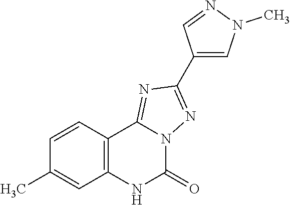 |
| | 8-methyl-2-(1-methyl-1H-pyrazol-4-yl) |
| | [1,2,4]triazolo[1,5-c]quinazolin-5(6H)-one |
| | LC-MS (Method 2): Rt = 0.57 min; |
| | MS (ESIpos): m/z = 281 [M + H]⁺ |
| Intermediate 109 | 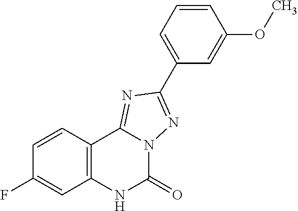 [SMILES: COc1cccc(-c2nc3c4ccc(F)cc4[nH]c(=O)n3n2)c1] |
| | 8-fluoro-2-(3-methoxyphenyl)[1,2,4] |
| | triazolo[1,5-c]quinazolin-5(6H)-one |
| | LC-MS (Method 2): Rt = 0.65 min; |
| | MS (ESIpos): m/z = 311 [M + H]⁺ |
| | ¹H-NMR (400 MHz, DMSO-d₆): δ |
| | [ppm] = 3.87 (s, 3H), 7.12 (ddd, 1H), 7.19 |
| | (dd, 1H), 7.29 (td, 1H), 7.49 (t, 1H), |
| | 7.72 (dd, 1H), 7.81 (dt, 1H), 8.29 (dd, |
| | 1H), 12.45 (s, 1H). |
| Intermediate 110 | 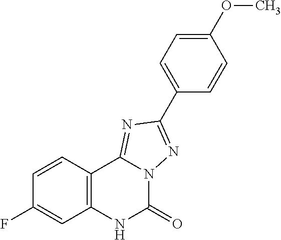 [SMILES: COc1ccc(-c2nc3c4ccc(F)cc4[nH]c(=O)n3n2)cc1] |
| | 8-fluoro-2-(4-methoxyphenyl)[1,2,4] |
| | triazolo[1,5-c]quinazolin-5(6H)-one |
| | LC-MS (Method 2): Rt = 0.65 min; |
| | MS (ESIpos): m/z = 311 [M + H]⁺ |
| | ¹H-NMR (400 MHz, DMSO-d₆): δ |
| | [ppm] = 3.85 (s, 3H), 7.09-7.14 (m, 2H), |
| | 7.19 (dd, 1H), 7.28 (td, 1H), 8.13-8.18 |
| | (m, 2H), 8.28 (dd, 1H), 12.40 (s, 1H). |
| Intermediate 111 | 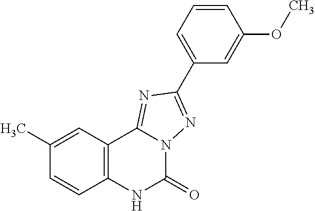 |
| | 2-(3-methoxyphenyl)-9-methyl[1,2,4] |
| | triazolo[1,5-c]quinazolin-5(6H)-one |
| | LC-MS (Method 2): Rₜ = 0.78 min; |
| | MS (ESIneg): m/z = 305 [M − H]⁻ |
| | ¹H-NMR (400 MHz, DMSO-d₆): δ |
| | [ppm] = 2.45 (s, 3H), 3.88 (s, 3H), 7.12 |
| | (ddd, 1H), 7.35 (d, 1H), 7.49 (t, 1H), |
| | 7.54 (dd, 1H), 7.72 (dd, 1H), 7.81 (dt, |
| | 1H), 8.04 (d, 1H), 12.27 (br s, 1H). |
| Intermediate 112 | 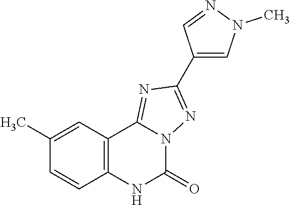 |
| | 9-methyl-2-(1-methyl-1H-pyrazol-4-yl) |
| | [1,2,4]triazolo[1,5-c]quinazolin-5(6H)-one |
| | LC-MS (Method 2): Rₜ = 0.59 min; |
| | MS (ESIpos): m/z = 281 [M + H]⁺ |
| | ¹H-NMR (400 MHz, DMSO-d₆): δ |
| | [ppm] = 2.43 (s, 3H), 3.94 (s, 3H), 7.34 (d, |
| | 1H), 7.52 (dd, 1H), 7.97 (s, 1H), 8.00 |
| | (s, 1H), 8.40 (s, 1H), 12.18 (s 1H). |
| Intermediate 113 | 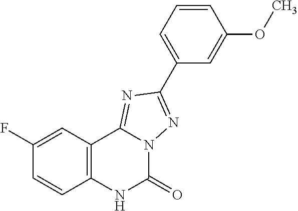 [SMILES: COc1cccc(-c2nc3c4cc(F)ccc4[nH]c(=O)n3n2)c1] |
| | 9-fluoro-2-(3-methoxyphenyl)[1,2,4] |
| | triazolo[1,5-c]quinazolin-5(6H)-one |
| | LC-MS (Method 2): Rt = 0.68 min; |
| | MS (ESIpos): m/z = 311 [M + H]⁺ |
| | ¹H-NMR (400 MHz, DMSO-d₆): δ |
| | [ppm] = 3.88 (s, 3H), 7.13 (ddd, 1H), 7.46- |
| | 7.53 (m, 2H), 7.63 (td, 1H), 7.73 (dd, 1H), |
| | 7.82 (dt, 1H), 7.99 (dd, 1H), 12.41 (br s, 1H). |
| Intermediate 114 | 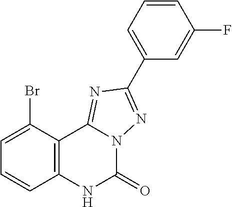 [SMILES: O=c1[nH]c2cccc(Br)c2c2nc(-c3cccc(F)c3)nn12] |
| | 10-bromo-2-(3-fluorophenyl)[1,2,4] |
| | triazolo[1,5-c]quinazolin-5(6H)-one |
| | LC-MS (Method 2): Rₜ = 0.88 min; |
| | MS (ESIpos): m/z = 359 |
| | ¹H-NMR (400 MHz, DMSO-d₆): δ |
| | [ppm] = 7.37-7.44 (m, 1H), 7.46 (dd, 1H), |
| | 7.58 (t, 1H), 7.65 (td, 1H), 7.70 (dd, 1H), |
| | 7.92-7.97 (m, 1H), 8.10 (dt, 1H), 12.54-12.62 (s 1H). |
| Intermediate 115 | 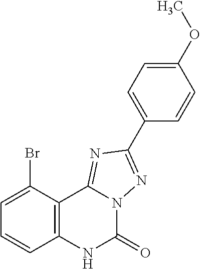 [SMILES: COc1ccc(-c2nc3c4c(Br)cccc4[nH]c(=O)n3n2)cc1] |
| | 10-bromo-2-(4-methoxyphenyl)[1,2,4] |
| | triazolo[1,5-c]quinazolin-5(6H)-one |
| | LC-MS (Method 2): Rₜ = 0.74 min; |
| | MS (ESIpos): m/z = 371 [M + H]⁺ |
| | ¹H-NMR (400 MHz, DMSO-d₆): δ |
| | [ppm] = 3.85 (s, 3H), 7.11-7.16 (m, 2H), |
| | 7.45 (dd, 1H), 7.56 (t, 1H), 7.68 (dd, 1H), |
| | 8.15-8.21 (m, 2H), 12.45 (br s, 1H). |
| Intermediate 116 | 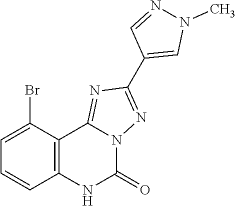 [SMILES: Cn1cc(-c2nc3c4c(Br)cccc4[nH]c(=O)n3n2)cn1] |
| | 10-bromo-2-(1-methyl-1H-pyrazol-4-yl) |
| | [1,2,4]triazolo[1,5-c]quinazolin-5(6H)-one |
| | LC-MS (Method 2): Rₜ = 0.55 min; |
| | MS (ESIneg): m/z = 343 [M − H]⁻ |
| | ¹H-NMR (400 MHz, DMSO-d₆): δ |
| | [ppm] = 3.95 (s, 3H), 7.44 (dd, 1H), 7.55 (t, |
| | 1H), 7.67 (dd, 1H), 8.00 (d, 1H), |
| | 8.39 (s, 1H), 12.42 (br s, 1H). |
| Intermediate 117 | 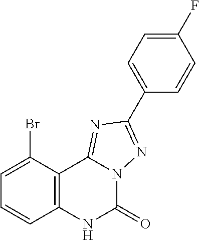 [SMILES: O=c1[nH]c2cccc(Br)c2c2nc(-c3ccc(F)cc3)nn12] |
| | 10-bromo-2-(4-fluorophenyl)[1,2,4] |
| | triazolo[1,5-c]quinazolin-5(6H)-one |
| | LC-MS (Method 2): Rₜ = 0.75 min; |
| | MS (ESIneg): m/z = 357 [M − H]⁻ |
| | ¹H-NMR (400 MHz, DMSO-d₆): δ |
| | [ppm] = 7.39-7.48 (m, 3H), 7.57 (t, 1H), |
| | 7.69 (dd, 1H), 8.25-8.32 (m, 2H), |
| | 12.51 (br s, 1H). |
| Intermediate 118 | 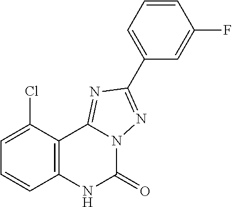 [SMILES: O=c1[nH]c2cccc(Cl)c2c2nc(-c3cccc(F)c3)nn12] |
| | 10-chloro-2-(4-methoxyphenyl)[1,2,4] |
| | triazolo[1,5-c]quinazolin-5(6H)-one |
| | LC-MS (Method 2): Rₜ = 0.73 min; |
| | MS (ESIpos): m/z = 327 [M + H]⁺ |
| | ¹H-NMR (400 MHz, DMSO-d₆): δ |
| | [ppm] = 3.85 (s, 3H), 7.10-7.17 (m, 2H), |
| | 7.41 (dd, 1H), 7.49 (dd, 1H), 7.62-7.68 |
| | (m, 1H), 8.15-8.20 (m, 2H), 12.47 (br s, 1H). |
| Intermediate 119 | 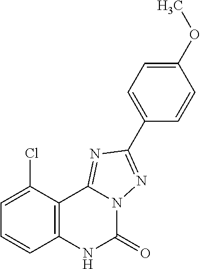 [SMILES: COc1ccc(-c2nc3c4c(Cl)cccc4[nH]c(=O)n3n2)cc1] |
| | 10-chloro-2-(4-methoxyphenyl)[1,2,4] |
| | triazolo[1,5-c]quinazolin-5(6H)-one |
| | LC-MS (Method 2): Rₜ = 0.73 min; |
| | MS (ESIpos): m/z = 327 [M + H]⁺ |
| | ¹H-NMR (400 MHz, DMSO-d₆): δ |
| | [ppm] = 3.85 (s, 3H), 7.10-7.17 (m, 2H), |
| | 7.41 (dd, 1H), 7.49 (dd, 1H), 7.62-7.68 (m, 1H), |
| | 8.15-8.20 (m, 2H), 12.47 (br s, 1H). |
| Intermediate 120 | 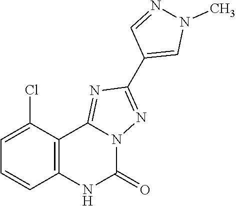 [SMILES: Cn1cc(-c2nc3c4c(Cl)cccc4[nH]c(=O)n3n2)cn1] |
| | 10-chloro-2-(4-fluorophenyl)[1,2,4] |
| | triazolo[1,5-c]quinazolin-5(6H)-one |
| | LC-MS (Method 2): Rₜ = 0.74 min; |
| | MS (ESIpos): m/z = 315 [M + H]⁺ |
| | ¹H-NMR (400 MHz, DMSO-d₆): δ |
| | [ppm] = 7.38-7.46 (m, 3H), 7.50 (dd, 1H), |
| | 7.62-7.69 (m, 1H), 8.24-8.32 (m, 2H), |
| | 12.52 (s, 1H). |
| Intermediate 121 | 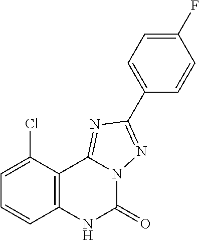 [SMILES: O=c1[nH]c2cccc(Cl)c2c2nc(-c3ccc(F)cc3)nn12] |
| | 10-chloro-2-(4-fluorophenyl)[1,2,4] |
| | triazolo[1,5-c]quinazolin-5(6H)-one |
| | LC-MS (Method 2): Rₜ = 0.74 min; |
| | MS (ESIpos): m/z = 315 [M + H]⁺ |
| | ¹H-NMR (400 MHz, DMSO-d₆): δ |
| | [ppm] = 7.38-7.46 (m, 3H), 7.50 (dd, 1H), |
| | 7.62-7.69 (m, 1H), 8.24-8.32 (m, 2H), |
| | 12.52 (s, 1H). |
| Intermediate 122 | 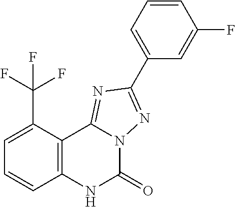 [SMILES: O=c1[nH]c2cccc(C(F)(F)F)c2c2nc(-c3cccc(F)c3)nn12] |
| | 2-(3-fluorophenyl)-10-(trifluoromethyl) |
| | [1,2,4]triazolo[1,5-c]quinazolin- |
| | 5(6H)-one |
| | LC-MS (Method 2): Rₜ = 0.79 min; |
| | MS (ESIpos): m/z = 349 [M + H]⁺ |
| | ¹H-NMR (400 MHz, DMSO-d₆): δ |
| | [ppm] = 7.38-7.46 (m, 1H), 7.66 (td, 1H), |
| | 7.76 (dd, 1H), 7.81-7.93 (m, 3H), |
| | 8.07 (dt, 1H), 12.71 (br s, 1H). |
| Intermediate 123 | 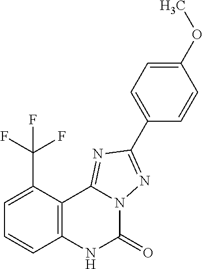 [SMILES: COc1ccc(-c2nc3c4c(C(F)(F)F)cccc4[nH]c(=O)n3n2)cc1] |
| | 2-(4-methoxyphenyl)-10- |
| | (trifluoromethyl)[1,2,4]triazolo[1,5- |
| | c]quinazolin-5(6H)-one |
| | LC-MS (Method 2): Rₜ = 0.77 min; |
| | MS (ESIpos): m/z = 361 [M + H]⁺ |
| | ¹H-NMR (400 MHz, DMSO-d₆): δ |
| | [ppm] = 3.85 (s, 3H), 7.12-7.17 (m, 2H), |
| | 7.75 (dd, 1H), 7.80-7.89 (m, 2H), |
| | 8.13-8.18 (m, 2H), 12.62 (br s, 1H). |
| Intermediate 124 | 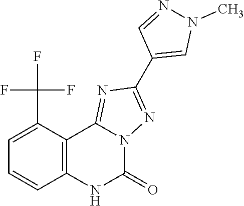 [SMILES: Cn1cc(-c2nc3c4c(C(F)(F)F)cccc4[nH]c(=O)n3n2)cn1] |
| | 2-(1-methyl-1H-pyrazol-4-yl)-10- |
| | (trifluoromethyl)[1,2,4]triazolo[1,5- |
| | c]quinazolin-5(6H)-one |
| | LC-MS (Method 2): Rₜ = 0.62 min; |
| | MS (ESIpos): m/z = 335 [M + H]⁺ |
| | ¹H-NMR (400 MHz, DMSO-d₆): δ |
| | [ppm] = 3.95 (s, 3H), 7.74 (dd, 1H), 7.79- |
| | 7.88 (m, 2H), 7.98 (d, 1H), |
| | 8.36 (s, 1H), 12.58 (br s, 1H). |
| Intermediate 125 | 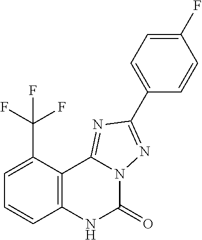 [SMILES: O=c1[nH]c2cccc(C(F)(F)F)c2c2nc(-c3ccc(F)cc3)nn12] |
| | 2-(4-fluorophenyl)-10-(trifluoromethyl) |
| | [1,2,4]triazolo[1,5-c]quinazolin- |
| | 5(6H)-one |
| | LC-MS (Method 2): Rₜ = 0.79 min; |
| | MS (ESIpos): m/z = 349 [M + H]⁺ |
| | ¹H-NMR (400 MHz, DMSO-d₆): δ |
| | [ppm] = 7.40-7.48 (m, 2H), 7.75 (dd, 1H), |
| | 7.81-7.90 (m, 2H), 8.22-8.29 |
| | (m, 2H), 12.68 (br s, 1H). |
| Intermediate 126 | 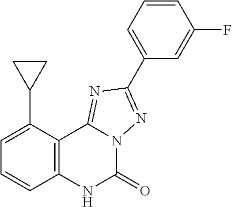 [SMILES: O=c1[nH]c2cccc(C3CC3)c2c2nc(-c3cccc(F)c3)nn12] |
| | 10-cyclopropyl-2-(3-fluorophenyl)[1,2,4] |
| | triazolo[1,5-c]quinazolin-5(6H)-one |
| | LC-MS (Method 2): Rₜ = 0.93 min; |
| | MS (ESIpos): m/z = 321 [M + H]⁺ |
| Intermediate 127 | 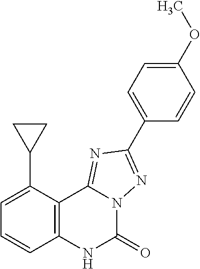 [SMILES: COc1ccc(-c2nc3c4c(C5CC5)cccc4[nH]c(=O)n3n2)cc1] |
| | 10-cyclopropyl-2-(4-methoxyphenyl) |
| | [1,2,4]triazolo[1,5-c]quinazolin- |
| | 5(6H)-one |
| | LC-MS (Method 2): Rₜ = 0.95 min; |
| | MS (ESIpos): m/z = 333 [M + H]⁺ |
| Intermediate 128 | 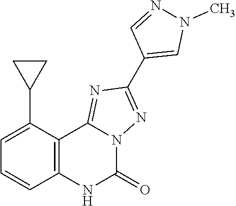 [SMILES: Cn1cc(-c2nc3c4c(C5CC5)cccc4[nH]c(=O)n3n2)cn1] |
| | 10-cyclopropyl-2-(1-methyl-1H- |
| | pyrazol-4-yl)[1,2,4]triazolo[1,5- |
| | c]quinazolin-5(6H)-one |
| | LC-MS (Method 2): Rₜ = 0.69 min; |
| | MS (ESIpos): m/z = 307 [M + H]⁺ |
| | ¹H-NMR (400 MHz, DMSO-d₆): δ |
| | [ppm] = 0.85 (br d, 2H), 1.19 (br d, 2H), |
| | 3.73-3.84 (m, 1H), 3.94 (s, 3H), 6.90 |
| | (br d, 1H), 7.23 (br d, 1H), 7.54 (br t, |
| | 1H), 8.01 (s, 1H), 8.41 (s, 1H), 12.19 (brs, 1H). |
| Intermediate 129 | 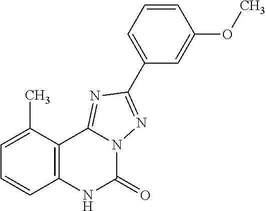 [SMILES: COc1cccc(-c2nc3c4c(C)cccc4[nH]c(=O)n3n2)c1] |
| | 2-(3-methoxyphenyl)-10-methyl[1,2,4] |
| | triazolo[1,5-c]quinazolin-5(6H)-one |
| | LC-MS (Method 2): Rₜ = 0.92 min; |
| | MS (ESIpos): m/z = 307 [M + H]⁺ |
| | ¹H-NMR (400 MHz, DMSO-d₆): δ |
| | [ppm] = 2.97 (s, 3H), 3.87 (s, 3H), 7.12 |
| | (ddd, 1H), 7.22-7.27 (m, 1H), 7.30 (d, 1H), |
| | 7.50 (t, 1H), 7.54-7.60 (m, 1H), |
| | 7.73 (dd, 1H), 7.83 (dt, 1H), 12.29 (br s, 1H). |
| Intermediate 130 | 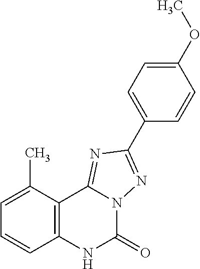 [SMILES: COc1ccc(-c2nc3c4c(C)cccc4[nH]c(=O)n3n2)cc1] |
| | 2-(4-methoxyphenyl)-10-methyl[1,2,4] |
| | triazolo[1,5-c]quinazolin-5(6H)-one |
| | LC-MS (Method 2): Rₜ = 0.89 min; |
| | MS (ESIpos): m/z = 307 [M + H]⁺ |
| | ¹H-NMR (400 MHz, DMSO-d₆): δ |
| | [ppm] = 2.96 (s, 3H), 3.85 (s, 3H), 7.10- |
| | 7.15 (m, 2H), 7.24 (d, 1H), 7.29 (d, 1H), |
| | 7.53-7.60 (m, 1H), 8.14-8.19 (m, |
| | 2H), 12.24 (s, 1H). |
| Intermediate 131 | 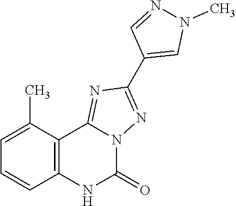 [SMILES: Cc1cccc2[nH]c(=O)n3nc(-c4cnn(C)c4)nc3c12] |
| | 10-methyl-2-(1-methyl-1H-pyrazol-4-yl) |
| | [1,2,4]triazolo[1,5-c]quinazolin- |
| | 5(6H)-one |
| | LC-MS (Method 2): Rₜ = 0.68 min; |
| | MS (ESIpos): m/z = 281 [M + H]⁺ |
| | ¹H-NMR (400 MHz, DMSO-d₆): δ |
| | [ppm] = 2.93 (s, 3H), 3.94 (s, 3H), 7.22 (d, |
| | 1H), 7.29 (d, 1H), 7.51-7.58 (m, 1H), |
| | 8.01 (s, 1H), 8.41 (s, 1H), 12.20 (brs, 1H). |
| Intermediate 132 | 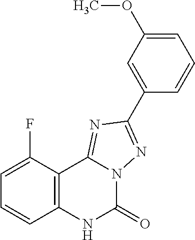 [SMILES: COc1cccc(-c2nc3c4c(F)cccc4[nH]c(=O)n3n2)c1] |
| | 10-fluoro-2-(3-methoxyphenyl)[1,2,4] |
| | triazolo[1,5-c]quinazolin-5(6H)-one |
| | LC-MS (Method 2): Rt = 0.61 min; |
| | MS (ESIpos): m/z = 311 [M + H]⁺ |
| | ¹H-NMR (400 MHz, DMSO-d₆): δ |
| | [ppm] = 3.87 (s, 3H), 7.13 (dd, 1H), 7.23- |
| | 7.32 (m, 2H), 7.50 (t, 1H), 7.66-7.77 (m, 2H), |
| | 7.82 (br d, 1H), 12.53 (s, 1H). |

### Intermediate 133

### 5-chloro-7-fluoro-2-(3-fluorophenyl)[1,2,4]triazolo[1,5-c]quinazoline

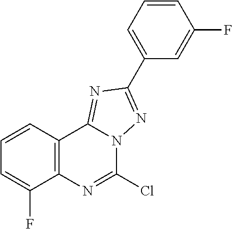 [SMILES: Fc1cccc(-c2nc3c4cccc(F)c4nc(Cl)n3n2)c1]

7-Fluoro-2-(3-fluorophenyl)[1,2,4]triazolo[1,5-c]quinazolin-5(6H)-one (100 mg, 335 μmol) was solubilised in phosphorus(V) oxychloride (1.1 mL, 12 mmol), N,N-diisopropylethylamine (580 μL, 3.4 mmol) was added carefully and the mixture was stirred overnight at 110° C. The reaction mixture was cooled to rt, poured into ice and stirred for 1 h. The solid was filtered, washed with water and dried at 60° C. under reduced pressure to give 91.0 mg (97% purity, 83% yield) of the title compound. The compound was used without further purification.

LC-MS (method 2): Rₜ=1.40 min; MS (ESIpos): m/z=317 [M+H]⁺

¹H-NMR (400 MHz, DMSO-d6): δ [ppm]=7.43-7.50 (m, 1H), 7.68 (td, 1H), 7.84-7.92 (m, 2H), 8.00 (ddd, 1H), 8.15 (dt, 1H), 8.31-8.37 (m, 1H).

The following intermediates were prepared similarly:

| | Structure |
|---|---|
| | IUPAC-Name |
| | LC-MS (method): Retention time; Mass found |
| Intermediate | ¹H-NMR |
| Intermediate 134 | 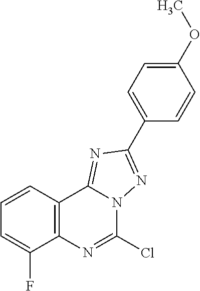 [SMILES: COc1ccc(-c2nc3c4cccc(F)c4nc(Cl)n3n2)cc1] |
| | 5-chloro-7-fluoro-2-(4-methoxyphenyl)[1,2,4]triazolo[1,5- |
| | c]quinazoline |
| | LC-MS (Method 2): Rt = 1.35 min; MS (ESIpos): m/z = 329 [M + H]⁺ |
| | ¹H-NMR (400 MHz, DMSO-d₆): δ [ppm] = 3.87 (s, 3H), 7.11-7.18 (m, 2H), |
| | 7.81-7.89 (m, 2H), 8.21-8.27 (m, 2H), 8.29-8.34 (m, 1H). |
| Intermediate 135 | 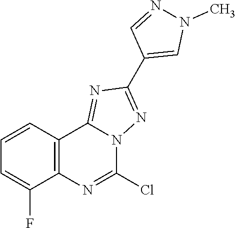 [SMILES: Cn1cc(-c2nc3c4cccc(F)c4nc(Cl)n3n2)cn1] |
| | 5-chloro-7-fluoro-2-(1-methyl-1H-pyrazol-4-yl)[1,2,4]triazolo[1,5- |
| | c]quinazoline |
| | LC-MS (Method 2): Rₜ = 0.98 min; MS (ESIpos): m/z = 303 [M + H]⁺ |
| | ¹H-NMR (400 MHz, DMSO-d₆): δ [ppm] = 3.96 (s, 3H), 7.80-7.87 (m, 2H), |
| | 8.10 (s, 1H), 8.23-8.28 (m, 1H), 8.55 (s, 1H). |
| Intermediate 136 | 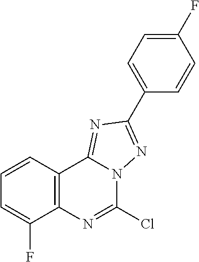 [SMILES: Fc1ccc(-c2nc3c4cccc(F)c4nc(Cl)n3n2)cc1] |
| | 5-chloro-7-fluoro-2-(4-fluorophenyl)[1,2,4]triazolo[1,5-c]quinazoline |
| | LC-MS (Method 2): Rₜ = 1.38 min; MS (ESIpos): m/z = 317 [M + H]⁺ |
| | ¹H-NMR (400 MHz, DMSO-d₆): δ [ppm] = 7.41-7.48 (m, 2H), 7.83-7.90 |
| | (m, 2H), 8.30-8.38 (m, 3H). |
| Intermediate 137 | 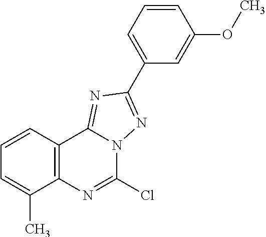 [SMILES: COc1cccc(-c2nc3c4cccc(C)c4nc(Cl)n3n2)c1] |
| | 5-chloro-2-(3-methoxyphenyl)-7-methyl[1,2,4]triazolo[1,5- |
| | c]quinazoline |
| | LC-MS (Method 2): Rₜ = 1.53 min; MS (ESIpos): m/z = 325 [M + H]⁺ |
| | ¹H-NMR (400 MHz, DMSO-d₆): δ [ppm] = 2.70 (s, 3H), 3.89 (s, 3H), 7.16 |
| | (ddd, 1H), 7.52 (t, 1H), 7.71-7.79 (m, 2H), 7.85 (d, 1H), 7.89 (d, 1H), |
| | 8.36 (d, 1H). |
| Intermediate 138 | 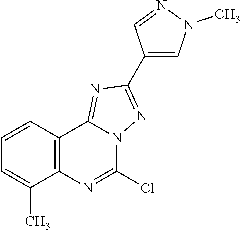 [SMILES: Cc1cccc2c1nc(Cl)n1nc(-c3cnn(C)c3)nc21] |
| | 5-chloro-7-methyl-2-(1-methyl-1H-pyrazol-4-yl)[1,2,4]triazolo[1,5- |
| | c]quinazoline |
| | LC-MS (Method 2): Rₜ = 1.13 min; MS (ESIpos): m/z = 299 [M + H]⁺ |
| | ¹H-NMR (400 MHz, DMSO-d₆): δ [ppm] = 2.68 (s, 3H), 3.96 (s, 3H), 7.69- |
| | 7.75 (m, 1H), 7.80-7.86 (m, 1H), 8.08 (s, 1H), 8.28 (dd, 1H), 8.53 (s, 1H). |
| Intermediate 139 | 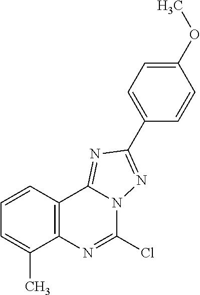 [SMILES: COc1ccc(-c2nc3c4cccc(C)c4nc(Cl)n3n2)cc1] |
| | 5-chloro-2-(4-methoxyphenyl)-7-methyl[1,2,4]triazolo[1,5- |
| | c]quinazoline |
| | LC-MS (Method 2): Rₜ = 1.52 min; MS (ESIpos): m/z = 325 [M + H]⁺ |
| | ¹H-NMR (400 MHz, DMSO-d₆): δ [ppm] = 2.69 (s, 3H), 3.86 (s, 3H), 7.11- |
| | 7.17 (m, 2H), 7.70-7.76 (m, 1H), 7.83 (d, 1H), 8.19-8.25 (m, 2H), 8.30- |
| | 8.36 (m, 1H). |
| Intermediate 140 | 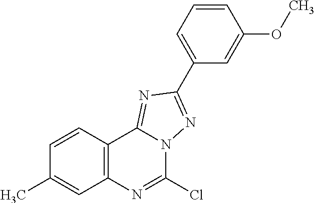 |
| | 5-chloro-2-(3-methoxyphenyl)-8-methyl[1,2,4]triazolo[1,5- |
| | c]quinazoline |
| | LC-MS (Method 2): Rₜ = 1.44 min; MS (ESIpos): m/z = 325 [M + H]⁺ |
| | ¹H-NMR (400 MHz, DMSO-d₆): δ [ppm] = 2.58 (s, 3H), 3.89 (s, 3H), 7.16 |
| | (ddd, 1H), 7.52 (t, 1H), 7.70 (dd, 1H), 7.77 (dd, 1H), 7.85 (s, 1H), 7.88 (dt, |
| | 1H), 8.41 (d, 1H). |
| Intermediate 141 | 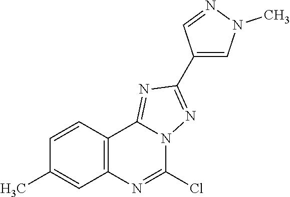 |
| | 5-chloro-8-methyl-2-(1-methyl-1H-pyrazol-4-yl)[1,2,4]triazolo[1,5- |
| | c]quinazoline |
| | LC-MS (Method 2): Rₜ = 1.07 min; MS (ESIpos): m/z = 299 [M + H]⁺ |
| | ¹H-NMR (400 MHz, DMSO-d₆): δ [ppm] = 2.56 (s, 3H), 3.95 (s, 4H), 7.67 |
| | (dd, 1H), 7.82 (s, 1H), 8.08 (d, 1H), 8.33 (d, 1H), 8.52 (s, 1H). |
| Intermediate 142 | 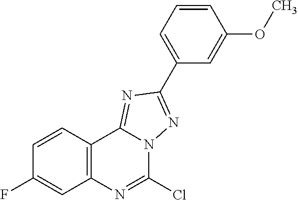 [SMILES: COc1cccc(-c2nc3c4ccc(F)cc4nc(Cl)n3n2)c1] |
| | 5-chloro-8-fluoro-2-(3-methoxyphenyl)[1,2,4]triazolo[1,5- |
| | c]quinazoline |
| | LC-MS (Method 2): Rt = 1.40 min; MS (ESIpos): m/z = 329 [M + H]⁺ |
| | ¹H-NMR (400 MHz, DMSO-d₆): δ [ppm] = 3.89 (s, 3H), 7.17 (ddd, 1H), 7.53 |
| | (t, 1H), 7.73-7.80 (m, 2H), 7.88 (dt, 1H), 7.93 (dd, 1H), 8.59 (dd, 1H). |
| Intermediate 143 | 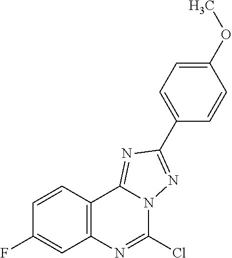 [SMILES: COc1ccc(-c2nc3c4ccc(F)cc4nc(Cl)n3n2)cc1] |
| | 5-chloro-8-fluoro-2-(4-methoxyphenyl)[1,2,4]triazolo[1,5- |
| | c]quinazoline |
| | LC-MS (Method 2): Rt = 1.39 min; MS (ESIpos): m/z = 329 [M + H]⁺ |
| | ¹H-NMR (400 MHz, DMSO-d₆): δ [ppm] = 3.86 (s, 3H), 7.14-7.17 (m, 2H), |
| | 7.75 (td, 1H), 7.91 (dd, 1H), 8.20-8.24 (m, 2H), 8.57 (dd, 1H). |
| Intermediate 144 | 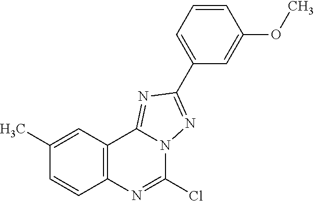 |
| | 5-chloro-2-(3-methoxyphenyl)-9-methyl[1,2,4]triazolo[1,5- |
| | c]quinazoline |
| | LC-MS (Method 2): Rₜ = 1.48 min; MS (ESIpos): m/z = 325 [M + H]⁺ |
| | ¹H-NMR (400 MHz, DMSO-d₆): δ [ppm] = 2.60 (s, 3H), 3.89 (s, 3H), 7.16 |
| | (ddd, 1H), 7.52 (t, 1H), 7.76-7.83 (m, 2H), 7.88 (dt, 1H), 7.93 (d, 1H), |
| | 8.33 (dd, 1H). |
| Intermediate 145 | 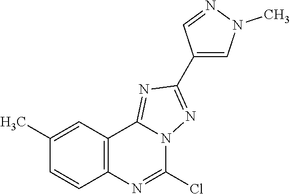 |
| | 5-chloro-9-methyl-2-(1-methyl-1H-pyrazol-4-yl)[1,2,4]triazolo[1,5- |
| | c]quinazoline |
| | LC-MS (Method 2): Rt = 1.09 min; MS (ESIpos): m/z = 299 [M + H]⁺ |
| Intermediate 146 | 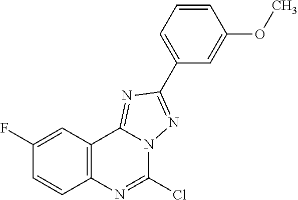 [SMILES: COc1cccc(-c2nc3c4cc(F)ccc4nc(Cl)n3n2)c1] |
| | 5-chloro-9-fluoro-2-(3-methoxyphenyl)[1,2,4]triazolo[1,5- |
| | c]quinazoline |
| | LC-MS (Method 2): Rt = 1.41 min; MS (ESIpos): m/z = 329 [M + H]⁺ |
| | ¹H-NMR (400 MHz, DMSO-d₆): δ [ppm] = 3.89 (s, 3H), 7.17 (ddd, 1H), 7.53 |
| | (t, 1H), 7.78 (dd, 1H), 7.84-7.91 (m, 2H), 8.13 (dd, 1H), 8.26 (dd, 1H). |
| Intermediate 147 | 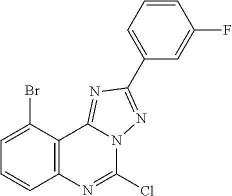 [SMILES: Fc1cccc(-c2nc3c4c(Br)cccc4nc(Cl)n3n2)c1] |
| | 10-bromo-5-chloro-2-(3-fluorophenyl)[1,2,4]triazolo[1,5-c]quinazoline |
| | LC-MS (Method 2): Rₜ = 1.54 min; MS (ESIpos): m/z = 377 [M + H]⁺ |
| Intermediate 148 | 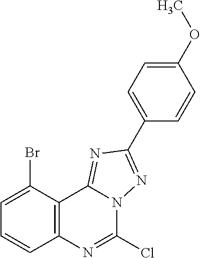 [SMILES: COc1ccc(-c2nc3c4c(Br)cccc4nc(Cl)n3n2)cc1] |
| | 10-bromo-5-chloro-2-(4-methoxyphenyl)[1,2,4]triazolo[1,5- |
| | c]quinazoline |
| | LC-MS (Method 2): Rₜ = 1.49 min; MS (ESIpos): m/z = 389 [M + H]⁺ |
| Intermediate 149 | 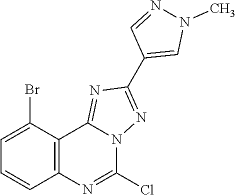 [SMILES: Cn1cc(-c2nc3c4c(Br)cccc4nc(Cl)n3n2)cn1] |
| | 10-bromo-5-chloro-2-(1-methyl-1H-pyrazol-4-yl)[1,2,4]triazolo[1,5- |
| | c]quinazoline |
| | LC-MS (Method 2): Rₜ = 1.12 min; MS (ESIpos): m/z = 363 [M + H]⁺ |
| Intermediate 150 | 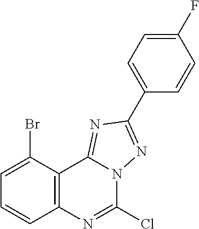 [SMILES: Fc1ccc(-c2nc3c4c(Br)cccc4nc(Cl)n3n2)cc1] |
| | 10-bromo-5-chloro-2-(4-fluorophenyl)[1,2,4]triazolo[1,5-c]quinazoline |
| | LC-MS (Method 2): Rₜ = 1.53 min; MS (ESIpos): m/z = 377 [M + H]⁺ |
| Intermediate 151 | 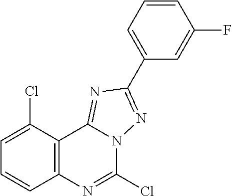 [SMILES: Fc1cccc(-c2nc3c4c(Cl)cccc4nc(Cl)n3n2)c1] |
| | 5,10-dichloro-2-(3-fluorophenyl)[1,2,4]triazolo[1,5-c]quinazoline |
| | LC-MS (Method 2): Rₜ = 1.51 min; MS (ESIpos): m/z = 333 [M + H]⁺ |
| Intermediate 152 | 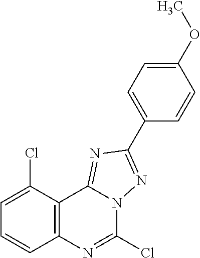 [SMILES: COc1ccc(-c2nc3c4c(Cl)cccc4nc(Cl)n3n2)cc1] |
| | 5,10-dichloro-2-(4-methoxyphenyl)[1,2,4]triazolo[1,5-c]quinazoline |
| | LC-MS (Method 2): Rₜ = 1.46 min; MS (ESIpos): m/z = 345 [M + H]⁺ |
| | ¹H-NMR (400 MHz, DMSO-d₆): δ [ppm] = 3.87 (s, 3H), 7.14-7.19 (m, 2H), |
| | 7.89-8.02 (m, 3H), 8.22-8.28 (m, 2H). |
| Intermediate 153 | 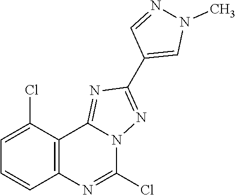 [SMILES: Cn1cc(-c2nc3c4c(Cl)cccc4nc(Cl)n3n2)cn1] |
| | 5,10-dichloro-2-(1-methyl-1H-pyrazol-4-yl)[1,2,4]triazolo[1,5- |
| | c]quinazoline |
| | LC-MS (Method 2): Rₜ = 1.08 min; MS (ESIpos): m/z = 319 [M + H]⁺ |
| Intermediate 154 | 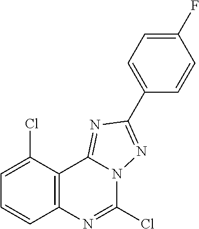 [SMILES: Fc1ccc(-c2nc3c4c(Cl)cccc4nc(Cl)n3n2)cc1] |
| | 5,10-dichloro-2-(4-fluorophenyl)[1,2,4]triazolo[1,5-c]quinazoline |
| | LC-MS (Method 2): Rₜ = 1.50 min; MS (ESIpos): m/z = 333 [M + H]⁺ |
| Intermediate 155 | 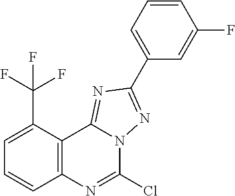 [SMILES: Fc1cccc(-c2nc3c4c(C(F)(F)F)cccc4nc(Cl)n3n2)c1] |
| | 5-chloro-2-(3-fluorophenyl)-10-(trifluoromethyl)[1,2,4]triazolo[1,5- |
| | c]quinazoline |
| | LC-MS (Method 2): Rₜ = 1.52 min; MS (ESIpos): m/z = 367 [M + H]⁺ |
| Intermediate 156 | 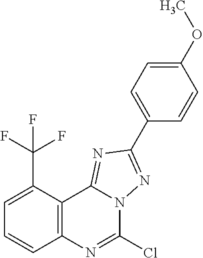 [SMILES: COc1ccc(-c2nc3c4c(C(F)(F)F)cccc4nc(Cl)n3n2)cc1] |
| | 5-chloro-2-(4-methoxyphenyl)-10-(trifluoromethyl)[1,2,4]triazolo[1,5- |
| | c]quinazoline |
| | LC-MS (Method 2): Rₜ = 1.49 min; MS (ESIpos): m/z = 379 [M + H]⁺ |
| Intermediate 157 | 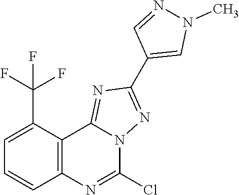 [SMILES: Cn1cc(-c2nc3c4c(C(F)(F)F)cccc4nc(Cl)n3n2)cn1] |
| | 5-chloro-2-(1-methyl-1H-pyrazol-4-yl)-10- |
| | (trifluoromethyl)[1,2,4]triazolo[1,5-c]quinazoline |
| | LC-MS (Method 2): Rₜ = 1.17 min; MS (ESIpos): m/z = 353 [M + H]⁺ |
| Intermediate 158 | 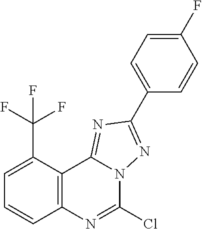 [SMILES: Fc1ccc(-c2nc3c4c(C(F)(F)F)cccc4nc(Cl)n3n2)cc1] |
| | 5-chloro-2-(4-fluorophenyl)-10-(trifluoromethyl)[1,2,4]triazolo[1,5- |
| | c]quinazoline |
| | LC-MS (Method 2): Rₜ = 1.52 min; MS (ESIpos): m/z = 367 [M + H]⁺ |
| Intermediate 159 | 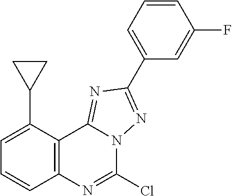 [SMILES: Fc1cccc(-c2nc3c4c(C5CC5)cccc4nc(Cl)n3n2)c1] |
| | 5-chloro-10-cyclopropyl-2-(3-fluorophenyl)[1,2,4]triazolo[1,5- |
| | c]quinazoline |
| | LC-MS (Method 2): Rₜ = 1.63 min; MS (ESIpos): m/z = 339 [M + H]⁺ |
| Intermediate 160 | 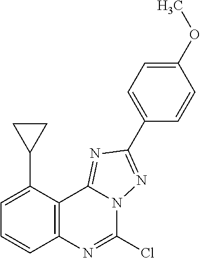 [SMILES: COc1ccc(-c2nc3c4c(C5CC5)cccc4nc(Cl)n3n2)cc1] |
| | 5-chloro-10-cyclopropyl-2-(4-methoxyphenyl)[1,2,4]triazolo[1,5- |
| | c]quinazoline |
| | LC-MS (Method 2): Rₜ = 1.58 min; MS (ESIpos): m/z = 351 [M + H]⁺ |
| Intermediate 161 | 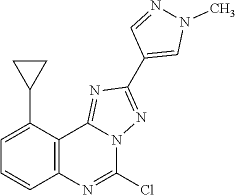 [SMILES: Cn1cc(-c2nc3c4c(C5CC5)cccc4nc(Cl)n3n2)cn1] |
| | 5-chloro-10-cyclopropyl-2-(1-methyl-1H-pyrazol-4- |
| | yl)[1,2,4]triazolo[1,5-c]quinazoline |
| | LC-MS (Method 2): Rₜ = 1.25 min; MS (ESIpos): m/z = 325 [M + H]⁺ |
| Intermediate 162 | 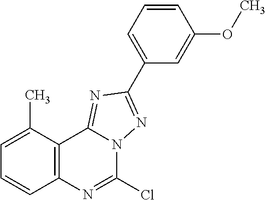 [SMILES: COc1cccc(-c2nc3c4c(C)cccc4nc(Cl)n3n2)c1] |
| | 5-chloro-2-(3-methoxyphenyl)-10-methyl[1,2,4]triazolo[1,5- |
| | c]quinazoline |
| | LC-MS (Method 2): Rₜ = 1.55 min; MS (ESIpos): m/z = 325 [M + H]⁺ |
| | ¹H-NMR (400 MHz, DMSO-d₆): δ [ppm] = 3.09 (s, 3H), 3.88 (s, 3H), 7.15 |
| | (ddd, 1H), 7.52 (t, 1H), 7.65-7.70 (m, 1H), 7.76 (dd, 1H), 7.80-7.86 (m, |
| | 2H), 7.86-7.90 (m, 1H). |
| Intermediate 163 | 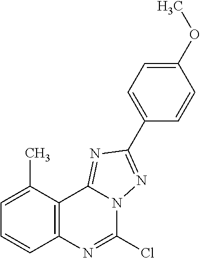 [SMILES: COc1ccc(-c2nc3c4c(C)cccc4nc(Cl)n3n2)cc1] |
| | 5-chloro-2-(4-methoxyphenyl)-10-methyl[1,2,4]triazolo[1,5- |
| | c]quinazoline |
| | LC-MS (Method 2): Rₜ = 1.54 min; MS (ESIpos): m/z = 325 [M + H]⁺ |
| | ¹H-NMR (500 MHz, DMSO-d₆): δ [ppm] = 3.10 (s, 3H), 3.86 (s, 3H), 7.16 |
| | (d, 2H), 7.66-7.70 (m, 1H), 7.81-7.87 (m, 2H), 8.22-8.27 (m, 2H). |
| Intermediate 164 | 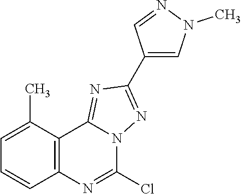 [SMILES: Cc1cccc2nc(Cl)n3nc(-c4cnn(C)c4)nc3c12] |
| | 5-chloro-10-methyl-2-(1-methyl-1H-pyrazol-4-yl)[1,2,4]triazolo[1,5- |
| | c]quinazoline |
| | LC-MS (Method 2): Rₜ = 1.18 min; MS (ESIpos): m/z = 299 [M + H]⁺ |
| | ¹H-NMR (400 MHz, DMSO-d₆): δ [ppm] = 3.06 (s, 3H), 3.96 (s, 3H), 7.64- |
| | 7.68 (m, 1H), 7.80-7.85 (m, 2H), 8.08 (s, 1H), 8.52 (s, 1H). |
| Intermediate 165 | 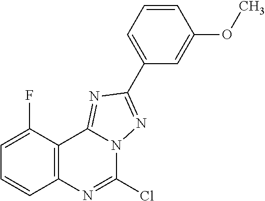 [SMILES: COc1cccc(-c2nc3c4c(F)cccc4nc(Cl)n3n2)c1] |
| | 5-chloro-10-fluoro-2-(3-methoxyphenyl)[1,2,4]triazolo[1,5- |
| | c]quinazoline |
| | LC-MS (Method 2): Rt = 0.92 min; MS (ESIpos): m/z = 329 [M + H]⁺ |
| | ¹H-NMR (400 MHz, DMSO-d₆): δ [ppm] = 3.89 (s, 3H), 7.17 (ddd, 1H), 7.53 |
| | (t, 1H), 7.70-7.79 (m, 2H), 7.86-7.91 (m, 2H), 7.94-8.02 (m, 1H). |

### Intermediate 166

### benzyl 6-{[2-(1-methyl-1H-pyrazol-4-yl)[1,2,4]triazolo[1,5-c]quinazolin-5-yl]amino}-5-oxo-1,4-diazepane-1-carboxylate

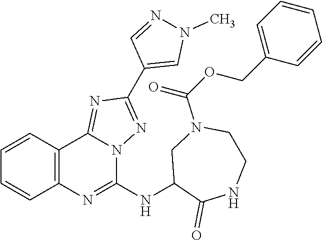 [SMILES: Cn1cc(-c2nc3c4ccccc4nc(NC4CN(C(=O)OCc5ccccc5)CCNC4=O)n3n2)cn1]

5-Chloro-2-(1-methyl-1H-pyrazol-4-yl)[1,2,4]triazolo[1,5-c]quinazoline, (211 mg, 739 μmol)-, benzyl 6-amino-5-oxo-1,4-diazepane-1-carboxylate (292 mg, 1.11 mmol) and N,N-diisopropylethylamine (260 μL, 1.5 mmol) were stirred in DMSO (3.0 mL) for 2 h at 60° C. The mixture was cooled to rt and diluted with water. The solid was filtered, washed with water and dried under reduced pressure at 60° C. to give 182 mg (55% purity, 26% yield) of the title compound that was used without further purification.

LC-MS (method 2): Rₜ=1.08 min; MS (ESIpos): m/z=512 [M+H]⁺

### Intermediate 167

### benzyl 6-{[2-(1-methyl-1H-pyrazol-4-yl)[1,2,4]triazolo[1,5-c]quinazolin-5-yl]amino}-5-oxo-1,4-diazepane-1-carboxylate, Enantiomer 1

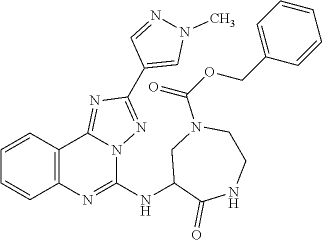 [SMILES: Cn1cc(-c2nc3c4ccccc4nc(NC4CN(C(=O)OCc5ccccc5)CCNC4=O)n3n2)cn1]

Chiral HPLC separation of benzyl 6-{[2-(1-methyl-1H-pyrazol-4-yl)[1,2,4]triazolo[1,5-c]quinazolin-5-yl]amino}-5-oxo-1,4-diazepane-1-carboxylate was performed (Instrument: Labomatic HD5000, Labocord-5000; Gilson GX-241, Labcol Vario 4000; Column: Chiralpak IA 5μ 250×30 mm; Eluent: methanol+0.1 vol-% diethylamine (99%)/ethanol 50:50%; flow rate 40.0 mL/min; UV 254 nm).

Retention time of enantiomer 1: 5.73 min; [α]²⁰_{D}: −96° (c=1) in DMSO.

Instrument: Agilent HPLC 1260; Column: Chiralpak IA 3μ 100×4.6 mm; Eluent: methanol+0.1 vol-% diethylamine (99%)/ethanol 50:50; flow rate 1.4 mL/min; temperature: 25° C.; DAD 254 nm

### Intermediate 168

### benzyl 6-{[2-(1-methyl-1H-pyrazol-4-yl)[1,2,4]triazolo[1,5-c]quinazolin-5-yl]amino}-5-oxo-1,4-diazepane-L-carboxylate, Enantiomer 2

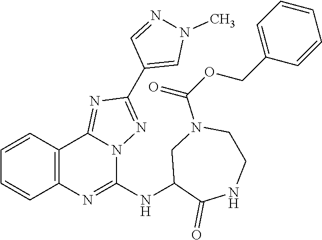 [SMILES: Cn1cc(-c2nc3c4ccccc4nc(NC4CN(C(=O)OCc5ccccc5)CCNC4=O)n3n2)cn1]

The title compound was prepared as described for intermediate 162.

Retention time of enantiomer 2: 9.64 min [α]²⁰_{D}: +952 (c=1) in DMSO

The following intermediates were prepared in analogy to intermediate 161:

| | Structure |
|---|---|
| | IUPAC-Name |
| | LC-MS (method): Retention time; Mass found |
| Intermediate | ¹H-NMR |
| Intermediate 169 | 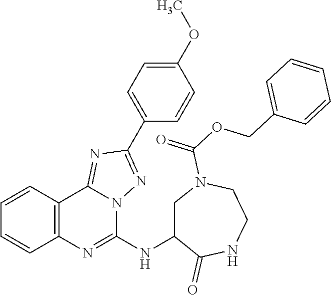 [SMILES: COc1ccc(-c2nc3c4ccccc4nc(NC4CN(C(=O)OCc5ccccc5)CCNC4=O)n3n2)cc1] |
| | benzyl 6-{[2-(4-methoxyphenyl)[1,2,4]triazolo[1,5-c]quinazolin-5- |
| | yl]amino}-5-oxo-1,4-diazepane-1-carboxylate |
| | LC-MS (Method 2): Rₜ = 1.35 min; MS (ESIneg): m/z = 536 [M − H]⁻ |
| | ¹H-NMR (400 MHz, DMSO-d₆): δ [ppm] = 2.90-3.15 (m, 1H), 3.42-3.56 (m, |
| | 1H), 3.86 (s, 3H), 4.10-4.27 (m, 1H), 4.55-4.70 (m, 1H), 4.88-5.01 (m, |
| | 1H), 5.20 (s, 2H), 7.11-7.24 (m, 4H), 7.31-7.49 (m, 5H), 7.60-7.80 (m, |
| | 1H), 7.85 (d, 1H), 8.19-8.26 (m, 2H), 8.30 (br d, 1H), 8.37-8.54 (m, 1H) |
| Intermediate 170 | 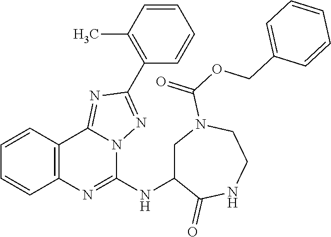 [SMILES: Cc1ccccc1-c1nc2c3ccccc3nc(NC3CN(C(=O)OCc4ccccc4)CCNC3=O)n2n1] |
| | benzyl 6-{[2-(2-methylphenyl)[1,2,4]triazolo[1,5-c]quinazolin-5- |
| | yl]amino}-5-oxo-1,4-diazepane-1-carboxylate |
| | LC-MS (Method 2): Rₜ = 1.45 min; MS (ESIneg): m/z = 520 [M − H]⁻ |
| | ¹H-NMR (400 MHz, DMSO-d₆): δ [ppm] =), 2.73 (s, 3H), 2.92-3.13 (m, 1H), |
| | 3.44-3.55 (m, 1H), 4.13-4.29 (m, 1H), 4.58-4.72 (m, 1H), 4.87-5.00 (m, |
| | 1H), 5.20 (br s, 2H), 7.19 (br s, 2H), 7.29-7.52 (m, 8H), 7.60-7.81 (m, 1H), |
| | 7.89 (d, 1H), 8.14 (br d, 1H), 8.31 (br d, 1H), 8.36-8.53 (m, 1H). |
| Intermediate 171 | 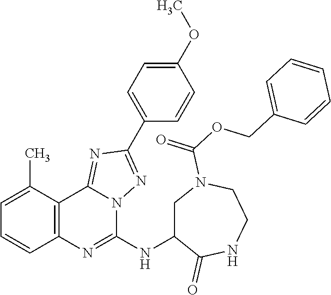 [SMILES: COc1ccc(-c2nc3c4c(C)cccc4nc(NC4CN(C(=O)OCc5ccccc5)CCNC4=O)n3n2)cc1] |
| | benzyl 6-{[2-(4-methoxyphenyl)-10-methyl[1,2,4]triazolo[1,5- |
| | c]quinazolin-5-yl]amino}-5-oxo-1,4-diazepane-1-carboxylate |
| | LC-MS (Method 2): Rₜ = 1.52 min; MS (ESIpos): m/z = 552 [M + H]⁺ |
| Intermediate 172 | 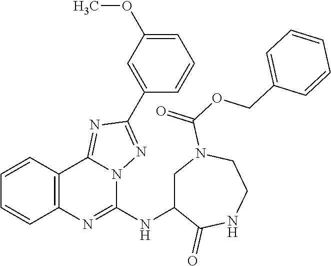 [SMILES: COc1cccc(-c2nc3c4ccccc4nc(NC4CN(C(=O)OCc5ccccc5)CCNC4=O)n3n2)c1] |
| | benzyl 6-{[2-(3-methoxyphenyl)[1,2,4]triazolo[1,5-c]quinazolin-5- |
| | yl]amino}-5-oxo-1,4-diazepane-1-carboxylate |
| | LC-MS (Method 2): Rₜ = 1.38 min; MS (ESIneg): m/z = 536 [M − H]⁻ |

### Intermediate 173

### Ethyl (2-chloro-6-cyanophenyl)carbamate

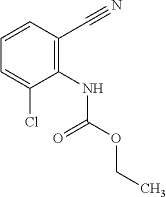

2-Amino-3-chlorobenzonitrile (928 mg, 6.08 mmol) was stirred in ethyl carbonochloridate (9.3 mL, 97 mmol) for 48 h at reflux. The reaction mixture was cooled to rt and concentrated under reduced pressure to give 1.36 g (99% yield) of the title compound that was used without further purification.

LC-MS (method 2): Rₜ=0.89 min; MS (ESIpos): m/z=225 [M+H]⁺

¹H-NMR (400 MHz, DMSO-d₆): δ [ppm]=1.23 (t, 3H), 4.13 (q, 2H), 7.49 (t, 1H), 7.89 (ddd, 2H), 9.71 (br s, 1H).

### Intermediate 174

### Ethyl [2-cyano-6-(trifluoromethyl)phenyl]carbamate

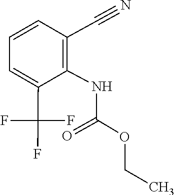 [SMILES: CCOC(=O)Nc1c(C#N)cccc1C(F)(F)F]

2-Amino-3-(trifluoromethyl)benzonitrile (500 mg, 2.69 mmol) was stirred in ethyl carbonochloridate (3.0 mL, 31 mmol) for 7 days at reflux. The mixture cooled to rt and concentrated under reduced pressure to give 621 mg (90% yield) of the title compound that was used without further purification.

LC-MS (method 2): Rₜ=1.01 min; MS (ESIpos): m/z=259 [M+H]⁺

### Intermediate 175

### Ethyl (2-cyano-6-methoxyphenyl)carbamate

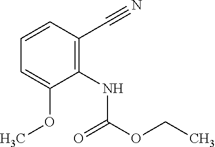 [SMILES: CCOC(=O)Nc1c(C#N)cccc1OC]

2-Amino-3-methoxybenzonitrile (500 mg, 3.37 mmol) was stirred in ethyl carbonochloridate (4.8 mL, 51 mmol) for 18 h at reflux. The reaction mixture was cooled to rt and concentrated under reduced pressure to give 450 mg (61% yield) of the title compound that was used without further purification.

LC-MS (Method 2): Rₜ=0.85 min; MS (ESIpos): m/z=221 [M+H]⁺

¹H NMR (400 MHz, DMSO-d₆) δ ppm=9.13 (br s, 1H), 7.31-7.52 (m, 3H), 4.08 (q, 2H), 3.83 (s, 3H), 1.20 (t, 3H).

### Intermediate 176

### 2-Amino-3-cyclopropylbenzonitrile

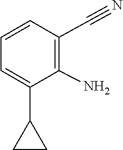 [SMILES: N#Cc1cccc(C2CC2)c1N]

2-Amino-3-bromobenzonitrile (500 mg, 2.54 mmol) was solubilised in 1,4-dioxane (27 mL). cyclopropylboronic acid (262 mg, 3.05 mmol), cesium carbonate (3.31 g, 10.2 mmol) and bis(diphenylphosphino)ferrocene)dichlorpalladium(II)*dichlormethane complex (414 mg, 508 μmol) were added and the reaction was stirred for 10 min at 130° C. under microwave irradiation. The reaction mixture was filtered and the filtrate was concentrated under reduced pressure. The crude material was purified by flash column chromatography to give 275 mg (91% purity, 62% yield) of the title compound.

LC-MS (method 2): Rₜ=1.04 min; MS (ESIpos): m/z=159 [M+H]⁺

### Intermediate 177

### Ethyl (2-cyano-6-cyclopropylphenyl)carbamate

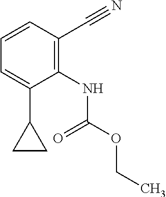 [SMILES: CCOC(=O)Nc1c(C#N)cccc1C1CC1]

2-Amino-3-cyclopropylbenzonitrile (275 mg, 1.74 mmol) was stirred in ethyl carbonochloridate (2.5 mL, 26 mmol) for 4 h at reflux. The reaction mixture was allowed to cool down to rt and concentrated under reduced pressure to give 380 mg (85% purity, 81% yield) of the title compound that was used without further purification.

LC-MS (method 2): Rₜ=1.00 min; MS (ESIneg): m/z=229 [M−H]⁻.

### Intermediate 178

### Ethyl (2-bromo-6-cyanophenyl)carbamate

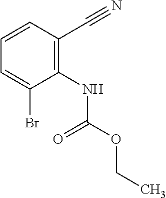

2-Amino-3-bromobenzonitrile (5.00 g, 25.4 mmol) was stirred in ethyl carbonochloridate (29 mL, 300 mmol) for 18 h at reflux. The mixture was cooled to rt and concentrated under reduced pressure to give 6.20 g (84% purity, 76% yield) of the title compound that was used without further purification.

LC-MS (method 2): Rₜ=0.90 min; MS (ESIpos): m/z=269 [M+H]⁺

¹H-NMR (400 MHz, DMSO-d₆): δ [ppm]=1.23 (brt, 3H), 4.13 (q, 2H), 7.41 (t, 1H), 7.91 (dd, 1H), 8.00-8.12 (m, 1H), 9.66 (br s, 1H).

### Intermediate 179

### Ethyl (2,4-dibromo-6-cyanophenyl)carbamate

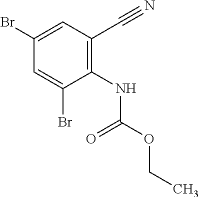 [SMILES: CCOC(=O)Nc1c(Br)cc(Br)cc1C#N]

2-Amino-3,5-dibromobenzonitrile (750 mg, 2.72 mmol) was stirred in ethyl carbonochloridate (5.0 mL, 52 mmol) for 18 h at reflux. The mixture was concentrated under reduced pressure to give 851 mg (66% purity, 59% yield) of the title compound that was used without further purification.

LC-MS (Method 2): Rₜ=1.07 min; MS (ESIpos): m/z=347 [M+H]⁺

¹H-NMR (400 MHz, DMSO-d₆): δ [ppm]=1.29 (t, 3H), 4.19 (q, 2H), 8.32 (d, 1H), 8.42 (d, 1H), 9.78 (br s, 1H).

### Intermediate 180

### Ethyl (2-cyano-5-methoxyphenyl)carbamate

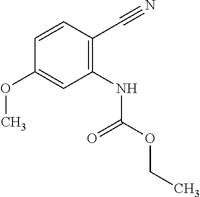 [SMILES: CCOC(=O)Nc1cc(OC)ccc1C#N]

2-Amino-4-methoxybenzonitrile (100 mg, 675 μmol) was stirred in ethyl carbonochloridate (1.4 mL, 15 mmol) for 18 h at 100° C. The reaction mixture was cooled to rt and concentrated under reduced pressure to give 406 mg (74% yield) of the title compound that was used without further purification

LC-MS (Method 2): Rₜ=0.96 min; MS (ESIpos): m/z=221 [M+H]⁺

¹H-NMR (400 MHz, DMSO-d₆): δ [ppm]=1.24 (t, 3H), 3.81 (s, 3H), 4.14 (q, 2H), 6.88 (dd, 1H), 7.09 (d, 1H), 7.70 (d, 1H), 9.66 (s, 1H).

### Intermediate 181

### Ethyl (2-cyano-4,5-dimethoxyphenyl)carbamate

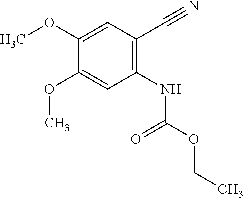 [SMILES: CCOC(=O)Nc1cc(OC)c(OC)cc1C#N]

2-Amino-4,5-dimethoxybenzonitrile (300 mg, 1.68 mmol) was stirred in ethyl carbonochloridate (2.9 mL, 31 mmol) for 6 h at reflux. The reaction mixture was cooled to rt and concentrated under reduced pressure to give 402 mg (100% purity, 95% yield) of the title compound that was used without further purification.

LC-MS (method 2): Rₜ=0.88 min; MS (ESIpos): m/z=251 [M+H]⁺

¹H-NMR (400 MHz, DMSO-d₆): δ [ppm]=1.23 (t, 3H), 3.80 (s, 3H), 3.78 (s, 3H) 4.12 (q, 2H), 7.05 (s, 1H), 7.30 (s, 1H), 9.48 (br s, 1H).

### Intermediate 182

### Ethyl [2-cyano-5-(trifluoromethyl)phenyl]carbamate

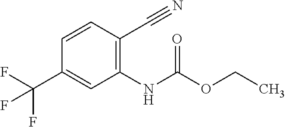 [SMILES: CCOC(=O)Nc1cc(C(F)(F)F)ccc1C#N]

2-Amino-4-(trifluoromethyl)benzonitrile (100 mg, 537 μmol) was stirred with ethyl carbonochloridate (1.0 mL) at 110° C. for 18 h. The reaction mixture was cooled to rt and concentrated under reduced pressure to give 136 mg (98% yield) of the title compound that was used without further purification.

LC-MS (Method 2): Rₜ=1.14 min; MS (ESIneg): m/z=257 [M−H]⁻.

¹H-NMR (400 MHz, DMSO-d₆): δ [ppm]=1.26 (t, 3H), 4.17 (q, 2H), 7.67 (d, 1H), 7.93 (s, 1H), 8.06 (d, 1H), 10.09 (s, 1H).

### Intermediate 183

### Ethyl (5-bromo-2-cyanophenyl)carbamate

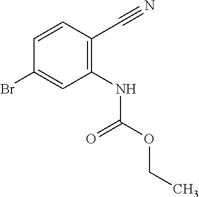

2-Amino-4-bromobenzonitrile (400 mg, 2.03 mmol) was stirred in ethyl carbonochloridate (4.0 mL, 6.1 mmol) for 18 h at 110° C. The reaction mixture was cooled to rt and concentrated under reduced pressure to give 406 mg (74% yield) of the title compound that was used without further purification.

LC-MS (Method 2): Rₜ=1.10 min; MS (ESIpos): m/z=269 [M+H]⁺

¹H-NMR (400 MHz, DMSO-d₆): δ [ppm]=1.25 (t, 3H), 4.16 (q, 2H), 7.54 (dd, 1H), 7.76 (d, 1H), 7.79 (d, 1H), 9.93 (s, 1H).

### Intermediate 184

### Ethyl (2-cyano-4-fluorophenyl)carbamate

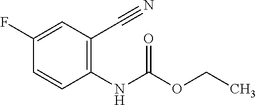

2-Amino-5-fluorobenzonitrile (300 mg, 2.20 mmol) was stirred in ethyl carbonochloridate (8.7 mL) for 6 h at reflux. The mixture was cooled to rt and concentrated under reduced pressure to give 463 mg (99% purity, 100% yield) of the title compound that was used without further purification.

LC-MS (method 2): Rₜ=0.93 min; MS (ESIpos): m/z=207 [M+H]⁺

¹H-NMR (400 MHz, DMSO-d₆): δ [ppm]=1.25 (t, 3H), 4.15 (q, 2H), 7.49-7.55 (m, 1H), 7.55-7.62 (m, 1H), 7.83 (dd, 1H), 9.74 (s, 1H).

### Intermediate 185

### Ethyl (4-chloro-2-cyanophenyl)carbamate

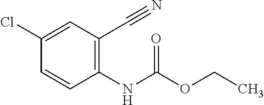

2-Amino-5-chlorobenzonitrile (150 mg, 983 μmol) was stirred in ethyl carbonochloridate (3.9 mL) for 6 h at reflux. The mixture was cooled to rt and concentrated under reduced pressure to give 205 mg (100% purity, 93% yield) of the title compound that was used without further purification.

LC-MS (method 2): Rₜ=1.06 min; MS (ESIneg): m/z=212 [M−H]⁻.

¹H-NMR (400 MHz, DMSO-d₆): δ [ppm]=1.25 (t, 3H), 4.15 (q, 2H), 7.54 (d, 1H), 7.74 (dd, 1H), 7.99 (d, 1H), 9.85 (s, 1H).

### Intermediate 186

### Ethyl (2-cyano-4-methoxyphenyl)carbamate

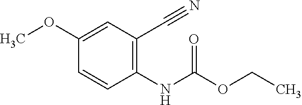 [SMILES: CCOC(=O)Nc1ccc(OC)cc1C#N]

2-Amino-5-methoxybenzonitrile (300 mg, 2.02 mmol) was stirred in ethyl carbonochloridate (8.0 mL) for 6 h at reflux. The mixture was cooled to rt and concentrated under reduced pressure to give 439 mg (96% purity, 94% yield) of the title compound that was used without further purification.

LC-MS (method 2): Rₜ=0.92 min; MS (ESIpos): m/z=221 [M+H]⁺

¹H-NMR (400 MHz, DMSO-d₆): δ [ppm]=1.23 (t, 3H), 3.79 (s, 3H), 4.11 (q, 2H), 7.24 (dd, 1H), 7.35 (d, 1H), 7.38 (d, 1H), 9.47 (br s, 1H).

### Intermediate 187

### Ethyl (4-bromo-2-cyanophenyl)carbamate

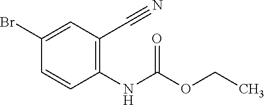

2-Amino-5-bromobenzonitrile (600 mg, 3.05 mmol) was stirred in ethyl carbonochloridate (12 mL) for 6 h at reflux. The mixture was cooled to rt and concentrated under reduced pressure to give 835 mg of the title compound that was used without further purification.

LC-MS (method 2): Rₜ=1.09 min; MS (ESIneg): m/z=267 [M−H]⁻.

¹H-NMR (400 MHz, DMSO-d₆): δ [ppm]=1.25 (t, 3H), 4.15 (q, 2H), 7.47 (d, 1H), 7.86 (dd, 1H), 8.09 (d, 1H), 9.84 (s, 1H).

### Intermediate 188

### Ethyl (2-cyano-3-fluorophenyl)carbamate

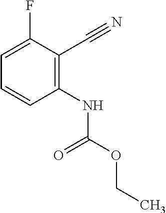

2-Amino-6-fluorobenzonitrile (500 mg, 3.67 mmol) was stirred in ethyl carbonochloridate (7.0 mL, 73 mmol) for 18 h at 100° C. The mixture was cooled to rt and concentrated under reduced pressure to give 755 mg (99% yield) of the title compound that was used without further purification.

LC-MS (method 2): Rₜ=0.96 min; MS (ESIneg): m/z=207 [M−H]⁻.

¹H-NMR (400 MHz, DMSO-d₆): δ [ppm]=1.25 (t, 3H), 4.16 (q, 2H), 7.23-7.32 (m, 1H), 7.38 (d, 1H), 7.72 (td, 1H), 10.00 (s, 1H).

### Intermediate 189

### Ethyl (2-cyano-3-methoxyphenyl)carbamate

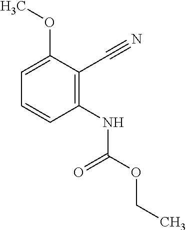 [SMILES: CCOC(=O)Nc1cccc(OC)c1C#N]

2-Amino-6-methoxybenzonitrile (200 mg, 1.35 mmol) was stirred in ethyl carbonochloridate (2.4 mL, 25 mmol) for 18 h at reflux The mixture was cooled to rt and concentrated under reduced pressure to give 276 mg (75% purity, 70% yield) of the title compound that was used without further purification.

LC-MS (method 2): Rₜ=0.94 min; MS (ESIneg): m/z=219 [M−H]⁻.

### Intermediate 190

### Ethyl 1-(propan-2-yl)-1H-pyrazole-4-carboxylate

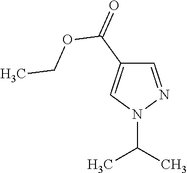

Ethyl 1H-pyrazole-4-carboxylate (100 mg, 714 μmol) was solubilised in anhydrous DMF (1.0 mL) and the mixture was cooled to 0° C. Sodium hydride (37.1 mg, 60% purity in mineral oil, 928 μmol) was added and the reaction mixture was stirred for 15 min at 0° C. 2-Iodopropane (85 μL, 860 μmol) was then added and the mixture was stirred for 18 h at rt. Saturated aqueous sodium hydrogencarbonate was added and the mixture was extracted with ethyl acetate. The organic layer was dried over a silicone filter and concentrated under reduced pressure to give 120 mg (92% yield) of the title compound that was used without further purification.

LC-MS (method 2): Rₜ=0.93 min; MS (ESIpos): m/z=183 [M+H]⁺

¹H-NMR (400 MHz, DMSO-d₆): δ [ppm]=1.26 (t, 3H), 1.41 (d, 6H), 4.20 (q, 2H), 4.55 (spt, 1H), 7.84 (s, 1H), 8.34 (s, 1H).

### Intermediate 191

### 1-(Propan-2-yl)-1H-pyrazole-4-carbohydrazide

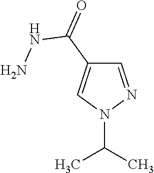

Ethyl 1-(propan-2-yl)-1H-pyrazole-4-carboxylate (60.0 mg, 329 μmol) was solubilised in ethanol (1.2 mL), hydrazine monohydrate (32 μl, 660 μmol) was added and the mixture was stirred for 18 h at rt. The reaction mixture was concentrated under reduced pressure to give 57 mg of the title compound that was used without further purification.

¹H-NMR (400 MHz, DMSO-d₆): δ [ppm]=1.40 (d, 6H), 4.00-4.42 (br s, 2H), 4.49 (spt, 1H), 7.82 (s, 1H), 8.17 (s, 1H), 9.28 (s, 1H).

### Intermediate 192

### 5-Methyl-1,3,4-oxadiazole-2-carbohydrazide

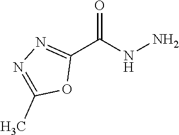

Ethyl 5-methyl-1,3,4-oxadiazole-2-carboxylate (50.0 mg, 320 μmol) and hydrazine hydrate (78 μL, 1.6 mmol) were stirred in ethanol (2.0 mL) for 18 h at rt. The reaction mixture was then poured into water and lyophilised to give 30.0 mg (66% yield) of the title compound that was used without further purification.

### Intermediate 193

### Ethyl 4-chloro-2-(trifluoromethoxy)benzoate

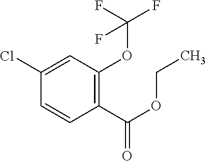 [SMILES: CCOC(=O)c1ccc(Cl)cc1OC(F)(F)F]

4-Chloro-2-(trifluoromethoxy)benzoic acid (1.00 g, 4.16 mmol) in thionyl chloride (1.7 mL, 24 mmol) was stirred for 15 min at rt. At 0° C. ethanol (8.3 mL) was carefully added under strong gas evelution. After 30 min the reaction was stirred at 80° C. for 2.5 h. The reaction was allowed to cool down to rt and concentrated under vacuum. Aqueous saturated sodium hydrogencarbonate solution (25 mL) was added and the aqueous phase was extracted three times with ethyl acetate. The combined organic layers were dried over sodium sulfate and concentrated to give 1.12 g of the title compound which was used without further purification in the next step.

LC-MS (Method 1): Rₜ=1.40 min; MS (ESIpos): m/z=269 [m+H]⁺

¹H-NMR (400 MHz, DMSO-d₆): δ [ppm]=1.30 (t, 3H), 4.33 (q, 2H), 7.68 (dd, 1H), 7.72-7.74 (m, 1H), 7.97 (d, 1H).

### Intermediate 194

### Ethyl 5-chloro-2-(trifluoromethoxy)benzoate

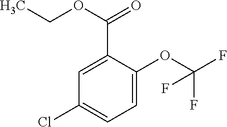 [SMILES: CCOC(=O)c1cc(Cl)ccc1OC(F)(F)F]

5-Chloro-2-(trifluoromethoxy)benzoic acid (370 mg, 1.54 mmol) was dissolved in ethanol (3.5 mL). On an ice bath thionyl chloride (640 μL, 8.8 mmol) was carefully added under gas evolution and stirring was continued for 145 min on the ice bath. It was stirred for two hours under reflux and overnight at rt. The reaction mixture was concentrated under vacuum. The residue was treated twice with dichloromethane and concentrated under reduced pressure obtaining 401 mg (97%) of the title compound which was used without further purification in the next step.

LC-MS (Method 1): Rₜ=1.42 min; MS (ESIpos): m/z=269 [m+H]⁺

¹H-NMR (400 MHz, DMSO-d₆): δ [ppm]=1.30 (t, 3H), 4.33 (q, 2H), 7.58 (qd, 1H), 7.83 (dd, 1H), 7.95 (d, 1H).

### Intermediate 195

### 4-Chloro-2-(trifluoromethoxy)benzohydrazide

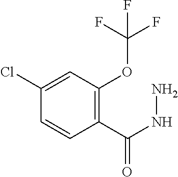 [SMILES: NNC(=O)c1ccc(Cl)cc1OC(F)(F)F]

Ethyl 4-chloro-2-(trifluoromethoxy)benzoate (1.12 g, 4.15 mmol) and hydrazine hydrate (2.0 mL, 42 mmol) in ethanol (18 mL) were stirred at 80° C. for 22 h. The reaction mixture was allowed to cool down to rt and concentrated under reduced pressure. Saturated aqueous ammonium chloride solution (50 mL) was added and it was extracted three time with ethyl acetate. The combined organic layers were dried over sodium sulfate and concentrated to afford 579 mg of the title compound which was used without further purification in the next step.

LC-MS (Method 1): Rₜ=0.84 min; MS (ESIpos): m/z=255 [m+H]⁺

¹H-NMR (400 MHz, DMSO-d₆): δ [ppm]=4.54 (br s, 2H), 7.55-7.57 (m, 2H), 7.60-7.62 (m, 1H), 9.65 (br s, 1H).

### Intermediate 196

### 5-(Trifluoromethyl)pyridine-3-carbohydrazide

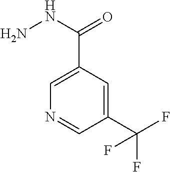 [SMILES: NNC(=O)c1cncc(C(F)(F)F)c1]

Ammonium 5-(trifluoromethyl)nicotinate (500 mg, 2.40 mmol) was suspended in ethanol (3.0 mL). Sulfuric acid (96%, 147 μL, 2.8 mmol) was added and stirred under reflux for 23 h. Thionyl chloride (350 μL, 4.8 mmol) was added and the reaction mixture was stirred under reflux overnight. The reaction mixture was allowed to cool down to rt and hydrazine hydrate (930 μL, 19 mmol) was added and stirred overnight at rt. Then it was stirred under reflux overnight. The reaction mixture was allowed to cool down to rt and concentrated under reduced pressure. Water was added and the aqueous phase was extracted four time with 1-butanol. The combined organic phases were dried over magnesium sulfate and concentrated. The residue was purified by HPLC. The collected aqueous fractions were concentrated under reduced pressure. Dichloromethane was added and evaporated under reduced pressure. This was repeated once more to give 149 mg (30%) of the title compound which was used without further purification in the next step.

LC-MS (Method 2): Rₜ=0.56 min; MS (ESIpos): m/z=206 [m+H]⁺

¹H-NMR (400 MHz, DMSO-d₆): δ [ppm]=4.67 (br s, 2H), 8.52 (s, 1H), 9.11-9.16 (m, 1H), 9.23-9.26 (m, 1H), 10.20 (br s, 1H).

### Intermediate 197

### 3-(Methanesulfonyl)benzohydrazide

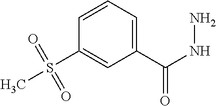

Methyl 3-(methanesulfonyl)benzoate (100 mg, 467 μmol) was dissolved in methanol (2.3 mL). Hydrazine hydrate (114 μL, 2.33 mmol) was added and it was heated at 140° C. for 1 h in a microwave reactor (high absorption). The reaction mixture was allowed to cool down to rt and concentrated under reduced pressure. The residue was partioned between saturated aqueous ammonium chloride solution and ethyl acetate. The layers were separated and the aqueous phase was extracted with ethyl acetate. The combined organic layers were washed with brine, dried over sodium sulfate, concentrated under reduce pressure and dried at 50° C. under vacuum yielding 54 mg (49%) of the title compound which was used without further purification in the next step.

LC-MS (Method 1): Rₜ=0.47 min; MS (ESIpos): m/z=215 [m+H]⁺

¹H-NMR (400 MHz, DMSO-d₆): δ [ppm]=3.26 (s, 3H), 4.60 (br s, 2H), 7.75 (t, 1H), 8.06 (ddd, 1H), 8.13 (ddd, 1H), 8.33 (t, 1H), 10.05 (s, 1H).

### Intermediate 198

### 6-(Trifluoromethyl)pyridine-2-carbohydrazide

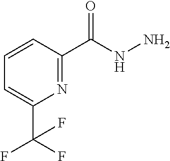 [SMILES: NNC(=O)c1cccc(C(F)(F)F)n1]

Methyl 6-(trifluoromethyl)pyridine-2-carboxylate (648 mg, 3.16 mmol) was dissolved in methanol (15 mL). Hydrazine hydrate (730 μL, 15 mmol) was added and it was heated at 140° C. for 1 h in a microwave reactor (high absorption). The reaction mixture was allowed to cool down to rt and concentrated under reduced pressure. The residue was partioned between saturated aqueous ammonium chloride solution (15 mL) and ethyl acetate (15 mL). The layers were separated and the aqueous phase was extracted with ethyl acetate (twice 15 mL). The combined organic layers were washed with brine (10 mL), dried over magnesium sulfate, and concentrated under reduce pressure yielding 624 of the title compound which was used without further purification in the next step.

LC-MS (Method 2): Rₜ=0.67 min; MS (ESIpos): m/z=206 [m+H]⁺

¹H-NMR (400 MHz, DMSO-d₆): δ [ppm]=4.66 (br s, 2H), 8.08 (dd, 1H), 8.21-8.30 (m, 2H), 9.92 (br s, 1H).

### Intermediate 199

### 5-Chloro-2-(trifluoromethoxy)benzohydrazide

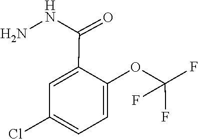 [SMILES: NNC(=O)c1cc(Cl)ccc1OC(F)(F)F]

Ethyl 5-chloro-2-(trifluoromethoxy)benzoate (397 mg, 1.48 mmol) was dissolved in ethanol (3.2 mL) and hydrazine hydrate (360 μL, 7.4 mmol) was added. It was stirred at 90° C. bath temperature for 70 h. The reaction mixture was allowed to cool down to rt and concentrated under reduced pressure. The residue and the residue of a second batch (ethyl 5-chloro-2-(trifluoromethoxy)benzoate, 58 mg, 216 μmol) which was synthesized under similar conditions was added. The combined residue was treated twice with dichloromethane and concentrated under reduce pressure to give 374 mg of the title compound which was used without further purification in the next step.

LC-MS (Method 2): Rₜ=0.84 min; MS (ESIpos): m/z=255 [m+H]⁺

¹H-NMR (400 MHz, DMSO-d₆): δ [ppm]=4.54 (br s, 2H), 7.48 (qd, 1H), 7.58 (d, 1H), 7.66 (dd, 1H), 9.69 (br s, 1H).

### Intermediate 200

### 4-Methoxythiophene-3-carbohydrazide

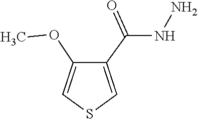

Methyl 4-methoxythiophene-3-carboxylate (938 mg, 4.45 mmol) was dissolved in ethanol (32.3 mL) and hydrazine hydrate (1.33 mL, 27.2 mmol) was added. It was stirred at 85° C. bath temperature for 44 h. The reaction mixture was allowed to cool down to rt and concentrated under reduced pressure to obtain 935 mg (99.7%) of the title compound which was used without further purification in the next step.

LC-MS (Method 2): Rₜ=0.56 min; MS (ESIpos): m/z=173 [m+H]⁺

¹H-NMR (400 MHz, DMSO-d₆): δ [ppm]=3.84 (s, 3H), 4.49 (br s, 2H), 6.73 (d, 1H), 7.96 (d, 1H), 8.76 (s, 1H).

### Intermediate 201

### Thiophene-3-carbohydrazide

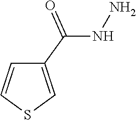

Methyl thiophene-3-carboxylate (800 mg, 5.63 mmol) was dissolved in ethanol (16 mL) and hydrazine hydrate (1.37 mL, 28.1 mmol) was added. It was stirred under reflux for 90 h. The reaction mixture was allowed to cool down to rt and concentrated under reduced pressure. The residue was dissolved in ethanol and concentrated to dryness. This process was repeated to yield 795 mg (94%) of the title compound which was used without further purification in the next step.

¹H-NMR (400 MHz, DMSO-d₆): δ [ppm]=4.46 (br s, 2H), 7.49 (dd, 1H), 7.58 (dd, 1H), 8.09 (dd, 1H), 9.59 (s, 1H).

### Intermediate 202

### 2-Methylthiophene-3-carbohydrazide

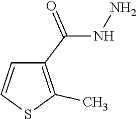

Methyl 2-methylthiophene-3-carboxylate (350 mg, 2.24 mmol) was dissolved in 1-butanol (3.5 mL) and hydrazine hydrate (545 μL, 11.2 mmol) was added. It was stirred at 120° C. bath temperature for 20 h. The reaction mixture was allowed to cool down to rt and concentrated under reduced pressure. The residue was treated with dichloromethane and concentrated under reduced pressure to dryness affording 340 mg (97%) of the title compound which was used without further purification in the next step.

LC-MS (Method 2): Rₜ=0.57 min; MS (ESIpos): m/z=157 [m+H]⁺

¹H-NMR (400 MHz, DMSO-d₆): δ [ppm]=2.62 (s, 3H), 4.80 (br s, 2H), 7.24-7.29 (m, 2H), 9.36 (s, 1H).

### Intermediate 203

### 5-Methylthiophene-3-carbohydrazide

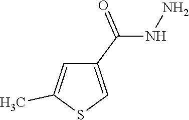

Methyl 5-methylthiophene-3-carboxylate (950 mg, 6.08 mmol) was dissolved in ethanol (9.5 mL) and hydrazine hydrate (1.48 mL, 30.4 mmol) was added. It was stirred under reflux for 6 h and over the weekend at rt. The reaction mixture was concentrated under reduced pressure. The residue was treated with dichloromethane and concentrated to dryness obtaining 830 mg (87%) of the title compound which was used without further purification in the next step.

LC-MS (Method 2): Rₜ=0.61 min; MS (ESIpos): m/z=157 [m+H]⁺

¹H-NMR (400 MHz, DMSO-d₆): δ [ppm]=2.43 (d, 3H), 4.42 (br s, 2H), 7.17 (t, 1H), 7.82 (d, 1H), 9.49 (s, 1H).

### Intermediate 204

### 7-Chloro-2-(pyridin-4-yl)[1,2,4]triazolo[1,5-c]quinazolin-5(6H)-one

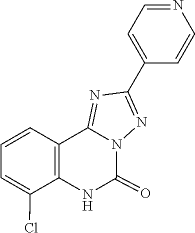 [SMILES: O=c1[nH]c2c(Cl)cccc2c2nc(-c3ccncc3)nn12]

Ethyl (2-chloro-6-cyanophenyl)carbamate (500 mg, 2.23 mmol) and pyridine-4-carbohydrazide (366 mg, 2.67 mmol) were stirred in DMF (24 mL) at 120° C. for 18 h. The reaction was cooled to rt and water was added to the mixture. The suspension was filtered, washed with water and dried under reduced pressure at 60° C. to give 582 mg (88% yield) of the title compound.

LC-MS (method 2): Rₜ=0.55 min; MS (ESIpos): m/z=298 [M+H]⁺

The following intermediates were prepared in analogy to intermediate 204:

| | Structure |
|---|---|
| | IUPAC-Name |
| | LC-MS (method): Retention time; Mass found |
| Intermediate | ¹H-NMR |
| Intermediate 205 | 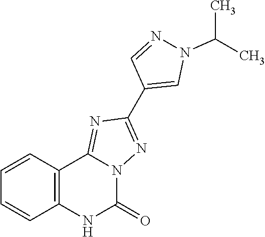 [SMILES: CC(C)n1cc(-c2nc3c4ccccc4[nH]c(=O)n3n2)cn1] |
| | 2-[1-(-2-yl)-1H-pyrazol-4-yl][1,2,4]triazolo[1,5-c]quinazolin-5(6H)-one |
| | LC-MS (Method 2): Rt = 0.59 min; MS (ESIpos): m/z = 295 [M + H]⁺ |
| | ¹H-NMR (400 MHz, DMSO-d₆): δ [ppm] = 1.48 (d, 6H), 4.62 (sept, 1H), 7.39 |
| | (td, 1H), 7.44 (d, 1H), 7.69 (ddd, 1H), 8.03 (s, 1H), 8.16 (dd, 1H), 8.47 (s, |
| | 1H), 12.22 (br s 1H). |
| Intermediate 206 | 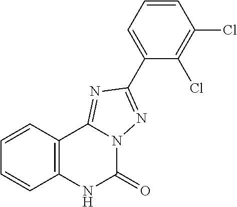 [SMILES: O=c1[nH]c2ccccc2c2nc(-c3cccc(Cl)c3Cl)nn12] |
| | 2-(2,3-dichlorophenyl)[1,2,4]triazolo[1,5-c]quinazolin-5(6H)-one |
| | LC-MS (Method 2): Rₜ = 0.71 min; MS (ESIpos): m/z = 331 [M + H]⁺ |
| Intermediate 207 | 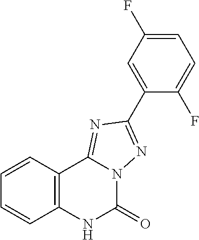 [SMILES: O=c1[nH]c2ccccc2c2nc(-c3cc(F)ccc3F)nn12] |
| | 2-(2,5-difluorophenyl)[1,2,4]triazolo[1,5-c]quinazolin-5(6H)-one |
| | LC-MS (Method 2): Rₜ = 0.65 min; MS (ESIpos): m/z = 299 [M + H]⁺ |
| | ¹H-NMR (400 MHz, DMSO-d₆): δ [ppm] = 7.39-7.56 (m, 4H), 7.73 (ddd, 1H), |
| | 7.94-7.96 (m, 1H), 8.23 (dd, 1H), 12.42 (br s, 1H). |
| Intermediate 208 | 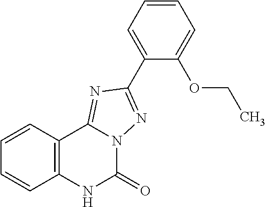 [SMILES: CCOc1ccccc1-c1nc2c3ccccc3[nH]c(=O)n2n1] |
| | 2-(2-ethoxyphenyl)[1,2,4]triazolo[1,5-c]quinazolin-5(6H)-one |
| | LC-MS (Method 2): Rₜ = 0.66 min; MS (ESIpos): m/z = 307 [M + H]⁺ |
| | ¹H-NMR (400 MHz, DMSO-d₆): δ [ppm] = 1.35 (t, 3H), 4.17 (q, 2H), 7.10 (td, |
| | 1H), 7.21 (dd, 1H), 7.37-7.53 (m, 3H), 7.71 (ddd, 1H), 7.91 (dd, 1H), 8.20 |
| | (dd, 1H), 12.30 (br s, 1H). |
| Intermediate 209 | 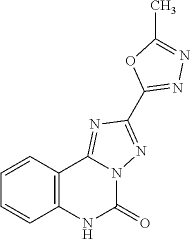 [SMILES: Cc1nnc(-c2nc3c4ccccc4[nH]c(=O)n3n2)o1] |
| | 2-(5-methyl-1,3,4-oxadiazol-2-yl)[1,2,4]triazolo[1,5-c]quinazolin-5(6H)- |
| | one |
| | LC-MS (Method 2): Rₜ = 0.46 min; MS (ESIpos): m/z = 269 [M + H]⁺ |
| | ¹H-NMR (400 MHz, DMSO-d₆) δ [ppm] = 2.68 (s, 3H), 7.41-7.52 (m, 2H), |
| | 7.76 (ddd, 1H), 8.25 (dd, 1H), 12.54 (br s, 1H). |
| Intermediate 210 | 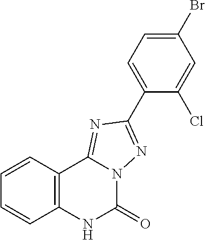 [SMILES: O=c1[nH]c2ccccc2c2nc(-c3ccc(Br)cc3Cl)nn12] |
| | 2-(4-bromo-2-chlorophenyl)[1,2,4]triazolo[1,5-c]quinazolin-5(6H)-one |
| | LC-MS (Method 2): Rₜ = 0.75 min; MS (ESIpos): m/z = 375 [M + H]⁺ |
| | ¹H-NMR (400 MHz, DMSO-d₆): δ [ppm] = 7.39-7.44 (m, 1H), 7.46 (d, 1H), |
| | 7.73 (ddd, 1H), 7.77 (dd, 1H), 7.98 (d, 1H), 8.02 (d, 1H), 8.21 (dd, 1H), |
| | 12.43 (br s, 1H). |
| Intermediate 211 | 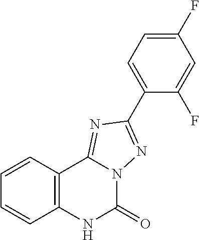 [SMILES: O=c1[nH]c2ccccc2c2nc(-c3ccc(F)cc3F)nn12] |
| | 2-(2,4-difluorophenyl)[1,2,4]triazolo[1,5-c]quinazolin-5(6H)-one |
| | LC-MS (Method 2): Rₜ = 0.65 min; MS (ESIpos): m/z = 299 [M + H]⁺ |
| | ¹H-NMR (400 MHz, DMSO-d₆): δ [ppm] = 7.28-7.35 (m, 1H), 7.38-7.44 (m, |
| | 1H), 7.45 (d, 1H), 7.50 (ddd, 1H), 7.72 (ddd, 1H), 8.21 (dd, 1H), 8.27 (td, |
| | 1H), 12.38 (br s, 1H). |
| Intermediate 212 | 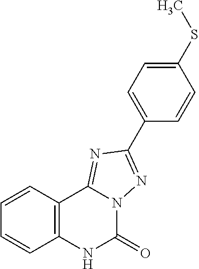 [SMILES: CSc1ccc(-c2nc3c4ccccc4[nH]c(=O)n3n2)cc1] |
| | 2-[4-(methylsulfanyl)phenyl][1,2,4]triazolo[1,5-c]quinazolin-5(6H)-one |
| | LC-MS (Method 2): Rₜ = 0.71 min; MS (ESIpos): m/z = 309 [M + H]⁺ |
| | ¹H-NMR (400 MHz, DMSO-d₆): δ [ppm] = 2.56 (s, 3H), 7.38-7.47 (m, 4H), |
| | 7.71 (ddd, 1H), 8.13-8.18 (m, 2H), 8.23 (dd, 1H), 12.33 (br s, 1H). |
| Intermediate 213 | 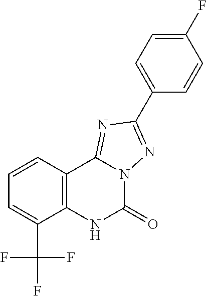 [SMILES: O=c1[nH]c2c(C(F)(F)F)cccc2c2nc(-c3ccc(F)cc3)nn12] |
| | 2-(4-fluorophenyl)-7-(trifluoromethyl)[1,2,4]triazolo[1,5-c]quinazolin- |
| | 5(6H)-one |
| | LC-MS (Method 2): Rₜ = 0.74 min; MS (ESIpos): m/z = 349 [M + H]⁺ |
| Intermediate 214 | 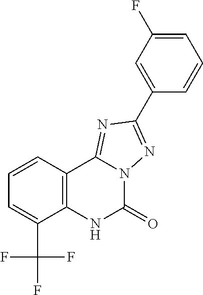 [SMILES: O=c1[nH]c2c(C(F)(F)F)cccc2c2nc(-c3cccc(F)c3)nn12] |
| | 2-(3-fluorophenyl)-7-(trifluoromethyl)[1,2,4]triazolo[1,5-c]quinazolin- |
| | 5(6H)-one |
| | LC-MS (Method 2): Rₜ = 0.75 min; MS (ESIpos): m/z = 349 [M + H]⁺ |
| Intermediate 215 | 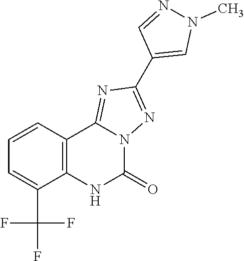 [SMILES: Cn1cc(-c2nc3c4cccc(C(F)(F)F)c4[nH]c(=O)n3n2)cn1] |
| | 2-(1-methyl-1H-pyrazol-4-yl)-7-(trifluoromethyl)[1,2,4]triazolo[1,5- |
| | c]quinazolin-5(6H)-one |
| | LC-MS (Method 2): Rₜ = 0.59 min; MS (ESIpos): m/z = 335 [M + H]⁺ |
| Intermediate 216 | 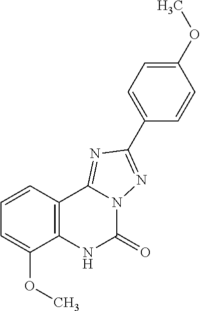 [SMILES: COc1ccc(-c2nc3c4cccc(OC)c4[nH]c(=O)n3n2)cc1] |
| | 7-methoxy-2-(4-methoxyphenyl)[1,2,4]triazolo[1,5-c]quinazolin-5(6H)- |
| | one |
| | LC-MS (Method 2): Rₜ = 0.71 min; MS (ESIpos): m/z = 323 [M + H]⁺ |
| Intermediate 217 | 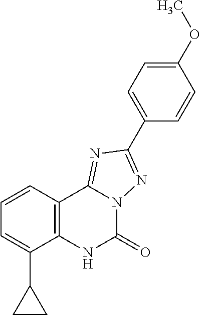 [SMILES: COc1ccc(-c2nc3c4cccc(C5CC5)c4[nH]c(=O)n3n2)cc1] |
| | 7-cyclopropyl-2-(4-methoxyphenyl)[1,2,4]triazolo[1,5-c]quinazolin- |
| | 5(6H)-one |
| | LC-MS (Method 2): Rₜ = 0.84 min; MS (ESIpos): m/z = 333 [M + H]⁺ |
| Intermediate 218 | 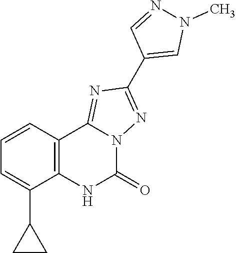 [SMILES: Cn1cc(-c2nc3c4cccc(C5CC5)c4[nH]c(=O)n3n2)cn1] |
| | 7-cyclopropyl-2-(1-methyl-1H-pyrazol-4-yl)[1,2,4]triazolo[1,5- |
| | c]quinazolin-5(6H)-one |
| | LC-MS (Method 2): Rₜ = 0.63 min; MS (ESIpos): m/z = 307 [M + H]⁺ |
| Intermediate 219 | 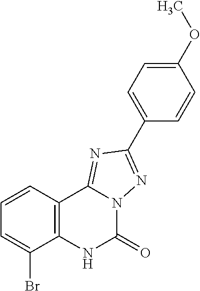 [SMILES: COc1ccc(-c2nc3c4cccc(Br)c4[nH]c(=O)n3n2)cc1] |
| | 7-bromo-2-(4-methoxyphenyl)[1,2,4]triazolo[1,5-c]quinazolin-5(6H)-one |
| | LC-MS (Method 2): Rₜ = 0.69 min; MS (ESIpos): m/z = 371 [M + H]⁺ |
| | ¹H-NMR (400 MHz, DMSO-d₆): δ [ppm] = 3.85 (s, 3H), 7.11-7.16 (m, 2H), |
| | 7.35 (t, 1H), 8.01 (dd, 1H), 8.14-8.20 (m, 2H), 8.26 (dd, 1H), 11.39 (s, 1H). |
| Intermediate 220 | 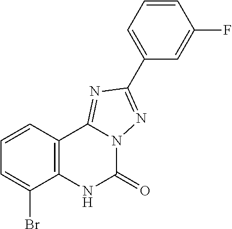 [SMILES: O=c1[nH]c2c(Br)cccc2c2nc(-c3cccc(F)c3)nn12] |
| | 7-bromo-2-(3-fluorophenyl)[1,2,4]triazolo[1,5-c]quinazolin-5(6H)-one |
| | LC-MS (Method 2): Rₜ = 0.72 min; MS (ESIneg): m/z = 357 [M − H]⁻ |
| Intermediate 221 | 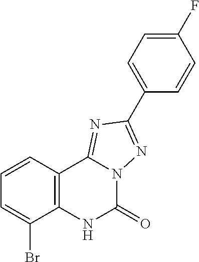 [SMILES: O=c1[nH]c2c(Br)cccc2c2nc(-c3ccc(F)cc3)nn12] |
| | 7-bromo-2-(4-fluorophenyl)[1,2,4]triazolo[1,5-c]quinazolin-5(6H)-one |
| | LC-MS (Method 2): Rₜ = 0.71 min; MS (ESIpos): m/z = 359 [M + H]⁺ |
| Intermediate 222 | 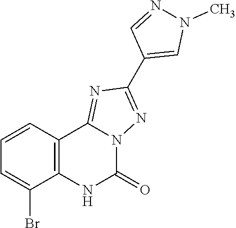 [SMILES: Cn1cc(-c2nc3c4cccc(Br)c4[nH]c(=O)n3n2)cn1] |
| | 7-bromo-2-(1-methyl-1H-pyrazol-4-yl)[1,2,4]triazolo[1,5-c]quinazolin- |
| | 5(6H)-one |
| | LC-MS (Method 2): Rt = 0.47 min; MS (ESIpos): m/z = 345 [M + H]⁺ |
| Intermediate 223 | 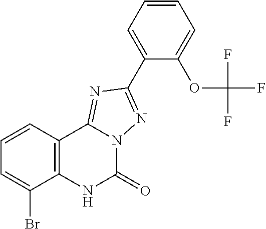 [SMILES: O=c1[nH]c2c(Br)cccc2c2nc(-c3ccccc3OC(F)(F)F)nn12] |
| | 7-bromo-2-[2-(trifluoromethoxy)phenyl][1,2,4]triazolo[1,5-c]quinazolin- |
| | 5(6H)-one |
| | LC-MS (Method 2): Rt = 0.72 min; MS (ESIneg): m/z = 423 [M − H]⁻ |
| Intermediate 224 | 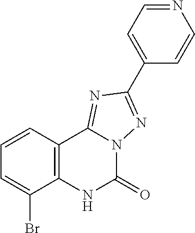 [SMILES: O=c1[nH]c2c(Br)cccc2c2nc(-c3ccncc3)nn12] |
| | 7-bromo-2-(pyridin-4-yl)[1,2,4]triazolo[1,5-c]quinazolin-5(6H)-one |
| | LC-MS (Method 2): Rₜ = 0.56 min; MS (ESIpos): m/z = 342 [M + H]⁺ |
| Intermediate 225 | 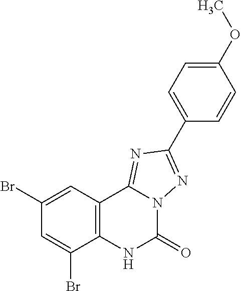 [SMILES: COc1ccc(-c2nc3c4cc(Br)cc(Br)c4[nH]c(=O)n3n2)cc1] |
| | 7,9-dibromo-2-(4-methoxyphenyl)[1,2,4]triazolo[1,5-c]quinazolin-5(6H)- |
| | one |
| | LC-MS (Method 2): Rₜ = 0.78 min; MS (ESIneg): m/z = 449 [M − H]⁻ |
| | ¹H-NMR (400 MHz, DMSO-d₆): δ [ppm] = 3.84-3.87 (m, 3H), 7.10-7.19 (m, |
| | 2H), 8.14-8.21 (m, 2H), 8.24 (d, 1H), 8.33 (d, 1H), 11.58 (br s, 1H). |
| Intermediate 226 | 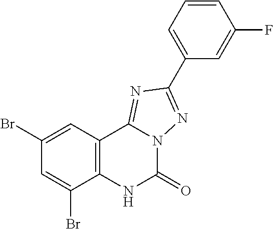 [SMILES: O=c1[nH]c2c(Br)cc(Br)cc2c2nc(-c3cccc(F)c3)nn12] |
| | 7,9-dibromo-2-(3-fluorophenyl)[1,2,4]triazolo[1,5-c]quinazolin-5(6H)- |
| | one |
| | LC-MS (Method 2): Rₜ = 0.80 min; MS (ESIneg): m/z = 437 [M − H]⁻ |
| Intermediate 227 | 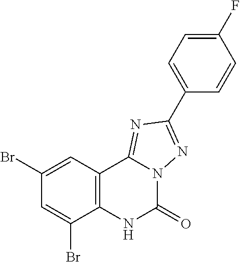 [SMILES: O=c1[nH]c2c(Br)cc(Br)cc2c2nc(-c3ccc(F)cc3)nn12] |
| | 7,9-dibromo-2-(4-fluorophenyl)[1,2,4]triazolo[1,5-c]quinazolin-5(6H)- |
| | one |
| | LC-MS (Method 2): Rₜ = 0.81 min; MS (ESIpos): m/z = 436 [M + H]⁺ |
| Intermediate 228 | 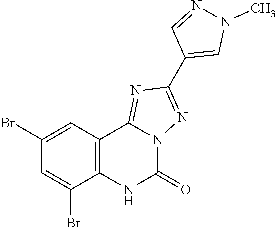 [SMILES: Cn1cc(-c2nc3c4cc(Br)cc(Br)c4[nH]c(=O)n3n2)cn1] |
| | 7,9-dibromo-2-(1-methyl-1H-pyrazol-4-yl)[1,2,4]triazolo[1,5- |
| | c]quinazolin-5(6H)-one |
| | LC-MS (Method 2): Rt = 0.58 min; MS (ESIpos): m/z = 422 [M + H]⁺ |
| | ¹H-NMR (400 MHz, DMSO-d₆): δ [ppm] = 3.87-3.93 (m, 3H), 7.94-8.00 (m, |
| | 2H), 9.84 (s, 1H), 10.15 (s, 1H). |
| Intermediate 229 | 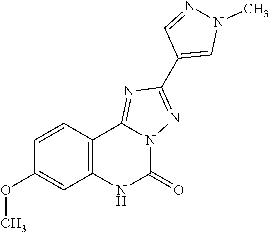 [SMILES: COc1ccc2c(c1)[nH]c(=O)n1nc(-c3cnn(C)c3)nc21] |
| | 8-methoxy-2-(1-methyl-1H-pyrazol-4-yl)[1,2,4]triazolo[1,5-c]quinazolin- |
| | 5(6H)-one |
| | LC-MS (Method 2): Rₜ = 0.52 min; MS (ESIpos): m/z = 297 [M + H]⁺ |
| | ¹H-NMR (400 MHz, DMSO-d₆): δ [ppm] = 3.87 (s, 3H), 3.93 (s, 3H), 6.90 (d, |
| | 1H), 7.00 (dd, 1H), 7.99 (d, 1H), 8.07 (d, 1H), 8.39 (s, 1H). |
| Intermediate 230 | 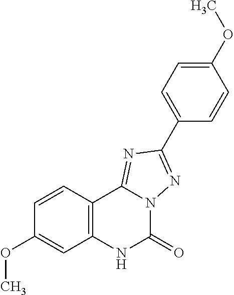 [SMILES: COc1ccc(-c2nc3c4ccc(OC)cc4[nH]c(=O)n3n2)cc1] |
| | 8-methoxy-2-(4-methoxyphenyl)[1,2,4]triazolo[1,5-c]quinazolin-5(6H)- |
| | one |
| | LC-MS (Method 2): Rt = 0.71 min; MS (ESIpos): m/z = 323 [M + H]⁺ |
| | ¹H-NMR (400 MHz, DMSO-d₆): δ [ppm] = 3.87 (s, 3 H), 6.91 (d, |
| | 1H), 7.01 (dd, 1H), 7.08-7.14 (m, 2H), 8.10-8.17 (m, 3H), 12.18 (br s, 1H). |
| Intermediate 231 | 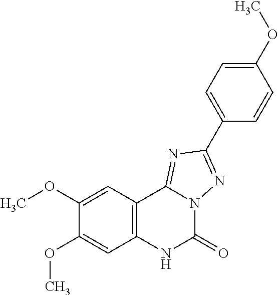 [SMILES: COc1ccc(-c2nc3c4cc(OC)c(OC)cc4[nH]c(=O)n3n2)cc1] |
| | 8,9-dimethoxy-2-(4-methoxyphenyl)[1,2,4]triazolo[1,5-c]quinazolin- |
| | 5(6H)-one |
| | LC-MS (Method 2): Rₜ = 0.63 min; MS (ESIpos): m/z = 353 [M + H]⁺ |
| | ¹H-NMR (400 MHz, DMSO-d₆): δ [ppm] = 3.85 (s, 3H), 3.89 (s, 3H), 3.92 (s, |
| | 3H), 6.96 (s, 1H), 7.08-7.15 (m, 2H), 7.55 (s, 1H), 8.13-8.19 (m, 2H), |
| | 12.11 (br s, 1H). |
| Intermediate 232 | 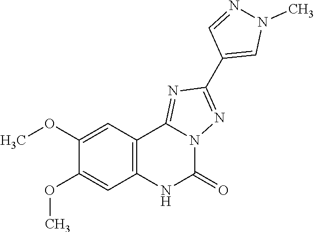 |
| | 8,9-dimethoxy-2-(1-methyl-1H-pyrazol-4-yl)[1,2,4]triazolo[1,5- |
| | c]quinazolin-5(6H)-one |
| | LC-MS (Method 2): Rₜ = 0.48 min; MS (ESIpos): m/z = 327 [M + H]⁺ |
| | ¹H-NMR (400 MHz, DMSO-d₆): δ [ppm] = 3.93 (s, 3 H), 3.90 (s, 3 H), 3.87 (s, |
| | 3 H) 6.95 (s, 1H), 7.48 (s, 1H), 8.00 (d, 1H), 8.40 (s, 1H), 12.07 (br s, 1H). |
| Intermediate 233 | 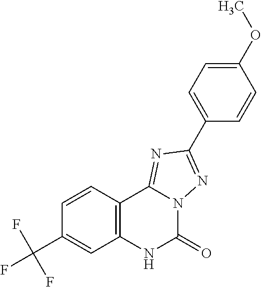 [SMILES: COc1ccc(-c2nc3c4ccc(C(F)(F)F)cc4[nH]c(=O)n3n2)cc1] |
| | 2-(4-methoxyphenyl)-8-(trifluoromethyl)[1,2,4]triazolo[1,5-c]quinazolin- |
| | 5(6H)-one |
| | LC-MS (Method 2): Rt = 0.74 min; MS (ESIpos): m/z = 361 [M + H]⁺ |
| | ¹H-NMR (400 MHz, DMSO-d₆): δ [ppm] = 3.85 (s, 3H), 7.10-7.17 (m, 2H), |
| | 7.69-7.77 (m, 2H), 8.16-8.19 (m, 2H), 8.43 (d, 1H), 12.54 (s, 1H). |
| Intermediate 234 | 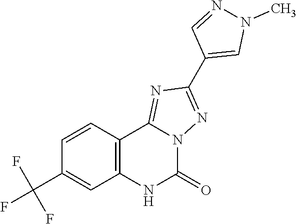 [SMILES: Cn1cc(-c2nc3c4ccc(C(F)(F)F)cc4[nH]c(=O)n3n2)cn1] |
| | 2-(1-methyl-1H-pyrazol-4-yl)-8-(trifluoromethyl)[1,2,4]triazolo[1,5- |
| | c]quinazolin-5(6H)-one |
| | LC-MS (Method 2): Rt = 0.58 min; MS (ESIpos): m/z = 335 [M + H]⁺ |
| Intermediate 235 | 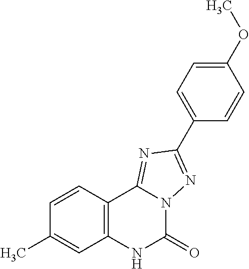 [SMILES: COc1ccc(-c2nc3c4ccc(C)cc4[nH]c(=O)n3n2)cc1] |
| | 2-(4-methoxyphenyl)-8-methyl[1,2,4]triazolo[1,5-c]quinazolin-5(6H)-one |
| | LC-MS (Method 2): Rt = 0.74 min; MS (ESIpos): m/z = 307 [M + H]⁺ |
| | ¹H-NMR (400 MHz, DMSO-d₆): δ [ppm] = 2.45 (s, 3H), 3.85 (s, 3H), 7.09- |
| | 7.14 (m, 2H), 7.21-7.26 (m, 2H), 8.10 (d, 1H), 8.13-8.17 (m, 2H), 12.23 (s, |
| | 1H). |
| Intermediate 236 | 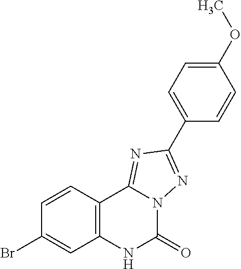 [SMILES: COc1ccc(-c2nc3c4ccc(Br)cc4[nH]c(=O)n3n2)cc1] |
| | 8-bromo-2-(4-methoxyphenyl)[1,2,4]triazolo[1,5-c]quinazolin-5(6H)-one |
| | LC-MS (Method 2): Rt = 0.69 min; MS (ESIpos): m/z = 371 [M + H]⁺ |
| | ¹H-NMR (400 MHz, DMSO-d₆): δ [ppm] = 3.85 (s, 3H), 7.10-7.14 (m, 2H), |
| | 7.55-7.60 (m, 2H), 8.12-8.17 (m, 3H), 12.37 (s, 1H). |
| Intermediate 237 | 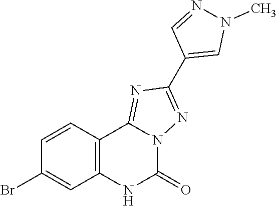 [SMILES: Cn1cc(-c2nc3c4ccc(Br)cc4[nH]c(=O)n3n2)cn1] |
| | 8-bromo-2-(1-methyl-1H-pyrazol-4-yl)[1,2,4]triazolo[1,5-c]quinazolin- |
| | 5(6H)-one |
| | LC-MS (Method 2): Rt = 0.54 min; MS (ESIpos): m/z = 345 [M + H]⁺ |
| | ¹H-NMR (400 MHz, DMSO-d₆): δ [ppm] = 3.93 (s, 3H), 7.56 (dd, 1H), 7.58 (d, |
| | 1H), 8.01 (s, 1H), 8.08 (d, 1H), 8.41 (s, 1H), 12.33 (br s, 1H). |
| Intermediate 238 | 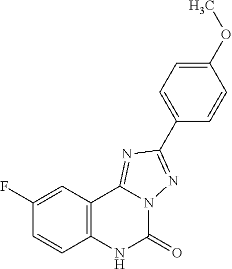 [SMILES: COc1ccc(-c2nc3c4cc(F)ccc4[nH]c(=O)n3n2)cc1] |
| | 9-fluoro-2-(4-methoxyphenyl)[1,2,4]triazolo[1,5-c]quinazolin-5(6H)-one |
| | LC-MS (Method 2): Rₜ = 0.66 min; MS (ESIneg): m/z = 309 [M − H]⁻ |
| | ¹H-NMR (400 MHz, DMSO-d₆): δ [ppm] = 3.85 (s, 3H), 7.07-7.17 (m, 2H), |
| | 7.47 (dd, 1H), 7.61 (td, 1H), 7.95 (dd, 1H), 8.12-8.21 (m, 2H), 12.35 (br s, |
| | 1H). |
| Intermediate 239 | 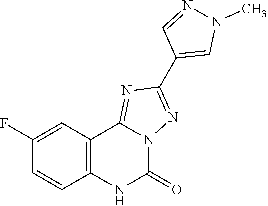 [SMILES: Cn1cc(-c2nc3c4cc(F)ccc4[nH]c(=O)n3n2)cn1] |
| | 9-fluoro-2-(1-methyl-1H-pyrazol-4-yl)[1,2,4]triazolo[1,5-c]quinazolin- |
| | 5(6H)-one |
| | LC-MS (Method 5): Rₜ = 1.19 min; MS (ESIneg): m/z = 283 [M − H]⁻ |
| Intermediate 240 | 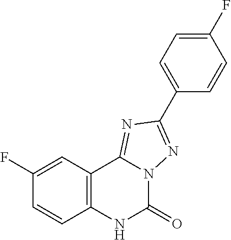 [SMILES: O=c1[nH]c2ccc(F)cc2c2nc(-c3ccc(F)cc3)nn12] |
| | 9-fluoro-2-(4-fluorophenyl)[1,2,4]triazolo[1,5-c]quinazolin-5(6H)-one |
| | LC-MS (Method 2): Rₜ = 0.67 min; MS (ESIneg): m/z = 297 [M − H]⁻ |
| | ¹H-NMR (400 MHz, DMSO-d₆): δ [ppm] = 7.38-7.45 (m, 2H), 7.48 (dd, 1H), |
| | 7.62 (td, 1H), 7.96 (dd, 1H), 8.21-8.31 (m, 2H), 12.41 (br s, 1H). |
| Intermediate 241 | 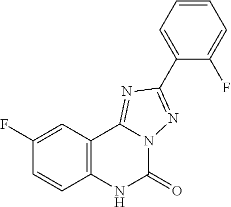 [SMILES: O=c1[nH]c2ccc(F)cc2c2nc(-c3ccccc3F)nn12] |
| | 9-fluoro-2-(2-fluorophenyl)[1,2,4]triazolo[1,5-c]quinazolin-5(6H)-one |
| | LC-MS (Method 2): Rₜ = 0.64 min; MS (ESIneg): m/z = 297 [M − H]⁻ |
| Intermediate 242 | 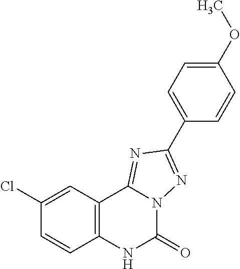 [SMILES: COc1ccc(-c2nc3c4cc(Cl)ccc4[nH]c(=O)n3n2)cc1] |
| | 9-chloro-2-(4-methoxyphenyl)[1,2,4]triazolo[1,5-c]quinazolin-5(6H)-one |
| | LC-MS (Method 2): Rₜ = 0.72 min; MS (ESIpos): m/z = 327 [M + H]⁺ |
| | ¹H-NMR (400 MHz, DMSO-d₆): δ [ppm] = 3.85 (s, 3H), 7.10-7.15 (m, 2H), |
| | 7.45 (d, 1H), 7.75 (dd, 1H), 8.13-8.17 (m, 3H), 12.42 (br s, 1H). |
| Intermediate 243 | 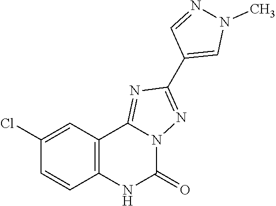 [SMILES: Cn1cc(-c2nc3c4cc(Cl)ccc4[nH]c(=O)n3n2)cn1] |
| | 9-chloro-2-(1-methyl-1H-pyrazol-4-yl)[1,2,4]triazolo[1,5-c]quinazolin- |
| | 5(6H)-one |
| | LC-MS (Method 2): Rₜ = 0.54 min; MS (ESIpos): m/z = 301 [M + H]⁺ |
| Intermediate 244 | 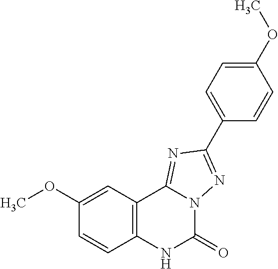 [SMILES: COc1ccc(-c2nc3c4cc(OC)ccc4[nH]c(=O)n3n2)cc1] |
| | 9-methoxy-2-(4-methoxyphenyl)[1,2,4]triazolo[1,5-c]quinazolin-5(6H)- |
| | one |
| | LC-MS (Method 2): Rₜ = 0.66 min; MS (ESIneg): m/z = 321 [M − H]⁻ |
| | ¹H-NMR (400 MHz, DMSO-d₆): δ [ppm] = 3.85 (s, 3H), 3.90 (s, 3H), 7.12 (d, |
| | 2H), 7.30-7.36 (m, 1H), 7.37-7.41 (m, 1H), 7.62 (d, 1H), 8.12-8.21 (m, |
| | 2H), 12.19 (s, 1H). |
| Intermediate 245 | 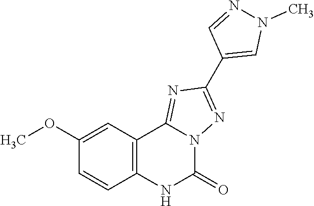 |
| | 9-methoxy-2-(1-methyl-1H-pyrazol-4-yl)[1,2,4]triazolo[1,5-c]quinazolin- |
| | 5(6H)-one |
| | LC-MS (Method 2): Rₜ = 0.51 min; MS (ESIneg): m/z = 295 [M − H]⁻ |
| | ¹H-NMR (400 MHz, DMSO-d₆): δ [ppm] = 3.88 (s, 3H), 3.94 (s, 3H), 7.30- |
| | 7.36 (m, 1H), 7.36-7.40 (m, 1H), 7.55 (d, 1H), 8.02 (s, 1H), 8.43 (s, 1H), |
| | 12.15 (br s, 1H). |
| Intermediate 246 | 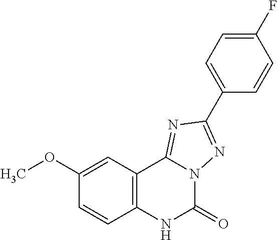 [SMILES: COc1ccc2[nH]c(=O)n3nc(-c4ccc(F)cc4)nc3c2c1] |
| | 2-(4-fluorophenyl)-9-methoxy[1,2,4]triazolo[1,5-c]quinazolin-5(6H)-one |
| | LC-MS (Method 2): Rₜ = 0.68 min; MS (ESIneg): m/z = 309 [M − H]⁻ |
| | ¹H-NMR (400 MHz, DMSO-d₆): δ [ppm] = 3.90 (s, 3H), 7.31-7.47 (m, 4H), |
| | 7.61 (d, 1H), 8.22-8.31 (m, 2H), 12.24 (s, 1H). |
| Intermediate 247 | 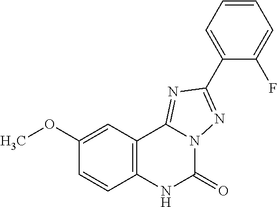 [SMILES: COc1ccc2[nH]c(=O)n3nc(-c4ccccc4F)nc3c2c1] |
| | 2-(2-fluorophenyl)-9-methoxy[1,2,4]triazolo[1,5-c]quinazolin-5(6H)-one |
| | LC-MS (Method 2): Rt = 0.63 min; MS (ESIpos): m/z = 311 [M + H]⁺ |
| | ¹H-NMR (400 MHz, DMSO-d₆): δ [ppm] = 3.90 (s, 3H), 7.32-7.48 (m, 4H), |
| | 7.57-7.67 (m, 2H), 8.23 (td, 1H), 12.27 (s, 1H). |
| Intermediate 248 | 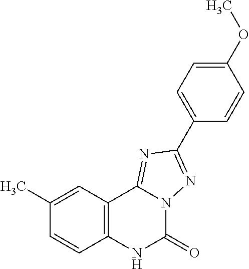 [SMILES: COc1ccc(-c2nc3c4cc(C)ccc4[nH]c(=O)n3n2)cc1] |
| | 2-(4-methoxyphenyl)-9-methyl[1,2,4]triazolo[1,5-c]quinazolin-5(6H)-one |
| | LC-MS (Method 2): Rt = 0.74 min; MS (ESIpos): m/z = 307 [M + H]⁺ |
| | ¹H-NMR (400 MHz, DMSO-d₆): δ [ppm] = 2.44 (s, 3H), 3.85 (s, 3H), 7.10- |
| | 7.15 (m, 2H), 7.34 (d, 1H), 7.53 (dd, 1H), 8.02 (d, 1H), 8.13-8.18 (m, 2H), |
| | 12.22 (br s, 1H). |
| Intermediate 249 | 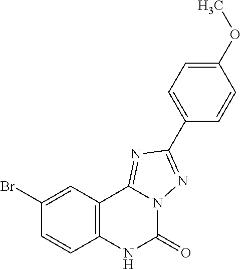 [SMILES: COc1ccc(-c2nc3c4cc(Br)ccc4[nH]c(=O)n3n2)cc1] |
| | 9-bromo-2-(4-methoxyphenyl)[1,2,4]triazolo[1,5-c]quinazolin-5(6H)-one |
| | LC-MS (Method 2): Rₜ = 0.73 min; MS (ESIneg): m/z = 369 [M − H]⁻ |
| | ¹H-NMR (400 MHz, DMSO-d₆): δ [ppm] = 3.85 (s, 3H), 7.09-7.17 (m, 2H), |
| | 7.39 (d, 1H), 7.87 (dd, 1H), 8.12-8.19 (m, 2H), 8.29 (d, 1H), 12.42 (br s, |
| | 1H). |
| Intermediate 250 | 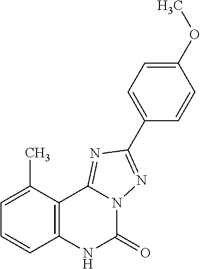 [SMILES: COc1ccc(-c2nc3c4c(C)cccc4[nH]c(=O)n3n2)cc1] |
| | 2-(4-methoxyphenyl)-10-methyl[1,2,4]triazolo[1,5-c]quinazolin-5(6H)- |
| | one |
| | LC-MS (Method 2): Rₜ = 0.89 min; MS (ESIpos): m/z = 307 [M + H]⁺ |
| | ¹H-NMR (400 MHz, DMSO-d₆): δ [ppm] = 2.96 (s, 3H), 3.85 (s, 3H), 7.10- |
| | 7.15 (m, 2H), 7.24 (d, 1H), 7.29 (d, 1H), 7.53-7.60 (m, 1H), 8.14-8.19 (m, |
| | 2H), 12.24 (br s, 1H). |
| Intermediate 251 | 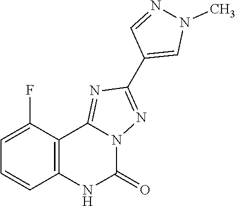 [SMILES: Cn1cc(-c2nc3c4c(F)cccc4[nH]c(=O)n3n2)cn1] |
| | 10-fluoro-2-(1-methyl-1H-pyrazol-4-yl)[1,2,4]triazolo[1,5-c]quinazolin- |
| | 5(6H)-one |
| | LC-MS (Method 2): Rₜ = 0.46 min; MS (ESIpos): m/z = 285 [M + H]⁺ |
| | ¹H-NMR (400 MHz, DMSO-d₆): δ [ppm] = 3.94 (s, 3H), 7.19-7.28 (m, 2H), |
| | 7.69 (td, 1H), 8.00 (d, 1H), 8.43 (s, 1H), 12.43 (br s, 1H). |
| Intermediate 252 | 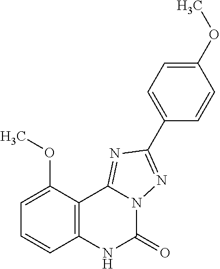 [SMILES: COc1ccc(-c2nc3c4c(OC)cccc4[nH]c(=O)n3n2)cc1] |
| | 10-methoxy-2-(4-methoxyphenyl)[1,2,4]triazolo[1,5-c]quinazolin-5(6H)- |
| | one |
| | LC-MS (Method 2): Rₜ = 0.60 min, MS (ESIpos): m/z = 323 [M + H]⁺ |
| | ¹H-NMR (400 MHz, DMSO-d₆): δ [ppm] = 3.85 (s, 3H), 4.02 (s, 3H), 6.97- |
| | 7.03 (m, 2H), 7.10-7.15 (m, 2H), 7.62 (t, 1H), 8.13-8.18 (m, 2H), 12.24 (br |
| | s, 1H). |

### Intermediate 253

### 2-(4-Methoxyphenyl)-5-oxo-5,6-dihydro[1,2,4]triazolo[1,5-c]quinazoline-10-carbonitrile

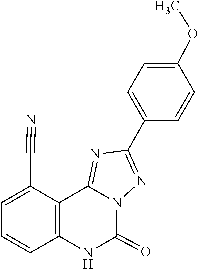 [SMILES: COc1ccc(-c2nc3c4c(C#N)cccc4[nH]c(=O)n3n2)cc1]

10-Bromo-2-(4-methoxyphenyl)[1,2,4]triazolo[1,5-c]quinazolin-5(6H)-one (50 mg, 0.14 mmol), bis[cinnamyl palladium(II) chloride] (3.5 mg, 0.007 mmol), 1,1′-ferrocenediyl-bis(diphenylphosphine) (3.7 mg, 0.007 mmol) and zinc cyanide (15.8 mg, 0.14 mmol) were added to a 5 ml reaction vessel and the vessel sealed and flushed with argon. Degassed N,N-dimethylacetamid (1 ml) and N,N-diisopropylethylamin (47 μl 0.27 mmol), were added and the mixture heated overnight at 80° C. The mixture was cooled to RT, the precipitate filtered and washed with water. The solid material was dissolved in DCM and flushed though a 2 g silica column, and the column washed with a DCM:MeOH mixture (9:1), the eluent was collected and the solvent removed under reduced pressure yielding the title compound (43.9 mg, 0.12 mmol, 87%).

LC-MS (method 1): Rₜ=1.00 min; MS (ESIpos): m/z=318.3 [M+H]⁺

¹H-NMR (400 MHz, DMSO-d₆): δ 3.85 (s, 3H) 7.13-7.20 (m, 2H) 7.70 (d, 1H) 7.76-8.02 (m, 2H) 8.15-8.21 (m, 2H)

### Intermediate 254

### 2-(1-Methyl-1H-pyrazol-4-yl)-5-oxo-5,6-dihydro[1,2,4]triazolo[1,5-c]quinazoline-10-carbonitrile

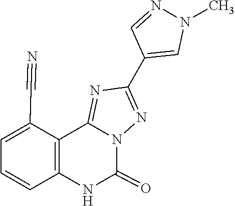 [SMILES: Cn1cc(-c2nc3c4c(C#N)cccc4[nH]c(=O)n3n2)cn1]

10-Bromo-2-(1-methyl-1H-pyrazol-4-yl)[1,2,4]triazolo[1,5-c]quinazolin-5(6H)-one (177 mg, 0.51 mmol), bis[cinnamyl palladium(II) chloride] (13.2 mg, 0.026 mmol), 1,1′-ferrocenediyl-bis(diphenylphosphine) (14.2 mg, 0.026 mmol) and zinc cyanide (60.2 mg, 0.51 mmol) were added to a 5 ml reaction vessel and the vessel sealed and flushed with argon. Degassed N,N-dimethylacetamid (2 ml) and N,N-diisopropylethylamin (179 μl, 1.03 mmol), were added and the mixture heated overnight at 80° C. The mixture was cooled to RT, the mixture was diluted with DCM, washed with NaHCO₃ (saturated aqueous solution), and the aqeuous phase extracted twice with DCM. The combined organic phases were dried passed through a water repellent filter, and purified by RP-HPLC (column: X-Bridge C18 5μ 100×30 mm; acetonitrile/water+0.1% formic acid) yielding the title compound (32 mg, 0.10 mmol, 19%).

LC-MS (method 1): Rₜ=1.00 min; MS (ESIpos): m/z=292.2 [M+H]⁺

¹H-NMR (400 MHz, DMSO-d₆): δ 3.96 (s, 3H) 7.70 (dd, 1H) 7.81 (t, 1H) 7.88 (d, 1H) 7.99 (d, 1H) 8.38 (s, 1H) 12.47-12.70 (m, 1H)

### Intermediate 255

### 2-(4-Fluorophenyl)-5-oxo-5,6-dihydro[1,2,4]triazolo[1,5-c]quinazoline-10-carbonitrile

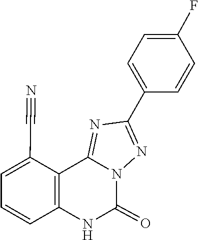 [SMILES: N#Cc1cccc2[nH]c(=O)n3nc(-c4ccc(F)cc4)nc3c12]

10-Bromo-2-(4-fluorophenyl)[1,2,4]triazolo[1,5-c]quinazolin-5(6H)-one (157 mg, 0.43 mmol), bis[cinnamyl palladium(II) chloride] (11.3 mg, 0.022 mmol), 1,1′-ferrocenediyl-bis(diphenylphosphine) (12.1 mg, 0.022 mmol) and zinc cyanide (51.3 mg, 0.44 mmol) were added to a 5 ml reaction vessel and the vessel sealed and flushed with argon. Degassed N,N-dimethylacetamid (2 ml) and N,N-diisopropylethylamin (152 μl, 0.87 mmol), were added and the mixture heated overnight at 80° C. The mixture was cooled to RT, the mixture was diluted with DCM, washed with NaHCO₃ (saturated aqueous solution), and the aqueous phase extracted twice with DCM. The combined organic phases were dried passed through a water repellent filter, and purified by RP-HPLC (column: X-Bridge C18 5μ 100×30 mm; acetonitrile/water+0.1% formic acid), yielding the title compound (53 mg, 0.16 mmol, 36%).

LC-MS (method 1): Rₜ=1.02 min; MS (ESIpos): m/z=306.1 [M+H]⁺

¹H-NMR (400 MHz, DMSO-d₆): δ 7.39-7.50 (m, 2H) 7.73 (d, 1H) 7.81-7.94 (m, 2H) 8.28 (dd, 2H) 12.54-12.79 (m, 1H)

### Intermediate 256

### 2-(3-Fluorophenyl)-5-oxo-5,6-dihydro[1,2,4]triazolo[1,5-c]quinazoline-10-carbonitrile

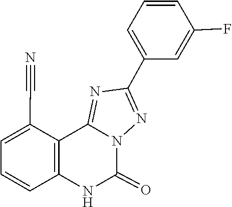 [SMILES: N#Cc1cccc2[nH]c(=O)n3nc(-c4cccc(F)c4)nc3c12]

10-Bromo-2-(3-fluorophenyl)[1,2,4]triazolo[1,5-c]quinazolin-5(6H)-one (152 mg, 0.43 mmol), bis[cinnamyl palladium(II) chloride] (11.0 mg, 0.021 mmol), 1,1′-ferrocenediyl-bis(diphenylphosphine) (11.7 mg, 0.021 mmol) and zinc cyanide (50.0 mg, 0.43 mmol) were added to a 5 ml reaction vessel and the vessel sealed and flushed with argon. Degassed N,N-dimethylacetamid (2 ml) and N,N-diisopropylethylamin (147 μl, 0.74 mmol), were added and the mixture heated overnight at 80° C. The mixture was cooled to RT, the mixture was diluted with DCM, washed with NaHCO₃ (saturated aqueous solution), and the aqueous phase extracted twice with DCM. The combined organic phases were dried passed through a water repellent filter, and purified by RP-HPLC (column: X-Bridge C18 5μ 100×30 mm; acetonitrile/water+0.1% formic acid), yielding the title compound (77 mg, 0.21 mmol, 50%).

LC-MS (method 1): Rₜ=1.02 min; MS (ESIpos): m/z=306.1 [M+H]⁺

¹H-NMR (400 MHz, DMSO-d₆): δ ppm 7.40-7.48 (m, 1H) 7.65-7.76 (m, 2H) 7.83-7.95 (m, 3H) 8.09 (dt, J=7.98, 1.08 Hz, 1H) 12.72 (br s, 1H)

### Intermediate 257

### 2-[2-(Trifluoromethoxy)phenyl][1,2,4]triazolo[1,5-c]quinazolin-5(6H)-one

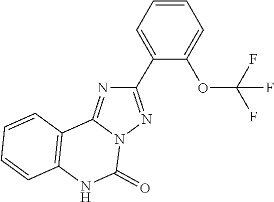 [SMILES: O=c1[nH]c2ccccc2c2nc(-c3ccccc3OC(F)(F)F)nn12]

Methyl (2-cyanophenyl)carbamate (800 mg, 4.54 mmol) and 2-(trifluoromethoxy)benzohydrazide (1.00 g, 4.54 mmol) were dissolved in DMF (16 mL). It was stirred at 120° C. for 72 h and at 130° C. for 96 h. The reaction mixture was allowed to cool down to rt, water was added (20 mL) and it was stirred for 15 minutes. The precipitate was filtered off, washed twice with water and dried under vacuum at 50° C. to afford 1.05 g (67%) of the title compound which was used without further purification in the next step.

LC-MS (Method 1): Rₜ=1.15 min; MS (ESIpos): m/z=347 [m+H]⁺

¹H-NMR (400 MHz, DMSO-d₆): δ [ppm]=7.40-7.48 (m, 2H), 7.58-7.65 (m, 2H), 7.68-7.75 (m, 2H), 8.20 (dd, 1H), 8.28 (dd, 1H), 12.41 (br s, 1H).

The following intermediates were prepared analogously to intermediate 256:

| | Structure |
|---|---|
| | IUPAC-Name |
| | LC-MS (method): Retention time; Mass found |
| Intermediate | ¹H-NMR |
| Intermediate 258 | 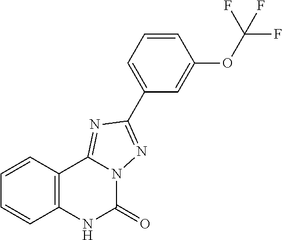 [SMILES: O=c1[nH]c2ccccc2c2nc(-c3cccc(OC(F)(F)F)c3)nn12] |
| | 2-[3-(trifluoromethoxy)phenyl][1,2,4]triazolo[1,5-c]quinazolin-5(6H)-one |
| | LC-MS (Method 2): Rₜ = 0.83 min; MS (ESIpos): m/z = 347 [m + H]⁺ |
| | ¹H-NMR (400 MHz, DMSO-d₆): δ [ppm] = 7.40 − 7.50 (m, 2H), 7.56 − 7.60 (m, |
| | 1H), 7.71 − 7.77 (m, 2H), 8.08 − 8.11 (m, 1H), 8.23 − 8.28 (m, 2H), 12.40 (s, |
| | 1H). |
| Intermediate 259 | 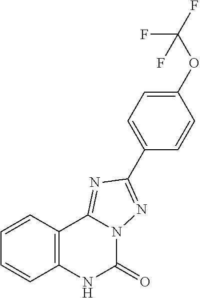 [SMILES: O=c1[nH]c2ccccc2c2nc(-c3ccc(OC(F)(F)F)cc3)nn12] |
| | 2-[4-(trifluoromethoxy)phenyl][1,2,4]triazolo[1,5-c]quinazolin-5(6H)-one |
| | LC-MS (Method 1): Rₜ= 1.23 min; MS (ESIpos): m/z = 347 [m + H]⁺ |
| | ¹H-NMR (400 MHz, DMSO-d₆): δ [ppm] = 7.40 − 7.45 (m, 1H), 7.46 (d, 1H), |
| | 7.55 − 7.61 (m, 2H), 7.73 (ddd, 1H), 8.24 (dd, 1H), 8.33 − 8.37 (m, 2H), 12.38 |
| | (br s, 1H). |
| Intermediate 260 | 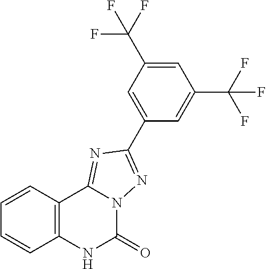 [SMILES: O=c1[nH]c2ccccc2c2nc(-c3cc(C(F)(F)F)cc(C(F)(F)F)c3)nn12] |
| | 2-[3,5-bis(trifluoromethyl)phenyl][1,2,4]triazolo[1,5-c]quinazolin-5(6H)- |
| | one |
| | LC-MS (Method 1): Rₜ = 0.92 min; MS (ESIneg): m/z = 397 [m − H]⁻ |
| | ¹H-NMR (400 MHz, DMSO-d₆): δ [ppm] = 7.40 − 7.45 (m, 1H), 7.47 (d, 1H), |
| | 7.74 (ddd, 1H), 8.29 (dd, 1H), 8.37 (s, 1H), 8.72 (s, 2H), 12.45 (br s, 1H). |
| Intermediate 261 | 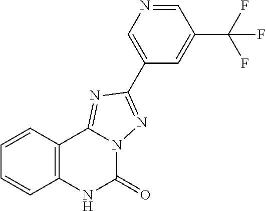 [SMILES: O=c1[nH]c2ccccc2c2nc(-c3cncc(C(F)(F)F)c3)nn12] |
| | 2-[5-(trifluoromethyl)pyridin-3-yl][1,2,4]triazolo[1,5-c]quinazolin-5(6H)- |
| | one |
| | LC-MS (Method 2): Rₜ = 0.69 min; MS (ESIpos): m/z = 332 [m + H]⁺ |
| | ¹H-NMR (400 MHz, DMSO-d₆): δ [ppm] = 7.42 − 7.46 (m, 1H), 7.48 (d, 1H), |
| | 7.75 (ddd, 1H), 8.28 (dd, 1H), 8.76 − 8.79 (m, 1H), 9.19 (d, 1H), 9.64 (d, 1H), |
| | 12.48 (s, 1H). |
| Intermediate 262 | 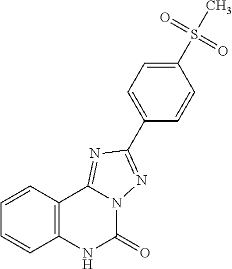 [SMILES: CS(=O)(=O)c1ccc(-c2nc3c4ccccc4[nH]c(=O)n3n2)cc1] |
| | 2-[4-(methanesulfonyl)phenyl][1,2,4]triazolo[1,5-c]quinazolin-5(6H)-one |
| | LC-MS (Method 2): Rₜ = 0.58 min; MS (ESIpos): m/z = 341 [m + H]⁺ |
| | ¹H-NMR (400 MHz, DMSO-d₆): δ [ppm] = 3.31 (s, 3H), 7.41 − 7.50 (m, 2H), |
| | 7.71 − 7.77 (m, 1H), 8.14 (d, 2H), 8.26 (d, 1H), 8.48 (d, 2H), 12.42 (s, 1H). |
| Intermediate 263 | 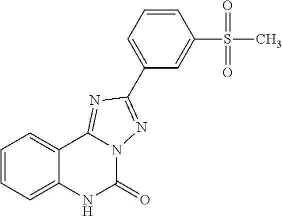 [SMILES: CS(=O)(=O)c1cccc(-c2nc3c4ccccc4[nH]c(=O)n3n2)c1] |
| | 2-[3-(methanesulfonyl)phenyl][1,2,4]triazolo[1,5-c]quinazolin-5(6H)-one |
| | LC-MS (Method 2): Rₜ = 0.85 min; MS (ESIpos): m/z = 341 [m + H]⁺ |
| | ¹H-NMR (400 MHz, DMSO-d₆): δ [ppm] = 3.33 (s, 3H), 7.41 − 7.45 (m, 1H), |
| | 7.47 (d, 1H), 7.74 (ddd, 1H), 7.89 (t, 1H), 8.12 (ddd, 1H), 8.28 (dd, 1H), 8.56 |
| | (dt, 1H), 8.71 (t, 1H), 12.42 (br s, 1H). |
| Intermediate 264 | 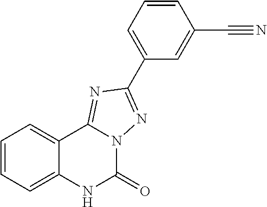 [SMILES: N#Cc1cccc(-c2nc3c4ccccc4[nH]c(=O)n3n2)c1] |
| | 3-(5-oxo-5,6-dihydro[1,2,4]triazolo[1,5-c]quinazolin-2-yl)benzonitrile |
| | LC-MS (Method 1): Rt = 0.97 min; MS (ESIpos): m/z = 288 [m + H]⁺ |
| | ¹H-NMR (400 MHz, DMSO-d6): δ [ppm] = 7.40 − 7.49 (m, 2H), 7.73 (ddd, 1H), |
| | 7.81 (td, 1H), 8.03 (dt, 1H), 8.2-[ (dd, 1H), 8.50 − 8.55 (m, 2H), 12.40 (br s, |
| | 1H). |
| Intermediate 265 | 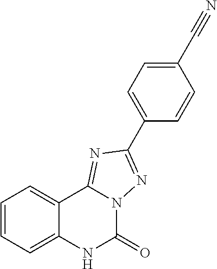 [SMILES: N#Cc1ccc(-c2nc3c4ccccc4[nH]c(=O)n3n2)cc1] |
| | 4-(5-oxo-5,6-dihydro[1,2,4]triazolo[1,5-c]quinazolin-2-yl)benzonitrile |
| | LC-MS (Method 1): Rₜ = 0.97 min; MS (ESIpos): m/z = 288 [m + H]⁺ |
| | ¹H-NMR (400 MHz, DMSO-d₆): δ [ppm] = 7.40 − 7.48 (m, 2H), 7.71 − 7.76 (m, |
| | 1H), 8.01 − 8.09 (m, 2H), 8.2-[ (dd, 1H), 8.35 − 8.42 (m, 2H), 12.42 (br s, 1H). |
| Intermediate 266 | 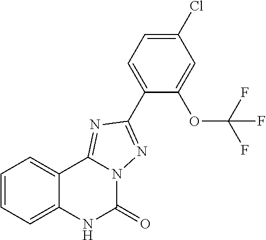 [SMILES: O=c1[nH]c2ccccc2c2nc(-c3ccc(Cl)cc3OC(F)(F)F)nn12] |
| | 2-[4-chloro-2-(trifluoromethoxy)phenyl][1,2,4]triazolo[1,5-c]quinazolin- |
| | 5(6H)-one |
| | LC-MS (Method 1): Rₜ = 1.31 min; MS (ESIpos): m/z = 381 [m + H]⁺ |
| | ¹H-NMR (400 MHz, DMSO-d₆): δ [ppm] = 7.40 − 7.48 (m, 2H), 7.70 − 7.78 (m, |
| | 3H), 8.19 (dd, 1H), 8.33 (d, 1H), 12.43 (br s, 1H). |
| Intermediate 267 | 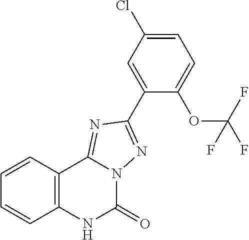 [SMILES: O=c1[nH]c2ccccc2c2nc(-c3cc(Cl)ccc3OC(F)(F)F)nn12] |
| | 2-[5-chloro-2-(trifluoromethoxy)phenyl][1,2,4]triazolo[1,5-c]quinazolin- |
| | 5(6H)-one |
| | LC-MS (Method 2): Rₜ = 0.84 min; MS (ESIpos): m/z = 381 [m + H]⁺ |
| | ¹H-NMR (400 MHz, DMSO-d₆): δ [ppm] = 7.41-7.48 (m, 2H), 7.66 (dd, 1H), |
| | 7.73 (ddd, 1H), 7.78 (dd, 1H), 8.22 (dd, 1H), 8.28 (d, 1H), 12.46 (br s, 1H). |
| Intermediate 268 | 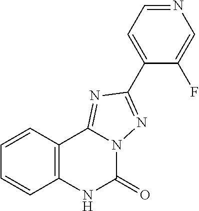 [SMILES: O=c1[nH]c2ccccc2c2nc(-c3ccncc3F)nn12] |
| | 2-(3-fluoropyridin-4-yl)[1,2,4]triazolo[1,5-c]quinazolin-5(6H)-one |
| | LC-MS (Method 2): Rₜ = 0.53 min; MS (ESIneg): m/z = 280 [m − H]⁻ |
| | ¹H-NMR (400 MHz, DMSO-d₆): δ [ppm] = 7.41 − 7.45 (m, 1H), 7.47 (d, 1H), |
| | 7.74 (ddd, 1H), 8.20 (dd, 1H), 8.23 (dd, 1H), 8.65 (d, 1H), 8.83 (d, 1H), 12.47 |
| | (br s, 1H). |
| Intermediate 269 | 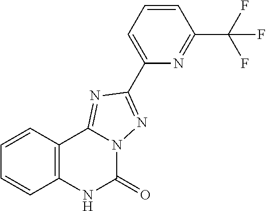 [SMILES: O=c1[nH]c2ccccc2c2nc(-c3cccc(C(F)(F)F)n3)nn12] |
| | 2-[6-(trifluoromethyl)pyridin-2-yl][1,2,4]triazolo[1,5-c]quinazolin-5(6H)- |
| | one |
| | LC-MS (Method 2): Rₜ = 0.66 min; MS (ESIpos): m/z = 332 [m + H]⁺ |
| | ¹H-NMR (400 MHz, DMSO-d₆): δ [ppm] = 7.42 − 7.46 (m, 1H), 7.48 (d, 1H), |
| | 7.75 (ddd, 1H), 8.09 (dd, 1H), 8.29 (dd, 1H), 8.34 (t, 1H), 8.58 (d, 1H), 12.45 (br s, 1H). |
| Intermediate 270 | 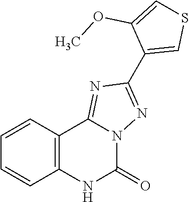 [SMILES: COc1cscc1-c1nc2c3ccccc3[nH]c(=O)n2n1] |
| | 2-(4-methoxythiophen-3-yl)[1,2,4]triazolo[1,5-c]quinazolin-5(6H)-one |
| | LC-MS (Method 2): Rₜ = 0.62 min; MS (ESIpos): m/z = 299 [m + H]⁺ |
| | ¹H-NMR (400 MHz, DMSO-d₆): δ [ppm] = 3.88 (s, 3H), 6.81 (d, 1H), 7.40 (t, |
| | 1H), 7.44 (d, 1H), 7.68 − 7.73 (m, 1H), 8.16 − 8.21 (m, 2H), 12.29 (br s, 1H). |
| Intermediate 271 | 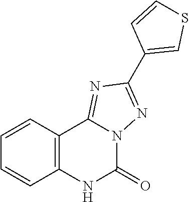 [SMILES: O=c1[nH]c2ccccc2c2nc(-c3ccsc3)nn12] |
| | 2-(thiophen-3-yl)[1,2,4]triazolo[1,5-c]quinazolin-5(6H)-one |
| | LC-MS (Method 2): Rₜ = 0.60 min; MS (ESIpos): m/z = 269 [m + H]⁺ |
| | ¹H-NMR (400 MHz, DMSO-d₆): δ [ppm] = 7.39 − 7.43 (m, 1H), 7.45 (d, 1H), |
| | 7.69 − 7.77 (m, 3H), 8.21 (dd, 1H), 8.32 (dd, 1H), 12.31 (br s, 1H). |
| Intermediate 272 | 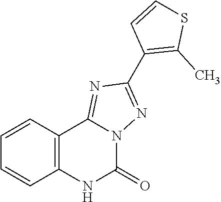 [SMILES: Cc1sccc1-c1nc2c3ccccc3[nH]c(=O)n2n1] |
| | 2-(2-methylthiophen-3-yl)[1,2,4]triazolo[1,5-c]quinazolin-5(6H)-one |
| | LC-MS (Method 2): Rₜ = 0.71 min; MS (ESIpos): m/z = 283 [m + H]⁺ |
| | ¹H-NMR (400 MHz, DMSO-d₆): δ [ppm] = 2.89 (s, 3H), 7.38 − 7.47 (m, 3H), |
| | 7.63 (d, 1H), 7.71 (ddd, 1H), 8.21 (dd, 1H), 12.33 (br s, 1H). |
| Intermediate 273 | 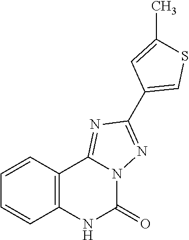 [SMILES: Cc1cc(-c2nc3c4ccccc4[nH]c(=O)n3n2)cs1] |
| | 2-(5-methylthiophen-3-yl)[1,2,4]triazolo[1,5-c]quinazolin-5(6H)-one |
| | LC-MS (Method 2): Rₜ = 0.69 min; MS (ESIpos): m/z = 283 [m + H]⁺ |
| | ¹H-NMR (400 MHz, DMSO-d₆): δ [ppm] = 2.54 (d, 3H), 7.38 − 7.47 (m, 3H), |
| | 7.71 (ddd, 1H), 8.05 (d, 1H), 8.19 (dd, 1H), 12.30 (br s, 1H). |

### Intermediate 274

### 2-(Imidazo[1,2-a]pyridin-7-yl)[1,2,4]triazolo[1,5-c]quinazolin-5(6H)-one

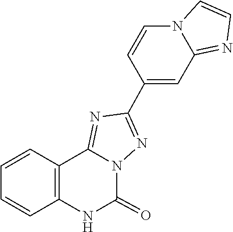 [SMILES: O=c1[nH]c2ccccc2c2nc(-c3ccn4ccnc4c3)nn12]

Methyl (2-cyanophenyl)carbamate (700 mg, 3.97 mmol) and imidazo[1,2-a]pyridine-7-carbohydrazide (700 mg, 3.97 mmol) were suspended in DMF (20 mL). It was stirred at 120° C. for 24 h. Imidazo[1,2-a]pyridine-7-carbohydrazide (100 mg, 0.57 mmol) was added and it was stirred at 120° C. for 24 h. The reaction mixture was allowed to cool down to rt and poured into water (100 mL). The precipitate was filtered off, washed four times with water and dried under vacuum at 50° C. to yield 1.02 g of a crude product. 100 mg of the crude product in DMF (2.5 mL) was stirred at 120° C. over the weekend. The reaction mixture was allowed to cool down to rt and poured into water. The precipitate was filtered off, washed three times with water and dried at 50° C. under vacuum affording 88 mg of a crude product. All two crude products were combined and stirred in DMF (20 mL) at 130° C. for 120 h. The reaction mixture was allowed to reach rt and poured into water. The precipitate was filtered off, washed three times with water, dried at 50° C. under vacuum affording 919 mg of the title compound which was used without further purification in the next step.

LC-MS (Method 2): Rₜ=0.56 min; MS (ESIpos): m/z=303 [m+H]⁺

¹H-NMR (400 MHz, DMSO-d₆): δ [ppm]=7.42-7.49 (m, 2H), 7.66 (dd, 1H), 7.70-7.77 (m, 2H), 8.11 (s, 1H), 8.27 (dd, 1H), 8.34 (s, 1H), 8.72 (d, 1H), 12.39 (br s, 1H).

### Intermediate 275

### Methyl 2-(4-methoxyphenyl)-5-oxo-5,6-dihydro[1,2,4]triazolo[1,5-c]quinazoline-10-carboxylate

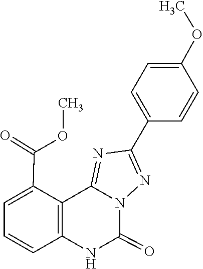 [SMILES: COC(=O)c1cccc2[nH]c(=O)n3nc(-c4ccc(OC)cc4)nc3c12]

10-Bromo-2-(4-methoxyphenyl)[1,2,4]triazolo[1,5-c]quinazolin-5(6H)-one (396 mg, 1.07 mmol) was suspended in methanol/THF (13.2 mL, 10:1) in an autoclave (50 mL). Triethylamine (300 μL, 2.1 mmol) and 1,1′-bis(diphenylphosphino)ferrocene-palladium(II)dichloride dichloromethane complex (90 mg, 110 μmol) were added. The reaction mixture was purged three times with carbon monoxide at rt. Then, the autoclave was filled with carbon monoxide up to 12.7 bar and it was stirred for 30 min at rt. As the pressure was constant at 12.6 bar the carbon monoxide was released and the autoclave was evacuated under vacuum. The autoclave was filled with carbon monoxide up to 14.2 bar at 20° C. internal temperature. The reaction mixture was stirred for 24 h at 100° C. internal temperature. The reaction mixture was allowed to cool down to rt and the carbon monoxide was removed. The reaction mixture was concentrated and digested in ethyl acetate/dichloromethane. The insoluble residue was filtered off, washed with ethyl acetate and a few drops of dichloromethane, and the filtrate was concentrated under reduce pressure to yield 326 mg (87%) of the title product.

LC-MS (Method 2): Rₜ=0.67 min; MS (ESIpos): m/z=351 [M+H]⁺

¹H-NMR (400 MHz, DMSO-d₆) δ [ppm]=3.85 (s, 3H), 4.01 (s, 3H), 7.11-7.17 (m, 2H), 7.43 (dd, 1H), 7.54 (dd, 1H), 7.76 (dd, 1H), 8.08-8.14 (m, 2H).

### Intermediate 276

### 5,7-Dichloro-2-(pyridin-4-yl)[1,2,4]triazolo[1,5-c]quinazoline

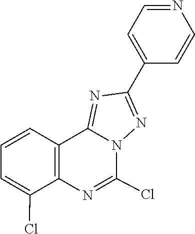 [SMILES: Clc1cccc2c1nc(Cl)n1nc(-c3ccncc3)nc21]

7-Chloro-2-(pyridin-4-yl)[1,2,4]triazolo[1,5-c]quinazolin-5(6H)-one (482 mg, 1.62 mmol) was stirred in POCl₃ (5.0 mL, 54 mmol) and N,N-diisopropylethylamine (2.8 mL, 16 mmol) overnight at 110° C. The mixture was poured into ice, and the solid was filtered, washed with water and dried under reduced pressure at 60° C. to give the title compound 530 mg (39% purity, 40% yield) that was used without further purification.

LC-MS (method 2): Rₜ=1.19 min; MS (ESIpos): m/z=316 [M+H]⁺

The following intermediates were prepared analogously to intermediate 275:

| | Structure |
|---|---|
| | IUPAC-Name |
| | LC-MS (method): Retention time; Mass found |
| Intermediate | ¹H-NMR |
| Intermediate 277 | 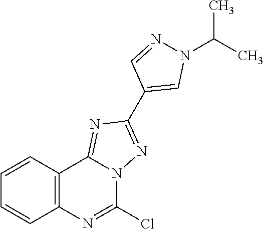 [SMILES: CC(C)n1cc(-c2nc3c4ccccc4nc(Cl)n3n2)cn1] |
| | 5-chloro-2-[1-(propan-2-yl)-1H-pyrazol-4-yl][1,2,4]triazolo[1,5- |
| | c]quinazoline |
| | LC-MS (Method 2): Rₜ = 1.15 min; MS (ESIpos): m/z = 313 [M + H]⁺ |
| | ¹H-NMR (400 MHz, DMSO-d₆): δ [ppm] = 1.50 (d, 6H), 4.64 (spt, 1H), 7.84 |
| | (ddd, 1H), 7.93 − 7.99 (m, 1H), 8.00 − 8.04 (m, 1H), 8.11 (s, 1H), 8.45 (dd, |
| | 1H), 8.57 (s, 1H). |
| Intermediate 278 | 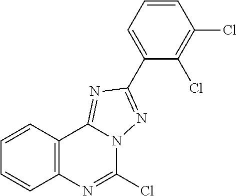 [SMILES: Clc1cccc(-c2nc3c4ccccc4nc(Cl)n3n2)c1Cl] |
| | 5-chloro-2-(2,3-dichlorophenyl)[1,2,4]triazolo[1,5-c]quinazoline |
| | LC-MS (Method 2): Rₜ = 1.48 min; MS (ESIpos): m/z = 349 [M + H]⁺ |
| Intermediate 279 | 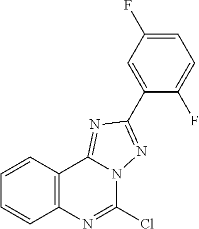 [SMILES: Fc1ccc(F)c(-c2nc3c4ccccc4nc(Cl)n3n2)c1] |
| | 5-chloro-2-(2,5-difluorophenyl)[1,2,4]triazolo[1,5-c]quinazoline |
| | LC-MS (Method 2): Rₜ = 1.36 min; MS (ESIpos): m/z = 317 [M + H]⁺ |
| Intermediate 280 | 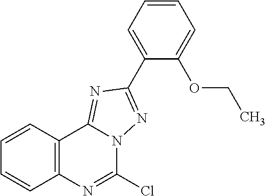 [SMILES: CCOc1ccccc1-c1nc2c3ccccc3nc(Cl)n2n1] |
| | 5-chloro-2-(2-ethoxyphenyl)[1,2,4]triazolo[1,5-c]quinazoline |
| | LC-MS (Method 2): Rₜ = 1.35 min; MS (ESIpos): m/z = 325 [M + H]⁺ |
| | ¹H-NMR (400 MHz, DMSO-d₆): δ [ppm] = 1.41 (t, 3H), 4.20 (q, 2H), 7.14 (td, |
| | 1H), 7.25 (d, 1H), 7.53 (ddd, 1H), 7.86 (ddd, 1H), 7.97 (td, 1H), 8.02 − 8.09 |
| | (m, 2H), 8.50 (dd, 1H). |
| Intermediate 281 | 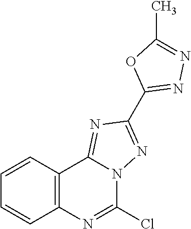 [SMILES: Cc1nnc(-c2nc3c4ccccc4nc(Cl)n3n2)o1] |
| | 5-chloro-2-(5-methyl-1,3,4-oxadiazol-2-yl)[1,2,4]triazolo[1,5- |
| | c]quinazoline |
| | LC-MS (Method 2): Rₜ = 0.94 min; MS (ESIpos): m/z = 287 [M + H]⁺ |
| Intermediate 282 | 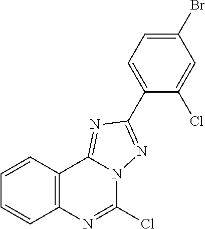 [SMILES: Clc1cc(Br)ccc1-c1nc2c3ccccc3nc(Cl)n2n1] |
| | 2-(4-bromo-2-chlorophenyl)-5-chloro[1,2,4]triazolo[1,5-c]quinazoline |
| | min; |
| | LC-MS (Method 2): Rₜ = 1.56 MS (ESIpos): m/z = 331 [M + H]⁺ |
| | ¹H-NMR (400 MHz, DMSO-d₆): δ [ppm] = 7.81 (dd, 1H), 7.88 (ddd, 1H), 7.99 − |
| | 8.03 (m, 2H), 8.05 − 8.08 (m, 1H), 8.10 (d, 1H), 8.51 (dd, 1H). |
| Intermediate 283 | 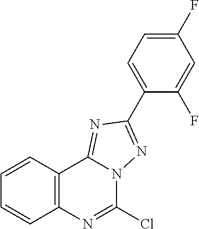 [SMILES: Fc1ccc(-c2nc3c4ccccc4nc(Cl)n3n2)c(F)c1] |
| | 5-chloro-2-(2,4-difluorophenyl)[1,2,4]triazolo[1,5-c]quinazoline |
| | LC-MS (Method 2): Rₜ = 1.35 min; MS (ESIpos): m/z = 317 [M + H]⁺ |
| | ¹H-NMR (400 MHz, DMSO-d₆): δ [ppm] = 7.32 − 7.39 (m, 1H), 7.54 (ddd, 1H), |
| | 7.87 (ddd, 1H), 7.96 − 8.02 (m, 1H), 8.03 − 8.08 (m, 1H), 8.35 (td, 1H), 8.51 |
| | (dd, 1H). |
| Intermediate 284 | 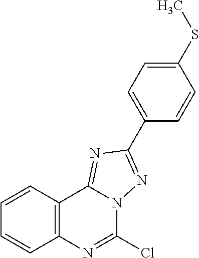 [SMILES: CSc1ccc(-c2nc3c4ccccc4nc(Cl)n3n2)cc1] |
| | 5-chloro-2-[4-(methylsulfanyl)phenyl][1,2,4]triazolo[1,5-c]quinazoline |
| | LC-MS (Method 2): Rₜ = 1.45 min; MS (ESIpos): m/z = 327 [M + H]⁺ |
| Intermediate 285 | 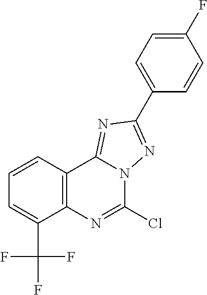 [SMILES: Fc1ccc(-c2nc3c4cccc(C(F)(F)F)c4nc(Cl)n3n2)cc1] |
| | 5-chloro-2-(4-fluorophenyl)-7-(trifluoromethyl)[1,2,4]triazolo[1,5- |
| | c]quinazoline |
| | LC-MS (Method 2): Rₜ = 1.49 min; MS (ESIpos): m/z = 367 [M + H]⁺ |
| Intermediate 286 | 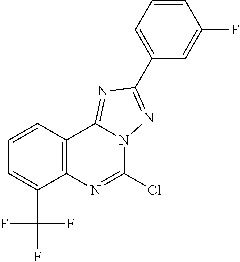 [SMILES: Fc1cccc(-c2nc3c4cccc(C(F)(F)F)c4nc(Cl)n3n2)c1] |
| | 5-chloro-2-(3-fluorophenyl)-7-(trifluoromethyl)[1,2,4]triazolo[1,5- |
| | c]quinazoline |
| | LC-MS (Method 2): Rₜ = 1.50 min; MS (ESIpos): m/z = 367 [M + H]⁺ |
| Intermediate 287 | 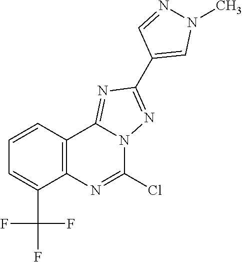 [SMILES: Cn1cc(-c2nc3c4cccc(C(F)(F)F)c4nc(Cl)n3n2)cn1] |
| | 5-chloro-2-(1-methyl-1H-pyrazol-4-yl)-7- |
| | (trifluoromethyl)[1,2,4]triazolo[1,5-c]quinazoline |
| | LC-MS (Method 2): Rₜ = 1.15 min; MS (ESIpos): m/z = 353 [M + H]⁺ |
| Intermediate 288 | 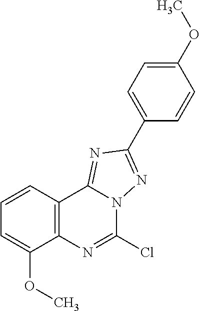 [SMILES: COc1ccc(-c2nc3c4cccc(OC)c4nc(Cl)n3n2)cc1] |
| | 5-chloro-7-methoxy-2-(4-methoxyphenyl)[1,2,4]triazolo[1,5- |
| | c]quinazoline |
| | LC-MS (Method 2): Rₜ = 1.27 min; MS (ESIpos): m/z = 341 [M + H]⁺ |
| | ¹H-NMR (400 MHz, DMSO-d₆): δ [ppm] = 3.86 (s, 3H), 4.02 (s, 3H), 7.09 − |
| | 7.21 (m, 2H), 7.51 (dd, 1H), 7.78 (t, 1H), 8.02 (dd, 1H), 8.15 − 8.25 (m, 2H). |
| Intermediate 289 | 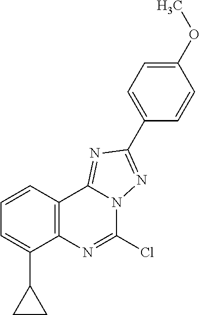 [SMILES: COc1ccc(-c2nc3c4cccc(C5CC5)c4nc(Cl)n3n2)cc1] |
| | 5-chloro-7-cyclopropyl-2-(4-methoxyphenyl)[1,2,4]triazolo[1,5- |
| | c]quinazoline |
| | LC-MS (Method 2): Rₜ = 1.56 min; MS (ESIpos): m/z = 351 [M + H]⁺ |
| | ¹H-NMR (400 MHz, DMSO-d₆): δ [ppm] = 0.87 − 0.93 (m, 2H), 1.15 − 1.21 (m, |
| | 2H), 2.98 (tt, 1H), 3.86 (s, 3H), 7.12 − 7.17 (m, 2H), 7.40 (dd, 1H), 7.72 (t, |
| | 1H), 8.20 − 8.25 (m, 2H), 8.27 (dd, 1H). |
| Intermediate 290 | 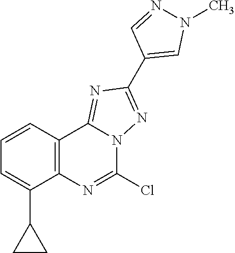 [SMILES: Cn1cc(-c2nc3c4cccc(C5CC5)c4nc(Cl)n3n2)cn1] |
| | 5-chloro-7-cyclopropyl-2-(1-methyl-1H-pyrazol-4-yl)[1,2,4]triazolo[1,5- |
| | c]quinazoline |
| | LC-MS (Method 2): Rₜ = 1.22 MS min; (ESIpos): m/z = 325 [M + H]⁺ |
| | ¹H-NMR (400 MHz, DMSO-d₆): δ [ppm] = 0.87 − 0.92 (m, 2H), 1.15 − 1.21 (m, |
| | 2H), 2.98 (tt, 1H), 3.96 (s, 3H), 7.39 (dd, 1H), 7.71 (t, 1H), 8.09 (d, 1H), 8.21 |
| | (dd, 1H), 8.53 (s, 1H). |
| Intermediate 291 | 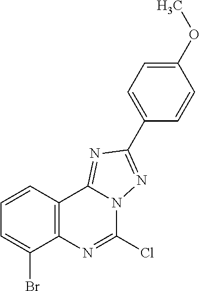 [SMILES: COc1ccc(-c2nc3c4cccc(Br)c4nc(Cl)n3n2)cc1] |
| | 7-bromo-5-chloro-2-(4-methoxyphenyl)[1,2,4]triazolo[1,5-c]quinazoline |
| | LC-MS (Method 2): Rₜ = 1.46 min; MS (ESIpos): m/z = 391 [M + H]⁺ |
| | ¹H-NMR (400 MHz, DMSO-d₆): δ [ppm] = 3.87 (s, 3H), 7.13 − 7.19 (m, 2H), |
| | 7.74 (t, 1H), 8.21-8.27 (m, 2H), 8.30 (dd, 1H), 8.52 (dd, 1H). |
| Intermediate 292 | 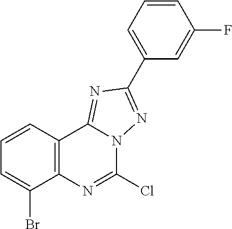 [SMILES: Fc1cccc(-c2nc3c4cccc(Br)c4nc(Cl)n3n2)c1] |
| | 7-bromo-5-chloro-2-(3-fluorophenyI)[1,2,4]triazolo[1,5-c]quinazoline |
| | LC-MS (Method 2): Rₜ = 1.50 min; MS (ESIpos): m/z = 377 [M + H]⁺ |
| | ¹H-NMR (400 MHz, DMSO-d₆): δ [ppm] = 7.41 − 7.49 (m, 2H), 7.76 (t, 1H), |
| | 8.29 − 8.39 (m, 3H), 8.53 (dd, 1H). |
| Intermediate 293 | 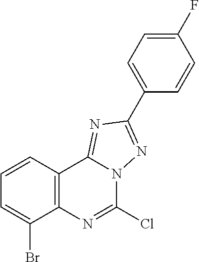 [SMILES: Fc1ccc(-c2nc3c4cccc(Br)c4nc(Cl)n3n2)cc1] |
| | 7-bromo-5-chloro-2-(4-fluorophenyl)[1,2,4]triazolo[1,5-c]quinazoline |
| | LC-MS (Method 2): Rₜ = 1.49 min; MS (ESIpos): m/z = 377 [M + H]⁺ |
| | ¹H-NMR (400 MHz, DMSO-d₆): δ [ppm] = 7.43 − 7.50 (m, 1H), 7.68 (td, 1H), |
| | 7.77 (t, 1H), 8.00 (ddd, 1H), 8.15 (dt, 1H), 8.32 (dd, 1H), 8.54 (dd, 1H). |
| Intermediate 294 | 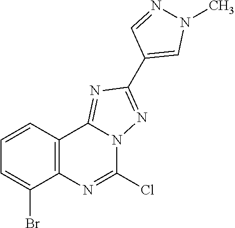 [SMILES: Cn1cc(-c2nc3c4cccc(Br)c4nc(Cl)n3n2)cn1] |
| | 7-bromo-5-chloro-2-(1-methyl-1H-pyrazol-4-yl)[1,2,4]triazolo[1,5- |
| | c]quinazoline |
| | LC-MS (Method 2): Rₜ = 1.11 min; MS (ESIpos): m/z = 363 [M + H]⁺ |
| Intermediate 295 | 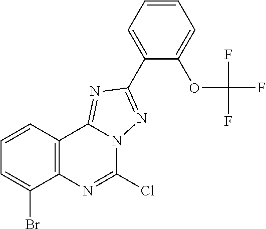 [SMILES: FC(F)(F)Oc1ccccc1-c1nc2c3cccc(Br)c3nc(Cl)n2n1] |
| | 7-bromo-5-chloro-2-[2-(trifluoromethoxy)phenyl][1,2,4]triazolo[1,5- |
| | c]quinazoline |
| | LC-MS (Method 2): Rₜ = 1.54 min; MS (ESIpos): m/z = 443 [M + H]⁺ |
| | 1H-NMR (400 MHz, DMSO-d₆): δ [ppm] = 7.62 − 7.71 (m, 2H), 7.72 − 7.81 (m, |
| | 2H), 8.33 (dd, 1H), 8.41 (dd, 1H), 8.51 (dd, 1H). |
| Intermediate 296 | 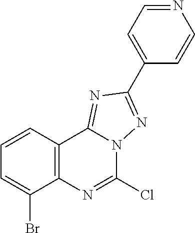 [SMILES: Clc1nc2c(Br)cccc2c2nc(-c3ccncc3)nn12] |
| | 7-bromo-5-chloro-2-(pyridin-4-yl)[1,2,4]triazolo[1,5-c]quinazoline |
| | LC-MS (Method 2): Rₜ = 1.22 min; MS (ESIpos): m/z = 360 [M + H]⁺ |
| Intermediate 297 | 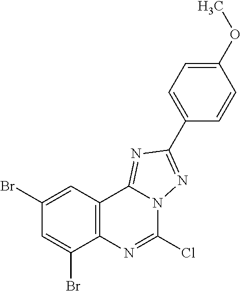 [SMILES: COc1ccc(-c2nc3c4cc(Br)cc(Br)c4nc(Cl)n3n2)cc1] |
| | 7,9-dibromo-5-chloro-2-(4-methoxyphenyl)0,2,4]triazolo[1,5- |
| | c]quinazoline |
| | LC-MS (Method 2.): Rₜ = 1.63 min; MS (ESIpos): m/z = 467 [M + H]⁺ |
| | ¹H-NMR (400 MHz, DMSO-d₆): δ [ppm] = 3.87 (s, 3H), 7.13 − 7.21 (m, 2H), |
| | 8.21 − 8.27 (m, 2H), 8.54 (d, 1H), 8.60 − 8.66 (m, 1H). |
| Intermediate 298 | 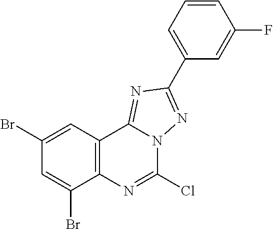 [SMILES: Fc1cccc(-c2nc3c4cc(Br)cc(Br)c4nc(Cl)n3n2)c1] |
| | 7,9-dibromo-5-chloro-2-(3-fluorophenyl)[1,2,4]triazolo[1,5-c]quinazoline |
| Intermediate 299 | 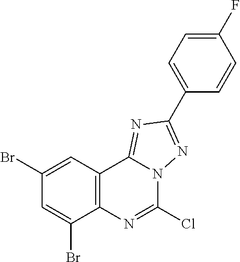 [SMILES: Fc1ccc(-c2nc3c4cc(Br)cc(Br)c4nc(Cl)n3n2)cc1] |
| | 7,9-dibromo-5-chloro-2-(4-fluorophenyl)[1,2,4]triazolo[1,5-c]quinazoline |
| | ¹H-NMR (400 MHz, DMSO-d₆): δ [ppm] = 7.42 − 7.52 (m, 2H), 8.32 − 8.40 (m, |
| | 2H), 8.56 (d, 1H), 8.65 (d, 1H). |
| Intermediate 300 | 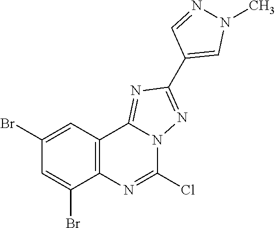 [SMILES: Cn1cc(-c2nc3c4cc(Br)cc(Br)c4nc(Cl)n3n2)cn1] |
| | 7,9-dibromo-5-chloro-2-(1-methyl-1H-pyrazol-4-yl)[1,2,4]triazolo[1,5- |
| | c]quinazoline |
| Intermediate 301 | 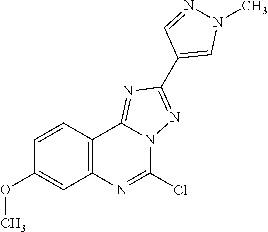 [SMILES: COc1ccc2c(c1)nc(Cl)n1nc(-c3cnn(C)c3)nc21] |
| | 5-chloro-8-methoxy-2-(1-methyl-1H-pyrazol-4-yl)[1,2,4]triazolo[1,5- |
| | c]quinazoline |
| | LC-MS (Method 2): Rₜ = 1.00 min; MS (ESIpos): m/z = 315 [M + H]⁺ |
| | ¹H NMR (400 MHz, DMSO-d₆) δ ppm 3.96 (s, 3H), 3.95 (s, 3 H)7.43 (dd, |
| | 1H), 7.49 (d, 1H), 8.04 − 8.09 (m, 1H), 8.33 (d, 1H), 8.51 (s, 1H). |
| Intermediate 302 | 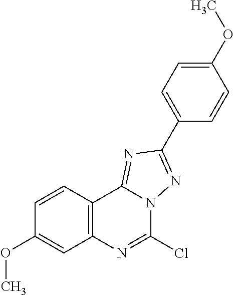 [SMILES: COc1ccc(-c2nc3c4ccc(OC)cc4nc(Cl)n3n2)cc1] |
| | 5-chloro-8-methoxy-2-(4-methoxyphenyl)[1,2,4]triazolo[1,5- |
| | c]quinazoline |
| | LC-MS (Method 2): Rₜ = 1.37 min; MS (ESIpos): m/z = 341 [M + H]⁺ |
| | ¹H-NMR (400 MHz, DMSO-d₆): δ [ppm] = 3.86 (s, 3H), 3.97 (s, 3H), 7.12 − |
| | 7.17 (m, 2H), 7.45 (dd, 1H), 7.51 (d, 1H), 8.19 − 8.24 (m, 2H), 8.39 (d, 1H). |
| Intermediate 303 | 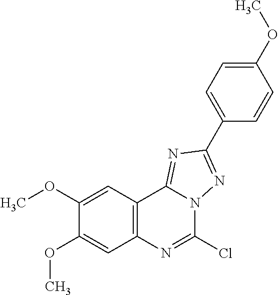 [SMILES: COc1ccc(-c2nc3c4cc(OC)c(OC)cc4nc(Cl)n3n2)cc1] |
| | 5-chloro-8,9-dimethoxy-2-(4-methoxyphenyl)[1,2,4]triazolo[1,5- |
| | c]quinazoline |
| | LC-MS (Method 2): Rₜ = 1.30 min; MS (ESIpos): m/z = 371 [M + H]⁺ |
| | ¹H-NMR (400 MHz, DMSO-d₆): δ [ppm] = 3.86 (s, 3H), 3.98 (s, 3H), 4.04 (s, |
| | 3H), 7.11 − 7.18 (m, 2H), 7.53 (s, 1H), 7.76 (s, 1H), 8.20 − 8.26 (m, 2H). |
| Intermediate 304 | 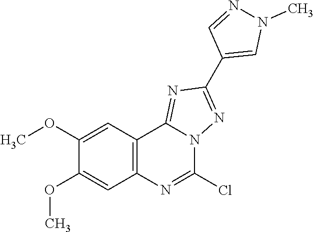 |
| | 5-chloro-8,9-dimethoxy-2-(1-methyl-1H-pyrazol-4-yl)[1,2,4]triazolo[1,5- |
| | c]quinazoline |
| | LC-MS (Method 2): Rₜ = 0.95 min; MS (ESIpos): m/z = 345 [M + H]⁺ |
| | ¹H NMR (400 MHz, DMSO-d₆) δ ppm 4.02 (s, 3 H), 3.97 (s, 3 H), 3.95 (s, 3 |
| | H)7.51 (s, 1H), 7.69 (s, 1H), 8.07 (d, 1H), 8.51 (s, 1H). |
| Intermediate 305 | 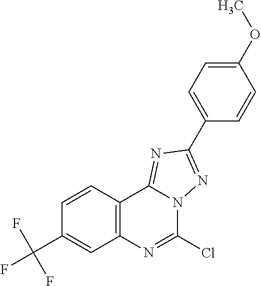 [SMILES: COc1ccc(-c2nc3c4ccc(C(F)(F)F)cc4nc(Cl)n3n2)cc1] |
| | 5-chloro-2-(4-methoxyphenyl)-8-(trifluoromethyl)[1,2,4]triazolo[1,5- |
| | c]quinazoline |
| | LC-MS (Method 2): Rₜ = 1.51 min; MS (ESIpos): m/z = 379 [M + H]⁺ |
| | ¹H-NMR (400 MHz, DMSO-d₆): δ [ppm] = 3.87 (s, 3H), 7.15 (d, 2H), 8.14 (dd, |
| | 1H), 8.21-8.26 (m, 2H), 8.41 (s, 1H), 8.70 (d, 1H). |
| Intermediate 306 | 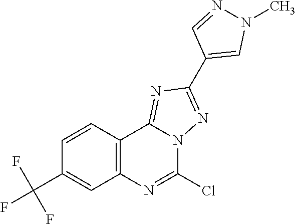 [SMILES: Cn1cc(-c2nc3c4ccc(C(F)(F)F)cc4nc(Cl)n3n2)cn1] |
| | 5-chloro-2-(1-methyl-1H-pyrazol-4-yl)-8- |
| | (trifluoromethyl)[1,2,4]triazolo[1,5-c]quinazoline |
| | LC-MS (Method 2): Rₜ = 1.20 min; MS (ESIpos): m/z = 353 [M + H]⁺ |
| | ¹H-NMR (400 MHz, DMSO-d₆): δ [ppm] = 3.96 (s, 3H), 7.72 (s, 1H), 8.12 − |
| | 8.15 (m, 1H), 8.41 (s, 1H), 8.57 (s, 1H), 8.65 (d, 1H). |
| Intermediate 307 | 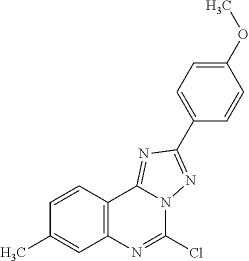 [SMILES: COc1ccc(-c2nc3c4ccc(C)cc4nc(Cl)n3n2)cc1] |
| | 5-chloro-2-(4-methoxyphenyl)-8-methyl[1,2,4]triazolo[1,5-c]quinazoline |
| | LC-MS (Method 2): Rₜ = 1.44 min; |
| | MS (ESIpos): m/z = 325 [M + H]⁺ |
| | ¹H-NMR (400 MHz, DMSO-d₆): δ [ppm] = 2.57 (s, 3H), 3.86 (s, 3H), 7.08 − |
| | 7.23 (m, 2H), 7.69 (dd, 1H), 7.84 (s, 1H), 8.18 − 8.28 (m, 2H), 8.39 (d, 1H). |
| Intermediate 308 | 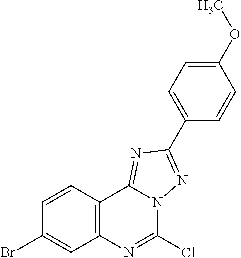 [SMILES: COc1ccc(-c2nc3c4ccc(Br)cc4nc(Cl)n3n2)cc1] |
| | 8-bromo-5-chloro-2-(4-methoxyphenyl)[1,2,4]triazolo[1,5-c]quinazoline |
| | LC-MS (Method 2): Rₜ = 1.54 min; MS (ESIpos): m/z = 389 [M + H]⁺ |
| | ¹H-NMR (400 MHz, DMSO-d₆): δ [ppm] = 3.87 (s, 3H), 7.14 − 7.17 (m, 2H), |
| | 8.01 (dd, 1H), 8.21 − 8.26 (m, 2H), 8.30 (d, 1H), 8.43 (d, 1H). |
| Intermediate 309 | 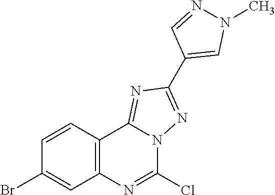 [SMILES: Cn1cc(-c2nc3c4ccc(Br)cc4nc(Cl)n3n2)cn1] |
| | 8-bromo-5-chloro-2-(1-methyl-1H-pyrazol-4-yl)[1,2,4]triazolo[1,5- |
| | c]quinazoline |
| | LC-MS (Method 2): Rt = 1.19 min; MS (ESIpos): m/z = 363 [M + H]⁺ |
| Intermediate 310 | 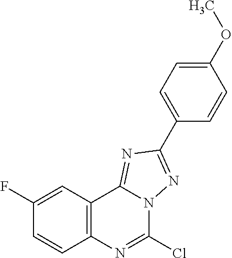 [SMILES: COc1ccc(-c2nc3c4cc(F)ccc4nc(Cl)n3n2)cc1] |
| | 5-chloro-9-fluoro-2-(4-methoxyphenyI)[1,2,4]triazolo[1,5-c]quinazoline |
| | LC-MS (Method 2): Rₜ = 1.40 min; MS (ESIpos): m/z = 329 [M + H]⁺ |
| | ¹H-NMR (400 MHz, DMSO-d₆): δ [ppm] = 3.86 (s, 3H), 7.13 − 7.19 (m, 2H), |
| | 7.86 (td, 1H), 8.12 (dd, 1H), 8.20 − 8.26 (m, 3H). |
| Intermediate 311 | 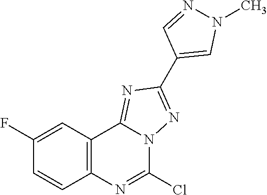 [SMILES: Cn1cc(-c2nc3c4cc(F)ccc4nc(Cl)n3n2)cn1] |
| | 5-chloro-9-fluoro-2-(1-methyl-1H-pyrazol-4-yl)[1,2,4]triazolo[1,5- |
| | c]quinazoline |
| | LC-MS (Method 2): Rₜ = 1.02 min; MS (ESIpos): m/z = 303 [M + H]⁺ |
| | ¹H-NMR (400 MHz, DMSO-d₆): δ [ppm] = 3.96 (s, 3H), 7.85 (td, 1H), 8.06 − |
| | 8.12 (m, 2H), 8.15 (dd, 1H), 8.52 (s, 1H). |
| Intermediate 312 | 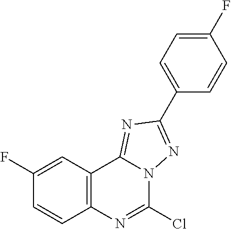 [SMILES: Fc1ccc(-c2nc3c4cc(F)ccc4nc(Cl)n3n2)cc1] |
| | 5-chloro-9-fluoro-2-(4-fluorophenyl)[1,2,4]triazolo[1,5-c]quinazoline |
| | LC-MS (Method 2): Rₜ = 1.43 min; MS (ESIpos): m/z = 317 [M + H]⁺ |
| Intermediate 313 | 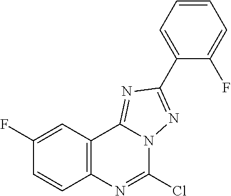 [SMILES: Fc1ccc2nc(Cl)n3nc(-c4ccccc4F)nc3c2c1] |
| | 5-chloro-9-fluoro-2-(2-fluorophenyl)[1,2,4]triazolo[1,5-c]quinazoline |
| | LC-MS (Method 2): Rₜ = 1.35 min; MS (ESIpos): m/z = 317 [M + H]⁺ |
| | ¹H-NMR (400 MHz, DMSO-d₆): δ [ppm] = 7.42 − 7.49 (m, 2H), 7.88 (td, 1H), |
| | 8.14 (dd, 1H), 8.26 (dd, 1H), 8.30 − 8.37 (m, 2H). |
| Intermediate 314 | 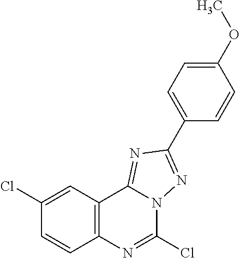 [SMILES: COc1ccc(-c2nc3c4cc(Cl)ccc4nc(Cl)n3n2)cc1] |
| | 5,9-dichloro-2-(4-methoxyphenyl)[1,2,4]triazolo[1,5-c]quinazoline |
| | LC-MS (Method 2): Rₜ = 1.50 min; MS (ESIpos): m/z = 345 [M + H]⁺ |
| | ¹H-NMR (400 MHz, DMSO-d₆): δ [ppm] = 3.87 (s, 3H), 7.10 − 7.22 (m, 2H), |
| | 7.97 − 8.01 (m, 1H), 8.04 − 8.08 (m, 1H), 8.16 − 8.27 (m, 2H), 8.47 (d, 1H). |
| Intermediate 315 | 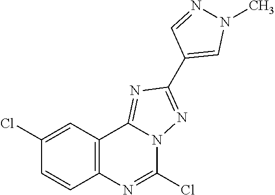 [SMILES: Cn1cc(-c2nc3c4cc(Cl)ccc4nc(Cl)n3n2)cn1] |
| | 5,9-dichloro-2-(1-methyl-1H-pyrazol-4-yl)[1,2,4]triazolo[1,5- |
| | c]quinazoline |
| | LC-MS (Method 2): Rₜ = 1.12 min; MS (ESIpos): m/z = 319 [M + H]⁺ |
| Intermediate 316 | 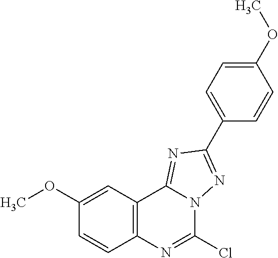 [SMILES: COc1ccc(-c2nc3c4cc(OC)ccc4nc(Cl)n3n2)cc1] |
| | 5-chloro-9-methoxy-2-(4-methoxyphenyl)[1,2,4]triazolo[1,5- |
| | c]quinazoline |
| | LC-MS (Method 2): Rₜ = 1.38 min; MS (ESIpos): m/z = 341 [M + H]⁺ |
| | ¹H-NMR (400 MHz, DMSO-d₆): δ [ppm] = 3.86 (s, 3H), 4.01 (s, 3H), 7.12 − |
| | 7.18 (m, 2H), 7.55 (dd, 1H), 7.80 (d, 1H), 7.95 (d, 1H), 8.20 − 8.28 (m, 2H). |
| Intermediate 317 | 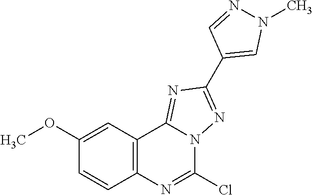 |
| | 5-chloro-9-methoxy-2-(1-methyl-1H-pyrazol-4-yl)[1,2,4]triazolo[1,5- |
| | c]quinazoline |
| | LC-MS (Method 2): Rₜ = 1.02 min; MS (ESIpos): m/z = 315 [M + H]⁺ |
| Intermediate 318 | 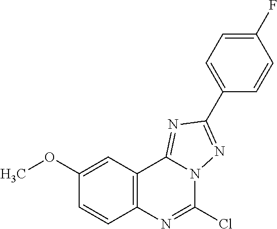 [SMILES: COc1ccc2nc(Cl)n3nc(-c4ccc(F)cc4)nc3c2c1] |
| | 5-chloro-2-(4-fluorophenyl)-9-methoxy[1,2,4]triazolo[1,5-c]quinazoline |
| | LC-MS (Method 2): Rₜ = 1.42 min; MS (ESIpos): m/z = 329 [M + H]⁺ |
| | ¹H-NMR (400 MHz, DMSO-d₆): δ [ppm] = 4.02 (s, 3H), 7.41 − 7.48 (m, 2H), |
| | 7.57 (dd, 1H), 7.83 (d, 1H), 7.98 (d, 1H), 8.32 − 8.38 (m, 2H). |
| Intermediate 319 | 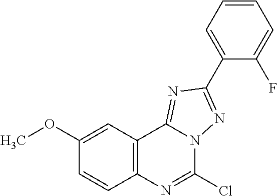 [SMILES: COc1ccc2nc(Cl)n3nc(-c4ccccc4F)nc3c2c1] |
| | 5-chloro-2-(2-fluorophenyl)-9-methoxy[1,2,4]triazolo[1,5-c]quinazoline |
| | LC-MS (Method 2): Rₜ = 1.34 min; MS (ESIpos): m/z = 329 [M + H]⁺ |
| | ¹H-NMR (400 MHz, DMSO-d₆): δ [ppm] = 4.02 (s, 3H), 7.42 − 7.51 (m, 2H), |
| | 7.57 (dd, 1H), 7.65 (dddd, 1H), 7.82 (d, 1H), 7.98 (d, 1H), 8.30 (td, 1H). |
| Intermediate 320 | 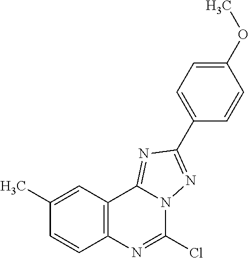 [SMILES: COc1ccc(-c2nc3c4cc(C)ccc4nc(Cl)n3n2)cc1] |
| | 5-chloro-2-(4-methoxyphenyl)-9-methyl[1,2,4]triazolo[1,5-c]quinazoline |
| | LC-MS (Method 2): Rₜ = 1.44 min; MS (ESIpos): m/z = 325 [M + H]⁺ |
| | ¹H-NMR (400 MHz, DMSO-d₆): δ [ppm] = 2.59 (s, 3H), 3.86 (s, 3H), 7.13 − |
| | 7.19 (m, 2H), 7.79 (dd, 1H), 7.92 (d, 1H), 8.19 − 8.25 (m, 2H), 8.29 − 8.32 (m, |
| | 1H). |
| Intermediate 321 | 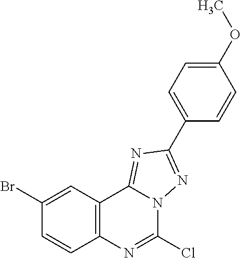 [SMILES: COc1ccc(-c2nc3c4cc(Br)ccc4nc(Cl)n3n2)cc1] |
| | 9-bromo-5-chloro-2-(4-methoxyphenyl)[1,2,4]triazolo[1,5-c]quinazoline |
| | LC-MS (Method 2): Rₜ = 1.52 min; MS (ESIpos): m/z = 389 [M + H]⁺ |
| | ¹H-NMR (400 MHz, DMSO-d₆): δ [ppm] = 3.86 (s, 3H), 7.14 − 7.19 (m, 2H), |
| | 7.97 (d, 1H), 8.11 (dd, 1H), 8.21-8.26 (m, 2H), 8.61 (d, 1H). |
| Intermediate 322 | 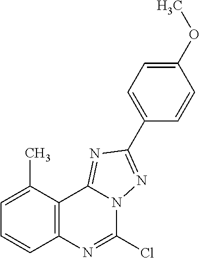 [SMILES: COc1ccc(-c2nc3c4c(C)cccc4nc(Cl)n3n2)cc1] |
| | 5-chloro-2-(4-methoxyphenyl)-10-methyl[1,2,4]triazolo[1,5- |
| | c]quinazoline |
| | LC-MS (Method 2): Rₜ = 1.54 min; MS (ESIpos): m/z = 325 [M + H]⁺ |
| | ¹H-NMR (500 MHz, DMSO-d₆): δ [ppm] = 3.10 (s, 3H), 3.86 (s, 3H), 7.16 (d, |
| | 2H), 7.66 − 7.70 (m, 1H), 7.81 − 7.87 (m, 2H), 8.22 − 8.27 (m, 2H). |
| Intermediate 323 | 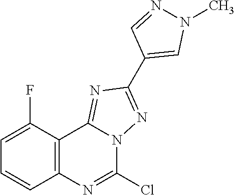 [SMILES: Cn1cc(-c2nc3c4c(F)cccc4nc(Cl)n3n2)cn1] |
| | 5-chloro-10-fluoro-2-(1-methyl-1H-pyrazol-4-yl)[1,2,4]triazolo[1,5- |
| | c]quinazoline |
| | LC-MS (Method 2): Rₜ = 0.95 min; MS (ESIpos): m/z = 303 [M + H]⁺ |
| Intermediate 324 | 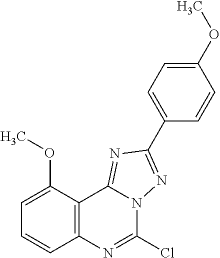 [SMILES: COc1ccc(-c2nc3c4c(OC)cccc4nc(Cl)n3n2)cc1] |
| | 5-chloro-10-methoxy-2-(4-methoxyphenyI)[1,2,4]triazolo[1,5- |
| | c]quinazoline |
| | LC-MS (Method 2): Rₜ = 1.26 min; MS (ESIpos): m/z = 341 [M + H]⁺ |
| | ¹H-NMR (400 MHz, DMSO-d₆): δ [ppm] = 3.86 (s, 3H), 4.11 (s, 3H), 7.14 − |
| | 7.18 (m, 2H), 7.41 (d, 1H), 7.57 (dd, 1H), 7.88 (t, 1H), 8.19 − 8.26 (m, 2H). |
| Intermediate 325 | 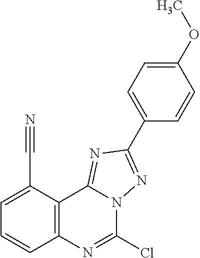 [SMILES: COc1ccc(-c2nc3c4c(C#N)cccc4nc(Cl)n3n2)cc1] |
| | 5-chloro-2-(4-methoxyphenyl)[1,2,4]triazolo[1,5-c]quinazoline-10- |
| | carbonitrile |
| | LC-MS (Method 2): Rt = 1.30 min; MS (ESIpos): m/z = 336 [M + H]⁺ |
| | ¹H-NMR (400 MHz, DMSO-d₆): δ [ppm] = 3.87 (s, 3H), 7.17 − 7.22 (m, 2H), |
| | 8.09 (dd, 1H), 8.22 − 8.28 (m, 2H), 8.35 (dd, 1H), 8.39 (dd, 1H). |
| Intermediate 326 | 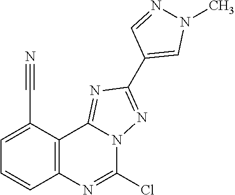 [SMILES: Cn1cc(-c2nc3c4c(C#N)cccc4nc(Cl)n3n2)cn1] |
| | 5-chloro-2-(1-methyl-1H-pyrazol-4-yl)[1,2,4]triazolo[1,5-c]quinazoline- |
| | 10-carbonitrile |
| | LC-MS (Method 2): Rₜ = 0.95 min; MS (ESIpos): m/z = 310 [M + H]⁺ |
| | ¹H-NMR (400 MHz, DMSO-d₆): δ [ppm] = 3.98 (s, 3H), 8.04 − 8.10 (m, 2H), |
| | 8.33 (dd, 1H), 8.37 (dd, 1H), 8.51 (s, 1H). |
| Intermediate 327 | 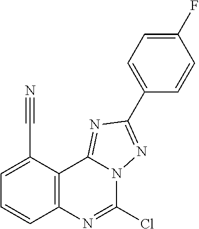 [SMILES: N#Cc1cccc2nc(Cl)n3nc(-c4ccc(F)cc4)nc3c12] |
| | 5-chloro-2-(4-fluorophenyl)[1,2,4]triazolo[1,5-c]quinazoline-10- |
| | carbonitrile |
| | LC-MS (Method 2): Rₜ = 1.32 min; MS (ESIpos): m/z = 324 [M + H]⁺ |
| | ¹H-NMR (400 MHz, DMSO-d₆): δ [ppm] = 7.46 − 7.53 (m, 2H), 8.11 (dd, 1H), |
| | 8.33 − 8.39 (m, 3H), 8.41 (dd, 1H). |
| Intermediate 328 | 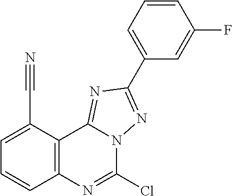 [SMILES: N#Cc1cccc2nc(Cl)n3nc(-c4cccc(F)c4)nc3c12] |
| | 5-chloro-2-(3-fluorophenyl)[1,2,4]triazolo[1,5-c]quinazoline-10- |
| | carbonitrile |
| | LC-MS (Method 2): Rₜ = 1.32 min; MS (ESIpos): m/z = 324 [M + H]⁺ |
| | ¹H-NMR (400 MHz, DMSO-d₆): δ [ppm] = 7.46 − 7.53 (m, 1H), 7.72 (td, 1H), |
| | 7.99 (ddd, 1H), 8.12 (dd, 1H), 8.16 (dt, 1H), 8.37 (dd, 1H), 8.42 (dd, 1H). |

### Intermediate 329

### 5-Chloro-2-[2-(trifluoromethoxy)phenyl][1,2,4]triazolo[1,5-c]quinazoline

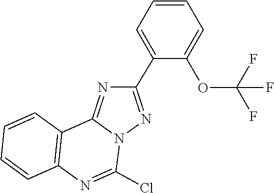 [SMILES: FC(F)(F)Oc1ccccc1-c1nc2c3ccccc3nc(Cl)n2n1]

2-[2-(Trifluoromethoxy)phenyl][1,2,4]triazolo[1,5-c]quinazolin-5(6H)-one (600 mg, 1.73 mmol) was suspended in phosphoric trichloride (4.8 mL, 51.13 mmol). N,N-Diisopropylethylamine (3.0 mL, 17 mmol) was added and it was stirred at 110° C. for 4.5 h. The reaction mixture was allowed to cool down to rt, poured into ice/water and stirred for 30 minutes. The precipitate was filtered, washed three times with water and dried under vacuum at 50° C. overnight yielding 585 mg (93%) of the title compound which was used without further purification in the next step.

LC-MS (Method 2): Rₜ=1.48 min; MS (ESIpos): m/z=365 [m+H]⁺

¹H-NMR (400 MHz, DMSO-d₆): δ [ppm]=7.61-7.69 (m, 2H), 7.72-7.77 (m, 1H), 7.89 (ddd, 1H), 8.00 (ddd, 1H), 8.05-8.08 (m, 1H), 8.40 (dd, 1H), 8.50 (dd, 1H).

The following intermediates were prepared analogously to intermediate 328:

| | Structure |
|---|---|
| | IUPAC-Name |
| | LC-MS (method): Retention time; Mass found |
| Intermediate | ¹H-NMR |
| Intermediate 330 | 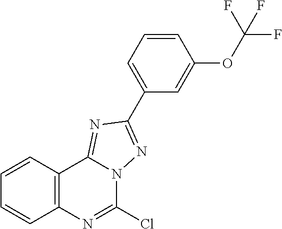 [SMILES: FC(F)(F)Oc1cccc(-c2nc3c4ccccc4nc(Cl)n3n2)c1] |
| | 5-chloro-2-[3-(trifluoromethoxy)phenyl] |
| | [1,2,4]triazolo[1,5-c]quinazoline |
| | LC-MS (Method 2): Rₜ = 1.55 min; |
| | MS (ESIpos): m/z = 365 [m + H]⁺ |
| | ¹H-NMR (400 MHz, DMSO-d₆): δ [ppm] = |
| | 7.61-7.65 (m, 1H), 7.78 (t, 1H), |
| | 7.88 (ddd, 1H), 8.00 (ddd, 1H), 8.05-8.08 |
| | (m, 1H), 8.14-8.17 (m, 1H), 8.32- |
| | 8.35 (m, 1H), 8.55 (dd, 1H). |
| Intermediate 331 | 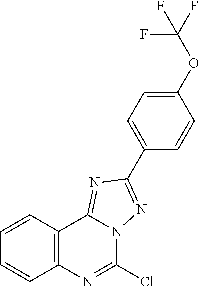 [SMILES: FC(F)(F)Oc1ccc(-c2nc3c4ccccc4nc(Cl)n3n2)cc1] |
| | 5-chloro-2-[4-(trifluoromethoxy) |
| | phenyl][1,2,4]triazolo[1,5-c] |
| | quinazoline |
| | LC-MS (Method 2): Rₜ = |
| | 1.55 min; MS (ESIpos): |
| | m/z = 365 [m + H]⁺ |
| | ¹H-NMR (400 MHz, |
| | DMSO-d₆): |
| | δ [ppm] = 7.58-7.63 |
| | (m, 2H), 7.88 (ddd, 1H), |
| | 7.97-8.02 (m, 1H), |
| | 8.04-8.07 (m, 1H), |
| | 8.42 (d, 2H), 8.53 (ddd, 1H). |
| Intermediate 332 | 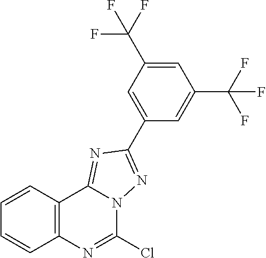 [SMILES: FC(F)(F)c1cc(-c2nc3c4ccccc4nc(Cl)n3n2)cc(C(F)(F)F)c1] |
| | 2-[3,5-bis(trifluoromethyl)phenyl]- |
| | 5-chloro[1,2,4]triazolo[1,5- |
| | c]quinazoline |
| | LC-MS (Method 2): Rₜ = 1.63 min; |
| | MS (ESIpos): m/z = 417 [m + H]⁺ |
| | ¹H-NMR (400 MHz, DMSO-d₆): |
| | δ [ppm] = 7.89 (ddd, 1H), |
| | 7.98-8.03 (m, 1H), |
| | 8.04-8.07 (m, 1H), 8.41 (s, 1H), |
| | 8.58 (dd, 1H), 8.75 (s, 2H). |
| Intermediate 333 | 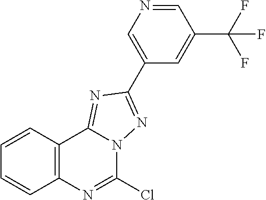 [SMILES: FC(F)(F)c1cncc(-c2nc3c4ccccc4nc(Cl)n3n2)c1] |
| | 5-chloro-2-[5-(trifluoromethyl) |
| | pyridin-3-yl] |
| | [1,2,4]triazolo[1,5- |
| | c]quinazoline |
| | LC-MS (Method 2): Rₜ = 1.38 min; |
| | MS (ESIpos): m/z = 350 [m + H]⁺ |
| | ¹H-NMR (400 MHz, DMSO-d₆): δ [ppm] = |
| | 7.90 (ddd, 1H), 8.02 (ddd, 1H), 8.07- |
| | 8.10 (m, 1H), 8.58 (dd, 1H), 8.83 (t, 1H), |
| | 9.23 (d, 1H), 9.71 (d, 1H). |
| Intermediate 334 | 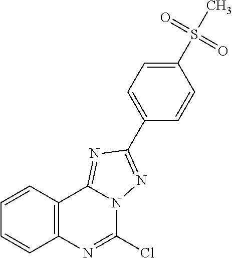 [SMILES: CS(=O)(=O)c1ccc(-c2nc3c4ccccc4nc(Cl)n3n2)cc1] |
| | 5-chloro-2-[4-(methanesulfonyl) |
| | phenyl] |
| | [1,2,4]triazolo[1,5-c]quinazoline |
| | LC-MS (Method 2): |
| | Rₜ = 1.16 min; MS (ESIpos): |
| | m/z = 359 [m + H]⁺ |
| | ¹H-NMR |
| | (400 MHz, DMSO-d₆): δ [ppm] = |
| | 3.32 (s, 3H), 7.87-7.92 (m, 1H), |
| | 8.01 (ddd, 1H), |
| | 8.05-8.09 (m, 1H), |
| | 8.15-8.19 (m, 2H), |
| | 8.53-8.57 (m, 3H). |
| Intermediate 335 | 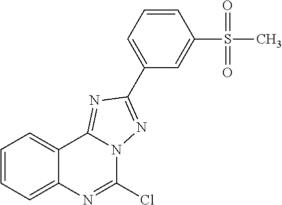 [SMILES: CS(=O)(=O)c1cccc(-c2nc3c4ccccc4nc(Cl)n3n2)c1] |
| | 5-chloro-2-[3-(methanesulfonyl)phenyl] |
| | [1,2,4]triazolo[1,5-c]quinazoline |
| | LC-MS (Method 1): Rₜ = 1.14 min; |
| | MS (ESIpos): m/z = 359 [m + H]⁺ |
| | ¹H-NMR (400 MHz, DMSO-d₆): δ [ppm] = |
| | 3.36 (s, 3H), 7.87-7.94 (m, 2H), |
| | 7.99-8.03 (m, 1H), 8.06-8.09 (m, 1H), 8.17 |
| | (ddd, 1H), 8.58 (dd, 1H), 8.63 |
| | (dt, 1H), 8.77 (t, 1H). |
| Intermediate 336 | 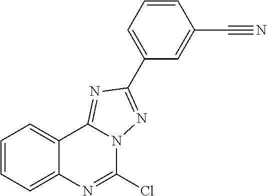 [SMILES: N#Cc1cccc(-c2nc3c4ccccc4nc(Cl)n3n2)c1] |
| | 3-(5-chloro[1,2,4]triazolo[1,5-c] |
| | quinazolin-2-yl)benzonitrile |
| | LC-MS (Method 1): Rₜ = 1.31 min; |
| | MS (ESIpos): m/z = 306 [m + H]⁺ |
| | ¹H-NMR (400 MHz, DMSO-d₆): δ [ppm] = |
| | 7.85 (t, 1H), 7.88-7.92 (m, 1H), |
| | 7.98-8.04 (m, 1H), 8.06-8.11 (m, 2H), |
| | 8.55 (dd, 1H), 8.59-8.64 (m, 2H). |
| Intermediate 337 | 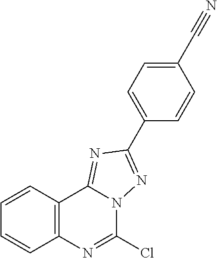 [SMILES: N#Cc1ccc(-c2nc3c4ccccc4nc(Cl)n3n2)cc1] |
| | 4-(5-chloro[1,2,4]triazolo[1,5-c] |
| | quinazolin-2-yl)benzonitrile |
| | LC-MS (Method 1): Rₜ = 1.33 min; |
| | MS (ESIpos): m/z = 306 [m + H]⁺ |
| | ¹H-NMR (400 MHz, DMSO-d₆): |
| | δ [ppm] = 7.90 (ddd, 1H), |
| | 7.99-8.03 (m, 1H), |
| | 8.06-8.11 (m, 3H), 8.45-8.49 |
| | (m, 2H), 8.55 (dd, 1H). |
| Intermediate 338 | 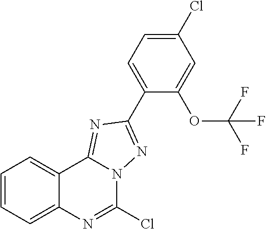 [SMILES: FC(F)(F)Oc1cc(Cl)ccc1-c1nc2c3ccccc3nc(Cl)n2n1] |
| | 5-chloro-2-[4-chloro-2-(trifluoromethoxy) |
| | phenyl][1,2,4]triazolo[1,5- |
| | c]quinazoline |
| | LC-MS (Method 1): Rₜ = 1.60 min; MS |
| | (ESIpos): m/z = 399 [m + H]⁺ |
| | ¹H-NMR (400 MHz, DMSO-d₆): δ [ppm] = |
| | 7.79 (dd, 1H), 7.80-7.82 (m, 1H), |
| | 7.89 (ddd, 1H), 7.98-8.03 (m, 1H), 8.05- |
| | 8.08 (m, 1H), 8.44 (d, 1H), 8.50 |
| | (dd, 1H). |
| Intermediate 339 | 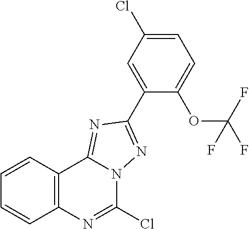 [SMILES: FC(F)(F)Oc1ccc(Cl)cc1-c1nc2c3ccccc3nc(Cl)n2n1] |
| | 5-chloro-2-[5-chloro-2-(trifluoromethoxy) |
| | phenyl][1,2,4]triazolo[1,5- |
| | c]quinazoline |
| | LC-MS (Method 2): Rₜ = 1.63 min; |
| | MS (ESIpos): m/z = 399 [m + H]⁺ |
| | ¹H-NMR (400 MHz, DMSO-d₆): |
| | δ [ppm] = |
| | 7.68-7.72 (m, 1H), 7.83 (dd, 1H), |
| | 7.89 (ddd, 1H), 7.98-8.03 (m, 1H), 8.05- |
| | 8.09 (m, 1H), 8.38 (d, 1H), 8.53 |
| | (dd, 1H). |
| Intermediate 340 | 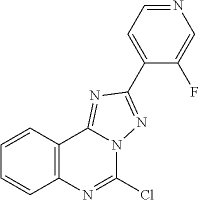 [SMILES: Fc1cnccc1-c1nc2c3ccccc3nc(Cl)n2n1] |
| | 5-chloro-2-(3-fluoropyridin-4-yl) |
| | [1,2,4]triazolo[1,5-c]quinazoline |
| | LC-MS (Method 2): |
| | Rₜ = 1.13 min; MS (ESIpos): |
| | m/z = 300 [m + H]⁺ |
| | ¹H-NMR (400 MHz, DMSO-d₆): |
| | δ [ppm] = 7.86-7.94 |
| | (m, 1H), 7.98-8.14 (m, |
| | 2H), 8.27 (br t, 1H), |
| | 8.54 (br d, 1H), 8.66- |
| | 8.74 (m, 1H), 8.87 (br s, 1H). |
| Intermediate 341 | 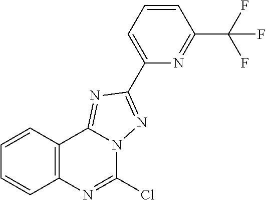 [SMILES: FC(F)(F)c1cccc(-c2nc3c4ccccc4nc(Cl)n3n2)n1] |
| | 5-chloro-2-[6-(trifluoromethyl)pyridin-2-yl] |
| | [1,2,4]triazolo[1,5- |
| | c]quinazoline |
| | LC-MS (Method 2): Rₜ = 1.28 min; MS |
| | (ESIpos): m/z = 350 [m + H]⁺ |
| | ¹H-NMR (400 MHz, DMSO-d₆): δ [ppm] = |
| | 7.91 (ddd, 1H), 8.00-8.04 (m, 1H), |
| | 8.07-8.11 (m, 1H), 8.14 (dd, 1H), 8.35-8.40 |
| | (m, 1H), 8.59 (dd, 1H), 8.67 |
| | (d, 1H). |
| Intermediate 342 | 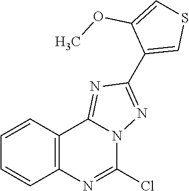 [SMILES: COc1cscc1-c1nc2c3ccccc3nc(Cl)n2n1] |
| | 5-chloro-2-(4- |
| | methoxythiophen-3-yl)[1,2,4] |
| | triazolo[1,5-c]quinazoline |
| | LC-MS (Method 2): |
| | Rₜ = 1.20 min; MS |
| | (ESIpos): m/z = 317 [m + H]⁺ |
| | ¹H-NMR (400 MHz, |
| | DMSO-d₆): δ [ppm] = 3.90 |
| | (s, 3H), 6.86 (d, 1H), 7.85 (ddd, |
| | 1H), 7.94-8.00 (m, 1H), 8.02- |
| | 8.05 (m, 1H), 8.30 |
| | (d, 1H), 8.48 (dd, 1H). |
| Intermediate 343 | 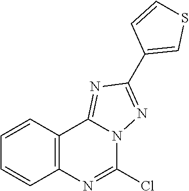 [SMILES: Clc1nc2ccccc2c2nc(-c3ccsc3)nn12] |
| | 5-chloro-2-(thiophen-3-yl) |
| | [1,2,4] |
| | triazolo[1,5-c]quinazoline |
| | LC-MS (Method 2): Rₜ = |
| | 1.29 min; MS (ESIpos): |
| | m/z = 287 [m + H]⁺ |
| | ¹H-NMR (400 MHz, DMSO- |
| | d₆): δ [ppm] = 7.77-7.82 |
| | (m, 2H), 7.84-7.88 (m, |
| | 1H), 7.95-8.01 (m, 1H), |
| | 8.02-8.06 (m, 1H), |
| | 8.44 (dd, 1H), 8.50 (dd, 1H). |
| Intermediate 344 | 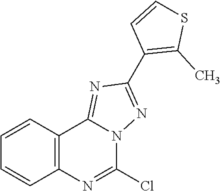 [SMILES: Cc1sccc1-c1nc2c3ccccc3nc(Cl)n2n1] |
| | 5-chloro-2-(2-methylthiophen-3-yl) |
| | [1,2,4]triazolo[1,5-c]quinazoline |
| | LC-MS (Method 2): Rₜ = 1.47 min; |
| | MS (ESIpos): m/z = 301 [m + H]⁺ |
| | ¹H-NMR (400 MHz, DMSO-d₆): |
| | δ [ppm] = |
| | 2.93 (s, 3H), 7.48 (d, 1H), 7.68 (d, |
| | 1H), 7.83-7.88 (m, 1H), 7.97 |
| | (ddd, 1H), 8.04 (d, 1H), |
| | 8.50 (dd, 1H). |
| Intermediate 345 | 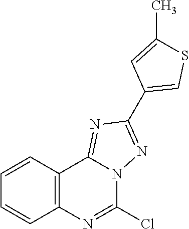 [SMILES: Cc1cc(-c2nc3c4ccccc4nc(Cl)n3n2)cs1] |
| | 5-chloro-2-(5-methylthiophen- |
| | 3-yl)[1,2,4] |
| | triazolo[1,5-c]quinazoline |
| | LC-MS (Method 2): |
| | Rₜ = 1.39 min; MS |
| | (ESIpos): m/z = 301 [m + H]⁺ |
| | ¹H-NMR (400 MHz, |
| | DMSO-d₆): |
| | δ [ppm] = 2.55 (d, 3H), |
| | 7.49-7.54 (m, 1H), 7.83-7.88 |
| | (m, 1H), 7.97 (ddd, 1H), |
| | 8.01-8.05 (m, 1H), 8.17 |
| | (d, 1H), 8.48 (dd, 1H). |

### Intermediate 346

### 5-Chloro-2-(imidazo[1,2-a]pyridin-7-yl)[1,2,4]triazolo[1,5-c]quinazoline

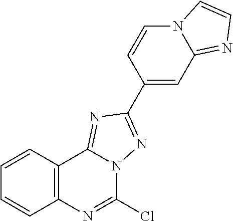 [SMILES: Clc1nc2ccccc2c2nc(-c3ccn4ccnc4c3)nn12]

2-(Imidazo[1,2-a]pyridin-7-yl)[1,2,4]triazolo[1,5-c]quinazolin-5(6H)-one (110 mg, 364 μmol) was suspended in phosphoric trichloride (1.0 mL, 10.7 mmol). N,N-Diisopropylethylamine (630 μL, 3.6 mmol) was added and it was stirred at 110° C. for 6 h. Phosphoric trichloride (1.0 mL, 10.7 mmol) was added and it was stirred at 110° C. for 6 h. The reaction mixture was allowed to cool down to rt, poured into ice/water and stirred for some minutes. The precipitate was filtered, washed three times with water and dried under vacuum at 50° C. overnight to give 89 mg of the title compound which was used without further purification in the next step.

LC-MS (Method 2): Rₜ=1.04 min; MS (ESIpos): m/z=321 [m+H]⁺

### Intermediate 347

### Methyl 5-chloro-2-(4-methoxyphenyl)[1,2,4]triazolo[1,5-c]quinazoline-10-carboxylate

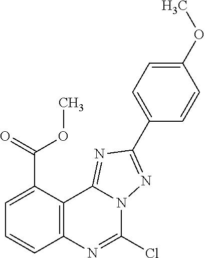 [SMILES: COC(=O)c1cccc2nc(Cl)n3nc(-c4ccc(OC)cc4)nc3c12]

Methyl 2-(4-methoxyphenyl)-5-oxo-5,6-dihydro[1,2,4]triazolo[1,5-c]quinazoline-10-carboxylate (20.0 mg, 57.1 μmol) was solubilised in POCl₃ (190 μl, 2.0 mmol), N,N-diisopropylethylamine (99 μl, 570 μmol) was added carefully and the mixture was stirred for 4 h at 110° C. The mixture was cooled to rt and poured into ice. The solid was filtered, washed with water and dried at 60° C. under reduced pressure to give 14.5 mg (70% purity, 48% yield) of the title compound that was used without further purification

LC-MS (method 2): Rₜ=1.34 min; MS (ESIpos): m/z=369 [M+H]⁺

### Intermediate 348

### Benzyl (6R)-6-{[7-chloro-2-(1-methyl-1H-pyrazol-4-yl)[1,2,4]triazolo[1,5-c]quinazolin-5-yl]amino}-5-oxo-1,4-diazepane-1-carboxylate

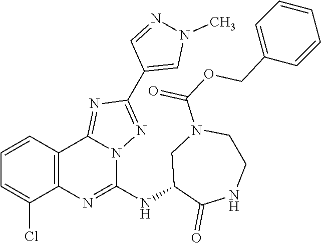 [SMILES: Cn1cc(-c2nc3c4cccc(Cl)c4nc(N[C@@H]4CN(C(=O)OCc5ccccc5)CCNC4=O)n3n2)cn1]

5,7-Dichloro-2-(1-methyl-1H-pyrazol-4-yl)[1,2,4]triazolo[1,5-c]quinazoline (75.0 mg, 235 μmol), benzyl (6R)-6-amino-5-oxo-1,4-diazepane-1-carboxylate hydrochloride (77.5 mg, 258 μmol) and N,N-diisopropylethylamine (120 μl, 700 μmol) were stirred in DMSO (1.6 mL) for 2 h at 60° C. The reaction was quenched with water and the solid was filtered, washed with water and dried under reduced pressure at 60° C. to give 100 mg (95% purity, 74% yield) of the title compound.

LC-MS (method 2): Rₜ=1.17 min; MS (ESIpos): m/z=546 [M+H]⁺

¹H-NMR (400 MHz, DMSO-d6): δ [ppm]=3.01-3.21 (m, 1H), 3.37-3.56 (m, 2H), 3.96 (s, 3H), 4.06-4.16 (m, 1H), 4.35-4.50 (m, 1H), 4.89-5.21 (m, 3H), 6.98-7.45 (m, 6H), 7.86 (br d, 1H), 8.07 (d, 2H), 8.22 (dd, 1H), 8.31-8.44 (m, 1H), 8.49 (s, 1H). (One proton is not visible.)

The following intermediates were prepared analogously to intermediate 347:

| | Structure |
|---|---|
| | IUPAC-Name |
| | LC-MS (method): Retention time; Mass found |
| Intermediate | ¹H-NMR |
| Intermediate 349 | 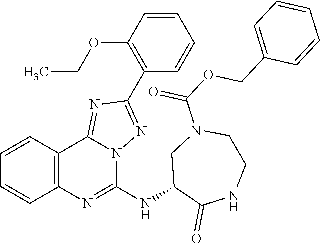 [SMILES: CCOc1ccccc1-c1nc2c3ccccc3nc(N[C@@H]3CN(C(=O)OCc4ccccc4)CCNC3=O)n2n1] |
| | benzyl (6R)-6-{[2-(2-ethoxyphenyl)[1,2,4] |
| | triazolo[1,5-c]quinazolin-5- |
| | yl]amino}-5-oxo-1,4-diazepane-1-carboxylate |
| | LC-MS (Method 2): Rₜ = 1.37 min; MS (ESIpos): |
| | m/z = 552 [M + H]⁺ |
| Intermediate 350 | 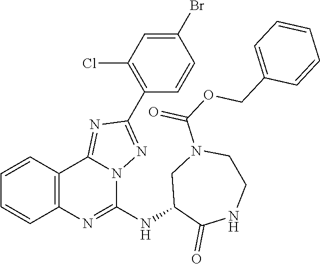 [SMILES: O=C1NCCN(C(=O)OCc2ccccc2)C[C@H]1Nc1nc2ccccc2c2nc(-c3ccc(Br)cc3Cl)nn12] |
| | benzyl (6R)-6-{[2-(4-bromo-2-chlorophenyl) |
| | [1,2,4]triazolo[1,5- |
| | c]quinazolin-5-yl]amino}-5-oxo-1,4- |
| | diazepane-1-carboxylate |
| | LC-MS (Method 2): Rₜ = 1.52 min; MS (ESIpos): |
| | m/z = 620 [M + H]⁺ |
| Intermediate 351 | 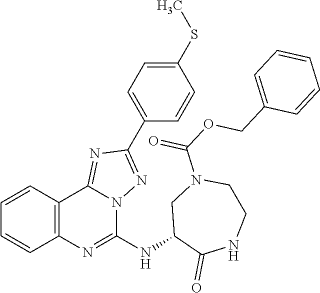 [SMILES: CSc1ccc(-c2nc3c4ccccc4nc(N[C@@H]4CN(C(=O)OCc5ccccc5)CCNC4=O)n3n2)cc1] |
| | benzyl (6R)-6-({2-[4-(methylsulfanyl)phenyl] |
| | [1,2,4]triazolo[1,5- |
| | c]quinazolin-5-yl}amino)-5-oxo-1,4- |
| | diazepane-1-carboxylate |
| | LC-MS (Method 2): Rₜ = 1.47 min; |
| | MS (ESIpos): m/z = 554 [M + H]⁺ |
| Intermediate 352 | 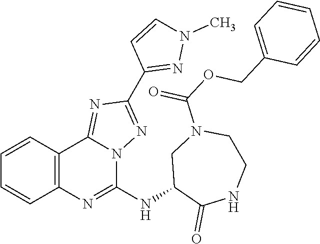 [SMILES: Cn1ccc(-c2nc3c4ccccc4nc(N[C@@H]4CN(C(=O)OCc5ccccc5)CCNC4=O)n3n2)n1] |
| | benzyl (6R)-6-{[2-(1-methyl-1H-pyrazol-3-yl) |
| | [1,2,4]triazolo[1,5- |
| | c]quinazolin-5-yl]amino}-5-oxo- |
| | 1,4-diazepane-1-carboxylate |
| | LC-MS (Method 1): Rₜ = 1.10 min; |
| | MS (ESIpos): m/z = 512 [M + H]⁺ |
| | ¹H-NMR (400 MHz, DMSO-d₆): δ [ppm]= |
| | 2.53 (d, 1H), 2.92-3.12 (m, 1H), |
| | 3.14-3.31 (m, 2H), 3.44-3.54 (m, 1H), 3.99 |
| | (s, 3H), 4.18 (br d, 1H), 4.62 |
| | (br d, 1H), 4.94 (br s, 1H), 5.20 (s, 2H), 6.92 |
| | (d, 1H), 7.13-7.30 (m, 2H), |
| | 7.30-7.50 (m, 5H), 7.60-7.78 (m, 1H), 7.84 (d, 1H), |
| | 7.90 (d, 1H), 8.28 (br |
| | d, 1H), 8.38-8.56 (m, 1H). |
| Intermediate 353 | 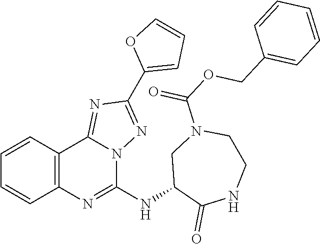 [SMILES: O=C1NCCN(C(=O)OCc2ccccc2)C[C@H]1Nc1nc2ccccc2c2nc(-c3ccco3)nn12] |
| | benzyl (6R)-6-{[2-(furan-2-yl)[1,2,4]triazolo |
| | [1,5-c]quinazolin-5-yl]amino}- |
| | 5-oxo-1,4-diazepane-1-carboxylate |
| | LC-MS (Method 2): Rt = 1.23 min; |
| | MS (ESIpos): m/z = 498 [M + H]⁺ |
| | ¹H-NMR (400 MHz, DMSO-d₆): δ [ppm] = |
| | 3.00 (br d, 1H), 3.17-3.32 (m, 2H), |
| | 3.43-3.54 (m, 1H), 4.21 (br s, 1H), 4.62 (br d, 1H), |
| | 4.93 (br s, 1H), 5.20 (br |
| | s, 2H), 6.76 (dd, 1H), 7.20 (br s, 1H), |
| | 7.30-7.50 (m, 6H), 7.65 (br s, 1H), |
| | 7.85 (d, 1H), 7.99 (dd, 1H), 8.27 (br d, 1H), |
| | 8.40-8.55 (m, 1H). |
| Intermediate 354 | 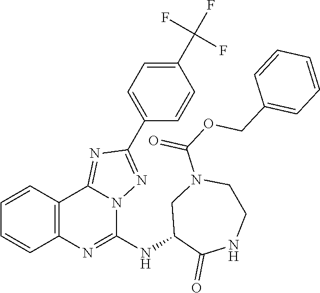 [SMILES: O=C1NCCN(C(=O)OCc2ccccc2)C[C@H]1Nc1nc2ccccc2c2nc(-c3ccc(C(F)(F)F)cc3)nn12] |
| | benzyl (6R)-5-oxo-6-({2-[4-(trifluoromethyl) |
| | phenyl][1,2,4]triazolo[1,5- |
| | c]quinazolin-5-yl}amino)-1,4- |
| | diazepane-1-carboxylate |
| | LC-MS (Method 2): Rt = 1.50 min; |
| | MS (ESIpos): m/z = 576 [M + H]⁺ |
| | ¹H-NMR (400 MHz, DMSO-d₆): δ [ppm] = |
| | 2.99 (s, 1H), 3.49 (br s, 1H), 4.20 |
| | (br s, 1H), 4.64 (br s, 1H), 4.96 (br s, 1H), |
| | 5.20 (br s, 2H), 7.19 (br s, 2H), |
| | 7.28-7.53 (m, 6H), 7.67 (br s, 1H), 7.95 (s, 3H), |
| | 7.99 (br d, 3H), 8.24-8.47 |
| | (m, 3H), 8.50 (br d, 3H). |
| Intermediate 355 | 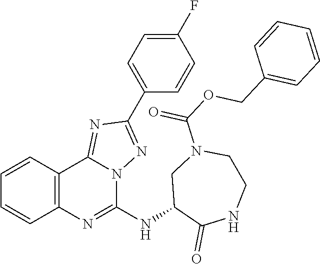 [SMILES: O=C1NCCN(C(=O)OCc2ccccc2)C[C@H]1Nc1nc2ccccc2c2nc(-c3ccc(F)cc3)nn12] |
| | benzyl (6R)-6-{[2-(4-fluorophenyl)[1,2,4] |
| | triazolo[1,5-c]quinazolin-5- |
| | yl]amino}-5-oxo-1,4-diazepane-1-carboxylate |
| | LC-MS (Method 2): Rt = 1.39 min; |
| | MS (ESIpos): m/z = 526 [M + H]⁺ |
| | ¹H-NMR (400 MHz, DMSO-d₆): δ [ppm] = |
| | 2.93-3.07 (m, 1H), 3.29 (br s, 2H), |
| | 3.44-3.56 (m, 1H), 4.21 (br s, 1H), 4.62 (br d, 1H), |
| | 4.95 (br s, 1H), 5.20 (br |
| | s, 2H), 7.19 (br s, 2H), 7.31-7.51 (m, 7H), |
| | 7.65 (br s, 1H), 7.88 (d, 1H), |
| | 8.28-8.37 (m, 3H), 8.38-8.55 (m, 1H). |
| Intermediate 356 | 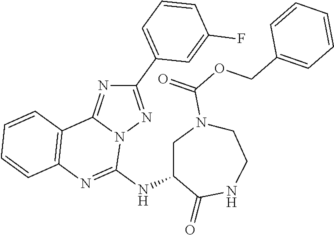 [SMILES: O=C1NCCN(C(=O)OCc2ccccc2)C[C@H]1Nc1nc2ccccc2c2nc(-c3cccc(F)c3)nn12] |
| | benzyl (6R)-6-{[2-(3-fluorophenyl)[1,2,4] |
| | triazolo[1,5-c]quinazolin-5- |
| | yl]amino}-5-oxo-1,4-diazepane-1-carboxylate |
| | LC-MS (Method 2): Rt = 1.40 min; |
| | MS (ESIpos): m/z = 526 [M + H]⁺ |
| | ¹H-NMR (400 MHz, DMSO-d₆): δ [ppm] = |
| | 2.94-3.16 (m, 1H), 3.29 (br d, 2H), |
| | 3.45-3.55 (m, 1H), 4.20 (br s, 1H), 4.62 (br s, 1H), |
| | 4.95 (br s, 1H), 5.20 (br |
| | s, 2H), 7.30-7.53 (m, 6H), 7.66 (td, 1H), |
| | 7.92 (br d, 1H), 8.00 (br d, 1H), |
| | 8.14 (br d, 1H), 8.31 (br d, 1H), 8.37-8.55 (m, 1H). |
| Intermediate 357 | 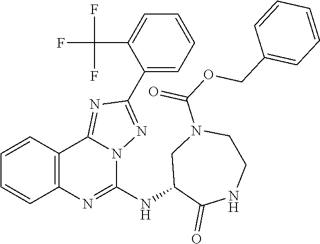 [SMILES: O=C1NCCN(C(=O)OCc2ccccc2)C[C@H]1Nc1nc2ccccc2c2nc(-c3ccccc3C(F)(F)F)nn12] |
| | benzyl (6R)-5-oxo-6-({2-[2-(trifluoromethyl) |
| | phenyl][1,2,4]triazolo[1,5- |
| | c]quinazolin-5-yl}amino)- |
| | 1,4-diazepane-1-carboxylate |
| | LC-MS (Method 2): Rt = 1.39 min; |
| | MS (ESIpos): m/z = 576 [M + H]⁺ |
| | ¹H-NMR (400 MHz, DMSO-d₆): δ [ppm] = |
| | 3.21-3.32 (m, 2H), 3.42-3.55 (m, |
| | 1H), 4.22 (br s, 1H), 4.64 (br d, 1H), 4.93 |
| | (br s, 1H), 5.21 (br s, 2H), 7.28- |
| | 7.51 (m, 5H), 7.67 (br s, 1H), 7.80-7.84 (m, 1H), |
| | 7.85-7.91 (m, 2H), 7.96- |
| | 8.04 (m, 2H), 8.27 (br d, 1H), 8.37-8.55 (m, 1H). |
| Intermediate 358 | 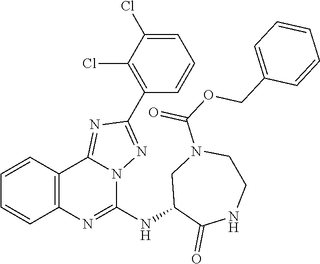 [SMILES: O=C1NCCN(C(=O)OCc2ccccc2)C[C@H]1Nc1nc2ccccc2c2nc(-c3cccc(Cl)c3Cl)nn12] |
| | benzyl (6R)-6-{[2-(2,3-dichlorophenyl) |
| | [1,2,4]triazolo[1,5-c]quinazolin-5- |
| | yl]amino}-5-oxo-1,4-diazepane-1-carboxylate |
| | LC-MS (Method 2): Rₜ = 1.45 min; |
| | MS (ESIpos): m/z = 576 [M + H]⁺ |
| | ¹H-NMR (400 MHz, DMSO-d₆): δ [ppm] = |
| | 2.91-3.12 (m, 1H), 3.18-3.31 (m, |
| | 2H), 3.43-3.55 (m, 1H), 4.10-4.27 (m, 1H), |
| | 4.57-4.72 (m, 1H), 4.88-5.00 |
| | (m, 1H), 5.20 (br s, 2H), 7.14-7.52 (m, 6H), |
| | 7.59 (t, 1H), 7.63-7.83 (m, |
| | 1H), 7.89 (dd, 1H), 7.92 (br d, 1H), 7.95-8.01 |
| | (m, 1H), 8.29 (br d, 1H), 8.37-8.54 (m, 1H). |
| Intermediate 359 | 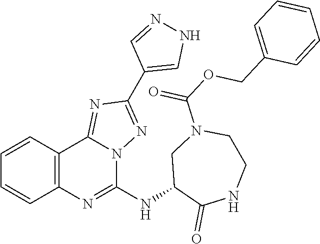 [SMILES: O=C1NCCN(C(=O)OCc2ccccc2)C[C@H]1Nc1nc2ccccc2c2nc(-c3cn[nH]c3)nn12] |
| | benzyl (6R)-5-oxo-6-{[2-(1H-pyrazol-4-yl) |
| | [1,2,4]triazolo[1,5-c]quinazolin- |
| | 5-yl]amino}-1,4-diazepane-1-carboxylate |
| | LC-MS (Method 2): Rt = 1.03 min; |
| | MS (ESIpos): m/z = 498 [M + H]⁺ |
| | ¹H-NMR (400 MHz, DMSO-d₆): δ [ppm] = |
| | 2.52-2.55 (m, 1H), 2.98 (br s, 1H), |
| | 3.16-3.30 (m, 2H), 3.43-3.54 (m, 1H), 4.20 |
| | (br s, 1H), 4.60 (br d, 1H), 4.94 |
| | (br s, 1H), 5.19 (s, 2H), 7.18 (br s, 2H), 7.35 |
| | (br s, 2H), 7.43 (br d, 3H), 7.63 |
| | (br s, 1H), 7.75 (br d, 1H), 8.16 (br s, 1H), |
| | 8.26 (br d, 2H), 8.48 (br s, 2H), |
| | 13.36 (br s, 1H). |
| Intermediate 360 | 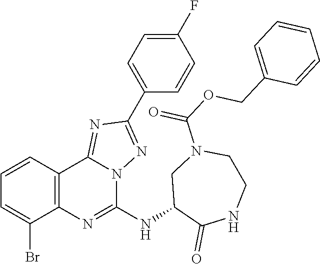 [SMILES: O=C1NCCN(C(=O)OCc2ccccc2)C[C@H]1Nc1nc2c(Br)cccc2c2nc(-c3ccc(F)cc3)nn12] |
| | benzyl (6R)-6-{[7-bromo-2-(4-fluorophenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.46 min; |
| | MS (ESIpos): m/z = 604 [M + H]⁺ |
| | ¹H-NMR (400 MHz, DMSO-d₆): δ [ppm] = |
| | 3.03-3.22 (m, 1H), 3.36-3.48 (m, |
| | 2H), 3.48-3.63 (m, 1H), 4.11 (br d, 1H), |
| | 4.32-4.49 (m, 1H), 4.92-5.20 (m, |
| | 3H), 6.99-7.41 (m, 6H), 7.42-7.49 (m, 2H), |
| | 8.06 (dd, 1H), 8.25-8.41 (m, |
| | 5H). |
| Intermediate 361 | 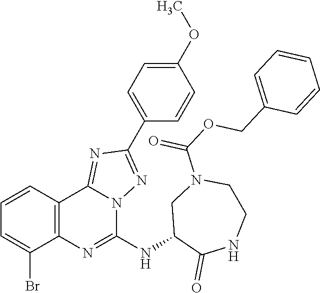 [SMILES: COc1ccc(-c2nc3c4cccc(Br)c4nc(N[C@@H]4CN(C(=O)OCc5ccccc5)CCNC4=O)n3n2)cc1] |
| | benzyl (6R)-6-{[7-bromo-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.42 min; |
| | MS (ESIpos): m/z = 616 [M + H]⁺ |
| | ¹H-NMR (400 MHz, DMSO-d₆): δ [ppm] = |
| | 2.52-2.60 (m, 3H), 3.06-3.30 (m, |
| | 1H), 3.36-3.48 (m, 1H), 3.57 (br s, 1H), |
| | 3.86 (s, 3H), 4.11 (br d, 1H), 4.39 |
| | (br s, 1H), 5.12 (br d, 3H), 6.96-7.26 (m, 5H), |
| | 7.35 (br t, 3H), 8.04 (d, 1H), |
| | 8.24 (d, 4H), 8.28-8.40 (m, 2H). |
| Intermediate 362 | 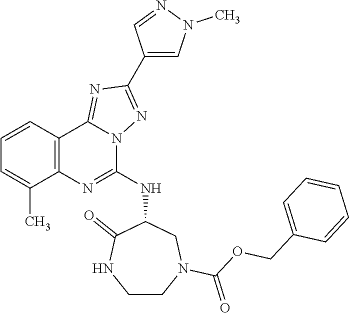 [SMILES: Cc1cccc2c1nc(N[C@@H]1CN(C(=O)OCc3ccccc3)CCNC1=O)n1nc(-c3cnn(C)c3)nc21] |
| | benzyl (6R)-6-{[7-methyl-2-(1-methyl- |
| | 1H-pyrazol-4-yl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.20 min; MS (ESIpos): |
| | m/z = 527 [M + H]⁺ |
| Intermediate 363 | 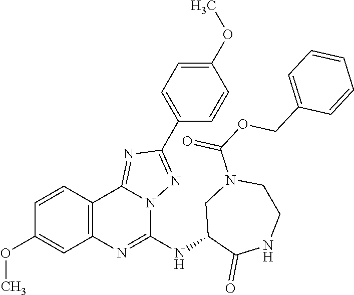 [SMILES: COc1ccc(-c2nc3c4ccc(OC)cc4nc(N[C@@H]4CN(C(=O)OCc5ccccc5)CCNC4=O)n3n2)cc1] |
| | benzyl (6R)-6-{[8-methoxy-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rt = 1.36 min; MS (ESIpos): |
| | m/z = 568 [M + H]⁺ |
| | ¹H-NMR (400 MHz, DMSO-d₆): δ [ppm] = |
| | 1.22-1.31 (m, 3H), 3.86 (s, 3H), |
| | 4.18 (br s, 1H), 4.60 (br d, 1H), 4.98 (br s, 1H), |
| | 5.14-5.28 (m, 2H), 6.95- |
| | 7.08 (m, 2H), 7.12-7.17 (m, 3H), 7.32-7.46 (m, 3H), |
| | 7.85 (d, 1H), 8.20 (t, 3H). |
| Intermediate 364 | 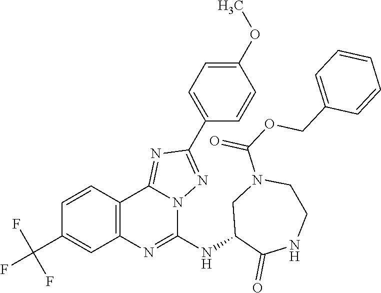 [SMILES: COc1ccc(-c2nc3c4ccc(C(F)(F)F)cc4nc(N[C@@H]4CN(C(=O)OCc5ccccc5)CCNC4=O)n3n2)cc1] |
| | benzyl (6R)-6-{[2-(4-methoxyphenyl)-8- |
| | (trifluoromethyl)[1,2,4]triazolo[1,5-c]quinazolin- |
| | 5-yl]amino}-5-oxo-1,4- |
| | diazepane-1-carboxylate |
| | LC-MS (Method 2): Rt = 1.49 min; |
| | MS (ESIpos): m/z = 606 [M + H]⁺ |
| | ¹H-NMR (400 MHz, DMSO-d₆): δ [ppm] = |
| | 2.95-3.08 (m, 1H), 3.13 (br d, 1H), |
| | 3.44-3.55 (m, 1H), 3.86 (s, 3H), 4.19 (br s , 1H), |
| | 4.61 (br s, 1H), 4.88-5.06 |
| | (m, 1H), 5.11-5.27 (m, 2H), 7.06-7.20 |
| | (m, 4H), 7.28-7.47 (m, 4H), 7.72 |
| | (br s, 1H), 8.09 (d, 1H), 8.24 (d, 2H), 8.42 - 8.53 (m, 2H). |
| Intermediate 365 | 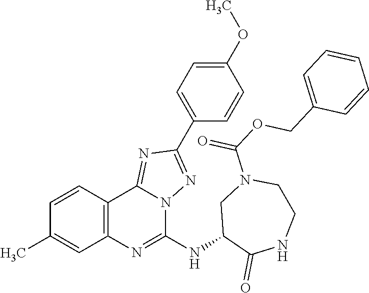 [SMILES: COc1ccc(-c2nc3c4ccc(C)cc4nc(N[C@@H]4CN(C(=O)OCc5ccccc5)CCNC4=O)n3n2)cc1] |
| | benzyl (6R)-6-{[2-(4-methoxyphenyl)- |
| | 8-methyl[1,2,4]triazolo[1,5- |
| | c]quinazolin-5-yl]amino}-5-oxo- |
| | 1,4-diazepane-1-carboxylate |
| | LC-MS (Method 2): Rt = 1.41 min; |
| | MS (ESIpos): m/z = 552 [M + H]⁺ |
| | ¹H-NMR (400 MHz, DMSO-d₆): δ [ppm] = |
| | 2.91-3.17 (m, 1H), 3.43-3.55 (m, |
| | 1H), 3.86 (s, 3H), 4.05-4.29 (m, 1H), |
| | 4.60 (br d, 1H), 4.94 (br s, 1H), 5.21 |
| | (br s, 2H), 7.14 (d, 3H), 7.20-7.50 (m, 6H), |
| | 7.82 (br d, 1H), 8.12-8.26 (m, |
| | 3H), 8.30-8.56 (m, 1H). |
| Intermediate 366 | 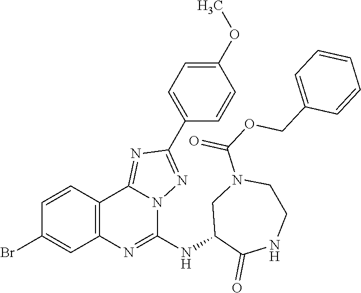 [SMILES: COc1ccc(-c2nc3c4ccc(Br)cc4nc(N[C@@H]4CN(C(=O)OCc5ccccc5)CCNC4=O)n3n2)cc1] |
| | benzyl (6R)-6-{[8-bromo-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rt = 1.49 min; |
| | MS (ESIpos): m/z = 616 [M + H]⁺ |
| | ¹H-NMR (500 MHz, DMSO-d₆): δ [ppm] = |
| | 2.94-3.26 (m, 2H), 3.48 (br t, 1H), |
| | 3.86 (s, 3H), 4.20 (br s, 1H), 4.51-4.68 (m, 1H), |
| | 4.84-5.00 (m, 1H), 5.19 |
| | (s, 2H), 7.12-7.16 (m, 2H), 7.19 (br s, 1H), |
| | 7.31-7.45 (m, 3H), 7.55-7.62 |
| | (m, 1H), 8.00 (d, 1H), 8.21 (br d, 3H), 8.40-8.53 (m, 1H). |
| Intermediate 367 | 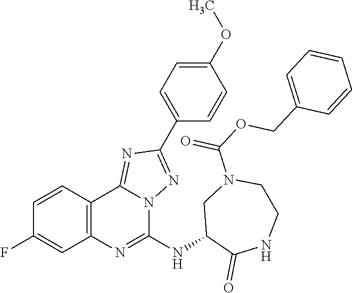 [SMILES: COc1ccc(-c2nc3c4ccc(F)cc4nc(N[C@@H]4CN(C(=O)OCc5ccccc5)CCNC4=O)n3n2)cc1] |
| | benzyl (6R)-6-{[8-fluoro-2-(4-methoxyphenyl) |
| | [1,2,4]triazolo[1,5- |
| | c]quinazolin-5-yl]amino}-5-oxo-1,4- |
| | diazepane-1-carboxylate |
| | LC-MS (Method 2): Rt = |
| | 1.39 min; MS (ESIpos): m/z = 556 [M + H]⁺ |
| | ¹H-NMR (400 MHz, DMSO-d₆): δ [ppm] = |
| | 2.90-3.15 (m, 1H), 3.18-3.30 (m, |
| | 1H), 3.42-3.59 (m, 1H), 3.86 (s, 3H), |
| | 4.20 (br s, 1H), 4.58 (br d, 1H), 4.95 |
| | (br s, 1H), 5.11-5.29 (m, 2H), 7.03-7.22 (m, 4H), |
| | 7.23-7.48 (m, 5H), 7.97 |
| | (d, 1H), 8.22 (br d, 2H), 8.35 (br d, 1H), 8.39-8.58 (m, 1H). |
| Intermediate 368 | 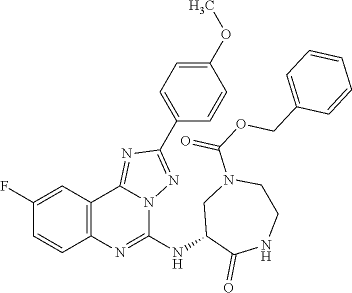 [SMILES: COc1ccc(-c2nc3c4cc(F)ccc4nc(N[C@@H]4CN(C(=O)OCc5ccccc5)CCNC4=O)n3n2)cc1] |
| | benzyl (6R)-6-{[9-fluoro-2- |
| | (4-methoxyphenyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.41 min; |
| | MS (ESIpos): m/z = 556 [M + H]⁺ |
| Intermediate 369 | 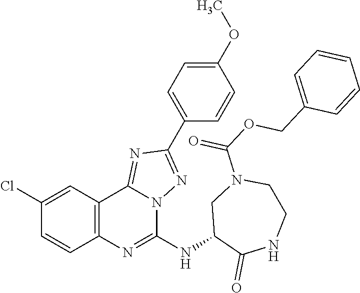 [SMILES: COc1ccc(-c2nc3c4cc(Cl)ccc4nc(N[C@@H]4CN(C(=O)OCc5ccccc5)CCNC4=O)n3n2)cc1] |
| | benzyl (6R)-6-{[9-chloro-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.49 min; |
| | MS (ESIpos): m/z = 572 [M + H]⁺ |
| Intermediate 370 | 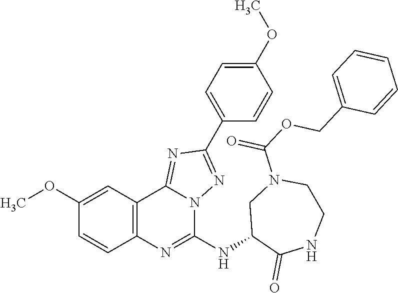 [SMILES: COc1ccc(-c2nc3c4cc(OC)ccc4nc(N[C@@H]4CN(C(=O)OCc5ccccc5)CCNC4=O)n3n2)cc1] |
| | benzyl (6R)-6-{[9-methoxy-2-(4-methoxyphenyl) |
| | [1,2,4]triazolo[1,5- |
| | c]quinazolin-5-yl]amino}- |
| | 5-oxo-1,4-diazepane-1-carboxylate |
| | LC-MS (Method 2): |
| | Rₜ = 1.35 min; MS (ESIpos): m/z = 568 [M + H]⁺ |
| Intermediate 371 | 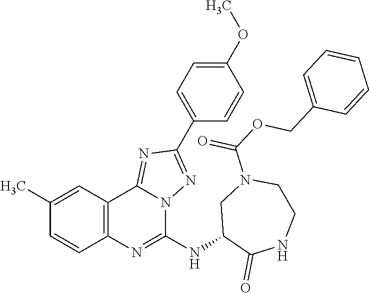 [SMILES: COc1ccc(-c2nc3c4cc(C)ccc4nc(N[C@@H]4CN(C(=O)OCc5ccccc5)CCNC4=O)n3n2)cc1] |
| | benzyl (6R)-6-{[2-(4-methoxyphenyl)-9- |
| | methyl[1,2,4]triazolo[1,5- |
| | c]quinazolin-5-yl]amino}-5-oxo-1,4- |
| | diazepane-1-carboxylate |
| | LC-MS (Method 2): Rₜ = 1.42 min; |
| | MS (ESIpos): m/z = 552 [M + H]⁺ |
| Intermediate 372 | 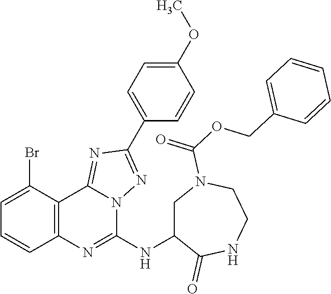 [SMILES: COc1ccc(-c2nc3c4c(Br)cccc4nc(NC4CN(C(=O)OCc5ccccc5)CCNC4=O)n3n2)cc1] |
| | Benzyl-6-{[10-bromo-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.49 min; |
| | MS (ESIpos): m/z = 616 [M + H]⁺ |
| | ¹H-NMR (400 MHz, DMSO-d₆): δ [ppm] = |
| | 2.93-3.12 (m, 1H), 3.19-3.31 (m, |
| | 1H), 3.43-3.55 (m, 1H), 3.86 (s, 3H), |
| | 4.12-4.28 (m, 1H), 4.55-4.74 (m, |
| | 1H), 4.86-4.99 (m, 1H), 5.20 (br s, 2H), |
| | 7.12-7.77 (m, 10H), 7.97 (d, 1H), |
| | 8.24 (br d, 2H), 8.39-8.56 (m, 1H). |
| Intermediate 373 | 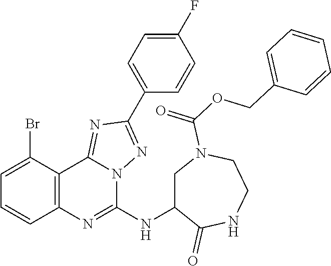 [SMILES: O=C1NCCN(C(=O)OCc2ccccc2)CC1Nc1nc2cccc(Br)c2c2nc(-c3ccc(F)cc3)nn12] |
| | Benzyl-6-{[10-bromo-2-(4-fluorophenyl)[1,2,4] |
| | triazolo[1,5-c]quinazolin- |
| | 5-yl]amino}-5-oxo-1,4-diazepane-1-carboxylate |
| | LC-MS (Method 2): Rₜ = 1.53 min; |
| | MS (ESIpos): m/z = 604 [M + H]⁺ |
| | ¹H-NMR (400 MHz, DMSO-d₆): δ [ppm] = |
| | 2.90-3.12 (m, 1H), 3.17-3.31 (m, |
| | 1H), 3.43-3.56 (m, 1H), 4.11-4.28 (m, 1H), |
| | 4.55-4.73 (m, 1H), 4.93 (br d, |
| | 1H), 5.20 (br s, 2H), 7.14-7.76 (m, 11H), |
| | 8.00 (d, 1H), 8.29-8.38 (m, 2H), |
| | 8.41-8.58 (m, 1H). |
| Intermediate 374 | 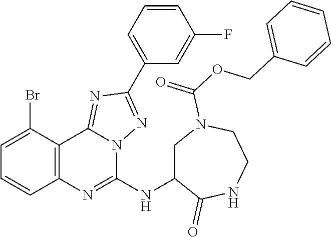 [SMILES: O=C1NCCN(C(=O)OCc2ccccc2)CC1Nc1nc2cccc(Br)c2c2nc(-c3cccc(F)c3)nn12] |
| | benzyl-6-{[10-bromo-2-(3-fluorophenyl) |
| | [1,2,4]triazolo[1,5-c]quinazolin- |
| | 5-yl]amino}-5-oxo-1,4-diazepane-1-carboxylate |
| | LC-MS (Method 2): Rₜ = 1.53 min; |
| | MS (ESIpos): m/z = 604 [M + H]⁺ |
| | ¹H-NMR (400 MHz, DMSO-d₆): δ [ppm] = |
| | 2.92-3.14 (m, 1H), 3.19-3.31 (m, |
| | 1H), 3.44-3.56 (m, 1H), 4.12-4.28 (m, 1H), |
| | 4.55-4.73 (m, 1H), 4.86-5.01 |
| | (m, 1H), 5.20 (br s, 2H), 7.13-7.78 (m, 10H), |
| | 7.96-8.06 (m, 2H), 8.15 (br |
| | d, 1H), 8.39-8.57 (m, 1H). |
| Intermediate 375 | 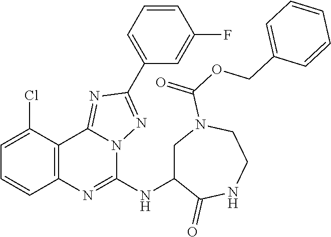 [SMILES: O=C1NCCN(C(=O)OCc2ccccc2)CC1Nc1nc2cccc(Cl)c2c2nc(-c3cccc(F)c3)nn12] |
| | benzyl-6-{[10-chloro-2-(3-fluorophenyl) |
| | [1,2,4]triazolo[1,5-c]quinazolin- |
| | 5-yl]amino}-5-oxo-1,4-diazepane-1-carboxylate |
| | LC-MS (Method 2): Rₜ = 1.50 min; |
| | MS (ESIpos): m/z = 560 [M + H]⁺ |
| | ¹H-NMR (400 MHz, DMSO-d₆): δ [ppm] = |
| | 2.93-3.13 (m, 1H), 3.18-3.31 (m, |
| | 1H), 3.44-3.56 (m, 1H), 4.12-4.29 (m, 1H), |
| | 4.54-4.72 (m, 1H), 4.88-5.00 |
| | (m, 1H), 5.20 (br s, 2H), 7.14-7.76 (m, 10H), |
| | 8.00 (br d, 1H), 8.05 (d, 1H), |
| | 8.15 (br d, 1H), 8.40-8.56 (m, 1H). |
| Intermediate 376 | 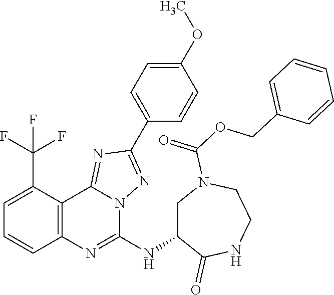 [SMILES: COc1ccc(-c2nc3c4c(C(F)(F)F)cccc4nc(N[C@@H]4CN(C(=O)OCc5ccccc5)CCNC4=O)n3n2)cc1] |
| | benzyl (6R)-6-{[2-(4-methoxyphenyl)-10- |
| | (trifluoromethyl)[1,2,4]triazolo[1,5-c]quinazolin- |
| | 5-yl]amino}-5-oxo-1,4- |
| | diazepane-1-carboxylate |
| | LC-MS (Method 2): Rₜ = 1.50 min; |
| | MS (ESIpos): m/z = 606 [M + H]⁺ |
| | ¹H-NMR (400 MHz, DMSO-d₆): δ [ppm] = |
| | 2.94-3.15 (m, 1H), 3.44-3.56 (m, |
| | 1H), 3.86 (s, 3H), 4.12-4.27 (m, 1H), |
| | 4.54-4.74 (m, 1H), 4.88-5.00 (m, |
| | 1H), 5.20 (s, 2H), 7.12-7.47 (m, 7H), |
| | 7.61-7.96 (m, 3H), 8.04 (d, 1H), 8.22 |
| | (br d, 2H), 8.39-8.55 (m, 1H). |
| Intermediate 377 | 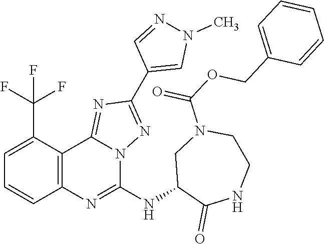 [SMILES: Cn1cc(-c2nc3c4c(C(F)(F)F)cccc4nc(N[C@@H]4CN(C(=O)OCc5ccccc5)CCNC4=O)n3n2)cn1] |
| | benzyl (6R)-6-{[2-(1-methyl-1H-pyrazol-4-yl)-10- |
| | (trifluoromethyl)[1,2,4]triazolo[1,5-c] |
| | quinazolin-5-yl]amino}-5-oxo-1,4- |
| | diazepane-1-carboxylate |
| | LC-MS (Method 2): Rₜ = 1.28 min; MS (ESIpos): |
| | m/z = 580 [M + H]⁺ |
| Intermediate 378 | 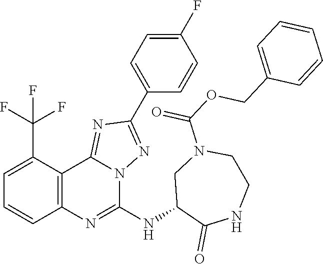 [SMILES: O=C1NCCN(C(=O)OCc2ccccc2)C[C@H]1Nc1nc2cccc(C(F)(F)F)c2c2nc(-c3ccc(F)cc3)nn12] |
| | benzyl (6R)-6-{[2-(4-fluorophenyl)- |
| | 10-(trifluoromethyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.53 min; |
| | MS (ESIpos): m/z = 594 [M + H]⁺ |
| Intermediate 379 | 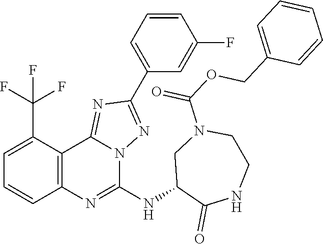 [SMILES: O=C1NCCN(C(=O)OCc2ccccc2)C[C@H]1Nc1nc2cccc(C(F)(F)F)c2c2nc(-c3cccc(F)c3)nn12] |
| | benzyl (6R)-6-{[2-(3-fluorophenyl)-10- |
| | (trifluoromethyl)[1,2,4]triazolo[1,5- |
| | c]quinazolin-5-yl]amino}-5-oxo-1,4- |
| | diazepane-1-carboxylate |
| | LC-MS (Method 2): Rₜ = 1.53 min; |
| | MS (ESIpos): m/z = 594 [M + H]⁺ |
| | ¹H-NMR (400 MHz, DMSO-d₆): δ [ppm] = |
| | 2.93-3.18 (m, 1H), 3.44-3.57 (m, |
| | 1H), 4.14-4.30 (m, 1H), 4.55-4.73 (m, 1H), |
| | 4.87-5.02 (m, 1H), 5.20 (s, |
| | 2H), 7.12-7.50 (m, 7H), 7.63-8.01 (m, 5H), |
| | 8.07-8.16 (m, 2H), 8.40-8.57 |
| | (m, 1H). |
| Intermediate 380 | 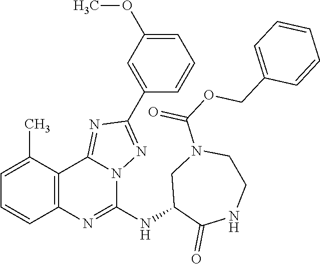 [SMILES: COc1cccc(-c2nc3c4c(C)cccc4nc(N[C@@H]4CN(C(=O)OCc5ccccc5)CCNC4=O)n3n2)c1] |
| | benzyl (6R)-6-{[2-(3-methoxyphenyl)- |
| | 10-methyl[1,2,4]triazolo[1,5- |
| | c]quinazolin-5-yl]amino}-5-oxo- |
| | 1,4-diazepane-1-carboxylate |
| | LC-MS (Method 2): Rₜ = 1.53 min; |
| | MS (ESIpos): m/z = 552 [M + H]⁺ |
| | ¹H-NMR (400 MHz, DMSO-d₆): δ [ppm] = |
| | 2.94-3.16 (m, 1H), 3.03 (s, 3H), |
| | 3.44-3.55 (m, 1H), 3.88 (s, 3H), |
| | 4.11-4.28 (m, 1H), 4.54-4.69 (m, 1H), |
| | 4.88-5.00 (m, 1H), 5.19 (br s, 2H), |
| | 7.12-7.23 (m, 3H), 7.25-7.45 (m, 5H), |
| | 7.46-7.56 (m, 2H), 7.81 (br s, 1H), 7.85-7.93 |
| | (m, 2H), 8.36-8.52 (m, 1H). |
| Intermediate 381 | 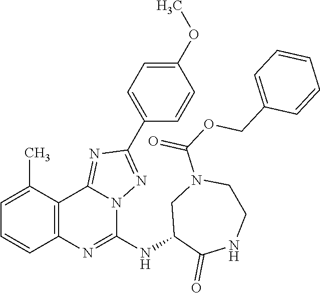 [SMILES: COc1ccc(-c2nc3c4c(C)cccc4nc(N[C@@H]4CN(C(=O)OCc5ccccc5)CCNC4=O)n3n2)cc1] |
| | benzyl (6R)-6-{[2-(4-methoxyphenyl)- |
| | 10-methyl[1,2,4]triazolo[1,5- |
| | c]quinazolin-5-yl]amino}-5-oxo- |
| | 1,4-diazepane-1-carboxylate |
| | LC-MS (Method 2): Rₜ = 1.52 min; |
| | MS (ESIpos): m/z = 552 [M + H]⁺ |
| Intermediate 382 | 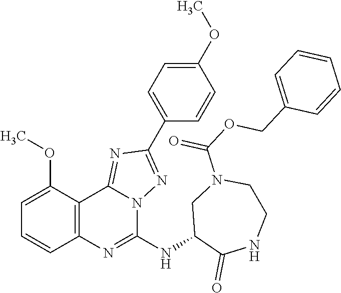 [SMILES: COc1ccc(-c2nc3c4c(OC)cccc4nc(N[C@@H]4CN(C(=O)OCc5ccccc5)CCNC4=O)n3n2)cc1] |
| | benzyl (6R)-6-{[10-methoxy-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.25 min; |
| | MS (ESIpos): m/z = 568 [M + H]⁺ |
| Intermediate 383 | 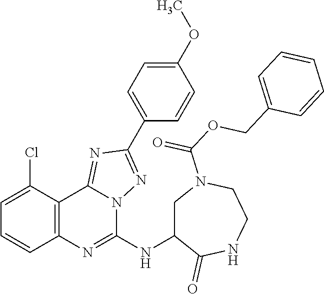 [SMILES: COc1ccc(-c2nc3c4c(Cl)cccc4nc(NC4CN(C(=O)OCc5ccccc5)CCNC4=O)n3n2)cc1] |
| | Benzyl-6-{[10-chloro-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.46 min; |
| | MS (ESIpos): m/z = 572 [M + H]⁺ |
| | ¹H-NMR (400 MHz, DMSO-d₆): δ [ppm] = |
| | 2.92-3.14 (m, 1H), 3.23-3.32 (m, |
| | 1H), 3.43-3.55 (m, 1H), 3.86 (s, 3H), |
| | 4.12-4.27 (m, 1H), 4.54-4.74 (m, |
| | 1H), 4.87-4.99 (m, 1H), 5.20 (br s, 2H), |
| | 7.13-7.18 (m, 2H), 7.19-7.74 (m, |
| | 8H), 7.98 (d, 1H), 8.18-8.27 (m, 2H), |
| | 8.37- 8.56 (m, 1H). |
| Intermediate 384 | 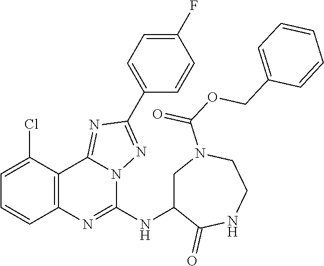 [SMILES: O=C1NCCN(C(=O)OCc2ccccc2)CC1Nc1nc2cccc(Cl)c2c2nc(-c3ccc(F)cc3)nn12] |
| | Benzyl-6-{[10-chloro-2-(4-fluorophenyl) |
| | [1,2,4]triazolo[1,5-c]quinazolin- |
| | 5-yl]amino}-5-oxo-1,4-diazepane-1-carboxylate |
| | LC-MS (Method 2): Rₜ = 1.49 min; |
| | MS (ESIpos): m/z = 560 [M + H]⁺ |
| | ¹H-NMR (400 MHz, DMSO-d₆): δ [ppm] = |
| | 2.93-3.12 (m, 1H), 3.18-3.31 (m, |
| | 1H), 3.44-3.56 (m, 1H), 4.13-4.28 (m, 1H), |
| | 4.55-4.74 (m, 1H), 4.88-5.00 |
| | (m, 1H), 5.20 (br s, 2H), 7.14-7.76 (m, 11H), |
| | 8.01 (d, 1H), 8.29-8.38 (m, |
| | 2H), 8.42-8.56 (m, 1H). |
| Intermediate 385 | 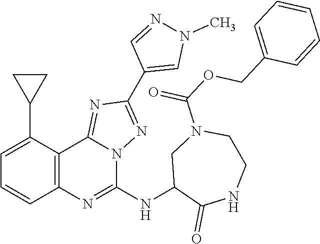 [SMILES: Cn1cc(-c2nc3c4c(C5CC5)cccc4nc(NC4CN(C(=O)OCc5ccccc5)CCNC4=O)n3n2)cn1] |
| | Benzyl-6-{[10-cyclopropyl-2- |
| | (1-methyl-1H-pyrazol-4- |
| | yl)[1,2,4]triazolo[1,5-c]quinazolin-5-yl]amino}- |
| | 5-oxo-1,4-diazepane-1- |
| | carboxylate |
| | LC-MS (Method 2): Rₜ = 1.34 min; |
| | MS (ESIpos): m/z = 553 [M + H]⁺ |
| | ¹H-NMR (400 MHz, DMSO-d₆): δ [ppm] = |
| | 0.81-0.89 (m, 2H), 1.15-1.22 (m, |
| | 2H), 2.91-3.11 (m, 1H), 3.21-3.31 (m, 2H), |
| | 3.43-3.55 (m, 1H), 3.80 (tt, |
| | 1H), 3.95 (s, 3H), 4.11-4.27 (m, 1H), |
| | 4.54-4.71 (m, 1H), 4.85-4.98 (m, |
| | 1H), 5.19 (br s, 2H), 6.95 (br d, 1H), |
| | 7.13-7.64 (m, 7H), 7.72 (br d, 1H), |
| | 8.06 (s, 1H), 8.37-8.54 (m, 2H). |
| Intermediate 386 | 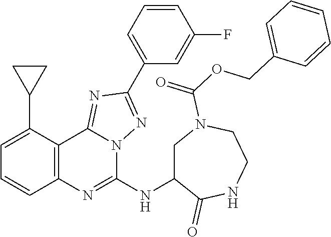 [SMILES: O=C1NCCN(C(=O)OCc2ccccc2)CC1Nc1nc2cccc(C3CC3)c2c2nc(-c3cccc(F)c3)nn12] |
| | Benzyl-6-{[10-cyclopropyl-2-(3-fluorophenyl) |
| | [1,2,4]triazolo[1,5- |
| | c]quinazolin-5-yl]amino}-5-oxo-1,4- |
| | diazepane-1-carboxylate |
| | LC-MS (Method 2): Rₜ = 1.60 min; |
| | MS (ESIpos): m/z = 566 [M + H]⁺ |
| | ¹H-NMR (400 MHz, DMSO-d₆): δ [ppm] = |
| | 0.83-0.90 (m, 2H), 1.19-1.27 (m, |
| | 2H), 2.94-3.14 (m, 1H), 3.18-3.32 (m, 2H), |
| | 3.44-3.56 (m, 1H), 3.74-3.84 |
| | (m, 1H), 4.13-4.27 (m, 1H), 4.54-4.72 (m, 1H), |
| | 4.87-5.01 (m, 1H), 5.19 |
| | (br s, 2H), 6.99 (br d, 1H), 7.13-7.70 (m, 9H), |
| | 7.90 (d, 1H), 8.01 (br d, 1H), |
| | 8.14 (br d, 1H), 8.38-8.54 (m, 1H). |

### Intermediate 387

### Benzyl (6R)-5-oxo-6-({2-[2-(trifluoromethoxy)phenyl][1,2,4]triazolo[1,5-c]quinazolin-5-yl}amino)-1,4-diazepane-1-carboxylate

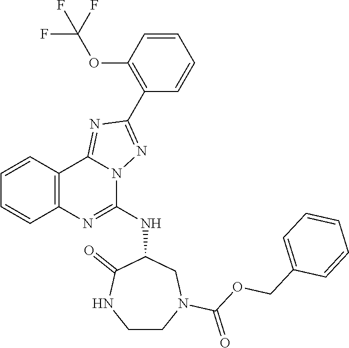 [SMILES: O=C1NCCN(C(=O)OCc2ccccc2)C[C@H]1Nc1nc2ccccc2c2nc(-c3ccccc3OC(F)(F)F)nn12]

5-Chloro-2-[2-(trifluoromethoxy)phenyl][1,2,4]triazolo[1,5-c]quinazoline (87.0 mg, 239 μmol) was suspended in DMSO (0.95 mL). Benzyl (6R)-6-amino-5-oxo-1,4-diazepane-1-carboxylate hydrochloride (1:1) (107 mg, 358 μmol) and N,N-diisopropylethylamine (125 μL, 720 μmol) were added. It was stirred at 60° C. for 2 h. The reaction mixture was allowed to cool down and the solid was filtered off, washed with DMSO (2×0.5 mL) and twice with water. It was dried under vacuum at 50° C. affording 67.5 mg (48%) of the title product which was used without further purification in the next step. The filtrate was purified by HPLC to yield 23 mg (16%) of the title product.

LC-MS (Method 2): Rₜ=1.47 min; MS (ESIpos): m/z=592 [m+H]⁺

[α]²⁰_{D}: −83.2° (c=1.00, DMSO)

¹H-NMR (400 MHz, DMSO-d₆): δ [ppm]=2.91-3.11 (m, 1H), 3.11-3.32 (m, 2H and water signal), 3.45-3.56 (m, 1H), 4.12-4.30 (m, 1H), 4.63-4.81 (m, 1H), 4.87-4.99 (m, 1H), 5.22 (br s, 2H), 7.13-7.53 (m, 6H), 7.59-7.69 (m, 3H), 7.69-7.90 (m, 3H), 8.29 (br d, 1H), 8.37 (dd, 1H), 8.41-8.56 (m, 1H).

### Intermediate 388

### (3R)-3-{[2-(4-methoxyphenyl)-7-(prop-1-en-2-yl)[1,2,4]triazolo[1,5-c]quinazolin-5-yl]amino}azepan-2-one

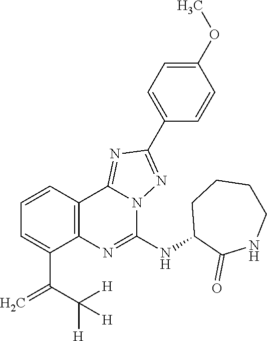 [SMILES: C=C(C)c1cccc2c1nc(N[C@@H]1CCCCNC1=O)n1nc(-c3ccc(OC)cc3)nc21]

(3R)-3-{[7-bromo-2-(4-methoxyphenyl)[1,2,4]triazolo[1,5-c]quinazolin-5-yl]amino}azepan-2-one (100 mg, 208 μmol) and XPhosPd G4 (89.4 mg, 104 μmol) were dissolved in 1,4-dioxane (5.0 ml), aq. Na₂CO₃ (270 μl, 2.0 M, 540 μmol) was added and the mixture was degassed by bubbling argon for 5 minutes. After degassing, 4,4,5,5-tetramethyl-2-(prop-1-en-2-yl)-1,3,2-dioxaborolane (69.8 mg, 415 μmol) was added and the reaction tube was sealed. The mixture stirred 2 h at 100° C. The reaction mixture was cooled to rt and water was added and the mixture was extracted with EtOAc. The organic layer was dried (silicon filter) and concentrated under reduced pressure. The crude material was purified by preparative HPLC to give 34.2 mg (95% purity, 35% yield) of the title compound LC-MS (method 2): Rₜ=1.49 min; MS (ESIpos): m/z=443 [M+H]⁺

¹H-NMR (400 MHz, DMSO-d6): δ [ppm]=1.21-1.40 (m, 2H), 1.49-1.64 (m, 1H), 1.80-1.96 (m, 2H), 1.97-2.14 (m, 2H), 3.19 (br s, 1H), 3.86 (s, 3H), 4.70-4.88 (m, 1H), 5.20 (s, 1H), 5.30 (s, 1H), 7.14 (d, 2H), 7.36-7.44 (m, 1H), 7.57-7.76 (m, 1H), 7.58-7.94 (m, 1H), 8.14-8.27 (m, 4H).

### Intermediate 389

### (3R)-3-{[2-(4-methoxyphenyl)-7-(3,3,3-trifluoroprop-1-en-2-yl)[1,2,4]triazolo[1,5-c]quinazolin-5-yl]amino}azepan-2-one

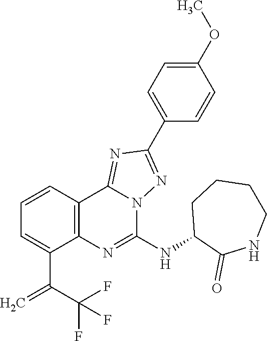 [SMILES: C=C(c1cccc2c1nc(N[C@@H]1CCCCNC1=O)n1nc(-c3ccc(OC)cc3)nc21)C(F)(F)F]

(3R)-3-{[7-bromo-2-(4-methoxyphenyl)[1,2,4]triazolo[1,5-c]quinazolin-5-yl]amino}azepan-2-one (20.0 mg, 41.5 μmol) and Xphos Pd G4 (1.79 mg, 2.08 μmol) were solubilised in 1,4-dioxane (1.0 ml) and aq. Na₂CO₃ (54 μl, 2.0 M, 110 μmol) was added. The mixture was degassed by bubbling with argon for 5 minutes. After degassing 4,4,5,5-tetramethyl-2-(3,3,3-trifluoroprop-1-en-2-yl)-1,3,2-dioxaborolane (18 μl, 83 μmol) was added and the reaction tube was sealed. The mixture was stirred 18 h at 100° C. The reaction was cooled to rt and water was added. The mixture was extracted with EtOAc and the combined organic layer was dried (silicon filter) and concentrated under reduced pressure to give 29 mg of the title compound that was used without further purification.

LC-MS (Method 2): Rₜ=1.48 min; MS (ESIpos): m/z=497 [M+H]⁺

### Intermediate 390

### methyl 3-amino-N-(tert-butoxycarbonyl)-D-alaninate

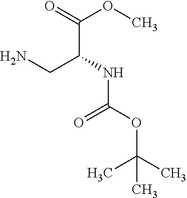

To a solution of 3-amino-N-(tert-butoxycarbonyl)-D-alanine (220 g, 1.08 mol, 1.00 eq) in MeOH (3.60 L) and DCM (360 mL), cooled to 10° C., under nitrogen atmosphere, was added dropwise diazomethyl(trimethyl)silane (2.0 M in n-hexane, 583 mL, 1.08 eq) at 10-20° C. The mixture was stirred at 10-20° C. for 20 h and concentrated under reduced pressure at 25° C. To the residue was added MTBE and stirred for 30 min, filtered and the filtrate was concentrated under reduced pressure at 25° C. to give 290 g (70% purity, 43% yield) of the target compound, which was used without further purification.

1H NMR (CDCl3): δ [ppm]=5.45-5.64 (m, 1H), 4.12-4.22 (m, 1H), 3.64 (s, 3H), 2.93 (d, J=4.4 Hz, 2H), 1.33 (s, 9H).

### Intermediate 391

### 9H-fluoren-9-ylmethyl (2-oxoethyl)carbamate

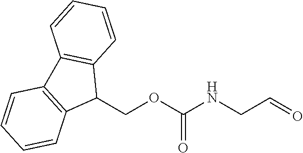 [SMILES: O=CCNC(=O)OCC1c2ccccc2-c2ccccc21]

To a mixture of 9H-fluoren-9-ylmethyl (2-hydroxyethyl)carbamate (200 g, 706 mmol, 1.00 eq) and 2-Iodoxybenzoic acid (260 g, 929 mmol, 1.32 eq) in ethyl acetate (2.00 L) was stirred at 75-78° C. under nitrogen atmosphere. DMSO (110 g, 1.41 mol, 110 mL, 2.00 eq) was added and the mixture was stirred at 75-78° C. for 15 h. The reaction mixture was cooled to 10-20° C. and ethyl acetate (4.00 L) was added. It was stirred for 30 min, filtered and the filtrate was washed with sodium thiosulfate solution (10%), sat. sodium hydrogen carbonate solution and brine. The organic layer was dried and concentrated under reduced pressure. The residue was triturated with MTBE, filtered and the solid was dried under reduced pressure to give 683 g (86% yield) of the target compound, which was used without further purification.

1H NMR (400 MHz, CDCl3): δ [ppm]=9.66 (s, 1H), 7.78 (d, J=7.6 Hz, 2H), 7.61 (d, J=7.6 Hz, 2H), 7.42 (t, J=7.6 Hz, 2H), 7.33 (t, J=7.6 Hz, 2H), 5.48 (s, 1H), 4.44 (d, J=7.2 Hz, 2H), 4.24 (t, J=6.8 Hz, 1H), 4.15 (d, J=5.2 Hz, 2H).

### Intermediate 392

### methyl N-(tert-butoxycarbonyl)-3-[(2-{[(9H-fluoren-9-ylmethoxy)carbonyl]amino}ethyl)amino]-D-alaninate

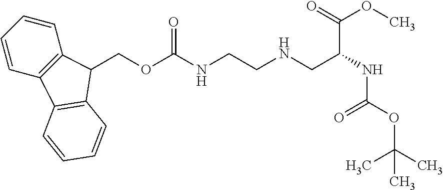 [SMILES: COC(=O)[C@@H](CNCCNC(=O)OCC1c2ccccc2-c2ccccc21)NC(=O)OC(C)(C)C]

To a mixture of 9H-fluoren-9-ylmethyl (2-oxoethyl)carbamate (310 g, 1.10 mol, 1.00 eq) and methyl 3-amino-N-(tert-butoxycarbonyl)-D-alaninate (434 g, 1.37 mol, 1.24 eq) in DCM (3.10 L), cooled to 5° C., was added trimethoxymethane (352 g, 3.31 mol, 363 mL, 3.01 eq) at 5-15° C. and stirred for 1 h at 5-15° C. Sodium triacetoxyborohydride (350 g, 1.65 mol, 1.50 eq) was added to the mixture under nitrogen atmosphere and it was stirred for 1 h at 5-15° C. Sat. sodium hydrogen carbonate solution was added to the mixture and stirred for 1 h. The aqueous phase was extracted with ethyl acetate and the organic phase was dried and concentrated under reduced pressure. The residue was purified by flash chromatography to give 297 g (55% yield) of the target compound.

1H NMR (400 MHz, CDCl3): δ [ppm]=7.76 (d, J=7.2 Hz, 2H), 7.60 (d, J=7.2 Hz, 2H), 7.40 (t, J=7.2 Hz, 2H), 7.31 (t, J=7.2 Hz, 2H), 5.43 (d, J=6.8 Hz, 1H), 5.29 (s, 1H), 4.32-4.52 (m, 3H), 4.22 (t, J=6.4 Hz, 1H), 3.73 (s, 3H), 3.20-3.32 (m, 1H), 3.03-3.12 (m, 1H), 2.91-3.01 (m, 2H), 2.66-2.85 (m, 2H), 1.45 (s, 9H).

### Intermediate 394

### benzyl (6R)-6-[(tert-butoxycarbonyl)amino]-5-oxo-1,4-diazepane-1-carboxylate

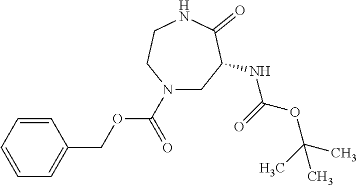 [SMILES: CC(C)(C)OC(=O)N[C@@H]1CN(C(=O)OCc2ccccc2)CCNC1=O]

A mixture of methyl 3-{[(benzyloxy)carbonyl](2-{[(9H-fluoren-9-ylmethoxy)carbonyl]amino}ethyl)amino}-N-(tert-butoxycarbonyl)-D-alaninate (300 g, 486 mmol, 1.00 eq) and piperidine (331 g, 3.89 mol, 384 mL, 8.00 eq) in DMF (3.00 L) was stirred at 15-20° C. for 39 h. The reaction mixture was poured into water and extracted 3 times with ethyl acetate, the combined organic phases were washed 2 times with brine, dried and concentrated under reduced pressure. The residue was purified by flash chromatography to give 127 g (95% purity, 68% yield) of the target compound. 1H NMR (400 MHz, CDCl3): δ [ppm]=7.29-7.62 (m, 5H), 6.59 (s, 1H), 5.82 (s, 1H), 5.22 (s, 2H), 4.18-4.62 (m, 3H), 3.22-3.52 (m, 2H), 2.84-3.05 (m, 2H), 1.49 (s, 9H).

### Intermediate 395

### benzyl (6R)-6-amino-5-oxo-1,4-diazepane-1-carboxylate hydrochloride (1:1)

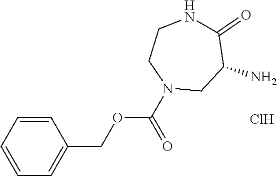 [SMILES: N[C@@H]1CN(C(=O)OCc2ccccc2)CCNC1=O ; Cl]

To a mixture of benzyl (6R)-6-[(tert-butoxycarbonyl)amino]-5-oxo-1,4-diazepane-1-carboxylate (127 g, 349 mmol, 1.00 eq) in dioxane (500 mL) was added hydrogen chloride (4.0 M in dioxane, 437 mL, 5.00 eq) at 15-25° C. and the mixture was stirred at 15-25° C. for 21 h. MTBE was added to the reaction mixture, it was stirred for 15 min, filtered and the solid was dried under reduced pressure to give 103.06 g (100% purity, 98% yield) of the target compound.

1H NMR (400 MHz, DMSO-d6): δ [ppm]=8.45 (s, 4H), 7.25-7.51 (m, 5H), 5.14 (s, 2H), 4.25 (d, J=12.0 Hz, 2H), 4.10 (d, J=12.8 Hz, 1H), 3.36-3.45 (m, 1H), 3.10-3.29 (m, 2H), 2.82-3.07 (br s, 1H).

### Intermediate 396

### ethyl 2-(difluoromethoxy)benzoate

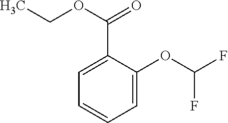

Thionyl dichloride (160 μL, 2.2 mmol) was added dropwise to 2-(difluoromethoxy)benzoic acid (100 mg, 532 μmol) under argon, DCM (0.5 mL) was added and it was stirred for 30 min at rt. The mixture was cooled to 0° C. and ethanol (880 μL) was added dropwise. The mixture was stirred for 30 min at 0° C., 1 h at reflux and overnight at rt. The mixture was concentrated under reduced pressure to give 95.0 mg (83% yield) of the target compound, which was used without further purification.

LC-MS (Method 1): Rₜ=1.17 min; MS (ESIpos): m/z=217 [M+H]⁺

¹H-NMR (400 MHz, DMSO-d₆): δ [ppm]=1.30 (t, 3H), 4.30 (q, 2H), 7.18 (t, 1H), 7.32 (dd, 1H), 7.40 (td, 1H), 7.66 (ddd, 1H), 7.83 (dd, 1H).

### Intermediate 397

### ethyl 5-fluoro-2-(trifluoromethoxy)benzoate

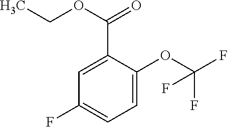 [SMILES: CCOC(=O)c1cc(F)ccc1OC(F)(F)F]

5-Fluoro-2-(trifluoromethoxy)benzoic acid (661 mg, 2.95 mmol) was solubilised in ethanol (6.6 mL) under argon, cooled to 0° C., thionyl dichloride (1.2 mL, 17 mmol) was added dropwise and the mixture was stirred for 6 h at reflux. The mixture was concentrated under reduced pressure to give 660 mg (89% yield) of the target compound, which was used without further purification.

¹H-NMR (400 MHz, DMSO-d₆): δ [ppm]=1.30 (t, 3H), 4.33 (q, 2H), 7.58-7.66 (m, 2H), 7.75 (ddd, 1H).

### Intermediate 398

### ethyl 4-methoxy-2-(trifluoromethoxy)benzoate

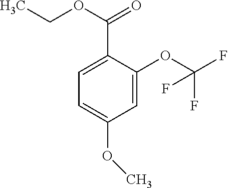 [SMILES: CCOC(=O)c1ccc(OC)cc1OC(F)(F)F]

4-Methoxy-2-(trifluoromethoxy)benzoic acid (100 mg, 423 μmol) was solubilised in ethanol (950 μL) under argon, cooled to 0° C., thionyl dichloride (180 μL, 2.4 mmol) was added dropwise and the mixture was stirred for 6 h at reflux. The mixture was concentrated under reduced pressure to give 110 mg (99% purity, 97% yield) of the target compound, which was used without further purification.

¹H-NMR (400 MHz, DMSO-d6): δ [ppm]=1.29 (t, 3H), 3.87 (s, 3H), 4.28 (q, 2H), 7.00-7.03 (m, 1H), 7.12 (dd, 1H), 7.94 (d, 1H).

### Intermediate 399

### ethyl 4-fluoro-2-(trifluoromethoxy)benzoate

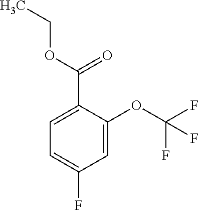 [SMILES: CCOC(=O)c1ccc(F)cc1OC(F)(F)F]

Thionyl dichloride (1.7 mL, 23 mmol) was added dropwise to 4-fluoro-2-(trifluoromethoxy)benzoic acid (900 mg, 4.02 mmol) under argon and it was stirred for 15 min at rt. The mixture was cooled to 0° C. and ethanol (9.0 mL) was added dropwise. The mixture was stirred for 2 h at 80° C. The mixture was evaporated, diluted with sat. sodium hydrogen carbonate solution and extracted three times with EtOAc. The combined organic layers were washed with water, dried and concentrated under reduced pressure to give 1.08 g of the target compound, which was used without further purification.

¹H-NMR (500 MHz, DMSO-d₆): δ [ppm]=1.31 (t, 3H), 4.32 (q, 2H), 7.46 (ddd, 1H), 7.53-7.56 (m, 1H), 8.04 (dd, 1H).

### Intermediate 400

### ethyl 4-bromo-2-(trifluoromethoxy)benzoate

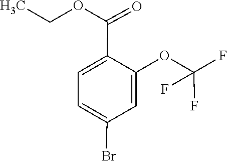 [SMILES: CCOC(=O)c1ccc(Br)cc1OC(F)(F)F]

4-Bromo-2-(trifluoromethoxy)benzoic acid (900 mg, 3.16 mmol) was solubilised in ethanol (7.1 mL) under argon, cooled to 0° C., thionyl dichloride (1.3 mL, 18 mmol) was added dropwise and the mixture was stirred for 2 h at reflux and overnight at rt. The mixture was concentrated under reduced pressure to give 1.14 g of the target compound, which was used without further purification.

¹H-NMR (400 MHz, DMSO-d6): δ [ppm]=1.30 (t, 3H), 4.32 (q, 2H), 7.81 (dd, 1H), 7.83-7.84 (m, 1H), 7.89 (d, 1H).

### Intermediate 403

### ethyl 2-(1,1,2,2-tetrafluoroethoxy)benzoate

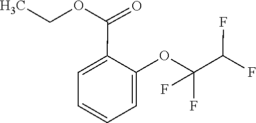 [SMILES: CCOC(=O)c1ccccc1OC(F)(F)C(F)F]

2-(1,1,2,2-Tetrafluoroethoxy)benzoic acid (500 mg, 2.10 mmol) was dissolved in ethanol (10 mL) under argon and cooled to 0° C. Thionyl dichloride (0.873 mL, 12 mmol) was added dropwise and the mixture was stirred for 5 min at 0° C., 6 h at reflux and overnight at rt. The mixture was concentrated under reduced pressure to give 544 mg (97% yield) of the title compound, which was used without further purification in the next step.

LC-MS (Method 2): Rₜ=1.27 min; MS (ESIpos): m/z=267 [M+H]⁺

¹H-NMR (400 MHz, DMSO-d6): δ [ppm]=1.29 (t, 3H), 4.30 (q, 2H), 6.77 (tt, 1H), 7.42-7.46 (m, 1H), 7.50 (td, 1H), 7.71 (ddd, 1H), 7.87 (dd, 1H).

### Intermediate 404

### 2-(dimethylamino)benzohydrazide

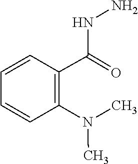

Methyl 2-(dimethylamino)benzoate (1.00 g, 5.58 mmol) was solubilised in propan-1-ol (12 mL), hydrazine hydrate (2.0 mL, 42 mmol) was added and the mixture was stirred for 48 h at 100° C. The mixture was concentrated under reduced pressure to give 1.00 g (94% purity, 94% yield) of the target compound, which was used without further purification.

LC-MS (Method 2): Rₜ=0.69 min; MS (ESIpos): m/z=180 [M+H]⁺

¹H-NMR (400 MHz, DMSO-d₆): δ [ppm]=2.70 (s, 6H), 4.48 (br s, 2H), 6.97 (td, 1H), 7.08 (dd, 1H), 7.34 (ddd, 1H), 7.46 (dd, 1H), 9.78 (s, 1H).

### Intermediate 405

### 2-(difluoromethoxy)benzohydrazide

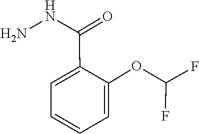 [SMILES: NNC(=O)c1ccccc1OC(F)F]

Ethyl 2-(difluoromethoxy)benzoate (488 mg, 2.26 mmol) was solubilised in ethanol (4.9 mL), hydrazine hydrate (550 μL, 11 mmol) was added and the mixture was stirred for 20 h at 90° C. The mixture was concentrated under reduced pressure to give 480 mg (90% purity, 95% yield) of the target compound, which was used without further purification.

LC-MS (Method 2): Rₜ=0.60 min; MS (ESIpos): m/z=203 [M+H]⁺

¹H-NMR (400 MHz, DMSO-d₆): δ [ppm]=4.50 (br s, 2H), 7.16 (t, 1H), 7.21-7.25 (m, 1H), 7.30 (td, 1H), 7.47 (dd, 1H), 7.51 (ddd, 1H), 9.45 (s, 1H).

### Intermediate 406

### 5-fluoropyridine-3-carbohydrazide

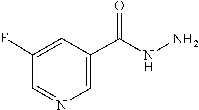 [SMILES: NNC(=O)c1cncc(F)c1]

Methyl 5-fluoropyridine-3-carboxylate (400 mg, 2.58 mmol) was solubilised in methanol (6.0 mL), hydrazine hydrate (630 μL, 13 mmol) was added and the mixture was stirred for 4 h at 60° C. The mixture was concentrated under reduced pressure to give 395 mg (100% purity, 99% yield) of the target compound, which was used without further purification.

¹H-NMR (400 MHz, DMSO-d₆): δ [ppm]=4.62 (br s, 2H), 8.04 (ddd, 1H), 8.73 (d, 1H), 8.85 (t, 1H), 10.06 (br s, 1H).

### Intermediate 407

### 5-chloropyridine-3-carbohydrazide

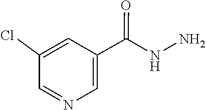 [SMILES: NNC(=O)c1cncc(Cl)c1]

Ethyl 5-chloropyridine-3-carboxylate (770 mg, 4.15 mmol) was solubilised in propan-1-ol (12 mL), hydrazine hydrate (1.5 mL, 31 mmol) was added and the mixture was stirred for 48 h at 100° C. The mixture was concentrated under reduced pressure to give 705 mg (98% purity, 97% yield) of the target compound, which was used without further purification.

LC-MS (Method 2): Rₜ=0.48 min; MS (ESIpos): m/z=172 [M+H]⁺

¹H-NMR (400 MHz, DMSO-d₆): δ [ppm]=4.63 (br s, 2H), 8.25 (dd, 1H), 8.77 (d, 1H), 8.91 (d, 1H), 10.06 (br s, 1H).

### Intermediate 408

### 2-(trifluoromethyl)pyridine-3-carbohydrazide

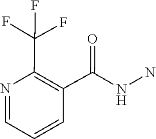

Ethyl 2-(trifluoromethyl)pyridine-3-carboxylate (1.90 g, 8.67 mmol) was solubilised in propan-1-ol (15 mL), hydrazine hydrate (3.2 mL, 65 mmol) was added and the mixture was stirred for 48 h at 100° C. The mixture was concentrated under reduced pressure to give 705 mg (98% purity, 97% yield) of the target compound, which was used without further purification.

¹H-NMR (400 MHz, DMSO-d₆): δ [ppm]=4.58 (br s, 2H), 7.77 (dd, 1H), 7.96 (dd, 1H), 8.80 (dd, 1H), 9.72 (s, 1H).

### Intermediate 409

### 5-fluoro-2-(trifluoromethoxy)benzohydrazide

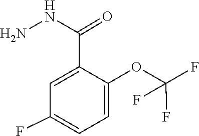 [SMILES: NNC(=O)c1cc(F)ccc1OC(F)(F)F]

Ethyl 5-fluoro-2-(trifluoromethoxy)benzoate (100 mg, 397 μmol) was solubilised in ethanol (870 μL), hydrazine hydrate (96 μL, 2.0 mmol) was added and the mixture was stirred for 20 h at 90° C. The mixture was concentrated under reduced pressure to give 95.0 mg (88% purity, 89% yield) of the target compound, which was used without further purification.

¹H-NMR (400 MHz, DMSO-d₆): δ [ppm]=4.54 (br s, 2H), 7.38 (dd, 1H), 7.45 (ddd, 1H), 7.48-7.54 (m, 1H), 9.68 (s, 1H).

### Intermediate 410

### 5-bromo-2-(trifluoromethoxy)benzohydrazide

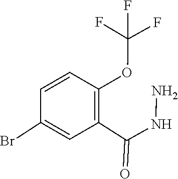 [SMILES: NNC(=O)c1cc(Br)ccc1OC(F)(F)F]

Ethyl 5-bromo-2-(trifluoromethoxy)benzoate (900 mg, 2.87 mmol) was solubilised in methanol (16 mL), hydrazine hydrate (1.4 mL, 29 mmol) was added and the mixture was stirred for 90 min in the microwave at 140° C. The reaction mixture was evaporated. The residue was partitioned between sat. ammonium chloride solution and EtOAc. The aqueous phase was extracted three times with EtOAc and the combined organic phases were washed with water, dried and concentrated under reduced pressure to give 770 mg (90% yield) of the target compound, which was used without further purification.

LC-MS (Method 2): Rₜ=0.86 min; MS (ESIpos): m/z=299 [M+H]⁺

¹H-NMR (400 MHz, DMSO-d₆): δ [ppm]=4.54 (br s, 2H), 7.42 (ddd, 1H), 7.69 (d, 1H), 7.78 (dd, 1H), 9.69 (br s, 1H).

### Intermediate 411

### 4-methoxy-2-(trifluoromethoxy)benzohydrazide

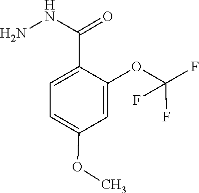 [SMILES: COc1ccc(C(=O)NN)c(OC(F)(F)F)c1]

Ethyl 4-methoxy-2-(trifluoromethoxy)benzoate (455 mg, 1.72 mmol) was solubilised in ethanol (9.1 mL), hydrazine hydrate (1.3 mL, 26 mmol) was added and the mixture was stirred for 88 h at 90° C. The mixture was concentrated under reduced pressure to give 455 mg (83% purity, 88% yield) of the target compound, which was used without further purification.

¹H-NMR (400 MHz, DMSO-d6): δ [ppm]=3.82 (s, 3H), 4.45 (br s, 2H), 6.92-6.95 (m, 1H), 7.03 (dd, 1H), 7.49 (d, 1H), 9.46 (br s, 1H).

### Intermediate 412

### 4-methoxy-2-(trifluoromethyl)benzohydrazide

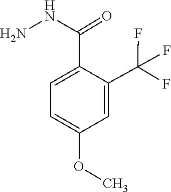 [SMILES: COc1ccc(C(=O)NN)c(C(F)(F)F)c1]

Methyl 4-methoxy-2-(trifluoromethyl)benzoate (400 mg, 1.71 mmol) was solubilised in ethanol (3.7 mL), hydrazine hydrate (830 μL, 17 mmol) was added and the mixture was stirred for 92 h at 90° C. The mixture was concentrated under reduced pressure to give 408 mg (66% purity, 67% yield) of the target compound, which was used without further purification.

LC-MS (Method 2): Rₜ=0.70 min; MS (ESIpos): m/z=235 [M+H]⁺

### Intermediate 413

### 4-fluoro-2-(trifluoromethoxy)benzohydrazide

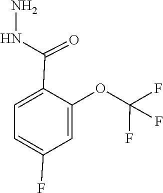 [SMILES: NNC(=O)c1ccc(F)cc1OC(F)(F)F]

Ethyl 4-fluoro-2-(trifluoromethoxy)benzoate (350 mg, 1.39 mmol) was solubilised in 2-methylbutan-2-ol (6.1 mL, 56 mmol), hydrazine hydrate (680 μL, 14 mmol) was added and the mixture was stirred for 24 h at 80° C. The mixture was concentrated under reduced pressure. Sat. ammonium chloride solution was added and it was extracted three times with EtOAc. The combined organic phases were washed with brine, dried and concentrated under reduced pressure to give 287 mg (87% yield) of the target compound, which was used without further purification.

¹H-NMR (400 MHz, DMSO-d₆): δ [ppm]=4.36 (s, 2H), 6.69-6.74 (m, 2H), 7.70 (d, 1H), 7.96 (s, 1H).

### Intermediate 414

### 4-bromo-2-(trifluoromethoxy)benzohydrazide

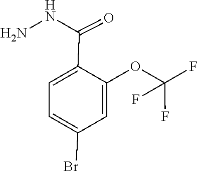 [SMILES: NNC(=O)c1ccc(Br)cc1OC(F)(F)F]

Ethyl 4-bromo-2-(trifluoromethoxy)benzoate (1.14 g, 87% purity, 3.17 mmol) was solubilised in ethanol (6.9 mL), hydrazine hydrate (770 μL, 16 mmol) was added and the mixture was stirred for 20 h at 90° C. The mixture was concentrated under reduced pressure to give 1.09 g (65% purity, 75% yield) of the target compound, which was used without further purification.

¹H-NMR (400 MHz, DMSO-d6): δ [ppm]=4.53 (br s, 2H), 7.48 (d, 1H), 7.68-7.73 (m, 2H), 9.65 (br s, 1H).

### Intermediate 415

### 4-chloro-2-(difluoromethoxy)benzohydrazide

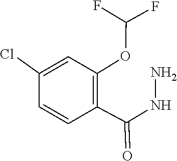 [SMILES: NNC(=O)c1ccc(Cl)cc1OC(F)F]

Methyl 4-chloro-2-(difluoromethoxy)benzoate (500 mg, 2.11 mmol) was solubilised in methanol (9.0 mL), hydrazine hydrate (510 μL, 11 mmol) was added and the mixture was stirred for 90 min in the microwave at 140° C. The reaction mixture was evaporated. The residue was partitioned between sat. ammonium chloride solution and EtOAc. The aqueous phase was extracted three times with EtOAc and the combined organic phases were washed with brine, dried and concentrated under reduced pressure to give 467 mg (93% yield) of the target compound, which was used without further purification.

LC-MS (Method 1): Rₜ=0.76 min; MS (ESIpos): m/z=237 [M+H]⁺

¹H-NMR (400 MHz, DMSO-d₆): δ [ppm]=4.55 (br s, 2H), 7.26 (t, 1H), 7.35-7.37 (m, 1H), 7.40 (dd, 1H), 7.49 (d, 1H), 9.49 (br s, 1H).

### Intermediate 418

### 4-cyanobenzohydrazide

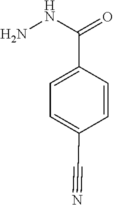 [SMILES: N#Cc1ccc(C(=O)NN)cc1]

Ethyl 4-cyanobenzoate (1.50 g, 8.56 mmol) was solubilised in ethanol (19 mL), hydrazine hydrate (2.1 mL, 43 mmol) was added and the mixture was stirred for 3 h at 90° C. The mixture was filtered, washed with EtOH and the solid was dried under reduced pressure to give 1.13 g (82% yield) of the target compound, which was used without further purification.

¹H-NMR (400 MHz, DMSO-d6): δ [ppm]=4.62 (br s, 2H), 7.92-7.99 (m, 4H), 10.05 (s, 1H).

### Intermediate 419

### 1,3-dimethyl-1H-pyrazole-4-carbohydrazide

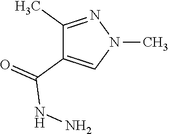

ethyl 1,3-dimethyl-1H-pyrazole-4-carboxylate (2.00 g, 11.9 mmol) was solubilised in ethanol (15 mL), hydrazine hydrate (4.8 mL, 60% purity, 59 mmol) was added and the mixture was stirred for 21 days at 110° C. After cooling to room temperature aqueous saturated ammonium chloride solution and ethyl acetate was added. After separation of the organic phase the aqueous phase was extracted two times with ethyl acetate. The combined organic phases were washed with brine, dried over sodium sulfate, filtrated over a hydrophobic phase separation filter paper and concentrated in vacuo. After drying we obtained 330 mg (80% purity, 14% yield) of the target compound, which was used without further purification.

LC-MS (Method 2): Rₜ=0.51 min; MS (ESIpos): m/z=155 [M+H]⁺

¹H-NMR (400 MHz, DMSO-d₆): δ [ppm]=2.29 (s, 3H), 3.34 (s, 1H), 3.74 (s, 3H, in water signal), 4.27 (br s, 2H), 7.97 (s, 1H), 9.06 (s, 1H).

### Intermediate 420

### 2-(1,1,2,2-tetrafluoroethoxy)benzohydrazide

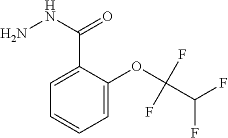 [SMILES: NNC(=O)c1ccccc1OC(F)(F)C(F)F]

Ethyl 2-(1,1,2,2-tetrafluoroethoxy)benzoate (539 mg, 2.02 mmol) was dissolved in ethanol (4.8 mL), hydrazine hydrate (990 μL, 20 mmol) was added and the reaction mixture was stirred under reflux for 23 h. The reaction mixture was allowed to cool down and concentrated under reduced pressure. The residue was treated twice with dichloromethane and concentrated under reduced pressure to afford 516 mg (92% purity, 93% yield) of the title compound which was used without further purification in the next step.

LC-MS (Method 2): Rₜ=0.76 min; MS (ESIpos): m/z=253 [M+H]⁺

¹H-NMR (400 MHz, DMSO-d6): δ [ppm]=4.45 (br s, 2H), 6.69 (tt, 1H), 7.36 (dd, 1H), 7.41 (td, 1H), 7.51 (dd, 1H), 7.55 (ddd, 1H), 9.48 (s, 1H).

### Intermediate 421

### 4-bromo-2-(difluoromethoxy)benzohydrazide

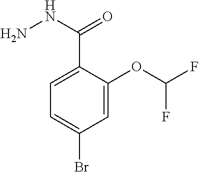 [SMILES: NNC(=O)c1ccc(Br)cc1OC(F)F]

Methyl 4-bromo-2-(difluoromethoxy)benzoate (445 mg, 1.58 mmol) was dissolved in methanol (4.4 mL), hydrazine hydrate (770 μL, 16 mmol) was added and the reaction mixture was stirred under reflux for 4 h. The reaction mixture was allowed to cool down and concentrated under reduced pressure. The residue was treated three times with dichloromethane and concentrated under reduced pressure to afford 460 mg (93% purity, 96% yield) of the title compound which was used without further purification in the next step.

LC-MS (Method 2): Rₜ=0.81 min; MS (ESIpos): m/z=281 [M+H]⁺

1H-NMR (400 MHz, DMSO-d6): δ [ppm]=4.52 (br s, 2H), 7.26 (t, 1H), 7.42 (d, 1H), 7.47-7.49 (m, 1H), 7.53 (dd, 1H), 9.49 (s, 1H).

### Intermediate 422

### ethyl [2-cyano-4-(trifluoromethyl)phenyl]carbamate

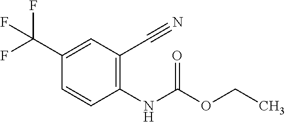 [SMILES: CCOC(=O)Nc1ccc(C(F)(F)F)cc1C#N]

2-Amino-5-(trifluoromethyl)benzonitrile (300 mg, 1.61 mmol) was stirred in ethyl carbonochloridate (6.4 mL) for 8 h at reflux. The mixture was concentrated under reduced pressure to give 410 mg (100% purity, 99% yield) of the target compound, which was used without further purification.

LC-MS (Method 2): Rₜ=1.15 min; MS (ESIneg): m/z=257 [M−H]⁻.

¹H-NMR (400 MHz, DMSO-d6): δ [ppm]=1.27 (t, 3H), 4.19 (q, 2H), 7.81 (d, 1H), 8.03 (dd, 1H), 8.29 (d, 1H), 10.14 (s, 1H).

### Intermediate 423

### ethyl [2-cyano-6-(propan-2-yl)phenyl]carbamate

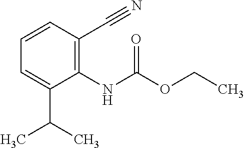 [SMILES: CCOC(=O)Nc1c(C#N)cccc1C(C)C]

Ethyl (2-bromo-6-cyanophenyl)carbamate (1.00 g, 3.72 mmol), lithium hydroxide (712 mg, 29.7 mmol) and (4,4′-di-tert-butyl-2,2′-bipyridine-kappa²N¹,N¹)(bis{3,5-difluoro-2-[5-(trifluoromethyl)pyridin-2-yl-kappaN]phenyl-kappaC¹})iridium(1+) hexafluorophosphate (83.4 mg, 74.3 μmol) were dissolved in a reaction vial in (trifluoromethyl)benzene (70 mL). In a separate vial, 1,2-dimethoxyethane-dichloronickel (1:1) (40.8 mg, 186 μmol) and 4,4′-di-tert-butyl-2,2′-bipyridine (49.9 mg, 186 μmol) were stirred in N,N-dimethylacetamide (25 mL, 270 mmol) for 5 min. The catalyst solution was added to the sealed reaction vial. The mixture was degassed by sparging with argon for 15 min. Then 2-bromopropane (2.4 mL, 26 mmol) and 1,1,1,3,3,3-hexamethyl-2-(trimethylsilyl)trisilane (1.1 mL, 3.7 mmol) were added. The vial was stirred in a water bath and irradiated by two 40 W Kessil LED Aquarium lamps (A160WE tuna blue) for 24 h. The mixture was diluted with water and extracted three times with EtOAc. The combined organic layers were dried and concentrated under reduced pressure. The residue was purified by flash chromatography to give 530 mg (98% purity, 60% yield) of the target compound.

LC-MS (Method 2): Rₜ=1.06 min; MS (ESIpos): m/z=233 [M+H]⁺

¹H-NMR (400 MHz, DMSO-d₆): δ [ppm]=1.14 (d, 6H), 1.17-1.30 (m, 3H), 3.12-3.23 (m, 1H), 4.06-4.17 (m, 2H), 7.42-7.47 (m, 1H), 7.68 (d, 2H), 9.38 (br m, 1H).

### Intermediate 424

### di-tert-butyl [2-cyano-6-(trifluoromethyl)phenyl]-2-imidodicarbonate

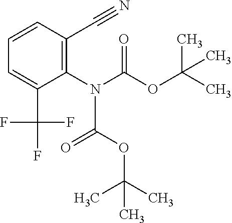 [SMILES: CC(C)(C)OC(=O)N(C(=O)OC(C)(C)C)c1c(C#N)cccc1C(F)(F)F]

To a solution of 2-amino-3-(trifluoromethyl)benzonitrile (2.00 g, 10.7 mmol) in dioxane (48 ml) was added gelöst, mit N,N-diisopropylethylamine (4.7 ml, 27 mmol; CAS-RN:[7087-68-5]), 4-(N,N-dimethylamino)pyridine (4.7 ml, 27 mmol; CAS-RN:[7087-68-5]) and di-tert-butyl dicarbonate (6.2 ml, 27 mmol; CAS-RN:[24424-99-5]). This reaction mixture was stirred for 90 h at room temperature, then concentrated under vacuum The resulting residue was purified via a Biotage chromatography system (50 g SiO2 SNAP Ultra-column, hexane/0-80% ethyl acetate) to obtain 3.89 g (56% yield) of the desired title compound.

¹H-NMR (400 MHz, DMSO-d₆): δ [ppm]=1.33 (s, 18H), 7.85 (t, 1H), 8.19 (dd, 1H), 8.34 (dd, 1H).

### Intermediate 425

### 2-(1-methyl-1H-pyrazol-3-yl)[1,2,4]triazolo[1,5-c]quinazolin-5(6H)-one

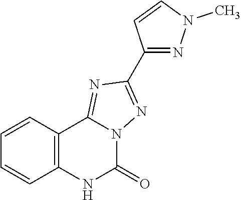 [SMILES: Cn1ccc(-c2nc3c4ccccc4[nH]c(=O)n3n2)n1]

Methyl (2-cyanophenyl)carbamate (503 mg, 2.86 mmol) and 1-methyl-1H-pyrazole-3-carbohydrazide (400 mg, 2.86 mmol) were stirred in DMF (10 mL) at 120° C. for 20 h. Water was added to the mixture, filtered, washed with water and the solid was dried under reduced pressure at 60° C. to give 630 mg (83% yield) of the target compound, which was used without further purification.

LC-MS (Method 2): Rₜ=0.48 min; MS (ESIpos): m/z=267 [M+H]+

The following intermediates were prepared similarly to intermediate 425:

| | Structure |
|---|---|
| | IUPAC-Name |
| | LC-MS (method): Retention time; Mass found |
| Intermediate | ¹H-NMR |
| Intermediate 426 | 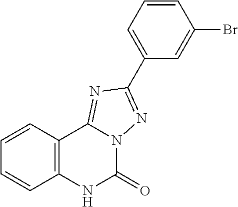 [SMILES: O=c1[nH]c2ccccc2c2nc(-c3cccc(Br)c3)nn12] |
| | 2-(3-bromophenyl)[1,2,4]triazolo |
| | [1,5-c]quinazolin-5(6H)-one |
| | LC-MS (Method 2): Rt = 0.76 min; |
| | MS (ESIpos): m/z = 341 [M + H]⁺ |
| | ¹H-NMR (400 MHz, DMSO-d₆): δ [ppm] = |
| | 7.39-7.48 (m, 2H), 7.56 (t, 1H), 7.70-7.79 |
| | (m, 2H), 8.24 (t, 2H), 8.34 (s, 1H), 12.38 |
| | (br s, 1H). |
| Intermediate 427 | 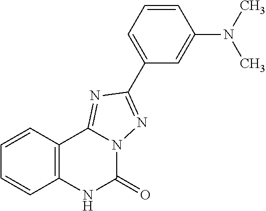 [SMILES: CN(C)c1cccc(-c2nc3c4ccccc4[nH]c(=O)n3n2)c1] |
| | 2-[3-(dimethylamino)phenyl][1,2,4] |
| | triazolo[1,5-c]quinazolin-5(6H)-one |
| | LC-MS (Method 2): Rt = 0.71 min; |
| | MS (ESIpos): m/z = 306 [M + H]⁺ |
| | ¹H-NMR (400 MHz, DMSO-d₆): δ [ppm] = |
| | 3.01 (s, 6H), 6.88-6.94 (m, 1H), 7.34-7.47 |
| | (m, 3H), 7.52-7.56 (m, 2H), 7.69-7.74 (m, |
| | 1H), 8.24 (dd, 1H), 12.32 (br s, 1H). |
| Intermediate 428 | 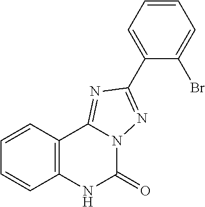 [SMILES: O=c1[nH]c2ccccc2c2nc(-c3ccccc3Br)nn12] |
| | 2-(2-bromophenyl)[1,2,4]triazolo |
| | [1,5-c]quinazolin-5(6H)-one |
| | LC-MS (Method 1): Rt = 1.04 min; |
| | MS (ESIpos): m/z = 341 [M + H]⁺ |
| | ¹H-NMR (400 MHz, DMSO-d₆): δ [ppm] = |
| | 7.39-7.52 (m, 3H), 7.58 (td, 1H), 7.73 |
| | (ddd, 1H), 7.85 (dd, 1H), 7.92 (dd, 1H), |
| | 8.21 (dd, 1H), 12.42 (br s, 1H). |
| Intermediate 429 | 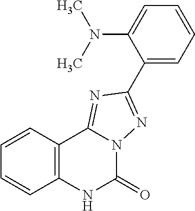 [SMILES: CN(C)c1ccccc1-c1nc2c3ccccc3[nH]c(=O)n2n1] |
| | 2-[2-(dimethylamino)phenyl][1,2,4] |
| | triazolo[1,5-c]quinazolin-5(6H)-one |
| | LC-MS (Method 2): Rt = 0.67 min; |
| | MS (ESIpos): m/z = 306 [M + H]⁺ |
| | ¹H-NMR (400 MHz, DMSO-d₆): δ [ppm] = |
| | 2.66 (s, 6H), 7.00 (td, 1H), 7.11 (d, 1H), |
| | 7.38-7.43 (m, 2H), 7.46 (d, 1H), 7.67 (dd, 1H), |
| | 7.71 (ddd, 1H), 8.21 (dd, 1H), 12.33 (br s, 1H). |
| Intermediate 430 | 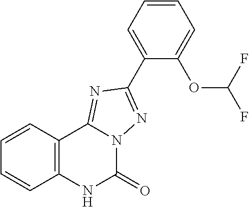 [SMILES: O=c1[nH]c2ccccc2c2nc(-c3ccccc3OC(F)F)nn12] |
| | 2-[2-(difluoromethoxy)phenyl][1,2,4] |
| | triazolo[1,5-c]quinazolin-5(6H)-one |
| | LC-MS (Method 2): Rt = 0.73 min; |
| | MS (ESIpos): m/z = 329 [M + H]⁺ |
| | ¹H-NMR (400 MHz, DMSO-d₆): δ [ppm] = |
| | 7.28 (t, 1H), 7.38-7.51 (m, 4H), 7.60-7.66 |
| | (m, 1H), 7.70-7.75 (m, 1H), 8.15 (dd, 1H), |
| | 8.21 (dd, 1H), 12.42 (brs, 1H). |
| Intermediate 431 | 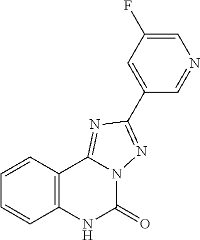 [SMILES: O=c1[nH]c2ccccc2c2nc(-c3cncc(F)c3)nn12] |
| | 2-(5-fluoropyridin-3-yl)[1,2,4]triazolo |
| | [1,5-c]quinazolin-5(6H)-one |
| | LC-MS (Method 2): Rt = 0.61 min; |
| | MS (ESIpos): m/z = 282 [M + H]⁺ |
| | ¹H-NMR (400 MHz, DMSO-d₆): δ [ppm] = |
| | 7.41-7.49 (m, 2H), 7.71-7.77 (m, 1H), 8.24 |
| | (dd, 1H), 8.36 (ddd, 1H), 8.78 (d, 1H), 9.25 |
| | (t, 1H), 12.45 (br s, 1H). |
| Intermediate 432 | 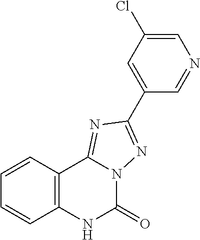 [SMILES: O=c1[nH]c2ccccc2c2nc(-c3cncc(Cl)c3)nn12] |
| | 2-(5-chloropyridin-3-yl)[1,2,4]triazolo |
| | [1,5-c]quinazolin-5(6H)-one |
| | LC-MS (Method 2): Rt = 0.62 min; |
| | MS (ESIpos): m/z = 298 [M + H]⁺ |
| | ¹H-NMR (400 MHz, DMSO-d₆): δ [ppm] = |
| | 7.41-7.48 (m, 2H), 7.74 (ddd, 1H), 8.25 |
| | (dd, 1H), 8.56 (t, 1H), 8.83 (d, 1H), 9.30 |
| | (d, 1H), 12.45 (br s, 1H). |
| Intermediate 433 | 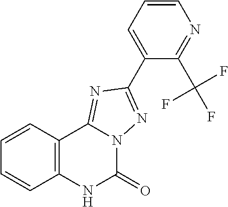 [SMILES: O=c1[nH]c2ccccc2c2nc(-c3cccnc3C(F)(F)F)nn12] |
| | 2-[2-(trifluoromethyl)pyridin-3-yl][1,2,4] |
| | triazolo[1,5-c]quinazolin-5(6H)-one |
| | LC-MS (Method 2): Rt = 0.57 min; |
| | MS (ESIpos): m/z = 332 [M + H]⁺ |
| | ¹H-NMR (400 MHz, DMSO-d₆): δ [ppm] = |
| | 7.41-7.46 (m, 1H), 7.48 (d, 1H), 7.75 (ddd, |
| | 1H), 7.94 (dd, 1H), 8.20 (dd, 1H), 8.43 |
| | (dd, 1H), 8.95 (dd, 1H), 12.51 (br s, 1H). |
| Intermediate 434 | 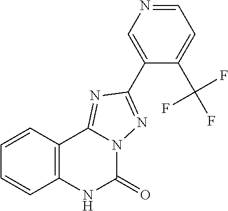 [SMILES: O=c1[nH]c2ccccc2c2nc(-c3cnccc3C(F)(F)F)nn12] |
| | 2-[4-(trifluoromethyl)pyridin-3-yl][1,2,4] |
| | triazolo[1,5-c]quinazolin-5(6H)-one |
| | LC-MS (Method 2): Rt = 0.60 min; |
| | MS (ESIpos): m/z = 332 [M + H]⁺ |
| | ¹H-NMR (400 MHz, DMSO-d₆): δ [ppm] = |
| | 7.40-7.45 (m, 1H), 7.48 (d, 1H), 7.74 |
| | (ddd, 1H), 8.01 (d, 1H), 8.20 (dd, 1H), 9.05 |
| | (d, 1H), 9.23 (s, 1H). |
| Intermediate 435 | 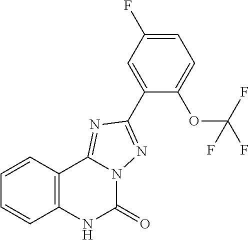 [SMILES: O=c1[nH]c2ccccc2c2nc(-c3cc(F)ccc3OC(F)(F)F)nn12] |
| | 2-[5-fluoro-2-(trifluoromethoxy)phenyl][1,2,4] |
| | triazolo[1,5-c]quinazolin-5(6H)-one |
| | LC-MS (Method 2): Rt = 0.79 min; |
| | MS (ESIpos): m/z = 365 [M + H]⁺ |
| | ¹H-NMR (400 MHz, DMSO-d₆): δ [ppm] = |
| | 7.41-7.49 (m, 2H), 7.54-7.61 (m, 1H), 7.66- |
| | 7.76 (m, 2H), 8.04 (dd, 1H), 8.20 (d, 1H), |
| | 12.45 (br s, 1H). |
| Intermediate 436 | 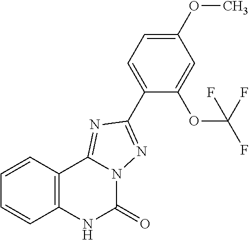 [SMILES: COc1ccc(-c2nc3c4ccccc4[nH]c(=O)n3n2)c(OC(F)(F)F)c1] |
| | 2-[4-methoxy-2-(trifluoromethoxy)phenyl] |
| | [1,2,4]triazolo[1,5-c]quinazolin-5(6H)-one |
| | LC-MS (Method 2): Rt = 0.81 min; |
| | MS (ESIpos): m/z = 377 [M + H]⁺ |
| | ¹H-NMR (400 MHz, DMSO-d₆): δ [ppm] = |
| | 3.90 (s, 3H), 7.08-7.12 (m, 1H), 7.22 (dd, 1H), |
| | 7.40-7.47 (m, 2H), 7.72 (ddd, 1H), 8.19 (dd, |
| | 1H), 8.23 (d, 1H), 12.37 (br s, 1H). |
| Intermediate 437 | 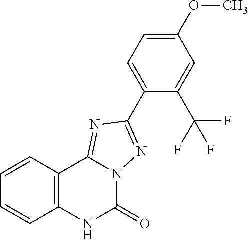 [SMILES: COc1ccc(-c2nc3c4ccccc4[nH]c(=O)n3n2)c(C(F)(F)F)c1] |
| | 2-[4-methoxy-2-(trifluoromethyl)phenyl] |
| | [1,2,4]triazolo[1,5-c]quinazolin-5(6H)-one |
| | LC-MS (Method 2): Rt = 0.74 min; |
| | MS (ESIpos): m/z = 361 [M + H]⁺ |
| | ¹H-NMR (400 MHz, DMSO-d₆): δ [ppm] = |
| | 3.93 (s, 3H), 7.38-7.44 (m, 3H), 7.46 (d, 1H), |
| | 7.72 (ddd, 1H), 7.93 (d, 1H), 8.17 (dd, 1H), |
| | 12.34 (br s, 1H). |
| Intermediate 438 | 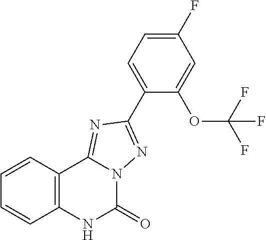 [SMILES: O=c1[nH]c2ccccc2c2nc(-c3ccc(F)cc3OC(F)(F)F)nn12] |
| | 2-[4-fluoro-2-(trifluoromethoxy)phenyl] |
| | [1,2,4]triazolo[1,5-c]quinazolin-5(6H)-one |
| | LC-MS (Method 2): Rt = 0.81 min; |
| | MS (ESIpos): m/z = 365 [M + H]⁺ |
| Intermediate 439 | 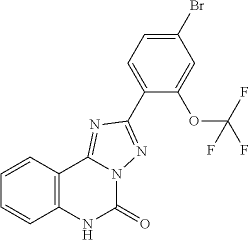 [SMILES: O=c1[nH]c2ccccc2c2nc(-c3ccc(Br)cc3OC(F)(F)F)nn12] |
| | 2-[4-bromo-2-(trifluoromethoxy)phenyl] |
| | [1,2,4]triazolo[1,5-c]quinazolin-5(6H)-one |
| | LC-MS (Method 2): Rt = 0.91 min; |
| | MS (ESIpos): m/z = 425 [M + H]⁺ |
| | ¹H-NMR (400 MHz, DMSO-d₆): δ [ppm] = |
| | 7.40-7.48 (m, 2H), 7.73 (ddd, 1H), 7.86-7.90 |
| | (m, 2H), 8.18-8.21 (m, 1H), 8.25 (d, 1H), |
| | 12.43 (s, 1H). |
| Intermediate 440 | 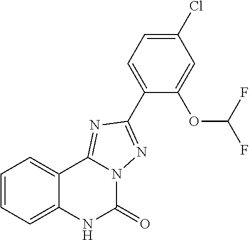 [SMILES: O=c1[nH]c2ccccc2c2nc(-c3ccc(Cl)cc3OC(F)F)nn12] |
| | 2-[4-chloro-2-(difluoromethoxy)phenyl] |
| | [1,2,4]triazolo[1,5-c]quinazolin-5(6H)-one |
| | LC-MS (Method 1): Rt = 1.20 min; |
| | MS (ESIpos): m/z = 363 [M + H]⁺ |
| | ¹H-NMR (400 MHz, DMSO-d₆): δ [ppm] = |
| | 7.37 (t, 1H), 7.40-7.48 (m, 2H), 7.54 (d, 1H), |
| | 7.58 (dd, 1H), 7.73 (ddd, 1H), 8.17-8.22 |
| | (m, 2H), 12.41 (br s, 1H). |
| Intermediate 442 | 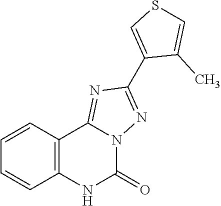 [SMILES: Cc1cscc1-c1nc2c3ccccc3[nH]c(=O)n2n1] |
| | 2-(4-methylthiophen-3-yl)[1,2,4]triazolo |
| | [1,5-c]quinazolin-5(6H)-one |
| | LC-MS (Method 1): Rt = 1.07 min; |
| | MS (ESIpos): m/z = 283 [M + H]⁺ |
| | ¹H-NMR (400 MHz, DMSO-d₆): δ [ppm] = |
| | 2.60 (d, 3H), 7.36-7.38 (m, 1H), 7.71 |
| | (ddd, 1H), 8.20 (ddd, 1H), 8.27 (d, 1H), 7.41 |
| | (ddd, 1H), 7.45 (d, 1H), 12.32 (br s, 1H). |
| Intermediate 443 | 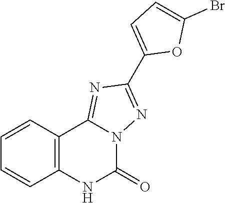 [SMILES: O=c1[nH]c2ccccc2c2nc(-c3ccc(Br)o3)nn12] |
| | 2-(5-bromofuran-2-yl)[1,2,4]triazolo |
| | [1,5-c]quinazolin-5(6H)-one |
| | LC-MS (Method 2): Rₜ = 0.60 min; |
| | MS (ESIpos): m/z = 331 [M + H]⁺ |
| | ¹H-NMR (400 MHz, DMSO-d₆): δ [ppm] = |
| | 6.87 (d, 1H), 7.30 (d, 1H), 7.38-7.49 (m, 2H), |
| | 7.68-7.77 (m, 1H), 8.19 (dd, 1H), 12.38 |
| | (br s, 1H). |
| Intermediate 444 | 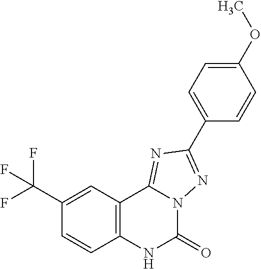 [SMILES: COc1ccc(-c2nc3c4cc(C(F)(F)F)ccc4[nH]c(=O)n3n2)cc1] |
| | 2-(4-methoxyphenyl)-9-(trifluoromethyl) |
| | [1,2,4]triazolo[1,5-c]quinazolin-5(6H)-one |
| | LC-MS (Method 2): Rₜ = 0.77 min; |
| | MS (ESIneg): m/z = 359 [M − H]⁻ |
| | ¹H-NMR (400 MHz, DMSO-d₆): δ [ppm] = |
| | 3.85 (s, 3H), 7.10-7.15 (m, 2H), 7.61 (d, 1H), |
| | 8.04 (dd, 1H), 8.14-8.21 (m, 2H), 8.43 (d, 1H), |
| | 12.64 (br s, 1H). |
| Intermediate 445 | 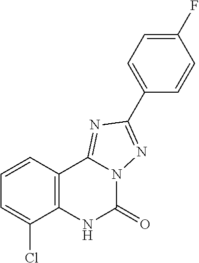 [SMILES: O=c1[nH]c2c(Cl)cccc2c2nc(-c3ccc(F)cc3)nn12] |
| | 7-chloro-2-(4-fluorophenyl)[1,2,4]triazolo |
| | [1,5-c]quinazolin-5(6H)-one |
| | LC-MS (Method 2): Rₜ = 0.68 min; |
| | MS (ESIpos): m/z = 315 [M + H]⁺ |
| Intermediate 446 | 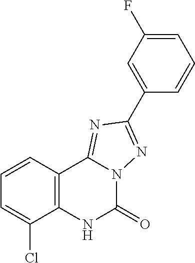 [SMILES: O=c1[nH]c2c(Cl)cccc2c2nc(-c3cccc(F)c3)nn12] |
| | 7-chloro-2-(3-fluorophenyl)[1,2,4]triazolo |
| | [1,5-c]quinazolin-5(6H)-one |
| | LC-MS (Method 2): Rₜ = 0.68 min; |
| | MS (ESIpos): m/z = 315 [M + H]⁺ |
| Intermediate 447 | 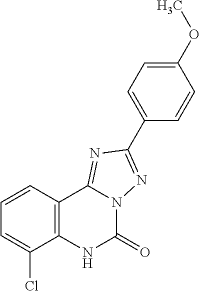 [SMILES: COc1ccc(-c2nc3c4cccc(Cl)c4[nH]c(=O)n3n2)cc1] |
| | 7-chloro-2-(4-methoxyphenyl)[1,2,4]triazolo |
| | [1,5-c]quinazolin-5(6H)-one |
| | LC-MS (Method 2): Rₜ = 0.64 min; |
| | MS (ESIpos): m/z = 327 [M + H]⁺ |
| | ¹H-NMR (400 MHz, DMSO-d₆): δ [ppm] = |
| | 3.85 (s, 3H), 7.10-7.17 (m, 2H), 7.31-7.53 |
| | (m, 1H), 7.41 (t, 1H), 7.81-7.87 (m, 1H), |
| | 8.13-8.19 (m, 2H), 8.19-8.27 (m, 1H), |
| | 11.83 (s, 1H). |
| Intermediate 448 | 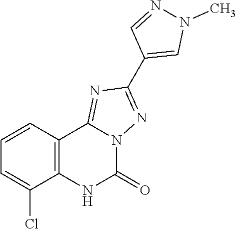 [SMILES: Cn1cc(-c2nc3c4cccc(Cl)c4[nH]c(=O)n3n2)cn1] |
| | 7-chloro-2-(1-methyl-1H-pyrazol-4-yl)[1,2,4] |
| | triazolo[1,5-c]quinazolin-5(6H)-one |
| | LC-MS (Method 2): Rₜ = 0.47 min; |
| | MS (ESIpos): m/z = 301 [M + H]⁺ |
| Intermediate 449 | 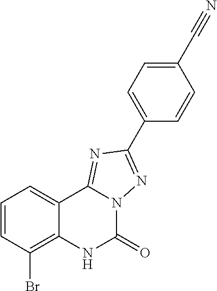 [SMILES: N#Cc1ccc(-c2nc3c4cccc(Br)c4[nH]c(=O)n3n2)cc1] |
| | 4-(7-bromo-5-oxo-5,6-dihydro[1,2,4]triazolo |
| | [1,5-c]quinazolin-2-yl)benzonitrile |
| | LC-MS (Method 2): Rt = 0.67 min; |
| | MS (ESIpos): m/z = 366 [M + H]⁺ |
| | ¹H-NMR (400 MHz, DMSO-d₆): δ [ppm] = |
| | 7.35 (t, 1H), 8.02 (dd, 1H), 8.06 (d, 2H), 8.27 |
| | (dd, 1H), 8.39 (d, 2H), 11.51 (br s, 1H). |
| Intermediate 450 | 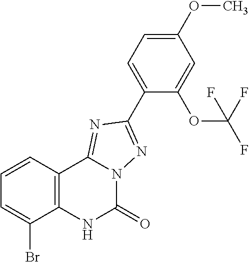 [SMILES: COc1ccc(-c2nc3c4cccc(Br)c4[nH]c(=O)n3n2)c(OC(F)(F)F)c1] |
| | 7-bromo-2-[4-methoxy-2-(trifluoromethoxy) |
| | phenyl][1,2,4]triazolo[1,5-c]quinazolin- |
| | 5(6H)-one |
| | LC-MS (Method 2): Rt = 0.76 min; |
| | MS (ESIpos): m/z = 455 [M + H]⁺ |
| | ¹H-NMR (400 MHz, DMSO-d₆): δ [ppm] = |
| | 3.90 (s, 3H), 7.10 (d, 1H), 7.22 (dd, 1H), 7.35 |
| | (t, 1H), 8.01 (dd, 1H), 8.20-8.25 (m, 2H), |
| | 11.44 (br s, 1H). |
| Intermediate 451 | 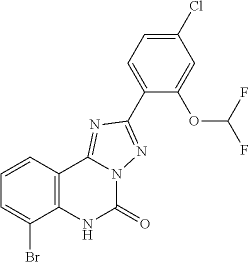 [SMILES: O=c1[nH]c2c(Br)cccc2c2nc(-c3ccc(Cl)cc3OC(F)F)nn12] |
| | 7-bromo-2-[4-chloro-2-(difluoromethoxy) |
| | phenyl][1,2,4]triazolo[1,5-c]quinazolin- |
| | 5(6H)-one |
| | LC-MS (Method 2): Rt = 0.82 min; |
| | MS (ESIpos): m/z = 441 [M + H]⁺ |
| | ¹H-NMR (400 MHz, DMSO-d₆): δ [ppm] = |
| | 7.35 (t, 1H), 7.38 (t, 1H), 7.53-7.56 (m, 1H), |
| | 7.58 (dd, 1H), 8.02 (dd, 1H), 8.20 (d, 1H), 8.24 |
| | (dd, 1H), 11.49 (br s, 1H). |
| Intermediate 452 | 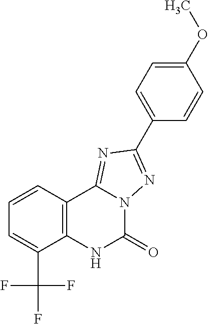 [SMILES: COc1ccc(-c2nc3c4cccc(C(F)(F)F)c4[nH]c(=O)n3n2)cc1] |
| | 2-(4-methoxyphenyl)-7-(trifluoromethyl) |
| | [1,2,4]triazolo[1,5-c]quinazolin-5(6H)-one |
| | LC-MS (Method 2): Rₜ = 0.72 min; |
| | MS (ESIneg): m/z = 359 [M − H]⁻ |
| Intermediate 453 | 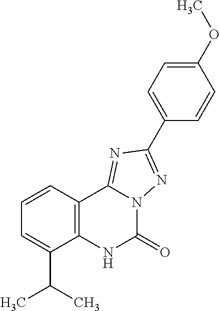 [SMILES: COc1ccc(-c2nc3c4cccc(C(C)C)c4[nH]c(=O)n3n2)cc1] |
| | 2-(4-methoxyphenyl)-7-(propan-2-yl) |
| | [1,2,4]triazolo[1,5-c]quinazolin-5(6H)-one |
| | LC-MS (Method 2): Rt = 0.82 min; |
| | MS (ESIpos): m/z = 335 [M + H]⁺ |
| Intermediate 454 | 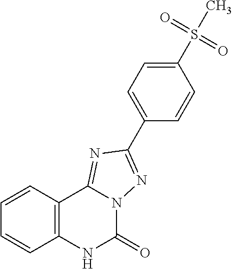 [SMILES: CS(=O)(=O)c1ccc(-c2nc3c4ccccc4[nH]c(=O)n3n2)cc1] |
| | 2-[4-(methanesulfonyl)phenyl][1,2,4]triazolo |
| | [1,5-c]quinazolin-5(6H)-one |
| | LC-MS (Method 2): Rₜ = 0.58 min; |
| | MS (ESIpos): m/z = 341 [M + H]⁺ |
| Intermediate 455 | 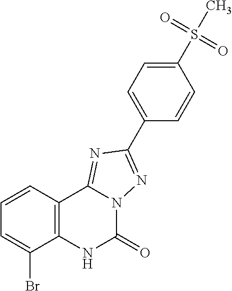 [SMILES: CS(=O)(=O)c1ccc(-c2nc3c4cccc(Br)c4[nH]c(=O)n3n2)cc1] |
| | 7-bromo-2-[4-(methanesulfonyl)phenyl] |
| | [1,2,4]triazolo[1,5-c]quinazolin-5(6H)-one |
| | LC-MS (method 2): Rₜ = 0.64 min; |
| | MS (ESIpos): m/z = 419 [M]⁺ |
| Intermediate 456 | 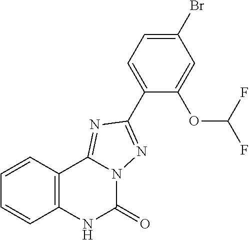 [SMILES: O=c1[nH]c2ccccc2c2nc(-c3ccc(Br)cc3OC(F)F)nn12] |
| | 2-[4-bromo-2-(difluoromethoxy)phenyl] |
| | [1,2,4]triazolo[1,5-c]quinazolin-5(6H)-one |
| | LC-MS (Method 2): Rₜ = 0.81 min; |
| | MS (ESIpos): m/z = 407 [M + H]⁺ |
| | ¹H-NMR (400 MHz, DMSO-d₆): δ [ppm] = |
| | 7.37 (t, 1H), 7.40-7.49 (m, 2H), 7.65 (d, 1H), |
| | 7.69-7.76 (m, 2H), 8.12 (d, 1H), 8.20 |
| | (dd, 1H), 12.41 (s, 1H). |
| Intermediate 457 | 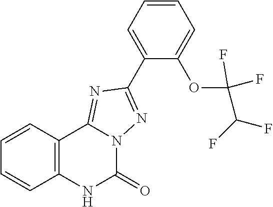 [SMILES: O=c1[nH]c2ccccc2c2nc(-c3ccccc3OC(F)(F)C(F)F)nn12] |
| | 2-[2-(1,1,2,2-tetrafluoroethoxy)phenyl][1,2,4] |
| | triazolo[1,5-c]quinazolin-5(6H)-one |
| | LC-MS (Method 2): Rₜ = 0.85 min; |
| | MS (ESIpos): m/z = 379 [M + H]⁺ |
| | ¹H-NMR (400 MHz, DMSO-d₆): δ [ppm] = |
| | 7.06 (tt, 1H), 7.42-7.46 (m, 1H), 7.49 (d, 1H), |
| | 7.55 (d, 1H), 7.61 (td, 1H), 7.68 (td, 1H), 7.74 |
| | (ddd, 1H), 8.24 (dd, 1H), 8.35 (dd, 1H), 12.45 |
| | (br s, 1H). |

### Intermediate 458

### 2-[2-(methanesulfonyl)phenyl][1,2,4]triazolo[1,5-c]quinazolin-5(6H)-one

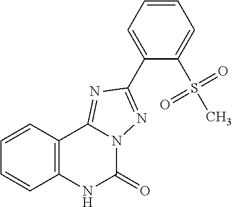 [SMILES: CS(=O)(=O)c1ccccc1-c1nc2c3ccccc3[nH]c(=O)n2n1]

### Step 1:

Methyl (2-cyanophenyl)carbamate (206 mg, 1.17 mmol) and 2-(methanesulfonyl)benzohydrazide (300 mg, 1.40 mmol) were stirred in DMF (8.0 mL) for 48 h at 120° C. The mixture was cooled down, poured into water, concentrated on the rotavap, water added, filtered, washed with water and the solid was dried under reduced pressure at 50° C.

A small sample was purified by HPLC yielding 20 mg of pure product.

¹H-NMR (400 MHz, DMSO-d₆): δ [ppm]=3.73 (s, 3H), 7.30-7.35 (m, 1H), 7.43 (d, 1H), 7.66 (ddd, 1H), 7.85 (ddd, 1H), 7.88-7.94 (m, 2H), 8.13-8.19 (m, 2H).

### Step 2:

The solid (mixture of target compound and intermediate) was solubilised in 1,2-dichloroethane (3.0 mL), TFA (230 μL, 2.9 mmol) was added and the mixture was stirred for 7 h at 60° C. TFA (230 μL, 2.9 mmol) was added and the mixture was stirred for 48 h at 90° C. The mixture was evaporated, diluted with water, filtered, washed with water and the solid was dried under reduced pressure at 45° C. to give 128 mg (95% purity, 61% yield) of the target compound, which was used without further purification.

LC-MS (Method 2): Rₜ=0.57 min; MS (ESIpos): m/z=341 [M+H]⁺

### Intermediate 459

### 7-bromo-2-[4-chloro-2-(trifluoromethoxy)phenyl][1,2,4]triazolo[1,5-c]quinazolin-5(6H)-one

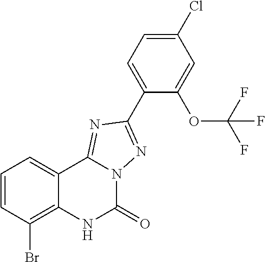 [SMILES: O=c1[nH]c2c(Br)cccc2c2nc(-c3ccc(Cl)cc3OC(F)(F)F)nn12]

Methyl (2-bromo-6-cyanophenyl)carbamate (361 mg, 1.41 mmol) and 4-chloro-2-(trifluoromethoxy)benzohydrazide (360 mg, 1.41 mmol) were stirred in DMF (8.0 mL) for 6 h at 120° C. TFA (1.1 mL, 14 mmol) was added and the mixture was stirred for 20 min at 120° C. Water was added to the mixture, filtered, washed with water and the solid was dried under reduced pressure at 60° C. to give 470 mg (72% yield) of the target compound, which was used without further purification.

LC-MS (Method 2): Rₜ=0.77 min; MS (ESIpos): m/z=459 [M+H]⁺

¹H-NMR (400 MHz, DMSO-d₆): δ [ppm]=7.36 (t, 1H), 7.75 (dd, 1H), 7.78 (d, 1H), 8.03 (dd, 1H), 8.23 (dd, 1H), 8.33 (d, 1H), 11.51 (br s, 1H).

### Intermediate 460

### 2-[5-bromo-2-(trifluoromethoxy)phenyl][1,2,4]triazolo[1,5-c]quinazolin-5(6H)-one

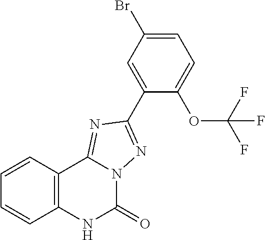 [SMILES: O=c1[nH]c2ccccc2c2nc(-c3cc(Br)ccc3OC(F)(F)F)nn12]

Methyl (2-cyanophenyl)carbamate (236 mg, 1.34 mmol) and 5-bromo-2-(trifluoromethoxy)benzohydrazide (400 mg, 1.34 mmol) were stirred in DMF (8.0 mL) for 6 h at 130° C. TFA (1.0 mL, 13 mmol) was added and the mixture was stirred for 1 h at 130° C. Water was added to the mixture, filtered, washed with water and the solid was dried under reduced pressure at 60° C. to give 537 mg (94% yield) of the target compound, which was used without further purification.

LC-MS (Method 2): Rₜ=0.86 min; MS (ESIpos): m/z=425 [M+H]⁺

¹H-NMR (400 MHz, DMSO-d₆): δ [ppm]=7.41-7.48 (m, 2H), 7.59 (ddd, 1H), 7.74 (ddd, 1H), 7.91 (dd, 1H), 8.23 (dd, 1H), 8.42 (d, 1H), 12.45 (s, 1H).

### Intermediate 461

### 4-[5-oxo-7-(trifluoromethyl)-5,6-dihydro[1,2,4]triazolo[1,5-c]quinazolin-2-yl]benzonitrile

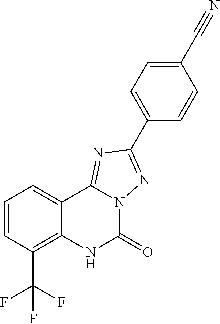 [SMILES: N#Cc1ccc(-c2nc3c4cccc(C(F)(F)F)c4[nH]c(=O)n3n2)cc1]

methyl [2-cyano-6-(trifluoromethyl)phenyl]carbamate (350 mg, 1.43 mmol) and 4-cyanobenzohydrazide (231 mg, 1.43 mmol) were stirred in DMF (15 ml) at 120° C. for 20 h. The reaction mixture was cooled to room temperature and then water was added to the mixture, filtered, washed with water and the solid was dried under reduced pressure at 50° C. to give 332 mg of a raw material, which was stirred in acetic acid (2 ml) at 90° C. for 20 h. The reaction mixture was cooled to room temperature, water was added, filtered, washed with water and the solid was dried under reduced pressure to give 281 mg (52% yield) of the target compound, which was used without further purification.

LC-MS (Method 1): Rₜ=1.14 min; MS (ESIpos): m/z=356 [M+H]⁺

¹H-NMR (400 MHz, DMSO-d₆): δ [ppm]=7.60 (t, 1H), 8.06 (d, 2H), 8.11 (br d, 1H), 8.39 (d, 2H), 8.57 (d, 1H), 11.72 (br s, 1H).

The following intermediates were prepared following the procedure described for intermediate 461:

| | Structure |
|---|---|
| | IUPAC-Name |
| | LC-MS (method): Retention time; Mass found |
| Intermediate | ¹H-NMR |
| Intermediate 462 | 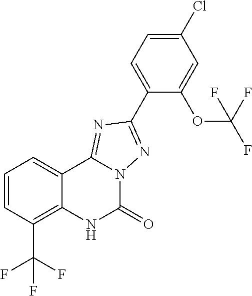 [SMILES: O=c1[nH]c2c(C(F)(F)F)cccc2c2nc(-c3ccc(Cl)cc3OC(F)(F)F)nn12] |
| | 2-[4-chloro-2-(trifluoromethoxy)phenyl]-7- |
| | (trifluoromethyl)[1,2,4]triazolo[1,5-c] |
| | quinazolin-5(6H)-one |
| | LC-MS (Method 1): Rt = 1.43 min; |
| | MS (ESIpos): m/z = 449 [M + H]⁺ |
| | 1H-NMR (400 MHz, DMSO-d₆): δ [ppm] = |
| | 7.60 (t, 1H), 7.74-7.80 (m, 2H), 8.10 (dd, 1H), |
| | 8.34 (d, 1H), 8.51 (dd, 1H), 11.69 (br s, 1H). |
| Intermediate 463 | 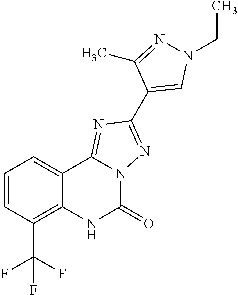 [SMILES: CCn1cc(-c2nc3c4cccc(C(F)(F)F)c4[nH]c(=O)n3n2)c(C)n1] |
| | 2-(1-ethyl-3-methyl-1H-pyrazol-4-yl)-7- |
| | (trifluoromethyl)[1,2,4]triazolo[1,5-c] |
| | quinazolin-5(6H)-one |
| | LC-MS (Method 1): Rt = 1.02 min; |
| | MS (ESIpos): m/z = 363 [M + H]⁺ |
| | ¹H-NMR (400 MHz, DMSO-d₆): δ [ppm] = |
| | 1.41 (t, 3H), 2.54 (s, 3H), 4.16 (q, 2H), 7.56 |
| | (t, 1H), 8.07 (dd, 1H), 8.36 (s, 1H), 8.48 |
| | (dd, 1H), 11.53 (br s, 1H). |
| Intermediate 464 | 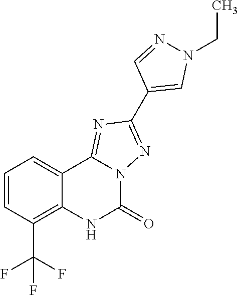 [SMILES: CCn1cc(-c2nc3c4cccc(C(F)(F)F)c4[nH]c(=O)n3n2)cn1] |
| | 2-(1-ethyl-1H-pyrazol-4-yl)-7- |
| | (trifluoromethyl)[1,2,4]triazolo[1,5-c] |
| | quinazolin-5(6H)-one |
| | LC-MS (Method 1): Rt = 0.95 min; |
| | MS (ESIpos): m/z = 349 [M + H]⁺ |
| | ¹H-NMR (400 MHz, DMSO-d₆): δ [ppm] = |
| | 1.44 (t, 3H), 4.25 (q, 2H), 7.56 (t, 1H), 8.04 |
| | (d, 1H), 8.07 (dd, 1H), 8.47-8.51 (m, 2H), |
| | 11.53 (br s, 1H). |
| Intermediate 465 | 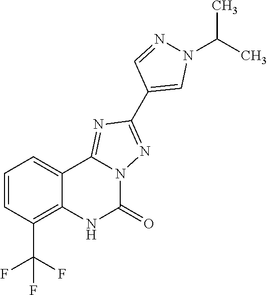 [SMILES: CC(C)n1cc(-c2nc3c4cccc(C(F)(F)F)c4[nH]c(=O)n3n2)cn1] |
| | 2-[1-(propan-2-yl)-1H-pyrazol-4-yl]-7- |
| | (trifluoromethyl)[1,2,4]triazolo[1,5-c] |
| | quinazolin-5(6H)-one |
| | LC-MS (Method 1): Rt = 1.03 min; |
| | MS (ESIpos): m/z = 363 [M + H]⁺ |
| | ¹H-NMR (400 MHz, DMSO-d₆) δ [ppm] = |
| | 1.49 (d, 6H), 4.63 (spt, 1H), 7.57 (t, 1H), 8.05 |
| | (d, 1H), 8.08 (dd, 1H), 8.48-8.52 (m, 2H), |
| | 11.53 (br s, 1H). |
| Intermediate 466 | 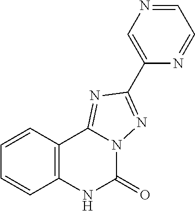 [SMILES: O=c1[nH]c2ccccc2c2nc(-c3cnccn3)nn12] |
| | 2-(pyrazin-2-yl)[1,2,4]triazolo[1,5-c] |
| | quinazolin-5(6H)-one |
| | LC-MS (Method 2): Rₜ = 0.49 min; |
| | MS (ESIpos): m/z = 265 [M + H]⁺ |
| | ¹H-NMR (400 MHz, DMSO-d₆): δ [ppm] = |
| | 7.09-7.50 (m, 2H), 7.75 (ddd, 1H), 8.26 |
| | (dd, 1H), 8.81-8.89 (m, 2H), 9.46 (d, 1H), |
| | 12.45 (br s, 1H). |
| Intermediate 467 | 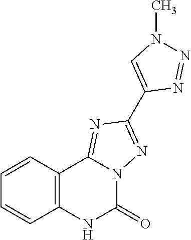 [SMILES: Cn1cc(-c2nc3c4ccccc4[nH]c(=O)n3n2)nn1] |
| | 2-(1-methyl-1H-1,2,3-triazol-4-yl)[1,2,4] |
| | triazolo[1,5-c]quinazolin-5(6H)-one |
| | LC-MS (Method 2): Rₜ = 0.48 min; |
| | MS (ESIpos): m/z = 268 [M + H]⁺ |
| | ¹H-NMR (400 MHz, DMSO-d₆): δ [ppm] = |
| | 4.16 (s, 3H), 7.40-7.44 (m, 1H), 7.46 (d, 1H), |
| | 7.72 (ddd, 1H), 8.20 (dd, 1H), 8.79 (s, 1H), |
| | 12.36 (br s, 1H). |
| Intermediate 468 | 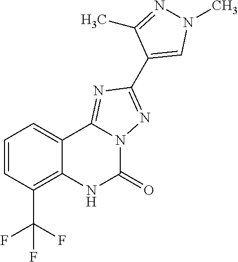 [SMILES: Cc1nn(C)cc1-c1nc2c3cccc(C(F)(F)F)c3[nH]c(=O)n2n1] |
| | 2-(1,3-dimethyl-1H-pyrazol-4-yl)-7- |
| | (trifluoromethyl)[1,2,4]triazolo[1,5-c] |
| | quinazolin-5(6H)-one |
| | LC-MS (Method 1): Rt = 0.95 min; |
| | MS (ESIpos): m/z = 349 [M + H]⁺ |
| | ¹H-NMR (400 MHz, DMSO-d₆): δ [ppm] = |
| | 2.53 (s, 3H), 3.86 (s, 3H), 7.56 (t,1H), 8.07 |
| | (d, 1H), 8.32 (s, 1H), 8.48 (dd, 1H), |
| | 11.53 (br s, 1H). |

### Intermediate 469

### 2-[4-chloro-2-(difluoromethoxy)phenyl]-7-(trifluoromethyl)[1,2,4]triazolo[1,5-c]quinazolin-5(6H)-one

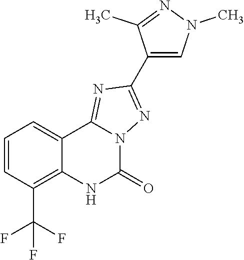 [SMILES: Cc1nn(C)cc1-c1nc2c3cccc(C(F)(F)F)c3[nH]c(=O)n2n1]

Di-tert-butyl [2-cyano-6-(trifluoromethyl)phenyl]-2-imidodicarbonate (200 mg, 518 μmol) and 4-chloro-2-(difluoromethoxy)benzohydrazide (147 mg, 621 μmol) were stirred in DMF (1.7 ml) at 120° C. for 20 h. Then acetic acid (2 ml) was added at 100° C. and the reaction mixture was stirred for 24 h at this temperature. The reaction mixture was cooled to room temperature and added to water. After stirring for 10 minutes filtered, washed with water and the solid was dried under reduced pressure at 60° C. to give 182 mg (74% yield) of the target compound, which was used without further purification.

LC-MS (Method 1): Rₜ=1.33 min; MS (ESIpos): m/z=431 [M+H]⁺

¹H-NMR (400 MHz, DMSO-d₆): δ [ppm]=7.39 (t, 1H), 7.54-7.62 (m, 3H), 8.10 (d, 1H), 8.21 (d, 1H), 8.50-8.56 (m, 1H), 11.68 (br s, 11H).

The following intermediates were prepared similarly to intermediate 469:

| | Structure |
|---|---|
| | IUPAC-Name |
| | LC-MS (method): Retention time; |
| | Mass found |
| Intermediate | ¹H-NMR |
| Intermediate 470 | 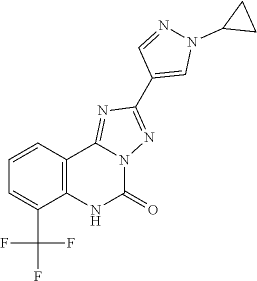 [SMILES: O=c1[nH]c2c(C(F)(F)F)cccc2c2nc(-c3cnn(C4CC4)c3)nn12] |
| | 2-(1-cyclopropyl-1H-pyrazol-4-yl)-7- |
| | (trifluoromethyl)[1,2,4]triazolo[1,5-c] |
| | quinazolin-5(6H)-one |
| | LC-MS (Method 1): Rt = 0.98 min; |
| | MS (ESIpos): m/z = 359 [M − H]⁺ |
| | ¹H-NMR (400 MHz, DMSO-d₆): δ [ppm] = |
| | 0.99-1.05 (m, 2H), 1.13-1.19 (m, 2H), 3.88 |
| | (tt, 1H), 7.56 (t, 1H), 8.03 (d, 1H), 8.07 |
| | (d, 1H), 8.47-8.52 (m, 2H), 11.54 (br s, 1H). |
| Intermediate 471 | 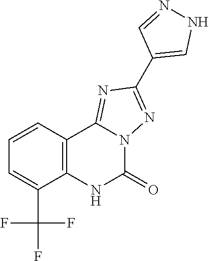 [SMILES: O=c1[nH]c2c(C(F)(F)F)cccc2c2nc(-c3cn[nH]c3)nn12] |
| | 2-(1H-pyrazol-4-yl)-7-(trifluoromethyl) |
| | [1,2,4]triazolo[1,5-c]quinazolin-5(6H)-one |
| | LC-MS (Method 1): Rt = 0.82 min; |
| | MS (ESIpos): m/z = 319 [M − H]⁺ |
| | ¹H-NMR (400 MHz, DMSO-d₆): δ [ppm] = |
| | 7.56 (t, 1H), 8.08 (dd, 1H), 8.10 (br s, 1H), |
| | 8.45 (br s, 1H), 8.51 (dd, 1H), 11.53 |
| | (br s, 1H), 13.35 (br s, 1H). |

### Intermediate 472

### 5-chloro-2-(1-methyl-1H-pyrazol-3-yl)[1,2,4]triazolo[1,5-c]quinazoline

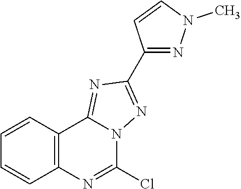 [SMILES: Cn1ccc(-c2nc3c4ccccc4nc(Cl)n3n2)n1]

2-(1-Methyl-1H-pyrazol-3-yl)[1,2,4]triazolo[1,5-c]quinazolin-5(6H)-one (630 mg, 2.37 mmol) was suspended in phosphorus oxychloride (6.0 mL), DIPEA (4.1 mL, 24 mmol) was added carefully and the mixture was stirred for 3 h at 110° C. The mixture was poured into ice, stirred for 1 h, filtered, washed with water and dried at 60° C. under reduced pressure to give 485 mg (100% purity, 68% yield) of the target compound, which was used without further purification.

LC-MS (Method 2): Rₜ=1.00 min; MS (ESIpos): m/z=285 [M+H]⁺

¹H-NMR (400 MHz, DMSO-d6): δ [ppm]=3.99 (s, 3H), 6.94 (d, 1H), 7.86 (ddd, 1H), 7.91 (d, 1H), 7.94-8.00 (m, 1H), 8.01-8.06 (m, 1H), 8.50 (dd, 1H).

The following intermediates were prepared similarly to intermediate 472:

| | Structure |
|---|---|
| | IUPAC-Name |
| | LC-MS (method): Retention time; Mass found |
| Intermediate | ¹H-NMR |
| Intermediate 473 | 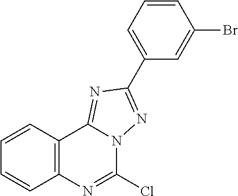 [SMILES: Clc1nc2ccccc2c2nc(-c3cccc(Br)c3)nn12] |
| | 2-(3-bromophenyl)-5-chloro[1,2,4]triazolo |
| | [1,5-c]quinazoline |
| | LC-MS (Method 2): Rt = 1.56 min; |
| | MS (ESIpos): m/z = 359 [M + H]⁺ |
| | ¹H-NMR (400 MHz, DMSO-d₆): δ [ppm] = |
| | 7.59 (t, 1H), 7.80-7.84 (m, 1H), 7.86-7.91 |
| | (m, 1H), 8.00 (ddd, 1H), 8.04-8.08 (m, 1H), |
| | 8.30 (dt, 1H), 8.41 (t, 1H), 8.55 (dd, 1H). |
| Intermediate 474 | 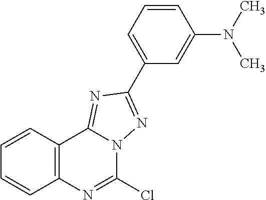 [SMILES: CN(C)c1cccc(-c2nc3c4ccccc4nc(Cl)n3n2)c1] |
| | 3-(5-chloro[1,2,4]triazolo[1,5-c]quinazolin- |
| | 2-yl)-N,N-dimethylaniline |
| | LC-MS (Method 2): Rt = 1.42 min; |
| | MS (ESIpos): m/z = 324 [M + H]⁺ |
| | ¹H-NMR (400 MHz, DMSO-d₆): δ [ppm] = |
| | 3.02 (s, 6H), 6.92-6.96 (m, 1H), 7.40 (t, 1H), |
| | 7.58-7.63 (m, 2H), 7.86 (ddd, 1H), 7.95-8.00 |
| | (m, 1H), 8.03-8.06 (m, 1H), 8.54 (dd, 1H). |
| Intermediate 475 | 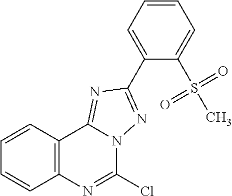 [SMILES: CS(=O)(=O)c1ccccc1-c1nc2c3ccccc3nc(Cl)n2n1] |
| | 5-chloro-2-[2-(methanesulfonyl)phenyl] |
| | [1,2,4]triazolo[1,5-c]quinazoline |
| | LC-MS (Method 2): Rt = 1.10 min; |
| | MS (ESIpos): m/z = 359 [M + H]⁺ |
| | ¹H-NMR (400 MHz, DMSO-d₆): δ [ppm] = |
| | 3.74 (s, 3H), 7.89-7.94 (m, 2H), 7.95-7.97 |
| | (m, 2H), 8.01-8.06 (m, 1H), 8.09-8.13 (m, |
| | 1H), 8.20-8.23 (m, 1H), 8.53 (dd, 1H). |
| Intermediate 476 | 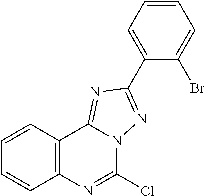 [SMILES: Clc1nc2ccccc2c2nc(-c3ccccc3Br)nn12] |
| | 2-(2-bromophenyl)-5-chloro[1,2,4] |
| | triazolo[1,5-c]quinazoline |
| | LC-MS (Method 1): Rt = 1.39 min; |
| | MS (ESIpos): m/z = 359 [M + H]⁺ |
| Intermediate 477 | 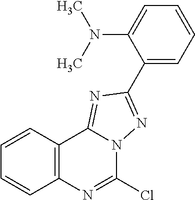 [SMILES: CN(C)c1ccccc1-c1nc2c3ccccc3nc(Cl)n2n1] |
| | 2-(5-chloro[1,2,4]triazolo[1,5-c]quinazolin- |
| | 2-yl)-N,N-dimethylaniline |
| | LC-MS (Method 2): Rt = 1.33 min; |
| | MS (ESIpos): m/z = 324 [M + H]⁺ |
| | ¹H-NMR (400 MHz, DMSO-d₆): δ [ppm] = |
| | 2.68-2.74 (m, 6H), 7.00-7.07 (m, 1H), 7.12- |
| | 7.19 (m, 1H), 7.40-7.46 (m, 1H), 7.79 (br d, |
| | 1H), 7.83-7.89 (m, 1H), 7.99 (dd, 1H), 8.05 |
| | (d, 1H), 8.51 (d, 1H). |
| Intermediate 478 | 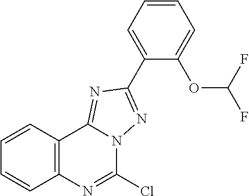 [SMILES: FC(F)Oc1ccccc1-c1nc2c3ccccc3nc(Cl)n2n1] |
| | 5-chloro-2-[2-(difluoromethoxy)phenyl] |
| | [1,2,4]triazolo[1,5-c]quinazoline |
| | LC-MS (Method 2): Rt = 1.35 min; |
| | MS (ESIpos): m/z = 347 [M + H]⁺ |
| | ¹H-NMR (400 MHz, DMSO-d₆): δ [ppm] = |
| | 7.30 (t, 1H), 7.42-7.46 (m, 1H), 7.52 (td, 1H), |
| | 7.65-7.70 (m, 1H), 7.88 (ddd, 1H), 8.00 (ddd, |
| | 1H), 8.05-8.08 (m, 1H), 8.26 (dd, 1H), 8.51 |
| | (dd, 1H). |
| Intermediate 479 | 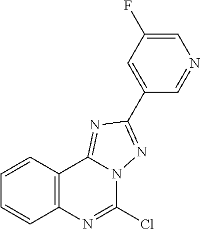 [SMILES: Fc1cncc(-c2nc3c4ccccc4nc(Cl)n3n2)c1] |
| | 5-chloro-2-(5-fluoropyridin-3-yl)[1,2,4] |
| | triazolo[1,5-c]quinazoline |
| | LC-MS (Method 2): Rt = 1.21 min; |
| | MS (ESIpos): m/z = 300 [M + H]⁺ |
| | ¹H-NMR (400 MHz, DMSO-d₆): δ [ppm] = |
| | 7.90 (ddd, 1H), 8.01 (ddd, 1H), 8.06-8.09 |
| | (m, 1H), 8.44 (ddd, 1H), 8.55 (ddd, 1H), 8.83 |
| | (d, 1H), 9.31 (t, 1H). |
| Intermediate 480 | 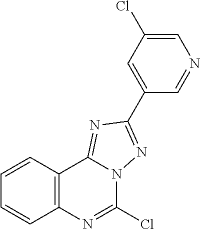 [SMILES: Clc1cncc(-c2nc3c4ccccc4nc(Cl)n3n2)c1] |
| | 5-chloro-2-(5-chloropyridin-3-yl)[1,2,4] |
| | triazolo[1,5-c]quinazoline |
| | LC-MS (Method 2): Rt = 1.34 min; |
| | MS (ESIpos): m/z = 316 [M + H]⁺ |
| | ¹H-NMR (400 MHz, DMSO-d₆): δ [ppm] = |
| | 7.90 (ddd, 1H), 7.99-8.04 (m, 1H), 8.06-8.09 |
| | (m, 1H), 8.55 (ddd, 1H), 8.63 (ddd, 1H), 8.87 |
| | (d, 1H), 9.38 (d, 1H). |
| Intermediate 481 | 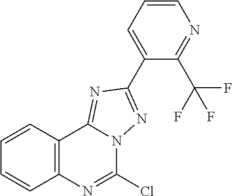 [SMILES: FC(F)(F)c1ncccc1-c1nc2c3ccccc3nc(Cl)n2n1] |
| | 5-chloro-2-[2-(trifluoromethyl)pyridin-3-yl] |
| | [1,2,4]triazolo[1,5-c]quinazoline |
| | LC-MS (Method 2): Rt = 1.23 min; |
| | MS (ESIpos): m/z = 350 [M + H]⁺ |
| | ¹H-NMR (400 MHz, DMSO-d₆): δ [ppm] = |
| | 7.90 (ddd, 1H), 7.97 (dd, 1H), 8.00-8.05 |
| | (m, 1H), 8.08-8.11 (m, 1H), 8.48-8.53 |
| | (m, 2H), 8.98 (dd, 1H). |
| Intermediate 482 | 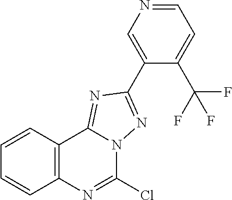 [SMILES: FC(F)(F)c1ccncc1-c1nc2c3ccccc3nc(Cl)n2n1] |
| | 5-chloro-2-[4-(trifluoromethyl)pyridin-3-yl] |
| | [1,2,4]triazolo[1,5-c]quinazoline |
| | LC-MS (Method 2): Rt = 1.24 min; |
| | MS (ESIpos): m/z = 350 [M + H]⁺ |
| | ¹H-NMR (400 MHz, DMSO-d₆): δ [ppm] = |
| | 7.90 (ddd, 1H), 8.00-8.07 (m, 2H), 8.09 (d, |
| | 1H), 8.52 (dd, 1H), 9.09 (d, 1H), 9.30 (s, 1H). |
| Intermediate 483 | 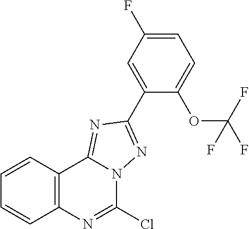 [SMILES: Fc1ccc(OC(F)(F)F)c(-c2nc3c4ccccc4nc(Cl)n3n2)c1] |
| | 5-chloro-2-[5-fluoro-2-(trifluoromethoxy) |
| | phenyl][1,2,4]triazolo[1,5-c]quinazoline |
| | LC-MS (Method 2): Rt = 1.53 min; |
| | MS (ESIpos): m/z = 383 [M + H]⁺ |
| | ¹H-NMR (400 MHz, DMSO-d₆): δ [ppm] = |
| | 7.61 (ddd, 1H), 7.69-7.74 (m, 1H), 7.89 |
| | (ddd, 1H), 8.01 (ddd, 1H), 8.05-8.09 (m, 1H), |
| | 8.14 (dd, 1H), 8.51 (dd, 1H). |
| Intermediate 484 | 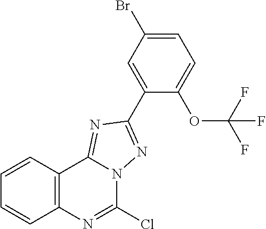 [SMILES: FC(F)(F)Oc1ccc(Br)cc1-c1nc2c3ccccc3nc(Cl)n2n1] |
| | 2-[5-bromo-2-(trifluoromethoxy)phenyl]-5- |
| | chloro[1,2,4]triazolo[1,5-c]quinazoline |
| | LC-MS (Method 2): Rt = 1.64 min; |
| | MS (ESIpos): m/z = 443 [M + H]⁺ |
| | ¹H-NMR (400 MHz, DMSO-d₆): δ [ppm] = |
| | 7.61-7.65 (m, 1H), 7.89 (ddd, 1H), 7.95 (dd, |
| | 1H), 7.98-8.03 (m, 1H), 8.06-8.09 (m, 1H), |
| | 8.52 (d, 1H), 8.53-8.55 (m, 1H). |
| Intermediate 485 | 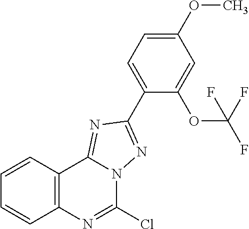 [SMILES: COc1ccc(-c2nc3c4ccccc4nc(Cl)n3n2)c(OC(F)(F)F)c1] |
| | 5-chloro-2-[4-methoxy-2-(trifluoromethoxy) |
| | phenyl][1,2,4]triazolo[1,5-c]quinazoline |
| | LC-MS (Method 2): Rt = 1.49 min; |
| | MS (ESIpos): m/z = 395 [M + H]⁺ |
| | ¹H-NMR (400 MHz, DMSO-d₆): δ [ppm] = |
| | 3.92 (s, 3H), 7.13 (dd, 1H), 7.26 (dd, 1H), |
| | 7.87 (ddd, 1H), 7.98 (ddd, 1H), 8.04 (s, 1H), |
| | 8.35 (d, 1H), 8.49 (dd, 1H). |
| Intermediate 486 | 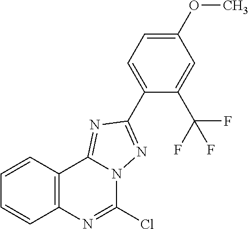 [SMILES: COc1ccc(-c2nc3c4ccccc4nc(Cl)n3n2)c(C(F)(F)F)c1] |
| | 5-chloro-2-[4-methoxy-2-(trifluoromethyl) |
| | phenyl][1,2,4]triazolo[1,5-c]quinazoline |
| | LC-MS (Method 2): Rt = 1.42 min; |
| | MS (ESIpos): m/z = 379 [M + H]⁺ |
| | ¹H-NMR (400 MHz, DMSO-d₆) δ [ppm]: |
| | 1.196 (2.47), 1.211 (2.52), 1.241 (1.22), |
| | 1.257 (1.37), 2.326 (1.60), 2.669 (1.74), |
| | 3.398 (7.40), 3.882 (13.31), 3.917 (2.67), |
| | 3.948 (16.00), 3.983 (1.12), 7.284 (1.89), |
| | 7.302 (5.06), 7.383 (1.07), 7.442 (2.79), |
| | 7.462 (6.40), 7.859 (2.67), 7.878 (3.46), |
| | 7.897 (1.64), 7.980 (1.27), 7.999 (2.17), |
| | 8.019 (3.12), 8.042 (2.34), 8.059 (3.02), |
| | 8.080 (1.77), 8.477 (2.09), 8.496 (1.92). |
| Intermediate 487 | 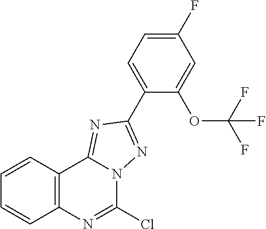 [SMILES: Fc1ccc(-c2nc3c4ccccc4nc(Cl)n3n2)c(OC(F)(F)F)c1] |
| | 5-chloro-2-[4-fluoro-2-(trifluoromethoxy) |
| | phenyl][1,2,4]triazolo[1,5-c]quinazoline |
| | LC-MS (Method 1): Rt = 1.50 min; |
| | MS (ESIpos): m/z = 383 [M + H]⁺ |
| Intermediate 488 | 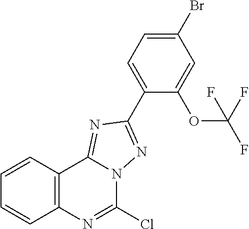 [SMILES: FC(F)(F)Oc1cc(Br)ccc1-c1nc2c3ccccc3nc(Cl)n2n1] |
| | 2-[4-bromo-2-(trifluoromethoxy)phenyl]- |
| | 5-chloro[1,2,4]triazolo[1,5-c]quinazoline |
| | LC-MS (Method 2): Rt =1 .63 min; |
| | MS (ESIpos): m/z = 443 [M + H]⁺ |
| | ¹H-NMR (400 MHz, DMSO-d₆): δ [ppm] = |
| | 7.86-7.94 (m, 3H), 7.98-8.03 (m, 1H), 8.05- |
| | 8.09 (m, 1H), 8.36 (d, 1H), 8.50 (dd, 1H). |
| Intermediate 489 | 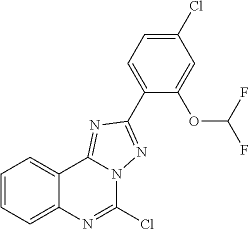 [SMILES: FC(F)Oc1cc(Cl)ccc1-c1nc2c3ccccc3nc(Cl)n2n1] |
| | 5-chloro-2-[4-chloro-2-(difluoromethoxy) |
| | phenyl][1,2,4]triazolo[1,5-c]quinazoline |
| | LC-MS (Method 1): Rt = 1.45 min; |
| | MS (ESIpos): m/z = 381 [M + H]⁺ |
| | ¹H-NMR (400 MHz, DMSO-d₆): δ [ppm] = |
| | 7.39 (t, 1H), 7.56-7.59 (m, 1H), 7.62 (dd, 1H), |
| | 7.88 (ddd, 1H), 8.00 (ddd, 1H), 8.04-8.08 |
| | (m, 1H), 8.29 (d, 1H), 8.50 (ddd, 1H). |
| Intermediate 491 | 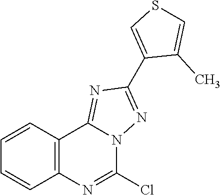 [SMILES: Cc1cscc1-c1nc2c3ccccc3nc(Cl)n2n1] |
| | 5-chloro-2-(4-methylthiophen-3-yl)[1,2,4] |
| | triazolo[1,5-c]quinazoline |
| | LC-MS (Method 1): Rt = 1.46 min; |
| | MS (ESIpos): m/z = 301 [M + H]⁺ |
| | ¹H-NMR (400 MHz, DMSO-d₆): δ [ppm] = |
| | 2.65 (s, 3H), 7.42 (dd, 1H), 7.83-7.88 (m, |
| | 1H), 7.97 (ddd, 1H), 8.02-8.06 (m, 1H), 8.38 |
| | (d, 1H), 8.49 (dd, 1H). |
| Intermediate 492 | 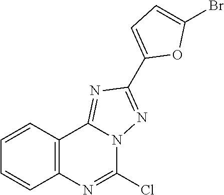 [SMILES: Clc1nc2ccccc2c2nc(-c3ccc(Br)o3)nn12] |
| | 2-(5-bromofuran-2-yl)-5-chloro[1,2,4] |
| | triazolo[1,5-c]quinazoline |
| | LC-MS (Method 2): Rₜ = 1.33 min; |
| | MS (ESIpos): m/z = 349 [M + H]⁺ |
| | ¹H-NMR (400 MHz, DMSO-d₆): δ [ppm] = |
| | 6.92 (d, 1H), 7.42 (d, 1H), 7.86 (ddd, 1H), |
| | 7.96-8.02 (m, 1H), 8.03-8.07 (m, 1H), |
| | 8.49 (dd, 1H). |
| Intermediate 493 | 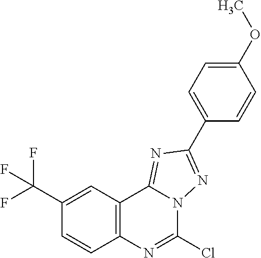 [SMILES: COc1ccc(-c2nc3c4cc(C(F)(F)F)ccc4nc(Cl)n3n2)cc1] |
| | 5-chloro-2-(4-methoxyphenyl)-9- |
| | (trifluoromethyl)[1,2,4]triazolo[1,5-c] |
| | quinazoline |
| | LC-MS (Method 2): Rₜ = 1.51 min; |
| | MS (ESIpos): m/z = 379 [M + H]⁺ |
| | ¹H-NMR (400 MHz, DMSO-d₆): δ [ppm] = |
| | 3.87 (s, 3H), 7.15-7.19 (m, 2H), 8.24-8.28 |
| | (m, 4H), 8.73-8.78 (m, 1H). |
| Intermediate 494 | 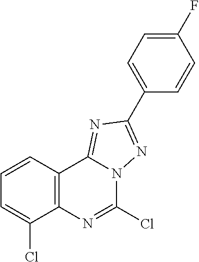 [SMILES: Fc1ccc(-c2nc3c4cccc(Cl)c4nc(Cl)n3n2)cc1] |
| | 5,7-dichloro-2-(4-fluorophenyl)[1,2,4] |
| | triazolo[1,5-c]quinazoline |
| | LC-MS (Method 2): Rₜ = 1.46 min; |
| | MS (ESIpos): m/z = 333 [M + H]⁺ |
| | ¹H-NMR (400 MHz, DMSO-d₆): δ [ppm] = |
| | 7.42-7.49 (m, 2H), 7.84 (t, 1H), 8.15 (dd, |
| | 1H), 8.32-8.38 (m, 2H), 8.49 (dd, 1H). |
| Intermediate 495 | 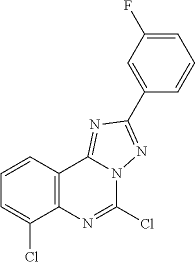 [SMILES: Fc1cccc(-c2nc3c4cccc(Cl)c4nc(Cl)n3n2)c1] |
| | 5,7-dichloro-2-(3-fluorophenyl)[1,2,4] |
| | triazolo[1,5-c]quinazoline |
| | LC-MS (Method 2): Rₜ = 1.48 min; |
| | MS (ESIpos): m/z = 333 [M + H]⁺ |
| | ¹H-NMR (400 MHz, DMSO-d₆): δ [ppm] = |
| | 7.44-7.50 (m, 1H), 7.68 (td, 1H), 7.85 |
| | (t, 1H), 7.97-8.03 (m, 1H), 8.13-8.18 |
| | (m, 2H), 8.50 (dd, 1H). |
| Intermediate 496 | 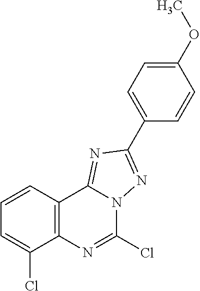 [SMILES: COc1ccc(-c2nc3c4cccc(Cl)c4nc(Cl)n3n2)cc1] |
| | 5,7-dichloro-2-(4-methoxyphenyl)[1,2,4] |
| | triazolo[1,5-c]quinazoline |
| | LC-MS (Method 2): Rₜ = 1.43 min; |
| | MS (ESIpos): m/z = 345 [M + H]⁺ |
| | ¹H-NMR (400 MHz, DMSO-d₆): δ [ppm] = |
| | 3.87 (s, 3H), 7.12-7.20 (m, 2H), 7.82 (t, 1H), |
| | 8.13 (dd, 1H), 8.20-8.28 (m, 2H), 8.47 |
| | (dd, 1H). |
| Intermediate 497 | 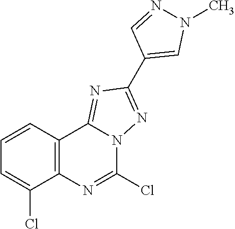 [SMILES: Cn1cc(-c2nc3c4cccc(Cl)c4nc(Cl)n3n2)cn1] |
| | 5,7-dichloro-2-(1-methyl-1H-pyrazol-4-yl) |
| | [1,2,4]triazolo[1,5-c]quinazoline |
| | LC-MS (Method 2): Rₜ = 1.08 min; |
| | MS (ESIpos): m/z = 319 [M + H]⁺ |
| | ¹H-NMR (400 MHz, DMSO-d₆): δ [ppm] = |
| | 3.96 (s, 3H), 7.81 (t, 1H), 8.09- |
| | 8.14 (m, 2H), 8.42 (dd, 1H), 8.55 (s, 1H). |
| Intermediate 498 | 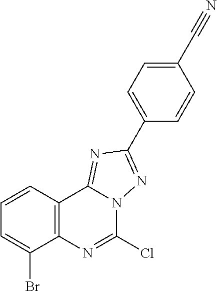 [SMILES: N#Cc1ccc(-c2nc3c4cccc(Br)c4nc(Cl)n3n2)cc1] |
| | 4-(7-bromo-5-chloro[1,2,4]triazolo[1,5-c] |
| | quinazolin-2-yl)benzonitrile |
| | LC-MS (Method 2): Rt = 1.41 min; |
| | MS (ESIpos): m/z = 386 [M + H]⁺ |
| | ¹H-NMR (400 MHz, DMSO-d₆): δ [ppm] = |
| | 7.78 (t, 1H), 8.07-8.11 (m, 2H), 8.33 |
| | (dd, 1H), 8.44-8.48 (m, 2H), 8.54 (dd, 1H). |
| Intermediate 499 | 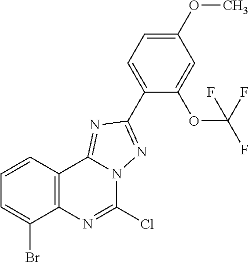 [SMILES: COc1ccc(-c2nc3c4cccc(Br)c4nc(Cl)n3n2)c(OC(F)(F)F)c1] |
| | 7-bromo-5-chloro-2-[4-methoxy-2- |
| | (trifluoromethoxy)phenyl][1,2,4]triazolo |
| | [1,5-c]quinazoline |
| | LC-MS (Method 2): Rt = 1.58 min; |
| | MS (ESIpos): m/z = 473 [M + H]⁺ |
| | ¹H-NMR (400 MHz, DMSO-d₆): δ [ppm] = |
| | 3.92 (s, 3H), 7.14 (br s, 1H), 7.26 (dd, 1H), |
| | 7.76 (br t, 1H), 8.31 (br d, 1H), 8.36 (d, 1H), |
| | 8.49 (br d, 1H). |
| Intermediate 500 | 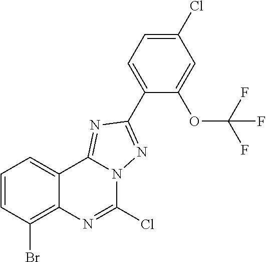 [SMILES: FC(F)(F)Oc1cc(Cl)ccc1-c1nc2c3cccc(Br)c3nc(Cl)n2n1] |
| | 7-bromo-5-chloro-2-[4-chloro-2- |
| | (trifluoromethoxy)phenyl][1,2,4]triazolo |
| | [1,5-c]quinazoline |
| | LC-MS (Method 2): Rt = 1.62 min; |
| | MS (ESIpos): m/z = 477 [M + H]⁺ |
| | ¹H-NMR (400 MHz, DMSO-d₆): δ [ppm] = |
| | 7.76-7.81 (m, 2H), 7.82 (d, 1H), 8.33 (dd, |
| | 1H), 8.45 (d, 1H), 8.50 (dd, 1H). |
| Intermediate 501 | 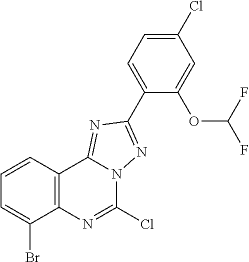 [SMILES: FC(F)Oc1cc(Cl)ccc1-c1nc2c3cccc(Br)c3nc(Cl)n2n1] |
| | 7-bromo-5-chloro-2-[4-chloro-2- |
| | (difluoromethoxy)phenyl][1,2,4]triazolo |
| | [1,5-c]quinazoline |
| | ¹H-NMR (400 MHz, DMSO-d₆): δ [ppm] = |
| | 7.41 (t, 1H), 7.57-7.60 (m, 1H), 7.62 (dd, 1H), |
| | 7.77 (t, 1H), 8.30 (d, 1H), 8.31 (dd, 1H), 8.51 |
| | (dd, 1H). |
| Intermediate 502 | 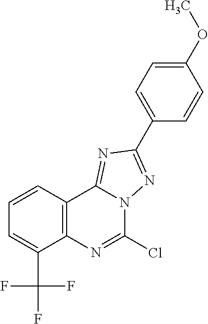 [SMILES: COc1ccc(-c2nc3c4cccc(C(F)(F)F)c4nc(Cl)n3n2)cc1] |
| | 5-chloro-2-(4-methoxyphenyl)-7- |
| | (trifluoromethyl)[1,2,4]triazolo[1,5-c] |
| | quinazoline |
| | LC-MS (Method 2): Rₜ = 1.46 min; |
| | MS (ESIpos): m/z = 379 [M + H]⁺ |
| Intermediate 503 | 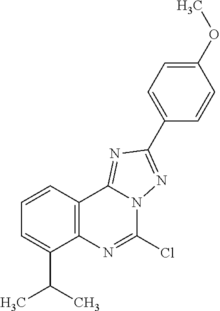 [SMILES: COc1ccc(-c2nc3c4cccc(C(C)C)c4nc(Cl)n3n2)cc1] |
| | 5-chloro-2-(4-methoxyphenyl)-7-(propan-2- |
| | yl)[1,2,4]triazolo[1,5-c]quinazoline |
| | LC-MS (Method 2): Rₜ = 1.63 min; |
| | MS (ESIpos): m/z = 353 [M + H]⁺ |
| | ¹H-NMR (400 MHz, DMSO-d₆): δ [ppm] = |
| | 1.34 (d, 6H), 3.86 (s, 3H), 4.00-4.12 (m, 1H), |
| | 7.13-7.18 (m, 2H), 7.81 (t, 1H), 7.89 (dd, 1H), |
| | 8.21-8.26 (m, 2H), 8.35 (dd, 1H). |
| Intermediate 504 | 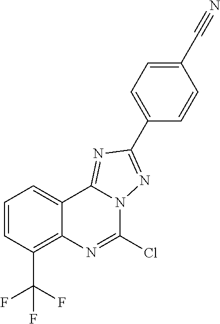 [SMILES: N#Cc1ccc(-c2nc3c4cccc(C(F)(F)F)c4nc(Cl)n3n2)cc1] |
| | 4-[5-chloro-7-(trifluoromethyl)[1,2,4]triazolo |
| | [1,5-c]quinazolin-2-yl]benzonitrile |
| | LC-MS (Method 1): Rt = 1.43 min; |
| | MS (ESIpos): m/z = 374 [M + H]⁺ |
| | ¹H-NMR (400 MHz, DMSO-d₆): δ [ppm] = |
| | 1.23 (br s, 1H), 2.52-2.56 (m, 1H), 3.47 (br s, |
| | 4H), 8.02 (t, 1H), 8.07-8.11 (m, 2H), 8.39 (dd, |
| | 1H), 8.44-8.49 (m, 2H), 8.82 (dd, 1H). |
| Intermediate 505 | 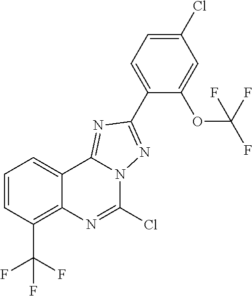 [SMILES: FC(F)(F)Oc1cc(Cl)ccc1-c1nc2c3cccc(C(F)(F)F)c3nc(Cl)n2n1] |
| | 5-chloro-2-[4-chloro-2-(trifluoromethoxy) |
| | phenyl]-7-(trifluoromethyl)[1,2,4] |
| | triazolo[1,5-c]quinazoline |
| | LC-MS (Method 1): Rt = 1.65 min; |
| | MS (ESIpos): m/z = 467 [M + H]⁺ |
| | ¹H-NMR (400 MHz, DMSO-d₆): δ [ppm] = |
| | 7.77-7.84 (m, 2H), 8.02 (t, 1H), 8.39 (dd, |
| | 1H), 8.45 (d, 1H), 8.78 (dd, 1H). |
| Intermediate 506 | 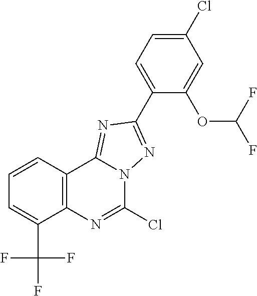 [SMILES: FC(F)Oc1cc(Cl)ccc1-c1nc2c3cccc(C(F)(F)F)c3nc(Cl)n2n1] |
| | 5-chloro-2-[4-chloro-2-(difluoromethoxy) |
| | phenyl]-7-(trifluoromethyl)[1,2,4]triazolo |
| | [1,5-c]quinazoline |
| | LC-MS (Method 1): Rt = 1.56 min; |
| | MS (ESIpos): m/z = 449 [M + H]⁺ |
| | ¹H-NMR (400 MHz, DMSO-d₆): δ [ppm] = |
| | 7.41 (t, 1H), 7.58-7.65 (m, 2H), 8.02 (t, 1H), |
| | 8.31 (d, 1H), 8.39 (br d, 1H), 8.80 (dd, 1H). |
| Intermediate 507 | 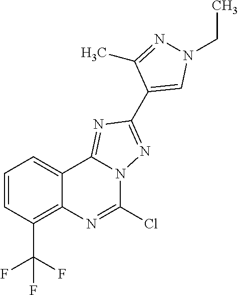 [SMILES: CCn1cc(-c2nc3c4cccc(C(F)(F)F)c4nc(Cl)n3n2)c(C)n1] |
| | 5-chloro-2-(1-ethyl-3-methyl-1H-pyrazol-4- |
| | yl)-7-(trifluoromethyl)[1,2,4]triazolo[1,5-c] |
| | quinazoline |
| | LC-MS (Method 1): Rt = 1.34 min; |
| | MS (ESIpos): m/z = 381 [M + H]⁺ |
| | ¹H-NMR (400 MHz, DMSO-d₆): δ [ppm] = |
| | 1.42 (t, 3H), 2.59 (s, 3H), 4.17 (q, 2H), 7.97 |
| | (t, 1H), 8.34 (dd, 1H), 8.47 (s, 1H), 8.72 |
| | (dd, 1H). |
| Intermediate 508 | 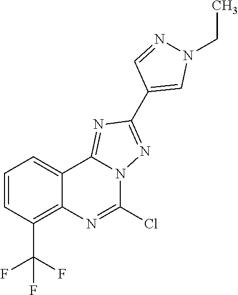 [SMILES: CCn1cc(-c2nc3c4cccc(C(F)(F)F)c4nc(Cl)n3n2)cn1] |
| | 5-chloro-2-(1-ethyl-1H-pyrazol-4-yl)-7- |
| | (trifluoromethyl)[1,2,4]triazolo[1,5-c] |
| | quinazoline |
| | LC-MS (Method 1): Rt = 1.23 min; |
| | MS (ESIpos): m/z = 367 [M + H]⁺ |
| | ¹H-NMR (400 MHz, DMSO-d₆): δ [ppm] = |
| | 1.45 (t, 3H), 4.26 (q, 2H), 7.97 (t, 1H), 8.12 |
| | (d, 1H), 8.34 (dd, 1H), 8.59 (d, 1H), 8.74 |
| | (dd, 1H). |
| Intermediate 509 | 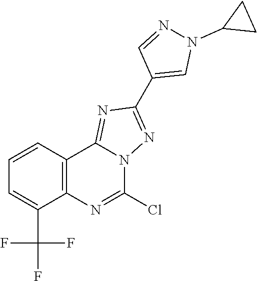 [SMILES: FC(F)(F)c1cccc2c1nc(Cl)n1nc(-c3cnn(C4CC4)c3)nc21] |
| | 5-chloro-2-(1-cyclopropyl-1H-pyrazol-4-yl)- |
| | 7-(trifluoromethyl)[1,2,4]triazolo[1,5-c] |
| | quinazoline |
| | LC-MS (Method 1): Rt = 1.29 min; |
| | MS (ESIpos): m/z = 379 [M + H]⁺ |
| | ¹H-NMR (400 MHz, DMSO-d₆): δ [ppm] = |
| | 1.00-1.06 (m, 2H), 1.16-1.21 (m, 2H), 3.90 |
| | (tt, 1H), 7.98 (t, 1H), 8.11 (s, 1H), 8.35 (dd, |
| | 1H), 8.62 (s, 1H), 8.73 (dd, 1H). |
| Intermediate 510 | 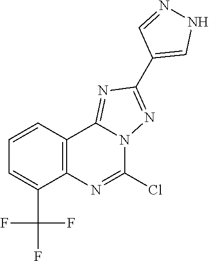 [SMILES: FC(F)(F)c1cccc2c1nc(Cl)n1nc(-c3cn[nH]c3)nc21] |
| | 5-chloro-2-(1H-pyrazol-4-yl)-7- |
| | (trifluoromethyl)[1,2,4]triazolo[1,5-c] |
| | quinazoline |
| | LC-MS (Method 1): Rt = 1.10 min; |
| | MS (ESIpos): m/z = 337 [M − H]⁺ |
| | ¹H-NMR (400 MHz, DMSO-d₆, NH-signal |
| | not seen): δ [ppm] = 7.97 (t, 1H), 8.31- |
| | 8.41 (m, 3H), 8.75 (dd, 1H). |
| Intermediate 511 | 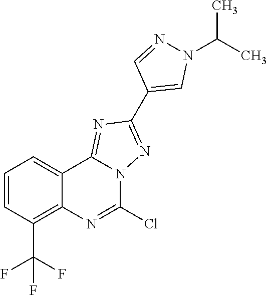 [SMILES: CC(C)n1cc(-c2nc3c4cccc(C(F)(F)F)c4nc(Cl)n3n2)cn1] |
| | 5-chloro-2-[1-(propan-2-yl)-1H-pyrazol-4-yl]- |
| | 7-(trifluoromethyl)[1,2,4]triazolo[1,5-c] |
| | quinazoline |
| | LC-MS (Method 1): Rt = 1.33 min; |
| | MS (ESIpos): m/z = 381 [M − H]⁺ |
| | ¹H-NMR (400 MHz, DMSO-d₆): δ [ppm] = |
| | 1.50 (d, 6H), 4.65 (spt, 1H), 7.97 (t, 1H), 8.13 |
| | (d, 1H), 8.35 (dd, 1H), 8.59 (d, 1H), 8.74 |
| | (dd, 1H). |
| Intermediate 512 | 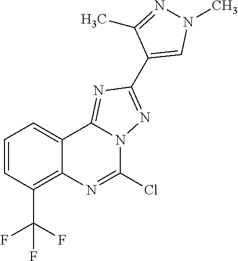 [SMILES: Cc1nn(C)cc1-c1nc2c3cccc(C(F)(F)F)c3nc(Cl)n2n1] |
| | 5-chloro-2-(1,3-dimethyl-1H-pyrazol-4-yl)-7- |
| | (trifluoromethyl)[1,2,4]triazolo[1,5-c] |
| | quinazoline |
| | LC-MS (Method 1): Rt = 1.26 min; |
| | MS (ESIpos): m/z = 367 [M + H]⁺ |
| | ¹H-NMR (400 MHz, DMSO-d₆): δ [ppm] = |
| | 2.59 (s, 3H), 3.88 (s, 3H), 7.97 (t, 1H), 8.34 |
| | (dd, 1H), 8.45 (s, 1H), 8.73 (dd, 1H). |
| Intermediate 513 | 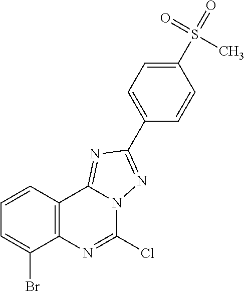 [SMILES: CS(=O)(=O)c1ccc(-c2nc3c4cccc(Br)c4nc(Cl)n3n2)cc1] |
| | 7-bromo-5-chloro-2-[4-(methanesulfonyl) |
| | phenyl][1,2,4]triazolo[1,5-c]quinazoline |
| | LC-MS (method 2): Rₜ = 1.24 min; |
| | MS (ESIpos): m/z = 437 [M]⁺ |
| Intermediate 514 | 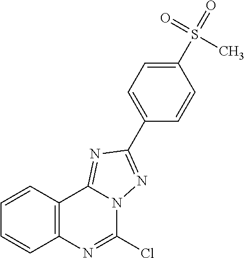 [SMILES: CS(=O)(=O)c1ccc(-c2nc3c4ccccc4nc(Cl)n3n2)cc1] |
| | 5-chloro-2-[4-(methanesulfonyl)phenyl][1,2,4] |
| | triazolo[1,5-c]quinazoline |
| | LC-MS (method 2): Rₜ = 1.13 min; |
| | MS (ESIpos): m/z = 359 [M + H]⁺ |
| Intermediate 515 | 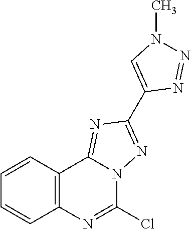 [SMILES: Cn1cc(-c2nc3c4ccccc4nc(Cl)n3n2)nn1] |
| | 5-chloro-2-(1-methyl-1H-1,2,3-triazol-4-yl) |
| | [1,2,4]triazolo[1,5-c]quinazoline |
| | LC-MS (Method 2): Rₜ = 0.88 min; |
| | MS (ESIpos): m/z = 286 [M + H]⁺ |
| | ¹H-NMR (400 MHz, DMSO-d₆): δ [ppm] = |
| | 4.18 (s, 3H), 7.85-7.91 (m, 1H), 7.99 (td, 1H), |
| | 8.05 (d, 1H), 8.49 (d, 1H), 8.91 (s, 1H). |
| Intermediate 516 | 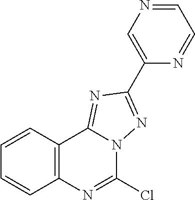 [SMILES: Clc1nc2ccccc2c2nc(-c3cnccn3)nn12] |
| | 5-chloro-2-(pyrazin-2-yl)[1,2,4]triazolo |
| | [1,5-c]quinazoline |
| | LC-MS (Method 2): Rₜ = 0.96 min; |
| | MS (ESIpos): m/z = 283 [M + H]⁺ |
| | ¹H-NMR (400 MHz, DMSO-d₆): δ [ppm] = |
| | 7.91 (ddd, 1H), 7.99-8.04 (m, 1H), 8.07-8.10 |
| | (m, 1H), 8.57 (dd, 1H), 8.88 (d, 1H), 8.91 |
| | (dd, 1H), 9.54 (d, 1H). |
| Intermediate 517 | 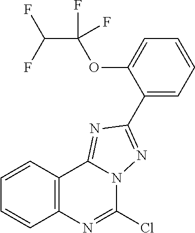 [SMILES: FC(F)C(F)(F)Oc1ccccc1-c1nc2c3ccccc3nc(Cl)n2n1] |
| | 5-chloro-2-[2-(1,1,2,2-tetrafluoroethoxy) |
| | phenyl][1,2,4]triazolo[1,5-c]quinazoline |
| | LC-MS (Method 2): Rₜ = 1.47 min; |
| | MS (ESIpos): m/z = 397 [M + H]⁺ |
| | ¹H-NMR (400 MHz, DMSO-d₆): δ [ppm] = |
| | 6.81 (tt, 1H), 7.59 (dd, 1H), 7.63 (td, 1H), |
| | 7.69-7.75 (m, 1H), 7.89 (ddd, 1H), 8.00 |
| | (ddd, 1H), 8.05-8.09 (m, 1H), 8.40 (dd, 1H), |
| | 8.54 (dd, 1H). |
| Intermediate 518 | 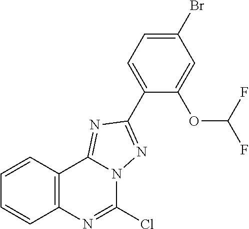 [SMILES: FC(F)Oc1cc(Br)ccc1-c1nc2c3ccccc3nc(Cl)n2n1] |
| | 2-[4-bromo-2-(difluoromethoxy)phenyl]-5- |
| | chloro[1,2,4]triazolo[1,5-c]quinazoline |
| | LC-MS (Method 2): Rₜ = 1.49 min; |
| | MS (ESIpos): m/z = 425 [M + H]⁺ |
| | ¹H-NMR (400 MHz, DMSO-d₆): δ [ppm] = |
| | 7.40 (t, 2H), 7.69 (s, 1H), 7.75 (dd, 1H), 7.86- |
| | 7.92 (m, 1H), 7.97-8.03 (m, 1H), 8.07 (d, 1H), |
| | 8.22 (d, 1H), 8.51 (d, 1H). |

### Intermediate 519

### benzyl (6R)-6-{[2-(5-bromo-2-furyl)[1,2,4]triazolo[1,5-c]quinazolin-5-yl]amino}-5-oxo-1,4-di azepane-1-carboxylate

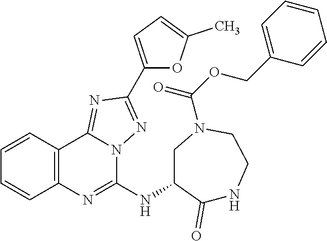 [SMILES: Cc1ccc(-c2nc3c4ccccc4nc(N[C@@H]4CN(C(=O)OCc5ccccc5)CCNC4=O)n3n2)o1]

2-(5-bromofuran-2-yl)-5-chloro[1,2,4]triazolo[1,5-c]quinazoline (100 mg, 286 μmol), benzyl (6R)-6-amino-5-oxo-1,4-diazepane-1-carboxylate hydrochloride (94.3 mg, 315 μmol) and N,N-diisopropylethylamine (200 μl, 1.1 mmol) were stirred in DMSO (2.0 mL) for 2 h at 60° C. Water was added to the mixture, filtered, washed with water and dried under reduced pressure at 60° C. to give 144 mg (97% purity, 85% yield) of the target compound.

LC-MS (Method 2): Rₜ=1.33 min; MS (ESIpos): m/z=576 [M+H]⁺

¹H-NMR (400 MHz, DMSO-d₆): δ [ppm]=2.93-3.13 (m, 1H), 3.21-3.32 (m, 2H), 3.44-3.54 (m, 1H), 4.10-4.27 (m, 1H), 4.52-4.66 (m, 1H), 4.87-5.00 (m, 1H), 5.19 (s, 2H), 6.90 (d, 1H), 7.13-7.27 (m, 2H), 7.27-7.52 (m, 6H), 7.59-7.71 (m, 1H), 7.88 (br d, 1H), 8.27 (br d, 1H), 8.35-8.53 (m, 1H).

The following intermediates were prepared following the same procedure as described for intermediate 519 (in some cases preparative PLC was used for purification):

| | Structure |
|---|---|
| | IUPAC-Name |
| | LC-MS (method): Retention time; Mass found |
| Intermediate | ¹H-NMR |
| Intermediate 521 | 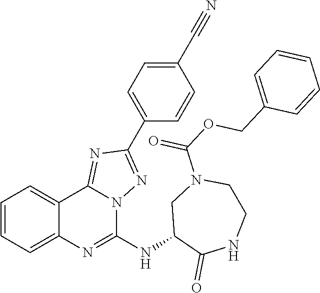 [SMILES: N#Cc1ccc(-c2nc3c4ccccc4nc(N[C@@H]4CN(C(=O)OCc5ccccc5)CCNC4=O)n3n2)cc1] |
| | benzyl (6R)-6-{[2-(4-cyanophenyl) |
| | [1,2,4]triazolo[1,5-c]quinazolin-5- |
| | yl]amino}5-oxo-1,4-diazepane-1-carboxylate |
| | LC-MS (Method 2): Rt = 1.34 min; |
| | MS (ESIpos): m/z = 533 [M + H]⁺ |
| | ¹H-NMR (400 MHz, DMSO-d₆): δ |
| | [ppm] = 2.94-3.13 (m, 1H), 3.41-3.56 (m, |
| | 2H and water signal), 4.12-4.28 |
| | (m, 1H), 4.56-4.70 (m, 1H), 4.95 (br s, |
| | 1H), 5.20 (br s, 2H), 7.19 (br s, 1.5H, |
| | rotamers), 7.29-7.53 (m, 5H), 7.60- |
| | 7.84 (m, 1.5H, rotamers), 7.94 (br d, 1H), |
| | 8.08 (br d, 2H), 8.32 (brd, 1H), |
| | 8.39-8.55 (m, 3H). |
| Intermediate 522 | 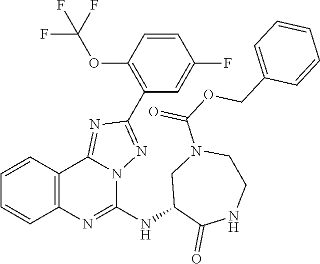 [SMILES: O=C1NCCN(C(=O)OCc2ccccc2)C[C@H]1Nc1nc2ccccc2c2nc(-c3cc(F)ccc3OC(F)(F)F)nn12] |
| | benzyl (6R)-6-({2-[5-fluoro-2- |
| | (trifluoromethoxy)phenyl][1,2,4] |
| | triazolo[1,5-c]quinazolin-5-yl}amino)-5- |
| | oxo-1,4-diazepane-1-carboxylate |
| | LC-MS (Method 2): Rt = 1.50 min; |
| | MS (ESIpos): m/z = 610 [M + H]⁺ |
| | ¹H-NMR (400 MHz, DMSO-d₆): δ |
| | [ppm] = 2.90-3.11 (m, 1H), 4.13-4.29 (m, |
| | 1H), 4.63-4.79 (m, 1H), 4.88-4.98 (m, 1H), |
| | 5.21 (br s, 2H), 7.11-7.92 (m, |
| | 11H), 8.14 (br d. 1H), 8.30 (br d, 1H), |
| | 8.37-8.56 (m, 1H). |
| Intermediate 523 | 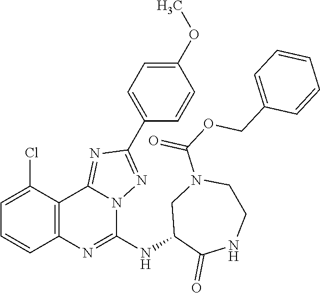 [SMILES: COc1ccc(-c2nc3c4c(Cl)cccc4nc(N[C@@H]4CN(C(=O)OCc5ccccc5)CCNC4=O)n3n2)cc1] |
| | benzyl (6R)-6-{[10-chloro-2-(4- |
| | methoxyphenyl)[1,2,4]triazolo[1,5- |
| | c]quinazolin-5-yl]amino}-5-oxo-1,4- |
| | diazepane-1-carboxylate |
| | LC-MS (Method 2): Rₜ = 1.46 min; |
| | MS (ESIpos): m/z = 572 [M + H]⁺ |
| | ¹H-NMR (400 MHz, DMSO-d₆): δ |
| | [ppm] = 2.93-3.13 (m, 1H), 3.44-3.54 (m, |
| | 1H), 3.86 (s, 3H), 4.12-4.27 (m, 1H), |
| | 4.55-4.72 (m, 1H), 4.93 (brs, 1H), |
| | 5.20 (br s, 2H), 7.13-7.18 (m, 2H), |
| | 7.18-7.73 (m, 8H), 7.97 (d, 1H), 8.23 |
| | (br d, 2H), 8.46 (br d, 1H). |
| Intermediate 524 | 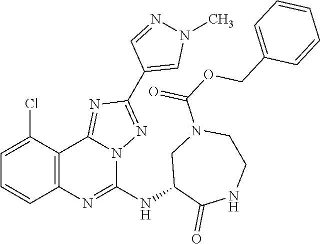 [SMILES: Cn1cc(-c2nc3c4c(Cl)cccc4nc(N[C@@H]4CN(C(=O)OCc5ccccc5)CCNC4=O)n3n2)cn1] |
| | benzyl (6R)-6-{[10-chloro-2-(1-methyl- |
| | 1H-pyrazol-4-yl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.23 min; |
| | MS (ESIpos): m/z = 546 [M + H]⁺ |
| | ¹H-NMR (400 MHz, DMSO-d₆): δ |
| | [ppm] = 2.92-3.11 (m, 1H), 3.12-3.32 (m, |
| | 2H), 3.44-3.54 (m, 1H), 3.96 (s, 3H), |
| | 4.12-4.27 (m, 1H), 4.55-4.73 (m, |
| | 1H), 4.87-4.98 (m, 1H), 5.20 (s, 2H), |
| | 7.14-7.74 (m, 8H), 7.86 (br d, 1H), |
| | 8.05 (s, 1H), 8.42-8.55 (m, 2H). |
| Intermediate 525 | 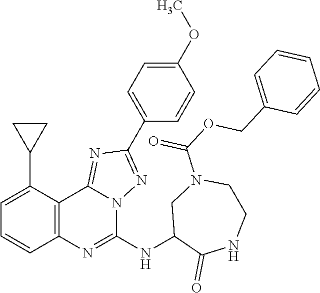 [SMILES: COc1ccc(-c2nc3c4c(C5CC5)cccc4nc(NC4CN(C(=O)OCc5ccccc5)CCNC4=O)n3n2)cc1] |
| | benzyl 6-{[10-cyclopropyl-2- |
| | (4-methoxyphenyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.55 min; |
| | MS (ESIpos): m/z = 578 [M + H]⁺ |
| | ¹H-NMR (400 MHz, DMSO-d₆): δ |
| | [ppm] = 0.82-0.91 (m, 2H), 1.18-1.26 (m, |
| | 2H), 2.93-3.13 (m, 1H), 3.16-3.32 (m, 2H), |
| | 3.45-3.55 (m, 1H), 3.79-3.88 |
| | (m, 4H), 4.11-4.27 (m, 1H), 4.56-4.72 |
| | (m, 1H), 4.87-4.99 (m, 1H), 5.19 |
| | (br s, 2H), 6.97 (br d, 1H), 7.13-7.64 (m, 9H), |
| | 7.84 (br d, 1H), 8.23 (br d, |
| | 2H), 8.38-8.53 (m, 1H). |
| Intermediate 526 | 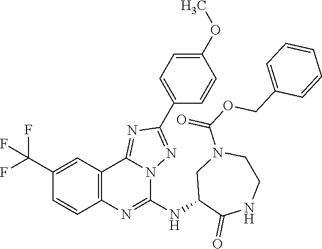 [SMILES: COc1ccc(-c2nc3c4cc(C(F)(F)F)ccc4nc(N[C@@H]4CN(C(=O)OCc5ccccc5)CCNC4=O)n3n2)cc1] |
| | benzyl (6R)-6-{[2-(4-methoxyphenyl)-9- |
| | (trifluoromethyl)[1,2,4]triazolo[1,5-c] |
| | quinazolin-5-yl]amino}-5-oxo-1,4- |
| | diazepane-1-carboxylate |
| | LC-MS (Method 2): Rₜ = 1.52 min; |
| | MS (ESIpos): m/z = 606 [M + H]⁺ |
| | ¹H-NMR (400 MHz, DMSO-d₆): δ |
| | [ppm] = 2.94-3.15 (m, 1H), 3.19-3.37 (m, |
| | 2H and water signal), 3.51 (br s, 1H), |
| | 3.86 (s, 3H), 4.12-4.28 (m, 1H), 4.56- |
| | 4.72 (m, 1H), 4.91-5.04 (m, 1H), 5.20 |
| | (br s, 2H), 7.11-7.47 (m, 7H), 7.49- |
| | 7.56 (m, 0.5H, rotamer), 7.75-7.90 |
| | (m, 1H), 8.00-8.07 (m, 0.5H, |
| | rotamer), 8.08-8.18 (m, 1H), |
| | 8.24 (br d, 2H), 8.40-8.57 (m, 2H). |
| Intermediate 527 | 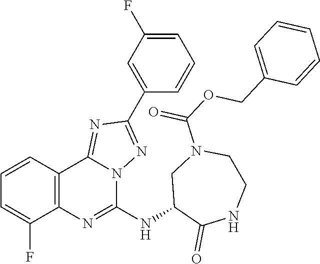 [SMILES: O=C1NCCN(C(=O)OCc2ccccc2)C[C@H]1Nc1nc2c(F)cccc2c2nc(-c3cccc(F)c3)nn12] |
| | benzyl (6R)-6-{[7-fluoro-2-(3- |
| | fluorophenyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.40 min; |
| | MS (ESIpos): m/z = 544 [M + H]⁺ |
| | ¹H-NMR (400 MHz, DMSO-d₆): δ |
| | [ppm] = 2.96-3.20 (m, 1H), 3.28-3.53 (m, |
| | 3H and water signal), 4.04-4.23 (m, 1H), |
| | 4.50 (br d, 1H), 4.90-5.29 (m, |
| | 3H), 7.03-7.48 (m, 7H), 7.54-7.70 (m, 2H), |
| | 8.00 (br d, 1H), 8.09-8.51 (m, 4H). |
| Intermediate 528 | 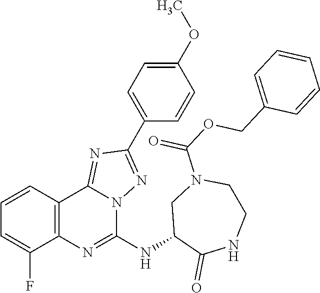 [SMILES: COc1ccc(-c2nc3c4cccc(F)c4nc(N[C@@H]4CN(C(=O)OCc5ccccc5)CCNC4=O)n3n2)cc1] |
| | benzyl (6R)-6-{[7-fluoro-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.35 min; |
| | MS (ESIpos): m/z = 556 [M + H]⁺ |
| | ¹H-NMR (400 MHz, DMSO-d₆): δ |
| | [ppm] = 2.94-3.20 (m, 1H), 3.28-3.53 (m, |
| | 3H and water signal), 3.86 (s, 3H), 4.06-4.22 |
| | (m, 1H), 4.50 (br d, 1H), 4.91- |
| | 5.29 (m, 3H), 7.04-7.18 (m, 4H), 7.22-7.47 |
| | (m, 4H), 7.51-7.64 (m, 1H), |
| | 8.01-8.19 (m, 2H), 8.23 (br d, 2H), |
| | 8.30-8.50 (m, 1H). |
| Intermediate 529 | 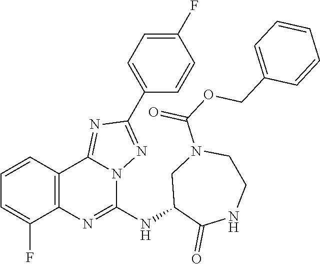 [SMILES: O=C1NCCN(C(=O)OCc2ccccc2)C[C@H]1Nc1nc2c(F)cccc2c2nc(-c3ccc(F)cc3)nn12] |
| | benzyl (6R)-6-{[7-fluoro-2-(4-fluorophenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.39 min; |
| | MS (ESIpos): m/z = 544 [M + H]⁺ |
| | ¹H-NMR (400 MHz, DMSO-d₆): δ |
| | [ppm] = 2.96-3.20 (m, 1H), 3.28-3.54 (m, |
| | 3H and water signal), 4.02-4.23 (m, 1H), |
| | 4.51 (br d, 1H), 4.89-5.29 (m, |
| | 3H), 7.02-7.48 (m, 8H), 7.52-7.65 (m, 1H), |
| | 8.03-8.25 (m, 2H), 8.28-8.52 (m, 3H). |
| Intermediate 530 | 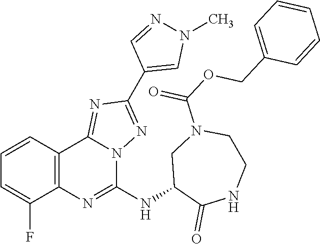 [SMILES: Cn1cc(-c2nc3c4cccc(F)c4nc(N[C@@H]4CN(C(=O)OCc5ccccc5)CCNC4=O)n3n2)cn1] |
| | benzyl (6R)-6-{[7-fluoro-2-(1-methyl- |
| | 1H-pyrazol-4-yl)[1,2,4]triazolo[1,5- |
| | c]quinazolin-5-yl]amino}-5-oxo- |
| | 1,4-diazepane-1-carboxylate |
| | LC-MS (Method 2): Rt = 1.13 min; |
| | MS (ESIpos): m/z = 530 [M + H]⁺ |
| Intermediate 531 | 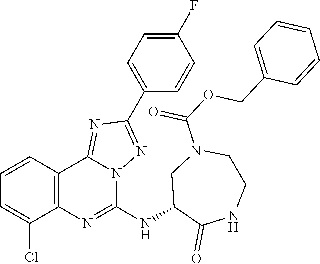 [SMILES: O=C1NCCN(C(=O)OCc2ccccc2)C[C@H]1Nc1nc2c(Cl)cccc2c2nc(-c3ccc(F)cc3)nn12] |
| | benzyl (6R)-6-{[7-chloro-2-(4- |
| | fluorophenyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.46 min; |
| | MS (ESIpos): m/z = 560 [M + H]⁺ |
| | ¹H-NMR (400 MHz, DMSO-d₆): δ |
| | [ppm] = 3.00-3.21 (m, 1H), 3.36-3.58 (m, |
| | 3H), 4.12 (br d, 1H), 4.42 (br d, 1H), |
| | 4.91-5.21 (m, 3H), 7.00-7.46 (m, 8H), |
| | 7.87 (br d, 1H), 8.16-8.46 (m, 5H). |
| Intermediate 532 | 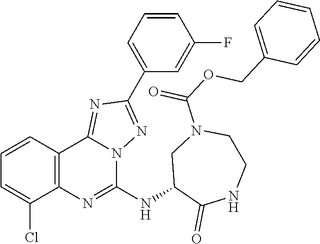 [SMILES: O=C1NCCN(C(=O)OCc2ccccc2)C[C@H]1Nc1nc2c(Cl)cccc2c2nc(-c3cccc(F)c3)nn12] |
| | benzyl (6R)-6-{[7-chloro-2-(3- |
| | fluorophenyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.46 min; |
| | MS (ESIpos): m/z = 560 [M + H]⁺ |
| | ¹H-NMR (400 MHz, DMSO-d₆): δ |
| | [ppm] = 3.02-3.22 (m, 1H), 3.38-3.59 (m, |
| | 3H), 4.12 (br d, 1H), 4.36-4.52 (m, 1H), |
| | 4.91-5.21 (m, 3H), 6.98-7.47 (m, 7H), |
| | 7.66 (td, 1H), 7.87 (brd, 1H), 7.99 (brd, 1H), |
| | 8.13 (d, 1H), 8.22-8.45 (m, 3H). |
| Intermediate 533 | 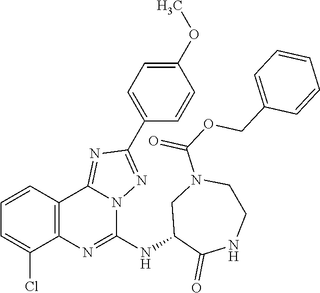 [SMILES: COc1ccc(-c2nc3c4cccc(Cl)c4nc(N[C@@H]4CN(C(=O)OCc5ccccc5)CCNC4=O)n3n2)cc1] |
| | benzyl (6R)-6-{[7-chloro-2-(4-methoxyphenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.39 min; |
| | MS (ESIpos): m/z = 572 [M + H]⁺ |
| | ¹H-NMR (400 MHz, DMSO-d₆): δ |
| | [ppm] = 3.03-3.20 (m, 1H), 3.39-3.57 (m, |
| | 2H), 3.86 (s, 3H), 4.07-4.16 (m, 1H), |
| | 4.37-4.50 (m, 1H), 4.91-5.21 (m, |
| | 3H), 6.98-7.18 (m, 4H), 7.19-7.45 (m, 4H), |
| | 7.87 (br d, 1H), 8.13-8.29 (m, |
| | 4H), 8.31-8.43 (m, 1H). |
| Intermediate 534 | 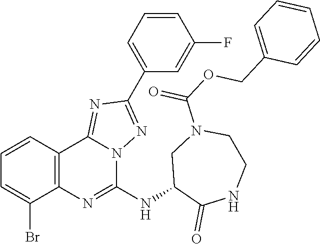 [SMILES: O=C1NCCN(C(=O)OCc2ccccc2)C[C@H]1Nc1nc2c(Br)cccc2c2nc(-c3cccc(F)c3)nn12] |
| | benzyl (6R)-6-{[7-bromo-2-(3-fluorophenyl) |
| | [1,2,4]triazolo[1,5- |
| | c]quinazolin-5-yl]amino}-5-oxo- |
| | 1,4-diazepane-1-carboxylate |
| | LC-MS (Method 2): Rₜ = 1.47 min; |
| | MS (ESIpos): m/z = 604 [M + H]⁺ |
| | ¹H-NMR (400 MHz, DMSO-d₆): δ |
| | [ppm] = 3.05-3.24 (m, 1H), 3.36-3.47 (m, |
| | 2H), 3.49- 3.64 (m, 1H), 4.07-4.17 (m, 1H), |
| | 4.30-4.50 (m, 1H), 4.93-5.20 |
| | (m, 3H), 6.98-7.48 (m, 7H), 7.67 (td, 1H), |
| | 8.02 (dt, 1H), 8.06 (dd, 1H), 8.15 |
| | (d, 1H), 8.28-8.43 (m, 3H). |
| Intermediate 535 | 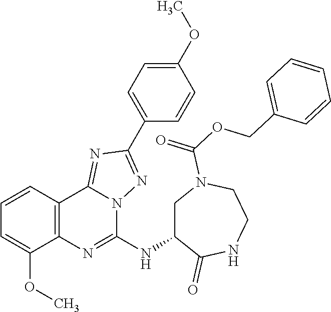 [SMILES: COc1ccc(-c2nc3c4cccc(OC)c4nc(N[C@@H]4CN(C(=O)OCc5ccccc5)CCNC4=O)n3n2)cc1] |
| | benzyl (6R)-6-{[7-methoxy-2-(4- |
| | methoxyphenyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.29 min; |
| | MS (ESIpos): m/z = 568 [M + H]⁺ |
| | ¹H-NMR (400 MHz, DMSO-d₆): δ |
| | [ppm] = 3.00-3.23 (m, 1H), 3.39-3.52 (m, |
| | 3H), 3.79-3.97 (m, 6H), 4.01-4.17 (m, 1H), |
| | 4.40 (dd, 1H), 4.85-5.24 (m, |
| | 3H), 7.00-7.18 (m, 4H), 7.21-7.46 (m, 5H), |
| | 7.88 (dd, 2H), 8.20-8.42 (m, 3H). |
| Intermediate 536 | 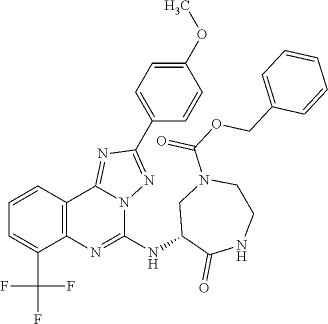 [SMILES: COc1ccc(-c2nc3c4cccc(C(F)(F)F)c4nc(N[C@@H]4CN(C(=O)OCc5ccccc5)CCNC4=O)n3n2)cc1] |
| | benzyl (6R)-6-{[2-(4-methoxyphenyl)-7- |
| | (trifluoromethyl)[1,2,4]triazolo[1,5-c] |
| | quinazolin-5-yl]amino}-5-oxo-1,4- |
| | diazepane-1-carboxylate |
| | LC-MS (Method 2): Rₜ = 1.41 min; |
| | MS (ESIneg): m/z = 604 [M − H]⁻ |
| | ¹H-NMR (400 MHz, DMSO-d₆): δ |
| | [ppm] = 3.11-3.24 (m, 1H), 3.48-3.72 (m, |
| | 1H), 3.86 (s, 3H), 4.00-4.09 (m, 1H), |
| | 4.19-4.37 (m, 1H), 4.90-5.21 (m, |
| | 3H), 6.99-7.45 (m, 7H), 7.56 (t, 1H), 8.08 (d, 1H), |
| | 8.22-8.32 (m, 3H), 8.47-8.59 (m, 2H). |
| Intermediate 537 | 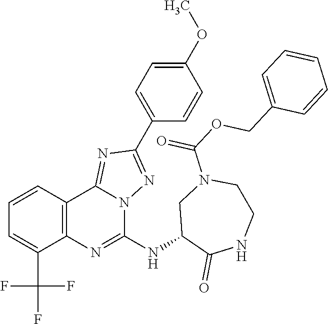 [SMILES: COc1ccc(-c2nc3c4cccc(C(F)(F)F)c4nc(N[C@@H]4CN(C(=O)OCc5ccccc5)CCNC4=O)n3n2)cc1] |
| | benzyl (6R)-6-{[2-(4-methoxyphenyl)-7- |
| | (trifluoromethyl)[1,2,4]triazolo[1,5-c] |
| | quinazolin-5-yl]amino}-5-oxo-1,4- |
| | diazepane-1-carboxylate |
| | LC-MS (Method 2): Rₜ = 1.43 min; |
| | MS (ESIpos): m/z = 606 [M + H]⁺ |
| | ¹H-NMR (400 MHz, DMSO-d₆): δ |
| | [ppm] = 3.12-3.22 (m, 1H), 3.49-3.71 (m, |
| | 1H), 3.87 (s, 3H), 4.01-4.09 (m, 1H), |
| | 4.20-4.37 (m, 1H), 4.88-5.20 (m, |
| | 3H), 6.99-7.46 (m, 7H), 7.56 (t, 1H), |
| | 8.08 (d, 1H), 8.25 (br d, 3H), 8.46- |
| | 8.59 (m, 2H). |
| Intermediate 538 | 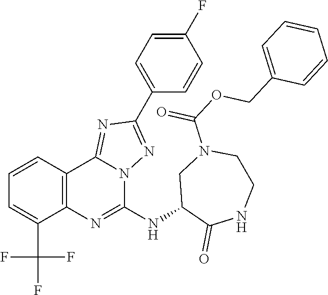 [SMILES: O=C1NCCN(C(=O)OCc2ccccc2)C[C@H]1Nc1nc2c(C(F)(F)F)cccc2c2nc(-c3ccc(F)cc3)nn12] |
| | benzyl (6R)-6-{[2-(4-fluorophenyl)-7- |
| | (trifluoromethyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.46 min; |
| | MS (ESIpos): m/z = 594 [M + H]⁺ |
| | ¹H-NMR (400 MHz, DMSO-d₆): δ |
| | [ppm] = 3.12-3.22 (m, 1H), |
| | 3.49-3.72 (m, 1H), |
| | 4.05 (br d, 1H), 4.20-4.37 (m, 1H), |
| | 4.89-5.20 (m, 3H), 6.98-7.42 (m. |
| | 5H), 7.43-7.50 (m, 2H), 7.57 (t, 1H), |
| | 8.10 (d, 1H), 8.22-8.32 (m, 1H), 8.36 |
| | (dd, 2H), 8.52-8.61 (m, 2H). |
| Intermediate 539 | 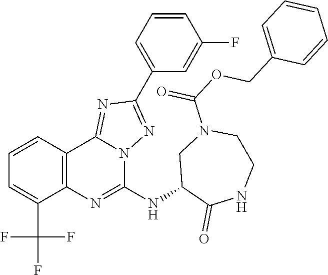 [SMILES: O=C1NCCN(C(=O)OCc2ccccc2)C[C@H]1Nc1nc2c(C(F)(F)F)cccc2c2nc(-c3cccc(F)c3)nn12] |
| | benzyl (6R)-6-{[2-(3-fluorophenyl)-7- |
| | (trifluoromethyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.47 min; |
| | MS (ESIpos): m/z = 594 [M + H]⁺ |
| | ¹H-NMR (400 MHz, DMSO-d₆): δ |
| | [ppm] = 3.11-3.23 (m, 1H), 3.51-3.72 (m, |
| | 1H), 4.06 (br d, 1H), 4.19-4.38 (m, 1H), |
| | 4.89-5.20 (m, 3H), 6.99-7.48 (m, |
| | 6H), 7.58 (t, 1H), 7.68 (td, 1H), 8.03 (br d, 1H), |
| | 8.10 (d, 1H), 8.16 (d, 1H), |
| | 8.27 (br d, 1H), 8.58 (br dd, 2H). |
| Intermediate 540 | 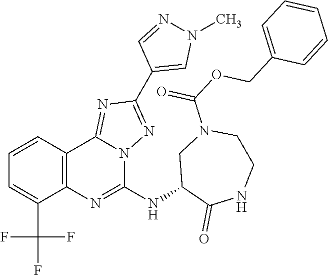 [SMILES: Cn1cc(-c2nc3c4cccc(C(F)(F)F)c4nc(N[C@@H]4CN(C(=O)OCc5ccccc5)CCNC4=O)n3n2)cn1] |
| | benzyl (6R)-6-{[2-(1-methyl- |
| | 1H-pyrazol-4-yl)-7- |
| | (trifluoromethyl)[1,2,4]triazolo[1,5-c] |
| | quinazolin-5-yl]amino}-5-oxo-1,4- |
| | diazepane-1-carboxylate |
| | LC-MS (Method 2): Rₜ = 1.19 min; |
| | MS (ESIpos): m/z = 580 [M + H]⁺ |
| | ¹H-NMR (400 MHz, DMSO-d₆): δ |
| | [ppm] = 3.11-3.22 (m, 1H), 3.47-3.70 (m, |
| | 1H), 3.97 (s, 3H), 4.00-4.07 (m, 1H), |
| | 4.20-4.33 (m, 1H), 4.90-5.20 (m, |
| | 3H), 6.97-7.45 (m, 5H), 7.55 (t, 1H), |
| | 8.06-8.11 (m, 2H), 8.22-8.31 (m, |
| | 1H), 8.35-8.43 (m, 1H), 8.47-8.54 (m, 2H). |
| Intermediate 541 | 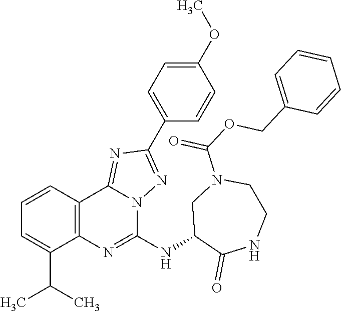 [SMILES: COc1ccc(-c2nc3c4cccc(C(C)C)c4nc(N[C@@H]4CN(C(=O)OCc5ccccc5)CCNC4=O)n3n2)cc1] |
| | benzyl (6R)-6-{[2-(4-methoxyphenyl)- |
| | 7-(propan-2-yl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.52 min; |
| | MS (ESIpos): m/z = 580 [M + H]⁺ |
| | ¹H-NMR (400 MHz, DMSO-d₆): δ |
| | [ppm] = 1.19-1.31 (m, 6H), 3.06-3.17 (m, |
| | 1H), 3.38-3.64 (m, 2H), 3.81-4.24 (m, 5H), |
| | 4.30-4.62 (m, 1H), 4.88-5.23 |
| | (m, 3H), 7.00-7.46 (m, 8H), 7.58-7.66 |
| | (m, 1H), 7.87-8.07 (m, 1H), 8.16 |
| | (dd, 1H), 8.21-8.27 (m, 2H), 8.31-8.46 (m, 1H). |
| Intermediate 542 | 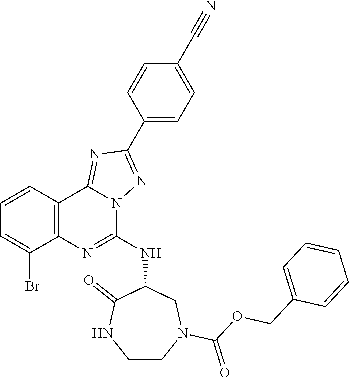 [SMILES: N#Cc1ccc(-c2nc3c4cccc(Br)c4nc(N[C@@H]4CN(C(=O)OCc5ccccc5)CCNC4=O)n3n2)cc1] |
| | benzyl (6R)-6-{[7-bromo-2-(4- |
| | cyanophenyl)[1,2,4]triazolo[1,5- |
| | c]quinazolin-5-yl]amino}-5-oxo- |
| | 1,4-diazepane-1-carboxylate |
| | LC-MS (Method 2): Rₜ = 1.37 min; |
| | MS (ESIpos): m/z = 611 [M + H]⁺ |
| | ¹H-NMR (400 MHz, DMSO-d₆): δ |
| | [ppm] = 3.05-3.23 (m, 1H), 3.38-3.64 (m, |
| | 3H), 4.12 (br d, 1H), 4.33-4.49 (m, 1H), |
| | 4.91-5.22 (m, 3H), 6.97-7.45 (m, |
| | 6H), 8.04-8.11 (m, 3H), 8.29-8.48 (m, 5H). |

### Intermediate 543

### benzyl (6R)-6-{[2-(3,4-dimethoxyphenyl)[1,2,4]triazolo[1,5-c]quinazolin-5-yl]amino}-5-oxo-1,4-diazepane-1-carboxylate

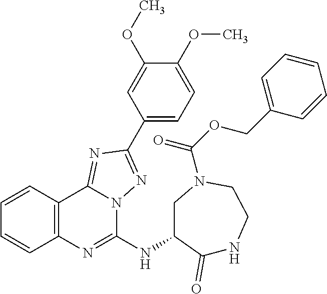 [SMILES: COc1ccc(-c2nc3c4ccccc4nc(N[C@@H]4CN(C(=O)OCc5ccccc5)CCNC4=O)n3n2)cc1OC]

Benzyl (6R)-6-[(2-bromo[1,2,4]triazolo[1,5-c]quinazolin-5-yl)amino]-5-oxo-1,4-diazepane-1-carboxylate (100 mg, 196 μmol), 2-(3,4-dimethoxyphenyl)-4,4,5,5-tetramethyl-1,3,2-dioxaborolane (104 mg, 392 μmol) and XPhos Pd G4 (8.43 mg, 9.80 μmol) were dissolved in DMF (1.5 mL), treated with potassium carbonate solution (290 μL, 2.0 M, 590 μmol) and stirred overnight at 90° C. The mixture was poured into water and the precipitate was filtered, washed with water and dried under reduced pressure to give the title compound (111.2 mg). The residue was used without further purification.

LC-MS (Method 2): Rt=1.27 min; MS (ESIpos): m/z=568 [M+H]⁺

The following intermediates were prepared similarly to intermediate 543:

| | Structure |
|---|---|
| | IUPAC-Name |
| | LC-MS (method): Retention time; Mass found |
| Intermediate | ¹H-NMR |
| Intermediate 544 | 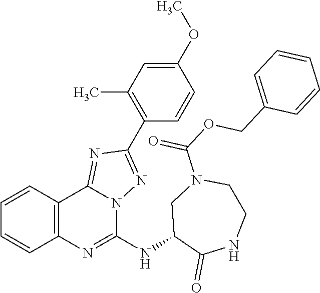 [SMILES: COc1ccc(-c2nc3c4ccccc4nc(N[C@@H]4CN(C(=O)OCc5ccccc5)CCNC4=O)n3n2)c(C)c1] |
| | benzyl (6R)-6-{[2-(4-methoxy-2-methylphenyl)[1,2,4]triazolo[1,5- |
| | c]quinazolin-5-yl]amino}-5-oxo-1,4-diazepane-1-carboxylate |
| | LC-MS (Method 2): Rt = 1.40 min; MS (ESIpos): m/z = 552 [M + H]⁺ |
| Intermediate 545 | 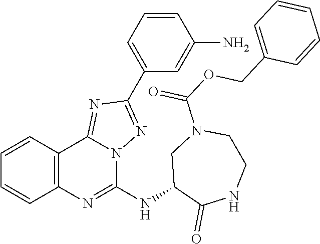 [SMILES: Nc1cccc(-c2nc3c4ccccc4nc(N[C@@H]4CN(C(=O)OCc5ccccc5)CCNC4=O)n3n2)c1] |
| | benzyl (6R)-6-{[2-(3-aminophenyl)[1,2,4]triazolo[1,5-c]quinazolin-5- |
| | yl]amino}-5-oxo-1,4-diazepane-1-carboxylate |
| | LC-MS (Method 2): Rt = 1.18 min; MS (ESIpos): m/z = 523 [M + H]⁺ |
| Intermediate 546 | 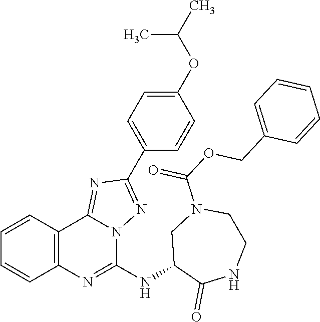 [SMILES: CC(C)Oc1ccc(-c2nc3c4ccccc4nc(N[C@@H]4CN(C(=O)OCc5ccccc5)CCNC4=O)n3n2)cc1] |
| | benzyl (6R)-5-oxo-6-[(2-{4-[(propan-2-yl)oxy]phenyl}[1,2,4]triazolo[1,5- |
| | c]quinazolin-5-yl)amino]-1,4-diazepane-1-carboxylate |
| | LC-MS (Method 2): Rt = 1.46 min; MS (ESIpos): m/z = 566 [M + H]⁺ |
| Intermediate 547 | 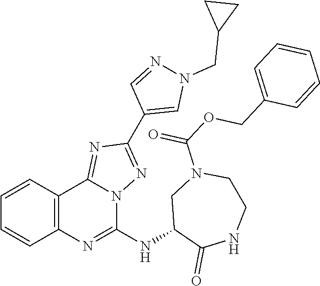 [SMILES: O=C1NCCN(C(=O)OCc2ccccc2)C[C@H]1Nc1nc2ccccc2c2nc(-c3cnn(CC4CC4)c3)nn12] |
| | benzyl (6R)-6-({2-[1-(cyclopropylmethyl)-1H-pyrazol-4- |
| | yl][1,2,4]triazolo[1,5-c]quinazolin-5-yl}amino)-5-oxo-1,4-diazepane-1- |
| | carboxylate |
| | LC-MS (Method 2): Rt = 1.22 min; MS (ESIpos): m/z = 552 [M + H]⁺ |
| Intermediate 548 | 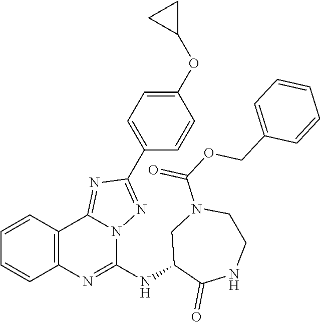 [SMILES: O=C1NCCN(C(=O)OCc2ccccc2)C[C@H]1Nc1nc2ccccc2c2nc(-c3ccc(OC4CC4)cc3)nn12] |
| | benzyl (6R)-6-({2-[4-(cyclopropyloxy)phenyl][1,2,4]triazolo[1,5- |
| | c]quinazolin-5-yl}amino)-5-oxo-1,4-diazepane-1-carboxylate |
| | LC-MS (Method 2): Rt = 1.43 min; MS (ESIpos): m/z = 564 [M + H]⁺ |
| Intermediate 549 | 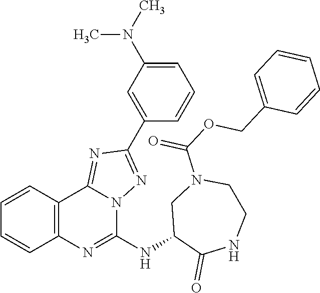 [SMILES: CN(C)c1cccc(-c2nc3c4ccccc4nc(N[C@@H]4CN(C(=O)OCc5ccccc5)CCNC4=O)n3n2)c1] |
| | benzyl (6R)-6-({2-[3-(dimethylamino)phenyl][1,2,4]triazolo[1,5- |
| | c]quinazolin-5-yl}amino)-5-oxo-1,4-diazepane-1-carboxylate |
| | LC-MS (Method 2): Rt = 1.39 min; MS (ESIpos): m/z = 551 [M + H]⁺ |
| Intermediate 550 | 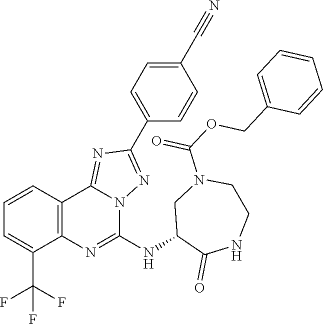 [SMILES: N#Cc1ccc(-c2nc3c4cccc(C(F)(F)F)c4nc(N[C@@H]4CN(C(=O)OCc5ccccc5)CCNC4=O)n3n2)cc1] |
| | benzyl (6R)-6-{[2-(4-cyanophenyl)-7-(trifluoromethyl)[1,2,4]triazolo[1,5- |
| | c]quinazolin-5-yl]amino}-5-oxo-1,4-diazepane-1-carboxylate |
| | LC-MS (Method 2): Rt = 1.42 min; MS (ESIpos): m/z = 601 [M + H]⁺ |
| Intermediate 551 | 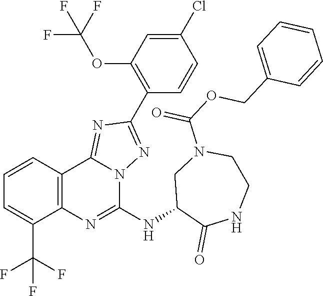 [SMILES: O=C1NCCN(C(=O)OCc2ccccc2)C[C@H]1Nc1nc2c(C(F)(F)F)cccc2c2nc(-c3ccc(Cl)cc3OC(F)(F)F)nn12] |
| | benzyl (6R)-6-({2-[4-chloro-2-(trifluoromethoxy)phenyl]-7- |
| | (trifluoromethyl)[1,2,4]triazolo[1,5-c]quinazolin-5-yl}amino)-5-oxo-1,4- |
| | diazepane-1-carboxylate |
| | LC-MS (Method 1): Rt = 1.62 min; MS (ESIpos): m/z = 692 [M − H]⁺ |
| Intermediate 552 | 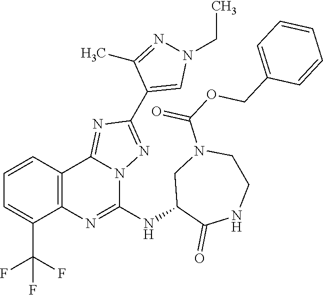 [SMILES: CCn1cc(-c2nc3c4cccc(C(F)(F)F)c4nc(N[C@@H]4CN(C(=O)OCc5ccccc5)CCNC4=O)n3n2)c(C)n1] |
| | benzyl (6R)-6-{[2-(1-ethyl-3-methyl-1H-pyrazol-4-yl)-7- |
| | (trifluoromethyl)[1,2,4]triazolo[1,5-c]quinazolin-5-yl]amino}-5-oxo-1,4- |
| | diazepane-1-carboxylate |
| | LC-MS (Method 1): Rt = 1.33 min; MS (ESIpos): m/z = 608 [M + H]⁺ |
| Intermediate 553 | 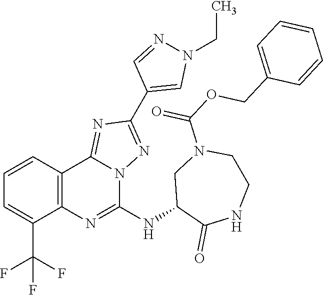 [SMILES: CCn1cc(-c2nc3c4cccc(C(F)(F)F)c4nc(N[C@@H]4CN(C(=O)OCc5ccccc5)CCNC4=O)n3n2)cn1] |
| | benzyl (6R)-6-{[2-(1-ethyl-1H-pyrazol-4-yl)-7- |
| | (trifluoromethyl)[1,2,4]triazolo[1,5-c]quinazolin-5-yl]amino}-5-oxo-1,4- |
| | diazepane-1-carboxylate |
| | LC-MS (Method 1): Rt = 1.28 min; MS (ESIpos): m/z = 594 [M + H]⁺ |
| Intermediate 554 | 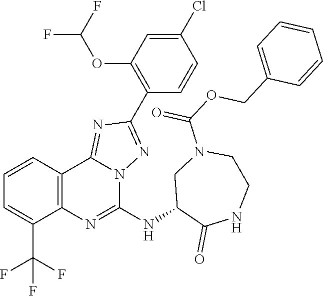 [SMILES: O=C1NCCN(C(=O)OCc2ccccc2)C[C@H]1Nc1nc2c(C(F)(F)F)cccc2c2nc(-c3ccc(Cl)cc3OC(F)F)nn12] |
| | benzyl (6R)-6-({2-[4-chloro-2-(difluoromethoxy)phenyl]-7- |
| | (trifluoromethyl)[1,2,4]triazolo[1,5-c]quinazolin-5-yl}amino)-5-oxo-1,4- |
| | diazepane-1-carboxylate |
| | LC-MS (Method 1): Rt = 1.56 min; MS (ESIpos): m/z = 676 [M + H]⁺ |
| Intermediate 555 | 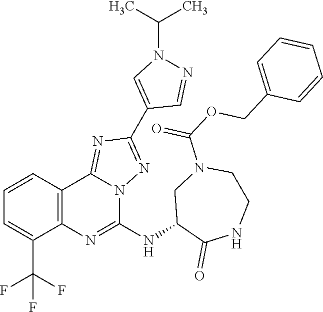 [SMILES: CC(C)n1cc(-c2nc3c4cccc(C(F)(F)F)c4nc(N[C@@H]4CN(C(=O)OCc5ccccc5)CCNC4=O)n3n2)cn1] |
| | benzyl (6R)-5-oxo-6-({2-[1-(propan-2-yl)-1H-pyrazol-4-yl]-7- |
| | (trifluoromethyl)[1,2,4]triazolo[1,5-c]quinazolin-5-yl}amino)-1,4- |
| | diazepane-1-carboxylate |
| | LC-MS (Method 1): Rt 1.34 = min; MS (ESIpos): m/z = 608 [M + H]⁺ |
| Intermediate 556 | 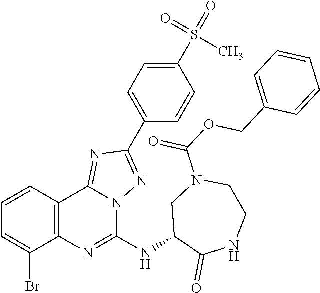 [SMILES: CS(=O)(=O)c1ccc(-c2nc3c4cccc(Br)c4nc(N[C@@H]4CN(C(=O)OCc5ccccc5)CCNC4=O)n3n2)cc1] |
| | benzyl (6R)-6-({7-bromo-2-[4- |
| | (methanesulfonyl)phenyl][1,2,4]triazolo[1,5-c]quinazolin-5-yl}amino)-5- |
| | oxo-1,4-diazepane-1-carboxylate |
| | LC-MS (method 2): Rₜ = 1.30 min; MS (ESIpos): m/z = 664 [M + H]⁺ |
| Intermediate 557 | 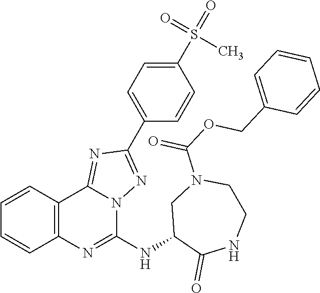 [SMILES: CS(=O)(=O)c1ccc(-c2nc3c4ccccc4nc(N[C@@H]4CN(C(=O)OCc5ccccc5)CCNC4=O)n3n2)cc1] |
| | benzyl (6R)-6-({2-[4-(methanesulfonyl)phenyl][1,2,4]triazolo[1,5- |
| | c]quinazolin-5-yl}amino)-5-oxo-1,4-diazepane-1-carboxylate |
| | LC-MS (method 2): Rₜ = 1.24 min; MS (ESIpos): m/z = 586 [M + H]⁺ |

### Intermediate 558

### N-[(E)-(dimethylamino)methylidene]-2-nitrobenzamide

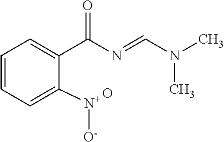

2-Nitrobenzamide (1.00 g, 6.02 mmol) and 1,1-dimethoxy-N,N-dimethylmethanamine (4.0 mL, 30 mmol) were stirred at 100° C. for 1 h. The mixture was filtered, washed with hexane and dried under reduced pressure at 50° C. to give 1.03 g (100% purity, 78% yield) of the target compound, which was used without further purification.

LC-MS (Method 2): Rₜ=0.76 min; MS (ESIpos): m/z=222 [M+H]⁺

¹H-NMR (400 MHz, DMSO-d6): δ [ppm]=3.03 (d, 3H), 3.20 (s, 3H), 7.63-7.73 (m, 2H), 7.78-7.82 (m, 1H), 7.92 (dd, 1H), 8.58 (t, 1H).

### Intermediate 559

### 5-(2-nitrophenyl)-1H-1,2,4-triazole

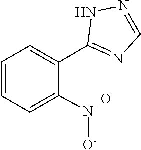

N-[(E)-(Dimethylamino)methylidene]-2-nitrobenzamide (1.03 g, 4.67 mmol), hydrazine-water (1/1) (570 μL, 12 mmol) and molecular sieves (3 A) were stirred in acetic acid (26 mL, 450 mmol) and butan-1-ol (15 mL) at reflux for 2 h. The reaction mixture was diluted with water and extracted with EtOAc. The organic layer was washed with sat. sodium hydrogen carbonate solution, dried and concentrated under reduced pressure to give 885 mg (100% purity, 100% yield) of the target compound, which was used without further purification.

LC-MS (Method 4): Rₜ=0.87 min; MS (ESIneg): m/z=189 [M−H]⁻.

¹H-NMR (400 MHz, DMSO-d6): δ [ppm]=7.65 (td, 1H), 7.76 (td, 1H), 7.88 (dd, 1H), 7.99 (dd, 1H), 8.62 (s, 1H).

### Intermediate 560

### 3-bromo-5-(2-nitrophenyl)-4H-1,2,4-triazole

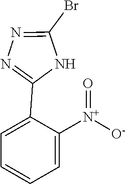 [SMILES: O=[N+]([O-])c1ccccc1-c1nnc(Br)[nH]1]

3-(2-Nitrophenyl)-4H-1,2,4-triazole (8.66 g, 45.5 mmol), NBS (17.8 g, 100 mmol) and potassium carbonate (31.5 g, 228 mmol) were stirred in DMF (220 mL) for 2 h at rt. The mixture was evaporated, diluted with ethanol, filtered and the filtrate was concentrated under reduced pressure to give 33.0 g (37% purity) of the target compound, which was used without further purification.

1H-NMR (400 MHz, DMSO-d6): δ [ppm]=7.38 (td, 1H), 7.53-7.61 (m, 2H), 7.92-7.95 (m, 2H).

### Intermediate 561

### 2-(5-bromo-4H-1,2,4-triazol-3-yl)aniline

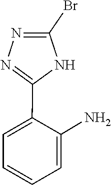 [SMILES: Nc1ccccc1-c1nnc(Br)[nH]1]

3-Bromo-5-(2-nitrophenyl)-4H-1,2,4-triazole (21.5 g, 79.9 mmol) was solubilised in methanol (520 mL) and THF (160 mL), platinium/vanadium (7.79 g, 1% purity, 400 μmol) was added and the mixture was stirred under an hydrogen atmosphere at rt for 6 h. The mixture was filtered and washed with methanol and THF. The filtrate was concentrated under reduced pressure to give 16.9 g (90% purity, 80% yield) of the target compound, which was used without further purification.

LC-MS (Method 4): Rₜ=1.23 min; MS (ESIpos): m/z=239 [M+H]⁺

¹H-NMR (400 MHz, DMSO-d₆): δ [ppm]=6.47 (ddd, 1H), 6.63 (dd, 1H), 6.90 (ddd, 1H), 7.76 (dd, 1H), 11.07 (br s, 2H).

### Intermediate 562

### 2-bromo[1,2,4]triazolo[1,5-c]quinazolin-5(6H)-one

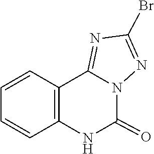 [SMILES: O=c1[nH]c2ccccc2c2nc(Br)nn12]

2-(5-Bromo-4H-1,2,4-triazol-3-yl)aniline (15.2 g, 63.7 mmol) and di(1H-imidazol-1-yl)methanone (24.7 g, 152.9 mmol) were stirred in DMF (250 mL) at rt overnight. The mixture was evaporated, diluted with ACN/water (95:5), filtered, washed with ACN and dried under reduced pressure at 60° C. to give 1.99 g (95% purity, 11% yield) of the target compound, which was used without further purification.

LC-MS (Method 4): Rₜ=1.20 min; MS (ESIpos): m/z=265 [M+H]⁺

¹H-NMR (400 MHz, DMSO-d₆): δ [ppm]=6.97 (ddd, 1H), 7.18 (d, 1H), 7.39 (ddd, 1H), 7.87 (dd, 1H).

### Intermediate 563

### 2-bromo-5-chloro[1,2,4]triazolo[1,5-c]quinazoline

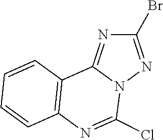 [SMILES: Clc1nc2ccccc2c2nc(Br)nn12]

2-Bromo[1,2,4]triazolo[1,5-c]quinazolin-5(6H)-one (4.41 g, 16.6 mmol) was stirred in phosphorus oxychloride (54 mL, 580 mmol) and N,N-diisopropylethylamine (29 mL, 170 mmol) for 4 h at 110° C. The mixture was poured into ice, filtered, washed with water and dried at 60° C. under reduced pressure to give 3.00 g (64% yield) of the target compound, which was used without further purification.

LC-MS (Method 2): Rₜ=1.13 min; MS (ESIpos): m/z=283 [M+H]⁺

¹H-NMR (400 MHz, DMSO-d₆): δ [ppm]=7.87 (ddd, 1H), 7.97-8.09 (m, 2H), 8.40-8.47 (m, 1H).

### Intermediate 564

### (3R)-3-[(2-bromo[1,2,4]triazolo[1,5-c]quinazolin-5-yl)amino]azepan-2-one

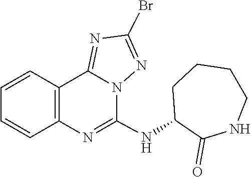 [SMILES: O=C1NCCCC[C@H]1Nc1nc2ccccc2c2nc(Br)nn12]

2-Bromo-5-chloro[1,2,4]triazolo[1,5-c]quinazoline (3.00 g, 10.6 mmol), (3R)-3-aminoazepan-2-one hydrochloride (1.92 g, 11.6 mmol) and N,N-diisopropylethylamine (7.4 mL, 42 mmol) were stirred in DMSO (80 mL) for 2 h at 60° C. The mixture was diluted with water, filtered, washed with water and dried under reduced pressure at 60° C. to give 3.72 g (95% purity, 89% yield) of the target compound, which was used without further purification.

LC-MS (Method 2): Rₜ=1.18 min; MS (ESIpos): m/z=375 [M+H]⁺

¹H-NMR (400 MHz, DMSO-d₆): δ [ppm]=1.24-1.37 (m, 1H), 1.47-1.60 (m, 1H), 1.78-1.91 (m, 2H), 1.96-2.05 (m, 1H), 2.27 (br d, 1H), 3.10-3.20 (m, 1H), 4.78 (dd, 1H), 7.45 (ddd, 1H), 7.63-7.70 (m, 2H), 7.73-7.79 (m, 1H), 8.16-8.23 (m, 2H).

### Intermediate 565

### tert-butyl 3-[4-(5-{[(3R)-2-oxoazepan-3-yl]amino}[1,2,4]triazolo[1,5-c]quinazolin-2-yl)-1H-pyrazol-1-yl]azetidine-1-carboxylate

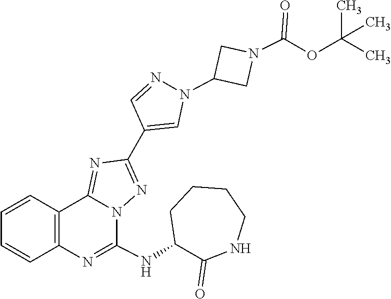 [SMILES: CC(C)(C)OC(=O)N1CC(n2cc(-c3nc4c5ccccc5nc(N[C@@H]5CCCCNC5=O)n4n3)cn2)C1]

(3R)-3-[(2-Bromo[1,2,4]triazolo[1,5-c]quinazolin-5-yl)amino]azepan-2-one (75.0 mg, 200 μmol), tert-butyl 3-[4-(4,4,5,5-tetramethyl-1,3,2-dioxaborolan-2-yl)-1H-pyrazol-1-yl]azetidine-1-carboxylate (90.7 mg, 260 μmol) and XPhos Pd G1 (8.26 mg, 9.99 μmol) were solubilised in DMF (1.3 mL) and aqueous K₂CO₃ (300 μl, 2.0 M, 600 μmol). The mixture was sparged with argon and stirred for 1 h at 110° C. The mixture was cooled to rt and purified by preparative HPLC to give 46.1 mg (100% purity, 45% yield) of the title compound.

LC-MS (method 2): Rₜ=1.24 min; MS (ESIpos): m/z=518 [M+H]⁺

¹H-NMR (400 MHz, DMSO-d6): δ [ppm]=1.25-1.38 (m, 1H), 1.42 (s, 9H), 1.48-1.60 (m, 1H), 1.80-1.95 (m, 2H), 1.97-2.06 (m, 1H), 2.26-2.34 (m, 1H), 3.10-3.21 (m, 1H), 4.22 (br s, 2H), 4.29-4.37 (m, 2H), 4.82 (br dd, 1H), 5.35 (tt, 1H), 7.44 (ddd, 1H), 7.61 (br d, 1H), 7.63-7.68 (m, 1H), 7.69-7.75 (m, 1H), 8.18-8.26 (m, 3H), 8.66 (s, 1H).

### Intermediate 566

### tert-butyl 4-[4-(5-{[(3R)-2-oxoazepan-3-yl]amino}[1,2,4]triazolo[1,5-c]quinazolin-2-yl)-1H-pyrazol-1-yl]piperidine-1-carboxylate

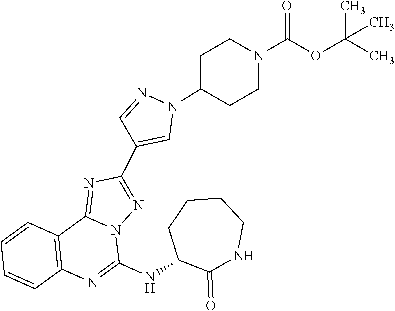 [SMILES: CC(C)(C)OC(=O)N1CCC(n2cc(-c3nc4c5ccccc5nc(N[C@@H]5CCCCNC5=O)n4n3)cn2)CC1]

(3R)-3-{[2-(1H-pyrazol-4-yl)[1,2,4]triazolo[1,5-c]quinazolin-5-yl]amino}azepan-2-one (75.0 mg, 207 μmol), tert-butyl 4-bromopiperidine-1-carboxylate (60.1 mg, 228 μmol) and Cs₂CO₃ (202 mg, 621 μmol) were stirred in DMF (440 μl) at 100° C. for 1 h. The reaction mixture was cooled to rt and purified by preparative HPLC to give 38.8 mg (95% purity, 33% yield) of the title compound.

LC-MS (method 2): Rₜ=1.33 min; MS (ESIpos): m/z=546 [M+H]⁺

¹H-NMR (400 MHz, DMSO-d6): δ [ppm]=1.25-1.38 (m, 1H), 1.43 (s, 9H), 1.48-1.59 (m, 1H), 1.80-1.95 (m, 4H), 1.97-2.12 (m, 3H), 2.26-2.35 (m, 1H), 2.82-3.03 (m, 2H), 3.10-3.21 (m, 1H), 4.07 (br d, 2H), 4.49 (tt, 1H), 4.82 (br dd, 1H), 7.43 (ddd, 1H), 7.59 (d, 1H), 7.62-7.67 (m, 1H), 7.69-7.74 (m, 1H), 8.10 (s, 1H), 8.19-8.26 (m, 2H), 8.56 (s, 1H).

### Intermediate 567

### benzyl (6R)-6-[(2-bromo[1,2,4]triazolo[1,5-c]quinazolin-5-yl)amino]-5-oxo-1,4-diazepane-1-carboxylate

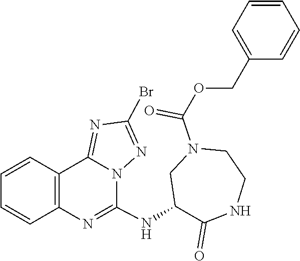 [SMILES: O=C1NCCN(C(=O)OCc2ccccc2)C[C@H]1Nc1nc2ccccc2c2nc(Br)nn12]

2-Bromo-5-chloro[1,2,4]triazolo[1,5-c]quinazoline (1.00 g, 3.53 mmol) and benzyl (6R)-6-amino-5-oxo-1,4-diazepane-1-carboxylate hydrochloride (1.16 g, 3.88 mmol) were suspended in DMSO (10 mL), treated with N,N-diisopropylethylamine (2.5 mL, 14 mmol) and the mixture was stirred for 2 h at 60° C. The mixture was poured into water, the precipitate was filtered, washed with water and dried under reduced pressure to give 1.78 g (100% purity, 99% yield) of the target compound.

LC-MS (Method 2): Rₜ=1.24 min; MS (ESIpos): m/z=510 [M+H]⁺

### Intermediate 568

### tert-butyl 3-[2-(4-methoxyphenyl)-5-{[(3R)-2-oxoazepan-3-yl]amino}[1,2,4]triazolo[1,5-c]quinazolin-10-yl]azetidine-1-carboxylate

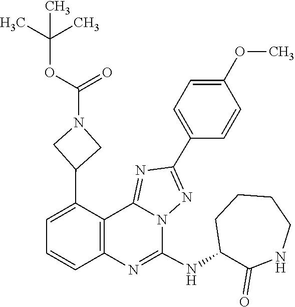 [SMILES: COc1ccc(-c2nc3c4c(C5CN(C(=O)OC(C)(C)C)C5)cccc4nc(N[C@@H]4CCCCNC4=O)n3n2)cc1]

(3R)-3-{[10-Bromo-2-(4-methoxyphenyl)[1,2,4]triazolo[1,5-c]quinazolin-5-yl]amino}azepan-2-one (50.0 mg, 104 μmol), lithium carbonate (46.1 mg, 623 μmol) and (4,4′-di-tert-butyl-2,2′-bipyridine-kappa²N¹,N¹)(bis{3,5-difluoro-2-[5-(trifluoromethyl)pyridin-2-yl-kappaN]phenyl-kappaC¹})iridium(1+) hexafluorophosphate (2.33 mg, 2.08 μmol) were dissolved in a reaction vial in (trifluoromethyl)benzene (2.0 mL). In a separate vial, 1,2-dimethoxyethane-dichloronickel (1:1) (110 μg, 0.52 μmol) and 4,4′-di-tert-butyl-2,2′-bipyridine (140 μg, 0.52 μmol) were stirred in N,N-dimethylacetamide (1000 μL, 11 mmol) for 5 min. The catalyst solution was added to the sealed reaction vial. The mixture was degassed under vacuum in an ultrasound bath for 5 min and purging with argon after. Then tert-butyl 3-bromoazetidine-1-carboxylate (76 μL, 470 μmol) and 1,1,1,3,3,3-hexamethyl-2-(trimethylsilyl)trisilane (32 μL, 100 μmol) were added. The vial was stirred in a water bath and irradiated by two 40 W Kessil LED Aquarium lamps (A160WE tuna blue). The mixture was evaporated, diluted with DMSO and filtered. The solid was diluted with water and extracted three times with DCM. The combined organic layers were dried and concentrated under reduced pressure. The residue was purified by preparative HPLC to give 9.10 mg (100% purity, 16% yield) of the target compound.

LC-MS (Method 2): Rₜ=1.59 min; MS (ESIpos): m/z=558 [M+H]⁺

¹H-NMR (400 MHz, DMSO-d₆): δ [ppm]=1.25-1.37 (m, 1H), 1.40 (s, 9H), 1.49-1.62 (m, 1H), 1.80-1.94 (m, 2H), 1.97-2.07 (m, 1H), 2.27-2.35 (m, 1H), 3.10-3.22 (m, 1H), 3.86 (s, 3H), 3.94-4.05 (m, 2H), 4.51 (br t, 2H), 4.80 (br dd, 1H), 4.99-5.10 (m, 1H), 7.12-7.18 (m, 2H), 7.54 (dd, 2H), 7.65-7.73 (m, 2H), 8.18-8.25 (m, 3H).

### Intermediate 569

### (3R)-3-{[2-(4-methoxyphenyl)-7-(prop-1-en-2-yl)[1,2,4]triazolo[1,5-c]quinazolin-5-yl]amino}azepan-2-one

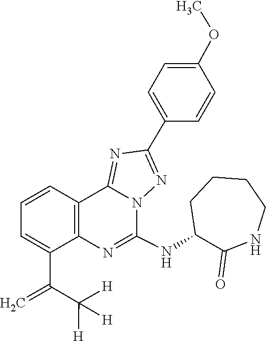 [SMILES: C=C(C)c1cccc2c1nc(N[C@@H]1CCCCNC1=O)n1nc(-c3ccc(OC)cc3)nc21]

(3R)-3-{[7-Bromo-2-(4-methoxyphenyl)[1,2,4]triazolo[1,5-c]quinazolin-5-yl]amino}azepan-2-one (100 mg, 208 μmol) and XPhos Pd G4 (89.4 mg, 104 μmol) were dissolved in 1,4-dioxane (5.0 mL), aqueous sodium carbonate solution (270 μL, 2.0 M, 540 μmol) was added and the mixture was sparged with argon for 5 min. 4,4,5,5-Tetramethyl-2-(prop-1-en-2-yl)-1,3,2-dioxaborolane (69.8 mg, 415 μmol) was added and the mixture was stirred for 2 h at 100° C. Water was added to the mixture and extracted two times with EtOAc. The combined organic layers were dried and the solvent was evaporated. The residue was purified by preparative HPLC to give 34.2 mg (95% purity, 35% yield) of the target compound.

LC-MS (Method 2): Rₜ=1.49 min; MS (ESIpos): m/z=443 [M+H]⁺

1H-NMR (400 MHz, DMSO-d6): δ [ppm]=1.21-1.40 (m, 2H), 1.49-1.64 (m, 1H), 1.80-1.96 (m, 2H), 1.97-2.14 (m, 2H), 2.36 (s, 3H), 3.19 (br s, 1H), 3.86 (s, 3H), 4.70-4.88 (m, 1H), 5.20 (s, 1H), 5.30 (s, 1H), 7.14 (d, 2H), 7.36-7.44 (m, 1H), 7.57-7.76 (m, 1H), 7.58-7.94 (m, 1H), 8.14-8.27 (m, 4H).

### Intermediate 570

### benzyl (6R)-6-{[2-(4-methoxyphenyl)-7-(3,3,3-trifluoroprop-1-en-2-yl)[1,2,4]triazolo[1,5-c]quinazolin-5-yl]amino}-5-oxo-1,4-diazepane-1-carboxylate

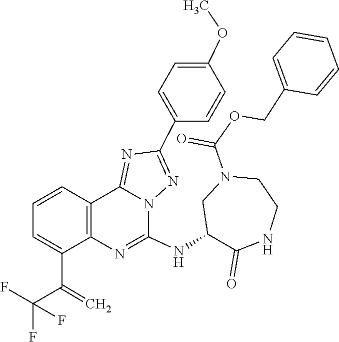 [SMILES: C=C(c1cccc2c1nc(N[C@@H]1CN(C(=O)OCc3ccccc3)CCNC1=O)n1nc(-c3ccc(OC)cc3)nc21)C(F)(F)F]

Benzyl (6R)-6-{[7-bromo-2-(4-methoxyphenyl)[1,2,4]triazolo[1,5-c]quinazolin-5-yl]amino}-5-oxo-1,4-diazepane-1-carboxylate (194 mg, 315 μmol), 4,4,5,5-tetramethyl-2-(3,3,3-trifluoroprop-1-en-2-yl)-1,3,2-dioxaborolane (140 μL, 630 μmol) and XPhosPdG4 (13.5 mg, 15.7 μmol) were solubilised in 1,4-dioxane (6.0 mL). Aqueous Na₂CO₃ (410 μL, 2.0 M, 820 μmol) was added and the mixture was sparged with argon (while sonicating). The reaction mixture was stirred at 100° C. for 5 h. XPhosPdG4 (13.5 mg, 15.7 μmol), aqueous Na₂CO₃ (410 μL, 2.0 M, 820 μmol) and 4,4,5,5-tetramethyl-2-(3,3,3-trifluoroprop-1-en-2-yl)-1,3,2-dioxaborolane (140 μL, 630 μmol) we added again and the reaction was stirred for another 5 h at 100° C. The reaction mixture was cooled to rt and diluted with water and extracted with EtOAc. The organic phase was dried (silicon filter) and concentrated under reduced pressure. The crude material was purified by flash chromatography to give 174 mg (95% purity, 83% yield) of the title compound.

LC-MS (method 2): Rₜ=1.46 min; MS (ESIneg): m/z=630 [M−H]⁻

¹H-NMR (400 MHz, DMSO-d6) δ[ppm]: 1.066 (0.83), 1.172 (0.44), 1.231 (0.49), 1.988 (0.53), 2.518 (3.05), 2.523 (2.09), 3.092 (0.43), 3.865 (16.00), 5.145 (0.60), 5.759 (2.13), 7.142 (0.57), 7.149 (3.98), 7.154 (1.28), 7.166 (1.34), 7.172 (4.15), 7.179 (0.65), 7.388 (0.77), 7.478 (1.33), 7.497 (2.05), 7.516 (1.57), 7.668 (1.04), 7.684 (0.84), 8.244 (3.23), 8.249 (1.34), 8.261 (1.50), 8.266 (3.31), 8.377 (0.97), 8.380 (0.99), 8.397 (0.95), 8.400 (0.92).

### Intermediate 571

### benzyl (6R)-6-({2-(4-methoxyphenyl)-7-[1-(trifluoromethyl)cyclopropyl][1,2,4]triazolo[1,5-c]quinazolin-5-yl}amino)-5-oxo-1,4-diazepane-1-carboxylate

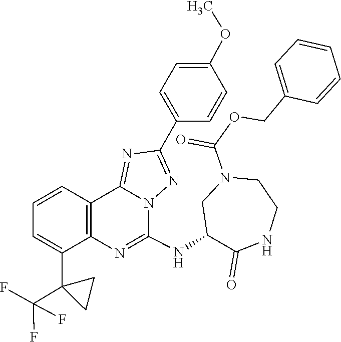 [SMILES: COc1ccc(-c2nc3c4cccc(C5(C(F)(F)F)CC5)c4nc(N[C@@H]4CN(C(=O)OCc5ccccc5)CCNC4=O)n3n2)cc1]

Benzyl (6R)-6-{[2-(4-methoxyphenyl)-7-(3,3,3-trifluoroprop-1-en-2-yl)[1,2,4]triazolo[1,5-c]quinazolin-5-yl]amino}-5-oxo-1,4-diazepane-1-carboxylate (164 mg, 260 μmol), triethylammonium bis(catecholato)iodomethylsilicate (253 mg, 519 μmol) and 2,4,5,6-Tetra(9H-carbazol-9-yl)isophthalonitrile (10.2 mg, 13.0 μmol) were solubilised in DMSO (5.2 mL) and the reaction was degassed with argon for 5 min. The reaction was placed in a water bath (to keep the temp. below 35° C.) and was subsequently irradiated by two 40 W Kessil LED Aquarium lamps for 12 h. Aqueous 1M NaOH was added and the mixture was extracted for three times with EtOAc. The organic phase was washed with aqueous 1M NaOH followed by saturated aq. NaCl. The organic phase was dried (silicon filter) and concentrated under reduced pressure. The crude material was purified by flash chromatography to give 90 mg (54% yield) of the title compound.

LC-MS (method 2): Rₜ=1.46 min; MS (ESIpos): m/z=646 [M+H]⁺

### Intermediate 572

### benzyl (6R)-6-{[2-(1-methyl-1H-pyrazol-4-yl)-7-(3,3,3-trifluoroprop-1-en-2-yl)[1,2,4]triazolo[1,5-c]quinazolin-5-yl]amino}-5-oxo-1,4-diazepane-1-carboxylate

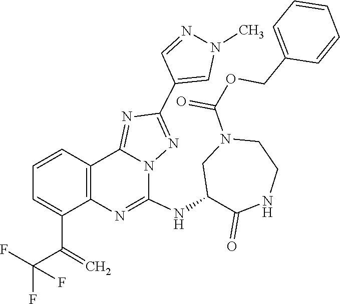 [SMILES: C=C(c1cccc2c1nc(N[C@@H]1CN(C(=O)OCc3ccccc3)CCNC1=O)n1nc(-c3cnn(C)c3)nc21)C(F)(F)F]

Benzyl (6R)-6-{[7-bromo-2-(1-methyl-1H-pyrazol-4-yl)[1,2,4]triazolo[1,5-c]quinazolin-5-yl]amino}-5-oxo-1,4-diazepane-1-carboxylate (160 mg, 271 μmol), 4,4,5,5-tetramethyl-2-(3,3,3-trifluoroprop-1-en-2-yl)-1,3,2-dioxaborolane (120 μL, 540 μmol) and XPhosPdG4 (11.7 mg, 13.5 μmol) were solubilised in the solvent 1,4-dioxane (5.2 mL). Aqueous Na₂CO₃ (350 μl, 2.0 M, 700 μmol) was added and the mixture was sparged with argon (while sonicating). The reaction mixture was stirred at 100° C. for 5 h. The reaction mixture was cooled to rt and diluted with water and extracted with EtOAc. The organic phase was dried (silicon filter) and concentrated under reduced pressure. The crude material was purified by flash chromatography to give 151 mg (95% purity, 87% yield) of the title compound.

LC-MS (method 2): Rₜ=1.23 min; MS (ESIpos): m/z=606 [M+H]⁺

¹H-NMR (400 MHz, DMSO-d6) δ[ppm]: 1.066 (1.07), 1.154 (3.19), 1.172 (6.71), 1.190 (3.39), 1.231 (0.74), 1.988 (10.78), 2.337 (0.42), 2.518 (5.25), 2.523 (3.52), 2.678 (0.42), 3.085 (0.57), 3.119 (0.42), 3.964 (16.00), 3.999 (0.87), 4.017 (2.56), 4.035 (2.54), 4.053 (0.96), 5.140 (0.87), 5.759 (1.49), 7.050 (0.42), 7.191 (0.48), 7.384 (1.07), 7.466 (2.03), 7.485 (2.93), 7.505 (2.16), 7.659 (1.38), 7.676 (1.14), 8.079 (4.52), 8.088 (0.57), 8.279 (0.55), 8.322 (1.46), 8.325 (1.46), 8.342 (1.36), 8.479 (3.43).

### Intermediate 573

### benzyl (6R)-6-({2-(1-methyl-1H-pyrazol-4-yl)-7-[1-(trifluoromethyl)cyclopropyl][1,2,4]triazolo[1,5-c]quinazolin-5-yl}amino)-5-oxo-1,4-diazepane-1-carboxylate

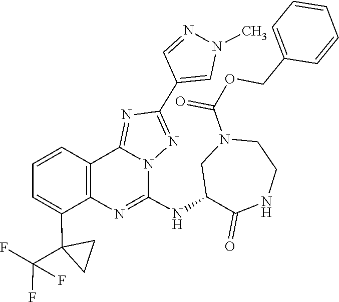 [SMILES: Cn1cc(-c2nc3c4cccc(C5(C(F)(F)F)CC5)c4nc(N[C@@H]4CN(C(=O)OCc5ccccc5)CCNC4=O)n3n2)cn1]

benzyl (6R)-6-{[2-(1-methyl-1H-pyrazol-4-yl)-7-(3,3,3-trifluoroprop-1-en-2-yl)[1,2,4]triazolo[1,5-c]quinazolin-5-yl]amino}-5-oxo-1,4-diazepane-1-carboxylate (141 mg, 233 μmol), triethylammonium bis(catecholato)iodomethylsilicate (227 mg, 466 μmol) and 2,4,5,6-Tetra(9H-carbazol-9-yl)isophthalonitrile (9.18 mg, 11.6 μmol) were solubilised in DMSO (4.7 mL) and the reaction mixture was degassed with argon for 5 min. The reaction was placed in a water bath (to keep the temp. below 35° C.) and was subsequently irradiated by two 40 W Kessil LED Aquarium lamps for 12 h. Aqueous 1M NaOH was added and the mixture was extracted for three times with EtOAc. The organic phase was washed with aqueous 1M NaOH followed by saturated aq. NaCl. The organic phase was dried (silicon filter) and concentrated under reduced pressure. The crude material was purified by flash chromatography to give 67.0 mg (46% yield) of the title compound.

LC-MS (method 2): Rₜ=1.25 min; MS (ESIpos): m/z=620 [M+H]⁺

¹H-NMR (400 MHz, DMSO-d6) δ[ppm]: 1.154 (2.72), 1.172 (3.85), 1.190 (1.92), 1.231 (0.76), 1.433 (1.13), 1.988 (5.26), 2.518 (3.92), 2.523 (2.73), 2.674 (0.74), 3.161 (0.50), 3.178 (0.62), 3.196 (0.55), 3.212 (0.43), 3.962 (16.00), 3.999 (0.65), 4.017 (1.46), 4.035 (1.50), 4.053 (0.69), 5.012 (0.45), 5.130 (0.58), 7.085 (0.43), 7.206 (0.46), 7.366 (0.86), 7.430 (1.89), 7.450 (2.82), 7.469 (2.03), 7.820 (1.53), 7.824 (1.58), 7.838 (1.41), 7.842 (1.34), 8.072 (4.61), 8.074 (4.78), 8.240 (0.84), 8.254 (1.27), 8.268 (0.84), 8.278 (2.37), 8.282 (2.25), 8.297 (2.10), 8.301 (1.92), 8.464 (4.30).

### Intermediate 574

### tert-butyl [(2R)-1-(4-methylpiperazin-1-yl)-1-oxopropan-2-yl]carbamate

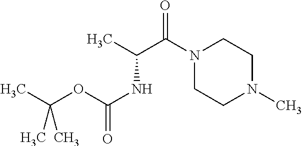 [SMILES: C[C@@H](NC(=O)OC(C)(C)C)C(=O)N1CCN(C)CC1]

N-(tert-Butoxycarbonyl)-D-alanine (1.00 g, 5.29 mmol) was dissolved in THF (11 mL). 4-Methylmorpholine (580 μL, 5.3 mmol) and 2-methylpropyl carbonochloridate (690 μL, 5.3 mmol) were added at −20° C. It was stirred for 5 min at −20° C. 1-Methylpiperazine (700 μL, 6.3 mmol) in THF (2 mL) was added dropwise at −20° C. It was stirred for 2 h at −20° C. The reaction mixture was allowed to reach rt and aqueous sodium hydrogen carbonate solution (5%) was added. The reaction mixture was extracted with dichloromethane (three times 20 mL). The combined organic phases were dried over magnesium sulfate, filtered and concentrated affording 1.63 g of the title product which was used without further purification in the next step.

¹H-NMR (400 MHz, DMSO-d₆): δ [ppm]=1.11 (d, 3H), 1.36 (s, 9H), 2.17 (s, 3H), 2.20-2.31 (m, 4H), 3.39-3.48 (m, 4H), 4.36-4.45 (m, 1H), 6.93 (d, 1H).

The following intermediates were prepared analogously to intermediate 574.

| | Structure |
|---|---|
| | IUPAC-Name |
| | LC-MS (method): Retention time; Mass found |
| Intermediate | ¹H-NMR |
| Intermediate 575 | 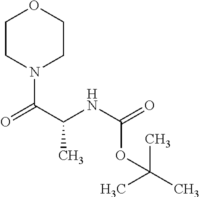 [SMILES: C[C@@H](NC(=O)OC(C)(C)C)C(=O)N1CCOCC1] |
| | tert-butyl [(2R)-1-(morpholin-4-yl)-1-oxopropan-2-yl]carbamate |
| | ¹H-NMR (400 MHz, DMSO-d₆): δ [ppm] = 1.12 (d, 3H), 1.37 (s, 9H), 3.40- |
| | 3.51 (m, 4H), 3.51-3.59 (m, 4H), 4.35-4.46 (m, 1H), 6.99 (d, 1H). |
| Intermediate 576 | 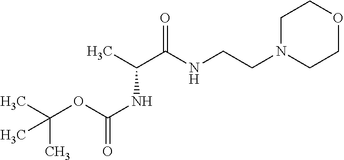 [SMILES: C[C@@H](NC(=O)OC(C)(C)C)C(=O)NCCN1CCOCC1] |
| | tert-butyl [(2R)-1-{[2-(morpholin-4-yl)ethyl]amino}-1-oxopropan-2- |
| | yl]carbamate |
| | ¹H-NMR (400 MHz, DMSO-d₆): δ [ppm] = 1.14 (d, 3H), 1.37 (s, 9H), 2.28- |
| | 2.39 (m, 6H), 3.04-3.25 (m, 2H), 3.55 (t, 4H), 3.86-3.95 (m, 1H), 6.90 (br |
| | d, 1H), 7.64 (br t, 1H). |
| Intermediate 577 | 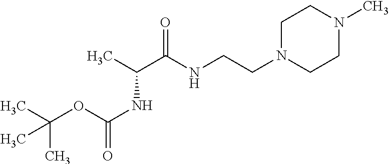 [SMILES: C[C@@H](NC(=O)OC(C)(C)C)C(=O)NCCN1CCN(C)CC1] |
| | tert-butyl [(2R)-1-{[2-(4-methylpiperazin-1-yl)ethyl]amino}-1- |
| | oxopropan-2-yl]carbamate |
| | ¹H-NMR (400 MHz, DMSO-d₆): δ [ppm] = 1.14 (d, 3H), 1.38 (s, 9H), 2.13 (s, |
| | 3H), 2.20-2.43 (m, 10H), 3.02-3.23 (m, 2H), 3.90 (quin, 1H), 6.85-6.96 |
| | (m, 1H), 7.59 (br t, 1H). |
| Intermediate 578 | 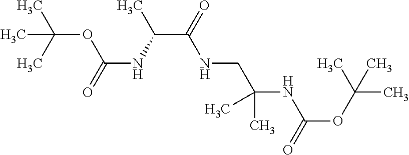 [SMILES: C[C@@H](NC(=O)OC(C)(C)C)C(=O)NCC(C)(C)NC(=O)OC(C)(C)C] |
| | N²-(tert-butoxycarbonyl)-N-{2-[(tert-butoxycarbonyl)amino]-2- |
| | methylpropyl}-D-alaninamide |
| | ¹H-NMR (400 MHz, DMSO-d₆): δ [ppm] = 1.12 (s, 6H), 1.17 (s, 3H), 1.37 (s, |
| | 9H), 1.37 (s, 9H), 3.09 (dd, 1H), 3.23 (dd, 1H), 3.92 (quin, 1H), 6.47 (br s, |
| | 1H), 6.98 (br d, 1H), 7.71 (b, t, 1H). |
| Intermediate 579 | 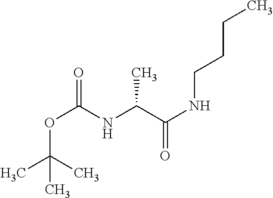 [SMILES: CCCCNC(=O)[C@@H](C)NC(=O)OC(C)(C)C] |
| | tert-butyl [(2R)-1-(butylamino)-1-oxopropan-2-yl]carbamate |
| | ¹H-NMR (400 MHz, DMSO-d₆): δ [ppm] = 0.85 (t, 3H), 1.14 (d, 3H), 1.20- |
| | 1.41 (m, 13H), 2.91-3.11 (m, 2H), 3.84-3.94 (m, 1H), 6.81 (br d, 1H), |
| | 7.68 (br t, 1H). |
| Intermediate 580 | 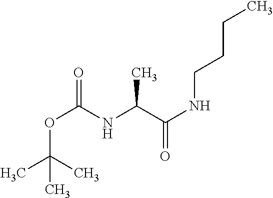 [SMILES: CCCCNC(=O)[C@H](C)NC(=O)OC(C)(C)C] |
| | tert-butyl [(2S)-1-(butylamino)-1-oxopropan-2-yl]carbamate |
| | ¹H-NMR (400 MHz, DMSO-d₆): δ [ppm] = 0.85 (t, 3H), 1.14 (d, 3H), 1.20- |
| | 1.31 (m, 2H), 1.32-1.41 (m, 11H), 2.91-3.11 (m, 2H), 3.89 (quin, 1H), |
| | 6.81 (br d, 1H), 7.68 (br t, 1H). |
| Intermediate 581 | 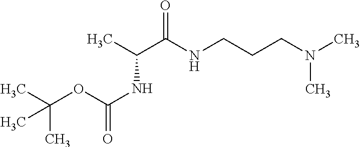 [SMILES: C[C@@H](NC(=O)OC(C)(C)C)C(=O)NCCCN(C)C] |
| | N²-(tert-butoxycarbonyl)-N-[3-(dimethylamino)propyl]-D-alaninamide |
| | ¹H-NMR (400 MHz, DMSO-d₆): δ [ppm] = 1.14 (d, 3H), 1.37 (s, 9H), 1.50 (br |
| | quin, 2H), 2.10 (s, 6H), 2.18 (t, 2H), 2.97-3.12 (m, 2H), 3.88 (quin, 1H), |
| | 6.84 (d, 1H), 7.76 (br t, 1H). |
| Intermediate 582 | 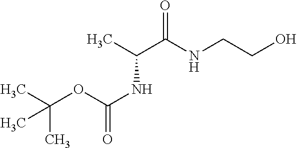 [SMILES: C[C@@H](NC(=O)OC(C)(C)C)C(=O)NCCO] |
| | N²-(tert-butoxycarbonyl)-N-(2-hydroxyethyl)-D-alaninamide |
| | ¹H-NMR (400 MHz, DMSO-d₆): δ [ppm] = 1.14 (d, 3H), 1.37 (s, 9H), 3.05- |
| | 3.16 (m, 2H), 3.37 (q, 2H), 3.92 (quin, 1H), 4.65 (t, 1H), 6.86 (br d, 1H), |
| | 7.71 (br t, 1H). |

### Intermediate 583

### (2R)-2-amino-1-(4-methylpiperazin-1-yl)propan-1-one-hydrogen chloride (1/2)

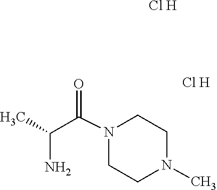

tert-butyl [(2R)-1-(4-Methyl piperazin-1-yl)-1-oxopropan-2-yl]carbamate (1.63 g, 6.01 mmol) was dissolved in 1,4-dioxane (12.8 mL). Hydrochloride in 1,4-dioxane (7.5 mL, 4M) was added and stirred overnight at rt. It was concentrated under reduced pressure on a rotary evaporator. MTBE was added to the residue and stirred for some minutes. The solid material was filtered, washed twice with MTBE and dried under reduced pressure affording 1.125 (77%) of the title compound which was used without further purification in the next step.

¹H-NMR (400 MHz, DMSO-d₆): δ [ppm]=1.33 (br s, 3H), 2.75 (s, 3H), 2.82-3.28 (m, 4H), 3.50-3.68 (m, 1H), 3.98-4.19 (m, 1H), 4.28-4.52 (m, 2H), 8.34 (br d, 3H).

The following intermediates were prepared analogously to intermediate 583.

| | Structure |
|---|---|
| | IUPAC-Name |
| | LC-MS (method): Retention time; Mass found |
| Intermediate | ¹H-NMR |
| Intermediate 584 | 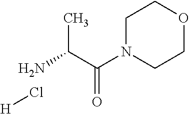 |
| | (2R)-2-amino-1-(morpholin-4-yl)propan-1-one-hydrogen chloride (1/1) |
| | ¹H-NMR (400 MHz, DMSO-d₆): δ [ppm] = 1.30 (d, 3H), 3.38-3.66 (m, 8H), 4.35 |
| | (q, 1H), 8.19 (br s, 3H). |
| Intermediate 585 | 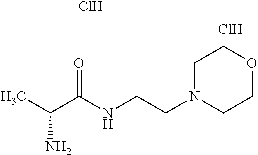 |
| | N-[2-(morpholin-4-yl)ethyl]-D-alaninamide-hydrogen chloride (1/2) |
| | ¹H-NMR (400 MHz, DMSO-d₆): δ [ppm] = 1.36 (d, 3H), 3.00-3.27 (m, 4H), 3.39- |
| | 3.56 (m, 3H), 3.59-3.70 (m, 1H), 3.81-3.99 (m, 5H), 8.31 (br s, 3H), 8.94 |
| | (br s, 1H), 11.04 (br s, 1H). |
| Intermediate 586 | 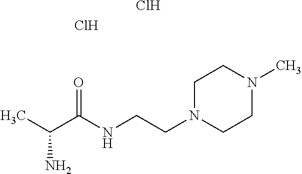 |
| | N-[2-(4-methylpiperazin-1-yl)ethyl]-D-alaninamide-hydrogen chloride |
| | (1/2) |
| | ¹H-NMR (400 MHz, DMSO-d₆): δ [ppm] = 1.37 (d, 3H), 2.81 (br s, 3H), 2.96- |
| | 3.29 (m, 3H), 3.56-3.91 (m, 8H), 8.29 (br s, 3H), 8.75-8.94 (m, 1H), 10.90- |
| | 12.40 (m, 2H). one proton is missing |
| Intermediate 587 | 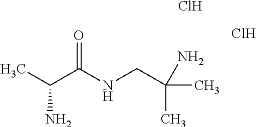 |
| | N-(2-amino-2-methylpropyl)-D-alaninamide-hydrogen chloride (1/2) |
| | ¹H-NMR (400 MHz, DMSO-d₆): δ [ppm] = 1.23 (s, 3H), 1.24 (s, 3H), 1.38 (d, |
| | 3H), 3.15-3.21 (m, 1H), 3.42 (dd, 1H), 3.89 (q, 1H), 8.22 (br s, 6H), 8.93 (t, |
| | 1H). |
| Intermediate 588 | 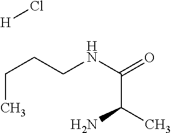 |
| | N-butyl-D-alaninamide-hydrogen chloride (1/1) |
| | ¹H-NMR (400 MHz, DMSO-d₆): δ [ppm] = 0.84-0.90 (m, 3H), 1.21-1.35 (m, |
| | 5H), 1.36-1.45 (m, 2H), 3.02-3.17 (m, 2H), 3.73-3.83 (m, 1H), 8.23 (br s, |
| | 3H), 8.50 (br t, 1H). |
| Intermediate 589 | 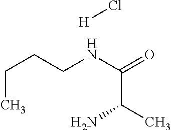 |
| | N-butyl-L-alaninamide-hydrogen chloride (1/1) |
| | ¹H-NMR (400 MHz, DMSO-d₆): δ [ppm] = 0.88 (t, 3H), 1.24-1.35 (m, 5H), 1.36- |
| | 1.45 (m, 2H), 3.03-3.18 (m, 2H), 3.77 (q, 1H), 8.13 (br s, 3H), 8.39 (br t, |
| | 1H). |
| Intermediate 590 | 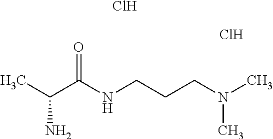 |
| | N-[3-(dimethylamino)propyl]-D-alaninamide-hydrogen chloride (1/2) |
| | ¹H-NMR (400 MHz, DMSO-d₆): δ [ppm] = 1.35 (d, 3H), 1.74-1.91 (m, 2H), 2.72 |
| | (d, 6H), 2.99-3.26 (m, 4H), 3.76-3.86 (m, 1H), 8.31 (br s, 3H), 8.78 (t, 1H), |
| | 10.60 (br s, 1H). |
| Intermediate 591 | 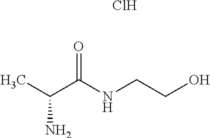 |
| | N-(2-hydroxyethyl)-D-alaninamide-hydrogen chloride (1/1) |
| | ¹H-NMR (400 MHz, DMSO-d₆): δ [ppm] = 1.34 (d, 3H), 3.17 (q, 2H), 3.40-3.46 |
| | (m, 2H), 3.79 (br q, 1H), 4.82 (br s, 1H), 8.19 (br s, 3H), 8.52 (br t, 1H). |

### Intermediate 592

### ethyl 1-cyclopropyl-1H-pyrazole-4-carboxylate

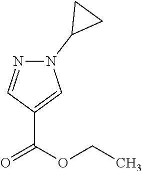

A suspension of ethyl 1H-pyrazole-4-carboxylate (1.50 g, 10.7 mmol), cyclopropylboronic acid (1.84 g, 21.4 mmol) and sodium carbonate (2.27 g, 21.4 mmol) in 1,2-dichloroethane (75 mL) was heated to 70° C. and then 2,2′-bipyridine (1.67 g, 10.7 mmol) and copper(II) acetate (1.94 g, 10.7 mmol) was added. Oxygen was passed through this mixture while stirring at 70° C. for 18 h. After cooling the reaction mixture to rt it was poured into water (100 mL) and extracted three times with dichloromethane. The combined organic phases were washed with brine, filtered using a hydrophobic phase separation filter paper and concentrated under reduced pressure. The residue was purified by flash chromatography to obtain 1.16 g (89% purity, 53% yield) of the target compound.

LC-MS (Method 1): Rₜ=0.90 min; MS (ESIpos): m/z=181 [M+H]⁺

¹H-NMR (400 MHz, DMSO-d₆): δ [ppm]=0.94-1.00 (m, 2H), 1.06-1.11 (m, 2H), 1.25 (t, 3H), 3.80 (tt, 1H), 4.20 (q, 2H), 7.81 (d, 1H), 8.37 (d, 1H).

### Intermediate 593

### ethyl 1-ethyl-3,5-dimethyl-1H-pyrazole-4-carboxylate

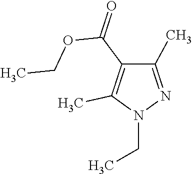

To a stirred solution of ethyl 3,5-dimethyl-1H-pyrazole-4-carboxylate (1.00 g, 5.95 mmol) in DMF (8.3 mL) was added sodium hydride (309 mg, 60% purity, 7.73 mmol) at 0° C. and stirred for 15 minutes. Then bromoethane (530 μL, 7.1 mmol) was added and the reaction mixture was stirred for 30 minutes at rt. Then aqueous sodium hydrogencarbonate solution was added and this mixture was extracted twice with EtOAc. The combined organic layer were washed with brine, filtered using a hydrophobic phase separation filter paper and then concentrated under reduced pressure. The residue was purified by flash chromatography to give 1.05 g (97% purity, 87% yield) of the target compound.

LC-MS (Method 1): Rₜ=1.00 min; MS (ESIpos): m/z=197 [M+H]⁺

¹H-NMR (400 MHz, DMSO-d6) δ[ppm]=1.26 (t, 3H), 1.26 (t, 3H), 2.27 (s, 3H), 2.45 (s, 3H), 4.01 (q, 2H), 4.18 (q, 2H).

### Intermediate 594

### ethyl 1-cyclobutyl-1H-pyrazole-4-carboxylate

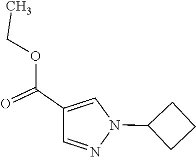

Under argon to 1-cyclobutyl-1H-pyrazole-4-carboxylic acid (1.00 g, 6.02 mmol/in a second experiment 2.50 g, 15.0 mmol were used, CAS [1349718-35-9], commercially available at e.g. Enamine) was added carefully thionyl dichloride (2.52 mL, 34.6 mmol/6.3 mL, 87 mmol) and stirred 15 minutes at rt. Then ethanol (14.4 mL/36 mL) was added dropwise carefully under ice cooling to the mixture and stirred at 0° C. for 30 minutes and then heated at reflux for 1 h. The mixture was poured into a saturated aqueous solution of sodium hydrogencarbonate (50 mL/150 mL) and extracted three times with EtOAc. The combined organic layer were washed with brine, filtered using a hydrophobic phase separation filter paper and then concentrated under reduced pressure. The residue of the two experiments were combined and purified by flash chromatography to give 3.97 g (97% purity, 94% yield) of the target compound.

LC-MS (Method 1): Rₜ=1.03 min; MS (ESIpos): m/z=195 [M+H]⁺

¹H-NMR (400 MHz, DMSO-d6) δ[ppm]=1.25 (t, 3H), 1.70-1.84 (m, 2H), 2.30-2.49 (m, 4H), 4.20 (q, 2H), 4.88 (quin, 1H), 7.87 (s, 1H), 8.39 (s, 1H).

### Intermediate 595

### ethyl 1-(cyclopropylmethyl)-1H-pyrazole-4-carboxylate

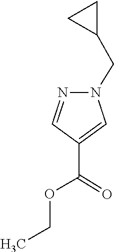

To a stirred solution of ethyl 1H-pyrazole-4-carboxylate (4.03 g, 28.8 mmol) in DMF (40 mL) was added sodium hydride (1.80 g, 50% purity, 37.4 mmol) at 0° C. and stirred for 15 minutes. Then (bromomethyl)cyclopropane (3.2 mL, 35 mmol) was added and the reaction mixture was stirred for 3 days at rt. Then the reaction mixture was poured carefully into icewater and this mixture was extracted twice with EtOAc. The combined organic layer were washed with brine, filtered using a hydrophobic phase separation filter paper and then concentrated under reduced pressure. The residue was purified by flash chromatography to give 3.24 g (95% purity, 55% yield) of the target compound.

LC-MS (Method 1): Rₜ=0.99 min; MS (ESIpos): m/z=195 [M+H]⁺

¹H-NMR (400 MHz, DMSO-d6) δ[ppm]=0.34-0.40 (m, 2H), 0.48-0.57 (m, 2H), 1.20-1.31 (m, 4H), 3.99 (d, 2H), 4.21 (q, 2H), 7.84 (s, 1H), 8.34 (s, 1H).

### Intermediate 596

### ethyl 1-cyclopropyl-3-(difluoromethyl)-1H-pyrazole-4-carboxylate

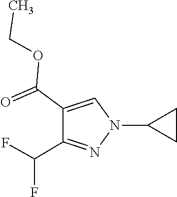 [SMILES: CCOC(=O)c1cn(C2CC2)nc1C(F)F]

Under argon to 1-cyclopropyl-3-(difluoromethyl)-1H-pyrazole-4-carboxylic acid (1.00 g, 4.95 mmol, CAS [2137729-16-7], commercially available at e.g. Enamine) was added carefully thionyl dichloride (2.1 mL, 28 mmol) and stirred 15 minutes at rt. Then ethanol (12 mL) was added carefully dropwise under ice cooling to the mixture and stirred at 0° C. for 30 minutes and then heated at reflux for 4 h and at 90° C. overnight. The mixture was poured into a saturated aqueous solution of sodium hydrogencarbonate (150 mL) and extracted three times with EtOAc. The combined organic layer were washed with brine, filtered using a hydrophobic phase separation filter paper and then concentrated under reduced pressure. The residue was purified by flash chromatography to give 972 mg (95% purity, 81% yield) of the target compound.

LC-MS (Method 1): Rₜ=1.06 min; MS (ESIpos): m/z=231 [M+H]⁺

¹H-NMR (500 MHz, DMSO-d6) δ[ppm]=0.98-1.04 (m, 2H), 1.11-1.15 (m, 2H), 1.27 (t, 3H), 3.87 (tt, 1H), 4.23 (q, 2H), 7.17 (t, 1H), 8.51 (s, 1H).

### Intermediate 597

### methyl 3-methyl-1-(propan-2-yl)-1H-pyrazole-4-carboxylate

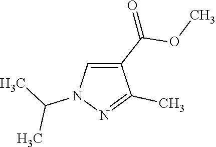

To a solution of 3-methyl-1-(propan-2-yl)-1H-pyrazole-4-carboxylic acid (5.00 g, 29.7 mmol, CAS [113100-42-8], commercially available at e.g. Fluorochem Limited) in acetone (71 mL) was added potassium carbonate (8.22 g, 59.5 mmol) and iodomethane (2.2 mL, 36 mmol). This mixture was stirred for 18 h at rt. After filtration the organic phase was concentrated under reduced pressure. The residue was purified by flash chromatography to give 3.64 g (100% purity, 67% yield) of the target compound.

LC-MS (Method 1): Rₜ=0.92 min; MS (ESIpos): m/z=183 [M+H]+

¹H-NMR (400 MHz, DMSO-d6) δ[ppm]=1.38 (d, 6H), 2.32 (s, 3H), 3.71 (s, 3H), 4.45 (spt, 1H), 8.22 (s, 1H).

### Intermediate 598

### ethyl 1-cyclopropyl-3-ethyl-1H-pyrazole-4-carboxylate

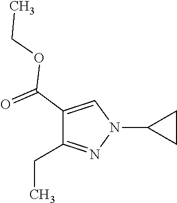

Under argon to 1-cyclopropyl-3-ethyl-1H-pyrazole-4-carboxylic acid (1.00 g, 5.55 mmol, CAS [2138201-37-1], commercially available at e.g. Enamine) was added carefully thionyl dichloride (2.3 mL, 32 mmol) and stirred 15 minutes at rt. Then ethanol (13 mL) was added carefully dropwise under ice cooling to the mixture and stirred at 0° C. for 30 minutes and then heated at reflux for 3 h. The mixture was poured into a saturated aqueous solution of sodium hydrogencarbonate (100 mL) and extracted three times with EtOAc. The combined organic layer were washed with brine, filtered using a hydrophobic phase separation filter paper and then concentrated under reduced pressure. The residue was purified by flash chromatography to give 1.02 g (99% purity, 87% yield) of the target compound.

LC-MS (Method 1): Rₜ=1.09 min; MS (ESIpos): m/z=209 [M+H]⁺

¹H-NMR (400 MHz, DMSO-d6) δ[ppm]=0.90-0.96 (m, 2H), 1.03-1.08 (m, 2H), 1.14 (t, 3H), 1.25 (t, 3H), 2.74 (q, 2H), 3.70 (tt, 1H), 4.18 (q, 2H), 8.20 (s, 1H).

### Intermediate 599

### ethyl 1-(difluoromethyl)-1H-pyrazole-4-carboxylate

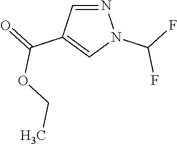

Ethyl 1H-pyrazole-4-carboxylate (3.24 g, 23.1 mmol) and sodium hydrogen carbonate (5.83 g, 69.4 mmol) were added to a stirred solution of sodium chloro(difluoro)acetate (10.6 g, 69.4 mmol) in DMF (12 mL). The reaction mixture was stirred 16 h at 100° C. DMF (6.0 mL) and sodium chloro(difluoro)acetate (7.05 g, 46.2 mmol) were added again to the mixture and the reaction was stirred 24 h at 100° C. The reaction mixture was cooled to rt and diluted with water and EtOAc. The suspension was filtered and the phases separated. The aqueous layer was extracted with EtOAc. The combined organic phases were washed with aqueous saturated sodium chloride solution, dried over sodium sulfate and concentrated under reduced pressure to give 3.25 g of a mixture of the title compound and the starting material (pyrazole). The crude was used without further purification.

LC-MS (method 2): Rₜ=0.90 min; MS (ESIpos): m/z=191 [M+H]+

### Intermediate 600

### 1-cyclopropyl-1H-pyrazole-4-carbohydrazide

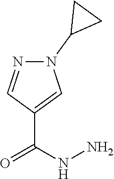 [SMILES: NNC(=O)c1cnn(C2CC2)c1]

Ethyl 1-cyclopropyl-1H-pyrazole-4-carboxylate (2.36 g, 13.1 mmol) was solubilised in ethanol (17 mL), hydrazine monohydrate (5.3 mL, 60% purity, 65 mmol) was added and the mixture was stirred for 18 h at 110° C. The reaction mixture was cooled to rt then cooled to 0-5° C. The formed solid was collected by filtration and dried to obtain 2.12 g (100% purity, 97% yield) of the title compound that was used without further purification.

LC-MS (Method 1): Rₜ=65.00 min; MS (ESIpos): m/z=167 [M+H]⁺

¹H-NMR (400 MHz, DMSO-d₆): δ [ppm]=0.93-0.99 (m, 2H), 1.00-1.05 (m, 2H), 3.71-3.77 (m, 1H), 4.31 (br s, 2H), 7.79 (d, 1H), 8.18 (d, 1H), 9.28 (s, 1H).

### Intermediate 601

### 1-ethyl-3,5-dimethyl-1H-pyrazole-4-carbohydrazide

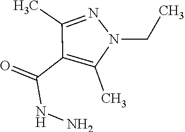

Ethyl 1-ethyl-3,5-dimethyl-1H-pyrazole-4-carboxylate (1.05 g, 5.35 mmol) was solubilised in ethanol (6.7 mL), hydrazine hydrate (2.2 mL, 60% purity, 27 mmol) was added and the mixture was stirred for 3 days at 110° C. After cooling to rt aqueous saturated ammonium chloride solution and ethyl acetate was added. After separation of the organic phase the aqueous phase was extracted three times with ethyl acetate and finally with dichloromethane. The combined organic phases were filtered using a hydrophobic phase separation filter paper and then concentrated under reduced pressure. After drying 660 mg (90% purity, 61% yield) of the target compound was obtained, which was used without further purification.

LC-MS (Method 1): Rₜ=0.46 min; MS (ESIpos): m/z=183 [M+H]⁺

¹H-NMR (400 MHz, DMSO-d6) δ[ppm]=1.24 (t, 3H), 2.19 (s, 3H), 2.31 (s, 3H), 3.96 (q, 2H), 4.32 (br s, 2H), 8.70 (s, 1H).

### Intermediate 602

### 1-cyclobutyl-1H-pyrazole-4-carbohydrazide

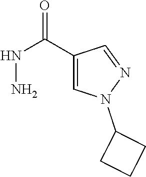 [SMILES: NNC(=O)c1cnn(C2CCC2)c1]

Ethyl 1-cyclobutyl-1H-pyrazole-4-carboxylate (3.97 g, 20.4 mmol) was solubilised in toluene (26 mL), hydrazine hydrate (8.3 mL, 60% purity, 100 mmol) was added and the mixture was stirred for 2 days at 50° C. After cooling to rt the reaction mixture was diluted with ethyl acetate and extracted with saturated aqueous ammonium chloride solution. After separation of the organic phase the aqueous phase was extracted twice with ethyl acetate. The combined organic phases were washed with brine, filtered using a hydrophobic phase separation filter paper and then concentrated under reduced pressure. After drying 1.70 g (95% purity, 44% yield) of the target compound was obtained, which was used without further purification.

LC-MS (Method 2): Rₜ=0.55 min; MS (ESIpos): m/z=181 [M+H]⁺

¹H-NMR (400 MHz, DMSO-d6) δ[ppm]=1.70-1.83 (m, 2H), 2.31-2.48 (m, 4H), 4.32 (s, 2H), 4.83 (quin, 1H), 7.86 (s, 1H), 8.21 (s, 1H), 9.30 (s, 1H).

### Intermediate 603

### 1-(cyclopropylmethyl)-1H-pyrazole-4-carbohydrazide

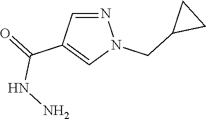 [SMILES: NNC(=O)c1cnn(CC2CC2)c1]

Ethyl 1-(cyclopropylmethyl)-1H-pyrazole-4-carboxylate (3.24 g, 16.7 mmol) was solubilised in toluene (21 mL), hydrazine hydrate (10 mL, 60% purity, 130 mmol) was added and the mixture was stirred for 2 days at 50° C. After cooling to rt a solid was formed. The reaction mixture was filtered and the solid was washed with toluene. After drying of the solid 2.24 g (95% purity, 71% yield) of the target compound was obtained, which was used without further purification.

LC-MS (Method 2): Rₜ=0.56 min; MS (ESIpos): m/z=181 [M+H]⁺

¹H-NMR (400 MHz, DMSO-d6) δ[ppm]=0.33-0.39 (m, 2H), 0.49-0.56 (m, 2H), 1.17-1.27 (m, 1H), 3.96 (d, 2H), 4.35 (br s, 2H), 7.82 (d, 1H), 8.18 (d, 1H), 9.32 (s, 1H).

### Intermediate 604

### 1-cyclopropyl-3-(difluoromethyl)-1H-pyrazole-4-carbohydrazide

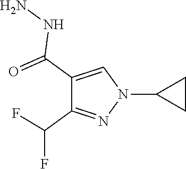 [SMILES: NNC(=O)c1cn(C2CC2)nc1C(F)F]

Ethyl 1-cyclopropyl-3-(difluoromethyl)-1H-pyrazole-4-carboxylate (972 mg, 4.22 mmol) was solubilised in toluene (5.3 mL), hydrazine hydrate (1.7 mL, 60% purity, 21 mmol) was added and the mixture was stirred at 70° C. overnight. After cooling to rt a solid was formed. The reaction mixture was filtered and the solid was washed with water. After drying of the solid 789 mg (95% purity, 82% yield) of the target compound was obtained, which was used without further purification.

LC-MS (Method 2): Rₜ=0.59 min; MS (ESIneg): m/z=215 [M−H]⁻.

¹H-NMR (500 MHz, DMSO-d6) δ[ppm]=1.00-1.07 (m, 4H), 3.78-3.88 (m, 1H), 4.43 (br s, 2H), 7.31 (t, 1H), 8.31 (s, 1H), 9.41 (br s, 1H).

### Intermediate 605

### 3-methyl-1-(propan-2-yl)-1H-pyrazole-4-carbohydrazide

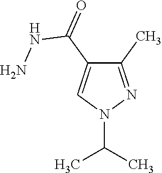

Methyl 3-methyl-1-(propan-2-yl)-1H-pyrazole-4-carboxylate (3.64 g, 20.0 mmol) was solubilised in ethanol (25 mL), hydrazine hydrate (8.1 mL, 60% purity, 100 mmol) was added and the mixture was stirred over night at 110° C. After cooling to rt aqueous saturated ammonium chloride solution, water and ethyl acetate was added. After separation of the organic phase the aqueous phase was extracted twice with ethyl acetate. The combined organic phases were washed with brine, filtered using a hydrophobic phase separation filter paper and then concentrated under reduced pressure. After drying 1.46 g (85% purity, 34% yield) of the target compound was obtained, which was used without further purification.

LC-MS (Method 1): Rₜ=0.50 min; MS (ESIpos): m/z=183 [M+H]⁺

¹H-NMR (400 MHz, DMSO-d6) δ[ppm]=1.37 (d, 6H), 2.31 (s, 3H), 4.24 (br s, 2H), 4.37 (spt, 1H), 8.09 (s, 1H), 9.00 (br s, 1H).

### Intermediate 606

### 1-cyclopropyl-3-ethyl-1H-pyrazole-4-carbohydrazide

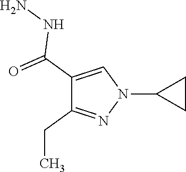

Ethyl 1-cyclopropyl-3-ethyl-1H-pyrazole-4-carboxylate (1.02 g, 4.90 mmol) was solubilised in toluene (6.2 mL), hydrazine hydrate (2.0 mL, 60% purity, 24 mmol) was added and the mixture was stirred at 70° C. overnight, then at 110° C. for 6 days. After cooling to rt a solid was formed. The reaction mixture was filtered and the solid was washed with toluene. After drying of the solid 690 mg (97% purity, 70% yield) of the target compound was obtained, which was used without further purification.

LC-MS (Method 2): Rₜ=0.58 min; MS (ESIpos): m/z=195 [M+H]⁺

¹H-NMR (400 MHz, DMSO-d6) δ[ppm]=0.91-0.99 (m, 4H), 1.11 (t, 3H), 2.75 (q, 2H), 3.59-3.67 (m, 1H), 4.25 (br s, 2H), 8.08 (s, 1H), 9.02 (br s, 1H).

### Intermediate 607 and Intermediate 608

### 1-(difluoromethyl)-1H-pyrazole-4-carbohydrazide and 1H-pyrazole-4-carbohydrazide

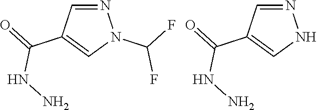

A mixture of ethyl 1-(difluoromethyl)-1H-pyrazole-4-carboxylate and ethyl 1H-pyrazole-4-carboxylate (2.58 g) was solubilised in toluene (20 mL), hydrazine hydrate (5.5 mL, 60% purity) was added and the mixture was stirred 16 h at 50° C. (The mixture was allowed to cool down to rt and the precipitate was filtered. The solid was dried under reduced pressure at 60° C. to give 1.54 g of a mixture of the title compounds. The crude was used without further purification.

### Intermediate 609

### di-tert-butyl (2-bromo-6-cyanophenyl)-2-imidodicarbonate

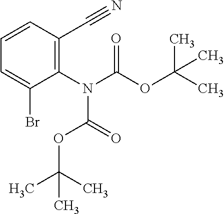 [SMILES: CC(C)(C)OC(=O)N(C(=O)OC(C)(C)C)c1c(Br)cccc1C#N]

2-Amino-3-bromobenzonitrile (2.50 g, 12.7 mmol), di-tert-butyl dicarbonate (11.1 g, 50.8 mmol), (77.5 mg, 634 μmol;) and triethylamine (2.1 mL, 15 mmol) were stirred in THF (75 mL) and the reaction was stirred 18 hours at rt. The mixture was concentrated underreduced pressure and then diluted with water. The aqueous phase was extracted with dichloromethane, dried over sodium sulfate, filtered and concentrated under reduced pressure. The crude mixture was then purified by flash column chromatography to give 4.41 g (95% purity, 83% yield) of the title compound.

¹H-NMR (400 MHz, DMSO-d6): δ [ppm]=1.36 (s, 18H), 7.52 (t, 1H), 8.00 (dd, 1H), 8.11 (dd, 1H).

### Intermediate 610

### di-tert-butyl (2-chloro-6-cyanophenyl)-2-imidodicarbonate

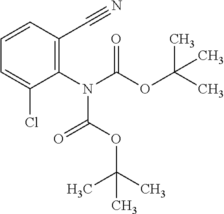 [SMILES: CC(C)(C)OC(=O)N(C(=O)OC(C)(C)C)c1c(Cl)cccc1C#N]

2-Amino-3-chlorobenzonitrile (3.00 g, 19.7 mmol), di-tert-butyl dicarbonate (11 mL, 49 mmol]), N,N-diisopropylethylamine (8.6 mL, 49 mmol) and (1.20 g, 9.83 mmol) were solubilised in 1,4-dioxane (72 mL). The reaction mixture was stirred at rt for 22 h. The reaction mixture was quenched with water and extracted with ethylacetate. The organic phase was washed with water and brine. The organic phase was dried over sodium sulfate, filtered and concentrated under reduced pressure. The crude material was purified by flash column chromatography to give 6.42 g (95% purity, 88% yield) of the title compound.

¹H-NMR (400 MHz, DMSO-d6): δ [ppm]=1.36 (s, 18H), 7.62 (t, 1H), 7.90-8.06 (m, 2H).

### Intermediate 611

### di-tert-butyl [2-cyano-6-(trifluoromethyl)phenyl]-2-imidodicarbonate

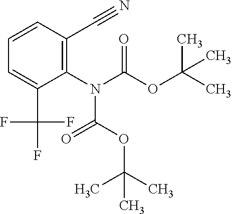 [SMILES: CC(C)(C)OC(=O)N(C(=O)OC(C)(C)C)c1c(C#N)cccc1C(F)(F)F]

To a solution of 2-amino-3-(trifluoromethyl)benzonitrile (2.00 g, 10.7 mmol) in dioxane (48 mL) was added N,N-diisopropylethylamine (4.7 mL, 27 mmol; CAS [7087-68-5]), 4-(N,N-dimethylamino)pyridine (4.7 mL, 27 mmol; CAS [7087-68-5]) and di-tert-butyl dicarbonate (6.2 mL, 27 mmol; CAS [24424-99-5]). This reaction mixture was stirred for 90 h at rt, then concentrated under vacuum. The resulting residue was purified via flash chromatography to obtain 3.89 g (56% yield) of the title compound.

¹H-NMR (400 MHz, DMSO-d₆): δ [ppm]=1.33 (s, 18H), 7.85 (t, 1H), 8.19 (dd, 1H), 8.34 (dd, 1H).

### Intermediate 612

### di-tert-butyl (2-cyano-6-methoxyphenyl)-2-imidodicarbonate

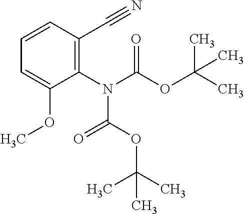 [SMILES: COc1cccc(C#N)c1N(C(=O)OC(C)(C)C)C(=O)OC(C)(C)C]

To a solution of 2-amino-3-methoxybenzonitrile (250 mg g, 1.696 mmol/in a second experiment was used 4.75 g, 32.1 mmol) in dioxane (42 mL/140 mL) was added N,N-diisopropylethylamine (4.2 mL, 24 mmol/14.0 mL, 80.1 mmol), 4-(N,N-dimethylamino)pyridine (584 mg, 4.78 mmol/1.96 g, 16.0 mmol) and di-tert-butyl dicarbonate (5.5 mL, 24 mmol/18.4, 80.1 mmol; CAS [24424-99-5]). This reaction mixture was stirred for 20 h at rt, then concentrated under vacuum. The combined resulting residues were purified via flash chromatography to obtain 8.89 g (76% yield) of the title compound.

¹H-NMR (400 MHz, DMSO-d₆): δ [ppm]=1.35 (s, 18H), 3.86 (s, 3H), 7.44-7.49 (m, 2H), 7.53 (dd, 1H).

### Intermediate 613

### di-tert-butyl [2-cyano-6-(trifluoromethoxy)phenyl]-2-imidodicarbonate

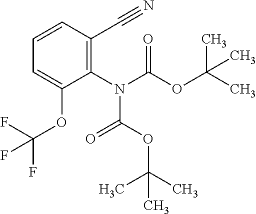 [SMILES: CC(C)(C)OC(=O)N(C(=O)OC(C)(C)C)c1c(C#N)cccc1OC(F)(F)F]

To a solution of 2-amino-3-(trifluoromethoxy)benzonitrile (250 mg, 1.24 mmol; CAS [1261581-55-8], e.g. Ark Pharm, Inc.) in dioxane (5.5 mL) was added N,N-diisopropylethylamine (540 μL, 3.1 mmol), 4-(N,N-dimethylamino)pyridine (75.5 mg, 618 μmol) and di-tert-butyl dicarbonate (710 μL, 3.1 mmol). This reaction mixture was stirred for 20 h at rt, then concentrated under vacuum. The combined resulting residues were purified via flash chromatography to obtain 452 mg (91% yield) of the title compound.

¹H-NMR (400 MHz, DMSO-d₆): δ [ppm]=1.36 (s, 18H), 7.74 (dd, 1H), 7.92 (ddq, 1H), 8.04 (dd, 1H).

### Intermediate 614

### di-tert-butyl (2,6-dicyanophenyl)-2-imidodicarbonate

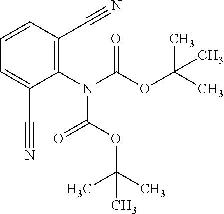 [SMILES: CC(C)(C)OC(=O)N(C(=O)OC(C)(C)C)c1c(C#N)cccc1C#N]

To a solution of 2-aminobenzene-1,3-dicarbonitrile (3.40 g, 23.8 mmol; CAS [63069-52-3], e.g. ABCR) in dioxane (110 mL) was added N,N-diisopropylethylamine (10 mL, 59 mmol), 4-(N,N-dimethylamino)pyridine (1.45 g, 11.9 mmol) and di-tert-butyl dicarbonate (14 mL, 59 mmol). This reaction mixture was stirred for one day at rt, then concentrated under vacuum. The resulting residue was purified via flash chromatography to obtain 7.80 g (95% purity, 91% yield) of the title compound.

LC-MS (Method 1): Rₜ=1.29 min; MS (ESIpos): m/z=244 [M+H]⁺

¹H-NMR (400 MHz, DMSO-d₆): δ [ppm]=1.38 (s, 18H), 7.83 (t, 1H), 8.36 (d, 2H).

### Intermediate 615

### di-tert-butyl (2-cyano-6-cyclopropylphenyl)-2-imidodicarbonate

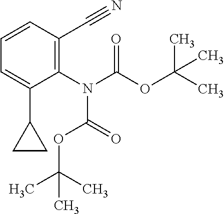 [SMILES: CC(C)(C)OC(=O)N(C(=O)OC(C)(C)C)c1c(C#N)cccc1C1CC1]

2-Amino-3-cyclopropylbenzonitrile (309 mg, 1.95 mmol), di-tert-butyl dicarbonate (1.28 g, 5.85 mmol) and DMAP (119 mg, 975 μmol) were solubilised in 1,4-dioxane (24 mL) and N,N-diisopropylethylamine (850 μl, 4.9 mmol) was added. The reaction was stirred for 24 h at rt. di-tert-butyl dicarbonate (1.28 g, 5.85 mmol) and N,N-diisopropylethylamine (850 μl, 4.9 mmol) were added again to the mixture and the reaction was stirred for another 4 h. The reaction mixture was diluted with water and EtOAc. The aqueous phase was extracted with EtOAc. The organic phase was dried with sat. aq. NaCl, dried (silicon filter) and concentrated under reduced pressure. The crude mixture was purified by flash column chromatography to give 438 mg (65% purity, 41% yield) of the title compound.

LC-MS (method 1): Rₜ=1.39 min; MS (ESIpos): m/z=358 [M+H]+

1H-NMR (400 MHz, DMSO-d6): δ [ppm]=0.65-0.73 (m, 2H), 0.94-1.03 (m, 2H), 1.36 (s, 18H), 1.70-1.81 (m, 1H), 7.34 (dd, 1H), 7.45 (t, 1H), 7.72 (dd, 1H).

### Intermediate 616

### di-tert-butyl [2-cyano-6-(dimethylamino)phenyl]-2-imidodicarbonate

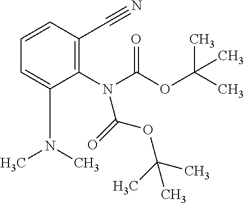 [SMILES: CN(C)c1cccc(C#N)c1N(C(=O)OC(C)(C)C)C(=O)OC(C)(C)C]

2-Amino-3-(dimethylamino)benzonitrile (140 mg, 868 μmol), di-tert-butyl dicarbonate (800 μL, 3.5 mmol), DMAP (5.30 mg, 43.4 μmol) and triethylamine (150 μl, 1.0 mmol) were stirred in THF (5.1 mL) for 48 h at 50° C. The mixture was cooled to rt, concentrated and then diluted with H₂O. The aqueous phase was washed with DCM and the organic phase was dried (silicon filter) and concentrated under reduced pressure. The crude mixture was purified by flash column chromatography to give 189 mg (95% purity, 57% yield) of the title compound.

LC-MS (method 2): Rₜ=1.39 min; MS (ESIpos): m/z=361 [M]⁺

1H-NMR (400 MHz, DMSO-d6): δ [ppm]=1.36 (s, 18H), 2.69 (s, 6H), 7.37-7.41 (m, 1H), 7.42-7.45 (m, 2H).

### Intermediate 617

### di-tert-butyl (2-cyano-6-fluorophenyl)-2-imidodicarbonate

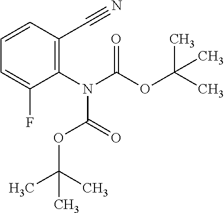 [SMILES: CC(C)(C)OC(=O)N(C(=O)OC(C)(C)C)c1c(F)cccc1C#N]

2-Amino-3-fluorobenzonitrile (2.00 g, 14.7 mmol), di-tert-butyl dicarbonate (10 mL, 44 mmol) and DMAP (89.7 mg, 735 μmol) was dissolved in THF (80 mL) and stirred for 48 h at rt. Water was added to the mixture and the organic phase was extracted with DCM. The organic phase was dried (silicon filter) and concentrated under reduce pressure. The crude mixture was purified by flash chromatography to give 4.88 g (98% purity, 97% yield) of the title compound.

LC-MS (method 2): Rₜ=1.33 min; MS (ESIpos): m/z=336 [M]+

¹H-NMR (400 MHz, DMSO-d6): δ [ppm]=1.38 (s, 18H), 7.62-7.69 (m, 1H), 7.79 (ddd, 1H), 7.84 (dt, 1H).

### Intermediate 618

### ethyl [2-cyano-6-(methylsulfanyl)phenyl]carbamate

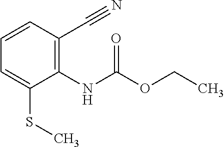 [SMILES: CCOC(=O)Nc1c(C#N)cccc1SC]

Under argon atmosphere, ethyl (2-bromo-6-cyanophenyl)carbamate (1.00 g, 3.72 mmol) was solubilised in dry THF and the mixture was cooled to −78° C. MeLi (2.7 mL, 1.5 M, 4.1 mmol) was added dropwise and the reaction was stirred for 5 minutes. n-BuLi (3.0 mL, 2.2 M, 6.5 mmol) was then added dropwise and the mixture stirred 20 minutes at −78° C. (Methyldisulfanyl)methane (3.5 mL, 37 mmol) was added and the reaction was allowed to warm up to rt over 2 h. The mixture was poured into half saturated aqueous Ammonium chloride solution and extracted three times with EtOAc. The organic layer was dried using sodium sulfate, filtered and concentrated under reduced pressure. The residue was purified by flash column chromatography to give 468 mg (100% purity, 53% yield) of the title compound.

LC-MS (method 2): Rₜ=0.93 min; MS (ESIpos): m/z=237 [M+H]⁺

¹H-NMR (400 MHz, DMSO-d6): δ [ppm]=1.09-1.32 (m, 3H), 2.45 (s, 3H), 4.10 (q, 2H), 7.43-7.52 (t, 1H), 7.59 (dd, 1H), 7.62 (dd, 1H), 9.37 (br s, 1H).

### Intermediate 619

### ethyl {2-cyano-6-[(propan-2-yl)sulfanyl]phenyl}carbamate

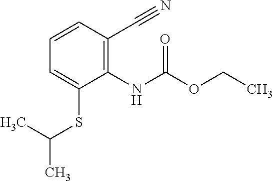 [SMILES: CCOC(=O)Nc1c(C#N)cccc1SC(C)C]

The compound was synthesised similarly to intermediate 618.

LC-MS (method 2): Rₜ=1.11 min; MS (ESIpos): m/z=265 [M+H]⁺

¹H-NMR (400 MHz, DMSO-d6): δ [ppm]=1.20 (br d, 3H), 1.24 (d, 6H), 3.56 (quin, 1H), 4.09 (d, 2H), 7.44 (t, 1H), 7.68 (dd, 1H), 7.73-7.77 (m, 1H), 9.33 (br s, 1H).

### Intermediate 620

### ethyl [2-cyano-6-(ethylsulfanyl)phenyl]carbamate

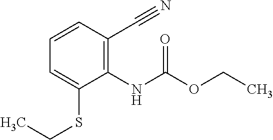 [SMILES: CCOC(=O)Nc1c(C#N)cccc1SCC]

The compound was synthesised similarly to intermediate 618

LC-MS (Method 2): Rₜ=1.03 min; MS (ESIpos): m/z=251 [M+H]⁺

¹H-NMR (400 MHz, DMSO-d6): δ [ppm]=1.15-1.27 (m, 6H), 2.98 (q, 2H), 4.00-4.14 (m, 2H), 7.42-7.47 (m, 1H), 7.65 (ddd, 2H), 9.34 (br s, 1H).

### Intermediate 621

### 2-(1-methyl-1H-pyrazol-4-yl)-7-(trifluoromethyl)[1,2,4]triazolo[1,5-c]quinazolin-5(6H)-one

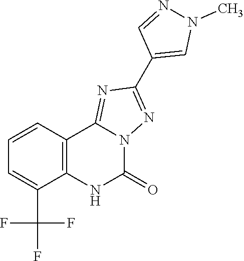 [SMILES: Cn1cc(-c2nc3c4cccc(C(F)(F)F)c4[nH]c(=O)n3n2)cn1]

Di-tert-butyl [2-cyano-6-(trifluoromethyl)phenyl]-2-imidodicarbonate (4.00 g, 10.4 mmol) and 1-methyl-1H-pyrazole-4-carbohydrazide (1.74 g, 12.4 mmol; CAS [170020-91-4]) were stirred in DMF (34 mL) at 120° C. for 20 h. Then acetic acid (35 mL) was added at 100° C. and the reaction mixture was stirred for 24 h at this temperature. The reaction mixture was cooled to room temperature and added to water. After stirring for 10 minutes the solid was obtained by filtration, washed with water and dried to give 3.01 g (100% purity, 87% yield) of the target compound, which was used without further purification.

LC-MS (Method 1): Rₜ=0.88 min; MS (ESIneg): m/z=333 [M−H]⁻.

¹H-NMR (400 MHz, DMSO-d₆): δ [ppm]=3.95 (s, 3H), 7.56 (t, 1H), 8.03 (d, 1H), 8.07 (dd, 1H), 8.43 (d, 1H), 8.49 (d, 1H), 11.53 (br s, 1H).

The following intermediates were prepared similarly to intermediate 621:

| | Structure |
|---|---|
| | IUPAC-Name |
| | LC-MS (method): Retention time; Mass found |
| Example | ¹H-NMR |
| Intermediate 622 | 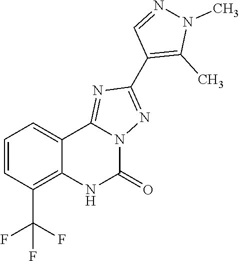 [SMILES: Cc1c(-c2nc3c4cccc(C(F)(F)F)c4[nH]c(=O)n3n2)cnn1C] |
| | 2-(1,5-dimethyl-1H-pyrazol-4-yl)-7-(trifluoromethyl)[1,2,4]triazolo[1,5- |
| | c]quinazolin-5(6H)-one |
| | LC-MS (Method 1): Rₜ = 0.96 min; MS (ESIneg): m/z = 347 [M − H]⁻ |
| | ¹H-NMR (400 MHz, DMSO-d₆): [ppm] = 2.71 (s, 3H), 3.83 (s, 3H), 7.56 (t, |
| | 1H), 7.94 (s, 1H), 8.07 (dd, 1H), 8.52 (dd, 1H), 11.54 (br s, 1H). |
| Intermediate 623 | 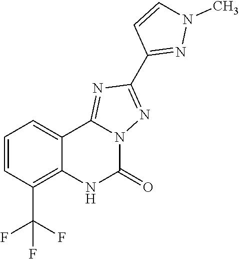 [SMILES: Cn1ccc(-c2nc3c4cccc(C(F)(F)F)c4[nH]c(=O)n3n2)n1] |
| | 2-(1-methyl-1H-pyrazol-3-yl)-7-(trifluoromethyl)[1,2,4]triazolo[1,5- |
| | c]quinazolin-5(6H)-one |
| | LC-MS (Method 1): Rₜ = 0.87 min; MS (ESIneg): m/z = 333 [M − H]⁻ |
| | ¹H-NMR (400 MHz, DMSO-d₆): δ [ppm] = 3.97 (s, 3H), 6.87 (d, 1H), 7.57 (t, |
| | 1H), 7.88 (d, 1H), 8.08 (d, 1H), 8.53 (d, 1H), 11.56 (br s, 1H). |
| Intermediate 624 | 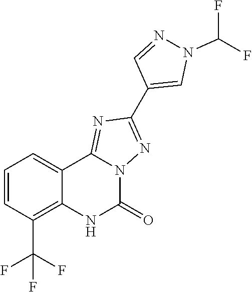 [SMILES: O=c1[nH]c2c(C(F)(F)F)cccc2c2nc(-c3cnn(C(F)F)c3)nn12] |
| | 2-[1-(difluoromethyl)-1H-pyrazol-4-yl]-7- |
| | (trifluoromethyl)[1,2,4]triazolo[1,5-c]quinazolin-5(6H)-one |
| | LC-MS (Method 1): Rₜ = 0.98 min; MS (ESIneg): m/z = 369 [M − H]⁻ |
| | ¹H-NMR (400 MHz, DMSO-d₆): δ [ppm] = 7.56 (t, 1H), 8.08 (dd, 1H), 8.10 (br |
| | s, 1H), 8.45 (br s, 1H), 8.51 (dd, 1H), 11.53 (br s, 1H), 13.35 (br s, 1H). |
| Intermediate 625 | 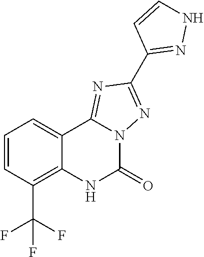 [SMILES: O=c1[nH]c2c(C(F)(F)F)cccc2c2nc(-c3cc[nH]n3)nn12] |
| | 2-(1H-pyrazol-3-yl)-7-(trifluoromethyl)[1,2,4]triazolo[1,5-c]quinazolin- |
| | 5(6H)-one |
| | LC-MS (Method 1): Rₜ = 0.83 min; MS (ESIneg): m/z = 319 [M − H]⁻ |
| | ¹H-NMR (400 MHz, DMSO-d₆): δ [ppm] = 6.91 (br s, 1H), 7.58 (t, 1H), 7.86- |
| | 7.99 (m, 1H), 8.09 (d, 1H), 8.54 (d, 1H), 11.58 (br s, 1H), 13.35 (br s, 1H). |
| Intermediate 626 | 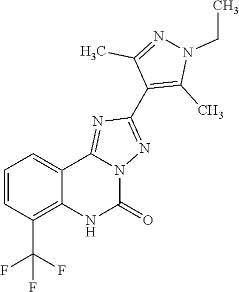 [SMILES: CCn1nc(C)c(-c2nc3c4cccc(C(F)(F)F)c4[nH]c(=O)n3n2)c1C] |
| | 2-(1-ethyl-3,5-dimethyl-1H-pyrazol-4-yl)-7- |
| | (trifluoromethyl)[1,2,4]triazolo[1,5-c]quinazolin-5(6H)-one |
| | LC-MS (Method 1): Rₜ = 1.10 min; MS (ESIpos): m/z = 377 [M + H]⁺ |
| | ¹H-NMR (400 MHz, DMSO-d₆): δ [ppm] = 1.33 (t, 3H), 2.50-2.51 (m, 3H, |
| | methyl group in DMSO signal), 2.70 (s, 3H), 4.09 (q, 2H), 7.55 (br t, 1H), |
| | 8.06 (d, 1H), 8.52 (d, 1H), 11.54 (br s, 1H). |
| Intermediate 627 | 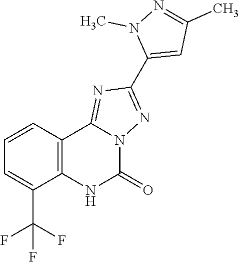 [SMILES: Cc1cc(-c2nc3c4cccc(C(F)(F)F)c4[nH]c(=O)n3n2)n(C)n1] |
| | 2-(1,3-dimethyl-1H-pyrazol-5-yl)-7-(trifluoromethyl)[1,2,4]triazolo[1,5- |
| | c]quinazolin-5(6H)-one |
| | LC-MS (Method 1): Rₜ = 1.03 min; MS (ESIpos): m/z = 349 [M + H]⁺ |
| | ¹H-NMR (400 MHz, DMSO-d₆): δ [ppm] = 2.23 (s, 3H), 4.21 (s, 3H), 6.78 (s, |
| | 1H), 7.58 (t, 1H), 8.10 (d, 1H), 8.53 (d, 1H), 11.71 (br s, 1H). |
| Intermediate 628 | 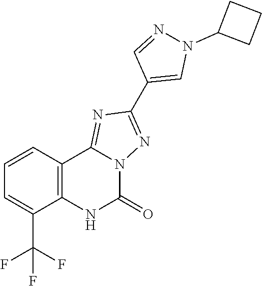 [SMILES: O=c1[nH]c2c(C(F)(F)F)cccc2c2nc(-c3cnn(C4CCC4)c3)nn12] |
| | 2-(1-cyclobutyl-1H-pyrazol-4-yl)-7-(trifluoromethyl)[1,2,4]triazolo[1,5- |
| | c]quinazolin-5(6H)-one |
| | LC-MS (Method 1): Rₜ = 1.05 min; MS (ESIpos): m/z = 375 [M + H]⁺ |
| | ¹H-NMR (500 MHz, DMSO-d₆): δ [ppm] = 1.77-1.87 (m, 2H), 2.39-2.46 (m, |
| | 2H), 2.52-2.59 (m, 3H), 4.93-5.01 (m, 1H), 7.57 (t, 1H), 8.06-8.10 (m, |
| | 2H), 8.50 (dd, 1H), 8.55 (d, 1H), 11.54 (br s, 1H). |
| Intermediate 629 | 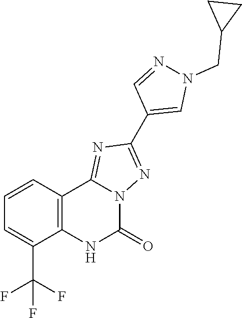 [SMILES: O=c1[nH]c2c(C(F)(F)F)cccc2c2nc(-c3cnn(CC4CC4)c3)nn12] |
| | 2-[1-(cyclopropylmethyl)-1H-pyrazol-4-yl]-7- |
| | (trifluoromethyl)[1,2,4]triazolo[1,5-c]quinazolin-5(6H)-one |
| | LC-MS (Method 1): Rₜ = 1.03 min; MS (ESIpos): m/z = 375 [M + H]⁺ |
| | ¹H-NMR (500 MHz, DMSO-d₆): δ [ppm] = 0.41-0.46 (m, 2H), 0.55-0.59 (m, |
| | 2H), 1.27-1.36 (m, 1H), 4.08 (d, 2H), 7.56 (t, 1H), 8.05 (d, 1H), 8.08 (d, |
| | 1H), 8.48-8.52 (m, 2H), 11.54 (br s, 1H). |
| Intermediate 630 | 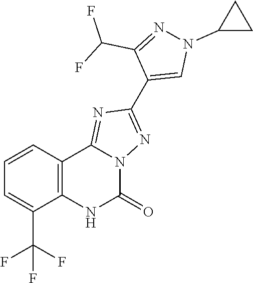 [SMILES: O=c1[nH]c2c(C(F)(F)F)cccc2c2nc(-c3cn(C4CC4)nc3C(F)F)nn12] |
| | 2-[1-cyclopropyl-3-(difluoromethyl)-1H-pyrazol-4-yl]-7- |
| | (trifluoromethyl)[1,2,4]triazolo[1,5-c]quinazolin-5(6H)-one |
| | LC-MS (Method 1): Rₜ = 1.11 min; MS (ESIpos): m/z = 411 [M + H]⁺ |
| | ¹H-NMR (400 MHz, DMSO-d₆): δ [ppm] = 1.03-1.10 (m, 2H), 1.18-1.24 (m, |
| | 2H), 3.96 (tt, 1H), 7.58 (t, 1H), 7.58 (t, 1H), 8.09 (dd, 1H), 8.52 (dd, 1H), |
| | 8.66 (br s, 1H), 11.63 (br s, 1H). |
| Intermediate 631 | 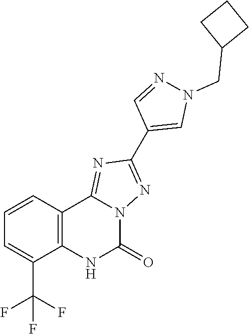 [SMILES: O=c1[nH]c2c(C(F)(F)F)cccc2c2nc(-c3cnn(CC4CCC4)c3)nn12] |
| | 2-[1-(cyclobutylmethyl)-1H-pyrazol-4-yl]-7- |
| | (trifluoromethyl)[1,2,4]triazolo[1,5-c]quinazolin-5(6H)-one |
| | LC-MS (Method 1): Rₜ = 1.14 min; MS (ESIpos): m/z = 389 [M + H]⁺ |
| | ¹H-NMR (400 MHz, DMSO-d₆): δ [ppm] = 1.76-1.91 (m, 4H), 1.94-2.04 (m, |
| | 2H), 2.76-2.88 (m, 1H), 4.24 (d, 2H), 7.56 (t, 1H), 8.04 (d, 1H), 8.08 (dd, |
| | 1H), 8.44 (d, 1H), 8.50 (dd, 1H), 11.53 (br s, 1H). |
| Intermediate 632 | 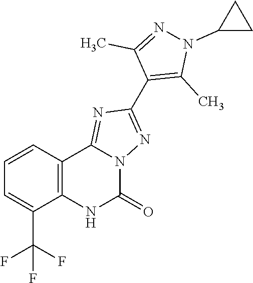 [SMILES: Cc1nn(C2CC2)c(C)c1-c1nc2c3cccc(C(F)(F)F)c3[nH]c(=O)n2n1] |
| | 2-(1-cyclopropyl-3,5-dimethyl-1H-pyrazol-4-yl)-7- |
| | (trifluoromethyl)[1,2,4]triazolo[1,5-c]quinazolin-5(6H)-one |
| | LC-MS (Method 1): Rₜ = 1.11 min; MS (ESIpos): m/z = 389 [M + H]⁺ |
| | ¹H-NMR (400 MHz, DMSO-d₆): δ [ppm] = 0.98-1.11 (m, 4H), 2.48 (s, 3H), |
| | 2.77 (s, 3H), 3.53 (tt, 1H), 7.55 (br t, 1H), 8.07 (d, 1H), 8.52 (d, 1H), 11.54 |
| | (br s, 1H). |
| Intermediate 633 | 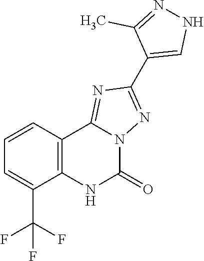 [SMILES: Cc1n[nH]cc1-c1nc2c3cccc(C(F)(F)F)c3[nH]c(=O)n2n1] |
| | 2-(3-methyl-1H-pyrazol-4-yl)-7-(trifluoromethyl)[1,2,4]triazolo[1,5- |
| | c]quinazolin-5(6H)-one |
| | LC-MS (Method 1): Rₜ = 0.88 min; MS (ESIpos): m/z = 335 [M + H]⁺ |
| | ¹H-NMR (400 MHz, DMSO-d₆): δ [ppm] = 2.63 (br s, 3H), 7.56 (t, 1H), 7.96- |
| | 8.14 (m, 2H), 8.51 (d, 1H), 11.53 (br s, 1H), 13.01 (br s, 1H). |
| Intermediate 634 | 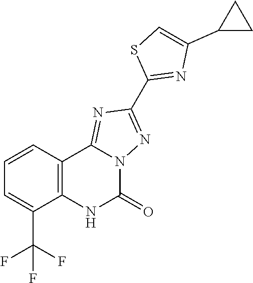 [SMILES: O=c1[nH]c2c(C(F)(F)F)cccc2c2nc(-c3nc(C4CC4)cs3)nn12] |
| | 2-(4-cyclopropyl-1,3-thiazol-2-yl)-7-(trifluoromethyl)[1,2,4]triazolo[1,5- |
| | c]quinazolin-5(6H)-one |
| | LC-MS (Method 1): Rₜ = 1.15 min; MS (ESIpos): m/z = 378 [M + H]⁺ |
| | ¹H-NMR (400 MHz, DMSO-d₆): δ [ppm] = 0.91-1.03 (m, 4H), 2.18-2.26 (m, |
| | 1H), 7.58 (t, 1H), 7.60 (s, 1H), 8.11 (dd, 1H), 8.56 (dd, 1H), 11.75 (br s, 1H). |
| Intermediate 635 | 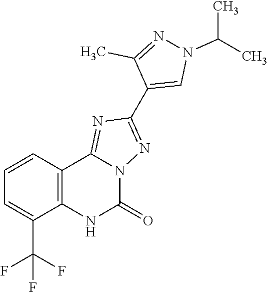 [SMILES: Cc1nn(C(C)C)cc1-c1nc2c3cccc(C(F)(F)F)c3[nH]c(=O)n2n1] |
| | 2-[3-methyl-1-(propan-2-yl)-1H-pyrazol-4-yl]-7- |
| | (trifluoromethyl)[1,2,4]triazolo[1,5-c]quinazolin-5(6H)-one |
| | LC-MS (Method 1): Rₜ = 1.10 min; MS (ESIpos): m/z = 377 [M + H]⁺ |
| | ¹H-NMR (400 MHz, DMSO-d₆): δ [ppm] = 1.46 (d, 6H), 2.55 (s, 3H), 4.53 (spt, |
| | 1H), 7.57 (t, 1H), 8.07 (dd, 1H), 8.36 (s, 1H), 8.49 (dd, 1H), 11.53 (br s, 1H). |
| Intermediate 636 | 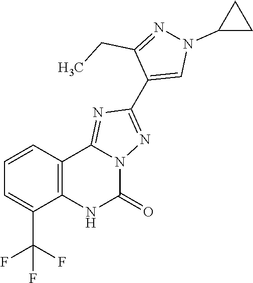 [SMILES: CCc1nn(C2CC2)cc1-c1nc2c3cccc(C(F)(F)F)c3[nH]c(=O)n2n1] |
| | 2-(1-cyclopropyl-3-ethyl-1H-pyrazol-4-yl)-7- |
| | (trifluoromethyl)[1,2,4]triazolo[1,5-c]quinazolin-5(6H)-one |
| | LC-MS (Method 1): Rₜ = 1.12 min; MS (ESIpos): m/z = 389 [M + H]⁺ |
| | ¹H-NMR (400 MHz, DMSO-d₆): δ [ppm] = 0.94-1.02 (m, 2H), 1.09-1.16 (m, |
| | 2H), 1.27 (t, 3H), 3.00 (q, 2H), 3.78 (tt, 1H), 7.57 (t, 1H), 8.07 (dd, 1H), 8.36 |
| | (s, 1H), 8.48 (dd, 1H), 11.53 (br s, 1H). |
| Intermediate 637 | 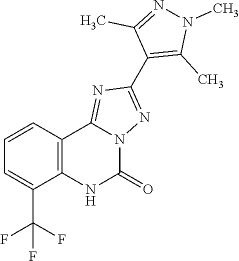 [SMILES: Cc1nn(C)c(C)c1-c1nc2c3cccc(C(F)(F)F)c3[nH]c(=O)n2n1] |
| | 7-(trifluoromethyl)-2-(1,3,5-trimethyl-1H-pyrazol-4-yl)[1,2,4]triazolo[1,5- |
| | c]quinazolin-5(6H)-one |
| | LC-MS (Method 1): Rₜ = 1.02 min; MS (ESIpos): m/z = 363 [M + H]⁺ |
| | ¹H-NMR (400 MHz, DMSO-d₆): δ [ppm] = 2.49 (s, 3H, in DMSO signal), 2.68 |
| | (s, 3H), 3.75 (s, 3H), 7.55 (t, 1H), 8.07 (dd, 1H), 8.52 (dd, 1H), 11.53 (br s, |
| | 1H). |

### Intermediate 638

### 7-methoxy-2-(1-methyl-1H-pyrazol-4-yl)[1,2,4]triazolo[1,5-c]quinazolin-5(6H)-one

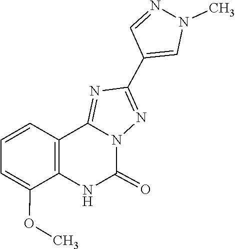 [SMILES: COc1cccc2c1[nH]c(=O)n1nc(-c3cnn(C)c3)nc21]

Di-tert-butyl (2-cyano-6-methoxyphenyl)-2-imidodicarbonate (400 mg, 1.15 mmol) and 1-methyl-1H-pyrazole-4-carbohydrazide (193 mg, 1.38 mmol) were stirred in DMF (3.7 mL) at 120° C. for 3 days. Then acetic acid (4 mL) was added at 100° C. and the reaction mixture was stirred for 24 h at this temperature. The reaction mixture was cooled to rt and added to water. After stirring for 10 minutes it was filtered, washed with water and the solid was dried to give 178 mg (29% yield, 56% purity) of the target compound, which was used without further purification.

LC-MS (Method 1): Rₜ=0.74 min; MS (ESIneg): m/z=295 [M−H]⁻.

The following compounds were prepared similarly to intermediate 638

| | Structure |
|---|---|
| | IUPAC-Name |
| | LC-MS (method): Retention time; Mass found |
| Example | ¹H-NMR |
| Intermediate 639 | 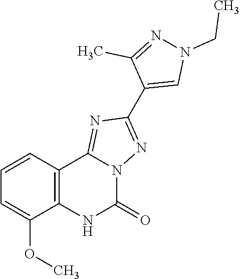 [SMILES: CCn1cc(-c2nc3c4cccc(OC)c4[nH]c(=O)n3n2)c(C)n1] |
| | 2-(1-ethyl-3-methyl-1H-pyrazol-4-yl)-7-methoxy[1,2,4]triazolo[1,5- |
| | c]quinazolin-5(6H)-one |
| | LC-MS (Method 1): Rₜ = 0.88 min; MS (ESIneg): m/z = 323 [M − H]⁻ |
| | ¹H-NMR (400 MHz, DMSO-d₆): δ [ppm] = 1.41 (t, 3H), 2.53 (s, 3H), 3.95 (s, |
| | 3H), 4.15 (q, 2H), 7.31-7.38 (m, 2H), 7.69-7.75 (m, 1H), 8.35 (s, 1H), |
| | 11.64 (s, 1H). |
| Intermediate 640 | 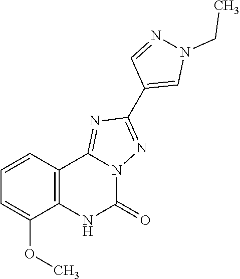 [SMILES: CCn1cc(-c2nc3c4cccc(OC)c4[nH]c(=O)n3n2)cn1] |
| | 2-(1-ethyl-1H-pyrazol-4-yl)-7-methoxy[1,2,4]triazolo[1,5-c]quinazolin- |
| | 5(6H)-one |
| | LC-MS (Method 1): Rₜ = 0.84 min; MS (ESIneg): m/z = 309 [M − H]⁻ |
| | ¹H-NMR (400 MHz, DMSO-d₆): δ [ppm] = 1.43 (t, 3H), 3.96 (s, 3H), 4.24 (q, |
| | 2H), 7.33-7.36 (m, 2H), 7.71-7.75 (m, 1H), 8.02 (d, 1H), 8.46 (d, 1H), |
| | 11.65 (s, 1H). |
| Intermediate 641 | 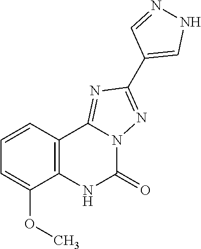 [SMILES: COc1cccc2c1[nH]c(=O)n1nc(-c3cn[nH]c3)nc21] |
| | 7-methoxy-2-(1H-pyrazol-4-yl)[1,2,4]triazolo[1,5-c]quinazolin-5(6H)-one |
| | LC-MS (Method 1): Rₜ = 0.70 min; MS (ESIneg): m/z = 281 [M − H]⁻ |
| | ¹H-NMR (400 MHz, DMSO-d₆): δ [ppm] = 3.96 (s, 3H), 7.32-7.39 (m, 2H), |
| | 7.71-7.78 (m, 1H), 8.08 (br s, 1H), 8.43 (br s, 1H), 11.65 (br s, 1H), 13.31 |
| | (br s, 1H). |
| Intermediate 642 | 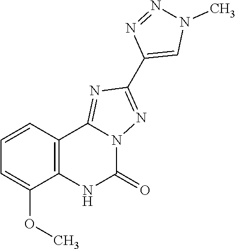 [SMILES: COc1cccc2c1[nH]c(=O)n1nc(-c3cn(C)nn3)nc21] |
| | 7-methoxy-2-(1-methyl-1H-1,2,3-triazol-4-yl)[1,2,4]triazolo[1,5- |
| | c]quinazolin-5(6H)-one |
| | LC-MS (Method 1): Rₜ = 0.70 min; MS (ESIneg): m/z = 296 [M − H]⁻ |
| | ¹H-NMR (400 MHz, DMSO-d₆): δ [ppm] = 3.97 (s, 3H), 4.16 (s, 3H), 7.35- |
| | 7.40 (m, 2H), 7.74-7.79 (m, 1H), 8.78 (s, 1H), 11.75 (s, 1H). |
| Intermediate 643 | 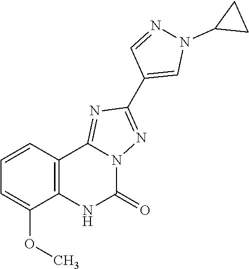 [SMILES: COc1cccc2c1[nH]c(=O)n1nc(-c3cnn(C4CC4)c3)nc21] |
| | 2-(1-cyclopropyl-1H-pyrazol-4-yl)-7-methoxy[1,2,4]triazolo[1,5- |
| | c]quinazolin-5(6H)-one |
| | LC-MS (Method 1): Rₜ = 0.88 min; MS (ESIneg): m/z = 321 [M − H]⁻ |
| | ¹H-NMR (400 MHz, DMSO-d₆): δ [ppm] = 0.98-1.04 (m, 2H), 1.13-1.18 (m, |
| | 2H), 3.87 (tt, 1H), 3.96 (s,3H), 7.33-7.38 (m, 2H), 7.70-7.75 (m, 1H), 8.01 |
| | (d, 1H), 8.48 (d, 1H), 11.66 (br s, 1H). |
| Intermediate 644 | 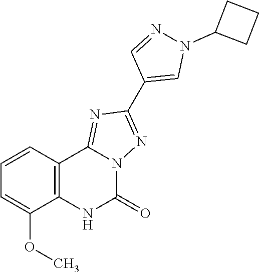 [SMILES: COc1cccc2c1[nH]c(=O)n1nc(-c3cnn(C4CCC4)c3)nc21] |
| | 2-(1-cyclobutyl-1H-pyrazol-4-yl)-7-methoxy[1,2,4]triazolo[1,5- |
| | c]quinazolin-5(6H)-one |
| | LC-MS (Method 1): Rₜ = 0.96 min; MS (ESIneg): m/z = 335 [M − H]⁻ |
| | ¹H-NMR (400 MHz, DMSO-d₆): δ [ppm] = 1.75-1.87 (m, 2H), 2.37-2.46 (m, |
| | 2H), 2.52-2.60 (m, 2H), 3.96 (s, 3H), 4.91-5.00 (m, 1H), 7.33-7.38 (m, |
| | 2H), 7.71-7.76 (m, 1H), 8.06 (d, 1H), 8.53 (d, 1H), 11.66 (br s, 1H). |
| Intermediate 645 | 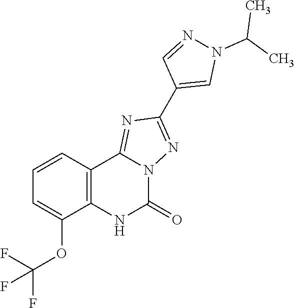 [SMILES: CC(C)n1cc(-c2nc3c4cccc(OC(F)(F)F)c4[nH]c(=O)n3n2)cn1] |
| | 2-[1-(propan-2-yl)-1H-pyrazol-4-yl]-7- |
| | (trifluoromethoxy)[1,2,4]triazolo[1,5-c]quinazolin-5(6H)-one |
| | LC-MS (Method 1): Rₜ = 1.05 min; MS (ESIneg): m/z = 377 [M − H]⁻ |
| | ¹H-NMR (400 MHz, DMSO-d₆): δ [ppm] = 1.49 (d, 6H), 4.63 (spt, 1H), 7.47 (t, |
| | 1H), 7.77 (ddq, 1H), 8.04 (s, 1H), 8.19 (dd, 1H), 8.48 (s, 1H), 12.51 (s, 1H). |

### Intermediate 646

### 2-(1-methyl-1H-pyrazol-4-yl)-5-oxo-5,6-dihydro[1,2,4]triazolo[1,5-c]quinazoline-7-carbonitrile

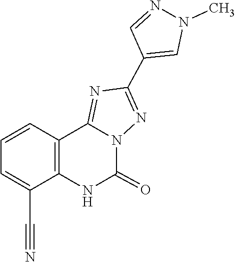 [SMILES: Cn1cc(-c2nc3c4cccc(C#N)c4[nH]c(=O)n3n2)cn1]

Di-tert-butyl (2,6-dicyanophenyl)-2-imidodicarbonate (212 mg, 617 μmol) and 1-methyl-1H-pyrazole-4-carbohydrazide (104 mg, 741 μmol) were stirred in DMF (3.7 mL) at 120° C. for 20 h. Then acetic acid (2 mL) was added at 100° C. and the reaction mixture was stirred for 24 h at this temperature. The reaction mixture was cooled to rt and added to water. After stirring for 10 minutes it was filtered, washed with water and the solid was dried under reduced pressure at 60° C. to give 120 mg (95% purity, 63% yield) of the target compound, which was used without further purification.

LC-MS (Method 1): Rₜ=0.71 min; MS (ESIpos): m/z=292 [M+H]⁺

¹H-NMR (400 MHz, DMSO-d₆): δ [ppm]=3.94 (s, 3H), 7.52 (t, 1H), 8.02 (d, 1H), 8.17 (dd, 1H), 8.43 (d, 1H), 8.46 (dd, 1H), 12.49 (br s, 1H).

The following intermediates were prepared analogously to intermediate 646

| | Structure |
|---|---|
| | IUPAC-Name |
| | LC-MS (method): Retention time; Mass found |
| Example | ¹H-NMR |
| Intermediate 647 | 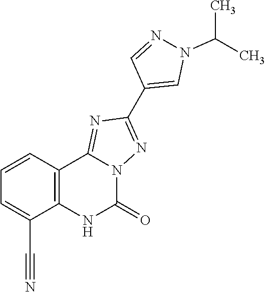 [SMILES: CC(C)n1cc(-c2nc3c4cccc(C#N)c4[nH]c(=O)n3n2)cn1] |
| | 5-oxo-2-[1-(propan-2-yl)-1H-pyrazol-4-yl]-5,6-dihydro[1,2,4]triazolo[1,5- |
| | c]quinazoline-7-carbonitrile |
| | LC-MS (Method 1): Rₜ = 0.87 min; MS (ESIpos): m/z = 320 [M + H]⁺ |
| | ¹H-NMR (400 MHz, DMSO-d₆): δ [ppm] = 1.48 (d, 6H), 4.62 (spt, 1H), 7.52 (t, |
| | 1H), 8.04 (d, 1H), 8.17 (dd, 1H), 8.45-8.50 (m, 2H), 12.49 (br s, 1H). |
| Intermediate 648 | 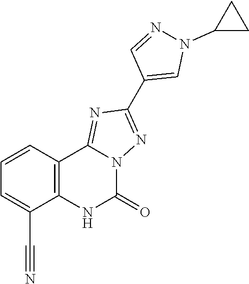 [SMILES: N#Cc1cccc2c1[nH]c(=O)n1nc(-c3cnn(C4CC4)c3)nc21] |
| | 2-(1-cyclopropyl-1H-pyrazol-4-yl)-5-oxo-5,6-dihydro[1,2,4]triazolo[1,5- |
| | c]quinazoline-7-carbonitrile |
| | LC-MS (Method 1): Rₜ = 0.82 min; MS (ESIpos): m/z = 318 [M + H]⁺ |
| | ¹H-NMR (400 MHz, DMSO-d₆): δ [ppm] = 0.99-1.05 (m, 2H), 1.13-1.19 (m, |
| | 2H), 3.87 (tt, 1H), 7.52 (t, 1H), 8.02 (d, 1H), 8.17 (dd, 1H), 8.46 (dd, 1H), |
| | 8.50 (d, 1H), 12.49 (br s, 1H). |
| Intermediate 649 | 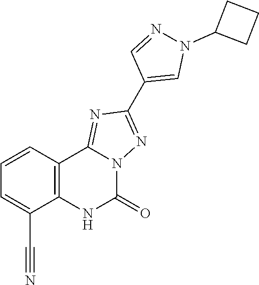 [SMILES: N#Cc1cccc2c1[nH]c(=O)n1nc(-c3cnn(C4CCC4)c3)nc21] |
| | 2-(1-cyclobutyl-1H-pyrazol-4-yl)-5-oxo-5,6-dihydro[1,2,4]triazolo[1,5- |
| | c]quinazoline-7-carbonitrile |
| | LC-MS (Method 1): Rₜ = 0.92 min; MS (ESIpos): m/z = 332 [M + H]⁺ |
| | ¹H-NMR (400 MHz, DMSO-d₆): δ [ppm] = 1.76-1.87 (m, 2H), 2.38-2.47 (m, |
| | 2H), 2.51-2.59 (m, 2H, partial in DMSO signal), 4.96 (br tt, 1H), 7.52 (t, |
| | 1H), 8.08 (d, 1H), 8.17 (dd, 1H), 8.47 (dd, 1H), 8.54 (d, 1H), 12.50 (br s, |
| | 1H). |
| Intermediate 650 | 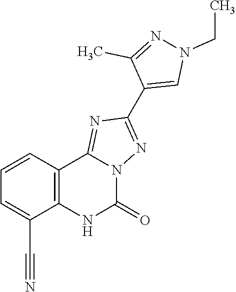 [SMILES: CCn1cc(-c2nc3c4cccc(C#N)c4[nH]c(=O)n3n2)c(C)n1] |
| | 2-(1-ethyl-3-methyl-1H-pyrazol-4-yl)-5-oxo-5,6- |
| | dihydro[1,2,4]triazolo[1,5-c]quinazoline-7-carbonitrile |
| | LC-MS (Method 1): Rₜ = 0.86 min; MS (ESIpos): m/z = 320 [M + H]⁺ |
| | ¹H-NMR (400 MHz, DMSO-d₆): δ [ppm] = 1.41 (t, 3H), 2.53 (s, 3H), 4.15 (q, |
| | 2H), 7.52 (t, 1H), 8.17 (dd, 1H), 8.36 (s, 1H), 8.45 (dd, 1H), 12.50 (br s, 1H). |

### Intermediate 651 and Intermediate 652

### 2-[1-(difluoromethyl)-1H-pyrazol-4-yl]-5-oxo-5,6-dihydro[1,2,4]triazolo[1,5-c]quinazoline-7-carbonitrile and 5-oxo-2-(1H-pyrazol-4-yl)-5,6-dihydro[1,2,4]triazolo[1,5-c]quinazoline-7-carbonitrile

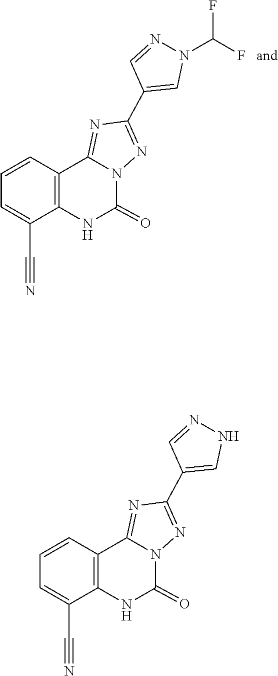 [SMILES: N#Cc1cccc2c1[nH]c(=O)n1nc(-c3cnn(C(F)F)c3)nc21 ; N#Cc1cccc2c1[nH]c(=O)n1nc(-c3cn[nH]c3)nc21]

Di-tert-butyl (2,6-dicyanophenyl)-2-imidodicarbonate (500 mg, 1.46 mmol) and a mixture of 1-methyl-1H-pyrazole-4-carbohydrazide and 1H-pyrazole-4-carbohydrazide (308 mg, −1.75 mmol) were stirred in DMF (8.7 mL) at 120° C. over night. Then acetic acid (9 mL) was added at 100° C. and the reaction mixture was stirred at this temperature over night. The reaction mixture was cooled to rt and added to water. After stirring for 10 minutes it was filtered, washed with water and the solid was dried to give 249 mg of about a 1:1 mixture of both target compounds, which were used without further purification.

LC-MS (Method 1):

Rₜ=0.81 min; MS (ESIneg): m/z=326 [M−H]⁻.

### 2-[1-(difluoromethyl)-1H-pyrazol-4-yl]-5-oxo-5,6-dihydro[1,2,4]triazolo[1,5-c]quinazoline-7-carbonitrile

Rₜ=0.62 min; MS (ESIneg): m/z=278 [M+H]+

### 5-oxo-2-(1H-pyrazol-4-yl)-5,6-dihydro[1,2,4]triazolo[1,5-c]quinazoline-7-carbonitrile

The following intermediates were prepared analogously to intermediate 646

| | Structure |
|---|---|
| | IUPAC-Name |
| | LC-MS (method): Retention time; Mass found |
| Example | ¹H-NMR |
| Intermediate 653 | 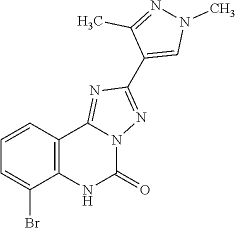 [SMILES: Cc1nn(C)cc1-c1nc2c3cccc(Br)c3[nH]c(=O)n2n1] |
| | 7-bromo-2-(1,3-dimethyl-1H-pyrazol-4-yl)[1,2,4]triazolo[1,5- |
| | c]quinazolin-5(6H)-one |
| | LC-MS (Method 2): Rₜ = 0.58 min; MS (ESIpos): m/z = 359 [M + H]⁺ |
| | ¹H-NMR (400 MHz, DMSO-d₆): δ [ppm] = 2.52-2.54 (m, 3H), 3.85 (s, 3H), |
| | 7.33 (t, 1H), 7.99 (dd, 1H), 8.19 (dd, 1H), 8.32 (s, 1H), 11.34 (s, 1H). |
| Intermediate 654 | 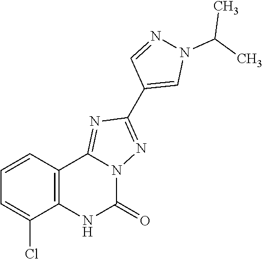 [SMILES: CC(C)n1cc(-c2nc3c4cccc(Cl)c4[nH]c(=O)n3n2)cn1] |
| | 7-chloro-2-(1-isopropyl-1H-pyrazol-4-yl)[1,2,4]triazolo[1,5-c]quinazolin- |
| | 5(6H)-oneLC-MS (Method 2): Rₜ = 0.63 min; MS (ESIpos): m/z = 329 |
| | [M + H]⁺ |
| | ¹H-NMR (400 MHz, DMSO-d₆): δ [ppm] = 1.48 (d, 6H), 4.62 (spt, 1H), 7.40 (t, |
| | 1H), 7.84 (dd, 1H), 8.04 (s, 1H), 8.16 (dd, 1H), 8.48 (s, 1H), 11.73 (s, 1H). |
| Intermediate 655 | 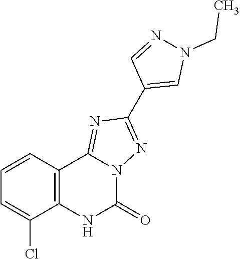 [SMILES: CCn1cc(-c2nc3c4cccc(Cl)c4[nH]c(=O)n3n2)cn1] |
| | 7-chloro-2-(1-ethyl-1H-pyrazol-4-yl)[1,2,4]triazolo[1,5-c]quinazolin- |
| | 5(6H)-one |
| | LC-MS (Method 2): Rₜ = 0.59 min; MS (ESIpos): m/z = 315 [M + H]⁺ |
| | ¹H-NMR (400 MHz, DMSO-d₆): δ [ppm] = 1.44 (t, 3H), 4.24 (q, 2H), 7.40 (t, |
| | 1H), 7.84 (dd, 1H), 8.03 (s, 1H), 8.16 (dd, 1H), 8.47 (s, 1H), 11.73 (s, 1H). |
| Intermediate 656 | 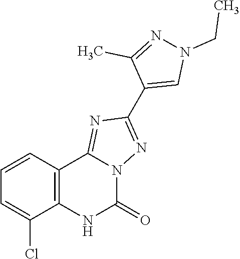 [SMILES: CCn1cc(-c2nc3c4cccc(Cl)c4[nH]c(=O)n3n2)c(C)n1] |
| | 7-chloro-2-(1-ethyl-3-methyl-1H-pyrazol-4-yl)[1,2,4]triazolo[1,5- |
| | c]quinazolin-5(6H)-one |
| | LC-MS (Method 2): Rₜ = 0.63 min; MS (ESIpos): m/z = 329 [M + H]⁺ |
| | ¹H-NMR (400 MHz, DMSO-d₆): δ [ppm] = 1.41 (t, 3H), 2.54 (s, 3H), 4.15 (q, |
| | 2H), 7.40 (t, 1H), 7.83 (dd, 1H), 8.14 (dd, 1H), 8.35 (s, 1H), 11.73 (s, 1H). |
| Intermediate 657 | 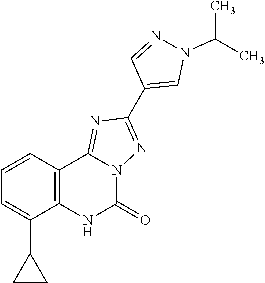 [SMILES: CC(C)n1cc(-c2nc3c4cccc(C5CC5)c4[nH]c(=O)n3n2)cn1] |
| | 7-cyclopropyl-2-[1-(propan-2-yl)-1H-pyrazol-4-yl][1,2,4]triazolo[1,5- |
| | c]quinazolin-5(6H)-one |
| | LC-MS (Method 2): Rₜ = 0.79 min; MS (ESIpos): m/z = 335 [M + H]⁺ |
| | ¹H-NMR (400 MHz, DMSO-d₆): δ [ppm] = 0.68-0.77 (m, 2H), 1.01-1.10 (m, |
| | 2H), 1.49 (d, 6H), 2.24-2.38 (m, 1H), 4.62 (spt, 1H), 7.25-7.35 (m, 1H), |
| | 7.37-7.42 (m, 1H), 7.99-8.08 (m, 2H), 8.47 (s, 1H), 11.48 (br s, 1H). |
| Intermediate 658 | 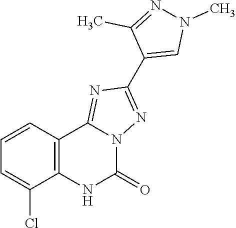 [SMILES: Cc1nn(C)cc1-c1nc2c3cccc(Cl)c3[nH]c(=O)n2n1] |
| | 7-chloro-2-(1,3-dimethyl-1H-pyrazol-4-yl)[1,2,4]triazolo[1,5- |
| | c]quinazolin-5(6H)-one |
| | LC-MS (Method 2): Rₜ = 0.58 min; MS (ESIpos): m/z = 315 [M + H]⁺ |
| | ¹H-NMR (400 MHz, DMSO-d₆): δ [ppm] = 2.53 (s, 3H), 3.85 (s, 3H), 7.40 (t, |
| | 1H), 7.83 (dd, 1H), 8.14 (dd, 1H), 8.31 (s, 1H), 11.72 (s, 1H). |
| Intermediate 659 | 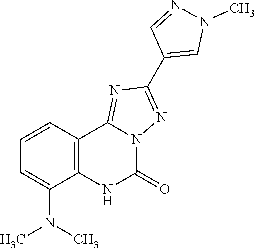 [SMILES: CN(C)c1cccc2c1[nH]c(=O)n1nc(-c3cnn(C)c3)nc21] |
| | 7-(dimethylamino)-2-(1-methyl-1H-pyrazol-4-yl)[1,2,4]triazolo[1,5- |
| | c]quinazolin-5(6H)-one |
| | LC-MS (Method 2): Rₜ = 0.63 min; MS (ESIpos): m/z = 310 [M + H]⁺ |
| | ¹H-NMR (400 MHz, DMSO-d₆): δ [ppm] = 2.71 (s, 6H), 3.94 (s, 3H), 7.30- |
| | 7.36 (m, 1H), 7.46 (dd, 1H), 7.86 (dd, 1H), 8.01 (s, 1H), 8.41 (s, 1H), 11.19 |
| | (br s, 1H). |
| Intermediate 660 | 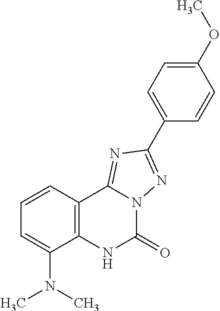 [SMILES: COc1ccc(-c2nc3c4cccc(N(C)C)c4[nH]c(=O)n3n2)cc1] |
| | 7-(dimethylamino)-2-(4-methoxyphenyl)[1,2,4]triazolo[1,5-c]quinazolin- |
| | 5(6H)-one |
| | LC-MS (Method 2): Rₜ = 0.89 min; MS (ESIpos): m/z = 336 [M + H]⁺ |
| | ¹H-NMR (400 MHz, DMSO-d₆): δ [ppm] = 2.71 (s, 6H), 3.85 (s, 3H), 7.10- |
| | 7.15 (m, 2H), 7.31-7.38 (m, 1H), 7.48 (dd, 1H), 7.92 (dd, 1H), 7.95 (s, 1H), |
| | 8.14-8.19 (m, 2H). |
| Intermediate 661 | 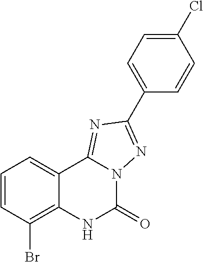 [SMILES: O=c1[nH]c2c(Br)cccc2c2nc(-c3ccc(Cl)cc3)nn12] |
| | 7-bromo-2-(4-chlorophenyl)[1,2,4]triazolo[1,5-c]quinazolin-5(6H)-one |
| | LC-MS (Method 2): Rₜ = 0.75 min; MS (ESIpos): m/z = 375 [M + H]⁺ |
| | ¹H-NMR (400 MHz, DMSO-d₆): δ [ppm] = 7.35 (t, 1H), 7.64-7.67 (m, 2H), |
| | 8.01 (dd, 1H), 8.21-8.25 (m, 2H), 8.26 (dd, 1H), 11.47 (br s, 1H). |
| Intermediate 662 | 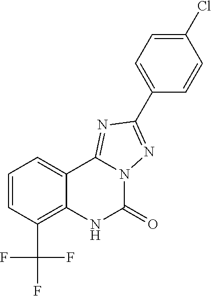 [SMILES: O=c1[nH]c2c(C(F)(F)F)cccc2c2nc(-c3ccc(Cl)cc3)nn12] |
| | 2-(4-chlorophenyl)-7-(trifluoromethyl)[1,2,4]triazolo[1,5-c]quinazolin- |
| | 5(6H)-one |
| | LC-MS (Method 2): Rₜ = 0.77 min; MS (ESIpos): m/z = 365 [M + H]⁺ |
| | ¹H-NMR (400 MHz, DMSO-d₆): δ [ppm] = 7.58 (t, 1H), 7.64-7.68 (m, 2H), |
| | 8.09 (d, 1H), 8.22-8.26 (m, 2H), 8.55 (d, 1H), 11.58-11.76 (m, 1H). |
| Intermediate 663 | 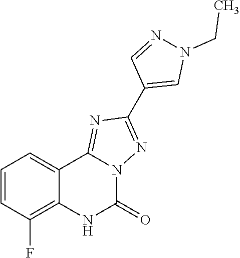 [SMILES: CCn1cc(-c2nc3c4cccc(F)c4[nH]c(=O)n3n2)cn1] |
| | 2-(1-ethyl-1H-pyrazol-4-yl)-7-fluoro[1,2,4]triazolo[1,5-c]quinazolin- |
| | 5(6H)-one |
| | LC-MS (Method 2): Rₜ = 0.54 min; MS (ESIpos): m/z = 299 [M + H]⁺ |
| | ¹H-NMR (400 MHz, DMSO-d₆): δ [ppm] = 1.44 (t, 3H), 4.24 (q, 2H), 7.38 (td, |
| | 1H), 7.62 (ddd, 1H), 7.98 (d, 1H), 8.03 (s, 1H), 8.47 (s, 1H), 12.38 (br s, 1H). |
| Intermediate 664 | 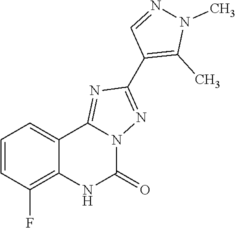 [SMILES: Cc1c(-c2nc3c4cccc(F)c4[nH]c(=O)n3n2)cnn1C] |
| | 2-(1,5-dimethyl-1H-pyrazol-4-yl)-7-fluoro[1,2,4]triazolo[1,5-c]quinazolin- |
| | 5(6H)-one |
| | LC-MS (Method 2): Rₜ = 0.54 min; MS (ESIpos): m/z = 299 [M + H]⁺ |
| | ¹H-NMR (400 MHz, DMSO-d₆): δ [ppm] = 2.71 (s, 3H), 3.83 (s, 3H), 7.36 (td, |
| | 1H), 7.57-7.64 (m, 1H), 7.94 (s, 1H), 8.01 (d, 1H), 12.39 (br s, 1H). |
| Intermediate 665 | 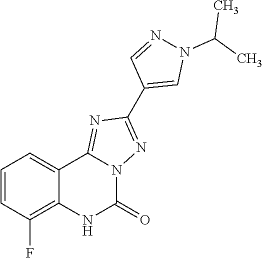 [SMILES: CC(C)n1cc(-c2nc3c4cccc(F)c4[nH]c(=O)n3n2)cn1] |
| | 7-fluoro-2-[1-(propan-2-yl)-1H-pyrazol-4-yl][1,2,4]triazolo[1,5- |
| | c]quinazolin-5(6H)-one |
| | LC-MS (Method 2): Rₜ = 0.58 min; MS (ESIpos): m/z = 313 [M + H]⁺ |
| | ¹H-NMR (400 MHz, DMSO-d₆): δ [ppm] = 1.48 (d, 6H), 4.62 (spt, 1H), 7.38 |
| | (td, 1H), 7.62 (ddd, 1H), 7.99 (d, 1H), 8.04 (s, 1H), 8.48 (s, 1H), 12.38 (br s, |
| | 1H). ). |

### Intermediate 666

### 2-(4-methoxyphenyl)-7-(methylsulfanyl)[1,2,4]triazolo[1,5-c]quinazolin-5(6H)-one

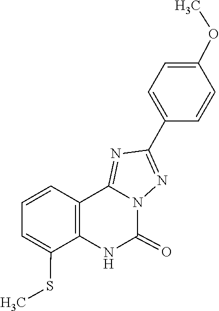 [SMILES: COc1ccc(-c2nc3c4cccc(SC)c4[nH]c(=O)n3n2)cc1]

Ethyl [2-cyano-6-(methylsulfanyl)phenyl]carbamate (136 mg, 574 μmol) and 4-methoxybenzohydrazide (115 mg, 689 μmol) were stirred in DMF (3.0 mL) at 120° C. overnight. trifluoroacetic acid (220 μL, 2.9 mmol) was added to the mixture and it was stirred for 2 h at 90° C. The reaction was cooled to rt and water was added to the mixture. The solid was filtered, washed with water and dried under reduced pressure at 60° C. to give 174 mg (95% purity, 85% yield) of the title compound.

LC-MS (method 2): Rₜ=0.64 min; MS (ESIneg): m/z=337 [M−H]⁻.

¹H-NMR (400 MHz, DMSO-d6): δ [ppm]=2.54 (s, 3H), 3.85 (s, 3H), 7.09-7.16 (m, 2H), 7.42 (t, 1H), 7.80 (dd, 1H), 8.12-8.20 (m, 3H), 11.12 (br s, 1H).

The following intermediates were prepared similarly to intermediate 666 using acetic acid instead of TFA

| | Structure |
|---|---|
| | IUPAC-Name |
| | LC-MS (method): Retention time; Mass found |
| Example | ¹H-NMR |
| Intermediate 667 | 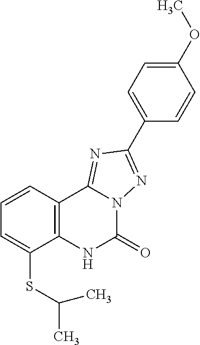 [SMILES: COc1ccc(-c2nc3c4cccc(SC(C)C)c4[nH]c(=O)n3n2)cc1] |
| | 2-(4-methoxyphenyl)-7-[(propan-2-yl)sulfanyl][1,2,4]triazolo[1,5- |
| | c]quinazolin-5(6H)-one |
| | LC-MS (Method 2): Rₜ = 0.89 min; MS (ESIpos): m/z = 367 [M + H]⁺ |
| | ¹H-NMR (400 MHz, DMSO-d₆): δ [ppm] = 1.23 (d, 6H), 3.42 (spt, 1H), |
| | 3.85 (s, 3H), 7.10-7.15 (m, 2H), 7.40 (t, 1H), 7.86 (br d, 1H), 8.15-8.19 |
| | (m, 2H), 8.24 (dd, 1H), 11.00 (br s, 1H). |
| Intermediate 668 | 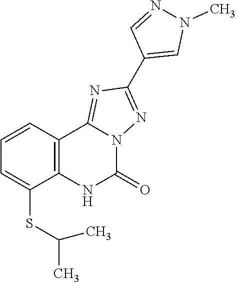 [SMILES: CC(C)Sc1cccc2c1[nH]c(=O)n1nc(-c3cnn(C)c3)nc21] |
| | 2-(1-methyl-1H-pyrazol-4-yl)-7-[(propan-2-yl)sulfanyl][1,2,4]triazolo[1,5- |
| | c]quinazolin-5(6H)-one |
| | LC-MS (Method 2): Rₜ = 0.67 min; MS (ESIpos): m/z = 341 [M + H]⁺ |
| | ¹H-NMR (400 MHz, DMSO-d₆): δ [ppm] = 1.22 (d, 6H), 3.36-3.44 (m, 1H), |
| | 3.94 (s, 3H), 7.40 (t, 1H), 7.87 (dd, 1H), 8.02 (d, 1H), 8.20 (dd, 1H), 8.42 (s, |
| | 1H), 10.96 (br s, 1H). |
| Intermediate 669 | 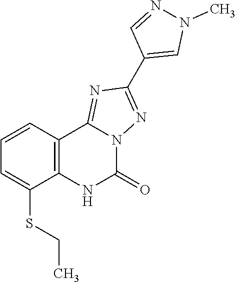 [SMILES: CCSc1cccc2c1[nH]c(=O)n1nc(-c3cnn(C)c3)nc21] |
| | 7-(ethylsulfanyl)-2-(1-methyl-1H-pyrazol-4-yl)[1,2,4]triazolo[1,5- |
| | c]quinazolin-5(6H)-one |
| | LC-MS (Method 2): Rₜ = 0.59 min; MS (ESIpos): m/z = 327 [M + H]⁺ |
| | ¹H-NMR (400 MHz, DMSO-d₆): δ [ppm] = 1.20 (t, 3H), 2.95 (q, 2H), |
| | 3.94 (s, 3H), 7.39 (t, 1H), 7.76-7.88 (m, 1H), 8.02 (d, 1H), 8.14 (dd, 1H), |
| | 8.42 (s, 1H), 10.86- 11.09 (m, 1H). |
| Intermediate 670 | 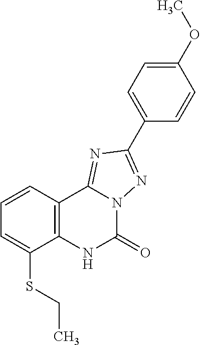 [SMILES: CCSc1cccc2c1[nH]c(=O)n1nc(-c3ccc(OC)cc3)nc21] |
| | 7-(ethylsulfanyl)-2-(4-methoxyphenyl)[1,2,4]triazolo[1,5-c]quinazolin- |
| | 5(6H)-one |
| | LC-MS (Method 2): Rₜ = 0.75 min; MS (ESIpos): m/z = 353 [M + H]⁺ |
| | ¹H-NMR (400 MHz, DMSO-d₆): δ [ppm] = 1.20 (t, 3H), 2.96 (q, 2H), |
| | 3.85 (s, 3H), 7.11-7.15 (m, 2H), 7.41 (t, 1H), 7.86 (dd, 1H), 8.15-8.18 |
| | (m, 2H), 8.20 (dd, 1H), 11.03 (br s, 1H). |
| Intermediate 671 | 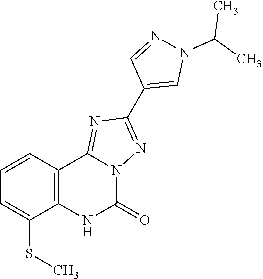 [SMILES: CSc1cccc2c1[nH]c(=O)n1nc(-c3cnn(C(C)C)c3)nc21] |
| | 7-(methylsulfanyl)-2-[1-(propan-2-yl)-1H-pyrazol-4-yl][1,2,4]triazolo[1,5- |
| | c]quinazolin-5(6H)-one |
| | LC-MS (Method 2): Rₜ = 0.62 min; MS (ESIpos): m/z = 341 [M + H]⁺ |
| | ¹H-NMR (400 MHz, DMSO-d₆): δ [ppm] = 1.49 (d, 6H), 2.53 (s, 3H), |
| | 4.63 (spt, 1H), 7.41 (t, 1H), 7.79 (dd, 1H), 8.04 (s, 1H), 8.10 (dd, 1H), |
| | 8.48 (s, 1H),11.08 (s, 1H). |
| Intermediate 672 | 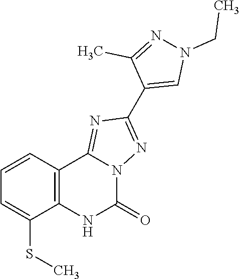 [SMILES: CCn1cc(-c2nc3c4cccc(SC)c4[nH]c(=O)n3n2)c(C)n1] |
| | 2-(1-ethyl-3-methyl-1H-pyrazol-4-yl)-7- |
| | (methylsulfanyl)[1,2,4]triazolo[1,5-c]quinazolin-5(6H)-one |
| | LC-MS (Method 2): Rₜ = 0.62 min; MS (ESIpos): m/z = 341 [M + H]⁺ |
| | ¹H-NMR (400 MHz, DMSO-d₆): δ [ppm] = 1.41 (t, 3H), 2.53 (s, 3H), |
| | 2.54 (s, 3H), 4.15 (q, 2H), 7.41 (t, 1H), 7.78 (dd, 1H), 8.08 (dd, 1H), |
| | 8.35 (s, 1H), 11.07 (br s, 1H). |

### Intermediate 673

### 7-bromo-2-(1-methyl-1H-pyrazol-5-yl)[1,2,4]triazolo[1,5-c]quinazolin-5(6H)-one

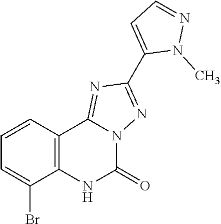 [SMILES: Cn1nccc1-c1nc2c3cccc(Br)c3[nH]c(=O)n2n1]

Ethyl (2-bromo-6-cyanophenyl)carbamate (500 mg, 1.86 mmol) and 1-methyl-1H-pyrazole-5-carbohydrazide (312 mg, 2.23 mmol) were stirred in DMF (7.7 mL) for 24 h at 120° C. The reaction mixture was allowed to reach rt and poured into water (50 mL). The precipitate was filtered off, washed four times with water and dried under reduced pressure at 50° C. to give 560 mg of a solid material. Trifluoroacetc acid (720 μL, 9.3 mmol) was added to the solid material in dichloroethane (13 mL). It was stirred for 4 h at 90° C. The reaction mixture was allowed to cool down and concentrated. The residue was dried under reduced pressure at 45° C. to yield 580 mg (90%) of the title compound, which was used without further purification in the next step.

LC-MS (Method 2): Rₜ=0.65 min; MS (ESIpos): m/z=345 [M+H]⁺

¹H-NMR (400 MHz, DMSO-d₆): δ [ppm]=4.30 (s, 3H), 7.00 (d, 1H), 7.36 (t, 1H), 7.61 (d, 1H), 8.03 (dd, 1H), 8.27 (dd, 1H), 11.54 (br s, 1H).

The following intermediates were prepared analogously to intermediate 673

| | Structure |
|---|---|
| | IUPAC-Name |
| | LC-MS (method): Retention time; Mass found |
| Example | ¹H-NMR |
| Intermediate 674 | 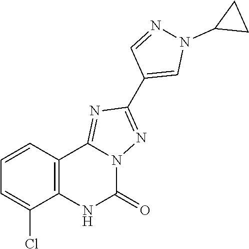 [SMILES: O=c1[nH]c2c(Cl)cccc2c2nc(-c3cnn(C4CC4)c3)nn12] |
| | 7-chloro-2-(1-cyclopropyl-1H-pyrazol-4-yl)[1,2,4]triazolo[1,5- |
| | c]quinazolin-5(6H)-one |
| | LC-MS (Method 2): Rₜ = 0.56 min; MS (ESIneg): m/z = 325 [M − H]⁻ |
| | ¹H-NMR (400 MHz, DMSO-d₆): δ [ppm] = 0.98-1.06 (m, 2H), 1.12-1.21 (m, |
| | 2H), 3.87 (tt, 1H), 7.40 (t, 1H), 7.84 (dd, 1H), 8.00-8.03 (m, 1H), 8.15 (dd, |
| | 1H), 8.49 (s, 1H), 11.73 (s, 1H). |
| Intermediate 675 | 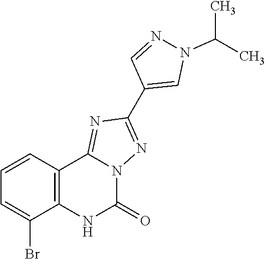 [SMILES: CC(C)n1cc(-c2nc3c4cccc(Br)c4[nH]c(=O)n3n2)cn1] |
| | 7-bromo-2-(1-isopropyl-1H-pyrazol-4-yl)[1,2,4]triazolo[1,5-c]quinazolin- |
| | 5(6H)-oneLC-MS (Method 2): Rₜ = 0.61 min; MS (ESIpos): m/z = 373 |
| | [M + H]⁺ |
| | ¹H-NMR (400 MHz, DMSO-d₆): δ [ppm] = 1.48 (d, 6H), 4.62 (spt, 1H), 7.34 (t, |
| | 1H), 8.00 (dd, 1H), 8.04 (d, 1H), 8.20 (dd, 1H), 8.48 (s, 1H), 11.35 (s, 1H). |
| Intermediate 676 | 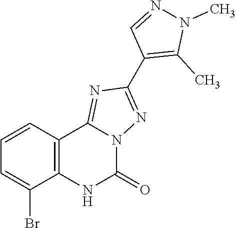 [SMILES: Cc1c(-c2nc3c4cccc(Br)c4[nH]c(=O)n3n2)cnn1C] |
| | 7-bromo-2-(1,5-dimethyl-1H-pyrazol-4-yl)[1,2,4]triazolo[1,5- |
| | c]quinazolin-5(6H)-one |
| Intermediate 677 | 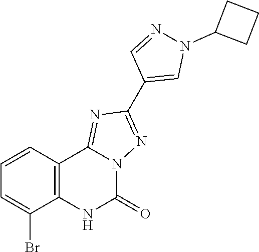 [SMILES: O=c1[nH]c2c(Br)cccc2c2nc(-c3cnn(C4CCC4)c3)nn12] |
| | 7-bromo-2-(1-cyclobutyl-1H-pyrazol-4-yl)[1,2,4]triazolo[1,5- |
| | c]quinazolin-5(6H)-one |
| | LC-MS (Method 2): Rt = 0.62 min; MS (ESIpos): miz = 385 [M + H]⁺ |
| Intermediate 678 | 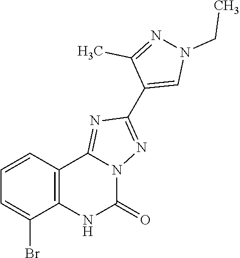 [SMILES: CCn1cc(-c2nc3c4cccc(Br)c4[nH]c(=O)n3n2)c(C)n1] |
| | 7-bromo-2-(1-ethyl-3-methyl-1H-pyrazol-4-yl)[1,2,4]triazolo[1,5- |
| | c]quinazolin-5(6H)-one |
| | LC-MS (Method 2): Rt = 0.63 min; MS (ESIpos): m/z = 373 [M + H]⁺ |
| Intermediate 679 | 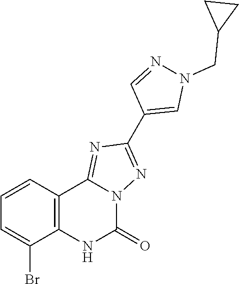 [SMILES: O=c1[nH]c2c(Br)cccc2c2nc(-c3cnn(CC4CC4)c3)nn12] |
| | 7-bromo-2-[1-(cyclopropylmethyl)-1H-pyrazol-4-yl][1,2,4]triazolo[1,5- |
| | c]quinazolin-5(6H)-one |
| | LC-MS (Method 2): Rt = 0.59 min; MS (ESIpos): m/z = 385 [M + H]⁺ |
| Intermediate 680 | 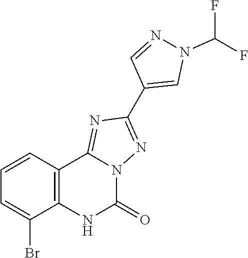 [SMILES: O=c1[nH]c2c(Br)cccc2c2nc(-c3cnn(C(F)F)c3)nn12] |
| | 7-bromo-2-[1-(difluoromethyl)-1H-pyrazol-4-yl][1,2,4]triazolo[1,5- |
| | c]quinazolin-5(6H)-one |
| | LC-MS (Method 2): Rt = 0.59 min; MS (ESIpos): m/z = 381 [M + H]⁺ |
| Intermediate 681 | 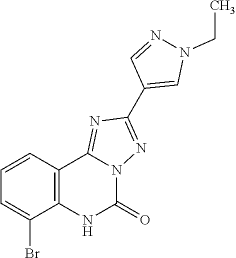 [SMILES: CCn1cc(-c2nc3c4cccc(Br)c4[nH]c(=O)n3n2)cn1] |
| | 7-bromo-2-(1-ethyl-1H-pyrazol-4-yl)[1,2,4]triazolo[1,5-c]quinazolin- |
| | 5(6H)-one |
| | LC-MS (Method 2): Rt = 0.57 min; MS (ESIpos): m/z = 359 [M + H]⁺ |
| Intermediate 682 | 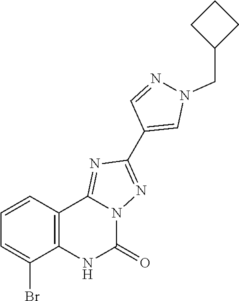 [SMILES: O=c1[nH]c2c(Br)cccc2c2nc(-c3cnn(CC4CCC4)c3)nn12] |
| | 7-bromo-2-[1-(cyclobutylmethyl)-1H-pyrazol-4-yl][1,2,4]triazolo[1,5- |
| | c]quinazolin-5(6H)-one |
| | LC-MS (Method 2): Rt = 0.64 min; MS (ESIpos): m/z = 399 [M + H]⁺ |

### Intermediate 683

### 5-chloro-2-(1-methyl-1H-pyrazol-4-yl)-7-(trifluoromethyl)[1,2,4]triazolo[1,5-c]quinazoline

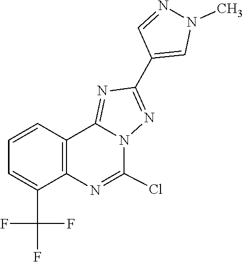 [SMILES: Cn1cc(-c2nc3c4cccc(C(F)(F)F)c4nc(Cl)n3n2)cn1]

To a suspension of 2-(1-methyl-1H-pyrazol-4-yl)-7-(trifluoromethyl)[1,2,4]triazolo[1,5-c]quinazolin-5(6H)-one (2.00 g, 5.98 mmol) in phosphorus oxychloride (17 mL, 180 mmol) N,N-diisopropylethylamine (21 mL, 120 mmol) was added carefully and the mixture was stirred for 2 h at 120° C. The mixture was cooled to rt and poured into ice, stirred for 1 h, filtered, washed with water and dried to give 1.99 g (100% purity, 94% yield) of the target compound, which was used without further purification.

LC-MS (Method 1): Rₜ=1.18 min; MS (ESIpos): m/z=353 [M+H]⁺

¹H-NMR (400 MHz, DMSO-d₆): δ [ppm]=3.96 (s, 3H), 7.97 (t, 1H), 8.11 (s, 1H), 8.34 (d, 1H), 8.56 (s, 1H), 8.74 (d, 1H).

The following intermediates were prepared analogously to intermediate 683

| | Structure |
|---|---|
| | IUPAC-Name |
| | LC-MS (method): Retention time; Mass found |
| Example | ¹H-NMR |
| Intermediate 684 | 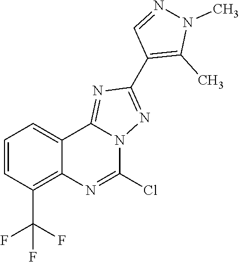 [SMILES: Cc1c(-c2nc3c4cccc(C(F)(F)F)c4nc(Cl)n3n2)cnn1C] |
| | 5-chloro-2-(1,5-dimethyl-1H-pyrazol-4-yl)-7- |
| | (trifluoromethyl)[1,2,4]triazolo[1,5-c]quinazoline |
| | LC-MS (Method 1): Rₜ = 1.25 min; MS (ESIpos): m/z = 367 [M + H]⁺ |
| | ¹H-NMR (400 MHz, DMSO-d₆): δ [ppm] = 2.76 (s, 3H), 3.85 (s, 3H), |
| | 7.96 (t, 1H), 8.01 (s, 1H), 8.34 (d, 1H), 8.75 (d, 1H). |
| Intermediate 685 | 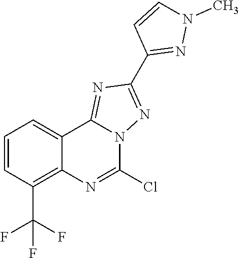 [SMILES: Cn1ccc(-c2nc3c4cccc(C(F)(F)F)c4nc(Cl)n3n2)n1] |
| | 5-chloro-2-(1-methyl-1H-pyrazol-3-yl)-7- |
| | (trifluoromethyl)[1,2,4]triazolo[1,5-c]quinazoline |
| | LC-MS (Method 1): Rₜ = 1.15 min; MS (ESIpos): m/z = 353 [M + H]⁺ |
| | ¹H-NMR (400 MHz, DMSO-d₆): δ [ppm] = 4.00 (s, 3H), 6.96 (d, 1H), |
| | 7.92 (d, 1H), 7.99 (t, 1H), 8.35 (dd, 1H), 8.79 (dd, 1H). |
| Intermediate 686 | 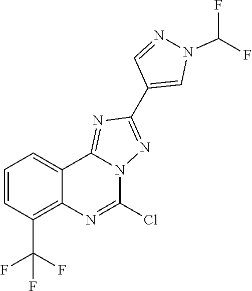 [SMILES: FC(F)n1cc(-c2nc3c4cccc(C(F)(F)F)c4nc(Cl)n3n2)cn1] |
| | 5-chloro-2-[1-(difluoromethyl)-1H-pyrazol-4-yl]-7- |
| | (trifluoromethyl)[1,2,4]triazolo[1,5-c]quinazoline |
| | LC-MS (Method 1): Rₜ = 1.30 min; MS (ESIpos): m/z = 389 [M + H]⁺ |
| | ¹H-NMR (400 MHz, DMSO-d₆): δ [ppm] = 7.94 (t, 1H), 8.00 (t, 1H), |
| | 8.37 (dd, 1H), 8.49 (s, 1H), 8.77 (dd, 1H), 9.09 (s, 1H). |
| Intermediate 687 | 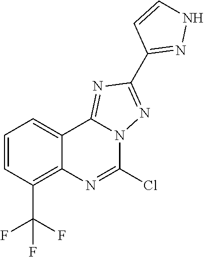 [SMILES: FC(F)(F)c1cccc2c1nc(Cl)n1nc(-c3cc[nH]n3)nc21] |
| | 5-chloro-2-(1H-pyrazol-3-yl)-7-(trifluoromethyl)[1,2,4]triazolo[1,5- |
| | c]quinazoline |
| | LC-MS (Method 1): Rₜ = 1.11 min; MS (ESIpos): m/z = 339 [M + H]⁺ |
| | ¹H-NMR (400 MHz, DMSO-d₆): δ [ppm] = 7.00 (d, 1H), 7.93 (d, 1H), |
| | 8.00 (t, 1H), 8.36 (dd, 1H), 8.79 (dd, 1H), 10.02 (br s, 1H). |
| Intermediate 688 | 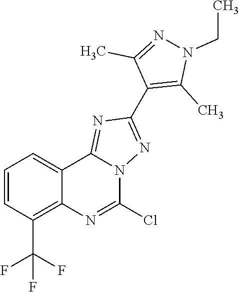 [SMILES: CCn1nc(C)c(-c2nc3c4cccc(C(F)(F)F)c4nc(Cl)n3n2)c1C] |
| | 5-chloro-2-(1-ethyl-3,5-dimethyl-1H-pyrazol-4-yl)-7- |
| | (trifluoromethyl)[1,2,4]triazolo[1,5-c]quinazoline |
| | LC-MS (Method 1): Rₜ = 1.43 min; MS (ESIpos): m/z = 395 [M + H]⁺ |
| | ¹H-NMR (400 MHz, DMSO-d₆): δ [ppm] = 1.34 (t, 3H), 2.55 (s, 3H), |
| | 2.74 (s, 3H), 4.11 (q, 2H), 7.96 (t, 1H), 8.34 (dd, 1H), 8.75 (dd, 1H). |
| Intermediate 689 | 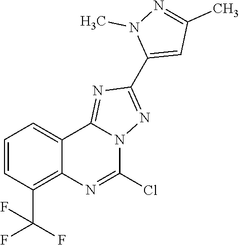 [SMILES: Cc1cc(-c2nc3c4cccc(C(F)(F)F)c4nc(Cl)n3n2)n(C)n1] |
| | 5-chloro-2-(1,3-dimethyl-1H-pyrazol-5-yl)-7- |
| | (trifluoromethyl)[1,2,4]triazolo[1,5-c]quinazoline |
| | LC-MS (Method 1): Rₜ = 1.34 min; MS (ESIpos): m/z = 367 [M + H]⁺ |
| | ¹H-NMR (500 MHz, DMSO-d₆): δ [ppm] = 2.25 (s, 3H), 4.27 (s, 3H), |
| | 6.89 (s, 1H), 8.01 (t, 1H), 8.39 (dd, 1H), 8.80 (dd, 1H). |
| Intermediate 690 | 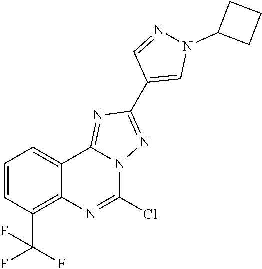 [SMILES: FC(F)(F)c1cccc2c1nc(Cl)n1nc(-c3cnn(C4CCC4)c3)nc21] |
| | 5-chloro-2-(1-cyclobutyl-1H-pyrazol-4-yl)-7- |
| | (trifluoromethyl)[1,2,4]triazolo[1,5-c]quinazoline |
| | LC-MS (Method 1): Rₜ = 1.34 min; MS (ESIpos): m/z = 393 [M + H]⁺ |
| | ¹H-NMR (400 MHz, DMSO-d₆): δ [ppm] = 1.76-1.89 (m, 2H), 2.39-2.47 |
| | (m, 2H), 2.53-2.61 (m, 2H), 4.98 (tt, 1H), 7.97 (t, 1H), 8.16 (d, 1H), |
| | 8.34 (dd ,1H), 8.66 (d, 1H), 8.74 (dd, 1H). |
| Intermediate 691 | 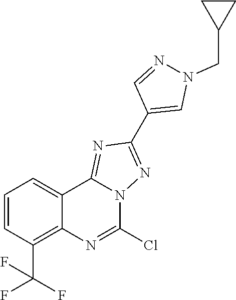 [SMILES: FC(F)(F)c1cccc2c1nc(Cl)n1nc(-c3cnn(CC4CC4)c3)nc21] |
| | 5-chloro-2-[1-(cyclopropylmethyl)-1H-pyrazol-4-yl]-7- |
| | (trifluoromethyl)[1,2,4]triazolo[1,5-c]quinazoline |
| | LC-MS (Method 1): Rₜ = 1.30 min; MS (ESIpos): m/z = 393 [M + H]⁺ |
| | ¹H-NMR (400 MHz, DMSO-d₆): δ [ppm] = 0.40-0.47 (m, 2H), 0.53-0.60 |
| | (m, 2H), 1.29-1.39 (m, 1H), 4.09 (d, 2H), 7.97 (t, 1H), 8.13 (s, 1H), |
| | 8.35 (dd, 1H), 8.60 (s, 1H), 8.75 (dd, 1H). |
| Intermediate 692 | 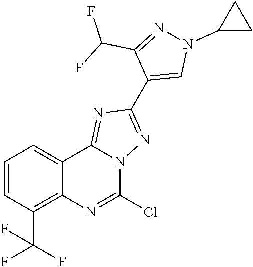 [SMILES: FC(F)c1nn(C2CC2)cc1-c1nc2c3cccc(C(F)(F)F)c3nc(Cl)n2n1] |
| | 5-chloro-2-[1-cyclopropyl-3-(difluoromethyl)-1H-pyrazol-4-yl]-7- |
| | (trifluoromethyl)[1,2,4]triazolo[1,5-c]quinazoline |
| | LC-MS (Method 1): Rₜ = 1.37 min; MS (ESIpos): m/z = 429 [M + H]⁺ |
| | ¹H-NMR (400 MHz, DMSO-d₆): δ [ppm] = 1.03-1.11 (m, 2H), 1.20-1.29 |
| | (m, 2H), 3.98 (tt, 1H), 7.63 (t, 1H), 8.00 (t, 1H), 8.37 (dd, 1H), 8.75-8.80 |
| | (m, 2H). |
| Intermediate 693 | 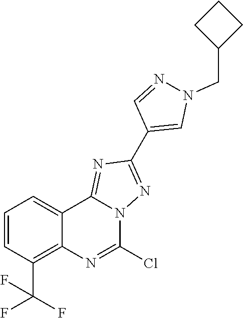 [SMILES: FC(F)(F)c1cccc2c1nc(Cl)n1nc(-c3cnn(CC4CCC4)c3)nc21] |
| | 5-chloro-2-[1-(cyclobutylmethyl)-1H-pyrazol-4-yl]-7- |
| | (trifluoromethyl)[1,2,4]triazolo[1,5-c]quinazoline |
| | LC-MS (Method 1): Rₜ = 1.40 min; MS (ESIpos): m/z = 407 [M + H]⁺ |
| | ¹H-NMR (400 MHz, DMSO-d₆): δ [ppm] = 1.76-2.04 (m, 6H), 2.78-2.89 |
| | (m, 1H), 4.26 (d, 2H), 7.97 (t, 1H), 8.12 (d, 1H), 8.35 (dd, 1H), 8.57 |
| | (d, 1H), 8.74 (dd, 1H). |
| Intermediate 694 | 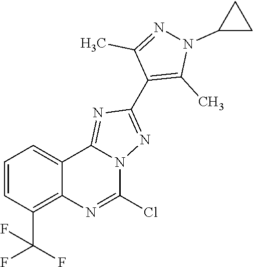 [SMILES: Cc1nn(C2CC2)c(C)c1-c1nc2c3cccc(C(F)(F)F)c3nc(Cl)n2n1] |
| | 5-chloro-2-(1-cyclopropyl-3,5-dimethyl-1H-pyrazol-4-yl)-7- |
| | (trifluoromethyl)[1,2,4]triazolo[1,5-c]quinazoline |
| | LC-MS (Method 1): Rₜ = 1.47 min; MS (ESIpos): m/z = 407 [M + H]⁺ |
| | ¹H-NMR (400 MHz, DMSO-d₆): δ [ppm] = 1.01-1.12 (m, 4H), 2.52 |
| | (s, 3H), 2.81 (s, 3H), 3.52-3.58 (m, 1H), 7.96 (t, 1H), 8.33 (dd, 1H), |
| | 8.74 (dd, 1H). |
| Intermediate 695 | 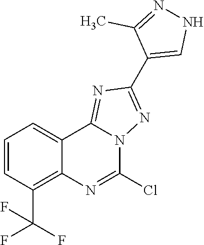 [SMILES: Cc1n[nH]cc1-c1nc2c3cccc(C(F)(F)F)c3nc(Cl)n2n1] |
| | 5-chloro-2-(3-methyl-1H-pyrazol-4-yl)-7- |
| | (trifluoromethyl)[1,2,4]triazolo[1,5-c]quinazoline |
| | LC-MS (Method 1): Rₜ = 1.19 min; MS (ESIpos): m/z = 353 [M + H]⁺ |
| | ¹H-NMR (400 MHz, DMSO-d₆): δ [ppm] = 2.67 (s, 3H), 7.96 (t, 1H), |
| | 8.19 (s, 1H), 8.33 (dd, 1H), 8.74 (dd, 1H). |
| Intermediate 696 | 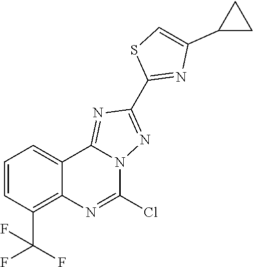 [SMILES: FC(F)(F)c1cccc2c1nc(Cl)n1nc(-c3nc(C4CC4)cs3)nc21] |
| | 5-chloro-2-(4-cyclopropyl-1,3-thiazol-2-yl)-7- |
| | (trifluoromethyl)[1,2,4]triazolo[1,5-c]quinazoline |
| | LC-MS (Method 1): Rₜ = 1.43 min; MS (ESIpos): m/z = 396 [M + H]⁺ |
| | ¹H-NMR (400 MHz, DMSO-d₆): δ [ppm] = 0.92-1.05 (m, 4H), 2.21-2.29 (m, |
| | 1H), 7.68 (s, 1H), 8.01 (t, 1H), 8.39 (dd, 1H), 8.83 (dd, 1H). |
| Intermediate 697 | 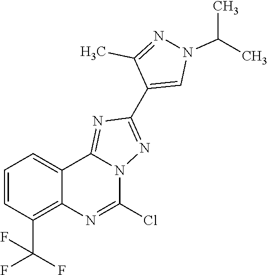 [SMILES: Cc1nn(C(C)C)cc1-c1nc2c3cccc(C(F)(F)F)c3nc(Cl)n2n1] |
| | 5-chloro-2-[3-methyl-1-(propan-2-yl)-1H-pyrazol-4-yl]-7- |
| | (trifluoromethyl)[1,2,4]triazolo[1,5-c]quinazoline |
| | LC-MS (Method 1): Rₜ = 1.42 min; MS (ESIpos): m/z = 395 [M + H]⁺ |
| | ¹H-NMR (400 MHz, DMSO-d₆): δ [ppm] = 1.47 (d, 6H), 2.60 (s, 3H), |
| | 4.56 (spt, 1H), 7.97 (t, 1H), 8.34 (dd, 1H), 8.47 (s, 1H), 8.73 (dd, 1H). |
| Intermediate 698 | 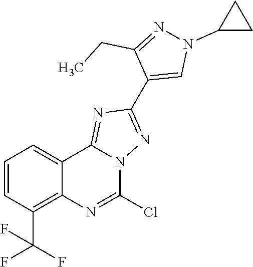 [SMILES: CCc1nn(C2CC2)cc1-c1nc2c3cccc(C(F)(F)F)c3nc(Cl)n2n1] |
| | 5-chloro-2-(1-cyclopropyl-3-ethyl-1H-pyrazol-4-yl)-7- |
| | (trifluoromethyl)[1,2,4]triazolo[1,5-c]quinazoline |
| | LC-MS (Method 1): Rₜ = 1.46 min; MS (ESIpos): m/z = 407 [M + H]⁺ |
| | ¹H-NMR (400 MHz, DMSO-d₆): δ [ppm] = 0.96-1.02 (m, 2H), 1.12- |
| | 1.17 (m, 2H), 1.29 (t, 3H), 3.04 (q, 2H), 3.80 (tt, 1H), 7.97 (t, 1H), |
| | 8.34 (dd, 1H), 8.46 (s, 1H), 8.71 (dd, 1H). |
| Intermediate 699 | 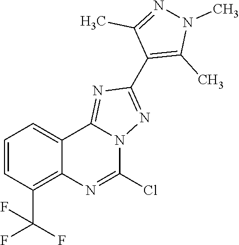 [SMILES: Cc1nn(C)c(C)c1-c1nc2c3cccc(C(F)(F)F)c3nc(Cl)n2n1] |
| | 5-chloro-7-(trifluoromethyl)-2-(1,3,5-trimethyl-1H-pyrazol-4- |
| | yl)[1,2,4]triazolo[1,5-c]quinazoline |
| | LC-MS (Method 1): Rₜ = 1.36 min; MS (ESIpos): m/z = 381 [M + H]⁺ |
| | ¹H-NMR (400 MHz, DMSO-d₆): δ [ppm] = 2.55 (s, 3H), 2.74 (s, 3H), |
| | 3.77 (s, 3H), 7.97 (t, 1H), 8.34 (dd, 1H), 8.77 (dd, 1H). |
| Intermediate 700 | 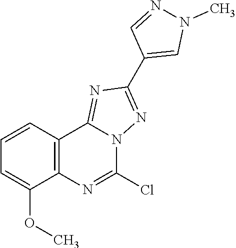 [SMILES: COc1cccc2c1nc(Cl)n1nc(-c3cnn(C)c3)nc21] |
| | 5-chloro-7-methoxy-2-(1-methyl-1H-pyrazol-4-yl)[1,2,4]triazolo[1,5- |
| | c]quinazoline |
| | LC-MS (Method 1): Rₜ = 0.89 min; MS (ESIpos): m/z = 315 [M + H]⁺ |
| | ¹H-NMR (400 MHz, DMSO-d₆): δ [ppm] = 3.95 (s, 3H), 4.01 (s, 3H), |
| | 7.50 (dd, 1H), 7.77 (t, 1H), 7.97 (dd, 1H), 8.08 (s, 1H), 8.53 (s, 1H). |
| Intermediate 701 | 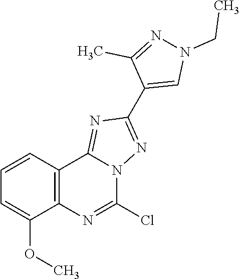 [SMILES: CCn1cc(-c2nc3c4cccc(OC)c4nc(Cl)n3n2)c(C)n1] |
| | 5-chloro-2-(1-ethyl-3-methyl-1H-pyrazol-4-yl)-7- |
| | methoxy[1,2,4]triazolo[1,5-c]quinazoline |
| | LC-MS (Method 1): Rₜ = 1.12 min; MS (ESIpos): m/z = 343 [M + H]⁺ |
| | ¹H-NMR (400 MHz, DMSO-d₆): δ [ppm] = 1.41 (t, 3H), 2.58 (s, 3H), |
| | 4.01 (s, 3H), 4.16 (q, 2H), 7.49 (dd, 1H), 7.76 (t, 1H), 7.95 (dd, 1H), |
| | 8.45 (s, 1H). |
| Intermediate 702 | 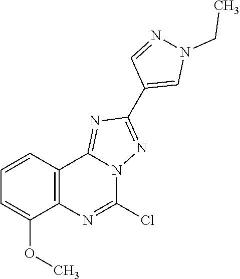 [SMILES: CCn1cc(-c2nc3c4cccc(OC)c4nc(Cl)n3n2)cn1] |
| | 5-chloro-2-(1-ethyl-1H-pyrazol-4-yl)-7-methoxy[1,2,4]triazolo[1,5- |
| | c]quinazoline |
| | LC-MS (Method 1): Rₜ = 1.01 min; MS (ESIpos): m/z = 329 [M + H]⁺ |
| | ¹H-NMR (400 MHz, DMSO-d₆): δ [ppm] = 1.45 (t, 3H), 4.01 (s, 3H), |
| | 4.25 (q, 2H), 7.49 (dd, 1H), 7.76 (t, 1H), 7.96 (dd, 1H), 8.10 (d, 1H), |
| | 8.57 (d, 1H). |
| Intermediate 703 | 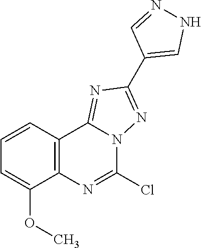 [SMILES: COc1cccc2c1nc(Cl)n1nc(-c3cn[nH]c3)nc21] |
| | 5-chloro-7-methoxy-2-(1H-pyrazol-4-yl)[1,2,4]triazolo[1,5-c]quinazoline |
| | LC-MS (Method 1): Rₜ = 0.84 min; MS (ESIpos): m/z = 301 [M + H]⁺ |
| Intermediate 704 | 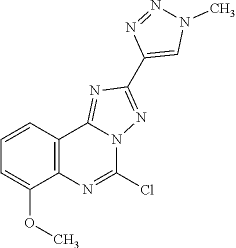 [SMILES: COc1cccc2c1nc(Cl)n1nc(-c3cn(C)nn3)nc21] |
| | 5-chloro-7-methoxy-2-(1-methyl-1H-1,2,3-triazol-4-yl)[1,2,4]triazolo[1,5- |
| | c]quinazoline |
| | LC-MS (Method 1): Rₜ = 0.84 min; MS (ESIpos): m/z = 316 [M + H]⁺ |
| | ¹H-NMR (400 MHz, DMSO-d₆): δ [ppm] = 4.02 (s, 3H), 4.18 (s, 3H), |
| | 7.53 (dd, 1H), 7.80 (t, 1H), 8.00 (dd, 1H), 8.90 (s, 1H). |
| Intermediate 705 | 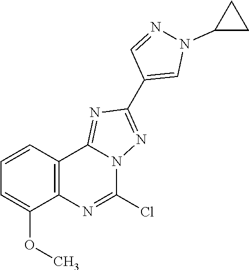 [SMILES: COc1cccc2c1nc(Cl)n1nc(-c3cnn(C4CC4)c3)nc21] |
| | 5-chloro-2-(1-cyclopropyl-1H-pyrazol-4-yl)-7-methoxy[1,2,4]triazolo[1,5- |
| | c]quinazoline |
| | LC-MS (Method 1): Rₜ = 1.05 min; MS (ESIpos): m/z = 341 [M + H]⁺ |
| | ¹H-NMR (400 MHz, DMSO-d₆): δ [ppm] = 0.99-1.06 (m, 2H), 1.14-1.20 |
| | (m, 2H), 3.89 (tt, 1H), 4.01 (s, 3H), 7.50 (dd, 1H), 7.77 (t, 1H), 7.97 |
| | (dd, 1H), 8.08 (s, 1H), 8.59 (s, 1H). |
| Intermediate 706 | 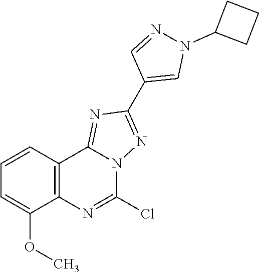 [SMILES: COc1cccc2c1nc(Cl)n1nc(-c3cnn(C4CCC4)c3)nc21] |
| | 5-chloro-2-(1-cyclobutyl-1H-pyrazol-4-yl)-7-methoxy[1,2,4]triazolo[1,5- |
| | c]quinazoline |
| | LC-MS (Method 1): Rₜ = 1.16 min; MS (ESIpos): m/z = 355 [M + H]⁺ |
| | ¹H-NMR (400 MHz, DMSO-d₆): δ [ppm] = 1.76-1.87 (m, 2H), 2.39-2.47 |
| | (m, 2H), 2.52-2.62 (m, 2H), 4.01 (s, 3H), 4.93-5.02 (m, 1H), 7.50 (dd, 1H), |
| | 7.77 (t, 1H), 7.97 (dd, 1H), 8.14 (d, 1H), 8.63 (d, 1H). |
| Intermediate 707 | 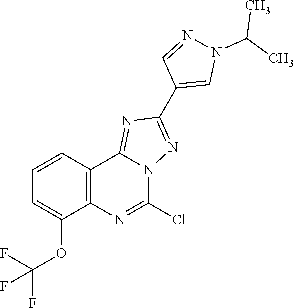 [SMILES: CC(C)n1cc(-c2nc3c4cccc(OC(F)(F)F)c4nc(Cl)n3n2)cn1] |
| | 5-chloro-2-[1-(propan-2-yl)-1H-pyrazol-4-yl]-7- |
| | (trifluoromethoxy)[1,2,4]triazolo[1,5-c]quinazoline |
| | LC-MS (Method 1): Rₜ = 1.34 min; MS (ESIpos): m/z = 397 [M + H]⁺ |
| | ¹H-NMR (400 MHz, DMSO-d₆): δ [ppm] = 1.50 (d, 6H), 4.65 (spt, 1H), |
| | 7.91 (t, 1H), 8.02 (ddq, 1H), 8.12 (d, 1H), 8.47 (dd, 1H), 8.59 (d, 1H). |
| Intermediate 708 | 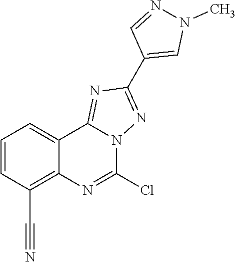 [SMILES: Cn1cc(-c2nc3c4cccc(C#N)c4nc(Cl)n3n2)cn1] |
| | 5-chloro-2-(1-methyl-1H-pyrazol-4-yl)[1,2,4]triazolo[1,5-c]quinazoline- |
| | 7-carbonitrile |
| | LC-MS (Method 1): Rₜ = min; 0.96 MS (ESIpos): m/z = 310 [M + H]⁺ |
| | ¹H-NMR (400 MHz, DMSO-d₆): δ [ppm] = 3.96 (s, 3H), 7.96 (t, 1H), |
| | 8.12 (d, 1H), 8.50 (dd, 1H), 8.57 (s, 1H), 8.74 (dd, 1H). |
| Intermediate 709 | 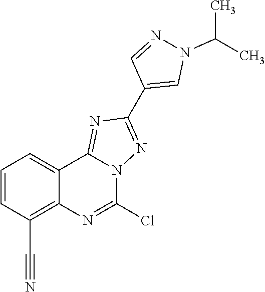 [SMILES: CC(C)n1cc(-c2nc3c4cccc(C#N)c4nc(Cl)n3n2)cn1] |
| | 5-chloro-2-[1-(propan-2-yl)-1H-pyrazol-4-yl][1,2,4]triazolo[1,5- |
| | c]quinazoline-7-carbonitrile |
| | LC-MS (Method 1): Rₜ = 1.13 min; MS (ESIpos): m/z = 338 [M + H]⁺ |
| | ¹H-NMR (400 MHz, DMSO-d₆): δ [ppm] = 1.50 (d, 6H), 4.65 (spt, 1H), |
| | 7.97 (t, 1H), 8.13 (d, 1H), 8.50 (dd, 1H), 8.60 (d, 1H), 8.74 (dd, 1H). |
| Intermediate 710 | 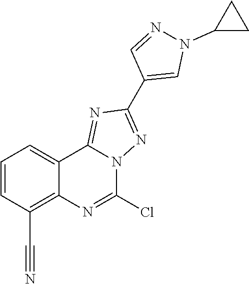 [SMILES: N#Cc1cccc2c1nc(Cl)n1nc(-c3cnn(C4CC4)c3)nc21] |
| | 5-chloro-2-(1-cyclopropyl-1H-pyrazol-4-yl)[1,2,4]triazolo[1,5- |
| | c]quinazoline-7-carbonitrile |
| | LC-MS (Method 1): Rₜ = 1.08 min; MS (ESIpos): m/z = 336 [M +H]⁺ |
| | ¹H-NMR (400 MHz, DMSO-d₆): δ [ppm] = 1.00-1.06 (m, 2H), 1.15- |
| | 1.22 (m, 2H), 3.90 (tt, 1H), 7.97 (t, 1H), 8.11 (d, 1H), 8.50 (dd, 1H), |
| | 8.63 (s, 1H), 8.73 (dd, 1H). |
| Intermediate 711 | 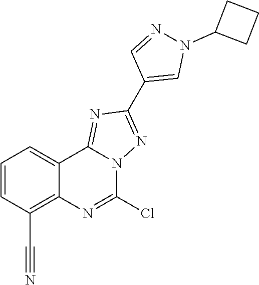 [SMILES: N#Cc1cccc2c1nc(Cl)n1nc(-c3cnn(C4CCC4)c3)nc21] |
| | 5-chloro-2-(1-cyclobutyl-1H-pyrazol-4-yl)[1,2,4]triazolo[1,5- |
| | c]quinazoline-7-carbonitrile |
| | LC-MS (Method 1): Rₜ = 1.18 min; MS (ESIpos): m/z = 350 [M + H]⁺ |
| | ¹H-NMR (400 MHz, DMSO-d₆): δ [ppm] = 1.76-1.88 (m, 2H), 2.39- |
| | 2.47 (m, 2H), 2.51-2.62 (m, 2H), 4.98 (br tt, 1H), 7.97 (t, 1H), 8.16 |
| | (d, 1H), 8.50 (dd, 1H), 8.67 (d, 1H), 8.74 (dd, 1H). |
| Intermediate 712 | 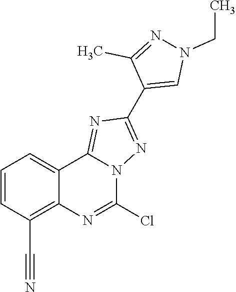 [SMILES: CCn1cc(-c2nc3c4cccc(C#N)c4nc(Cl)n3n2)c(C)n1] |
| | 5-chloro-2-(1-ethyl-3-methyl-1H-pyrazol-4-yl)[1,2,4]triazolo[1,5- |
| | c]quinazoline-7-carbonitrile |
| | LC-MS (Method 1): Rₜ = 1.14 min; MS (ESIpos): m/z = 338 [M + H]⁺ |
| | ¹H-NMR (400 MHz, DMSO-d₆): δ [ppm]= 1.42 (t, 3H), 2.59 (s, 3H), |
| | 4.17 (q, 2H), 7.96 (t, 1H), 8.47-8.51 (m, 2H), 8.72 (dd, 1H). |

### Intermediate 713 and Intermediate 714

### 5-chloro-2-[1-(difluoromethyl)-1H-pyrazol-4-yl][1,2,4]triazolo[1,5-c]quinazoline-7-carbonitrile and 5-chloro-2-(1H-pyrazol-4-yl)[1,2,4]triazolo[1,5-c]quinazoline-7-carbonitrile

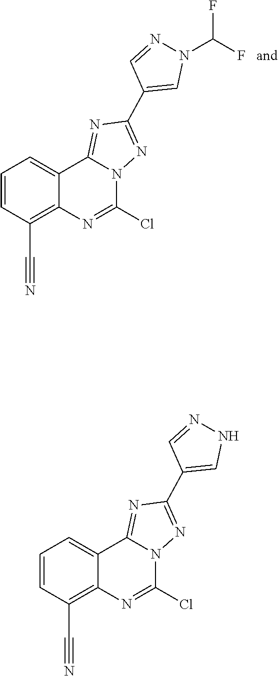 [SMILES: N#Cc1cccc2c1nc(Cl)n1nc(-c3cnn(C(F)F)c3)nc21 ; N#Cc1cccc2c1nc(Cl)n1nc(-c3cn[nH]c3)nc21]

A mixture of 2-[1-(difluoromethyl)-1H-pyrazol-4-yl]-5-oxo-5,6-dihydro[1,2,4]triazolo[1,5-c]quinazoline-7-carbonitrile and 5-oxo-2-(1H-pyrazol-4-yl)-5,6-dihydro[1,2,4]triazolo[1,5-c]quinazoline-7-carbonitrile (249 mg, about 761 μmol) was solubilised in phosphorus(V) oxychloride (2.1 mL, 23 mmol), N,N-diisopropylethylamine (4.0 mL, 23 mmol) was added carefully and the mixture was stirred for 2 h at 120° C. The mixture was cooled to rt and poured carefully into icewater and stirred for 30 minutes. Precipitated product was filtered off and dried to give 350 mg of about a 1:1 mixture of both title compounds.

LC-MS (Method 1):

Rₜ=1.10 min; MS (ESIpos): m/z=346 [M+H]⁺

### 5-chloro-2-[1-(difluoromethyl)-1H-pyrazol-4-yl][1,2,4]triazolo[1,5-c]quinazoline-7-carbonitrile

Rₜ=0.87 min; MS (ESIpos): m/z=296 [M+H]⁺

### 5-chloro-2-(1H-pyrazol-4-yl)[1,2,4]triazolo[1,5-c]quinazoline-7-carbonitrile

### Intermediate 715

### 5-chloro-2-(4-methoxyphenyl)-7-(methylsulfanyl)[1,2,4]triazolo[1,5-c]quinazoline

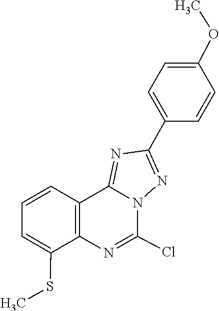 [SMILES: COc1ccc(-c2nc3c4cccc(SC)c4nc(Cl)n3n2)cc1]

2-(4-Methoxyphenyl)-7-(methylsulfanyl)[1,2,4]triazolo[1,5-c]quinazolin-5(6H)-one (154 mg, 455 μmol) was stirred in POCl3 (1.5 mL, 16 mmol) in the presence of N,N-diisopropylethylamine (790 μL, 4.6 mmol) for 2 h at 110° C. The mixture was cooled to rt and poured into ice. The solid was filtered, washed with water and dried at 60° C. under reduced pressure to give 173 mg of the title compound that was used without further purification.

LC-MS (method 2): Rₜ=1.46 min; MS (ESIpos): m/z=357 [M+H]⁺

¹H-NMR (400 MHz, DMSO-d6): δ [ppm]=2.57 (s, 3H), 3.87 (s, 3H), 7.13-7.18 (m, 2H), 7.68-7.72 (m, 1H), 7.77-7.82 (m, 1H), 8.19-8.26 (m, 3H).

The following intermediates were prepare similarly to intermediate 715

| | Structure |
|---|---|
| | IUPAC-Name |
| | LC-MS (method): Retention time; Mass found |
| Example | ¹H-NMR |
| Inter- mediate 716 | 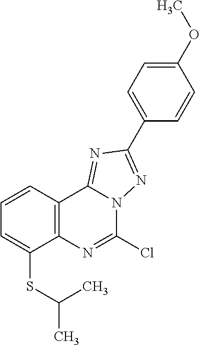 [SMILES: COc1ccc(-c2nc3c4cccc(SC(C)C)c4nc(Cl)n3n2)cc1] 5-chloro-2-(4-methoxyphenyl)-7- [(propan-2-yl)sulfanyl][1,2,4] triazolo[1,5-c]quinazoline LC-MS (Method 2): Rₜ = 1.53 min; MS (ESIpos): m/z = 385 [M + H]⁺ ¹H-NMR (400 MHz, DMSO-d₆): δ [ppm] = 1.39 (d, 6H), 3.76-3.86 (m, 1H), 3.86 (s, 3H), 7.14-7.18 (m, 2H), 7.75-7.80 (m, 1H), 7.83-7.86 (m, 1H), 8.22-8.26 (m, 3H). |
| Inter- mediate 717 | 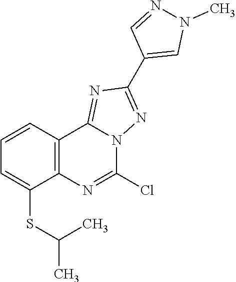 [SMILES: CC(C)Sc1cccc2c1nc(Cl)n1nc(-c3cnn(C)c3)nc21] 5-chloro-2-(1-methyl-1H-pyrazol-4-yl)-7- [(propan-2-yl)sulfanyl][1,2,4]triazolo [1,5-c]quinazoline LC-MS (Method 2): Rₜ = 1.22 min; MS (ESIpos): m/z = 359 [M + H]⁺ ¹H-NMR (400 MHz, DMSO-d₆): δ [ppm] = 1.38 (d, 6H), 3.81 (spt, 1H), 3.95 (s, 3H), 7.74-7.79 (m, 1H), 7.81-7.85 (m, 1H), 8.08 (d, 1H), 8.18 (dd, 1H), 8.53 (s, 1H). |
| Inter- mediate 718 | 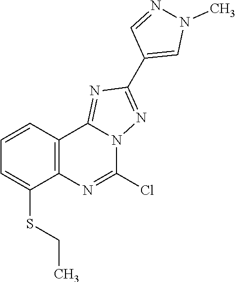 [SMILES: CCSc1cccc2c1nc(Cl)n1nc(-c3cnn(C)c3)nc21] 5-chloro-7-(ethylsulfanyl)-2-(1-methyl- 1H-pyrazol-4-yl)[1,2,4]triazolo[1,5-c] quinazoline LC-MS (Method 2): Rₜ = 1.15 min; MS (ESIpos): m/z = 345 [M + H]⁺ ¹H-NMR (400 MHz, DMSO-d₆): δ [ppm] = 1.36 (t, 3H), 3.12 (q, 2H), 3.96 (s, 3H), 7.74-7.77 (m, 2H), 8.09 (d, 1H), 8.13-8.18 (m, 1H), 8.53 (s, 1H). |
| Inter- mediate 719 | 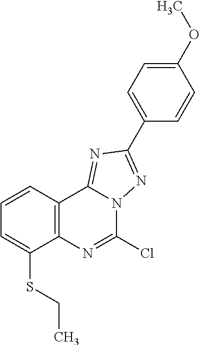 [SMILES: CCSc1cccc2c1nc(Cl)n1nc(-c3ccc(OC)cc3)nc21] 5-chloro-7-(ethylsulfanyl)-2-(4- methoxyphenyl)[1,2,4]triazolo[1,5- c]quinazoline LC-MS (Method 2): Rₜ = 1.47 min; MS (ESIpos): m/z = 371 [M + H]⁺ ¹H-NMR (400 MHz, DMSO-d₆): δ [ppm] = 1.37 (t, 3H), 3.13 (q, 2H), 3.86 (s, 3H), 7.15 (d, 2H), 7.74- 7.81 (m, 2H), 8.20-8.26 (m, 3H). |
| Inter mediate 720 | 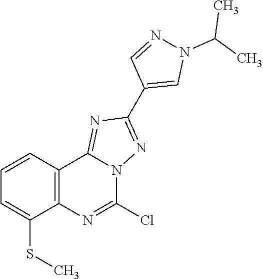 [SMILES: CSc1cccc2c1nc(Cl)n1nc(-c3cnn(C(C)C)c3)nc21] 5-chloro-7-(methylsulfanyl)-2- [1-(propan-2-yl)-1H-pyrazol-4- yl][1,2,4]triazolo[1,5-c]quinazoline LC-MS (Method 2): Rₜ = 1.26 min; MS (ESIpos): m/z = 359 [M + H]⁺ ¹H-NMR (400 MHz, DMSO-d₆): δ [ppm] = 1.50 (d, 6H), 2.57 (s, 3H), 4.64 (spt, 1H), 7.68-7.72 (m, 1H), 776-7.82 (m, 1H), 8.11 (s, 1H), 8.16 (dd, 1H), 8.57 (s, 1H). |
| Inter mediate 721 | 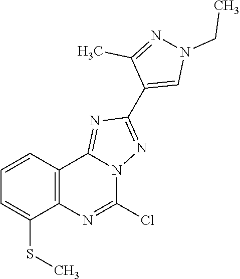 [SMILES: CCn1cc(-c2nc3c4cccc(SC)c4nc(Cl)n3n2)c(C)n1] 5-chloro-2-(1-ethyl-3-methyl-1H- pyrazol-4-yl)-7-(methylsulfanyl) [1,2,4]triazolo[1,5-c]quinazoline LC-MS (Method 2): Rₜ = 1.28 min; MS (ESIpos): m/z = 359 [M + H]⁺ ¹H-NMR (400 MHz, DMSO-d₆): δ [ppm] = 1.42 (t, 3H), 2.56 (s, 3H), 2.58 (s, 3H), 4.17 (q, 2H), 7.67-7.71 (m, 1H), 7.75-7.80 (m, 1H), 8.14 (dd, 1H), 8.45 (s, 1H). |

### Intermediate 722

### 7-bromo-5-chloro-2-(1-methyl-1H-pyrazol-5-yl)[1,2,4]triazolo[1,5-c]quinazoline

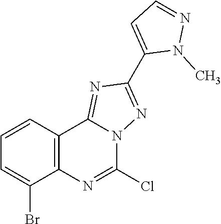 [SMILES: Cn1nccc1-c1nc2c3cccc(Br)c3nc(Cl)n2n1]

7-Bromo-2-(1-methyl-1H-pyrazol-5-yl)[1,2,4]triazolo[1,5-c]quinazolin-5(6H)-one (580 mg, 1.68 mmol) was suspended in phosphoric trichloride (8.0 mL, 85.8 mmol). N,N-Diisopropylethylamine (2.9 mL, 17 mmol) was added and it was stirred at 110° C. for 3 h. The reaction mixture was allowed to cool down to rt, poured into ice/water and stirred for some minutes. The precipitate was filtered, washed three times with water and dried under vacuum at 50° C. overnight yielding 600 mg (98%) of the title compound which was used without further purification in the next step.

LC-MS (Method 2): Rₜ=1.27 min; MS (ESIpos): m/z=363 [m+H]⁺

¹H-NMR (400 MHz, DMSO-d₆): δ [ppm]=4.35 (s, 3H), 7.09 (d, 1H), 7.65 (d, 1H), 7.77 (t, 1H), 8.33 (dd, 1H), 8.53 (dd, 1H).

The following intermediates were prepared analogously to intermediate 722

| | Structure |
|---|---|
| | IUPAC-Name |
| | LC-MS (method): Retention time; Mass found |
| Example | ¹H-NMR |
| Inter- mediate 723 | 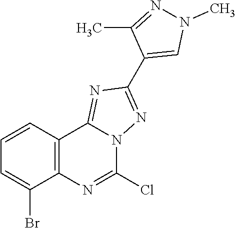 [SMILES: Cc1nn(C)cc1-c1nc2c3cccc(Br)c3nc(Cl)n2n1] 7-bromo-5-chloro-2-(1,3-dimethyl- 1H-pyrazol-4-yl)[1,2,4]triazolo[1,5- c]quinazoline LC-MS (Method 2): Rₜ = 1.21 min; MS (ESIpos): m/z = 379 [M + H]⁺ ¹H-NMR (400 MHz, DMSO-d₆): δ [ppm] = 2.58 (s, 3H), 3.88 (s, 3H), 7.73 (t, 1H), 8.29 (dd, 1H), 8.43-8.46 (m, 2H). |
| Inter- mediate 724 | 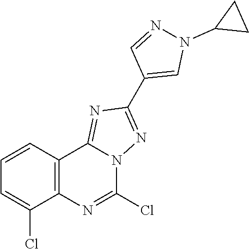 [SMILES: Clc1cccc2c1nc(Cl)n1nc(-c3cnn(C4CC4)c3)nc21] 5,7-dichloro-2-(1-cyclopropyl-1H- pyrazol-4-yl)[1,2,4]triazolo[1,5- c]quinazoline LC-MS (Method 2): Rₜ = 1.24 min; MS (ESIpos): m/z = 345 [M + H]⁺ ¹H-NMR (400 MHz, DMSO-d₆): δ [ppm] = 0.99-1.06 (m, 2H), 1.15-1.21 (m, 2H), 3.89 (tt, 1H), 7.80 (t, 1H), 8.09 (s, 1H), 8.11 (dd, 1H), 8.40 (dd, 1H), 8.60 (s, 1H). |
| Inter- mediate 725 | 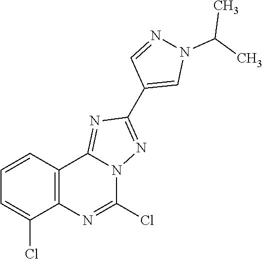 [SMILES: CC(C)n1cc(-c2nc3c4cccc(Cl)c4nc(Cl)n3n2)cn1] 5,7-dichloro-2-[1-(propan-2-yl)- 1H-pyrazol-4-yl][1,2,4]triazolo[1,5- c]quinazoline LC-MS (Method 2): Rₜ = 1.28 min; MS (ESIpos): m/z = 347 [M + H]⁺ ¹H-NMR (400 MHz, DMSO-d₆): δ [ppm] = 1.50 (d, 6H), 4.64 (spt, 1H), 7.80 (t, 1H), 8.07-8.16 (m, 2H), 8.41 (dd, 1H), 8.58 (s, 1H). |
| Inter- mediate 726 | 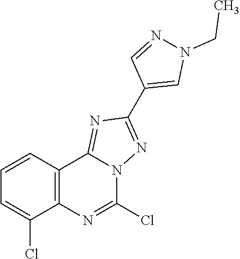 [SMILES: CCn1cc(-c2nc3c4cccc(Cl)c4nc(Cl)n3n2)cn1] 5,7-dichloro-2-(1-ethyl-1H-pyrazol-4- yl)[1,2,4]triazolo[1,5-c]quinazoline LC-MS (Method 2): Rₜ = 1.20 min; MS (ESIpos): m/z = 333 [M + H]⁺ ¹H-NMR (400 MHz, DMSO-d₆): δ [ppm] = 1.45 (t, 3H), 4.26 (q, 2H), 7.80 (t, 1H), 8.09-8.15 (m, 2H), 8.41 (dd, 1H), 8.58 (s, 1H). |
| Inter- mediate 727 | 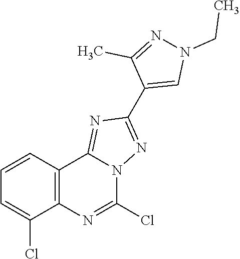 [SMILES: CCn1cc(-c2nc3c4cccc(Cl)c4nc(Cl)n3n2)c(C)n1] 5,7-dichloro-2-(1-ethyl-3-methyl- 1H-pyrazol-4-yl)[1,2,4] triazolo[1,5-c]quinazoline LC-MS (Method 2): Rₜ = 1.31 min; MS (ESIpos): m/z = 347 [M + H]⁺ |
| Inter- mediate 728 | 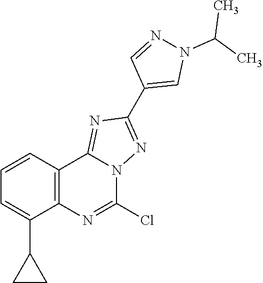 [SMILES: CC(C)n1cc(-c2nc3c4cccc(C5CC5)c4nc(Cl)n3n2)cn1] 5-chloro-7-cyclopropyl-2-[1-(propan- 2-yl)-1H-pyrazol-4-yl][1,2,4] triazolo[1,5-c]quinazoline LC-MS (Method 2): Rₜ = 1.36 min; MS (ESIpos): m/z = 353 [M + H]⁺ ¹H-NMR (400 MHz, DMSO-d₆): δ [ppm] = 0.90 (dd, 2H), 1.14-1.21 (m, 3H), 1.50 (d, 6H), 2.93-3.02 (m, 1H), 4.64 (spt, 1H), 7.40 (dd, 1H), 7.71 (t, 1H), 8.10 (s, 1H), 8.22 (dd, 1H), 8.56 (s, 1H). |
| Inter- mediate 729 | 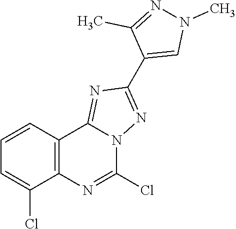 [SMILES: Cc1nn(C)cc1-c1nc2c3cccc(Cl)c3nc(Cl)n2n1] 5,7-dichloro-2-(1,3-dimethyl-1H- pyrazol-4-yl)[1,2,4]triazolo[1,5- c]quinazoline LC-MS (Method 2): Rₜ = 1.24 min; MS (ESIpos): m/z = 333 [M + H]⁺ |
| Inter- mediate 730 | 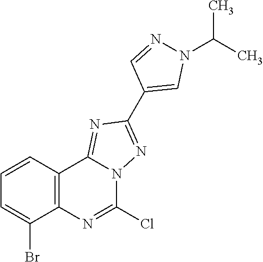 [SMILES: CC(C)n1cc(-c2nc3c4cccc(Br)c4nc(Cl)n3n2)cn1] 7-bromo-5-chloro-2-[1-(propan-2- yl)-1H-pyrazol-4-yl][1,2,4]triazolo[1,5- c]quinazoline LC-MS (Method 2): Rt = 1.26 min; MS (ESIpos): m/z = 391 [M + H]⁺ ¹H-NMR (400 MHz, DMSO-d₆): δ [ppm] = 1.50 (d, 6H), 4.65 (spt, 1H), 7.73 (t, 1H), 8.12 (s, 1H), 8.29 (dd, 1H), 8.46 (dd, 1H), 8.58 (s, 1H). |
| Inter- mediate 731 | 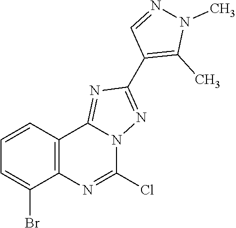 [SMILES: Cc1c(-c2nc3c4cccc(Br)c4nc(Cl)n3n2)cnn1C] 7-bromo-5-chloro-2-(1,5-dimethyl- 1H-pyrazol-4-yl)[1,2,4]triazolo[1,5- c]quinazoline LC-MS (Method 2): Rt = 1.25 min; MS (ESIpos): m/z = 377 [M + H]⁺ |
| Inter- mediate 732 | 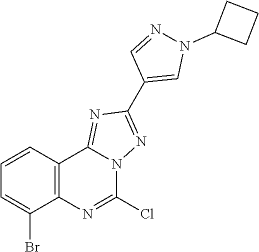 [SMILES: Clc1nc2c(Br)cccc2c2nc(-c3cnn(C4CCC4)c3)nn12] 7-bromo-5-chloro-2-(1-cyclobutyl- 1H-pyrazol-4-yl)[1,2,4]triazolo[1,5- c]quinazoline LC-MS (Method 2): Rt = 1.34 min; MS (ESIpos): m/z = 403 [M + H]⁺ |
| Inter- mediate 733 | 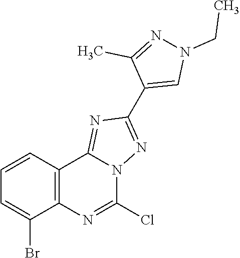 [SMILES: CCn1cc(-c2nc3c4cccc(Br)c4nc(Cl)n3n2)c(C)n1] 7-bromo-5-chloro-2-(1-ethyl-3-methyl- 1H-pyrazol-4-yl)[1,2,4]triazolo[1,5-c] quinazoline LC-MS (Method 2): Rt = 1.31 min; MS (ESIpos): m/z = 391 [M + H]⁺ |
| Inter- mediate 734 | 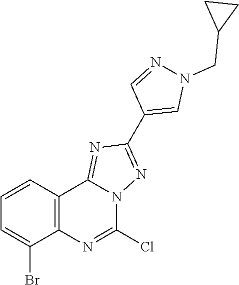 [SMILES: Clc1nc2c(Br)cccc2c2nc(-c3cnn(CC4CC4)c3)nn12] 7-bromo-5-chloro-2-[1-(cyclopropyl- methyl)-1H-pyrazol-4- yl][1,2,4]triazolo[1,5-c]quinazoline |
| Inter- mediate 735 | 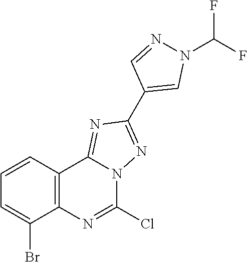 [SMILES: FC(F)n1cc(-c2nc3c4cccc(Br)c4nc(Cl)n3n2)cn1] 7-bromo-5-chloro-2-[1-(difluoromethyl)- 1H-pyrazol-4-yl][1,2,4]triazolo [1,5-c]quinazoline |
| Inter- mediate 736 | 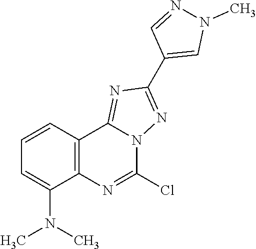 [SMILES: CN(C)c1cccc2c1nc(Cl)n1nc(-c3cnn(C)c3)nc21] 5-chloro-N,N-dimethyl-2-(1-methyl-1H- pyrazol-4-yl)[1,2,4]triazolo[1,5- c]quinazolin-7-amine LC-MS (Method 2): Rt = 1.10 min; MS (ESIpos): m/z = 328 [M + H]⁺ ¹H-NMR (400 MHz, DMSO-d₆): δ [ppm] = 3.07 (s, 6H), 3.95 (s, 3H), 7.31 (dd, 1H), 7.65-7.69 (m, 1H), 7.88 1H), 8.08 (d, 1H), 8.52 (s, 1H). |
| Inter- mediate 737 | 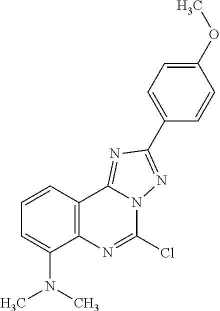 [SMILES: COc1ccc(-c2nc3c4cccc(N(C)C)c4nc(Cl)n3n2)cc1] 5-chloro-2-(4-methoxyphenyl)-N,N- dimethyl[1,2,4]triazolo[1,5- c]quinazolin-7-amine ¹H-NMR (400 MHz, DMSO-d₆): δ [ppm] = 3.08 (s, 6H), 3.86 (s, 3H), 7.12-7.17 (m, 2H), 7.31 (dd, 1H), 7.67 (t, 1H), 7.94 (dd, 1H), 8.19-8.26 (m, 2H) |
| Inter- mediate 738 | 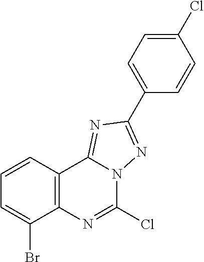 [SMILES: Clc1ccc(-c2nc3c4cccc(Br)c4nc(Cl)n3n2)cc1] 7-bromo-5-chloro-2-(4-chloro- phenyl)[1,2,4]triazolo[1,5-c] quinazoline LC-MS (Method 2): Rₜ = 1.45 min; MS (ESIpos): m/z = 393 [M + H]⁺ ¹H-NMR (400 MHz, DMSO-d₆): δ [ppm] = 7.66-7.72 (m, 2H), 7.77 (t, 1H), 8.29-8.34 (m, 3H), 8.53 (dd, 1H). |
| Inter- mediate 739 | 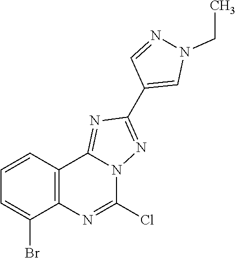 [SMILES: CCn1cc(-c2nc3c4cccc(Br)c4nc(Cl)n3n2)cn1] 7-bromo-5-chloro-2-(1-ethyl-1H- pyrazol-4-yl)[1,2,4]triazolo[1,5- c]quinazoline LC-MS (Method 2): Rt = 1.22 min; MS (ESIpos): m/z = 377 [M + H]⁺ |
| Inter- mediate 740 | 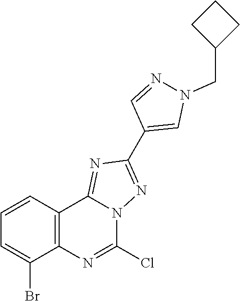 [SMILES: Clc1nc2c(Br)cccc2c2nc(-c3cnn(CC4CCC4)c3)nn12] 7-bromo-5-chloro-2-[1-(cyclo- butylmethyl)-1H-pyrazol-4- yl][1,2,4]triazolo[1,5-c]quinazoline LC-MS (Method 2): Rt = 1.40 min; MS (ESIpos): m/z = 417 [M + H]⁺ |
| Inter- mediate 741 | 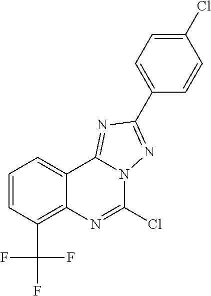 [SMILES: FC(F)(F)c1cccc2c1nc(Cl)n1nc(-c3ccc(Cl)cc3)nc21] 5-chloro-2-(4-chlorophenyl)-7- (trifluoromethyl)[1,2,4]triazolo[1,5- c]quinazoline LC-MS (Method 2): Rₜ = 1.58 min; MS (ESIpos): m/z = 383 [M + H]⁺ ¹H-NMR (400 MHz, DMSO-d₆): δ [ppm] = 7.67-7.71 (m, 2H), 8.01 (t, 1H), 8.29-8.34 (m, 2H), 8.38 (d, 1H), 8.79-8.83 (m, 1H). |
| Inter- mediate 742 | 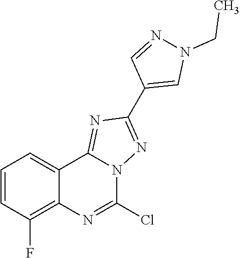 [SMILES: CCn1cc(-c2nc3c4cccc(F)c4nc(Cl)n3n2)cn1] 5-chloro-2-(1-ethyl-1H-pyrazol-4- yl)-7-fluoro[1,2,4]triazolo[1,5- c]quinazoline LC-MS (Method 2): Rₜ = 1.07 min; MS (ESIpos): m/z = 317 [M + H]⁺ ¹H-NMR (400 MHz, DMSO-d₆): δ [ppm] = 1.45 (t, 3H), 4.26 (q, 2H), 7.80- 7.84 (m, 1H), 7.85 (d, 1H), 8.10-8.12 (m, 1H), 8.26 (dd, 1H), 8.59 (s, 1H). |
| Inter- mediate 743 | 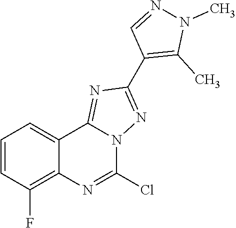 [SMILES: Cc1c(-c2nc3c4cccc(F)c4nc(Cl)n3n2)cnn1C] 5-chloro-2-(1,5-dimethyl-1H-pyrazol- 4-yl)-7-fluoro[1,2,4]triazolo[1,5- c]quinazoline LC-MS (Method 2): Rₜ = 1.10 min; MS (ESIpos): m/z = 317 [M + H]⁺ ¹H-NMR (400 MHz, DMSO-d₆): δ [ppm] = 2.75 (s, 3H), 3.85 (s, 3H), 7.81- 7.83 (m, 1H), 7.83-7.86 (m, 1H), 8.01 (s, 1H), 8.25-8.31 (m, 1H). |
| Inter- mediate 744 | 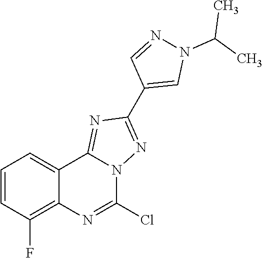 [SMILES: CC(C)n1cc(-c2nc3c4cccc(F)c4nc(Cl)n3n2)cn1] 5-chloro-7-fluoro-2-[1-(propan-2-yl)- 1H-pyrazol-4-yl][1,2,4]triazolo[1,5- c]quinazoline LC-MS (Method 2): Rₜ = 1.16 min; MS (ESIpos): m/z = 331 [M + H]⁺ ¹H-NMR (400 MHz, DMSO-d₆): δ [ppm] = 1.50 (d, 6H), 4.65 (spt, 1H), 7.81- 7.84 (m, 1H), 7.84-7.88 (m, 1H), 8.12 (s, 1H), 8.24-8.28 (m, 1H), 8.59 (s, 1H). |

### Intermediate 745

### N-[2-(1-methyl-1H-pyrazol-4-yl)-7-(trifluoromethyl)[1,2,4]triazolo[1,5-c]quinazolin-5-yl]-D-alanine

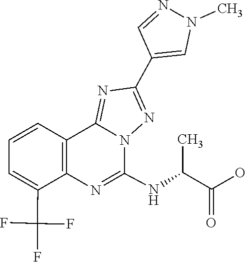

To a suspension of ethyl N-[2-(1-methyl-1H-pyrazol-4-yl)-7-(trifluoromethyl)[1,2,4]triazolo[1,5-c]quinazolin-5-yl]-D-alaninate (380 mg, 877 μmol) in ethanol (4.6 mL) was added a 2 M NaOH solution (1.8 mL) and this mixture was stirred for 1 h at rt. Then the reaction mixture was concentrated under reduced pressure and dissolved in water (10 mL). To this stirred aqueous solution 10% aqueous sulfuric acid was added up to acidic pH. The formed solid was collected via filtration, dried to give 316 mg (100% purity, 89% yield) of the target compound, which was used without further purification.

LC-MS (Method 2): Rₜ=0.69 min; MS (ESIpos): m/z=406 [M+H]⁺

¹H-NMR (400 MHz, DMSO-d₆): δ [ppm]=1.61 (d, 3H), 3.97 (s, 3H), 4.74 (dq, 1H), 7.53 (t, 1H), 8.05-8.09 (m, 2H), 8.44-8.48 (m, 2H), 8.49 (dd, 1H), 12.77 (br s, 1H).

### Intermediate 746 and Intermediate 747

### benzyl (6R)-6-({7-cyano-2-[1-(difluoromethyl)-1H-pyrazol-4-yl][1,2,4]triazolo[1,5-c]quinazolin-5-yl}amino)-5-oxo-1,4-diazepane-1-carboxylate and benzyl (6R)-6-{[7-cyano-2-(1H-pyrazol-4-yl)[1,2,4]triazolo[1,5-c]quinazolin-5-yl]amino}-5-oxo-1,4-diazepane-1-carboxylate

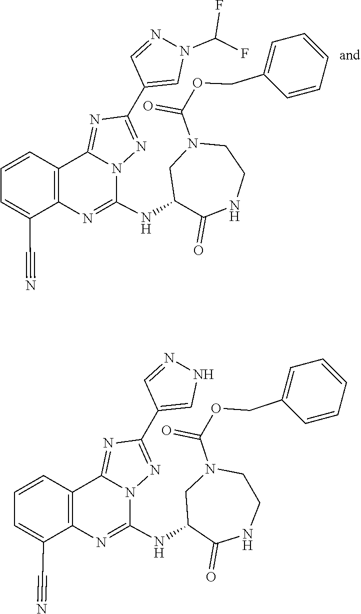 [SMILES: N#Cc1cccc2c1nc(N[C@@H]1CN(C(=O)OCc3ccccc3)CCNC1=O)n1nc(-c3cnn(C(F)F)c3)nc21 ; N#Cc1cccc2c1nc(N[C@@H]1CN(C(=O)OCc3ccccc3)CCNC1=O)n1nc(-c3cn[nH]c3)nc21]

A mixture of 5-chloro-2-[1-(difluoromethyl)-1H-pyrazol-4-yl][1,2,4]triazolo[1,5-c]quinazoline-7-carbonitrile and 5-chloro-2-(1H-pyrazol-4-yl)[1,2,4]triazolo[1,5-c]quinazoline-7-carbonitrile (293 mg, about 976 μmol), benzyl (6R)-6-amino-5-oxo-1,4-diazepane-1-carboxylate-hydrogen chloride (1/1) (293 mg, 976 μmol) and N,N-diisopropylethylamine (450 μL, 2.6 mmol) were stirred in DMSO (5.5 mL) for 1.5 h at 60° C. Water was added to the mixture, filtered, washed with water and dried under reduced pressure at 60° C. to give 129 mg of about a 1:1 mixture of both target compounds.

LC-MS (Method 1):

Rₜ=1.18 min; MS (ESIpos): m/z=573 [M+H]⁺

### benzyl (6R)-6-({7-cyano-2-[1-(difluoromethyl)-1H-pyrazol-4-yl][1,2,4]triazolo[1,5-c]quinazolin-5-yl}amino)-5-oxo-1,4-diazepane-1-carboxylate

Rₜ=1.01 min; MS (ESIpos): m/z=523 [MH]⁺

### benzyl (6R)-6-{[7-cyano-2-(1H-pyrazol-4-yl)[1,2,4]triazolo[1,5-c]quinazolin-5-yl]amino}-5-oxo-1,4-diazepane-1-carboxylate

### Intermediate 748

### benzyl (6R)-6-{[7-(methanesulfonyl)-2-(4-methoxyphenyl)[1,2,4]triazolo[1,5-c]quinazolin-5-yl]amino}-5-oxo-1,4-diazepane-1-carboxylate

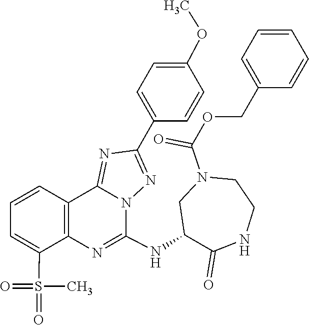 [SMILES: COc1ccc(-c2nc3c4cccc(S(C)(=O)=O)c4nc(N[C@@H]4CN(C(=O)OCc5ccccc5)CCNC4=O)n3n2)cc1]

Benzyl (6R)-6-{[7-bromo-2-(4-methoxyphenyl)[1,2,4]triazolo[1,5-c]quinazolin-5-yl]amino}-5-oxo-1,4-diazepane-1-carboxylate (300 mg, 487 μmol), copper(I) trifluoromethanesulfonate benzene complex (24.5 mg, 48.7 μmol) and sodium methanesulfinate (149 mg, 1.46 mmol) were solubilised in DMSO and trans-N,N-dimethylcyclohexane-1,2-diamine (31 μL, 190 μmol) was added. The reaction was stirred at 130° C. overnight. copper(I) trifluoromethanesulfonate benzene complex (24.5 mg, 48.7 μmol) and trans-N,N-dimethylcyclohexane-1,2-diamine (31 μL, 190 μmol) were added and the reaction was stirred for another 24 h. The reaction mixture was cooled to rt and poured into water and stirred for one hour. The solid was filtered and washed with water to give 250 mg of the title compound. The crude material was used without further purification.

LC-MS (Method 2): Rₜ=1.19 min; MS (ESIpos): m/z=614 [M+H]⁺

### Intermediate 749

### benzyl (6R)-6-{[7-(methanesulfonyl)-2-(1-methyl-1H-pyrazol-4-yl)[1,2,4]triazolo[1,5-c]quinazolin-5-yl]amino}-5-oxo-1,4-diazepane-1-carboxylate

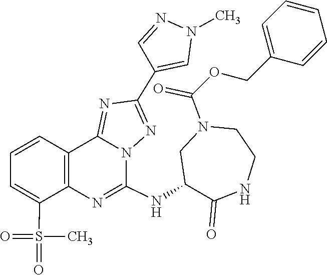 [SMILES: Cn1cc(-c2nc3c4cccc(S(C)(=O)=O)c4nc(N[C@@H]4CN(C(=O)OCc5ccccc5)CCNC4=O)n3n2)cn1]

The title compound was synthesised analogously to intermediate 748

LC-MS (method 2): Rₜ=0.96 min; MS (ESIneg): m/z=588 [M−H]⁻

### Intermediate 750

### benzyl (6R)-6-{[7-(ethylsulfanyl)-2-(1-methyl-1H-pyrazol-4-yl)[1,2,4]triazolo[1,5-c]quinazolin-5-yl]amino}-5-oxo-1,4-diazepane-1-carboxylate

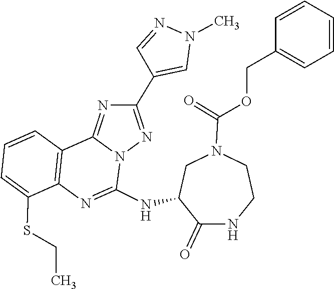 [SMILES: CCSc1cccc2c1nc(N[C@@H]1CN(C(=O)OCc3ccccc3)CCNC1=O)n1nc(-c3cnn(C)c3)nc21]

5-Chloro-7-(ethylsulfanyl)-2-(1-methyl-1H-pyrazol-4-yl)[1,2,4]triazolo[1,5-c]quinazoline (73.0 mg, 212 μmol), benzyl (6R)-6-amino-5-oxo-1,4-diazepane-1-carboxylate-hydrogen chloride (1/1) (69.8 mg, 233 μmol) and N,N-diisopropylethylamine (150 μL, 850 μmol) were stirred in DMSO (1.5 mL) for 2 h at 60° C. The mixture was cooled to rt and added dropwise to water (cooled with ice bath). The solid was filtered, washed with water and dried under reduced pressure at 60° C. to give 121 mg (90% purity, 90% yield) of the title compound that was used without further purification.

LC-MS (method 2): Rₜ=1.18 min; MS (ESIpos): m/z=572 [M+H]⁺

The following intermediates were prepared similarly to intermediate 750

| | Structure |
|---|---|
| | IUPAC-Name |
| | LC-MS (method): Retention time; Mass found |
| Example | ¹H-NMR |
| Inter- mediate 751 | 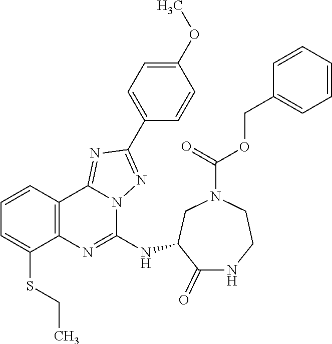 [SMILES: CCSc1cccc2c1nc(N[C@@H]1CN(C(=O)OCc3ccccc3)CCNC1=O)n1nc(-c3ccc(OC)cc3)nc21] benzyl (6R)-6-{[7-(ethylsulfanyl)-2-(4-methoxyphenyl) [1,2,4]triazolo[1,5-c]quinazolin-5-yl]amino}-5-oxo- 1,4-diazepane-1-carboxylate LC-MS (Method 2): Rₜ = 1.41 min; MS (ESIpos): m/z = 598 [M + H]⁺ |
| Inter- mediate 752 | 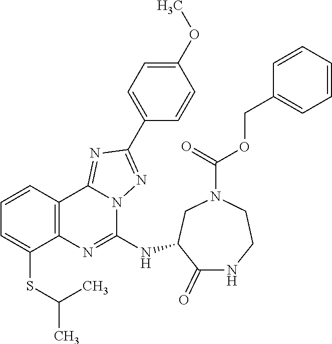 [SMILES: COc1ccc(-c2nc3c4cccc(SC(C)C)c4nc(N[C@@H]4CN(C(=O)OCc5ccccc5)CCNC4=O)n3n2)cc1] benzyl (6R)-6-({2-(4-methoxyphenyl)-7-[(propan-2- yl)sulfanyl][1,2,4]triazolo[1,5-c]quinazolin-5-yl} amino)-5-oxo-1,4-diazepane-1-carboxylate LC-MS (Method 2): Rₜ = 1.46 min; MS (ESIpos): m/z = 612 [M + H]⁺ |
| Inter- mediate 753 | 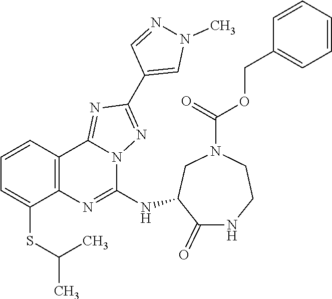 [SMILES: CC(C)Sc1cccc2c1nc(N[C@@H]1CN(C(=O)OCc3ccccc3)CCNC1=O)n1nc(-c3cnn(C)c3)nc21] benzyl (6R)-6-({2-(1-methyl-1H-pyrazol-4-yl)-7- [(propan-2-yl)sulfanyl][1,2,4]triazolo[1,5-c] quinazolin-5-yl}amino)-5-oxo-1,4-diazepane-1- carboxylate LC-MS (Method 2): Rₜ = 1.23 min; MS (ESIpos): m/z = 586 [M + H]⁺ |
| Inter- mediate 754 | 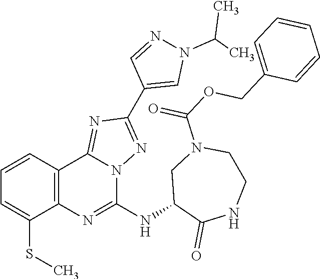 [SMILES: CSc1cccc2c1nc(N[C@@H]1CN(C(=O)OCc3ccccc3)CCNC1=O)n1nc(-c3cnn(C(C)C)c3)nc21] benzyl (6R)-6-({7-(methylsulfanyl)-2-[1-(propan-2-yl)- 1H-pyrazol-4-yl][1,2,4]triazolo[1,5-c]quinazolin- 5-yl}amino)-5-oxo-1,4-diazepane-1-carboxylate LC-MS (Method 2): Rₜ = 1.26 min; MS (ESIpos): m/z = 586 [M + H]⁺ |
| Inter- mediate 755 | 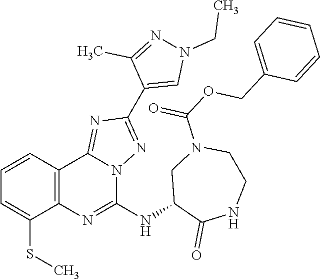 [SMILES: CCn1cc(-c2nc3c4cccc(SC)c4nc(N[C@@H]4CN(C(=O)OCc5ccccc5)CCNC4=O)n3n2)c(C)n1] benzyl (6R)-6-{[2-(1-ethyl-3-methyl-1H-pyrazol-4-yl)- 7-(methylsulfanyl)[1,2,4]triazolo[1,5-c]quinazolin- 5-yl]amino}-5-oxo-1,4-diazepane-1-carboxylate LC-MS (Method 2): Rₜ = 1.26 min; MS (ESIpos): m/z = 586 [M + H]⁺ |
| Inter- mediate 756 | 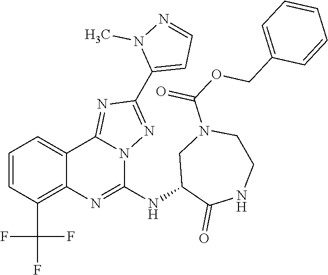 [SMILES: Cn1nccc1-c1nc2c3cccc(C(F)(F)F)c3nc(N[C@@H]3CN(C(=O)OCc4ccccc4)CCNC3=O)n2n1] benzyl (6R)-6-{[2-(1-methyl-1H-pyrazol-5-yl)-7- (trifluoromethyl)[1,2,4]triazolo[1,5-c]quinazolin- 5-yl]amino}-5-oxo-1,4-diazepane-1-carboxylate LC-MS (Method 2): Rₜ = 1.30 min; MS (ESIpos): m/z = 580 [M + H]⁺ ¹H-NMR (400 MHz, DMSO-d₆): δ [ppm] = 3.07-3.22 (m, 1H), 3.53-3.73 (m, 1H), 4.07 (br d, 1H), 4.18-4.42 (m, 4H), 4.87-5.21 (m, 3H), 6.96-7.45 (m, 6H), 7.57 (t, 1H), 7.63 (d, 1H), 8.11 (d, 1H), 8.26 (br s, 1H), 8.57 (d, 1H), 8.64 (br s, 1H). |
| Inter- mediate 757 | 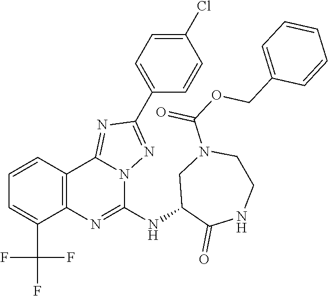 [SMILES: O=C1NCCN(C(=O)OCc2ccccc2)C[C@H]1Nc1nc2c(C(F)(F)F)cccc2c2nc(-c3ccc(Cl)cc3)nn12] benzyl (6R)-6-{[2-(4-chlorophenyl)-7-(trifluoromethyl) [1,2,4]triazolo[1,5-c]quinazolin-5-yl]amino}-5-oxo- 1,4-diazepane-1-carboxylate LC-MS (Method 2): Rt = 1.55 min; MS (ESIpos): m/z = 610 [M + H]⁺ |
| Inter- mediate 758 | 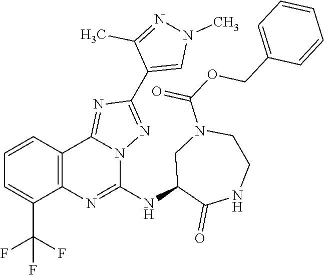 [SMILES: Cc1nn(C)cc1-c1nc2c3cccc(C(F)(F)F)c3nc(N[C@H]3CN(C(=O)OCc4ccccc4)CCNC3=O)n2n1] benzyl (6S)-6-{[2-(1,3-dimethyl-1H-pyrazol-4-yl)-7- (trifluoromethyl)[1,2,4]triazolo[1,5-c]quinazolin- 5-yl]amino}-5-oxo-1,4-diazepane-1-carboxylate LC-MS (Method 2): Rₜ = 1.23 min; MS (ESIpos): m/z = 594 [M + H]⁺ |
| Inter- mediate 759 | 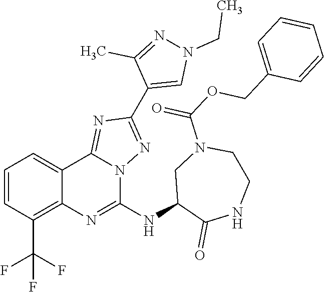 [SMILES: CCn1cc(-c2nc3c4cccc(C(F)(F)F)c4nc(N[C@H]4CN(C(=O)OCc5ccccc5)CCNC4=O)n3n2)c(C)n1] benzyl (6S)-6-{[2-(1-ethyl-3-methyl-1H-pyrazol-4-yl)- 7-(trifluoromethyl)[1,2,4]triazolo[1,5-c]quinazolin-5-yl] amino}-5-oxo-1,4-diazepane-1-carboxylate LC-MS (Method 2): Rₜ = 1.29 min; MS (ESIneg): m/z = 606 [M − H]⁻ |
| Inter- mediate 760 | 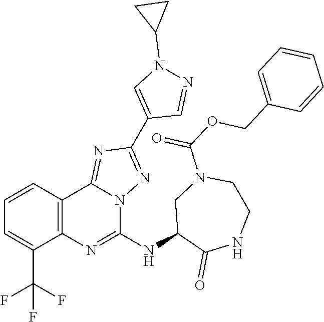 [SMILES: O=C1NCCN(C(=O)OCc2ccccc2)C[C@@H]1Nc1nc2c(C(F)(F)F)cccc2c2nc(-c3cnn(C4CC4)c3)nn12] benzyl (6S)-6-{[2-(1-cyclopropyl-1H-pyrazol-4-yl)-7- (trifluoromethyl)[1,2,4]triazolo[1,5-c]quinazolin-5- yl]amino}-5-oxo-1,4-diazepane-1-carboxylate LC-MS (Method 2): Rₜ = 1.27 min; MS (ESIpos): m/z = 606 [M + H]⁺ |
| Inter- mediate 761 | 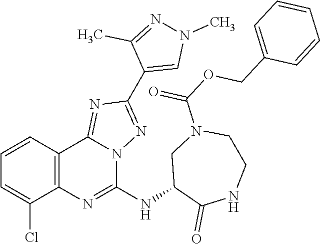 [SMILES: Cc1nn(C)cc1-c1nc2c3cccc(Cl)c3nc(N[C@@H]3CN(C(=O)OCc4ccccc4)CCNC3=O)n2n1] benzyl (6R)-6-{[7-chloro-2-(1,3-dimethyl-1H-pyrazol- 4-yl)[1,2,4]triazolo[1,5-c]quinazolin-5-yl]amino}-5- oxo-1,4-diazepane-1-carboxylate LC-MS (Method 2): Rₜ = min; MS (ESIneg): m/z = 558 [M − H]⁻ |
| Inter- mediate 762 | 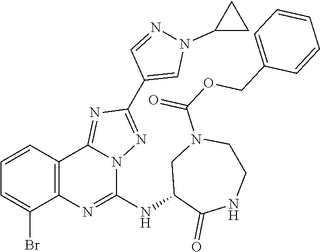 [SMILES: O=C1NCCN(C(=O)OCc2ccccc2)C[C@H]1Nc1nc2c(Br)cccc2c2nc(-c3cnn(C4CC4)c3)nn12] benzyl (6R)-6-{[7-bromo-2-(1-cyclopropyl-1H- pyrazol-4-yl)[1,2,4]triazolo[1,5-c]quinazolin-5-yl] amino}-5-oxo-1,4-diazepane-1-carboxylate LC-MS (Method 2): Rₜ = 1.25 min; MS (ESIneg): m/z = 614 [M − H]⁻ |
| Inter- mediate 763 | 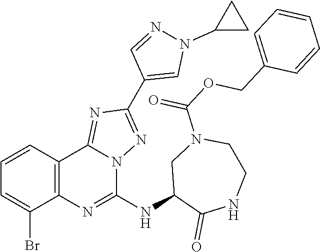 [SMILES: O=C1NCCN(C(=O)OCc2ccccc2)C[C@@H]1Nc1nc2c(Br)cccc2c2nc(-c3cnn(C4CC4)c3)nn12] benzyl (6S)-6-{[7-bromo-2-(1-cyclopropyl-1H- pyrazol-4-yl)[1,2,4]triazolo[1,5-c]quinazolin-5-yl] amino}-5-oxo-1,4-diazepane-1-carboxylate LC-MS (Method 2): Rₜ = 1.25 min; MS (ESIneg): m/z = 614 [M − H]⁻ |
| Inter- mediate 764 | 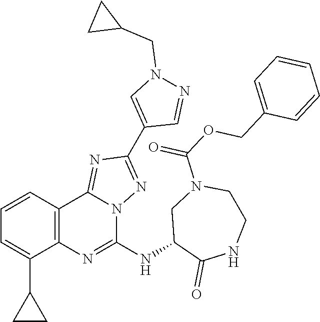 [SMILES: O=C1NCCN(C(=O)OCc2ccccc2)C[C@H]1Nc1nc2c(C3CC3)cccc2c2nc(-c3cnn(CC4CC4)c3)nn12] benzyl (6R)-6-({7-cyclopropyl-2-[1-(cyclopropyl- methyl)-1H-pyrazol-4-yl][1,2,4]triazolo[1,5-c] quinazolin-5-yl}amino)-5-oxo-1,4-diazepane-1- carboxylate LC-MS (Method 2): Rₜ = 1.34 min; MS (ESIpos): m/z = 592 [M + H]⁺ |
| Inter- mediate 765 | 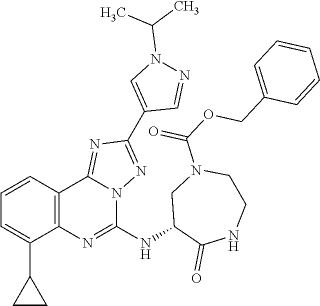 [SMILES: CC(C)n1cc(-c2nc3c4cccc(C5CC5)c4nc(N[C@@H]4CN(C(=O)OCc5ccccc5)CCNC4=O)n3n2)cn1] benzyl (6R)-6-({7-cyclopropyl-2-[1-(propan-2-yl)-1H- pyrazol-4-yl][1,2,4]triazolo[1,5-c]quinazolin-5-yl} amino)-5-oxo-1,4-diazepane-1-carboxylate LC-MS (Method 2): Rₜ = 1.33 min; MS (ESIpos): m/z = 580 [M + H]⁺ |
| Inter- mediate 766 | 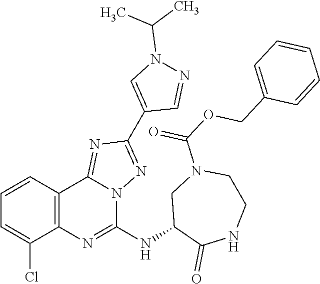 [SMILES: CC(C)n1cc(-c2nc3c4cccc(Cl)c4nc(N[C@@H]4CN(C(=O)OCc5ccccc5)CCNC4=O)n3n2)cn1] benzyl (6R)-6-({7-chloro-2-[1-(propan-2-yl)-1H- pyrazol-4-yl][1,2,4]triazolo[1,5-c]quinazolin-5-yl} amino)-5-oxo-1,4-diazepane-1-carboxylate LC-MS (Method 2): Rₜ = 1.31 min; MS (ESIpos): m/z = 574 [M + H]⁺ |
| Inter- mediate 767 | 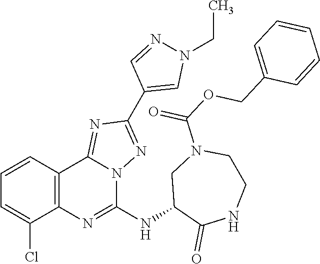 [SMILES: CCn1cc(-c2nc3c4cccc(Cl)c4nc(N[C@@H]4CN(C(=O)OCc5ccccc5)CCNC4=O)n3n2)cn1] benzyl (6R)-6-{[7-chloro-2-(1-ethyl-1H-pyrazol-4-yl) [1,2,4]triazolo[1,5-c]quinazolin-5-yl]amino}- 5-oxo-1,4-diazepane-1-carboxylate LC-MS (Method 2): Rₜ = 1.25 min; MS (ESIpos): m/z = 560 [M + H]⁺ |
| Inter- mediate 768 | 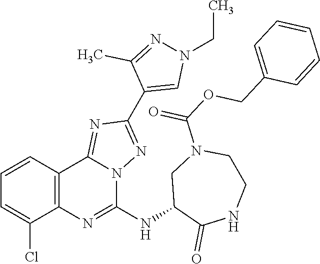 [SMILES: CCn1cc(-c2nc3c4cccc(Cl)c4nc(N[C@@H]4CN(C(=O)OCc5ccccc5)CCNC4=O)n3n2)c(C)n1] benzyl (6R)-6-{[7-chloro-2-(1-ethyl-3-methyl-1H- pyrazol-4-yl)[1,2,4]triazolo[1,5-c]quinazolin-5-yl] amino}-5-oxo-1,4-diazepane-1-carboxylate LC-MS (Method 2): Rₜ = 1.31 min; MS (ESIpos): m/z = 574 [M + H]⁺ |
| Inter- mediate 769 | 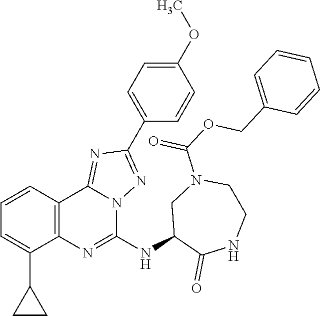 [SMILES: COc1ccc(-c2nc3c4cccc(C5CC5)c4nc(N[C@H]4CN(C(=O)OCc5ccccc5)CCNC4=O)n3n2)cc1] benzyl (6S)-6-{[7-cyclopropyl-2-(4-methoxyphenyl) [1,2,4]triazolo[1,5-c]quinazolin-5-yl]amino}- 5-oxo-1,4-diazepane-1-carboxylate LC-MS (Method 2): Rt = 1.46 min; MS (ESIpos): m/z = 578 [M + H]⁺ |
| Inter- mediate 770 | 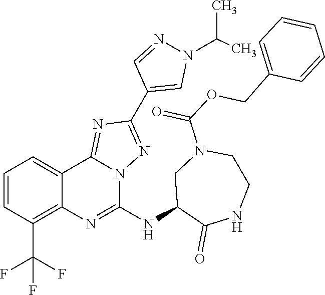 [SMILES: CC(C)n1cc(-c2nc3c4cccc(C(F)(F)F)c4nc(N[C@H]4CN(C(=O)OCc5ccccc5)CCNC4=O)n3n2)cn1] benzyl (6S)-5-oxo-6-({2-[1-(propan-2-yl)-1H-pyrazol- 4-yl]-7-(trifluoromethyl)[1,2,4]triazolo[1,5-c] quinazolin-5-yl}amino)-1,4-diazepane-1-carboxylate LC-MS (Method 2): Rₜ 1.30 = min; MS (ESIpos): m/z = 608 [M + H]⁺ |
| Inter- mediate 771 | 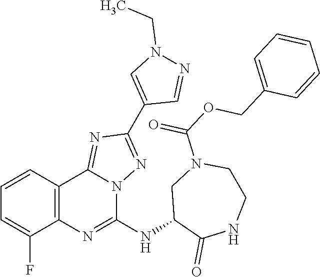 [SMILES: CCn1cc(-c2nc3c4cccc(F)c4nc(N[C@@H]4CN(C(=O)OCc5ccccc5)CCNC4=O)n3n2)cn1] benzyl (6R)-6-{[2-(1-ethyl-1H-pyrazol-4-yl)-7-fluoro [1,2,4]triazolo[1,5-c]quinazolin-5-yl]amino}- 5-oxo-1,4-diazepane-1-carboxylate LC-MS (Method 2): Rₜ = 1.18 min; MS (ESIpos): m/z = 544 [M + H]⁺ |
| Inter- mediate 772 | 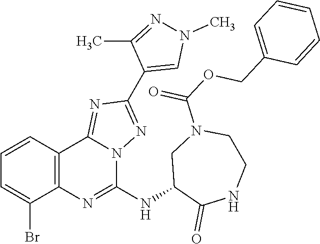 [SMILES: Cc1nn(C)cc1-c1nc2c3cccc(Br)c3nc(N[C@@H]3CN(C(=O)OCc4ccccc4)CCNC3=O)n2n1] benzyl (6R)-6-{[7-bromo-2-(1,3-dimethyl-1H-pyrazol-4- yl)[1,2,4]triazolo[1,5-c]quinazolin-5-yl]amino}-5-oxo- 1,4-diazepane-1-carboxylate LC-MS (Method 2): Rₜ = 1.22 min; MS (ESIpos): m/z = 606 [M + H]⁺ |
| Inter- mediate 773 | 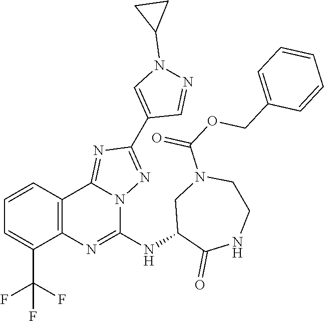 [SMILES: O=C1NCCN(C(=O)OCc2ccccc2)C[C@H]1Nc1nc2c(C(F)(F)F)cccc2c2nc(-c3cnn(C4CC4)c3)nn12] benzyl (6R)-6-{[2-(1-cyclopropyl-1H-pyrazol-4-yl)-7- (trifluoromethyl)[1,2,4]triazolo[1,5-c]quinazolin-5-yl] amino}-5-oxo-1,4-diazepane-1-carboxylate LC-MS (Method 2): Rₜ = 1.32 min; MS (ESIpos): m/z = 606 [M + H]⁺ |
| Inter- mediate 774 | 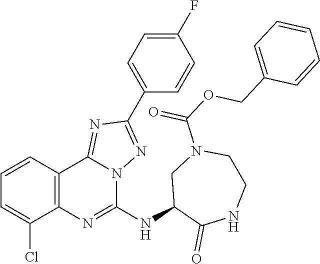 [SMILES: O=C1NCCN(C(=O)OCc2ccccc2)C[C@@H]1Nc1nc2c(Cl)cccc2c2nc(-c3ccc(F)cc3)nn12] benzyl (6S)-6-{[7-chloro-2-(4-fluorophenyl)[1,2,4] triazolo[1,5-c]quinazolin-5-yl]amino}-5-oxo- 1,4-diazepane-1-carboxylate LC-MS (Method 2): Rt = 1.43 min; MS (ESIpos): m/z = 560 [M + H]⁺ |
| Inter- mediate 775 | 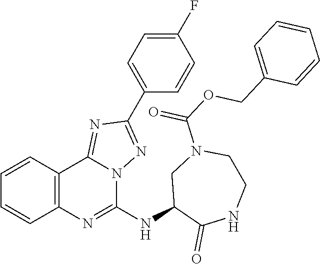 [SMILES: O=C1NCCN(C(=O)OCc2ccccc2)C[C@@H]1Nc1nc2ccccc2c2nc(-c3ccc(F)cc3)nn12] benzyl (6S)-6-{[2-(4-fluorophenyl)[1,2,4]triazolo [1,5-c]quinazolin-5-yl]amino}-5-oxo-1,4-diazepane- 1-carboxylate LC-MS (Method 2): Rₜ = 1.39 min; MS (ESIpos): m/z = 526 [M + H]⁺ |
| Inter- mediate 776 | 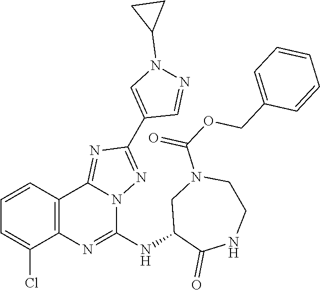 [SMILES: O=C1NCCN(C(=O)OCc2ccccc2)C[C@H]1Nc1nc2c(Cl)cccc2c2nc(-c3cnn(C4CC4)c3)nn12] benzyl (6R)-6-{[7-chloro-2-(1-cyclopropyl-1H- pyrazol-4-yl)[1,2,4]triazolo[1,5-c]quinazolin-5-yl] amino}-5-oxo-1,4-diazepane-1-carboxylate LC-MS (Method 2): Rₜ = 1.28 min; MS (ESIpos): m/z = 572 [M + H]⁺ |
| Inter- mediate 777 | 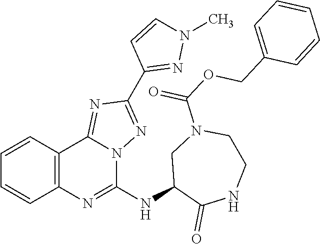 [SMILES: Cn1ccc(-c2nc3c4ccccc4nc(N[C@H]4CN(C(=O)OCc5ccccc5)CCNC4=O)n3n2)n1] benzyl (6S)-6-{[2-(1-methyl-1H-pyrazol-3-yl)[1,2,4] triazolo[1,5-c]quinazolin-5-yl]amino}-5-oxo-1,4- diazepane-1-carboxylate LC-MS (Method 2): Rₜ = 1.10 min; MS (ESIpos): m/z = 512 [M + H]⁺ |
| Inter- mediate 778 | 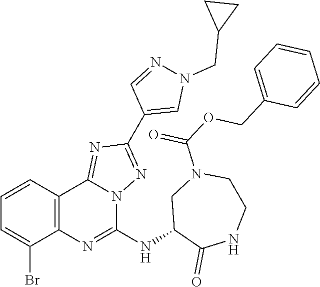 [SMILES: O=C1NCCN(C(=O)OCc2ccccc2)C[C@H]1Nc1nc2c(Br)cccc2c2nc(-c3cnn(CC4CC4)c3)nn12] benzyl (6R)-6-({7-bromo-2-[1-(cyclopropylmethyl)-1H- pyrazol-4-yl][1,2,4]triazolo[1,5-c]quinazolin-5-yl} amino)-5-oxo-1,4-diazepane-1-carboxylate LC-MS (Method 2): Rt = 1.30 min; MS (ESIpos): m/z = 630 [M + H]⁺ |
| Inter- mediate 779 | 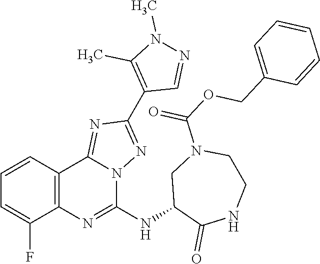 [SMILES: Cc1c(-c2nc3c4cccc(F)c4nc(N[C@@H]4CN(C(=O)OCc5ccccc5)CCNC4=O)n3n2)cnn1C] benzyl (6R)-6-{[2-(1,5-dimethyl-1H-pyrazol-4-yl)-7- fluoro[1,2,4]triazolo[1,5-c]quinazolin-5-yl]amino}-5- oxo-1,4-diazepane-1-carboxylate LC-MS (Method 2): Rₜ = 1.17 min; MS (ESIpos): m/z = 544 [M + H]⁺ |
| Inter- mediate 780 | 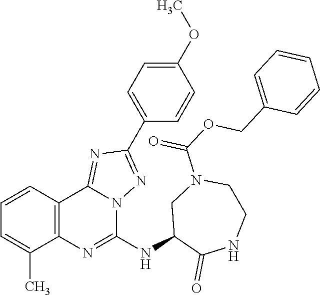 [SMILES: COc1ccc(-c2nc3c4cccc(C)c4nc(N[C@H]4CN(C(=O)OCc5ccccc5)CCNC4=O)n3n2)cc1] benzyl (6S)-6-{[2-(4-methoxyphenyl)-7-methyl[1,2,4] triazolo[1,5-c]quinazolin-5-yl]amino}-5-oxo- 1,4-diazepane-1-carboxylate LC-MS (Method 2): Rₜ = 1.42 min; MS (ESIpos): m/z = 552 [M + H]⁺ |
| Inter- mediate 781 | 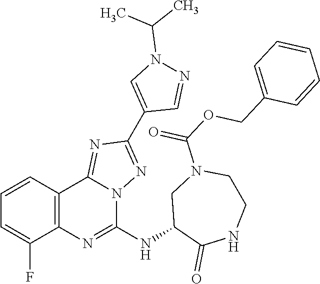 [SMILES: CC(C)n1cc(-c2nc3c4cccc(F)c4nc(N[C@@H]4CN(C(=O)OCc5ccccc5)CCNC4=O)n3n2)cn1] benzyl (6R)-6-({7-fluoro-2-[1-(propan-2-yl)-1H- pyrazol-4-yl][1,2,4]triazolo[1,5-c]quinazolin-5- yl}amino)-5-oxo-1,4-diazepane-1-carboxylate LC-MS (Method 2): Rₜ = 1.24 min; MS (ESIpos): m/z = 558 [M + H]⁺ |
| Inter- mediate 782 | 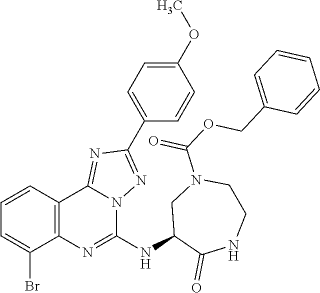 [SMILES: COc1ccc(-c2nc3c4cccc(Br)c4nc(N[C@H]4CN(C(=O)OCc5ccccc5)CCNC4=O)n3n2)cc1] benzyl (6S)-6-{[7-bromo-2-(4-methoxyphenyl)[1,2,41 triazolo[1,5-c]quinazolin-5-yl]amino}-5- oxo-1,4-diazepane-1-carboxylate LC-MS (Method 2): Rₜ = 1.41 min; MS (ESIpos): m/z = 618 [M + H]⁺ |
| Inter- mediate 783 | 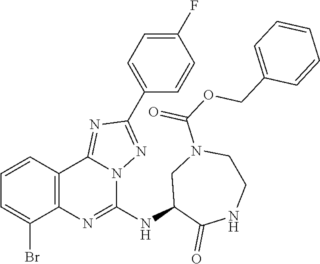 [SMILES: O=C1NCCN(C(=O)OCc2ccccc2)C[C@@H]1Nc1nc2c(Br)cccc2c2nc(-c3ccc(F)cc3)nn12] benzyl (6S)-6-{[7-bromo-2-(4-fluorophenyl)[1,2,4] triazolo[1,5-c]quinazolin-5-yl]amino}-5-oxo-1,4- diazepane-1-carboxylate LC-MS (Method 2): Rt = 1.45 min; MS (ESIpos): m/z = 604 [M + H]⁺ |
| Inter- mediate 784 | 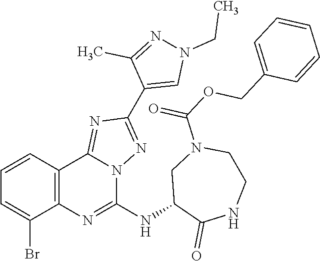 [SMILES: CCn1cc(-c2nc3c4cccc(Br)c4nc(N[C@@H]4CN(C(=O)OCc5ccccc5)CCNC4=O)n3n2)c(C)n1] benzyl (6R)-6-{[7-bromo-2-(1-ethyl-3-methyl-1H- pyrazol-4-yl)[1,2,4]triazolo[1,5-c]quinazolin-5-yl] amino}-5-oxo-1,4-diazepane-1-carboxylate LC-MS (Method 2): Rₜ = 1.31 min; MS (ESIpos): m/z = 620 [M + H]⁺ |
| Inter- mediate 785 | 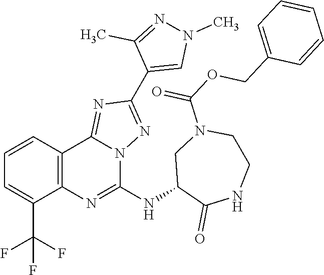 [SMILES: Cc1nn(C)cc1-c1nc2c3cccc(C(F)(F)F)c3nc(N[C@@H]3CN(C(=O)OCc4ccccc4)CCNC3=O)n2n1] benzyl (6R)-6-{[2-(1,3-dimethyl-1H-pyrazol-4-yl)-7- (trifluoromethyl)[1,2,4]triazolo[1,5-c]quinazolin-5-yl] amino}-5-oxo-1,4-diazepane-1-carboxylate LC-MS (Method 2): Rₜ = 1.23 min; MS (ESIpos): m/z = 594 [M + H]⁺ |
| Inter- mediate 786 | 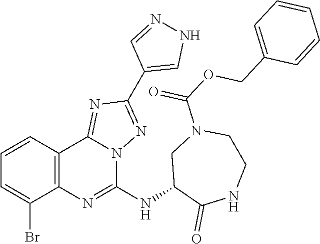 [SMILES: O=C1NCCN(C(=O)OCc2ccccc2)C[C@H]1Nc1nc2c(Br)cccc2c2nc(-c3cn[nH]c3)nn12] benzyl (6R)-6-{[7-bromo-2-(1H-pyrazol-4-yl)[1,2,4] triazolo[1,5-c]quinazolin-5-yl]amino}-5-oxo-1,4- diazepane-1-carboxylate LC-MS (Method 2): Rt = 1.13 min; MS (ESIpos): m/z = 576 [M + H]⁺ |
| Inter- mediate 787 | 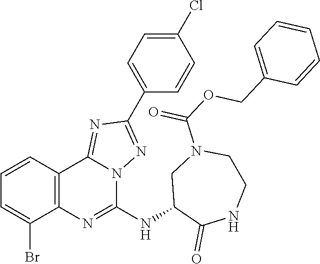 [SMILES: O=C1NCCN(C(=O)OCc2ccccc2)C[C@H]1Nc1nc2c(Br)cccc2c2nc(-c3ccc(Cl)cc3)nn12] benzyl (6R)-6-{[7-bromo-2-(4-chlorophenyl)[1,2,4] triazolo[1,5-c]quinazolin-5-yl]amino}-5-oxo-1,4- diazepane-1-carboxylate LC-MS (Method 2): Rₜ = 1.54 min; MS (ESIpos): m/z = 621 [M + H]⁺ |
| Inter- mediate 788 | 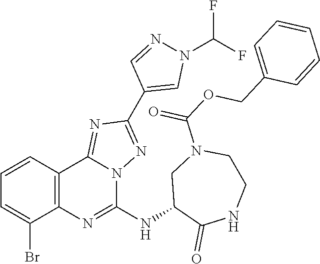 [SMILES: O=C1NCCN(C(=O)OCc2ccccc2)C[C@H]1Nc1nc2c(Br)cccc2c2nc(-c3cnn(C(F)F)c3)nn12] benzyl (6R)-6-({7-bromo-2-[1-(difluoromethyl)-1H- pyrazol-4-yl][1,2,4]triazolo[1,5-c]quinazolin-5-yl} amino)-5-oxo-1,4-diazepane-1-carboxylate LC-MS (Method 2): Rt = 1.29 min; MS (ESIpos): m/z = 626 [M + H]⁺ |
| Inter- mediate 789 | 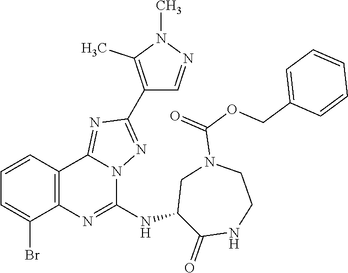 [SMILES: Cc1c(-c2nc3c4cccc(Br)c4nc(N[C@@H]4CN(C(=O)OCc5ccccc5)CCNC4=O)n3n2)cnn1C] benzyl (6R)-6-{[7-bromo-2-(1,5-dimethyl-1H-pyrazol-4- yl)[1,2,41triazolo[1,5-c]quinazolin-5-yl]amino}-5-oxo- 1,4-diazepane-1-carboxylate LC-MS (Method 2): Rt = 1.25 min; MS (ESIpos): m/z = 604 [M + H]⁺ |
| Inter- mediate 790 | 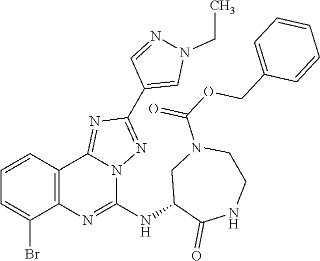 [SMILES: CCn1cc(-c2nc3c4cccc(Br)c4nc(N[C@@H]4CN(C(=O)OCc5ccccc5)CCNC4=O)n3n2)cn1] benzyl (6R)-6-{[7-bromo-2-(1-ethyl-1H-pyrazol-4-yl) [1,2,4]triazolo[1,5-c]quinazolin-5-yl]amino}-5-oxo- 1,4-diazepane-1-carboxylate LC-MS (Method 2): Rt = 1.24 min; MS (ESIpos): m/z = 604 [M + H]⁺ |
| Inter- mediate 791 | 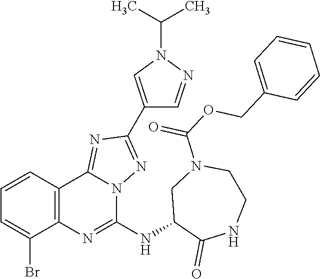 [SMILES: CC(C)n1cc(-c2nc3c4cccc(Br)c4nc(N[C@@H]4CN(C(=O)OCc5ccccc5)CCNC4=O)n3n2)cn1] benzyl (6R)-6-({7-bromo-2-[1-(propan-2-yl)-1H- pyrazol-4-yl][1,2,4]triazolo[1,5-c]quinazolin-5-yl} amino)-5-oxo-1,4-diazepane-1-carboxylate LC-MS (Method 2): Rt = 1.28 min; MS (ESIpos): m/z = 618 [M + H]⁺ |
| Inter- mediate 792 | 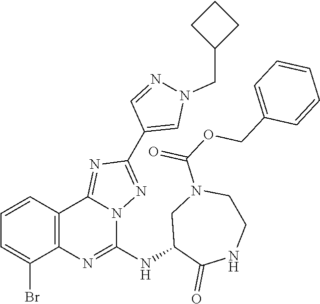 [SMILES: O=C1NCCN(C(=O)OCc2ccccc2)C[C@H]1Nc1nc2c(Br)cccc2c2nc(-c3cnn(CC4CCC4)c3)nn12] benzyl (6R)-6-({7-bromo-2-[1-(cyclobutylmethyl)-1H- pyrazol-4-yl][1,2,4]triazolo[1,5-c]quinazolin-5-yl} amino)-5-oxo-1,4-diazepane-1-carboxylate LC-MS (Method 2): Rt = 1.40 min; MS (ESIpos): m/z = 644 [M + H]⁺ |
| Inter- mediate 793 | 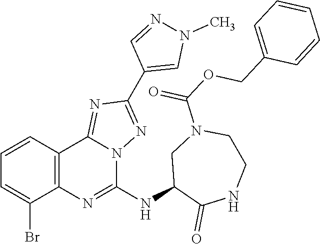 [SMILES: Cn1cc(-c2nc3c4cccc(Br)c4nc(N[C@H]4CN(C(=O)OCc5ccccc5)CCNC4=O)n3n2)cn1] benzyl (6S)-6-{[7-bromo-2-(1-methyl-1H-pyrazol- 4-yl)[1,2,4]triazolo[1,5-c]quinazolin-5-yl] amino}-5-oxo-1,4-diazepane-1-carboxylate LC-MS (Method 2): Rt = 1.17 min; MS (ESIpos): m/z = 590 [M + H]⁺ |
| Inter- mediate 794 | 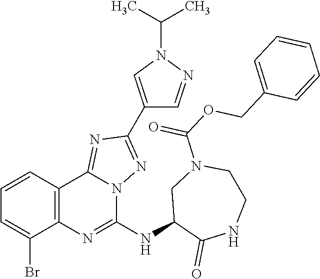 [SMILES: CC(C)n1cc(-c2nc3c4cccc(Br)c4nc(N[C@H]4CN(C(=O)OCc5ccccc5)CCNC4=O)n3n2)cn1] benzyl (6S)-6-({7-bromo-2-[1-(propan-2-yl)-1H- pyrazol-4-yl][1,2,4]triazolo[1,5-c]quinazolin- 5-yl}amino)-5-oxo-1,4-diazepane-1-carboxylate LC-MS (Method 2): Rₜ = 1.32 min; MS (ESIpos): m/z = 618 [M + H]⁺ |
| Inter- mediate 795 | 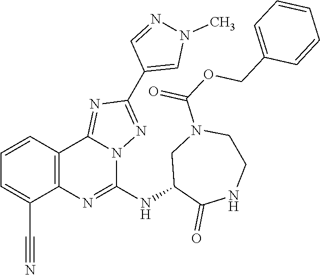 [SMILES: Cn1cc(-c2nc3c4cccc(C#N)c4nc(N[C@@H]4CN(C(=O)OCc5ccccc5)CCNC4=O)n3n2)cn1] benzyl (6R)-6-{[7-cyano-2-(1-methyl-1H-pyrazol-4-yl) [1,2,4]triazolo[1,5-c]quinazolin-5-yl] amino}-5-oxo-1,4-diazepane-1-carboxylate LC-MS (method 1): Rₜ = 1.05 min; MS (ESIpos): m/z = 537 [M + H]⁺ |
| Inter- mediate 796 | 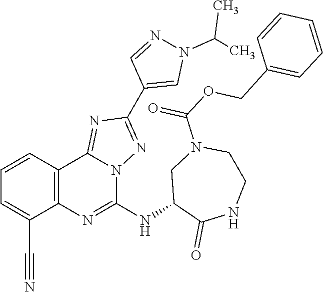 [SMILES: CC(C)n1cc(-c2nc3c4cccc(C#N)c4nc(N[C@@H]4CN(C(=O)OCc5ccccc5)CCNC4=O)n3n2)cn1] benzyl (6R)-6-({7-cyano-2-[1-(propan-2-yl)-1H- pyrazol-4-yl][1,2,4]triazolo[1,5-c]quinazolin- 5-yl}amino)-5-oxo-1,4-diazepane-1-carboxylate LC-MS (method 1): Rₜ = 1.20 min; MS (ESIneg): m/z = 563 [M − H]⁻ |
| Inter- mediate 797 | 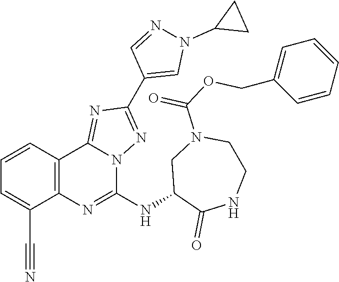 [SMILES: N#Cc1cccc2c1nc(N[C@@H]1CN(C(=O)OCc3ccccc3)CCNC1=O)n1nc(-c3cnn(C4CC4)c3)nc21] benzyl (6R)-6-{[7-cyano-2-(1-cyclopropyl-1H-pyrazol-4- yl)[1,2,4]triazolo[1,5-c]quinazolin-5-yl]amino}- 5-oxo-1,4-diazepane-1-carboxylate LC-MS (method 1): Rₜ = 1.16 min; MS (ESIpos): m/z = 563 [M + H]⁺ |
| Inter- mediate 798 | 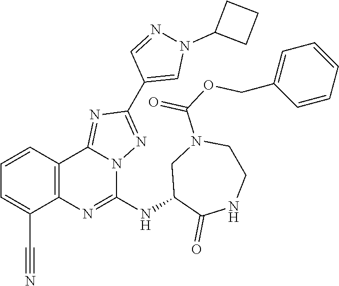 [SMILES: N#Cc1cccc2c1nc(N[C@@H]1CN(C(=O)OCc3ccccc3)CCNC1=O)n1nc(-c3cnn(C4CCC4)c3)nc21] benzyl (6R)-6-{[7-cyano-2-(1-cyclobutyl-1H-pyrazol-4- yl)[1,2,4]triazolo[1,5-c]quinazolin-5-yl] amino}-5-oxo-1,4-diazepane-1-carboxylate LC-MS (method 1): Rₜ = 1.24 min; MS (ESIneg): m/z = 575 [M − H]⁻ |

### Intermediate 799

### benzyl (6R)-6-{[7-(ethanesulfonyl)-2-(4-methoxyphenyl)[1,2,4]triazolo[1,5-c]quinazolin-5-yl]amino}-5-oxo-1,4-diazepane-1-carboxylate

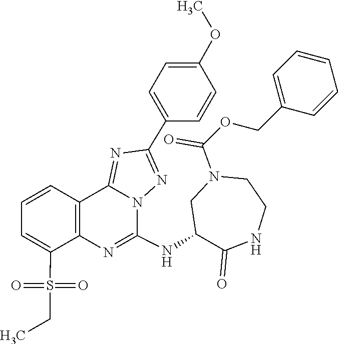 [SMILES: CCS(=O)(=O)c1cccc2c1nc(N[C@@H]1CN(C(=O)OCc3ccccc3)CCNC1=O)n1nc(-c3ccc(OC)cc3)nc21]

Benzyl (6R)-6-{[7-(ethylsulfanyl)-2-(4-methoxyphenyl)[1,2,4]triazolo[1,5-c]quinazolin-5-yl]amino}-5-oxo-1,4-diazepane-1-carboxylate (90.0 mg, 151 μmol) and mCPBA (84.4 mg, 77% purity, 376 μmol) were stirred in dichloromethane (6.2 mL) for 2 h at rt The mixture was diluted with an aqueous sodium thiosulfate solution (10%) and extracted three times with dichloromethane. The combined organic layers were dried over a silicone filter and concentrated under reduced pressure. The crude was dissolved in dichloromethane and extracted with aqueous sodium carbonate solution (2M). The organic phase was dried (silicon filter) and concentrated under reduced pressure to give 84.1 mg (90% purity, 80% yield) of the title compound that was used without further purification.

LC-MS (method 2): Rₜ=1.23 min; MS (ESIpos): m/z=630 [M+H]⁺

The following intermediates were prepared similarly to intermediate 799

| | Structure |
|---|---|
| | IUPAC-Name |
| | LC-MS (method): Retention time; Mass found |
| Example | ¹H-NMR |
| Inter- mediate 800 | 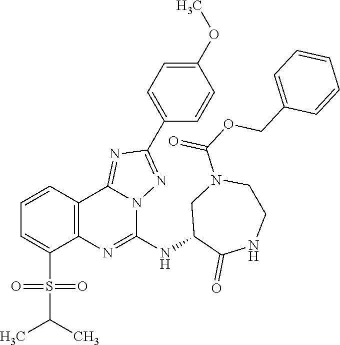 [SMILES: COc1ccc(-c2nc3c4cccc(S(=O)(=O)C(C)C)c4nc(N[C@@H]4CN(C(=O)OCc5ccccc5)CCNC4=O)n3n2)cc1] benzyl (6R)-6-{[2-(4-methoxyphenyl)-7-(propane-2- sulfonyl)[1,2,4]triazolo[1,5-c]quinazolin-5-yl]amino}-5- oxo-1,4-diazepane-1-carboxylate LC-MS (Method 2): Rₜ = 1.25 min; MS (ESIpos): m/z = 644 [M + H]⁺ |
| Inter- mediate 801 | 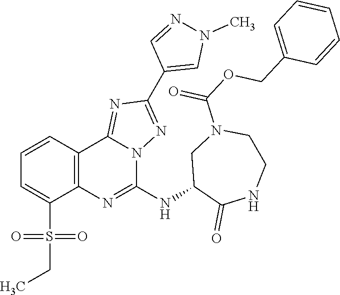 [SMILES: CCS(=O)(=O)c1cccc2c1nc(N[C@@H]1CN(C(=O)OCc3ccccc3)CCNC1=O)n1nc(-c3cnn(C)c3)nc21] benzyl (6R)-6-{[7-(ethanesulfonyl)-2-(1-methyl-1H-pyrazol- 4-yl)[1,2,4]triazolo[1,5-c]quinazolin-5-yl]amino}-5-oxo- 1,4-diazepane-1-carboxylate LC-MS (Method 2): Rₜ = 0.99 min; MS (ESIpos): m/z = 604 [M + H]⁺ |
| Inter- mediate 802 | 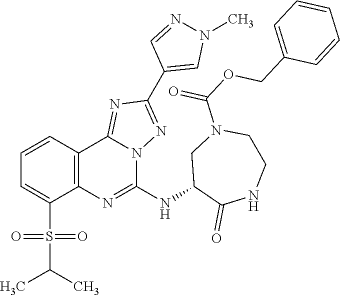 [SMILES: CC(C)S(=O)(=O)c1cccc2c1nc(N[C@@H]1CN(C(=O)OCc3ccccc3)CCNC1=O)n1nc(-c3cnn(C)c3)nc21] benzyl (6R)-6-{[2-(1-methyl-1H-pyrazol-4-yl)-7-(propane- 2-sulfonyl)[1,2,4]triazolo[1,5-c]quinazolin-5-yl]amino}- 5-oxo-1,4-diazepane-1-carboxylate LC-MS (Method 2): Rₜ =1.02 min; MS (ESIpos): m/z = 618 [M + H]⁺ |
| Inter- mediate 803 | 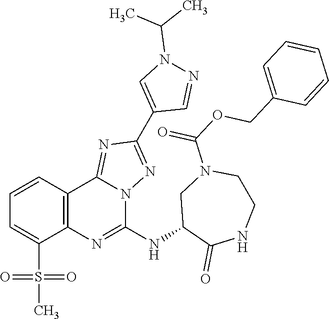 [SMILES: CC(C)n1cc(-c2nc3c4cccc(S(C)(=O)=O)c4nc(N[C@@H]4CN(C(=O)OCc5ccccc5)CCNC4=O)n3n2)cn1] benzyl (6R)-6-({7-(methanesulfonyl)-2-[1-(propan-2-yl)- 1H-pyrazol-4-yl][1,2,4]triazolo[1,5-c]quinazolin-5-yl} amino)-5-oxo-1,4-diazepane-1-carboxylate LC-MS (Method 2): Rₜ = 1.09 min; MS (ESIpos): m/z = 618 [M + H]⁺ |
| Inter- mediate 804 | 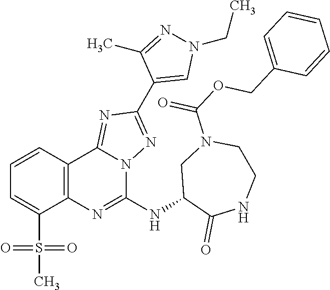 [SMILES: CCn1cc(-c2nc3c4cccc(S(C)(=O)=O)c4nc(N[C@@H]4CN(C(=O)OCc5ccccc5)CCNC4=O)n3n2)c(C)n1] benzyl (6R)-6-{[2-(1-ethyl-3-methyl-1H-pyrazol-4-yl)-7- (methanesulfonyl)[1,2,4]triazolo[1,5-c]quinazolin-5-yl] amino}-5-oxo-1,4-diazepane-1-carboxylate LC-MS (Method 2): Rₜ = 1.08 min; MS (ESIpos): m/z = 618 [M + H]⁺ |

### Intermediate 805

### (3R)-3-{[7-(chloromethyl)-2-(4-methoxyphenyl)[1,2,4]triazolo[1,5-c]quinazolin-5-yl]amino}azepan-2-one

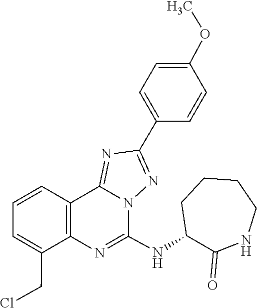 [SMILES: COc1ccc(-c2nc3c4cccc(CCl)c4nc(N[C@@H]4CCCCNC4=O)n3n2)cc1]

(3R)-3-{[7-(Hydroxymethyl)-2-(4-methoxyphenyl)[1,2,4]triazolo[1,5-c]quinazolin-5-yl]amino}azepan-2-one (95.7 mg, 221 μmol) was solubilised in dichloromethane (2 mL) and triethylamine (31 μL, 220 μmol) was added. 4-M ethyl benzene-1-sulfonyl chloride (42.2 mg, 221 μmol) was then added and the mixture was stirred overnight at rt. 4-M ethyl benzene-1-sulfonyl chloride (422 mg, 2.21 mmol) and triethylamine (310 μL, 2.20 mmol) were added again and the mixture was stirred for 48 h at rt. The reaction mixture was quenched with water and extracted with dichloromethane. The combined organic layers were dried (silicone filter) and concentrated under reduced pressure. The crude material was purified by flash column chromatography to give 13 mg (15% yield) of the title compound.

1H-NMR (400 MHz, DMSO-d₆): δ [ppm]=1.21-1.39 (m, 3H), 1.55 (br q, 1H), 1.83-1.93 (m, 1H), 1.93-2.09 (m, 2H), 3.13-3.25 (m, 1H), 3.86 (s, 3H), 4.88 (dd, 1H), 5.11 (d, 1H), 5.35 (d, 1H), 7.15 (d, 2H), 7.45 (t, 1H), 7.76 (d, 1H), 7.89 (dd, 1H), 8.23 (d, 2H), 8.24-8.28 (m, 1H), 8.30 (dd, 1H).

### EXPERIMENTAL SECTION—EXAMPLES

The following examples describe the embodiment of the instant invention, not restricting the invention to these examples only.

### Example 1

### (3S)-3-[(2-phenyl[1,2,4]triazolo[1,5-c]quinazolin-5-yl)amino]azepan-2-one

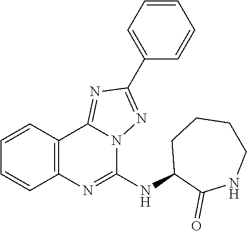 [SMILES: O=C1NCCCC[C@@H]1Nc1nc2ccccc2c2nc(-c3ccccc3)nn12]

5-Chloro-2-phenyl[1,2,4]triazolo[1,5-c]quinazoline (intermediate 3, 75.0 mg, 267 μmol), (3S)-3-aminoazepan-2-one hydrochloride (66.0 mg, 401 μmol) and N,N-diisopropylethylamine (140 μL, 800 μmol) were stirred in DMSO (1.0 mL) for 2 h at 60° C. The reaction mixture was then cooled to rt and diluted with water. The solid was filtered, washed with water and dried under reduced pressure at 60° C. to give 75.3 mg (97% purity, 73% yield) of the title product.

Alternatively, the solid could be purified by preparative HPLC.

LC-MS (method 2): Rₜ=1.32 min; MS (ESIpos): m/z=373 [M+H]⁺

¹H-NMR (400 MHz, DMSO-d6): δ [ppm]=1.26-1.39 (m, 1H), 1.51-1.63 (m, 1H), 1.81-1.96 (m, 2H), 1.98-2.08 (m, 1H), 2.29-2.37 (m, 1H), 3.12-3.22 (m, 1H), 3.35-3.42 (m, 1H), 4.84 (br dd, 1H), 7.43-7.50 (m, 1H), 7.56-7.64 (m, 3H), 7.65-7.70 (m, 1H), 7.72-7.78 (m, 2H), 8.22 (br dd, 1H), 8.27-8.34 (m, 3H).

The following examples were prepared analogously starting from intermediate 16:

| | Structure |
|---|---|
| | IUPAC-Name |
| | LC-MS (method): Retention time; Mass found |
| Example | ¹H-NMR |
| Example 2 | 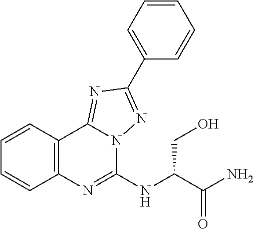 [SMILES: NC(=O)[C@@H](CO)Nc1nc2ccccc2c2nc(-c3ccccc3)nn12] N²-(2-phenyl[1,2,4]triazolo[1,5-c]quinazolin- 5-yl)-D-serinamide LC-MS (Method 2): Rₜ = 1.00 min; MS (ESIpos): m/z = 349 [M + H]⁺ ¹H-NMR (400 MHz, DMSO-d6): δ [ppm] = 3.96 (t, 2H), 4.68-4.76 (m, 1H), 5.19 (t, 1H), 7.32 (s, 1H), 7.41-7.50 (m, 1H), 7.51-7.79 (m, 7H), 8.32 (dt, 3H). |
| Example 3 | 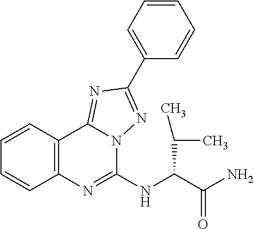 [SMILES: CC(C)[C@@H](Nc1nc2ccccc2c2nc(-c3ccccc3)nn12)C(N)=O] N²-(2-phenyl[1,2,4]triazolo[1,5-c]quinazolin- 5-yl)-D-valinamide LC-MS (Method 2): Rₜ = 1.24 min; MS (ESIneg): m/z = 359 [M − H]⁻ ¹H-NMR (400 MHz, DMSO-d₆): δ [ppm] = 1.01 (d, 3H), 1.04 (d, 3H), 2.25- 2.38 (m, 1H), 4.70 (dd, 1H), 7.26 (d, 1H), 7.38 (s, 1H), 7.46 (ddd, 1H), 7.54- 7.68 (m, 4H), 7.70-7.77 (m, 1H), 7.79 (s, 1H), 8.27-8.37 (m, 3H). |
| Example 4 | 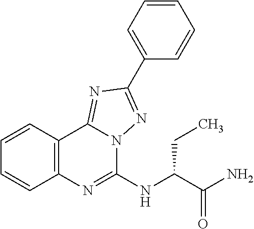 [SMILES: CC[C@@H](Nc1nc2ccccc2c2nc(-c3ccccc3)nn12)C(N)=O] (2R)-2-[(2-phenyl[1,2,4]triazolo[1,5-c] quinazolin-5-yl)amino]butanamide LC-MS (Method 2): Rₜ =1.18 min; MS (ESIpos): m/z = 347 [M + H]⁺ ¹H-NMR (400 MHz, DMSO-d₆): δ [ppm] = 0.96 (t, 3H), 1.85-1.99 (m, 1H), 2.01-2.15 (m, 1H), 4.70 (td, 1H), 7.33 (s, 1H), 7.41-7.50 (m, 1H), 7.53- 7.67 (m, 5H), 7.69-7.78 (m, 2H), 8.24-8.40 (m, 3H). |
| Example 5 | 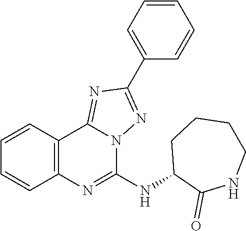 [SMILES: O=C1NCCCC[C@H]1Nc1nc2ccccc2c2nc(-c3ccccc3)nn12] (3R)-3-[(2-phenyl[1,2,4]triazolo[1,5-c] quinazolin-5-yl)amino]azepan-2-one LC-MS (Method 2): Rₜ = 1.31 min; MS (ESIpos): m/z = 373 [M + H]⁺ |

The following examples were prepared analogously to example 1 starting from intermediate 18:

| | Structure |
|---|---|
| | IUPAC-Name |
| | LC-MS (method): Retention time; Mass found |
| Example | ¹H-NMR |
| Example 6 | 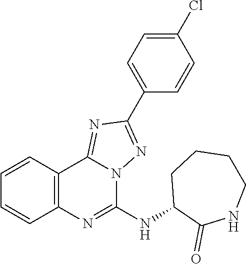 [SMILES: O=C1NCCCC[C@H]1Nc1nc2ccccc2c2nc(-c3ccc(Cl)cc3)nn12] (3R)-3-{[2-(4-chlorophenyl)[1,2,4] [triazolo[1,5-c]quinazolin-5-yl]amino} azepan-2-one LC-MS (Method 2): Rₜ = 1.43 min; MS (ESIpos): m/z = 407 [M + H]⁺ ¹H-NMR (400 MHz, DMSO-d₆): δ [ppm] = 1.26-1.39 (m, 1H), 1.49-1.63 (m, 1H), 1.81-1.95 (m, 2H), 1.98-2.07 (m, 1H), 2.28-2.36 (m, 1H), 3.11-3.22 (m, 1H), 3.40 (br d, 1H), 4.83 (br dd, 1H), 7.43-7.49 (m, 1H), 7.64-7.70 (m, 3H), 7.72-7.78 (m, 2H), 8.19-8.25 (m, 1H), 8.27-8.33 (m, 3H). |
| Example 7 | 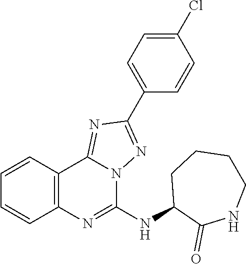 [SMILES: O=C1NCCCC[C@@H]1Nc1nc2ccccc2c2nc(-c3ccc(Cl)cc3)nn12] (3S)-3-{[2-(4-chlorophenyl)[1,2,4] triazolo[1,5-c]quinazolin-5-yl]amino} azepan-2-one LC-MS (Method 2): Rₜ = 1.42 min; MS (ESIneg): m/z = 405 [M − H]⁻ |

The following examples were prepared analogously to example 1 starting from intermediate 20:

| | Structure |
|---|---|
| | IUPAC-Name |
| | LC-MS (method): Retention time; Mass found |
| Example | ¹H-NMR |
| Example 8 | 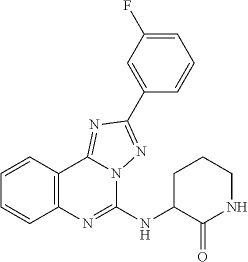 [SMILES: O=C1NCCCC1Nc1nc2ccccc2c2nc(-c3cccc(F)c3)nn12] 3-{[2-(3-fluorophenyl)[1,2,4]triazolo[1,5- c]quinazolin-5-yl]amino}piperidin-2-one LC-MS (Method 2): Rₜ = 1.22 min; MS (ESIpos): m/z = 377 [M + H]⁺ ¹H-NMR (400 MHz, DMSO-d₆): δ [ppm] = 1.84-1.99 (m, 2H), 2.11 (qd, 1H), 2.24-2.31 (m, 1H), 3.18-3.32 (m, 2H), 4.72 (br dd, 1H), 7.36-7.49 (m, 2H), 7.61-7.69 (m, 2H), 7.70-7.77 (m, 1H), 7.85 (br s, 1H), 7.99-8.04 (m, 1H), 8.14 (dt, 1H), 8.30 (dd, 1H), 8.35 (br s, 1H). |
| Example 9 | 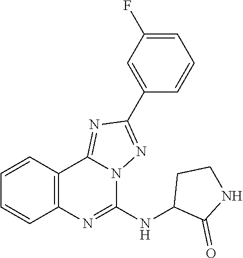 [SMILES: O=C1NCCC1Nc1nc2ccccc2c2nc(-c3cccc(F)c3)nn12] 3-{[2-(3-fluorophenyl)[1,2,4]triazolo[1,5- c]quinazolin-5-yl]amino}pyrrolidin-2-one LC-MS (Method 2): Rₜ = 1.15 min; MS (ESIpos): m/z = 363 [M + H]⁺ ¹H-NMR (400 MHz, DMSO-d₆): δ [ppm] = 4.96 (dt, 1H), 7.37-7.50 (m, 2H), 7.59-7.70 (m, 2H), 7.71-7.79 (m, 1H), 7.98-8.06 (m, 2H), 8.15 (dt, 1H), 8.30 (dd, 1H), 8.36 (d, 1H). |
| Example 10 | 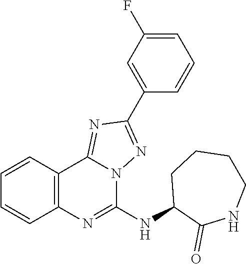 [SMILES: O=C1NCCCC[C@@H]1Nc1nc2ccccc2c2nc(-c3cccc(F)c3)nn12] (3S)-3-{[2-(3-fluorophenyl)[1,2,4]triazolo [1,5-c]quinazolin-5-yl]amino}azepan-2-one LC-MS (Method 2): Rₜ = 1.37 min; MS (ESIpos): m/z = 391 [M + H]⁺ ¹H-NMR (400 MHz, DMSO-d₆): δ [ppm] = 1.11-1.39 (m, 2H), 1.50-1.65 (m, 1H), 1.80-1.95 (m, 2H), 1.98-2.08 (m, 1H), 2.26-2.32 (m, 1H), 3.11-3.23 (m, 1H), 4.84 (dd, 1H), 7.39-7.50 (m, 2H), 7.62-7.71 (m, 2H), 7.72-7.81 (m, 2H), 7.97-8.03 (m, 1H), 8.14 (dt, 1H), 8.21 (dd, 1H), 8.31 (dd, 1H). |
| Example 11 | 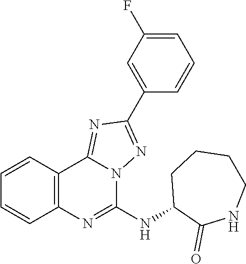 [SMILES: O=C1NCCCC[C@H]1Nc1nc2ccccc2c2nc(-c3cccc(F)c3)nn12] (3R)-3-{[2-(3-fluorophenyl)[1,2,4]triazolo [1,5-c]quinazolin-5-yl]amino}azepan-2-one LC-MS (Method 2): Rₜ = 1.36 min; MS (ESIpos): m/z = 391 [M + H]⁺ |

The following examples were prepared analogously to example 1 starting from intermediate 22:

| | Structure |
|---|---|
| | IUPAC-Name |
| | LC-MS (method): Retention time; Mass found |
| Example | ¹H-NMR |
| Example 12 | 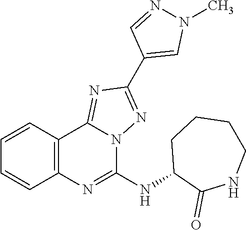 [SMILES: Cn1cc(-c2nc3c4ccccc4nc(N[C@@H]4CCCCNC4=O)n3n2)cn1] (3R)-3-{[2-(1-methyl-1H-pyrazol-4-yl) [1,2,4]triazolo[1,5-c]quinazolin-5- yl]amino}azepan-2-one LC-MS (Method 2): Rₜ = 1.02 min; MS (ESIpos): m/z = 377 [M + H]⁺ ¹H-NMR (400 MHz, DMSO-d₆): δ [ppm] = 1.25-1.41 (m, 1H), 1.47-1.62 (m, 1H), 1.80-1.94 (m, 2H), 1.96-2.08 (m, 1H), 2.27-2.36 (m, 1H), 3.07-3.23 (m, 1H), 3.35-3.42 (m, 1H), 3.95 (s, 3H), 4.82 (dd, 1H), 7.43 (ddd, 1H), 7.60 (d, 1H), 7.63-7.68 (m, 1H), 7.69-7.75 (m, 1H), 8.06 (s, 1H), 8.18-8.26 (m, 2H), 8.49 (s, 1H). |
| Example 13 | 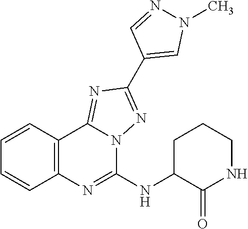 [SMILES: Cn1cc(-c2nc3c4ccccc4nc(NC4CCCNC4=O)n3n2)cn1] 3-{[2-(1-methyl-1H-pyrazol-4-yl)[1,2,4] triazolo[1,5-c]quinazolin-5- yl]amino}piperidin-2-one LC-MS (Method 2): Rₜ = 0.89 min; MS (ESIpos): m/z = 363 [M + H]⁺ ¹H-NMR (400 MHz, DMSO-d₆): δ [ppm] = 1.85-1.99 (m, 2H), 2.01-2.13 (m, 1H), 2.28-2.36 (m, 1H), 3.20-3.29 (m, 2H), 3.96 (s, 3H), 4.66 (dt, 1H), 7.41 (ddd, 1H), 7.60-7.64 (m, 1H), 7.68-7.74 (m, 1H), 7.80 (br s, 1H), 7.98 (d, 1H), 8.05 (d, 1H), 8.23 (dd, 1H), 8.44 (s, 1H). |
| Example 14 | 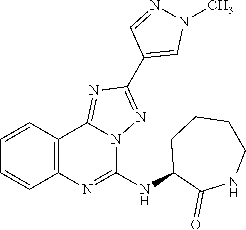 [SMILES: Cn1cc(-c2nc3c4ccccc4nc(N[C@H]4CCCCNC4=O)n3n2)cn1] (3S)-3-{[2-(1-methyl-1H-pyrazol-4-yl) [1,2,4]triazolo[1,5-c]quinazolin-5- yl]amino}azepan-2-one LC-MS (Method 2): Rₜ = 1.02 min; MS (ESIpos): m/z = 377 [M + H]⁺ |
| Example 15 | 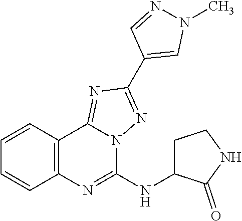 [SMILES: Cn1cc(-c2nc3c4ccccc4nc(NC4CCNC4=O)n3n2)cn1] 3-{[2-(1-methyl-1H-pyrazol-4-yl) [1,2,4]triazolo[1,5-c]quinazolin-5- yl]amino}pyrrolidin-2-one LC-MS (Method 1): Rₜ = 0.81 min; MS (ESIpos): m/z = 349 [M + H]⁺ ¹H-NMR (400 MHz, DMSO-d₆): δ [ppm] = 3.96 (s, 3H), 4.90 (dt, 1H), 7.42 (ddd, 1H), 7.61 (d, 1H), 7.68-7.75 (m, 1H), 7.98 (s, 1H), 8.05 (d, 1H), 8.12 (d, 1H), 8.23 (dd, 1H), 8.43 (s, 1H). |
| Example 16 | 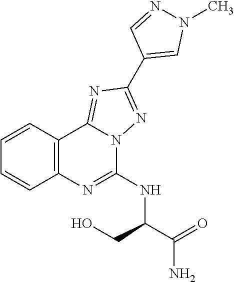 [SMILES: Cn1cc(-c2nc3c4ccccc4nc(N[C@H](CO)C(N)=O)n3n2)cn1] N²-[2-(1-methyl-1H-pyrazol-4-yl)[1,2,4] triazolo[1,5-c]quinazolin-5-yl]-D- serinamide LC-MS (Method 2): Rₜ = 0.71 min; MS (ESIpos): m/z = 353 [M + H]⁺ ¹H-NMR (400 MHz, DMSO-d₆): δ [ppm] = 3.91-3.97 (m, 5H), 4.66-4.74 (m, 1H), 5.18 (t, 1H), 7.31 (s, 1H), 7.35 (d, 1H), 7.40-7.47 (m, 1H), 7.62 (d, 1H), (d, 1H), 7.66-7.75 (m, 2H), 8.08 (d, 1H), 8.24 (dd, 1H), 8.49 (s, 1H). |
| Example 17 | 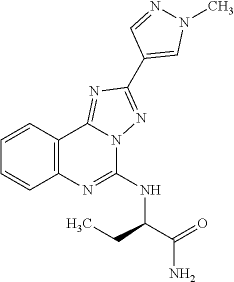 [SMILES: CC[C@@H](Nc1nc2ccccc2c2nc(-c3cnn(C)c3)nn12)C(N)=O] (2R)-2-{[2-(1-methyl-1H-pyrazol-4-yl) [1,2,4]triazolo[1,5-c]quinazolin-5- yl]amino}butanamide LC-MS (Method 2): Rₜ = 0.88 min; MS (ESIpos): m/z = 351 [M + H]⁺ ¹H-NMR (400 MHz, DMSO-d₆): δ [ppm] = 0.94 (t, 3H), 1.92 (dquin, 1H), 2.00- 2.12 (m, 1H), 3.95 (s, 3H), 4.70 (td, 1H), 7.33 (s, 1H), 7.37-7.46 (m, 2H), 7.61 (d, 1H), 7.67-7.76 (m, 2H), 8.08 (d, 1H), 8.23 (dd, 1H), 8.48 (s, 1H). |

### Example 18

### 3-{[2-(1-methyl-1H-pyrazol-4-yl)[1,2,4]triazolo[1,5-c]quinazolin-5-yl]amino}pyrrolidin-2-one, Enantiomer 1

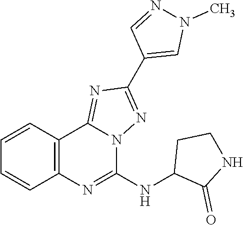 [SMILES: Cn1cc(-c2nc3c4ccccc4nc(NC4CCNC4=O)n3n2)cn1]

Chiral HPLC separation of 3-{[2-(1-methyl-1H-pyrazol-4-yl)[1,2,4]triazolo[1,5-c]quinazolin-5-yl]amino}pyrrolidin-2-one (example 15) was performed (Instrument: Sepiatec: Prep SFC100; Column: Chiralpak IB 5 μm 250×30 mm; Eluent A: CO2, Eluent B: ethanol+0.2 vol-% aqueous ammonia (32%); isocratic: 30% B; flow rate 100.0 mL/min temperature: 40° C.; BPR: 150 bar; MWD @ 254 nm).

Retention time of enantiomer 1: 1.72 min; [α]²⁰_{D}: +11° (c=1) in DMSO.

Instrument: Agilent: 1260, Aurora SFC-Module; column: Chiralpak IB 5 μm 100×4.6 mm; Eluent A: CO2, Eluent B: ethanol+0.2 vol-% aqueous ammonia (32%); isocratic: 30% B; flow rate 4.0 mL/min; temperature: 37.5° C.; BPR: 100 bar; MWD @ 254 nm

### Example 19

### 3-{[2-(1-methyl-1H-pyrazol-4-yl)[1,2,4]triazolo[1,5-c]quinazolin-5-yl]amino}pyrrolidin-2-one, Enantiomer 2

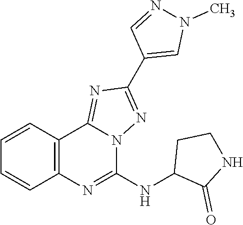 [SMILES: Cn1cc(-c2nc3c4ccccc4nc(NC4CCNC4=O)n3n2)cn1]

The title compound was prepared as described for example 18.

Retention time of enantiomer 2: 2.83 min; [α]²⁰_{D}: −8° (c=1) in DMSO.

### Example 20

### 3-{[2-(1-methyl-1H-pyrazol-4-yl)[1,2,4]triazolo[1,5-c]quinazolin-5-yl]amino}piperidin-2-one, Enantiomer 1

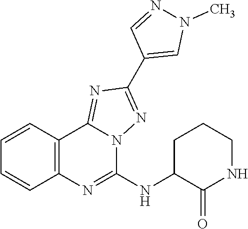 [SMILES: Cn1cc(-c2nc3c4ccccc4nc(NC4CCCNC4=O)n3n2)cn1]

Chiral HPLC separation of 3-{[2-(1-methyl-1H-pyrazol-4-yl)[1,2,4]triazolo[1,5-c]quinazolin-5-yl]amino}piperidin-2-one (example 13) was performed (Instrument: Labomatic HD5000, Labocord-5000; Gilson GX-241, Labcol Vario 4000, column: Chiralpak IA 5μ 250×30 mm; Eluent A: hexane+0.1 vol-% diethylamine (99%); Eluent B: 2-propanol; isocratic: 50% A+50% B; flow rate 40.0 mL/min; UV 254 nm).

Retention time of enantiomer 1: 2.22 min; [α]²⁰_{D}: −33° (c=1) in DMSO.

Instrument: Agilent HPLC 1260; Column: Chiralpak IA 3μ 100×4.6 mm; Eluent A: hexane+0.1 vol-% diethylamine (99%); Eluent B: 2-propanol; isocratic: 50% A+50% B; flow rate 1.4 mL/min; temperature: 25° C.; DAD 254 nm.

### Example 21

### 3-{[2-(1-methyl-1H-pyrazol-4-yl)[1,2,4]triazolo[1,5-c]quinazolin-5-yl]amino}piperidin-2-one, Enantiomer 2

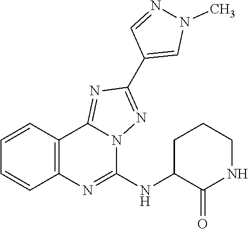 [SMILES: Cn1cc(-c2nc3c4ccccc4nc(NC4CCCNC4=O)n3n2)cn1]

The title compound was prepared as described for example 20.

Retention time of enantiomer 1: 2.22 min; [α]²⁰_{D}: +33° (c=1) in DMSO.

### Example 22

### 6-{[2-(1-methyl-1H-pyrazol-4-yl)[1,2,4]triazolo[1,5-c]quinazolin-5-yl]amino}-1,4-diazepan-5-one, Racemate

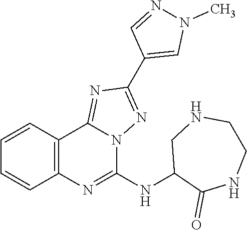 [SMILES: Cn1cc(-c2nc3c4ccccc4nc(NC4CNCCNC4=O)n3n2)cn1]

Benzyl 6-{[2-(1-methyl-1H-pyrazol-4-yl)[1,2,4]triazolo[1,5-c]quinazolin-5-yl]amino}-5-oxo-1,4-diazepane-1-carboxylate (235 mg, 459 μmol) was diluted with DMF (49 mL) and the reaction mixture was purged with argon, Pd/C (48.9 mg, 10% purity, 45.9 μmol) in DMF (1 mL) was added. The reaction mixture was placed under an atmosphere of hydrogen and stirred for 4 h at rt under hydrogen. The reaction mixture was filtered and concentrated under reduced pressure to give 156 mg (94% purity, 85% yield) of the title product without further purification.

LC-MS (method 2): Rₜ=0.75 min; MS (ESIpos): m/z=378 [M+H]⁺

¹H-NMR (400 MHz, DMSO-d6): δ [ppm]=2.52-2.56 (m, 1H), 2.60-2.69 (m, 1H), 3.02 (br dd, 1H), 3.06-3.15 (m, 1H), 3.37-3.48 (m, 2H), 3.95 (s, 3H), 4.87 (ddd, 1H), 7.41-7.47 (m, 1H), 7.56 (d, 1H), 7.65-7.69 (m, 1H), 7.70-7.76 (m, 1H), 8.06 (s, 1H), 8.24 (dd, 1H), 8.30 (dd, 1H), 8.49 (s, 1H).

### Example 23

### 6-{[2-(1-methyl-1H-pyrazol-4-yl)[1,2,4]triazolo[1,5-c]quinazolin-5-yl]amino}-1,4-diazepan-5-one, Enantiomer 1

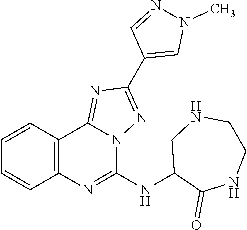 [SMILES: Cn1cc(-c2nc3c4ccccc4nc(NC4CNCCNC4=O)n3n2)cn1]

Benzyl 6-{[2-(1-methyl-1H-pyrazol-4-yl)[1,2,4]triazolo[1,5-c]quinazolin-5-yl]amino}-5-oxo-1,4-diazepane-1-carboxylate, enantiomer 1 (intermediate 167, 64.0 mg, 125 μmol) was diluted with DMF (2.5 mL), and the reaction mixture was purged with argon, Pd/C (13.3 mg) in DMF (1 mL) was added and the reaction mixture was purged 3 times with hydrogen. The reaction was stirred for 4 h at rt under hydrogen. The reaction mixture was filtered and concentrated under reduced pressure to give the title product (29.2 mg, 95% purity, 59% yield).

LC-MS (method 2): Rₜ=0.75 min; MS (ESIpos): m/z=378 [M+H]⁺

¹H-NMR (400 MHz, DMSO-d6): δ [ppm]=2.63-2.72 (m, 1H), 3.04 (br dd, 1H), 3.07-3.15 (m, 1H), 3.37-3.48 (m, 2H), 3.95 (s, 3H), 4.84-4.92 (m, 1H), 7.44 (ddd, 1H), 7.57 (d, 1H), 7.65-7.69 (m, 1H), 7.71-7.77 (m, 1H), 8.06 (d, 1H), 8.25 (dd, 1H), 8.31 (dd, 1H), 8.49 (s, 1H). [α]²⁰_{D}: −56° (c=1) in DMSO.

### Example 24

### 6-{[2-(1-methyl-1H-pyrazol-4-yl)[1,2,4]triazolo[1,5-c]quinazolin-5-yl]amino}-1,4-diazepan-5-one, Enantiomer 2

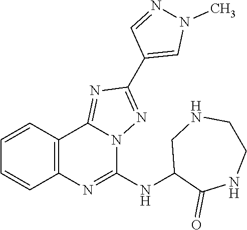 [SMILES: Cn1cc(-c2nc3c4ccccc4nc(NC4CNCCNC4=O)n3n2)cn1]

Benzyl 6-{[2-(1-methyl-1H-pyrazol-4-yl)[1,2,4]triazolo[1,5-c]quinazolin-5-yl]amino}-5-oxo-1,4-diazepane-1-carboxylate, enantiomer 2 (intermediate 168, 63.0 mg, 123 μmol) was diluted with DMF (5.0 mL) and the reaction mixture was purged with argon, Pd/C (13.1 mg) in DMF (1 mL) was added. The reaction mixture was placed under an atmosphere of hydrogen and stirred for 4 h at rt under hydrogen. The reaction mixture was filtered and concentrated under reduced pressure to give the title product (31.8 mg, 95% purity, 65% yield) without further purification.

LC-MS (method 2): Rₜ=0.75 min; MS (ESIpos): m/z=378 [M+H]⁺

¹H-NMR (400 MHz, DMSO-d6): δ [ppm]=2.59 (br t, 1H), 2.70-2.80 (m, 1H), 3.04-3.21 (m, 2H), 3.40-3.52 (m, 2H), 3.95 (s, 3H), 4.89-4.96 (m, 1H), 7.42-7.48 (m, 1H), 7.60 (d, 1H), 7.65-7.70 (m, 1H), 7.71-7.77 (m, 1H), 8.06 (s, 1H), 8.25 (dd, 1H), 8.31-8.37 (m, 1H), 8.49 (s, 1H). [α]²⁰_{D}: +52° (c=1) in DMSO.

The following examples were prepared analogously to example 1 starting from intermediate 24:

| | Structure |
|---|---|
| | IUPAC-Name |
| | LC-MS (method): Retention time; Mass found |
| Example | ¹H-NMR |
| Exam- ple 25 | 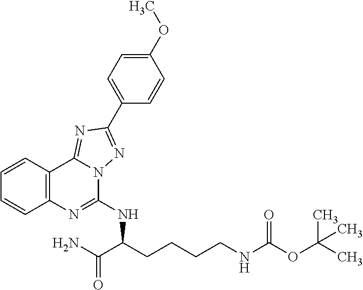 [SMILES: COc1ccc(-c2nc3c4ccccc4nc(N[C@@H](CCCCNC(=O)OC(C)(C)C)C(N)=O)n3n2)cc1] tert-butyl [(5S)-6-amino-5-{[2-(4-methoxyphenyl)[1,2,4] triazolo[1,5-c]quinazolin-5-yl]amino}-6-oxohexyl] carbamate LC-MS (Method 2): Rt = 1.27 min; MS (ESIpos): m/z = 520 [M + H]⁺ ¹H-NMR (400 MHz, DMSO-d₆): δ [ppm] = 1.19-1.48 (m, 13H), 1.85-2.05 (m, 2H), 2.84-2.92 (m, 2H), 3.86 (s, 3H), 4.68-4.77 (m, 1H), 6.76 (br t, 1H), 7.10-7.17 (m, 2H), 7.27 (s, 1H), 7.40-7.46 (m, 1H), 7.57-7.64 (m, 2H), 7.66-7.75 (m, 2H), 8.23-8.31 (m, 3H). |
| Exam- ple 26 | 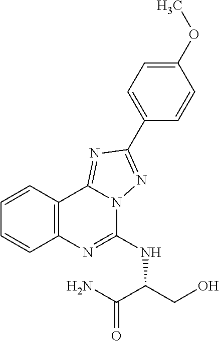 [SMILES: COc1ccc(-c2nc3c4ccccc4nc(N[C@H](CO)C(N)=O)n3n2)cc1] N²-[2-(4-methoxyphenyl)[1,2,4]triazolo [1,5-c]quinazolin-5-yl]-D-serinamide LC-MS (Method 2): Rₜ = 0.98 min; MS (ESIpos): m/z = 379 [M + H]⁺ ¹H-NMR (400 MHz, DMSO-d₆): δ [ppm] = 3.86 (s, 3H), 3.91-3.98 (m, 2H), 4.68-4.75 (m, 1H), 5.19 (br t, 1H), 7.11- 7.18 (m, 2H), 7.32 (s, 1H), 7.41- 7.50 (m, 2H), 7.63 (d, 1H), 7.68 (s, 1H), 7.70-7.76 (m, 1H), 8.22-8.28 (m, 2H), 8.30 (dd, 1H). |
| Exam- ple 27 | 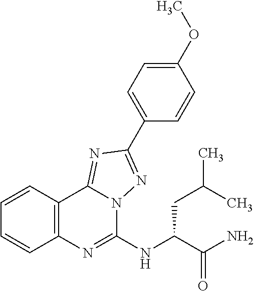 [SMILES: COc1ccc(-c2nc3c4ccccc4nc(N[C@H](CC(C)C)C(N)=O)n3n2)cc1] N²-[2-(4-methoxyphenyl)[1,2,4]triazolo [1,5-c]quinazolin-5-yl]-D-leucinamide LC-MS (Method 2): Rₜ = 1.30 min; MS (ESIneg): m/z = 403 [M − H]⁻ ¹H-NMR (400 MHz, DMSO-d₆): δ [ppm] = 0.95 (dd, 6H), 1.65-1.82 (m, 2H), 1.86-1.97 (m, 1H), 3.86 (s, 3H), 4.73- 4.84 (m, 1H), 7.11-7.17 (m, 2H), 7.19 (s, 1H), 7.39-7.46 (m, 1H), 7.61 (d, 1H), 7.65 (s, 1H), 7.69-7.74 (m, 2H), 8.23-8.31 (m, 3H). |
| Exam- ple 28 | 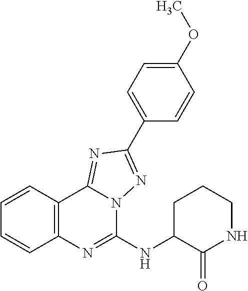 [SMILES: COc1ccc(-c2nc3c4ccccc4nc(NC4CCCNC4=O)n3n2)cc1] 3-{[2-(4-methoxyphenyl)[1,2,4]triazolo[1,5- c]quinazolin-5-yl]amino}piperidin-2-one LC-MS (Method 2): Rₜ = 0.89 min; MS (ESIpos): m/z = 389 [M + H]⁺ ¹H-NMR (400 MHz, DMSO-d₆): δ [ppm] = 1.86-1.97 (m, 2H), 2.09 (qd, 1H), 2.27-2.35 (m, 1H), 3.25 (br d, 2H), 3.86 (s, 3H), 4.70 (dt, 1H), 7.13-7.18 (m, 2H), 7.43 (ddd, 1H), 7.60-7.65 (m, 1H), 7.69-7.74 (m, 1H), 7.80 (br s, 1H), 8.13 (d, 1H), 8.21-8.26 (m, 2H), 8.28 (dd, 1H). |
| Exam- ple 29 | 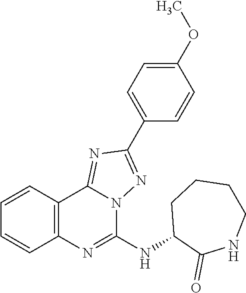 [SMILES: COc1ccc(-c2nc3c4ccccc4nc(N[C@@H]4CCCCNC4=O)n3n2)cc1] (3R)-3-{[2-(4-methoxyphenyl)[1,2,4] triazolo[1,5-c]quinazolin-5- yl]amino}azepan-2-one LC-MS (Method 2): Rₜ = 1.31 min; MS (ESIpos): m/z = 403 [M + H]⁺ ¹H-NMR (400 MHz, DMSO-d₆): δ [ppm] = 1.26-1.40 (m, 1H), 1.50-1.63 (m, 1H), 1.81-1.95 (m, 2H), 2.03 (br d, 1H), 2.28-2.37 (m, 1H), 3.12-3.22 (m, 1H), 3.35 (s, 1H), 3.86 (s, 3H), 4.83 (br dd, 1H), 7.12-7.17 (m, 2H), 7.42-7.49 (m, 1H), 7.64-7.69 (m, 1H), 7.69-7.77 (m, 2H), 8.18-8.25 (m, 3H), 8.29 (dd, 1H). |
| Exam- ple 30 | 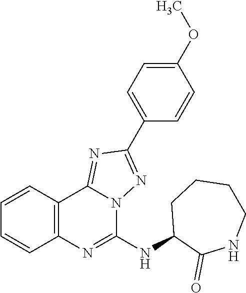 [SMILES: COc1ccc(-c2nc3c4ccccc4nc(N[C@H]4CCCCNC4=O)n3n2)cc1] (3S)-3-{[2-(4-methoxyphenyl)[1,2,4] triazolo[1,5-c]quinazolin-5- yl]amino}azepan-2-one LC-MS (Method 2): Rₜ = 1.30 min; MS (ESIpos): m/z = 403 [M + H]⁺ |
| Exam- ple 31 | 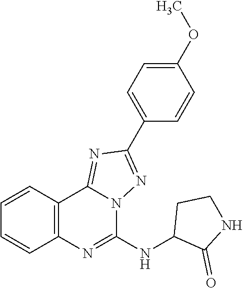 [SMILES: COc1ccc(-c2nc3c4ccccc4nc(NC4CCNC4=O)n3n2)cc1] 3-{[2-(4-methoxyphenyl)[1,2,4] triazolo[1,5-c]quinazolin-5- yl]amino}pyrrolidin-2-one LC-MS (Method 2): Rₜ = 0.84 min; MS (ESIpos): m/z = 375 [M + H]⁺ ¹H-NMR (400 MHz, DMSO-d₆): δ [ppm] = 2.26-2.38 (m, 1H), 3.86 (s, 3H), 4.94 (dt, 1H), 7.13-7.18 (m, 2H), 7.43 (ddd, 1H), 7.59-7.64 (m, 1H), 7.69- 7.75 (m, 1H), 8.00 (s, 1H), 8.22- 8.27 (m, 3H), 8.29 (dd, 1H). |

### Example 32

### 3-{[2-(4-methoxyphenyl)[1,2,4]triazolo[1,5-c]quinazolin-5-yl]amino}azepan-2-one

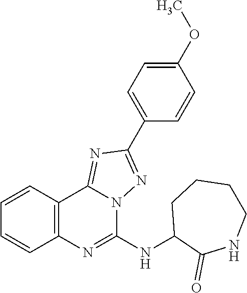 [SMILES: COc1ccc(-c2nc3c4ccccc4nc(NC4CCCCNC4=O)n3n2)cc1]

2-(4-Methoxyphenyl)[1,2,4]triazolo[1,5-c]quinazoline-5(6H)-thione (100 mg, 324 μmol), 3-aminoazepan-2-one (125 mg, 973 μmol) and hydrogen peroxide (400 μL, 33% purity, 4.3 mmol) were stirred in DMSO (3.3 mL) at 80° C. for 4 h. The reaction mixture was then cooled to rt and filtered. The solid was washed with THF to provide the title compound (24.6 mg, 98% purity, 18% yield).

LC-MS (method 2): Rₜ=1.31 min; MS (ESIneg): m/z=401 [M−H]⁻.

¹H-NMR (400 MHz, DMSO-d6): δ [ppm]=1.25-1.40 (m, 1H), 1.50-1.63 (m, 1H), 1.87 (br s, 2H), 1.98-2.07 (m, 1H), 2.33 (br d, 1H), 3.11-3.22 (m, 1H), 3.33 (s, 1H), 3.86 (s, 3H), 4.79-4.87 (m, 1H), 7.15 (d, 2H), 7.45 (s, 1H), 7.64-7.68 (m, 1H), 7.69-7.77 (m, 2H), 8.18-8.25 (m, 3H), 8.29 (dd, 1H).

### Example 33

### N²-[2-(4-methoxyphenyl)[1,2,4]triazolo[1,5-c]quinazolin-5-yl]-L-lysinamide

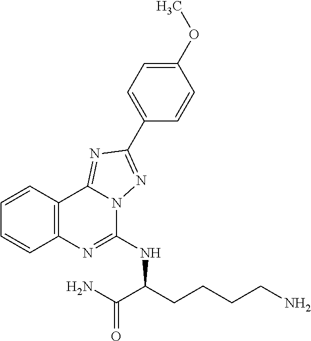 [SMILES: COc1ccc(-c2nc3c4ccccc4nc(N[C@@H](CCCCN)C(N)=O)n3n2)cc1]

Tert-butyl [(5S)-6-amino-5-{[2-(4-methoxyphenyl)[1,2,4]triazolo[1,5-c]quinazolin-5-yl]amino}-6-oxohexyl]carbamate (example 25, 50.0 mg, 96.2 μmol) was solubilised in 1,4-dioxane (1.5 mL) and HCl (400 μL, 4.0 M in dioxane, 1.6 mmol) was added. The mixture was stirred overnight at rt. The mixture was then basified with sat. sodium hydrogen carbonate (pH 10), the organic solvent was evaporated and the suspension was filtered, washed with water and the solid dried under reduced pressure at 60° C. to give the title compound (36.0 mg, 90% purity, 80% yield).

LC-MS (method 2): Rₜ=1.13 min; MS (ESIpos): m/z=420 [M+H]⁺

¹H-NMR (400 MHz, DMSO-d6): δ [ppm]=1.40 (br s, 4H), 1.85-2.05 (m, 2H), 3.86 (s, 3H), 4.73 (dd, 1H), 7.12-7.17 (m, 2H), 7.27 (s, 1H), 7.41-7.47 (m, 1H), 7.62 (d, 1H), 7.67-7.75 (m, 2H), 8.23-8.32 (m, 3H).

### Example 34

### 6-{[2-(4-methoxyphenyl)[1,2,4]triazolo[1,5-c]quinazolin-5-yl]amino}-1,4-diazepan-5-one

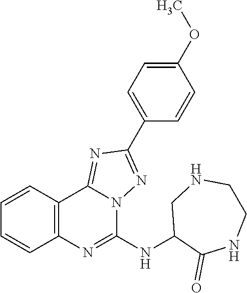 [SMILES: COc1ccc(-c2nc3c4ccccc4nc(NC4CNCCNC4=O)n3n2)cc1]

Benzyl 6-{[2-(4-methoxyphenyl)[1,2,4]triazolo[1,5-c]quinazolin-5-yl]amino}-5-oxo-1,4-diazepane-1-carboxylate (example 27) (788 mg, 1.47 mmol) was solubilized in DMF (50 mL) and the mixture was placed under argon. Pd/C (156 mg) in DMF (1 mL) was added. The reaction was placed under an atmosphere of hydrogen and it was stirred for 2 h at rt. The mixture was filtered and concentrated under reduced pressure to give the title product (719 mg, 97% purity, 118% yield) without further purification.

LC-MS (method 2): Rₜ=1.04 min; MS (ESIpos): m/z=404 [M+H]⁺

¹H-NMR (400 MHz, DMSO-d₆): δ [ppm]=3.00-3.18 (m, 2H), 3.43 (br d, 2H), 3.86 (s, 3H), 4.86-4.94 (m, 1H), 7.11-7.18 (m, 2H), 7.46 (td, 1H), 7.65-7.71 (m, 2H), 7.72-7.78 (m, 1H), 8.19-8.25 (m, 2H), 8.30 (br dd, 2H).

### Example 35

### 6-{[2-(4-methoxyphenyl)[1,2,4]triazolo[1,5-c]quinazolin-5-yl]amino}-1,4-diazepan-5-one, Enantiomer 1

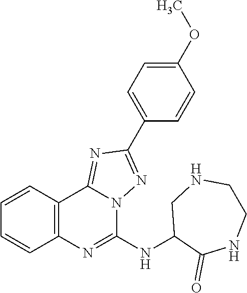 [SMILES: COc1ccc(-c2nc3c4ccccc4nc(NC4CNCCNC4=O)n3n2)cc1]

Chiral HPLC separation of 6-{[2-(4-methoxyphenyl)[1,2,4]triazolo[1,5-c]quinazolin-5-yl]amino}-1,4-diazepan-5-one (example 34) was performed (Instrument: Labomatic HD5000, Labocord-5000; Gilson GX-241, Labcol Vario 4000, Column: Chiralpak ID 5μ 250×30 mm; Eluent: tert.-butylmethylether+0.1 vol-% diethylamine (99%)/ethanol 90:10; flow rate 50.0 mL/min; UV 254 nm).

Retention time of enantiomer 1: 5.73 min; [α]²⁰_{D}: +62° (c=1) in DMSO.

Instrument: Agilent HPLC 1260; Column: Chiralpak ID 3μ 100×4.6 mm; Eluent: tert.-butylmethylether+0.1 vol-% diethylamine (99%)/ethanol 90:10; flow rate 1.4 mL/min; temperature: 25° C.; DAD 254 nm.

### Example 36

### 6-{[2-(4-methoxyphenyl)[1,2,4]triazolo[1,5-c]quinazolin-5-yl]amino}-1,4-diazepan-5-one, Enantiomer 2

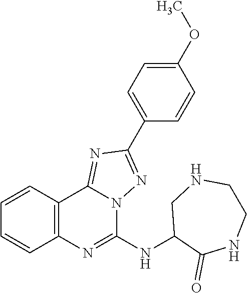 [SMILES: COc1ccc(-c2nc3c4ccccc4nc(NC4CNCCNC4=O)n3n2)cc1]

The title compound was prepared as described for example 35.

Retention time of enantiomer 2: 5.73 min; [α]²⁰_{D}: −72° (c=1) in DMSO.

### Example 37

### 3-{[2-(4-methoxyphenyl)[1,2,4]triazolo[1,5-c]quinazolin-5-yl]amino}piperidin-2-one, Enantiomer 1

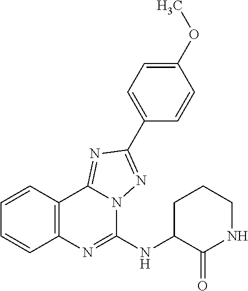 [SMILES: COc1ccc(-c2nc3c4ccccc4nc(NC4CCCNC4=O)n3n2)cc1]

Chiral HPLC separation of 3-{[2-(4-methoxyphenyl)[1,2,4]triazolo[1,5-c]quinazolin-5-yl]amino}piperidin-2-one (example 28) was performed (Instrument: Sepiatec: Prep SFC100; Column: Chiralpak ID 5 μm 250×30 mm; Eluent A: CO2, Eluent B: ethanol+0.2 vol-% aqueous ammonia (32%); isocratic: 49% B; flow rate 100.0 mL/min temperature: 40° C.; BPR: 150 bar; MWD @ 254 nm).

Retention time of enantiomer 1: 1.73 min; [α]²⁰_{D}: +27° (c=1) in DMSO.

Instrument: Agilent: 1260, Aurora SFC-Modul; Column: Chiralpak ID 5 μm 100×4.6 mm; Eluent A: CO2, Eluent B: ethanol+0.2 vol-% aqueous ammonia (32%); isocratic: 49% B; flow rate 4.0 mL/min; temperature: 37.5° C.; BPR: 100 bar; MWD @ 254 nm.

### Example 38

### 3-{[2-(4-methoxyphenyl)[1,2,4]triazolo[1,5-c]quinazolin-5-yl]amino}piperidin-2-one, Enantiomer 2

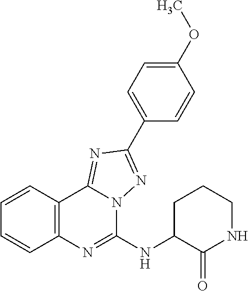 [SMILES: COc1ccc(-c2nc3c4ccccc4nc(NC4CCCNC4=O)n3n2)cc1]

The title compound was prepared as described for example 39.

Retention time of enantiomer 2: 2.89 min; [α]²⁰_{D}: −29° (c=1) in DMSO.

### Example 39

### (3R)-3-{[2-(4-methoxyphenyl)[1,2,4]triazolo[1,5-c]quinazolin-5-yl]amino}pyrrolidin-2-one

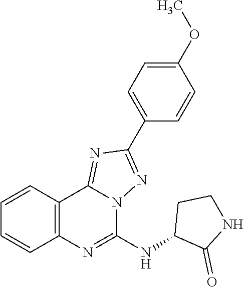 [SMILES: COc1ccc(-c2nc3c4ccccc4nc(N[C@@H]4CCNC4=O)n3n2)cc1]

Chiral HPLC separation of example 31 was performed (Instrument: Labomatic HD5000, Labocord-5000; Gilson GX-241, Labcol Vario 4000, Column: YMC Cellulose SB 5μ 250×30 mm; Eluent: tert.-butylmethylether+0.1 vol-% diethylamine (99%)/ethanol 90:10; flow rate 40.0 mL/min; UV 254 nm).

Retention time of enantiomer 1: 2.78 min; [α]²⁰_{D}: +8° (c=1) in DMSO.

Instrument: Agilent HPLC 1260; Column: YMC Cellulose SB 3μ 100×4.6 mm; Eluent: tert.-butylmethylether+0.1 vol-% diethylamine (99%)/ethanol 90:10; flow rate 1.4 mL/min; temperature: 25° C.; DAD 254 nm.

Alternatively, example 39 could be prepared analogously to example 1 starting from intermediate 24 and enantiopure (3R)-3-aminopyrrolidin-2-one.

### Example 40

### (3S)-3-{[2-(4-methoxyphenyl)[1,2,4]triazolo[1,5-c]quinazolin-5-yl]amino}pyrrolidin-2-one

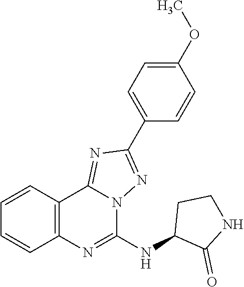 [SMILES: COc1ccc(-c2nc3c4ccccc4nc(N[C@H]4CCNC4=O)n3n2)cc1]

Chiral HPLC separation of example 31 was performed (Instrument: Labomatic HD5000, Labocord-5000; Gilson GX-241, Labcol Vario 4000, Column: YMC Cellulose SB 5μ 250×30 mm; Eluent: tert.-butylmethylether+0.1 vol-% diethylamine (99%)/ethanol 90:10; flow rate 40.0 mL/min; UVS24 nm).

Retention time of enantiomer 1: 3.81 min; [α]²⁰_{D}: −5° (c=1) in DMSO.

Instrument: Agilent HPLC 1260; Column: YMC Cellulose SB 3μ 100×4.6 mm; Eluent: tert.-butyl methyl ether+0.1 vol-% diethylamine (99%)/ethanol 90:10; flow rate 1.4 mL/min; temperature: 25° C.; DAD 254 nm.

The following examples were prepared analogously to example 1 starting from intermediate 27:

| | Structure |
|---|---|
| | IUPAC-Name |
| | LC-MS (method): Retention time; Mass found |
| Example | ¹H-NMR |
| Example 41 | 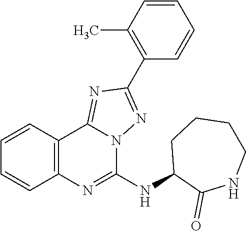 [SMILES: Cc1ccccc1-c1nc2c3ccccc3nc(N[C@H]3CCCCNC3=O)n2n1] (3S)-3-{[2-(2-methylphenyl)[1,2,4] triazolo[1,5-c]quinazolin-5- yl]amino}azepan-2-one LC-MS (Method 2): Rₜ = 0.90 min; MS (ESIpos): m/z = 387 [M + H]⁺ ¹H-NMR (400 MHz, DMSO-d₆): δ [ppm] = 1.22-1.38 (m, 1H), 1.50-1.62 (m, 1H), 1.81-1.97 (m, 2H), 1.98-2.08 (m, 1H), 2.34-2.43 (m, 1H), 2.73 (s, 3H), 3.11-3.22 (m, 1H), 3.34-3.41 (m, 1H), 4.83 (dd, 1H), 7.37-7.49 (m, 4H), 7.67-7.71 (m, 1H), 7.72-7.79 (m, 2H), 8.12-8.17 (m, 1H), 8.20 (dd, 1H), 8.30 (dd, 1H). |
| Example 42 | 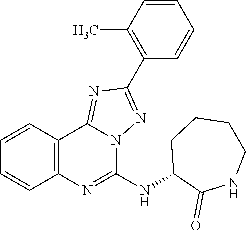 [SMILES: Cc1ccccc1-c1nc2c3ccccc3nc(N[C@@H]3CCCCNC3=O)n2n1] (3R)-3-{[2-(2-methylphenyl)[1,2,4] triazolo[1,5-c]quinazolin-5- yl]amino}azepan-2-one LC-MS (Method 2): Rₜ = 0.89 min; MS (ESIpos): m/z = 387 [M + H]⁺ |
| Example 43 | 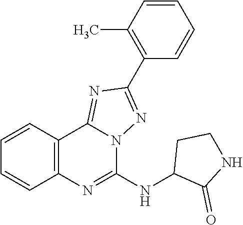 [SMILES: Cc1ccccc1-c1nc2c3ccccc3nc(NC3CCNC3=O)n2n1] 3-{[2-(2-methylphenyl)[1,2,4] triazolo[1,5-c]quinazolin-5- yl]amino}pyrrolidin-2-one LC-MS (Method 2): Rₜ = 0.90 min; MS (ESIpos): m/z = 359 [M + H]⁺ ¹H-NMR (400 MHz, DMSO-d₆): δ [ppm] = 2.34-2.44 (m, 1H), 2.75 (s, 3H), 3.35 (br d, 1H), 4.95 (dt, 1H), 7.36- 7.48 (m, 4H), 7.61-7.66 (m, 1H), 7.70- 7.77 (m, 1H), 8.00 (s, 1H), 8.15 (d, 1H), 8.26 (d, 1H), 8.30 (dd, 1H). |
| Example 44 | 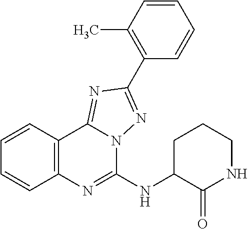 [SMILES: Cc1ccccc1-c1nc2c3ccccc3nc(NC3CCCNC3=O)n2n1] 3-{[2-(2-methylphenyl)[1,2,4] triazolo[1,5-c]quinazolin-5- yl]amino}piperidin-2-one LC-MS (Method 2): Rₜ = 0.92 min; MS (ESIpos): m/z = 373 [M + H]⁺ ¹H-NMR (400 MHz, DMSO-d₆): δ [ppm] = 1.84-1.97 (m, 2H), 2.11 (qd, 1H), 2.29 (br d, 1H), 2.75 (s, 3H), 3.19-3.30 (m, 2H), 4.70 (dt, 1H), 7.36-7.48 (m, 4H), 7.62-7.67 (m, 1H), 7.70-7.75 (m, 1H), 7.81 (br s, 1H), 8.12-8.18 (m, 2H), 8.29 (dd, 1H). |

### Example 45

### 6-{[2-(2-methylphenyl)[1,2,4]triazolo[1,5-c]quinazolin-5-yl]amino}-1,4-diazepan-5-one

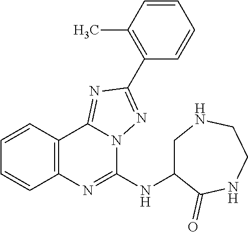 [SMILES: Cc1ccccc1-c1nc2c3ccccc3nc(NC3CNCCNC3=O)n2n1]

Benzyl 6-{[2-(2-methylphenyl)[1,2,4]triazolo[1,5-c]quinazolin-5-yl]amino}-5-oxo-1,4-diazepane-1-carboxylate (example 170) (245 mg, 470 μmol) was solubilized in DMF (51 mL) and the reaction was placed under argon. Pd/C (50.0 mg) in DMF (1 mL) was added and the reaction mixture was placed under an atmosphere of hydrogen. The reaction was stirred for 2 h at rt and then filtered and concentrated under reduced pressure to provide the title compound (300 mg, 94% purity, 155% yield) without further purification.

LC-MS (method 2): Rₜ=1.12 min; MS (ESIpos): m/z=388 [M+H]⁺

¹H-NMR (400 MHz, DMSO-d6): δ [ppm]=2.65-2.72 (m, 2H), 3.00-3.07 (m, 1H), 3.07-3.17 (m, 1H), 3.39-3.48 (m, 2H), 4.85-4.92 (m, 1H), 7.37-7.50 (m, 4H), 7.68-7.79 (m, 3H), 8.14 (d, 1H), 8.27-8.33 (m, 2H).

### Example 46

### 6-{[2-(2-methylphenyl)[1,2,4]triazolo[1,5-c]quinazolin-5-yl]amino}-1,4-diazepan-5-one, Enantiomer 1

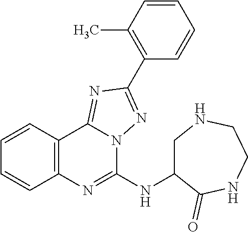 [SMILES: Cc1ccccc1-c1nc2c3ccccc3nc(NC3CNCCNC3=O)n2n1]

Chiral HPLC separation of 6-{[2-(2-methylphenyl)[1,2,4]triazolo[1,5-c]quinazolin-5-yl]amino}-1,4-diazepan-5-one (example 45) was performed (Instrument: Labomatic HD5000, Labocord-5000; Gilson GX-241, Labcol Vario 4000, Column: Reprosil Chiral NR 8μ 250×30 mm; Eluent: methanol+0.1 vol-% diethylamine (99%)/ethanol 50:50%; flow rate 40.0 mL/min; UV 254 nm).

Retention time of enantiomer 1: 2.95 min; [α]²⁰_{D}: +71° (c=1) in DMSO.

Instrument: Agilent HPLC 1260; Säule: Reprosil Chiral NR 8μ 100×4.6 mm; Eluent: methanol+0.1 vol-% diethylamine (99%)/ethanol 50:50; flow rate 1.4 mL/min; temperature: 25° C.; DAD 254 nm

### Example 47

### 6-{[2-(2-methylphenyl)[1,2,4]triazolo[1,5-c]quinazolin-5-yl]amino}-1,4-diazepan-5-one, Enantiomer 2

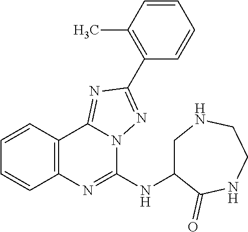 [SMILES: Cc1ccccc1-c1nc2c3ccccc3nc(NC3CNCCNC3=O)n2n1]

The title compound was prepared as described for example 46.

Retention time of enantiomer 2: 4.56 min; [α]²⁰_{D}: −70° (c=1) in DMSO.

### Example 48

### 3-{[2-(2-methylphenyl)[1,2,4]triazolo[1,5-c]quinazolin-5-yl]amino}piperidin-2-one, enantiomer 1

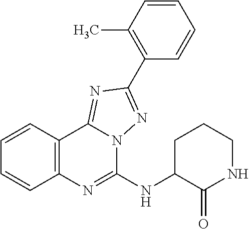 [SMILES: Cc1ccccc1-c1nc2c3ccccc3nc(NC3CCCNC3=O)n2n1]

Chiral HPLC separation of 3-{[2-(2-methylphenyl)[1,2,4]triazolo[1,5-c]quinazolin-5-yl]amino}piperidin-2-one (example 44) was performed (Instrument: Sepiatec: Prep SFC100; Column: Chiralpak IG 5 μm 250×30 mm; Eluent A: CO2, Eluent B: ethanol+0.2 vol-% aqueous ammonia (32%); isocratic: 45% B; flow rate 100.0 mL/min temperature: 40° C.; BPR: 150 bar; MWD @ 254 nm).

Retention time of enantiomer 1: 1.70 min; [α]²⁰_{D}: −24° (c=1) in DMSO.

Instrument: Agilent: 1260, Aurora SFC-Module; Column: Chiralpak IG 5 μm 100×4.6 mm; Eluent A: CO2, Eluent B: ethanol+0.2 vol-% aqueous ammonia (32%); isocratic: 45% B; flow rate 4.0 mL/min; temperature: 37.5° C.; BPR: 100 bar; MWD @ 254 nm.

### Example 49

### 3-{[2-(2-methylphenyl)[1,2,4]triazolo[1,5-c]quinazolin-5-yl]amino}piperidin-2-one, Enantiomer 2

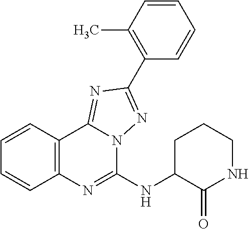 [SMILES: Cc1ccccc1-c1nc2c3ccccc3nc(NC3CCCNC3=O)n2n1]

The title compound was prepared as described for example 48.

Retention time of enantiomer 2: 1.70 min; [α]²⁰_{D}: −27° (c=1) in DMSO.

### Example 50

### 3-{[2-(2-methylphenyl)[1,2,4]triazolo[1,5-c]quinazolin-5-yl]amino}pyrrolidin-2-one, Enantiomer 1

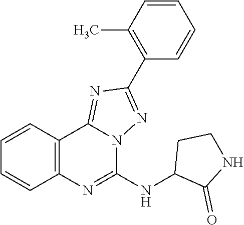 [SMILES: Cc1ccccc1-c1nc2c3ccccc3nc(NC3CCNC3=O)n2n1]

Chiral HPLC separation of 3-{[2-(2-methylphenyl)[1,2,4]triazolo[1,5-c]quinazolin-5-yl]amino}pyrrolidin-2-one (example 43) was performed (Instrument: Sepiatec: Prep SFC100; Column: Chiralpak IG 5 μm 250×30 mm; Eluent A: CO2, Eluent B: ethanol+0.2 vol-% aqueous ammonia (32%); isocratic: 45% B; flow rate 100.0 mL/min Temperature: 40° C.; BPR: 150 bar; MWD @ 254 nm).

Retention time of enantiomer 1: 1.58 min; [α]²⁰_{D}: +17 (c=1) in DMSO.

Instrument: Agilent: 1260, Aurora SFC-Modul; column: Chiralpak IG 5 μm 100×4.6 mm; Eluent A: CO2, Eluent B: ethanol+0.2 vol-% aqueous ammonia (32%); isocratic: 45% B; flow rate 4.0 mL/min; temperature: 37.5° C.; BPR: 100 bar; MWD @ 254 nm.

### Example 51

### 3-{[2-(2-methylphenyl)[1,2,4]triazolo[1,5-c]quinazolin-5-yl]amino}pyrrolidin-2-one, enantiomer 2

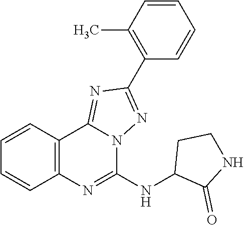 [SMILES: Cc1ccccc1-c1nc2c3ccccc3nc(NC3CCNC3=O)n2n1]

The title compound was prepared as described for example 50

Retention time of enantiomer 2: 2.52 min; [α]²⁰_{D}: −13° (c=1) in DMSO.

The following examples were prepared analogously to example 1 starting from the corresponding intermediates:

| | Structure |
|---|---|
| | IUPAC-Name |
| | LC-MS (method): Retention time; Mass found |
| Example | ¹H-NMR |
| Example 52 | 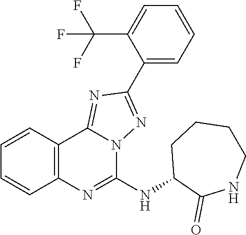 [SMILES: O=C1NCCCC[C@H]1Nc1nc2ccccc2c2nc(-c3ccccc3C(F)(F)F)nn12] (3R)-3-({2-[2-(trifluoromethyl)phenyl] [1,2,4]triazolo[1,5-c]quinazolin-5- yl}amino)azepan-2-one LC-MS (Method 2): Rt = 1.35 min; MS (ESIpos): m/z = 441 [M + H]⁺ ¹H-NMR (400 MHz, DMSO-d₆): δ [ppm] = 1.26-1.40 (m, 1H), 1.51-1.65 (m, 1H), 1.80-1.95 (m, 2H), 1.98-2.10 (m, 1H), 2.30-2.37 (m, 1H), 3.12-3.23 (m, 1H), 3.36-3.43 (m, 1H), 4.85 (dd, 1H), 7.47 (ddd, 1H), 7.67-7.71 (m, 1H), 7.74-7.78 (m, 1H), 7.79 (d, 1H), 7.98 (d, 2H), 8.23 (dd, 1H), 8.32 (dd, 1H), 8.50 (d, 2H). |
| Example 53 | 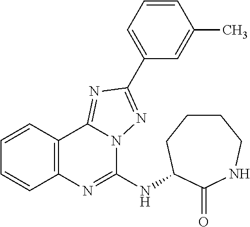 [SMILES: Cc1cccc(-c2nc3c4ccccc4nc(N[C@@H]4CCCCNC4=O)n3n2)c1] (3R)-3-{[2-(3-methylphenyl)[1,2,4] triazolo[1,5-c]quinazolin-5- yl]amino}azepan-2-one LC-MS (Method 3): Rt = 2.68 min., MS (ESIpos): m/z = 387[M + H]⁺. ¹H NMR (400 MHz, DMSO-d6): δ [ppm] = 1.23-1.36 (m, 1H), 1.48-1.61 (m, 1H), 1.78-2.04 (m, 3H), 2.25-2.33 (m, 1H), 2.42 (s, 3H), 3.09-3.18 (m, 1H), 3.31-3.38 (m, 1H), 4.78-4.84 (m, 1H), 7.33-7.37 (m, 1H), 7.40-7.48 (m, 2H), 7.62-7.66 (m, 1H), 7.68-7.74 (m, 2H), 8.04-8.10 (m, 2H), 8.15-8.20 (m, 1H), 8.26-8.29 (m, 1H). |
| Example 54 | 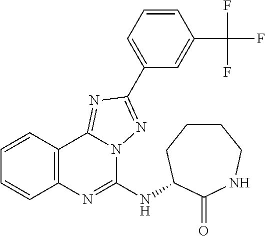 [SMILES: O=C1NCCCC[C@H]1Nc1nc2ccccc2c2nc(-c3cccc(C(F)(F)F)c3)nn12] (3R)-3-({2-[3-(trifluoromethyl)phenyl] [1,2,4]triazolo[1,5-c]quinazolin-5- yl}amino)azepan-2-one LC-MS (Method 3): Rt = 2.92 min., MS (ESIpos): m/z = 441(M + H)⁺. ¹H NMR (400 MHz, DMSO-d6): δ [ppm] = 1.23-1.36 (m, 1H), 1.50-1.62 (m, 1H), 1.78-2.04 (m, 3H), 2.25-2.32 (m, 1H), 3.08-3.39 (m, 2H), 4.82 (dd, 1H), 7.41-7.46 (m, 1H), 7.63-7.66 (m, 1H), 7.70-7.75 (m, 1H), 7.78 (d, 1H), 7.83 (t, 1H), 7.93 (d, 1H), 8.13-8.19 (m, 1H), 8.31 (dd, 1H), 8.50 (s, 1H), 8.55 (d, 1H). |
| Example 55 | 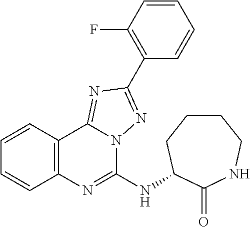 [SMILES: O=C1NCCCC[C@H]1Nc1nc2ccccc2c2nc(-c3ccccc3F)nn12] (3R)-3-{[2-(2-fluorophenyl)[1,2,4] triazolo[1,5-c]quinazolin-5- yl]amino}azepan-2-one LC-MS (Method 3): Rt = 2.41 min., MS (ESIpos): m/z = 391[M + H]⁺. ¹H NMR (400 MHz, DMSO-d6): δ [ppm] = 1.22-1.36 (m, 1H), 1.46-1.59 (m, 1H), 1.77-1.92 (m, 2H), 1.94-2.04 (m, 1H), 2.31 (d, 1H), 3.08-3.18 (m, 1H), 3.24-3.39 (m, 1H), 4.80 (dd,1H), 7.37-7.46 (m, 3H), 7.56-7.75 (m, 4H), 8.15- 8.29 (m, 3H). |
| Example 56 | 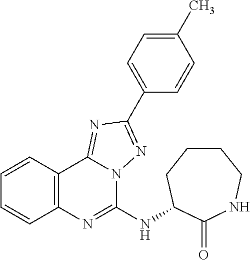 [SMILES: Cc1ccc(-c2nc3c4ccccc4nc(N[C@@H]4CCCCNC4=O)n3n2)cc1] (3R)-3-{[2-(4-methylphenyl)[1,2,4] triazolo[1,5-c]quinazolin-5- yl]amino}azepan-2-one LC-MS (Method 3): Rt = 2.66 min., MS (ESIpos): m/z = 387 [M + H]⁺. ¹H NMR (400 MHz, methanol-d4): δ [ppm] = 1.41-1.55 (m, 1H), 1.65-1.77 (m, 1H), 1.89-2.16 (m, 3H), 2.36-2.44 (m, 4H), 3.21-3.36 (m, 1H + CD₃OD), 3.40- 3.50 (m, 1H), 5.01 (d, 1H), 7.34 (d, 2H), 7.40-7.45 (m, 1H), 7.68-7.72 (m, 2H), 8.18 (d, 2H), 8.34-8.39 (m, 1H) |
| Example 57 | 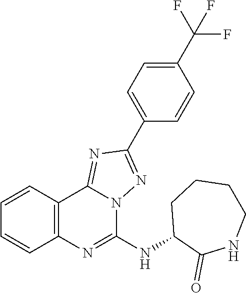 [SMILES: O=C1NCCCC[C@H]1Nc1nc2ccccc2c2nc(-c3ccc(C(F)(F)F)cc3)nn12] (3R)-3-({2-[4-(trifluoromethyl)phenyl] [1,2,4]triazolo[1,5-c]quinazolin-5- yl}amino)azepan-2-one LC-MS (Method 2): Rt = 1.48 min; MS (ESIpos): m/z = 441 [M + H]⁺ ¹H-NMR (400 MHz, DMSO-d₆): δ [ppm] = 1.14-1.41 (m, 2H), 1.51-1.65 (m, 1H), 1.80-1.95 (m, 2H), 1.98-2.10 (m, 1H), 2.30-2.37 (m, 1H), 3.12-3.23 (m, 1H), 4.85 (dd, 1H), 7.47 (ddd, 1H), 7.67-7.71 (m, 1H), 7.74-7.78 (m, 1H), 7.79 (d, 1H), 7.98 (d, 2H), 8.23 (dd, 1H), 8.32 (dd, 1H), 8.50 (d, 2H). |
| Example 58 | 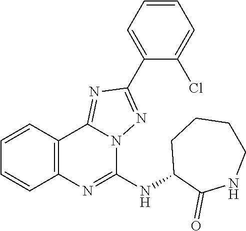 [SMILES: O=C1NCCCC[C@H]1Nc1nc2ccccc2c2nc(-c3ccccc3Cl)nn12] (3R)-3-{[2-(2-chlorophenyl)[1,2,4] triazolo[1,5-c]quinazolin-5- yl]amino}azepan-2-one LC-MS (Method 2): Rt = 1.47 min; MS (ESIpos): m/z = 407 [M + H]⁺ ¹H-NMR (400 MHz, DMSO-d₆): δ [ppm] = 1.27-1.40 (m, 1H), 1.51-1.66 (m, 1H), 1.81-1.95 (m, 2H), 1.97-2.09 (m, 1H), 2.30 (dd, 1H), 3.11-3.23 (m, 1H), 3.37-3.44 (m, 1H), 4.79-4.90 (m, 1H), 7.44-7.50 (m, 1H), 7.63-7.67 (m, 2H), 7.67-7.71 (m, 1H), 7.75 (br d, 1H), 7.77-7.81 (m, 1H), 8.18-8.28 (m, 3H), 8.32 (br d, 1H). |
| Example 59 | 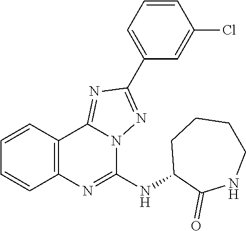 [SMILES: O=C1NCCCC[C@H]1Nc1nc2ccccc2c2nc(-c3cccc(Cl)c3)nn12] (3R)-3-{[2-(3-chlorophenyl)[1,2,4] triazolo[1,5-c]quinazolin-5- yl]amino}azepan-2-one LC-MS (Method 2): Rₜ = 1.46 min; MS (ESIpos): m/z = 407 [M + H]⁺ ¹H-NMR (400 MHz, DMSO-d₆): δ [ppm] = 1.28-1.39 (m, 1H), 1.52-1.66 (m, 1H), 1.81-1.95 (m, 2H), 1.98-2.08 (m, 1H), 2.26-2.35 (m, 1H), 3.12-3.23 (m, 1H), 3.44 (br d, 1H), 4.84 (br dd, 1H), 7.43-7.50 (m, 1H), 7.62-7.71 (m, 3H), 7.72-7.76 (m, 1H), 7.77-7.82 (m, 1H), 8.18-8.28 (m, 3H), 8.29-8.34 (m, 1H). |
| Example 60 | 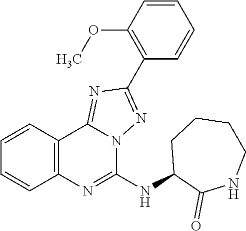 [SMILES: COc1ccccc1-c1nc2c3ccccc3nc(N[C@H]3CCCCNC3=O)n2n1] (3S)-3-{[2-(2-methoxyphenyl)[1,2,4] triazolo[1,5-c]quinazolin-5- yl]amino}azepan-2-one LC-MS (Method 2): Rₜ = 1.21 min; MS (ESIpos): m/z = 403 [M + H]⁺ ¹H-NMR (400 MHz, DMSO-d₆): δ [ppm] = 1.25-1.39 (m, 1H), 1.49-1.62 (m, 1H), 1.80-1.96 (m, 2H), 1.98-2.08 (m, 1H), 2.28-2.38 (m, 1H), 3.11-3.21 (m, 1H), 3.35-3.42 (m, 1H), 3.88 (s, 3H), 4.83 (dd, 1H), 7.13 (td, 1H), 7.24 (d, 1H), 7.45 (ddd, 1H), 7.51-7.58 (m, 1H), 7.65-7.77 (m, 3H), 7.92 (dd, 1H), 8.20 (dd, 1H), 8.28 (dd, 1H). |
| Example 61 | 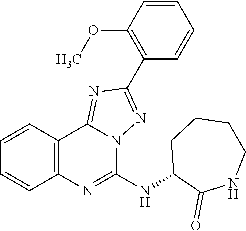 [SMILES: COc1ccccc1-c1nc2c3ccccc3nc(N[C@@H]3CCCCNC3=O)n2n1] (3R)-3-{[2-(2-methoxyphenyl)[1,2,4] triazolo[1,5-c]quinazolin-5- yl]amino}azepan-2-one LC-MS (Method 2): Rₜ = 1.21 min; MS (ESIpos): m/z = 403 [M + H]⁺ |
| Example 62 | 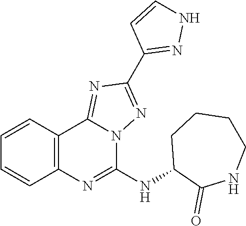 [SMILES: O=C1NCCCC[C@H]1Nc1nc2ccccc2c2nc(-c3cc[nH]n3)nn12] (3R)-3-{[2-(1H-pyrazol-3-yl)[1,2,4] triazolo[1,5-c]quinazolin-5- yl]amino}azepan-2-one LC-MS (Method 2): Rₜ = 0.97 min; MS (ESIpos): m/z = 363 [M + H]⁺ ¹H-NMR (400 MHz, DMSO-d₆): δ [ppm] = 1.25-1.40 (m, 1H), 1.48-1.62 (m, 1H), 1.80-1.96 (m, 2H), 1.98-2.07 (m, 1H), 2.33 (ddd, 1H), 3.11-3.22 (m, 1H), 3.35-3.42 (m, 1H), 4.84 (br dd, 1H), 6.95 (br s, 1H), 7.46 (t, 1H), 7.63- 7.71 (m, 2H), 7.71-7.78 (m, 1H), 7.94 (br s, 1H), 8.23 (dd, 1H), 8.28 (dd, 1H), 13.36 (br s, 1H). |
| Example 63 | 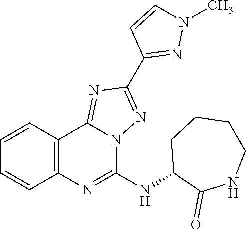 [SMILES: Cn1ccc(-c2nc3c4ccccc4nc(N[C@@H]4CCCCNC4=O)n3n2)n1] (3R)-3-{[2-(1-methyl-1H-pyrazol-3-yl) [1,2,4]triazolo[1,5-c]quinazolin-5- yl]amino}azepan-2-one LC-MS (method 2): Rt = 1.04 min; MS (ESIpos): m/z = 377 [M + H]⁺ ¹H-NMR (400 MHz, DMSO-d₆): δ [ppm] = 1.21-1.36 (m, 1H), 1.48-1.61 (m, 1H), 1.80-2.07 (m, 3H), 2.52-2.55 (m, 1H), 3.12-3.21 (m, 1H), 3.35-3.42 (m, 1H), 3.99 (s, 3H), 4.83 (dd, 1H), 6.92 (d, 1H), 7.45 (ddd, 1H), 7.64-7.77 (m, 3H), 7.89 (d, 1H), 8.21-8.30 (m, 2H). |
| Example 64 | 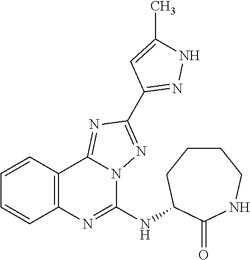 [SMILES: Cc1cc(-c2nc3c4ccccc4nc(N[C@@H]4CCCCNC4=O)n3n2)n[nH]1] (3R)-3-{[2-(5-methyl-1H-pyrazol-3-yl) [1,2,4]triazolo[1,5-c]quinazolin-5- yl]amino}azepan-2-one LC-MS (Method 2): Rₜ = 1.02 min; MS (ESIpos): m/z = 377 [M + H]⁺ ¹H-NMR (400 MHz, DMSO-d₆): δ [ppm] = 1.32 (q, 1H), 1.55 (q, 1H), 1.84-1.93 (m, 2H), 2.01-2.04 (m, 1H), 2.33 (m, 4H), 3.16 (m, 1H), 3.40 (m, 1H), 4.82 (dd, 1H), 6,70 (s, 1H), 7.45 (t, 1H), 7.67 (d, 1H), 7.73 (t, 1H), 8.22 (t, 1H), 8.28 (dd, 1H), 13.02 (br s, 1H). |
| Example 65 | 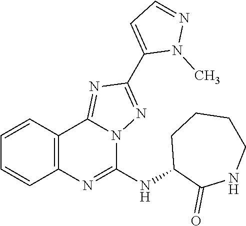 [SMILES: Cn1nccc1-c1nc2c3ccccc3nc(N[C@@H]3CCCCNC3=O)n2n1] (3R)-3-{[2-(1-methyl-1H-pyrazol-5-yl) [1,2,4]triazolo[1,5-c]quinazolin-5- yl]amino}azepan-2-one LC-MS (Method 2): Rₜ = 1.13 min; MS (ESIpos): m/z = 377 [M + H]⁺ ¹H-NMR (400 MHz, DMSO-d₆): δ [ppm] = 1.25-1.40 (m, 1H), 1.51-1.64 (m, 1H), 1.80-1.95 (m, 2H), 1.96-2.08 (m, 1H), 2.27-2.35 (m, 1H), 3.11-3.23 (m, 1H), 3.34-3.41 (m, 1H), 4.34 (s, 3H), 4.84 (dd, 1H), 7.03 (d, 1H), 7.46 (ddd, 1H), 7.62 (d, 1H), 7.65-7.71 (m, 1H), 7.73-7.81 (m, 2H), 8.20 (dd, 1H), 8.29 (dd, 1H). |
| Example 66 | 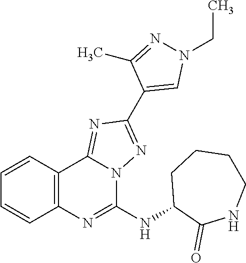 [SMILES: CCn1cc(-c2nc3c4ccccc4nc(N[C@@H]4CCCCNC4=O)n3n2)c(C)n1] (3R)-3-{[2-(1-ethyl-3-methyl-1H- pyrazol-4-yl)[1,2,4]triazolo[1,5- c]quinazolin-5-yl]amino}azepan-2-one LC-MS (Method 2): Rₜ = 1.16 min; MS (ESIpos): m/z = 405 [M + H]⁺ ¹H-NMR (400 MHz, DMSO-d₆): δ [ppm] = 0.91-0.95 (m, 1H), 1.27-1.37 (m, 1H), 1.42 (t, 3H), 1.49-1.60 (m, 1H), 1.81-1.93 (m, 2H), 1.98-2.06 (m, 1H), 2.32 (td, 1H), 2.59 (s, 3H), 3.12-3.20 (m, 1H), 4.16 (q, 2H), 4.81 (br dd, 1H), 7.43 (t, 1H), 7.63-7.67 (m, 2H), 7.70-7.75 (m, 1H), 8.17-8.25 (m, 2H), 8.41 (s, 1H). |
| Example 67 | 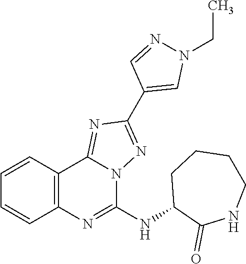 [SMILES: CCn1cc(-c2nc3c4ccccc4nc(N[C@@H]4CCCCNC4=O)n3n2)cn1] (3R)-3-{[2-(1-ethyl-1H-pyrazol-4-yl) [1,2,41triazolo[1,5-c]quinazolin-5- yl]amino}azepan-2-one LC-MS (Method 2): Rₜ = 1.09 min; MS (ESIpos): m/z = 391 [M + H]⁺ ¹H-NMR (400 MHz, DMSO-d₆): δ [ppm] = 1.26-1.37 (m, 1H), 1.45 (t, 3H), 1.48-1.59 (m, 1H), 1.81-1.93 (m, 2H), 1.98-2.05 (m, 1H), 2.28-2.34 (m, 1H), 2.51-2.53 (m, 1H), 3.12-3.20 (m, 1H), 4.25 (q, 2H), 4.82 (br dd, 1H), 7.44 (ddd, 1H), 7.60 (d, 1H), 7.66 (d, 1H), 7.70-7.75 (m, 1H), 8.08 (s, 1H), 8.19-8.26 (m, 2H), 8.53 (s, 1H). |
| Example 68 | 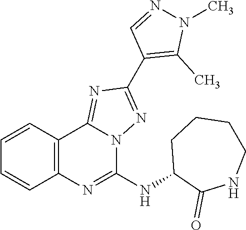 [SMILES: Cc1c(-c2nc3c4ccccc4nc(N[C@@H]4CCCCNC4=O)n3n2)cnn1C] (3R)-3-{[2-(1,5-dimethyl-1H-pyrazol-4-yl) [1,2,4]triazolo[1,5-c]quinazolin- 5-yl]amino}azepan-2-one LC-MS (Method 2): Rₜ = 1.09 min; MS (ESIpos): m/z = 391 [M + H]⁺ ¹H-NMR (400 MHz, DMSO-d₆): δ [ppm] = 1.32 (br d, 1H), 1.56 (br d, 1H), 1.85 (br d, 2H), 2.00 (br s, 1H), 2.28-2.36 (m, 2H), 2.76 (s, 3H), 3.17 (br d, 1H), 3.84 (s, 3H), 4.82 (br dd, 1H), 7.43 (t, 1H), 7.63-7.69 (m, 2H), 7.69-7.75 (m, 1H), 7.99 (s, 1H), 8.18 (br t, 1H), 8.26 (d, 1H). |
| Example 69 | 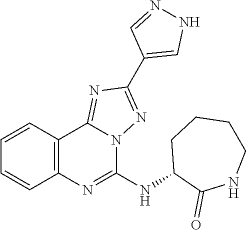 [SMILES: O=C1NCCCC[C@H]1Nc1nc2ccccc2c2nc(-c3cn[nH]c3)nn12] (3R)-3-{[2-(1H-pyrazol-4-yl)[1,2,4] triazolo[1,5-c]quinazolin-5- yl]amino}azepan-2-one LC-MS (Method 2): Rₜ = 0.95 min; MS (ESIpos): m/z = 363 [M + H]⁺ ¹H-NMR (400 MHz, DMSO-d₆): δ [ppm] = 1.25-1.41 (m, 1H), 1.47-1.63 (m, 1H), 1.80-1.96 (m, 2H), 1.98-2.09 (m, 1H), 2.26-2.37 (m, 1H), 3.10-3.23 (m, 1H), 4.83 (dd, 1H), 7.44 (ddd, 1H), 7.61 (d, 1H), 7.64-7.68 (m, 1H), 7.69- 7.76 (m, 1H), 8.11-8.50 (m, 1H), 8.21 (br dd, 1H), 8.25 (dd, 1H), 8.28- 8.42 (m, 1H), 13.34 (br s, 1H). |
| Example 70 | 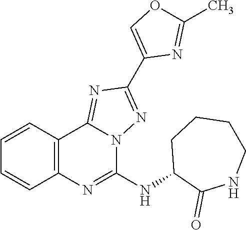 [SMILES: Cc1nc(-c2nc3c4ccccc4nc(N[C@@H]4CCCCNC4=O)n3n2)co1] (3R)-3-{[2-(2-methyl-1,3-oxazol-4-yl) [1,2,4]triazolo[1,5-c]quinazolin-5- yl]amino}azepan-2-one LC-MS (Method 2): Rt = 1.06 min; MS (ESIpos): m/z = 378 [M + H]⁺ ¹H-NMR (400 MHz, DMSO-d₆): δ [ppm] = 1.21-1.39 (m, 1H), 1.47-1.60 (m, 1H), 1.80-1.95 (m, 2H), 1.98-2.10 (m, 1H), 2.29-2.37 (m, 1H), 2.55 (s, 3H), 3.11-3.21 (m, 1H), 4.82 (br dd, 1H), 7.41-7.49 (m, 1H), 7.64-7.78 (m, 3H), 8.20-8.27 (m, 2H), 8.77-8.82 (m, 1H). |

The following examples were prepared analogously to example 1 starting from intermediate 48:

| | Structure |
|---|---|
| | IUPAC-Name |
| | LC-MS (method): Retention time; Mass found |
| Example | ¹H-NMR |
| Example 71 | 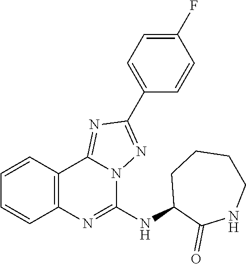 [SMILES: O=C1NCCCC[C@@H]1Nc1nc2ccccc2c2nc(-c3ccc(F)cc3)nn12] |
| | (3S)-3-{[2-(4-fluorophenyl)[1,2,4]triazolo[1,5-c]quinazolin-5- |
| | yl]amino}azepan-2-one |
| | LC-MS (Method 2): Rₜ = 1.36 min; MS (ESIpos): m/z = 391 [M + H]⁺ |
| | ¹H-NMR (400 MHz, DMSO-d₆): δ [ppm] = 1.26-1.39 (m, 1H), 1.50-1.63 (m, |
| | 1H), 1.81-1.95 (m, 2H), 1.98-2.07 (m, 1H), 2.28-2.36 (m, 1H), 3.12 (s, |
| | 1H), 4.84 (dd, 1H), 7.40-7.49 (m, 3H), 7.65-7.70 (m, 1H), 7.72-7.78 (m, |
| | 2H), 8.22 (dd, 1H), 8.28-8.37 (m, 3H). |
| Example 72 | 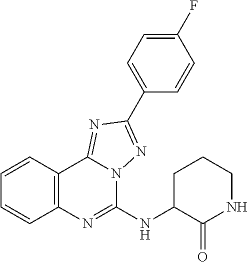 [SMILES: O=C1NCCCC1Nc1nc2ccccc2c2nc(-c3ccc(F)cc3)nn12] |
| | 3-{[2-(4-fluorophenyl)[1,2,4]triazolo[1,5-c]quinazolin-5- |
| | yl]amino}piperidin-2-one |
| | LC-MS (Method 2): Rₜ = 0.89 min; MS (ESIpos): m/z = 377 [M + H]⁺ |
| | ¹H-NMR (400 MHz, DMSO-d₆): δ [ppm] = 1.87-1.97 (m, 2H), 2.09 (qd, 1H), |
| | 2.25-2.35 (m, 1H), 3.21-3.29 (m, 2H), 4.70 (dt, 1H), 7.40-7.49 (m, 3H), |
| | 7.61-7.66 (m, 1H), 7.69-7.76 (m, 1H), 7.81 (br s, 1H), 8.18 (d, 1H), 8.29 |
| | (dd, 1H), 8.31-8.37 (m, 2H). |
| Example 73 | 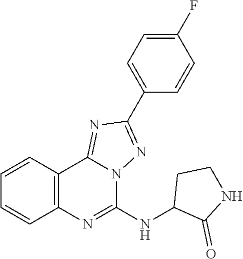 [SMILES: O=C1NCCC1Nc1nc2ccccc2c2nc(-c3ccc(F)cc3)nn12] |
| | 3-{[2-(4-fluorophenyl)[1,2,4]triazolo[1,5-c]quinazolin-5- |
| | yl]amino}pyrrolidin-2-one |
| | LC-MS (Method 2): Rₜ = 1.14 min; MS (ESIpos): m/z = 363 [M + H]⁺ |
| | ¹H-NMR (400 MHz, DMSO-d₆): δ [ppm] = 2.25-2.38 (m, 1H), 4.94 (dt, 1H), |
| | 7.40-7.49 (m, 3H), 7.63 (d, 1H), 7.70-7.76 (m, 1H), 8.01 (s, 1H), 8.27- |
| | 8.38 (m, 4H). |
| Example 74 | 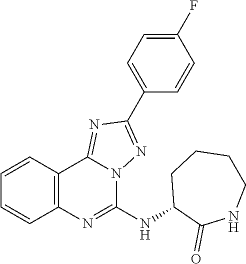 [SMILES: O=C1NCCCC[C@H]1Nc1nc2ccccc2c2nc(-c3ccc(F)cc3)nn12] |
| | (3R)-3-{[2-(4-fluorophenyl)[1,2,4]triazolo[1,5-c]quinazolin-5- |
| | yl]amino}azepan-2-one |
| | LC-MS (Method 2): Rₜ = 0.90 min; MS (ESIpos): m/z = 391 [M + H]⁺ |
| | ¹H-NMR (400 MHz, DMSO-d₆): δ [ppm] = 1.26-1.39 (m, 1H), 1.50-1.63 (m, |
| | 1H), 1.81-1.95 (m, 2H), 1.98-2.07 (m, 1H), 2.32 (td, 1H), 3.12-3.22 (m, |
| | 1H), 3.36-3.42 (m, 1H), 4.84 (br dd, 1H), 7.39-7.50 (m, 3H), 7.65-7.70 |
| | (m, 1H), 7.72-7.78 (m, 2H), 8.22 (dd, 1H), 8.28-8.37 (m, 3H). |

The following examples were prepared analogously to example 1 starting from intermediate 69:

| | Structure |
|---|---|
| | IUPAC-Name |
| | LC-MS (method): Retention time; Mass found |
| Example | ¹H-NMR |
| Example 75 | 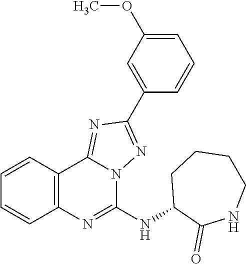 [SMILES: COc1cccc(-c2nc3c4ccccc4nc(N[C@@H]4CCCCNC4=O)n3n2)c1] |
| | (3R)-3-{[2-(3-methoxyphenyl)[1,2,4]triazolo[1,5-c]quinazolin-5- |
| | yl]amino}azepan-2-one |
| | LC-MS (Method 2): Rₜ = 1.31 min; MS (ESIpos): m/z = 403 [M + H]⁺ |
| | ¹H-NMR (400 MHz, DMSO-d₆): δ [ppm] = 1.33 (q, 1H), 1.52-1.65 (m, 1H), |
| | 1.80-2.08 (m, 3H), 2.31 (brd, 1H), 3.11-3.23 (m, 1H), 3.36 (brd, 1H), 3.89 |
| | (s, 3H), 4.84 (dd, 1H), 7.15 (ddd, 1H), 7.42-7.49 (m, 1H), 7.52 (t, 1H), 7.64- |
| | 7.70 (m, 1H), 7.71-7.77 (m, 2H), 7.79 (dd, 1H), 7.88 (dt, 1H), 8.21 (dd, 1H), |
| | 8.31 (dd, 1H). |
| Example 76 | 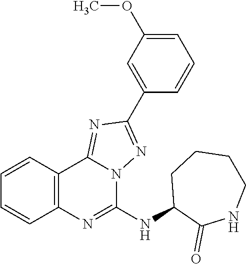 [SMILES: COc1cccc(-c2nc3c4ccccc4nc(N[C@H]4CCCCNC4=O)n3n2)c1] |
| | (3S)-3-{[2-(3-methoxyphenyl)[1,2,4]triazolo[1,5-c]quinazolin-5- |
| | yl]amino}azepan-2-one |
| | LC-MS (Method 2): Rₜ = 1.31 min; MS (ESIpos): m/z = 403 [M + H]⁺ |
| Example 77 | 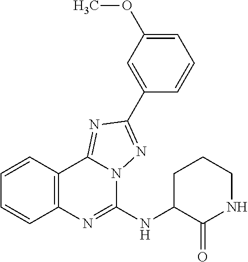 [SMILES: COc1cccc(-c2nc3c4ccccc4nc(NC4CCCNC4=O)n3n2)c1] |
| | 3-{[2-(3-methoxyphenyl)[1,2,4]triazolo[1,5-c]quinazolin-5- |
| | yl]amino}piperidin-2-one |
| | LC-MS (Method 2): Rₜ = 1.15 min; MS (ESIpos): m/z = 389 [M + H]⁺ |
| | ¹H-NMR (400 MHz, DMSO-d₆): δ [ppm] = 1.86-1.98 (m, 2H), 2.05-2.17 (m, |
| | 1H), 2.24-2.32 (m, 1H), 3.22-3.29 (m, 2H), 3.89 (s, 3H), 4.72 (dt, 1H), 7.14 |
| | (ddd, 1H), 7.44 (ddd, 1H), 7.52 (t, 1H), 7.61-7.66 (m, 1H), 7.70-7.76 (m, |
| | 1H), 7.81 (br s, 1H), 7.83 (dd, 1H), 7.89-7.92 (m, 1H), 8.21 (d, 1H), 8.30 |
| | (dd, 1H). |
| Example 78 | 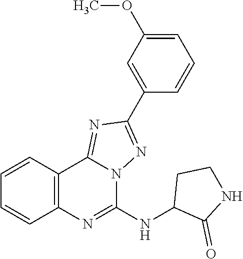 [SMILES: COc1cccc(-c2nc3c4ccccc4nc(NC4CCNC4=O)n3n2)c1] |
| | 3-{[2-(3-methoxyphenyl)[1,2,4]triazolo[1,5-c]quinazolin-5- |
| | yl]amino}piperidin-2-one |
| | LC-MS (Method 2): Rₜ = 1.09 min; MS (ESIpos): m/z = 375 [M + H]⁺ |
| | ¹H-NMR (400 MHz, DMSO-d₆): δ [ppm] = 2.25-2.40 (m, 1H), 3.32-3.37 (m, |
| | 2H), 3.89 (s, 3H), 4.90-5.01 (m, 1H), 7.14 (ddd, 1H), 7.41-7.47 (m, 1H), |
| | 7.52 (t, 1H), 7.63 (d, 1H), 7.71-7.77 (t, 1H), 7.84 (dd, 1H), 7.91 (dt, 1H) |
| | 8.01 (s, 1H), 8.28-8.36 (m, 2H). |
| Example 79 | 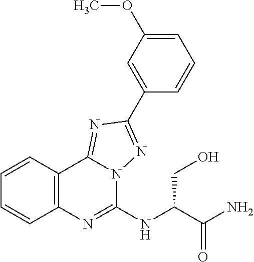 [SMILES: COc1cccc(-c2nc3c4ccccc4nc(N[C@H](CO)C(N)=O)n3n2)c1] |
| | N²-[2-(3-methoxyphenyl)[1,2,4]triazolo[1,5-c]quinazolin-5-yl]-D- |
| | serinamide |
| | LC-MS (Method 2): Rₜ = 0.98 min; MS (ESIneg): m/z = 377 [M − H]⁻ |
| | ¹H-NMR (400 MHz, DMSO-d₆): δ [ppm] = 3.90 (s, 3H), 3.96 (t, 2H), 4.69-4.76 |
| | (m, 1H), 5.16-5.21 (m, 1H), 7.15 (ddd, 1H), 7.32 (s, 1H), 7.43-7.49 (m, |
| | 1H), 7.49-7.58 (m, 2H), 7.62-7.70 (m, 2H), 7.71-7.77 (m, 1H), 7.83 (dd, |
| | 1H), 7.92 (dt, 1H), 8.32 (dd, 1H). |
| Example 80 | 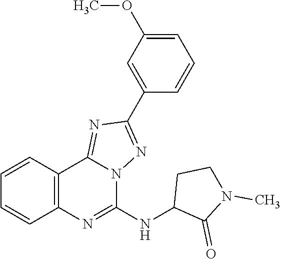 [SMILES: COc1cccc(-c2nc3c4ccccc4nc(NC4CCN(C)C4=O)n3n2)c1] |
| | 3-{[2-(3-methoxyphenyl)[1,2,4]triazolo[1,5-c]quinazolin-5-yl]amino}-1- |
| | methylpyrrolidin-2-one |
| | LC-MS (Method 2): Rₜ = 1.15 min; MS (ESIpos): m/z = 389 [M + H]⁺ |
| | ¹H-NMR (400 MHz, DMSO-d₆): δ [ppm] = 2.83 (s, 3H), 3.44 (br dd, 4H), 3.89 |
| | (s, 3H), 4.93-5.05 (m, 1H), 7.14 (br dd, 1H), 7.44 (br t, 1H), 7.52 (br t, 1H), |
| | 7.61 (br d, 1H), 7.70-7.77 (m, 1H), 7.83 (br s, 1H), 7.90 (br d, 1H), 8.31 (br |
| | d, 1H), 8.46 (br d, 1H). |
| Example 81 | 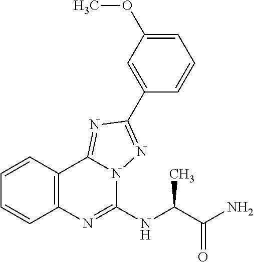 [SMILES: COc1cccc(-c2nc3c4ccccc4nc(N[C@@H](C)C(N)=O)n3n2)c1] |
| | N²-[2-(3-methoxyphenyl[1,2,4]triazolo[1,5-c]quinazolin-5-yl]-L- |
| | alaninamide |
| | LC-MS (Method 2): Rₜ = 1.08 min; MS (ESIpos): m/z = 363 [M + H]⁺ |
| | ¹H-NMR (400 MHz, DMSO-d₆): δ [ppm] = 1.55 (d, 3H), 3.89 (s, 3H), 4.76 |
| | (quin, 1H), 7.14 (ddd, 1H), 7.26 (s, 1H), 7.45 (ddd, 1H), 7.51 (t, 1H), 7.61- |
| | 7.65 (m, 1H), 7.67 (s, 1H), 7.70-7.76 (m, 1H), 7.80 (d, 1H), 7.83 (dd, 1H), |
| | 7.91 (dt, 1H), 8.31 (dd, 1H). |
| Example 82 | 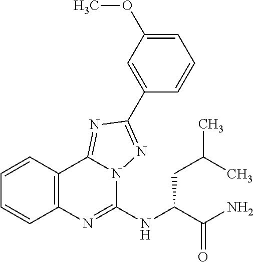 [SMILES: COc1cccc(-c2nc3c4ccccc4nc(N[C@H](CC(C)C)C(N)=O)n3n2)c1] |
| | N²-[2-(3-methoxyphenyl)[1,2,4]triazolo[1,5-c]quinazolin-5-yl]-D- |
| | leucinamide |
| | LC-MS (Method 2): Rₜ = 1.27 min; MS (ESIneg): m/z = [M − H]⁻ |
| | ¹H-NMR (400 MHz, DMSO-d₆): δ [ppm] = 0.95 (dd, 6H), 1.68-1.82 (m, 2H), |
| | 1.88-1.98 (m, 1H), 3.89 (s, 3H), 4.77-4.84 (m, 1H), 7.14 (ddd, 1H), 7.19 (s, |
| | 1H), 7.44 (ddd, 1H), 7.51 (t, 1H), 7.59-7.64 (m, 1H), 7.65 (s, 1H), 7.70- |
| | 7.75 (m, 1H), 7.81 (d, 1H), 7.85 (dd, 1H), 7.93 (dt, 1H), 8.30 (dd, 1H). |
| Example 83 | 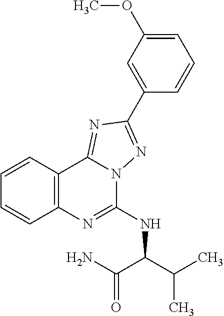 [SMILES: COc1cccc(-c2nc3c4ccccc4nc(N[C@H](C(N)=O)C(C)C)n3n2)c1] |
| | N²-[2-(3-methoxyphenyl)[1,2,4]triazolo[1,5-c]quinazolin-5-yl]-L- |
| | valinamide |
| | LC-MS (Method 2): Rₜ = 1.21 min; MS (ESIpos): m/z = 391 [M + H]⁺ |
| | ¹H-NMR (400 MHz, DMSO-d₆): δ [ppm] = 1.03 (dd, 6H), 2.27-2.37 (m, 1H), |
| | 3.89 (s, 3H), 4.70 (dd, 1H), 7.15 (ddd, 1H), 7.28 (d, 1H), 7.37 (s, 1H), 7.43- |
| | 7.48 (m, 1H), 7.52 (t, 1H), 7.64 (d, 1H), 7.71-7.77 (m, 1H), 7.79 (s, 1H), |
| | 7.82 (dd, 1H), 7.92 (dt, 1H), 8.31 (dd, 1H). |
| Example 84 | 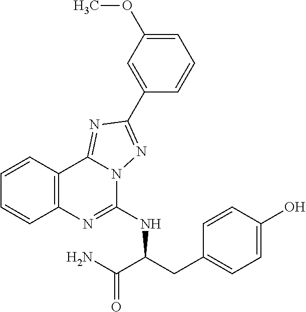 [SMILES: COc1cccc(-c2nc3c4ccccc4nc(N[C@@H](Cc4ccc(O)cc4)C(N)=O)n3n2)c1] |
| | N²-[2-(3-methoxyphenyl)[1,2,4]triazolo[1,5-c]quinazolin-5-yl]-L- |
| | tyrosinamide |
| | LC-MS (Method 2): Rₜ = 1.09 min; MS (ESIpos): m/z = 455 [M + H]⁺ |
| | ¹H-NMR (400 MHz, DMSO-d₆): δ [ppm] = 3.14-3.27 (m, 2H), 3.89 (s, 3H), |
| | 4.88 (td, 1H), 6.61 (d, 2H), 7.09-7.17 (m, 3H), 7.31 (s, 1H), 7.41-7.47 (m, |
| | 1H), 7.52 (t, 1H), 7.58-7.66 (m, 2H), 7.68-7.78 (m, 2H), 7.81 (dd, 1H), 7.89 |
| | (dt, 1H), 8.29 (dd, 1H), 9.15 (s, 1H). |
| Example 85 | 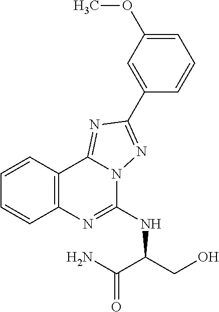 [SMILES: COc1cccc(-c2nc3c4ccccc4nc(N[C@@H](CO)C(N)=O)n3n2)c1] |
| | N²-[2-(3-methoxyphenyl)[1,2,4]triazolo[1,5-c]quinazolin-5-yl]-L- |
| | serinamide |
| | LC-MS (Method 2): Rₜ = 0.97 min; MS (ESIpos): m/z = 379 [M + H]⁺ |
| | ¹H-NMR (400 MHz, DMSO-d₆): δ [ppm] = 3.90 (s, 3H), 3.96 (t, 2H), 4.69-4.76 |
| | (m, 1H), 5.16-5.22 (m, 1H), 7.15 (ddd, 1H), 7.32 (s, 1H), 7.43-7.49 (m, |
| | 1H), 7.49-7.58 (m, 2H), 7.64 (d, 1H), 7.68 (s, 1H), 7.71-7.77 (m, 1H), 7.83 |
| | (dd, 1H), 7.92 (dt, 1H), 8.32 (dd, 1H). |
| Example 86 | 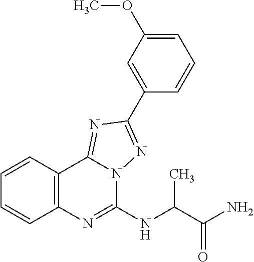 [SMILES: COc1cccc(-c2nc3c4ccccc4nc(NC(C)C(N)=O)n3n2)c1] |
| | N²-[2-(3-methoxyphenyl)[1,2,4]triazolo[1,5-c]quinazolin-5-yl]alaninamide |
| | LC-MS (Method 2): Rₜ = 1.10 min; MS (ESIpos): m/z = 363 [M + H]⁺ |
| | ¹H-NMR (400 MHz, DMSO-d₆): δ [ppm] = 1.55 (d, 3H), 3.89 (s, 3H), 4.76 |
| | (quin, 1H), 7.14 (ddd, 1H), 7.26 (s, 1H), 7.45 (ddd, 1H), 7.51 (t, 1H), 7.61- |
| | 7.65 (m, 1H), 7.67 (s, 1H), 7.69-7.77 (m, 1H), 7.79 (d, 1H), 7.83 (dd, 1H), |
| | 7.91 (dt, 1H), 8.31 (dd, 1H). |
| Example 87 | 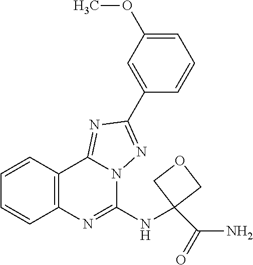 [SMILES: COc1cccc(-c2nc3c4ccccc4nc(NC4(C(N)=O)COC4)n3n2)c1] |
| | 3-{[2-(3-methoxyphenyl)[1,2,4]triazolo[1,5-c]quinazolin-5- |
| | yl]amino}oxetane-3-carboxamide |
| | LC-MS (Method 2): Rₜ = 1.02 min; MS (ESIpos): m/z = 391 [M + H]⁺ |
| | ¹H-NMR (400 MHz, DMSO-d₆): δ [ppm] = 3.90 (s, 3H), 4.89 (d, 2H), 4.98 (d, |
| | 2H), 7.15 (ddd, 1H), 7.17 (br s, 1H), 7.42-7.48 (m, 2H), 7.49-7.56 (m, 2H), |
| | 7.67-7.73 (m, 1H), 7.87 (dd, 1H), 7.94 (dt, 1H), 8.31 (dd, 1H), 9.04-9.06 |
| | (m, 1H). |
| Example 88 | 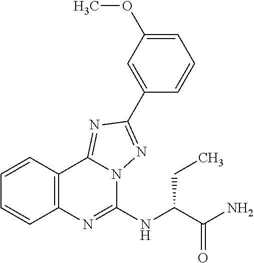 [SMILES: CC[C@@H](Nc1nc2ccccc2c2nc(-c3cccc(OC)c3)nn12)C(N)=O] |
| | (2R)-2-{[2-(3-methoxyphenyl)[1,2,4]triazolo[1,5-c]quinazolin-5- |
| | yl]amino}butanamide |
| | LC-MS (Method 2): Rₜ = 1.17 min; MS (ESIpos): m/z = 377 [M + H]⁺ |
| | ¹H-NMR (400 MHz, DMSO-d₆): δ [ppm] = 0.97 (t, 3H), 1.88-2.00 (m, 1H), |
| | 2.01-2.13 (m, 1H), 3.89 (s, 3H), 4.69 (td, 1H), 7.14 (ddd, 1H), 7.30 (s, 1H), |
| | 7.45 (td, 1H), 7.52 (t, 1H), 7.63 (d, 2H), 7.69-7.76 (m, 2H), 7.83 (dd, 1H), |
| | 7.92 (dt, 1H), 8.31 (dd, 1H). |
| Example 89 | 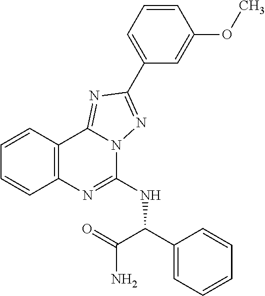 [SMILES: COc1cccc(-c2nc3c4ccccc4nc(N[C@@H](C(N)=O)c4ccccc4)n3n2)c1] |
| | (2R)-2-{[2-(3-methoxyphenyl)[1,2,4]triazolo[1,5-c]quinazolin-5-yl]amino}- |
| | 2-phenylacetamide |
| | LC-MS (Method 2): Rₜ = 1.26 min; MS (ESIneg): m/z = 423 [M − H]⁻ |
| | ¹H-NMR (400 MHz, DMSO-d₆): δ [ppm] = 3.89 (s, 3H), 5.87 (d, 1H), 7.15 (ddd, |
| | 1H), 7.28-7.34 (m, 1H), 7.36-7.42 (m, 2H), 7.46 (ddd, 1H), 7.52 (t, 1H), |
| | 7.56 (s, 1H), 7.60 (d, 1H), 7.62-7.67 (m, 2H), 7.69-7.75 (m, 1H), 7.79 (d, |
| | 1H), 7.82 (dd, 1H), 7.91 (dt, 1H), 8.01 (s, 1H), 8.31 (dd, 1H). |
| Example 90 | 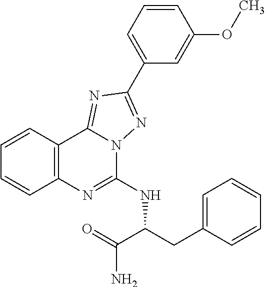 [SMILES: COc1cccc(-c2nc3c4ccccc4nc(N[C@H](Cc4ccccc4)C(N)=O)n3n2)c1] |
| | N²-[2-(3-methoxyphenyl)[1,2,4]triazolo[1,5-c]quinazolin-5-yl]-D- |
| | phenylalaninamide |
| | LC-MS (Method 2): Rₜ = 1.28 min; MS (ESIpos): m/z = 439 [M + H]⁺ |
| | ¹H-NMR (400 MHz, DMSO-d₆) δ [ppm] = 3.89 (s, 3H), 4.97 (td, 1H), 7.10- |
| | 7.17 (m, 2H), 7.20-7.26 (m, 2H), 7.32-7.37 (m, 3H), 7.44 (ddd, 1H), 7.52 (t, |
| | 1H), 7.59-7.63 (m, 1H), 7.72 (ddd, 1H), 7.77 (d, 1H), 7.80 (s, 1H), 7.81 (dd, |
| | 1H), 7.89 (dt, 1H), 8.28 (dd, 1H). |
| Example 91 | 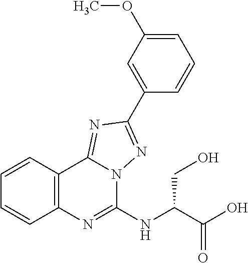 [SMILES: COc1cccc(-c2nc3c4ccccc4nc(N[C@H](CO)C(=O)O)n3n2)c1] |
| | N-[2-(3-methoxyphenyl)[1,2,4]triazolo[1,5-c]quinazolin-5-yl]-D-serine |
| | LC-MS (Method 2): Rₜ = 0.65 min; MS (ESIpos): m/z = 380 [M + H]⁺ |
| | ¹H-NMR (400 MHz, DMSO-d₆): δ [ppm] = 3.83-3.89 (m, 1H), 3.90 (s, 3H), |
| | 4.03 (dd, 1H), 4.57 (br d, 1H), 7.15 (ddd, 1H), 7.45 (ddd, 1H), 7.52 (t, 1H), |
| | 7.63-7.68 (m, 1H), 7.70-7.76 (m, 2H), 7.82 (dd, 1H), 7.91 (dt, 1H), 8.31 |
| | (dd, 1H). |
| Example 92 | 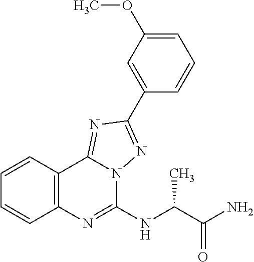 [SMILES: COc1cccc(-c2nc3c4ccccc4nc(N[C@H](C)C(N)=O)n3n2)c1] |
| | N²-[2-(3-methoxyphenyl)[1,2,4]triazolo[1,5-c]quinazolin-5-yl]-D- |
| | alaninamide |
| | LC-MS (Method 2): Rₜ = 1.10 min; MS (ESIpos): m/z = 363 [M + H]⁺ |
| Example 93 | 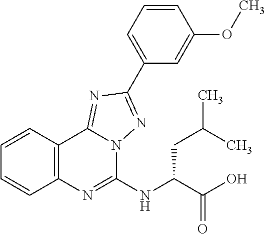 [SMILES: COc1cccc(-c2nc3c4ccccc4nc(N[C@H](CC(C)C)C(=O)O)n3n2)c1] |
| | N-[2-(3-methoxyphenyl)[1,2,4]triazolo[1,5-c]quinazolin-5-yl]-D-leucine |
| | LC-MS (Method 2): Rₜ = 0.80 min; MS (ESIpos): m/z = 406 [M + H]⁺ |
| | ¹H-NMR (400 MHz, DMSO-d₆): δ [ppm] = 0.96 (dd, 6H), 1.69-1.89 (m, 2H), |
| | 2.02-2.13 (m, 1H), 3.90 (s, 3H), 4.76-4.85 (m, 1H), 7.15 (ddd, 1H), 7.44 |
| | (ddd, 1H), 7.52 (t, 1H), 7.62 (d, 1H), 7.68-7.75 (m, 1H), 7.86 (dd, 1H), 7.93 |
| | (dt, 1H), 8.19 (d, 1H), 8.31 (dd, 1H). |
| Example 94 | 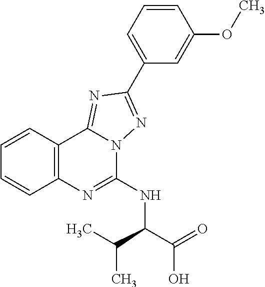 [SMILES: COc1cccc(-c2nc3c4ccccc4nc(N[C@@H](C(=O)O)C(C)C)n3n2)c1] |
| | N-[2-(3-methoxyphenyl)[1,2,4]triazolo[1,5-c]quinazolin-5-yl]-D-valine |
| | LC-MS (Method 2): Rₜ = 0.76 min; MS (ESIpos): m/z = 392 [M + H]⁺ |
| | ¹H-NMR (400 MHz, DMSO-d₆): δ [ppm] = 1.06 (dd, 6H), 2.38-2.46 (m, 1H), |
| | 3.90 (s, 3H), 4.54-4.64 (m, 1H), 7.12-7.17 (m, 1H), 7.46 (t, 1H), 7.52 (t, |
| | 1H), 7.58 (br d, 1H), 7.62-7.67 (m, 1H), 7.70-7.76 (m, 1H), 7.83-7.86 (m, |
| | 1H), 7.93 (d, 1H), 8.31 (dd, 1H). |
| Example 95 | 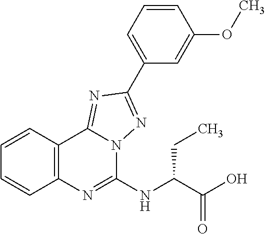 [SMILES: CC[C@@H](Nc1nc2ccccc2c2nc(-c3cccc(OC)c3)nn12)C(=O)O] |
| | (2R)-2-{[2-(3-methoxyphenyl)[1,2,4]triazolo[1,5-c]quinazolin-5- |
| | yl]amino}butanoic acid |
| | LC-MS (Method 2): Rₜ = 0.72 min; MS (ESIpos): m/z = 378 [M + H]⁺ |
| | ¹H-NMR (400 MHz, DMSO-d₆): δ [ppm] = 1.02 (br t, 3H), 2.02-2.13 (m, 2H), |
| | 3.90 (s, 3H), 4.24 (dd, 1H), 7.15 (dd, 1H), 7.42-7.48 (m, 1H), 7.52 (t, 1H), |
| | 7.63 (d, 1H), 7.70-7.76 (m, 1H), 7.85 (d, 1H), 7.93 (d, 1H), 8.04 (d, 1H), |
| | 8.09 (s, 1H), 8.31 (dd, 1H). |
| Example 96 | 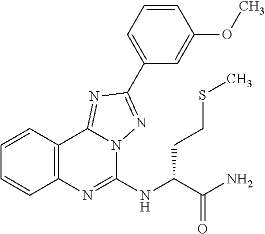 [SMILES: COc1cccc(-c2nc3c4ccccc4nc(N[C@H](CCSC)C(N)=O)n3n2)c1] |
| | N²-[2-(3-methoxyphenyl)[1,2,4]triazolo[1,5-c]quinazolin-5-yl]-D- |
| | methioninamide |
| | LC-MS (Method 2): Rₜ = 1.21 min; MS (ESIpos): m/z = 423 [M + H]⁺ |
| | ¹H-NMR (400 MHz, DMSO-d₆): δ [ppm] = 2.06 (s, 3H), 2.17-2.32 (m, 2H), |
| | 2.53-2.66 (m, 2H), 3.89 (s, 3H), 4.84 (td, 1H), 7.14 (ddd, 1H), 7.29 (s, 1H), |
| | 7.45 (td, 1H), 7.52 (t, 1H), 7.63 (d, 1H), 7.68 (s, 1H), 7.70-7.76 (m, 1H), |
| | 7.84 (dd, 1H), 7.92 (d, 2H), 8.31 (dd, 1H). |
| Example 97 | 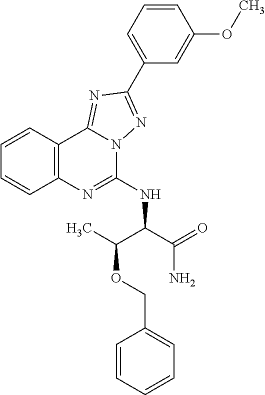 [SMILES: COc1cccc(-c2nc3c4ccccc4nc(N[C@@H](C(N)=O)[C@H](C)OCc4ccccc4)n3n2)c1] |
| | O-benzyl-N²-[2-(3-methoxyphenyl)[1,2,4]triazolo[1,5-c]quinazolin-5-yl]- |
| | D-threoninamide |
| | LC-MS (Method 2): Rₜ = 1.39 min; MS (ESIneg): m/z = 481 [M − H]⁻ |
| | ¹H-NMR (400 MHz, DMSO-d₆): δ [ppm] = 1.29 (d, 3H), 3.88 (s, 3H), 4.30 (qd, |
| | 1H), 4.57-4.63 (m, 1H), 4.68-4.73 (m, 1H), 4.78 (dd, 1H), 7.11-7.18 (m, |
| | 2H), 7.26-7.37 (m, 3H), 7.37-7.42 (m, 2H), 7.43-7.55 (m, 3H), 7.63-7.68 |
| | (m, 1H), 7.72-7.78 (m, 2H), 7.79 (dd, 1H), 7.89 (dt, 1H), 8.33 (dd, 1H). |
| Example 98 | 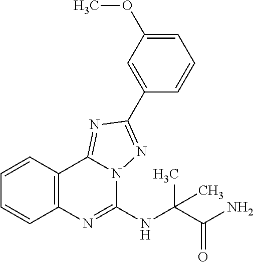 [SMILES: COc1cccc(-c2nc3c4ccccc4nc(NC(C)(C)C(N)=O)n3n2)c1] |
| | N²-[2-(3-methoxyphenyl)[1,2,4]triazolo[1,5-c]quinazolin-5-yl]-2- |
| | methylalaninamide |
| | LC-MS (Method 2): Rₜ = 1.18 min; MS (ESIpos): m/z = 377 [M + H]⁺ |
| | ¹H-NMR (400 MHz, DMSO-d₆): δ [ppm] = 1.79 (s, 6H), 3.89 (s, 3H), 7.14 (ddd, |
| | 1H), 7.31 (s, 1H), 7.45 (td, 1H), 7.52 (t, 1H), 7.60-7.67 (m, 2H), 7.70-7.76 |
| | (m, 1H), 7.81 (dd, 1H), 7.87-7.91 (m, 2H), 8.30 (dd, 1H). |
| Example 99 | 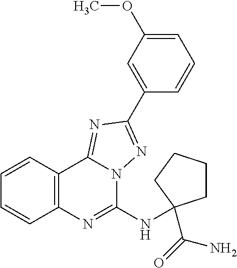 [SMILES: COc1cccc(-c2nc3c4ccccc4nc(NC4(C(N)=O)CCCC4)n3n2)c1] |
| | 1-{[2-(3-methoxyphenyl)[1,2,4]triazolo[1,5-c]quinazolin-5- |
| | yl]amino}cyclopentane-1-carboxamide |
| | LC-MS (Method 2): Rₜ = 1.22 min; MS (ESIpos): m/z = 403 [M + H]⁺ |
| | ¹H-NMR (400 MHz, DMSO-d₆): δ [ppm] = 1.69-1.83 (m, 4H), 2.29-2.38 (m, |
| | 4H), 3.89 (s, 3H), 6.88 (s, 1H), 7.14 (ddd, 1H), 7.34 (s, 1H), 7.41-7.48 (m, |
| | 1H), 7.51 (t, 1H), 7.55-7.61 (m, 2H), 7.67-7.74 (m, 1H), 7.86 (dd, 1H), 7.94 |
| | (dd, 1H), 8.30 (dd, 1H). |
| Example 100 | 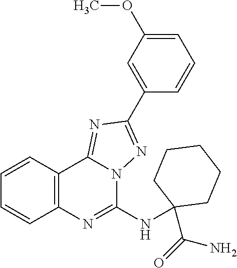 [SMILES: COc1cccc(-c2nc3c4ccccc4nc(NC4(C(N)=O)CCCCC4)n3n2)c1] |
| | 1-{[2-(3-methoxyphenyl)[1,2,4]triazolo[1,5-c]quinazolin-5- |
| | yl]amino}cyclohexane-1-carboxamide |
| | LC-MS (Method 2): Rₜ = 1.28 min; MS (ESIpos): m/z = 417 [M + H]⁺ |
| | ¹H-NMR (400 MHz, DMSO-d₆): δ [ppm] = 1.25-1.39 (m, 1H), 1.43-1.57 (m, |
| | 2H), 1.62 (br d, 3H), 1.86-2.00 (m, 2H), 2.54 (br s, 1H), 3.89 (s, 3H), 6.90 |
| | (s, 1H), 6.95 (s, 1H), 7.15 (ddd, 1H), 7.38 (s, 1H), 7.43-7.49 (m, 1H), 7.52 (t, |
| | 1H), 7.57 (d, 1H), 7.68-7.76 (m, 1H), 7.86 (dd, 1H), 7.94 (dt, 1H), 8.31 (dd, |
| | 1H). |
| Example 101 | 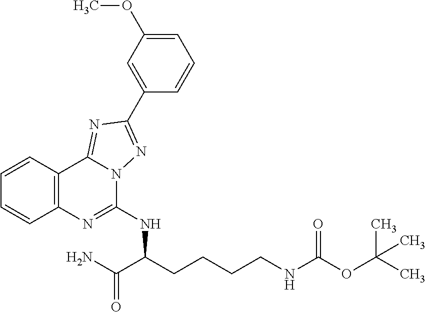 [SMILES: COc1cccc(-c2nc3c4ccccc4nc(N[C@@H](CCCCNC(=O)OC(C)(C)C)C(N)=O)n3n2)c1] |
| | tert-butyl[(5S)-6-amino-5-{[2-(3-methoxyphenyl)[1,2,4]triazolo[1,5- |
| | c]quinazolin-5-yl]amino}-6-oxohexyl]carbamate |
| | LC-MS (Method 2): Rₜ = 1.28 min; MS (ESIpos): m/z = 520 [M + H]⁺ |
| | ¹H-NMR (400 MHz, DMSO-d₆): δ [ppm] = 1.29 (s, 9H), 1.34-1.47 (m, 4H), |
| | 1.87-2.04 (m, 2H), 2.89 (q, 2H), 3.89 (s, 3H), 4.73 (td, 1H), 6.75 (t, 1H), |
| | 7.14 (ddd, 1H), 7.26 (s, 1H), 7.44 (ddd, 1H), 7.51 (t, 1H), 7.60-7.65 (m, 1H), |
| | 7.66-7.77 (m, 3H), 7.83 (dd, 1H), 7.92 (dt, 1H), 8.30 (dd, 1H). |
| Example 102 | 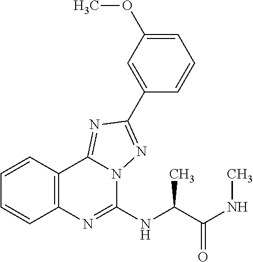 [SMILES: CNC(=O)[C@H](C)Nc1nc2ccccc2c2nc(-c3cccc(OC)c3)nn12] |
| | N²-[2-(3-methoxyphenyl)[1,2,4]triazolo[1,5-c]quinazolin-5-yl]-N-methyl-L- |
| | alaninamide |
| | LC-MS (Method 2): Rₜ = 1.13 min; MS (ESIpos): m/z = 377 [M + H]⁺ |
| | ¹H-NMR (400 MHz, DMSO-d₆): δ [ppm] = 1.53 (d, 3H), 2.63 (d, 3H), 3.89 (s, |
| | 3H), 4.80 (quin, 1H), 7.14 (ddd, 1H), 7.45 (ddd, 1H), 7.52 (t, 1H), 7.63 (d, |
| | 1H), 7.70-7.76 (m, 1H), 7.84 (dd, 1H), 7.89-7.94 (m, 2H), 8.09 (q, 1H), |
| | 8.31 (dd, 1H). |
| Example 103 | 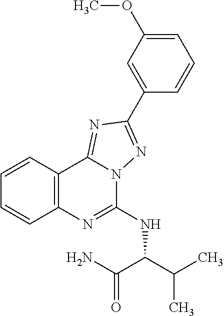 [SMILES: COc1cccc(-c2nc3c4ccccc4nc(N[C@@H](C(N)=O)C(C)C)n3n2)c1] |
| | N²-[2-(3-methoxyphenyl)[1,2,4]triazolo[1,5-c]quinazolin-5-yl]-D- |
| | valinamide |
| | LC-MS (Method 2): Rₜ = 1.24 min; MS (ESIpos): m/z = 391 [M + H]⁺ |
| | ¹H-NMR (400 MHz, DMSO-d₆): δ [ppm] = 1.03 (dd, 6H), 2.29-2.34 (m, 1H), |
| | 3.89 (s, 3H), 4.69 (dd, 1H), 7.15 (ddd, 1H), 7.28 (d, 1H), 7.37 (s, 1H), 7.43- |
| | 7.49 (m, 1H), 7.52 (t, 1H), 7.64 (d, 1H), 7.71-7.77 (m, 1H), 7.79 (s, 1H), |
| | 7.82 (dd, 1H), 7.89-7.94 (m, 1H), 8.32 (dd, 1H). |
| Example 104 | 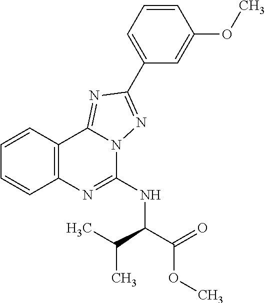 [SMILES: COC(=O)[C@H](Nc1nc2ccccc2c2nc(-c3cccc(OC)c3)nn12)C(C)C] |
| | methyl N-[2-(3-methoxyphenyl)[1,2,4]triazolo[1,5-c]quinazolin-5-yl]-D- |
| | valinate |
| | LC-MS (Method 2): Rₜ = 1.53 min; MS (ESIpos): m/z = 406 [M + H]⁺ |
| | ¹H-NMR (400 MHz, DMSO-d₆): δ [ppm] = 1.04 (d, 3H), 1.09 (d, 3H), 2.40- |
| | 2.48 (m, 1H), 3.71 (s, 3H), 3.90 (s, 3H), 4.66 (t, 1H), 7.15 (ddd, 1H), 7.47 |
| | (ddd, 1H), 7.52 (t, 1H), 7.61-7.65 (m, 1H), 7.70-7.77 (m, 1H), 7.86 (dd, |
| | 1H), 7.91-7.97 (m, 2H), 8.31 (dd, 1H). |
| Example 105 | 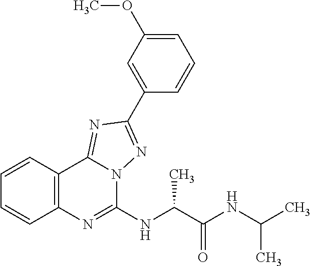 [SMILES: COc1cccc(-c2nc3c4ccccc4nc(N[C@H](C)C(=O)NC(C)C)n3n2)c1] |
| | N²-[2-(3-methoxyphenyl)[1,2,4]triazolo[1,5-c]quinazolin-5-yl]-N-propan- |
| | 2-yl-D-alaninamide |
| | LC-MS (Method 2): Rₜ = 1.27 min; MS (ESIpos): m/z = 405 [M + H]⁺ |
| | ¹H-NMR (400 MHz, DMSO-d₆): δ [ppm] = 1.08 (t, 6H), 1.52 (d, 3H), 3.85-3.95 |
| | (m, 4H), 4.72 (quin, 1H), 7.14 (ddd, 1H), 7.45 (ddd, 1H), 7.52 (t, 1H), 7.62 (d, |
| | 1H), 7.70-7.76 (m, 2H), 7.83 (dd, 1H), 7.91 (dt, 1H), 8.07 (d, 1H), 8.31 (dd, |
| | 1H). |
| Example 106 | 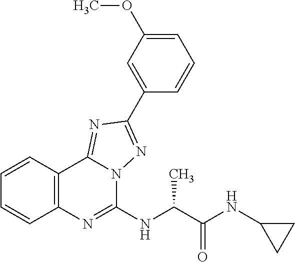 [SMILES: COc1cccc(-c2nc3c4ccccc4nc(N[C@H](C)C(=O)NC4CC4)n3n2)c1] |
| | N-cyclopropyl-N²-[2-(3-methoxyphenyl)[1,2,4]triazolo[1,5-c]quinazolin-5- |
| | yl]-D-alaninamide |
| | LC-MS (Method 2): Rₜ = 1.23 min; MS (ESIpos): m/z = 403 [M + H]⁺ |
| | ¹H-NMR (400 MHz, DMSO-d₆): δ [ppm] = 0.40-0.48 (m, 2H), 0.59-0.68 (m, |
| | 2H), 1.51 (d, 3H), 2.67 (td, 1H), 3.89 (s, 3H), 4.71 (quin, 1H), 7.14 (ddd, 1H), |
| | 7.45 (td, 1H), 7.52 (t, 1H), 7.61 (d, 1H), 7.70-7.76 (m, 1H), 7.79 (d, 1H), |
| | 7.84 (dd, 1H), 7.91 (dt, 1H), 8.24 (d, 1H), 8.31 (dd, 1H). |
| Example 107 | 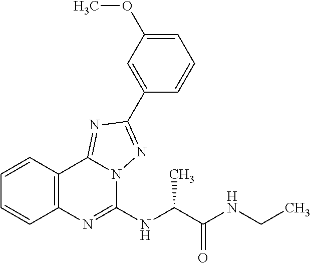 [SMILES: CCNC(=O)[C@@H](C)Nc1nc2ccccc2c2nc(-c3cccc(OC)c3)nn12] |
| | N-ethyl-N²-[2-(3-methoxyphenyl)[1,2,4]triazolo[1,5-c]quinazolin-5-yl]-D- |
| | alaninamide |
| | LC-MS (Method 2): Rₜ = 1.22 min; MS (ESIpos): m/z = 391 [M + H]⁺ |
| | ¹H-NMR (400 MHz, DMSO-d₆): δ [ppm] = 1.03 (t, 3H), 1.53 (d, 3H), 3.06-3.21 |
| | (m, 2H), 3.89 (s, 3H), 4.76 (quin, 1H), 7.13 (ddd, 1H), 7.44 (ddd, 1H), 7.50 (t, |
| | 1H), 7.61 (d, 1H), 7.68-7.76 (m, 1H), 7.79-7.85 (m, 2H), 7.91 (dt, 1H), 8.18 |
| | (t, 1H), 8.30 (dd, 1H). |
| Example 108 | 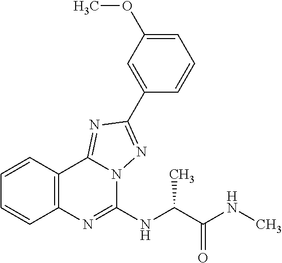 [SMILES: CNC(=O)[C@@H](C)Nc1nc2ccccc2c2nc(-c3cccc(OC)c3)nn12] |
| | N²-[2-(3-methoxyphenyl)[1,2,4]triazolo[1,5-c]quinazolin-5-yl]-N-methyl- |
| | D-alaninamide |
| | LC-MS (Method 2): Rₜ = 1.16 min; MS (ESIpos): m/z = 377 [M + H]⁺ |
| | ¹H-NMR (400 MHz, DMSO-d₆): δ [ppm] = 1.53 (d, 3H), 2.63 (d, 3H), 3.89 (s, |
| | 3H), 4.80 (quin, 1H), 7.14 (ddd, 1H), 7.44 (ddd, 1H), 7.51 (t, 1H), 7.63 (d, |
| | 1H), 7.70-7.75 (m, 1H), 7.84 (dd, 1H), 7.88-7.94 (m, 2H), 8.09 (q, 1H), |
| | 8.30 (dd, 1H). |
| Example 109 | 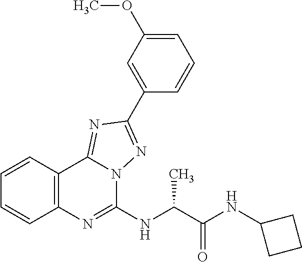 [SMILES: COc1cccc(-c2nc3c4ccccc4nc(N[C@H](C)C(=O)NC4CCC4)n3n2)c1] |
| | N-cyclobutyl-N²-[2-(3-methoxyphenyl)[1,2,4]triazolo[1,5-c]quinazolin-5- |
| | yl]-D-alaninamide |
| | LC-MS (Method 2): Rₜ = 1.36 min; MS (ESIpos): m/z = 417 [M + H]⁺ |
| | ¹H-NMR (400 MHz, DMSO-d₆): δ [ppm] = 1.51 (d, 3H), 1.57-1.67 (m, 2H), |
| | 1.85-2.00 (m, 2H), 2.09-2.25 (m, 2H), 3.89 (s, 3H), 4.16-4.30 (m, 1H), |
| | 4.74 (quin, 1H), 7.14 (ddd, 1H), 7.45 (td, 1H), 7.51 (t, 1H), 7.62 (d, 1H), 7.70- |
| | 7.76 (m, 1H), 7.78 (d, 1H), 7.84 (dd, 1H), 7.91 (dt, 1H), 8.31 (dd, 1H), 8.42 |
| | (d, 1H). |
| Example 110 | 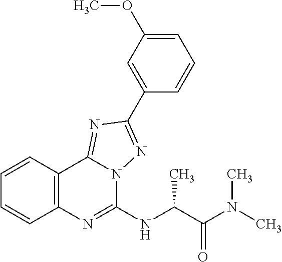 [SMILES: COc1cccc(-c2nc3c4ccccc4nc(N[C@H](C)C(=O)N(C)C)n3n2)c1] |
| | N²-[2-(3-methoxyphenyl)[1,2,4]triazolo[1,5-c]quinazolin-5-yl]-N,N- |
| | dimethyl-D-alaninamide |
| | LC-MS (Method 2): Rₜ = 1.33 min; MS (ESIpos): m/z = 391 [M + H]⁺ |
| | ¹H-NMR (400 MHz, DMSO-d₆): δ [ppm] = 1.51 (d, 3H), 2.91 (s, 3H), 3.21 (s, |
| | 3H), 3.89 (s, 3H), 5.19 (quin, 1H), 7.14 (ddd, 1H), 7.45 (ddd, 1H), 7.51 (t, |
| | 1H), 7.62-7.68 (m, 1H), 7.70-7.76 (m, 1H), 7.82-7.84 (m, 1H), 7.86-7.92 |
| | (m, 2H), 8.30 (dd, 1H). |
| Example 111 | 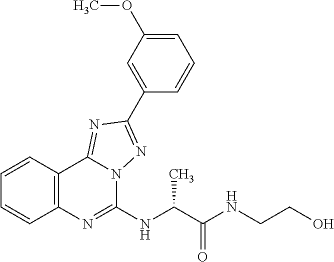 [SMILES: COc1cccc(-c2nc3c4ccccc4nc(N[C@H](C)C(=O)NCCO)n3n2)c1] |
| | N-(2-hydroxyethyl)-N²-[2-(3-methoxyphenyl)[1,2,4]triazolo[1,5- |
| | c]quinazolin-5-yl]-D-alaninamide |
| | LC-MS (Method 2): Rₜ = 1.07 min; MS (ESIpos): m/z = 407 [M + H]⁺ |
| | ¹H-NMR (400 MHz, DMSO-d₆): δ [ppm] = 1.53 (d, 3H), 3.09-3.27 (m, 2H), |
| | 3.41 (qd, 2H), 3.89 (s, 3H), 4.68 (t, 1H), 4.80 (quin, 1H), 7.14 (ddd, 1H), 7.45 |
| | (td, 1H), 7.52 (t, 1H), 7.63 (d, 1H), 7.71-7.76 (m, 1H), 7.82-7.88 (m, 2H), |
| | 7.92 (dt, 1H), 8.20 (t, 1H), 8.31 (dd, 1H). |
| Example 112 | 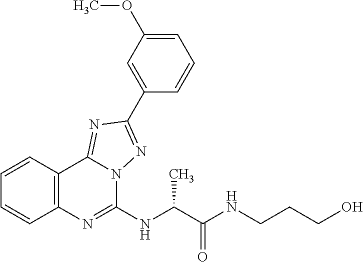 [SMILES: COc1cccc(-c2nc3c4ccccc4nc(N[C@H](C)C(=O)NCCCO)n3n2)c1] |
| | N-(3-hydroxypropyl)-N²-[2-(3-methoxyphenyl)[1,2,4]triazolo[1,5- |
| | c]quinazolin-5-yl]-D-alaninamide |
| | LC-MS (Method 2): Rₜ = 1.15 min; MS (ESIpos): m/z = 421 [M + H]⁺ |
| | ¹H-NMR (400 MHz, DMSO-d₆): δ [ppm] = 1.51-1.61 (m, 5H), 3.10-3.22 (m, |
| | 2H), 3.37-3.44 (m, 2H), 3.89 (s, 3H), 4.40 (t, 1H), 4.75 (quin, 1H), 7.14 (ddd, |
| | 1H), 7.45 (ddd, 1H), 7.52 (t, 1H), 7.62 (d, 1H), 7.70-7.76 (m, 1H), 7.81- |
| | 7.88 (m, 2H), 7.92 (dt, 1H), 8.17 (t, 1H), 8.31 (dd, 1H). |

### Example 113

### (2R)-2-{[2-(3-methoxyphenyl)[1,2,4]triazolo[1,5-c]quinazolin-5-yl]amino}-4-(methylsulfonyl)butanamide

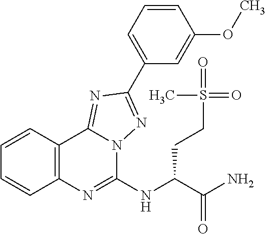 [SMILES: COc1cccc(-c2nc3c4ccccc4nc(N[C@H](CCS(C)(=O)=O)C(N)=O)n3n2)c1]

N²-[2-(3-Methoxyphenyl)[1,2,4]triazolo[1,5-c]quinazolin-5-yl]-D-methioninamide (example 96) (20.0 mg, 47.3 μmol) and oxone (18.0 mg, 118 μmol) were solubilsed in acetone (2.2 mL, 30 mmol)/water (900 μL) and the mixture was stirred overnight at rt. The mixture was diluted with water and the solid was filtered, washed with water and dried under reduced pressure at 60° C. to give 18.2 mg (90% purity, 76% yield) of the title compound.

LC-MS (method 2): Rₜ=1.03 min; MS (ESIpos): m/z=455 [M+H]⁺

¹H-NMR (400 MHz, DMSO-d₆): δ [ppm]=2.33-2.42 (m, 1H), 2.99 (s, 3H), 3.26 (dt, 2H), 3.89 (s, 3H), 4.88 (td, 1H), 7.15 (ddd, 1H), 7.40 (s, 1H), 7.46 (td, 1H), 7.52 (t, 1H), 7.64 (d, 1H), 7.74 (ddd, 2H), 7.84 (dd, 1H), 7.93 (dt, 1H), 8.00 (d, 1H), 8.32 (dd, 1H).

### Example 114

### N²-[2-(3-methoxyphenyl)[1,2,4]triazolo[1,5-c]quinazolin-5-yl]-L-lysinamide

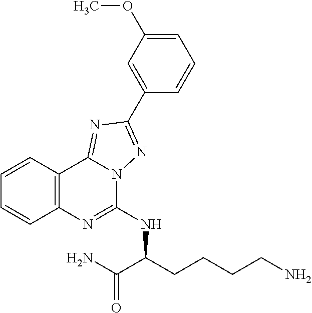 [SMILES: COc1cccc(-c2nc3c4ccccc4nc(N[C@@H](CCCCN)C(N)=O)n3n2)c1]

Tert-Butyl [(5S)-6-amino-5-{[2-(3-methoxyphenyl)[1,2,4]triazolo[1,5-c]quinazolin-5-yl]amino}-6-oxohexyl]carbamate (example 101) (97.1 mg, 187 μmol) was solubilised in 1,4-dioxane (2.0 mL) and HCl (400 μL, 4.0 M in dioxane, 1.6 mmol) was added. The mixture was stirred overnight at rt and 24 h at 60° C. The mixture was basified with sat. sodium hydrogen carbonate (pH 10), the organic solvent was evaporated and the suspension was filtered, washed with water and dried under reduced pressure at 60° C. to give 50.6 mg (90% purity, 58% yield) of the title compound.

LC-MS (Method 2): Rₜ=1.15 min; MS (ESIpos): m/z=420 [M+H]⁺

¹H-NMR (400 MHz, DMSO-d6): δ [ppm]=1.32-1.50 (m, 4H), 1.85-2.04 (m, 2H), 2.87-2.95 (m, 1H), 3.89 (s, 3H), 4.69-4.77 (m, 1H), 7.14 (dt, 1H), 7.26 (s, 1H), 7.44 (t, 1H), 7.51 (t, 1H), 7.63 (d, 1H), 7.66-7.76 (m, 2H), 7.81-7.86 (m, 1H), 7.92 (d, 1H), 8.30 (d, 1H).

### Example 115

### 6-{[2-(3-methoxyphenyl)[1,2,4]triazolo[1,5-c]quinazolin-5-yl]amino}-1,4-diazepan-5-one

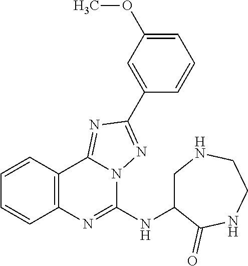 [SMILES: COc1cccc(-c2nc3c4ccccc4nc(NC4CNCCNC4=O)n3n2)c1]

Benzyl 6-{[2-(3-methoxyphenyl)[1,2,4]triazolo[1,5-c]quinazolin-5-yl]amino}-5-oxo-1,4-diazepane-1-carboxylate (example 172) (224 mg, 417 μmol) was solubilised with DMF (45 mL), and the reaction was placed under argon. Pd/C (44.3 mg) in DMF (1 mL) was added and the reaction mixture was placed under an atmosphere of hydrogen. The reaction was stirred for 2 h at rt and then filtered and concentrated under reduced pressure to provide 158 mg of the title compound (95% purity, 89% yield) without further purification.

LC-MS (method 2): Rₜ=1.04 min; MS (ESIpos): m/z=404 [M+H]⁺

¹H-NMR (400 MHz, DMSO-d6): δ [ppm]=3.03 (br dd, 1H), 3.07-3.15 (m, 1H), 3.41 (br dd, 2H), 3.89 (s, 3H), 4.89 (ddd, 1H), 7.15 (ddd, 1H), 7.43-7.55 (m, 2H), 7.66-7.81 (m, 4H), 7.88 (dt, 1H), 8.26-8.35 (m, 2H).

### Example 116

### 6-{[2-(3-methoxyphenyl)[1,2,4]triazolo[1,5-c]quinazolin-5-yl]amino}-1,4-diazepan-5-one, Enantiomer 1

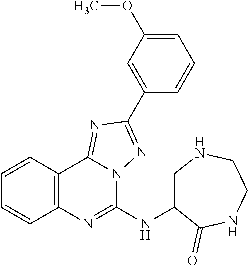 [SMILES: COc1cccc(-c2nc3c4ccccc4nc(NC4CNCCNC4=O)n3n2)c1]

Chiral HPLC separation of 6-{[2-(3-methoxyphenyl)[1,2,4]triazolo[1,5-c]quinazolin-5-yl]amino}-1,4-diazepan-5-one (example 115) was performed (Instrument: Labomatic HD5000, Labocord-5000; Gilson GX-241, Labcol Vario 4000, Column: Chiralpak IC 5μ 250×30 mm; Eluent: tert.-butylmethylether+0.1 vol-% diethylamine (99%)/ethanol 90:10; flow rate 50.0 mL/min; UV 254 nm).

Retention time of enantiomer 1: 2.02 min; [α]²⁰_{D}: +65° (c=1) in DMSO.

Instrument: Agilent HPLC 1260; Column: Chiralpak IC 3μ 100×4.6 mm; Eluent: tert.-butylmethylether+0.1 vol-% diethylamine (99%)/ethanol 90:10; flow rate 1.4 mL/min; temperature: 25° C.; DAD 254 nm.

### Example 117

### 6-{[2-(3-methoxyphenyl)[1,2,4]triazolo[1,5-c]quinazolin-5-yl]amino}-1,4-diazepan-5-one, Enantiomer 2

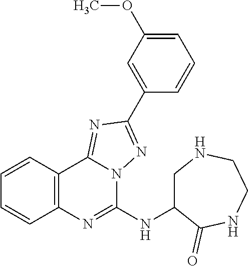 [SMILES: COc1cccc(-c2nc3c4ccccc4nc(NC4CNCCNC4=O)n3n2)c1]

The title compound was prepared as described for example 116.

Retention time of enantiomer 2: 3.24 min; [α]²⁰_{D}: −69° (c=1) in DMSO.

### Example 118

### 3-{[2-(3-methoxyphenyl)[1,2,4]triazolo[1,5-c]quinazolin-5-yl]amino}piperidin-2-one, Enantiomer 1

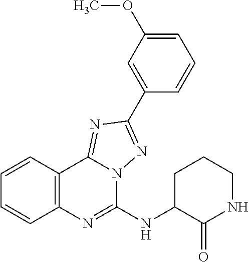 [SMILES: COc1cccc(-c2nc3c4ccccc4nc(NC4CCCNC4=O)n3n2)c1]

Chiral HPLC separation of 3-{[2-(3-methoxyphenyl)[1,2,4]triazolo[1,5-c]quinazolin-5-yl]amino}piperidin-2-one (example 77) was performed (Instrument: Sepiatec: Prep SFC100; Column: Chiralpak IG 5 μm 250×30 mm; Eluent A: CO2, Eluent B: methanol; isocratic: 57% B; flow rate 100.0 mL/min temperature: 40° C.; BPR: 150 bar; MWD @ 220 nm).

Retention time of enantiomer 1: 2.83 min; [α]²⁰_{D}: −31° (c=1) in DMSO.

Instrument: Agilent: 1260, Aurora SFC-Modul; Column: Chiralpak IG 5 μm 100×4.6 mm; Eluent A: CO2, Eluent B: methanol; isocratic: 57% B; flow rate 4.0 mL/min; temperature: 37.5° C.; BPR: 100 bar; MWD @ 220 nm.

### Example 119

### 3-{[2-(3-methoxyphenyl)[1,2,4]triazolo[1,5-c]quinazolin-5-yl]amino}piperidin-2-one, Enantiomer 2

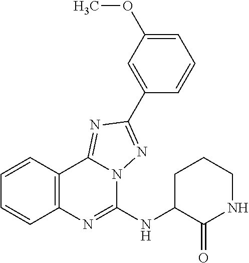 [SMILES: COc1cccc(-c2nc3c4ccccc4nc(NC4CCCNC4=O)n3n2)c1]

The title compound was prepared as described for example 118.

Retention time of enantiomer 2: 5.53 min; [α]²⁰_{D}: +33° (c=1) in DMSO.

### Example 120

### 3-{[2-(3-methoxyphenyl)[1,2,4]triazolo[1,5-c]quinazolin-5-yl]amino}pyrrolidin-2-one, Enantiomer 1

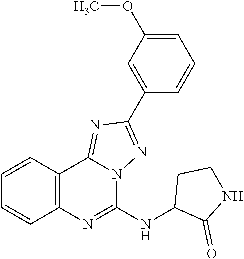 [SMILES: COc1cccc(-c2nc3c4ccccc4nc(NC4CCNC4=O)n3n2)c1]

Chiral HPLC separation of 3-{[2-(3-methoxyphenyl)[1,2,4]triazolo[1,5-c]quinazolin-5-yl]amino}pyrrolidin-2-one (example 78) was performed (Instrument: Sepiatec: Prep SFC100; Column: Chiralpak IB 5 μm 250×30 mm; Eluent A: CO2, Eluent B: methanol; isocratic: 38% B; flow rate 100.0 mL/min temperature: 40° C.; BPR: 150 bar; MWD @ 254 nm).

Retention time of enantiomer 1: 1.32 min; [α]²⁰_{D}: +5° (c=1) in DMSO.

Instrument: Agilent: 1260, Aurora SFC-Modul; Column: Chiralpak IB 5 μm 100×4.6 mm; Eluent A: CO2, Eluent B: methanol; isocratic: 38% B; flow rate 4.0 mL/min; temperature: 37.5° C.; BPR: 100 bar; MWD @ 254 nm.

### Example 121

### 3-{[2-(3-methoxyphenyl)[1,2,4]triazolo[1,5-c]quinazolin-5-yl]amino}pyrrolidin-2-one, Enantiomer 2

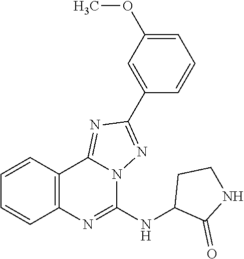 [SMILES: COc1cccc(-c2nc3c4ccccc4nc(NC4CCNC4=O)n3n2)c1]

The title compound was prepared as described for example 120.

Retention time of enantiomer 2: 1.73 min; [α]²⁰_{D}: −3° (c=1) in DMSO.

### Example 122

### 3-{[2-(3-methoxyphenyl)[1,2,4]triazolo[1,5-c]quinazolin-5-yl]amino}-1-methylpyrrolidin-2-one, Enantiomer 1

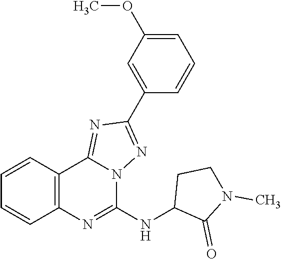 [SMILES: COc1cccc(-c2nc3c4ccccc4nc(NC4CCN(C)C4=O)n3n2)c1]

Chiral HPLC separation of 3-{[2-(3-methoxyphenyl)[1,2,4]triazolo[1,5-c]quinazolin-5-yl]amino}-1-methylpyrrolidin-2-one (example 80) was performed (Instrument: Labomatic HD5000, Labocord-5000; Gilson GX-241, Labcol Vario 4000, Column: YMC Amylose SA 5μ 250×30 mm; Eluent: tert.-butylmethylether/ethanol 85:15; flow rate 40.0 mL/min; UV 254 nm).

Retention time of enantiomer 1: 3.46 min; [α]²⁰_{D}: +21° (c=1) in DMSO.

Instrument: Agilent HPLC 1260; Column: YMC Amylose SA 3μ 100×4.6 mm; Eluent: tert.-Butylmethylether+0.1 vol-% diethylamine (99%)/ethanol 85:15; flow rate 1.4 mL/min; temperature: 25° C.; DAD 254 nm.

### Example 123

### 3-{[2-(3-methoxyphenyl)[1,2,4]triazolo[1,5-c]quinazolin-5-yl]amino}-1-methylpyrrolidin-2-one, Enantiomer 2

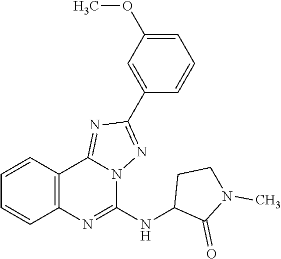 [SMILES: COc1cccc(-c2nc3c4ccccc4nc(NC4CCN(C)C4=O)n3n2)c1]

The title compound was prepared as described for example 122.

Retention time of enantiomer 2: 4.59 min; [α]²⁰_{D}: −15° (c=1) in DMSO.

### Example 124

### (3R)-3-{[2-(pyridin-2-yl)[1,2,4]triazolo[1,5-c]quinazolin-5-yl]amino}azepan-2-one

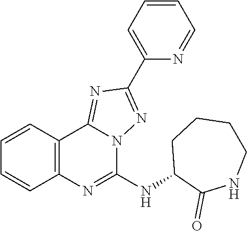 [SMILES: O=C1NCCCC[C@H]1Nc1nc2ccccc2c2nc(-c3ccccn3)nn12]

2-(Pyridin-2-yl)[1,2,4]triazolo[1,5-c]quinazoline-5(6H)-thione (100 mg, 358 μmol), (3R)-3-aminoazepan-2-one (138 mg, 1.07 mmol) and hydrogen peroxide (490 μL, 30% purity, 4.8 mmol) were stirred in DMSO (1.5 mL) at 80° C. for 4 h. The reaction mixture was diluted with water, filtered and the solid was washed with water. The solid was suspended in DMF (3 mL) and the reaction mixture was stirred at 80° C. for 2 hours. The reaction mixture was cooled to rt and the solid was filtered and washed with water. The solid material was dried under reduced pressure at 60° C. to give 17.5 mg (90% purity, 12% yield) of the title compound.

LC-MS (method 2): Rₜ=1.10 min; MS (ESIpos): m/z=374 [M+H]⁺

¹H-NMR (400 MHz, DMSO-d6): δ [ppm]=1.26-1.41 (m, 1H), 1.49-1.62 (m, 1H), 1.81-2.10 (m, 3H), 2.36 (br s, 1H), 3.11-3.24 (m, 1H), 4.85 (dd, 1H), 7.48 (td, 1H), 7.59 (ddd, 1H), 7.66-7.72 (m, 1H), 7.73-7.82 (m, 2H), 8.05 (td, 1H), 8.25 (dd, 1H), 8.33 (dd, 1H), 8.35-8.40 (m, 1H), 8.77-8.82 (m, 1H).

### Example 125

### (3R)-3-{[2-(pyridin-3-yl)[1,2,4]triazolo[1,5-c]quinazolin-5-yl]amino}azepan-2-one

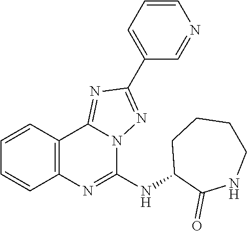 [SMILES: O=C1NCCCC[C@H]1Nc1nc2ccccc2c2nc(-c3cccnc3)nn12]

2-(Pyridin-3-yl)[1,2,4]triazolo[1,5-c]quinazoline-5(6H)-thione (100 mg, 358 μmol), (3R)-3-aminoazepan-2-one (138 mg, 1.07 mmol) and hydrogen peroxide (440 μL, 30% purity, 4.8 mmol) were stirred in DMSO (3.7 mL) at 80° C. for 4 h. The mixture was diluted with water, filtered, washed with water and dried under reduced pressure at 60° C. to give 38.2 mg (95% purity, 27% yield) of the title compound without further purification.

LC-MS (method 2): Rₜ=1.10 min; MS (ESIpos): m/z=374 [M+H]⁺

¹H-NMR (400 MHz, DMSO-d6): δ [ppm]=1.25-1.40 (m, 1H), 1.51-1.64 (m, 1H), 1.79-1.95 (m, 2H), 1.98-2.08 (m, 1H), 2.27-2.37 (m, 1H), 3.11-3.23 (m, 1H), 3.35-3.42 (m, 1H), 4.84 (dd, 1H), 7.43-7.50 (m, 1H), 7.60-7.70 (m, 2H), 7.72-7.81 (m, 2H), 8.22 (dd, 1H), 8.31 (dd, 1H), 8.59 (dt, 1H), 8.76 (dd, 1H), 9.42 (d, 1H).

### Example 126

### (3R)-3-{[2-(pyridin-4-yl)[1,2,4]triazolo[1,5-c]quinazolin-5-yl]amino}azepan-2-one

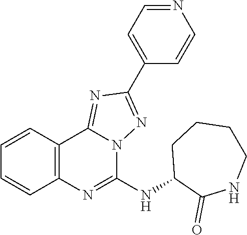 [SMILES: O=C1NCCCC[C@H]1Nc1nc2ccccc2c2nc(-c3ccncc3)nn12]

2-(Pyridin-4-yl)[1,2,4]triazolo[1,5-c]quinazoline-5(6H)-thione (362 mg, 1.30 mmol), (3R)-3-aminoazepan-2-one (498 mg, 3.89 mmol) and hydrogen peroxide (1.5 mL, 30% purity, 17 mmol) were stirred in DMSO (13 mL) at 80° C. for 4 h The mixture was diluted with water, filtered, washed with water and dried under reduced pressure at 60° C. The solid was suspended in DMSO (5 mL) und stirred at rt for 1 h. The suspension was filtered and the solid was washed with DMSO. The filtrate was purified by preparative HPLC to give 26.2 mg (97% purity, 5% yield) of the title compound.

LC-MS (Method 2): Rₜ=1.11 min; MS (ESIpos): m/z=374 [M+H]⁺

¹H-NMR (400 MHz, DMSO-d6): δ [ppm]=1.21-1.41 (m, 1H), 1.51-1.64 (m, 1H), 1.82-1.96 (m, 2H), 1.99-2.08 (m, 1H), 2.33 (td, 1H), 3.12-3.22 (m, 1H), 3.36 (br d, 1H), 4.85 (dd, 1H), 7.48 (ddd, 1H), 7.67-7.71 (m, 1H), 7.74-7.83 (m, 2H), 8.17-8.26 (m, 3H), 8.33 (dd, 1H), 8.80-8.85 (m, 2H).

### Example 127

### (3R)-3-{[2-(pyridazin-4-yl)[1,2,4]triazolo[1,5-c]quinazolin-5-yl]amino}azepan-2-one

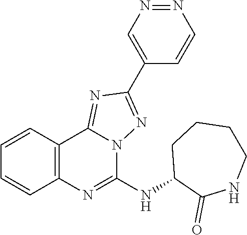 [SMILES: O=C1NCCCC[C@H]1Nc1nc2ccccc2c2nc(-c3ccnnc3)nn12]

2-(Pyridazin-4-yl)[1,2,4]triazolo[1,5-c]quinazoline-5(6H)-thione (50.0 mg, 67% purity, 120 μmol), (3R)-3-aminoazepan-2-one (46.0 mg, 359 μmol) and hydrogen peroxide (150 μl, 30% purity, 1.6 mmol) were stirred in DMSO (1.2 mL) at 80° C. for 4 h. The reaction mixture cooled to rt and diluted with water. The solid was filtered, washed with water and dried under reduced pressure at 60° C. to give 10.0 mg (95% purity, 21% yield) of the title compound.

LC-MS (method 2): Rₜ=1.01 min; MS (ESIneg): m/z=373 [M−H]⁻.

¹H-NMR (400 MHz, DMSO-d6): δ [ppm]=1.25-1.41 (m, 1H), 1.52-1.66 (m, 1H), 1.81-1.92 (m, 2H), 1.98-2.09 (m, 1H), 2.28-2.33 (m, 1H), 3.11-3.23 (m, 1H), 3.35-3.43 (m, 1H), 4.85 (dd, 1H), 7.49 (ddd, 1H), 7.66-7.73 (m, 1H), 7.74-7.81 (m, 1H), 7.84 (d, 1H), 8.23 (dd, 1H), 8.33 (dd, 1H), 8.41 (dd, 1H), 9.49 (dd, 1H), 9.96 (dd, 1H).

### Example 128

### (3R)-3-({2-[4-(dimethylamino)phenyl][1,2,4]triazolo[1,5-c]quinazolin-5-yl}amino)azepan-2-one

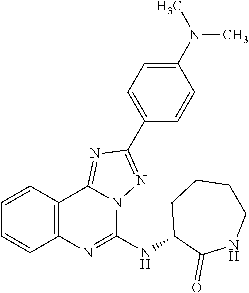 [SMILES: CN(C)c1ccc(-c2nc3c4ccccc4nc(N[C@@H]4CCCCNC4=O)n3n2)cc1]

2-[4-(Dimethylamino)phenyl][1,2,4]triazolo[1,5-c]quinazoline-5(6H)-thione (75.0 mg, 233 μmol), (3R)-3-aminoazepan-2-one (89.7 mg, 700 μmol) and hydrogen peroxide (290 μl, 33% purity, 3.1 mmol) were stirred in DMSO (2.4 mL) at 80° C. for 4 h. The reaction mixture was cooled to rt, diluted with water, filtered. The solid washed with water and dried under reduced pressure at 60° C. The solid was then solubilized in DMSO and purified by preparative HPLC to give 11.2 mg (100% purity, 12% yield) of the title compound.

LC-MS (method 2): Rₜ=1.36 min; MS (ESIpos): m/z=417 [M+H]⁺

¹H-NMR (400 MHz, DMSO-d₆): δ [ppm]=1.25-1.40 (m, 1H), 1.44-1.67 (m, 1H), 1.80-1.96 (m, 2H), 1.99-2.08 (m, 1H), 2.26-2.40 (m, 1H), 3.11-3.25 (m, 1H), 4.83 (dd, 1H), 6.79-6.92 (m, 2H), 7.43 (ddd, 1H), 7.60-7.78 (m, 3H), 8.05-8.15 (m, 2H), 8.20 (dd, 1H), 8.28 (dd, 1H).

### Example 129

### (3R)-3-{[2-(3-hydroxyphenyl)[1,2,4]triazolo[1,5-c]quinazolin-5-yl]amino}azepan-2-one

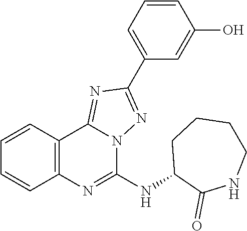 [SMILES: O=C1NCCCC[C@H]1Nc1nc2ccccc2c2nc(-c3cccc(O)c3)nn12]

(3R)-3-({2-[3-(Benzyloxy)phenyl][1,2,4]triazolo[1,5-c]quinazolin-5-yl}amino)azepan-2-one (200 mg, 418 μmol) was diluted with ethanol (5.0 mL), Pd/C (20 mg) was added and the mixture stirred for 5 h under an atmosphere of hydrogen at 50° C. Pd/C (20 mg) was again added and the mixture was stirred for 5 h under hydrogen at 50° C. Pd/C (20 mg) was again added and the mixture stirred for 5 h under hydrogen at 50° C. The reaction mixture was filtered and concentrated under reduced pressure. The crude mixture was solubilized in ethanol (5.0 mL) and Pd/C (50 mg) was added and the mixture stirred for 5 h under an atmosphere of hydrogen at 50° C. The mixture was then filtered and concentrated under reduced pressure. The crude mixture was purified by preparative HPLC (basic) to give 63.0 mg (98% purity, 38% yield) of the title compound.

LC-MS (method 2): Rₜ=1.04 min; MS (ESIpos): m/z=389 [M+H]⁺

¹H-NMR (400 MHz, DMSO-d6): δ [ppm]=1.24-1.39 (m, 1H), 1.48-1.62 (m, 1H), 1.80-1.95 (m, 2H), 1.97-2.06 (m, 1H), 2.28-2.36 (m, 1H), 3.11-3.22 (m, 1H), 3.32 (br d, 1H), 4.83 (dd, 1H), 6.90-6.97 (m, 1H), 7.35-7.42 (m, 1H), 7.46 (ddd, 1H), 7.65-7.69 (m, 1H), 7.71-7.77 (m, 4H), 8.22 (dd, 1H), 8.29 (dd, 1H), 9.80 (s, 1H).

### Example 130

### (3R)-3-{[2-(furan-2-yl)[1,2,4]triazolo[1,5-c]quinazolin-5-yl]amino}azepan-2-one

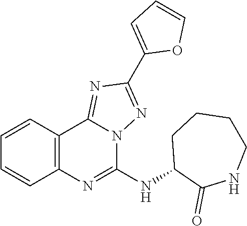 [SMILES: O=C1NCCCC[C@H]1Nc1nc2ccccc2c2nc(-c3ccco3)nn12]

5-Chloro-2-(furan-2-yl)[1,2,4]triazolo[1,5-c]quinazoline (75.0 mg, 277 μmol), (3R)-3-aminoazepan-2-one (53.3 mg, 416 μmol) and N,N-diisopropylethylamine (97 μL, 550 μmol) were stirred in DMF (1.2 mL) for 2 h at 60° C. The mixture was cooled to rt and the solid was filtered and washed with DMF. The solid and filtrate were combined, concentrated underreduced pressure and purified by preparative HPLC (basic) to give 61.3 mg (95% purity, 58% yield) of the title compound.

LC-MS (method 2): Rₜ=1.16 min; MS (ESIpos): m/z=363 [M+H]⁺

¹H-NMR (400 MHz, DMSO-d6): δ [ppm]=1.25-1.41 (m, 1H), 1.48-1.62 (m, 1H), 1.81-1.92 (m, 2H), 1.96-2.06 (m, 1H), 2.27-2.34 (m, 1H), 3.09-3.22 (m, 1H), 3.34-3.42 (m, 1H), 4.82 (dd, 1H), 6.73-6.78 (m, 1H), 7.32 (dd, 1H), 7.45 (ddd, 1H), 7.63-7.78 (m, 3H), 7.98 (dd, 1H), 8.18-8.30 (m, 2H).

### Example 131

### (3R)-3-{[2-(3-methyl-1,2,4-oxadiazol-5-yl)[1,2,4]triazolo[1,5-c]quinazolin-5-yl]amino}azepan-2-one

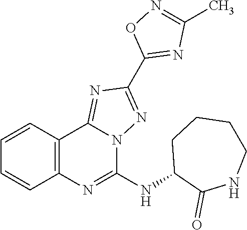 [SMILES: Cc1noc(-c2nc3c4ccccc4nc(N[C@@H]4CCCCNC4=O)n3n2)n1]

Ethyl 5-{[(3R)-2-oxoazepan-3-yl]amino}[1,2,4]triazolo[1,5-c]quinazoline-2-carboxylate intermediate 98 (68.9 mg, 187 μmol), N-hydroxyethanimidamide (32.8 mg, 95% purity, 421 μmol) and cesium carbonate (60.9 mg, 187 μmol) were stirred in 1,4-dioxane (2 mL) overnight at 110° C. The reaction mixture was then cooled to rt and diluted with water. The solid was filtered, washed with water and dried under reduced pressure to give 35.6 mg (99% purity, 50% yield) of the title compound without further purification.

LC-MS (method 2): Rₜ=1.10 min; MS (ESIpos): m/z=379 [M+H]⁺

¹H-NMR (400 MHz, DMSO-d6): δ [ppm]=1.26-1.39 (m, 1H), 1.50-1.63 (m, 1H), 1.80-1.95 (m, 2H), 1.98-2.08 (m, 1H), 2.29-2.39 (m, 1H), 2.53 (s, 3H), 3.11-3.22 (m, 1H), 3.34-3.43 (m, 1H), 4.83 (br dd, 1H), 7.51 (ddd, 1H), 7.69-7.74 (m, 1H), 7.77-7.83 (m, 1H), 7.86 (d, 1H), 8.26 (dd, 1H), 8.33 (dd, 1H).

### Example 132

### (3R)-3-{[2-(3-ethyl-1,2,4-oxadiazol-5-yl)[1,2,4]triazolo[1,5-c]quinazolin-5-yl]amino}azepan-2-one

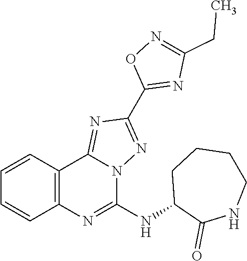 [SMILES: CCc1noc(-c2nc3c4ccccc4nc(N[C@@H]4CCCCNC4=O)n3n2)n1]

Ethyl 5-{[(3R)-2-oxoazepan-3-yl]amino}[1,2,4]triazolo[1,5-c]quinazoline-2-carboxylate intermediate 98 (75.0 mg, 204 μmol), N-hydroxypropanimidamide (40.4 mg, 458 μmol) and cesium carbonate (66.3 mg, 204 μmol) were stirred in 1,4-dioxane (2.0 mL) 5 h at 110° C. The reaction mixture was then cooled to rt and diluted with water. The solid was filtered, washed with water and dried under reduced pressure to give 18.0 mg (90% purity, 20% yield) of the title compound without further purification.

LC-MS (method 2): Rₜ=1.19 min; MS (ESIpos): m/z=393 [M+H]⁺

¹H-NMR (400 MHz, DMSO-d6): δ [ppm]=1.31 (br s, 1H), 1.35 (t, 3H), 1.50-1.64 (m, 1H), 1.81-1.95 (m, 2H), 1.97-2.09 (m, 1H), 2.29-2.37 (m, 1H), 2.91 (q, 2H), 3.11-3.23 (m, 1H), 3.35-3.43 (m, 1H), 4.76-4.89 (m, 1H), 7.47-7.54 (m, 1H), 7.68-7.74 (m, 1H), 7.77-7.84 (m, 1H), 7.86 (d, 1H), 8.26 (br dd, 1H), 8.34 (dd, 1H).

### Example 133

### (3R)-3-({2-[3-(propan-2-yl)-1,2,4-oxadiazol-5-yl][1,2,4]triazolo[1,5-c]quinazolin-5-yl}amino)azepan-2-one

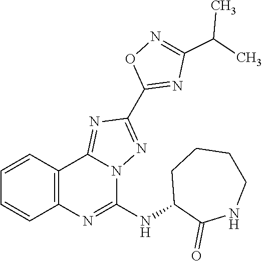 [SMILES: CC(C)c1noc(-c2nc3c4ccccc4nc(N[C@@H]4CCCCNC4=O)n3n2)n1]

Ethyl 5-{[(3R)-2-oxoazepan-3-yl]amino}[1,2,4]triazolo[1,5-c]quinazoline-2-carboxylate intermediate 98 (75.0 mg, 204 μmol), N-hydroxy-2-methylpropanimidamide (46.8 mg, 458 μmol) and cesium carbonate (66.3 mg, 204 μmol) were stirred in 1,4-dioxane (2.0 mL) overnight at 110° C. The reaction mixture was then cooled to rt and diluted with water. The solid was filtered, washed with water and dried under reduced pressure to give 36.0 mg (97% purity, 42% yield) of the title compound without further purification.

LC-MS (method 2): Rₜ=1.27 min; MS (ESIpos): m/z=407 [M+H]⁺

¹H-NMR (400 MHz, DMSO-d6): δ [ppm]=1.28-1.36 (m, 1H), 1.38 (d, 6H), 1.51-1.63 (m, 1H), 1.81-1.94 (m, 2H), 1.98-2.07 (m, 1H), 2.29-2.36 (m, 1H), 3.11-3.20 (m, 1H), 3.21-3.30 (m, 1H), 3.36-3.41 (m, 1H), 4.84 (br dd, 1H), 7.51 (ddd, 1H), 7.69-7.73 (m, 1H), 7.78-7.83 (m, 1H), 7.87 (d, 1H), 8.26 (dd, 1H), 8.35 (dd, 1H).

### Example 134

### (3R)-3-{[2-(3-tert-butyl-1,2,4-oxadiazol-5-yl)[1,2,4]triazolo[1,5-c]quinazolin-5-yl]amino}azepan-2-one

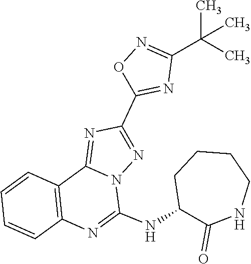 [SMILES: CC(C)(C)c1noc(-c2nc3c4ccccc4nc(N[C@@H]4CCCCNC4=O)n3n2)n1]

Ethyl 5-{[(3R)-2-oxoazepan-3-yl]amino}[1,2,4]triazolo[1,5-c]quinazoline-2-carboxylate intermediate 98 (75.0 mg, 204 μmol), N-hydroxy-2,2-dimethylpropanimidamide (53.2 mg, 458 μmol) and cesium carbonate (66.3 mg, 204 μmol) were stirred in 1,4-dioxane (2.0 mL) overnight at 110° C. The reaction mixture was then cooled to rt and diluted with water. The solid was filtered, washed with water and dried under reduced pressure to give 16.0 mg (100% purity, 19% yield) of the title compound without further purification.

LC-MS (Method 2): Rₜ=1.36 min; MS (ESIpos): m/z=421 [M+H]⁺

¹H-NMR (400 MHz, DMSO-d6): δ [ppm]=1.29-1.39 (m, 1H), 1.44 (s, 9H), 1.51-1.64 (m, 1H), 1.81-1.94 (m, 2H), 1.98-2.08 (m, 1H), 2.28-2.35 (m, 1H), 3.12-3.22 (m, 1H), 3.40 (br d, 1H), 4.84 (br dd, 1H), 7.51 (ddd, 1H), 7.69-7.74 (m, 1H), 7.78-7.83 (m, 1H), 7.87 (d, 1H), 8.25 (dd, 1H), 8.35 (dd, 1H).

### Example 135

### (3R)-3-{[2-(3-cyclopropyl-1,2,4-oxadiazol-5-yl)[1,2,4]triazolo[1,5-c]quinazolin-5-yl]amino}azepan-2-one

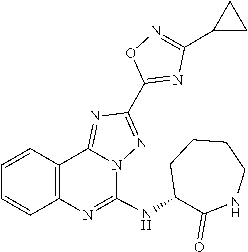 [SMILES: O=C1NCCCC[C@H]1Nc1nc2ccccc2c2nc(-c3nc(C4CC4)no3)nn12]

Ethyl 5-{[(3R)-2-oxoazepan-3-yl]amino}[1,2,4]triazolo[1,5-c]quinazoline-2-carboxylate intermediate 98 (75.0 mg, 204 μmol), N-hydroxycyclopropanecarboximidamide (45.9 mg, 458 μmol) and cesium carbonate (66.3 mg, 204 μmol) were stirred in 1,4-dioxane (2.0 mL) overnight at 110° C. The reaction mixture was then cooled to rt and diluted with water. The solid was filtered, washed with water and dried under reduced pressure to give 43.0 mg (96% purity, 50% yield) of the title compound without further purification.

LC-MS (method 2): Rₜ=1.22 min; MS (ESIpos): m/z=405 [M+H]⁺

¹H-NMR (400 MHz, DMSO-d6): δ [ppm]=1.04-1.11 (m, 2H), 1.17-1.21 (m, 2H), 1.26-1.39 (m, 1H), 1.50-1.63 (m, 1H), 1.80-1.94 (m, 2H), 1.97-2.08 (m, 1H), 2.27-2.36 (m, 2H), 3.11-3.22 (m, 1H), 3.36-3.42 (m, 1H), 4.83 (br dd, 1H), 7.50 (ddd, 1H), 7.71 (d, 1H), 7.77-7.83 (m, 1H), 7.84 (d, 1H), 8.25 (dd, 1H), 8.33 (dd, 1H).

### Example 136

### (3R)-3-({2-[3-(trifluoromethyl)-1,2,4-oxadiazol-5-yl][1,2,4]triazolo[1,5-c]quinazolin-5-yl}amino)azepan-2-one

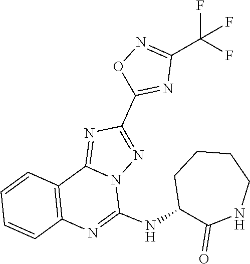 [SMILES: O=C1NCCCC[C@H]1Nc1nc2ccccc2c2nc(-c3nc(C(F)(F)F)no3)nn12]

Ethyl 5-{[(3R)-2-oxoazepan-3-yl]amino}[1,2,4]triazolo[1,5-c]quinazoline-2-carboxylate intermediate 98 (75.0 mg, 204 μmol), trifluoro-N-hydroxyethanimidamide (58.7 mg, 458 μmol) and cesium carbonate (66.3 mg, 204 μmol) were stirred in 1,4-dioxane (2.0 mL) overnight at 110° C. The reaction mixture was then cooled to rt and diluted with water. The solid was filtered, washed with water and dried under reduced pressure to give 24.0 mg (98% purity, 27% yield) of the title compound without further purification.

LC-MS (method 2): Rₜ=1.31 min; MS (ESIpos): m/z=433 [M+H]⁺

¹H-NMR (400 MHz, DMSO-d6): δ [ppm]=1.29-1.40 (m, 1H), 1.53-1.66 (m, 1H), 1.81-1.94 (m, 2H), 1.98-2.08 (m, 1H), 2.29-2.36 (m, 1H), 3.11-3.23 (m, 1H), 3.40 (br d, 1H), 4.85 (br dd, 1H), 7.53 (ddd, 1H), 7.73 (d, 1H), 7.79-7.86 (m, 1H), 7.94 (d, 1H), 8.26 (dd, 1H), 8.36 (dd, 1H).

The following examples were prepared analogously to example 1 starting from the desired intermediate:

| | Structure |
|---|---|
| | IUPAC-Name |
| | LC-MS (Method): Retention time; Mass found |
| Example | ¹H-NMR |
| Example 137 | 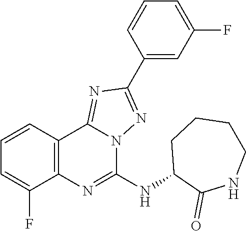 [SMILES: O=C1NCCCC[C@H]1Nc1nc2c(F)cccc2c2nc(-c3cccc(F)c3)nn12] |
| | (3R)-3-{[7-fluoro-2-(3-fluorophenyl)[1,2,4]triazolo[1,5-c]quinazolin-5- |
| | yl]amino}azepan-2-one |
| | LC-MS (Method 1): Rₜ = 1.38 min; MS (ESIneg): m/z = 407 [M − H]⁻ |
| | ¹H-NMR (400 MHz, DMSO-d₆): δ [ppm] = 1.24-1.40 (m, 1H), 1.50-1.64 (m, |
| | 1H), 1.76-1.91 (m, 2H), 1.97-2.09 (m, 1H), 2.31-2.39 (m, 1H), 3.11-3.23 |
| | (m, 1H), 3.29-3.34 (m, 1H), 4.83 (dd, 1H), 7.39-7.47 (m, 2H), 7.58-7.70 |
| | (m, 2H), 7.90 (d, 1H), 7.96-8.02 (m, 1H), 8.09-8.15 (m, 2H), 8.23 (dd, 1H). |
| Example 138 | 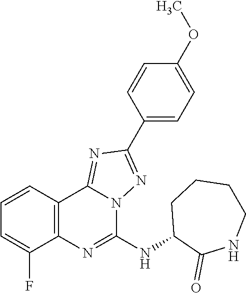 [SMILES: COc1ccc(-c2nc3c4cccc(F)c4nc(N[C@@H]4CCCCNC4=O)n3n2)cc1] |
| | (3R)-3-{[7-fluoro-2-(4-methoxyphenyl)[1,2,4]triazolo[1,5-c]quinazolin-5- |
| | yl]amino}azepan-2-one |
| | LC-MS (Method 2): Rₜ = 1.33 min; MS (ESIpos): m/z = 421 [M + H]⁺ |
| | ¹H-NMR (400 MHz, DMSO-d₆): δ [ppm] = 1.25-1.40 (m, 1H), 1.50-1.64 (m, |
| | 1H), 1.77-1.92 (m, 2H), 1.98-2.08 (m, 1H), 2.31-2.39 (m, 1H), 3.10-3.21 |
| | (m, 1H), 3.37-3.41 (m, 1H), 3.86 (s, 3H), 4.83 (dd, 1H), 7.11-7.17 (m, 2H), |
| | 7.41 (td, 1H), 7.60 (ddd, 1H), 7.84 (d, 1H), 8.07-8.12 (m, 1H), 8.18-8.25 |
| | (m, 3H). |
| Example 139 | 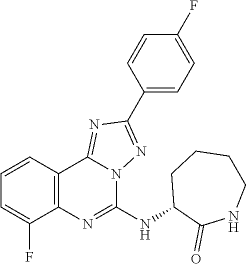 [SMILES: O=C1NCCCC[C@H]1Nc1nc2c(F)cccc2c2nc(-c3ccc(F)cc3)nn12] |
| | (3R)-3-{[7-fluoro-2-(4-fluorophenyl)[1,2,4]triazolo[1,5-c]quinazolin-5- |
| | yl]amino}azepan-2-one |
| | LC-MS (Method 2): Rₜ = 1.37 min; MS (ESIpos): m/z = 409 [M + H]⁺ |
| | ¹H-NMR (400 MHz, DMSO-d₆): δ [ppm] = 1.25-1.40 (m, 1H), 1.50-1.63 (m, |
| | 1H), 1.78-1.92 (m, 2H), 1.97-2.09 (m, 1H), 2.31-2.39 (m, 1H), 3.10-3.22 |
| | (m, 1H), 3.27-3.33 (m, 1H), 4.83 (dd, 1H), 7.39-7.47 (m, 3H), 7.61 (ddd, |
| | 1H), 7.87 (d, 1H), 8.10 (dd, 1H), 8.23 (dd, 1H), 8.29-8.36 (m, 2H). |
| Example 140 | 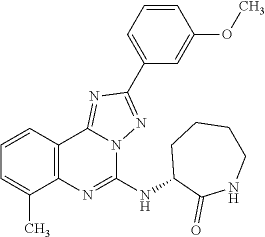 [SMILES: COc1cccc(-c2nc3c4cccc(C)c4nc(N[C@@H]4CCCCNC4=O)n3n2)c1] |
| | (3R)-3-{[2-(3-methoxyphenyl)-7-methyl[1,2,4]triazolo[1,5-c]quinazolin-5- |
| | yl]amino}azepan-2-one |
| | LC-MS (Method 2): Rₜ = 1.46 min; MS (ESIpos): m/z = 417 [M + H]⁺ |
| | ¹H-NMR (400 MHz, DMSO-d₆): δ [ppm] = 1.27-1.40 (m, 1H), 1.50-1.62 (m, |
| | 1H), 1.80-1.94 (m, 2H), 2.01-2.11 (m, 1H), 2.40 (br d, 1H), 2.61 (s, 3H), |
| | 3.13-3.23 (m, 1H), 3.34-3.41 (m, 1H), 3.89 (s, 3H), 4.83 (dd, 1H), 7.14 |
| | (ddd, 1H), 7.35 (t, 1H), 7.51 (t, 1H), 7.63 (dt, 1H), 7.67 9d, 1H), 7.78 (dd, 1H), |
| | 7.87 (dt, 1H), 8.13-8.18 (m, 1H), 8.22 (dd, 1H). |
| Example 141 | 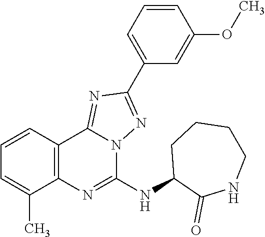 [SMILES: COc1cccc(-c2nc3c4cccc(C)c4nc(N[C@H]4CCCCNC4=O)n3n2)c1] |
| | (3S)-3-{[2-(3-methoxyphenyl-7-methyl[1,2,4]triazolo[1,5-c]quinazolin-5- |
| | yl]amino}azepan-2-one |
| | LC-MS (Method 2): Rₜ = 1.46 min; MS (ESIpos): m/z = 417 [M + H]⁺ |
| | ¹H-NMR (400 MHz, DMSO-d₆): δ [ppm] = 1.27-1.41 (m, 1H), 1.50-1.62 (m |
| | 1H), 1.80-1.94 (m, 2H), 2.02-2.10 (m, 1H), 2.40 (br d, 1H), 2.61 (s, 3H), |
| | 3.19 (br dd, 1H), 3.34-3.41 (m, 1H), 3.89 (s, 3H), 4.84 (dd, 1H), 7.14 (ddd, |
| | 1H), 7.35 (t, 1H), 7.51 (t, 1H), 7.63 (dt, 1H), 7.67 (d, 1H), 7.78 (dd, 1H), 7.88 |
| | (dt, 1H), 8.15 (dd, 1H), 8.22 (dd, 1H). |
| Example 142 | 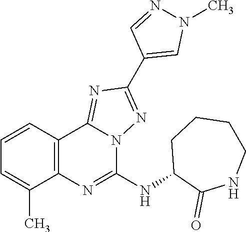 [SMILES: Cc1cccc2c1nc(N[C@@H]1CCCCNC1=O)n1nc(-c3cnn(C)c3)nc21] |
| | (3R)-3-{[7-methyl-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.18 min; MS (ESIpos): m/z = 391 [M + H]⁺ |
| | ¹H-NMR (400 MHz, DMSO-d₆): δ [ppm] = 1.26-1.41 (m, 1H), 1.52 (q, 1H), |
| | 1.87 (br d, 2H), 1.99-2.11 (m, 1H), 2.40 (br d, 1H), 2.61 (s, 3H), 3.11-3.23 |
| | (m, 1H), 3.95 (s, 3H), 4.82 (br dd, 1H), 7.33 (t, 1H), 7.54 (d, 1H), 7.62 (br d, |
| | 1H), 8.04-8.12 (m, 2H), 8.22 (br t, 1H), 8.49 (s, 1H). |
| Example 143 | 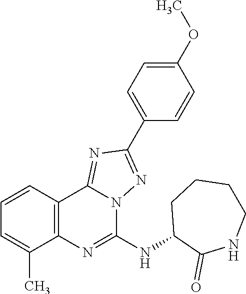 [SMILES: COc1ccc(-c2nc3c4cccc(C)c4nc(N[C@@H]4CCCCNC4=O)n3n2)cc1] |
| | (3R)-3-{[2-(4-methoxyphenyl-7-methyl[1,2,4]triazolo[1,5-c]quinazolin-5- |
| | yl]amino}azepan-2-one |
| | LC-MS (Method 2): Rₜ = 1.45 min; MS (ESIpos): m/z = 417 [M + H]⁺ |
| | ¹H-NMR (400 MHz, DMSO-d₆): δ [ppm] = 1.27-1.40 (m, 1H), 1.48-1.61 (m, |
| | 1H), 1.80-1.94 (m, 2H), 2.02-2.10 (m, 1H), 2.41 (br d, 1H), 2.61 (s, 3H), |
| | 3.13-3.22 (m, 1H), 3.35-3.41 (m, 1H), 3.86 (s, 3H), 4.83 (dd, 1H), 7.11- |
| | 7.16 (m, 2H), 7.34 (t, 1H), 7.59-7.66 (m, 2H), 8.14 (d, 1H), 8.18-8.26 (m, |
| | 3H). |
| Example 144 | 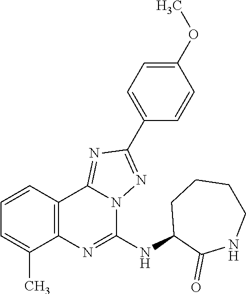 [SMILES: COc1ccc(-c2nc3c4cccc(C)c4nc(N[C@H]4CCCCNC4=O)n3n2)cc1] |
| | (3S)-2-{[2-(4-methoxyphenyl)-7-methyl[1,2,4]triazolo[1,5-c]quinazolin-5- |
| | yl]amino}azepan-2-one |
| | LC-MS (Method 2): Rₜ = 1.45 min; MS (ESIpos): m/z = 417 [M + H]⁺ |
| | ¹H-NMR (400 MHz, DMSO-d₆): δ [ppm] = 1.27-1.41 (m, 1H), 1.48-1.61 (m, |
| | 1H), 1.81-1.94 (m, 2H), 2.01-2.10 (m, 1H), 2.41 (br d, 1H), 2.61 (s, 3H), |
| | 3.13-3.23 (m, 1H), 3.35-3.41 (m, 1H), 3.86 (s, 3H), 4.83 (dd, 1H), 7.11- |
| | 7.17 (m, 2H), 7.34 (t, 1H), 7.60-7.66 (m, 2H), 8.14 (d, 1H), 8.19-8.26 (m, |
| | 3H). |
| Example 145 | 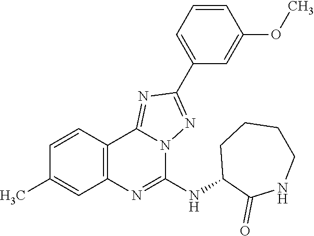 [SMILES: COc1cccc(-c2nc3c4ccc(C)cc4nc(N[C@@H]4CCCCNC4=O)n3n2)c1] |
| | (3R)-3-{[2-(3-methoxyphenyl)-8-methyl[1,2,4]triazolo[1,5-c]quinazolin-5- |
| | yl]amino}azepan-2-one |
| | LC-MS (Method 2): Rₜ = 1.39 min; MS (ESIpos): m/z = 417 [M + H]⁺ |
| | ¹H-NMR (400 MHz, DMSO-d₆): δ [ppm] = 1.26-1.39 (m, 1H), 1.51-1.63 (m, |
| | 1H), 1.81-1.94 (m, 2H), 1.98-2.07 (m, 1H), 2.27-2.35 (m, 1H), 3.12-3.23 |
| | (m, 1H), 3.89 (s, 3H), 4.82 (dd, 1H), 7.14 (ddd, 1H), 7.29 (dd, 1H), 7.48- |
| | 7.54 (m, 2H), 7.71 (d, 1H), 7.78 (dd, 1H), 7.87 (dt, 1H), 8.17-8.23 (m, 2H). |
| Example 146 | 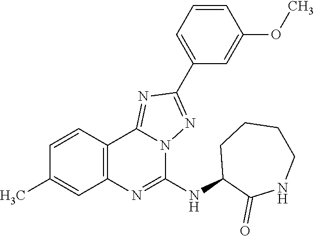 [SMILES: COc1cccc(-c2nc3c4ccc(C)cc4nc(N[C@H]4CCCCNC4=O)n3n2)c1] |
| | (3S)-3-{[2-(3-methoxyphenyl)-8-methyl[1,2,4]triazolo[1,5-c]quinazolin-5- |
| | yl]amino}azepan-2-one |
| | LC-MS (Method 2): Rₜ = 1.39 min; MS (ESIpos): m/z = 417 [M + H]⁺ |
| | ¹H-NMR (400 MHz, DMSO-d₆): δ [ppm] = 1.25-1.39 (m, 1H), 1.51-1.63 (m, |
| | 1H), 1.80-1.94 (m, 2H), 1.98-2.07 (m, 1H), 2.27-2.35 (m, 1H), 3.11-3.22 |
| | (m, 1H), 3.89 (s, 3H), 4.82 (br dd, 1H), 7.14 (dd, 1H), 7.27-7.31 (m, 1H), |
| | 7.47-7.54 (m, 2H), 7.71 (d, 1H), 7.76-7.79 (m, 1H), 7.87 (d, 1H), 8.17- |
| | 8.24 (m, 2H). |
| Example 147 | 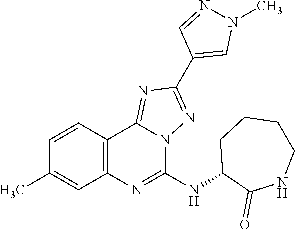 [SMILES: Cc1ccc2c(c1)nc(N[C@@H]1CCCCNC1=O)n1nc(-c3cnn(C)c3)nc21] |
| | (3R)-3-{[8-methyl-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.10 min; MS (ESIpos): m/z = 391 [M + H]⁺ |
| | ¹H-NMR (400 MHz, DMSO-d₆): δ [ppm] = 1.24-1.43 (m, 1H), 1.46-1.62 (m, |
| | 1H), 1.78-1.94 (m, 2H), 1.95-2.08 (m, 1H), 2.28 (br s, 1H), 3.08-3.24 (m, |
| | 1H), 3.95 (s, 3H), 4.80 (br dd, 1H), 7.27 (dd, 1H), 7.48 (s, 1H), 7.56 (d, 1H), |
| | 8.05 (d, 1H), 8.12 (d, 1H), 8.21 (dd, 1H), 8.47 (s, 1H). |
| Example 148 | 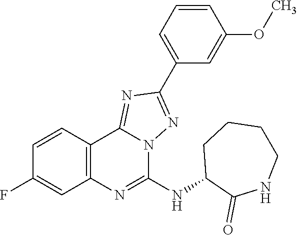 [SMILES: COc1cccc(-c2nc3c4ccc(F)cc4nc(N[C@@H]4CCCCNC4=O)n3n2)c1] |
| | (3R)-3-{[8-fluoro-2-(3-methoxyphenyl)[1,2,4]triazolo[1,5-c]quinazolin-5- |
| | yl]amino}azepan-2-one |
| | LC-MS (Method 2): Rₜ = 1.36 min; MS (ESIpos): m/z = 421 [M + H]⁺ |
| | ¹H-NMR (400 MHz, DMSO-d₆): δ [ppm] = 1.25-1.40 (m, 1H), 1.51-1.67 (m, |
| | 1H), 1.80-1.94 (m, 2H), 1.98-2.07 (m, 1H), 2.28 (br s, 1H), 3.11-3.23 (m, |
| | 1H), 3.36-3.42 (m, 1H), 3.89 (s, 3H), 4.83 (dd, 1H), 7.15 (ddd, 1H), 7.32 (td, |
| | 1H), 7.41 (dd, 1H), 7.52 (t, 1H), 7.78 (dd, 1H), 7.85-7.90 (m, 2H), 8.22 (dd, |
| | 1H), 8.36 (dd, 1H). |
| Example 149 | 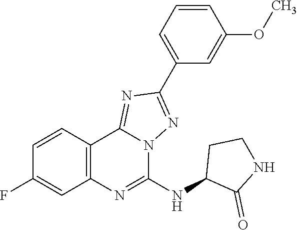 [SMILES: COc1cccc(-c2nc3c4ccc(F)cc4nc(N[C@H]4CCNC4=O)n3n2)c1] |
| | (3S)-3-{[8-fluoro-2-(3-methoxyphenyl)[1,2,4]triazolo[1,5-c]quinazolin-5- |
| | yl]amino}pyrrolidin-2-one |
| | LC-MS (Method 2): Rₜ = 1.15 min; MS (ESIpos): m/z = 393 [M + H]⁺ |
| | ¹H-NMR (400 MHz, DMSO-d₆): δ [ppm] = 2.25-2.39 (m, 1H), 4.92-5.02 (m, |
| | 1H), 7.14 (ddd, 1H), 7.31 (td, 1H), 7.37 (dd, 1H), 7.52 (t, 1H), 7.83 (dd, 1H), |
| | 7.90 (dt, 1H), 8.02 (s, 1H), 8.35 (dd, 1H), 8.51 (d, 1H). |
| Example 150 | 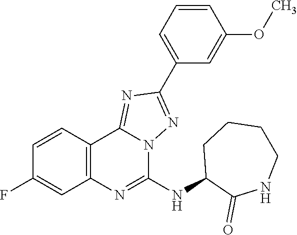 [SMILES: COc1cccc(-c2nc3c4ccc(F)cc4nc(N[C@H]4CCCCNC4=O)n3n2)c1] |
| | (3S)-3-{[8-fluoro-2-(3-methoxyphenyl)[1,2,4]triazolo[1,5-c]quinazolin-5- |
| | yl]amino}azepan-2-one |
| | LC-MS (Method 2): Rₜ = 1.36 min; MS (ESIpos): m/z = 421 [M + H]⁺ |
| Example 151 | 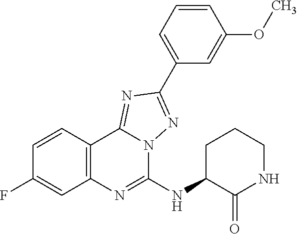 [SMILES: COc1cccc(-c2nc3c4ccc(F)cc4nc(N[C@H]4CCCNC4=O)n3n2)c1] |
| | (3S)-3-{[8-fluoro-2-(3-methoxyphenyl)[1,2,4]triazolo[1,5-c]quinazolin-5- |
| | yl]amino}piperidin-2-one |
| | LC-MS (Method 2): Rₜ = 1.25 min; MS (ESIpos): m/z = 407 [M + H]⁺ |
| | ¹H-NMR (400 MHz, DMSO-d₆): δ [ppm] = 1.84-1.98 (m, 2H), 2.04-2.18 (m, |
| | 1H), 2.21-2.30 (m, 1H), 3.19-3.29 (m, 2H), 3.89 (s, 3H), 4.68-4.77 (m, |
| | 1H), 7.14 (ddd, 1H), 7.30 (td, 1H), 7.39 (dd, 1H), 7.52 (t, 1H), 7.82 (dd, 2H), |
| | 7.89 (dt, 1H), 8.32-8.41 (m, 2H). |
| Example 152 | 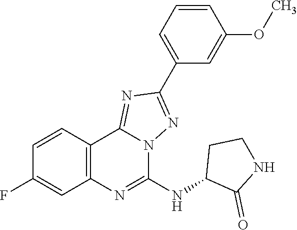 [SMILES: COc1cccc(-c2nc3c4ccc(F)cc4nc(N[C@@H]4CCNC4=O)n3n2)c1] |
| | (3R)-3-{[8-fluoro-2-(3-methoxyphenyl)[1,2,4]triazolo[1,5-c]quinazolin-5- |
| | yl]amino}pyrrolidin-2-one |
| | LC-MS (Method 2): Rₜ = 1.15 min; MS (ESIpos): m/z = 393 [M + H]⁺ |
| Example 153 | 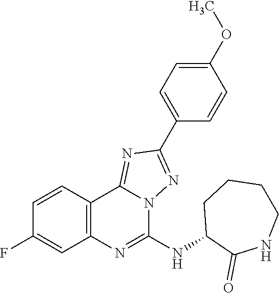 [SMILES: COc1ccc(-c2nc3c4ccc(F)cc4nc(N[C@@H]4CCCCNC4=O)n3n2)cc1] |
| | (3R)-3-{[8-fluoro-2-(4-methoxyphenyl)[1,2,4]triazolo[1,5-c]quinazolin-5- |
| | yl]amino}azepan-2-one |
| | LC-MS (Method 2): Rₜ = 1.35 min; MS (ESIpos): m/z = 421 [M + H]⁺ |
| | ¹H-NMR (400 MHz, DMSO-d₆): δ [ppm] = 1.25-1.42 (m, 1H), 1.49-1.65 (m, |
| | 1H), 1.80-1.94 (m, 2H), 1.97-2.08 (m, 1H), 2.26-2.35 (m, 1H), 2.54 (s, |
| | 1H), 3.10-3.24 (m, 1H), 3.86 (s, 3H), 4.82 (dd, 1H), 7.09-7.20 (m, 2H), |
| | 7.31 (td, 1H), 7.40 (dd, 1H), 7.82 (d, 1H), 8.17-8.26 (m, 3H), 8.33 (dd, 1H). |
| Example 154 | 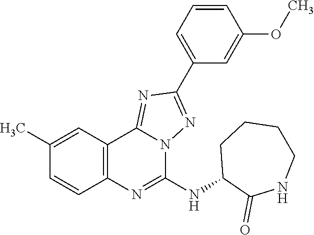 [SMILES: COc1cccc(-c2nc3c4cc(C)ccc4nc(N[C@@H]4CCCCNC4=O)n3n2)c1] |
| | (3R)-3-{[2-(3-methoxyphenyl)-9-methyl[1,2,4]triazolo[1,5-c]quinazolin-5- |
| | yl]amino}azepan-2-one |
| | LC-MS (Method 2): Rₜ = 1.40 min; MS (ESIneg): m/z = 415 [M − H]⁻ |
| | ¹H-NMR (400 MHz, DMSO-d₆): δ [ppm] = 1.26-1.39 (m, 1H), 1.51-1.63 (m, |
| | 1H), 1.80-1.91 (m, 2H), 1.98-2.07 (m, 1H), 2.27-2.34 (m, 1H), 3.12-3.21 |
| | (m, 1H), 3.34-3.41 (m, 1H), 3.89 (s, 3H), 4.82 (dd, 1H), 7.14 (ddd, 1H), 7.51 |
| | (t, 1H), 7.56-7.58 (m, 2H), 7.67 (d, 1H), 7.78 (dd, 1H), 7.87 (dt, 1H), 8.10- |
| | 8.13 (m, 1H), 8.20 (dd, 1H). |
| Example 155 | 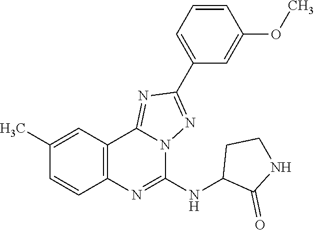 |
| | 3-{[2-(3-methoxyphenyl)-9-methyl[1,2,4]triazolo[1,5-c]quinazolin-5- |
| | yl]amino}pyrrolidin-2-one |
| | LC-MS (Method 2): Rₜ = 1.20 min; MS (ESIpos): m/z = 389 [M + H]⁺ |
| | ¹H-NMR (400 MHz, DMSO-d₆): δ [ppm] = 2.24-2.38 (m, 1H), 2.54 (s, 1H), |
| | 3.89 (s, 3H), 4.93 (dt, 1H), 7.14 (ddd, 1H), 7.48-7.58 (m, 3H), 7.83 (dd, 1H), |
| | 7.90 (dt, 1H), 8.00 (s, 1H), 8.09-8.13 (m, 1H), 8.21 (d, 1H). |
| Example 156 | 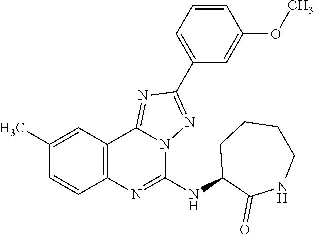 [SMILES: COc1cccc(-c2nc3c4cc(C)ccc4nc(N[C@H]4CCCCNC4=O)n3n2)c1] |
| | (3S)-3-{[2-(3-methoxyphenyl)-9-methyl[1,2,4]triazolo[1,5-c]quinazolin-5- |
| | yl]amino}azepan-2-one |
| | LC-MS (Method 2): Rₜ = 1.40 min; MS (ESIpos): m/z = 417 [M + H]⁺ |
| Example 157 | 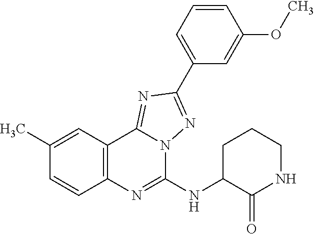 |
| | 3-{[2-(3-methoxyphenyl)-9-methyl[1,2,4]triazolo[1,5-c]quinazolin-5- |
| | yl]amino}piperidin-2-one |
| | LC-MS (Method 2): Rₜ = 1.27 min; MS (ESIneg): m/z = 401 [M − H]⁻ |
| | ¹H-NMR (400 MHz, DMSO-d₆): δ [ppm] = 1.85-1.98 (m, 2H), 2.09 (qd, 1H), |
| | 2.24-2.34 (m, 1H), 3.21-3.29 (m, 2H), 3.89 (s, 3H), 4.69 (dt, 1H), 7.14 |
| | (ddd, 1H), 7.52 (t, 1H), 7.54-7.56 (m, 2H), 7.80 (br s, 1H), 7.82 (dd, 1H), |
| | 7.86-7.91 (m, 1H), 8.06-8.12 (m, 2H). |
| Example 158 | 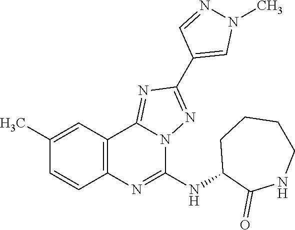 [SMILES: Cc1ccc2nc(N[C@@H]3CCCCNC3=O)n3nc(-c4cnn(C)c4)nc3c2c1] |
| | (3R)-3-{[9-methyl-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.12 min; MS (ESIneg): m/z = 389 [M − H]⁻ |
| | ¹H-NMR (400 MHz, DMSO-d₆): δ [ppm] = 1.25-1.37 (m, 1H), 1.46-1.59 (m, |
| | 1H), 1.85 (ddd, 2H), 1.97-2.06 (m, 1H), 2.25-2.35 (m, 1H), 3.10-3.20 (m, |
| | 1H), 3.95 (s, 3H), 4.80 (dd, 1H), 7.53 (d, 1H), 7.54-7.57 (m, 2H), 8.03-8.07 |
| | (m, 2H), 8.20 (dd, 1H), 8.47 (s, 1H). |
| Example 159 | 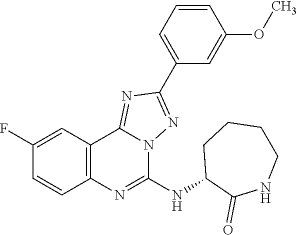 [SMILES: COc1cccc(-c2nc3c4cc(F)ccc4nc(N[C@@H]4CCCCNC4=O)n3n2)c1] |
| | (3R)-3-{[9-fluoro-2-(3-methoxyphenyl)[1,2,4]triazolo[1,5-c]quinazolin-5- |
| | yl]amino}azepan-2-one |
| | LC-MS (Method 2): Rₜ = 1.37 min; MS (ESIpos): m/z = 421 [M + H]⁺ |
| | ¹H-NMR (400 MHz, DMSO-d₆): δ [ppm] = 1.25-1.39 (m, 1H), 1.50-1.65 (m, |
| | 1H), 1.80-1.94 (m, 2H), 1.97-2.06 (m, 1H), 2.26-2.33 (m, 1H), 3.11-3.22 |
| | (m, 1H), 3.35-3.41 (m, 1H), 3.89 (s, 3H), 4.82 (dd, 1H), 7.15 (ddd, 1H), 7.52 |
| | (t, 1H), 7.59-7.66 (m, 1H), 7.69-7.77 (m, 2H), 7.79 (dd, 1H), 7.88 (dt, 1H), |
| | 8.02 (dd, 1H), 8.21 (dd, 1H). |
| Example 160 | 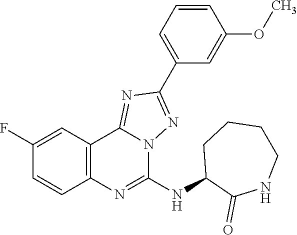 [SMILES: COc1cccc(-c2nc3c4cc(F)ccc4nc(N[C@H]4CCCCNC4=O)n3n2)c1] |
| | (3S)-3-{[9-fluoro-2-(3-methoxyphenyl)[1,2,4]triazolo[1,5-c]quinazolin-5- |
| | yl]amino}azepan-2-one |
| | LC-MS (Method 2): Rₜ = 1.37 min; MS (ESIpos): m/z = 421 [M + H]⁺ |
| Example 161 | 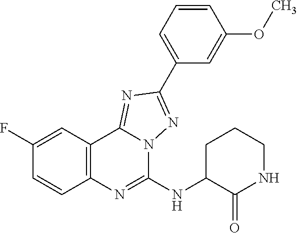 [SMILES: COc1cccc(-c2nc3c4cc(F)ccc4nc(NC4CCCNC4=O)n3n2)c1] |
| | 3-{[9-fluoro-2-(3-methoxyphenyl)[1,2,4]triazolo[1,5-c]quinazolin-5- |
| | yl]amino}piperidin-2-one |
| | LC-MS (method 2): Rₜ = 1.24 min; MS (ESIpos): m/z = 407 [M + H]⁺ |
| | ¹H-NMR (400 MHz, DMSO-d₆): δ [ppm] = 1.84-1.97 (m, 2H), 2.04-2.17 (m, |
| | 1H), 2.23-2.31 (m, 1H), 3.20-3.30 (m, 2H), 3.89 (s, 3H), 4.69 (dt, 1H), 7.15 |
| | (ddd, 1H), 7.52 (t, 1H), 7.57-7.64 (m, 1H), 7.64-7.71 (m, 1H), 7.79-7.85 |
| | (m, 2H), 7.90 (dt, 1H), 8.01 (dd, 1H), 8.22 (d, 1H). |
| Example 162 | 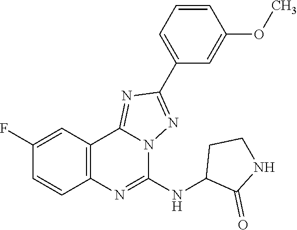 [SMILES: COc1cccc(-c2nc3c4cc(F)ccc4nc(NC4CCNC4=O)n3n2)c1] |
| | 3-{[9-fluoro-2-(3-methoxyphenyl)[1,2,4]triazolo[1,5-c]quinazolin-5- |
| | yl]amino}pyrrolidin-2-one |
| | LC-MS (Method 2): Rₜ = 1.17 min; MS (ESIpos): m/z = 393 [M + H]⁺ |
| | ¹H-NMR (400 MHz, DMSO-d₆): δ [ppm] = 2.24-2.38 (m, 1H), 2.53-2.57 (m, |
| | 1H), 3.30-3.33 (m, 1H), 3.34-3.37 (m, 1H), 3.89 (s, 3H), 4.88-5.00 (m, |
| | 1H), 7.11-7.18 (m, 1H), 7.52 (t, 1H), 7.59-7.71 (m, 2H), 7.84 (dd, 1H), |
| | 7.90 (d, 1H), 7.98-8.04 (m, 2H), 8.34 (d, 1H). |
| Example 163 | 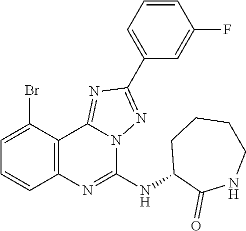 [SMILES: O=C1NCCCC[C@H]1Nc1nc2cccc(Br)c2c2nc(-c3cccc(F)c3)nn12] |
| | (3R)-3-{[10-bromo-2-(3-fluorophenyl)[1,2,4]triazolo[1,5-c]quinazolin-5- |
| | yl]amino}azepan-2-one |
| | LC-MS (Method 2): Rₜ = 1.54 min; MS (ESIpos): m/z = 469 [M + H]⁺ |
| | ¹H-NMR (400 MHz, DMSO-d₆): δ [ppm] = 1.26-1.39 (m, 1H), 1.52-1.64 (m, |
| | 1H), 1.80-1.94 (m, 2H), 1.97-2.07 (m, 1H), 2.27-2.35 (m, 1H), 3.12-3.21 |
| | (m, 1H), 3.39 (br d, 1H), 4.84 (br dd, 1H), 7.41-7.47 (m, 1H), 7.57-7.63 (m, |
| | 1H), 7.64-7.71 (m, 2H), 7.73 (dd, 1H), 7.89 (d, 1H), 7.99-8.04 (m, 1H), |
| | 8.15 (dt, 1H), 8.23 (dd, 1H). |
| Example 164 | 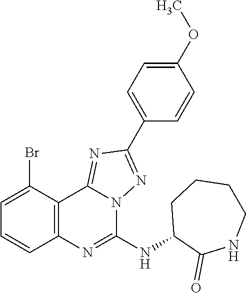 [SMILES: COc1ccc(-c2nc3c4c(Br)cccc4nc(N[C@@H]4CCCCNC4=O)n3n2)cc1] |
| | (3R)-3-{[10-bromo-2-(4-methoxyphenyl[1,2,4]triazolo[1,5-c]quinazolin- |
| | 5-yl]amino}azepan-2-one |
| | LC-MS (Method 2): Rₜ = 1.50 min; MS (ESIpos): m/z = 481 [M + H]⁺ |
| | ¹H-NMR (400 MHz, DMSO-d₆): δ [ppm] = 1.25-1.40 (m, 1H), 1.50-1.64 (m, |
| | 1H), 1.80-1.94 (m, 2H), 1.97-2.07 (m, 1H), 2.26-2.35 (m, 1H), 3.11-3.22 |
| | (m, 1H), 3.34-3.41 (m, 1H), 3.86 (s, 3H), 4.83 (br dd, 1H), 7.12-7.19 (m, |
| | 2H), 7.54-7.61 (m, 1H), 7.63-7.67 (m, 1H), 7.71 (dd, 1H), 7.82 (d, 1H), |
| | 8.19-8.27 (m, 3H). |
| Example 165 | 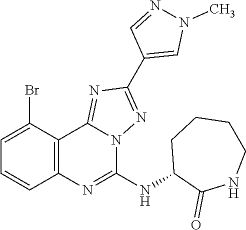 [SMILES: Cn1cc(-c2nc3c4c(Br)cccc4nc(N[C@@H]4CCCCNC4=O)n3n2)cn1] |
| | (3R)-3-{[10-bromo-2-(1-methyl-1H-pyrazol-4-yl)[1,2,4]triazolo[1,5- |
| | c]quinazolin-5-yl]amino}azepan-2-one |
| | LC-MS (Method 2): Rt = 1.21 min; MS (ESIpos): m/z = 455 [M + H]⁺ |
| | ¹H-NMR (400 MHz, DMSO-d₆): δ [ppm] = 1.24-1.38 (m, 1H), 1.47-1.61 (m, |
| | 1H), 1.79-1.94 (m, 2H), 1.97-2.07 (m, 1H), 2.25-2.35 (m, 1H), 3.11-3.20 |
| | (m, 1H), 3.29-3.40 (m, 1H), 3.96 (s, 3H), 4.82 (dd, 1H), 7.54-7.60 (m, 1H), |
| | 7.62-7.74 (m, 3H), 8.05 (s, 1H), 8.23 (br dd, 1H), 8.47 (s, 1H). |
| Example 166 | 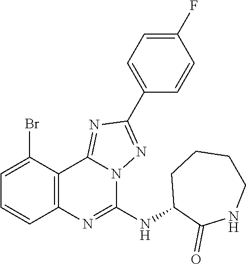 [SMILES: O=C1NCCCC[C@H]1Nc1nc2cccc(Br)c2c2nc(-c3ccc(F)cc3)nn12] |
| | (3R)-3-{[10-bromo-2-(4-fluorophenyl)[1,2,4]triazolo[1,5-c]quinazolin-5- |
| | yl]amino}azepan-2-one |
| | LC-MS (Method 2): Rₜ = 1.53 min; MS (ESIpos): m/z = 469 [M + H]⁺ |
| | ¹H-NMR (400 MHz, DMSO-d₆): δ [ppm] = 1.26-1.40 (m, 1H), 1.51-1.64 (m, |
| | 1H), 1.79-1.95 (m, 2H), 1.98-2.07 (m, 1H), 2.27-2.35 (m, 1H), 3.11-3.22 |
| | (m, 1H), 4.84 (dd, 1H), 7.42-7.50 (m, 1H), 7.57-7.63 (m, 1H), 7.65-7.69 |
| | (m, 1H), 7.72 (dd, 1H), 7.86 (d, 1H), 8.23 (dd, 1H), 8.31-8.39 (m, 2H). |
| Example 167 | 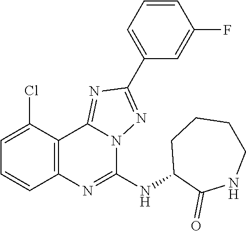 [SMILES: O=C1NCCCC[C@H]1Nc1nc2cccc(Cl)c2c2nc(-c3cccc(F)c3)nn12] |
| | (3R)-3-{[10-chloro-2-(3-fluorophenyl)[1,2,4]triazolo[1,5-c]quinazolin-5- |
| | yl]amino}azepan-2-one |
| | LC-MS (Method 2): Rₜ = 1.50 min; MS (ESIpos): m/z = 425 [M + H]⁺ |
| | ¹H-NMR (400 MHz, DMSO-d₆): δ [ppm] = 1.26-1.39 (m, 1H), 1.52-1.65 (m, |
| | 1H), 1.80-1.95 (m, 2H), 1.98-2.07 (m, 1H), 2.27-2.35 (m, 1H), 3.12-3.22 |
| | (m, 1H), 3.34-3.42 (m, 1H), 4.84 (br dd, 1H), 7.40-7.47 (m, 1H), 7.54 (dd, |
| | 1H), 7.60-7.71 (m, 3H), 7.90 (d, 1H), 7.97-8.02 (m, 1H), 8.15 (d, 1H), 8.23 |
| | (dd, 1H). |
| Example 168 | 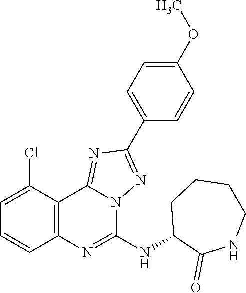 [SMILES: COc1ccc(-c2nc3c4c(Cl)cccc4nc(N[C@@H]4CCCCNC4=O)n3n2)cc1] |
| | (3R)-3-{[10-chloro-2-(4-methoxyphenyl)[1,2,4]triazolo[1,5-c]quinazolin- |
| | 5-yl]amino}azepan-2-one |
| | LC-MS (Method 2): Rₜ = 1.44 min; MS (ESIpos): m/z = 437 [M + H]⁺ |
| | ¹H-NMR (400 MHz, DMSO-d₆): δ [ppm] = 1.26-1.40 (m, 1H), 1.51-1.63 (m, |
| | 1H), 1.81-1.94 (m, 2H), 1.98-2.07 (m, 1H), 2.27-2.35 (m, 1H), 3.11-3.22 |
| | (m, 1H), 3.34-3.41 (m, 1H), 3.86 (s, 3H), 4.79-4.87 (m, 1H), 7.13-7.18 |
| | (m, 2H), 7.52 (dd, 1H), 7.59-7.69 (m, 2H), 7.84 (d, 1H), 8.20-8.26 (m, 3H). |
| Example 169 | 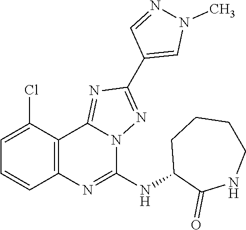 [SMILES: Cn1cc(-c2nc3c4c(Cl)cccc4nc(N[C@@H]4CCCCNC4=O)n3n2)cn1] |
| | (3R)-3-{[10-chloro-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.14 min; MS (ESIpos): m/z = 411 [M + H]⁺ |
| | ¹H-NMR (400 MHz, DMSO-d₆): δ [ppm] = 1.25-1.38 (m, 1H), 1.48-1.60 (m, |
| | 1H), 1.80-1.94 (m, 2H), 1.98-2.06 (m, 1H), 2.26-2.34 (m, 1H), 3.11-3.21 |
| | (m, 1H), 3.34-3.40 (m, 1H), 3.96 (s, 3H), 4.82 (dd, 1H), 7.51 (dd, 1H), 7.59- |
| | 7.69 (m, 2H), 7.73 (d, 1H), 8.06 (s, 1H), 8.23 (dd, 1H), 8.48 (s, 1H). |
| Example 170 | 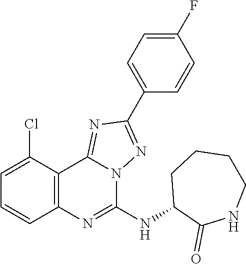 [SMILES: O=C1NCCCC[C@H]1Nc1nc2cccc(Cl)c2c2nc(-c3ccc(F)cc3)nn12] |
| | (3R)-3-{[10-chloro-2-(4-fluorophenyl)[1,2,4]triazolo[1,5-c]quinazolin-5- |
| | yl]amino}azepan-2-one |
| | LC-MS (Method 2): Rₜ = 1.49 min; MS (ESIpos): m/z = 425 [M + H]⁺ |
| | ¹H-NMR (400 MHz, DMSO-d₆): δ [ppm] = 1.25-1.39 (m, 1H), 1.51-1.64 (m, |
| | 1H), 1.80-1.95 (m, 2H), 1.98-2.07 (m, 1H), 2.27-2.36 (m, 1H), 3.11-3.22 |
| | (m, 1H), 4.84 (br dd, 1H), 7.45 (t, 2H), 7.54 (dd, 1H), 7.61-7.71 (m, 2H), |
| | 7.87 (d, 1H), 8.23 (br dd, 1H), 8.31-8.38 (m, 2H). |
| Example 171 | 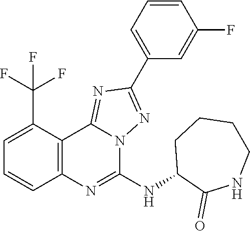 [SMILES: O=C1NCCCC[C@H]1Nc1nc2cccc(C(F)(F)F)c2c2nc(-c3cccc(F)c3)nn12] |
| | (3R)-3-{[2-(3-fluorophenyl)-10-(trifluoromethyl)[1,2,4]triazolo[1,5- |
| | c]quinazolin-5-yl]amino}azepan-2-one |
| | LC-MS (Method 2): Rₜ = 1.53 min; MS (ESIpos): m/z = 459 [M + H]⁺ |
| | ¹H-NMR (400 MHz, DMSO-d₆): δ [ppm] = 1.27-1.41 (m, 1H), 1.53-1.66 (m, |
| | 1H), 1.81-1.96 (m, 2H), 1.99-2.08 (m, 1H), 2.28-2.36 (m, 1H), 3.12-3.22 |
| | (m, 1H), 3.35-3.42 (m, 1H), 4.86 (dd, 1H), 7.41-7.48 (m, 1H), 7.69 (td, |
| | 1H), 7.84-7.90 (m, 2H), 7.92-8.01 (m, 3H), 8.13 (dt, 1H), 8.24 (dd, 1H). |
| Example 172 | 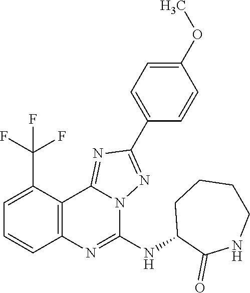 [SMILES: COc1ccc(-c2nc3c4c(C(F)(F)F)cccc4nc(N[C@@H]4CCCCNC4=O)n3n2)cc1] |
| | (3R)-3-{[2-(4-methoxyphenyl)-10-(trifluoromethyl)[1,2,4]triazolo[1,5- |
| | c]quinazolin-5-yl]amino}azepan-2-one |
| | LC-MS (Method 2): Rₜ = 1.50 min; MS (ESIpos): m/z = 471 [M + H]⁺ |
| | ¹H-NMR (400 MHz, DMSO-d₆): δ [ppm] = 1.26-1.40 (m, 1H), 1.52-1.65 (m, |
| | 1H), 1.80-1.95 (m, 2H), 1.98-2.08 (m, 1H), 2.28-2.36 (m, 1H), 3.12-3.22 |
| | (m, 1H), 3.34-3.42 (m, 1H), 3.86 (s, 3H), 4.85 (br dd, 1H), 7.14-7.20 (m, |
| | 2H), 7.82-7.97 (m, 4H), 8.19-8.27 (m, 3H). |
| Example 173 | 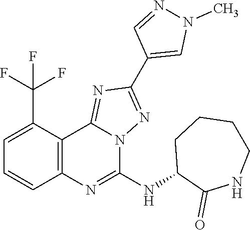 [SMILES: Cn1cc(-c2nc3c4c(C(F)(F)F)cccc4nc(N[C@@H]4CCCCNC4=O)n3n2)cn1] |
| | (3R)-3-{[2-(1-methyl-1H-pyrazol-4-yl)-10- |
| | (trifluoromethyl)[1,2,4]triazolo[1,5-c]quinazolin-5-yl]amino}azepan-2- |
| | one |
| | LC-MS (Method 2): Rₜ = 1.26 min; MS (ESIpos): m/z = 445 [M + H]⁺ |
| | ¹H-NMR (400 MHz, DMSO-d₆): δ [ppm] = 1.26-1.39 (m, 1H), 1.49-1.62 (m, |
| | 1H), 1.80-1.95 (m, 2H), 1.98-2.07 (m, 1H), 2.27-2.35 (m, 1H), 3.11-3.21 |
| | (m, 1H), 3.34-3.41 (m, 1H), 3.96 (s, 3H), 4.84 (dd, 1H), 7.79 (d, 1H), 7.82- |
| | 7.87 (m, 2H), 7.91-7.95 (m, 1H), 8.03 (d, 1H), 8.24 (dd, 1H), 8.44 (s, 1H). |
| Example 174 | 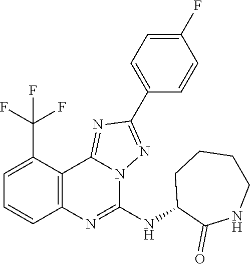 [SMILES: O=C1NCCCC[C@H]1Nc1nc2cccc(C(F)(F)F)c2c2nc(-c3ccc(F)cc3)nn12] |
| | (3R)-3-{[2-(4-fluorophenyl)-10-(trifluoromethyl)[1,2,4]triazolo[1,5- |
| | c]quinazolin-5-yl]amino}azepan-2-one |
| | LC-MS (Method 2): Rₜ = 1.53 min; MS (ESIneg): m/z = 457 [M − H]⁻ |
| | ¹H-NMR (400 MHz, DMSO-d₆): δ [ppm] = 1.26-1.40 (m, 1H), 1.52-1.65 (m, |
| | 1H), 1.81-1.96 (m, 2H), 1.99-2.07 (m, 1H), 2.29-2.37 (m, 1H), 3.12-3.23 |
| | (m, 1H), 3.35-3.42 (m, 1H), 4.86 (dd, 1H), 7.42-7.50 (m, 2H), 7.83-7.90 |
| | (m, 2H), 7.91-7.98 (m, 2H), 8.24 (dd, 1H), 8.29-8.36 (m, 2H). |
| Example 175 | 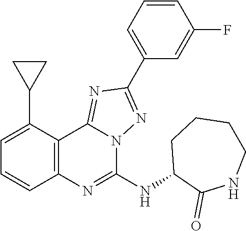 [SMILES: O=C1NCCCC[C@H]1Nc1nc2cccc(C3CC3)c2c2nc(-c3cccc(F)c3)nn12] |
| | (3R)-3-{[10-cyclopropyl-2-(3-fluorophenyl)[1,2,4]triazolo[1,5- |
| | c]quinazolin-5-yl]amino}azepan-2-one |
| | LC-MS (Method 2): Rₜ = 1.60 min; MS (ESIpos): m/z = 431 [M + H]⁺ |
| | ¹H-NMR (400 MHz, DMSO-d₆): δ [ppm] = 0.83-0.89 (m, 2H), 1.19-1.26 (m, |
| | 2H), 1.26-1.39 (m, 1H), 1.51-1.64 (m, 1H), 1.79-1.95 (m, 2H), 1.98-2.07 |
| | (m, 1H), 2.27-2.36 (m, 1H), 3.11-3.22 (m, 1H), 3.35-3.42 (m, 1H), 3.80 |
| | (tt, 1H), 4.83 (br dd, 1H), 6.98 (d, 1H), 7.39-7.49 (m, 2H), 7.56-7.62 (m, |
| | 1H), 7.66 (td, 1H), 7.75 (d, 1H), 7.98-8.04 (m, 1H), 8.11-8.17 (m, 1H), |
| | 8.21 (br dd, 1H). |
| Example 176 | 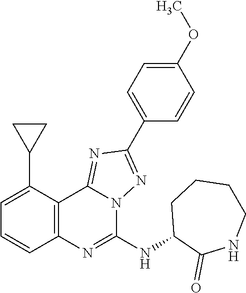 [SMILES: COc1ccc(-c2nc3c4c(C5CC5)cccc4nc(N[C@@H]4CCCCNC4=O)n3n2)cc1] |
| | (3R)-3-{[10-cyclopropyl-2-(4-methoxyphenyl)[1,2,4]triazolo[1,5- |
| | c]quinazolin-5-yl]amino}azepan-2-one |
| | LC-MS (Method 2): Rₜ = 1.55 min; MS (ESIpos): m/z = 443 [M + H]⁺ |
| | ¹H-NMR (400 MHz, DMSO-d₆): δ [ppm] = 0.82-0.89 (m, 2H), 1.19-1.25 (m, |
| | 2H), 1.26-1.39 (m, 1H), 1.50-1.62 (m, 1H), 1.80-1.94 (m, 2H), 1.97-2.07 |
| | (m, 1H), 2.28-2.37 (m, 1H), 3.11-3.21 (m, 1H), 3.34-3.41 (m, 1H), 3.79- |
| | 3.88 (m, 4H), 4.83 (br dd, 1H), 6.96 (d, 1H), 7.11-7.17 (m, 2H), 7.45 (dd, |
| | 1H), 7.54-7.61 (m, 1H), 7.70 (d, 1H), 8.18-8.25 (m, 3H). |
| Example 177 | 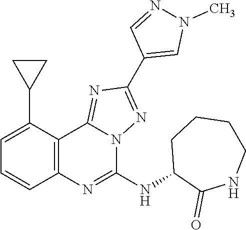 [SMILES: Cn1cc(-c2nc3c4c(C5CC5)cccc4nc(N[C@@H]4CCCCNC4=O)n3n2)cn1] |
| | (3R)-3-{[10-cyclopropyl-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.28 min; MS (ESIpos): m/z = 417 [M + H]⁺ |
| | ¹H-NMR (400 MHz, DMSO-d₆): δ [ppm] = 0.82-0.88 (m, 2H), 1.16-1.21 (m, |
| | 2H), 1.25-1.38 (m, 1H), 1.47-1.59 (m, 1H), 1.79-1.94 (m, 2H), 1.97-2.06 |
| | (m, 1H), 2.26-2.35 (m, 1H), 3.10-3.21 (m, 1H), 3.34-3.40 (m, 1H), 3.80 |
| | (tt, 1H), 3.95 (s, 3H), 4.81 (dd, 1H), 6.94 (d, 1H), 7.44 (dd, 1H), 7.54-7.61 |
| | (m, 2H), 8.06 (d, 1H), 8.20 (dd, 1H), 8.47 (s, 1H). |
| Example 178 | 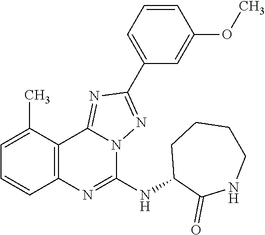 [SMILES: COc1cccc(-c2nc3c4c(C)cccc4nc(N[C@@H]4CCCCNC4=O)n3n2)c1] |
| | (3R)-3-{[2-(3-methoxyphenyl)-10-methyl[1,2,4]triazolo[1,5-c]quinazolin- |
| | 5-yl]amino}azepan-2-one |
| | LC-MS (Method 2): Rₜ = 0.92 min; MS (ESIpos): m/z = 417 [M + H]⁺ |
| | ¹H-NMR (400 MHz, DMSO-d₆): δ [ppm] = 1.26-1.39 (m, 1H), 1.51-1.64 (m, |
| | 1H), 1.81-1.95 (m, 2H), 1.98-2.07 (m, 1H), 2.27-2.36 (m, 1H), 3.03 (s, |
| | 3H), 3.11-3.21 (m, 1H), 3.34-3.41 (m, 1H), 3.85-3.91 (m, 3H), 4.84 (dd, |
| | 1H), 7.15 (ddd, 1H), 7.28 (d, 1H), 7.48-7.55 (m, 2H), 7.57-7.63 (m, 1H), |
| | 7.73 (d, 1H), 7.80 (dd, 1H), 7.89 (dt, 1H), 8.20 (dd, 1H). |
| Example 179 | 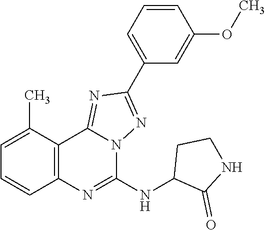 [SMILES: COc1cccc(-c2nc3c4c(C)cccc4nc(NC4CCNC4=O)n3n2)c1] |
| | 3-{[2-(3-methoxyphenyl)-10-methyl[1,2,4]triazolo[1,5-c]quinazolin-5- |
| | yl]amino}pyrrolidin-2-one |
| | LC-MS (Method 2): Rₜ = 0.90 min; MS (ESIpos): m/z = 389 [M + H]⁺ |
| | ¹H-NMR (400 MHz, DMSO-d₆): δ [ppm] = 2.25-2.38 (m, 1H), 3.03 (s, 3H), |
| | 3.35 (br d, 1H), 3.89 (s, 3H), 4.95 (dt, 1H), 7.15 (ddd, 1H), 7.24-7.29 (m, |
| | 1H), 7.46 (d, 1H), 7.53 (t, 1H), 7.59 (dd, 1H), 7.85 (dd, 1H), 7.92 (dt, 1H), |
| | 8.00 (s, 1H), 8.26 (d, 1H). |
| Example 180 | 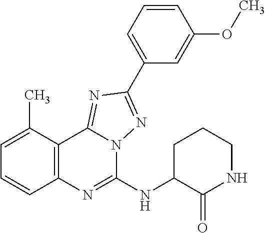 [SMILES: COc1cccc(-c2nc3c4c(C)cccc4nc(NC4CCCNC4=O)n3n2)c1] |
| | 3-{[2-(3-methoxyphenyl)-10-methyl[1,2,4]triazolo[1,5-c]quinazolin-5- |
| | yl]amino}piperidin-2-one |
| | LC-MS (Method 2): Rₜ = 0.83 min; MS (ESIpos): m/z = 403 [M + H]⁺ |
| | ¹H-NMR (400 MHz, DMSO-d₆): δ [ppm] = 1.85-1.98 (m, 2H), 2.04-2.16 (m, |
| | 1H), 2.25-2.34 (m, 1H), 3.03 (s, 3H), 3.21-3.29 (m, 2H), 3.89 (s, 3H), 4.66- |
| | 4.74 (m, 1H), 7.14 (ddd, 1H), 7.23-7.28 (m, 1H), 7.47 (d, 1H), 7.52 (t, 1H), |
| | 7.56-7.62 (m, 1H), 7.80 (br s, 1H), 7.84 (dd, 1H), 7.91 (dt, 1H), 8.14 (d, |
| | 1H). |
| Example 181 | 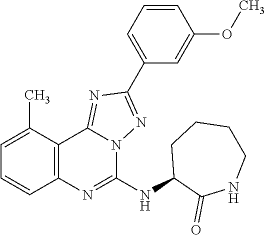 [SMILES: COc1cccc(-c2nc3c4c(C)cccc4nc(N[C@H]4CCCCNC4=O)n3n2)c1] |
| | (3S)-3-{[2-(3-methoxyphenyl)-10-methyl[1,2,4]triazolo[1,5-c]quinazolin- |
| | 5-yl]amino}azepan-2-one |
| | LC-MS (Method 2): Rₜ = 0.93 min; MS (ESIpos): m/z = 417 [M + H]⁺ |
| Example 182 | 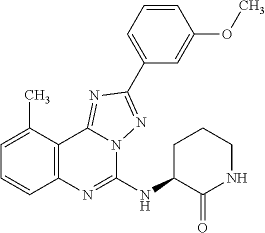 [SMILES: COc1cccc(-c2nc3c4c(C)cccc4nc(N[C@H]4CCCNC4=O)n3n2)c1] |
| | (3S)-3-{[2-(3-methoxyphenyl)-10-methyl[1,2,4]triazolo[1,5-c]quinazolin- |
| | 5-yl]amino}piperidin-2-one |
| | LC-MS (Method 2): Rₜ = 1.37 min; MS (ESIpos): m/z = 403 [M + H]⁺ |
| Example 183 | 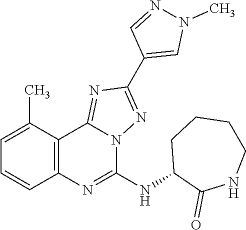 [SMILES: Cc1cccc2nc(N[C@@H]3CCCCNC3=O)n3nc(-c4cnn(C)c4)nc3c12] |
| | (3R)-3-{[10-methyl-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.22 min; MS (ESIpos): m/z = 391 [M + H]⁺ |
| | ¹H-NMR (400 MHz, DMSO-d₆): δ [ppm] = 1.25-1.38 (m, 1H), 1.46-1.60 (m, |
| | 1H), 1.79-1.94 (m, 2H), 1.95-2.07 (m, 1H), 2.24-2.35 (m, 1H), 2.99 (s, |
| | 3H), 3.10-3.21 (m, 1H), 3.34-3.41 (m, 1H), 3.95 (s, 3H), 4.81 (dd, 1H), |
| | 7.25 (d, 1H), 7.47-7.52 (m, 1H), 7.55-7.62 (m, 2H), 8.06 (d, 1H), 8.21 (dd, |
| | 1H), 8.48 (s, 1H). |
| Example 184 | 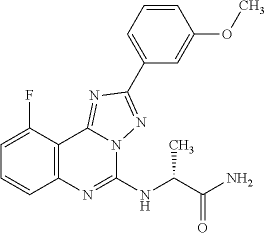 [SMILES: COc1cccc(-c2nc3c4c(F)cccc4nc(N[C@H](C)C(N)=O)n3n2)c1] |
| | N²-[10-fluoro-2-(3-methoxyphenyl)[1,2,4]triazolo[1,5-c]quinazolin-5-yl]- |
| | D-alaninamide |
| | LC-MS (Method 2): Rₜ = 1.06 min; MS (ESIpos): m/z = 381 [M + H]⁺ |
| | ¹H-NMR (400 MHz, DMSO-d₆): δ [ppm] = 1.55 (d, 3H), 3.89 (s, 3H), 4.76 |
| | (quin, 1H), 7.16 (br d, 1H), 7.21-7.32 (m, 2H), 7.42-7.49 (m, 1H), 7.52 (s, |
| | 1H), 7.66 (s, 2H), 7.82 (s, 1H), 7.90 (d, 1H), 7.99 (d, 1H). |
| Example 185 | 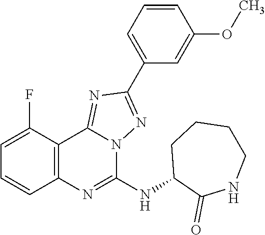 [SMILES: COc1cccc(-c2nc3c4c(F)cccc4nc(N[C@@H]4CCCCNC4=O)n3n2)c1] |
| | (3R)-3-{[10-fluoro-2-(3-methoxyphenyl[1,2,4]triazolo[1,5-c]quinazolin-5- |
| | yl]amino}azepan-2-one |
| | LC-MS (Method 2): Rₜ = 1.30 min; MS (ESIpos): m/z = 421 [M + H]⁺ |
| | ¹H-NMR (400 MHz, DMSO-d₆): δ [ppm] = 1.25-1.40 (m, 1H), 1.51-1.66 (m, |
| | 1H), 1.79-1.94 (m, 2H), 1.96-2.07 (m, 1H), 2.30-2.35 (m, 1H), 3.10-3.24 |
| | (m, 1H), 3.34-3.42 (m, 1H), 3.89 (s, 3H), 4.84 (br dd, 1H), 7.12-7.18 (m, |
| | 1H), 7.27 (dd, 1H), 7.47-7.55 (m, 2H), 7.72 (td, 1H), 7.77 (dd, 1H), 7.85- |
| | 7.92 (m, 2H), 8.22 (dd, 1H). |

### Example 186

### 3-{[9-fluoro-2-(3-methoxyphenyl)[1,2,4]triazolo[1,5-c]quinazolin-5-yl]amino}pyrrolidin-2-one, Enantiomer 1

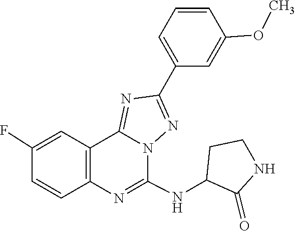 [SMILES: COc1cccc(-c2nc3c4cc(F)ccc4nc(NC4CCNC4=O)n3n2)c1]

Chiral HPLC separation of example 162 was performed (Instrument: Labomatic HD5000, Labocord-5000; Gilson GX-241, Labcol Vario 4000, column: Chiralpak IG 5μ 250×30 mm; Eluent A: acetonitrile+0.1 vol-% diethylamine (99%); Eluent B: ethanol; isocratic: 90% A+10% B; flow rate 50.0 mL/min; UV 254 nm).

Retention time of enantiomer 1: 2.56 min; [α]²⁰_{D}: +3° (c=1) in DMSO.

Instrument: Agilent HPLC 1260; Column: Chiralpak IG 3μ 100×4.6 mm; Eluent A: acetonitrile+0.1 vol-% diethylamine (99%); Eluent B: ethanol; isocratic: 90% A+10% B; flow rate 1.4 mL/min; temperature: 25° C.; DAD 254 nm.

### Example 187

### 3-{[9-fluoro-2-(3-methoxyphenyl)[1,2,4]triazolo[1,5-c]quinazolin-5-yl]amino}pyrrolidin-2-one, Enantiomer 2

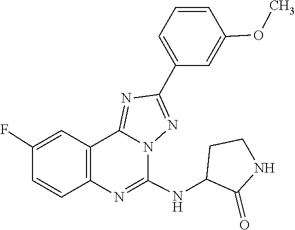 [SMILES: COc1cccc(-c2nc3c4cc(F)ccc4nc(NC4CCNC4=O)n3n2)c1]

The title compound was prepared as described for example 1.

Retention time of enantiomer 2: 4.41 min; [α]²⁰_{D}: −3° (c=1) in DMSO.

### Example 188

### 3-{[9-fluoro-2-(3-methoxyphenyl)[1,2,4]triazolo[1,5-c]quinazolin-5-yl]amino}piperidin-2-one, Enantiomer 1

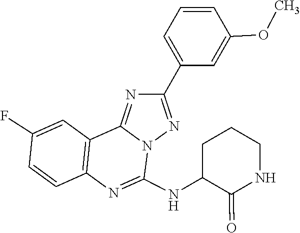 [SMILES: COc1cccc(-c2nc3c4cc(F)ccc4nc(NC4CCCNC4=O)n3n2)c1]

Chiral HPLC separation of example 161 was performed (Instrument: Labomatic HD5000, Labocord-5000; Gilson GX-241, Labcol Vario 4000, Column: Chiralpak IG 5μ 250×30 mm; Eluent: acetonitrile+0.1 vol-% diethylamine (99%)/ethanol 90:10%; flow rate 40.0 mL/min; UV 254 nm).

Retention time of enantiomer 1: 2.20 min; [α]²⁰_{D}: −29° (c=1) in DMSO.

Instrument: Agilent HPLC 1260; Column: Chiralpak IG 3μ 100×4.6 mm; Eluent: acetonitrile+0.1 vol-% diethylamine (99%)/ethanol 90:10; flow rate 1.4 mL/min; temperature: 25° C.; DAD 254 nm.

### Example 189

### 3-{[9-fluoro-2-(3-methoxyphenyl)[1,2,4]triazolo[1,5-c]quinazolin-5-yl]amino}piperidin-2-one, Enantiomer 2

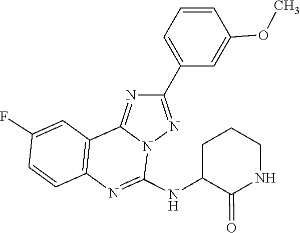 [SMILES: COc1cccc(-c2nc3c4cc(F)ccc4nc(NC4CCCNC4=O)n3n2)c1]

The title compound was prepared as described for example 188.

Retention time of enantiomer 2: 4.28 min; [α]²⁰_{D}: +31° (c=1) in DMSO.

### Example 190

### 3-{[2-(3-methoxyphenyl)-10-methyl[1,2,4]triazolo[1,5-c]quinazolin-5-yl]amino}pyrrolidin-2-one, Enantiomer 1

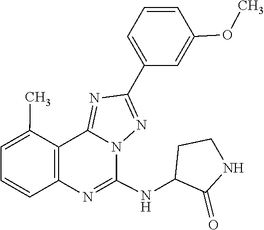 [SMILES: COc1cccc(-c2nc3c4c(C)cccc4nc(NC4CCNC4=O)n3n2)c1]

Chiral HPLC separation of example 179 was performed (Instrument: Labomatic HD5000, Labocord-5000; Gilson GX-241, Labcol Vario 4000, Column: YMC Cellulose SB 5μ 250×30 mm; Eluent: hexane+0.1 vol-% diethylamine (99%)/2-propanol 60:40; flow rate 50.0 mL/min; UV 254 nm).

Retention time of enantiomer 1: 2.70 min; [α]²⁰_{D}: +2° (c=1) in DMSO.

Instrument: Agilent HPLC 1260; Column: YMC Cellulose SB 3μ 100×4.6 mm; Eluent: hexane+0.1 vol-% diethylamine (99%)/2-propanol 60:40; flow rate 1.4 mL/min; temperature: 25° C.; DAD 254 nm.

### Example 191

### 3-{[2-(3-methoxyphenyl)-10-methyl[1,2,4]triazolo[1,5-c]quinazolin-5-yl]amino}pyrrolidin-2-one, Enantiomer 2

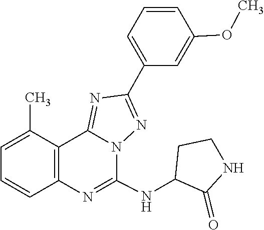 [SMILES: COc1cccc(-c2nc3c4c(C)cccc4nc(NC4CCNC4=O)n3n2)c1]

The title compound was prepared as described for example 190.

Retention time of enantiomer 1: 4.00 min.

### Example 192

### 6-{[2-(4-methoxyphenyl)-10-methyl[1,2,4]triazolo[1,5-c]quinazolin-5-yl]amino}-1,4-diazepan-5-one

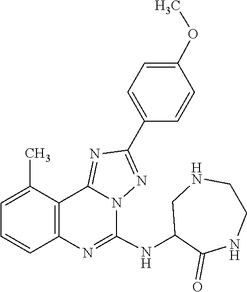 [SMILES: COc1ccc(-c2nc3c4c(C)cccc4nc(NC4CNCCNC4=O)n3n2)cc1]

Benzyl 6-{[2-(4-methoxyphenyl)-10-methyl[1,2,4]triazolo[1,5-c]quinazolin-5-yl]amino}-5-oxo-1,4-diazepane-1-carboxylate (25.0 mg, 45.3 μmol) was solubilized in DMF (2.0 mL). The reaction mixture was placed under an atmosphere of argon, Pd/C (4.82 mg,) in DMF (1 mL) was added. The reaction was place under an atmosphere of hydrogen and it was stirred for 2 h at rt. The mixture was filtered over Celite® and concentrated under reduced pressure. The crude mixture was purified without work up by preparative HPLC to give 7.50 mg (95% purity, 38% yield) of the title compound.

LC-MS (method 2): Rₜ=1.23 min; MS (ESIpos): m/z=418 [M+H]⁺

¹H-NMR (400 MHz, DMSO-d6): δ [ppm]=2.64-2.75 (m, 1H), 3.03 (s, 3H), 3.05-3.18 (m, 3H), 3.86 (s, 3H), 4.85-4.93 (m, 1H), 7.12-7.18 (m, 2H), 7.25-7.31 (m, 1H), 7.50-7.55 (m, 1H), 7.57-7.63 (m, 1H), 7.66 (d, 1H), 8.20-8.26 (m, 2H), 8.30 (br dd, 1H).

### Example 193

### (3R)-3-{[7-Chloro-2-(pyridin-4-yl)[1,2,4]triazolo[1,5-c]quinazolin-5-yl]amino}azepan-2-one

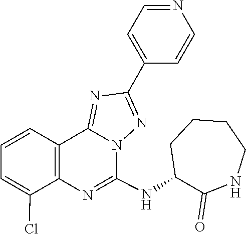 [SMILES: O=C1NCCCC[C@H]1Nc1nc2c(Cl)cccc2c2nc(-c3ccncc3)nn12]

5,7-Dichloro-2-(pyridin-4-yl)[1,2,4]triazolo[1,5-c]quinazoline (75.0 mg, 237 μmol), (3R)-3-aminoazepan-2-one (33.4 mg, 261 μmol) and N,N-diisopropylethylamine (120 μL, 710 μmol) were stirred in DMSO (1.6 mL) for 2 h at 60° C. The reaction was quenched with water and the suspension was filtered. The solid was washed with water and dried under reduced pressure at 60° C. The residue was purified by preparative HPLC followed by preparative TLC to give 2.80 mg (90% purity, 3% yield) of the title compound.

LC-MS (method 2): Rₜ=1.26 min; MS (ESIpos): m/z=408 [M+H]⁺

¹H-NMR (400 MHz, DMSO-d₆): δ [ppm]=1.36-1.46 (m, 1H), 1.50-1.62 (m, 1H), 1.82-1.97 (m, 2H), 2.02-2.10 (m, 1H), 2.38-2.46 (m, 1H), 3.14-3.24 (m, 1H), 4.85 (br dd, 1H), 7.45 (t, 1H), 7.91 (d, 1H), 7.94 (dd, 1H), 8.17-8.22 (m, 2H), 8.24-8.33 (m, 2H), 8.80-8.85 (m, 2H).

### Example 194

### (3R)-3-{[7-Chloro-2-(1-methyl-1H-pyrazol-4-yl)[1,2,4]triazolo[1,5-c]quinazolin-5-yl]amino}azepan-2-one

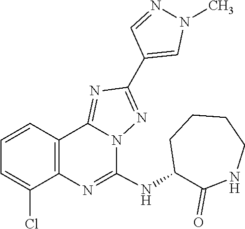 [SMILES: Cn1cc(-c2nc3c4cccc(Cl)c4nc(N[C@@H]4CCCCNC4=O)n3n2)cn1]

5,7-Dichloro-2-(1-methyl-1H-pyrazol-4-yl)[1,2,4]triazolo[1,5-c]quinazoline (75.0 mg, 235 μmol), (3R)-3-aminoazepan-2-one (33.1 mg, 258 μmol) and N,N-diisopropylethylamine (120 μL, 700 μmol) were stirred in DMSO (1.6 mL) for 2 h at 60° C. The reaction was quenched with water and the suspension was filtered. The solid was washed with water and dried under reduced pressure at 60° C. to give 88.9 mg (95% purity, 87% yield) of the title compound.

LC-MS (method 2): Rₜ=1.17 min; MS (ESIpos): m/z=411 [M+H]⁺

¹H-NMR (400 MHz, DMSO-d₆): δ [ppm]=1.26-1.39 (m, 1H), 1.47-1.59 (m, 1H), 1.82-1.96 (m, 2H), 2.00-2.09 (m, 1H), 2.41 (br d, 1H), 3.13-3.23 (m, 1H), 3.95 (s, 3H), 4.82 (dd, 1H), 7.40 (t, 1H), 7.71 (d, 1H), 7.89 (dd, 1H), 8.07 (d, 1H), 8.20 (dd, 1H), 8.24 (dd, 1H), 8.50 (s, 1H).

The following compounds were synthesised analogously to example 194:

| | Structure |
|---|---|
| | IUPAC-Name |
| | LC-MS (method): Retention time; Mass found |
| Example | ¹H-NMR |
| Example 195 | 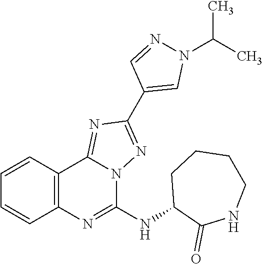 [SMILES: CC(C)n1cc(-c2nc3c4ccccc4nc(N[C@@H]4CCCCNC4=O)n3n2)cn1] |
| | (3R)-3-({2-[1-(propan-2-yl)-1H-pyrazol-4-yl][1,2,4]triazolo[1,5- |
| | c]quinazolin-5-yl]amino)azepan-2-one |
| | LC-MS (Method 2): Rₜ = 1.15 min; MS (ESIpos): m/z = 405 [M + H]⁺ |
| | ¹H-NMR (400 MHz, DMSO-d₆): δ [ppm] = 1.25-1.39 (m, 1H), 1.49 (d, 6H), |
| | 1.52-1.60 (m, 1H), 1.79-1.95 (m, 2H), 1.97-2.07 (m, 1H), 2.26-2.35 (m, |
| | 1H), 3.11-3.21 (m, 1H), 4.63 (sept, 1H), 4.79-4.86 (m, 1H), 7.44 (ddd, 1H), |
| | 7.60 (d, 1H), 7.64-7.68 (m, 1H), 7.70-7.76 (m, 1H), 8.09 (d, 1H), 8.20 (dd, |
| | 1H), 8.25 (dd, 1H), 8.52 (s, 1H). |
| Example 196 | 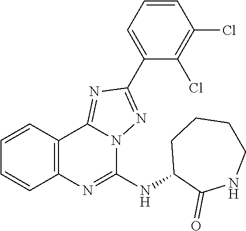 [SMILES: O=C1NCCCC[C@H]1Nc1nc2ccccc2c2nc(-c3cccc(Cl)c3Cl)nn12] |
| | (3R)-3-{[2-(2,3-dichlorophenyl)[1,2,4]triazolo[1,5-c]quinazolin-5- |
| | yl]amino}azepan-2-one |
| | LC-MS (Method 2): Rₜ = 1.43 min; MS (ESIpos): m/z = 441 [M + H]⁺ |
| | ¹H-NMR (400 MHz, DMSO-d₆): δ [ppm] = 1.25-1.40 (m, 1H), 1.49-1.61 (m, |
| | 1H), 1.80-1.96 (m, 2H), 1.98-2.08 (m, 1H), 2.30-2.38 (m, 1H), 3.11-3.21 |
| | (m, 1H), 3.36 (br d, 1H), 4.83 (dd, 1H), 7.47 (ddd, 1H), 7.58 (t, 1H), 7.68- |
| | 7.72 (m, 1H), 7.74-7.80 (m, 2H), 7.89 (dd, 1H), 7.98 (dd, 1H), 8.22 (dd, |
| | 1H), 8.29 (dd, 1H). |
| Example 197 | 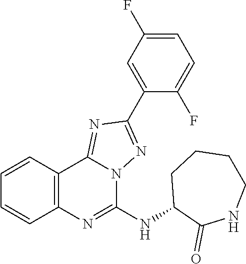 [SMILES: O=C1NCCCC[C@H]1Nc1nc2ccccc2c2nc(-c3cc(F)ccc3F)nn12] |
| | (3R)-3-{[2-(2,5-difluorophenyl)[1,2,4]triazolo[1,5-c]quinazolin-5- |
| | yl]amino}azepan-2-one |
| | LC-MS (Method 2): Rₜ = 1.36 min; MS (ESIpos): m/z = 409 [M + H]⁺ |
| | ¹H-NMR (400 MHz, DMSO-d₆): δ [ppm] = 1.26-1.39 (m, 1H), 1.50-1.62 (m, |
| | 1H), 1.80-1.93 (m, 2H), 1.97-2.09 (m, 1H), 2.25-2.40 (m, 1H), 3.12- |
| | 3.22 (m, 1H), 3.34-3.41 (m, 1H), 4.83 (dd, 1H), 7.44-7.58 (m, 3H), 7.67- |
| | 7.79 (m, 3H), 8.01 (ddd, 1H), 8.23 (dd, 1H), 8.31 (dd, 1H). |
| Example 198 | 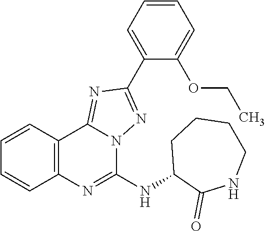 [SMILES: CCOc1ccccc1-c1nc2c3ccccc3nc(N[C@@H]3CCCCNC3=O)n2n1] |
| | (3R)-3-{[2-(2-ethoxyphenyl)[1,2,4]triazolo[1,5-c]quinazolin-5- |
| | yl]amino}azepan-2-one |
| | LC-MS (Method 2): Rₜ = 1.32 min; MS (ESIpos): m/z = 417 [M + H]⁺ |
| | ¹H-NMR (400 MHz, DMSO-d₆): δ [ppm] = 1.21-1.39 (m, 1H), 1.45 (t, 3H), |
| | 1.50-1.59 (m, 1H), 1.80-1.96 (m, 2H), 1.97-2.08 (m, 1H), 2.37 (br d, 1H), |
| | 3.11-3.21 (m, 1H), 3.35-3.41 (m, 1H), 4.13-4.22 (m, 2H), 4.81 (dd, 1H), |
| | 7.13 (td, 1H), 7.23 (d, 1H), 7.42-7.54 (m, 2H), 7.65-7.77 (m, 2H), 7.79 (d, |
| | 1H), 8.09 (dd, 1H), 8.22 (dd, 1H), 8.28 (dd, 1H). |
| Example 199 | 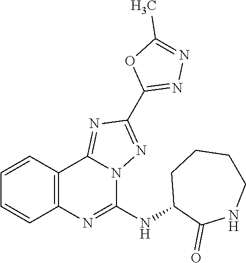 [SMILES: Cc1nnc(-c2nc3c4ccccc4nc(N[C@@H]4CCCCNC4=O)n3n2)o1] |
| | (3R)-3-{[2-(5-methyl-1,3,4-oxadiazol-2-yl)[1,2,4]triazolo[1,5-c]quinazolin- |
| | 5-yl]amino}azepan-2-one |
| | LC-MS (Method 2): Rₜ = 1.00 min; MS (ESIpos): m/z = 379 [M + H]⁺ |
| | ¹H-NMR (400 MHz, DMSO-d₆): δ [ppm] = 1.26-1.39 (m, 1H), 1.50-1.62 (m, |
| | 1H), 1.80-1.95 (m, 2H), 1.97-2.07 (m, 1H), 2.29-2.38 (m, 1H), 2.70 (s, |
| | 3H), 3.11-3.22 (m, 1H), 3.34-3.42 (m, 1H), 4.83 (br dd, 1H), 7.47-7.53 |
| | (m, 1H), 7.69-7.74 (m, 1H), 7.76-7.85 (m, 2H), 8.26 (dd, 1H), 8.33 (dd, |
| | 1H). |
| Example 200 | 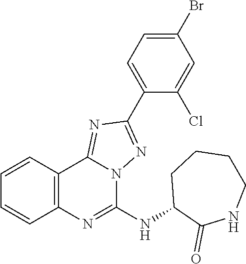 [SMILES: O=C1NCCCC[C@H]1Nc1nc2ccccc2c2nc(-c3ccc(Br)cc3Cl)nn12] |
| | (3R)-3-{[2-(4-bromo-2-chlorophenyl)[1,2,4]triazolo[1,5-c]quinazolin-5- |
| | yl]amino}azepan-2-one |
| | LC-MS (Method 2): Rₜ = 1.50 min; MS (ESIpos): m/z = 485 [M + H]⁺ |
| | ¹H-NMR (400 MHz, DMSO-d₆): δ [ppm] = 1.21-1.39 (m, 1H), 1.49-1.61 (m, |
| | 1H), 1.80-1.95 (m, 2H), 1.96-2.09 (m, 1H), 2.26-2.40 (m, 1H), 3.10- |
| | 3.21 (m, 1H), 3.34-3.41 (m, 1H), 4.83 (dd, 1H), 7.47 (ddd, 1H), 7.67-7.71 |
| | (m, 1H), 7.73-7.77 (m, 2H), 7.79 (dd, 1H), 8.00 (d, 1H), 8.05 (d, 1H), 8.21 |
| | (dd, 1H), 8.29 (dd, 1H). |
| Example 201 | 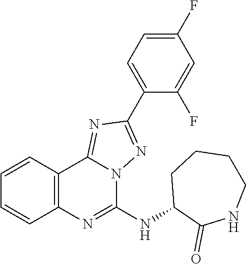 [SMILES: O=C1NCCCC[C@H]1Nc1nc2ccccc2c2nc(-c3ccc(F)cc3F)nn12] |
| | (3R)-3-{[2-(2,4-difluorophenyl)[1,2,4]triazolo[1,5-c]quinazolin-5- |
| | yl]amino}azepan-2-one |
| | LC-MS (Method 2): Rₜ = 1.33 min; MS (ESIpos): m/z = 409 [M + H]⁺ |
| | ¹H NMR (400 MHz, DMSO-d₆) δ ppm 8.28-8.38 (m, 2 H), 8.23 (dd, 1 H), |
| | 7.66-7.80 (m, 3 H), 7.42-7.60 (m, 2 H), 7.28-7.38 (m, 1 H), 4.82 (br dd, 1 |
| | H), 3.36-3.41 (m, 1 H), 3.08-3.24 (m, 1 H), 2.25-2.41 (m, 1 H), 1.96- |
| | 2.11 (m, 1 H), 1.76-1.95 (m, 2 H), 1.43-1.66 (m, 1 H), 1.19-1.42 (m, 1 H) |
| Example 202 | 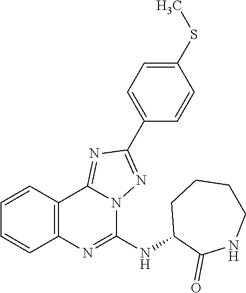 [SMILES: CSc1ccc(-c2nc3c4ccccc4nc(N[C@@H]4CCCCNC4=O)n3n2)cc1] |
| | (3R)-3-({2-[4-(methylsulfanyl)phenyl][1,2,4]triazolo[1,5-c]quinazolin-5- |
| | yl}amino)azepan-2-one |
| | LC-MS (Method 2): Rₜ = 1.43 min; MS (ESIpos): m/z = 419 [M + H]⁺ |
| | ¹H-NMR (400 MHz, DMSO-d₆): δ [ppm] = 1.26-1.39 (m, 1H), 1.51-1.63 (m, |
| | 1H), 1.81-1.95 (m, 2H), 1.99-2.07 (m, 1H), 2.29-2.36 (m, 1H), 2.56 (s, |
| | 3H), 3.12-3.21 (m, 1H), 3.34-3.41 (m, 1H), 4.83 (dd, 1H), 7.42-7.48 (m, |
| | 3H), 7.64-7.69 (m, 1H), 7.71-7.76 (m, 2H), 8.18-8.24 (m, 3H), 8.29 (dd, |
| | 1H). |
| Example 203 | 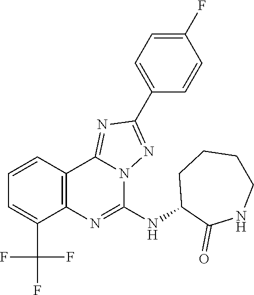 [SMILES: O=C1NCCCC[C@H]1Nc1nc2c(C(F)(F)F)cccc2c2nc(-c3ccc(F)cc3)nn12] |
| | (3R)-3-{[2-(4-fluorophenyl)-7-(trifluoromethyl)[1,2,4]triazolo[1,5- |
| | c]quinazolin-5-yl]amino}azepan-2-one |
| | LC-MS (Method 2): Rₜ = 1.51 min; MS (ESIpos): m/z = 459 [M + H]⁺ |
| | ¹H-NMR (400 MHz, DMSO-d₆): δ [ppm] = 1.26-1.39 (m, 1H), 1.48-1.61 (m, |
| | 1H), 1.78-1.92 (m, 2H), 1.99-2.08 (m, 1H), 2.41 (br d, 1H), 3.14-3.30 (m, |
| | 2H), 4.76 (dd, 1H), 7.40-7.48 (m, 2H), 7.56 (t, 1H), 7.91 (d, 1H), 8.09-8.14 |
| | (m, 1H), 8.24-8.30 (m, 1H), 8.30-8.37 (m, 2H), 8.56 (dd, 1H). |
| Example 204 | 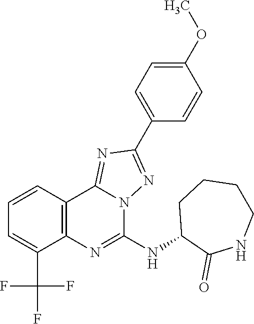 [SMILES: COc1ccc(-c2nc3c4cccc(C(F)(F)F)c4nc(N[C@@H]4CCCCNC4=O)n3n2)cc1] |
| | (3R)-3-{[2-(4-methoxyphenyl)-7-(trifluoromethyl)[1,2,4]triazolo[1,5- |
| | c]quinazolin-5-yl]amino}azepan-2-one |
| | LC-MS (Method 2): Rₜ = 1.47 min; MS (ESIpos): m/z = 471 [M + H]⁺ |
| | ¹H-NMR (400 MHz, DMSO-d₆): δ [ppm] = 1.26-1.39 (m, 1H), 1.48-1.61 (m, |
| | 1H), 1.78-1.92 (m, 2H), 1.98-2.08 (m, 1H,), 2.37-2.45 (m, 1H), 3.14-3.28 |
| | (m, 2H), 3.86 (s, 3H), 4.76 (br dd, 1H), 7.12-7.18 (m, 2H), 7.55 (t, 1H), 7.88 |
| | (d, 1H), 8.08-8.13 (m, 1H), 8.20-8.29 (m, 3H), 8.55 (dd, 1H). |
| Example 205 | 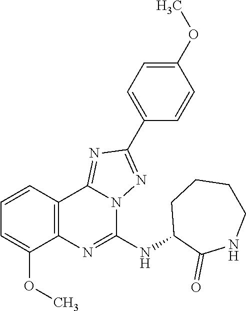 [SMILES: COc1ccc(-c2nc3c4cccc(OC)c4nc(N[C@@H]4CCCCNC4=O)n3n2)cc1] |
| | (3R)-3-{[7-methoxy-2-(4-methoxyphenyl)[1,2,4]triazolo[1,5-c]quinazolin- |
| | 5-yl]amino}azepan-2-one |
| | LC-MS (Method 2): Rₜ = 1.23 min; MS (ESIpos): m/z = 433 [M + H]⁺ |
| | ¹H-NMR (400 MHz, DMSO-d₆): δ [ppm] = 1.26-1.40 (m, 1H), 1.48- |
| | 1.60 (m, 1H), 1.80-1.93 (m, 2H), 1.96-2.09 (m, 1H), 2.33-2.42 (m, |
| | 1H), 3.11-3.23 (m, 1H), 3.28-3.32 (m, 1H), 3.86 (s, 3H), 3.96 (s, |
| | 3H), 4.76-4.88 (m, 1H), 7.10-7.18 (m, 2H), 7.28-7.33 (m, 1H), 7.33- |
| | 7.44 (m, 1H), 7.65 (d, 1H), 7.80-7.95 (m, 1H), 8.12-8.30 (m, 3H). |
| Example 206 | 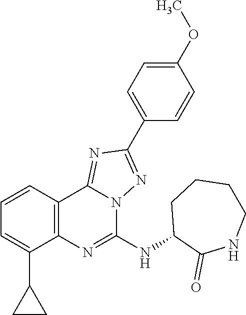 [SMILES: COc1ccc(-c2nc3c4cccc(C5CC5)c4nc(N[C@@H]4CCCCNC4=O)n3n2)cc1] |
| | (3R)-3-{[7-cyclopropyl-2-(4-methoxyphenyl)[1,2,4]triazolo[1,5- |
| | c]quinazolin-5-yl]amino}azepan-2-one |
| | LC-MS (Method 2): Rₜ = 1.49 min; MS (ESIpos): m/z = 443 [M + H]⁺ |
| | ¹H-NMR (400 MHz, DMSO-d₆): δ [ppm] = 0.79-0.95 (m, 2H), 1.05-1.12 (m, |
| | 2H), 1.26-1.39 (m, 1H), 1.49-1.62 (m, 1H), 1.79-1.92 (m, 2H), 2.04 (br |
| | dd, 1H), 2.43 (br d, 1H), 2.92 (tt, 1H), 3.12-3.21 (m, 1H), 3.34-3.40 (m, |
| | 1H), 3.86 (s, 3H), 4.85 (dd, 1H), 7.11-7.17 (m, 2H), 7.24-7.29 (m, 1H), |
| | 7.31-7.37 (m, 1H), 7.64 (d, 1H), 8.09 (dd, 1H), 8.19-8.25 (m, 3H). |
| Example 207 | 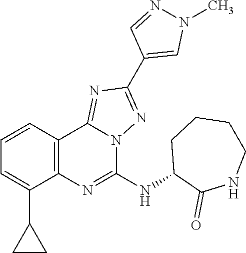 [SMILES: Cn1cc(-c2nc3c4cccc(C5CC5)c4nc(N[C@@H]4CCCCNC4=O)n3n2)cn1] |
| | (3R)-3-{[7-cyclopropyl-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.22 min; MS (ESIpos): m/z = 417 [M + H]⁺ |
| | ¹H-NMR (400 MHz, DMSO-d₆): δ [ppm] = 0.79-0.93 (m, 2H), 1.04-1.11 (m, |
| | 2H), 1.25-1.39 (m, 1H), 1.46-1.59 (m, 1H), 1.77-1.93 (m, 2H), 1.98-2.08 |
| | (m, 1H), 2.42 (br d, 1H), 2.86-2.96 (m, 1H), 3.11-3.21 (m, 1H), 3.29-3.40 |
| | (m, 1H), 3.95 (s, 3H), 4.84 (dd, 1H), 7.23-7.28 (m, 1H), 7.29-7.36 (m, 1H), |
| | 7.54 (d, 1H), 8.03 (dd, 1H), 8.06 (d, 1H), 8.22 (dd, 1H), 8.49 (s, 1H). |
| Example 208 | 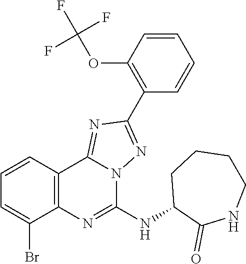 [SMILES: O=C1NCCCC[C@H]1Nc1nc2c(Br)cccc2c2nc(-c3ccccc3OC(F)(F)F)nn12] |
| | (3R)-3-({7-bromo-2-[2-(trifluoromethoxy)phenyl][1,2,4]triazolo[1,5- |
| | c]quinazolin-5-yl}amino)azepan-2-one |
| | LC-MS (Method 2): Rₜ = 1.57 min; MS (ESIpos): m/z = 535 [M + H]⁺ |
| | ¹H-NMR (400 MHz, DMSO-d₆): δ [ppm] = 1.23 (br s, 1H), 1.27-1.41 (m, 1H), |
| | 1.46-1.59 (m, 1H), 1.81-2.11 (m, 3H), 3.13-3.25 (m, 1H), 3.29 (br d, 1H), |
| | 4.82 (dd, 1H), 7.38 (t, 1H), 7.60-7.68 (m, 2H), 7.70-7.76 (m, 1H), 7.86 (d, |
| | 1H), 8.11 (dd, 1H), 8.23 (dd, 1H), 8.30 (dd, 1H), 8.40 (dd, 1H). |
| Example 209 | 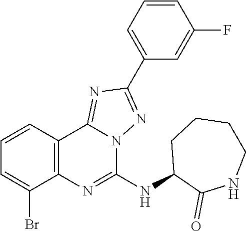 [SMILES: O=C1NCCCC[C@@H]1Nc1nc2c(Br)cccc2c2nc(-c3cccc(F)c3)nn12] |
| | (3S)-3-{[7-bromo-2-(3-fluorophenyl)[1,2,4]triazolo[1,5-c]quinazolin-5- |
| | yl]amino}azepan-2-one |
| | LC-MS (Method 2): Rₜ = 1.53 min; MS (ESIpos): m/z = 469 [M + H]⁺ |
| | ¹H-NMR (400 MHz, DMSO-d₆): δ [ppm] = 1.28-1.42 (m, 1H), 1.51-1.63 (m, |
| | 1H), 1.82-2.01 (m, 2H), 2.02-2.10 (m, 1H), 2.44 (br d, 1H), 3.15-3.24 (m, |
| | 1H), 4.85 (dd, 1H), 7.36 (t, 1H), 7.44 (td, 1H), 7.66 (td, 1H), 7.87 (d, 1H), |
| | 7.96-8.02 (m, 1H), 8.09 (dd, 1H), 8.13 (d, 1H), 8.25 (dd, 1H), 8.32 (dd, 1H). |
| Example 210 | 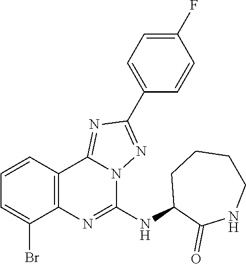 [SMILES: O=C1NCCCC[C@@H]1Nc1nc2c(Br)cccc2c2nc(-c3ccc(F)cc3)nn12] |
| | (3S)-3-{[7-bromo-2-(4-fluorophenyl)[1,2,4]triazolo[1,5-c]quinazolin-5- |
| | yl]amino}azepan-2-one |
| | LC-MS (Method 2): Rₜ = 1.52 min; MS (ESIpos): m/z = 469 [M + H]⁺ |
| | ¹H-NMR (400 MHz, DMSO-d₆): δ [ppm] = 1.27-1.40 (m, 1H), 1.49-1.61 (m, |
| | 1H), 1.83-2.01 (m, 2H), 2.02-2.10 (m, 1H), 2.43 (br s, 1H), 3.14-3.25 (m, |
| | 1H), 4.84 (dd, 1H), 7.36 (t, 1H), 7.39-7.47 (m, 2H), 7.84 (d, 1H), 8.09 (dd, |
| | 1H), 8.26 (dd, 1H), 8.29-8.36 (m, 3H). |
| Example 211 | 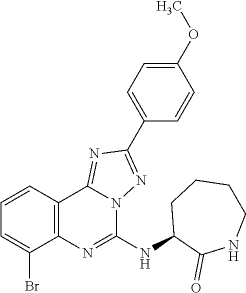 [SMILES: COc1ccc(-c2nc3c4cccc(Br)c4nc(N[C@H]4CCCCNC4=O)n3n2)cc1] |
| | (3S)-3-{[7-bromo-2-(4-methoxyphenyl)[1,2,4]triazolo[1,5-c]quinazolin-5- |
| | yl]amino}azepan-2-one |
| | LC-MS (Method 2): Rₜ = 1.48 min; MS (ESIpos): m/z = 481 [M + H]⁺ |
| | ¹H-NMR (400 MHz, DMSO-d₆): δ [ppm] = 1.29-1.41 (m, 1H), 1.49-1.61 (m, |
| | 1H), 1.82-2.00 (m, 2H), 2.02-2.10 (m, 1H), 2.41-2.47 (m, 1H), 3.14-3.24 |
| | (m, 1H), 3.86 (s, 3H), 4.84 (dd, 1H), 7.11-7.17 (m, 2H), 7.34 (t, 1H), 7.81 |
| | (d, 1H), 8.07 (dd, 1H), 8.18-8.27 (m, 3H), 8.30 (dd, 1H). |
| Example 212 | 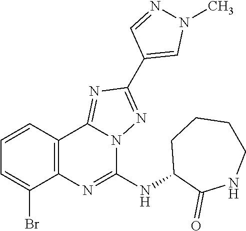 [SMILES: Cn1cc(-c2nc3c4cccc(Br)c4nc(N[C@@H]4CCCCNC4=O)n3n2)cn1] |
| | (3R)-3-{[7-bromo-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.18 min; MS (ESIpos): m/z = 455 [M + H]⁺ |
| | ¹H-NMR (400 MHz, DMSO-d₆): δ [ppm] = 1.26-1.42 (m, 1H), 1.44-1.59 (m, |
| | 1H), 1.82-2.00 (m, 2H), 2.01-2.09 (m, 1H), 2.39-2.47 (m, 1H), 3.14-3.23 |
| | (m, 1H), 3.25-3.29 (m, 1H), 3.95 (s, 3H), 4.82 (br dd, 1H), 7.33 (t, 1H), 7.70 |
| | (d, 1H), 8.04-8.11 (m, 2H), 8.21-8.29 (m, 2H), 8.50 (s, 1H). |
| Example 213 | 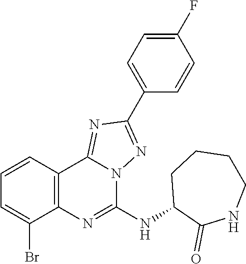 [SMILES: O=C1NCCCC[C@H]1Nc1nc2c(Br)cccc2c2nc(-c3ccc(F)cc3)nn12] |
| | (3R)-3-{[7-bromo-2-(4-fluorophenyl)[1,2,4]triazolo[1,5-c]quinazolin-5- |
| | yl]amino}azepan-2-one |
| | LC-MS (Method 2): Rₜ = 1.52 min; MS (ESIpos): m/z = 470 [M + H]⁺ |
| | ¹H-NMR (400 MHz, DMSO-d₆): δ [ppm] = 1.28-1.42 (m, 1H), 1.50-1.63 (m, |
| | 1H), 1.81-2.12 (m, 3H), 3.14-3.25 (m, 1H), 4.81-4.89 (m, 1H), 7.37 (t, |
| | 1H), 7.44 (td, 1H), 7.63-7.71 (m, 1H), 7.88 (d, 1H), 8.00 (br d, 1H), 8.07- |
| | 8.12 (m, 1H), 8.14 (d, 1H), 8.26 (br dd, 1H), 8.32 (d, 1H). |
| Example 214 | 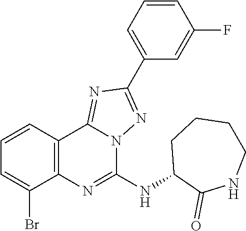 [SMILES: O=C1NCCCC[C@H]1Nc1nc2c(Br)cccc2c2nc(-c3cccc(F)c3)nn12] |
| | (3R)-3-{[7-bromo-2-(3-fluorophenyl)[1,2,4]triazolo[1,5-c]quinazolin-5- |
| | yl]amino}azepan-2-one |
| | LC-MS (Method 2): Rₜ = 1.50 min; MS (ESIpos): m/z = 470 [M + H]⁺ |
| | ¹H-NMR (400 MHz, DMSO-d₆): δ [ppm] = 1.28-1.41 (m, 1H), 1.49-1.62 (m, |
| | 1H), 1.82-2.10 (m, 3H), 2.41-2.47 (m, 1H), 3.14-3.24 (m, 1H), 3.27-3.38 |
| | (m, 1H), 4.84 (dd, 1H), 7.36 (t, 1H), 7.40-7.48 (m, 2H), 7.85 (d, 1H), 8.09 |
| | (dd, 1H), 8.26 (dd, 1H), 8.29-8.36 (m, 3H). |
| Example 215 | 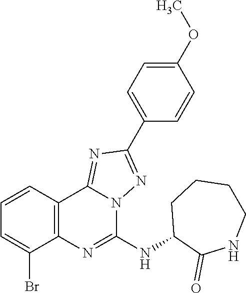 [SMILES: COc1ccc(-c2nc3c4cccc(Br)c4nc(N[C@@H]4CCCCNC4=O)n3n2)cc1] |
| | (3R)-3-{[7-bromo-2-(4-methoxyphenyl)[1,2,4]triazolo[1,5-c]quinazolin-5- |
| | yl]amino}azepan-2-one |
| | LC-MS (Method 2): Rₜ = 1.47 min; MS (ESIpos): m/z = 481 [M + H]⁺ |
| | ¹H-NMR (400 MHz, DMSO-d₆): δ [ppm] = 1.27-1.41 (m, 1H), 1.49-1.62 (m, |
| | 1H), 1.81-2.13 (m, 3H), 2.43 (br s, 1H), 3.13-3.25 (m, 1H), 3.86 (s, 3H), |
| | 4.84 (dd, 1H), 7.11-7.17 (m, 2H), 7.35 (t, 1H), 7.81 (d, 1H), 8.08 (dd, 1H), |
| | 8.19-8.27 (m, 3H), 8.30 (dd, 1H). |
| Example 216 | 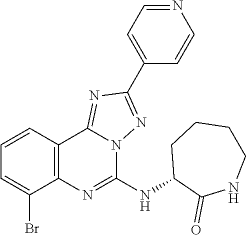 [SMILES: O=C1NCCCC[C@H]1Nc1nc2c(Br)cccc2c2nc(-c3ccncc3)nn12] |
| | (3R)-3-{[7-bromo-2-(pyridin-4-yl)[1,2,4]triazolo[1,5-c]quinazolin-5- |
| | yl]amino}azepan-2-one |
| | LC-MS (Method 2): Rₜ = 1.29 min; MS (ESIpos): m/z = 454 [M + H]⁺ |
| | ¹H-NMR (400 MHz, DMSO-d₆): δ [ppm] = 1.28-1.41 (m, 1H), 1.50-1.63 (m, |
| | 1H), 1.82-2.01 (m, 2H), 2.02-2.11 (m, 1H), 2.41-2.46 (m, 1H), 3.15-3.24 |
| | (m, 1H), 4.85 (dd, 1H), 7.38 (t, 1H), 7.90 (d, 1H), 8.11 (dd, 1H), 8.17-8.22 |
| | (m, 2H), 8.27 (dd, 1H), 8.34 (dd, 1H), 8.80-8.85 (m, 2H). |
| Example 217 | 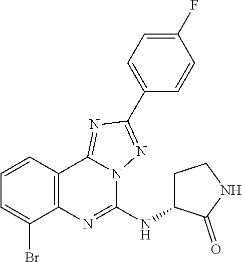 [SMILES: O=C1NCC[C@H]1Nc1nc2c(Br)cccc2c2nc(-c3ccc(F)cc3)nn12] |
| | (3R)-3-{[7-bromo-2-(4-fluorophenyl)[1,2,4]triazolo[1,5-c]quinazolin-5- |
| | yl]amino}pyrrolidin-2-one |
| | LC-MS (Method 2): Rₜ = 1.24 min; MS (ESIpos): m/z = 441 [M + H]⁺ |
| | ¹H-NMR (400 MHz, DMSO-d₆): δ [ppm] = 3.38-3.45 (m, 1H), 4.79-4.88 (m, |
| | 1H), 7.34 (t, 1H), 7.42-7.50 (m, 2H), 8.01-8.09 (m, 2H), 8.27-8.38 (m, |
| | 3H), 8.59 (d, 1H). |
| Example 218 | 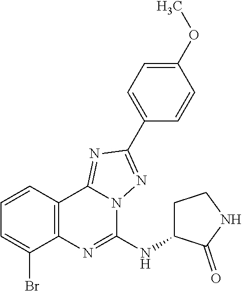 [SMILES: COc1ccc(-c2nc3c4cccc(Br)c4nc(N[C@@H]4CCNC4=O)n3n2)cc1] |
| | (3R)-3-{[7-bromo-2-(4-methoxyphenyl)[1,2,4]triazolo[1,5-c]quinazolin-5- |
| | yl]amino}pyrrolidin-2-one |
| | LC-MS (Method 2): Rₜ = 1.22 min; MS (ESIpos): m/z = 453 [M + H]⁺ |
| | ¹H-NMR (400 MHz, DMSO-d₆): δ [ppm] = 2.39-2.47 (m, 1H), 2.52-2.59 (m, |
| | 1H), 3.37-3.45 (m, 1H), 3.86 (s, 3H), 4.79-4.88 (m, 1H), 7.11-7.17 (m, |
| | 2H), 7.32 (t, 1H), 8.00-8.07 (m, 2H), 8.20-8.25 (m, 2H), 8.26-8.31 (m, |
| | 1H), 8.52 (d, 1H). |
| Example 219 | 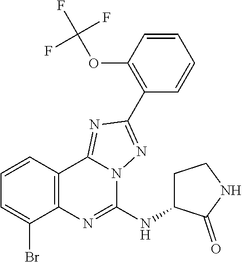 [SMILES: O=C1NCC[C@H]1Nc1nc2c(Br)cccc2c2nc(-c3ccccc3OC(F)(F)F)nn12] |
| | (3R)-3-({7-bromo-2-[2-(trifluoromethoxy)phenyl][1,2,4]triazolo[1,5- |
| | c]quinazolin-5-yl}amino)pyrrolidin-2-one |
| | LC-MS (Method 2): Rₜ = 1.33 min; MS (ESIpos): m/z = 507 [M + H]⁺ |
| | ¹H-NMR (400 MHz, DMSO-d₆): δ [ppm] = 2.37-2.47 (m, 1H), 2.56-2.66 (m, |
| | 1H), 3.34-3.45 (m, 2H), 4.73-4.83 (m, 1H), 7.36 (t, 1H), 7.59-7.69 (m, |
| | 2H), 7.70-7.76 (m, 1H), 8.06 (s, 1H), 8.09 (dd, 1H), 8.28 (dt, 2H), 8.38 (d, |
| | 1H). |
| Example 220 | 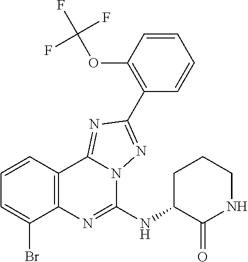 [SMILES: O=C1NCCC[C@H]1Nc1nc2c(Br)cccc2c2nc(-c3ccccc3OC(F)(F)F)nn12] |
| | (3R)-3-({7-bromo-2-[2-(trifluoromethoxy)phenyl][1,2,4]triazolo[1,5- |
| | c]quinazolin-5-yl}amino)piperidin-2-one |
| | LC-MS (Method 2): Rₜ = 1.41 min; MS (ESIpos): m/z = 521 [M + H]⁺ |
| | ¹H-NMR (400 MHz, DMSO-d₆): δ [ppm] = 1.79-2.01 (m, 2H), 2.25 (qd, 1H), |
| | 2.40-2.47 (m, 1H), 3.19-3.28 (m, 1H), 3.34-3.42 (m, 1H), 4.46-4.56 (m, |
| | 1H), 7.36 (t, 1H), 7.59-7.69 (m, 2H), 7.69-7.77 (m, 1H), 7.84 (br s, 1H), |
| | 8.09 (dd, 1H), 8.28 (ddd, 2H), 8.36 (d, 1H). |
| Example 221 | 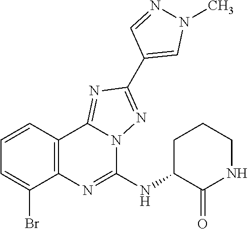 [SMILES: Cn1cc(-c2nc3c4cccc(Br)c4nc(N[C@@H]4CCCNC4=O)n3n2)cn1] |
| | (3R)-3-{[7-bromo-2-(1-methyl-1H-pyrazol-4-yl)[1,2,4]triazolo[1,5- |
| | c]quinazolin-5-yl]amino}piperidin-2-one |
| | LC-MS (Method 2): Rt = 1.01 min; MS (ESIpos): m/z = 441 [M + H]⁺ |
| | ¹H-NMR (400 MHz, DMSO-d₆): δ [ppm] = 1.77-2.03 (m, 2H), 2.25-2.32 (m, |
| | 1H), 3.16-3.27 (m, 1H), 3.96 (s, 3H), 4.41-4.57 (m, 1H), 7.31 (t, 1H), 7.79 |
| | (br s, 1H), 7.99-8.10 (m, 2H), 8.23 (dd, 1H), 8.36 (d, 1H), 8.43 (s, 1H). |
| Example 222 | 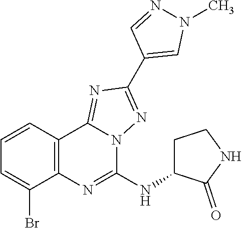 [SMILES: Cn1cc(-c2nc3c4cccc(Br)c4nc(N[C@@H]4CCNC4=O)n3n2)cn1] |
| | (3R)-3-{[7-bromo-2-(1-methyl-1H-pyrazol-4-yl)[1,2,4]triazolo[1,5- |
| | c]quinazolin-5-yl]amino}pyrrolidin-2-one |
| | LC-MS (Method 2): Rt = 0.93 min; MS (ESIpos): m/z = 427 [M + H]⁺ |
| | ¹H-NMR (400 MHz, DMSO-d₆): δ [ppm] = 1.49 (br s, 1H), 2.40-2.45 (m, 1H), |
| | 3.38-3.44 (m, 1H), 3.78 (s, 1H), 3.96 (s, 3H), 4.75-4.87 (m, 1H), 7.32 (t, |
| | 1H), 7.99-8.08 (m, 3H), 8.24 (br dd, 1H), 8.37-8.45 (m, 2H). |
| Example 223 | 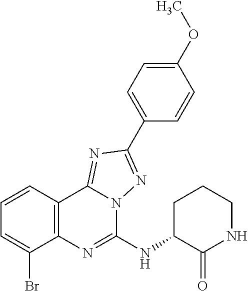 [SMILES: COc1ccc(-c2nc3c4cccc(Br)c4nc(N[C@@H]4CCCNC4=O)n3n2)cc1] |
| | (3R)-3-{[7-bromo-2-(4-methoxyphenyl)[1,2,4]triazolo[1,5-c]quinazolin-5- |
| | yl]amino}piperidin-2-one |
| | LC-MS (Method 2): Rₜ = 1.30 min; MS (ESIpos): m/z = 467 [M + H]⁺ |
| | ¹H-NMR (400 MHz, DMSO-d₆): δ [ppm] = 1.80-2.02 (m, 2H), 2.26-2.41 (m, |
| | 2H), 3.19-3.27 (m, 1H), 3.36-3.42 (m, 1H), 3.86 (s, 3H), 4.53 (dt, 1H), 7.12- |
| | 7.17 (m, 2H), 7.31 (t, 1H), 7.80 (br s, 1H), 8.05 (dd, 1H), 8.19-8.25 (m, |
| | 2H), 8.26-8.31 (m, 1H), 8.49 (d, 1H). |
| Example 224 | 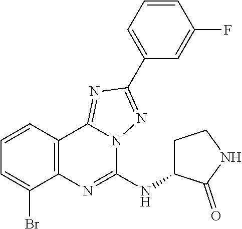 [SMILES: O=C1NCC[C@H]1Nc1nc2c(Br)cccc2c2nc(-c3cccc(F)c3)nn12] |
| | (3R)-3-{[7-bromo-2-(3-fluorophenyl)[1,2,4]triazolo[1,5-c]quinazolin-5- |
| | yl]amino}pyrrolidin-2-one |
| | LC-MS (Method 2): Rₜ = 1.26 min; MS (ESIpos): m/z = 441 [M + H]⁺ |
| | ¹H-NMR (400 MHz, DMSO-d₆): δ [ppm] = 3.37-3.45 (m, 1H), 4.80-4.90 (m, |
| | 1H), 7.34 (t, 1H), 7.40-7.47 (m, 1H), 7.67 (td, 1H), 7.98-8.09 (m, 3H), 8.14 |
| | (dt, 1H), 8.31 (dd, 1H), 8.64 (d, 1H). |
| Example 225 | 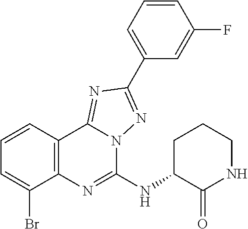 [SMILES: O=C1NCCC[C@H]1Nc1nc2c(Br)cccc2c2nc(-c3cccc(F)c3)nn12] |
| | (3R)-3-{[7-bromo-2-(3-fluorophenyl)[1,2,4]triazolo[1,5-c]quinazolin-5- |
| | yl]amino}piperidin-2-one |
| | LC-MS (Method 2): Rₜ = 1.34 min; MS (ESIpos): m/z = 455 [M + H]⁺ |
| | ¹H-NMR (400 MHz, DMSO-d₆): δ [ppm] = 1.80-2.02 (m, 2H), 2.24-2.41 (m, |
| | 2H), 3.20-3.28 (m, 1H), 3.36-3.43 (m, 1H), 4.55 (dt, 1H), 7.30-7.37 (m, |
| | 1H), 7.40-7.47 (m, 1H), 7.67 (td, 1H), 7.82 (br s, 1H), 8.00 (ddd, 1H), 8.07 |
| | (dd, 1H), 8.13 (dt, 1H), 8.30 (dd, 1H), 8.60 (d, 1H). |
| Example 226 | 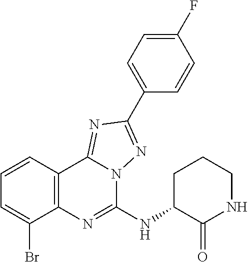 [SMILES: O=C1NCCC[C@H]1Nc1nc2c(Br)cccc2c2nc(-c3ccc(F)cc3)nn12] |
| | (3R)-3-{[7-bromo-2-(4-fluorophenyl)[1,2,4]triazolo[1,5-c]quinazolin-5- |
| | yl]amino}piperidin-2-one |
| | LC-MS (Method 2): Rₜ = 1.33 min; MS (ESIpos): m/z = 455 [M + H]⁺ |
| | ¹H-NMR (400 MHz, DMSO-d₆): δ [ppm] = 1.80-2.02 (m, 2H), 2.26-2.38 (m, |
| | 2H), 3.19-3.27 (m, 1H), 3.36-3.44 (m, 1H), 4.54 (dt, 1H), 7.29-7.35 (m, |
| | 1H), 7.40-7.49 (m, 2H), 7.82 (br s, 1H), 8.06 (dd, 1H), 8.26-8.36 (m, 3H), |
| | 8.56 (d, 1H). |
| Example 227 | 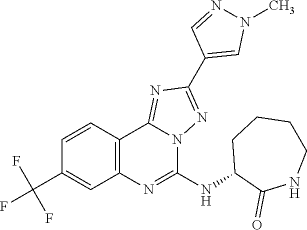 [SMILES: Cn1cc(-c2nc3c4ccc(C(F)(F)F)cc4nc(N[C@@H]4CCCCNC4=O)n3n2)cn1] |
| | (3R)-3-{[2-(1-methyl-1H-pyrazol-4-yl)-8- |
| | (trifluoromethyl)[1,2,4]triazolo[1,5-c]quinazolin-5-yl]amino}azepan-2- |
| | one |
| | LC-MS (Method 2): Rt = 1.22 min; MS (ESIpos): m/z = 445 [M + H]⁺ |
| | ¹H-NMR (400 MHz, DMSO-d₆): δ [ppm] = 1.32 (q, 1H), 1.50-1.62 (m, 1H), |
| | 1.81-1.93 (m, 2H), 1.97-2.06 (m, 1H), 2.26-2.35 (m, 1H), 3.12-3.22 (m, |
| | 1H), 3.96 (s, 3H), 4.84 (br dd, 1H), 7.69 (dd, 1H), 7.78 (d, 1H), 7.91 (s, 1H), |
| | 8.08 (s, 1H), 8.24 (dd, 1H), 8.42 (d, 1H), 8.51 (s, 1H). |
| Example 228 | 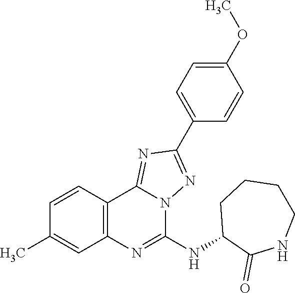 [SMILES: COc1ccc(-c2nc3c4ccc(C)cc4nc(N[C@@H]4CCCCNC4=O)n3n2)cc1] |
| | (3R)-3-{[2-(4-methoxyphenyl)-8-methyl[1,2,4]triazolo[1,5-c]quinazolin-5- |
| | yl]amino}azepan-2-one |
| | LC-MS (Method 2): Rₜ = 1.38 min; MS (ESIpos): m/z = 417 [M + H]⁺ |
| | ¹H-NMR (500 MHz, DMSO-d₆): δ [ppm] = 1.26-1.38 (m, 1H), 1.51-1.61 (m, |
| | 1H), 1.82-1.90 (m, 2H), 2.02 (br d, 1H), 2.31 (br d, 1H), 3.11-3.21 (m, 1H), |
| | 3.86 (s, 3H), 4.81 (br dd, 1H), 7.12-7.15 (m, 2H), 7.28 (dd, 1H), 7.49 (s, |
| | 1H), 7.67 (d, 1H), 8.17 (d, 1H), 8.19-8.23 (m, 3H). |
| Example 229 | 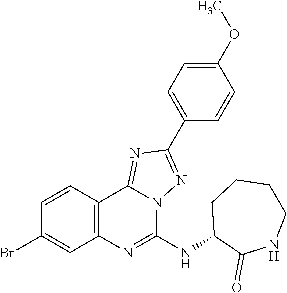 [SMILES: COc1ccc(-c2nc3c4ccc(Br)cc4nc(N[C@@H]4CCCCNC4=O)n3n2)cc1] |
| | (3R)-3-{[8-bromo-2-(4-methoxyphenyl)[1,2,4]triazolo[1,5-c]quinazolin-5- |
| | yl]amino}azepan-2-one |
| | LC-MS (Method 2): Rt = 1.50 min; MS (ESIpos): m/z = 481 [M + H]⁺ |
| | ¹H-NMR (500 MHz, DMSO-d₆): δ [ppm] = 1.27-1.38 (m, 1H), 1.52-1.62 (m, |
| | 1H), 1.81-1.94 (m, 2H), 1.98-2.05 (m, 1H), 2.30 (br d, 1H), 3.13-3.21 (m, |
| | 1H), 3.86 (s, 3H), 4.82 (dd, 1H), 7.13-7.16 (m, 2H), 7.59 (dd, 1H), 7.81- |
| | 7.84 (m, 2H), 8.19-8.24 (m, 4H). |
| Example 230 | 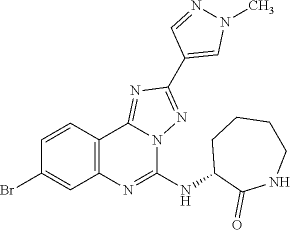 [SMILES: Cn1cc(-c2nc3c4ccc(Br)cc4nc(N[C@@H]4CCCCNC4=O)n3n2)cn1] |
| | (3R)-3-{[8-bromo-2-(1-methyl-1H-pyrazol-4-yl)[1,2,4]triazolo[1,5- |
| | c]quinazolin-5-yl]amino}azepan-2-one |
| | LC-MS (Method 2): Rt = 1.21 min; MS (ESIpos): m/z = 455 [M + H]⁺ |
| | ¹H-NMR (400 MHz, DMSO-d₆): δ [ppm] = 1.27-1.42 (m, 2H), 1.48-1.60 (m, |
| | 1H), 1.81-1.90 (m, 2H), 1.96-2.06 (m, 1H), 2.28 (br d, 1H), 3.16 (dt, 1H), |
| | 3.95 (s, 3H), 4.80 (dd, 1H), 7.58 (dd, 1H), 7.71 (d, 1H), 7.83 (d, 1H), 8.06 (d, |
| | 1H), 8.16 (d, 1H), 8.21 (dd, 1H), 8.48 (s, 1H). |
| Example 231 | 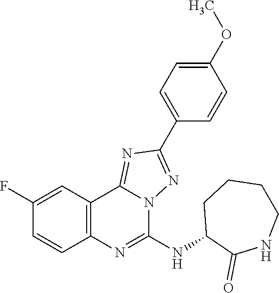 [SMILES: COc1ccc(-c2nc3c4cc(F)ccc4nc(N[C@@H]4CCCCNC4=O)n3n2)cc1] |
| | (3R)-3-{[9-fluoro-2-(4-methoxyphenyl)[1,2,4]triazolo[1,5-c]quinazolin-5- |
| | yl]amino}azepan-2-one |
| | LC-MS (Method 2): Rₜ = 1.38 min; MS (ESIneg): m/z = 420 [M − H]⁻ |
| | ¹H-NMR (400 MHz, DMSO-d₆): δ [ppm] = 1.23-1.43 (m, 1H), 1.49-1.66 (m, |
| | 1H), 1.77-1.95 (m, 2H), 1.97-2.11 (m, 1H), 2.24-2.36 (m, 1H), 3.07-3.23 |
| | (m, 1H), 3.34-3.42 (m, 1H), 3.86 (s, 3H), 4.80 (dd, 1H), 7.10-7.17 (m, 2H), |
| | 7.57-7.65 (m, 1H), 7.67-7.76 (m, 2H), 7.98 (dd, 1H), 8.16-8.27 (m, 3H). |
| Example 232 | 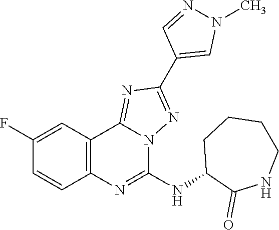 [SMILES: Cn1cc(-c2nc3c4cc(F)ccc4nc(N[C@@H]4CCCCNC4=O)n3n2)cn1] |
| | (3R)-3-{[9-fluoro-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.09 min; MS (ESIneg): m/z = 393 [M − H]⁻ |
| | ¹H-NMR (400 MHz, DMSO-d₆): δ [ppm] = 1.24-1.41 (m, 1H), 1.45-1.62 (m, |
| | 1H), 1.79-1.95 (m, 2H), 1.97-2.07 (m, 1H), 2.23-2.35 (m, 1H), 3.11-3.22 |
| | (m, 1H), 3.34-3.44 (m, 1H), 3.95 (s, 3H), 4.79 (dd, 1H), 7.56-7.64 (m, 2H), |
| | 7.69 (dd, 1H), 7.92 (dd, 1H), 8.06 (d, 1H), 8.22 (dd, 1H), 8.48 (s, 1H). |
| Example 233 | 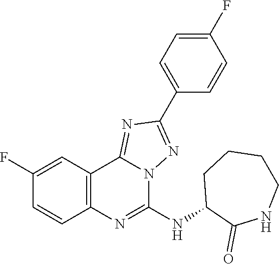 [SMILES: O=C1NCCCC[C@H]1Nc1nc2ccc(F)cc2c2nc(-c3ccc(F)cc3)nn12] |
| | (3R)-3-{[9-fluoro-2-(4-fluorophenyl)[1,2,4]triazol[1,5-c]quinazolin-5- |
| | yl]amino}azepan-2-one |
| | LC-MS (Method 2): Rₜ = 1.40 min; MS (ESIpos): m/z = 409 [M + H]⁺ |
| | ¹H-NMR (400 MHz, DMSO-d₆): δ [ppm] = 1.25-1.40 (m, 1H), 1.49-1.62 (m, |
| | 1H), 1.78-1.95 (m, 2H), 1.98-2.08 (m, 1H), 2.27-2.34 (m, 1H), 3.10-3.25 |
| | (m, 1H), 3.35-3.42 (m, 1H), 4.81 (dd, 1H), 7.38-7.50 (m, 2H), 7.58-7.66 |
| | (m, 1H), 7.68-7.77 (m, 2H), 8.00 (dd, 1H), 8.22 (dd, 1H), 8.28-8.37 (m, |
| | 2H). |
| Example 234 | 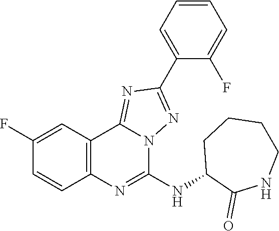 [SMILES: O=C1NCCCC[C@H]1Nc1nc2ccc(F)cc2c2nc(-c3ccccc3F)nn12] |
| | (3R)-3-{[9-fluoro-2-(2-fluorophenyl)[1,2,4]triazolo[1,5-c]quinazolin-5- |
| | yl]amino}azepan-2-one |
| | LC-MS (Method 2): Rₜ = 1.35 min; MS (ESIpos): m/z = 409 [M + H]⁺ |
| | ¹H-NMR (400 MHz, DMSO-d₆): δ [ppm] = 1.25-1.39 (m, 1H), 1.48-1.61 (m, |
| | 1H), 1.80-1.94 (m, 2H), 1.98-2.06 (m, 1H), 2.33 (ddd, 1H), 3.10-3.21 (m, |
| | 1H), 3.34-3.41 (m, 1H), 4.75-4.85 (m, 1H), 7.41-7.52 (m, 2H), 7.59-7.67 |
| | (m, 2H), 7.70-7.76 (m, 2H), 8.00 (dd, 1H), 8.19-8.30 (m, 2H). |
| Example 235 | 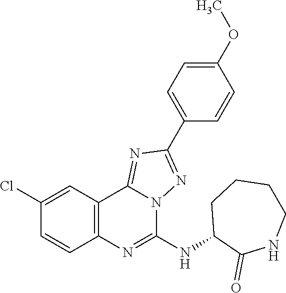 [SMILES: COc1ccc(-c2nc3c4cc(Cl)ccc4nc(N[C@@H]4CCCCNC4=O)n3n2)cc1] |
| | (3R)-3-{[9-chloro-2-(4-methoxyphenyl)[1,2,4]triazolo[1,5-c]quinazolin-5- |
| | yl]amino}azepan-2-one |
| | LC-MS (Method 2): Rₜ = 1.48 min; MS (ESIpos): m/z = 437 [M + H]⁺ |
| | ¹H-NMR (400 MHz, DMSO-d₆): δ [ppm] = 1.25-1.42 (m, 1H), 1.45-1.64 (m, |
| | 1H), 1.78-1.95 (m, 2H), 1.96-2.09 (m, 1H), 2.23-2.38 (m, 1H), 3.11-3.24 |
| | (m, 1H), 3.35-3.42 (m, 1H), 3.86 (s, 3H), 4.82 (dd, 1H), 7.10-7.18 (m, 2H), |
| | 7.62-7.69 (m, 1H), 7.70-7.76 (m, 1H), 7.79 (d, 1H), 8.17-8.26 (m, 4H). |
| Example 236 | 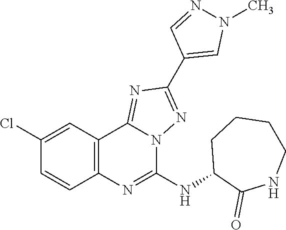 [SMILES: Cn1cc(-c2nc3c4cc(Cl)ccc4nc(N[C@@H]4CCCCNC4=O)n3n2)cn1] |
| | (3R)-3-{[9-chloro-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.17 min; MS (ESIpos): m/z = 411 [M + H]⁺ |
| | ¹H-NMR (400 MHz, DMSO-d₆): δ [ppm] = 1.25-1.38 (m, 1H), 1.47-1.60 (m, |
| | 1H), 1.78-1.94 (m, 2H), 1.96-2.08 (m, 1H), 2.29 (br d, 1H), 3.09-3.21 (m, |
| | 1H), 4.81 (br dd, 1H), 7.62-7.70 (m, 2H), 7.70-7.75 (m, 1H), 8.07 (d, 1H), |
| | 8.17 (d, 1H), 8.22 (dd, 1H), 8.48 (s, 1H). |
| Example 237 | 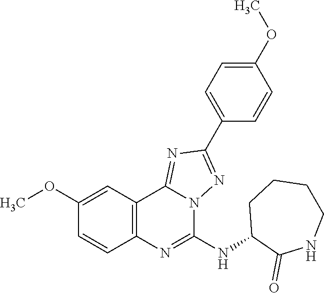 [SMILES: COc1ccc(-c2nc3c4cc(OC)ccc4nc(N[C@@H]4CCCCNC4=O)n3n2)cc1] |
| | (3R)-3-{[9-methoxy-2-(4-methoxyphenyl)[1,2,4]triazolo[1,5-c]quinazolin- |
| | 5-yl]amino}azepan-2-one |
| | LC-MS (Method 2): Rₜ = 1.30 min; MS (ESIpos): m/z = 433 [M + H]⁺ |
| | ¹H-NMR (400 MHz, DMSO-d₆): δ [ppm] = 1.24-1.41 (m, 1H), 1.46-1.64 (m, |
| | 1H), 1.76-1.94 (m, 2H), 2.00 (br s, 1H), 2.23-2.38 (m, 1H), 3.09-3.22 (m, |
| | 1H), 3.55-3.42 (m, 1H), 3.86 (s, 3H), 3.93 (s, 3H), 4.79 (dd, 1H), 7.10-7.17 |
| | (m, 2H), 7.35 (dd, 1H), 7.55 (d, 1H), 7.61 (d, 1H), 7.66 (d, 1H), 8.15-8.27 |
| | (m, 3H). |
| Example 238 | 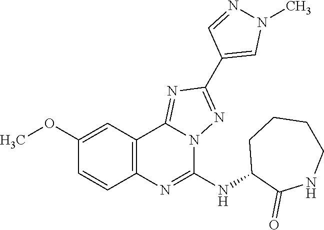 [SMILES: COc1ccc2nc(N[C@@H]3CCCCNC3=O)n3nc(-c4cnn(C)c4)nc3c2c1] |
| | (3R)-3-{[9-methoxy-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.04 min; MS (ESIpos): m/z = 407 [M + H]⁺ |
| | ¹H-NMR (400 MHz, DMSO-d₆): δ [ppm] = 1.24-1.39 (m, 1H), 1.46-1.60 (m, |
| | 1H), 1.78-1.94 (m, 2H), 1.96-2.06 (m, 1H), 2.24-2.35 (m, 1H), 3.09-3.23 |
| | (m, 1H), 3.35-3.40 (m, 1H), 3.91 (s, 3H), 3.95 (s, 3H), 4.78 (dd, 1H), 7.31- |
| | 7.36 (m, 1H), 7.45 (d, 1H), 7.60 (dd, 2H), 8.07 (s, 1H), 8.19 (dd, 1H), 8.50 (s, |
| | 1H). |
| Example 239 | 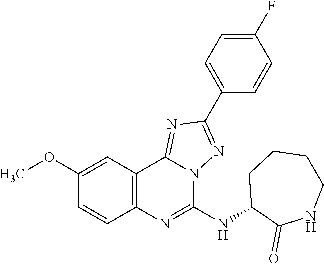 [SMILES: COc1ccc2nc(N[C@@H]3CCCCNC3=O)n3nc(-c4ccc(F)cc4)nc3c2c1] |
| | (3R)-3-{[2-(4-fluorophenyl)-9-methoxy[1,2,4]triazolo[1,5-c]quinazolin-5- |
| | yl]amino}azepan-2-one |
| | LC-MS (Method 2): Rₜ = 1.35 min; MS (ESIpos): m/z = 421 [M + H]⁺ |
| | ¹H-NMR (400 MHz, DMSO-d₆): δ [ppm] = 1.20-1.42 (m, 1H), 1.47-1.64 (m, |
| | 1H), 1.79-1.96 (m, 2H), 1.98-2.07 (m, 1H), 2.25-2.35 (m, 1H), 3.06-3.24 |
| | (m, 1H), 3.37-3.46 (m, 1H), 3.93 (s, 3H), 4.80 (br dd, 1H), 7.37 (dd, 1H), |
| | 7.43 (t, 2H), 7.57-7.64 (m, 2H), 7.67 (d, 1H), 8.20 (br dd, 1H), 8.29-8.38 |
| | (m, 2H). |
| Example 240 | 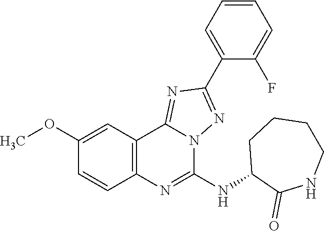 [SMILES: COc1ccc2nc(N[C@@H]3CCCCNC3=O)n3nc(-c4ccccc4F)nc3c2c1] |
| | (3R)-3-{[2-(2-fluorophenyl)-9-methoxy[1,2,4]triazolo[1,5-c]quinazolin-5- |
| | yl]amino}azepan-2-one |
| | LC-MS (Method 2): Rₜ = 1.29 min; MS (ESIpos): m/z = 421 [M + H]⁺ |
| | ¹H-NMR (400 MHz, DMSO-d₆): δ [ppm] = 1.25-1.40 (m, 1H), 1.48-1.61 (m, |
| | 1H), 1.79-1.95 (m, 2H), 1.98-2.07 (m, 1H), 2.27-2.36 (m, 1H), 3.09-3.23 |
| | (m, 1H), 3.93 (s, 3H), 4.80 (dd, 1H), 7.38 (dd, 1H), 7.41-7.49 (m, 2H), 7.58 |
| | (d, 1H), 7.60-7.69 (m, 3H), 8.21 (dd, 1H), 8.28 (td, 1H). |
| Example 241 | 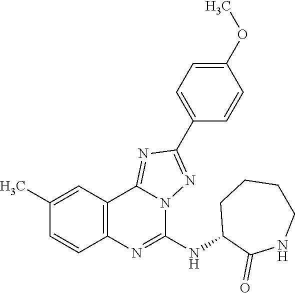 [SMILES: COc1ccc(-c2nc3c4cc(C)ccc4nc(N[C@@H]4CCCCNC4=O)n3n2)cc1] |
| | (3R)-3-{[2-(4-methoxyphenyl)-9-methyl[1,2,4]triazolo[1,5-c]quinazolin-5- |
| | yl]amino}azepan-2-one |
| | LC-MS (Method 2): Rₜ = 1.38 min; MS (ESIpos): m/z = 417 [M + H]⁺ |
| | ¹H-NMR (400 MHz, DMSO-d₆): δ [ppm] = .25-1.39 (m, 1H), 1.48-1.62 (m, |
| | 1H), 1.78-1.95 (m, 2H), 1.97-2.08 (m, 1H), 2.31 (br d, 1H), 3.09-3.21 (m, |
| | 1H), 3.34-3.41 (m, 1H), 3.86 (s, 3H), 4.76-4.85 (m, 1H), 7.11-7.17 (m, |
| | 2H), 7.52-7.59 (m, 2H), 7.63 (d, 1H), 8.08-8.11 (m, 1H), 8.17-8.24 (m, |
| | 3H). |
| Example 242 | 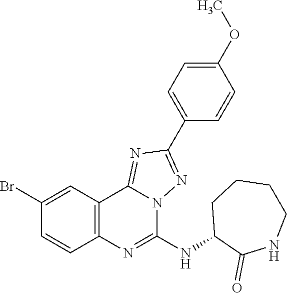 [SMILES: COc1ccc(-c2nc3c4cc(Br)ccc4nc(N[C@@H]4CCCCNC4=O)n3n2)cc1] |
| | (3R)-3-{[9-bromo-2-(4-methoxypheny)[1,2,4]triazolo[1,5-c]quinazolin-5- |
| | yl]amino}azepan-2-one |
| | LC-MS (Method 2): Rₜ = 1.50 min; MS (ESIpos): m/z = 481 [M + H]⁺ |
| | ¹H-NMR (400 MHz, DMSO-d₆): δ [ppm] = 1.26-1.40 (m, 1H), 1.50-1.64 (m, |
| | 1H), 1.80-1.92 (m, 2H), 1.97-2.08 (m, 1H), 2.29 (br s, 1H), 3.11-3.20 (m, |
| | 1H), 3.36-3.41 (m, 1H), 4.81 (dd, 1H), 7.12-7.17 (m, 2H), 7.59 (d, 1H), |
| | 7.80 (d, 1H), 7.84 (dd, 1H), 8.20-8.23 (m, 3H), 8.36 (d, 1H). |
| Example 243 | 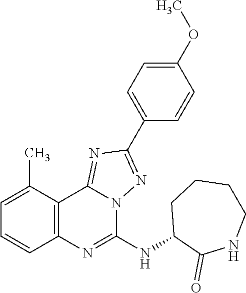 [SMILES: COc1ccc(-c2nc3c4c(C)cccc4nc(N[C@@H]4CCCCNC4=O)n3n2)cc1] |
| | (3R)-3-{[2-(4-methoxyphenyl)-10-methyl[1,2,4]triazolo[1,5-c]quinazolin- |
| | 5-yl]amino}azepan-2-one |
| | LC-MS (Method 2): Rₜ = 1.51 min; MS (ESIpos): m/z = 417 [M + H]⁺ |
| | ¹H-NMR (400 MHz, DMSO-d₆): δ [ppm] = 1.26-1.39 (m, 1H), 1.49-1.62 (m, |
| | 1H), 1.80-1.94 (m, 2H), 1.97-2.07 (m, 1H), 2.28-2.35 (m, 1H), 3.02 (s, |
| | 3H), 3.11-3.21 (m, 1H), 3.34-3.41 (m, 1H), 3.86 (s, 3H), 4.79-4.86 (m, |
| | 1H), 7.12-7.18 (m, 2H), 7.24-7.29 (m, 1H), 7.47-7.52 (m, 1H), 7.56-7.62 |
| | (m, 1H), 7.69 (d, 1H), 8.18-8.26 (m, 3H). |
| Example 244 | 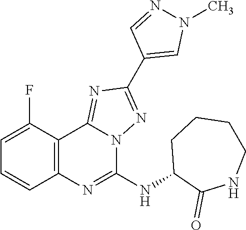 [SMILES: Cn1cc(-c2nc3c4c(F)cccc4nc(N[C@@H]4CCCCNC4=O)n3n2)cn1] |
| | (3R)-3-{[10-fluoro-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.01 min; MS (ESIpos): m/z = 395 [M + H]⁺ |
| | ¹H-NMR (400 MHz, DMSO-d₆): δ [ppm] = 1.25-1.38 (m, 1H), 1.48-1.61 (m, |
| | 1H), 1.85 (td, 2H), 1.97-2.06 (m, 1H), 2.29 (br d, 1H), 3.10-3.21 (m, 1H), |
| | 3.38 (td, 1H), 3.95 (s, 3H), 4.77-4.86 (m, 1H), 7.24 (ddd, 1H), 7.47 (dd, 1H), |
| | 7.66-7.71 (m, 1H), 7.73 (d, 1H), 8.06 (s, 1H), 8.22 (dd, 1H), 8.50 (s, 1H). |
| Example 245 | 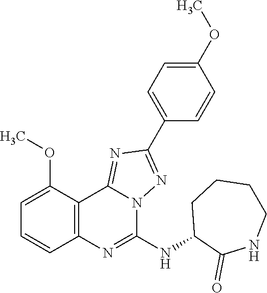 [SMILES: COc1ccc(-c2nc3c4c(OC)cccc4nc(N[C@@H]4CCCCNC4=O)n3n2)cc1] |
| | (3R)-3-{[10-methoxy-2-(4-methoxyphenyl)[1,2,4]triazolo[1,5- |
| | c]quinazolin-5-yl]amino}azepan-2-one |
| | LC-MS (Method 2): Rₜ = 1.19 min; MS (ESIpos): m/z = 433 [M + H]⁺ |
| | ¹H-NMR (400 MHz, DMSO-d₆): δ [ppm] = 1.25-1.39 (m, 1H), 1.49-1.61 (m, |
| | 1H), 1.80-1.94 (m, 2H), 1.97-2.06 (m, 1H), 2.24-2.35 (m, 1H), 3.11-3.21 |
| | (m, 1H), 3.34-3.41 (m, 1H), 3.86 (s, 3H), 4.03 (s, 3H), 4.81 (dd, 1H), 6.97- |
| | 7.02 (m, 1H), 7.11-7.17 (m, 2H), 7.23 (dd, 1H), 7.59-7.67 (m, 1H), 7.71 (d, |
| | 1H), 8.17-8.23 (m, 3H). |
| Example 246 | 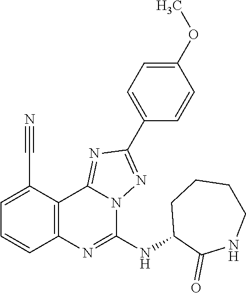 [SMILES: COc1ccc(-c2nc3c4c(C#N)cccc4nc(N[C@@H]4CCCCNC4=O)n3n2)cc1] |
| | 2-(4-methoxyphenyl)-5-{[(3R)-2-oxoazepan-3- |
| | yl]amino}[1,2,4]triazolo[1,5-c]quinazoline-10-carbonitrile |
| | LC-MS (Method 2): Rₜ = 1.26 min; MS (ESIpos): m/z = 428 [M + H]⁺ |
| | ¹H-NMR (400 MHz, DMSO-d₆): δ [ppm] = 1.27-1.43 (m, 1H), 1.52-1.65 (m, |
| | 1H), 1.77-1.95 (m, 2H), 1.98-2.08 (m, 1H), 2.29 (br d, 1H), 3.10-3.23 (m, |
| | 1H), 3.39 (br d, 1H), 3.87 (s, 3H), 4.84 (br dd, 1H), 7.14-7.22 (m, 2H), 7.80- |
| | 7.87 (m, 1H), 7.90-7.97 (m, 3H), 8.19-8.28 (m, 3H). |
| Example 247 | 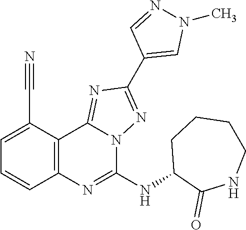 [SMILES: Cn1cc(-c2nc3c4c(C#N)cccc4nc(N[C@@H]4CCCCNC4=O)n3n2)cn1] |
| | 2-(1-methyl-1H-pyrazol-4-yl)-5-{[(3R)-2-oxoazepan-3- |
| | yl]amino}[1,2,4]triazolo[1,5-c]quinazoline-10-carbonitrile |
| | LC-MS (Method 2): Rₜ = 1.04 min; MS (ESIpos): m/z = 402 [M + H]⁺ |
| | ¹H-NMR (400 MHz, DMSO-d₆): δ [ppm] = 1.26-1.39 (m, 1H), 1.49-1.63 (m, |
| | 1H), 1.79-1.94 (m, 2H), 1.98-2.06 (m, 1H), 2.26-2.35 (m, 1H), 3.11-3.21 |
| | (m, 1H), 3.35-3.40 (m, 1H), 3.97 (s, 3H), 4.83 (dd, 1H), 7.79-7.86 (m, 2H), |
| | 7.93 (s, 1H), 7.94-7.96 (m, 1H), 8.05 (d, 1H), 8.24 (dd, 1H), 8.46 (s, 1H). |
| Example 248 | 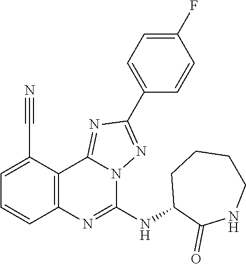 [SMILES: N#Cc1cccc2nc(N[C@@H]3CCCCNC3=O)n3nc(-c4ccc(F)cc4)nc3c12] |
| | 2-(4-fluorophenyl)-5-{[(3R)-2-oxoazepan-3-yl]amino}[1,2,4]triazolo[1,5- |
| | c]quinazoline-10-carbonitrile |
| | LC-MS (Method 2): Rₜ = 1.29 min; MS (ESIpos): m/z = 416 [M + H]⁺ |
| | ¹H-NMR (400 MHz, DMSO-d₆): δ [ppm] = 1.25-1.41 (m, 1H), 1.51-1.67 (m, |
| | 1H), 1.81-1.94 (m, 2H), 1.98-2.07 (m, 1H, 2.30 (br s, 1H), 3.11-3.22 (m, |
| | 1H), 3.353-3.43 (m, 1H), 4.85 (br dd, 1H), 7.48 (t, 2H), 7.80-7.90 (m, 1H), |
| | 7.92-8.00 (m, 3H), 8.20-8.29 (m, 1H), 8.30-8.39 (m, 2H). |
| Example 249 | 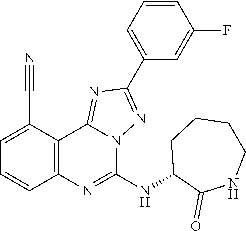 [SMILES: N#Cc1cccc2nc(N[C@@H]3CCCCNC3=O)n3nc(-c4cccc(F)c4)nc3c12] |
| | 2-(3-fluorophenyl)-5-{[(3R)-2-oxoazepan-3-yl]amino}[1,2,4]triazolo[1,5- |
| | c]quinazoline-10-carbonitrile |
| | LC-MS (Method 2): Rₜ = 1.29 min; MS (ESIpos): m/z = 416 [M + H]⁺ |
| | ¹H-NMR (400 MHz, DMSO-d₆): δ [ppm] = 1.26-1.41 (m, 1H), 1.53-1.66 (m, |
| | 1H), 1.80-1.91 (m, 2H), 1.98-2.08 (m, 1H), 2.26-2.32 (m, 1H), 3.12-3.23 |
| | (m, 1H), 3.35 (br d, 1H), 4.85 (br dd, 1H), 7.42-7.50 (m, 1H), 7.69 (td, 1H), |
| | 7.81-7.88 (m, 1H), 7.90-8.01 (m, 4H), 8.14 (dt, 1H), 8.25 (dd, 1H). |

### Example 250

### Methyl 2-(4-methoxyphenyl)-5-{[(3R)-2-oxoazepan-3-yl]amino}[1,2,4]triazolo[1,5-c]quinazoline-7-carboxylate

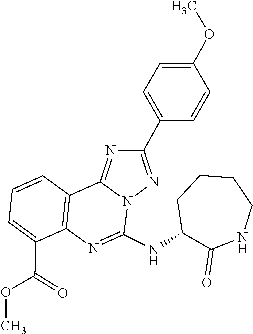 [SMILES: COC(=O)c1cccc2c1nc(N[C@@H]1CCCCNC1=O)n1nc(-c3ccc(OC)cc3)nc21]

(3R)-3-{[7-Bromo-2-(4-methoxyphenyl)[1,2,4]triazolo[1,5-c]quinazolin-5-yl]amino}azepan-2-one (176 mg, 366 μmol) was suspended in methanol/THF (11 mL, 10:1) in an autoclave (50 mL). 1,1′-Bis(diphenylphosphino)ferrocene-palladium(II)dichloride dichloromethane complex (30 mg, 36.6 μmol) and triethylamine (102 μL, 731 μmol) were added. The reaction mixture was purged three times with carbon monoxide at rt. Then, the autoclave was filled with carbon monoxide up to 12.11 bar and it was stirred for 30 min at rt. As the pressure was constant at 11.53 bar the carbon monoxide was released and the autoclave was evacuated under vacuum. The autoclave was filled with carbon monoxide up to 16.04 bar at 20° C. internal temperature. The reaction mixture was stirred for 24 h at 100° C. internal temperature. The reaction mixture was allowed to cool down to rt and the carbon monoxide was removed. The reaction mixture was concentrated and digested in ethyl acetate. The insoluble residue was filtered off, washed with ethyl acetate and the filtrate was concentrated under reduce pressure affording 210 mg of the title product.

LC-MS (Method 2): Rₜ=1.27 min; MS (ESIpos): m/z=461 [M+H]⁺

¹H-NMR (400 MHz, DMSO-d₆): δ [ppm]=1.28-1.38 (m, 1H), 1.47-1.59 (m, 1H), 1.76-1.91 (m, 2H), 1.99-2.09 (m, 1H), 2.40 (br d, 1H), 3.13-3.23 (m, 1H), 3.23-3.31 (m, 1H), 3.86 (s, 3H), 3.93 (s, 3H), 4.71-4.77 (m, 1H), 7.13-7.17 (m, 2H), 7.49 (t, 1H), 7.84 (d, 1H), 7.94 (dd, 1H), 8.21-8.26 (m, 3H), 8.43 (dd, 1H).

### Example 251

### (3R)-3-{[8-Cyclopropyl-2-(4-methoxyphenyl)[1,2,4]triazolo[1,5-c]quinazolin-5-yl]amino}azepan-2-one

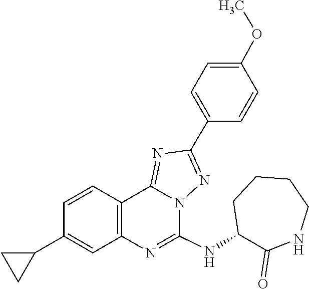

(3R)-3-{[8-Bromo-2-(4-methoxyphenyl)[1,2,4]triazolo[1,5-c]quinazolin-5-yl]amino}azepan-2-one (27.6 mg, 57.3 μmol), cyclopropylboronic acid (5.91 mg, 68.8 μmol), bis(diphenylphosphino)ferrocene)dichlorpalladium(II)*dichlormethane complex (9.36 mg, 11.5 μmol), caesium carbonate (74.7 mg, 229 μmol) were suspended in 1,4-dioxane and stirred for 10 min at 130° C. under microwave irradiation. The reaction mixture was then filtered, washed with dioxane and dried under reduced pressure. The crude material was purified by preparative HPLC to give 8.00 mg (96% purity, 30% yield) of the title compound.

LC-MS (Method 2): Rₜ=1.44 min; MS (ESIpos): m/z=443 [M+H]⁺

1H-NMR (400 MHz, DMSO-d₆): δ [ppm]=0.78-0.92 (m, 2H), 1.02-1.12 (m, 2H), 1.17-1.43 (m, 2H), 1.46-1.69 (m, 1H), 1.81-1.95 (m, 2H), 1.98-2.15 (m, 2H), 2.26-2.34 (m, 1H), 3.11-3.22 (m, 1H), 3.85 (s, 3H), 4.81 (br dd, 1H), 7.10-7.17 (m, 3H), 7.33 (d, 1H), 7.65 (d, 1H), 8.12-8.23 (m, 4H).

### Example 252

### methyl 2-(4-methoxyphenyl)-5-{[(3R)-2-oxoazepan-3-yl]amino}[1,2,4]triazolo[1,5-c]quinazoline-10-carboxylate

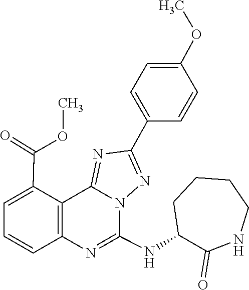 [SMILES: COC(=O)c1cccc2nc(N[C@@H]3CCCCNC3=O)n3nc(-c4ccc(OC)cc4)nc3c12]

Methyl 5-chloro-2-(4-methoxyphenyl)[1,2,4]triazolo[1,5-c]quinazoline-10-carboxylate (14.5 mg, 39.3 μmol), (3R)-3-aminoazepan-2-one (5.54 mg, 43.3 μmol) and N,N-diisopropylethylamine (21 μl, 120 μmol) were stirred in DMSO (270 μl) for 2 h at 60° C. Water was added to the mixture, and the solid was filtered, washed with water and dried under reduced pressure at 60° C. The solid was purified by preparative TLC and then suspended in DMSO, filtered and dried under reduced pressure to give 2.10 mg (95% purity, 11% yield) of the title compound LC-MS (method 2): Rₜ=1.29 min; MS (ESIpos): m/z=461 [M+H]⁺

¹H-NMR (400 MHz, DMSO-d6): δ [ppm]=1.26-1.40 (m, 1H), 1.51-1.64 (m, 1H), 1.80-1.95 (m, 2H), 1.97-2.08 (m, 1H), 2.28-2.36 (m, 1H), 3.11-3.22 (m, 1H), 3.34-3.42 (m, 1H), 3.86 (s, 3H), 4.02 (s, 3H), 4.84 (dd, 1H), 7.11-7.19 (m, 2H), 7.47 (dd, 1H), 7.74-7.77 (m, 2H), 7.81 (d, 1H), 8.14-8.20 (m, 2H), 8.22 (dd, 1H).

### Example 253

### (6R)-6-{[7-Chloro-2-(1-methyl-1H-pyrazol-4-yl)[1,2,4]triazolo[1,5-c]quinazolin-5-yl]amino}-1,4-diazepan-5-one

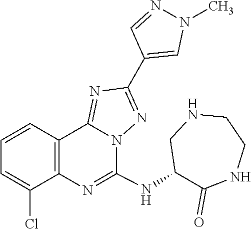 [SMILES: Cn1cc(-c2nc3c4cccc(Cl)c4nc(N[C@@H]4CNCCNC4=O)n3n2)cn1]

Benzyl (6R)-6-{[7-chloro-2-(1-methyl-1H-pyrazol-4-yl)[1,2,4]triazolo[1,5-c]quinazolin-5-yl]amino}-5-oxo-1,4-diazepane-1-carboxylate (96.1 mg, 176 μmol) was solubilised in dichloromethane (3.0 mL), hydrobromic acid in acetic acid (310 μL, 33%, 1.8 mmol) was added and the mixture was stirred for 2 h at rt. The suspension was diluted with ethyl acetate and filtered. The solid was stirred in methanol/dichloromethane and basified with triethylamine. The resulting suspension was filtered, washed with methanol and dried under reduced pressure at 40° C. to give 7.50 mg (95% purity, 10% yield) of the title compound.

LC-MS (method 2): Rₜ=0.86 min; MS (ESIpos): m/z=412 [M+H]⁺

¹H-NMR (400 MHz, DMSO-d₆): δ [ppm]=2.65-2.73 (m, 2H), 3.04 (br dd, 1H), 3.07-3.18 (m, 1H), 3.37-3.47 (m, 1H), 3.51 (dd, 1H), 3.95 (s, 3H), 4.89 (ddd, 1H), 7.40 (t, 1H), 7.75 (d, 1H), 7.89 (dd, 1H), 8.07 (s, 1H), 8.21 (dd, 1H), 8.30 (dd, 1H), 8.50 (s, 1H).

The following compounds were synthesised analogously to example 253:

| | Structure |
|---|---|
| | IUPAC-Name |
| | LC-MS (method): Retention time; Mass found |
| Example | ¹H-NMR |
| Example 254 | 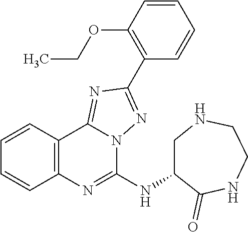 [SMILES: CCOc1ccccc1-c1nc2c3ccccc3nc(N[C@@H]3CNCCNC3=O)n2n1] |
| | (6R)-6-{[2-(2-ethoxyphenyl)[1,2,4]triazolo[1,5-c]quinazolin-5-yl]amino}- |
| | 1,4-diazepan-5-one |
| | LC-MS (Method 2): Rₜ = 1.06 min; MS (ESIpos): m/z = 418 [M + H]⁺ |
| | ¹H-NMR (400 MHz, DMSO-d₆): δ [ppm] = 1.45 (t, 3H), 2.63 (dd, 1H), 2.94 |
| | (br d, 1H), 3.02 (br dd, 1H), 3.07-3.16 (m, 1H), 3.38-3.50 (m, 2H), 4.13-4.22 |
| | (m, 2H), 4.85 (ddd, 1H), 7.13 (td, 1H), 7.22 (d, 1H), 7.46 (ddd, 1H), 7.48- |
| | 7.54 (m, 1H), 7.67-7.71 (m, 1H), 7.72-7.78 (m, 2H), 8.09 (dd, 1H), 8.29 |
| | (dd, 1H), 8.30-8.35 (m, 1H). |
| | The reaction was stirred 3.5 h at 0° C. and then 18 h at rt. |
| Example 255 | 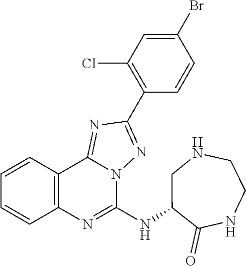 [SMILES: O=C1NCCNC[C@H]1Nc1nc2ccccc2c2nc(-c3ccc(Br)cc3Cl)nn12] |
| | (6R)-6-{[2-(4-bromo-2-chlorophenyl)[1,2,4]triazolo[1,5-c]quinazolin-5- |
| | yl]amino}-1,4-diazepan-5-one |
| | LC-MS (Method 2): Rₜ = 1.24 min; MS (ESIpos): m/z = 486 [M + H]⁺ |
| | ¹H-NMR (400 MHz, DMSO-d₆): δ [ppm] = 2.62-2.70 (m, 1H), 3.02 (br dd, |
| | 1H), 3.06-3.16 (m, 1H), 3.37-3.48 (m, 2H), 4.87 (ddd, 1H), 7.47 (ddd, 1H), |
| | 7.68-7.73 (m, 2H), 7.74-7.81 (m, 2H), 8.00 (d, 1H), 8.05 (d, 1H), 8.26- |
| | 8.32 (m, 2H). |
| | The reaction was stirred 3.5 h at 0° C. and then 18 h at rt. |
| Example 256 | 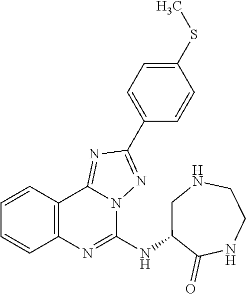 [SMILES: CSc1ccc(-c2nc3c4ccccc4nc(N[C@@H]4CNCCNC4=O)n3n2)cc1] |
| | (6R)-6-({2-[4-(methylsulfanyl)phenyl][1,2,4]triazolo[1,5-c]quinazolin-5- |
| | yl}amino)-1,4-diazepan-5-one |
| | LC-MS (Method 2): Rₜ = 1.16 min; MS (ESIpos): m/z = 420 [M + H]⁺ |
| | ¹H-NMR (400 MHz, DMSO-d₆): δ [ppm] = 2.56 (s, 3H), 2.66-2.76 (m, 1H), |
| | 2.93-3.08 (m, 3H), 3.08-3.17 (m, 1H), 3.40-3.49 (m, 2H), 4.90 (ddd, 1H), |
| | 7.43-7.49 (m, 3H), 7.67-7.72 (m, 2H), 7.73-7.79 (m, 1H), 8.19-8.23 (m, |
| | 2H), 8.28-8.34 (m, 2H). |
| Example 257 | 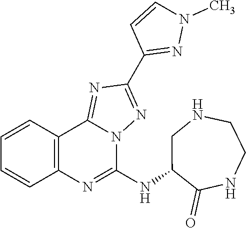 [SMILES: Cn1ccc(-c2nc3c4ccccc4nc(N[C@@H]4CNCCNC4=O)n3n2)n1] |
| | (6R)-6-{[2-(1-methyl-1H-pyrazol-3-yl)[1,2,4]triazolo[1,5-c]quinazolin-5- |
| | yl]amino}-1,4-diazepan-5-one |
| | LC-MS (Method 2): Rt = 0.77 min; MS (ESIpos): m/z = 378 [M + H]⁺ |
| | ¹H-NMR (400 MHz, DMSO-d₆): δ [ppm] = 2.64-2.70 (m, 1H), 2.84-2.97 (m, |
| | 1H), 2.97-3.15 (m, 2H), 3.38-3.48 (m, 2H), 3.99 (s, 3H), 4.87 (ddd, 1H), |
| | 6.92 (d, 1H), 7.46 (ddd, 1H), 7.61-7.71 (m, 2H), 7.72-7.79 (m, 1H), 7.89 |
| | (d, 1H), 8.23-8.36 (m, 2H). |
| Example 258 | 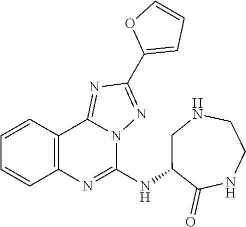 [SMILES: O=C1NCCNC[C@H]1Nc1nc2ccccc2c2nc(-c3ccco3)nn12] |
| | (6R)-6-{[2-(furan-2-yl)[1,2,4]triazolo[1,5-c]quinazolin-5-yl]amino}-1,4- |
| | diazepan-5-one |
| | LC-MS (Method 2): Rt = 0.90 min; MS (ESIpos): m/z = 364 [M + H]⁺ |
| | ¹H-NMR (400 MHz, DMSO-d₆): δ [ppm] = 2.52-2.54 (m, 1H), 2.62-2.70 (m, |
| | 1H), 2.93 (br s, 1H), 3.02 (br dd, 1H), 3.06-3.15 (m, 1H), 3.36-3.46 (m, |
| | 2H), 4.86 (ddd, 1H), 6.75-6.77 (m, 1H), 7.32 (dd, 1H), 7.46 (ddd, 1H), 7.65 |
| | (d, 1H), 7.67-7.71 (m, 1H), 7.73-7.78 (m, 1H), 7.98 (dd, 1H), 8.27 (d, 1H), |
| | 8.31 (t, 1H). |
| Example 259 | 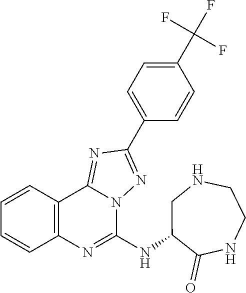 [SMILES: O=C1NCCNC[C@H]1Nc1nc2ccccc2c2nc(-c3ccc(C(F)(F)F)cc3)nn12] |
| | (6R)-6-({2-[4-(trifluoromethyl)phenyl][1,2,4]triazolo[1,5-c]quinazolin-5- |
| | yl}amino)-1,4-diazepan-5-one |
| | LC-MS (Method 2): Rt = 1.22 min; MS (ESIpos): m/z = 442 [M + H]⁺ |
| | ¹H-NMR (400 MHz, DMSO-d₆): δ [ppm] = 2.67-2.74 (m, 1H), 3.03 (br dd, |
| | 1H), 3.06-3.16 (m, 1H), 3.38-3.48 (m, 2H), 4.89 (ddd, 1H), 7.48 (ddd, 1H), |
| | 7.69-7.73 (m, 1H), 7.73-7.80 (m, 2H), 7.96-8.00 (m, 2H), 8.29-8.35 (m, |
| | 2H), 8.48-8.52 (m, 2H). |
| | The reaction was stirred 3 h at 0° C. |
| Example 260 | 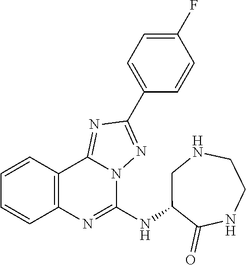 [SMILES: O=C1NCCNC[C@H]1Nc1nc2ccccc2c2nc(-c3ccc(F)cc3)nn12] |
| | (6R)-6-{[2-(4-fluorophenyl)[1,2,4]triazolo[1,5-c]quinazolin-5-yl]amino}- |
| | 1,4-diazepan-5-one |
| | LC-MS (Method 2): Rt = 1.08 min; MS (ESIpos): m/z = 392 [M + H]⁺ |
| | ¹H-NMR (400 MHz, DMSO-d₆): δ [ppm] = 2.52-2.55 (m, 1H), 2.64-2.73 (m, |
| | 1H), 2.86-2.96 (m, 1H), 3.03 (br dd, 1H), 3.07-3.15 (m, 1H), 3.36-3.47 |
| | (m, 2H), 4.85-4.91 (m, 1H), 7.41-7.49 (m, 3H), 7.67-7.78 (m, 3H), 8.28- |
| | 8.36 (m, 4H). |
| | The reaction was stirred 3 h at 0 °C. |
| Example 261 | 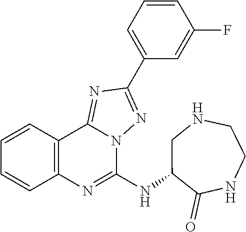 [SMILES: O=C1NCCNC[C@H]1Nc1nc2ccccc2c2nc(-c3cccc(F)c3)nn12] |
| | (6R)-6-{[2-(3-fluorophenyl)[1,2,4]triazolo[1,5-c]quinazolin-5-yl]amino}- |
| | 1,4-diazepan-5-one |
| | LC-MS (Method 2): Rt = 1.09 min; MS (ESIpos): m/z = 392 [M + H]⁺ |
| | ¹H-NMR (400 MHz, DMSO-d₆): δ [ppm] = 2.52-2.56 (m, 1H), 2.65-2.73 (m, |
| | 1H), 2.92 (br s, 1H), 3.03 (br dd, 1H), 3.07-3.16 (m, 1H), 3.36-3.47 (m, |
| | 2H), 4.86-4.92 (m, 1H), 7.40-7.50 (m, 2H), 7.63-7.79 (m, 4H), 7.97-8.02 |
| | (m, 1H), 8.13 (dt, 1H), 8.28-8.33 (m, 2H). |
| | The reaction was stirred 3 h at 0 °C. |
| Example 262 | 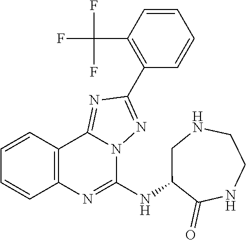 [SMILES: O=C1NCCNC[C@H]1Nc1nc2ccccc2c2nc(-c3ccccc3C(F)(F)F)nn12] |
| | (6R)-6-({2-[2-(trifluoromethyl)phenyl][1,2,4]triazolo[1,5-c]quinazolin-5- |
| | yl}amino)-1,4-diazepan-5-one |
| | LC-MS (Method 2): Rt = 1.09 min; MS (ESIpos): m/z = 442 [M + H]⁺ |
| | ¹H-NMR (400 MHz, DMSO-d₆): δ [ppm] = 2.61-2.69 (m, 1H), 2.91 (br s, 1H), |
| | 3.02 (br d, 1H), 3.05-3.14 (m, 1H), 3.36-3.47 (m, 2H), 4.82-4.89 (m, 1H), |
| | 7.48 (t, 1H), 7.67-7.75 (m, 2H), 7.75-7.81 (m, 1H), 7.82 (d, 1H), 7.85- |
| | 7.90 (m, 1H), 8.00 (t, 2H), 8.24-8.31 (m, 2H). |
| | The reaction was stirred 3.5 h at 0° C. and then 18 h at rt. |
| Example 263 | 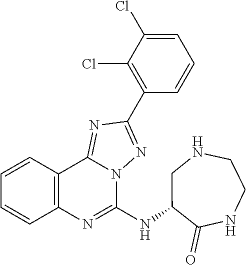 [SMILES: O=C1NCCNC[C@H]1Nc1nc2ccccc2c2nc(-c3cccc(Cl)c3Cl)nn12] |
| | (6R)-6-{[2-(2, 3-dichlorophenyl)[1,2,4]triazolo[1,5-c]quinazolin-5- |
| | yl]amino}-1,4-diazepan-5-one |
| | LC-MS (Method 2): Rₜ = 1.15 min; MS (ESIpos): m/z = 442 [M + H]⁺ |
| | ¹H-NMR (400 MHz, DMSO-d₆): δ [ppm] = 2.62-2.71 (m, 1H), 2.92 (br s, 1H), |
| | 3.02 (br dd, 1H), 3.07-3.16 (m, 1H), 3.38-3.48 (m, 2H), 4.87 (ddd, 1H), |
| | 7.48 (ddd, 1H), 7.58 (t, 1H), 7.69-7.74 (m, 2H), 7.75-7.81 (m, 1H), 7.88 |
| | (dd, 1H), 7.98 (dd, 1H), 8.27-8.33 (m, 2H). |
| Example 264 | 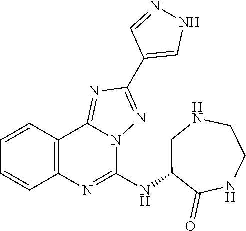 [SMILES: O=C1NCCNC[C@H]1Nc1nc2ccccc2c2nc(-c3cn[nH]c3)nn12] |
| | (6R)-6-{[2-(1H-pyrazol-4-yl)[1,2,4]triazolo[1,5-c]quinazolin-5-yl]amino}- |
| | 1,4-diazepan-5-one |
| | LC-MS (Method 2): Rt = 0.69 min; MS (ESIpos): m/z = 364 [M + H]⁺ |
| | ¹H-NMR (400 MHz, DMSO-d₆): δ [ppm] = 2.64-2.71 (m, 1H), 2.99-3.07 (m, |
| | 1H), 3.07-3.15 (m, 1H), 3.37-3.44 (m, 2H), 4.89 (ddd, 1H), 7.45 (ddd, 1H), |
| | 7.57 (d, 1H), 7.66-7.70 (m, 1H), 7.70-7.76 (m, 1H), 8.07-8.20 (m, 1H), |
| | 8.26 (dd, 1H), 8.30 (dd, 1H), 8.49 (br s, 1H), 13.36 (br s, 1H). |
| | The reaction was stirred 3.5 h at 0° C. and then 18 h at rt. |
| Example 265 | 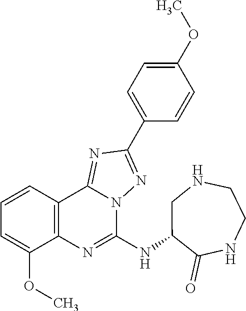 [SMILES: COc1ccc(-c2nc3c4cccc(OC)c4nc(N[C@@H]4CNCCNC4=O)n3n2)cc1] |
| | (6R)-6-{[7-methoxy-2-(4-methoxyphenyl)[1,2,4]triazolo[1,5-c]quinazolin- |
| | 5-yl]amino}-1,4-diazepan-5-one |
| | LC-MS (Method 2): Rt = 1.04 min; MS (ESIpos): m/z = 434 [M + H]⁺ |
| | ¹H-NMR (400 MHz, DMSO-d₆): δ [ppm] = 1.23 (s. 2H), 3.03 (br dd, 1H), 3.07- |
| | 3.18 (m, 1H), 3.37-3.50 (m, 2H), 3.85 (s, 3H), 3.96 (s, 3H), 4.88 (br dd, 1H), |
| | 7.11-7.17 (m, 2H), 7.27-7.33 (m, 1H), 7.34-7.42 (m, 1H), 7.67 (d, 1H), |
| | 7.87 (dd, 1H), 8.18-8.24 (m, 2H), 8.24-8.32 (m, 1H). |
| Example 266 | 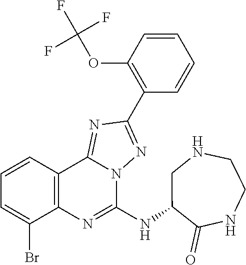 [SMILES: O=C1NCCNC[C@H]1Nc1nc2c(Br)cccc2c2nc(-c3ccccc3OC(F)(F)F)nn12] |
| | (6R)-6-({7-bromo-2-[2-(trifluoromethoxy)phenyl][1,2,4]triazolo[1,5- |
| | c]quinazolin-5-yl}amino)-1,4-diazepan-5-one |
| | LC-MS (Method 2): Rₜ = 1.29 min; MS (ESIpos): m/z = 536 [M + H]⁺ |
| | ¹H-NMR (400 MHz, DMSO-d₆): δ [ppm] = 2.64-2.71 (m, 2H), 3.04 (br dd, |
| | 1H), 3.13 (ddd, 1H), 3.42 (ddd, 1H), 3.61 (br d, 1H), 4.87 (br dd, 1H), 7.38 (t, |
| | 1H), 7.59-7.69 (m, 2H), 7.70-7.76 (m, 1H), 7.86 (d, 1H), 8.10 (dd, 1H), |
| | 8.27-8.32 (m, 2H), 8.39 (dd, 1H). |
| Example 267 | 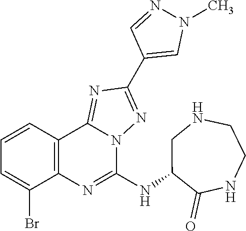 [SMILES: Cn1cc(-c2nc3c4cccc(Br)c4nc(N[C@@H]4CNCCNC4=O)n3n2)cn1] |
| | (6R)-6-{[7-bromo-2-(1-methyl-1H-pyrazol-4-yl)[1,2,4]triazolo[1,5- |
| | c]quinazolin-5-yl]amino}-1,4-diazepan-5-one |
| | LC-MS (Method 2): Rt = 0.88 min; MS (ESIpos): m/z = 456 [M + H]⁺ |
| | ¹H-NMR (400 MHz, DMSO-d₆): δ [ppm] = 2.64-2.73 (m, 2H), 2.98-3.16 (m, |
| | 2H), 3.50-3.63 (m, 1H), 3.95 (s, 3H), 4.83-4.95 (m, 1H), 7.22-7.41 (m, |
| | 1H), 7.68-7.76 (m, 1H), 8.00-8.10 (m, 2H), 8.19-8.36 (m, 2H), 8.44-8.52 |
| | (m, 1H). |
| Example 268 | 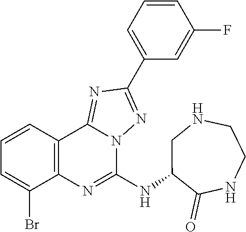 [SMILES: O=C1NCCNC[C@H]1Nc1nc2c(Br)cccc2c2nc(-c3cccc(F)c3)nn12] |
| | (6R)-6-{[7-bromo-2-(3-fluorophenyl)[1,2,4]triazolo[1,5-c]quinazolin-5- |
| | yl]amino}-1,4-diazepan-5-one |
| | LC-MS (Method 2): Rₜ = 1.21 min; MS (ESIpos): m/z = 470 [M + H]⁺ |
| | ¹H-NMR (400 MHz, DMSO-d₆): δ [ppm] = 2.53-2.60 (m, 1H), 2.76 (dd, 1H), |
| | 3.06 (br dd, 1H), 3.10-3.19 (m, 1H), 3.38-3.48 (m, 1H), 3.55 (dd, 1H), 4.92 |
| | (br dd, 1H), 7.36 (t, 1H), 7.40-7.47 (m, 1H), 7.66 (td, 1H), 7.93 (d, 1H), 7.96- |
| | 8.02 (m, 1H), 8.08 (dd, 1H), 8.13 (dt, 1H), 8.31 (dd, 2H). |
| Example 269 | 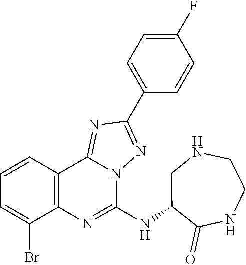 [SMILES: O=C1NCCNC[C@H]1Nc1nc2c(Br)cccc2c2nc(-c3ccc(F)cc3)nn12] |
| | (6R)-6-{[7-bromo-2-(4-fluorophenyl)[1,2,4]triazolo[1,5-c]quinazolin-5- |
| | yl]amino}-1,4-diazepan-5-one |
| | LC-MS (Method 2): Rₜ = 1.20 min; MS (ESIpos): m/z = 470 [M + H]⁺ |
| | ¹H-NMR (400 MHz, DMSO-d₆): δ [ppm] = 2.53-2.58 (m, 1H), 2.69-2.79 (m, |
| | 1H), 3.05 (br dd, 1H), 3.09-3.18 (m, 1H), 3.38-3.48 (m, 1H), 3.55 (dd, 1H), |
| | 4.86-4.94 (m, 1H), 7.35 (t, 1H), 7.43 (t, 2H), 7.88 (d, 1H), 8.08 (dd, 1H), |
| | 8.27-8.36 (m, 4H). |
| Example 270 | 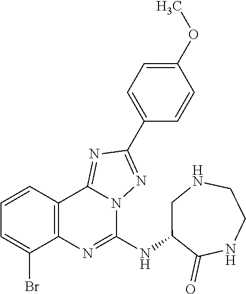 [SMILES: COc1ccc(-c2nc3c4cccc(Br)c4nc(N[C@@H]4CNCCNC4=O)n3n2)cc1] |
| | (6R)-6-{[7-bromo-2-(4-methoxyphenyl)[1,2,4]triazolo[1,5-c]quinazolin-5- |
| | yl]amino}-1,4-diazepan-5-one |
| | LC-MS (Method 2): Rₜ = 1.19 min; MS (ESIneg): m/z = 480 [M − H]⁻ |
| | ¹H-NMR (400 MHz, DMSO-d₆): δ [ppm] = 2.52-2.56 (m, 2H), 2.67-2.77 (m, |
| | 2H), 3.00-3.17 (m, 2H), 3.37-3.48 (m, 1H), 3.54 (br d, 1H), 3.86 (s, 3H), |
| | 4.87-4.93 (m, 1H), 7.15 (d, 2H), 7.35 (t, 1H), 7.84 (d, 1H), 8.07 (dd, 1H), |
| | 8.22 (d, 2H), 8.27-8.34 (m, 2H). |
| Example 271 | 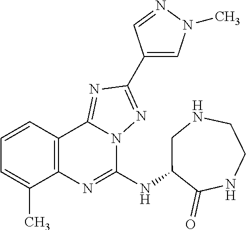 [SMILES: Cc1cccc2c1nc(N[C@@H]1CNCCNC1=O)n1nc(-c3cnn(C)c3)nc21] |
| | (6R)-6-{[7-methyl-2-(1-methyl-1H-pyrazol-4-yl)[1,2,4]triazolo[1,5- |
| | c]quinazolin-5-yl]amino}-1,4-diazepan-5-one |
| | LC-MS (Method 2): Rt = 0.86 min; MS (ESIpos): m/z = 392 [M + H]⁺ |
| Example 272 | 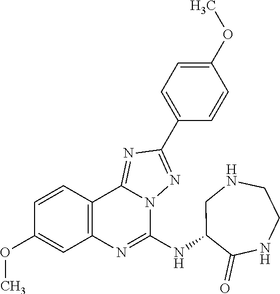 [SMILES: COc1ccc(-c2nc3c4ccc(OC)cc4nc(N[C@@H]4CNCCNC4=O)n3n2)cc1] |
| | (6R)-6-{[8-methoxy-2-(4-methoxyphenyl)[1,2,4]triazolo[1,5-c]quinazolin- |
| | 5-yl]amino}-1,4-diazepan-5-one |
| | LC-MS (Method 2): Rt = 1.05 min; MS (ESIpos): m/z = 434 [M + H]⁺ |
| | ¹H-NMR (400 MHz, DMSO-d₆): δ [ppm] = 2.52-2.55 (m, 1H), 2.65-2.70 (m, |
| | 1H), 3.02 (dd, 1H), 3.06-3.16 (m, 1H), 3.36-3.43 (m, 2H), 3.85 (s, 3H), |
| | 3.92 (s, 3H), 4.87 (br d, 1H), 7.05 (dd, 1H), 7.10-7.15 (m, 3H), 7.63 (br s, |
| | 1H), 8.16-8.22 (m, 3H), 8.30 (dd, 1H). |
| Example 273 | 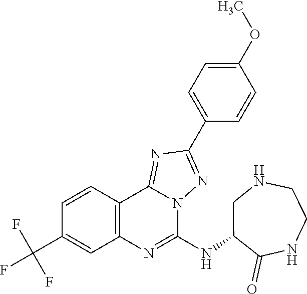 [SMILES: COc1ccc(-c2nc3c4ccc(C(F)(F)F)cc4nc(N[C@@H]4CNCCNC4=O)n3n2)cc1] |
| | (6R)-6-{[2-(4-methoxyphenyl)-8-(trifluoromethyl)[1,2,4]triazolo[1,5- |
| | c]quinazolin-5-yl]amino}-1,4-diazepan-5-one |
| | LC-MS (Method 2): Rt = 1.22 min; MS (ESIpos): m/z = 472 [M + H]⁺ |
| | ¹H-NMR (400 MHz, DMSO-d₆): δ [ppm] = 2.65-2.74 (m, 1H), 2.96 (brs, 1H), |
| | 3.03 (br dd, 1H), 3.08-3.16 (m, 1H), 3.36-3.47 (m, 2H), 3.86 (s, 3H), 4.90 |
| | (br dd, 1H), 7.13-7.18 (m, 2H), 7.72 (dd, 1H), 7.83 (d, 1H), 7.95 (s, 1H), |
| | 8.21-8.26 (m, 2H), 8.33 (dd, 1H), 8.49 (d, 1H). |
| Example 274 | 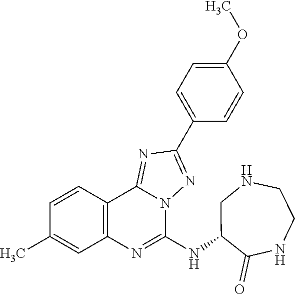 [SMILES: COc1ccc(-c2nc3c4ccc(C)cc4nc(N[C@@H]4CNCCNC4=O)n3n2)cc1] |
| | (6R)-6-{[2-(4-methoxyphenyl)-8-methyl[1,2,4]triazolo[1,5-c]quinazolin-5- |
| | yl]amino}-1,4-diazepan-5-one |
| | LC-MS (Method 2): Rt = 1.10 min; MS (ESIpos): m/z = 418 [M + H]⁺ |
| | ¹H-NMR (400 MHz, DMSO-d₆): δ [ppm] = 2.61-2.72 (m, 1H), 2.90 (br s, 1H), |
| | 3.03 (br d, 1H), 3.07-3.16 (m, 1H), 3.35-3.46 (m, 2H), 3.86 (s, 3H), 4.81- |
| | 4.89 (m, 1H), 7.13 (s, 1H), 7.15 (s, 1H), 7.29 (dd, 1H), 7.50 (s, 1H), 7.62 (d, |
| | 1H), 8.18 (d, 1H), 8.19-8.23 (m, 2H), 8.29 (dd, 1H). |
| Example 275 | 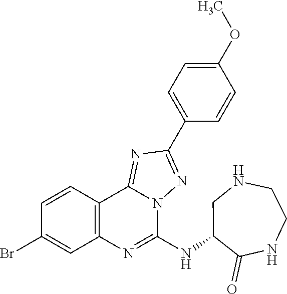 [SMILES: COc1ccc(-c2nc3c4ccc(Br)cc4nc(N[C@@H]4CNCCNC4=O)n3n2)cc1] |
| | (6R)-6-{[8-bromo-2-(4-methoxyphenyl)[1,2,4]triazolo[1,5-c]quinazolin-5- |
| | yl]amino}-1,4-diazepan-5-one |
| | LC-MS (Method 2): Rt = 1.19 min; MS (ESIpos): m/z = 482 [M + H]⁺ |
| | ¹H-NMR (400 MHz, DMSO-d₆): δ [ppm] = 2.62-2.73 (m, 2H), 2.91 (br s, 1H), |
| | 3.02 (br d, 1H), 3.06-3.15 (m, 1H), 3.35-3.46 (m, 2H), 3.85 (s, 3H), 4.86 |
| | (br d, 1H), 7.11-7.17 (m, 2H), 7.59 (dd, 1H), 7.76 (br d, 1H), 7.85 (d, 1H), |
| | 8.18-8.23 (m, 3H), 8.31 (br dd, 1H). |
| Example 276 | 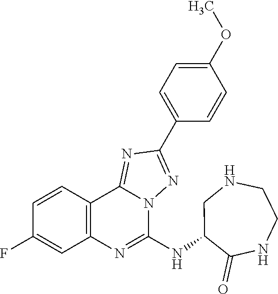 [SMILES: COc1ccc(-c2nc3c4ccc(F)cc4nc(N[C@@H]4CNCCNC4=O)n3n2)cc1] |
| | (6R)-6-{[8-fluoro-2-(4-methoxyphenyl)[1,2,4]triazolo[1,5-c]quinazolin-5- |
| | yl]amino}-1,4-diazepan-5-one |
| | LC-MS (Method 2): Rt = 1.08 min; MS (ESIpos): m/z = 422 [M + H]⁺ |
| | ¹H-NMR (400 MHz, DMSO-d₆): δ [ppm] = 2.65-2.72 (m, 1H), 2.91 (br s, 1H), |
| | 2.99-3.15 (m, 2H), 3.36-3.46 (m, 2H), 3.86 (s, 3H), 4.85-4.90 (m, 1H), |
| | 7.12-7.16 (m, 2H), 7.32 (td, 1H), 7.40 (dd, 1H), 7.77 (d, 1H), 8.19-8.23 (m, |
| | 2H), 8.29-8.36 (m, 2H). |
| Example 277 | 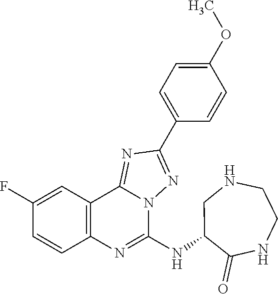 [SMILES: COc1ccc(-c2nc3c4cc(F)ccc4nc(N[C@@H]4CNCCNC4=O)n3n2)cc1] |
| | (6R)-6-{[9-fluoro-2-(4-methoxyphenyl)[1,2,4]triazolo[1,5-c]quinazolin-5- |
| | yl]amino}-1,4-diazepan-5-one |
| | LC-MS (Method 2): Rₜ = 1.09 min; MS (ESIpos): m/z = 422 [M + H]⁺ |
| | ¹H-NMR (400 MHz, DMSO-d₆): δ [ppm] = 2.61-2.73 (m, 1H), 2.88-2.97 (m, |
| | 1H), 3.02 (br dd, 1H), 3.06-3.17 (m, 1H), 3.36-3.50 (m, 2H), 3.82-3.90 |
| | (m, 3H), 4.85 (ddd, 1H), 7.10-7.19 (m, 2H), 7.58-7.67 (m, 2H), 7.69-7.76 |
| | (m, 1H), 8.00 (dd, 1H), 8.17-8.26 (m, 2H), 8.30 (dd, 1H). |
| Example 278 | 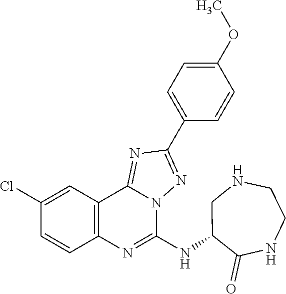 [SMILES: COc1ccc(-c2nc3c4cc(Cl)ccc4nc(N[C@@H]4CNCCNC4=O)n3n2)cc1] |
| | (6R)-6-{[9-chloro-2-(4-methoxyphenyl)[1,2,4]triazolo[1,5-c]quinazolin-5- |
| | yl]amino}-1,4-diazepan-5-one |
| | LC-MS (Method 2): Rₜ = 1.20 min; MS (ESIpos): m/z = 438 [M + H]⁺ |
| | ¹H-NMR (400 MHz, DMSO-d₆): δ [ppm] = 2.70 (d, 1H), 3.02 (br dd, 1H), 3.06- |
| | 3.16 (m, 1H), 3.37-3.47 (m, 3H), 3.85 (s, 3H), 4.86 (ddd, 1H), 7.11-7.16 |
| | (m, 2H), 7.64-7.76 (m, 3H), 8.17-8.23 (m, 3H), 8.31 (dd, 1H). |
| Example 279 | 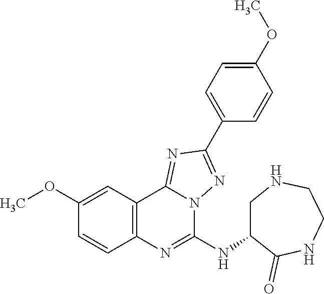 [SMILES: COc1ccc(-c2nc3c4cc(OC)ccc4nc(N[C@@H]4CNCCNC4=O)n3n2)cc1] |
| | (6R)-6-{[9-methoxy-2-(4-methoxyphenyl)[1,2,4]triazolo[1,5-c]quinazolin- |
| | 5-yl]amino}-1,4-diazepan-5-one |
| | LC-MS (Method 2): Rₜ = 1.04 min; MS (ESIpos): m/z = 434 [M + H]⁺ |
| | ¹H-NMR (400 MHz, DMSO-d₆): δ [ppm] = 2.63 (s, 1H), 2.85-2.96 (m, 1H), |
| | 2.98-3.17 (m, 2H), 3.36-3.46 (m, 2H), 3.86 (s, 3H), 3.93 (s, 3H), 4.84 |
| | (ddd, 1H), 7.11-7.19 (m, 2H), 7.35-7.40 (m, 1H), 7.52 (d, 1H), 7.63 (d, |
| | 1H), 7.67 (d, 1H), 8.19-8.25 (m, 2H), 8.28 (dd, 1H). |
| Example 280 | 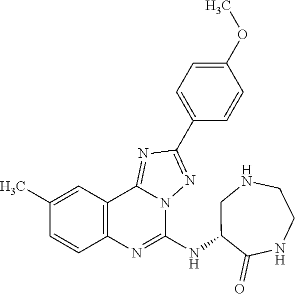 [SMILES: COc1ccc(-c2nc3c4cc(C)ccc4nc(N[C@@H]4CNCCNC4=O)n3n2)cc1] |
| | (6R)-6-{[2-(4-methoxyphenyl)-9-methyl[1,2,4]triazolo[1,5-c]quinazolin-5- |
| | yl]amino}-1,4-diazepan-5-one |
| | LC-MS (Method 2): Rₜ = 1.13 min; MS (ESIpos): m/z = 418 [M + H]⁺ |
| | ¹H-NMR (400 MHz, DMSO-d₆): δ [ppm] = 2.62-2.71 (m, 1H), 2.93 (ddd, 1H), |
| | 3.02 (br dd, 1H), 3.06-3.15 (m, 1H), 3.37-3.47 (m, 2H), 3.85 (s, 3H), 4.85 |
| | (ddd, 1H), 7.10-7.17 (m, 2H), 7.52-7.63 (m, 3H), 8.07-8.11 (m, 1H), 8.17- |
| | 8.25 (m, 2H), 8.28 (dd, 1H). |
| Example 281 | 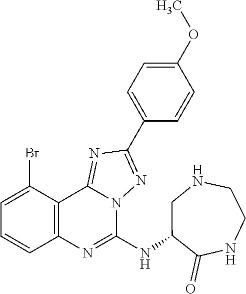 [SMILES: COc1ccc(-c2nc3c4c(Br)cccc4nc(N[C@@H]4CNCCNC4=O)n3n2)cc1] |
| | 6-{[10-bromo-2-(4-methoxyphenyl)[1,2,4]triazolo[1,5-c]quinazolin-5- |
| | yl]amino}-1,4-diazepan-5-one |
| | LC-MS (Method 2): Rₜ = 1.21 min; MS (ESIpos): m/z = 482 [M + H]⁺ |
| | ¹H-NMR (400 MHz, DMSO-d₆): δ [ppm] = 2.64-2.72 (m, 1H), 3.02 (br dd, |
| | 1H), 3.07-3.15 (m, 1H), 3.37-3.47 (m, 2H), 3.86 (s, 3H), 4.88 (ddd, 1H), |
| | 7.13-7.19 (m, 2H), 7.56-7.62 (m, 1H), 7.65-7.69 (m, 1H), 7.72 (dd, 1H), |
| | 7.78 (d, 1H), 8.21-8.26 (m, 2H), 8.31 (dd, 1H). |
| Example 282 | 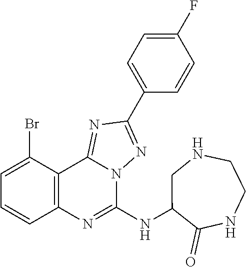 [SMILES: O=C1NCCNCC1Nc1nc2cccc(Br)c2c2nc(-c3ccc(F)cc3)nn12] |
| | 6-{[10-bromo-2-(4-fluorophenyl)[1,2,4]triazolo[1,5-c]quinazolin-5- |
| | yl]amino}-1,4-diazepan-5-one |
| | LC-MS (Method 2): Rₜ = 1.25 min; MS (ESIpos): m/z = 470 [M + H]⁺ |
| | ¹H-NMR (400 MHz, DMSO-d₆): δ [ppm] = 2.64-2.73 (m, 1H), 2.93 (brs, 1H), |
| | 3.02 (br dd, 1H), 3.07-3.16 (m, 1H), 3.36-3.47 (m, 2H), 4.88 (br d, 1H), |
| | 7.41-7.50 (m, 2H), 7.57-7.64 (m, 1H), 7.66-7.70 (m, 1H), 7.73 (dd, 1H), |
| | 7.81 (br d, 1H), 8.27-8.38 (m, 3H). |
| Example 283 | 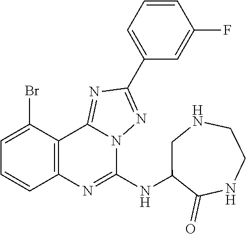 [SMILES: O=C1NCCNCC1Nc1nc2cccc(Br)c2c2nc(-c3cccc(F)c3)nn12] |
| | 6-{[10-bromo-2-(3-fluorophenyl)[1,2,4]triazolo[1,5-c]quinazolin-5- |
| | yl]amino}-1,4-diazepan-5-one |
| | LC-MS (Method 2): Rₜ = 1.25 min; MS (ESIpos): m/z = 470 [M + H]⁺ |
| | 1H-NMR (400 MHz, DMSO-d₆): δ [ppm] = 2.52-2.56 (m, 1H), 2.70 (dd, 1H), |
| | 3.03 (br dd, 1H), 3.07-3.16 (m, 1H), 3.40 (br dd, 2H), 4.89 (ddd, 1H), 7.40- |
| | 7.47 (m, 1H), 7.57-7.65 (m, 1H), 7.65-7.70 (m, 2H), 7.72 (dd, 1H), 7.83 (d, |
| | 1H), 7.96-8.03 (m, 1H), 8.14 (dt, 1H), 8.31 (dd, 1H). |
| Example 284 | 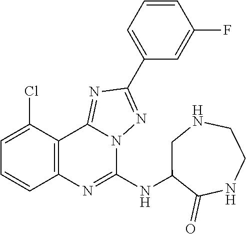 [SMILES: O=C1NCCNCC1Nc1nc2cccc(Cl)c2c2nc(-c3cccc(F)c3)nn12] |
| | 6-{[10-chloro-2-(3-fluorophenyl)[1,2,4]triazolo[1,5-c]quinazolin-5- |
| | yl]amino}-1,4-diazepan-5-one |
| | LC-MS (Method 2): Rₜ = 1.20 min; MS (ESIpos): m/z = 426 [M + H]⁺ |
| | ¹H-NMR (400 MHz, DMSO-d₆): δ [ppm] = 2.64-2.74 (m, 1H), 2.92 (brs, 1H), |
| | 2.98-3.06 (m, 1H), 3.07-3.16 (m, 1H), 3.37-3.48 (m, 2H), 4.89 (ddd, 1H), |
| | 7.40-7.47 (m, 1H), 7.55 (dd, 1H), 7.62-7.73 (m, 3H), 7.85 (d, 1H), 7.97- |
| | 8.03 (m, 1H), 8.14 (dt, 1H), 8.31 (dd, 1H). |
| Example 285 | 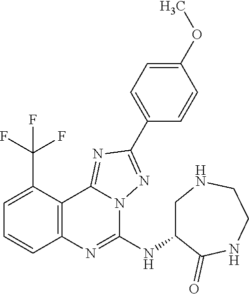 [SMILES: COc1ccc(-c2nc3c4c(C(F)(F)F)cccc4nc(N[C@@H]4CNCCNC4=O)n3n2)cc1] |
| | (6R)-6-{[2-(4-methoxyphenyl)-10-(trifluoromethyl)[1,2,4]triazolo[1,5- |
| | c]quinazolin-5-yl]amino}-1,4-diazepan-5-one |
| | LC-MS (Method 2): Rₜ = 1.24 min; MS (ESIpos): m/z = 472 [M + H]⁺ |
| | ¹H-NMR (400 MHz, DMSO-d₆): δ [ppm] = 2.65-2.74 (m, 1H), 2.99-3.06 (m, |
| | 1H), 3.08-3.17 (m, 1H), 3.38-3.49 (m, 2H), 3.86 (s, 3H), 4.90 (ddd, 1H), |
| | 7.14-7.20 (m, 2H), 7.82-7.90 (m, 3H), 7.92-7.99 (m, 1H), 8.18-8.24 (m, |
| | 2H), 8.31 (dd, 1H). |
| Example 286 | 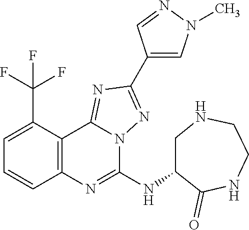 [SMILES: Cn1cc(-c2nc3c4c(C(F)(F)F)cccc4nc(N[C@@H]4CNCCNC4=O)n3n2)cn1] |
| | (6R)-6-{[2-(1-methyl-1H-pyrazol-4-yl)-10- |
| | (trifluoromethyl)[1,2,4]triazolo[1,5-c]quinazolin-5-yl]amino}-1,4- |
| | diazepan-5-one |
| | LC-MS (Method 2): Rₜ = 0.97 min; MS (ESIpos): m/z = 446 [M + H]⁺ |
| | ¹H-NMR (400 MHz, DMSO-d₆): δ [ppm] = 2.67 (dd, 1H), 3.02 (br dd, 1H), 3.07- |
| | 3.16 (m, 1H), 3.96 (s, 3H), 4.89 (br dd, 1H), 7.73 (d, 1H), 7.83-7.87 (m, |
| | 2H), 7.92-7.97 (m, 1H), 8.03 (s, 1H), 8.31 (dd, 1H), 8.43 (s, 1H). |
| Example 287 | 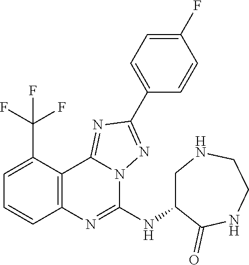 [SMILES: O=C1NCCNC[C@H]1Nc1nc2cccc(C(F)(F)F)c2c2nc(-c3ccc(F)cc3)nn12] |
| | (6R)-6-{[2-(4-fluorophenyl)-10-(trifluoromethyl)[1,2,4]triazolo[1,5- |
| | c]quinazolin-5-yl]amino}-1,4-diazepan-5-one |
| | LC-MS (Method 2): Rₜ = 1.27 min; MS (ESIpos): m/z = 460 [M + H]⁺ |
| | ¹H-NMR (400 MHz, DMSO-d₆): δ [ppm] = 2.66-2.74 (m, 1H), 2.94 (brs, 1H), |
| | 3.03 (br dd, 1H), 3.08-3.17 (m, 1H), 3.39-3.48 (m, 2H), 4.86-4.94 (m, |
| | 1H), 7.42-7.50 (m, 2H), 7.85-7.90 (m, 3H), 7.93-8.00 (m, 1H), 8.28-8.35 |
| | (m, 3H). |
| Example 288 | 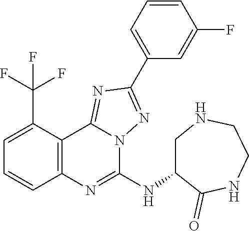 [SMILES: O=C1NCCNC[C@H]1Nc1nc2cccc(C(F)(F)F)c2c2nc(-c3cccc(F)c3)nn12] |
| | (6R)-6-{[2-(3-fluorophenyl)-10-(trifluoromethyl)[1,2,4]triazolo[1,5- |
| | c]quinazolin-5-yl]amino}-1,4-diazepan-5-one |
| | LC-MS (Method 2): Rₜ = 1.27 min; MS (ESIpos): m/z = 460 [M + H]⁺ |
| | ¹H-NMR (400 MHz, DMSO-d₆): δ [ppm] = 2.72 (dd, 1H), 3.03 (br dd, 1H), 3.08- |
| | 3.17 (m, 1H), 3.38-3.49 (m, 2H), 4.91 (ddd, 1H), 7.41-7.48 (m, 1H), 7.68 |
| | (td, 1H), 7.86-7.93 (m, 3H), 7.94-8.00 (m, 2H), 8.13 (dt, 1H), 8.32 (dd, |
| | 1H). |
| Example 289 | 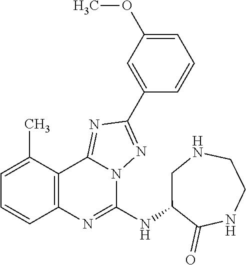 [SMILES: COc1cccc(-c2nc3c4c(C)cccc4nc(N[C@@H]4CNCCNC4=O)n3n2)c1] |
| | (6R)-6-{[2-(3-methoxyphenyl)-10-methyl[1,2,4]triazolo[1,5-c]quinazolin- |
| | 5-yl]amino}-1,4-diazepan-5-one |
| | LC-MS (Method 2): Rₜ = 1.24 min; MS (ESIpos): m/z = 418 [M + H]⁺ |
| | ¹H-NMR (400 MHz, DMSO-d₆): δ [ppm] = 2.65-2.74 (m, 1H), 2.99-3.06 (m, |
| | 4H), 3.07-3.17 (m, 1H), 3.38-3.48 (m, 3H), 3.88 (s, 3H), 4.88 (ddd, 1H), |
| | 7.15 (ddd, 1H), 7.28 (d, 1H), 7.49-7.55 (m, 2H), 7.58-7.64 (m, 1H), 7.68 |
| | (d, 1H), 7.79 (dd, 1H), 7.88 (dt, 1H), 8.28 (dd, 1H). |
| Example 290 | 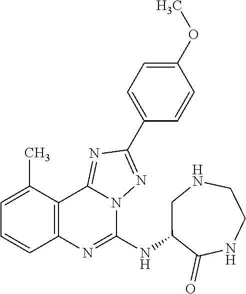 [SMILES: COc1ccc(-c2nc3c4c(C)cccc4nc(N[C@@H]4CNCCNC4=O)n3n2)cc1] |
| | (6R)-6-{[2-(4-methoxyphenyl)-10-methyl[1,2,4]triazolo[1,5-c]quinazolin- |
| | 5-yl]amino}-1,4-diazepan-5-one |
| | LC-MS (Method 2): Rₜ = 1.22 min; MS (ESIpos): m/z = 419 [M + H]⁺ |
| | ¹H-NMR (400 MHz, DMSO-d₆): δ [ppm] = 2.53 (d, 1H), 2.61-2.71 (m, 1H), |
| | 2.99-3.05 (m, 4H), 3.07-3.16 (m, 1H), 3.37-3.48 (m, 2H), 3.86 (s, 3H), |
| | 4.87 (ddd, 1H), 7.12-7.18 (m, 2H), 7.27 (d, 1H), 7.49-7.54 (m, 1H), 7.57- |
| | 7.66 (m, 2H), 8.20-8.25 (m, 2H), 8.29 (dd, 1H). |
| Example 291 | 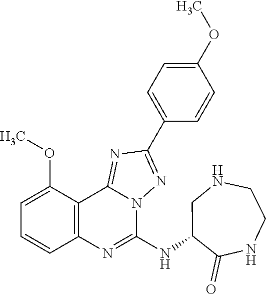 [SMILES: COc1ccc(-c2nc3c4c(OC)cccc4nc(N[C@@H]4CNCCNC4=O)n3n2)cc1] |
| | (6R)-6-{[10-methoxy-2-(4-methoxyphenyl)[1,2,4]triazolo[1,5- |
| | c]quinazolin-5-yl]amino}-1,4-diazepan-5-one |
| | LC-MS (Method 2): Rₜ = 0.95 min; MS (ESIneg): m/z = 432 [M − H]⁻ |
| Example 292 | 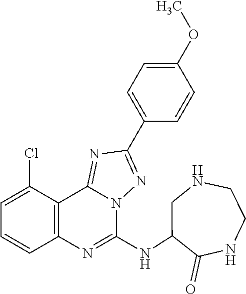 [SMILES: COc1ccc(-c2nc3c4c(Cl)cccc4nc(NC4CNCCNC4=O)n3n2)cc1] |
| | 6-{[10-chloro-2-(4-methoxyphenyl)[1,2,4]triazolo[1,5-c]quinazolin-5- |
| | yl]amino}-1,4-diazepan-5-one |
| | LC-MS (Method 2): Rₜ = 1.19 min; MS (ESIpos): m/z = 438 [M + H]⁺ |
| | ¹H-NMR (400 MHz, DMSO-d₆): δ [ppm] = 2.65-2.74 (m, 1H), 3.04 (br dd, |
| | 1H), 3.08-3.17 (m, 1H), 3.38-3.49 (m, 2H), 3.86 (s, 3H), 4.84-4.92 (m, |
| | 1H), 7.13-7.19 (m, 2H), 7.53 (dd, 1H), 7.61-7.71 (m, 2H), 7.80 (d, 1H), |
| | 8.20-8.26 (m, 2H), 8.32 (dd, 1H). |
| Example 293 | 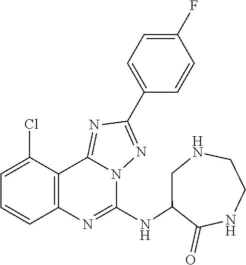 [SMILES: O=C1NCCNCC1Nc1nc2cccc(Cl)c2c2nc(-c3ccc(F)cc3)nn12] |
| | 6-{[10-chloro-2-(4-fluorophenyl)[1,2,4]triazolo[1,5-c]quinazolin-5- |
| | yl]amino}-1,4-diazepan-5-one |
| | LC-MS (Method 2): Rₜ = 1.21 min; MS (ESIpos): m/z = 426 [M + H]⁺ |
| | ¹H-NMR (400 MHz, DMSO-d₆): δ [ppm] = 2.65-2.72 (m, 1H), 3.07-3.16 (m, |
| | 1H), 3.37-3.48 (m, 2H), 4.88 (ddd, 1H), 7.41-7.49 (m, 2H), 7.54 (dd, 1H), |
| | 7.62-7.72 (m, 2H), 7.82 (d, 1H), 8.28-8.38 (m, 3H). |
| Example 294 | 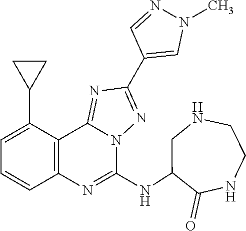 [SMILES: Cn1cc(-c2nc3c4c(C5CC5)cccc4nc(NC4CNCCNC4=O)n3n2)cn1] |
| | 6-{[10-cyclopropyl-2-(1-methyl-1H-pyrazol-4-yl)[1,2,4]triazolo[1,5- |
| | c]quinazolin-5-yl]amino}-1,4-diazepan-5-one |
| | LC-MS (Method 2): Rₜ = 1.00 min; MS (ESIpos): m/z = 418 [M + H]⁺ |
| | ¹H-NMR (400 MHz, DMSO-d₆): δ [ppm] = 0.82-0.88 (m, 2H), 1.15-1.22 (m, |
| | 2H), 2.60-2.66 (m, 1H), 2.89 (dt, 1H), 3.02 (br dd, 1H), 3.06-3.16 (m, 1H), |
| | 3.36-3.47 (m, 2H), 3.80 (tt, 1H), 3.95 (s, 3H), 4.82-4.89 (m, 1H), 6.95 (dd, |
| | 1H), 7.46 (dd, 1H), 7.52-7.61 (m, 2H), 8.05 (d, 1H), 8.29 (dd, 1H), 8.47 (s, |
| | 1H). |
| Example 295 | 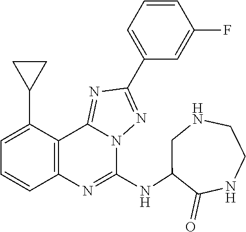 [SMILES: O=C1NCCNCC1Nc1nc2cccc(C3CC3)c2c2nc(-c3cccc(F)c3)nn12] |
| | 6-{[10-cyclopropyl-2-(3-fluorophenyl)[1,2,4]triazolo[1,5-c]quinazolin-5- |
| | yl]amino}-1,4-diazepan-5-one |
| | LC-MS (Method 2): Rₜ = 1.34 min; MS (ESIpos): m/z = 432 [M + H]⁺ |
| | ¹H-NMR (400 MHz, DMSO-d₆): δ [ppm] = 0.84-0.90 (m, 2H), 1.19-1.27 (m, |
| | 2H), 2.68-2.73 (m, 1H), 2.86-2.98 (m, 1H), 3.02 (br dd, 1H), 3.07-3.16 |
| | (m, 1H), 3.38-3.48 (m, 2H), 3.80 (tt, 1H), 4.88 (ddd, 1H), 6.99 (d, 1H), 7.39- |
| | 7.46 (m, 1H), 7.49 (dd, 1H), 7.58-7.69 (m, 2H), 7.71 (d, 1H), 8.01 (ddd, |
| | 1H), 8.14 (dt, 1H), 8.30 (dd, 1H). |

The following compounds were synthesised analogously to example 192:

| | Structure |
|---|---|
| | IUPAC-Name |
| | LC-MS (method): Retention time; Mass found |
| Example | ¹H-NMR |
| Example 292 | 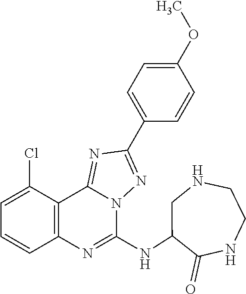 [SMILES: COc1ccc(-c2nc3c4c(Cl)cccc4nc(NC4CNCCNC4=O)n3n2)cc1] |
| | 6-{[10-chloro-2-(4-methoxyphenyl)[1,2,4]triazolo[1,5-c]quinazolin-5- |
| | yl]amino}-1,4-diazepan-5-one |
| | LC-MS (Method 2): Rₜ = 1.19 min; MS (ESIpos): m/z = 438 [M + H]⁺ |
| | ¹H-NMR (400 MHz, DMSO-d₆): δ [ppm] = 2.65-2.74 (m, 1H), 3.04 (br dd, |
| | 1H), 3.08-3.17 (m, 1H), 3.38-3.49 (m, 2H), 3.86 (s, 3H), 4.84-4.92 (m, |
| | 1H), 7.13-7.19 (m, 2H), 7.53 (dd, 1H), 7.61-7.71 (m, 2H), 7.80 (d, 1H), |
| | 8.20-8.26 (m, 2H), 8.32 (dd, 1H). |
| Example 293 | 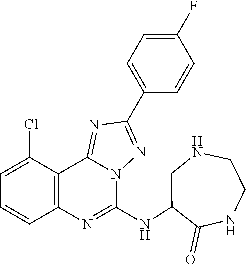 [SMILES: O=C1NCCNCC1Nc1nc2cccc(Cl)c2c2nc(-c3ccc(F)cc3)nn12] |
| | 6-{[10-chloro-2-(4-fluorophenyl)[1,2,4]triazolo[1,5-c]quinazolin-5- |
| | yl]amino}-1,4-diazepan-5-one |
| | LC-MS (Method 2): Rₜ = 1.21 min; MS (ESIpos): m/z = 426 [M + H]⁺ |
| | ¹H-NMR (400 MHz, DMSO-d₆): δ [ppm] = 2.65-2.72 (m, 1H), 3.07-3.16 (m, |
| | 1H), 3.37-3.48 (m, 2H), 4.88 (ddd, 1H), 7.41-7.49 (m, 2H), 7.54 (dd, 1H), |
| | 7.62-7.72 (m, 2H), 7.82 (d, 1H), 8.28-8.38 (m, 3H). |
| Example 294 | 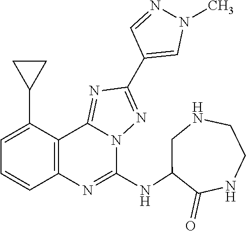 [SMILES: Cn1cc(-c2nc3c4c(C5CC5)cccc4nc(NC4CNCCNC4=O)n3n2)cn1] |
| | 6-{[10-cyclopropyl-2-(1-methyl-1H-pyrazol-4-yl)[1,2,4]triazolo[1,5- |
| | c]quinazolin-5-yl]amino}-1,4-diazepan-5-one |
| | LC-MS (Method 2): Rₜ = 1.00 min; MS (ESIpos): m/z = 418 [M + H]⁺ |
| | ¹H-NMR (400 MHz, DMSO-d₆): δ [ppm] = 0.82-0.88 (m, 2H), 1.15-1.22 (m, |
| | 2H), 2.60-2.66 (m, 1H), 2.89 (dt, 1H), 3.02 (br dd, 1H), 3.06-3.16 (m, 1H), |
| | 3.36-3.47 (m, 2H), 3.80 (tt, 1H), 3.95 (s, 3H), 4.82-4.89 (m, 1H), 6.95 (dd, |
| | 1H), 7.46 (dd, 1H), 7.52-7.61 (m, 2H), 8.05 (d, 1H), 8.29 (dd, 1H), 8.47 (s, |
| | 1H). |
| Example 295 | 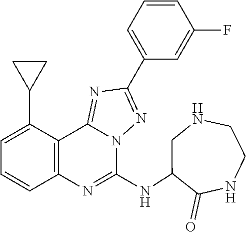 [SMILES: O=C1NCCNCC1Nc1nc2cccc(C3CC3)c2c2nc(-c3cccc(F)c3)nn12] |
| | 6-{[10-cyclopropyl-2-(3-fluorophenyl)[1,2,4]triazolo[1,5-c]quinazolin-5- |
| | yl]amino}-1,4-diazepan-5-one |
| | LC-MS (Method 2): Rₜ = 1.34 min; MS (ESIpos): m/z = 432 [M + H]⁺ |
| | ¹H-NMR (400 MHz, DMSO-d₆): δ [ppm] = 0.84-0.90 (m, 2H), 1.19-1.27 (m, |
| | 2H), 2.68-2.73 (m, 1H), 2.86-2.98 (m, 1H), 3.02 (br dd, 1H), 3.07-3.16 |
| | (m, 1H), 3.38-3.48 (m, 2H), 3.80 (tt, 1H), 4.88 (ddd, 1H), 6.99 (d, 1H), 7.39- |
| | 7.46 (m, 1H), 7.49 (dd, 1H), 7.58-7.69 (m, 2H), 7.71 (d, 1H), 8.01 (ddd, |
| | 1H), 8.14 (dt, 1H), 8.30 (dd, 1H). |

### Example 296

### 6-{[10-Chloro-2-(4-methoxyphenyl)[1,2,4]triazolo[1,5-c]quinazolin-5-yl]amino}-1,4-diazepan-5-one, Enantiomer 1

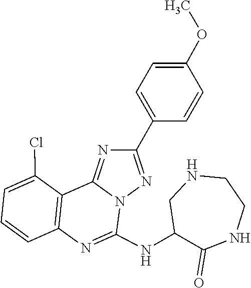 [SMILES: COc1ccc(-c2nc3c4c(Cl)cccc4nc(NC4CNCCNC4=O)n3n2)cc1]

Chiral HPLC separation of example 292 was performed (Instrument: PrepCon Labomatic HPLC; Column: YMC Cellulose SB 5μ 250×30; Eluent A: Hexane+0.1% diethylamine; Eluent B: 2-Propanol; Gradient: 30->50% B in 15 min; Flow: 40 mL/min; Temperature: 25° C.; UV: 254 nm).

Retention time of enantiomer 1: 4.09 min; [α]²⁰_{D}: −55° (c=1) in DMSO

Instrument: Agilent 1260 HPLC; Column: YMC Cellulose SB 3μ 100×4.6; Eluent A: Hexane+0.1% diethylamine; Eluent B: 2-Propanol; Gradient: 20->50% B in 7 min; Flow: Temperature: 60° C.; UV: 254 nm.

### Example 297

### 6-{[10-chloro-2-(4-methoxyphenyl)[1,2,4]triazolo[1,5-c]quinazolin-5-yl]amino}-1,4-diazepan-5-one, Enantiomer 2

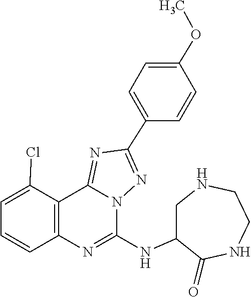 [SMILES: COc1ccc(-c2nc3c4c(Cl)cccc4nc(NC4CNCCNC4=O)n3n2)cc1]

The title compound was prepared as described for example 296

Retention time of enantiomer 2: 5.00 min; [α]²⁰_{D}: +75° (c=1) in DMSO

### Example 298

### 6-{[10-chloro-2-(4-fluorophenyl)[1,2,4]triazolo[1,5-c]quinazolin-5-yl]amino}-1,4-diazepan-5-one, Enantiomer 1

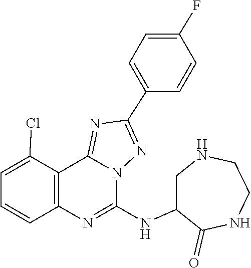 [SMILES: O=C1NCCNCC1Nc1nc2cccc(Cl)c2c2nc(-c3ccc(F)cc3)nn12]

Chiral HPLC separation of example 293 was performed (Instrument: PrepCon Labomatic HPLC; Column: Chiralpak IA 5μ 250×30; Eluent A: Hexane+0.1% diethylamine; Eluent B: 2-Propanol; Gradient: 20->50% B in 10 min; Flow: 40 mL/min; Temperature: 25° C.; UV: 254 nm).

Retention time of enantiomer 1: 3.06 min; [α]²⁰_{D}: −55° (c=1) in DMSO.

Instrument: Agilent 1260 HPLC; Column: Chiralpak IA 3μ 100×4.6; Eluent A: Hexane+0.1% diethylamine; Eluent B: 2-Propanol; Gradient: 20->50% B in 7 min; Flow: Temperature: 60° C.; UV: 254 nm;

### Example 299

### 6-{[10-chloro-2-(4-fluorophenyl)[1,2,4]triazolo[1,5-c]quinazolin-5-yl]amino}-1,4-diazepan-5-one, Enantiomer 2

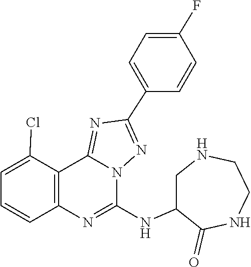 [SMILES: O=C1NCCNCC1Nc1nc2cccc(Cl)c2c2nc(-c3ccc(F)cc3)nn12]

The title compound was prepared as described for example 298

Retention time of enantiomer 2: 4.04 min; [α]₂₀^{D}: +69° (c=1) in DMSO

### Example 300

### 6-{[10-cyclopropyl-2-(3-fluorophenyl)[1,2,4]triazolo[1,5-c]quinazolin-5-yl]amino}-1,4-diazepan-5-one, Enantiomer 1

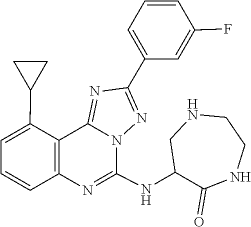 [SMILES: O=C1NCCNCC1Nc1nc2cccc(C3CC3)c2c2nc(-c3cccc(F)c3)nn12]

Chiral HPLC separation of example 295 was performed (Instrument: Sepiatec: Prep SFC100; Column: Chiralpak IG 5μ 250×30 mm; Eluent A: CO2; Eluent B: 2-Propanol+0.4% Diethylamine (99%); Isocratic: 35% B; Flow: 100 mL/min; Temperature: 40° C.; BPR: 150 bar; UV: 220 nm;

Retention time of enantiomer 1: 1.79 min; [α]20 D: −64° (c=1) in DMSO

Instrument: Agilent: 1260, Aurora SFC-Modul; Column: Chiralpak IG 5μ 100×4.6 mm; Eluent A: CO2; Eluent B: 2-Propanol+0.2% Diethylamine (99%); isocratic: 35% B; Flow: 4 mL/min; Temperature: 37.5° C.; BPR: 100 bar; UV: 220 nm;

### Example 301

### 6-{[10-cyclopropyl-2-(3-fluorophenyl)[1,2,4]triazolo[1,5-c]quinazolin-5-yl]amino}-1,4-diazepan-5-one, Enantiomer 2

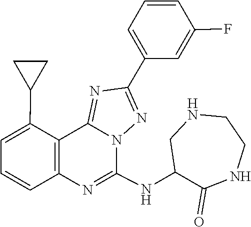 [SMILES: O=C1NCCNCC1Nc1nc2cccc(C3CC3)c2c2nc(-c3cccc(F)c3)nn12]

The title compound was prepared as described for example 300

Retention time of enantiomer 2: 3.63 min; [α]₂₀^{D}: +73° (c=1) in DMSO

### Example 302

### (3R)-3-({2-[3-(Hydroxymethyl)-1,2,4-oxadiazol-5-yl][1,2,4]triazolo[1,5-c]quinazolin-5-yl}amino)azepan-2-one

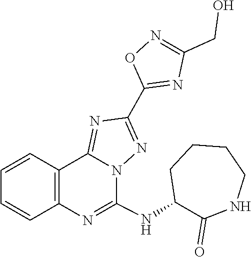 [SMILES: O=C1NCCCC[C@H]1Nc1nc2ccccc2c2nc(-c3nc(CO)no3)nn12]

Ethyl 5-{[(3R)-2-oxoazepan-3-yl]amino}[1,2,4]triazolo[1,5-c]quinazoline-2-carboxylate (100 mg, 271 μmol), N-dihydroxyethanimidamide (55.0 mg, 611 μmol) and cesium carbonate (88.4 mg, 271 μmol) were stirred in 1,4-dioxane (2.5 mL, 29 mmol) for 5 h at 110° C. The reaction mixture was diluted with water and extracted with ethyl acetate. The organic phase was dried over sodium sulphate, filtered and concentrated under reduced pressure. The crude material was purified by preparative HPLC to give 1.00 mg (95% purity, 1% yield) of the title compound.

LC-MS (Method 2): Rₜ=0.96 min; MS (ESIpos): m/z=395 [M+H]⁺

¹H-NMR (400 MHz, DMSO-d₆): δ [ppm]=1.27-1.39 (m, 1H), 1.50-1.62 (m, 1H), 1.81-1.95 (m, 2H), 1.98-2.07 (m, 1H), 2.33-2.38 (m, 1H), 3.12-3.30 (m, 1H), 3.36-3.42 (m, 1H), 4.73 (d, 2H), 4.83 (br dd, 1H), 5.93 (t, 1H), 7.52 (t, 1H), 7.70-7.76 (m, 1H), 7.78-7.89 (m, 2H), 8.27 (t, 1H), 8.34 (d, 1H).

### Example 303

### (3R)-3-({2-[3-(Methoxymethyl)-1,2,4-oxadiazol-5-yl][1,2,4]triazolo[1,5-c]quinazolin-5-yl}amino)azepan-2-one

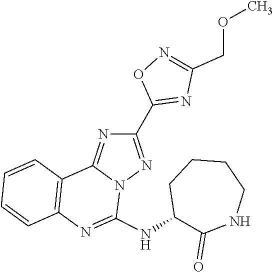 [SMILES: COCc1noc(-c2nc3c4ccccc4nc(N[C@@H]4CCCCNC4=O)n3n2)n1]

Ethyl 5-{[(3R)-2-oxoazepan-3-yl]amino}[1,2,4]triazolo[1,5-c]quinazoline-2-carboxylate (100 mg, 271 μmol), N-hydroxy(methoxy)ethanimidamide (63.6 mg, 611 μmol) and cesium carbonate (88.4 mg, 271 μmol) were stirred in 1,4-dioxane (2.5 mL, 29 mmol) for 5 h at 110° C. The reaction mixture was diluted with water. The solid was filtered and washed once with 10% aqeuous sodium hydrogen carbonate solution and water. The solid was dried under reduced pressure at 60° C. to give 45.0 mg (95% purity, 39% yield) of the title compound without further purification.

LC-MS (method 2): Rₜ=1.11 min; MS (ESIneg): m/z=407 [M−H]⁻.

¹H-NMR (400 MHz, DMSO-d₆): δ [ppm]=1.26-1.42 (m, 1H), 1.50-1.64 (m, 1H), 1.80-1.93 (m, 2H), 1.98-2.08 (m, 1H), 2.34 (br d, 1H), 3.11-3.22 (m, 1H), 3.35-3.41 (m, 1H), 3.43 (s, 3H), 4.74 (s, 2H), 4.83 (br dd, 1H), 7.52 (ddd, 1H), 7.66-7.75 (m, 1H), 7.77-7.84 (m, 1H), 7.88 (d, 1H), 8.26 (dd, 1H), 8.34 (dd, 1H).

### Example 304

### (3R)-3-[(2-{3-[(Dimethylamino)methyl]-1,2,4-oxadiazol-5-yl}[1,2,4]triazolo[1,5-c]quinazolin-5-yl)amino]azepan-2-one

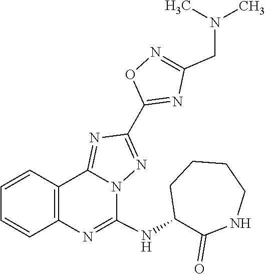 [SMILES: CN(C)Cc1noc(-c2nc3c4ccccc4nc(N[C@@H]4CCCCNC4=O)n3n2)n1]

Ethyl 5-{[(3R)-2-oxoazepan-3-yl]amino}[1,2,4]triazolo[1,5-c]quinazoline-2-carboxylate (100 mg, 271 μmol), (dimethylamino)-N-hydroxyethanimidamide (71.6 mg, 611 μmol) and cesium carbonate (88.4 mg, 271 μmol) were stirred in 1,4-dioxane (2.5 mL, 29 mmol) for 5 h at 110° C. The reaction mixture was diluted with water. The suspension was filtered, the solid was washed with water, and dried under reduced pressure to give 27.0 mg (96% purity, 23% yield) of the title compound without further purification.

LC-MS (method 2): Rₜ=1.09 min; MS (ESIneg): m/z=420 [M−H]⁻.

¹H-NMR (400 MHz, DMSO-d₆): δ [ppm]=1.27-1.41 (m, 1H), 1.46-1.68 (m, 1H), 1.80-1.92 (m, 2H), 1.96-2.08 (m, 1H), 2.24-2.40 (m, 7H), 3.09-3.23 (m, 1H), 3.35-3.43 (m, 1H), 3.56 (s, 1H), 3.79 (s, 2H), 4.84 (br dd, 1H), 7.52 (td, 1H), 7.69-7.75 (m, 1H), 7.78-7.84 (m, 1H), 7.87 (d, 1H), 8.26 (dd, 1H), 8.34 (dd, 1H).

### Example 305

### (3R)-3-({2-[3-(2-Hydroxyethyl)-1,2,4-oxadiazol-5-yl][1,2,4]triazolo[1,5-c]quinazolin-5-yl}amino)azepan-2-one

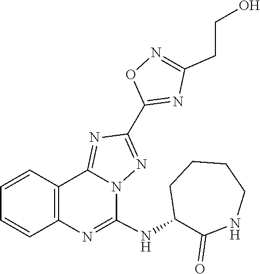 [SMILES: O=C1NCCCC[C@H]1Nc1nc2ccccc2c2nc(-c3nc(CCO)no3)nn12]

Ethyl 5-{[(3R)-2-oxoazepan-3-yl]amino}[1,2,4]triazolo[1,5-c]quinazoline-2-carboxylate (70.0 mg, 190 μmol), N,3-dihydroxypropanimidamide (44.5 mg, 428 μmol) and cesium carbonate (61.9 mg, 190 μmol) were stirred in 1,4-dioxane (2.5 mL, 29 mmol) for 5 h at 110° C. The reaction mixture was diluted with water. The suspension was filtered, the solid was washed with water and dried under reduced pressure to give 9.80 mg (96% purity, 12% yield) of the title compound without further purification.

LC-MS (method 2): Rₜ=0.99 min; MS (ESIpos): m/z=409 [M+H]⁺

¹H-NMR (400 MHz, DMSO-d₆): δ [ppm]=1.17-1.40 (m, 1H), 1.50-1.64 (m, 1H), 1.81-1.94 (m, 2H), 1.98-2.08 (m, 1H), 2.30-2.37 (m, 1H), 3.02 (t, 2H), 3.12-3.21 (m, 1H), 3.35-3.42 (m, 1H), 3.88 (q, 2H), 4.84 (br dd, 1H), 4.92 (t, 1H), 7.51 (ddd, 1H), 7.69-7.75 (m, 1H), 7.78-7.83 (m, 1H), 7.87 (d, 1H), 8.26 (dd, 1H), 8.34 (dd, 1H).

### Example 306

### (3R)-3-({2-[4-(S-methylsulfonimidoyl)phenyl][1,2,4]triazolo[1,5-c]quinazolin-5-yl}amino)azepan-2-one

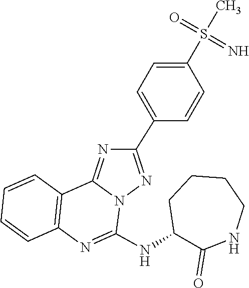 [SMILES: CS(=N)(=O)c1ccc(-c2nc3c4ccccc4nc(N[C@@H]4CCCCNC4=O)n3n2)cc1]

(3R)-3-({2-[4-(Methylsulfanyl)phenyl][1,2,4]triazolo[1,5-c]quinazolin-5-yl}amino)azepan-2-one (50.0 mg, 119 μmol), (diacetoxyiodo)benzene (96.2 mg, 299 μmol) and ammonium carbamate (18.7 mg, 239 μmol) were stirred in methanol (240 μL) for 2 h at rt under argon. The reaction mixture was concentrated under reduced pressure and purified by preparative HPLC to give 10.7 mg (100% purity, 20% yield) of the title compound.

LC-MS (method 2): Rₜ=1.06 min; MS (ESIneg): m/z=448 [M−H]⁻.

¹H-NMR (400 MHz, DMSO-d₆): δ [ppm]=1.27-1.39 (m, 1H), 1.52-1.64 (m, 1H), 1.81-1.96 (m, 2H), 1.99-2.08 (m, 1H), 2.29-2.38 (m, 1H), 3.15 (d, 3H), 3.16-3.22 (m, 1H), 3.34-3.42 (m, 1H), 4.39 (d, 1H), 4.85 (br dd, 1H), 7.48 (ddd, 1H), 7.67-7.71 (m, 1H), 7.73-7.82 (m, 2H), 8.13-8.18 (m, 2H), 8.23 (dd, 1H), 8.33 (dd, 1H), 8.47-8.51 (m, 2H).

### Example 307

### (3R)-3-({2-[4-(S-methylsulfonimidoyl)phenyl][1,2,4]triazolo[1,5-c]quinazolin-5-yl}amino)azepan-2-one, Diastereomer 1

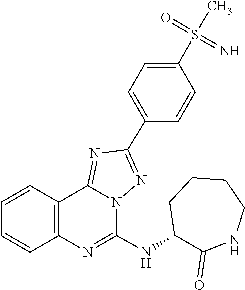 [SMILES: CS(=N)(=O)c1ccc(-c2nc3c4ccccc4nc(N[C@@H]4CCCCNC4=O)n3n2)cc1]

Chiral HPLC separation of (3R)-3-({2-[4-(methanesulfonimidoyl)phenyl][1,2,4]triazolo[1,5-c]quinazolin-5-yl}amino)azepan-2-one (125 mg, 277 μmol) (example 306) was performed (Instrument: Labomatic HD5000, Labocord-5000; Gilson GX-241, Labcol Vario 4000, Column: Cellulose SC 10μ 250×50 mm; eluent A: dichloromethane; eluent B: ethanol; isocratic: 10% B in 15 min; flow 100.0 mL/min; UV 254 nm).

Retention time of stereoisomer 1: 1.50 min; [α]²⁰_{D}: −95° (c=1) in DMSO.

Instrument: Agilent HPLC 1260; Column: Cellulose SC 3μ 100×4.6 mm; Eluent A: dichlormethane+0.1 vol-% diethylamine (99%); eluent B: ethanol; Isocratic: 10% B; flow 1.4 mL/min; temperature: 25° C.; DAD 254 nm.

### Example 308

### (3R)-3-({2-[4-(S-methylsulfonimidoyl)phenyl][1,2,4]triazolo[1,5-c]quinazolin-5-yl}amino)azepan-2-one, Diastereomer 2

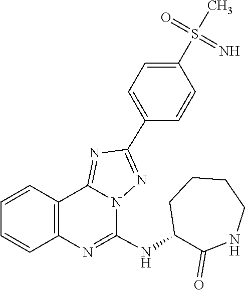 [SMILES: CS(=N)(=O)c1ccc(-c2nc3c4ccccc4nc(N[C@@H]4CCCCNC4=O)n3n2)cc1]

The title compound was prepared as described for example 307 Retention time of stereoisomer 2: 1.89 min; [α]²⁰_{D}: −52° (c=1) in DMSO.

### Example 309

### (3R)-3-({2-[3-(Trifluoromethoxy)phenyl][1,2,4]triazolo[1,5-c]quinazolin-5-yl}amino)azepan-2-one

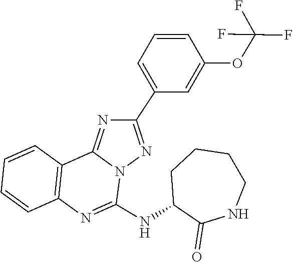 [SMILES: O=C1NCCCC[C@H]1Nc1nc2ccccc2c2nc(-c3cccc(OC(F)(F)F)c3)nn12]

5-Chloro-2-[3-(trifluoromethoxy)phenyl][1,2,4]triazolo[1,5-c]quinazoline (75.0 mg, 206 μmol) was suspended in DMSO (0.82 mL). (3R)-3-Aminoazepan-2-one (39.5 mg, 308 μmol) and N,N-diisopropylethylamine (72 μL, 410 μmol) were added. It was stirred at 60° C. After some minutes additional DMSO (0.5 mL) was added and it was stirred for 2 h at 60° C. The reaction mixture was allowed to cool down and water (4 mL) was added. The solid was filtered off and washed twice with water. It was dried under vacuum at 50° C. giving 75 mg of crude material which was purified by basic HPLC affording 44 mg (47%) of the title product.

LC-MS (Method 2): Rₜ=1.51 min; MS (ESIpos): m/z=457 [m+H]⁺

[α]²⁰_{D}: −73.7° (c=1.00, DMSO)

¹H-NMR (400 MHz, DMSO-d₆): δ [ppm]=1.27-1.39 (m, 1H), 1.53-1.65 (m, 1H), 1.81-1.95 (m, 2H), 1.98-2.08 (m, 1H), 2.28-2.36 (m, 1H), 3.12-3.22 (m, 1H), 3.32-3.41 (m, 1H and water signal), 4.85 (br dd, 1H), 7.47 (ddd, 1H), 7.60 (dt, 1H), 7.66-7.69 (m, 1H), 7.73-7.80 (m, 3H), 8.16 (s, 1H), 8.21 (dd, 1H), 8.30-8.34 (m, 2H).

The following examples were prepared analogously to example 309 and the racemic mixtures were separated by chiral HPLC:

| | Structure |
|---|---|
| | IUPAC-Name |
| | LC-MS (method): Retention time; Mass found |
| Example | ¹H-NMR |
| Example 310 | 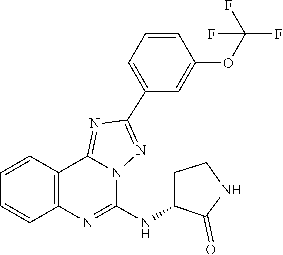 [SMILES: O=C1NCC[C@H]1Nc1nc2ccccc2c2nc(-c3cccc(OC(F)(F)F)c3)nn12] |
| | (3R)-3-({2-[3-(trifluoromethoxy)phenyl][1,2,4]triazolo[1,5-c]quinazolin-5- |
| | yl}amino)pyrrolidin-2-one |
| | LC-MS (Method 2): Rₜ = 1.31 min; MS (ESIpos): m/z = 429 [m + H]⁺ |
| | [α]²⁰ _{D}: +6.5° (c = 1.00, DMSO) |
| | ¹H-NMR (400 MHz, DMSO-d₆): δ [ppm] = 2.26-2.38 (m, 1H), 3.33-3.37 (m |
| | and water signal, 1H), 4.93-5.01 (m, 1H), 7.43-7.48 (m, 1H), 7.60 (dt, 1H), |
| | 7.63 (d, 1H), 7.72-7.80 (m, 2H), 8.02 (s, 1H), 8.19 (s, 1H), 8.30-8.35 (m, |
| | 2H), 8.38 (d, 1H). |
| Example 311 | 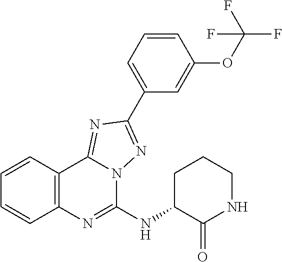 [SMILES: O=C1NCCC[C@H]1Nc1nc2ccccc2c2nc(-c3cccc(OC(F)(F)F)c3)nn12] |
| | (3R)-3-({2-[3-(trifluoromethoxy)phenyl][1,2,4]triazolo[1,5-c]quinazolin-5- |
| | yl}amino)piperidin-2-one |
| | LC-MS (Method 2): Rₜ = 1.38 min; MS (ESIpos): m/z = 443 [m + H]⁺ |
| | [α]²⁰ _{D}: −20.5° (c = 1.00, DMSO) |
| | ¹H-NMR (400 MHz, DMSO-d₆): δ [ppm] = 1.86-1.98 (m, 2H), 2.11 (qd, 1H), |
| | 2.24-2.31 (m, 1H), 3.20-3.29 (m, 2H), 4.68-4.76 (m, 1H), 7.45 (ddd, 1H), |
| | 7.60 (dt, 1H), 7.64 (d, 1H), 7.71-7.80 (m, 2H), 7.81 (br s, 1H), 8.19 (t, 1H), |
| | 8.26 (d, 1H), 8.29-8.35 (m, 2H). |
| Example 312 | 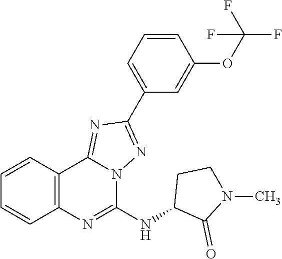 [SMILES: CN1CC[C@@H](Nc2nc3ccccc3c3nc(-c4cccc(OC(F)(F)F)c4)nn23)C1=O] |
| | (3R)-1-methyl-3-({2-[3-(trifluoromethoxy)phenyl][1,2,4]triazolo[1,5- |
| | c]quinazolin-5-yl}amino)pyrrolidin-2-one |
| | LC-MS (Method 2): Rt = 1.37 min; MS (ESIpos): m/z = 443 [m + H]⁺ |
| | [α]²⁰ _{D} : +7.1° (c = 1.00, DMSO) |
| | ¹H-NMR (400 MHz, DMSO-d₆): δ [ppm] = 2.17-2.28 (m, 1H), 2.84 (s, 3H), |
| | 3.42-3.48 (m, 2H), 4.99 (q, 1H), 7.45 (ddd, 1H), 7.58-7.64 (m, 2H), 7.72- |
| | 7.79 (m, 2H), 8.18-8.20 (m, 1H), 8.29-8.35 (m, 2H), 8.51 (d, 1H). |
| Example 313 | 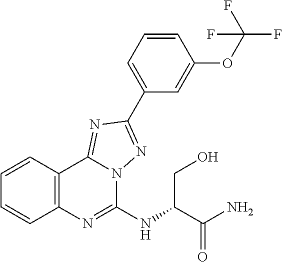 [SMILES: NC(=O)[C@@H](CO)Nc1nc2ccccc2c2nc(-c3cccc(OC(F)(F)F)c3)nn12] |
| | N²-{2-[3-(trifluoromethoxy)phenyl][1,2,4]triazolo[1,5-c]quinazolin-5-yl}- |
| | D-serinamide |
| | LC-MS (Method 2): Rₜ = 1.18 min; MS (ESIpos): m/z = 433 [m + H]⁺ |
| | [α]²⁰ _{D}: −81.1° (c = 1.00, DMSO) |
| | ¹H-NMR (400 MHz, DMSO-d₆): δ [ppm] = 3.96 (t, 2H), 4.71-4.76 (m, 1H), |
| | 5.18 (t, 1H), 7.32 (s, 1H), 7.47 (ddd, 1H), 7.58-7.69 (m, 4H), 7.73-7.80 (m, |
| | 2H), 8.20 (t, 1H), 8.31-8.38 (m, 2H). |
| Example 314 | 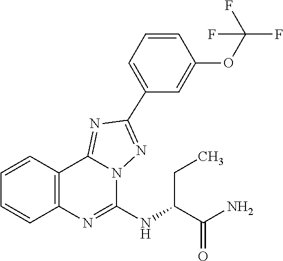 [SMILES: CC[C@@H](Nc1nc2ccccc2c2nc(-c3cccc(OC(F)(F)F)c3)nn12)C(N)=O] |
| | (2R)-2-({2-[3-(trifluoromethoxy)phenyl][1,2,4]triazolo[1,5-c]quinazolin-5- |
| | yl}amino)butanamide |
| | LC-MS (Method 2): Rₜ = 1.36 min; MS (ESIpos): m/z = 431 [m + H]⁺ |
| | [α]²⁰ _{D}: −58.6° (c = 1.00, DMSO) |
| | ¹H-NMR (400 MHz, DMSO-d₆): δ [ppm] = 0.97 (t, 3H), 1.88-2.00 (m, 1H), |
| | 2.02-2.13 (m, 1H), 4.69 (td, 1H), 7.31 (s, 1H), 7.46 (ddd, 1H), 7.58-7.62 |
| | (m, 1H), 7.64 (d, 1H), 7.68-7.79 (m, 4H), 8.20 (t, 1H), 8.32 (dd, 1H), 8.36 |
| | (dt, 1H). |
| Example 315 | 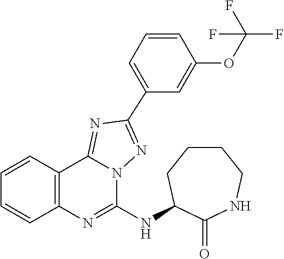 [SMILES: O=C1NCCCC[C@@H]1Nc1nc2ccccc2c2nc(-c3cccc(OC(F)(F)F)c3)nn12] |
| | (3S)-3-({2-[3-(trifluoromethoxy)phenyl][1,2,4]triazolo[1,5-c]quinazolin-5- |
| | yl}amino)azepan-2-one |
| | LC-MS (Method 2): Rₜ = 1.50 min; MS (ESIpos): m/z = 457 [m + H]⁺ |
| | [α]²⁰ _{D}: +77.6° (c = 1.00, DMSO) |
| | ¹H-NMR (400 MHz, DMSO-d₆): δ [ppm] = 1.26-1.39 (m, 1H), 1.53-1.64 (m, |
| | 1H), 1.81-1.95 (m, 2H), 1.98-2.07 (m, 1H), 2.28-2.35 (m, 1H), 3.12-3.22 |
| | (m, 1H), 3.32-3.41 (m, 1H and water signal), 4.85 (br dd, 1H), 7.44-7.50 |
| | (m, 1H), 7.60 (dt, 1H), 7.66-7.70 (m, 1H), 7.73-7.81 (m, 3H), 8.16 (s, 1H), |
| | 8.21 (br dd, 1H), 8.32 (d, 2H). |
| Example 316 | 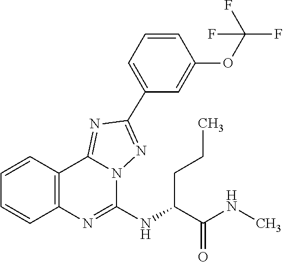 [SMILES: CCC[C@@H](Nc1nc2ccccc2c2nc(-c3cccc(OC(F)(F)F)c3)nn12)C(=O)NC] |
| | N-methyl-N²-{2-[3-(trifluoromethoxy)phenyl][1,2,4]triazolo[1,5- |
| | c]quinazolin-5-yl}-D-norvalinamide |
| | LC-MS (Method 2): Rₜ = 1.47 min; MS (ESIpos): m/z = 459 [m + H]⁺ |
| | ¹H-NMR (400 MHz, DMSO-d₆): δ [ppm] = 0.92 (t, 3H), 1.31-1.51 (m, 2H), |
| | 1.86-2.01 (m, 2H), 2.62 (d, 3H), 4.77 (td, 1H), 7.43-7.48 (m, 1H), 7.58- |
| | 7.65 (m, 2H), 7.71-7.79 (m, 2H), 7.86 (d, 1H), 8.12 (q, 1H), 8.21 (s, 1H), |
| | 8.31 (dd, 1H), 8.36 (dt, 1H). |
| Example 317 | 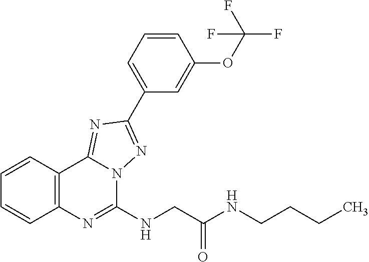 [SMILES: CCCCNC(=O)CNc1nc2ccccc2c2nc(-c3cccc(OC(F)(F)F)c3)nn12] |
| | N-butyl-N²-{2-[3-(trifluoromethoxy)phenyl][1,2,4]triazolo[1,5- |
| | c]quinazolin-5-yl}glycinamide |
| | LC-MS (Method 2): Rₜ = 1.43 min; MS (ESIpos): m/z = 459 [m + H]⁺ |
| | ¹H-NMR (400 MHz, DMSO-d₆): δ [ppm] = 0.84 (t, 3H), 1.21-1.32 (m, 2H), |
| | 1.34-1.43 (m, 2H), 3.09 (q, 2H), 4.15 (d, 2H), 7.45 (ddd, 1H), 7.58-7.63 (m, |
| | 2H), 7.71-7.80 (m, 2H), 8.04 (t, 1H), 8.18-8.20 (m, 1H), 8.30-8.36 (m, |
| | 3H). |
| Example 318 | 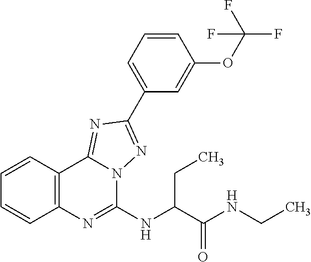 [SMILES: CCNC(=O)C(CC)Nc1nc2ccccc2c2nc(-c3cccc(OC(F)(F)F)c3)nn12] |
| | N-ethyl-2-({2-[3-(trifluoromethoxy)phenyl][1,2J4]triazolo[1,5- |
| | c]quinazolin-5-yl}amino)butanamide |
| | LC-MS (Method 2): Rₜ = 1.33 min; MS (ESIpos): m/z = 459 [m + H]⁺ |
| | ¹H-NMR (400 MHz, DMSO-d₆): δ [ppm] = 0.96 (t, 3H), 1.04 (t, 3H), 1.87-2.09 |
| | (m, 2H), 3.10-3.18 (m, 2H), 4.64-4.70 (m, 1H), 7.46 (t, 1H), 7.58-7.65 (m, |
| | 2H), 7.70-7.80 (m, 3H), 8.21 (s, 1H), 8.24 (br t, 1H), 8.32 (d, 1H), 8.36 (d, |
| | 1H). |
| Example 319 | 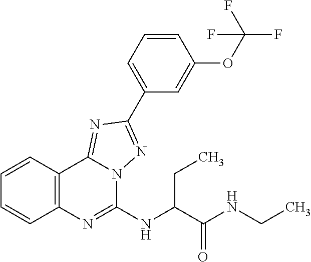 [SMILES: CCNC(=O)C(CC)Nc1nc2ccccc2c2nc(-c3cccc(OC(F)(F)F)c3)nn12] |
| | N-ethyl-2-({2-[3-(trifluoromethoxy)phenyl][1,2,4]triazolo[1,5- |
| | c]quinazolin-5-yl}amino)butanamide, enantiomer 1 |
| | LC-MS (Method 2): Rₜ = 1.33 min; MS (ESIpos): m/z = 459 [m + H]⁺ |
| | [α]²⁰ _{D}: −5.9° (c = 1.00, DMSO) |
| | ¹H-NMR (400 MHz, DMSO-d₆): δ [ppm] = 0.96 (t, 3H), 1.04 (t, 3H), 1.87-2.09 |
| | (m, 2H), 3.10-3.18 (m, 2H), 4.67 (td, 1H), 7.44-7.48 (m, 1H), 7.58-7.65 |
| | (m, 2H), 7.71-7.79 (m, 3H), 8.21 (s, 1H), 8.24 (t, 1H), 8.32 (dd, 1H), 8.36 |
| | (dt, 1H). |
| | Instrument: Labomatic HD5000, Cellulose SC 5μ 250 × 30 mm, eluent A: n- |
| | hexane + 0.1 vol % of diethylamine (99%), eluent B: ethanol, gradient: 10- |
| | 30% of eluent B in 20 min, flow 40 mL/min, UV 254 nm. |
| | Chiral HPLC: Rt = 1.44 min |
| | Instrument: Agilent HPLC 1260; column: Cellulose SC 3μ 100 × 4, 6 mm, eluent |
| | A: n-hexane + 0.1 vol % of diethylamine (99%), eluent B: ethanol, gradient: |
| | 10-30% of eluent B in 7 min, flow 1.4 mL/min; temperature: 25° C.; DAD |
| | 254 nm. |
| Example 320 | 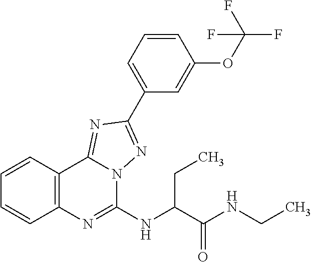 [SMILES: CCNC(=O)C(CC)Nc1nc2ccccc2c2nc(-c3cccc(OC(F)(F)F)c3)nn12] |
| | N-ethyl-2-({2-[3-(trifluoromethoxy)phenyl][1,2,4]triazolo[1,5- |
| | c]quinazolin-5-yl}amino)butanamide, enantiomer 2 |
| | LC-MS (Method 2): Rₜ = 1.33 min; MS (ESIpos): m/z = 459 [m + H]⁺ |
| | [α]²⁰ _{D}: +49.2° (c = 1.00, DMSO) |
| | ¹H-NMR (400 MHz, DMSO-d₆): δ [ppm] = 0.96 (t, 3H), 1.03 (t, 3H), 1.87-2.09 |
| | (m, 2H), 3.10-3.18 (m,2H), 4.67 (td, 1H), 7.44-7.48 (m, 1H), 7.58-7.65 |
| | (m, 2H), 7.71-7.79 (m, 3H), 8.21 (s, 1H), 8.24 (t, 1H), 8.32 (dd, 1H), 8.36 (d, |
| | 1H). |
| | Instrument: Labomatic HD5000, Cellulose SC 5μ 250 × 30 mm, eluent A: n- |
| | hexane + 0.1 vol % of diethylamine (99%), eluent B: ethanol, gradient: 10- |
| | 30% of eluent B in 20 min, flow 40 mL/min, UV 254 nm. |
| | Chiral HPLC: Rt = 2.30 min |
| | Instrument: Agilent HPLC 1260; column: Cellulose SC 3μ 100 × 4, 6 mm, eluent |
| | A: n-hexane + 0.1 vol % of diethylamine (99%), eluent B: ethanol, gradient: |
| | 10-30% of eluent B in 7 min, flow 1.4 mL/min; temperature: 25° C.; DAD |
| | 254 nm. |
| Example 321 | 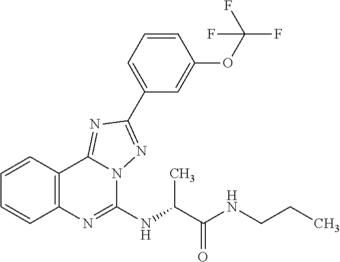 [SMILES: CCCNC(=O)[C@@H](C)Nc1nc2ccccc2c2nc(-c3cccc(OC(F)(F)F)c3)nn12] |
| | N-propyl-N2-{2-[3-(trifluoromethoxy)phenyl][1,2,4]triazolo[1,5- |
| | c]quinazolin-5-yl}-D-alaninamide |
| | LC-MS (Method 2): Rₜ = 1.47 min; MS (ESIpos): m/z = 459 [m + H]⁺ |
| | [α]²⁰ _{D}: −63.6° (c = 1.00, DMSO) |
| | ¹H-NMR (500 MHz, DMSO-d₆): δ [ppm] = 0.84 (t, 3H), 1.43 (sxt, 2H), 1.54 (d, |
| | 3H), 3.02-3.13 (m, 2H), 4.72-4.79 (m, 1H), 7.46 (t, 1H), 7.58-7.64 (m, |
| | 2H), 7.72-7.79 (m, 2H), 7.90 (d, 1H), 8.16 (t, 1H), 8.21 (s, 1H), 8.31 (dd, |
| | 1H), 8.35 (d, 1H). |
| Example 322 | 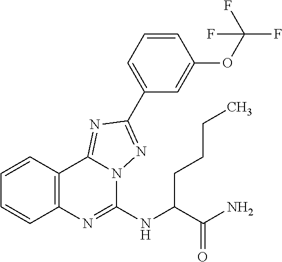 [SMILES: CCCCC(Nc1nc2ccccc2c2nc(-c3cccc(OC(F)(F)F)c3)nn12)C(N)=O] |
| | N²-{2-[3-(trifluoromethoxy)phenyl][1,2,4]triazolo[1,5-c]quinazolin-5-yl}- |
| | norleucinamide |
| | LC-MS (Method 2): Rₜ = 1.49 min; MS (ESIpos): m/z = 459 [m + H]⁺ |
| | ¹H-NMR (400 MHz, DMSO-d₆): δ [ppm] = 0.87 (t, 3H), 1.27-1.46 (m, 4H), |
| | 1.87-2.06 (m, 2H), 4.74 (td, 1H), 7.27 (s, 1H), 7.46 (ddd, 1H), 7.58-7.62 |
| | (m, 1H), 7.63 (d, 1H), 7.69 (s, 1H), 7.71-7.79 (m, 3H), 8.21 (s, 1H), 8.31 |
| | (dd, 1H), 8.36 (dt, 1H). |
| Example 323 | 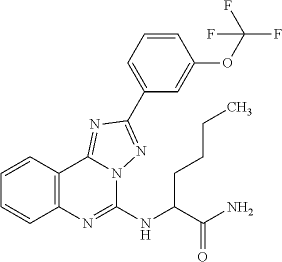 [SMILES: CCCCC(Nc1nc2ccccc2c2nc(-c3cccc(OC(F)(F)F)c3)nn12)C(N)=O] |
| | N²-{2-[3-(trifluoromethoxy)phenyl][1,2,4]triazolo[1,5-c]quinazolin-5-yl>- |
| | norleucinamide, enatiomer 1 |
| | LC-MS (Method 2): Rₜ = 1.49 min; MS (ESIpos): m/z = 459 [m + H]⁺ |
| | [α]²⁰ _{D}: +49.4° (c = 1.00, DMSO) |
| | ¹H-NMR (400 MHz, DMSO-d₆): δ [ppm] = 0.87 (t, 3H), 1.27-1.46 (m, 4H), |
| | 1.87-2.06 (m, 2H), 4.73 (td, 1H), 7.27 (s, 1H), 7.43-7.48 (m, 1H), 7.58- |
| | 7.62 (m, 1H), 7.64 (d, 1H), 7.69 (s, 1H), 7.72-7.79 (m, 3H), 8.21 (s, 1H), |
| | 8.32 (dd, 1H), 8.36 (d, 1H). |
| | Instrument: Labomatic HD5000, Amylose SA 5μ 250 × 30 mm, eluent A: 2- |
| | methoxy-2-methylpropane, eluent B: acetonitrile, isocratic 50% eluent B, flow |
| | 60 mL/min, UV 254 nm. |
| | Chiral HPLC: Rt = 1.44 min |
| | Instrument: Agilent HPLC 1260; column: Amylose SA 3μ 100 × 4, 6 mm, eluent |
| | A: 2-methoxy-2-methylpropane + 0.1 vol % of diethylamine (99%), eluent B: |
| | acetonitrile, isocratic: 50% B, flow 1.4 mL/min; temperature: 25° C.; DAD 254 |
| | nm. |
| Example 324 | 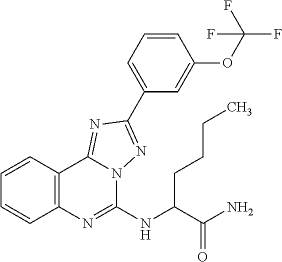 [SMILES: CCCCC(Nc1nc2ccccc2c2nc(-c3cccc(OC(F)(F)F)c3)nn12)C(N)=O] |
| | N²-{2-[3-(trifluoromethoxy)phenyl][1,2,4]triazolo[1,5-c]quinazolin-5-yl}- |
| | norleucinamide, enantiomer 2 |
| | LC-MS (Method 2): Rₜ = 1.49 min; MS (ESIpos): m/z = 459 [m + H]⁺ |
| | [α]²⁰ _{D}: −52.6° (c = 1.00, DMSO) |
| | ¹H-NMR (400 MHz, DMSO-d₆): δ [ppm] = 0.87 (t, 3H), 1.27-1.46 (m, 4H), |
| | 1.87-2.07 (m, 2H), 4.73 (td, 1H), 7.27 (s, 1H), 7.43-7.48 (m, 1H), 7.60 (dt, |
| | 1H), 7.64 (d, 1H), 7.69 (s, 1H), 7.72-7.79 (m, 3H), 8.21 (s, 1H), 8.32 (dd, |
| | 1H), 8.34-8.38 (m, 1H). |
| | Instrument: Labomatic HD5000, Amylose SA 5μ 250 x 30 mm, eluent A: 2- |
| | methoxy-2-methylpropane, eluent B: acetonitrile, isocratic 50% eluent B, flow |
| | 60 mL/min, UV 254 nm. |
| | Chiral HPLC: Rt = 2.01 min |
| | Instrument: Agilent HPLC 1260; column: Amylose SA 3μ 100 x 4, 6 mm, eluent |
| | A: 2-methoxy-2-methylpropane + 0.1 vol % of diethylamine (99%), eluent B: |
| | acetonitrile, isocratic: 50% B, flow 1.4 mL/min; temperature: 25° C.; DAD 254 |
| | nm. |
| Example 325 | 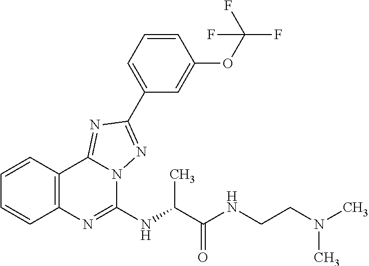 [SMILES: C[C@@H](Nc1nc2ccccc2c2nc(-c3cccc(OC(F)(F)F)c3)nn12)C(=O)NCCN(C)C] |
| | N-[2-(dimethylamino)ethyl]-N²-{2-[3- |
| | (trifluoromethoxy)phenyl][1,2,4]triazolo[1,5-c]quinazolin-5-yl}-D- |
| | alaninamide |
| | LC-MS (Method 2): Rₜ = 1.40 min; MS (ESIpos): m/z = 488 [m + H]⁺ |
| | [α]²⁰ _{D}: −60.4° (c = 1.00, DMSO) |
| | ¹H-NMR (400 MHz, DMSO-d₆): δ [ppm] = 1.53 (d, 3H), 2.10 (s, 6H), 2.24- |
| | 2.31 (m, 2H), 3.11-3.27 (m, 2H), 4.77 (quin, 1H), 7.46 (ddd, 1H), 7.58-7.65 |
| | (m, 2H), 7.72-7.79 (m, 2H), 7.94 (d, 1H), 8.13 (t, 1H), 8.21 (t, 1H), 8.32 (dd, |
| | 1H), 8.35 (dt, 1H). |

### Example 326

### (3R)-3-({2-[4-(Trifluoromethoxy)phenyl][1,2,4]triazolo[1,5-c]quinazolin-5-yl}amino)azepan-2-one

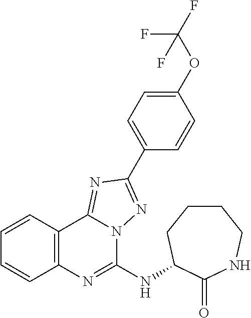 [SMILES: O=C1NCCCC[C@H]1Nc1nc2ccccc2c2nc(-c3ccc(OC(F)(F)F)cc3)nn12]

5-Chloro-2-[4-(trifluoromethoxy)phenyl][1,2,4]triazolo[1,5-c]quinazoline (60.0 mg, 165 μmol) and was suspended in DMSO (0.655 mL). (3R)-3-Aminoazepan-2-one (31.6 mg, 308 μmol) and N,N-diisopropylethylamine (57 μL, 330 μmol) were added. It was stirred at 60° C. After 2 h additional DMSO (0.6 mL) was added and the reaction mixture was allowed to cool down and was stirred over the weekend at rt. Water (15 mL) was added to the reaction mixture and was stirred for 15 min. The solid was filtered off and washed twice with water. It was dried under vacuum at 50° C. affording 65 mg of crude material which was crystallized from DMSO. The solid material was filtered off, washed with DMSO (2×0.5 mL) and twice with water, dried under vacuum at 50° C. to obtain 37 mg (49%) of the title compound.

LC-MS (Method 2): Rₜ=1.50 min; MS (ESIpos): m/z=457 [m+H]⁺

[α]²⁰_{D}: −70.3° (c=1.00, DMSO)

¹H-NMR (400 MHz, DMSO-d₆): δ [ppm]=1.26-1.39 (m, 1H), 1.51-1.63 (m, 1H), 1.81-1.95 (m, 2H), 1.98-2.08 (m, 1H), 2.29-2.37 (m, 1H), 3.12-3.22 (m, 1H), 3.32-3.41 (m, 1H and water signal), 4.84 (br dd, 1H), 7.44-7.49 (m, 1H), 7.60 (d, 2H), 7.66-7.69 (m, 1H), 7.73-7.78 (m, 2H), 8.23 (dd, 1H), 8.31 (dd, 1H), 8.38-8.43 (m, 2H).

The following examples were prepared analogously to example 326 and the racemic mixtures were separated by chiral HPLC:

| | Structure |
|---|---|
| | IUPAC-Name |
| | LC-MS (method): Retention time; Mass found |
| Example | ¹H-NMR |
| Example 327 | 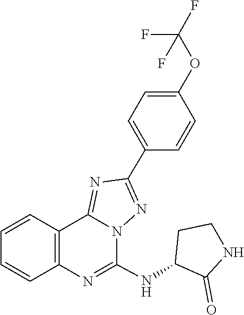 [SMILES: O=C1NCC[C@H]1Nc1nc2ccccc2c2nc(-c3ccc(OC(F)(F)F)cc3)nn12] |
| | (3R)-3-({2-[4-(trifluoromethoxy)phenyl][1,2,4]triazolo[1,5-c]quinazolin-5- |
| | yl}amino)pyrrolidin-2-one |
| | LC-MS (Method 2): Rₜ = 1.30 min; MS (ESIpos): m/z = 429 [m + H] |
| | [α]²⁰_{D}: +8.9° (c = 1.00, DMSO) |
| | ¹H-NMR (400 MHz, DMSO-d₆): δ [ppm] = 2.26-2.38 (m, 1H), 3.34-3.37 (m, |
| | 1H), 4.90-4.99 (m, 1H), 7.45 (td, 1H), 7.60-7.65 (m, 3H), 7.72-7.77 (m, |
| | 1H), 8.01 (s, 1H), 8.30 (dd, 1H), 8.34 (d, 1H), 8.39-8.44 (m, 2H). |
| Example 328 | 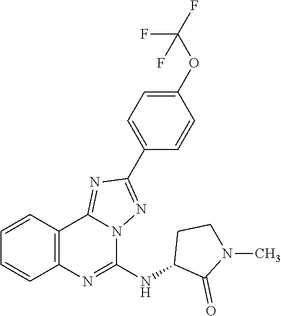 [SMILES: CN1CC[C@@H](Nc2nc3ccccc3c3nc(-c4ccc(OC(F)(F)F)cc4)nn23)C1=O] |
| | (3R)-1-methyl-3-({2-[4-(trifluoromethoxy)phenyl][1,2,4]triazolo[1,5- |
| | c]quinazolin-5-yl}amino)pyrrolidin-2-one |
| | LC-MS (Method 2): Rₜ = 1.37 min; MS (ESIpos): m/z = 443 [m + H]⁺ |
| | [α]²⁰_{D}: +6.9° (c = 1.00, DMSO) |
| | ¹H-NMR (400 MHz, DMSO-d₆): δ [ppm] = 2.17-2.29 (m, 1H), 2.83 (s, 3H), |
| | 3.41-3.48 (m, 2H), 4.98 (q, 1H), 7.45 (t, 1H), 7.61 (br d, 3H), 7.71-7.76 (m, |
| | 1H), 8.30 (d, 1H), 8.40 (d, 2H), 8.48 (d, 1H). |
| Example 329 | 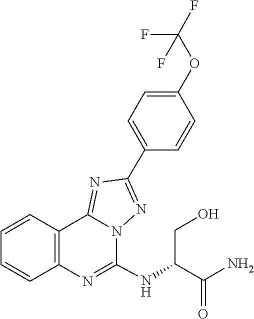 [SMILES: NC(=O)[C@@H](CO)Nc1nc2ccccc2c2nc(-c3ccc(OC(F)(F)F)cc3)nn12] |
| | N²-{2-[4-(trifluoromethoxy)phenyl][1,2,4]triazolo[1,5-c]quinazolin-5-yl}- |
| | D-serinamide |
| | LC-MS (Method 2): Rₜ = 1.19 min; MS (ESIpos): m/z = 433 [m + H]⁺ |
| | [α]²⁰_{D}: −71.4° (c = 1.00, DMSO) |
| | ¹H-NMR (400 MHz, DMSO-d₆): δ [ppm] = 3.96 (t, 2H), 4.70-4.75 (m, 1H), |
| | 5.18 (t, 1H), 7.33 (s, 1H), 7.47 (t, 1H), 7.55 (d, 1H), 7.61 (br d, 2H), 7.65 (d, |
| | 1H), 7.68 (s, 1H), 7.72-7.78 (m, 1H), 8.32 (d, 1H), 8.43 (d, 2H). |
| Example 330 | 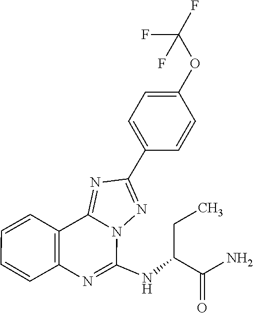 [SMILES: CC[C@@H](Nc1nc2ccccc2c2nc(-c3ccc(OC(F)(F)F)cc3)nn12)C(N)=O] |
| | (2R)-2-({2-[4-(trifluoromethoxy)phenyl][1,2,4]triazolo[1,5-c]quinazolin-5- |
| | yl}amino)butanamide |
| | LC-MS (Method 2): Rₜ = 1.37 min; MS (ESIpos): m/z = 431 [m + H]⁺ |
| | [α]²⁰_{D}: −64.9° (c = 1.00, DMSO) |
| | ¹H-NMR (400 MHz, DMSO-d₆): δ [ppm] = 0.96 (t, 3H), 1.88-2.00 (m, 1H), |
| | 2.02-2.14 (m, 1H), 4.70 (td, 1H), 7.33 (s, 1H), 7.43-7.48 (m, 1H), 7.57- |
| | 7.66 (m, 4H), 7.70-7.77 (m, 2H), 8.30 (dd, 1H), 8.40-8.46 (m, 2H). |
| Example 331 | 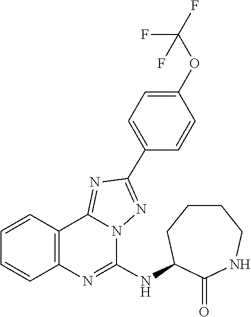 [SMILES: O=C1NCCCC[C@@H]1Nc1nc2ccccc2c2nc(-c3ccc(OC(F)(F)F)cc3)nn12] |
| | (3S)-3-({2-[4-(trifluoromethoxy)phenyl][1,2,4]triazolo[1,5-c]quinazolin-5- |
| | yl}amino)azepan-2-one |
| | LC-MS (Method 2): Rₜ = 1.50 min; MS (ESIpos): m/z = 457 [m + H]⁺ |
| | [α]²⁰_{D}: +74.4° (c = 1.00, DMSO) |
| | ¹H-NMR (400 MHz, DMSO-d₆): δ [ppm] = 1.26-1.39 (m, 1H), 1.51-1.63 (m, |
| | 1H), 1.81-1.95 (m, 2H), 1.98-2.08 (m, 1H), 2.28-2.37 (m, 1H), 3.12-3.22 |
| | (m, 1H), 3.32-3.42 (m, 1H and water signal), 4.84 (br dd, 1H), 7.47 (ddd, |
| | 1H), 7.58-7.62 (m, 2H), 7.66-7.69 (m, 1H), 7.73-7.78 (m, 2H), 8.22 (dd, |
| | 1H), 8.31 (dd, 1H), 8.38-8.43 (m, 2H). |
| Example 332 | 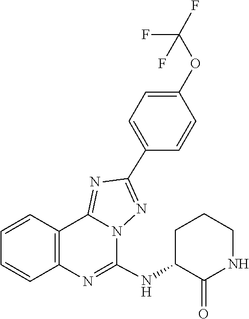 [SMILES: O=C1NCCC[C@H]1Nc1nc2ccccc2c2nc(-c3ccc(OC(F)(F)F)cc3)nn12] |
| | (3R)-3-({2-[4-(trifluoromethoxy)phenyl][1,2,4]triazolo[1,5-c]quinazolin-5- |
| | yl}amino)piperidin-2-one |
| | LC-MS (Method 2): Rₜ = 1.38 min; MS (ESIpos): m/z = 443 [m + H]⁺ |
| | [α]²⁰_{D}: −29.3° (c = 1.00, DMSO) |
| | ¹H-NMR (400 MHz, DMSO-d₆): δ [ppm] = 1.84-1.98 (m, 2H), 2.10 (qd, 1H), |
| | 2.27-2.35 (m, 1H), 3.21-3.29 (m, 2H), 4.70 (dt, 1H), 7.42-7.47 (m, 1H), |
| | 7.58-7.66 (m, 3H), 7.71-7.76 (m, 1H), 7.82 (br s, 1H), 8.22 (d, 1H), 8.30 |
| | (dd, 1H), 8.38-8.43 (m, 2H). |

### Example 333

### (3R)-3-{[2-(4-Methoxythiophen-3-yl)[1,2,4]triazolo[1,5-c]quinazolin-5-yl]amino}azepan-2-one

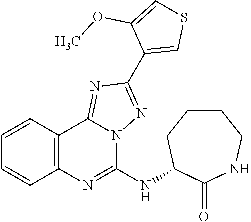 [SMILES: COc1cscc1-c1nc2c3ccccc3nc(N[C@@H]3CCCCNC3=O)n2n1]

5-Chloro-2-(4-methoxy-3-thienyl)[1,2,4]triazolo[1,5-c]quinazoline (74.0 mg, 234 μmol) was suspended in DMSO (0.93 mL). (3R)-3-Aminoazepan-2-one (44.9 mg, 350 μmol) and N,N-diisopropylethylamine (81 μL, 470 μmol) were added. It was stirred at 60° C. for 2 h. The reaction mixture was allowed to cool down and DMSO (1 mL) was added. The crude product was purified by HPLC to obtain 9 mg (9%) of the title compound.

LC-MS (Method 2): Rₜ=1.19 min; MS (ESIpos): m/z=409 [m+H]⁺

[α]²⁰_{D}: −74.4° (c=1.00, DMSO)

¹H-NMR (400 MHz, DMSO-d₆): δ [ppm]=1.25-1.38 (m, 1H), 1.49-1.61 (m, 1H), 1.81-1.95 (m, 2H), 1.97-2.07 (m, 1H), 2.28-2.36 (m, 1H), 3.11-3.21 (m, 1H), 3.32-3.41 (m, 1H and water signal), 3.89 (s, 3H), 4.83 (br dd, 1H), 6.84 (d, 1H), 7.42-7.47 (m, 1H), 7.63 (d, 1H), 7.65-7.68 (m, 1H), 7.71-7.76 (m, 1H), 8.17-8.24 (m, 2H), 8.26 (dd, 1H).

The following examples were prepared analogously to example 333 and the racemic mixtures were separated by chiral HPLC:

| | Structure |
|---|---|
| | IUPAC-Name |
| | LC-MS (method): Retention time; Mass found |
| Example | ¹H-NMR |
| Example 334 | 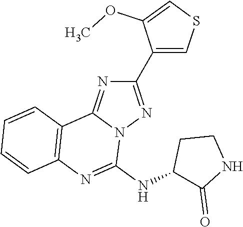 [SMILES: COc1cscc1-c1nc2c3ccccc3nc(N[C@@H]3CCNC3=O)n2n1] |
| | (3R)-3-{[2-(4-methoxythiophen-3-yl)[1,2,4]triazolo[1,5-c]quinazolin-5- |
| | yl]amino}pyrrolidin-2-one |
| | LC-MS (Method 2): Rₜ = 0.97 min; MS (ESIpos): m/z = 381 [m + H]⁺ |
| | [α]²⁰_{D}: +2.4° (c = 1.00, DMSO) |
| | ¹H-NMR (400 MHz, DMSO-d₆): δ [ppm] = 2.26-2.37 (m, 1H), 3.30-3.32 (m, |
| | 1H), 3.89 (s, 3H), 4.88-4.96 (m, 1H), 6.83 (d, 1H), 7.43 (ddd, 1H), 7.62 (d, |
| | 1H), 7.72 (ddd, 1H), 7.99 (s, 1H), 8.09 (d, 1H), 8.19 (d, 1H), 8.25 (dd, 1H). |
| Example 335 | 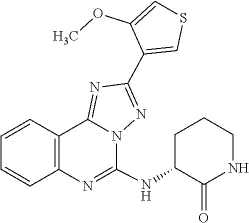 [SMILES: COc1cscc1-c1nc2c3ccccc3nc(N[C@@H]3CCCNC3=O)n2n1] |
| | (3R)-3-{[2-(4-methoxythiophen-3-yl)[1,2,4]triazolo[1,5-c]quinazolin-5- |
| | yl]amino}piperidin-2-one |
| | LC-MS (Method 2): Rₜ = 1.02 min; MS (ESIpos): m/z = 395 [m + H]⁺ |
| | [α]²⁰_{D}: −39.0° (c = 1.00, DMSO) |
| | ¹H-NMR (400 MHz, DMSO-d₆): δ [ppm] = 1.85-1.97 (m, 2H), 2.03-2.14 (m, |
| | 1H), 2.27-2.36 (m, 1H), 3.19-3.30 (m, 2H), 3.90 (s, 3H), 4.68 (dt, 1H), 6.83 |
| | (d, 1H), 7.42 (ddd, 1H), 7.61-7.64 (m, 1H), 7.72 (ddd, 1H), 7.80 (br s, 1H), |
| | 7.97 (d, 1H), 8.20 (d, 1H), 8.25 (dd, 1H). |
| Example 336 | 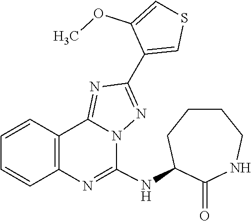 [SMILES: COc1cscc1-c1nc2c3ccccc3nc(N[C@H]3CCCCNC3=O)n2n1] |
| | (3S)-3-{[2-(4-methoxythiophen-3-yl)[1,2,4]triazolo[1,5-c]quinazolin-5- |
| | yl]amino}azepan-2-one |
| | LC-MS (Method 2): Rₜ = 1.12 min; MS (ESIpos): m/z = 409 [m + H]⁺ |
| | [α]²⁰_{D}: +79.0° (c = 1.00, DMSO) |
| | ¹H-NMR (400 MHz, DMSO-d₆): δ [ppm] = 1.25-1.39 (m, 1H), 1.48-1.61 (m, |
| | 1H), 1.80-1.95 (m, 2H), 1.97-2.07 (m, 1H), 2.28-2.37 (m, 1H), 3.11-3.21 |
| | (m, 1H), 3.32-3.41 (m, 1H and water signal), 3.89 (s, 3H), 4.83 (dd, 1H), |
| | 6.84 (d, 1H), 7.44 (ddd, 1H), 7.63 (d, 1H), 7.65-7.68 (m, 1H), 7.73 (ddd, |
| | 1H), 8.17-8.23 (m, 2H), 8.26 (dd, 1H). |
| Example 337 | 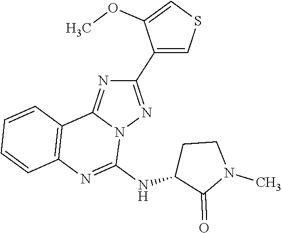 [SMILES: COc1cscc1-c1nc2c3ccccc3nc(N[C@@H]3CCN(C)C3=O)n2n1] |
| | (3R)-3-{[2-(4-methoxythiophen-3-yl)[1,2,4]triazolo[1,5-c]quinazolin-5- |
| | yl]amino}-1-methylpyrrolidin-2-one |
| | LC-MS (Method 2): Rₜ = 1.07 min; MS (ESIpos): m/z = 395 [m + H]⁺ |
| | [α]²⁰_{D}: +9.2° c = 1.00, DMSO) |
| | ¹H-NMR (400 MHz, DMSO-d₆): δ [ppm] = 2.23 (dq, 1H), 2.43-2.54 (m, 1H |
| | and DMSO signal), 2.83 (s, 3H), 3.43 (dd, 2H), 3.89 (s, 3H), 4.92-5.00 (m, |
| | 1H), 6.83 (d, 1H), 7.43 (ddd, 1H), 7.60 (d, 1H), 7.72 (ddd, 1H), 8.19 (d, 1H), |
| | 8.21-8.28 (m, 2H). |
| Example 338 | 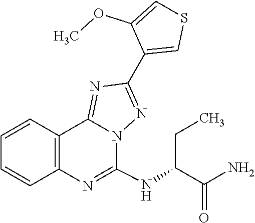 [SMILES: CC[C@@H](Nc1nc2ccccc2c2nc(-c3cscc3OC)nn12)C(N)=O] |
| | (2R)-2-{[2-(4-methoxythiophen-3-yl)[1,2,4]triazolo[1,5-c]quinazolin-5- |
| | yl]amino}butanamide |
| | LC-MS (Method 2): Rₜ = 1.06 min; MS (ESIpos): m/z = 383 [M + H]⁺ |
| | [α]²⁰_{D}: −58.4° (c = 1.00, DMSO) |
| | ¹H-NMR (400 MHz, DMSO-d₆): δ [ppm] = 0.94 (t, 3H), 1.86-1.98 (m, 1H), |
| | 2.01-2.13 (m, 1H), 3.90 (s, 3H), 4.68-4.75 (m, 1H), 6.84 (d, 1H), 7.32- |
| | 7.38 (m, 2H), 7.44 (ddd, 1H), 7.61-7.65 (m, 1H), 7.70-7.77 (m, 2H), 8.22 |
| | (d, 1H), 8.26 (dd, 1H). |
| Example 339 | 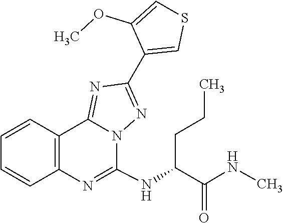 [SMILES: CCC[C@@H](Nc1nc2ccccc2c2nc(-c3cscc3OC)nn12)C(=O)NC] |
| | N²-[2-(4-methoxythiophen-3-yl)[1,2,4]triazolo[1,5-c]quinazolin-5-yl]-N- |
| | methyl-D-norvalinamide |
| | LC-MS (Method 2): Rₜ = 1.19 min; MS (ESIpos): m/z = 411 [m + H]⁺ |
| | ¹H-NMR (400 MHz, DMSO-d₆): δ [ppm] = 0.91 (t, 3H), 1.30-1.48 (m, 2H), |
| | 1.82-1.99 (m, 2H), 2.63 (d, 3H), 3.90 (s, 3H), 4.78 (td, 1H), 6.84 (d, 1H), |
| | 7.40-7.49 (m, 2H), 7.62 (d, 1H), 7.72 (ddd, 1H), 8.14-8.22 (m, 2H), 8.25 |
| | (dd, 1H). |
| Example 340 | 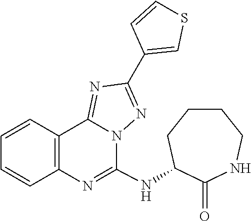 [SMILES: O=C1NCCCC[C@H]1Nc1nc2ccccc2c2nc(-c3ccsc3)nn12] |
| | (3R)-3-{[2-(thiophen-3-yl)[1,2,4]triazolo[1,5-c]quinazolin-5- |
| | yl]amino}azepan-2-one |
| | LC-MS (Method 2): Rₜ = 1.26 min; MS (ESIpos): m/z = 379 [m + H]⁺ |
| | [α]²⁰_{D}: −83.4° (c = 1.00, DMSO) |
| | ¹H-NMR (400 MHz, DMSO-d₆): δ [ppm] = 1.25-1.39 (m, 1H), 1.50-1.62 (m, |
| | 1H), 1.81-1.95 (m, 2H), 1.98-2.07 (m, 1H), 2.27-2.36 (m, 1H), 3.11-3.21 |
| | (m, 1H), 3.32-3.41 (m, 1H and water signal), 4.83 (br dd, 1H), 7.45 (ddd, |
| | 1H), 7.65-7.70 (m, 2H), 7.74 (ddd, 1H), 7.80 (dd, 1H), 7.79-7.81 (m, 1H), |
| | 8.21 (dd, 1H), 8.28 (dd, 1H), 8.38 (dd, 1H). |
| Example 341 | 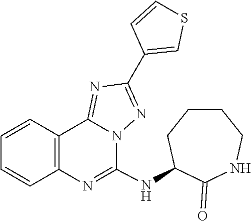 [SMILES: O=C1NCCCC[C@@H]1Nc1nc2ccccc2c2nc(-c3ccsc3)nn12] |
| | (3S)-3-{[2-(thiophen-3-yl[1,2,4]triazolo[1,5-c]quinazolin-5- |
| | yl]amino}azepan-2-one |
| | LC-MS (Method 2): Rₜ = 1.25 min; MS (ESIpos): m/z = 379 [m + H]⁺ |
| | [α]²⁰_{D}: +85.4° (c = 1.00, DMSO) |
| | ¹H-NMR (400 MHz, DMSO-d₆): δ [ppm] = 1.25-1.39 (m, 1H), 1.50-1.62 (m, |
| | 1H), 1.81-1.95 (m, 2H), 1.98-2.06 (m, 1H), 2.28-2.35 (m, 1H), 3.11-3.21 |
| | (m, 1H), 3.32-3.41 (m, 1H and water signal), 4.83 (dd, 1H), 7.45 (ddd, 1H), |
| | 7.65-7.70 (m, 2H), 7.74 (ddd, 1H), 7.77 (dd, 1H), 7.80 (dd, 1H), 8.21 (dd, |
| | 1H), 8.28 (dd, 1H), 8.38 (dd, 1H). |
| Example 342 | 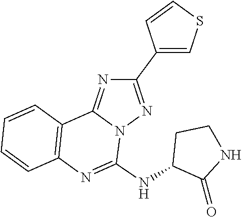 [SMILES: O=C1NCC[C@H]1Nc1nc2ccccc2c2nc(-c3ccsc3)nn12] |
| | (3R)-3-{[2-(thiophen-3-yl)[1,2,4]triazolo[1,5-c]quinazolin-5- |
| | yl]amino}pyrrolidin-2-one |
| | LC-MS (Method 2): Rₜ = 1.03 min; MS (ESIpos): m/z = 351 [m + H]⁺ |
| | [α]²⁰_{D}: +7.3° (c = 1.00, DMSO) |
| | ¹H-NMR (400 MHz, DMSO-d₆): δ [ppm] = 22.6-2.37 (m, 1H), 3.28-3.38 (m, |
| | 2H and water signal), 4.93 (dt, 1H), 7.43 (ddd, 1H), 7.60-7.64 (m, 1H), 7.70- |
| | 7.75 (m, 1H), 7.77-7.80 (m, 1H), 7.80-7.82 (m, 1H), 8.00 (s, 1H), 8.24 (d, |
| | 1H), 8.28 (dd, 1H), 8.35 (dd, 1H). |
| Example 343 | 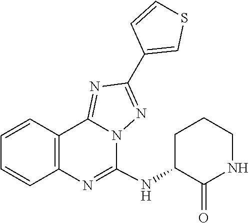 [SMILES: O=C1NCCC[C@H]1Nc1nc2ccccc2c2nc(-c3ccsc3)nn12] |
| | (3R)-3-{[2-(thiophen-3-yl)[1,2,4]triazolo[1,5-c]quinazolin-5- |
| | yl]amino}piperidin-2-one |
| | LC-MS (Method 2): Rₜ = 1.11 min; MS (ESIpos): m/z = 365 [m + H]⁺ |
| | [α]²⁰_{D}: −28.6° (c = 1.00, DMSO) |
| | ¹H-NMR (400 MHz, DMSO-d₆): δ [ppm] = 1.83-1.98 (m, 2H), 2.09 (dq, 1H), |
| | 2.26-2.35 (m, 1H), 3.19-3.29 (m, 2H), 4.69 (dt, 1H), 7.43 (ddd, 1H), 7.63 |
| | (d, 1H), 7.72 (ddd, 1H), 7.77-7.82 (m, 3H), 8.10 (d, 1H), 8.27 (dd, 1H), 8.35 |
| | (dd, 1H). |
| Example 344 | 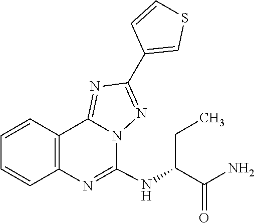 [SMILES: CC[C@@H](Nc1nc2ccccc2c2nc(-c3ccsc3)nn12)C(N)=O] |
| | (2R)-2-{[2-(thiophen-3-yl[1,2,4]triazolo[1,5-c]quinazolin-5- |
| | yl]amino}butanamide |
| | LC-MS (Method 2): Rₜ = 1.11 min; MS (ESIpos): m/z = 353 [m + H]⁺ |
| | [α]²⁰_{D}: −41.2° (c = 1.00, DMSO) |
| | ¹H-NMR (400 MHz, DMSO-d₆): δ [ppm] = 0.95 (t, 3H), 1.86-1.99 (m, 1H), |
| | 2.01-2.12 (m, 1H), 4.70 (td, 1H), 7.32 (br s, 1H), 7.44 (ddd, 1H), 7.51 (d, |
| | 1H), 7.61-7.64 (m, 1H), 7.70-7.75 (m, 2H), 7.78 (dd, 1H), 7.83 (dd, 1H), |
| | 8.28 (dd, 1H), 8.38 (dd, 1H). |
| Example 345 | 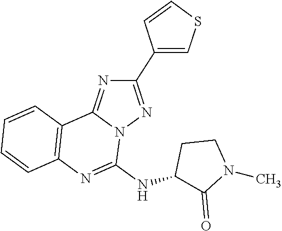 [SMILES: CN1CC[C@@H](Nc2nc3ccccc3c3nc(-c4ccsc4)nn23)C1=O] |
| | (3R)-1-methyl-3-{[2-(thiophen-3-yl)[1,2,4]triazolo[1,5-c]quinazolin-5- |
| | yl]amino}pyrrolidin-2-one |
| | LC-MS (Method 2): Rₜ = 1.11 min; MS (ESIpos): m/z = 365 [M + H]⁺ |
| | [α]²⁰_{D}: +11.6° (c = 1.00, DMSO) |
| | ¹H-NMR (400 MHz, DMSO-d₆): δ [ppm] = 2.17-2.28 (m, 1H), 2.83 (s, 3H), |
| | 3.41-3.46 (m, 2H), 4.96 (q, 1H), 7.41-7.46 (m, 1H), 7.60 (d, 1H), 7.73 (ddd, |
| | 1H), 7.77-7.83 (m, 2H), 8.27 (dd, 1H), 8.34 (dd, 1H), 8.38 (d, 1H). |
| Example 346 | 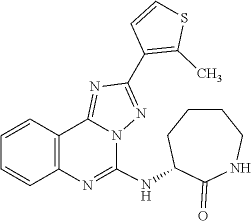 [SMILES: Cc1sccc1-c1nc2c3ccccc3nc(N[C@@H]3CCCCNC3=O)n2n1] |
| | (3R)-3-{[2-(2-methylthiophen-3-yl)[1,2,4]triazolo[1,5-c]quinazolin-5- |
| | yl]amino}azepan-2-one |
| | LC-MS (Method 2): Rₜ = 1.38 min; MS (ESIpos): m/z = 393 [m + H]⁺ |
| | [α]²⁰_{D}: −85.7° (c = 1.00, DMSO) |
| | ¹H-NMR (500 MHz, DMSO-d₆): δ [ppm] = 1.26-1.37 (m, 1H), 1.51-1.62 (m, |
| | 1H), 1.82-1.94 (m, 2H), 1.99-2.06 (m, 1H), 2.30-2.37 (m, 1H), 2.93 (s, |
| | 3H), 3.12-3.20 (m, 1H), 3.32-3.40 (m, 1H and water signal), 4.83 (br dd, |
| | 1H), 7.43-7.47 (m, 2H), 7.65-7.69 (m, 2H), 7.71-7.76 (m, 2H), 8.19 (dd, |
| | 1H), 8.28 (dd, 1H). |
| Example 347 | 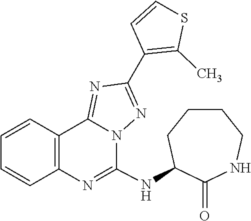 [SMILES: Cc1sccc1-c1nc2c3ccccc3nc(N[C@H]3CCCCNC3=O)n2n1] |
| | (3S)-3-{[2-(2-methylthiophen-3-yl[1,2,4]triazolo[1,5-c]quinazolin-5- |
| | yl]amino}azepan-2-one |
| | LC-MS (Method 2): Rₜ = 1.40 min; MS (ESIpos): m/z = 393 [m + H]⁺ |
| | [α]²⁰_{D}: +88.2° (c = 1.00, DMSO) |
| | ¹H-NMR (500 MHz, DMSO-d₆): δ [ppm] = 1.27-1.37 (m, 1H), 1.52-1.62 (m, |
| | 1H), 1.82-1.94 (m, 2H), 1.99-2.06 (m, 1H), 2.30-2.37 (m, 1H), 2.93 (s, |
| | 3H), 3.13-3.20 (m, 1H), 3.32-3.40 (m, 1H and water signal), 4.83 (br dd, |
| | 1H), 7.43-7.47 (m, 2H), 7.65-7.69 (m, 2H), 7.71-7.76 (m, 2H), 8.19 (dd, |
| | 1H), 8.28 (dd, 1H). |
| Example 348 | 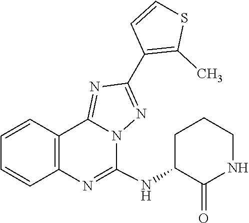 [SMILES: Cc1sccc1-c1nc2c3ccccc3nc(N[C@@H]3CCCNC3=O)n2n1] |
| | (3R)-3-{[2-(2-methylthiophen-3-yl)[1,2,4]triazolo[1,5-c]quinazolin-5- |
| | yl]amino}piperidin-2-one |
| | LC-MS (Method 2): Rₜ = 1.26 min; MS (ESIpos): m/z = 379 [m + H]⁺ |
| | [α]²⁰_{D}: −18.6° (c = 1.00, DMSO) |
| | ¹H-NMR (400 MHz, DMSO-d₆): δ [ppm] = 1.83-1.98 (m, 2H), 2.05-2.16 (m, |
| | 1H), 2.25-2.35 (m, 1H), 2.95 (s, 3H), 3.20-3.30 (m, 2H), 4.70 (dt, 1H), 7.40- |
| | 7.47 (m, 2H), 7.61-7.65 (m, 1H), 7.68-7.75 (m, 2H), 7.81 (br s, 1H), 8.10 |
| | (d, 1H), 8.27 (d, 1H). |
| Example 349 | 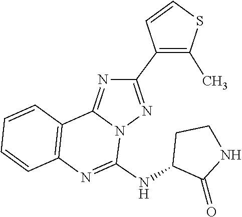 [SMILES: Cc1sccc1-c1nc2c3ccccc3nc(N[C@@H]3CCNC3=O)n2n1] |
| | (3R)-3-{[2-(2-methylthiophen-3-yl)[1,2,4]triazolo[1,5-c]quinazolin-5- |
| | yl]amino}pyrrolidin-2-one |
| | LC-MS (Method 2): Rₜ = 1.18 min; MS (ESIpos): m/z = 365 [m + H]⁺ |
| | [α]²⁰_{D}: +7.8° (c = 1.00, DMSO) |
| | ¹H-NMR (400 MHz, DMSO-d₆): δ [ppm] = 2.26-2.38 (m, 1H), 2.96 (s, 3H), |
| | 4.91-4.99 (m, 1H), 7.40-7.48 (m, 2H), 7.62 (d, 1H), 7.68-7.75 (m, 2H), |
| | 8.01 (s, 1H), 8.20 (d, 1H), 8.28 (dd, 1H). |
| Example 350 | 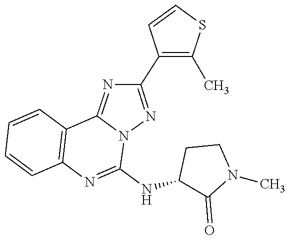 [SMILES: Cc1sccc1-c1nc2c3ccccc3nc(N[C@@H]3CCN(C)C3=O)n2n1] |
| | (3R)-1-methyl-3-{[2-(2-methylthiophen-3-yl[1,2,4]triazolo[1,5- |
| | c]quinazolin-5-yl]amino}pyrrolidin-2-one |
| | LC-MS (Method 2): Rₜ = 1.22 min; MS (ESIpos): m/z = 379 [m + H]⁺ |
| | [α]²⁰_{D}: +11.8° (c = 1.00, DMSO) |
| | ¹H-NMR (400 MHz, DMSO-d₆): δ [ppm] = 2.17-2.30 (m, 1H), 2.83 (s, 3H), |
| | 2.96 (s, 3H), 3.45 (dd, 2H), 4.98 (q, 1H), 7.40-7.47 (m, 2H), 7.60 (d, 1H), |
| | 7.67-7.75 (m, 2H), 8.27 (dd, 1H), 8.33 (d, 1H). |
| Example 351 | 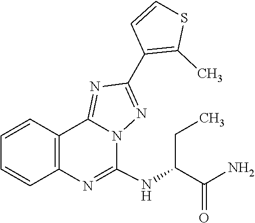 [SMILES: CC[C@@H](Nc1nc2ccccc2c2nc(-c3ccsc3C)nn12)C(N)=O] |
| | (2R)-2-{[2-(2-methylthiophen-3-yl[1,2,4]triazolo[1,5-c]quinazolin-5- |
| | yl]amino}butanamide |
| | LC-MS (Method 2): Rₜ = 1.25 min; MS (ESIpos): m/z = 367 [m + H]⁺ |
| | [α]²⁰_{D}: −68.6° (c = 1.00, DMSO) |
| | ¹H-NMR (400 MHz, DMSO-d₆): δ [ppm] = 0.95 (t, 3H), 1.87-1.99 (m, 1H), |
| | 2.01-2.14 (m, 1H), 2.94 (s, 3H), 4.70 (td, 1H), 7.32 (s, 1H), 7.41-7.47 (m, |
| | 2H), 7.49 (d, 1H), 7.63 (d, 1H), 7.69-7.75 (m, 3H), 8.28 (dd, 1H). |
| Example 352 | 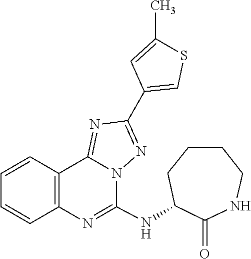 [SMILES: Cc1cc(-c2nc3c4ccccc4nc(N[C@@H]4CCCCNC4=O)n3n2)cs1] |
| | (3R)-3-{[2-(5-methylthiophen-3-yl)[1,2,4]triazolo[1,5-c]quinazolin-5- |
| | yl]amino}azepan-2-one |
| | LC-MS (Method 2): Rₜ = 1.34 min; MS (ESIpos): m/z = 393 [m + H]⁺ |
| | [α]²⁰_{D}: −66.9° (c = 1.00, DMSO) |
| | ¹H-NMR (400 MHz, DMSO-d₆): δ [ppm] = 1.25-1.38 (m, 1H), 1.48-1.61 (m, |
| | 1H), 1.81-1.95 (m, 2H), 1.97-2.07 (m, 1H), 2.27-2.36 (m, 1H), 2.55 (d, |
| | 3H), 3.11-3.21 (m, 1H), 3.32-3.41 (m, 1H and water signal), 4.83 (br dd, |
| | 1H), 7.44 (ddd, 1H), 7.51 (t, 1H), 7.63-7.68 (m, 2H), 7.73 (dd, 1H), 8.11 (d, |
| | 1H), 8.22 (dd, 1H), 8.26 (dd, 1H). |
| Example 353 | 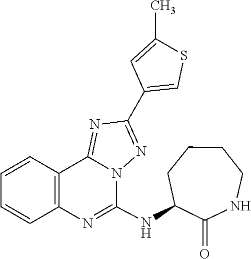 [SMILES: Cc1cc(-c2nc3c4ccccc4nc(N[C@H]4CCCCNC4=O)n3n2)cs1] |
| | (3S)-3-{[2-(5-methylthiophen-3-yl[1,2,4]triazolo[1,5-c]quinazolin-5- |
| | yl]amino}azepan-2-one |
| | LC-MS (Method 2): Rₜ = 1.35 min; MS (ESIpos): m/z = 393 [m + H]⁺ |
| | [α]²⁰_{D}: +70.4° (c = 1.00, DMSO) |
| | ¹H-NMR (400 MHz, DMSO-d₆): δ [ppm] = 1.26-1.39 (m, 1H), 1.49-1.61 (m, |
| | 1H), 1.80-1.95 (m, 2H), 1.97-2.07 (m, 1H), 2.28-2.35 (m, 1H), 2.55 (d, |
| | 3H), 3.10-3.21 (m, 1H), 3.31-3.43 (m, 1H and water signal), 4.83 (br dd, |
| | 1H), 7.42-7.47 (m, 1H), 7.51 (t, 1H), 7.64-7.68 (m, 2H), 7.71-7.76 (m, |
| | 1H), 8.11 (d, 1H), 8.22 (br dd, 1H), 8.26 (dd, 1H). |
| Example 354 | 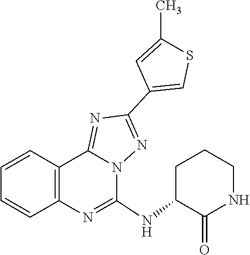 [SMILES: Cc1cc(-c2nc3c4ccccc4nc(N[C@@H]4CCCNC4=O)n3n2)cs1] |
| | (3R)-3-{[2-(5-methylthiophen-3-yl)[1,2,4]triazolo[1,5-c]quinazolin-5- |
| | yl]amino}piperidin-2-one |
| | LC-MS (Method 2): Rₜ = 1.21 min; MS (ESIpos): m/z = 379 [m + H]⁺ |
| | [α]²⁰_{D}: −25.3° (c = 1.00, DMSO) |
| | ¹H-NMR (400 MHz, DMSO-d₆): δ [ppm] = 1.83-1.98 (m, 2H), 2.07 (qd, 1H), |
| | 2.27-2.36 (m, 1H), 2.55 (d, 3H), 3.19-3.30 (m, 2H), 4.68 (dt, 1H), 7.40- |
| | 7.45 (m, 1H), 7.50 (t, 1H), 7.61-7.64 (m, 1H), 7.72 (ddd, 1H), 7.81 (br s, |
| | 1H), 8.06 (d, 1H), 8.08 (d, 1H), 8.25 (dd, 1H). |
| Example 355 | 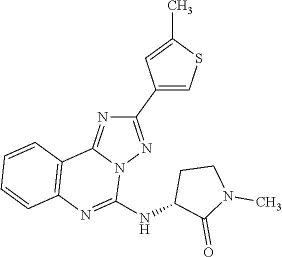 [SMILES: Cc1cc(-c2nc3c4ccccc4nc(N[C@@H]4CCN(C)C4=O)n3n2)cs1] |
| | (3R)-1-methyl-3-{[2-(5-methylthiophen-3-yl[1,2,4]triazolo[1,5- |
| | c]quinazolin-5-yl]amino}pyrrolidin-2-one |
| | LC-MS (Method 2): Rₜ = 1.20 min; MS (ESIpos): m/z = 379 [m + H]⁺ |
| | [α]²⁰_{D}: +9.9° (c = 1.00, DMSO) |
| | ¹H-NMR (400 MHz, DMSO-d₆): δ [ppm] = 2.21 (dq, 1H), 2.43-2.53 (m, 1H |
| | and DMSO signal), 2.55 (d, 3H), 2.83 (s, 3H), 3.44 (dd, 2H), 4.95 (q, 1H), |
| | 7.40-7.46 (m, 1H), 7.50 (s, 1H), 7.60 (d, 1H), 7.69-7.75 (m, 1H), 8.08 (d, |
| | 1H), 8.25 (dd, 1H), 8.34 (d, 1H). |
| Example 356 | 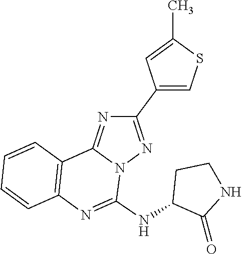 [SMILES: Cc1cc(-c2nc3c4ccccc4nc(N[C@@H]4CCNC4=O)n3n2)cs1] |
| | (3R)-3-{[2-(5-methylthiophen-3-yl)[1,2,4]triazolo[1,5-c]quinazolin-5- |
| | yl]amino}pyrrolidin-2-one |
| | LC-MS (Method 2): Rₜ = 1.13 min; MS (ESIpos): m/z = 365 [m + H]⁺ |
| | [α]²⁰_{D}: +5.6° (c = 1.00, DMSO) |
| | ¹H-NMR (400 MHz, DMSO-d₆): δ [ppm] = 2.24-2.36 (m, 1H), 2.55 (s, 3H), |
| | 4.88-4.96 (m, 1H), 7.40-7.45 (m, 1H), 7.51 (s, 1H), 7.62 (d, 1H), 7.69- |
| | 7.75 (m, 1H), 8.00 (s, 1H), 8.08 (d, 1H), 8.19 (d, 1H), 8.26 (dd, 1H). |
| Example 357 | 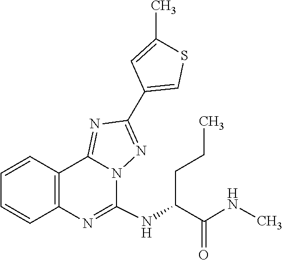 [SMILES: CCC[C@@H](Nc1nc2ccccc2c2nc(-c3csc(C)c3)nn12)C(=O)NC] |
| | N-methyl-N²-[2-(5-methylthiophen-3-yl)[1,2,4]triazolo[1,5-c]quinazolin-5- |
| | yl]-D-norvalinamide |
| | LC-MS (Method 2): Rₜ = 1.32 min; MS (ESIpos): m/z = 395 [m + H]⁺ |
| | [α]²⁰_{D}: −48.4° (c = 1.00, DMSO) |
| | ¹H-NMR (400 MHz, DMSO-d₆): δ [ppm] = 0.91 (t, 3H), 1.29-1.49 (m, 2H), |
| | 1.83-1.99 (m, 2H), 2.55 (s, 3H), 2.62 (d, 3H), 4.73-4.79 (m, 1H), 7.41- |
| | 7.46 (m, 1H), 7.54 (s, 1H), 7.59-7.65 (m, 2H), 7.69-7.74 (m, 1H), 8.10- |
| | 8.17 (m, 2H), 8.26 (dd, 1H). |
| Example 358 | 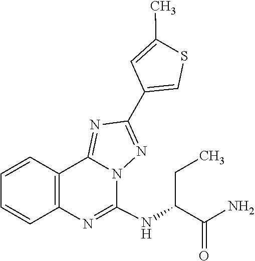 [SMILES: CC[C@@H](Nc1nc2ccccc2c2nc(-c3csc(C)c3)nn12)C(N)=O] |
| | (2R)-2-{[2-(5-methylthiophen-3-yl[1,2,4]triazolo[1,5-c]quinazolin-5- |
| | yl]amino}butanamide |
| | LC-MS (Method 2): Rₜ = 1.20 min; MS (ESIpos): m/z = 367 [m + H]⁺ |
| | [α]²⁰_{D}: −82.1° (c = 1.00, DMSO) |
| | ¹H-NMR (400 MHz, DMSO-d₆): δ [ppm] = 0.95 (t, 3H), 1.86-1.98 (m, 1H), |
| | 2.01-2.12 (m, 1H), 2.55 (d, 3H), 4.70 (td, 1H), 7.33 (s, 1H), 7.41-7.48 (m, |
| | 2H), 7.54 (t, 1H), 7.60-7.64 (m, 1H), 7.70-7.75 (m, 2H), 8.12 (d, 1H), 8.26 |
| | (dd, 1H). |

### Example 359

### (3R)-3-({2-[3,5-Bis(trifluoromethyl)phenyl][1,2,4]triazolo[1,5-c]quinazolin-5-yl}amino)azepan-2-one

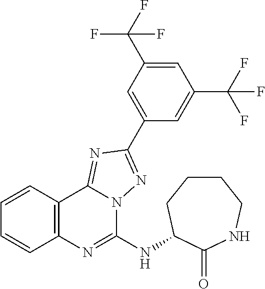 [SMILES: O=C1NCCCC[C@H]1Nc1nc2ccccc2c2nc(-c3cc(C(F)(F)F)cc(C(F)(F)F)c3)nn12]

2-[3,5-Bis(trifluoromethyl)phenyl]-5-chloro[1,2,4]triazolo[1,5-c]quinazoline (75.0 mg, 180 μmol) was suspended in DMSO (0.72 mL). (3R)-3-Aminoazepan-2-one (35 mg, 273 μmol) and N,N-diisopropylethylamine (63 μL, 360 μmol) were added. It was stirred at 60° C. and DMSO (0.5 mL) was added and the reaction mixture was stirred at 60° C. for 2 h. The reaction mixture was allowed to cool down, the solid material was filtered off, washed with DMSO (2×0.5 mL) and water (three times) and dried at 50° C. under vacuum affording 64 mg (70%) of the title compound.

LC-MS (Method 2): Rₜ=1.61 m; MS (ESIpos): m/z=509 [m+H]₊

[α]²⁰_{D}: +45.6° (c=1.00, pyridine)

¹H-NMR (400 MHz, Pyr): δ [ppm]=1.32-1.46 (m, 1H), 1.68-1.78 (m, 1H), 1.79-1.92 (m, 1H), 1.93-2.01 (m, 2H), 2.61 (br d, 1H), 3.29-3.47 (m, 2H), 5.18-5.24 (m, 1H), 7.45-7.51 (m, 1H), 7.75 (ddd, 1H), 7.98 (d, 1H), 8.22 (s, 1H), 8.59 (dd, 1H), 8.94 (s, 2H), 9.19 (t, 1H).

The following examples were prepared analogously to example 359 and the racemic mixtures were separated by chiral HPLC:

| | Structure |
|---|---|
| | IUPAC-Name |
| | LC-MS (method): Retention time; Mass found |
| Example | ¹H-NMR |
| Example 360 | 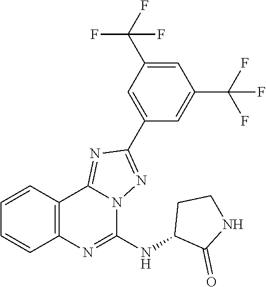 [SMILES: O=C1NCC[C@H]1Nc1nc2ccccc2c2nc(-c3cc(C(F)(F)F)cc(C(F)(F)F)c3)nn12] |
| | (3R)-3-({2-[3,5-bis(trifluoromethyl)phenyl][1,2,4]triazolo[1,5- |
| | c]quinazolin-5-yl}amino)pyrrolidin-2-one |
| | LC-MS (Method 2): Rₜ = 1.43 min; MS (ESIpos): m/z = 481 [m + H]⁺ |
| | [α]²⁰_{D}: +1.3° (c = 1.00, DMSO) |
| | ¹H-NMR (400 MHz, DMSO-d₆): δ [ppm] = 2.26-2.38 (m, 1H), 3.32-3.38 (m, |
| | 2H and water signal), 5.00 (q, 1H), 7.43-7.48 (m, 1H), 7.60-7.65 (m, 1H), |
| | 7.72-7.79 (m, 1H), 8.03 (s, 1H), 8.33-8.39 (m, 2H), 8.53 (d, 1H), 8.82 (s, |
| | 2H). |
| Example 361 | 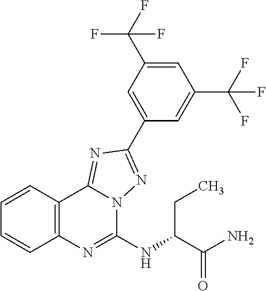 [SMILES: CC[C@@H](Nc1nc2ccccc2c2nc(-c3cc(C(F)(F)F)cc(C(F)(F)F)c3)nn12)C(N)=O] |
| | (2R)-2-({2-[3,5-bis(trifluoromethyl)phenyl][1,2,4]triazolo[1,5- |
| | c]quinazolin-5-yl}amino)butanamide |
| | LC-MS (Method 2): Rₜ = 1.47 min; MS (ESIpos): m/z = 483 [m + H]⁺ |
| | [α]²⁰_{D}: −56.1° (c = 1.00, DMSO) |
| | ¹H-NMR (400 MHz, DMSO-d₆): δ [ppm] = 0.99 (t, 3H), 1.90-2.13 (m, 2H), |
| | 4.70 (td, 1H), 7.29 (s, 1H), 7.42-7.47 (m, 1H), 7.59 (d, 1H), 7.68-7.75 (m, |
| | 2H), 8.00 (d, 1H), 8.32-8.37 (m, 2H), 8.81 (s, 2H). |
| Example 362 | 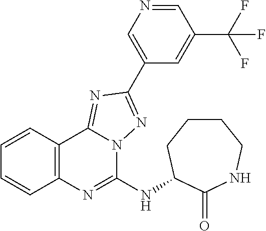 [SMILES: O=C1NCCCC[C@H]1Nc1nc2ccccc2c2nc(-c3cncc(C(F)(F)F)c3)nn12] |
| | (3R)-3-({2-[5-(trifluoromethyl)pyridin-3-yl][1,2,4]triazolo[1,5-c]quinazolin- |
| | 5-yl}amino)azepan-2-one |
| | LC-MS (Method 2): Rₜ = 1.35 min; MS (ESIpos): m/z = 442 [m + H]⁺ |
| | [α]²⁰_{D}: −60.2° (c = 1.00, DMSO) |
| | ¹H-NMR (400 MHz, DMSO-d₆): δ [ppm] = 1.26-1.40 (m, 1H), 1.55-1.67 (m, |
| | 1H), 1.81-1.95 (m, 2H), 1.98-2.08 (m, 1H), 2.27-2.35 (m, 1H), 3.13-3.22 |
| | (m, 1H), 3.30-3.42 (m, 1H and water signal), 4.86 (br dd, 1H), 7.48 (ddd, |
| | 1H), 7.67 (d, 1H), 7.76 (d, 1H), 7.85 (d, 1H), 8.20 (dd, 1H), 8.34 (dd, 1H), |
| | 8.83 (s, 1H), 9.20 (d, 1H), 9.68 (d, 1H). |
| Example 363 | 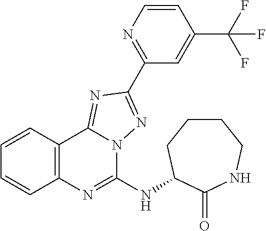 [SMILES: O=C1NCCCC[C@H]1Nc1nc2ccccc2c2nc(-c3cc(C(F)(F)F)ccn3)nn12] |
| | (3R)-3-({2-[4-(trifluoromethyl)pyridin-2-yl][1,2,4]triazolo[1,5-c]quinazolin- |
| | 5-yl}amino)azepan-2-one |
| | LC-MS (Method 2): Rₜ = 1.32 min; MS (ESIpos): m/z = 442 [m + H]⁺ |
| | [α]²⁰_{D}: −52.8° (c = 1.00, DMSO) |
| | ¹H-NMR (400 MHz, DMSO-d₆): δ [ppm] = 12.6-1.40 (m, 1H), 1.52-1.65 (m, |
| | 1H), 1.81-1.96 (m, 2H), 1.99-2.08 (m, 1H), 2.29-2.39 (m, 1H), 3.12-3.22 |
| | (m, 1H), 3.29-3.43 (m, 1H and water signal), 4.85 (br dd, 1H), 7.46-7.51 |
| | (m, 1H), 7.67-7.71 (m, 1H), 7.74-7.80 (m, 1H), 7.84 (d, 1H), 8.01 (dd, 1H), |
| | 8.24 (br dd, 1H), 8.36 (dd, 1H), 8.56 (s, 1H), 9.10 (d, 1H). |
| Example 364 | 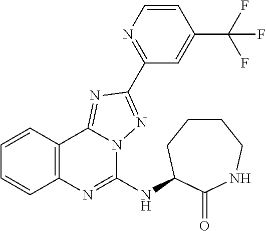 [SMILES: O=C1NCCCC[C@@H]1Nc1nc2ccccc2c2nc(-c3cc(C(F)(F)F)ccn3)nn12] |
| | (3S)-3-{2-[4-(trifluoromethyl)pyridin-2-yl][1,2,4]triazolo[1,5-c]quinazolin- |
| | 5-yl}amino)azepan-2-one |
| | LC-MS (Method 2): Rₜ = 1.30 min; MS (ESIpos): m/z = 442 [m + H]⁺ |
| | [α]²⁰_{D}: +6.3° (c = 1.00, DMSO) |
| | ¹H-NMR (400 MHz, DMSO-d₆): δ [ppm] = 1.27-1.40 (m, 1H), 1.51-1.64 (m, |
| | 1H), 1.80-1.96 (m, 2H), 1.98-2.08 (m, 1H), 2.29-2.37 (m, 1H), 3.12-3.22 |
| | (m, 1H), 4.85 (br dd, 1H), 7.46-7.51 (m, 1H), 7.67-7.71 (m, 1H), 7.74-7.80 |
| | (m, 1H), 7.84 (d, 1H), 8.01 (br d, 1H), 8.23 (br dd, 1H), 8.34-8.38 (m, 1H), |
| | 8.56 (s, 1H), 9.10 (d, 1H). |
| Example 365 | 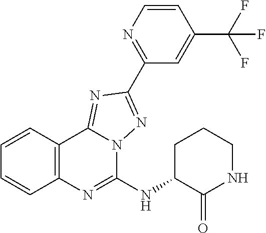 [SMILES: O=C1NCCC[C@H]1Nc1nc2ccccc2c2nc(-c3cc(C(F)(F)F)ccn3)nn12] |
| | (3R)-3-({2-[4-(trifluoromethyl)pyridin-2-yl][1,2,4]triazolo[1,5-c]quinazolin- |
| | 5-yl}amino)piperidin-2-one |
| | LC-MS (Method 2): Rₜ = 1.17 min; MS (ESIpos): m/z = 428 [m + H]⁺ |
| | [α]²⁰_{D}: −10.6° (c = 1.00, DMSO) |
| | ¹H-NMR (400 MHz, DMSO-d₆): δ [ppm] = 1.83-1.99 (m, 2H), 2.11 (qd, 1H), |
| | 2.24-2.34 (m, 1H), 3.19-3.31 (m, 2H), 4.72 (dt, 1H), 7.43-7.49 (m, 1H), |
| | 7.66 (d, 1H), 7.72-7.77 (m, 1H), 7.79 (br s, 1H), 8.00 (dd, 1H), 8.35 (dd, |
| | 1H), 8.38 (d, 1H), 8.59 (s, 1H), 9.09 (d, 1H). |
| Example 366 | 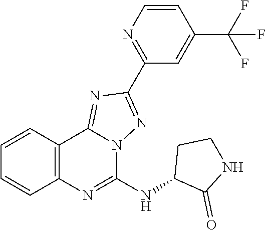 [SMILES: O=C1NCC[C@H]1Nc1nc2ccccc2c2nc(-c3cc(C(F)(F)F)ccn3)nn12] |
| | (3R)-3-({2-[4-(trifluoromethyl)pyridin-2-yl][1,2,4]triazolo[1,5-c]quinazolin- |
| | 5-yl}amino)pyrrolidin-2-one |
| | LC-MS (Method 2): Rₜ = 1.11 min; MS (ESIpos): m/z = 414 [m + H]⁺ |
| | [α]²⁰_{D}: +8.5° (c = 1.00, DMSO) |
| | ¹H-NMR (400 MHz, DMSO-d₆): δ [ppm] = 2.28-2.39 (m, 1H), 4.92-5.01 (m, |
| | 1H), 7.44-7.50 (m, 1H), 7.65 (d, 1H), 7.74-7.79 (m, 1H), 7.97-8.02 (m, |
| | 2H), 8.35 (dd, 1H), 8.54 (d, 1H), 8.60 (s, 1H), 9.09 (d, 1H). |
| Example 367 | 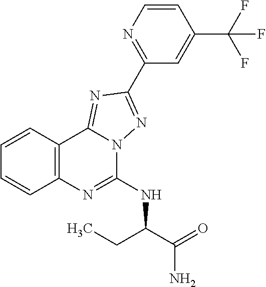 [SMILES: CC[C@@H](Nc1nc2ccccc2c2nc(-c3cc(C(F)(F)F)ccn3)nn12)C(N)=O] |
| | (2R)-2-({2-[4-(trifluoromethyl)pyridin-2-yl][1,2,4]triazolo[1,5-c]quinazolin- |
| | 5-yl}amino)butanamide |
| | LC-MS (Method 2): Rₜ = 1.16 min; MS (ESIpos): m/z = 416 [m + H]⁺ |
| | [α]²⁰_{D}: −53.9° (c = 1.00, DMSO) |
| | ¹H-NMR (400 MHz, DMSO-d₆): δ [ppm] = 0.96 (t, 3H), 1.90-2.02 (m, 1H), |
| | 2.03-2.15 (m, 1H), 4.70 (td, 1H), 7.32 (s, 1H), 7.45-7.50 (m, 1H), 7.65 (d, |
| | 1H), 7.70 (s, 1H), 7.73-7.80 (m, 2H), 8.01 (dd, 1H), 8.35 (dd, 1H), 8.60 (t, |
| | 1H), 9.10 (d, 1H). |
| Example 368 | 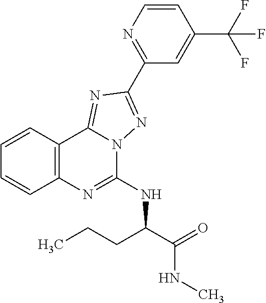 [SMILES: CCC[C@@H](Nc1nc2ccccc2c2nc(-c3cc(C(F)(F)F)ccn3)nn12)C(=O)NC] |
| | N-methyl-N²-{2-[4-(trifluoromethyl)pyridin-2-yl][1,2,4]triazolo[1,5- |
| | c]quinazolin-5-yl}-D-norvalinamide |
| | LC-MS (Method 2): Rₜ = 1.26 min; MS (ESIpos): m/z = 444 [m + H]⁺ |
| | [α]²⁰_{D}: −51.3° (c = 1.00, DMSO) |
| | ¹H-NMR (400 MHz, DMSO-d₆): δ [ppm] = 0.92 (t, 3H), 1.31-1.51 (m, 2H), |
| | 1.87-2.02 (m, 2H), 2.62 (d, 3H), 4.78 (td, 1H), 7.47 (ddd, 1H), 7.64 (d, 1H), |
| | 7.75 (ddd, 1H), 7.97 (d, 1H), 8.01 (dd, 1H), 8.11 (q, 1H), 8.35 (dd, 1H), 8.61 |
| | (t, 1H), 9.10 (d, 1H). |
| Example 369 | 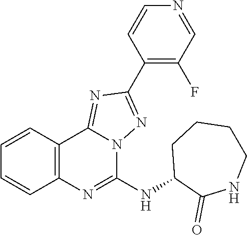 [SMILES: O=C1NCCCC[C@H]1Nc1nc2ccccc2c2nc(-c3ccncc3F)nn12] |
| | (3R)-3-{[2-(3-fluoropyridin-4-yl)[1,2,4]triazolo[1,5-c]quinazolin-5- |
| | yl]amino}azepan-2-one |
| | LC-MS (Method 2): Rₜ = 1.15 min; MS (ESIpos): m/z = 392 [m + H]⁺ |
| | [α]²⁰_{D}: −74.2° (c = 1.00, DMSO) |
| | ¹H-NMR (400 MHz, DMSO-d₆): δ [ppm] = 1.26-1.39 (m, 1H), 1.50-1.62 (m, |
| | 1H), 1.81-1.96 (m, 2H), 1.98-2.08 (m, 1H), 2.30-2.38 (m, 1H), 3.12-3.21 |
| | (m, 1H), 3.31-3.41 (m, 1H and water signal), 4.83 (br dd, 1H), 7.48 (ddd, |
| | 1H), 7.68-7.71 (m, 1H), 7.75-7.80 (m, 2H), 8.21-8.28 (m, 2H), 8.32 (dd, |
| | 1H), 8.67 (dd, 1H), 8.85 (d, 1H). |
| Example 370 | 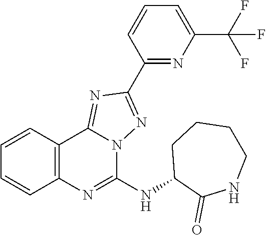 [SMILES: O=C1NCCCC[C@H]1Nc1nc2ccccc2c2nc(-c3cccc(C(F)(F)F)n3)nn12] |
| | (3R)-3-({2-[6-(trifluoromethyl)pyridin-2-yl][1,2,4]triazolo[1,5-c]quinazolin- |
| | 5-yl}amino)azepan-2-one |
| | LC-MS (Method 2): Rₜ = 1.30 min; MS (ESIpos): m/z = 442 [m + H]⁺ |
| | [α]²⁰_{D}: −70.6° (c = 1.00, DMSO) |
| | ¹H-NMR (400 MHz, DMOS-d₆): δ [ppm] = 1.27-1.39 (m, 1H), 1.53-1.64 (m, |
| | 1H), 1.81-1.96 (m, 2H), 1.99-2.08 (m, 1H), 2.29-2.37 (m, 1H), 3.12-3.22 |
| | (m, 1H), 3.31-3.42 (m, 1H and water signal), 4.86 (br dd, 1H), 7.49 (ddd, |
| | 1H), 7.69-7.72 (m, 1H), 7.78 (ddd, 1H), 7.81 (d, 1H), 8.12 (dd, 1H), 8.23 |
| | (dd, 1H), 8.34-8.39 (m, 2H), 8.66 (d, 1H). |
| Example 371 | 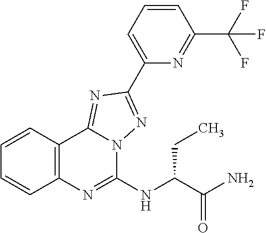 [SMILES: CC[C@@H](Nc1nc2ccccc2c2nc(-c3cccc(C(F)(F)F)n3)nn12)C(N)=O] |
| | (2R)-2-({2-[6-(trifluoromethyl)pyridin-2-yl][1,2,4]triazolo[1,5-c]quinazolin- |
| | 5-yl}amino)butanamide |
| | LC-MS (Method 2): Rₜ = 1.15 min; MS (ESIpos): m/z = 416 [m + H]⁺ |
| | [α]²⁰_{D}: −51.1° (c = 1.00, DMSO) |
| | ¹H-NMR (400 MHz, DMSO-d₆): δ [ppm] = 0.97 (t, 3H), 1.91-2.02 (m, 1H), |
| | 2.04-2.15 (m, 1H), 4.71 (td, 1H), 7.32 (s, 1H), 7.48 (ddd, 1H), 7.66 (d, 1H), |
| | 7.71 (s, 1H), 7.73-7.79 (m, 2H), 8.12 (dd, 1H), 8.34-8.39 (m, 2H), 8.69 (d, |
| | 1H). |
| Example 372 | 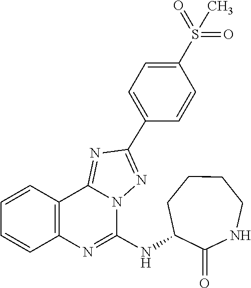 [SMILES: CS(=O)(=O)c1ccc(-c2nc3c4ccccc4nc(N[C@@H]4CCCCNC4=O)n3n2)cc1] |
| | (3R)-3-({2-[4-(methanesulfonyl)phenyl][1,2,4]triazolo[1,5-c]quinazolin-5- |
| | yl}amino)azepan-2-one |
| | LC-MS (Method 2): Rₜ = 1.17 min; MS (ESIpos): m/z = 451 [m + H]⁺ |
| | [α]²⁰_{D}: −71.8° (c = 1.00, DMSO) |
| | ¹H-NMR (400 MHz, DMSO-d₆): δ [ppm] = 1.26-1.39 (m, 1H), 1.52-1.64 (m, |
| | 1H), 1.81-1.96 (m, 2H), 1.99-2.08 (m, 1H), 2.29-2.37 (m, 1H), 3.12-3.22 |
| | (m, 1H), 3.32 (s, 3H), 3.33-3.44 (m, 1H and water signal), 4.85 (br dd, 1H), |
| | 7.45-7.50 (m, 1H), 7.67-7.70 (m, 1H), 7.74-7.81 (m, 2H), 8.16 (d, 2H), |
| | 8.23 (br dd, 1H), 8.33 (dd, 1H), 8.53 (d, 2H). |
| Example 373 | 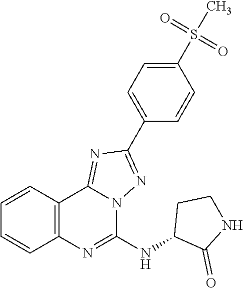 [SMILES: CS(=O)(=O)c1ccc(-c2nc3c4ccccc4nc(N[C@@H]4CCNC4=O)n3n2)cc1] |
| | (3R)-3-({2-[4-(methanesulfonyl)phenyl][1,2,4]triazolo[1,5-c]quinazolin-5- |
| | yl}amino)pyrrolidin-2-one |
| | LC-MS (Method 2): Rₜ = 0.96 min; MS (ESIpos): m/z = 423 [m + H]⁺ |
| | [α]²⁰_{D}: +5.2° (c = 1.00, DMSO) |
| | ¹H-NMR (400 MHz, DMSO-d₆): δ [ppm] = 2.27-2.39 (m, 1H), 3.32 (s, 3H), |
| | 3.33-3.38 (m, 2H and water signal), 4.92-5.00 (m, 1H), 7.44-7.48 (m, 1H), |
| | 7.64 (d, 1H), 7.75 (ddd, 1H), 8.01 (s, 1H), 8.16-8.20 (m, 2H), 8.32 (dd, 1H), |
| | 8.42 (d, 1H), 8.53-8.57 (m, 2H). |
| Example 374 | 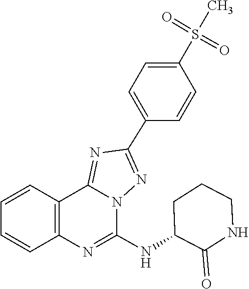 [SMILES: CS(=O)(=O)c1ccc(-c2nc3c4ccccc4nc(N[C@@H]4CCCNC4=O)n3n2)cc1] |
| | (3R)-3-({2-[4-(methanesulfonyl)phenyl][1,2,4]triazolo[1,5-c]quinazolin-5- |
| | yl}amino)piperidin-2-one |
| | LC-MS (Method 2): Rₜ = 1.03 min; MS (ESIpos): m/z = 436 [m + H]⁺ |
| | [α]²⁰_{D}: −15.5° (c = 1.00, DMSO) |
| | ¹H-NMR (400 MHz, DMSO-d₆): δ [ppm] = 1.85-1.99 (m, 2H), 2.11 (qd, 1H), |
| | 2.26-2.35 (m, 1H), 3.20-3.31 (m, 2H), 3.32 (s, 3H), 4.72 (dt, 1H), 7.45 |
| | (ddd, 1H), 7.65 (d, 1H), 7.74 (ddd, 1H), 7.82 (br s, 1H), 8.15-8.20 (m, 2H), |
| | 8.29 (d, 1H), 8.32 (dd, 1H), 8.51-8.56 (m, 2H). |
| Example 375 | 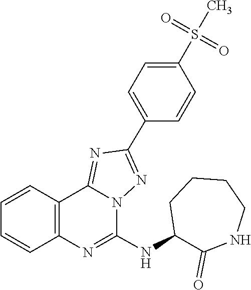 [SMILES: CS(=O)(=O)c1ccc(-c2nc3c4ccccc4nc(N[C@H]4CCCCNC4=O)n3n2)cc1] |
| | (3S)-3-({2-[4-(methanesulfonyl)phenyl][1,2,4]triazolo[1,5-c]quinazolin-5- |
| | yl}amino)azepan-2-one |
| | LC-MS (Method 2): Rₜ = 1.17 min; MS (ESIpos): m/z = 451 [m + H]⁺ |
| | [α]²⁰_{D}: +76.0° (c = 1.00, DMSO) |
| | ¹H-NMR (400 MHz, DMSO-d₆): δ [ppm] = 1.26-1.40 (m, 1H), 1.52-1.65 (m, |
| | 1H), 1.81-1.96 (m, 2H), 1.98-2.08 (m, 1H), 2.29-2.37 (m, 1H), 3.12-3.22 |
| | (m, 1H), 3.32 (s, 3H), 4.85 (br dd, 1H), 7.45-7.50 (m, 1H), 7.67-7.70 (m, |
| | 1H), 7.74-7.81 (m, 2H), 8.16 (d, 2H), 8.22 (br dd, 1H), 8.33 (dd, 1H), 8.53 |
| | (d, 2H). |
| Example 376 | 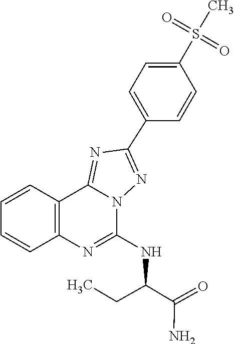 [SMILES: CC[C@@H](Nc1nc2ccccc2c2nc(-c3ccc(S(C)(=O)=O)cc3)nn12)C(N)=O] |
| | (2R)-2-({2-[4-(methanesulfonyl)phenyl][1,2,4]triazolo[1,5-c]quinazolin-5- |
| | yl}amino)butanamide |
| | LC-MS (Method 2): Rₜ = 1.01 min; MS (ESIpos): m/z = 425 [m + H]⁺ |
| | [α]²⁰_{D}: −81.1° (c = 1.00, DMSO) |
| | ¹H-NMR (400 MHz, DMSO-d₆): δ [ppm] = 0.97 (t, 3H), 1.89-2.01 (m, 1H), |
| | 2.03-2.14 (m, 1H), 3.32 (s, 3H), 4.70 (td, 1H), 7.33 (s, 1H), 7.47 (ddd, 1H), |
| | 7.64 (d, 1H), 7.69-7.77 (m, 3H), 8.15-8.18 (m, 2H), 8.32 (dd, 1H), 8.54- |
| | 8.58 (m, 2H). |
| Example 377 | 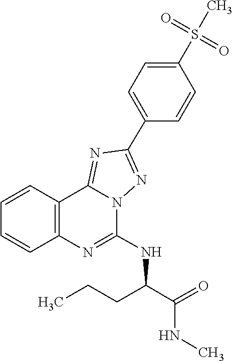 [SMILES: CCC[C@@H](Nc1nc2ccccc2c2nc(-c3ccc(S(C)(=O)=O)cc3)nn12)C(=O)NC] |
| | N²-{2-[4-(methanesulfonyl)phenyl][1,2,4]triazolo[1,5-c]quinazolin-5-yl}- |
| | N-methyl-D-norvalinamide |
| | LC-MS (Method 2): Rₜ = 1.13 min; MS (ESIpos): m/z = 453 [m + H]⁺ |
| | [α]²⁰_{D}: −53.3° (c = 1.00, DMSO) |
| | ¹H-NMR (400 MHz, DMSO-d₆): δ [ppm] = 0.92 (t, 3H), 1.31-1.52 (m, 2H), |
| | 1.86-2.01 (m, 2H), 2.63 (d, 3H), 3.32 (s, 3H), 4.77 (td, 1H), 7.46 (ddd, 1H), |
| | 7.64 (d, 1H), 7.74 (ddd, 1H), 7.88 (d, 1H), 8.13 (q, 1H), 8.15-8.19 (m, 2H), |
| | 8.32 (dd, 1H), 8.55-8.59 (m, 2H). |
| Example 378 | 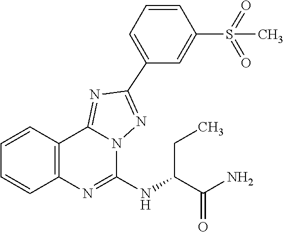 [SMILES: CC[C@@H](Nc1nc2ccccc2c2nc(-c3cccc(S(C)(=O)=O)c3)nn12)C(N)=O] |
| | (2R)-2-({2-[3-(methanesulfonyl)phenyl][1,2,4]triazolo[1,5-c]quinazolin-5- |
| | yl}amino)butanamide |
| | LC-MS (Method 1): Rₜ = 1.01 min; MS (ESIpos): m/z = 425 [m + H]⁺ |
| | [α]²⁰_{D}: −62.0° (c = 1.00, DMSO) |
| | ¹H-NMR (400 MHz, DMSO-d₆): δ [ppm] = 0.97 (t, 3H), 1.89-2.02 (m, 1H), |
| | 2.03-2.14 (m, 1H), 3.35 (s, 3H), 4.70 (td, 1H), 7.31 (s, 1H), 7.47 (ddd, 1H), |
| | 7.65 (d, 1H), 7.70 (s, 1H), 7.73-7.78 (m, 2H), 7.91 (t, 1H), 8.13 (ddd, 1H), |
| | 8.34 (dd, 1H), 8.66 (dt, 1H), 8.81 (t, 1H). |
| Example 379 | 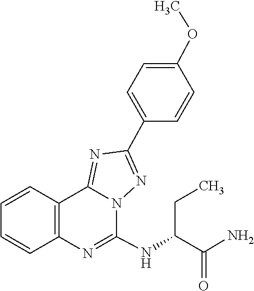 [SMILES: CC[C@@H](Nc1nc2ccccc2c2nc(-c3ccc(OC)cc3)nn12)C(N)=O] |
| | (2R)-2-{[2-(4-methoxyphenyl)[1,2,4]triazolo[1,5-c]quinazolin-5- |
| | yl]amino}butanamide |
| | LC-MS (Method 2): Rₜ = 1.16 min; MS (ESIpos): m/z = 377 [m + H]⁺ |
| | [α]²⁰_{D}: −78.0° (c = 1.00, DMSO) |
| | ¹H-NMR (400 MHz, DMSO-d₆): δ [ppm] = 0.96 (t, 3H), 1.87-2.00 (m, 1H), |
| | 2.01-2.13 (m, 1H), 3.86 (s, 3H), 4.70 (td, 1H), 7.12-7.17 (m, 2H), 7.32 (s, |
| | 1H), 7.41-7.46 (m, 1H), 7.54 (d, 1H), 7.62 (d, 1H), 7.69-7.75 (m, 2H), 8.23- |
| | 8.30 (m, 3H). |
| Example 381 | 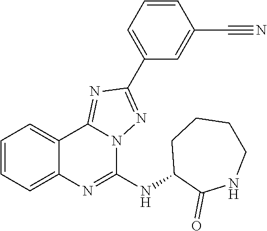 [SMILES: N#Cc1cccc(-c2nc3c4ccccc4nc(N[C@@H]4CCCCNC4=O)n3n2)c1] |
| | 3-(5-{[(3R)-2-oxoazepan-3-yl]amino}[1,2,4]triazolo[1,5-c]quinazolin-2- |
| | yl)benzonitrile |
| | LC-MS (Method 1): Rₜ = 1.30 min; MS (ESIpos): m/z = 398 [m + H]⁺ |
| | [α]²⁰_{D}: −103.6° (c = 1.00, DMSO) |
| | ¹H-NMR (400 MHz, DMSO-d₆): δ [ppm] = 1.25-1.39 (m, 1H), 1.53-1.64 (m, |
| | 1H), 1.82-1.95 (m, 2H), 1.97-2.08 (m, 1H), 2.28-2.35 (m, 1H), 3.12-3.21 |
| | (m, 1H), 3.31-3.42 (m, 1H and water signal), 4.85 (ddd, 1H), 7.48 (ddd, 1H), |
| | 7.67-7.70 (m, 1H), 7.77 (ddd, 1H), 7.79-7.86 (m, 2H), 8.06 (dt, 1H), 8.22 |
| | (dd, 1H), 8.32 (dd, 1H), 8.59 (dt, 1H), 8.61-8.63 (m, 1H). |
| Example 382 | 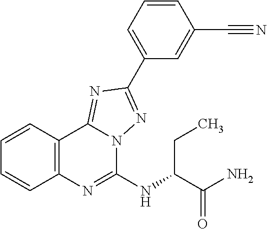 [SMILES: CC[C@@H](Nc1nc2ccccc2c2nc(-c3cccc(C#N)c3)nn12)C(N)=O] |
| | (2R)-2-{[2-(3-cyanophenyl)[1,2,4]triazolo[1,5-c]quinazolin-5- |
| | yl]amino}butanamide |
| | LC-MS (Method 1): Rₜ = 1.16 min; MS (ESIpos): m/z = 372 [m + H]⁺ |
| | [α]²⁰_{D}: −74.7° (c = 1.00, DMSO) |
| | ¹H-NMR (400 MHz, DMSO-d₆): δ [ppm] = 0.98 (t, 3H), 1.88-2.01 (m, 1H), |
| | 2.02-2.13 (m, 1H), 4.70 (td, 1H), 7.31 (s, 1H), 7.46 (ddd, 1H), 7.64 (d, 1H), |
| | 7.69-7.72 (m, 2H), 7.74 (ddd, 1H), 7.83 (dtt, 1H), 8.05 (dt, 1H), 8.31 (dd, |
| | 1H), 8.61 (dt, 1H), 8.65-8.67 (m, 1H). |
| Example 383 | 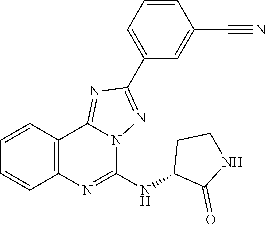 [SMILES: N#Cc1cccc(-c2nc3c4ccccc4nc(N[C@@H]4CCNC4=O)n3n2)c1] |
| | 3-(5-{[(3R)-2-oxopyrrolidin-3-yl]amino}[1,2,4]triazolo[1,5-c]quinazolin-2- |
| | yl)benzonitrile |
| | LC-MS (Method 1): Rₜ = 1.11 min; MS (ESIpos): m/z = 370 [m + H]⁺ |
| | [α]²⁰_{D}: +7.2° (c = 1.00, DMSO) |
| | ¹H-NMR (400 MHz, DMSO-d₆): δ [ppm] = 2.25-2.38 (m, 1H), 2.50-2.56 (m, |
| | 1H and DMSO signal), 3.34-3.37 (m, 1H), 4.93-5.01 (m, 1H), 7.45 (ddd, |
| | 1H), 7.63 (d, 1H), 7.72-7.77 (m, 1H), 7.81-7.86 (m, 1H), 8.01-8.07 (m, |
| | 2H), 8.30 (dd, 1H), 8.39 (br d, 1H), 8.56-8.60 (m, 1H), 8.61-8.63 (m, 1H). |
| Example 384 | 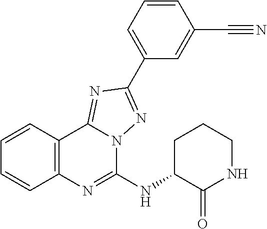 [SMILES: N#Cc1cccc(-c2nc3c4ccccc4nc(N[C@@H]4CCCNC4=O)n3n2)c1] |
| | 3-(5-{[(3R)-2-oxopiperidin-3-yl]amino}[1,2,4]triazolo[1,5-c]quinazolin-2- |
| | yl)benzonitrile |
| | LC-MS (Method 1): Rₜ = 1.17 min; MS (ESIpos): m/z = 384 [m + H]⁺ |
| | [α]²⁰_{D}: −12.5° (c = 1.00, DMSO) |
| | 1H-NMR (400 MHz, DMSO-d₆): Shift [ppm] = 1.87-1.98 (m, 2H), 2.10 (qd, |
| | 1H), 2.25-2.34 (m, 1H), 3.20-3.30 (m, 2H), 4.69-4.77 (m, 1H), 7.43-7.48 |
| | (m, 1 H), 7.65 (d, 1H), 7.72-7.77 (m, 1H), 7.81-7.86 (m, 2H), 8.03-8.07 (m, |
| | 1H), 8.27 (d, 1H), 8.29-8.32 (m, 1H), 8.56-8.60 (m, 1H), 8.61-8.63 (m, |
| | 1H). |
| Example 385 | 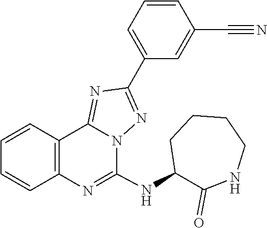 [SMILES: N#Cc1cccc(-c2nc3c4ccccc4nc(N[C@H]4CCCCNC4=O)n3n2)c1] |
| | 3-(5-{[(3S)-2-oxoazepan-3-yl]amino}[1,2,4]triazolo[1,5-c]quinazolin-2- |
| | yl)benzonitrile |
| | LC-MS (Method 1): Rₜ = 1.32 min; MS (ESIpos): m/z = 398 [m + H]⁺ |
| | ¹H-NMR (400 MHz, DMSO-d₆): δ [ppm] = 1.26-1.39 (m, 1H), 1.53-1.65 (m, |
| | 1H), 1.81-1.96 (m, 2H), 1.98-2.08 (m, 1H), 2.28-2.35 (m, 1H), 3.12-3.22 |
| | (m, 1H), 3.31-3.42 (m, 1H and water signal), 4.83-4.88 (m, 1H), 7.45-7.51 |
| | (m, 1H), 7.67-7.70 (m, 1H), 7.73-7.86 (m, 3H), 8.06 (dt, 1H), 8.20-8.24 |
| | (m, 1H), 8.32 (dd, 1H), 8.59 (d, 1H), 8.62 (s, 1H). |
| Example 386 | 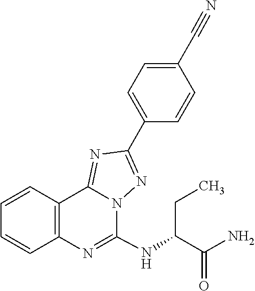 [SMILES: CC[C@@H](Nc1nc2ccccc2c2nc(-c3ccc(C#N)cc3)nn12)C(N)=O] |
| | (2R)-2-{[2-(4-cyanophenyl)[1,2,4]triazolo[1,5-c]quinazolin-5- |
| | yl]amino}butanamide |
| | LC-MS (Method 1): Rₜ = 1.14 min; MS (ESIpos): m/z = 372 [m + H]⁺ |
| | [α]²⁰_{D}: −81.5° (c = 1.00, DMSO) |
| | ¹H-NMR (400 MHz, DMSO-d₆): δ [ppm] = 0.97 (t, 3H), 1.88-2.00 (m, 1H), |
| | 2.02-2.13 (m, 1H), 4.70 (td, 1H), 7.33 (s, 1H), 7.46 (ddd, 1H), 7.63 (d, 1H), |
| | 7.69 (d, 1H), 7.71-7.77 (m, 2H), 8.04-8.08 (m, 2H), 8.29 (dd, 1H), 8.44- |
| | 8.48 (m, 2H). |
| Example 387 | 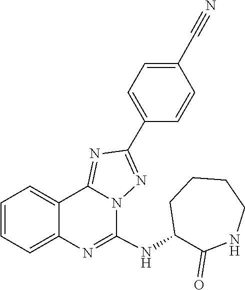 [SMILES: N#Cc1ccc(-c2nc3c4ccccc4nc(N[C@@H]4CCCCNC4=O)n3n2)cc1] |
| | 4-(5-{[(3R)-2-oxoazepan-3-yl]amino}[1,2,4]triazolo[1,5-c]quinazolin-2- |
| | yl)benzonitrile |
| | LC-MS (Method 1): Rₜ = 1.32 min; MS (ESIpos): m/z = 398 [m + H]⁺ |
| | [α]²⁰_{D}: −45.1° (c = 1.00, DMSO) |
| | ¹H-NMR (400 MHz, DMSO-d₆): δ [ppm] = 1.26-1.39 (m, 1H), 1.51-1.63 (m, |
| | 1H), 1.81-1.95 (m, 2H), 1.98-2.08 (m, 1H), 2.28-2.37 (m, 1H), 3.12-3.21 |
| | (m, 1H), 3.31-3.41 (m, 1H and water signal), 4.84 (br dd, 1H), 7.47 (ddd, |
| | 1H), 7.66-7.70 (m, 1H), 7.74-7.80 (m, 2H), 8.05-8.09 (m, 2H), 8.23 (dd, |
| | 1H), 8.31 (dd, 1H), 8.43-8.46 (m, 2H). |
| Example 388 | 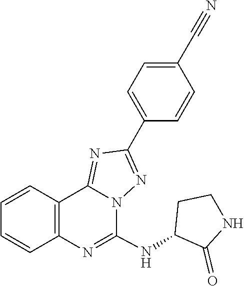 [SMILES: N#Cc1ccc(-c2nc3c4ccccc4nc(N[C@@H]4CCNC4=O)n3n2)cc1] |
| | 4-(5-{[(3R)-2-oxopyrrolidin-3-yl]amino}[1,2,4]triazolo[1,5-c]quinazolin-2- |
| | yl)benzonitrile |
| | LC-MS (Method 1): Rₜ = 1.10 min; MS (ESIpos): m/z = 370 [m + H]⁺ |
| | [α]²⁰_{D}: +4.2° (c = 1.00, DMSO) |
| | ¹H-NMR (400 MHz, DMSO-d₆): δ [ppm] = 2.26-2.37 (m, 1H), 3.34-3.37 (m, |
| | 1H), 4.95 (q, 1H), 7.45 (ddd, 1H), 7.62-7.65 (m, 1H), 7.75 (ddd, 1H), 8.02 |
| | (s, 1H), 8.07-8.10 (m, 2H), 8.28-8.32 (m, 1H), 8.39 (br d, 1H), 8.43-8.47 |
| | (m, 2H). |
| Example 389 | 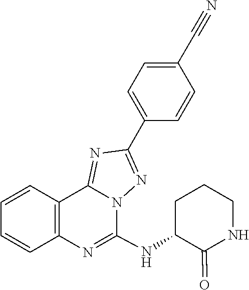 [SMILES: N#Cc1ccc(-c2nc3c4ccccc4nc(N[C@@H]4CCCNC4=O)n3n2)cc1] |
| | 4-(5-{[(3R)-2-oxopiperidin-3-yl]amino}[1,2,4]triazolo[1,5-c]quinazolin-2- |
| | yl)benzonitrile |
| | LC-MS (Method 1): Rₜ = 1.17 min; MS (ESIpos): m/z = 384 [m + H]⁺ |
| | [α]²⁰_{D}: −332.6° (c = 1.00, DMSO) |
| | ¹H-NMR (400 MHz, DMSO-d₆): δ [ppm] = 1.85-1.99 (m, 2H), 2.10 (dq, 1H), |
| | 2.26-2.34 (m, 1H), 3.20-3.30 (m, 2H), 4.67-4.76 (m, 1H), 7.42-7.47 (m, |
| | 1H), 7.64 (d, 1H), 7.71-7.76 (m, 1H), 7.83 (br s, 1H), 8.04-8.09 (m, 2H), |
| | 8.24-8.31 (m, 2H), 8.40-8.46 (m, 2H). |

### Example 390

### (3R)-3-{[2-(Imidazo[1,2-a]pyridin-7-yl)[1,2,4]triazolo[1,5-c]quinazolin-5-yl]amino}azepan-2-one

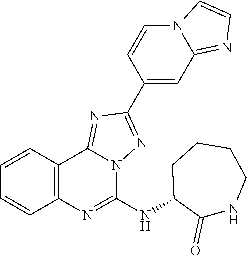 [SMILES: O=C1NCCCC[C@H]1Nc1nc2ccccc2c2nc(-c3ccn4ccnc4c3)nn12]

5-Chloro-2-(imidazo[1,2-a]pyridin-7-yl)[1,2,4]triazolo[1,5-c]quinazoline (85.0 mg, 265 μmol) was suspended in DMSO (2.00 mL). (3R)-3-Aminoazepan-2-one (51 mg, 398 μmol) and N,N-diisopropylethylamine (92 μL, 530 μmol) were added. It was stirred at 60° C. for 7 h. The reaction mixture was allowed to cool down and the precipitate was filtered off. The filtrate and the solid material were combined and DMSO (2.00 mL) was added. The solid material was dissolved in the heat and allowed to cool down slowly. The precipitate was filtered off, washed twice with DMSO and five times with water. The solid material in DMSO (1.5 mL) was dissolved in the heat and allowed to cool down to rt. The precipitate was filtered off, washed twice with DMSO and five times with water, dried under vacuum at 50° C. and purified by HPLC to afford 22 mg (20%) of the title compound.

LC-MS (Method 2): Rₜ=1.08 min; MS (ESIpos): m/z=413 [m+H]⁺

[α]²⁰_{D}: +107.7° (c=1.00, DMSO)

¹H-NMR (400 MHz, DMSO-d₆): δ [ppm]=1.27-1.40 (m, 1H), 1.53-1.65 (m, 1H), 1.82-1.96 (m, 2H), 1.99-2.08 (m, 1H), 2.29-2.37 (m, 1H), 3.13-3.22 (m, 1H), 3.31-3.42 (m, 1H and water signal), 4.85 (br dd, 1H), 7.48 (ddd, 1H), 7.67-7.70 (m, 1H), 7.71-7.79 (m, 3H), 7.80 (d, 1H), 8.12-8.14 (m, 1H), 8.22 (dd, 1H), 8.34 (dd, 1H), 8.40-8.42 (m, 1H), 8.75 (dd, 1H).

### Example 391

### N²-[2-(4-Methoxyphenyl)[1,2,4]triazolo[1,5-c]quinazolin-5-yl]-N-propan-2-yl-D-alaninamide

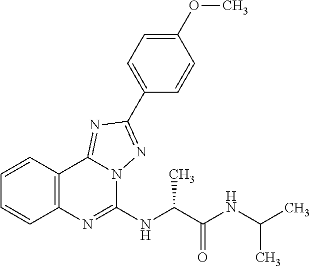 [SMILES: COc1ccc(-c2nc3c4ccccc4nc(N[C@H](C)C(=O)NC(C)C)n3n2)cc1]

Step 1: 5-Chloro-2-(4-methoxyphenyl)[1,2,4]triazolo[1,5-c]quinazoline (983.0 mg, 3.16 mmol) and (2R)-1-methoxy-1-oxopropan-2-aminium chloride (662 mg, 4.74 mmol) were suspended in DMSO (13.0 mL). N,N-Diisopropylethylamine (1.6 mL, 9.30 mmol) was added and it was stirred at 60° C. for 1.5 h. The reaction mixture was allowed to reach rt and stirred at rt overnight. Water (130 mL) was added and the precipitate was filtered off, washed twice with water and once with 2-methoxy-2-methylpropane, and dried under vacuum at 50° C. to obtain 750 mg (64%) of methyl N-[2-(4-methoxyphenyl)[1,2,4]triazolo[1,5-c]quinazolin-5-yl]-D-alaninate.

LC-MS (Method 2): Rₜ=1.35 min; MS (ESIpos): m/z=378 [m+H]⁺

[α]²⁰_{D}: −6.9° (c=1.00, Ethanol)

¹H-NMR (400 MHz, DMSO-d₆): δ [ppm]=1.61 (d, 3H), 3.69 (s, 3H), 3.86 (s, 3H), 4.90 (quin, 1H), 7.13-7.18 (m, 2H), 7.45 (ddd, 1H), 7.60 (d, 1H), 7.72 (ddd, 1H), 8.24-8.31 (m, 3H), 8.40 (d, 1H).

Step 2: Methyl N-[2-(4-methoxyphenyl)[1,2,4]triazolo[1,5-c]quinazolin-5-yl]-D-alaninate (566.0 mg, 1.50 mmol) was suspended in methanol (5.4 mL). 2 M NaOH (2.7 mL) was added and it was stirred for 1 h at rt. The reaction mixture was poured into water (50 mL) and acidified with 2 M HCl to pH 2. The precipitate was filtered off, washed three times with water and dried under vacuum at 50° C. to yield 303 mg (56%) of N-[2-(4-methoxyphenyl)[1,2,4]triazolo[1,5-c]quinazolin-5-yl]-D-alanine.

LC-MS (Method 2): Rₜ=0.71 min; MS (ESIpos): m/z=364 [m+H]⁺

1H-NMR (400 MHz, DMSO-d₆): δ [ppm]=1.61 (d, 3H), 3.86 (s, 3H), 4.80 (quin, 1H), 7.12-7.18 (m, 2H), 7.44 (ddd, 1H), 7.60-7.63 (m, 1H), 7.72 (ddd, 1H), 8.15 (d, 1H), 8.23-8.31 (m, 3H), 12.86 (br s, 1H).

Step 3: N-[2-(4-Methoxyphenyl)[1,2,4]triazolo[1,5-c]quinazolin-5-yl]-D-alanine (101 mg, 278 μmol) was dissolved in DMF (2.5 mL). HATU (143 mg, 375 μmol) and 4-methylmorpholine (82 μL, 750 μmol) were added. It was stirred for 15 minutes and then isopropylamine (64 μL, 750 μmol) was added and stirring at rt was continued for 1 h before isopropylamine (130 μL, 1500 μmol) was added. It was stirred overnight at rt. The reaction mixture was purified by HPLC obtaining 18 mg (16%) of the title compound.

LC-MS (Method 2): Rₜ=1.27 min; MS (ESIpos): m/z=405 [m+H]⁺

[α]²⁰_{D}: +2.1° (c=1.00, DMSO)

¹H-NMR (400 MHz, DMSO-d₆): δ [ppm]=1.09 (t, 6H), 1.51 (d, 3H), 3.83-3.95 (m, 4H), 4.71 (quin, 1H), 7.12-7.17 (m, 2H), 7.44 (ddd, 1H), 7.62 (t, 2H), 7.72 (ddd, 1H), 8.07 (d, 1H), 8.23-8.27 (m, 2H), 8.29 (dd, 1H).

### Example 392

### (3R)-3-{[2-(4-Methoxyphenyl)-9-(propan-2-yl)[1,2,4]triazolo[1,5-c]quinazolin-5-yl]amino}azepan-2-one

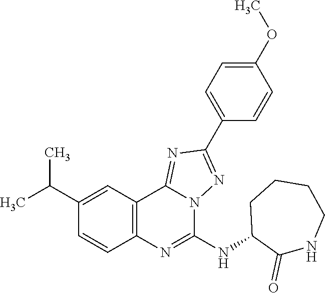 [SMILES: COc1ccc(-c2nc3c4cc(C(C)C)ccc4nc(N[C@@H]4CCCCNC4=O)n3n2)cc1]

(3R)-3-{[9-Bromo-2-(4-methoxyphenyl)[1,2,4]triazolo[1,5-c]quinazolin-5-yl]amino}azepan-2-one (30.0 mg, 62.3 μmol), lithium carbonate (27.6 mg, 374 mmol) and bis{3,5-difluoro-2-[5-(trifluoromethyl)pyridin-2-yl]phenyl}iridium(1+) hexafluorophosphate(1−)-4,4′-di-tert-butyl[2,2′-bipyridine] (1:1:1) (1.40 mg, 1.25 μmol) were suspended in trifluorotoluene (1.2 mL) in a cylindrical reaction vial. In a separate flask, the Ni-catalyst was prepared by dissolving nickel (II) chloride dimethoxyethane adduct (68 μg, 0.31 μmol) and 4,4′-di-tert-butyl-2,2′-bipyridine (84 μg, 0.31 μmol) in N,N-dimethylacetamide (0.6 mL) followed by stirring for 5 min. The catalyst solution was syringed to the reaction vessel and argon was bubbled through the solution for 20 min. 2-Bromopropane (26 μL, 280 μmol,) and tris(trimethylsilyl)silane (19 μl, 62 μmol,) were added followed by irradiation using two Kessil LED Aquarium lights (40 W each, 4 cm distance) and a water bath to keep the temperature below 35° C. After 4 hours, the reaction was quenched by addition of water and the organic solvent was evaporated. The formed precipitate was filtered off and dried at 60° C. Purification by preparative HPLC afforded the title compound (3R)-3-{[2-(4-methoxyphenyl)-9-(propan-2-yl)[1,2,4]triazolo[1,5-c]quinazolin-5-yl]amino}azepan-2-one (8.4 mg, 18.9 μmol, 30%).

LC-MS (method 2): Rₜ=1.52 min; MS (ESIpos): m/z=445 [M+H]⁺

¹H NMR (DMSO-d₆, 400 MHz): δ ppm=8.18-8.26 (m, 3H), 8.11 (d, 1H), 7.59-7.68 (m, 3H), 7.12-7.17 (m, 2H), 4.82 (br dd, 1H), 3.86 (s, 3H), 3.35-3.42 (m, 1H), 3.04-3.22 (m, 2H), 2.52-2.55 (m, 1H), 2.27-2.35 (m, 1H), 1.97-2.08 (m, 1H), 1.81-1.96 (m, 2H), 1.48-1.63 (m, 1H), 1.31 (d, 6H).

### Example 393

### (3R)-3-{[2-(4-Fluorophenyl)-7-(propan-2-yl)[1,2,4]triazolo[1,5-c]quinazolin-5-yl]amino}azepan-2-one

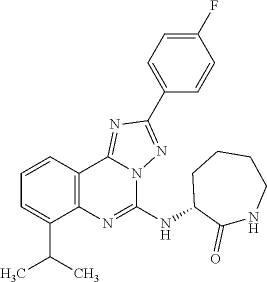 [SMILES: CC(C)c1cccc2c1nc(N[C@@H]1CCCCNC1=O)n1nc(-c3ccc(F)cc3)nc21]

In analogy to example 392, (3R)-3-{[7-bromo-2-(4-fluorophenyl)[1,2,4]triazolo[1,5-c]quinazolin-5-yl]amino}azepan-2-one (50.0 mg, 107 μmol), 2-bromopropane (45 μL, 480 μmol,), bis{3,5-difluoro-2-[5-(trifluoromethyl)pyridin-2-yl]phenyl}iridium(1+) hexafluorophosphate(1−)-4,4′-di-tert-butyl[2,2′-bipyridine] (1:1:1) (2.39 mg, 2.13 μmol]), lithium carbonate (47.2 mg, 639 μmol), nickel (II) chloride dimethoxyethane adduct (120 μg, 0.53 μmol), 4,4′-di-tert-butyl-2,2′-bipyridine (140 μg, 0.53 μmol) and tris(trimethylsilyl)silane (33 μl, 110 μmol) were reacted in a mixture of N,N-dimethylacetamide (1 mL) and trifluorotoluene (2 mL) to obtain after preparative HPLC purification the title compound (3R)-3-{[2-(4-fluorophenyl)-7-(propan-2-yl)[1,2,4]triazolo[1,5-c]quinazolin-5-yl]amino}azepan-2-one (22.3 mg, 51.5 μmol, 48%).

LC-MS (method 2): Rₜ=1.62 min; MS (ESIpos): m/z=433 [M+H]⁺

¹H NMR (DMSO-d₆, 400 MHz): δ ppm 8.24 (dd, 1H), 8.17 (dd, 1H), 8.14 (dt, 1H), 8.00 (dq, 1H), 7.61-7.74 (m, 3H), 7.39-7.49 (m, 2H), 4.83 (dd, 1H), 3.99 (spt, 1H), 3.35-3.40 (m, 1H), 3.14-3.24 (m, 1H), 2.41 (d, 1H), 2.00-2.11 (m, 1H), 1.81-1.95 (m, 2H), 1.57 (q, 1H), 1.39 (d, 3H), 1.37-1.31 (m, 4H).

### Example 394

### (3R)-3-{[2-(3-Fluorophenyl)-7-(propan-2-yl)[1,2,4]triazolo[1,5-c]quinazolin-5-yl]amino}azepan-2-one

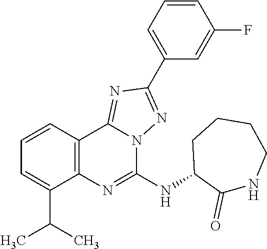 [SMILES: CC(C)c1cccc2c1nc(N[C@@H]1CCCCNC1=O)n1nc(-c3cccc(F)c3)nc21]

In analogy to example 392, (3R)-3-{[7-bromo-2-(3-fluorophenyl)[1,2,4]triazolo[1,5-c]quinazolin-5-yl]amino}azepan-2-one (50.0 mg, 107 μmol), 2-bromopropane (45 μl, 480 μmol,), bis{3,5-difluoro-2-[5-(trifluoromethyl)pyridin-2-yl]phenyl}iridium(1+) hexafluorophosphate(1−)-4,4′-di-tert-butyl[2,2′-bipyridine] (1:1:1) (2.39 mg, 2.13 μmol,), lithium carbonate (47.2 mg, 639 μmol), nickel (II) chloride dimethoxyethane adduct (120 μg, 0.53 μmol), 4,4′-di-tert-butyl-2,2′-bipyridine (140 μg, 0.53 μmol) and tris(trimethylsilyl)silane (33 μL, 110 μmol) were reacted in a mixture of N,N-dimethylacetamide (1 mL) and trifluorotoluene (2 mL) to obtain after preparative HPLC purification the title compound (3R)-3-{[2-(3-fluorophenyl)-7-(propan-2-yl)[1,2,4]triazolo[1,5-c]quinazolin-5-yl]amino}azepan-2-one (22.1 mg, 51.1 μmol, 48%).

LC-MS (method 2): Rₜ=1.60 min; MS (ESIpos): m/z=433 [M+H]⁺

¹H NMR (DMSO-d₆, 400 MHz): δ ppm 8.30-8.36 (m, 2H), 8.24 (dd, 1H), 8.16 (dd, 1H), 7.62-7.70 (m, 2H), 7.40-7.47 (m, 3H), 4.83 (dd, 1H), 3.99 (spt, 1H), 3.34-3.42 (m, 1H), 3.14-3.25 (m, 1H), 2.41 (d, 1H), 2.00-2.10 (m, 1H), 1.88 (d, 2H), 1.50-1.64 (m, 1H), 1.39 (d, 3H), 1.37-1.31 (m, 4H).

### Example 395

### (3R)-3-{[2-(4-Methoxyphenyl)-8-(propan-2-yl)[1,2,4]triazolo[1,5-c]quinazolin-5-yl]amino}azepan-2-one

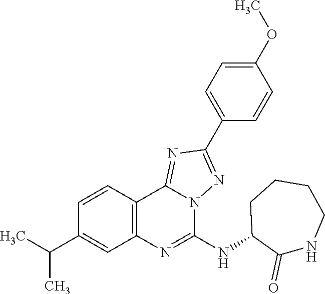 [SMILES: COc1ccc(-c2nc3c4ccc(C(C)C)cc4nc(N[C@@H]4CCCCNC4=O)n3n2)cc1]

In analogy to example 392, (3R)-3-{[8-bromo-2-(4-methoxyphenyl)[1,2,4]triazolo[1,5-c]quinazolin-5-yl]amino}azepan-2-one (75.0 mg, 156 μmol), 2-bromopropane (66 μL, 700 μmol), bis{3,5-difluoro-2-[5-(trifluoromethyl)pyridin-2-yl]phenyl}iridium(1+) hexafluorophosphate(1−)-4,4′-di-tert-butyl[2,2′-bipyridine] (1:1:1) (3.50 mg, 3.12 μmol), lithium carbonate (69.1 mg, 935 μmol), nickel (II) chloride dimethoxyethane adduct (170 μg, 0.78 μmol), 4,4′-di-tert-butyl-2,2′-bipyridine (210 μg, 0.78 μmol) and tris(trimethylsilyl)silane (48 μL, 160 μmol) were reacted in a mixture of N,N-dimethylacetamide (1.5 mL) and trifluorotoluene (3 mL) to obtain after preparative HPLC purification the title compound (3R)-3-{[2-(4-methoxyphenyl)-8-(propan-2-yl)[1,2,4]triazolo[1,5-c]quinazolin-5-yl]amino}azepan-2-one (10.7 mg, 24.1 μmol, 15%).

LC-MS (method 2): Rₜ=1.50 min; MS (ESIpos): m/z=445 [M+H]⁺

¹H NMR (DMSO-d₆, 400 MHz): δ ppm 8.17-8.26 (m, 4H), 7.67 (d, 1H), 7.50 (d, 1H), 7.37 (dd, 1H), 7.10-7.17 (m, 2H), 4.83 (dd, 1H), 3.86 (s, 3H), 3.34-3.42 (m, 1H), 3.13-3.23 (m, 1H), 3.02-3.13 (m, 1H), 2.28-2.35 (m, 1H), 2.26-2.31 (m, 1H), 1.82-1.95 (m, 2H), 1.51-1.64 (m, 1H), 1.26-1.39 (m, 7H).

### Example 396

### (3R)-3-{[2-(4-methoxyphenyl)-10-(oxetan-3-yl)[1,2,4]triazolo[1,5-c]quinazolin-5-yl]amino}azepan-2-one

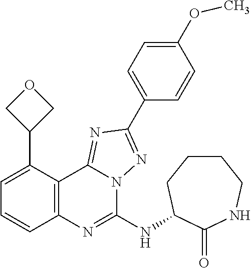 [SMILES: COc1ccc(-c2nc3c4c(C5COC5)cccc4nc(N[C@@H]4CCCCNC4=O)n3n2)cc1]

In analogy to example 392, (3R)-3-{[10-bromo-2-(4-methoxyphenyl)[1,2,4]triazolo[1,5-c]quinazolin-5-yl]amino}azepan-2-one (50.0 mg, 104 μmol), 3-bromooxetane (39 μl, 470 μmol), Ir(4′,6′-dF-5-CF₃-ppy)₂(4,4′-dtbbpy)PF₆ (2.33 mg, 2.08 μmol), lithium carbonate (46.1 mg, 623 μmol), Nickel (II) chloride dimethoxyethane adduct (110 μg, 0.52 μmol), 4,4′-di-tert-butyl-2,2′-bipyridine (140 μg, 0.52 μmol) and tris(trimethylsilyl)silane (32 μl, 100 μmol) were reacted in a mixture of N,N-dimethylacetamide (1.0 ml) and trifluorotoluene (2.0 ml) to obtain after preparative HPLC purification the title compound (3R)-3-{[2-(4-methoxyphenyl)-10-(oxetan-3-yl)[1,2,4]triazolo[1,5-c]quinazolin-5-yl]amino}azepan-2-one (21.0 mg, 45.8 μmol, 40%).

LC-MS (method 2): Rₜ=1.31 min; MS (ESIpos): m/z=459 [M+H]⁺

¹H NMR (DMSO-d6, 400 MHz): δ ppm 8.18-8.27 (m, 3H), 7.69-7.77 (m, 2H), 7.58 (d, 1H), 7.54 (d, 1H), 7.16 (d, 2H), 5.41 (quin, 1H), 5.25 (dd, 2H), 4.76-4.86 (m, 3H), 3.34-3.41 (m, 1H), 3.11-3.21 (m, 1H), 2.26-2.36 (m, 1H), 1.98-2.07 (m, 1H), 1.80-1.94 (m, 2H), 1.50-1.64 (m, 1H), 1.25-1.39 (m, 1H).

### Example 397

### (3R)-3-{[2-(4-methoxyphenyl)-10-(oxan-4-yl)[1,2,4]triazolo[1,5-c]quinazolin-5-yl]amino}azepan-2-one

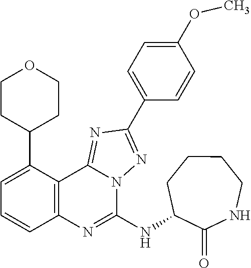 [SMILES: COc1ccc(-c2nc3c4c(C5CCOCC5)cccc4nc(N[C@@H]4CCCCNC4=O)n3n2)cc1]

In analogy to example 392, (3R)-3-{[10-bromo-2-(4-methoxyphenyl)[1,2,4]triazolo[1,5-c]quinazolin-5-yl]amino}azepan-2-one (50.0 mg, 104 μmol), 4-bromooxane (53 μl, 470 μmol), Ir(4′,6′-dF-5-CF₃-ppy)₂(4,4′-dtbbpy)PF₆ (2.33 mg, 2.08 μmol), lithium carbonate (46.1 mg, 623 μmol), Nickel (II) chloride dimethoxyethane adduct (110 μg, 0.52 μmol), 4,4′-di-tert-butyl-2,2′-bipyridine (140 μg, 0.52 μmol) and tris(trimethylsilyl)silane (32 μl, 100 μmol) were reacted in a mixture of N,N-dimethylacetamide (1.0 ml) and trifluorotoluene (2.0 ml) to obtain after preparative HPLC purification the title compound (3R)-3-{[2-(4-methoxyphenyl)-10-(oxan-4-yl)[1,2,4]triazolo[1,5-c]quinazolin-5-yl]amino}azepan-2-one (24.0 mg, 49.3 μmol, 45%).

LC-MS (method 2): Rₜ=1.43 min; MS (ESIpos): m/z=487 [M+H]⁺

¹H NMR (DMSO-d₆, 400 MHz): δ ppm 8.18-8.27 (m, 3H), 7.69-7.73 (m, 1H), 7.67 (d, 1H), 7.53 (dd, 1H), 7.37 (d, 1H), 7.18 (d, 2H), 4.82 (br dd, 1H), 4.72 (tt, 1H), 4.08 (br d, 2H), 3.70 (br t, 2H), 3.34-3.43 (m, 1H), 3.09-3.22 (m, 1H), 2.26-2.36 (m, 1H), 1.72-2.09 (m, 7H), 1.49-1.63 (m, 1H), 1.25-1.39 (m, 1H).

### Example 398

### (3R)-3-({2-(4-methoxyphenyl)-7-[1-(trifluoromethyl)cyclopropyl][1,2,4]triazolo[1,5-c]quinazolin-5-yl}amino)azepan-2-one

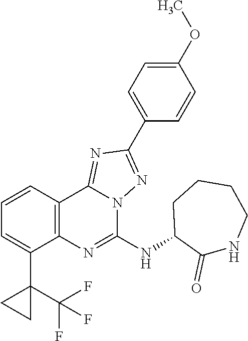 [SMILES: COc1ccc(-c2nc3c4cccc(C5(C(F)(F)F)CC5)c4nc(N[C@@H]4CCCCNC4=O)n3n2)cc1]

(3R)-3-{[2-(4-methoxyphenyl)-7-(3,3,3-trifluoroprop-1-en-2-yl)[1,2,4]triazolo[1,5-c]quinazolin-5-yl]amino}azepan-2-one (25.0 mg, 50.4 μmol, Intermediate 388), triethylammonium bis(catecholato)iodomethylsilicate (56.4 mg, 116 μmol) and 2,4,5,6-Tetra(9H-carbazol-9-yl)isophthalonitrile (1.19 mg, 1.51 μmol) were dissolved in DMSO (1 mL) followed by sparging with argon for 5 min. The reaction mixture was placed in a water bath and irradiated by two Kessil LED Aquarium lights (40 W each, 4 cm distance) for 12 h. The reaction was quenched by the addition of 1M NaOH and ethyl acetate. The layers were separated and the aqueous phase was extracted twice with ethyl acetate following filtering over a hydrophobic filter. After evaporation the crude material was purified by preparative HPLC to afford the title compound (3R)-3-({2-(4-methoxyphenyl)-7-[1-(trifluoromethyl)cyclopropyl][1,2,4]triazolo[1,5-c]quinazolin-5-yl}amino)azepan-2-one (5.0 mg, 9.1 μmol, 18% yield).

LC-MS (method 2): Rₜ=1.50 min; MS (ESIpos): m/z=511 [M+H]⁺

¹H NMR (DMSO-d6, 400 MHz): δ ppm 8.34 (dd, 1H), 8.25-8.30 (m, 1H), 8.23 (d, 2H), 7.87 (dd, 1H), 7.77 (d, 1H), 7.47 (t, 1H), 7.15 (d, 2H), 4.80 (dd, 1H), 3.86 (s, 3H), 3.16-3.30 (m, 2H), 1.95-2.09 (m, 1H), 1.78-1.92 (m, 2H), 1.46-1.65 (m, 3H), 1.25-1.38 (m, 3H).

### Example 399

### (3R)-3-{[2-(4-methoxyphenyl)-7-(1-methylcyclopropyl)[1,2,4]triazolo[1,5-c]quinazolin-5-yl]amino}azepan-2-one

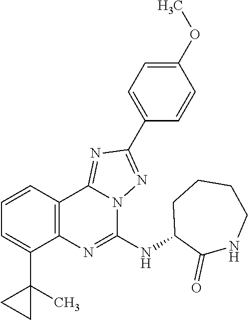 [SMILES: COc1ccc(-c2nc3c4cccc(C5(C)CC5)c4nc(N[C@@H]4CCCCNC4=O)n3n2)cc1]

In analogy to example 398, (3R)-3-{[2-(4-methoxyphenyl)-7-(prop-1-en-2-yl)[1,2,4]triazolo[1,5-c]quinazolin-5-yl]amino}azepan-2-one (25.0 mg, 56.5 μmol, Intermediate 389), triethylammonium bis(catecholato)iodomethylsilicate (63.3 mg, 130 μmol) and 2,4,5,6-Tetra(9H-carbazol-9-yl)isophthalonitrile (1.34 mg, 1.69 μmol) were reacted in DMSO (1 ml) to obtain after preparative HPLC purification the title compound (3R)-3-{[2-(4-methoxyphenyl)-7-(1-methylcyclopropyl)[1,2,4]triazolo[1,5-c]quinazolin-5-yl]amino}azepan-2-one (3.0 mg, 6.6 μmol, 12%).

LC-MS (method 2): Rₜ=1.56 min; MS (ESIpos): m/z=457 [M+H]⁺

¹H NMR (DMSO-d6, 400 MHz): δ ppm 8.20-8.28 (m, 3H), 8.17 (dd, 1H), 7.70 (d, 1H), 7.66 (dd, 1H), 7.35 (t, 1H), 7.14 (d, 2H), 4.85 (br dd, 1H), 3.86 (s, 3H), 3.15-3.32 (m, 2H), 2.00-2.14 (m, 1H), 1.82-2.00 (m, 2H), 1.56-1.64 (m, 1H), 1.53 (s, 3H), 1.25-1.42 (m, 1H), 0.70-0.89 (m, 4H).

### Example 400

### 2-(4-Methoxyphenyl)-5-{[(3R)-2-oxoazepan-3-yl]amino}[1,2,4]triazolo[1,5-c]quinazoline-8-carbonitrile

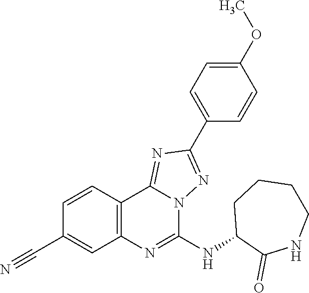 [SMILES: COc1ccc(-c2nc3c4ccc(C#N)cc4nc(N[C@@H]4CCCCNC4=O)n3n2)cc1]

(3R)-3-{[8-Bromo-2-(4-methoxyphenyl)[1,2,4]triazolo[1,5-c]quinazolin-5-yl]amino}azepan-2-one (53 mg, 0.11 mmol), bis[cinnamyl palladium(II) chloride] (2.9 mg, 0.006 mmol), 1,1′-ferrocenediyl-bis(diphenylphosphine) (3.0 mg, 0.006 mmol) and zinc cyanide (15.7 mg, 0.13 mmol) were added to a 5 ml reaction vessel and the vessel sealed and flushed with argon. Degassed N,N-dimethylacetamid (1 ml) and N,N-diisopropylethylamin (39 μl, 0.27 mmol), were added and the mixture heated overnight at 80° C. The mixture was cooled to RT, the precipitate filtered and washed with water. The mixture was diluted with DCM, washed with NaHCO₃ (saturated aqueous solution), and the aqueous phase extracted twice with DCM. The combined organic phases were dried passed through a water repellent filter, and purified by RP-HPLC (column: X-Bridge C18 5μ 100×30 mm; acetonitrile/water+0.1% ammonia), yielding the title compound (25.9 mg, 0.06 mmol, 58%).

LC-MS (method 2): Rₜ=1.29 min; MS (ESIneg): m/z=426.3 [M−H]⁻.

¹H-NMR (400 MHz, DMSO-d6) δ ppm 8.43, 8.41, 8.24, 8.22, 8.11, 7.94, 7.92, 7.79, 7.76, 7.76, 7.17, 7.15, 4.85, 4.84, 4.84, 4.83, 4.82, 3.90, 3.88, 3.86, 3.68, 3.68, 3.66, 3.66, 3.64, 3.64, 3.63, 3.62, 3.60, 3.60, 3.58, 3.57, 3.57, 3.53, 3.50, 3.46, 3.25, 3.23, 3.23, 3.22, 3.21, 3.21, 3.20, 3.18, 3.18, 3.17, 3.15, 3.15, 3.14, 3.14, 3.11, 3.10, 3.10, 3.09, 3.08, 3.08, 3.07, 3.07, 3.06, 3.06, 3.06, 3.04, 3.03, 3.02, 3.02, 3.01, 3.01, 3.00, 2.99, 2.99, 2.98, 2.98, 2.97, 2.96, 2.96, 2.94, 2.93, 2.93, 2.92, 2.91, 2.91, 2.89, 2.87, 2.86, 2.85, 2.85, 2.84, 2.83, 2.83, 2.82, 2.81, 2.81, 2.80, 2.79, 2.79, 2.78, 2.77, 2.77, 2.77, 2.72, 2.71, 2.67, 2.65, 2.42, 2.41, 2.39, 2.37, 2.36, 2.33, 2.29, 2.26, 2.25, 2.24, 2.24, 2.23, 2.21, 2.20, 2.20, 2.19, 2.18, 2.17, 2.17, 2.15, 2.15, 2.15, 2.14, 2.13, 2.13, 2.12, 2.11, 2.11, 2.10, 2.09, 2.09, 2.08, 2.07, 2.05, 2.05, 2.03, 2.03, 2.02, 2.01, 1.99, 1.98, 1.97, 1.96, 1.95, 1.94, 1.94, 1.93, 1.92, 1.90, 1.89, 1.88, 1.86, 1.85, 1.81, 1.81, 1.81, 1.81, 1.80, 1.79, 1.79, 1.78, 1.77, 1.63, 1.63, 1.62, 1.61, 1.60, 1.57, 1.56, 1.55, 1.54, 1.53, 1.52, 1.47, 1.46, 1.46, 1.45, 1.45, 1.38, 1.37, 1.34, 1.31, 1.30, 1.30, 1.30, 1.29, 1.28, 1.28, 1.27, 1.26, 1.26, 1.23, 1.21, 0.85, 0.06, 0.05

### Example 401

### 2-(4-Methoxyphenyl)-5-{[(3R)-2-oxoazepan-3-yl]amino}[1,2,4]triazolo[1,5-c]quinazoline-9-carbonitrile

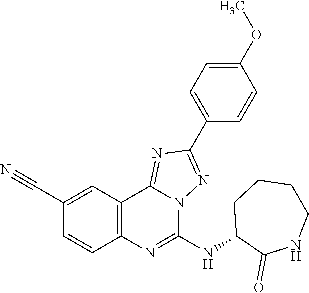 [SMILES: COc1ccc(-c2nc3c4cc(C#N)ccc4nc(N[C@@H]4CCCCNC4=O)n3n2)cc1]

(3R)-3-{[9-bromo-2-(4-methoxyphenyl)[1,2,4]triazolo[1,5-c]quinazolin-5-yl]amino}azepan-2-one (50 mg, 0.10 mmol), bis[cinnamyl palladium(II) chloride] (2.7 mg, 0.005 mmol), 1,1′-ferrocenediyl-bis(diphenylphosphine) (2.9 mg, 0.006 mmol) and zinc cyanide (12.2 mg, 0.1 mmol) were added to a 5 ml reaction vessel and the vessel sealed and flushed with argon. Degassed N,N-dimethylacetamid (1 ml) and N,N-diisopropylethylamin (36 μl, 0.27 mmol), were added and the mixture heated overnight at 80° C. The mixture was cooled to RT, the precipitate filtered and washed with water. The mixture was diluted with DCM, washed with NaHCO₃ (saturated aqueous solution), and the aqueous phase extracted twice with DCM. The combined organic phases were dried passed through a water repellent filter, and purified by RP-HPLC (column: X-Bridge C18 5μ 100×30 mm; acetonitrile/water+0.1% ammonia), yielding the title compound (12.3 mg, 0.03 mmol, 27%).

LC-MS (method 2): Rₜ=1.27 min; MS (ESIpos): m/z=428.18 [M+H]⁺

¹H-NMR (400 MHz, ACETONITRILE-d3) δ ppm 1.13-1.50 (m, 2H) 2.11-2.27 (m, 1H) 3.01-3.23 (m, 2H) 3.66 (s, 3H) 4.65 (dd, 1H) 6.42-6.53 (m, 1H) 6.88 (d, 2H) 7.47-7.73 (m, 1H) 7.56-7.64 (m, 1H) 7.64-7.74 (m, 1H) 7.96-8.13 (m, 2H) 8.36-8.55 (m, 1H)

### Example 402

### 2-(4-Fluorophenyl)-5-{[(3R)-2-oxoazepan-3-yl]amino}[1,2,4]triazolo[1,5-c]quinazoline-7-carbonitrile

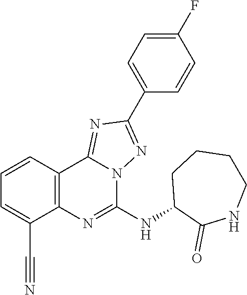 [SMILES: N#Cc1cccc2c1nc(N[C@@H]1CCCCNC1=O)n1nc(-c3ccc(F)cc3)nc21]

(3R)-3-{[9-bromo-2-(4-methoxyphenyl)[1,2,4]triazolo[1,5-c]quinazolin-5-yl]amino}azepan-2-one (50 mg, 0.10 mmol), bis[cinnamyl palladium(II) chloride] (2.7 mg, 0.005 mmol), 1,1′-ferrocenediyl-bis(diphenylphosphine) (2.9 mg, 0.006 mmol) and zinc cyanide (12.2 mg, 0.1 mmol) were added to a 5 ml reaction vessel and the vessel sealed and flushed with argon. Degassed N,N-dimethylacetamid (1 ml) and N,N-diisopropylethylamin (36 μl, 0.27 mmol), were added and the mixture heated overnight at 80° C. The mixture was cooled to RT, added to water and the precipitate filtered and washed with water. Purified was performed by RP-H PLC (column: X-Bridge C18 5μ 100×30 mm; acetonitrile/water+0.1% formic acid), yielding the title compound (15.5 mg, 0.04 mmol, 34%).

LC-MS (method 1): Rₜ=1.33 min; MS (ESIpos): m/z=416.2 [M+H]⁺

¹H-NMR (400 MHz, DMSO-d₆): δ ppm 1.28-1.40 (m, 1H) 1.52-1.65 (m, 1H) 1.83-2.11 (m, 3H) 2.37-2.45 (m, 1H) 3.14-3.31 (m, 2H) 4.82-4.90 (m, 1H) 7.41-7.50 (m, 1H) 7.56 (t, 1H) 7.67 (td, 1H) 7.96-8.02 (m, 1H) 8.03-8.09 (m, 1H) 8.11-8.17 (m, 1H) 8.22-8.31 (m, 2H) 8.55-8.60 (m, 1H)

### Example 403

### 2-(1-Methyl-1H-pyrazol-4-yl)-5-{[(3R)-2-oxoazepan-3-yl]amino}[1,2,4]triazolo[1,5-c]quinazoline-7-carbonitrile

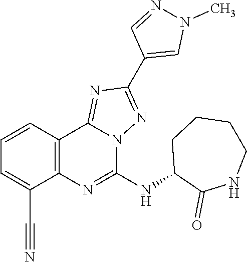 [SMILES: Cn1cc(-c2nc3c4cccc(C#N)c4nc(N[C@@H]4CCCCNC4=O)n3n2)cn1]

10-Bromo-2-(4-fluorophenyl)[1,2,4]triazolo[1,5-c]quinazolin-5(6H)-one (50 mg, 0.11 mmol), bis[cinnamyl palladium(II) chloride] (2.8 mg, 0.005 mmol), 1,1′-ferrocenediyl-bis(diphenylphosphine) (3 mg, 0.005 mmol) and zinc cyanide (12.9 mg, 0.11 mmol) were added to a 5 ml reaction vessel and the vessel sealed and flushed with argon. Degassed N,N-dimethylacetamid (1 mL) and N,N-diisopropylethylamin (38 μL, 0.22 mmol), were added and the mixture heated overnight at 80° C. The mixture was cooled to RT, the mixture was diluted with water and the precipitate filtered, dried and purified by RP-HPLC (column: X-Bridge C18 5μ 100×30 mm; acetonitrile/water+0.1% formic acid), yielding the title compound (5.9 mg, 0.01 mmol, 11%).

LC-MS (method 1): Rₜ=1.01 min; MS (ESIpos): m/z=402.4 [M+H]⁺

¹H-NMR (400 MHz, ACETONITRILE-d3) δ ppm 1.13-1.47 (m, 2H) 1.77-1.90 (m, 2H) 2.21-2.31 (m, 1H) 3.02-3.20 (m, 2H) 3.70-3.79 (m, 3H) 4.61-4.69 (m, 1H) 6.43-6.55 (m, 1H) 7.23-7.29 (m, 1H) 7.50-7.57 (m, 1H) 7.83-7.88 (m, 2H) 7.97-8.01 (m, 1H) 8.24-8.30 (m, 1H).

### Example 404

### (3R)-3-{[10-(difluoromethyl)-9-methyl-2-(1-methyl-1H-pyrazol-4-yl)[1,2,4]triazolo[1,5-c]quinazolin-5-yl]amino}azepan-2-one

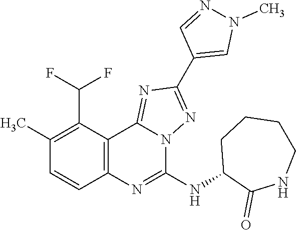 [SMILES: Cc1ccc2nc(N[C@@H]3CCCCNC3=O)n3nc(-c4cnn(C)c4)nc3c2c1C(F)F]

(3R)-3-{[9-methyl-2-(1-methyl-1H-pyrazol-4-yl)[1,2,4]triazolo[1,5-c]quinazolin-5-yl]amino}azepan-2-one (200 mg, 0.51 mmol), zinc difluoromethanesulfinate (1.2 g, 4.1 mmol), were added to a 5 ml reaction vessel, the vessel was sealed and the flask flushed with argon. Acetonitrile (2 ml), was added, followed by tert-Butyl hydroperoxide (70% in water, 701 μl, 5.2 mmol) was added dropwise and the mixture was stirred overnight. The mixture was filtered through a 2 g silica column, the column washed with DCM/MeOH (9:1), and the filtrate concentrated under reduced pressure. The residue was purified by RP-HPLC (column: X-Bridge C18 5μ 100×30 mm; acetonitrile/water+0.1% formic acid), followed by a second purification using a Waters Autopurification+SQD; Column: YMC Cellulose SC 5μ 250×30; Eluent A: Water+0.1% Formic acid; Eluent B: Acetonitril; Gradient: 30-90% B in 20 min; Flow: 40 ml/min; Temperature: 25° C.; UV: DAD 210-400 nm, yielding the title compound (1.0 mg, 1%).

LC-MS (method 1): Rₜ=1.31 min; MS (ESIpos): m/z=441.4 [M+H]⁺

¹H-NMR (500 MHz, DMSO-d6) δ ppm 9.30, 9.19, 9.08, 8.58, 8.33, 8.25, 8.24, 8.24, 8.23, 8.12, 8.12, 7.76, 7.74, 7.69, 7.69, 7.68, 4.84, 4.83, 4.82, 4.81, 4.59, 4.59, 4.10, 4.03, 3.98, 3.95, 3.53, 3.52, 3.51, 3.50, 3.48, 3.47, 3.47, 3.46, 3.44, 3.43, 3.42, 3.39, 3.37, 3.31, 3.19, 3.19, 3.18, 3.18, 3.16, 2.67, 2.66, 2.66, 2.65, 2.65, 2.65, 2.54, 2.54, 2.53, 2.48, 2.47, 2.44, 2.43, 2.43, 2.42, 2.40, 2.39, 2.39, 2.38, 2.38, 2.38, 2.37, 2.34, 2.33, 2.30, 2.30, 2.19, 2.08, 2.06, 2.05, 2.04, 2.03, 2.02, 2.01, 2.01, 2.00, 1.99, 1.92, 1.91, 1.90, 1.90, 1.88, 1.86, 1.57, 1.55, 1.53, 1.52, 1.51, 1.51, 1.35, 1.35, 1.31, 1.30, 1.29, 1.29, 1.29, 1.29, 1.28, 1.25, 1.21, 1.20, 1.20, 1.19, 1.12, 1.07, 0.88, 0.87, 0.86, 0.10, 0.09, 0.08, 0.07, 0.06, 0.02

### Example 405

### (3R)-3-{[8-(difluoromethyl)-9-methyl-2-(1-methyl-1H-pyrazol-4-yl)[1,2,4]triazolo[1,5-c]quinazolin-5-yl]amino}azepan-2-one

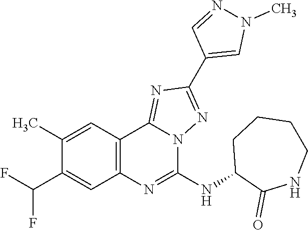 [SMILES: Cc1cc2c(cc1C(F)F)nc(N[C@@H]1CCCCNC1=O)n1nc(-c3cnn(C)c3)nc21]

(3R)-3-{[9-methyl-2-(1-methyl-1H-pyrazol-4-yl)[1,2,4]triazolo[1,5-c]quinazolin-5-yl]amino}azepan-2-one (200 mg, 0.51 mmol), zinc difluoromethanesulfinate (1.2 g, 4.1 mmol), were added to a 5 ml reaction vessel, the vessel was sealed and the flask flushed with argon. Acetonitrile (2 ml), was added, followed by tert-Butyl hydroperoxide (70% in water, 701 μl, 5.2 mmol) was added dropwise and the mixture was stirred overnight. The mixture was filtered through a 2 g silica column, the column washed with DCM/MeOH (9:1), and the filtrate concentrated under reduced pressure. The residue was purified by RP-HPLC (column: X-Bridge C18 5μ 100×30 mm; acetonitrile/water+0.1% formic acid), followed by a second purification using a Waters Autopurification+SQD; Column: YMC Cellulose SC 5μ 250×30; Eluent A: Water+0.1% Formic acid; Eluent B: Acetonitril; Gradient: 30-90% B in 20 min; Flow: 40 ml/min; Temperature: 25° C.; UV: DAD 210-400 nm, yielding the title compound (4.1 mg, 1.8%).

LC-MS (method 1): Rₜ=1.17 min; MS (ESIpos): m/z=441.4 [M+H]⁺

¹H-NMR (400 MHz, DMSO-d₆): δ [ppm]=8.51, 8.49, 8.31, 8.23, 8.22, 8.22, 8.21, 8.16, 8.07, 8.07, 7.80, 7.65, 7.64, 7.57, 7.40, 7.29, 7.18, 5.75, 4.84, 4.83, 4.82, 4.81, 4.80, 4.58, 4.57, 4.26, 4.25, 3.97, 3.96, 3.53, 3.53, 3.53, 3.51, 3.51, 3.49, 3.48, 3.47, 3.46, 3.44, 3.44, 3.42, 3.41, 3.40, 3.38, 3.38, 3.36, 3.35, 3.34, 3.30, 3.18, 3.17, 3.15, 3.15, 3.14, 2.65, 2.56, 2.54, 2.52, 2.52, 2.52, 2.47, 2.47, 2.37, 2.37, 2.32, 2.32, 2.29, 2.04, 2.02, 2.01, 2.00, 2.00, 2.00, 1.99, 1.99, 1.90, 1.89, 1.87, 1.85, 1.85, 1.84, 1.84, 1.56, 1.53, 1.51, 1.49, 1.49, 1.47, 1.45, 1.44, 1.34, 1.32, 1.31, 1.30, 1.30, 1.29, 1.28, 1.28, 1.27, 1.26, 1.24, 1.21, 1.21, 1.19, 1.11, 1.07, 1.06, 1.04, 0.87, 0.85, 0.84, 0.12, 0.08, 0.07, 0.07, 0.06, 0.05, 0.02, 0.02, 0.01

### Example 406

### (3R)-3-{[7-(difluoromethyl)-9-methyl-2-(1-methyl-1H-pyrazol-4-yl)[1,2,4]triazolo[1,5-c]quinazolin-5-yl]amino}azepan-2-one

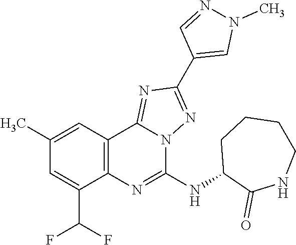 [SMILES: Cc1cc(C(F)F)c2nc(N[C@@H]3CCCCNC3=O)n3nc(-c4cnn(C)c4)nc3c2c1]

(3R)-3-{[9-methyl-2-(1-methyl-1H-pyrazol-4-yl)[1,2,4]triazolo[1,5-c]quinazolin-5-yl]amino}azepan-2-one (200 mg, 0.51 mmol), zinc difluoromethanesulfinate (1.2 g, 4.1 mmol), were added to a 5 ml reaction vessel, the vessel was sealed and the flask flushed with argon. Acetonitrile (2 ml), was added, followed by tert-Butyl hydroperoxide (70% in water, 701 μl, 5.2 mmol) was added dropwise and the mixture was stirred overnight. The mixture was filtered through a 2 g silica column, the column washed with DCM/MeOH (9:1), and the filtrate concentrated under reduced pressure. The residue was purified by RP-HPLC (column: X-Bridge C18 5μ 100×30 mm; acetonitrile/water+0.1% formic acid), followed by a second purification using a Waters Autopurification+SQD; Column: YMC Cellulose SC 5μ 250×30; Eluent A: Water+0.1% Formic acid; Eluent B: Acetonitril; Gradient: 30-90% B in 20 min; Flow: 40 ml/min; Temperature: 25° C.; UV: DAD 210-400 nm, yielding the title compound (3.8 mg, 1.5%).

LC-MS (method 1): Rₜ=1.24 min; MS (ESIpos): m/z=441.4 [M+H]⁺

¹H-NMR (600 MHz, DMSO-d6) δ 8.49, 8.32, 8.25, 8.24, 8.24, 8.23, 8.21, 8.07, 7.82, 7.80, 7.71, 7.65, 7.64, 7.61, 4.87, 4.86, 4.85, 4.84, 3.96, 3.42, 3.41, 3.40, 3.39, 3.39, 3.38, 3.37, 3.32, 3.18, 3.18, 3.17, 3.15, 3.15, 2.62, 2.56, 2.54, 2.53, 2.52, 2.52, 2.46, 2.45, 2.39, 2.33, 2.31, 2.31, 2.03, 2.02, 2.00, 2.00, 1.96, 1.96, 1.94, 1.93, 1.92, 1.91, 1.89, 1.87, 1.87, 1.86, 1.85, 1.84, 1.55, 1.53, 1.52, 1.51, 1.50, 1.49, 1.36, 1.35, 1.35, 1.35, 1.34, 1.33, 1.31, 1.31, 1.30, 1.30, 1.29, 1.29, 1.28, 1.28, 1.27, 1.26, 1.25, 1.23, 1.19, 1.11, 1.07, 1.06, 1.04, 1.00, 0.97, 0.95, 0.95, 0.94, 0.87, 0.86, 0.85, 0.84, 0.83, 0.82, 0.82, 0.81, 0.80, 0.10, 0.01

### Example 407

### (3R)-3-({9-methyl-2-[1-methyl-3-(trifluoromethyl)-1H-pyrazol-4-yl][1,2,4]triazolo[1,5-c]quinazolin-5-yl}amino)azepan-2-one

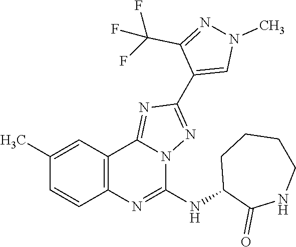 [SMILES: Cc1ccc2nc(N[C@@H]3CCCCNC3=O)n3nc(-c4cn(C)nc4C(F)(F)F)nc3c2c1]

(3R)-3-{[9-Methyl-2-(1-methyl-1H-pyrazol-4-yl)[1,2,4]triazolo[1,5-c]quinazolin-5-yl]amino}azepan-2-one (200 mg, 0.51 mmol), sodium trifluoromethanesulfinate (639 mg, 4.1 mmol), copper(II) trifluoromethanesulfonate (37 mg, 0.1 mmol) were added to a 5 ml reaction vessel, the vessel was sealed and the flask flushed with argon. Acetonitrile (2 ml), was added, followed by tert-Butyl hydroperoxide (70% in water, 561 μl, 4.1 mmol) which was added dropwise and the mixture was stirred overnight. The mixture was filtered through a 2 g silica column, and the filtrate concentrated under reduced pressure. The residue was purified by RP-HPLC (column: X-Bridge C18 5μ 100×30 mm; acetonitrile/water+0.1% formic acid), followed by a second purification using a Waters Autopurification+SQD system; Column: YMC Cellulose SC 5μ 250×30 mm; Eluent A: Water+0.1% Formic acid; Eluent B: Acetonitril; Gradient: 40->80% B in 20 min; Flow: 40 ml/min; Temperature: 25° C.; DAD scan: 210-400 nm, yielding the title compound (2.9 mg, 1.2%).

LC-MS (method 1): Rₜ=1.30 min; MS (ESIpos): m/z=459.4 [M+H]⁺

1H NMR (600 MHz, DMSO-d6) δ ppm 8.30, 8.20, 8.19, 8.18, 8.05, 8.05, 7.61, 7.60, 7.59, 7.58, 7.58, 7.56, 7.56, 5.74, 4.81, 4.80, 4.79, 4.78, 4.14, 4.14, 3.96, 3.37, 3.36, 3.34, 3.30, 3.18, 3.16, 3.16, 3.15, 2.63, 2.62, 2.62, 2.62, 2.58, 2.56, 2.56, 2.54, 2.53, 2.52, 2.52, 2.46, 2.46, 2.46, 2.39, 2.39, 2.34, 2.32, 2.04, 2.04, 2.01, 1.90, 1.87, 1.85, 1.56, 1.54, 1.52, 1.50, 1.35, 1.33, 1.31, 1.30, 1.29, 1.28, 1.28, 1.26, 1.24, 1.22, 0.87, 0.86, 0.84, 0.10, 0.01, −0.01, −0.10

### Example 408

### (6R)-6-({2-[3-(dimethylamino)phenyl][1,2,4]triazolo[1,5-c]quinazolin-5-yl}amino)-1,4-diazepan-5-one

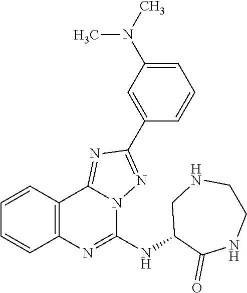 [SMILES: CN(C)c1cccc(-c2nc3c4ccccc4nc(N[C@@H]4CNCCNC4=O)n3n2)c1]

Benzyl (6R)-6-({2-[3-(dimethylamino)phenyl][1,2,4]triazolo[1,5-c]quinazolin-5-yl}amino)-5-oxo-1,4-diazepane-1-carboxylate (108 mg, 196 μmol) was dissolved in DCM (2.0 mL) and treated with hydrogen bromide (350 μL, 33% purity in AcOH, 2.0 mmol). The mixture was stirred 2 h at rt and purified by preparative HPLC to give 33.6 mg (94% purity, 39% yield) of the target compound.

LC-MS (Method 2): Rₜ=1.08 min; MS (ESIpos): m/z=417 [M+H]⁺

¹H-NMR (400 MHz, DMSO-d₆): δ [ppm]=2.70 (dd, 1H), 2.93-3.06 (m, 8H), 3.07-3.16 (m, 1H), 3.37-3.48 (m, 2H), 4.89 (ddd, 1H), 6.89-6.95 (m, 1H), 7.38 (t, 1H), 7.46 (ddd, 1H), 7.56-7.62 (m, 2H), 7.66-7.72 (m, 2H), 7.72-7.78 (m, 1H), 8.28 (dd, 1H), 8.32 (dd, 1H).

The following examples were prepared similarly to example 408:

| | Structure |
|---|---|
| | IUPAC-Name |
| | LC-MS (method): Retention time; Mass found |
| Example | ¹H-NMR |
| Example 409 | 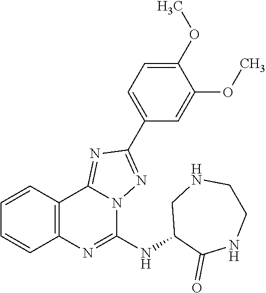 [SMILES: COc1ccc(-c2nc3c4ccccc4nc(N[C@@H]4CNCCNC4=O)n3n2)cc1OC] |
| | (6R)-6-{[2-(3,4-dimethoxyphenyl)[1,2,4]triazolo[1,5-c]quinazolin-5- |
| | yl]amino}-1,4-diazepan-5-one |
| | LC-MS (Method 2): Rₜ = 0.95 min; MS (ESIpos): m/z = 434 [M + H]⁺ |
| | ¹H-NMR (400 MHz, DMSO-d₆): δ [ppm] = 2.73 (dd, 1H), 3.05 (br dd, 1H), 3.09- |
| | 3.17 (m, 1H), 3.39-3.49 (m, 2H), 3.86 (s, 3H), 3.91 (s, 3H), 4.91 (td, 1H), |
| | 7.17 (d, 1H), 7.46 (ddd, 1H), 7.66-7.71 (m, 2H), 7.72-7.77 (m, 1H), 7.78 (d, |
| | 1H), 7.88 (dd, 1H), 8.26-8.33 (m, 2H). |
| Example 410 | 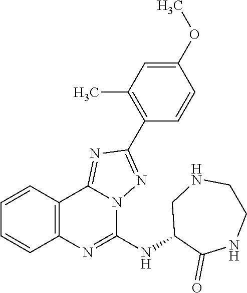 [SMILES: COc1ccc(-c2nc3c4ccccc4nc(N[C@@H]4CNCCNC4=O)n3n2)c(C)c1] |
| | (6R)-6-{[2-(4-methoxy-2-methylphenyl)[1,2,4]triazolo[1,5-c]quinazolin-5- |
| | yl]amino}-1,4-diazepan-5-one |
| | LC-MS (Method 2): Rₜ = 1.09 min; MS (ESIpos): m/z = 418 [M + H]⁺ |
| | ¹H-NMR (400 MHz, DMSO-d₆): δ [ppm] = 2.63-2.71 (m, 1H), 2.74 (s, 3H), 3.02 |
| | (br dd, 1H), 3.07-3.16 (m, 1H), 3.38-3.47 (m, 2H), 3.84 (s, 3H), 4.87 (ddd, |
| | 1H), 6.95-7.00 (m, 2H), 7.46 (ddd, 1H), 7.66-7.71 (m, 2H), 7.72-7.77 (m, |
| | 1H), 8.11-8.16 (m, 1H), 8.25-8.32 (m, 2H). |
| Example 411 | 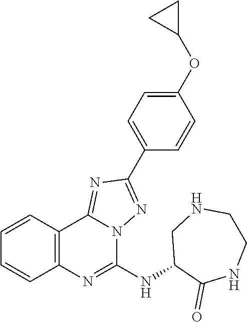 [SMILES: O=C1NCCNC[C@H]1Nc1nc2ccccc2c2nc(-c3ccc(OC4CC4)cc3)nn12] |
| | (6R)-6-({2-[4-(cyclopropyloxy)phenyl][1,2,4]triazolo[1,5-c]quinazolin-5- |
| | yl}amino)-1,4-diazepan-5-one |
| | LC-MS (Method 2): Rₜ = 1.14 min; MS (ESIpos): m/z = 430 [M + H]⁺ |
| | ¹H-NMR (400 MHz, DMSO-d₆): δ [ppm] = 0.69-0.75 (m, 2H), 0.81-0.88 (m, |
| | 2H), 2.64-2.72 (m, 1H), 3.03 (br dd, 1H), 3.07-3.16 (m, 1H), 3.38-3.48 (m, |
| | 2H), 3.95 (tt, 1H), 4.88 (ddd, 1H), 7.23-7.28 (m, 2H), 7.45 (td, 1H), 7.65-7.70 |
| | (m, 2H), 7.71-7.77 (m, 1H), 8.20-8.25 (m, 2H), 8.27-8.33 (m, 2H). |
| Example 412 | 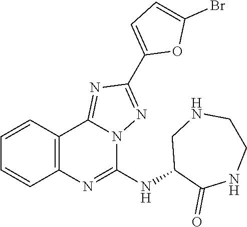 [SMILES: O=C1NCCNC[C@H]1Nc1nc2ccccc2c2nc(-c3ccc(Br)o3)nn12] |
| | (6R)-6-{[2-(5-bromofuran-2-yl)[1,2,4]triazolo[1,5-c]quinazolin-5- |
| | yl]amino}-1,4-diazepan-5-one |
| | LC-MS (Method 2): Rₜ = 1.03 min; MS (ESIpos): m/z = 442 [M + H]⁺ |
| | ¹H-NMR (400 MHz, DMSO-d₆): δ [ppm] = 2.52-2.55 (m, 1H), 2.63-2.71 (m, |
| | 1H), 2.97-3.15 (m, 2H), 3.35-3.46 (m, 2H), 4.87 (ddd, 1H), 6.90 (d, 1H), 7.36 |
| | (d, 1H), 7.46 (ddd, 1H), 7.64-7.71 (m, 2H), 7.75 (dd, 1H), 8.24-8.33 (m, 2H). |
| Example 413 | 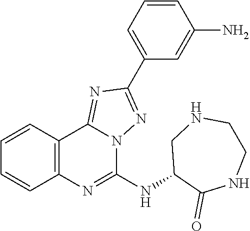 [SMILES: Nc1cccc(-c2nc3c4ccccc4nc(N[C@@H]4CNCCNC4=O)n3n2)c1] |
| | (6R)-6-{[2-(3-aminophenyl)[1,2,4]triazolo[1,5-c]quinazolin-5-yl]amino}- |
| | 1,4-diazepan-5-one |
| | LC-MS (Method 2): Rt = 0.75 min; MS (ESIpos): m/z = 389 [M + H]⁺ |
| | ¹H-NMR (400 MHz, DMSO-d₆): δ [ppm] = 2.61-2.71 (m, 1H), 3.02 (br dd, 1H), |
| | 3.07-3.17 (m, 1H), 3.37-3.48 (m, 2H), 4.88 (ddd, 1H), 5.41 (s, 2H), 6.72 |
| | (ddd, 1H), 7.21 (t, 1H), 7.41-7.49 (m, 2H), 7.55 (t, 1H), 7.63 (d, 1H), 7.67- |
| | 7.71 (m, 1H), 7.72-7.78 (m, 1H), 8.28 (dd, 1H), 8.32 (dd, 1H). |
| Example 414 | 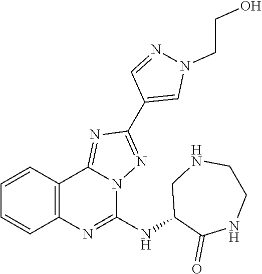 [SMILES: O=C1NCCNC[C@H]1Nc1nc2ccccc2c2nc(-c3cnn(CCO)c3)nn12] |
| | (6R)-5-({2-[1-(2-hydroxyethyl)-1H-pyrazol-4-yl][1,2,4]triazolo[1,5- |
| | c]quinazolin-5-yl}amino)-1,4-diazepan-5-one |
| | LC-MS (Method 2): Rₜ = 0.69 min; MS (ESIpos): m/z = 408 [M + H]⁺ |
| | ¹H-NMR (400 MHz, DMSO-d₆): δ [ppm] = 2.62-2.69 (m, 2H), 2.98-3.05 (m, |
| | 1H), 3.06-3.17 (m, 1H), 3.37-3.47 (m, 2H), 3.81 (q, 2H), 4.26 (t, 2H), 4.87 |
| | (br dd, 1H), 4.99 (t, 1H), 7.44 (ddd, 1H), 7.57 (d, 1H), 7.65-7.70 (m, 1H), 7.71- |
| | 7.77 (m, 1H), 8.09 (d, 1H), 8.24-8.27 (m, 1H), 8.27-8.32 (m, 1H), 8.47 (s, |
| | 1H). |
| Example 415 | 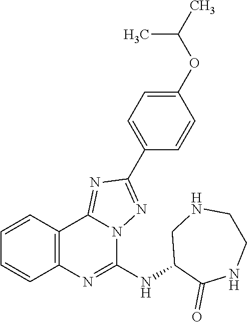 [SMILES: CC(C)Oc1ccc(-c2nc3c4ccccc4nc(N[C@@H]4CNCCNC4=O)n3n2)cc1] |
| | (6R)-6-[(2-{4-[(propan-2-yl)oxy]phenyl}[1,2,4]triazolo[1,5-c]quinazolin-5- |
| | yl)amino]-1,4-diazepan-5-one |
| | LC-MS (Method 2): Rₜ = 1.19 min; MS (ESIpos): m/z = 432 [M + H]⁺ |
| | ¹H-NMR (400 MHz, DMSO-d₆): δ [ppm] = 1.32 (d, 6H), 2.67 (dd, 1H), 2.95 (br |
| | s, 1H), 3.02 (br dd, 1H), 3.07-3.16 (m, 1H), 3.37-3.47 (m, 2H), 2.74 (spt, |
| | 1H), 4.87 (ddd, 1H), 7.08-7.15 (m, 2H), 7.45 (ddd, 1H), 7.65-7.70 (m, 2H), |
| | 7.70-7.80 (m, 1H), 8.16-8.24 (m, 2H), 8.26-8.34 (m, 2H). |
| Example 416 | 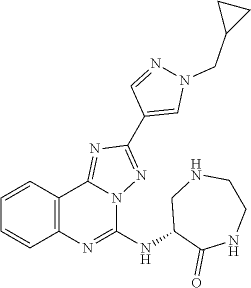 [SMILES: O=C1NCCNC[C@H]1Nc1nc2ccccc2c2nc(-c3cnn(CC4CC4)c3)nn12] |
| | (6R)-6-({2-[1-(cyclopropylmethyl)-1H-pyrazol-4-yl][1,2,4]triazolo[1,5- |
| | c]quinazolin-5-yl}amino)-1,4-diazepan-5-one |
| | LC-MS (Method 2): Rₜ = 0.90 min; MS (ESIpos): m/z = 418 [M + H]⁺ |
| | ¹H-NMR (400 MHz, DMSO-d₆): δ [ppm] = 0.41-0.46 (m, 2H), 0.54-0.60 (m, |
| | 2H), 1.29-1.39 (m, 1H), 2.62-2.69 (m, 1H), 3.02 (br dd, 1H), 3.07-3.15 (m, |
| | 1H), 3.36-3.47 (m, 2H), 4.08 (d, 2H), 4.87 (ddd, 1H), 7.45 (ddd, 1H), 7.57 (d, |
| | 1H), 7.66-7.70 (m, 1H), 7.73 (ddd, 1H), 8.09 (d, 1H), 8.26 (dd, 1H), 8.30 (dd, |
| | 1H), 8.54 (s, 1H). |
| Example 417 | 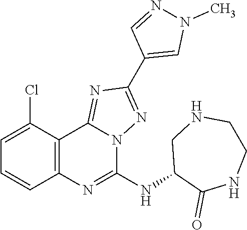 [SMILES: Cn1cc(-c2nc3c4c(Cl)cccc4nc(N[C@@H]4CNCCNC4=O)n3n2)cn1] |
| | (6R)-6-{[10-chloro-2-(1-methyl-1H-pyrazol-4-yl[1,2,4]triazolo[1,5- |
| | c]quinazolin-5-yl]amino}-1,4-diazepan-5-one |
| | LC-MS (Method 2): Rₜ = 0.86 min; MS (ESIpos): m/z = 412 [M + H]⁺ |
| | ¹H-NMR (400 MHz, DMSO-d₆): δ [ppm] = 2.61-2.64 (m, 3H), 2.64-2.68 |
| | (m, 1H), 2.78-2.97 (m, 1H), 2.97-3.16 (m, 2H), 3.36-3.52 (m, 2H), |
| | 3.95 (s, 3H), 2.86 (br dd, 1H), 7.33 (t, 1H), 7.52 (d, 1H), 7.58-7.65 (m, |
| | 1H), 8.06 (d, 1H), 8.08 (dd, 1H), 8.29 (dd, 1H), 8.48 (s, 1H). |
| Example 418 | 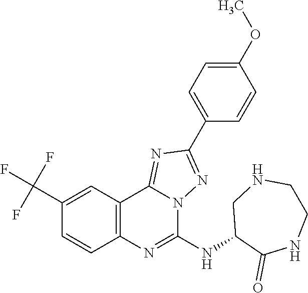 [SMILES: COc1ccc(-c2nc3c4cc(C(F)(F)F)ccc4nc(N[C@@H]4CNCCNC4=O)n3n2)cc1] |
| | (6R)-6-{[2-(4-methoxyphenyl)-9-(trifluoromethyl)[1,2,4]triazolo[1,5- |
| | c]quinazolin-5-yl]amino}-1,4-diazepan-5-one |
| | LC-MS (Method 2): Rₜ = 1.24 min; MS (ESIpos): m/z = 472 [M + H]⁺ |
| | ¹H-NMR (400 MHz, DMSO-d₆): δ [ppm] = 2.66-2.75 (m, 1H), 2.95 (br s, 1H), |
| | 3.03 (br dd, 1H), 3.07-3.16 (m, 1H), 3.38-3.48 (m, 2H), 3.86 (s, 3H), 4.92 |
| | (br d, 1H), 7.13-7.18 (m, 2H), 7.83 (d, 1H), 7.89-7.97 (m, 1H), 8.01 (dd, 1H), |
| | 8.21-8.26 (m, 2H), 8.32 (dd, 1H), 8.52 (d, 1H). |
| Example 419 | 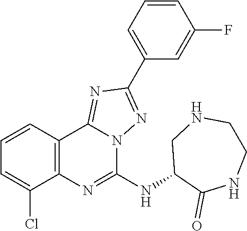 [SMILES: O=C1NCCNC[C@H]1Nc1nc2c(Cl)cccc2c2nc(-c3cccc(F)c3)nn12] |
| | (6R)-6-{[7-chloro-2-(3-fluorophenyl)[1,2,4]triazolo[1,5-c]quinazolin-5- |
| | yl]amino}-1,4-diazepan-5-one |
| | LC-MS (Method 2): Rₜ = 1.21 min; MS (ESIpos): m/z = 426 [M + H]⁺ |
| | ¹H-NMR (400 MHz, DMSO-d₆): δ [ppm] = 2.69-2.83 (m, 2H), 3.04 (br d, 1H), |
| | 3.08-3.17 (m, 1H), 3.37-3.47 (m, 1H), 3.51 (br d, 1H), 4.88-4.95 (m, 1H), |
| | 7.41-7.47 (m, 2H), 7.67 (td, 1H), 7.92 (br d, 2H), 7.97-8.03 (m, 1H), 8.14 (dt, |
| | 1H), 8.26-8.33 (m, 2H). |
| Example 420 | 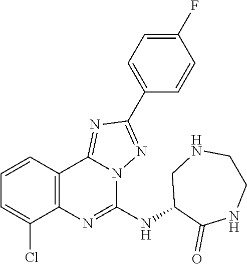 [SMILES: O=C1NCCNC[C@H]1Nc1nc2c(Cl)cccc2c2nc(-c3ccc(F)cc3)nn12] |
| | (6R)-6-{[7-chloro-2-(4-fluorophenyl)[1,2,4]triazolo[1,5-c]quinazolin-5- |
| | yl]amino]-1,4-diazepan-5-one |
| | LC-MS (Method 2): Rₜ = 1.21 min; MS (ESIpos): m/z = 426 [M + H]⁺ |
| | ¹H-NMR (400 MHz, DMSO-d₆): δ [ppm] = 2.68-2.81 (m, 2H), 3.04 (br d, 1H), |
| | 3.08-3.17 (m, 1H), 3.37-3.46 (m, 1H), 3.52 (br d, 1H), 4.87-4.94 (m, 1H), |
| | 7.39-7.48 (m, 3H), 7.88-7.94 (m, 2H), 8.24-8.36 (m, 4H). |
| Example 421 | 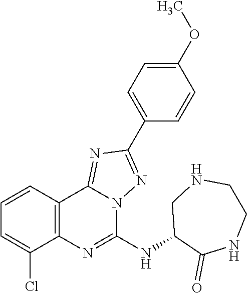 [SMILES: COc1ccc(-c2nc3c4cccc(Cl)c4nc(N[C@@H]4CNCCNC4=O)n3n2)cc1] |
| | (6R)-6-{[7-chloro-2-(4-methoxyphenyl)[1,2,4]triazolo[1,5-c]quinazolin-5- |
| | yl]amino)-1,4-diazepan-5-one |
| | LC-MS (Method 2): Rₜ = 1.14 min; MS (ESIpos): m/z = 438 [M + H]⁺ |
| | ¹H-NMR (400 MHz, DMSO-d₆): δ [ppm] = 2.72 (br d, 1H), 3.04 (br dd, 1H), 3.07- |
| | 3.17 (m, 1H), 3.37-3.46 (m, 1H), 3.51 (br d, 1H), 3.86 (s, 3H), 4.87-4.94 |
| | (m, 1H), 7.15 (d, 2H), 7.42 (t, 1H), 7.86 (d, 1H), 7.90 (dd, 1H), 8.20-8.32 (m, |
| | 4H). |
| Example 422 | 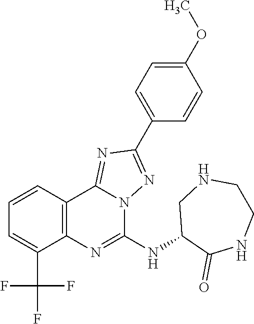 [SMILES: COc1ccc(-c2nc3c4cccc(C(F)(F)F)c4nc(N[C@@H]4CNCCNC4=O)n3n2)cc1] |
| | (6R)-6-{[2-(4-methoxyphenyl)-7-(trifluoromethyl)[1,2,4]triazolo[1,5- |
| | c]quinazolin-5-yl]amino}-1,4-diazepan-5-one |
| | LC-MS (Method 2): Rₜ = 1.23 min; MS (ESIpos): m/z = 472 [M + H]⁺ |
| | ¹H-NMR (400 MHz, DMSO-d₆): δ [ppm] = 2.63 (br s, 1H), 2.72 (dd, 1H), 3.05 |
| | (br dd, 1H), 3.09-3.18 (m, 1H), 3.31-3.41 (m, 1H and water signal), 3.49 (dd, |
| | 1H), 3.86 (s, 3H), 4.84 (br dd, 1H), 7.12-7.18 (m, 2H), 7.55 (t, 1H), 7.95 (d, |
| | 1H), 8.08-8.12 (m, 1H), 8.20-8.25 (m, 2H), 8.29 (dd, 1H), 8.55 (d, 1H). |
| Example 423 | 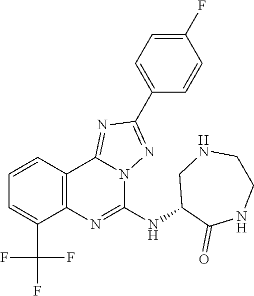 [SMILES: O=C1NCCNC[C@H]1Nc1nc2c(C(F)(F)F)cccc2c2nc(-c3ccc(F)cc3)nn12] |
| | (6R)-6-{[2-(4-fluorophenyl)-7-(trifluoromethyl)[1,2,4]triazolo[1,5- |
| | c]quinazolin-5-yl]amino}-1,4-diazepan-5-one |
| | LC-MS (Method 2): Rₜ = 1.25 min; MS (ESIpos): m/z = 460 [M + H]⁺ |
| | ¹H-NMR (400 MHz, DMSO-d₆): δ [ppm] = 2.73 (br dd, 1H), 3.05 (br dd, 1H), |
| | 3.09-3.18 (m, 1H), 3.35-3.43 (m, 1H), 3.49 (dd, 1H), 4.81-4.88 (m, 1H), |
| | 7.40-7.48 (m, 2H), 7.57 (t, 1H), 8.00 (d, 1H), 8.11 (dd, 1H), 8.26-8.31 (m, |
| | 1H), 8.32-8.37 (m, 2H), 8.56 (dd, 1H). |
| Example 424 | 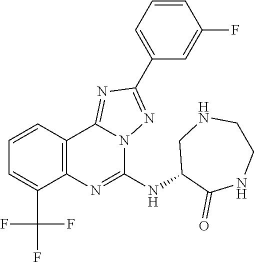 [SMILES: O=C1NCCNC[C@H]1Nc1nc2c(C(F)(F)F)cccc2c2nc(-c3cccc(F)c3)nn12] |
| | (6R)-6-{[2-(3-fluorophenyl)-7-(trifluoromethyl)[1,2,4]triazolo[1,5- |
| | c]quinazolin-5-yl]amino}-1,4-diazepan-5-one |
| | LC-MS (Method 2): Rₜ = 1.26 min; MS (ESIneg): m/z = 458 [M − H]⁻ |
| | ¹H-NMR (400 MHz, DMSO-d₆): δ [ppm] = 2.74 (dd, 1H), 3.05 (dd, 1H), 3.13 |
| | (ddd, 1H), 3.35-3.42 (m, 1H), 3.48 (dd, 1H), 4.85 (td, 1H), 7.40-7.48 (m, 1H), |
| | 7.57 (t, 1H), 7.67 (td, 1H), 7.97-8.06 (m, 2H), 8.09-8.16 (m, 2H), 8.29 (dd, |
| | 1H), 8.57 (dd, 1H). |
| Example 425 | 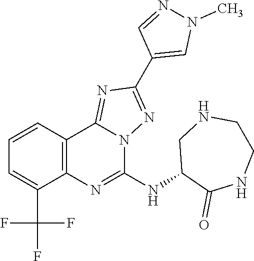 [SMILES: Cn1cc(-c2nc3c4cccc(C(F)(F)F)c4nc(N[C@@H]4CNCCNC4=O)n3n2)cn1] |
| | (6R)-6-{[2-(1-methyl-1H-pyrazol-4-yl)-7- |
| | (trifluoromethyl)[1,2,4]triazolo[1,5-c]quinazolin-5-yl]amino}-1,4- |
| | diazepan-5-one |
| | LC-MS (Method 2): Rₜ = 0.93 min; MS (ESIpos): m/z = 446 [M + H]⁺ |
| | ¹H-NMR (400 MHz, DMSO-d₆): δ [ppm] = 2.63-2.73 (m, 2H), 3.01-3.16 9m, |
| | 2H), 3.36-3.51 (m, 2H), 3.96 (s, 3H), 4.82 (br dd, 1H), 7.54 (t, 1H), 7.84 (d, |
| | 1H), 8.07-8.11 (m, 2H), 8.29 (dd, 1H), 8.48-8.52 (m, 2H). |
| Example 426 | 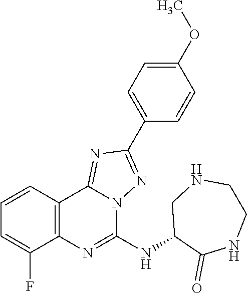 [SMILES: COc1ccc(-c2nc3c4cccc(F)c4nc(N[C@@H]4CNCCNC4=O)n3n2)cc1] |
| | (6R)-6-{[7-fluoro-2-(4-methoxyphenyl)[1,2,4]triazolo[1,5-c]quinazolin-5- |
| | yl]amino}-1,4-diazepan-5-one |
| | LC-MS (Method 2): Rₜ = 1.08 min; MS (ESIpos): m/z = 422 [M + H]⁺ |
| | ¹H-NMR (400 MHz, DMSO-d₆): δ [ppm] = 2.64-2.74 (m, 1H), 2.82-2.90 (m, |
| | 1H), 2.98-3.06 (m, 1H), 3.07-3.16 (m, 1H), 3.37-3.47 (m, 2H), 3.86 (s, 3H), |
| | 4.84-4.92 (m, 1H), 7.12-7.18 (m, 2H), 7.42 (td, 1H), 7.61 (ddd, 1H), 7.85 (d, |
| | 1H), 8.11 (d, 1H), 8.19-8.25 (m, 2H), 8.30 (br dd, 1H). |
| Example 427 | 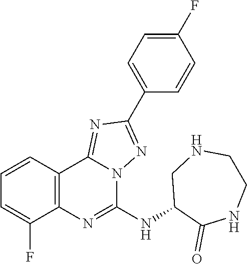 [SMILES: O=C1NCCNC[C@H]1Nc1nc2c(F)cccc2c2nc(-c3ccc(F)cc3)nn12] |
| | (6R)-6-{[7-fluoro-2-(4-fluorophenyl)[1,2,4]triazolo[1,5-c]quinazolin-5- |
| | yl]amino}-1,4-diazepan-5-one |
| | LC-MS (Method 2): Rₜ = 1.12 min; MS (ESIpos): m/z = 410 [M + H]⁺ |
| | ¹H-NMR (400 MHz, DMSO-d₆): δ [ppm] = 2.69 (dd, 1H), 3.02 (dd, 1H), 3.07- |
| | 3.16 (m, 1H), 3.41-3.47 (m, 2H), 4.89 (ddd, 1H), 7.39-7.49 (m, 3H), 7.62 |
| | (ddd, 1H), 7.88 (d, 1H), 8.09-8.14 (m, 1H), 8.27-8.36 (m, 3H). |
| Example 428 | 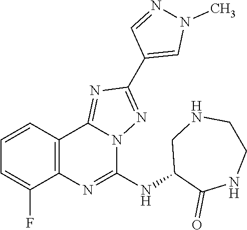 [SMILES: Cn1cc(-c2nc3c4cccc(F)c4nc(N[C@@H]4CNCCNC4=O)n3n2)cn1] |
| | (6R)-6-{[7-fluoro-2-(1-methyl-1H-pyrazol-4-yl[1,2,4]triazolo[1,5- |
| | c]quinazolin-5-yl]amino}-1,4-diazepan-5-one |
| | LC-MS (Method 2): Rt = 0.77 min; MS (ESIpos): m/z = 396 [M + H]⁺ |
| | ¹H-NMR (400 MHz, DMSO-d₆): δ [ppm] = 2.61-2.72 (m, 2H), 3.02 (br dd, 1H), |
| | 3.06-3.15 (m, 1H), 3.36-3.47 (m, 2H), 3.95 (s, 3H), 4.87 (ddd, 1H), 7.41 (td, |
| | 1H), 7.60 (ddd, 1H), 7.73 (d, 1H), 8.03-8.08 (m, 2H), 8.30 (dd, 1H), 8.50 (s, |
| | 1H). |
| Example 429 | 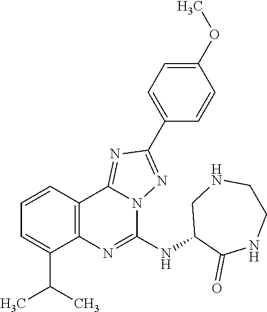 [SMILES: COc1ccc(-c2nc3c4cccc(C(C)C)c4nc(N[C@@H]4CNCCNC4=O)n3n2)cc1] |
| | (6R)-6-{[2-(4-methoxyphenyl)-7-(propan-2-yl)[1,2,4]triazolo[1,5- |
| | c]quinazolin-5-yl]amino}-1,4-diazepan-5-one |
| | LC-MS (Method 2): Rₜ = 1.28 min; MS (ESIpos): m/z = 446 [M + H]⁺ |
| | ¹H-NMR (400 MHz, DMSO-d₆): δ [ppm] = 1.32 (d, 3H), 1.37 (d, 3H), 2.53-2.60 |
| | (m, 1H), 2.75 (br dd, 1H), 3.04-3.19 (m, 2H), 3.45 (ddd, 1H), 3.52 (br d, 1H), |
| | 3.86 (s, 3H), 4.03 (spt, 1H), 4.90 (br dd, 1H), 7.12-7.17 (m, 2H), 7.42 (t, 1H), |
| | 7.65 (dd, 1H), 7.68 (d, 1H), 8.15 (dd, 1H), 8.20-8.25 (m, 2H), 8.30 (dd, 1H). |
| Example 430 | 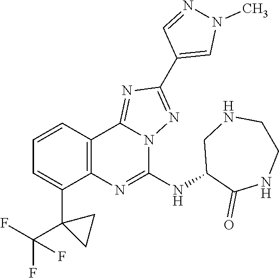 [SMILES: Cn1cc(-c2nc3c4cccc(C5(C(F)(F)F)CC5)c4nc(N[C@@H]4CNCCNC4=O)n3n2)cn1] |
| | (6R)-6-({2-(1-methyl-1H-pyrazol-4-yl)-7-[1- |
| | (trifluoromethyl)cyclopropyl[[1,2,4]triazolo[1,5-c]quinazolin-5-yl}amino)- |
| | 1,4-diazepan-5-one |
| | LC-MS (Method 2): Rₜ = 1.01 min; MS (ESIpos): m/z = 486 [M + H]⁺ |
| Example 431 | 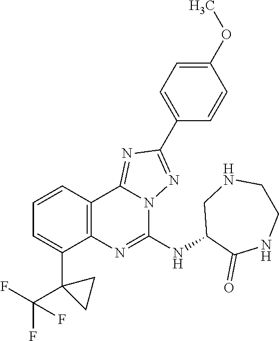 [SMILES: COc1ccc(-c2nc3c4cccc(C5(C(F)(F)F)CC5)c4nc(N[C@@H]4CNCCNC4=O)n3n2)cc1] |
| | (6R)-6-({2-(4-methoxyphenyl)-7-[1- |
| | (trifluoromethyl)cyclopropyl][1,2,4]triazolo[1,5-c]quinazolin-5-yl}amino)- |
| | 1,4-diazepan-5-one |
| | LC-MS (Method 2): Rₜ = 1.29 min; MS (ESIpos): m/z = 512 [M + H]⁺ |
| | ¹H-NMR (400 MHz, DMSO-d₆): δ [ppm] = 1.56-1.62 (m, 1H), 1.63-1.70 (m, |
| | 1H), 2.41-2.46 (m, 1H), 2.71 (br dd, 1H), 3.06 (br dd, 1H), 3.16 (br s, 1H), |
| | 3.36-3.41 (m, 1H), 3.51 (dd, 1H), 3.86 (s, 3H), 4.85 (br d, 1H), 7.13-7.17 |
| | (m, 2H), 7.47 (t, 1H), 7.75 (s, 1H), 7.86 (dd, 1H), 8.19-8.26 (m, 2H), 8.28- |
| | 8.31 (m, 1H), 8.34 (dd, 1H). |

### Example 432

### (3R)-3-({2-[1-(2-methoxyethyl)-1H-pyrazol-4-yl][1,2,4]triazolo[1,5-c]quinazolin-5-yl}amino)azepan-2-one

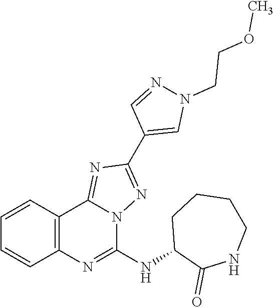 [SMILES: COCCn1cc(-c2nc3c4ccccc4nc(N[C@@H]4CCCCNC4=O)n3n2)cn1]

5-Chloro-2-[1-(2-methoxyethyl)-1H-pyrazol-4-yl][1,2,4]triazolo[1,5-c]quinazoline (75.0 mg, 228 μmol), (3R)-3-aminoazepan-2-one (32.2 mg, 251 μmol) and DIPEA (120 μL, 680 μmol) were stirred in DMSO for 2 h at 60° C. Water was added to the mixture, filtered, washed with water and dried under reduced pressure at 60° C. to give 34.9 mg (98% purity, 36% yield) of the target compound.

LC-MS (Method 2): Rₜ=1.05 m; MS (ESIpos): m/z=421 [M+H]⁺

¹H-NMR (400 MHz, DMSO-d₆): δ [ppm]=1.26-1.38 (m, 1H), 1.48-1.60 (m, 1H), 1.81-1.94 (m, 2H), 1.98-2.06 (m, 1H), 2.27-2.35 (m, 1H), 3.11-3.20 (m, 1H), 3.27 (s, 3H), 3.33-3.40 (m, 1H and water signal), 3.76 (t, 2H), 4.39 (t, 2H), 4.83 (br dd, 1H), 7.44 (ddd, 1H), 7.62 (d, 1H), 7.64-7.67 (m, 1H), 7.73 (ddd, 1H), 8.10 (d, 1H), 8.21 (dd, 1H), 8.25 (dd, 1H), 8.48 (d, 1H).

The following examples were prepared similarly to example 432:

| | Structure |
|---|---|
| | IUPAC-Name |
| | LC-MS (method): Retention time; Mass found |
| Example | ¹H-NMR |
| Example 433 | 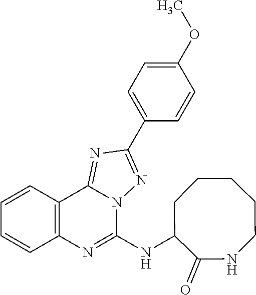 [SMILES: COc1ccc(-c2nc3c4ccccc4nc(NC4CCCCCNC4=O)n3n2)cc1] |
| | 3-{[2-(4-methoxyphenyl)[1,2,4]triazolo[1,5-c]quinazolin-5- |
| | yl]amino}azocan-2-one |
| | LC-MS (Method 2): Rt = 1.32 min; MS (ESIpos): m/z = 417 [M + H]⁺ |
| | ¹H-NMR (400 MHz, DMSO-d₆): δ [ppm] = 1.48-1.84 (m, 7H), 2.21-2.30 (m, |
| | 1H), 3.17-3.26 (m, 1H), 3.60-3.72 (m, 1H), 3.86 (s, 3H), 5.09-5.16 (m, |
| | 1H), 7.12-7.17 (m, 2H), 7.44 (ddd, 1H), 7.60-7.66 (m, 2H), 7.73 (ddd, 1H), |
| | 7.87 (dd, 1H), 8.22-8.26 (m, 2H), 8.29 (dd, 1H). |
| Example 434 | 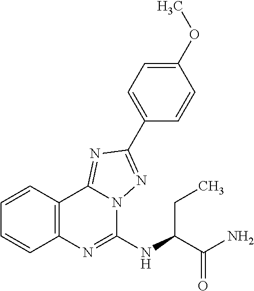 [SMILES: CC[C@H](Nc1nc2ccccc2c2nc(-c3ccc(OC)cc3)nn12)C(N)=O] |
| | (2S)-2-{[2-(4-methoxyphenyl)[1,2,4]triazolo[1,5-c]quinazolin-5- |
| | yl]amino}butanamide |
| | LC-MS (Method 2): Rt = 1.15 min; MS (ESIpos): m/z = 377 [M + H]⁺ |
| | ¹H-NMR (400 MHz, DMSO-d6): δ [ppm] = 0.96 (t, 3H), 1.87-2.00 (m, 1H), |
| | 2.01-2.13 (m, 1H), 3.86 (s, 3H), 4.70 (td, 1H), 7.12-7.17 (m, 2H), 7.32 (s, |
| | 1H), 7.44 (ddd, 1H), 7.54 (d, 1H), 7.62 (d, 1H), 7.70-7.75 (m, 2H), 8.23- |
| | 8.27 (m, 2H), 8.29 (dd, 1H). |
| Example 435 | 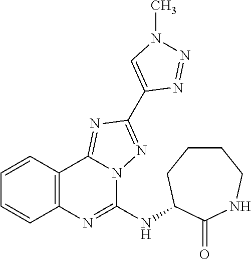 [SMILES: Cn1cc(-c2nc3c4ccccc4nc(N[C@@H]4CCCCNC4=O)n3n2)nn1] |
| | (3R)-3-{[2-(1-methyl-1H-1,2,3-triazol-4-yl[1,2,4]triazolo[1,5-c]quinazolin- |
| | 5-yl]amino}azepan-2-one |
| | LC-MS (Method 2): Rt = 0.97 min; MS (ESIpos): m/z = 378 [M + H]⁺ |
| | ¹H-NMR (400 MHz, DMSO-d₆): δ [ppm] = 1.26-1.39 (m, 1H), 1.49-1.61 (m, |
| | 1H), 1.81-1.95 (m, 2H), 1.98-2.07 (m, 1H), 2.30-2.38 (m, 1H), 3.12-3.21 |
| | (m, 1H), 4.18 (s, 3H), 4.83 (br dd, 1H), 7.44-7.49 (m, 1H), 7.66-7.70 (m, |
| | 1H), 7.71-7.78 (m, 2H), 8.22-8.29 (m, 2H), 8.86 (s, 1H). |
| Exampel 436 | 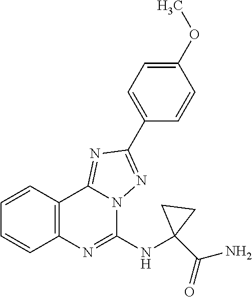 [SMILES: COc1ccc(-c2nc3c4ccccc4nc(NC4(C(N)=O)CC4)n3n2)cc1] |
| | 1-{[2-(4-methoxyphenyl)[1,2,4]triazolo[1,5-c]quinazolin-5- |
| | yl]amino}cyclopropane-1-carboxamide |
| | LC-MS (Method 2): Rₜ = 1.08 min; MS (ESIpos): m/z = 375 [M + H]⁺ |
| | ¹H-NMR (400 MHz, DMSO-d₆): δ [ppm] = 1.19-1.25 (m, 2H), 1.44-1.50 (m, |
| | 2H), 3.86 (s, 3H), 7.01 (s, 1H), 7.11-7.17 (m, 2H), 7.43 (ddd, 1H), 7.55 (s, |
| | 1H), 7.63-7.73 (m, 2H), 8.21-8.31 (m, 3H), 8.55 (s, 1H). |
| Example 437 | 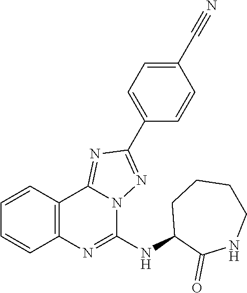 [SMILES: N#Cc1ccc(-c2nc3c4ccccc4nc(N[C@H]4CCCCNC4=O)n3n2)cc1] |
| | 4-(5-{[(3S)-2-oxoazepan-3-yl]amino}[1,2,4]triazolo[1,5-c]quinazolin-2- |
| | yl)benzonitrile |
| | LC-MS (Method 1): Rt = 1.43 min; MS (ESIpos): m/z = 398 [M + H]⁺ |
| | ¹H-NMR (400 MHz, DMSO-d₆): δ [ppm] = 1.26-1.39 (m, 1H), 1.51-1.63 (m, |
| | 1H), 1.82-1.95 (m, 2H), 1.98-2.08 (m, 1H), 2.29-2.36 (m, 1H), 3.12-3.21 |
| | (m, 1H), 3.32-3.41 (m, 1H and water signal), 4.84 (br dd, 1H), 7.47 (ddd, |
| | 1H), 7.67-7.70 (m, 1H), 7.73-7.81 (m, 2H), 8.05-8.09 (m, 2H), 8.23 (dd, |
| | 1H), 8.31 (dd, 1H), 8.43-8.47 (m, 2H). |
| Example 438 | 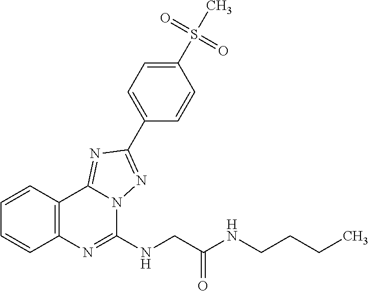 [SMILES: CCCCNC(=O)CNc1nc2ccccc2c2nc(-c3ccc(S(C)(=O)=O)cc3)nn12] |
| | N-butyl-N²-{2-[4-(methanesulfonyl)phenyl][1,2,4]triazolo[1,5- |
| | c]quinazolin-5-yl}glycinamide |
| | LC-MS (Method 2): RT = 1.09 min; MS (ESIpos): m/z = 453 [M + H]⁺ |
| | ¹H-NMR (400 MHz, DMSO-d₆): δ [ppm] = 0.84 (t, 3H), 1.22-1.32 (m, 2H), |
| | 1.34-1.43 (m, 2H), 3.09 (q, 2H), 3.32 (s, 3H), 4.15 (br d, 2H), 7.46 (ddd, |
| | 1H), 7.62 (d, 1H), 7.74 (ddd, 1H), 8.04 (t, 1H), 8.16-8.20 (m, 2H), 8.32 (dd, |
| | 1H), 8.36 (br t, 1H), 8.53-8.56 (m, 2H). |
| Example 439 | 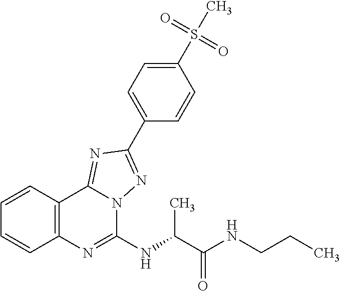 [SMILES: CCCNC(=O)[C@@H](C)Nc1nc2ccccc2c2nc(-c3ccc(S(C)(=O)=O)cc3)nn12] |
| | N²-{2-[4-(methanesulfonyl)phenyl][1,2,4]triazolo[1,5-c]quinazolin-5-yl}- |
| | N-propyl-D-alaninamide |
| | LC-MS (Method 2): Rt = 1.08 min; MS (ESIpos): m/z = 453 [M + H]⁺ |
| | ¹H-NMR (400 MHz, DMSO-d₆): δ [ppm] = 0.84 (t, 3H), 1.43 (sxt, 2H), 1.54 (d, |
| | 3H), 3.01-3.14 (m, 2H), 3.32 (s, 3H), 4.76 (quin, 1H), 7.47 (ddd, 1H), 7.63 |
| | (d, 1H), 7.75 (ddd, 1H), 7.91 (d, 1H), 8.15-8.20 (m, 3H), 8.32 (dd, 1H), 8.54- |
| | 8.58 (m, 2H). |
| Example 440 | 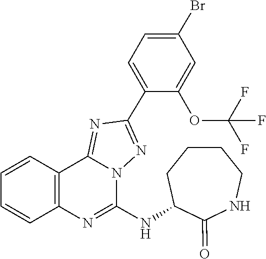 [SMILES: O=C1NCCCC[C@H]1Nc1nc2ccccc2c2nc(-c3ccc(Br)cc3OC(F)(F)F)nn12] |
| | (3R)-3-({2-[4-bromo-2-(trifluoromethoxy)phenyl][1,2,4]triazolo[1,5- |
| | c]quinazolin-5-yl}amino)azepan-2-one |
| | LC-MS (Method 2): Rt = 1.59 min; MS (ESIpos): m/z = 535 [M + H]⁺ |
| | ¹H-NMR (400 MHz, DMSO-d₆): δ [ppm] = 1.25-1.38 (m, 1H), 1.47-1.58 (m, |
| | 1H), 1.81-1.95 (m, 2H), 1.98-2.07 (m, 1H), 2.31-2.39 (m, 1H), 3.12-3.21 |
| | (m, 1H), 4.81 (br dd, 1H), 7.48 (ddd, 1H), 7.68-7.71 (m, 1H), 7.74-7.79 (m, |
| | 2H), 7.88-7.92 (m, 2H), 8.19 (dd, 1H), 8.28 (dd, 1H), 8.33-8.37 (m, 1H). |
| Example 441 | 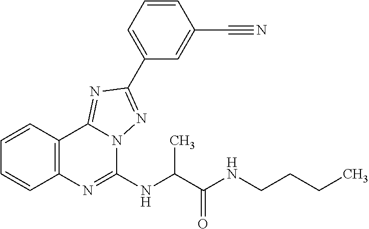 [SMILES: CCCCNC(=O)C(C)Nc1nc2ccccc2c2nc(-c3cccc(C#N)c3)nn12] |
| | N-butyl-N²-[2-(3-cyanophenyl)[1,2,4]triazolo[1,5-c]quinazolin-5- |
| | yl]alaninamide |
| | LC-MS (Method 2): Rt = 1.27 min; MS (ESIpos): m/z = 414 [M + H]⁺ |
| | ¹H-NMR (400 MHz, DMSO-d₆): δ [ppm] = 0.84 (t, 3H), 1.22-1.33 (m, 2H), |
| | 1.34-1.44 (m, 2H), 1.53 (d, 3H), 3.04-3.18 (m, 2H), 4.75 (quin, 1H), 7.46 |
| | (ddd, 1H), 7.62 (d, 1H), 7.75 (ddd, 1H), 7.83 (t, 1H), 7.90 (d, 1H), 8.05 (dt, |
| | 1H), 8.14 (t, 1H), 8.31 (dd, 1H), 8.60 (dt, 1H), 8.66 (t, 1H). |
| Example 442 | 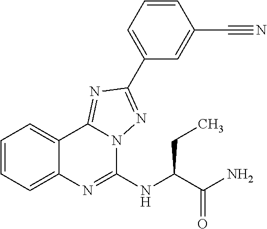 [SMILES: CC[C@H](Nc1nc2ccccc2c2nc(-c3cccc(C#N)c3)nn12)C(N)=O] |
| | (2S)-2-{[2-(3-cyanophenyl)[1,2,4]triazolo[1,5-c]quinazolin-5- |
| | yl]amino}butanamide |
| | LC-MS (Method 1): Rt = 1.13 min; MS (ESIpos): m/z = 372 [M + H]⁺ |
| | ¹H-NMR (400 MHz, DMSO-d₆): δ [ppm] = 0.98 (t, 3H), 1.88-2.00 (m, 1H), |
| | 2.02-2.13 (m, 1H), 4.70 (td, 1H), 7.32 (s, 1H), 7.43-7.49 (m, 1H), 7.63 (d, |
| | 1H), 7.69-7.77 (m, 3H), 7.82 (t, 1H), 8.05 (dt, 1H), 8.30 (dd, 1H), 8.60 (dt, |
| | 1H), 8.65 (t, 1H). |
| Example 443 | 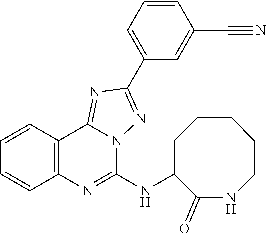 [SMILES: N#Cc1cccc(-c2nc3c4ccccc4nc(NC4CCCCCNC4=O)n3n2)c1] |
| | 3-(5-{[2-oxoazocan-3-yl]amino}[1,2,4]triazolo[1,5-c]quinazolin-2- |
| | yl)benzonitrile |
| | LC-MS (Method 2): Rt = 1.31 min; MS (ESIpos): m/z = 412 [M + H]⁺ |
| | ¹H-NMR (400 MHz, DMSO-d₆): δ [ppm] = 1.49-1.90 (m, 7H), 2.17-2.27 (m, |
| | 1H), 3.20-3.29 (m, 1H), 3.63-3.74 (m, 1H), 5.15 (ddd, 1H), 7.46 (ddd, 1H), |
| | 7.63 (d, 1H), 7.74 (ddd, 1H), 7.79-7.85 (m, 2H), 7.89 (dd, 1H), 8.05 (dt, |
| | 1H), 8.30 (dd, 1H), 8.58 (dt, 1H), 8.63 (t, 1H). |
| Example 444 | 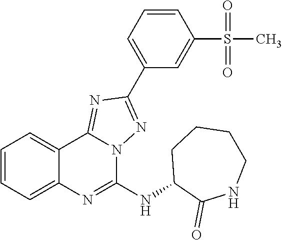 [SMILES: CS(=O)(=O)c1cccc(-c2nc3c4ccccc4nc(N[C@@H]4CCCCNC4=O)n3n2)c1] |
| | (3R)-3-({2-[3-(methanesulfonyl)phenyl][1,2,4]triazolo[1,5-c]quinazolin-5- |
| | yl}amino)azepan-2-one |
| | LC-MS (Method 1): Rt = 1.15 min; MS (ESIpos): m/z = 451 [M + H]⁺ |
| | ¹H-NMR (400 MHz, DMSO-d₆): δ [ppm] = 1.27-1.39 (m, 1H), 1.53-1.65 (m, |
| | 1H), 1.81-1.95 (m, 2H), 1.99-2.07 (m, 1H), 2.28-2.36 (m, 1H), 3.18 (br d, |
| | 1H), 3.34-3.42 (m, 4H), 4.86 (dd, 1H), 7.47 (ddd, 1H), 7.67-7.71 (m, 1H), |
| | 7.76 (ddd, 1H), 7.81 (d, 1H), 7.91 (t, 1H), 8.14 (ddd, 1H), 8.22 (dd, 1H), 8.35 |
| | (dd, 1H), 8.62 (dt, 1H), 8.77 (t, 1H). |
| Example 445 | 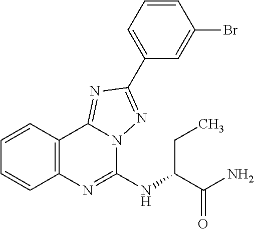 [SMILES: CC[C@@H](Nc1nc2ccccc2c2nc(-c3cccc(Br)c3)nn12)C(N)=O] |
| | (2R)-2-{[2-(3-bromophenyl)[1,2,4]triazolo[1,5-c]quinazolin-5- |
| | yl]amino}butanamide |
| | LC-MS (Method 2): Rt = 1.34 min; MS (ESIpos): m/z = 425 [M + H]⁺ |
| | ¹H-NMR (400 MHz, DMSO-d₆): δ [ppm] = 0.97 (t, 3H), 1.88-2.01 (m, 1H), |
| | 2.01-2.13 (m, 1H), 4.68 (td, 1H), 7.30 (s, 1H), 7.43-7.48 (m, 1H), 7.58 (t, |
| | 1H), 7.63 (d, 1H), 7.67-7.80 (m, 4H), 8.29-8.34 (m, 2H), 8.46 (t, 1H). |
| Example 446 | 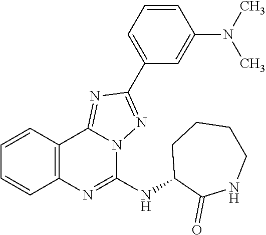 [SMILES: CN(C)c1cccc(-c2nc3c4ccccc4nc(N[C@@H]4CCCCNC4=O)n3n2)c1] |
| | (3R)-3-({2-[3-(dimethylamino)phenyl][1,2,4]triazolo[1,5-c]quinazolin-5- |
| | yl}amino)azepan-2-one |
| | LC-MS (Method 2): Rt = 1.32 min; MS (ESIpos): m/z = 416 [M + H]⁺ |
| | ¹H-NMR (400 MHz, DMSO-d₆): δ [ppm] = 1.26-1.39 (m, 1H), 1.52-1.64 (m, |
| | 1H), 1.81-1.95 (m, 2H), 1.98-2.07 (m, 1H), 2.28-2.35 (m, 1H), 3.02 (s, |
| | 6H), 3.12-3.21 (m, 1H), 3.31-3.41 (m, 1H and water signal), 4.85 (br dd, |
| | 1H), 6.92 (ddd, 1H), 7.38 (t, 1H), 7.45 (ddd, 1H), 7.57-7.62 (m, 2H), 7.65- |
| | 7.68 (m, 1H), 7.71-7.76 (m, 2H), 8.19 (dd, 1H), 8.32 (dd, 1H). |
| Example 447 | 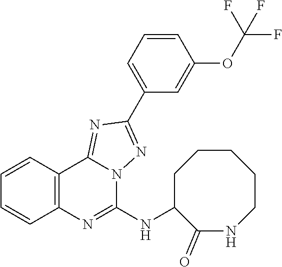 [SMILES: O=C1NCCCCCC1Nc1nc2ccccc2c2nc(-c3cccc(OC(F)(F)F)c3)nn12] |
| | 3-({2-[3-(trifluoromethoxy)phenyl][1,2,4]triazolo[1,5-c]quinazolin-5- |
| | yl}amino)azocan-2-one |
| | LC-MS (Method 2): Rt = 1.51 min; MS (ESIpos): m/z = 471 [M + H]⁺ |
| | ¹H-NMR (400 MHz, DMSO-d₆): δ [ppm] = 1.50-1.89 (m, 7H), 2.18-2.28 (m, |
| | 1H), 3.19-3.27 (m, 1H), 3.62-3.73 (m, 1H), 5.10-5.18 (m, 1H), 7.44-7.49 |
| | (m, 1H), 7.60 (br d, 1H), 7.65 (d, 1H), 7.72-7.80 (m, 3H), 7.87 (br dd, 1H), |
| | 8.19 (s, 1H), 8.29-8.36 (m, 2H). |
| Example 448 | 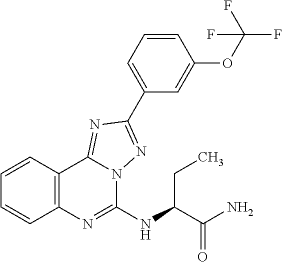 [SMILES: CC[C@H](Nc1nc2ccccc2c2nc(-c3cccc(OC(F)(F)F)c3)nn12)C(N)=O] |
| | (2S)-2-({2-[3-(trifluoromethoxy)phenyl][1,2,4]triazolo[1,5-c]quinazolin-5- |
| | yl}amino)butanamide |
| | LC-MS (Method 2): Rt = 1.35 min; MS (ESIpos): m/z = 431 [M + H]⁺ |
| | ¹H-NMR (400 MHz, DMSO-d₆): δ [ppm] = 0.97 (t, 3H), 1.89-2.01 (m, 1H), |
| | 2.02-2.13 (m, 1H), 4.69 (td, 1H), 7.31 (s, 1H), 7.46 (td, 1H), 7.58-7.62 (m, |
| | 1H), 7.64 (d, 1H), 7.68-7.79 (m, 4H), 8.19-8.22 (m, 1H), 8.32 (dd, 1H), |
| | 8.36 (dt, 1H). |
| Example 449 | 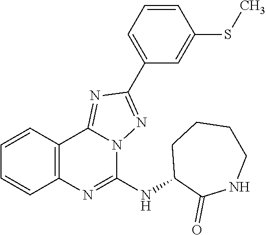 [SMILES: CSc1cccc(-c2nc3c4ccccc4nc(N[C@@H]4CCCCNC4=O)n3n2)c1] |
| | (3R)-3-({2-[3-(methylsulfanyl)phenyl][1,2,4]triazolo[1,5-c]quinazolin-5- |
| | yl}amino)azepan-2-one |
| | LC-MS (Method 2): Rt = 1.39 min; MS (ESIpos): m/z = 419 [M + H]⁺ |
| | ¹H-NMR (400 MHz, DMSO-d₆): δ [ppm] = 1.26-1.38 (m, 1H), 1.53-1.64 (m, |
| | 1H), 1.82-1.94 (m, 2H), 1.98-2.07 (m, 1H), 2.28-2.35 (m, 1H), 2.59 (s, 3H), |
| | 3.12-3.21 (m, 1H), 3.31-3.42 (m, 1H), 4.85 (br dd, 1H), 7.44-7.49 (m, 2H), |
| | 7.53-7.58 (m, 1H), 7.66-7.69 (m, 1H), 7.72-7.76 (m, 1H), 7.78 (d, 1H), 8.06 |
| | (dt, 1H), 8.13 (t, 1H), 8.18-8.23 (m, 1H), 8.32 (dd, 1H). |
| Eample 450 | 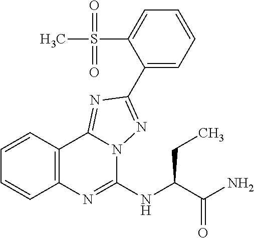 [SMILES: CC[C@H](Nc1nc2ccccc2c2nc(-c3ccccc3S(C)(=O)=O)nn12)C(N)=O] |
| | (2S)-2-({2-[2-(methanesulfonyl)phenyl][1,2,4]triazolo[1,5-c]quinazolin-5- |
| | yl}amino)butanamide |
| | LC-MS (Method 2): Rt = 0.95 min; MS (ESIpos): m/z = 425 [M + H]⁺ |
| | ¹H-NMR (400 MHz, DMSO-d₆): δ [ppm] = 0.95 (t, 3H), 1.86-1.99 (m, 1H), |
| | 2.00-2.11 (m, 1H), 3.71 (s, 3H), 4.68 (td, 1H), 7.30 (s, 1H), 7.47 (ddd, 1H), |
| | 7.67-7.69 (m, 1H), 7.70 (s, 1H), 7.73 (d, 1H), 7.77 (ddd, 1H), 7.85-7.96 |
| | (m, 3H), 8.17-8.21 (m, 1H), 8.27 (dd, 1H). |
| Example 451 | 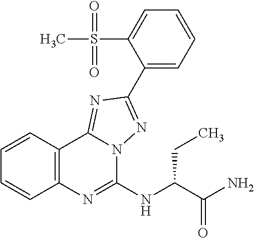 [SMILES: CC[C@@H](Nc1nc2ccccc2c2nc(-c3ccccc3S(C)(=O)=O)nn12)C(N)=O] |
| | (2R)-2-({2-[2-(methanesulfonyl)phenyl][1,2,4]triazolo[1,5-c]quinazolin-5- |
| | yl}amino)butanamide |
| | LC-MS (Method 2): Rt = 0.96 min; MS (ESIpos): m/z = 425 [M + H]⁺ |
| | ¹H-NMR (400 MHz, DMSO-d₆): δ [ppm] = 0.95 (t, 3H), 1.86-1.98 (m, 1H), |
| | 2.00-2.11 (m, 1H), 3.71 (s, 3H), 4.68 (br d, 1H), 7.31 (s, 1H), 7.47 (ddd, |
| | 1H), 7.66-7.71 (m, 2H), 7.72-7.79 (m, 2H), 7.86-7.92 (m, 1H), 7.92-7.96 |
| | (m, 2H), 8.18-8.21 (m, 1H), 8.27 (dd, 1H). |
| Example 452 | 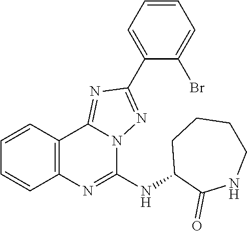 [SMILES: O=C1NCCCC[C@H]1Nc1nc2ccccc2c2nc(-c3ccccc3Br)nn12] |
| | (3R)-3-{[2-(2-bromophenyl)[1,2,4]triazolo[1,5-c]quinazolin-5- |
| | yl]amino}azepan-2-one |
| | LC-MS (Method 1): Rt = 1.32 min; MS (ESIpos): m/z = 451 [M + H]⁺ |
| | ¹H-NMR (400 MHz, DMSO-d₆): δ [ppm] = 1.25-1.38 (m, 1H), 1.49-1.61 (m, |
| | 1H), 1.80-1.96 (m, 2H), 1.97-2.07 (m, 1H), 2.31-2.38 (m, 1H), 3.11-3.20 |
| | (m, 1H), 3.31-3.41 (m, 1H), 4.83 (br dd, 1H), 7.44-7.49 (m, 1H), 7.47 (ddd, |
| | 1H), 7.60 (td, 1H), 7.68-7.71 (m, 1H), 7.74-7.79 (m, 2H), 7.87 (dd, 1H), |
| | 7.95 (dd, 1H), 8.21 (br dd, 1H), 8.28 (dd, 1H). |
| Example 453 | 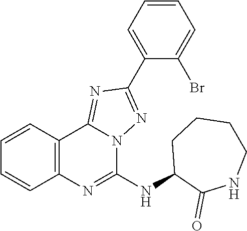 [SMILES: O=C1NCCCC[C@@H]1Nc1nc2ccccc2c2nc(-c3ccccc3Br)nn12] |
| | (3S)-3-{[2-(2-bromophenyl)[1,2,4]triazolo[1,5-c]quinazolin-5- |
| | yl]amino}azepan-2-one |
| | LC-MS (Method 1): Rt = 1.54 min; MS (ESIpos): m/z = 451 [M + H]⁺ |
| | ¹H-NMR (400 MHz, DMSO-d₆): δ [ppm] = 1.25-1.38 (m, 1H), 1.49-1.61 (m, |
| | 1H), 1.81-1.95 (m, 2H), 1.98-2.07 (m, 1H), 2.31-2.39 (m, 1H), 3.11-3.20 |
| | (m, 1H), 3.32-3.41 (m, 1H and water signal), 4.83 (dd, 1H), 7.45-7.54 (m, |
| | 2H), 7.60 (td, 1H), 7.68-7.72 (m, 1H), 7.74-7.79 (m, 2H), 7.87 (dd, 1H), |
| | 7.95 (dd, 1H), 8.21 (dd, 1H), 8.29 (dd, 1H). |
| Example 454 | 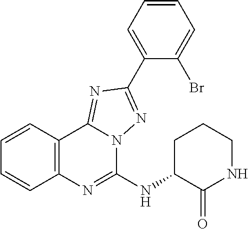 [SMILES: O=C1NCCC[C@H]1Nc1nc2ccccc2c2nc(-c3ccccc3Br)nn12] |
| | (3R)-3-{[2-(2-bromophenyl)[1,2,4]triazolo[1,5-c]quinazolin-5- |
| | yl]amino}piperidin-2-one |
| | LC-MS (Method 1): Rt = 1.17 min; MS (ESIpos): m/z = 437 [M + H]⁺ |
| | ¹H-NMR (400 MHz, DMSO-d₆): δ [ppm] = 1.85-1.97 (m, 2H), 2.10 (qd, 1H), |
| | 2.26-2.35 (m, 1H), 3.18-3.30 (m, 2H), 4.68 (dt, 1H), 7.44 (ddd, 1H), 7.49- |
| | 7.54 (m, 1H), 7.59 (dt, 1H), 7.64-7.68 (m, 1H), 7.74 (ddd, 1H), 7.78 (br s, |
| | 1H), 7.86 (dd, 1H), 7.91 (dd, 1H), 8.19 (d, 1H), 8.27 (dd, 1H). |
| Example 455 | 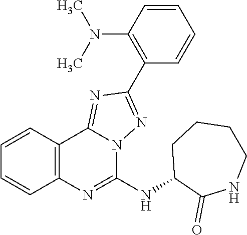 [SMILES: CN(C)c1ccccc1-c1nc2c3ccccc3nc(N[C@@H]3CCCCNC3=O)n2n1] |
| | (3R)-3-({2-[2-(dimethylamino)phenyl][1,2,4]triazolo[1,5-c]quinazolin-5- |
| | yl}amino)azepan-2-one |
| | LC-MS (Method 2): Rt = 1.24 min; MS (ESIpos): m/z = 416 [M + H]⁺ |
| | ¹H-NMR (400 MHz, DMSO-d₆): δ [ppm] = 1.25-1.38 (m, 1H), 1.49-1.61 (m, |
| | 1H), 1.81-1.96 (m, 2H), 1.98-2.07 (m, 1H), 2.31-2.39 (m, 1H), 2.67 (s, |
| | 6H), 3.11-3.21 (m, 1H), 3.32-3.41 (m, 1H and water signal), 4.82 (dd, 1H), |
| | 7.02 (td, 1H), 7.14 (dd, 1H), 7.41-7.47 (m, 2H), 7.66-7.69 (m, 1H), 7.70- |
| | 7.76 (m, 3H), 8.20 (dd, 1H), 8.28 (dd, 1H). |
| Example 456 | 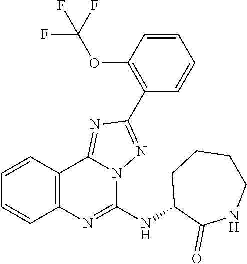 [SMILES: O=C1NCCCC[C@H]1Nc1nc2ccccc2c2nc(-c3ccccc3OC(F)(F)F)nn12] |
| | (3R)-3-({2-[2-(trifluoromethoxy)phenyl][1,2,4]triazolo[1,5-c]quinazolin-5- |
| | yl}amino)azepan-2-one |
| | LC-MS (Method 2): Rt = 1.45 min; MS (ESIpos): m/z = 457 [M + H]⁺ |
| | ¹H-NMR (400 MHz, DMSO-d₆): δ [ppm] = 1.25-1.39 (m, 1H), 1.47-1.59 (m, |
| | 1H), 1.81-1.96 (m, 2H), 1.98-2.08 (m, 1H), 2.33-2.40 (m, 1H), 3.11-3.21 |
| | (m, 1H), 4.82 (br dd, 1H), 7.46-7.50 (m, 1H), 7.59-7.79 (m, 6H), 8.20 (dd, |
| | 1H), 8.29 (dd, 1H), 8.38 (dd, 1H). |
| Example 457 | 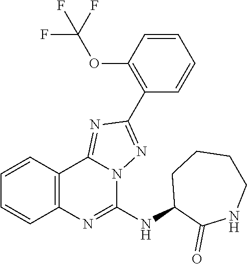 [SMILES: O=C1NCCCC[C@@H]1Nc1nc2ccccc2c2nc(-c3ccccc3OC(F)(F)F)nn12] |
| | (3S)-3-({2-[2-(trifluoromethoxy)phenyl][1,2,4]triazolo[1,5-c]quinazolin-5- |
| | yl}amino)azepan-2-one |
| | LC-MS (Method 2): Rt = 1.43 min; MS (ESIpos): m/z = 457 [M + H]⁺ |
| | ¹H-NMR (400 MHz, DMSO-d₆): δ [ppm] = 1.26-1.39 (m, 1H), 1.47-1.59 (m, |
| | 1H), 1.81-1.96 (m, 2H), 1.99-2.07 (m, 1H), 2.33-2.40 (m, 1H), 3.12-3.21 |
| | (m, 1H), 3.34-3.41 (m, 1H), 4.82 (br dd, 1H), 7.48 (ddd, 1H), 7.60-7.79 (m, |
| | 6H), 8.20 (dd, 1H), 8.29 (dd, 1H), 8.38 (dd, 1H). |
| Example 458 | 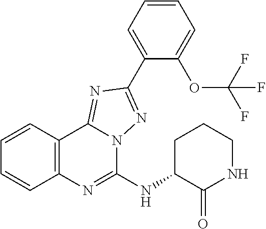 [SMILES: O=C1NCCC[C@H]1Nc1nc2ccccc2c2nc(-c3ccccc3OC(F)(F)F)nn12] |
| | (3R)-3-({2-[2-(trifluoromethoxy)phenyl][1,2,4]triazolo[1,5-c]quinazolin-5- |
| | yl}amino)piperidin-2-one |
| | LC-MS (Method 2): Rt = 1.29 min; MS (ESIpos): m/z = 443 [M + H]⁺ |
| | ¹H-NMR (400 MHz, DMSO-d₆): δ [ppm] = 1.87-2.07 (m, 3H), 2.39-2.46 (m, |
| | 1H), 3.20-3.30 (m, 2H), 4.62-4.69 (m, 1H), 7.46 (ddd, 1H), 7.59-7.69 (m, |
| | 3H), 7.70-7.78 (m, 2H), 7.84 (br s, 1H), 8.00 (d, 1H), 8.26-8.32 (m, 2H). |
| Example 459 | 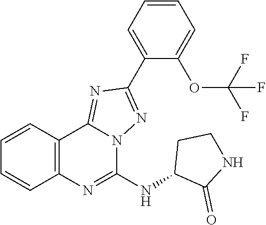 [SMILES: O=C1NCC[C@H]1Nc1nc2ccccc2c2nc(-c3ccccc3OC(F)(F)F)nn12] |
| | (3R)-3-({2-[2-(trifluoromethoxy)phenyl][1,2,4]triazolo[1,5-c]quinazolin-5- |
| | yl}amino)pyrrolidin-2-one |
| | LC-MS (Method 2): Rt = 1.21 min; MS (ESIpos): m/z = 429 [M + H]⁺ |
| | ¹H-NMR (400 MHz, DMSO-d₆): δ [ppm] = 2.23-2.34 (m, 1H), 2.53-2.61 (m, |
| | 1H), 3.35 (d, 1H), 4.89 (dt, 1H), 7.47 (ddd, 1H), 7.60-7.68 (m, 3H), 7.70- |
| | 7.78 (m, 2H), 8.02 (s, 1H), 8.11 (d, 1H), 8.26-8.29 (m, 2H). |
| Example 460 | 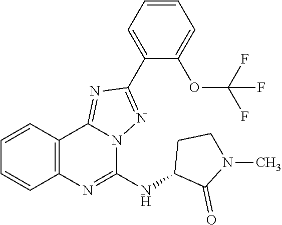 [SMILES: CN1CC[C@@H](Nc2nc3ccccc3c3nc(-c4ccccc4OC(F)(F)F)nn23)C1=O] |
| | (3R)-1-methyl-3-({2-[2-(trifluoromethoxy)phenyl][1,2,4]triazolo[1,5- |
| | c]quinazolin-5-yl}amino)pyrrolidin-2-one |
| | LC-MS (Method 2): Rt = 1.29 min; MS (ESIpos): m/z = 443 [M + H]⁺ |
| | ¹H-NMR (400 MHz, DMSO-d₆): δ [ppm] = 2.21 (dq, 1H), 2.83 (s, 3H), 3.44 |
| | (dd, 2H), 4.93 (q, 1H), 7.47 (ddd, 1H), 7.60-7.67 (m, 3H), 7.70-7.78 (m, |
| | 2H), 8.24-8.29 (m, 3H). |
| Example 461 | 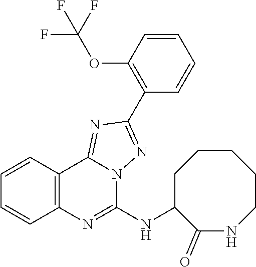 [SMILES: O=C1NCCCCCC1Nc1nc2ccccc2c2nc(-c3ccccc3OC(F)(F)F)nn12] |
| | 3-({2-[2-(trifluoromethoxy)phenyl][1,2,4]triazolo[1,5-c]quinazolin-5- |
| | yl}amino)azocan-2-one |
| | ¹H-NMR (400 MHz, DMSO-d₆): δ [ppm] = 1.47-1.76 (m, 7H), 2.31-2.38 (m, |
| | 1H), 3.15-3.24 (m, 1H), 3.58-3.69 (m, 1H), 5.07-5.14 (m, 1H), 7.45-7.50 |
| | (m, 1H), 7.54 (d, 1H), 7.59-7.79 (m, 5H), 7.90 (dd, 1H), 8.28 (dd, 1H), 8.37 |
| | (dd, 1H). |
| Example 462 | 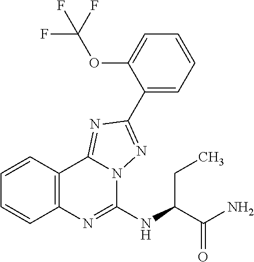 [SMILES: CC[C@H](Nc1nc2ccccc2c2nc(-c3ccccc3OC(F)(F)F)nn12)C(N)=O] |
| | (2S)-2-({2-[2-(trifluoromethoxy)phenyl][1,2,4]triazolo[1,5-c]quinazolin-5- |
| | yl}amino)butanamide |
| | LC-MS (Method 2): Rt = 1.25 min; MS (ESIpos): m/z = 431 [M + H]⁺ |
| | ¹H-NMR (400 MHz, DMSO-d₆): δ [ppm] = 0.94 (t, 3H), 1.86-1.98 (m, 1H), |
| | 2.02-2.14 (m, 1H), 4.73 (td, 1H), 7.35 (s, 1H), 7.40 (d, 1H), 7.45-7.50 (m, |
| | 1H), 7.60-7.68 (m, 3H), 7.72 (dd, 1H), 7.73-7.78 (m, 2H), 8.29 (dd, 1H), |
| | 8.37 (dd, 1H). |
| Example 463 | 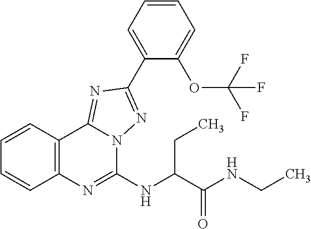 [SMILES: CCNC(=O)C(CC)Nc1nc2ccccc2c2nc(-c3ccccc3OC(F)(F)F)nn12] |
| | N-ethyl-2-({2-[2-(trifluoromethoxy)phenyl][1,2,4]triazolo[1,5- |
| | c]quinazolin-5-yl}amino)butanamide |
| | LC-MS (Method 2): Rt = 1.49 min; MS (ESIpos): m/z = 459 [M + H]⁺ |
| | ¹H-NMR (400 MHz, DMSO-d₆): δ [ppm] = 0.93 (t, 3H), 1.04 (t, 3H), 1.84-1.96 |
| | (m, 1H), 1.96-2.08 (m, 1H), 3.11-3.19 (m, 2H), 4.68-4.75 (m, 1H), 7.40 |
| | (d, 1H), 7.47 (ddd, 1H), 7.60-7.68 (m, 3H), 7.70-7.78 (m, 2H), 8.26-8.31 |
| | (m, 2H), 8.38 (dd, 1H). |
| Example 464 | 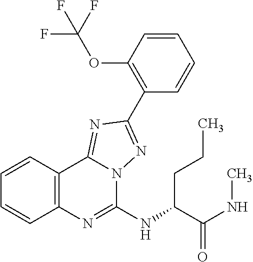 [SMILES: CCC[C@@H](Nc1nc2ccccc2c2nc(-c3ccccc3OC(F)(F)F)nn12)C(=O)NC] |
| | N-methyl-N²-{2-[2-(trifluoromethoxy)phenyl][1,2,4]triazolo[1,5- |
| | c]quinazolin-5-yl}-D-norvalinamide |
| | LC-MS (Method 2): Rt = 1.51 min; MS (ESIpos): m/z = 459 [M + H]⁺ |
| | ¹H-NMR (400 MHz, DMSO-d₆): δ [ppm] = 0.91 (t, 3H), 1.31-1.46 (m, 2H), |
| | 1.81-2.00 (m, 2H), 2.64 (d, 3H), 4.77 (td, 1H), 7.45-7.51 (m, 2H), 7.60- |
| | 7.68 (m, 3H), 7.70-7.78 (m, 2H), 8.19 (q, 1H), 8.28 (dd, 1H), 8.37 (dd, 1H). |
| Example 465 | 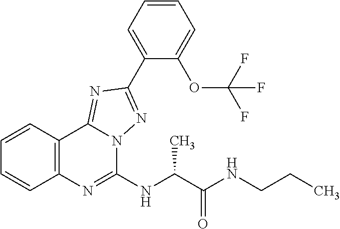 [SMILES: CCCNC(=O)[C@@H](C)Nc1nc2ccccc2c2nc(-c3ccccc3OC(F)(F)F)nn12] |
| | N-propyl-N²-{2-[2-(trifluoromethoxy)phenyl][1,2,4]triazolo[1,5- |
| | c]quinazolin-5-yl}-D-alaninamide |
| | LC-MS (Method 2): Rt = 1.41 min; MS (ESIpos): m/z = 459 [M + H]⁺ |
| | ¹H-NMR (400 MHz, DMSO-d₆): δ [ppm] = 0.85 (t, 3H), 1.39-1.49 (m, 2H), |
| | 1.52 (d, 3H), 3.09 (q, 2H), 4.76 (quin, 1H), 7.47 (t, 1H), 7.57 (d, 1H), 7.60- |
| | 7.68 (m, 3H), 7.70-7.78 (m, 2H), 8.23 (br t, 1H), 8.28 (d, 1H), 8.35 (dd, 1H). |
| Example 466 | 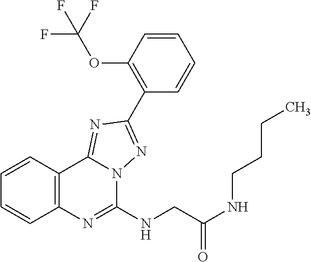 [SMILES: CCCCNC(=O)CNc1nc2ccccc2c2nc(-c3ccccc3OC(F)(F)F)nn12] |
| | N-butyl-N²-{2-[2-(trifluoromethoxy)phenyl][1,2,4]triazolo[1,5- |
| | c]quinazolin-5-yl}glycinamide |
| | LC-MS (Method 2): Rt = 1.50 min; MS (ESIpos): m/z = 459 [M + H]⁺ |
| | ¹H-NMR (400 MHz, DMSO-d₆): δ [ppm] = 0.85 (t, 3H), 1.23-1.33 (m, 2H), |
| | 1.35-1.44 (m, 2H), 3.10 (q, 2H), 4.17 (d, 2H), 7.46 (ddd, 1H), 7.60-7.68 |
| | (m, 3H), 7.70-7.78 (m, 2H), 8.00 (t, 1H), 8.06 (t, 1H), 8.29 (td, 2H). |
| Example 467 | 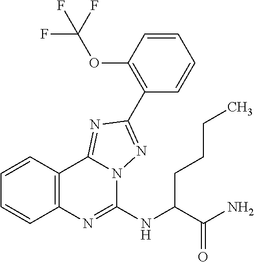 [SMILES: CCCCC(Nc1nc2ccccc2c2nc(-c3ccccc3OC(F)(F)F)nn12)C(N)=O] |
| | N²-{2-[2-(trifluoromethoxy)phenyl][1,2,4]triazolo[1,5-c]quinazolin-5- |
| | yl}norleucinamide |
| | LC-MS (Method 2): Rt = 1.43 min; MS (ESIpos): m/z = 459 [M + H]⁺ |
| | ¹H-NMR (400 MHz, DMSO-d₆): δ [ppm] = 0.85 (t, 3H), 1.27-1.41 (m, 4H), |
| | 1.83-1.94 (m, 1H), 1.96-2.07 (m, 1H), 4.75 (td, 1H), 7.32 (s, 1H), 7.41 (d, |
| | 1H), 7.45-7.50 (m, 1H), 7.60-7.68 (m, 3H), 7.70-7.78 (m, 3H), 8.28 (dd, |
| | 1H), 8.37 (dd, 1H). |
| Example 468 | 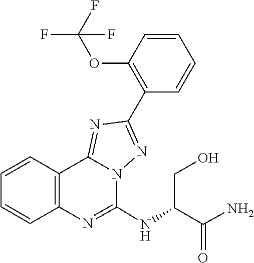 [SMILES: NC(=O)[C@@H](CO)Nc1nc2ccccc2c2nc(-c3ccccc3OC(F)(F)F)nn12] |
| | N²-{2-[2-(trifluoromethoxy)phenyl][1,2,4]triazolo[1,5-c]quinazolin-5-yl}- |
| | D-serinamide |
| | LC-MS (Method 2): Rt = 1.08 min; MS (ESIpos): m/z = 433 [M + H]⁺ |
| | ¹H-NMR (400 MHz, DMSO-d₆): δ [ppm] = 3.95 (t, 2H), 4.67-4.73 (m, 1H), |
| | 5.17 (t, 1H), 7.33 (s, 1H), 7.42 (d, 1H), 7.48 (s, 1H), 7.59-7.79 (m, 6H), 8.29 |
| | (dd, 1H), 8.37 (dd, 1H). |
| Example 469 | 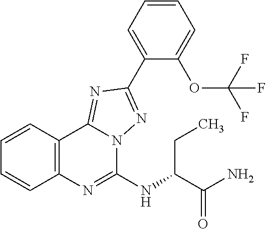 [SMILES: CC[C@@H](Nc1nc2ccccc2c2nc(-c3ccccc3OC(F)(F)F)nn12)C(N)=O] |
| | (2R)-2-({2-[2-(trifluoromethoxy)phenyl][1,2,4]triazolo[1,5-c]quinazolin-5- |
| | yl}amino)butanamide |
| | LC-MS (Method 2): Rt = 1.27 min; MS (ESIpos): m/z = 431 [M + H]⁺ |
| | ¹H-NMR (400 MHz, DMSO-d₆): δ [ppm] = 0.94 (t, 3H), 1.85-1.98 (m, 1H), |
| | 2.02-2.14 (m, 1H), 4.69-4.76 (m, 1H), 7.35 (s, 1H), 7.41 (d, 1H), 7.47 |
| | (ddd, 1H), 7.60-7.68 (m, 3H), 7.70-7.78 (m, 3H), 8.29 (dd, 1H), 8.37 (dd, |
| | 1H). |
| Example 470 | 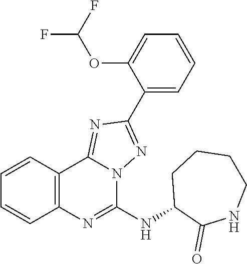 [SMILES: O=C1NCCCC[C@H]1Nc1nc2ccccc2c2nc(-c3ccccc3OC(F)F)nn12] |
| | (3R)-3-({2-[2-(difluoromethoxy)phenyl][1,2,4]triazolo[1,5-c]quinazolin-5- |
| | yl}amino)azepan-2-one |
| | LC-MS (Method 2): Rt = 1.30 min; MS (ESIpos): m/z = 439 [M + H]⁺ |
| | ¹H-NMR (400 MHz, DMSO-d₆): δ [ppm] = 1.26-1.39 (m, 1H), 1.49-1.61 (m, |
| | 1H), 1.81-1.96 (m, 2H), 1.98-2.07 (m, 1H), 2.31-2.38 (m, 1H), 3.12-3.21 |
| | (m, 1H), 3.35-3.41 (m, 1H), 4.83 (br dd, 1H), 7.28 (t, 1H), 7.42 (dd, 1H), |
| | 7.45-7.54 (m, 2H), 7.65 (ddd, 1H), 7.68-7.71 (m, 1H), 7.73-7.79 (m, 2H), |
| | 8.19 (dd, 1H), 8.23 (dd, 1H), 8.29 (dd, 1H). |
| Example 471 | 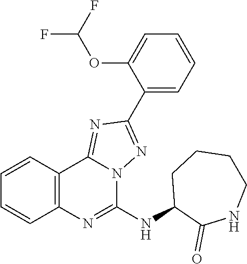 [SMILES: O=C1NCCCC[C@@H]1Nc1nc2ccccc2c2nc(-c3ccccc3OC(F)F)nn12] |
| | (3S)-3-({2-[2-(difluoromethoxy)phenyl][1,2,4]triazolo[1,5-c]quinazolin-5- |
| | yl}amino)azepan-2-one |
| | LC-MS (Method 2): Rt = 1.31 min; MS (ESIpos): m/z = 439 [M + H]⁺ |
| | ¹H-NMR (400 MHz, DMSO-d₆): δ [ppm] = 1.26-1.39 (m, 1H), 1.49-1.61 (m, |
| | 1H), 1.81-1.96 (m, 2H), 1.98-2.07 (m, 1H), 2.30-2.38 (m, 1H), 3.12-3.21 |
| | (m, 1H), 3.36-3.41 (m, 1H), 4.83 (br dd, 1H), 7.28 (t, 1H), 7.42 (d, 1H), 7.45- |
| | 7.54 (m, 2H), 7.65 (ddd, 1H), 7.68-7.71 (m, 1H), 7.73-7.79 (m, 2H), 8.19 |
| | (dd, 1H), 8.23 (dd, 1H), 8.29 (dd, 1H). |
| Example 472 | 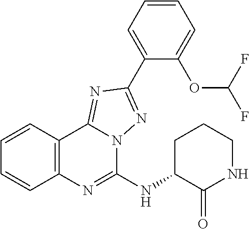 [SMILES: O=C1NCCC[C@H]1Nc1nc2ccccc2c2nc(-c3ccccc3OC(F)F)nn12] |
| | (3R)-3-({2-[2-(difluoromethoxy)phenyl][1,2,4]triazolo[1,5-c]quinazolin-5- |
| | yl}amino)piperidin-2-one |
| | LC-MS (Method 2): Rt = 1.18 min; MS (ESIpos): m/z = 425 [M + H]⁺ |
| | ¹H-NMR (400 MHz, DMSO-d₆): δ [ppm] = 1.87-1.97 (m, 2H), 2.00-2.12 (m, |
| | 1H), 2.31-2.41 (m, 1H), 3.20-3.31 (m, 2H), 4.68 (dt, 1H), 7.32 (t, 1H), 7.41- |
| | 7.53 (m, 3H), 7.62-7.68 (m, 2H), 7.74 (ddd, 1H), 7.82 (br s, 1H), 8.03 (d, |
| | 1H), 8.17 (dd, 1H), 8.28 (dd, 1H). |
| Example 473 | 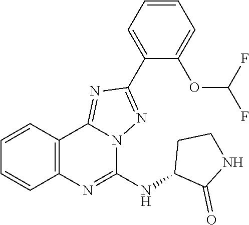 [SMILES: O=C1NCC[C@H]1Nc1nc2ccccc2c2nc(-c3ccccc3OC(F)F)nn12] |
| | (3R)-3-({2-[2-(difluoromethoxy)phenyl][1,2,4]triazolo[1,5-c]quinazolin-5- |
| | yl}amino)pyrrolidine-2-one |
| | LC-MS (Method 2): Rt = 1.11 min; MS (ESIpos): m/z = 411 [M + H]⁺ |
| | ¹H-NMR (400 MHz, DMSO-d₆): δ [ppm] = 2.24-2.35 (m, 1H), 2.53-2.58 |
| | (m, 1H), 3.35 (br d, 1H), 4.88-4.96 (m, 1H), 7.33 (s, 1H), 7.41-7.53 (m, |
| | 3H), 7.63-7.68 (m, 2H), 7.75 (ddd, 1H), 8.01 (s, 1H), 8.12-8.17 (m, 2H), |
| | 8.28 (dd, 1H). |
| Example 474 | 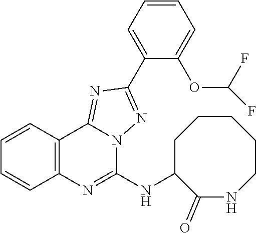 [SMILES: O=C1NCCCCCC1Nc1nc2ccccc2c2nc(-c3ccccc3OC(F)F)nn12] |
| | 3-({2-[2-(difluoromethoxy)phenyl][1,2,4]triazolo[1,5-c]quinazolin-5- |
| | yl}amino)azocan-2-one |
| | LC-MS (Method 2): Rt = 1.35 min; MS (ESIpos): m/z = 453 [M + H]⁺ |
| | ¹H-NMR (400 MHz, DMSO-d₆): δ [ppm] = 1.50-1.77 (m, 7H), 2.24-2.34 (m, |
| | 1H), 3.16-3.25 (m, 1H), 3.59-3.71 (m, 1H), 5.08-5.16 (m, 1H), 7.30 (t, |
| | 1H), 7.42 (d, 1H), 7.45-7.56 (m, 3H), 7.62-7.69 (m, 2H), 7.76 (ddd, 1H), |
| | 7.88 (dd, 1H), 8.23 (dd, 1H), 8.28 (dd, 1H). |
| Example 475 | 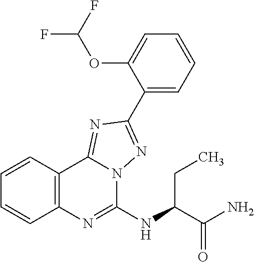 [SMILES: CC[C@H](Nc1nc2ccccc2c2nc(-c3ccccc3OC(F)F)nn12)C(N)=O] |
| | (2S)-2-({2-[2-(difluoromethoxy)phenyl][1,2,4]triazolo[1,5-c]quinazolin-5- |
| | yl}amino)butanamaide |
| | LC-MS (Method 2): Rt = 1.18 min; MS (ESIpos): m/z = 413 [M + H]⁺ |
| | ¹H-NMR (400 MHz, DMSO-d₆): δ [ppm] = 0.95 (t, 3H), 1.86-1.92 (m, 1H), |
| | 2.01-2.12 (m, 1H), 4.71 (td, 1H), 7.30 (t, 1H), 7.32 (s, 1H), 7.41-7.45 (m, |
| | 2H), 7.46-7.49 (m, 1H), 7.52 (ddd, 1H), 7.63-7.68 (m, 2H), 7.71-7.78i (m, |
| | 2H), 8.23 (dd, 1H), 8.29 (dd, 1H). |
| Example 476 | 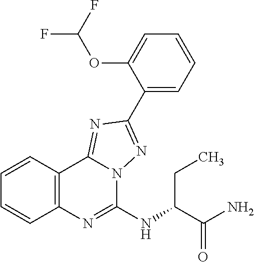 [SMILES: CC[C@@H](Nc1nc2ccccc2c2nc(-c3ccccc3OC(F)F)nn12)C(N)=O] |
| | (2R)-2-({2-[2-(difluoromethoxy)phenyl][1,2,4]triazolo[1,5-c]quinazolin-5- |
| | yl}amino)butanamide |
| | LC-MS (Method 2): Rt = 1.16 min; MS (ESIpos): m/z = 413 [M + H]⁺ |
| | ¹H-NMR (400 MHz, DMSO-d₆): δ [ppm] = 0.95 (t, 3H), 1.86-1.98 (m, 1H), |
| | 2.00-2.12 (m, 1H), 4.71 (td, 1H), 7.30 (t, 1H), 7.32 (s, 1H), 7.41-7.46 (m, |
| | 2H), 7.48 (d, 1H), 7.52 (td, 1H), 7.63-7.68 (m, 2H), 7.71-7.78 (m, 2H), |
| | 8.23 (dd, 1H), 8.29 (dd, 1H). |
| Example 477 | 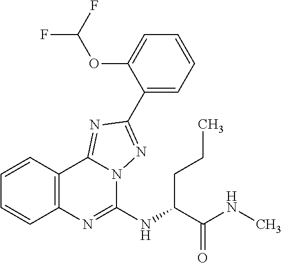 [SMILES: CCC[C@@H](Nc1nc2ccccc2c2nc(-c3ccccc3OC(F)F)nn12)C(=O)NC] |
| | N²⁻{2-[2-(difluoromethoxy)phenyl][1,2,4]triazolo[1,5-c]quinazolin-5-yl}- |
| | N-methyl-D-norvalinamide |
| | LC-MS (Method 2): Rt = 1.28 min; MS (ESIpos): m/z = 441 [M + H]⁺ |
| | ¹H-NMR (400 MHz, DMSO-d₆): δ [ppm] = 0.91 (t, 3H), 1.32-1.48 (m, 2H), |
| | 1.81-1.99 (m, 2H), 2.63 (d, 3H), 4.77 (td, 1H), 7.32 (t, 1H), 7.41-7.49 (m, |
| | 2H), 7.49-7.56 (m, 2H), 7.63-7.68 (m, 2H), 7.74 (ddd, 1H), 8.16 (q, 1H), |
| | 8.22 (dd, 1H), 8.29 (dd, 1H). |
| Example 478 | 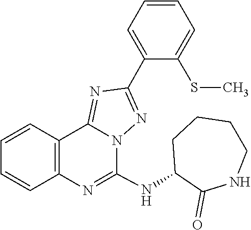 [SMILES: CSc1ccccc1-c1nc2c3ccccc3nc(N[C@@H]3CCCCNC3=O)n2n1] |
| | (3R)-3-({2-[2-(methylsulfanyl)phenyl][1,2,4]triazolo[1,5-c]quinazolin-5- |
| | yl}amino)azepan-2-one |
| | LC-MS (Method 2): Rₜ = 1.32 min; MS (ESIpos): m/z = 419 [M + H]⁺ |
| | ¹H-NMR (400 MHz, DMSO-d₆): δ [ppm] = 1.25-1.39 (m, 1H), 1.49-1.61 (m, |
| | 1H), 1.80-1.96 (m, 2H), 1.99-2.07 (m, 1H), 2.31-2.40 (m, 1H), 3.10-3.21 |
| | (m, 1H), 4.83 (br dd, 1H), 7.34 (td, 1H), 7.44-7.51 (m, 2H), 7.53-7.58 (m, |
| | 1H), 7.67-7.79 (m, 3H), 8.09 (dd, 1H), 8.20 (br dd, 1H), 8.29 (dd, 1H). |
| Example 479 | 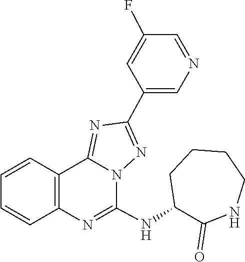 [SMILES: O=C1NCCCC[C@H]1Nc1nc2ccccc2c2nc(-c3cncc(F)c3)nn12] |
| | (3R)-3-{[2-(5-fluoropyridin-3-yl)[1,2,4]triazolo[1,5-c]quinazolin-5- |
| | yl]amino}azepan-2-one |
| | LC-MS (Method 2): Rt = 1.16 min; MS (ESIpos): m/z = 392 [M + H]⁺ |
| | ¹H-NMR (400 MHz, DMSO-d₆): δ [ppm] = 1.26-1.39 (m, 1H), 1.53-1.64 (m, |
| | 1H), 1.81-1.95 (m, 2H), 1.98-2.08 (m, 1H), 2.27-2.35 (m, 1H), 3.12-3.22 |
| | (m, 1H), 3.31-3.41 (m, 1H and water signal), 4.82-4.88 (m, 1H), 7.48 (ddd, |
| | 1H), 7.67-7.70 (m, 1H), 7.74-7.81 (m, 2H), 8.21 (dd, 1H), 8.32 (dd, 1H), |
| | 8.42 (ddd, 1H), 8.80 (d, 1H), 9.30 (t, 1H). |
| Example 480 | 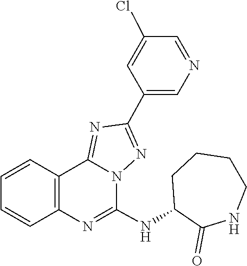 [SMILES: O=C1NCCCC[C@H]1Nc1nc2ccccc2c2nc(-c3cncc(Cl)c3)nn12] |
| | (3R)-3-{[2-(5-chloropyridin-3-yl)[1,2,4]triazolo[1,5-c]quinazolin-5- |
| | yl]amino}azepan-2-one |
| | LC-MS (Method 2): Rt = 1.31 min; MS (ESIpos): m/z = 408 [M + H]⁺ |
| | ¹H-NMR (400 MHz, DMSO-d₆): δ [ppm] = 1.26-1.39 (m, 1H), 1.53-1.65 (m, |
| | 1H), 1.80-1.95 (m, 2H), 1.98-2.08 (m, 1H), 2.26-2.35 (m, 1H), 3.12-3.22 |
| | (m, 1H), 4.82-4.88 (m, 1H), 7.48 (ddd, 1H), 7.66-7.69 (m, 1H), 7.76 (ddd, |
| | 1H), 7.81 (d, 1H), 8.21 (dd, 1H), 8.32 (dd, 1H), 8.61 (dd, 1H), 8.84 (d, 1H), |
| | 9.35 (d, 1H). |
| Example 481 | 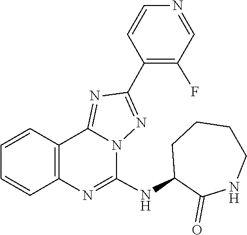 [SMILES: O=C1NCCCC[C@@H]1Nc1nc2ccccc2c2nc(-c3ccncc3F)nn12] |
| | (3S)-3-{[2-(3-fluoropyridin-4-yl)[1,2,4]triazolo[1,5-c]quinazolin-5- |
| | yl]amino}azepan-2-one |
| | LC-MS (Method 2): Rt = 1.16 min; MS (ESIpos): m/z = 392 [M + H]⁺ |
| | ¹H-NMR (400 MHz, DMSO-d₆): δ [ppm] = 1.26-1.39 (m, 1H), 1.50-1.62 (m, |
| | 1H), 1.82-1.96 (m, 2H), 1.99-2.08 (m, 1H), 2.30-2.38 (m, 1H), 3.12-3.21 |
| | (m, 1H), 4.84 (br dd, 1H), 7.49 (ddd, 1H), 7.69-7.72 (m, 1H), 7.75-7.81 (m, |
| | 2H), 8.21-8.28 (m, 2H), 8.32 (dd, 1H), 8.67 (d, 1H), 8.85 (d, 1H). |
| Example 482 | 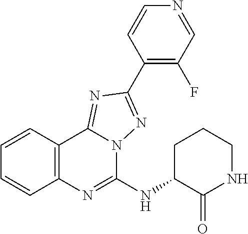 [SMILES: O=C1NCCC[C@H]1Nc1nc2ccccc2c2nc(-c3ccncc3F)nn12] |
| | (3R)-3-{[2-(3-fluoropyridin-4-yl)[1,2,4]triazolo[1,5-c]quinazolin-5- |
| | yl]amino}piperidin-2-one |
| | LC-MS (Method 2): Rt = 0.97 min; MS (ESIpos): m/z = 378 [M + H]⁺ |
| | ¹H-NMR (400 MHz, DMSO-d₆): δ [ppm] = 1.85-1.97 (m, 2H), 2.04-2.16 (m, |
| | 1H), 2.27-2.35 (m, 1H), 3.19-3.29 (m, 2H), 4.71 (dt, 1H), 7.44-7.49 (m, |
| | 1H), 7.65-7.68 (m, 1H), 7.74-7.78 (m, 1H), 7.82 (br s, 1H), 8.22 (d, 1H), |
| | 8.26 (dd, 1H), 8.31 (dd, 1H), 8.68 (d, 1H), 8.86 (d, 1H). |
| Example 483 | 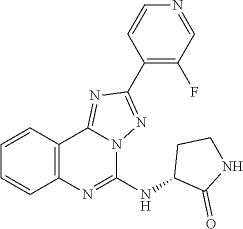 [SMILES: O=C1NCC[C@H]1Nc1nc2ccccc2c2nc(-c3ccncc3F)nn12] |
| | (3R)-3-{[2-(3-fluoropyridin-4-yl)[1,2,4]triazolo[1,5-c]quinazolin-5- |
| | yl]amino}pyrrolidin-2-one |
| | LC-MS (Method 2): Rt = 0.89 min; MS (ESIpos): m/z = 364 [M + H]⁺ |
| | ¹H-NMR (400 MHz, DMSO-d₆): δ [ppm] = 2.26-2.38 (m, 1H), 4.91-4.99 (m, |
| | 1H), 7.47 (t, 1H), 7.65 (d, 1H), 7.74-7.79 (m, 1H), 8.01 (s, 1H), 8.26 (t, 1H), |
| | 8.31 (d, 1H), 8.36 (d, 1H), 8.68 (br d, 1H), 8.86 (br d, 1H). |
| Example 484 | 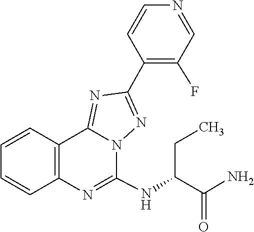 [SMILES: CC[C@@H](Nc1nc2ccccc2c2nc(-c3ccncc3F)nn12)C(N)=O] |
| | (2R)-2-{[2-(3-fluoropyridin-4-yl)[1,2,4]triazolo[1,5-c]quinazolin-5- |
| | yl]amino}butanamide |
| | LC-MS (Method 2): Rt = 0.94 min; MS (ESIpos): m/z = 366 [M + H]⁺ |
| | ¹H-NMR (400 MHz, DMSO-d₆): δ [ppm] = 0.95 (t, 3H), 1.88-2.01 (m, 1H), |
| | 2.03-2.15 (m, 1H), 4.68-4.74 (m, 1H), 7.35 (br s, 1H), 7.48 (t, 1H), 7.60 (br |
| | d, 1H), 7.66 (d, 1H), 7.71-7.79 (m, 2H), 8.27 (br t, 1H), 8.31 (d, 1H), 8.68 |
| | (br d, 1H), 8.86 (br s, 1H). |
| Example 485 | 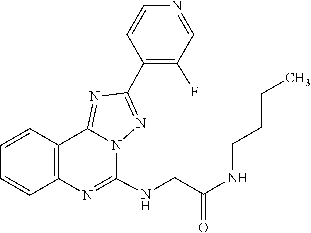 [SMILES: CCCCNC(=O)CNc1nc2ccccc2c2nc(-c3ccncc3F)nn12] |
| | N-butyl-N²-[2-(3-fluoropyridin-4-yl)[1,2,4]triazolo[1,5-c]quinazolin-5- |
| | yl]glycinamide |
| | LC-MS (Method 2): Rt = 1.07 min; MS (ESIpos): m/z = 394 [M + H]⁺ |
| | ¹H-NMR (400 MHz, DMSO-d₆): δ [ppm] = 0.84 (t, 3H), 1.22-1.32 (m, 2H), |
| | 1.34-1.42 (m, 2H), 3.09 (q, 2H), 4.15 (br d, 2H), 7.46 (ddd, 1H), 7.63 (d, |
| | 1H), 7.75 (ddd, 1H), 8.03 (t, 1H), 8.23-8.28 (m, 2H), 8.31 (dd, 1H), 8.68 (d, |
| | 1H), 8.86 (d, 1H). |
| Example 486 | 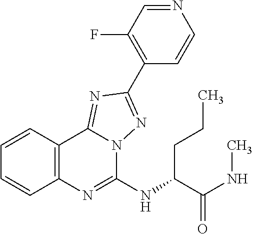 [SMILES: CCC[C@@H](Nc1nc2ccccc2c2nc(-c3ccncc3F)nn12)C(=O)NC] |
| | N²-[2-(3-fluoropyridin-4-yl)[1,2,4]triazolo[1,5-c]quinazolin-5-yl]-N- |
| | methyl-D-norvalinamide |
| | LC-MS (Method 2): Rt = 1.08 min; MS (ESIpos): m/z = 394 [M + H]⁺ |
| | ¹H-NMR (400 MHz, DMSO-d₆): δ [ppm] = 0.91 (t, 3H), 1.32-1.48 (m, 2H), |
| | 1.85-2.01 (m, 2H), 2.63 (d, 3H), 4.74-4.81 (m, 1H), 7.45-7.50 (m, 1H), |
| | 7.65 (d, 1H), 7.73-7.79 (m, 2H), 8.15 (q, 1H), 8.27 (dd, 1H), 8.31 (dd, 1H), |
| | 8.68 (d, 1H), 8.86 (d, 1H). |
| Example 487 | 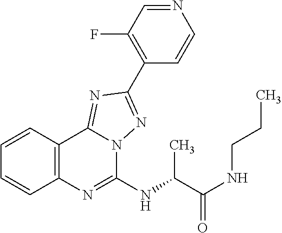 [SMILES: CCCNC(=O)[C@@H](C)Nc1nc2ccccc2c2nc(-c3ccncc3F)nn12] |
| | N²-[2-(3-fluoropyridin-4-yl)[1,2,4]triazolo[1,5-c]quinazolin-5-yl]-N-propyl- |
| | D-alaninamide |
| | LC-MS (Method 20: Rt = 1.06 min; MS (ESIpos): m/z = 394 [M + H]⁺ |
| | ¹H-NMR (400 MHz, DMSO-d₆): δ [ppm] = 0.84 (t, 3H), 1.44 (sxt, 2H), 1.54 (d, |
| | 3H), 3.01-3.15 (m, 2H), 4.77 (quin, 1H), 7.48 (ddd, 1H), 7.65 (d, 1H), 7.74- |
| | 7.83 (m, 2H), 8.19 (t, 1H), 8.26 (dd, 1H), 8.31 (dd, 1H), 8.66-8.69 (m, 1H), |
| | 8.86 (d, 1H). |
| Example 488 | 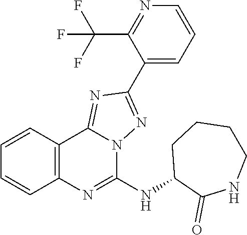 [SMILES: O=C1NCCCC[C@H]1Nc1nc2ccccc2c2nc(-c3cccnc3C(F)(F)F)nn12] |
| | (3R)-3-({2-[2-(trifluoromethyl)pyridin-3-yl][1,2,4]triazolo[1,5- |
| | c]quinazolin-5-yl}amino)azepan-2-one |
| | LC-MS (Method 2): Rt = 1.20 min; MS (ESIpos): m/z = 442 [M + H]⁺ |
| | ¹H-NMR (400 MHz, DMSO-d₆): δ [ppm] = 1.25-1.38 (m, 1H), 1.49-1.60 (m, |
| | 1H), 1.81-1.95 (m, 2H), 1.98-2.07 (m, 1H), 2.31-2.39 (m, 1H), 3.11-3.21 |
| | (m, 1H), 3.33-3.40 (m, 1H and water signal), 4.83 (dd, 1H), 7.48 (ddd, 1H), |
| | 7.69-7.73 (m, 1H), 7.75-7.81 (m, 2H), 7.95 (dd, 1H), 8.21 (dd, 1H), 8.28 |
| | (dd, 1H), 8.48 (dd, 1H), 8.96 (dd, 1H). |
| Example 489 | 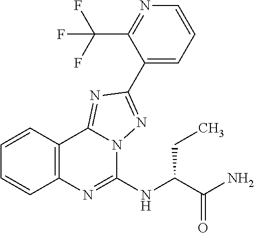 [SMILES: CC[C@@H](Nc1nc2ccccc2c2nc(-c3cccnc3C(F)(F)F)nn12)C(N)=O] |
| | (2R)-2-({2-[2-(trifluoromethyl)pyridin-3-yl][1,2,4]triazolo[1,5- |
| | c]quinazolin-5-yl}amino)butanamide |
| | LC-MS (Method 2): Rt = 1.04 min; MS (ESIpos): m/z = 416 [M + H]⁺ |
| | ¹H-NMR (400 MHz, DMSO-d₆): δ [ppm] = 0.94 (t, 3H), 1.86-1.98 (m, 1H), |
| | 2.01-2.12 (m, 1H), 4.69 (td, 1H), 7.33 (s, 1H), 7.47 (ddd, 1H), 7.62 (d, 1H), |
| | 7.66-7.69 (m, 1H), 7.71 (s, 1H), 7.77 (ddd, 1H), 7.95 (dd, 1H), 8.27 (dd, |
| | 1H), 8.48 (dd, 1H), 8.97 (dd, 1H). |
| Example 490 | 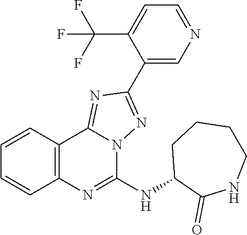 [SMILES: O=C1NCCCC[C@H]1Nc1nc2ccccc2c2nc(-c3cnccc3C(F)(F)F)nn12] |
| | (3R)-3-({2-[4-(trifluoromethyl)pyridin-3-yl][1,2,4]triazolo[1,5- |
| | c]quinazolin-5-yl}amino)azepan-2-one |
| | LC-MS (Method 2): Rt = 1.24 min; MS (ESIpos): m/z = 442 [M + H]⁺ |
| | ¹H-NMR (400 MHz, DMSO-d₆): δ [ppm] = 1.25-1.38 (m, 1H), 1.49-1.61 (m, |
| | 1H), 1.81-.196 (m, 2H), 1.98-2.08 (m, 1H), 2.31-2.39 (m, 1H), 3.11-3.21 |
| | (m, 1H), 3.30-3.41 (m, 1H and water signal), 4.83 (br dd, 1H), 7.49 (ddd, |
| | 1H), 7.69-7.74 (m, 1H), 7.76-7.82 (m, 2H), 8.04 (d, 1H), 8.21 (dd, 1H), |
| | 8.29 (dd, 1H), 9.07 (d, 1H), 9.28 (s, 1H). |
| Example 491 | 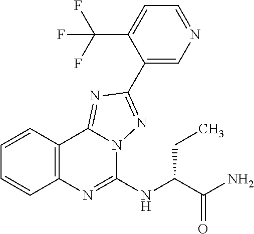 [SMILES: CC[C@@H](Nc1nc2ccccc2c2nc(-c3cnccc3C(F)(F)F)nn12)C(N)=O] |
| | (2R)-2-({2-[4-(trifluoromethyl)pyridin-3-yl][1,2,4]triazolo[1,5- |
| | c]quinazolin-5-yl}amino)butanamide |
| | LC-MS (Method 2): Rt = 1.05 min; MS (ESIpos): m/z = 416 [M + H]⁺ |
| | ¹H-NMR (400 MHz, DMSO-d₆): δ [ppm] = 0.94 (t, 3H), 1.87-1.99 (m, 1H), |
| | 2.01-2.13 (m, 1H), 4.69 (td, 1H), 7.33 (s, 1H), 7.47 (ddd, 1H), 7.64 (d, 1H), |
| | 7.66-7.69 (m, 1H), 7.71 (s, 1H), 7.77 (ddd, 1H), 8.04 (d, 1H), 8.28 (dd, 1H), |
| | 9.07 (d, 1H), 9.2 (s, 1H). |
| Example 492 | 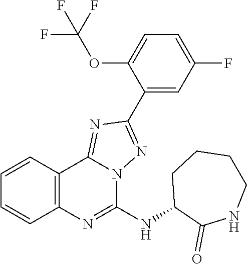 [SMILES: O=C1NCCCC[C@H]1Nc1nc2ccccc2c2nc(-c3cc(F)ccc3OC(F)(F)F)nn12] |
| | (3R)-3-({2-[5-fluoro-2-(trifluoromethoxy)phenyl][1,2,4]triazolo[1,5- |
| | c]quinazolin-5-yl}amino)azepan-2-one |
| | LC-MS (Method 2): Rt = 1.50 min; MS (ESIpos): m/z = 475 [M + H]⁺ |
| | ¹H-NMR (400 MHz, DMSO-d₆): δ [ppm] = 1.25-1.39 (m, 1H), 1.47-1.59 (m, |
| | 1H), 1.81-1.96 (m, 2H), 1.98-2.07 (m, 1H), 2.31-2.39 (m, 1H), 3.12-3.21 |
| | (m, 1H), 4.82 (br dd, 1H), 7.48 (ddd, 1H), 7.59 (ddd, 1H), 7.67-7.73 (m, |
| | 2H), 7.74-7.80 (m, 2H), 8.14 (dd, 1H), 8.19 (dd, 1H), 8.30 (dd, 1H). |
| Example 493 | 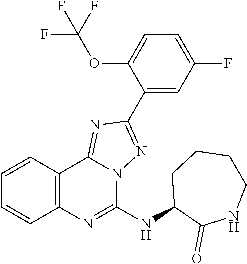 [SMILES: O=C1NCCCC[C@@H]1Nc1nc2ccccc2c2nc(-c3cc(F)ccc3OC(F)(F)F)nn12] |
| | (3S)-3-({2-[5-fluoro-2-(trifluoromethoxy)phenyl][1,2,4]triazolo[1,5- |
| | c]quinazolin-5-yl}amino)azepan-2-one |
| | LC-MS (Method 2): Rt = 1.49 min; MS (ESIpos): m/z = 475 [M + H]⁺ |
| | ¹H-NMR (400 MHz, DMSO-d₆): δ [ppm] = 1.25-1.39 (m, 1H), 1.47-1.59 (m, |
| | 1H), 1.80-1.96 (m, 2H), 1.98-2.07 (m, 1H), 2.31-2.39 (m, 1H), 3.11-3.22 |
| | (m, 1H), 4.82 (br dd, 1H), 7.46-7.51 (m, 1H), 7.58 (ddd, 1H), 7.67-7.72 (m, |
| | 2H), 7.75-7.80 (m, 2H), 8.14 (dd, 1H), 8.18 (br dd, 1H), 8.30 (dd, 1H). |
| Example 494 | 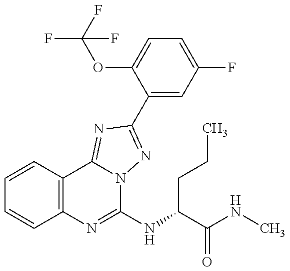 [SMILES: CCC[C@@H](Nc1nc2ccccc2c2nc(-c3cc(F)ccc3OC(F)(F)F)nn12)C(=O)NC] |
| | N²-{2-[5-fluoro-2-(trifluoromethoxy)phenyl][1,2,4]triazolo[1,5- |
| | c]quinazolin-5-yl}-N-methyl-D-norvalinamide |
| | LC-MS (Method 2): Rt = 1.41 min; MS (ESIpos): m/z = 477 [M + H]⁺ |
| | ¹H-NMR (400 MHz, DMSO-d₆): δ [ppm] = 0.91 (t, 3H), 1.31-1.45 (m, 2H), |
| | 1.80-1.99 (m, 2H), 2.64 (d, 3H), 4.77 (td, 1H), 7.48 (ddd, 1H), 7.55 (d, 1H), |
| | 7.59 (ddd, 1H), 7.65 (d, 1H), 7.68-7.73 (m, 1H), 7.73-7.78 (m, 1H), 8.14- |
| | 8.20 (m, 2H), 8.28 (dd, 1H). |
| Example 495 | 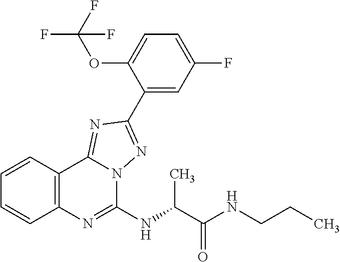 [SMILES: CCCNC(=O)[C@@H](C)Nc1nc2ccccc2c2nc(-c3cc(F)ccc3OC(F)(F)F)nn12] |
| | N²-{2-[5-fluoro-2-(trifluoromethoxy)phenyl][1,2,4]triazolo[1,5- |
| | c]quinazolin-5-yl}-N-propyl-D-alaninamide |
| | LC-MS (Method 2): Rt = 1.47 min; MS (ESIpos): m/z = 477 [M + H]⁺ |
| | ¹H-NMR (400 MHz, DMSO-d₆): δ [ppm] = 0.85 (t, 3H), 1.44 (sxt, 2H), 1.52 (d, |
| | 3H), 3.08 (q, 2H), 2.76 (quin, 1H), 7.46-7.51 (m, 1H), 7.56-7.67 (m, 3H), |
| | 7.68-7.73 (m, 1H), 7.76 (ddd, 1H), 8.15 (dd, 1H), 8.22 (t, 1H), 8.28 (dd, |
| | 1H). |
| Example 496 | 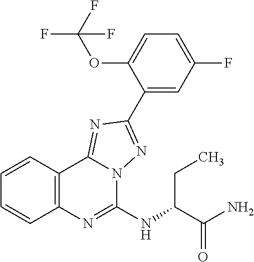 [SMILES: CC[C@@H](Nc1nc2ccccc2c2nc(-c3cc(F)ccc3OC(F)(F)F)nn12)C(N)=O] |
| | (2R)-2-({2-[5-fluoro-2-(trifluoromethoxy)phenyl][1,2,4]triazolo[1,5- |
| | c]quinazolin-5-yl}amino)butanamide |
| | LC-MS (Method 2): Rt = 1.30 min; MS (ESIpos): m/z = 449 [M + H]⁺ |
| | ¹H-NMR (400 MHz, DMSO-d₆): δ [ppm] = 0.94 (t, 3H), 1.86-1.98 (m, 1H), |
| | 2.02-2.13 (m, 1H), 4.69-4.75 (m, 1H), 7.34 (s, 1H), 7.43-7.50 (m, 2H), |
| | 7.59 (ddd, 1H), 7.66 (d, 1H), 7.68-7.73 (m, 1H), 7.73-7.79 (m, 2H), 8.16 |
| | (dd, 1H), 8.29 (dd, 1H). |
| Example 497 | 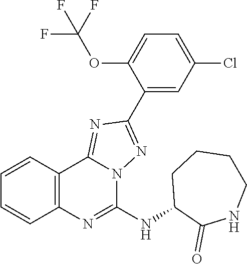 [SMILES: O=C1NCCCC[C@H]1Nc1nc2ccccc2c2nc(-c3cc(Cl)ccc3OC(F)(F)F)nn12] |
| | (3R)-3-({2-[5-chloro-2-(trifluoromethoxy)phenyl][1,2,4]triazolo[1,5- |
| | c]quinazolin-5-yl}amino)azepan-2-one |
| | LC-MS (Method 2): Rt = 1.57 min; MS (ESIpos): m/z = 491 [M + H]⁺ |
| | ¹H-NMR (400 MHz, DMSO-d₆): δ [ppm] = 1.26-1.39 (m, 1H), 1.48-1.59 (m, |
| | 1H), 1.81-1.96 (m, 2H), 1.98-2.07 (m, 1H), 2.31-2.39 (m, 1H), 3.12-3.21 |
| | (m, 1H), 4.82 (br dd, 1H), 7.48 (ddd, 1H), 7.66-7.71 (m, 2H), 7.75-7.82 (m, |
| | 3H), 8.19 (dd, 1H), 8.31 (dd, 1H), 8.39 (d, 1H). |
| Example 498 | 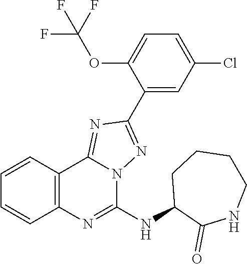 [SMILES: O=C1NCCCC[C@@H]1Nc1nc2ccccc2c2nc(-c3cc(Cl)ccc3OC(F)(F)F)nn12] |
| | (3S)-3-({2-[5-chloro-2-(trifluoromethoxy)phenyl][1,2,4]triazolo[1,5- |
| | c]quinazolin-5-yl}amino)azepan-2-one |
| | LC-MS (Method 2): Rt = 1.60 min; MS (ESIpos): m/z = 491 [M + H]⁺ |
| | ¹H-NMR (400 MHz, DMSO-d₆): δ [ppm] = 1.26-1.38 (m, 1H), 1.48-1.60 (m, |
| | 1H), 1.81-1.96 (m, 2H), 1.98-2.07 (m, 1H), 2.31-2.39 (m, 1H), 3.11-3.22 |
| | (m, 1H), 4.82 (br dd, 1H), 7.48 (ddd, 1H), 7.66-7.72 (m, 2H), 7.74-7.82 (m, |
| | 3H), 8.19 (dd, 1H), 8.31 (dd, 1H), 8.39 (d, 1H). |
| Example 499 | 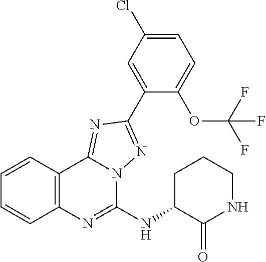 [SMILES: O=C1NCCC[C@H]1Nc1nc2ccccc2c2nc(-c3cc(Cl)ccc3OC(F)(F)F)nn12] |
| | (3R)-3-({2-[5-chloro-2-(trifluoromethoxy)phenyl][1,2,4]triazolo[1,5- |
| | c]quinazolin-5-yl}amino)piperidin-2-one |
| | LC-MS (Method 2): Rt = 1.47 min; MS (ESIpos): m/z = 477 [M + H]⁺ |
| | ¹H-NMR (400 MHz, DMSO-d₆): δ [ppm] = 1.87-1.97 (m, 2H), 1.97-2.09 (m, |
| | 1H), 2.36-2.44 (m, 1H), 3.23-3.29 (m, 2H), 4.67 (dt, 1H), 7.47 (ddd, 1H), |
| | 7.65-7.70 (m, 2H), 7.75 (ddd, 1H), 7.80 (dd, 1H), 7.83 (br s, 1H), 8.08 (d, |
| | 1H), 8.28 (dd, 1H), 8.35 (d, 1H). |
| Example 500 | 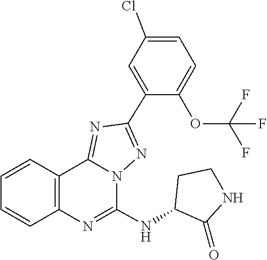 [SMILES: O=C1NCC[C@H]1Nc1nc2ccccc2c2nc(-c3cc(Cl)ccc3OC(F)(F)F)nn12] |
| | (3R)-3-({2-[5-chloro-2-(trifluoromethoxy)phenyl][1,2,4]triazolo[1,5- |
| | c]quinazolin-5-yl}amino)pyrrolidin-2-one |
| | LC-MS (Method 2): Rt = 1.34 min; MS (ESIpos): m/z = 463 [M + H]⁺ |
| | ¹H-NMR (400 MHz, DMSO-d₆): δ [ppm] = 2.23-2.35 (m, 1H), 2.52-2.59 (m, |
| | 1H), 3.35 (br d, 1H), 4.91 (dt, 1H), 7.47 (ddd, 1H), 7.64-7.70 (m, 2H), 7.76 |
| | (ddd, 1H), 7.80 (dd, 1H), 8.03 (s, 1H), 8.20 (d, 1H), 8.27 (dd, 1H), 8.34 (d, |
| | 1H). |
| Example 501 | 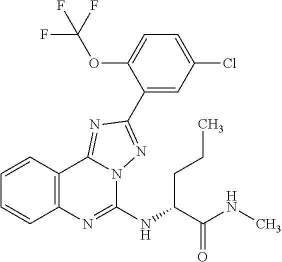 [SMILES: CCC[C@@H](Nc1nc2ccccc2c2nc(-c3cc(Cl)ccc3OC(F)(F)F)nn12)C(=O)NC] |
| | N²-{2-[5-chloro-2-(trifluoromethoxy)phenyl][1,2,4]triazolo[1,5- |
| | c]quinazolin-5-yl}-N-methyl-D-norvalinamide |
| | LC-MS (Method 2): Rt = 1.54 min; MS (ESIpos): m/z = 493 [M + H]⁺ |
| | ¹H-NMR (400 MHz, DMSO-d₆): δ [ppm] = 0.91 (t, 3H), 1.31-1.47 (m, 2H), |
| | 1.81-2.00 (m, 2H), 2.64 (d, 3H), 4.77 (td, 1H), 7.48 (ddd, 1H), 7.61 (d, 1H), |
| | 7.63-7.70 (m, 2H), 7.76 (ddd, 1H), 7.80 (dd, 1H), 8.16 (q, 1H), 8.28 (dd, |
| | 1H), 8.40 (d, 1H). |
| Example 502 | 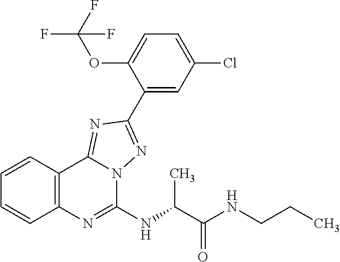 [SMILES: CCCNC(=O)[C@@H](C)Nc1nc2ccccc2c2nc(-c3cc(Cl)ccc3OC(F)(F)F)nn12] |
| | N²-{2-[5-chloro-2-(trifluoromethoxy)phenyl][1,2,4]triazolo[1,5- |
| | c]quinazolin-5-yl}-N-propyl-D-alaninamide |
| | LC-MS (Method 2): Rt = 1.52 min; MS (ESIpos): m/z = 493 [M + H]⁺ |
| | ¹H-NMR (400 MHz, DMSO-d₆): δ [ppm] = 0.85 (t, 3H), 1.44 (sxt, 2H), 1.53 (d, |
| | 3H), 3.04-3.13 (m, 2H), 4.76 (quin, 1H), 7.45-7.50 (m, 1H), 7.63-7.70 (m, |
| | 3H), 7.73-7.82 (m, 2H), 8.21 (t, 1H), 8.29 (dd, 1H), 8.40 (d, 1H). |
| Example 503 | 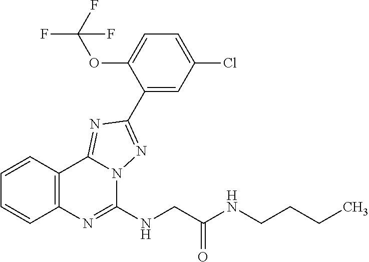 [SMILES: CCCCNC(=O)CNc1nc2ccccc2c2nc(-c3cc(Cl)ccc3OC(F)(F)F)nn12] |
| | N-butyl-N²-{2-[5-chloro-2-(trifluoromethoxy)phenyl][1,2,4]triazolo[1,5- |
| | c]quinazolin-5-yl}glycinamide |
| | LC-MS (Method 2): Rt = 1.48 min; MS (ESIpos): m/z = 493 [M + H]⁺ |
| | ¹H-NMR (400 MHz, DMSO-d₆): δ [ppm] = 0.84 (t, 3H), 1.22-1.33 (m, 2H), |
| | 1.35-1.43 (m, 2H), 3.10 (q, 2H), 4.16 (d, 2H), 7.47 (ddd, 1H), 7.63 (d, 1H), |
| | 7.68 (dd, 1H), 7.75 (ddd, 1H), 7.80 (dd, 1H), 8.05 (t, 1H), 8.09 (t, 1H), 8.28 |
| | (dd, 1H), 8.35 (d, 1H). |
| Example 504 | 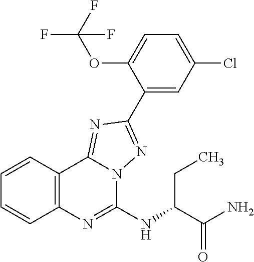 [SMILES: CC[C@@H](Nc1nc2ccccc2c2nc(-c3cc(Cl)ccc3OC(F)(F)F)nn12)C(N)=O] |
| | (2R)-2-({2-[5-chloro-2-(trifluoromethoxy)phenyl][1,2,4]triazolo[1,5- |
| | c]quinazolin-5-yl}amino)butanamide |
| | LC-MS (Method 2): Rt = 1.40 min; MS (ESIpos): m/z = 465 [M + H]⁺ |
| | ¹H-NMR (400 MHz, DMSO-d₆): δ [ppm] = 0.94 (t, 3H), 1.85-1.98 (m, 1H), |
| | 2.01-2.13 (m, 1H), 4.68-4.75 (m, 1H), 7.34 (d, 1H), 7.45-7.52 (m, 2H), |
| | 7.64-7.70 (m, 2H), 7.71-7.82 (m, 3H), 8.29 (dd, 1H), 8.40 (d, 1H). |
| Example 505 | 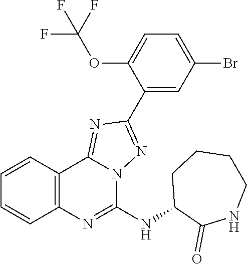 [SMILES: O=C1NCCCC[C@H]1Nc1nc2ccccc2c2nc(-c3cc(Br)ccc3OC(F)(F)F)nn12] |
| | (3R)-3-({2-[5-bromo-2-(trifluoromethoxy)phenyl][1,2,4]triazolo[1,5- |
| | c]quinazolin-5-yl}amino)azepan-2-one |
| | LC-MS (Method 2): Rt = 1.61 min; MS (ESIpos): m/z = 535 [M + H]⁺ |
| | ¹H-NMR (400 MHz, CHLOROFORM-d): δ [ppm] = 1.47-1.56 (m, 1H), 1.66- |
| | 1.78 (m, 1H), 1.93-2.09 (m, 2H), 2.12-2.21 (m, 1H), 2.48 (br d, 1H), 3.31- |
| | 3.40 (m, 1H), 3.47 (ddd, 1H), 4.94 (ddd, 1H), 6.12 (br t, 1H), 7.30-7.35 (m, |
| | 1H), 7.42 (ddd, 1H), 7.63 (dd, 1H), 7.66-7.71 (m, 2H), 7.72-7.75 (m, 1H), |
| | 8.40 (dd, 1H), 8.58 (d, 1H). |
| Example 506 | 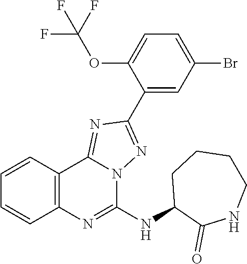 [SMILES: O=C1NCCCC[C@@H]1Nc1nc2ccccc2c2nc(-c3cc(Br)ccc3OC(F)(F)F)nn12] |
| | (3S)-3-({2-[5-bromo-2-(trifluoromethoxy)phenyl][1,2,4]triazolo[1,5- |
| | c]quinazolin-5-yl}amino)azepan-2-one |
| | LC-MS (Method 2): Rt = 1.61 min; MS (ESIpos): m/z = 535 [M + H]⁺ |
| | ¹H-NMR (400 MHz, CHLOROFORM-d): δ [ppm] = 1.47-1.58 (m, 1H), 1.66- |
| | 1.78 (m, 1H), 1.92-2.10 (m, 2H), 2.11-2.20 (m, 1H), 2.44-2.51 (m, 1H), |
| | 3.31-3.40 (m, 1H), 3.47 (ddd, 1H), 4.94 (ddd, 1H), 6.15 (br t, 1H), 7.31- |
| | 7.34 (m, 1H), 7.42 (ddd, 1H), 7.63 (dd, 1H), 7.65-7.71 (m, 2H), 7.71-7.75 |
| | (m, 1H), 8.40 (dd, 1H), 8.58 (d, 1H). |
| Example 507 | 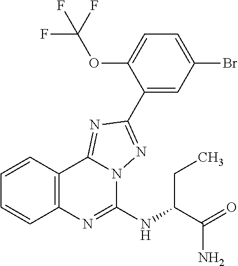 [SMILES: CC[C@@H](Nc1nc2ccccc2c2nc(-c3cc(Br)ccc3OC(F)(F)F)nn12)C(N)=O] |
| | (2R)-2-({2-[5-bromo-2-(trifluoromethoxy)phenyl][1,2,4]triazolo[1,5- |
| | c]quinazolin-5-yl}amino)butanamide |
| | LC-MS (Method 2): Rt = 1.40 min; MS (ESIpos): m/z = 509 [M + H]⁺ |
| | ¹H-NMR (400 MHz, DMSO-d₆): δ [ppm] = 0.94 (t, 3H), 1.86-1.98 (m, 1H), |
| | 2.02-2.13 (m, 1H), 4.71 (td, 1H), 7.33 (s, 1H), 7.44-7.52 (m, 2H), 7.58- |
| | 7.63 (m, 1H), 7.64-7.68 (m, 1H), 7.73 (s, 1H), 7.76 (ddd, 1H), 7.90-7.94 |
| | (m, 1H), 8.29 (dd, 1H), 8.53 (d, 1H). |
| Example 508 | 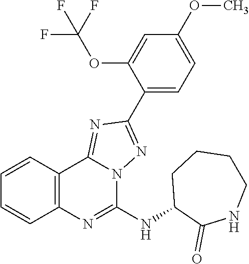 [SMILES: COc1ccc(-c2nc3c4ccccc4nc(N[C@@H]4CCCCNC4=O)n3n2)c(OC(F)(F)F)c1] |
| | (3R)-3-({2-[4-methoxy-2-(trifluoromethoxy)phenyl][1,2,4]triazolo[1,5- |
| | c]quinazolin-5-yl}amino)azepan-2-one |
| | LC-MS (Method 2): Rt = 1.44 min; MS (ESIpos): m/z = 487 [M + H]⁺ |
| | ¹H-NMR (400 MHz, DMSO-d₆): δ [ppm] = 1.24-1.38 (m, 1H), 1.46-1.58 (m, |
| | 1H), 1.81-1.95 (m, 2H), 1.98-2.07 (m, 1H), 2.31-2.39 (m, 1H), 3.11-3.21 |
| | (m, 1H), 391 (s, 3H), 4.81 (br dd, 1H), 7.12 (dd, 1H), 7.24 (dd, 1H), 7.43- |
| | 7.49 (m, 1H), 7.66-7.70 (m, 1H), 7.74 (ddd, 2H), 8.18 (dd, 1H), 8.28 (dd, |
| | 1H), 8.33 (d, 1H). |
| Example 509 | 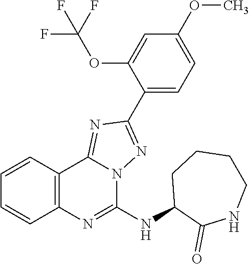 [SMILES: COc1ccc(-c2nc3c4ccccc4nc(N[C@H]4CCCCNC4=O)n3n2)c(OC(F)(F)F)c1] |
| | (3S)-3-({2-[4-methoxy-2-(trifluoromethoxy)phenyl][1,2,4]triazolo[1,5- |
| | c]quinazolin-5-yl}amino)azepan-2-one |
| | LC-MS (Method 2): Rt = 1.43 min; MS (ESIpos): m/z = 487 [M + H]⁺ |
| | ¹H-NMR (400 MHz, DMSO-d₆): δ [ppm] = 1.25-1.39 (m, 1H), 1.46-1.58 (m, |
| | 1H), 1.81-1.96 (m, 2H), 1.98-2.07 (m, 1H), 2.31-2.40 (m, 1H), 3.11-3.21 |
| | (m, 1H), 3.33-3.41 (m, 1H and water signal), 3.91 (s, 3H), 4.81 (br dd, 1H), |
| | 7.12 (d, 1H), 7.24 (dd, 1H), 7.46 (ddd, 1H), 7.68 (d, 1H), 7.72-7.78 (m, 2H), |
| | 8.18 (dd, 1H), 8.28 (dd, 1H), 8.33 (d, 1H). |
| Example 510 | 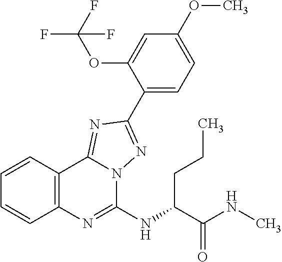 [SMILES: CCC[C@@H](Nc1nc2ccccc2c2nc(-c3ccc(OC)cc3OC(F)(F)F)nn12)C(=O)NC] |
| | N²-{2-[4-methoxy-2-(trifluoromethoxy)phenyl][1,2,4]triazolo[1,5- |
| | c]quinazolin-5-yl}-N-methyl-D-norvalinamide |
| | LC-MS (Method 2): Rt = 1.41 min; MS (ESIpos): m/z = 489 [M + H]⁺ |
| | ¹H-NMR (400 MHz, DMSO-d₆): δ [ppm] = 0.91 (t, 3H), 1.31-1.45 (m, 2H), |
| | 1.79-1.99 (m, 2H), 2.64 (d, 3H), 3.91 (s, 3H), 4.77 (td, 1H), 7.12 (d, 1H), |
| | 7.24 (dd, 1H), 7.42 (d, 1H), 7.44-7.49 (m, 1H), 7.64 (d, 1H), 7.74 (ddd, 1H), |
| | 8.19 (q, 1H), 8.26 (dd, 1H), 8.32 (d, 1H). |
| Example 511 | 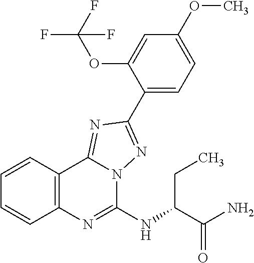 [SMILES: CC[C@@H](Nc1nc2ccccc2c2nc(-c3ccc(OC)cc3OC(F)(F)F)nn12)C(N)=O] |
| | (2R)-2-({2-[4-methoxy-2-(trifluoromethoxy)phenyl][1,2,4]triazolo[1,5- |
| | c]quinazolin-5-yl}amino)butanamide |
| | LC-MS (Method 2): Rt = 1.30 min; MS (ESIpos): m/z = 461 [M + H]⁺ |
| | ¹H-NMR (400 MHz, DMSO-d₆): δ [ppm] = 0.93 (t, 3H), 1.85-1.97 (m, 1H), |
| | 2.01-2.13 (m, 1H), 3.91 (s, 3H), 4.72 (q, 1H), 7.12 (d, 1H), 7.24 (dd, 1H), |
| | 7.32-7.37 (m, 2H), 7.43-7.49 (m, 1H), 7.65 (d, 1H), 7.71-7.78 (m, 2H), |
| | 8.27 (dd, 1H), 8.32 (d, 1H). |
| Example 512 | 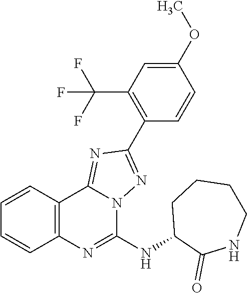 [SMILES: COc1ccc(-c2nc3c4ccccc4nc(N[C@@H]4CCCCNC4=O)n3n2)c(C(F)(F)F)c1] |
| | (3R)-3-({2-[4-methoxy-2-(trifluoromethyl)phenyl][1,2,4]triazolo[1,5- |
| | c]quinazolin-5-yl}amino)azepan-2-one |
| | LC-MS (Method 2): Rt = 1.38 min; MS (ESIpos): m/z = 471 [M + H]⁺ |
| | ¹H-NMR (400 MHz, DMSO-d₆): δ [ppm] = 1.25-1.38 (m, 1H), 1.48-1.60 (m, |
| | 1H), 1.81-1.95 (m, 2H), 1.98-2.07 (m, 1H), 2.31-2.39 (m, 1H), 3.11-3.20 |
| | (m, 1H), 3.34-3.41 (m, 1H and water signal), 3.94 (s, 3H), 4.82 (br dd, 1H), |
| | 7.41-7.49 (m, 3H), 7.69 (d, 1H), 7.71-7.78 (m, 2H), 7.99 (d, 1H), 8.20 (dd, |
| | 1H), 8.26 (dd, 1H). |
| Example 513 | 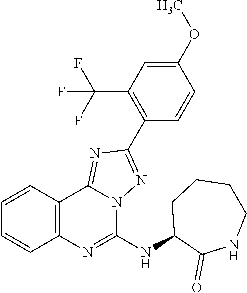 [SMILES: COc1ccc(-c2nc3c4ccccc4nc(N[C@H]4CCCCNC4=O)n3n2)c(C(F)(F)F)c1] |
| | (3S)-3-({2-[4-methoxy-2-(trifluoromethyl)phenyl][1,2,4]triazolo[1,5- |
| | c]quinazolin-5-yl}amino)azepan-2-one |
| | LC-MS (Method 2): Rt = 1.36 min; MS (ESIpos): m/z = 471 [M + H]⁺ |
| | ¹H-NMR (400 MHz, DMSO-d₆): δ [ppm] = 1.25-1.38 (m, 1H), 1.48-1.60 (m, |
| | 1H), 1.81-1.95 (m, 2H), 1.98-2.07 (m, 1H), 2.31-2.38 (m, 1H), 3.11-3.20 |
| | (m, 1H), 3.31-3.41 (m, 1H and water signal), 3.94 (s, 3H), 4.82 (br dd, 1H), |
| | 7.41-7.49 (m, 3H), 7.67-7.71 (m, 1H), 7.71-7.78 (m, 2H), 7.99 (d, 1H), |
| | 8.20 (dd, 1H), 8.26 (dd, 1H). |
| Example 514 | 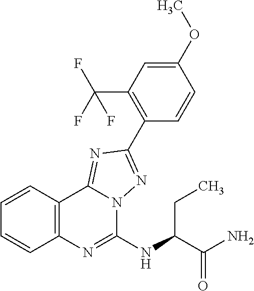 [SMILES: CC[C@H](Nc1nc2ccccc2c2nc(-c3ccc(OC)cc3C(F)(F)F)nn12)C(N)=O] |
| | (2S)-2-({2-[4-methoxy-2-(trifluoromethyl)phenyl][1,2,4]triazolo[1,5- |
| | c]quinazolin-5-yl}amino)butanamide |
| | LC-MS (Method 2): Rt = 1.22 min; MS (ESIpos): m/z = 445 [M + H]⁺ |
| | ¹H-NMR (400 MHz, DMSO-d₆): δ [ppm] = 0.93 (t, 3H), 1.86-1.98 (m, 1H), |
| | 2.00-2.12 (m, 1H), 3.94 (s, 3H), 4.69 (td, 1H), 7.33 (s, 1H), 7.41-7.48 (m, |
| | 3H), 7.51 (d, 1H), 7.63-7.67 (m, 1H), 7.72 (s, 1H), 7.75 (ddd, 1H), 7.98 (d, |
| | 1H), 8.25 (dd, 1H). |
| Example 515 | 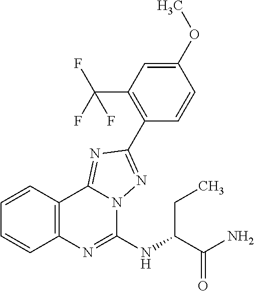 [SMILES: CC[C@@H](Nc1nc2ccccc2c2nc(-c3ccc(OC)cc3C(F)(F)F)nn12)C(N)=O] |
| | (2R)-2-({2-[4-methoxy-2-(trifluoromethyl)phenyl][1,2,4]triazolo[1,5- |
| | c]quinazolin-5-yl}amino)butanamide |
| | LC-MS (Method 2): Rt = 1.23 min; MS (ESIpos): m/z = 445 [M + H]⁺ |
| | ¹H-NMR (400 MHz, DMSO-d₆): δ [ppm] = 0.93 (t, 3H), 1.85-1.98 (m, 1H), |
| | 2.00-2.12 (m, 1H), 3.94 (s, 3H), 4.69 (td, 1H), 7.33 (s, 1H), 7.41-7.48 (m, |
| | 3H), 7.51 (d, 1H), 7.65 (d, 1H), 7.72 (s, 1H), 7.75 (ddd, 1H), 7.98 (d, 1H), |
| | 8.25 (dd, 1H). |
| Example 516 | 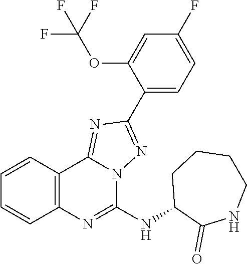 [SMILES: O=C1NCCCC[C@H]1Nc1nc2ccccc2c2nc(-c3ccc(F)cc3OC(F)(F)F)nn12] |
| | (3R)-3-({2-[4-fluoro-2-(trifluoromethoxy)phenyl][1,2,4]triazolo[1,5- |
| | c]quinazolin-5-yl}amino)azepan-2-one |
| | ¹H-NMR (400 MHz, CHLOROFORM-d): δ [ppm] = 1.46-1.59 (m, 1H), 1.66- |
| | 1.78 (m, 1H), 1.93-2.19 (m, 3H), 2.44-2.52 (m, 1H), 3.31-3.40 (m, 1H), |
| | 3.44-3.53 (m, 1H), 4.97-5.05 (m, 1H), 6.11 (br t, 1H), 7.17-7.24 (m, 2H), |
| | 7.42 (ddd, 1H), 7.66-7.75 (m, 2H), 7.78 (d, 1H), 8.37-8.46 (m, 2H). |
| Example 517 | 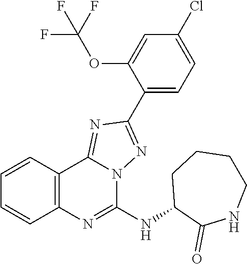 [SMILES: O=C1NCCCC[C@H]1Nc1nc2ccccc2c2nc(-c3ccc(Cl)cc3OC(F)(F)F)nn12] |
| | (3R)-3-({2-[4-chloro-2-(trifluoromethoxy)phenyl][1,2,4]triazolo[1,5- |
| | c]quinazolin-5-yl}amino)azepan-2-one |
| | LC-MS (Method 1): Rt = 1.57 min; MS (ESIpos): m/z = 491 [M + H]⁺ |
| | ¹H-NMR (400 MHz, DMSO-d₆): δ [ppm] = 1.26-1.38 (m, 1H), 1.46-1.59 (m, |
| | 1H), 1.81-1.96 (m, 2H), 1.98-2.07 (m, 1H), 2.32-2.40 (m, 1H), 3.12-3.21 |
| | (m, 1H), 3.31-3.41 (m, 1H and water signal), 4.81 (br dd, 1H), 7.48 (ddd, |
| | 1H), 7.67-7.71 (m, 1H), 7.74-7.81 (m, 4H), 8.20 (dd, 1H), 8.28 (dd, 1H), |
| | 8.42 (d, 1H). |
| Example 518 | 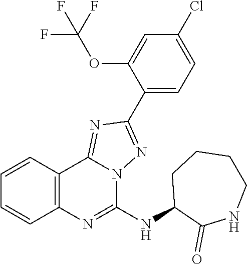 [SMILES: O=C1NCCCC[C@@H]1Nc1nc2ccccc2c2nc(-c3ccc(Cl)cc3OC(F)(F)F)nn12] |
| | (3S)-3-({2-[4-chloro-2-(trifluoromethoxy)phenyl][1,2,4]triazolo[1,5- |
| | c]quinazolin-5-yl}amino)azepan-2-one |
| | LC-MS (Method 1): Rt = 1.57 min; MS (ESIpos): m/z = 491 [M + H]⁺ |
| | ¹H-NMR (400 MHz, DMSO-d₆): δ [ppm] = 1.26-1.39 (m, 1H), 1.47-1.58 (m, |
| | 1H), 1.81-1.95 (m, 2H), 1.98-2.07 (m, 1H), 2.31-2.40 (m, 1H), 3.12-3.21 |
| | (m, 1H), 3.32-3.41 (m, 1H and water signal), 4.81 (br dd, 1H), 7.48 (ddd, |
| | 1H), 7.68-7.72 (m, 1H), 7.74-7.81 (m, 4H), 8.20 (dd, 1H), 8.28 (dd, 1H), |
| | 8.43 (d, 1H). |
| Example 519 | 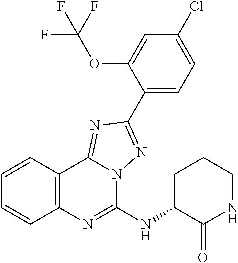 [SMILES: O=C1NCCC[C@H]1Nc1nc2ccccc2c2nc(-c3ccc(Cl)cc3OC(F)(F)F)nn12] |
| | (3R)-3-({2-[4-chloro-2-(trifluoromethoxy)phenyl][1,2,4]triazolo[1,5- |
| | c]quinazolin-5-yl}amino)piperidin-2-one |
| | LC-MS (Method 1): Rt = 1.44 min; MS (ESIpos): m/z = 477 [M + H]⁺ |
| | ¹H-NMR (400 MHz, DMSO-d₆): δ [ppm] = 1.87-2.07 (m, 3H), 2.40-2.47 (m, |
| | 1H), 3.21-3.30 (m, 2H), 4.61-4.69 (m, 1H), 7.46 (ddd, 1H), 7.65-7.68 (m, |
| | 1H), 7.72-7.81 (m, 3H), 7.84 (s, 1H), 8.00 (d, 1H), 8.26 (dd, 1H), 83.4 (d, |
| | 1H). |
| Example 520 | 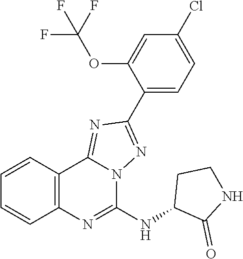 [SMILES: O=C1NCC[C@H]1Nc1nc2ccccc2c2nc(-c3ccc(Cl)cc3OC(F)(F)F)nn12] |
| | (3R)-3-({2-[4-chloro-2-(trifluoromethoxy)phenyl][1,2,4]triazolo[1,5- |
| | c]quinazolin-5-yl}amino)pyrrolidin-2-one |
| | LC-MS (Method 1): Rt = 1.49 min; MS (ESIpos): m/z = 463 [M + H]⁺ |
| | ¹H-NMR (400 MHz, DMSO-d₆): δ [ppm] = 2.22-2.34 (m, 1H), 2.53-2.61 (m, |
| | 1H), 3.35 (br d, 1H), 4.88 (dt, 1H), 7.47 (ddd, 1H), 7.66 (d, 1H), 7.73-7.83 |
| | (m, 3H), 8.02 (s, 1H), 8.11 (d, 1H), 8.26 (dd, 1H), 8.32 (d, 1H). |
| Example 521 | 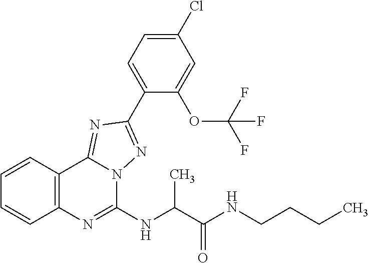 [SMILES: CCCCNC(=O)C(C)Nc1nc2ccccc2c2nc(-c3ccc(Cl)cc3OC(F)(F)F)nn12] |
| | N-butyl-N²-{2-[4-chloro-2-(trifluoromethoxy)phenyl][1,2,4]triazolo[1,5- |
| | c]quinazolin-5-yl}alaninamide |
| | LC-MS (Method 2): Rt = 1.53 min; MS (ESIpos): m/z = 507 [M + H]⁺ |
| | ¹H-NMR (400 MHz, DMSO-d₆): δ [ppm] = 0.85 (t, 3H), 1.22-1.35 (m, 2H), |
| | 1.36-1.45 (m, 2H), 1.52 (d, 3H), 3.07-3.17 (m, 2H), 4.75 (quin, 1H), 7.47 |
| | (ddd, 1H), 7.58 (d, 1H), 7.65 (d, 1H), 7.73-7.81 (m, 3H), 8.20 (t, 1H), 8.27 |
| | (dd, 1H), 8.40 (d, 1H). |
| Example 522 | 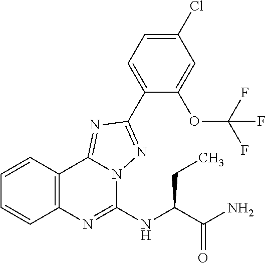 [SMILES: CC[C@H](Nc1nc2ccccc2c2nc(-c3ccc(Cl)cc3OC(F)(F)F)nn12)C(N)=O] |
| | (2S)-2-({2-[4-chloro-2-(trifluoromethoxy)phenyl][1,2,4]triazolo[1,5- |
| | c]quinazolin-5-yl}amino)butanamide |
| | LC-MS (Method 1): Rt = 1.39 min; MS (ESIpos): m/z = 465 [M + H]⁺ |
| | ¹H-NMR (400 MHz, DMSO-d₆): δ [ppm] = 0.93 (t, 3H), 1.85-1.98 (m, 1H), |
| | 2.02-2.14 (m, 1H), 4.72 (td, 1H), 7.35 (s, 1H), 7.42 (d, 1H), 7.48 (ddd, 1H), |
| | 7.65-7.68 (m, 1H), 7.73-7.81 (m, 4H), 8.28 (dd, 1H), 8.42 (d, 1H). |
| Example 523 | 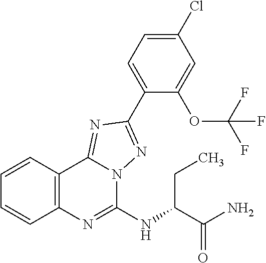 [SMILES: CC[C@@H](Nc1nc2ccccc2c2nc(-c3ccc(Cl)cc3OC(F)(F)F)nn12)C(N)=O] |
| | (2R)-2-({2-[4-chloro-2-(trifluoromethoxy)phenyl][1,2,4]triazolo[1,5- |
| | c]quinazolin-5-yl}amino)butanamide |
| | LC-MS (Method 1): Rt = 1.41 min; MS (ESIpos): m/z = 465 [M + H]⁺ |
| | ¹H-NMR (400 MHz, DMSO-d₆): δ [ppm] 0.93 (t, 3H), 1.86-1.98 (m, 1H), |
| | 2.00-2.13 (m, 1H), 4.72 (td, 1H), 7.35 (s, 1H), 7.42 (d, 1H), 7.47 (ddd, 1H), |
| | 7.66 (d, 1H), 7.73-7.81 (m, 4H), 8.28 (dd, 1H), 8.42 (d, 1H). |
| Example 524 | 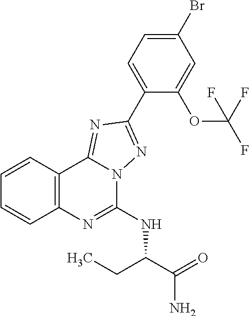 [SMILES: CC[C@H](Nc1nc2ccccc2c2nc(-c3ccc(Br)cc3OC(F)(F)F)nn12)C(N)=O] |
| | (2S)-2-({2-[4-bromo-2-(trifluoromethoxy)phenyl][1,2,4]triazolo[1,5- |
| | c]quinazolin-5-yl}amino)butanamide |
| | LC-MS (Method 2): Rt = 1.44 min; MS (ESIpos): m/z = 509 [M + H]⁺ |
| | ¹H-NMR (400 MHz, DMSO-d₆): δ [ppm] = 0.93 (t, 3H), 1.85-1.97 (m, 1H), |
| | 2.02-2.13 (m, 1H), 4.69-4.75 (m, 1H), 7.35 (s, 1H), 7.41 (d, 1H), 7.48 |
| | (ddd, 1H), 7.66 (d, 1H), 7.73-7.78 (m, 2H), 7.88-7.92 (m, 2H), 8.28 (dd, |
| | 1H), 8.34 (d, 1H). |
| Example 525 | 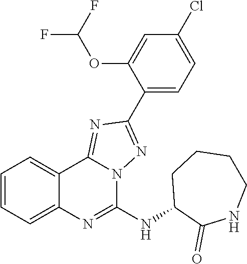 [SMILES: O=C1NCCCC[C@H]1Nc1nc2ccccc2c2nc(-c3ccc(Cl)cc3OC(F)F)nn12] |
| | (3R)-3-({2-[4-chloro-2-(difluoromethoxy)phenyl][1,2,4]triazolo[1,5- |
| | c]quinazolin-5-yl}amino)azepan-2-one |
| | ¹H-NMR (400 MHz, CHLOROFORM-d): δ [ppm] = 1.47-1.60 (m, 1H), 1.66- |
| | 1.77 (m, 1H), 1.93-2.10 (m, 2H), 2.12-2.21 (m, 1H), 2.44-2.51 (m, 1H), |
| | 3.31-3.41 (m, 1H), 3.43-3.52 (m, 1H), 4.91-4.97 (m, 1H), 6.11 (br t, 1H), |
| | 6.86 (t, 1H), 7.38-7.45 (m, 3H), 7.63 (br d, 1H), 7.66-7.75 (m, 2H), 8.30- |
| | 8.34 (m, 1H), 8.36-8.40 (m, 1H). |
| Example 526 | 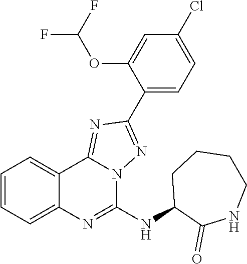 [SMILES: O=C1NCCCC[C@@H]1Nc1nc2ccccc2c2nc(-c3ccc(Cl)cc3OC(F)F)nn12] |
| | (3S)-3-({2-[4-chloro-2-(difluoromethoxy)phenyl][1,2,4]triazolo[1,5- |
| | c]quinazolin-5-yl}amino)azepan-2-one |
| | ¹H-NMR (400 MHz, CHLOROFORM-d): δ [ppm] = 1.47-1.60 (m, 1H), 1.66- |
| | 1.77 (m, 1H), 1.93-2.09 (m, 2H), 2.11-2.20 (m, 1H), 2.44-2.51 (m, 1H), |
| | 3.31-3.40 (m, 1H), 3.47 (ddd, 1H), 4.94 (ddd, 1H), 6.09 (br t, 1H), 6.86 (t, |
| | 1H), 7.39-7.44 (m, 3H), 7.60-7.66 (m, 1H), 7.66-7.71 (m, 1H), 7.71-7.75 |
| | (m, 1H), 8.30-8.33 (m, 1H), 8.38 (dd, 1H). |
| Example 527 | 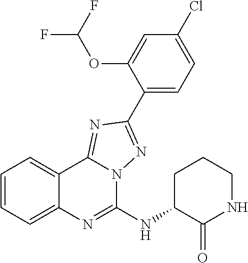 [SMILES: O=C1NCCC[C@H]1Nc1nc2ccccc2c2nc(-c3ccc(Cl)cc3OC(F)F)nn12] |
| | (3R)-3-({2-[4-chloro-2-(difluoromethoxy)phenyl][1,2,4]triazolo[1,5- |
| | c]quinazolin-5-yl}amino)piperidin-2-one |
| | ¹H-NMR (400 MHz, DMSO-d₆): δ [ppm] = 1.86-1.98 (m, 2H), 1.99-2.11 (m, |
| | 1H), 2.32-2.41 (m, 1H), 3.20-3.30 (m, 2H), 4.67 (dt, 1H), 7.39 (t, 1H), 7.45 |
| | (ddd, 1H), 7.55-7.56 (m, 1H), 7.60-7.63 (m, 1H), 7.64-7.68 (m, 1H), 7.75 |
| | (ddd, 1H), 7.82 (br s, 1H), 8.04 (d, 1H), 8.21 (d, 1H), 8.25-8.29 (m, 1H). |
| Example 528 | 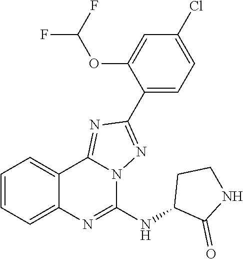 [SMILES: O=C1NCC[C@H]1Nc1nc2ccccc2c2nc(-c3ccc(Cl)cc3OC(F)F)nn12] |
| | (3R)-3-({2-[4-chloro-2-(difluoromethoxy)phenyl][1,2,4]triazolo[1,5- |
| | c]quinazolin-5-yl}amino)pyrrolidin-2-one |
| | LC-MS (Method 2): Rt = 1.26 min; MS (ESIpos): m/z = 445 [M + H]⁺ |
| | ¹H-NMR (400 MHz, CHLOROFORM-d): δ [ppm] = 2.20-2.34 (m, 1H), 3.05- |
| | 3.14 (m, 1H), 3.54-3.60 (m, 2H), 4.87-4.98 (m, 1H), 5.88-5.98 (m, 1H), |
| | 6.59-6.99 (m, 2H), 7.37-7.48 (m, 3H), 7.67-7.73 (m, 1H), 7.73-7.79 (m, |
| | 1H), 8.23-8.30 (m, 1H), 8.38 (br d, 1H). |
| Example 529 | 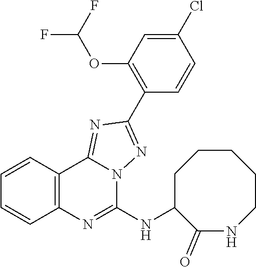 [SMILES: O=C1NCCCCCC1Nc1nc2ccccc2c2nc(-c3ccc(Cl)cc3OC(F)F)nn12] |
| | 3-({2-[4-chloro-2-(difluoromethoxy)phenyl][1,2,4]triazolo[1,5- |
| | c]quinazolin-5-yl}amino)azocan-2-one |
| | LC-MS (Method 2): Rt = 1.46 min; MS (ESIpos): m/z = 487 [M + H]⁺ |
| | ¹H-NMR (400 MHz, DMSO-d₆): δ [ppm] = 1.49-1.76 (m, 7H), 2.25-2.34 (m, |
| | 1H), 3.16-3.25 (m, 1H), 3.59-3.71 (m, 1H), 5.08-5.16 (m, 1H), 7.38 (t, |
| | 1H), 7.47 (ddd, 1H), 7.53-7.57 (m, 2H), 7.61 (dd, 1H), 7.66-7.69 (m, 1H), |
| | 7.76 (ddd, 1H), 7.88 (dd, 1H), 8.25-8.30 (m, 2H). |
| Example 530 | 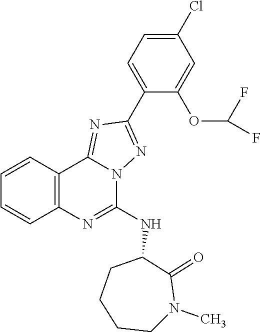 [SMILES: CN1CCCC[C@H](Nc2nc3ccccc3c3nc(-c4ccc(Cl)cc4OC(F)F)nn23)C1=O] |
| | (3S)-3-({2-[4-chloro-2-(difluoromethoxy)phenyl][1,2,4]triazolo[1,5- |
| | c]quinazolin-5-yl}amino)-1-methylazepan-2-one |
| | ¹H-NMR (400 MHz, DMSO-d₆): δ [ppm] = 1.38-1.56 (m, 2H), 1.78-2.02 (m, |
| | 3H), 2.27-2.35 (m, 1H), 3.01 (s, 3H), 2.78 (dd, 1H), 4.97-5.02 (m, 1H), |
| | 7.37 (t, 1H), 7.47 (ddd, 1H), 7.56 (d, 1H), 7.61 (dd, 1H), 7.67-7.71 (m, 1H), |
| | 7.73-7.80 (m, 2H), 8.27-8.30 (m, 2H). |
| Example 531 | 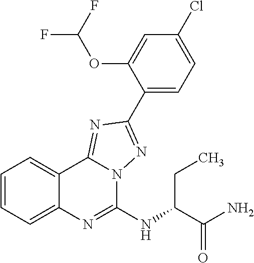 [SMILES: CC[C@@H](Nc1nc2ccccc2c2nc(-c3ccc(Cl)cc3OC(F)F)nn12)C(N)=O] |
| | (2R)-2-({2-[4-chloro-2-(difluoromethoxy)phenyl][1,2,4]triazolo[1,5- |
| | c]quinazolin-5-yl}amino)butanamide |
| | LC-MS (Method 1): Rt = 1.46 min; MS (ESIpos): m/z = 447 [M + H]⁺ |
| | ¹H-NMR (400 MHz, DMSO-d₆): δ [ppm] = 0.95 (t, 3H), 1.86-1.99 (m, 1H), |
| | 2.00-2.12 (m, 1H), 4.71 (td, 1H), 7.33 (s, 1H), 7.38 (t, 1H), 7.42-7.49 (m, |
| | 2H), 7.56 (d, 1H), 7.62 (dd, 1H), 7.65 (d, 1H), 7.72 (s, 1H), 7.75 (ddd, 1H), |
| | 8.25-8.30 (m, 2H). |
| Example 532 | 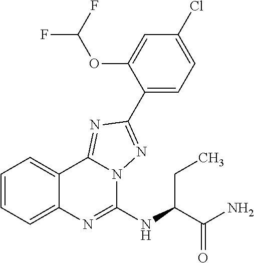 [SMILES: CC[C@H](Nc1nc2ccccc2c2nc(-c3ccc(Cl)cc3OC(F)F)nn12)C(N)=O] |
| | (2S)-2-({2-[4-chloro-2-(difluoromethoxy)phenyl][1,2,4]triazolo[1,5- |
| | c]quinazolin-5-yl}amino)butanamide |
| | LC-MS (Method 1): Rt = 1.32 min; MS (ESIpos): m/z = 447 [M + H]⁺ |
| | ¹H-NMR (400 MHz, DMSO-d₆): δ [ppm] = 0.95 (t, 3H), 1.86-1.98 (m, 1H), |
| | 2.00-2.12 (m, 1H), 4.71 (td, 1H), 7.32 (s, 1H), 7.38 (t, 1H), 7.42-7.49 (m, |
| | 2H), 7.56 (d, 1H), 7.62 (dd, 1H), 7.65 (d, 1H), 7.70-7.78 (m, 2H), 8.25- |
| | 8.30 (m, 2H). |
| Example 533 | 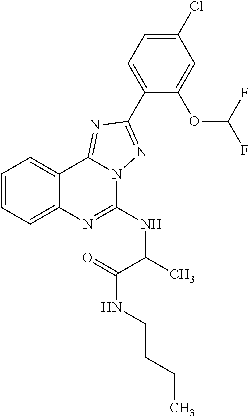 [SMILES: CCCCNC(=O)C(C)Nc1nc2ccccc2c2nc(-c3ccc(Cl)cc3OC(F)F)nn12] |
| | N-butyl-N²-{2-[4-chloro-2-(difluoromethoxy)phenyl][1,2,4]triazolo[1,5- |
| | c]quinazolin-5-yl}alaninamide |
| | LC-MS (Method 2): Rt = 1.42 min; MS (ESIpos): m/z = 489 [M + H]⁺ |
| | ¹H-NMR (400 MHz, DMSO-d₆): δ [ppm] = 0.85 (t, 3H), 1.22-1.34 (m, 2H), |
| | 1.36-1.45 (m, 2H), 1.51 (d, 3H), 3.05-3.18 (m, 2H), 4.75 (quin, 1H), 7.39 |
| | (t, 1H), 7.44-7.49 (m, 1H), 7.56 (d, 1H), 7.60-7.66 (m, 3H), 7.75 (ddd, 1H), |
| | 8.18 (t, 1H), 8.24-8.30 (m, 2H). |
| Example 534 | 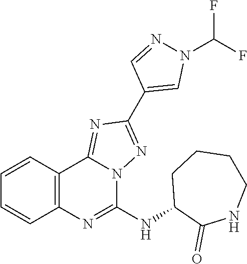 [SMILES: O=C1NCCCC[C@H]1Nc1nc2ccccc2c2nc(-c3cnn(C(F)F)c3)nn12] |
| | (3R)-3-({2-[1-(difluoromethyl)-1H-pyrazol-4-yl][1,2,4]triazolo[1,5- |
| | c]quinazolin-5-yl}amino)azepan-2-one |
| | LC-MS (Method 2): Rₜ = 1.15 min; MS (ESIpos): m/z = 413 [M + H]⁺ |
| | ¹H-NMR (400 MHz, DMSO-d₆): δ [ppm] = 1.26-1.39 (m, 1H), 1.48-1.61 (m, |
| | 1H), 1.81-1.95 (m, 2H), 1.98-2.07 (m, 1H), 2.27-2.35 (m, 1H), 3.11-3.21 |
| | (m, 1H), 3.34-3.42 (m, 1H), 4.83 (dd, 1H), 7.46 (ddd, 1H), 7.63-7.70 (m, |
| | 2H), 7.75 (ddd, 1H), 7.92 (t, 1H), 8.22 (dd, 1H), 8.26 (dd, 1H), 8.44 (s, 1H), |
| | 9.02 (s, 1H). |
| Example 535 | 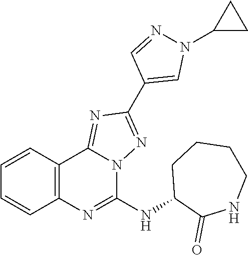 [SMILES: O=C1NCCCC[C@H]1Nc1nc2ccccc2c2nc(-c3cnn(C4CC4)c3)nn12] |
| | (3R)-3-{[2-(1-cyclopropyl-1H-pyrazol-4-yl)[1,2,4]triazolo[1,5- |
| | c]quinazolin-5-yl]amino}azepan-2-one |
| | LC-MS (Method 2): Rt = 1.11 min; MS (ESIpos): m/z = 403 [M + H]⁺ |
| | ¹H-NMR (400 MHz, DMSO-d₆): δ [ppm] = 0.99-1.05 (m, 2H), 1.15-1.20 (m, |
| | 2H), 1.25-1.38 (m, 1H), 1.48-1.60 (m, 1H), 1.81-1.94 (m, 2H), 1.97-2.06 |
| | (m, 1H), 2.26-2.34 (m, 1H), 3.11-3.20 (m, 1H), 3.88 (tt, 1H), 4.82 (br dd, |
| | 1H), 7.44 (ddd, 1H), 7.59 (d, 1H), 7.64-7.67 (m, 1H), 7.73 (ddd, 1H) ,8.07 (d, |
| | 1H), 8.19-8.26 (m, 2H), 8.55 (s, 1H). |
| Example 536 | 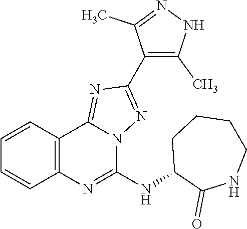 [SMILES: Cc1n[nH]c(C)c1-c1nc2c3ccccc3nc(N[C@@H]3CCCCNC3=O)n2n1] |
| | (3R)-3-{[2-(3,5-dimethyl-1H-pyrazol-4-yl)[1,2,4]triazolo[1,5-c]quinazolin- |
| | 5-yl]amino}azepan-2-one |
| | LC-MS (Method 2): Rₜ = 1.12 min; MS (ESIpos): m/z = 391 [M + H]⁺ |
| | ¹H-NMR (400 MHz, DMSO-d₆): δ [ppm] = 1.25-1.39 (m, 1H), 1.49-1.62 (m, |
| | 1H), 1.80-1.95 (m, 2H), 1.97-2.08 (m, 1H), 2.29-2.38 (m, 1H), 2.60 (br s, |
| | 6H), 3.11-3.21 (m, 1H), 4.78-4.85 (m, 1H), 7.43 (ddd, 1H), 7.63-7.75 (m, |
| | 3H), 8.17 (dd, 1H), 8.26 (dd, 1H), 12.64 (br s, 1H). |
| Example 537 | 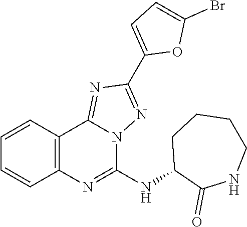 [SMILES: O=C1NCCCC[C@H]1Nc1nc2ccccc2c2nc(-c3ccc(Br)o3)nn12] |
| | (3R)-3-{[2-(5-bromofuran-2-yl)[1,2,4]triazolo[1,5-c]quinazolin-5- |
| | yl]amino}azepan-2-one |
| | LC-MS (Method 2): Rₜ = 1.28 min; MS (ESIneg): m/z = 439 [M − H]⁻ |
| | ¹H-NMR (400 MHz, DSO-d₆): δ [ppm] = 1.22-1.38 (m, 1H), 1.50-1.61 (m, |
| | 1H), 1.81-1.93 (m, 2H), 1.97-2.06 (m, 1H), 2.27-2.34 (m, 1H), 3.12-3.21 |
| | (m, 1H), 4.82 (dd, 1H), 6.89 (d, 1H), 7.36 (d, 1H), 7.45 (ddd, 1H), 7.64-7.72 |
| | (m, 2H), 7.75 (ddd, 1H), 8.21 (dd, 1H), 8.26 (dd, 1H). |
| Example 538 | 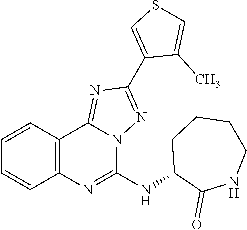 [SMILES: Cc1cscc1-c1nc2c3ccccc3nc(N[C@@H]3CCCCNC3=O)n2n1] |
| | (3R)-3-{[2-(4-methylthiophen-3-yl)[1,2,4]triazolo[1,5-c]quinazolin-5- |
| | yl]amino}azepan-2-one |
| | LC-MS (Method 1): Rt = 1.34 min; MS (ESIpos): m/z = 393 [M + H]⁺ |
| | ¹H-NMR (400 MHz, DMSO-d₆): δ [ppm] = 1.25-1.39 (m, 1H), 1.49-1.61 (m, |
| | 1H), 1.81-1.95 (m, 2H), 1.98-2.07 (m, 1H), 2.30-2.38 (m, 1H), 2.65 (d, |
| | 3H), 3.11-3.21 (m, 1H), 3.31-3.40 (m, 1H and water signal), 4.79-4.85 |
| | (m, 1H), 7.38-7.41 (m, 1H), 7.45 (ddd, 1H), 7.65-7.69 (m, 1H), 7.71-7.76 |
| | (m, 2H), 8.19 (dd, 1H), 8.26-8.30 (m, 1H), 8.32 (d, 1H). |
| Example 539 | 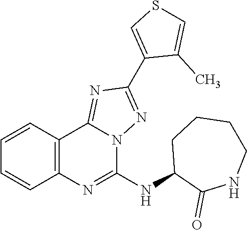 [SMILES: Cc1cscc1-c1nc2c3ccccc3nc(N[C@H]3CCCCNC3=O)n2n1] |
| | (3S)-3-{[2-(4-methylthiophen-3-yl)[1,2,4]triazolo[1,5-c]quinazolin-5- |
| | yl]amino}azepan-2-one |
| | LC-MS (Method 1): Rt = 1.33 min; MS (ESIpos): m/z = 393 [M + H]⁺ |
| | ¹H-NMR (400 MHz, DMSO-d₆): δ [ppm] = 1.25-1.38 (m, 1H), 1.50-1.61 (m, |
| | 1H), 1.80-1.95 (m, 2H), 1.97-2.07 (m, 1H), 2.30-2.38 (m, 1H), 2.65 (d, |
| | 3H), 3.11-3.21 (m, 1H), 3.31-3.41 (m, 1H and water signal), 4.82 (br dd, |
| | 1H), 7.38-7.41 (m, 1H), 7.45 (ddd, 1H), 7.65-7.69 (m, 1H), 7.71-7.76 (m, |
| | 2H), 8.19 (dd, 1H), 8.28 (dd, 1H), 8.32 (d, 1H). |
| Example 540 | 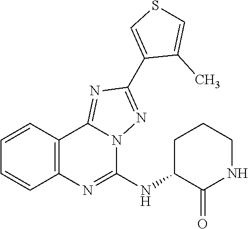 [SMILES: Cc1cscc1-c1nc2c3ccccc3nc(N[C@@H]3CCCNC3=O)n2n1] |
| | (3R)-3-{[2-(4-methylthiophen-3-yl[1,2,4]triazolo[1,5-c]quinazolin-5- |
| | yl]amino}piperidin-2-one |
| | LC-MS (Method 1): Rt = 1.19 min; MS (ESIpos): m/z = 379 [M + H]⁺ |
| | ¹H-NMR (400 MHz, DMSO-d₆): δ [ppm] = 1.85-1.97 (m, 2H), 2.10 (qd, 1H), |
| | 2.27-2.36 (m, 1H), 2.67 (d, 3H), 3.19-3.31 (m, 2H), 4.69 (dt, 1H), 7.37- |
| | 7.40 (m, 1H), 7.43 (ddd, 1H), 76.1-7.65 (m, 1H), 7.72 (ddd, 1H), 7.81 (br s, |
| | 1H), 8.04 (d, 1H), 8.27 (dd, 1H), 8.30 (d, 1H). |
| Example 541 | 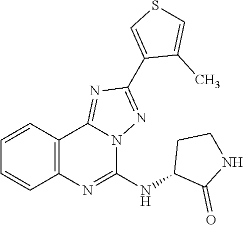 [SMILES: Cc1cscc1-c1nc2c3ccccc3nc(N[C@@H]3CCNC3=O)n2n1] |
| | (3R)-3-{[2-(4-methylthiophen-3-yl[1,2,4]triazolo[1,5-c]quinazolin-5- |
| | yl]amino}pyrrolidin-2-one |
| | LC-MS (Method 1): Rt = 1.12 min; MS (ESIpos): m/z = 365 [M + H]⁺ |
| | ¹H-NMR (400 MHz, DMSO-d₆): δ [ppm] = 2.27-2.39 (m, 1H), 2.68 (d, 3H), |
| | 3.34-3.37 (m, 1H), 4.94 (dt, 1H), 7.37-7.39 (m, 1H), 7.43 (ddd, 1H), 7.60- |
| | 7.64 (m, 1H), 7.73 (ddd, 1H), 8.01 (s, 1H), 8.14 (d, 1H), 8.27 (dd, 1H), 8.30 |
| | (d, 1H). |
| Example 542 | 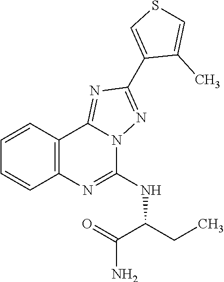 [SMILES: CC[C@@H](Nc1nc2ccccc2c2nc(-c3cscc3C)nn12)C(N)=O] |
| | (2R)-2-{[2-(4-methylthiophen-3-yl[1,2,4]triazolo[1,5-c]quinazolin-5- |
| | yl]amino}butanamide |
| | LC-MS (Method 1): Rt = 1.19 min; MS (ESIpos): m/z = 367 [M + H]⁺ |
| | ¹H-NMR (400 MHz, DMSO-d₆): δ [ppm] = 0.95 (t, 3H), 1.87-1.99 (m, 1H), |
| | 2.01-2.14 (m, 1H), 2.66 (d, 3H), 4.71 (td, 1H), 7.33 (s, 1H), 7.38-7.40 (m, |
| | 1H), 7.42-7.47 (m, 2H), 7.62-7.65 (m, 1H), 7.70-7.76 (m, 2H), 8.28 (dd, |
| | 1H), 8.32 (d, 1H). |
| Example 543 | 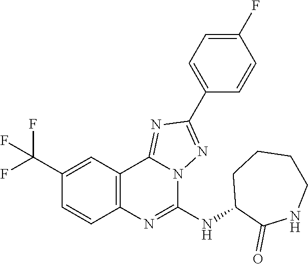 [SMILES: O=C1NCCCC[C@H]1Nc1nc2ccc(C(F)(F)F)cc2c2nc(-c3ccc(F)cc3)nn12] |
| | (3R)-3-{[2-(4-fluorophenyl)-9-(trifluoromethyl)[1,2,4]triazolo[1,5- |
| | c]quinazolin-5-yl]amino}azepan-2-one |
| | LC-MS (Method 2): Rₜ = 1.52 min; MS (ESIpos): m/z = 459 [M + H]⁺ |
| | ¹H-NMR (400 MHz, DMSO-d₆): δ [ppm] = 1.25-1.41 (m, 1H), 1.52-1.68 (m, |
| | 1H), 1.79-1.97 (m, 2H), 1.99-2.09 (m, 1H), 2.27-2.38 (m, 1H), 3.10-3.22 |
| | (m, 1H), 3.35-3.44 (m, 1H), 4.86 (br dd, 1H), 7.37-7.49 (m, 2H), 7.81 (d, |
| | 1H), 7.95-8.05 (m, 2H), 8.26 (dd, 1H), 8.29-8.37 (m, 2H), 8.51 (d, 1H). |
| Example 544 | 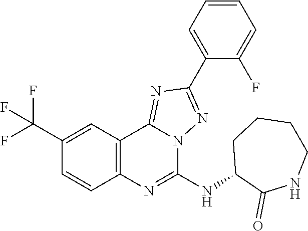 [SMILES: O=C1NCCCC[C@H]1Nc1nc2ccc(C(F)(F)F)cc2c2nc(-c3ccccc3F)nn12] |
| | (3R)-3-{[2-(2-fluorophenyl)-9-(trifluoromethyl)[1,2,4]triazolo[1,5- |
| | c]quinazolin-5-yl]amino}azepan-2-one |
| | LC-MS (Method 2): Rₜ = 1.47 min; MS (ESIpos): m/z = 459 [M + H]⁺ |
| | ¹H-NMR (400 MHz, DMSO-d₆): δ [ppm]= 1.26-1.39 (m, 1H), 1.50-1.68 (m, |
| | 1H), 1.80-1.96 (m, 2H), 1.99-2.09 (m, 1H), 2.31-2.41 (m, 1H), 3.10-3.23 |
| | (m, 1H), 3.35-3.44 (m, 1H), 4.87 (br dd, 1H), 7.40-7.53 (m, 2H), 7.59-7.70 |
| | (m, 1H), 7.84 (d, 1H), 7.95-8.07 (m, 2H), 8.22-8.36 (m, 2H), 8.53 (d, 1H). |
| Example 545 | 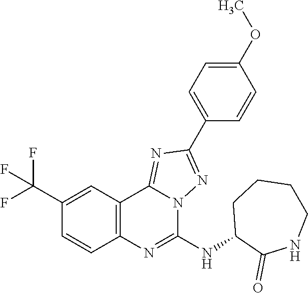 [SMILES: COc1ccc(-c2nc3c4cc(C(F)(F)F)ccc4nc(N[C@@H]4CCCCNC4=O)n3n2)cc1] |
| | (3R)-3-{[2-(4-methoxyphenyl)-9-(trifluoromethyl)[1,2,4]triazolo[1 ,5- |
| | c]quinazolin-5-yl]amino}azepan-2-one |
| | LC-MS (Method 2): Rₜ = 1.48 min; MS (ESIpos): m/z = 471 [M + H]⁺ |
| | ¹H-NMR (400 MHz, DMSO-d₆): δ [[ppm] = 1.22-1.45 (m, 1H), 1.49-1.69 (m, |
| | 1H), 1.78-1.95 (m, 2H), 1.96-2.13 (m, 1H), 2.22-2.33 (m, 1H), 3.09-3.22 |
| | (m, 1H), 3.36-3.44 (m, 1H), 3.86 (s, 3H), 4.87 (br dd, 1H), 7.12-7.17 (m, |
| | 2H), 7.80 (d, 1H), 7.95-8.01 (m, 2H), 8.20-8.27 (m, 3H), 8.51 (d, 1H). |
| Example 546 | 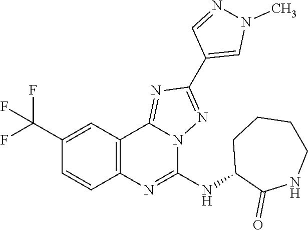 [SMILES: Cn1cc(-c2nc3c4cc(C(F)(F)F)ccc4nc(N[C@@H]4CCCCNC4=O)n3n2)cn1] |
| | (3R)-3-{[2-(1-methyl-1H-pyrazol-4-yl)-9- |
| | (trifluoromethyl)[1,2,4]triazolo[1,5-c]quinazolin-5-yl]amino}azepan-2- |
| | one |
| | LC-MS (Method 2): Rₜ = 1.22 min; MS (ESIpos): m/z = 445 [M + H]⁺ |
| | ¹H-NMR (400 MHz, DMSO-d₆): δ [ppm] = 1.26-1.38 (m, 1H), 1.51-1.62 (m, |
| | 1H), 1.81-1.94 (m, 2H), 1.97-2.07 (m, 1H), 2.27-2.35 (m, 2H), 3.11-3.21 |
| | (m, 1H), 3.96 (s, 3H), 4.86 (br d, 1H), 7.81 (d, 1H), 7.84-7.90 (m, 1H), 7.99 |
| | (dd, 1H), 8.09 (d, 1H) ,8.25 (dd, 1H), 8.47 (d, 1H), 8.52 (s, 1H). |
| Example 547 | 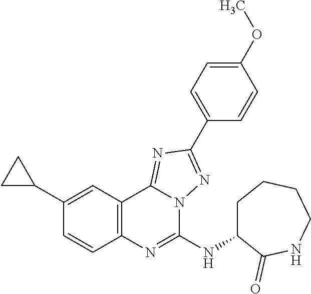 [SMILES: COc1ccc(-c2nc3c4cc(C5CC5)ccc4nc(N[C@@H]4CCCCNC4=O)n3n2)cc1] |
| | (3R)-3-{[9-cyclopropyl-2-(4-methoxyphenyl)[1,2,4]triazolo[1,5- |
| | c]quinazolin-5-yl]amino}azepan-2-one |
| | LC-MS (Method 2): Rₜ = 1.44 min; MS (ESIpos): m/z = 443 [M + H]⁺ |
| | ¹H-NMR (400 MHz, DMSO-d₆): δ [ppm] = 0.77-0.82 (m, 2H), 1.02-1.08 (m, |
| | 2H), 1.25-1.38 (m, 1H), 1.49-1.61 (m, 1H), 1.80-1.94 (m, 2H), 1.97-2.06 |
| | (m, 1H), 2.12-2.20 (m, 1H), 2.27-2.34 (m, 1H), 3.11-3.20 (m, 1H), 3.34- |
| | 3.40 (m, 1H), 3.86 (s, 3H), 4.81 (dd, 1H), 7.12-7.17 (m, 2H), 7.45 (dd, 1H), |
| | 7.56 (d, 1H), 7.63 (d, 1H), 7.96 (d, 1H), 8.18-8.25 (m, 3H). |
| Example 548 | 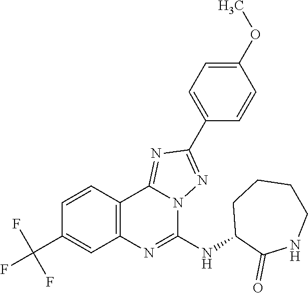 [SMILES: COc1ccc(-c2nc3c4ccc(C(F)(F)F)cc4nc(N[C@@H]4CCCCNC4=O)n3n2)cc1] |
| | (3R)-3-{[2-(4-methoxyphenyl)-8-(trifluoromethyl)[1,2,4]triazolo[1,5- |
| | c]quinazolin-5-yl]amino}azepan-2-one |
| | LC-MS (Method 2): Rₜ = 1.48 min; MS (ESIpos): m/z = 471 [M + H]⁺ |
| | ¹H-NMR (400 MHz, DMSO-d₆): δ [ppm] = 1.25-1.40 (m, 1H), 1.52-1.64 (m, |
| | 1H), 1.82-1.97 (m, 2H), 1.98-2.08 (m, 1H), 2.28-2.36 (m, 1H), 3.13-3.23 |
| | (m, 1H), 3.34-3.41 (m, 1H), 3.86 (s, 3H), 4.85 (br dd, 1H), 7.13-7.16 (m, |
| | 2H), 7.71 (dd, 1H), 7.89-7.94 (m, 2H), 8.20-8.27 (m, 3H), 8.48 (d, 1H). |
| Example 549 | 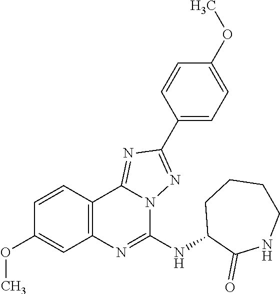 [SMILES: COc1ccc(-c2nc3c4ccc(OC)cc4nc(N[C@@H]4CCCCNC4=O)n3n2)cc1] |
| | (3R)-3-{[8-methoxy-2-(4-methoxyphenyl)[1,2,4]triazolo[1,5-c]quinazolin- |
| | 5-yl]amino}azepan-2-one |
| | LC-MS (Method 2): Rₜ = 1.29 min; MS (ESIpos): m/z = 433 [M + H]⁺ |
| | ¹H-NMR (400 MHz, DMSO-d₆): δ [ppm] = 1.24-1.40 (m, 1H), 1.50-1.64 (m, |
| | 1H), 1.81-1.96 (m, 2H), 1.97-2.07 (m, 1H), 2.25-2.35 (m, 1H), 3.10-3.21 |
| | (m, 1H), 3.30-3.34 (m, 1H), 3.85 (s, 3H), 3.92 (s, 3H), 4.82 (dd, 1H), 7.04 |
| | (dd, 1H), 7.09 (d, 1H), 7.10-7.15 (m, 2H), 7.68 (d, 1H), 8.15-8.23 (m, 4H). |
| Example 550 | 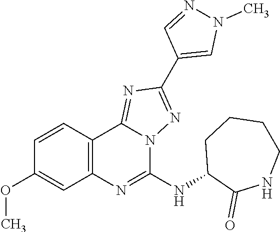 [SMILES: COc1ccc2c(c1)nc(N[C@@H]1CCCCNC1=O)n1nc(-c3cnn(C)c3)nc21] |
| | (3R)-3-{[8-methoxy-2-(1-methyl-1H-pyrazol-4-yl[1,2,4]triazolo[1,5- |
| | c]quinazolin-5-yl]amino}azepan-2-one |
| | LC-MS (Method 2): Rₜ = 1.00 min; MS (ESIpos): m/z = 407 [M + H]⁺ |
| | ¹H-NMR (400 MHz, DMSO-d₆): δ [ppm] = 1.23-1.40 (m, 1H), 1.47-1.62 (m, |
| | 1H), 1.80-1.96 (m, 2H), 1.98-2.06 (m, 1H), 2.29 (br d, 1H), 3.09-3.22 (m, |
| | 1H), 3.91 (s, 3H), 3.94 (s, 3H), 4.81 (dd, 1H), 7.03 (dd, 1H), 7.08 (d, 1H), 7.58 |
| | (d, 1H), 8.04 (s, 1H), 8.12 (d, 1H), 8.21 (dd, 1H), 8.46 (s, 1H). |
| Example 551 | 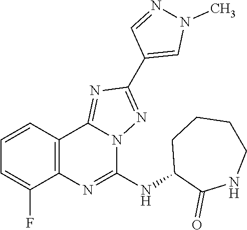 [SMILES: Cn1cc(-c2nc3c4cccc(F)c4nc(N[C@@H]4CCCCNC4=O)n3n2)cn1] |
| | (3R)-3-{[7-fluoro-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.04 min; MS (ESIpos): m/z = 395 [M + H]⁺ |
| | ¹H-NMR (400 MHz, DMSO-d₆): δ [ppm] = 1.25-1.39 (m, 1H), 1.46-1.59 (m, |
| | 1H), 1.80-1.90 (m, 2H), 1.98-2.07 (m, 1H), 2.30-2.37 (m, 1H), 3.11-3.21 |
| | (m, 1H), 3.95 (s, 3H), 4.82 (br dd, 1H), 7.40 (td, 1H), 7.59 (ddd, 1H), 7.74 (d, |
| | 1H), 8.03-8.07 (m, 1H), 8.07 (d, 1H), 8.23 (dd, 1H), 8.51 (s, 1H). |
| Example 552 | 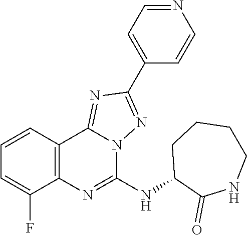 [SMILES: O=C1NCCCC[C@H]1Nc1nc2c(F)cccc2c2nc(-c3ccncc3)nn12] |
| | (3R)-3-{[7-fluoro-2-(pyridin-4-yl)[1,2,4]triazolo[1,5-c]quinazolin-5- |
| | yl]amino}azepan-2-one |
| | LC-MS (Method 2): Rₜ = 1.15 min; MS (ESIpos): m/z = 392 [M + H]⁺ |
| | ¹H-NMR (400 MHz, DMSO-d₆): δ [ppm] = 1.37-1.45 (m, 1H), 1.50-1.65 (m, |
| | 1H), 1.78-1.91 (m, 2H), 2.00-2.07 (m, 1H), 2.30-2.40 (m, 1H), 3.12-3.22 |
| | (m, 1H), 4.81-4.88 (m, 1H), 7.45 (td, 1H), 7.64 (ddd, 1H), 7.94 (d, 1H), 8.14 |
| | (d, 1H), 8.18-8.22 (m, 2H), 8.23-8.28 (m, 1H), 8.82-8.85 (m, 2H). |
| Example 553 | 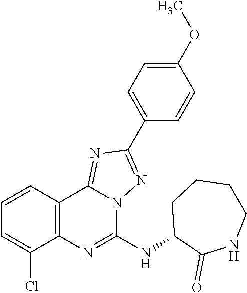 [SMILES: COc1ccc(-c2nc3c4cccc(Cl)c4nc(N[C@@H]4CCCCNC4=O)n3n2)cc1] |
| | (3R)-3-{[7-chloro-2-(4-methoxyphenyl)[1,2,4]triazolo[1,5-c]quinazolin-5- |
| | yl]amino}azepan-2-one |
| | LC-MS (Method 2): Rₜ = 1.45 min; MS (ESIpos): m/z = 437 [M + H]⁺ |
| | ¹H-NMR (400 MHz, DMSO-d₆): δ [ppm] = 1.27-1.40 (m, 1H), 1.49-1.62 (m, |
| | 1H), 1.82-1.96 (m, 2H), 2.01-2.10 (m, 1H), 2.42 (br d, 1H), 3.14-3.23 (m, |
| | 1H), 3.28-3.38 (m, 1H and water signal), 3.86 (s, 3H), 4.84 (br dd, 1H), 7.12- |
| | 7.17 (m, 2H), 7.41 (t, 1H), 7.81 (d, 1H), 7.90 (dd, 1H), 8.19-8.27 (m, 4H). |
| Example 554 | 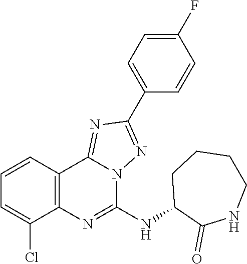 [SMILES: O=C1NCCCC[C@H]1Nc1nc2c(Cl)cccc2c2nc(-c3ccc(F)cc3)nn12] |
| | (3R)-3-{[7-chloro-2-(4-fluorophenyl)[1,2,4]triazolo[1,5-c]quinazolin-5- |
| | yl]amino}azepan-2-one |
| | LC-MS (Method 2): Rₜ = 1.49 min; MS (ESIpos): m/z = 425 [M + H]⁺ |
| | ¹H-NMR (400 MHz, DMSO-d₆): δ [ppm] = 1.27-1.40 (m, 1H), 1.49-1.62 (m, |
| | 1H), 1.81-1.96 (m, 2H), 2.01-2.10 (m, 1H), 2.42 (br d, 1H), 3.13-3.24 (m, |
| | 1H), 4.84 (dd, 1H), 7.39-7.48 (m, 3H), 7.85 (d, 1H), 7.92 (dd, 1H), 8.22-8.29 |
| | (m, 2H), 8.30-8.36 (m, 2H). |
| Example 555 | 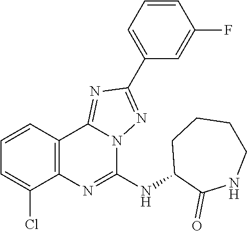 [SMILES: O=C1NCCCC[C@H]1Nc1nc2c(Cl)cccc2c2nc(-c3cccc(F)c3)nn12] |
| | (3R)-3-{[7-chloro-2-(3-fluorophenyl)[1,2,4]triazolo[1,5-c]quianzolin-5- |
| | yl]amino}azepan-2-one |
| | ¹H-NMR (400 MHz, DMSO-d₆): δ [ppm] = 1.27-1.41 (m, 1H), 1.50-1.63 (m, |
| | 1H), 1.83-1.96 (m, 2H), 2.02-2.10 (m, 1H), 2.42 (br d, 1H), 3.14-3.24 (m, |
| | 1H), 3.28-3.38 (m, 1H and water signal), 4.85 (dd, 1H), 7.40-7.47 (m, 2H), |
| | 7.66 (td, 1H), 7.88 (d, 1H), 792 (dd, 1H), 7.99 (ddd, 1H), 8.13 (dt, 1H), 8.22- |
| | 8.29 (m, 2H). |
| Example 556 | 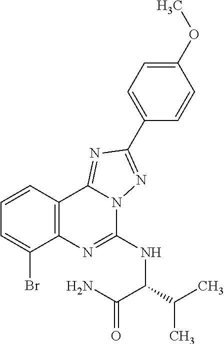 [SMILES: COc1ccc(-c2nc3c4cccc(Br)c4nc(N[C@@H](C(N)=O)C(C)C)n3n2)cc1] |
| | N²-[7-bromo-2-(4-methoxyphenyl)[1,2,4]triazolo[1,5-c]quinazolin-5-yl]- |
| | D-valinamide |
| | LC-MS (Method 2): Rt = 1.34 min; MS (ESIpos): m/z = 469 [M + H]⁺ |
| | ¹H-NMR (400 MHz, DMSO-d₆): δ [ppm] = 1.00 (d, 3H), 1.09 (d, 3H), 2.33- |
| | 2.45 (m, 1H), 3.86 (s, 3H), 4.78 (dd, 1H), 7.11-7.17 (m, 2H), 7.34 (t, 1H), |
| | 7.43-7.48 (m, 2H), 7.83 (s, 1H), 8.06 (dd, 1H), 8.22-8.27 (m, 2H), 8.30 |
| | (dd, 1H). |
| Example 557 | 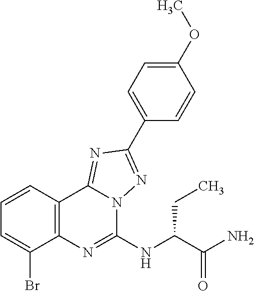 [SMILES: CC[C@@H](Nc1nc2c(Br)cccc2c2nc(-c3ccc(OC)cc3)nn12)C(N)=O] |
| | (2R)-2-{[7-bromo-2-(4-methoxyphenyl)[1,2,4]triazolo[1,5-c]quinazolin-5- |
| | yl]amino}butanamide |
| | LC-MS (Method 2): Rt = 1.29 mi; MS (ESIpos): m/z = 457 [M + H]⁺ |
| | ¹H-NMR (400 MHz, DMSO-d₆): δ [ppm] = 0.97 (t, 3H), 1.90-2.03 (m, 1H), |
| | 2.14-2.27 (m, 1H), 3.86 (s, 3H), 4.74 (td, 1H), 7.12-7.18 (m, 2H), 7.33 (t, |
| | 1H), 7.36 (s, 1H), 7.75 (s, 1H), 7.82 (d, 1H), 8.06 (dd, 1H), 8.22-8.27 (m, |
| | 2H), 8.30 (dd, 1H). |
| Example 558 | 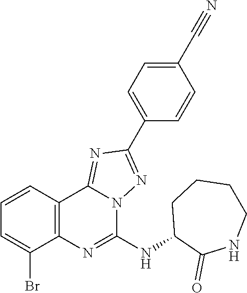 [SMILES: N#Cc1ccc(-c2nc3c4cccc(Br)c4nc(N[C@@H]4CCCCNC4=O)n3n2)cc1] |
| | 4-(7-bromo-5-{[(3R)-2-oxoazepan-3-yl]amino}[1,2,4]triazolo[1,5- |
| | c]quinazolin-2-yl)benzonitrile |
| | LC-MS (Method 2): Rt = 1.46 min; MS (ESIpos): m/z = 476 [M + H]⁺ |
| | ¹H-NMR (400 MHz, DMSO-d₆): δ [ppm] = 1.27-1.41 (m, 1H), 1.50-1.62 (m, |
| | 1H), 1.82-2.00 (m, 2H), 2.02-2.11 (m, 1H), 2.44 (br d, 1H), 3.15-3.25 (m, |
| | 1H), 3.27-3.33 (m, 1H and water signal), 4.83 (br dd, 1H), 7.36 (t, 1H), 7.88 |
| | (d, 1H), 8.04-8.08 (m, 2H), 8.10 (dd, 1H), 8.26 (dd, 1H), 83.1 (dd, 1H), 8.41- |
| | 8.45 (m, 2H). |
| Example 559 | 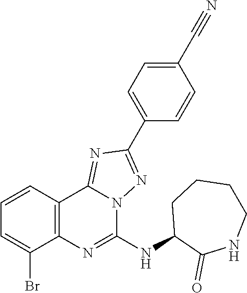 [SMILES: N#Cc1ccc(-c2nc3c4cccc(Br)c4nc(N[C@H]4CCCCNC4=O)n3n2)cc1] |
| | 4-(7-bromo-5-{[(3S)-2-oxoazepan-3-yl]amino}[1,2,4]triazolo[1,5- |
| | c]quinazolin-2-yl)benzonitrile |
| | LC-MS (Method 2): Rt = 1.47 min; MS (ESIpos): m/z = 476 [M + H]⁺ |
| | ¹H-NMR (400 MHz, DMSO-d₆): δ [ppm] = 1.28-1.41 (m, 1H), 1.50-1.62 (m, |
| | 1H), 1.83-2.00 (m, 2H), 2.02-2.11 (m, 1H), 2.40-247 (m, 1H), 3.15-3.25 |
| | (m, 1H), 3.27-3.34 (m, 1H and water signal), 4.83 (br dd, 1H), 7.36 (t, 1H), |
| | 7.88 (d, 1H), 8.04-8.08 (m, 2H), 8.09 (dd, 1H), 8.26 (dd, 1H), 8.31 (dd, 1H), |
| | 8.40-8.45 (m, 2H). |
| Example 560 | 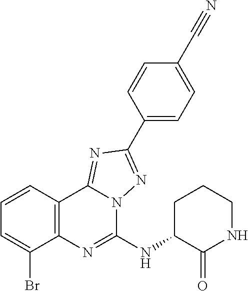 [SMILES: N#Cc1ccc(-c2nc3c4cccc(Br)c4nc(N[C@@H]4CCCNC4=O)n3n2)cc1] |
| | 4-(7-bromo-5-{[(3R)-2-oxopiperidin-3-yl]amino}[1,2,4]triazolo[1,5- |
| | c]quinazolin-2-yl)benzonitrile |
| | LC-MS (Method 2): Rt = 1.28 min; MS (ESIpos): m/z = 462 [M + H]⁺ |
| | ¹H-NMR (400 MHz, DMSO-d₆): δ [ppm] = 1.80-2.02 (m, 2H), 2.25-2.38 (m, |
| | 2H), 3.20-3.43 (m, 2H and water signal), 4.50-4.59 (m, 1H), 7.34 (t, 1H), |
| | 7.83 (br s, 1H), 8.05-8.11 (m, 3H), 8.29 (dd, 1H), 8.43 (d, 2H), 8.65 (d, 1H). |
| Example 561 | 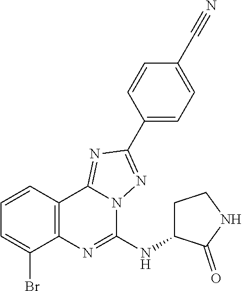 [SMILES: N#Cc1ccc(-c2nc3c4cccc(Br)c4nc(N[C@@H]4CCNC4=O)n3n2)cc1] |
| | 4-(7-bromo-5-{[(3R)-2-oxopyrrolidin-3-yl]amino}[1,2,4]triazolo[1,5- |
| | c]quinazolin-2-yl)benzonitrile |
| | LC-MS (Method 2): Rt = 1.19 min; MS (ESIpos): m/z = 448 [M + H]⁺ |
| | ¹H-NMR (400 MHz, DMSO-d₆): δ [ppm] = 2.40-2.58 (m, 2H and DMSO-D₆ |
| | signal), 3.36-3.47 (m, 2H and water signal), 4.80-4.89 (m, 1H), 7.35 (t, |
| | 1H), 8.03-8.12 (m, 4H), 8.31 (dd, 1H), 8.44 (d, 2H), 8.67 (d, 1H). |
| Example 562 | 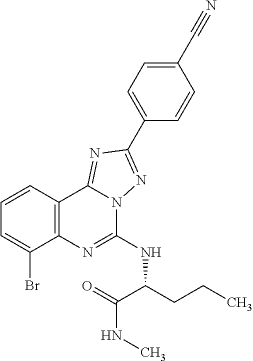 [SMILES: CCC[C@@H](Nc1nc2c(Br)cccc2c2nc(-c3ccc(C#N)cc3)nn12)C(=O)NC] |
| | N²-[7-bromo-2-(4-cyanophenyl)[1,2,4]triazolo[1,5-c]quinazolin-5-yl]-N- |
| | methyl-D-norvalinamide |
| | LC-MS (Method 2): Rt = 1.38 min; MS (ESIpos): m/z = 478 [M + H]⁺ |
| | ¹H-NMR (400 MHz, DMSO-d₆): δ [ppm] = 0.94 (t, 3H), 1.34-1.52 (m, 2H), |
| | 1.88-2.07 (m, 2H), 2.64 (d, 3H), 4.78-4.85 (m, 1H), 7.35 (t, 1H), 8.05- |
| | 8.15 (m, 4H), 8.20 (br d, 1H), 8.31 (dd, 1H), 8.47 (d, 2H). |
| Example 563 | 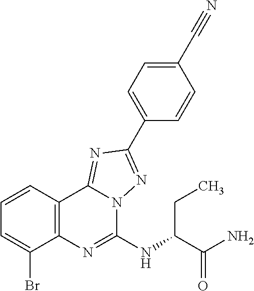 [SMILES: CC[C@@H](Nc1nc2c(Br)cccc2c2nc(-c3ccc(C#N)cc3)nn12)C(N)=O] |
| | (2R)-2-{[7-bromo-2-(4-cyanophenyl)[1,2,4]triazolo[1,5-c]quinazolin-5- |
| | yl]amino}butanamide |
| | LC-MS (Method 2): Rt = 1.25 min; MS (ESIpos): m/z = 450 [M + H]⁺ |
| | ¹H-NMR (400 MHz, DMSO-d₆): δ [ppm] = 0.97 (t, 3H), 1.90-2.03 (m, 1H), |
| | 2.15-2.26 (m, 1H), 4.74 (td, 1H), 7.33-7.39 (m, 2H), 7.74 (s, 1H), 7.94- |
| | 7.99 (m, 1H), 8.05-8.11 (m, 3H), 8.32 (dd, 1H), 8.47 (d, 2H). |
| Example 564 | 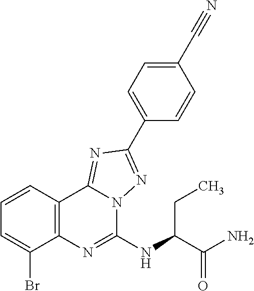 [SMILES: CC[C@H](Nc1nc2c(Br)cccc2c2nc(-c3ccc(C#N)cc3)nn12)C(N)=O] |
| | (2S)-2-{[7-bromo-2-(4-cyanophenyl)[1,2,4]triazolo[1,5-c]quinazolin-5- |
| | yl]amino}butanamide |
| | LC-MS (Method 2): Rt = 1.25 min; MS (ESIpos): m/z = 450 [M + H]⁺ |
| | ¹H-NMR (400 MHz, DMSO-d₆): δ [ppm] = 0.97 (t, 3H), 1.90-2.04 (m, 1H), |
| | 2.14-2.26 (m, 1H), 4.74 (td, 1H), 7.32-7.40 (m, 2H), 7.74 (s, 1H), 7.97 (d, |
| | 1H), 8.05-8.11 (m, 3H), 8.31 (dd, 1H), 8.47 (d, 2H). |
| Example 565 | 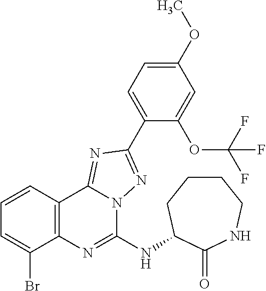 [SMILES: COc1ccc(-c2nc3c4cccc(Br)c4nc(N[C@@H]4CCCCNC4=O)n3n2)c(OC(F)(F)F)c1] |
| | (3R)-3-({7-bromo-2-[4-methoxy-2- |
| | (trifluoromethoxy)phenyl][1,2,4]triazolo[1,5-c]quinazolin-5- |
| | yl}amino)azepan-2-one |
| | LC-MS (Method 2): Rt = 1.56 min; MS (ESIpos): m/z = 565 [M + H]⁺ |
| | ¹H-NMR (400 MHz, DMSO-d₆): δ [ppm] = 1.27-1.40 (m, 1H), 1.46-1.58 (m, |
| | 1H), 1.82-2.11 (m, 3H), 3.14-3.24 (m, 1H), 3.27-3.36 (m, 1H and water |
| | signal), 3.91 (s, 3H), 4.81 (br dd, 1H), 7.11-7.13 (m, 1H), 7.24 (dd, 1H), |
| | 7.36 (t, 1H), 7.83 (d, 1H), 8.09 (dd, 1H), 8.21 (dd, 1H), 8.28 (dd, 1H), 8.34 |
| | (d, 1H). |
| Example 566 | 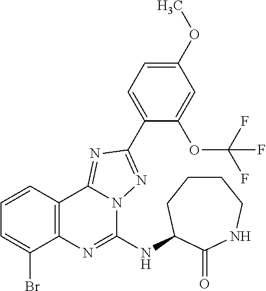 [SMILES: COc1ccc(-c2nc3c4cccc(Br)c4nc(N[C@H]4CCCCNC4=O)n3n2)c(OC(F)(F)F)c1] |
| | (3S)-3-({7-bromo-2-[4-methoxy-2- |
| | (trifluromethoxy)phenyl][1,2,4]triazolo[1,5-c]quinazolin-5- |
| | yl}amino)azepan-2-one |
| | LC-MS (Method 2): Rt = 1.56 min; MS (ESIpos): m/z = 565 [M + H]⁺ |
| | ¹H-NMR (400 MHz, DMSO-d₆): δ [ppm] = 1.27-1.40 (m, 1H), 1.46-1.57 (m, |
| | 1H), 1.82-2.10 (m, 3H), 3.14-3.24 (m, 1H), 3.27-3.34 (m, 1H and water |
| | signal), 3.91 (s, 3H), 4.81 (br dd, 1H), 7.11-7.13 (m, 1H), 7.24 (dd, 1H), |
| | 7.36 (t, 1H), 7.83 (d, 1H), 8.09 (dd, 1H), 8.21 (dd, 1H), 8.28 (dd, 1H), 8.34 |
| | (d, 1H). |
| Example 567 | 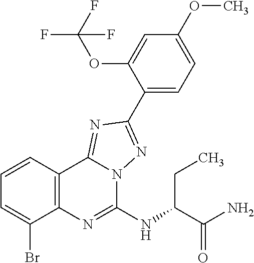 [SMILES: CC[C@@H](Nc1nc2c(Br)cccc2c2nc(-c3ccc(OC)cc3OC(F)(F)F)nn12)C(N)=O] |
| | (2R)-2-({7-bromo-2-[4-methoxy-2- |
| | (trifluoromethoxy)phenyl][1,2,4]triazolo[1,5-c]quinazolin-5- |
| | yl}amino)butanamide |
| | LC-MS (Method 2): Rt = 1.41 min; MS (ESIpos): m/z = 539 [M + H]⁺ |
| | ¹H-NMR (400 MHz, DMSO-d₆): δ [ppm] = 0.94 (t, 3H), 1.89-2.01 (m, 1H), |
| | 2.17-2.29 (m, 1H), 3.91 (s, 3H), 4.72-4.78 (m, 1H), 7.11-7.14 (m, 1H), |
| | 7.25 (dd, 1H), 7.35 (t, 1H), 7.40 (s, 1H), 7.62 (d, 1H), 7.81 (s, 1H), 8.08 (dd, |
| | 1H), 8.26-8.29 (m, 1H), 8.33 (s, 1H). |
| Example 568 | 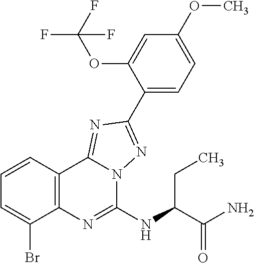 [SMILES: CC[C@H](Nc1nc2c(Br)cccc2c2nc(-c3ccc(OC)cc3OC(F)(F)F)nn12)C(N)=O] |
| | (2S)-2-({7-bromo-2-[4-methoxy-2- |
| | (trifluoromethoxy)phenyl][1,2,4]triazolo[1,5-c]quinazolin-5- |
| | yl}amino)butanamide |
| | LC-MS (Method 2): Rt = 1.43 min; MS (ESIpos): m/z = 539 [M + H]⁺ |
| | ¹H-NMR (400 MHz, DMSO-d₆): δ [ppm] = 0.94 (t, 3H), 1.88-2.01 (m, 1H), |
| | 2.17-2.29 (m, 1H), 3.91 (s, 3H), 4.72-4.78 (m, 1H), 7.11-7.14 (m, 1H), |
| | 7.24 (dd, 1H), 7.35 (t, 1H), 7.40 (s, 1H), 7.62 (d, 1H), 7.81 (s, 1H), 8.08 (dd, |
| | 1H), 8.27 (dd, 1H), 8.33 (d, 1H). |
| Example 569 | 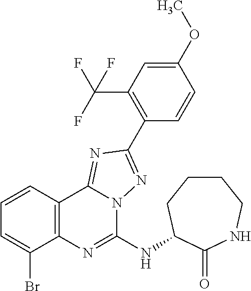 [SMILES: COc1ccc(-c2nc3c4cccc(Br)c4nc(N[C@@H]4CCCCNC4=O)n3n2)c(C(F)(F)F)c1] |
| | (3R)-3-({7-bromo-2-[4-methoxy-2- |
| | (trifluoromethyl)phenyl][1,2,4]triazolo[1,5-c]quinazolin-5- |
| | yl}amino)azepan-2-one |
| | LC-MS (Method 2): Rt = 1.50 min; MS (ESIpos): m/z = 549 [M + H]⁺ |
| | ¹H-NMR (400 MHz, DMSO-d₆): δ [ppm] = 1.27-1.40 (m, 1H), 1.47-1.59 (m, |
| | 1H), 1.81-2.10 (m, 3H), 3.14-3.23 (m, 1H), 3.94 (s, 3H), 4.82 (br dd, 1H), |
| | 7.36 (t, 1H), 7.41-7.47 (m, 2H), 7.83 (d, 1H), 8.01 (d, 1H), 8.10 (dd, 1H), |
| | 8.23 (br dd, 1H), 8.27 (dd, 1H). |
| Example 570 | 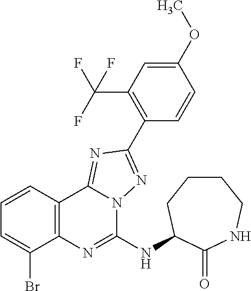 [SMILES: COc1ccc(-c2nc3c4cccc(Br)c4nc(N[C@H]4CCCCNC4=O)n3n2)c(C(F)(F)F)c1] |
| | (3S)-3-({7-bromo-2-[4-methoxy-2- |
| | (trifluoromethyl)phenyl][1,2,4]triazolo[1,5-c]quinazolin-5- |
| | yl}amino)azepan-2-one |
| | LC-MS (Method 2): Rt = 1.50 min; MS (ESIpos): m/z = 5549 [M + H]⁺ |
| | ¹H-NMR (400 MHz, DMSO-d₆): δ [ppm] = 1.27-1.40 (m, 1H), 1.47-1.59 (m, |
| | 1H), 1.82-2.10 (m, 3H), 3.13-3.23 (m, 1H), 3.94 (s, 3H), 4.82 (br dd, 1H), |
| | 7.36 (t, 1H), 7.41-7.47 (m, 2H), 7.83 (d, 1H), 8.01 (d, 1H), 8.10 (dd, 1H), |
| | 8.23 (br dd, 1H), 8.27 (dd, 1H). |
| Example 571 | 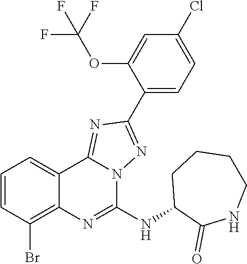 [SMILES: O=C1NCCCC[C@H]1Nc1nc2c(Br)cccc2c2nc(-c3ccc(Cl)cc3OC(F)(F)F)nn12] |
| | (3R)-3-({7-bromo-2-[4-chloro-2- |
| | (trifluoromethoxy)phenyl][1,2,4]triazolo[1,5-c]quinazolin-5- |
| | yl}amino)azepan-2-one |
| | LC-MS (Method 2): Rt = 1.69 min; MS (ESIpos): m/z = 569 [M + H]⁺ |
| | ¹H-NMR (400 MHz, CHLOROFORM-d): δ [ppm] = 1.48-1.61 (m, 1H), 1.65- |
| | 1.76 (m, 1H), 1.93-2.02 (m, 1H), 2.03-2.24 (m, 2H), 2.56-2.63 (m, 1H), |
| | 3.32-3.41 (m, 1H), 3.48 (ddd, 1H), 4.98 (ddd, 1H), 6.12 (br t, 1H), 7.26 (t, |
| | 1H), 7.45-7.50 (m, 2H), 7.80 (br d, 1H), 7.99 (dd, 1H), 8.36-8.41 (m, 2H). |
| Example 572 | 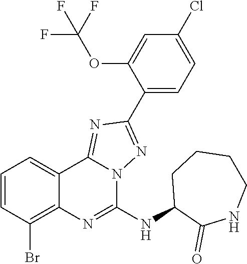 [SMILES: O=C1NCCCC[C@@H]1Nc1nc2c(Br)cccc2c2nc(-c3ccc(Cl)cc3OC(F)(F)F)nn12] |
| | (3S)-3-({7-bromo-2-[4-chloro-2- |
| | (trifluoromethoxy)phenyl][1,2,4]triazolo[1,5-c]quinazolin-5- |
| | yl}amino)azepan-2-one |
| | LC-MS (Method 2): Rt = 1.69 min; MS (ESIpos): m/z = 569 [M + H]⁺ |
| | ¹H-NMR (400 MHz, CHLOROFORM-d): δ [ppm] = 1.48-1.60 (m, 1H), 1.65- |
| | 1.76 (m, 1H), 1.93-2.02 (m, 1H), 2.03-2.24 (m, 2H), 2.56-2.64 (m, 1H), |
| | 3.32-3.41 (m, 1H), 3.48 (ddd, 1H), 4.98 (ddd, 1H), 6.13 (br t, 1H), 7.26 (t, |
| | 1H), 7.46-7.49 (m, 2H), 7.80 (br d, 1H), 7.99 (dd, 1H), 8.35-8.41 (m, 2H). |
| Example 573 | 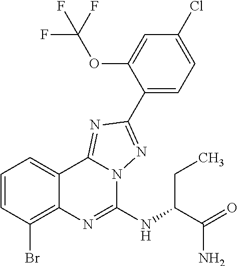 [SMILES: CC[C@@H](Nc1nc2c(Br)cccc2c2nc(-c3ccc(Cl)cc3OC(F)(F)F)nn12)C(N)=O] |
| | (2R)-2-({7-bromo-2-[4-chloro-2- |
| | (trifluoromethoxy)phenyl][1,2,4]triazolo[1,5-c]quinazolin-5- |
| | yl}amino)butanamide |
| | LC-MS (Method 2): Rt = 1.53 min; MS (ESIpos): m/z = 543 [M + H]⁺ |
| | ¹H-NMR (400 MHz, DMSO-d₆): δ [ppm] = 0.94 (t, 3H), 1.89-2.01 (m, 1H), |
| | 2.17-2.29 (m, 1H), 4.72-4.78 (m, 1H), 7.37 (t, 1H), 7.40 (s, 1H), 7.70 (d, |
| | 1H), 7.76-7.82 (m, 3H), 8.09 (dd, 1H), 8.28 (dd, 1H), 8.41 (d, 1H). |
| Example 574 | 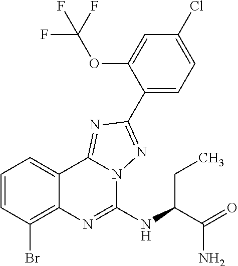 [SMILES: CC[C@H](Nc1nc2c(Br)cccc2c2nc(-c3ccc(Cl)cc3OC(F)(F)F)nn12)C(N)=O] |
| | (2S)-2-({7-bromo-2-[4-chloro-2- |
| | (trifluoromethoxy)phenyl][1,2,4]triazolo[1,5-c]quinazolin-5- |
| | yl}amino)butanamide |
| | LC-MS (Method 2): Rt = 1.50 min; MS (ESIpos): m/z = 543 [M + H]⁺ |
| | ¹H-NMR (400 MHz, DMSO-d₆): δ [ppm] = 0.94 (t, 3H), 1.89-2.01 (m, 1H), |
| | 2.17-2.29 (m, 1H), 4.72-4.78 (m, 1H), 7.34-7.42 (m, 2H), 7.70 (d, 1H), |
| | 7.76-7.83 (m, 3H), 8.09 (dd, 1H), 8.28 (dd, 1H), 8.42 (d, 1H). |
| Example 575 | 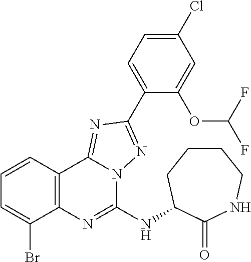 [SMILES: O=C1NCCCC[C@H]1Nc1nc2c(Br)cccc2c2nc(-c3ccc(Cl)cc3OC(F)F)nn12] |
| | (3R)-3-({7-bromo-2-[4-chloro-2- |
| | (difluoromethoxy)phenyl][1,2,4]triazolo[1,5-c]quinazolin-5- |
| | yl}amino)azepan-2-one |
| | LC-MS (Method 2): Rt = 1.55 min; MS (ESIpos): m/z = 551 [M + H]⁺ |
| | ¹H-NMR (400 MHz, DMSO-d₆): δ [ppm] = 1.27-1.41 (m, 1H), 1.47-1.59 (m, |
| | 1H), 1.81-2.10 (m, 3H), 2.40-2.48 (m, 1H and DMSO-signal), 3.14-3.24 |
| | (m, 1H), 3.26-3.34 (m, 1H and water signal), 4.82 (br dd, 1H), 7.17-7.57 |
| | (m, 3H), 7.60 (dd, 1H), 7.84 (d, 1H), 8.10 (dd, 1H), 8.22 (br dd, 1H), 8.26- |
| | 8.31 (m, 2H). |
| Example 576 | 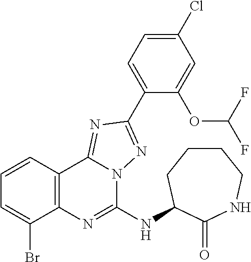 [SMILES: O=C1NCCCC[C@@H]1Nc1nc2c(Br)cccc2c2nc(-c3ccc(Cl)cc3OC(F)F)nn12] |
| | (3S)-3-({7-bromo-2-[4-chloro-2- |
| | (difluoromethoxy)phenyl][1,2,4]triazolo[1,5-c]quinazolin-5- |
| | yl}amino)azepan-2-one |
| | LC-MS (Method 2): Rt = 1.56 min; MS (ESIpos): m/z = 551 [M + H]⁺ |
| | ¹H-NMR (400 MHz, DMSO-d₆): δ [ppm] = 1.27-1.40 (m, 1H), 1.47-1.59 (m, |
| | 1H), 1.82-2.10 (m, 3H), 2.42-2.50 (m, 1H and DMSO signal), 3.14-3.24 |
| | (m, 1H), 3.27-3.33 (m, 1H and water signal), 4.83 (dd, 1H), 7.18-7.57 (m, |
| | 3H), 7.61 (dd, 1H), 7.84 (d, 1H), 8.10 (dd, 1H), 8.22 (dd, 1H), 8.27-8.31 (m, |
| | 2H). |
| Example 577 | 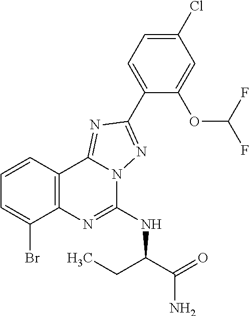 [SMILES: CC[C@@H](Nc1nc2c(Br)cccc2c2nc(-c3ccc(Cl)cc3OC(F)F)nn12)C(N)=O] |
| | (2R)-2-({7-bromo-2-[4-chloro-2- |
| | (difluoromethoxy)phenyl][1,2,4]triazolo[1,5-c]quinazolin-5- |
| | yl}amino)butanamide |
| | LC-MS (Method 2): Rt = 1.39 min; MS (ESIpos): m/z = 525 [M + H]⁺ |
| | ¹H-NMR (400 MHz, DMSO-d₆): δ [ppm] = 0.95 (t, 3H), 1.89-2.02 (m, 1H), |
| | 2.15-2.27 (m, 1H), 4.74 (td, 1H), 7.19-7.58 (m, 4H), 7.62 (dd, 1H), 7.73 (d, |
| | 1H), 7.77 (s, 1H), 8.09 (dd, 1H), 8.25-8.30 (m, 2H). |
| Example 578 | 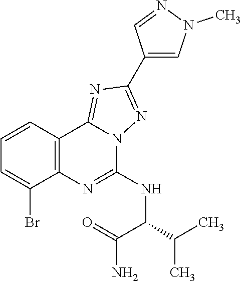 [SMILES: CC(C)[C@@H](Nc1nc2c(Br)cccc2c2nc(-c3cnn(C)c3)nn12)C(N)=O] |
| | N²-[7-bromo-2-(1-methyl-1H-pyrazol-4-yl)[1,2,4]triazolo[1,5-c]quinazolin- |
| | 5-yl]-D-valinamide |
| | LC-MS (Method 2): Rt = 1.08 min; MS (ESIpos): m/z = 443 [M + H]⁺ |
| | ¹H-NMR (400 MHz, DMSO-d₆): δ [ppm] = 0.98 (d, 3H), 1.07 (d, 3H), 2.34- |
| | 2.43 (m, 1H), 3.95 (s, 3H), 4.79 (dd, 1H), 7.30-7.36 (m, 2H), 7.45 (s, 1H), |
| | 7.85 (s, 1H), 8.05 (dd, 1H), 8.09 (s, 1H), 8.24 (dd, 1H), 8.52 (s, 1H). |
| Example 579 | 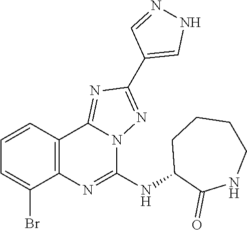 [SMILES: O=C1NCCCC[C@H]1Nc1nc2c(Br)cccc2c2nc(-c3cn[nH]c3)nn12] |
| | (3R)-3-{[7-bromo-2-(1H-pyrazol-4-yl)[1,2,4]triazolo[1,5-c]quinazolin-5- |
| | yl]amino}azepan-2-one |
| | LC-MS (Method 2): Rₜ = 1.13 min; MS (ESIneg): m/z = 439 [M − H]⁻ |
| | ¹H-NMR (400 MHz, DMSO-d₆): δ [ppm] = 12.7-1.40 (m, 1H), 1.47-1.59 (m, |
| | 1H), 1.81-2.10 (m, 3H), 2.39-2.46 (m, 1H), 3.14-3.23 (m, 1H), 4.83 (br dd, |
| | 1H), 7.33 (t, 1H), 7.71 (d, 1H), 8.06 (dd, 1H), 8.22-8.40 (m, 4H), 13.34 (br s, |
| | 1H). |
| Example 580 | 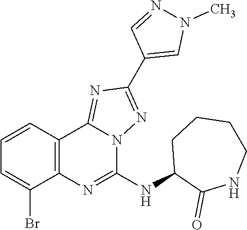 [SMILES: Cn1cc(-c2nc3c4cccc(Br)c4nc(N[C@H]4CCCCNC4=O)n3n2)cn1] |
| | (3S)-3-{[7-bromo-2-(1H-methyl-1H-pyrazol-4-yl)[1,2,4]triazolo[1,5- |
| | c]quinazolin-5-yl]amino}azepan-2-one |
| | LC-MS (Method 2): Rₜ = 1.18 min; MS (ESIpos): m/z = 455 [M + H]⁺ |
| | ¹H-NMR (400 MHz, DMSO-d₆): δ [ppm] = 1.26-1.40 (m, 1H), 1.47-1.58 (m, |
| | 1H), 1.82-1.99 (m, 2H), 2.03 (br s, 1H), 2.40-2.47 (m, 1H), 3.14-3.23 (m, |
| | 1H), 3.26-3.31 (m, 1H), 3.95 (s, 3H), 4.82 (dd, 1H), 7.33 (t, 1H), 7.70 (d, 1H), |
| | 8.05-8.09 (m, 2H), 8.21-8.27 (m, 2H), 8.50 (s, 1H). |
| Example 581 | 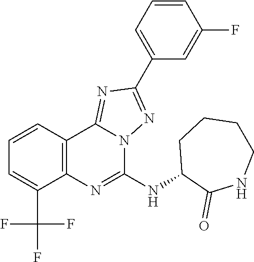 [SMILES: O=C1NCCCC[C@H]1Nc1nc2c(C(F)(F)F)cccc2c2nc(-c3cccc(F)c3)nn12] |
| | (3R)-3-{[2-(3-fluorophenyl)-7-(trifluoromethyl)[1,2,4]triazolo[1,5- |
| | c]quinazolin-5-yl]amino}azepan-2-one |
| | LC-MS (Method 2): Rₜ = 1.52 min; MS (ESIpos): m/z = 459 [M + H]⁺ |
| | ¹H-NMR (400 MHz, DMSO-d₆): δ [ppm] = 1.26-1.40 (m, 1H), 1.48-1.62 (m, |
| | 1H), 1.78-1.92 (m, 2H), 1.98-2.08 (m, 1H), 2.41 (br d, 1H), 3.14-3.28 (m, |
| | 2H), 4.77 (dd, 1H), 7.40-7.47 (m, 1H), 7.57 (t, 1H), 7.66 (td, 1H), 7.94 (d, |
| | 1H), 7.97-8.03 (m, 1H), 8.09-8.16 (m, 2H), 8.27 (br t, 1H), 8.56 (dd, 1H). |
| Example 582 | 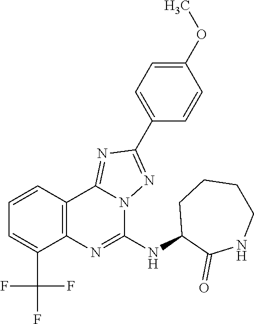 [SMILES: COc1ccc(-c2nc3c4cccc(C(F)(F)F)c4nc(N[C@H]4CCCCNC4=O)n3n2)cc1] |
| | (3S)-3-{[2-(4-methoxyphenyl)-7-(trifluoromethyl)[1,2,4]triazolo[1,5- |
| | c]quinazolin-5-yl]amino}azepan-2-one |
| | LC-MS (Method 2): Rₜ = 1.49 min; MS (ESIneg): m/z = 469 [M − H]⁻ |
| | ¹H-NMR (400 MHz, DMSO-d₆): δ [ppm] = 1.26-1.39 (m, 1H), 1.48-1.60 (m, |
| | 1H), 1.79-1.91 (m, 2H), 1.98-2.08 (m, 1H), 2.37-2.46 (m, 1H), 3.15-3.30 |
| | (m, 2H), 3.86 (s, 3H), 4.75 (br dd, 1H), 7.11-7.16 (m, 2H), 7.54 (t, 1H), 7.87 |
| | (d, 1H), 8.09 (d, 1H), 8.19-8.24 (m, 2H), 8.24-8.30 (m, 1H), 8.52-8.56 (m, |
| | 1H). |
| Example 583 | 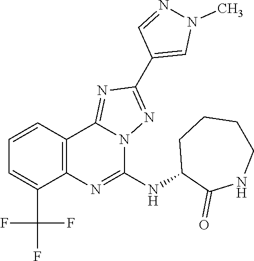 [SMILES: Cn1cc(-c2nc3c4cccc(C(F)(F)F)c4nc(N[C@@H]4CCCCNC4=O)n3n2)cn1] |
| | (3R)-3-{[2-(1-methyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 2): Rₜ = 1.21 min; MS (ESIpos): m/z = 445 [M + H]⁺ |
| | ¹H-NMR (400 MHz, DMSO-d₆): δ [ppm] = 1.171-1.37 (m, 2H), 1.73-1.86 (m, |
| | 2H), 1.90-1.99 (m, 1H), 2.30-2.37 (m, 1H), 3.10-3.16 (m, 2H), 3.98 (s, 3H), |
| | 4.59 (dd, 1H), 7.33 (d, 1H), 7.50 (t, 1H), 7.85 (s, 1H), 8.00 (dd, 1H), 8.13 (t, |
| | 1H), 8.27 (s, 1H), 8.56 (dd, 1H). |
| Example 584 | 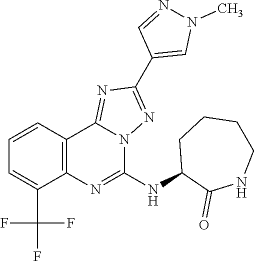 [SMILES: Cn1cc(-c2nc3c4cccc(C(F)(F)F)c4nc(N[C@H]4CCCCNC4=O)n3n2)cn1] |
| | (3S)-3-{[2-(1-methyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 2): Rₜ = 1.21 min; MS (ESIpos): m/z = 445 [M + H]⁺ |
| | ¹H-NMR (400 MHz, DMSO-d₆): δ [ppm] = 1.22-1.39 (m, 1H), 1.46-1.58 (m, |
| | 1H), 1.78-1.91 (m, 2H), 2.02 (br d, 1H), 2.33-2.44 (m, 1H), 3.15-3.32 (m, |
| | 2H), 3.95 (s, 3H), 4.74 (br dd, 1H), 7.54 (t, 1H), 7.77 (d, 1H), 8.07-8.11 (m, |
| | 2H), 8.26 (br t, 1H), 8.48-8.52 (m, 2H). |
| Example 585 | 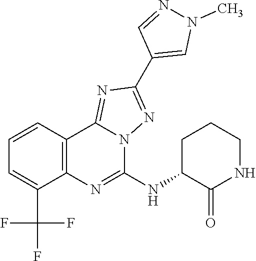 [SMILES: Cn1cc(-c2nc3c4cccc(C(F)(F)F)c4nc(N[C@@H]4CCCNC4=O)n3n2)cn1] |
| | (3R)-3-{[2-(1-methyl-1H-pyrazol-4-yl)-7- |
| | (trifluoromethyl)[1,2,4]triazolo[1,5-c]quinazolin-5-yl]amino}piperidin-2- |
| | one |
| | LC-MS (Method 2): Rₜ = 1.05 min; MS (ESIpos): m/z = 431 [M + H]⁺ |
| | ¹H-NMR (400 MHz, DMSO-d₆): δ [ppm] = 1.77-1.99 (m, 2H), 2.20-2.30 (m, |
| | 2H), 3.19-3.32 (m, 2H), 3.96 (s, 3H), 4.38-4.46 (m, 1H), 7.51 (t, 1H), 7.73- |
| | 7.79 (m, 1H), 8.04-8.09 (m, 2H), 8.42-8.50 (m, 3H). |
| Example 586 | 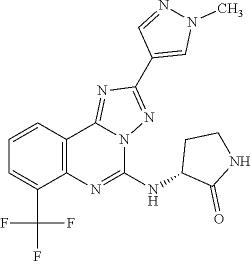 [SMILES: Cn1cc(-c2nc3c4cccc(C(F)(F)F)c4nc(N[C@@H]4CCNC4=O)n3n2)cn1] |
| | (3R)-3-{[2-(1-methyl-1H-pyrazol-4-yl)-7- |
| | (trifluoromethyl)[1,2,4]triazolo[1,5-c]quinazolin-5-yl]amino}pyrrolidin-2- |
| | one |
| | LC-MS (Method 2): Rₜ = 0.97 min; MS (ESIpos): m/z = 417 [M + H]⁺ |
| | ¹H-NMR (400 MHz, DMSO-d₆): δ [ppm] = 2.42-2.48 (m, 1H), 2.52-2.55 (m, |
| | 1H), 3.97 (s, 3H), 4.67-4.75 (m, 1H), 7.52 (t, 1H), 7.99 (s, 1H), 8.04-8.10 |
| | (m, 2H), 8.44 (s, 1H), 8.46-8.51 (m, 2H). |
| Example 587 | 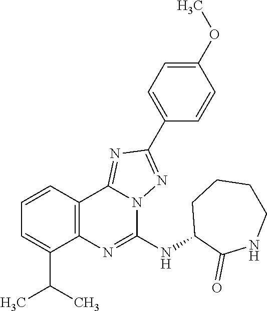 [SMILES: COc1ccc(-c2nc3c4cccc(C(C)C)c4nc(N[C@@H]4CCCCNC4=O)n3n2)cc1] |
| | (3R)-3-{[2-(4-methoxyphenyl)-7-(propan-2-yl)[1,2,4]triazolo[1,5- |
| | c]quinazolin-5-yl]amino}azepan-2-one |
| | LC-MS (Method 2): Rₜ = 1.55 min; MS (ESIpos): m/z = 445 [M + H]⁺ |
| | ¹H-NMR (400 MHz, DMSO-d₆): δ [ppm] = 1.33 (d, 3H), 1.39 (d, 3H), 1.48-1.62 |
| | (m, 1H), 1.81-1.95 (m, 2H), 1.99-2.09 (m, 1H), 2.37-2.46 (m, 1H), 3.13- |
| | 3.24 (m, 1H), 3.86 (s, 3H), 3.93-4.04 (m, 1H), 4.82 (dd, 1H), 7.11-7.16 (m, |
| | 2H), 7.41 (t, 1H), 7.59-7.68 (m, 2H), 8.15 (dd, 1H), 8.19-8.27 (m, 3H). |
| Example 588 | 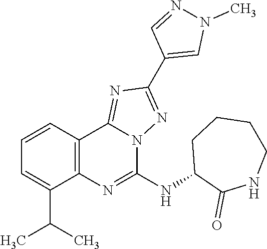 [SMILES: CC(C)c1cccc2c1nc(N[C@@H]1CCCCNC1=O)n1nc(-c3cnn(C)c3)nc21] |
| | (3R)-3-{[2-(1-methyl-1H-pyrazol-4-yl)-7-(propan-2-yl)[1,2,4]triazolo[1,5- |
| | c]quinazolin-5-yl]amino}azepan-2-one |
| | LC-MS (Method 2): Rₜ = 1.28 min; MS (ESIpos): m/z = 419 [M + H]⁺ |
| | ¹H-NMR (400 MHz, DMSO-d₆): δ [ppm] = 1.32 (d, 3H), 1.38 (d, 3H), 1.46-1.60 |
| | (m, 1H), 1.82-1.95 (m, 2H), 1.96-2.08 (m, 1H), 2.34-2.44 (m, 1H), 3.13- |
| | 3.22 (m, 1H), 3.93-4.02 (m, 4H), 4.81 (dd, 1H), 7.40 (t, 1H), 7.53 (d, 1H), |
| | 7.63 (dd, 1H), 8.06 (d, 1H), 8.10 (dd, 1H), 8.23 (dd, 1H), 8.49 (s, 1H). |
| Example 589 | 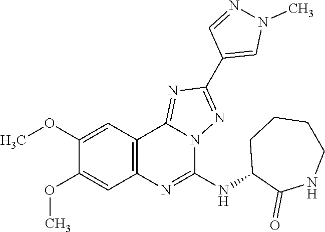 [SMILES: COc1cc2nc(N[C@@H]3CCCCNC3=O)n3nc(-c4cnn(C)c4)nc3c2cc1OC] |
| | (3R)-3-{[8,9-dimethoxy-2-(1-methyl-1H-pyrazol-4-yl)[1,2,4]triazolo[1,5- |
| | c]quinazolin-5-yl]amino}azepan-2-one |
| | LC-MS (Method 2): Rₜ = 0.95 min; MS (ESIpos): m/z = 437 [M + H]⁺ |
| | ¹H-NMR (400 MHz, DMSO-d₆): δ [ppm] = 1.25-1.38 (m, 1H), 1.48-1.60 (m, |
| | 1H), 1.81-1.95 (m, 2H), 1.97-2.06 (m, 1H), 2.28 (br d, 1H), 3.11-3.21 (m, |
| | 1H), 3.92 (s, 3H), 3.93 (s, 3H), 3.94 (s, 3H), 4.79 (dd, 1H), 7.11 (s, 1H), 7.43 |
| | (d, 1H), 7.53 (s, 1H), 8.05 (d, 1H), 8.19 (dd, 1H), 8.48 (s, 1H). |
| Example 590 | 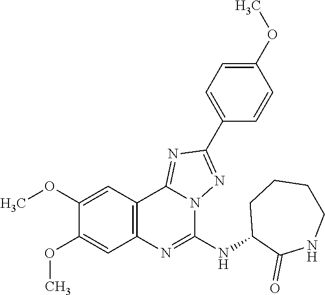 [SMILES: COc1ccc(-c2nc3c4cc(OC)c(OC)cc4nc(N[C@@H]4CCCCNC4=O)n3n2)cc1] |
| | (3R)-3-{[8,9-dimethoxy-2-(4-methoxyphenyl)[1,2,4]triazolo[1,5- |
| | c]quinazolin-5-yl]amino}azepan-2-one |
| | LC-MS (Method 2): Rₜ = 1.19 min; MS (ESIpos): m/z = 463 [M + H]⁺ |
| | ¹H-NMR (400 MHz, DMSO-d₆): δ [ppm] = 1.26-1.39 (m, 1H), 1.51-1.63 (m, |
| | 1H), 1.82-1.96 (m, 2H), 1.98-2.07 (m, 1H), 2.25-2.32 (m, 1H), 3.12-3.21 |
| | (m, 1H), 3.86 (s, 3H), 3.94 (s, 6H), 4.80 (br dd, 1H), 7.10-7.16 (m, 3H), 7.54 |
| | (d, 1H), 7.59 (s, 1H), 8.17-8.24 (m, 3H). |
| Example 591 | 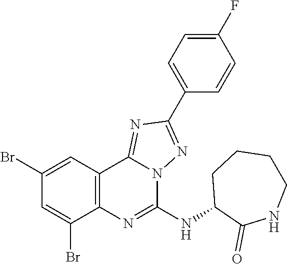 [SMILES: O=C1NCCCC[C@H]1Nc1nc2c(Br)cc(Br)cc2c2nc(-c3ccc(F)cc3)nn12] |
| | (3R)-3-{[7,9-dibromo-2-(4-fluorophenyl)[1,2,4]triazolo[1,5-c]quinazolin- |
| | 5-yl]amino}azepan-2-one |
| | LC-MS (Method 2): Rₜ = 1.71 min; MS (ESIpos): m/z = 549 [M + H]⁺ |
| | ¹H-NMR (400 MHz, DMSO-d₆): δ [ppm] = 1.28-1.40 (m, 1H), 1.50-1.62 (m, |
| | 1H), 1.82-1.99 (m, 2H), 2.01-2.10 (m, 1H), 2.38-2.45 (m, 1H), 3.14-3.24 |
| | (m, 1H), 4.82 (br dd, 1H), 7.41-7.48 (m, 2H), 7.92 (br d, 1H), 8.24-8.29 (m, |
| | 2H), 8.30-8.35 (m, 2H), 8.39 (d, 1H). |
| Example 592 | 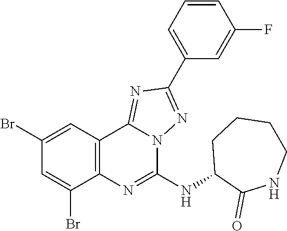 [SMILES: O=C1NCCCC[C@H]1Nc1nc2c(Br)cc(Br)cc2c2nc(-c3cccc(F)c3)nn12] |
| | (3R)-3-{[7,9-dibromo-2-(3-fluorophenyl)[1,2,4]triazolo[1,5-c]quinazolin- |
| | 5-yl]amino}azepan-2-one |
| | LC-MS (Method 2): Rt = 1.72 min; MS (ESIpos): m/z = 547 [M + H]⁺ |
| | ¹H-NMR (400 MHz, DMSO-d₆): δ [ppm] = 1.28-1.41 (m, 1H), 1.51-1.63 (m, |
| | 1H), 1.82-1.99 (m, 2H), 2.02-2.10 (m, 1H), 2.38-2.45 (m, 1H), 3.15-3.24 |
| | (m, 1H), 4.83 (br dd, 1H), 7.41-7.47 (m, 1H), 7.66 (td, 1H), 7.95 (br d, 1H), |
| | 7.99 (ddd, 1H), 8.12 (dt, 1H), 8.27 (d, 2H), 8.39 (d, 1H). |
| Example 593 | 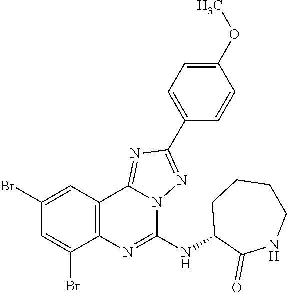 [SMILES: COc1ccc(-c2nc3c4cc(Br)cc(Br)c4nc(N[C@@H]4CCCCNC4=O)n3n2)cc1] |
| | (3R)-3-{[7,9-dibromo-2-(4-methoxyphenyl)[1,2,4]triazolo[1,5- |
| | c]quinazolin-5-yl]amino}azepan-2-one |
| | LC-MS (Method 2): Rₜ = 1.67 min; MS (ESIpos): m/z = 559 [M + H]⁺ |
| | ¹H-NMR (400 MHz, DMSO-d₆): δ [ppm] = 1.27-1.40 (m, 1H), 1.50-1.61 (m, |
| | 1H), 1.82-1.99 (m, 2H), 2.01-2.09 (m, 1H), 2.42 (br d, 1H), 3.14-3.24 (m, |
| | 1H), 3.86 (s, 3H), 4.82 (br dd, 1H), 7.12-7.17 (m, 2H), 7.88 (d, 1H), 8.19- |
| | 8.23 (m, 2H), 8.24-8.28 (m, 2H), 8.37 (d, 1H). |
| Example 594 | 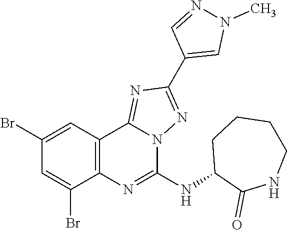 [SMILES: Cn1cc(-c2nc3c4cc(Br)cc(Br)c4nc(N[C@@H]4CCCCNC4=O)n3n2)cn1] |
| | (3R)-3-{[7,9-dibromo-2-(1-methyl-1H-pyrazol-4-yl)[1,2,4]triazolo[1,5- |
| | c]quinazolin-5-yl]amino}azepan-2-one |
| | LC-MS (Method 2): Rt = 1.40 min; MS (ESIpos): m/z = 533 [M + H]⁺ |
| | ¹H-NMR (400 MHz, DMSO-d₆): δ [ppm] = 1.26-1.39 (m, 1H), 1.47-1.59 (m, |
| | 1H), 1.82-2.09 (m, 3H), 2.37-2.44 (m, 1H), 3.13-3.23 (m, 1H), 3.95 (s, 3H), |
| | 4.80 (br dd, 1H), 7.77 (d, 1H), 8.07 (s, 1H), 8.23-8.28 (m, 2H), 8.31 (d, 1H), |
| | 8.49 (s, 1H). |

### Example 595

### (3R)-3-({2-[1-(oxetan-3-yl)-1H-pyrazol-4-yl][1,2,4]triazolo[1,5-c]quinazolin-5-yl}amino)azepan-2-one

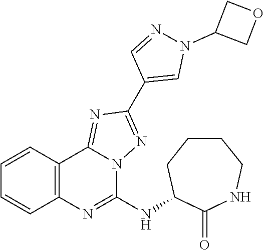 [SMILES: O=C1NCCCC[C@H]1Nc1nc2ccccc2c2nc(-c3cnn(C4COC4)c3)nn12]

(3R)-3-{[2-(1H-Pyrazol-4-yl)[1,2,4]triazolo[1,5-c]quinazolin-5-yl]amino}azepan-2-one (50.0 mg, 138 μmol), 3-bromooxetane (13 μL, 150 μmol) and cesium carbonate (135 mg, 414 μmol) were stirred in DMF at 100° C. for 1 h. The mixture was diluted with half sat. aqueous sodium chloride solution and extracted three times with EtOAc. The combined organic phases were dried over a silicone filter and concentrated under reduced pressure. The residue was purified by preparative HPLC to give 31.0 mg (95% purity, 51% yield) of the target compound.

LC-MS (Method 2): Rₜ=1.04 min; MS (ESIpos): m/z=419 [M+H]⁺

¹H-NMR (400 MHz, DMSO-d6): δ [ppm]=1.25-1.39 (m, 1H), 1.48-1.60 (m, 1H), 1.80-1.94 (m, 2H), 1.97-2.07 (m, 1H), 2.27-2.35 (m, 1H), 3.10-3.21 (m, 1H), 4.82 (br dd, 1H), 4.93-5.01 (m, 4H), 5.72 (quin, 1H), 7.44 (ddd, 1H), 7.62 (d, 1H), 7.64-7.68 (m, 1H), 7.73 (ddd m, 1H), 8.19-8.27 (m, 3H), 8.66 (s, 1H).

The following examples were prepared according to example 595:

| | Structure |
|---|---|
| | IUPAC-Name |
| | LC-MS (method): Retention time; Mass found |
| Example | ¹H-NMR |
| Example 596 | 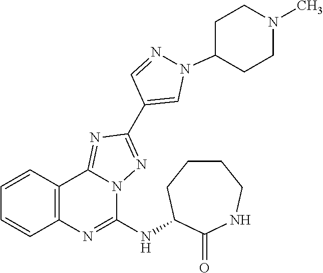 [SMILES: CN1CCC(n2cc(-c3nc4c5ccccc5nc(N[C@@H]5CCCCNC5=O)n4n3)cn2)CC1] |
| | (3R)-3-({2-[1-(1-methylpiperidin-4-yl)-1H-pyrazol-4-yl][1,2,4]triazolo[1,5- |
| | c]quinazolin-5-yl}amino)azepan-2-one |
| | LC-MS (Method 2): Rₜ = 1.10 min; MS (ESIpos): m/z = 460 [M + H]⁺ |
| | ¹H-NMR (400 MHz, DMSO-d₆): δ [ppm] = 1.24-1.38 (m, 1H), 1.48-1.60 (m, |
| | 1H), 1.80-1.94 (m,2H), 1.97-2.09 (m, 7H), 2.20 (s, 3H), 2.26-2.35 (m, 1H), |
| | 2.86 (brd, 2H), 3.11-3.20 (m, 1H), 4.18-4.28 (m, 1H), 4.81 (br dd, 1H),7.42 |
| | (ddd, 1H), 7.59 (d, 1H), 7.62-7.66 (m, 1H), 7.71 (ddd, 1H), 8.09 (d, 1H), 8.19- |
| | 8.26 (m, 2H), 8.51 (s, 1H). |
| Example 597 | 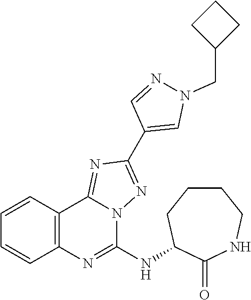 [SMILES: O=C1NCCCC[C@H]1Nc1nc2ccccc2c2nc(-c3cnn(CC4CCC4)c3)nn12] |
| | (3R)-3-({2-[1-(cyclobutylmethyl)-1H-pyrazol-4-yl][1,2,4]triazolo[1,5- |
| | c]quinazolin-5-yl}amino)azepan-2-one |
| | LC-MS (Method 2): Rₜ = 1.21 min; MS (ESIneg): m/z = 429 [M − H]⁻ |
| | ¹H-NMR (400 MHz, DMSO-d₆): δ [ppm] = 1.26-1.38 (m, 1H), 1.48-1.60 (m, |
| | 1H), 1.75-1.94 (m, 6H), 1.96-2.06 (m, 3H), 2.27-2.34 (m, 1H), 2.78-2.89 |
| | (m, 1H), 3.11-3.20 (m, 1H), 4.24 (d, 2H), 4.82 (br dd, 1H), 7.44 (ddd, 1H), |
| | 7.60 (d, 1H), 7.64-7.67 (m, 1H), 7.73 (ddd, 1H), 8.07 (d, 1H), 8.19-8.26 (m, |
| | 2H), 8.50 (s, 1H). |
| Example 598 | 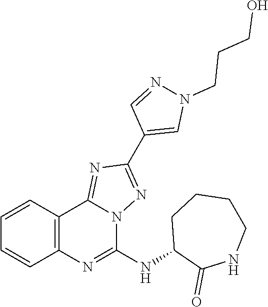 [SMILES: O=C1NCCCC[C@H]1Nc1nc2ccccc2c2nc(-c3cnn(CCCO)c3)nn12] |
| | (3R)-3-({2-[1-(3-hydroxypropyl)-1H-pyrazol-4-yl][1,2,4]triazolo[1,5- |
| | c]quinazolin-5-yl}amino)azepan-2-one |
| | LC-MS (Method 2): Rₜ = 0.99 min; MS (ESIpos): m/z = 421 [M + H]⁺ |
| | ¹H-NMR (400 MHz, DMSO-d₆): δ [ppm] = 1.25-1.38 (m, 1H), 1.48-1.60 (m, |
| | 1H), 1.81-1.94 (m, 2H), 1.94-2.06 (m, 3H), 2.27-2.35 (m, 1H), 3.10-3.20 |
| | (m, 1H), 3.41 (q, 2H), 4.28 (t, 2H), 4.65 (t, 1H), 4.83 (br dd, 1H), 7.41-7.46 |
| | (m, 1H), 7.61 (d, 1H), 7.64-7.68 (m, 1H), 7.72 (ddd, 1H), 8.09 (d, 1H), 8.18- |
| | 8.27 (m, 2H), 8.50 (s, 1H). |
| Example 599 | 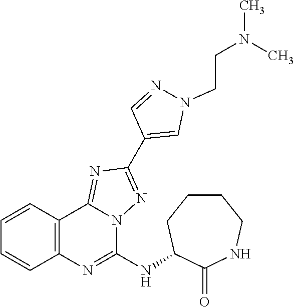 [SMILES: CN(C)CCn1cc(-c2nc3c4ccccc4nc(N[C@@H]4CCCCNC4=O)n3n2)cn1] |
| | (3R)-3-[(2-{1-[2-(dimethylamino)ethyl]-1H-pyrazol-4-yl}[1,2,4]triazolo[1,5- |
| | c]quinazolin-5-yl)amino]azepan-2-one |
| | LC-MS (Method 2): Rₜ = 1.23 min; MS (ESIpos): m/z = 398 [M + H]⁺ |
| | ¹H-NMR (400 MHz, DMSO-d₆): δ [ppm] = 1.26-1.38 (m, 1H), 1.48-1.60 (m, |
| | 1H), 1.81-1.95 (m, 2H), 1.98-2.06 (m, 1H), 2.20 (s, 6H), 2.28-2.35 (m, 1H), |
| | 2.71 (t, 2H), 3.11-3.20 (m, 1H), 4.31 (t, 2H), 4.82 (dd, 1H), 7.44 (ddd, 1H), |
| | 7.61 (d, 1H), 7.64-7.68 (m, 1H), 7.70-7.76 (m, 1H), 8.07 (s, 1H), 8.19-8.27 |
| | (m, 2H), 8.52 (d, 1H). |

### Example 600

### (3R)-3-({2-[1-(oxan-4-yl)-1H-pyrazol-4-yI][1,2,4]triazolo[1,5-c]quinazolin-5-yl}amino)azepan-2-one

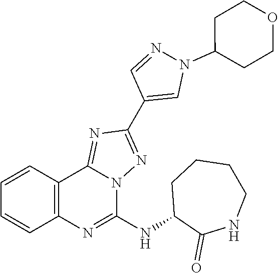 [SMILES: O=C1NCCCC[C@H]1Nc1nc2ccccc2c2nc(-c3cnn(C4CCOCC4)c3)nn12]

(3R)-3-[(2-Bromo[1,2,4]triazolo[1,5-c]quinazolin-5-yl)amino]azepan-2-one (75.0 mg, 200 μmol), 1-(oxan-4-yl)-4-(4,4,5,5-tetramethyl-1,3,2-dioxaborolan-2-yl)-1H-pyrazole (111 mg, 400 μmol) and XPhos Pd G1 (8.26 mg, 9.99 μmol) were solubilised in 1,4-dioxane (1.5 mL), water (300 μL) and potassium carbonate solution (300 μL, 2.0 M, 600 μmol). The mixture was sparged with argon and stirred for 1 h at 110° C. The mixture was evaporated and the residue was purified by preparative HPLC to give 59.2 mg (95% purity, 63% yield) of the target compound.

LC-MS (Method 2): Rₜ=1.10 m; MS (ESIpos): m/z=447 [M+H]⁺

¹H-NMR (400 MHz, DMSO-d6): δ [ppm]=1.25-1.39 (m, 1H), 1.48-1.60 (m, 1H), 1.80-1.94 (m, 2H), 1.97-2.09 (m, 5H), 2.27-2.35 (m, 1H), 3.10-3.21 (m, 1H), 3.43-3.55 (m, 2H), 3.99 (dt, 2H), 4.49-4.59 (m, 1H), 4.83 (br dd, 1H), 7.44 (ddd, 1H), 7.61 (d, 1H), 7.64-7.68 (m, 1H), 7.72 (ddd, 1H), 8.12 (d, 1H), 8.18-8.27 (m, 2H), 8.56 (s, 1H).

The following examples were prepared similarly to example 600:

| | Structure |
|---|---|
| | IUPAC-Name |
| | LC-MS (method): Retention time; Mass found |
| Example | ¹H-NMR |
| Example 601 | 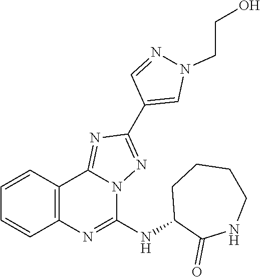 [SMILES: O=C1NCCCC[C@H]1Nc1nc2ccccc2c2nc(-c3cnn(CCO)c3)nn12] |
| | (3R)-3-({2-[1-(2-hydroxyethyl)-1H-pyrazol-4-yl][1,2,4]triazolo[1,5- |
| | c]quinazolin-5-yl}amino)azepan-2-one |
| | LC-MS (Method 2): Rₜ = 0.95 min; MS (ESIneg): m/z = 405 [M − H]⁻ |
| | ¹H-NMR (400 MHz, DMSO-d₆): δ [ppm] = 1.25-1.38 (m, 1H), 1.48-1.61 (m, |
| | 1H), 1.79-1.94 (m, 2H), 1.97-2.06 (m, 1H), 2.26-2.35 (m, 1H), 3.11-3.20 |
| | (m, 1H), 3.81 (q, 2H), 4.26 (t, 2H), 4.82 (br dd, 1H), 5.00 (t, 1H), 7.44 (ddd, |
| | 1H), 7.62 (d, 1H), 7.64-7.67 (m, 1H): 7.72 (ddd, 1H), 8.09 (d, 1H), 8.21 (dd, |
| | 1H), 8.25 (dd, 1H), 8.47 (s, 1H). |
| Example 602 | 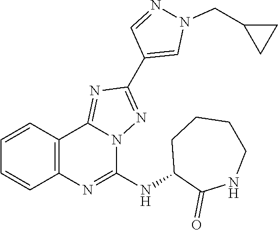 [SMILES: O=C1NCCCC[C@H]1Nc1nc2ccccc2c2nc(-c3cnn(CC4CC4)c3)nn12] |
| | (3R)-3-({2-[1-(cyclopropylmethyl)-1H-pyrazol-4-yl][1,2,4]triazolo[1,5- |
| | c]quinazolin-5-yl}amino)azepan-2-one |
| | LC-MS (Method 2): Rₜ = 1.13 min; MS (ESIpos): m/z = 417 [M + H]⁺ |
| | ¹H-NMR (400 MHz, DMSO-d₆): δ [ppm] = 0.41-0.46 (m, 2H), 0.54-0.60 (m, |
| | 2H), 1.25-1.40 (m, 2H), 1.47-1.60 (m, 1H), 1.80-1.95 (m, 2H), 1.98-2.06 |
| | (m, 1H), 2.26-2.34 (m, 1H), 3.11-3.21 (m, 1H), 4.08 (d, 2H), 4.83 (br dd, |
| | 1H), 7.44 (ddd, 1H), 7.61 (d, 1H), 7.64-7.68 (m, 1H), 7.72 (ddd, 1H), 8.09 (d, |
| | 1H), 8.21 (dd, 1H), 8.25 (dd, 1H), 8.54 (d, 1H). |
| Example 603 | 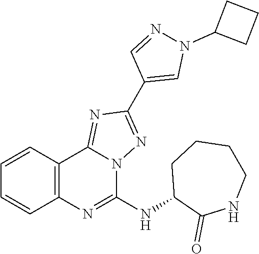 [SMILES: O=C1NCCCC[C@H]1Nc1nc2ccccc2c2nc(-c3cnn(C4CCC4)c3)nn12] |
| | (3R)-3-{[2-(1-cyclobutyl-1H-pyrazol-4-yl)[1,2,4]triazolo[1,5-c]quinazolin- |
| | 5-yl]amino}azepan-2-one |
| | LC-MS (Method 2): Rₜ = 1.22 min; MS (ESIpos): m/z = 417 [M + H]⁺ |
| | ¹H-NMR (400 MHz, DMSO-d₆): δ [ppm] = 1.26-1.39 (m, 1H), 1.48-1.60 (m, |
| | 1H), 1.76-1.94 (m, 4H), 1.97-2.07 (m, 1H), 2.26-2.35 (m, 1H), 2.37-2.47 |
| | (m, 2H), 2.53-2.63 (m, 2H), 3.11-3.21 (m, 1H), 3.34-3.41 (m, 1H), 4.83 (dd, |
| | 1H), 4.97 (quin, 1H), 7.44 (ddd, 1H), 7.60 (d, 1H), 7.64-7.68 (m, 1H), 7.73 |
| | (ddd, 1H), 8.12 (s, 1H), 8.19-8.27 (m, 2H), 8.59 (s, 1H). |
| Example 604 | 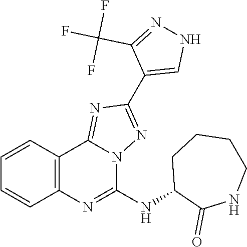 [SMILES: O=C1NCCCC[C@H]1Nc1nc2ccccc2c2nc(-c3c[nH]nc3C(F)(F)F)nn12] |
| | (3R)-3-({2-[3-(trifluoromethyl)-1H-pyrazol-4-yl][1,2,4]triazolo[1,5- |
| | c]quinazolin-5-yl}amino)azepan-2-one |
| | LC-MS (Method 2): Rₜ = 1.00 min; MS (ESIpos): m/z = 431 [M + H]⁺ |
| | ¹H-NMR (400 MHz, DMSO-d₆): δ [ppm] = 1.26-1.38 (m, 1H), 1.46-1.59 (m, |
| | 1H), 1.80-1.96 (m, 2H), 1.98-2.07 (m, 1H), 2.30-2.38 (m, 1H), 3.11-3.21 |
| | (m, 1H), 4.81 (dd, 1H), 7.46 (ddd, 1H), 7.63-7.70 (m, 2H), 7.72-7.78 (m, 1H), |
| | 8.20 (dd, 1H), 8.24 (dd, 1H), 8.73 (s, 1H), 14.08 (brs, 1H). |
| Example 605 | 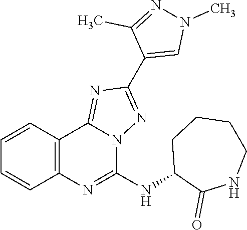 [SMILES: Cc1nn(C)cc1-c1nc2c3ccccc3nc(N[C@@H]3CCCCNC3=O)n2n1] |
| | (3R)-3-{[2-(1,3-dimethyl-1H-pyrazol-4-yl)[1,2,4]triazolo[1,5-c]quinazolin- |
| | 5-yl]amino}azepan-2-one |
| | LC-MS (Method 2): Rₜ = 1.11 min; MS (ESIpos): m/z = 391 [M + H]⁺ |
| | ¹H-NMR (400 MHz, DMSO-d₆): δ [ppm] = 1.26-1.38 (m, 1H), 1.48-1.60 (m, |
| | 1H), 1.80-1.95 (m, 2H), 1.97-2.07 (m, 1H), 2.28-2.37 (m, 1H), 2.58 (s, 3H), |
| | 3.11-3.21 (m, 1H), 3.34-3.40 (m, 1H), 3.86 (s, 3H), 4.81 (dd, 1H), 7.43 (ddd, |
| | 1H), 7.63-7.67 (m, 2H), 7.69-7.75 (m, 1H), 8.17-8.25 (m, 2H), 8.38 (s, 1H). |
| Example 606 | 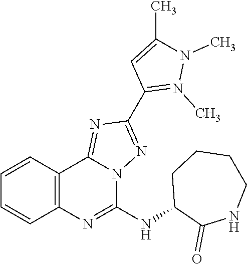 |
| | (3R)-3-{[2-(1,2,5-trimethyl-H-pyrrol-3-yl)[1,2,4]triazolo[1,5-c]quinazolin- |
| | 5-yl]amino}azepan-2-one |
| | LC-MS (Method 2): Rₜ = 1.24 min; MS (ESIpos): m/z = 404 [M + H]⁺ |
| | ¹H-NMR (400 MHz, DMSO-d₆): δ [ppm] = 1.25-1.38 (m, 1H), 1.48-1.61 (m, |
| | 1H), 1.80-1.94 (m, 2H), 1.98-2.06 (m, 1H), 2.22 (s, 3H), 2.28-2.36 (m, 1H), |
| | 2.70 (s, 3H), 3.11-3.20 (m, 1H), 3.45 (s, 3H), 4.81 (dd, 1H), 6.40 (d, 1H), 7.41 |
| | (ddd, 1H), 7.59 (d, 1H), 7.61-7.65 (m, 1H), 7.69 (ddd, 1H), 8.18 (dd, 1H), 8.23 |
| | (dd, 1H). |
| Example 607 | 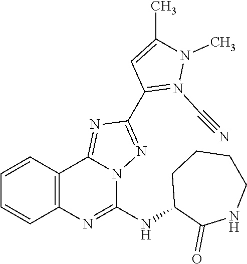 |
| | 1,5-dimethyl-3-(5-{[(3R)-2-oxoazepan-3-yl]amino}[1,2,4]triazolo[1,5- |
| | c]quinazolin-2-yl)-1H-pyrrole-2-carbonitrile |
| | LC-MS (Method 2): Rₜ = 1.23 min; MS (ESIpos): m/z = 415 [M + H]⁺ |
| | ¹H-NMR (400 MHz, DMSO-d₆): δ [ppm] = 1.26-1.39 (m, 1H), 1.48-1.59 (m, |
| | 1H), 1.81-1.95 (m, 2H), 1.98-2.07 (m, 1H), 2.28-2.38 (m, 4H), 3.11-3.21 |
| | (m, 1H), 3.72 (s, 3H), 4.82 (dd, 1H), 6.71 (d, 1H), 7.45 (ddd, 1H), 7.61 (d, 1H), |
| | 7.65-7.69 (m, 1H), 7.71-7.77 (m, 1H), 8.17-8.25 (m, 2H). |
| Example 608 | 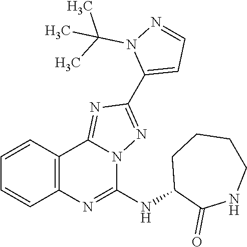 [SMILES: CC(C)(C)n1nccc1-c1nc2c3ccccc3nc(N[C@@H]3CCCCNC3=O)n2n1] |
| | (3R)-3-{[2-(1-tert-butyl-1H-pyrazol-5-yl)[1,2,4]triazolo[1,5-c]quinazolin-5- |
| | yl]amino}azepan-2-one |
| | LC-MS (Method 2): Rₜ = 1.25 min; MS (ESIpos): m/z = 419 [M + H]⁺ |
| | ¹H-NMR (400 MHz, DMSO-d₆): δ [ppm] = 1.25-1.38 (m, 1H), 1.49-1.60 (m, |
| | 1H), 1.63 (s, 9H), 1.80-1.95 (m, 2H), 1.97-2.07 (m, 1H), 2.31-2.39 (m, 1H), |
| | 3.10-3.21 (m, 1H), 4.81 (dd, 1H), 6.77 (d, 1H), 7.47 (ddd, 1H), 7.56 (d, 1H), |
| | 7.67-7.72 (m, 1H), 7.72-7.79 (m, 2H), 8.21 (dd, 1H), 8.26 (dd, 1H). |
| Example 609 | 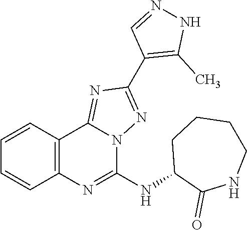 [SMILES: Cc1[nH]ncc1-c1nc2c3ccccc3nc(N[C@@H]3CCCCNC3=O)n2n1] |
| | (3R)-3-{[2-(5-methyl-1H-pyrazol-4-yl)[1,2,4]triazolo[1,5-c]quinazolin-5- |
| | yl]amino}azepan-2-one |
| | LC-MS (Method 2): Rt = 1.00 min; MS (ESIpos): m/z = 377 [M + H]⁺ |
| | ¹H-NMR (400 MHz, DMSO-d₆): δ [ppm] = 1.25-1.38 (m, 1H), 1.49-1.61 (m, |
| | 1H), 1.80-1.95 (m, 2H), 1.98-2.06 (m, 1H), 2.28-2.35 (m, 1H), 2.61 (br s, |
| | 1H), 2.69 (br s, 2H), 3.11-3.21 (m, 1H), 4.82 (dd, 1H), 7.43 (ddd, 1H), 7.63- |
| | 7.67 (m, 2H), 7.72 (ddd, 1H), 8.03 (br s, 1H), 8.18 (dd, 1H), 8.26 (br d, 1H), |
| | 12.93-13.08 (m, 1H). |
| Example 610 | 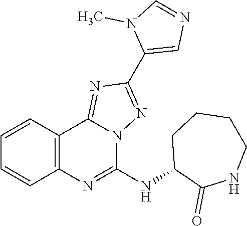 [SMILES: Cn1cncc1-c1nc2c3ccccc3nc(N[C@@H]3CCCCNC3=O)n2n1] |
| | (3R)-3-{[2-(1-methyl-1H-imidazol-5-yl)[1,2,4]triazolo[1,5-c]quinazolin-5- |
| | yl]amino}azepan-2-one |
| | LC-MS (Method 2): Rₜ = 1.00 min; MS (ESIpos): m/z = 377 [M + H]⁺ |
| | ¹H-NMR (400 MHz, DMSO-d₆): δ [ppm] = 1.23-1.38 (m, 1H), 1.51-1.63 (m, |
| | 1H), 1.80-1.94 (m, 2H), 1.97-2.07 (m, 1H), 2.28-2.35 (m, 1H), 3.09-3.27 |
| | (m, 1H), 4.11 (s, 3H), 4.83 (br d, 1H), 7.45 (ddd, 1H), 7.65-7.68 (m, 1H), 7.70- |
| | 7.77 (m, 3H), 7.91 (s, 1H), 8.19 (dd, 1H), 8.28 (dd, 1H). |
| Example 611 | 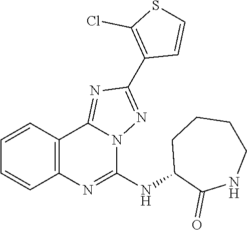 [SMILES: O=C1NCCCC[C@H]1Nc1nc2ccccc2c2nc(-c3ccsc3Cl)nn12] |
| | (3R)-3-{[2-(2-chlorothiophen-3-yl)[1,2,4]triazolo[1,5-c]quinazolin-5- |
| | yl]amino}azepan-2-one |
| | LC-MS (Method 2): Rₜ = 1.37 min; MS (ESIpos): m/z = 413 [M + H]⁺ |
| | ¹H-NMR (400 MHz, DMSO-d₆): δ [ppm] = 1.25-1.39 (m, 1H), 1.49-1.61 (m, |
| | 1H), 1.80-1.95 (m, 2H), 1.98-2.07 (m, 1H), 2.29-2.37 (m, 1H), 3.12-3.21 |
| | (m, 1H), 4.83 (dd, 1H), 7.46 (ddd, 1H), 7.66-7.78 (m, 5H), 8.20 (dd, 1H), 8.28 |
| | (dd, 1H). |
| Example 612 | 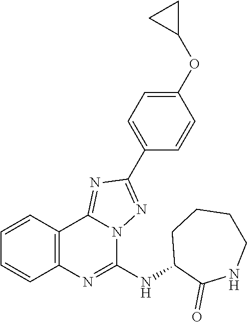 [SMILES: O=C1NCCCC[C@H]1Nc1nc2ccccc2c2nc(-c3ccc(OC4CC4)cc3)nn12] |
| | (3R)-3-({2-[4-(cyclopropyloxy)phenyl][1,2,4]triazolo[1,5-c]quinazolin-5- |
| | yl}amino)azepan-2-one |
| | LC-MS (Method 2): Rₜ = 1.27 min; MS (ESIpos): m/z = 429 [M + H]⁺ |
| | ¹H-NMR (400 MHz, DMSO-d₆): δ [ppm] = 0.69-0.76 (m, 2H), 0.81-0.88 (m, |
| | 2H), 1.25-1.41 (m, 1H), 1.47-1.64 (m, 1H), 1.79-1.96 (m, 2H), 1.97-2.09 |
| | (m, 1H), 2.27-2.37 (m, 1H), 3.11-3.23 (m, 1H), 3.35-3.44 (m, 1H), 3.96 (tt, |
| | 1H), 4.78-4.88 (m, 1H), 7.23-7.31 (m, 2H), 7.45 (ddd, 1H), 7.63-7.69 (m, |
| | 1H), 7.70-7.77 (m, 2H), 8.17-8.26 (m, 3H), 8.30 (dd, 1H). |
| Example 613 | 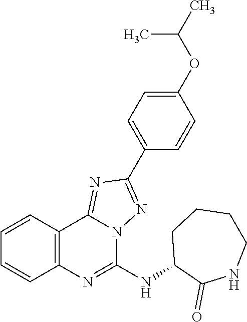 [SMILES: CC(C)Oc1ccc(-c2nc3c4ccccc4nc(N[C@@H]4CCCCNC4=O)n3n2)cc1] |
| | (3R)-3-[(2-{4-[(propan-2-yl)oxy]phenyl}[1,2,4]triazolo[1,5-c]quinazolin-5- |
| | yl)amino]azepan-2-one |
| | LC-MS (Method 2): Rₜ = 1.42 min; MS (ESIpos): m/z = 431 [M + H]⁺ |
| | ¹H-NMR (500 MHz, DMSO-d₆): δ [ppm] = 1.27-1.37 (m, 7H), 1.51 1.62 (m, |
| | 1H), 1.79-1.95 (m, 2H), 1.97-2.08 (m, 1H), 2.28-2.35 (m, 1H), 3.10-3.24 |
| | (m, 1H), 3.35-3.42 (m, 1H), 4.74 (spt, 1H), 4.80-4.87 (m, 1H), 7.09-7.13 |
| | (m, 2H), 7.45 (ddd, 1H), 7.64-7.68 (m, 1H), 7.70-7.76 (m, 2H), 8.16-8.24 |
| | (m, 3H), 8.29 (dd, 1H). |
| Example 614 | 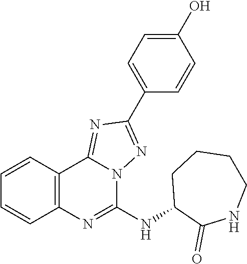 [SMILES: O=C1NCCCC[C@H]1Nc1nc2ccccc2c2nc(-c3ccc(O)cc3)nn12] |
| | (3R)-3-{[2-(4-hydroxyphenyl)[1,2,4]triazolo[1,5-c]quinazolin-5- |
| | yl]amino}azepan-2-one |
| | LC-MS (Method 2): Rₜ = 0.85 min; MS (ESIpos): m/z = 389 [M + H]⁺ |
| | ¹H-NMR (400 MHz, DMSO-d₆): δ [ppm] = 1.22-1.43 (m, 1H), 1.44-1.62 (m, |
| | 1H), 1.80-1.98 (m, 2H), 1.98-2.08 (m, 1H), 2.28-2.37 (m, 1H), 3.10-3.21 |
| | (m, 1H), 3.35-3.41 (m, 1H), 4.83 (br dd, 1H), 6.93-6.97 (m, 2H), 7.44 (ddd, |
| | 1H), 7.64-7.75 (m, 3H), 8.09-8.14 (m, 2H), 8.22 (dd, 1H), 8.28 (dd, 1H), |
| | 10.01 (s, 1H). |
| Example 615 | 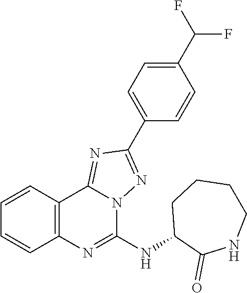 [SMILES: O=C1NCCCC[C@H]1Nc1nc2ccccc2c2nc(-c3ccc(C(F)F)cc3)nn12] |
| | (3R)-3-({2-[4-(difluoromethyl)phenyl][1,2,4]triazolo[1,5-c]quinazolin-5- |
| | yl}amino)azepan-2-one |
| | LC-MS (Method 2): Rₜ = 1.34 min; MS (ESIpos): m/z = 423 [M + H]⁺ |
| | ¹H-NMR (400 MHz, DMSO-d₆): δ [ppm] = 1.27-1.39 (m, 1H), 1.52-1.63 (m, |
| | 1H), 1.82-1.95 (m, 2H), 1.99-2.07 (m, 1H), 2.30-2.36 (m, 1H), 3.13-3.21 |
| | (m, 1H), 3.35-3.41 (m, 1H), 4..84 (br dd, 1H), 7.17 (t, 1H), 7.47 (ddd, 1H), |
| | 7.66-7.71 (m, 1H), 7.72-7.84 (m, 4H), 8.23 (dd, 1H), 8.32 (dd, 1H), 8.43 (d, |
| | 2H). |
| Example 616 | 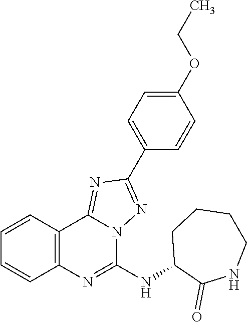 [SMILES: CCOc1ccc(-c2nc3c4ccccc4nc(N[C@@H]4CCCCNC4=O)n3n2)cc1] |
| | (3R)-3-{[2-(4-ethoxyphenyl)[1,2,4]triazolo[1,5-c]quinazolin-5- |
| | yl]amino}azepan-2-one |
| | LC-MS (Method 2): Rₜ = 1.38 min; MS (ESIpos): m/z = 417 [M + H]⁺ |
| | ¹H-NMR (400 MHz, DMSO-d₆): δ [ppm] = 1.26-1.40 (m, 4H), 1.47-1.63 (m, |
| | 1H), 1.80-1.98 (m, 2H), 1.98-2.07 (m, 1H), 2.29-2.36 (m, 1H), 3.12-3.21 |
| | (m, 1H), 3.35-3.41 (m, 1H), 4.13 (q, 2H), 4.83 (dd, 1H), 7.10-7.14 (m, 2H), |
| | 7.45 (ddd, 1H), 7.64-7.68 (m, 1H), 7.69-7.76 (m, 2H), 8.18-8.24 (m, 3H), |
| | 8.29 (dd, 1H). |
| Example 617 | 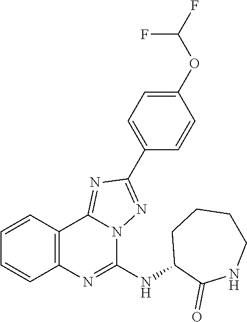 [SMILES: O=C1NCCCC[C@H]1Nc1nc2ccccc2c2nc(-c3ccc(OC(F)F)cc3)nn12] |
| | (3R)-3-({2-[4-(difluoromethoxy)phenyl][1,2,4]triazolo[1,5-c]quinazolin-5- |
| | yl}amino)azepan-2-one |
| | LC-MS (Method 2): Rₜ = 1.34 min; MS (ESIpos): m/z = 439 [M + H]⁺ |
| | ¹H-NMR (400 MHz, DMSO-d₆): δ [ppm] = 1.27-1.40 (m, 1H), 1.51-1.63 (m, |
| | 1H), 1.79-1.95 (m, 2H), 1.98-2.08 (m, 1H), 2.27-2.36 (m, 1H), 3.12-3.23 |
| | (m, 1H), 4.83 (br dd, 1H), 7.39 (t, 1H), 7.36-7.42 (m, 2H), 7.43-7.49 (m, 1H), |
| | 7.64-7.69 (m, 1H), 7.71-7.77 (m, 2H), 8.22 (dd, 1H), 8.30 (dd, 1H), 8.31- |
| | 8.36 (m, 2H). |
| Example 618 | 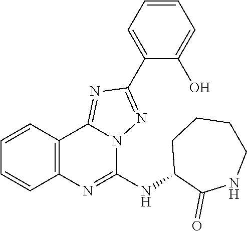 [SMILES: O=C1NCCCC[C@H]1Nc1nc2ccccc2c2nc(-c3ccccc3O)nn12] |
| | (3R)-3-{[2-(2-hydroxyphenyl)[1,2,4]triazolo[1,5-c]quinazolin-5- |
| | yl]amino}azepan-2-one |
| | LC-MS (Method 2): Rₜ = 1.35 min; MS (ESIpos): m/z = 389 [M + H]⁺ |
| | ¹H-NMR (500 MHz, DMSO-d₆): δ [ppm] = 1.27-1.40 (m, 1H), 1.57-1.71 (m, |
| | 1H), 1.80-1.93 (m, 2H), 1.97-2.08 (m, 1H), 2.28 (br d, 1H), 3.09-3.24 (m, |
| | 1H), 4.88 (br d, 1H), 6.99-7.09 (m, 2H), 7.40-7.52 (m, 2H), 7.68 (d, 1H), 7.76 |
| | (ddd, 1H), 8.03 (br s, 1H), 8.16 (dd, 1H), 8.20 (dd, 1H), 8.36 (dd, 1H). |
| Example 619 | 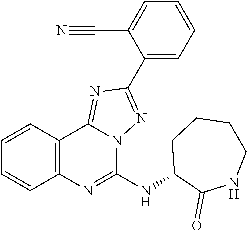 [SMILES: N#Cc1ccccc1-c1nc2c3ccccc3nc(N[C@@H]3CCCCNC3=O)n2n1] |
| | 2-(5-{[(3R)-2-oxoazepan-3-yl]amino}[1,2,4]triazolo[1,5-c]quinazolin-2- |
| | yl)benzonitrile |
| | LC-MS (Method 2): Rₜ = 1.23 min; MS (ESIpos): m/z = 398 [M + H]⁺ |
| | ¹H-NMR (400 MHz, DMSO-d₆): δ [ppm] = 1.22-1.39 (m, 1H), 1.47-1.64 (m, |
| | 1H), 1.82-1.99 (m, 2H), 1.99-2.08 (m, 1H), 2.31-2.46 (m, 1H), 3.12-3.30 |
| | (m, 1H), 4.84 (br dd, 1H), 7.49 (ddd, 1H), 7.67-7.73 (m, 1H), 7.73-7.82 (m, |
| | 3H), 7.94 (td, 1H), 8.08 (dd, 1H), 8.20 (dd, 1H), 8.30 (dd, 1H), 8.40 (dd, 1H). |
| Example 620 | 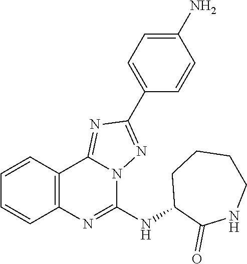 [SMILES: Nc1ccc(-c2nc3c4ccccc4nc(N[C@@H]4CCCCNC4=O)n3n2)cc1] |
| | (3R)-3-{[2-(4-aminophenyl)[1,2,4]triazolo[1,5-c]quinazolin-5- |
| | yl]amino}azepan-2-one |
| | LC-MS (Method 2): Rₜ = 1.01 min; MS (ESIpos): m/z = 388 [M + H]⁺ |
| | ¹H-NMR (400 MHz, DMSO-d₆): δ [ppm] = 1.25-1.42 (m, 1H), 1.47-1.62 (m, |
| | 1H), 1.78-1.96 (m, 2H), 1.97-2.08 (m, 1H), 2.26-2.36 (m, 1H), 3.09-3.24 |
| | (m, 1H), 3.35-3.42 (m, 1H), 4.82 (dd, 1H), 5.67 (s, 2H), 6.67-6.73 (m, 2H), |
| | 7.43 (ddd, 1H), 7.62-7.67 (m, 2H), 7.68-7.74 (m, 1H), 7.93-7.98 (m, 2H), |
| | 8.21 (dd, 1H), 8.26 (dd, 1H). |
| Example 621 | 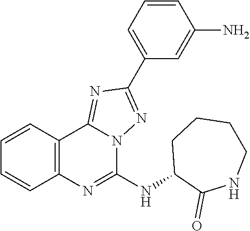 [SMILES: Nc1cccc(-c2nc3c4ccccc4nc(N[C@@H]4CCCCNC4=O)n3n2)c1] |
| | (3R)-3-{[2-(3-aminophenyl)[1,2,4]triazolo[1,5-c]quinazolin-5- |
| | yl]amino}azepan-2-one |
| | LC-MS (Method 2): Rₜ = 1.11 min; MS (ESIpos): m/z = 388 [M + H]⁺ |
| | ¹H-NMR (400 MHz, DMSO-d₆): δ [ppm] = 1.26-1.39 (m, 1H), 1.49-1.62 (m, |
| | 1H), 1.81-1.96 (m, 2H), 1.99-2.06 (m, 1H), 2.27-2.38 (m, 1H), 3.11-3.22 |
| | (m, 1H), 3.35-3.42 (m, 1H), 4.83 (dd, 1H), 5.41 (s, 2H), 6.72 (ddd, 1H), 7.21 |
| | (t, 1H), 7.41-7.49 (m, 2H), 7.55 (t, 1H), 7.65-7.70 (m, 2H), 7.70-7.76 (m, |
| | 1H), 8.23 (dd, 1H), 8.28 (dd, 1H). |
| Example 622 | 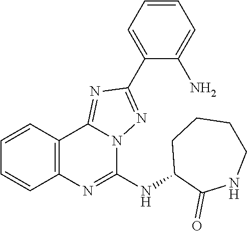 [SMILES: Nc1ccccc1-c1nc2c3ccccc3nc(N[C@@H]3CCCCNC3=O)n2n1] |
| | (3R)-3-{[2-(2-aminophenyl)[1,2,4]triazolo[1,5-c]quinazolin-5- |
| | yl]amino}azepan-2-one |
| | LC-MS (Method 2): Rₜ = 1.24 min; MS (ESIpos): m/z = 388 [M + H]⁺ |
| | ¹H-NMR (500 MHz, DMSO-d₆): δ [ppm] = 1.26-1.39 (m, 1H), 1.63-1.75 (m, |
| | 1H), 1.79-1.93 (m, 2H), 1.96-2.13 (m, 1H), 2.24 (br d, 1H), 3.11-3.21 (m, |
| | 1H), 3.35-3.42 (m, 1H), 4.89 (dd, 1H), 6.69 (ddd, 1H), 6.74 (s, 2H), 6.89 (dd, |
| | 1H), 7.21 (ddd, 1H), 7.44 (ddd, 1H), 7.63-7.67 (m, 1H), 7.73 (ddd, 1H), 8.06 |
| | (d, 1H), 8.09 (dd, 1H), 8.22 (dd, 1H), 8.32 (dd, 1H). |
| Example 623 | 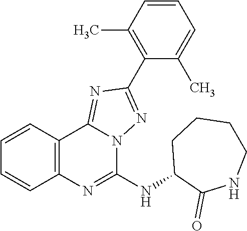 [SMILES: Cc1cccc(C)c1-c1nc2c3ccccc3nc(N[C@@H]3CCCCNC3=O)n2n1] |
| | (3R)-3-{[2-(2,6-dimethylphenyl)[1,2,4]triazolo[1,5-c]quinazolin-5- |
| | yl]amino}azepan-2-one |
| | LC-MS (Method 2): Rₜ = 1.31 min; MS (ESIpos): m/z = 401 [M + H]⁺ |
| | ¹H-NMR (400 MHz, DMSO-d₆): δ [ppm] = 1.22-1.37 (m, 1H), 1.49-1.61 (m, |
| | 1H), 1.80-1.95 (m, 2H), 1.96-2.13 (m, 1H), 2.15 (s, 6H), 2.31-2.39 (m, 1H), |
| | 3.10-3.20 (m, 1H), 4.81 (dd, 1H), 7.20 (d, 2H), 7.30-7.36 (m, 1H), 7.45 (ddd, |
| | 1H), 7.68-7.77 (m, 3H), 8.20 (dd, 1H), 8.26 (dd, 1H). |
| Example 624 | 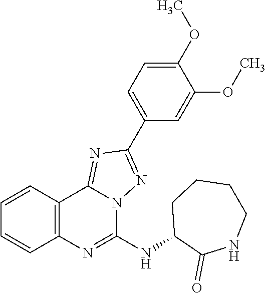 [SMILES: COc1ccc(-c2nc3c4ccccc4nc(N[C@@H]4CCCCNC4=O)n3n2)cc1OC] |
| | (3R)-3-{[2-(3,4-dimethoxyphenyl)[1,2,4]triazolo[1,5-c]quinazolin-5- |
| | yl]amino}azepan-2-one |
| | LC-MS (Method 2): Rₜ = 1.22 min; MS (ESIpos): m/z = 433 [M + H]⁺ |
| | ¹H-NMR (400 MHz, DMSO-d₆): δ [ppm] = 1.25-1.41 (m, 1H), 1.51-1.65 (m, |
| | 1H), 1.78-1.96 (m, 2H), 1.97-2.07 (m, 1H), 2.25-2.36 (m, 1H), 3.09-3.24 |
| | (m, 1H), 3.36-3.42 (m, 1H), 3.86 (s, 3H), 3.91 (s, 3H), 4.85 (brdd, 1H), 7.17 |
| | (d, 1H), 7.45 (ddd, 1H), 7.63-7.68 (m, 1H), 7.69-7.76 (m, 2H), 7.78 (d, 1H), |
| | 7.88 (dd, 1H), 8.19 (dd, 1H), 8.30 (dd, 1H). |
| Example 625 | 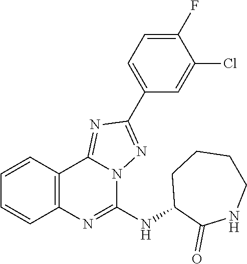 [SMILES: O=C1NCCCC[C@H]1Nc1nc2ccccc2c2nc(-c3ccc(F)c(Cl)c3)nn12] |
| | (3R)-3-{[2-(3-chloro-4-fluorophenyl)[1,2,4]triazolo[1,5-c]quinazolin-5- |
| | yl]amino}azepan-2-one |
| | LC-MS (Method 2): Rₜ = 1.46 min; MS (ESIpos): m/z = 425 [M + H]⁺ |
| | ¹H-NMR (400 MHz, DMSO-d₆): δ [ppm] = 1.25-1.38 (m, 1H), 1.47-1.71 (m, |
| | 1H), 1.76-1.94 (m, 2H), 1.95-2.07 (m, 1H), 2.24-2.33 (m, 1H), 3.06-3.23 |
| | (m, 1H), 4.84 (dd, 1H), 7.47 (ddd, 1H), 7.63-7.70 (m, 2H), 7.72-7.76 (m, 1H), |
| | 7.78 (d, 1H), 8.21 (dd, 1H), 8.26-8.33 (m, 2H), 8.39 (dd, 1H). |
| Example 626 | 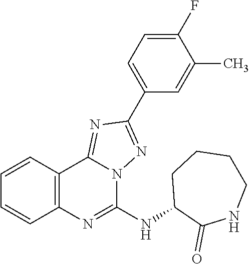 [SMILES: Cc1cc(-c2nc3c4ccccc4nc(N[C@@H]4CCCCNC4=O)n3n2)ccc1F] |
| | (3R)-3-{[2-(4-fluoro-3-methylphenyl)[1,2,4]triazolo[1,5-c]quinazolin-5- |
| | yl]amino}azepan-2-one |
| | LC-MS (Method 2): Rₜ = 1.41 min; MS (ESIpos): m/z = 405 [M + H]⁺ |
| | ¹H-NMR (500 MHz, DMSO-d₆): δ [ppm] = 1.26-1.39 (m, 1H), 1.51-1.62 (m, |
| | 1H), 1.80-1.94 (m, 2H), 1.99-2.07 (m, 1H), 2.27-2.35 (m, 1H), 2.38 (s, 3H), |
| | 3.10-3.23 (m, 1H), 3.35-3.42 (m, 1H), 4.80-4.88 (m, 1H), 7.36 (t, 1H), 7.46 |
| | (td, 1H), 7.65-7.69 (m, 1H), 7.70-7.78 (m, 2H), 8.15 (ddd, 1H), 8.19-8.24 |
| | (m, 2H), 8.30 (dd, 1H). |
| Example 627 | 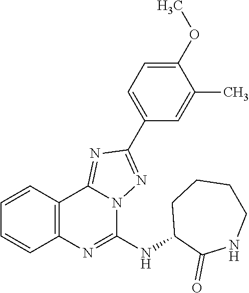 [SMILES: COc1ccc(-c2nc3c4ccccc4nc(N[C@@H]4CCCCNC4=O)n3n2)cc1C] |
| | (3R)-3-{[2-(4-methoxy-3-methylphenyl)[1,2,4]triazolo[1,5-c]quinazolin-5- |
| | yl]amino}azepan-2-one |
| | LC-MS (Method 2): Rₜ = 1.36 min; MS (ESIpos): m/z = 417 [M + H]⁺ |
| | ¹H-NMR (400 MHz, DMSO-d₆): δ [ppm] = 1.25-1.41 (m, 1H), 1.49-1.63 (m, |
| | 1H), 1.80-1.96 (m,2H), 1.97-2.10 (m, 1H), 2.30-2.39 (m, 1H), 2.74 (s, 3H), |
| | 3.09-3.24 (m, 1H), 3.35-3.42 (m, 1H), 3.84 (s, 3H), 4.82 (dd, 1H), 6.94-7.01 |
| | (m, 2H), 7.45 (ddd, 1H), 7.65-7.69 (m, 1H), 7.70-7.77 (m, 2H), 8.13 (d, 1H), |
| | 8.19 (dd, 1H), 8.29 (dd, 1H). |
| Example 629 | 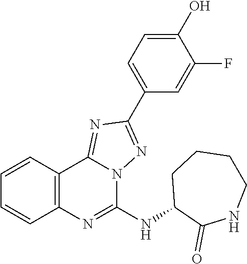 [SMILES: O=C1NCCCC[C@H]1Nc1nc2ccccc2c2nc(-c3ccc(O)c(F)c3)nn12] |
| | (3R)-3-{[2-(3-fluoro-4-hydroxyphenyl)[1,2,4]triazolo[1,5-c]quinazolin-5- |
| | yl]amino}azepan-2-one |
| | LC-MS (Method 2): Rₜ = 0.71 min; MS (ESIpos): m/z = 407 [M + H]⁺ |
| | ¹H-NMR (400 MHz, DMSO-d₆): δ [ppm] = 1.25-1.43 (m, 1H), 1.50-1.70 (m, |
| | 1H), 1.79-1.94 (m,2H), 1.98-2.11 (m, 1H), 2.25-2.36 (m, 1H), 3.10-3.24 |
| | (m, 1H), 3.36-3.43 (m, 1H), 4.83 (dd: 1H), 7.15 (t, 1H), 7.45 (ddd, 1H), 7.63- |
| | 7.68 (m, 1H), 7.69-7.76 (m, 2H), 7.90-7.99 (m, 2H), 8.21 (dd, 1H), 8.28 (dd, |
| | 1H), 10.52 (br s, 1H). |
| Example 630 | 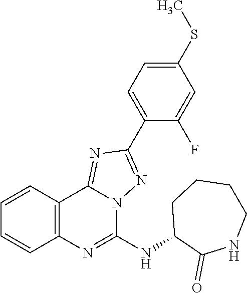 [SMILES: CSc1ccc(-c2nc3c4ccccc4nc(N[C@@H]4CCCCNC4=O)n3n2)c(F)c1] |
| | (3R)-3-({2-[2-fluoro-4-(methylsulfanyl)phenyl][1,2,4]triazolo[1,5- |
| | c]quinazolin-5-yl}amino)azepan-2-one |
| | LC-MS (Method 2): Rₜ = 1.39 min; MS (ESIneg): m/z = 435 [M − H]⁻ |
| | ¹H-NMR (400 MHz, DMSO-d₆): δ [ppm] = 1.21-1.38 (m, 1H), 1.49-1.63 (m, |
| | 1H), 1.81-1.95 (m, 2H), 1.98-2.07 (m, 1H), 2.30-2.38 (m, 1H), 2.59 (s, 3H), |
| | 3.11-3.22 (m, 1H), 4.83 (br dd, 1H), 7.30 (dd, 1H), 7.34 (dd, 1H), 7.46 (ddd, |
| | 1H), 7.66-7.79 (m, 3H), 8.16-8.25 (m, 2H), 8.29 (dd, 1H). |
| Example 631 | 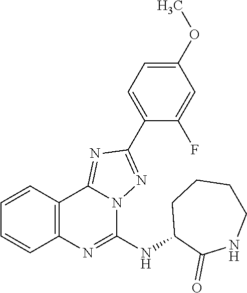 [SMILES: COc1ccc(-c2nc3c4ccccc4nc(N[C@@H]4CCCCNC4=O)n3n2)c(F)c1] |
| | (3R)-3-{[2-(2-fluoro-4-methoxyphenyl)[1,2,4]triazolo[1,5-c]quinazolin-5- |
| | yl]amino}azepan-2-one |
| | LC-MS (Method 2): Rₜ = 1.31 min; MS (ESIneg): m/z = 419 [M − H]⁻ |
| | ¹H-NMR (400 MHz, DMSO-d₆): δ [ppm] = 1.26-1.38 (m, 1H), 1.47-1.64 (m, |
| | 1H), 1.80-1.95 (m, 2H), 1.98-2.07 (m, 1H), 2.30-2.37 (m, 1H), 3.12-3.21 |
| | (m, 1H), 3.35-3.42 (m, 1H), 3.88 (s, 3H), 4.83 (dd, 1H), 7.01 (dd, 1H), 7.06 |
| | (dd, 1H), 7.45 (ddd, 1H), 7.65-7.71 (m, 2H), 7.71-7.77 (m, 1H), 8.15 8.25 |
| | (m, 2H), 8.29 (dd, 1H). |
| Example 632 | 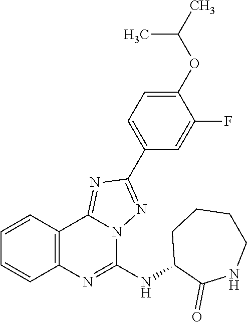 [SMILES: CC(C)Oc1ccc(-c2nc3c4ccccc4nc(N[C@@H]4CCCCNC4=O)n3n2)cc1F] |
| | (3R)-3-[(2-{3-fluoro-4-[(propan-2-yl)oxy]phenyl}[1,2,4]triazolo[1,5- |
| | c]quinazolin-5-yl)amino]azepan-2-one |
| | LC-MS (Method 2): Rₜ = 1.47 min; MS (ESIneg): m/z = 447 [M − H]⁻ |
| | ¹H-NMR (400 MHz, DMSO-d₆): δ [ppm] = 1.18-1.32 (m, 1H), 1.35 (d, 6H), |
| | 1.50-1.62 (m, 1H), 1.81-1.94 (m, 2H), 1.97-2.07 (m, 1H), 2.27-2.35 (m, 1H), |
| | 3.12-3.25 (m, 1H), 4.73-4.86 (m, 2H), 7.39 (t, 1H), 7.45 (ddd, 1H), 7.64- |
| | 7.68 (m, 1H), 7.71-7.76 (m, 2H), 7.98 (dd, 1H), 8.02-8.06 (m, 1H) 8.20 (dd, |
| | 1H), 8.29 (dd, 1H). |
| Example 633 | 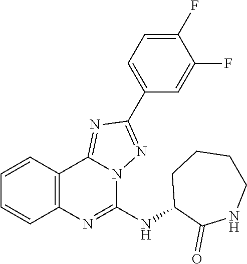 [SMILES: O=C1NCCCC[C@H]1Nc1nc2ccccc2c2nc(-c3ccc(F)c(F)c3)nn12] |
| | (3R)-3-{[2-(3,4-difluorophenyl)[1,2,4]triazolo[1,5-c]quinazolin-5- |
| | yl]amino}azepan-2-one |
| | LC-MS (Method 2): Rₜ = 1.39 min; MS (ESIpos): m/z = 409 [M + H]⁺ |
| | ¹H-NMR (400 MHz, DMSO-d₆): δ [ppm] = 1.26-1.39 (m, 1H), 1.52-1.63 (m, |
| | 1H), 1.81-1.94 (m, 2H), 1.99-2.07 (m, 1H), 2.28-2.35 (m, 1H), 3.12-3.21 |
| | (m, 1H), 4.83 (dd, 1H), 7.46 (ddd, 1H), 7.63-7.71 (m, 2H), 7.72-7.78 (m, 2H), |
| | 8.10-8.15 (m, 1H), 8.16-8.23 (m, 2H), 8.29 (dd, 1H). |
| Example 634 | 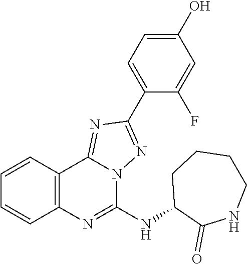 [SMILES: O=C1NCCCC[C@H]1Nc1nc2ccccc2c2nc(-c3ccc(O)cc3F)nn12] |
| | (3R)-3-{[2-(2-fluoro-4-hydroxyphenyl)[1,2,4]triazolo[1,5-c]quinazolin-5- |
| | yl]amino}azepan-2-one |
| | LC-MS (Method 2): Rₜ = 0.74 min; MS (ESIpos): m/z = 407 [M + H]⁺ |
| | ¹H-NMR (400 MHz, DMSO-d₆): δ [ppm] = 1.26-1.38 (m, 1H), 1.49-1.61 (m, |
| | 1H), 1.81-1.95 (m, 2H), 1.98-2.06 (m, 1H), 2.29-2.37 (m, 1H), 3.11-3.21 |
| | (m, 1H), 4.82 (br dd, 1H), 6.76 (dd, 1H), 6.83 (dd, 1H), 7.45 (ddd, 1H), 7.65- |
| | 7.69 (m, 2H), 7.74 (ddd, 1H), 8.09 (t, 1H), 8.22 (dd, 1H), 8.27 (dd, 1H), 10.50 |
| | (br s, 1H). |
| Example 635 | 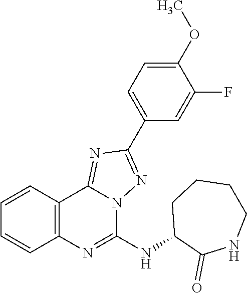 [SMILES: COc1ccc(-c2nc3c4ccccc4nc(N[C@@H]4CCCCNC4=O)n3n2)cc1F] |
| | (3R)-3-{[2-(3-fluoro-4-methoxyphenyl)[1,2,4]triazolo[1,5-c]quinazolin-5- |
| | yl]amino}azepan-2-one |
| | LC-MS (Method 2): Rₜ = 1.34 min; MS (ESIneg): m/z = 419 [M − H]⁻ |
| | ¹H-NMR (400 MHz, DMSO-d₆): δ [ppm] = 1.26-1.40 (m, 1H), 1.47-1.72 (m, |
| | 1H), 1.79-1.95 (m, 2H), 1.97-2.07 (m, 1H), 2.27-2.36 (m, 1H), 3.13-3.32 |
| | (m, 1H), 3.37-3.46 (m, 1H), 3.94 (s, 3H), 4.83 (br dd, 1H), 7.38 (t, 1H), 7.45 |
| | (ddd, 1H), 7.64-7.67 (m, 1H), 7.70-7.76 (m, 2H), 7.99 (dd, 1H), 8.07 (ddd, |
| | 1H), 8.20 (dd, 1H), 8.28 (dd, 1H). |
| Example 636 | 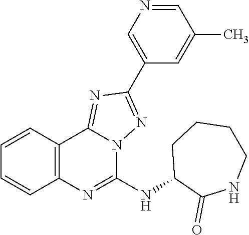 [SMILES: Cc1cncc(-c2nc3c4ccccc4nc(N[C@@H]4CCCCNC4=O)n3n2)c1] |
| | (3R)-3-{[2-(5-methylpyridin-3-yl)[1,2,4]triazolo[1,5-c]quinazolin-5- |
| | yl]amino}azepan-2-one |
| | LC-MS (Method 2): Rₜ = 1.17 min; MS (ESIneg): m/z = 386 [M − H]⁻ |
| | ¹H-NMR (400 MHz, DMSO-d₆): δ [ppm] = 1.23-1.39 (m, 1H), 1.51-1.64 (m, |
| | 1H), 1.80-1.93 (m, 2H), 1.98-2.07 (m, 1H), 2.28-2.38 (m, 1H), 2.45 (s, 3H), |
| | 3.12-3.22 (m, 1H), 3.36-3.42 (m, 1H), 4.84 (br dd, 1H), 7.47 (ddd, 1H), (m, |
| | 1H), 7.64-7.71 (m, 1H), 7.71-7.81 (m, 2H), 8.22 (dd, 1H), 8.31 (dd, 1H), 8.39- |
| | 8.43 (m, 1H), 8.60 (d, 1H), 9.23 (d, 1H). |
| Example 637 | 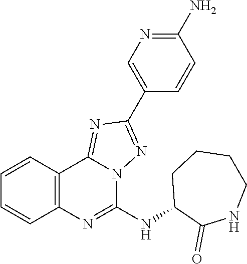 [SMILES: Nc1ccc(-c2nc3c4ccccc4nc(N[C@@H]4CCCCNC4=O)n3n2)cn1] |
| | (3R)-3-{[2-(6-aminopyridin-3-yl)[1,2,4]triazolo[1,5-c]quinazolin-5- |
| | yl]amino}azepan-2-one |
| | LC-MS (Method 2): Rₜ = 1.02 min; MS (ESIpos): m/z = 389 [M + H]⁺ |
| | ¹H-NMR (400 MHz, DMSO-d₆): δ [ppm] = 1.26-1.38 (m, 1H), 1.50-1.61 (m, |
| | 1H), 1.81-1.94 (m, 2H), 1.98-2.06 (m, 1H), 2.28-2.35 (m, 1H), 3.11-3.20 |
| | (m, 1H), 3.34-3.40 (m, 1H), 4.82 (dd, 1H), 6.55 (s, 2H), 6.60 (dd, 1H), 7.43 |
| | (ddd, 1H), 7.64-7.68 (m, 2H), 7.72 (ddd, 1H), 8.17 (dd, 1H), 8.20 (dd, 1H), |
| | 8.27 (dd, 1H), 8.82 (dd, 1H). |
| Example 638 | 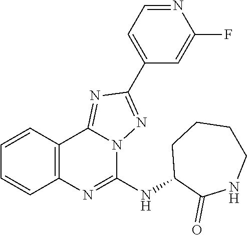 [SMILES: O=C1NCCCC[C@H]1Nc1nc2ccccc2c2nc(-c3ccnc(F)c3)nn12] |
| | (3R)-3-{[2-(2-fluoropyridin-4-yl)[1,2,4]triazolo[1,5-c]quinazolin-5- |
| | yl]amino}azepan-2-one |
| | LC-MS (Method 2): Rₜ = 1.23 min; MS (ESIpos): m/z = 392 [M + H]⁺ |
| Example 639 | 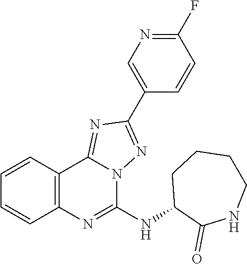 [SMILES: O=C1NCCCC[C@H]1Nc1nc2ccccc2c2nc(-c3ccc(F)nc3)nn12] |
| | (3R)-3-{[2-(6-fluoropyridin-3-yl)[1,2,4]triazolo[1,5-c]quinazolin-5- |
| | yl]amino}azepan-2-one |
| | LC-MS (Method 2): Rₜ = 1.22 min; MS (ESIneg): m/z = 390 [M − H]⁻ |
| | ¹H-NMR (400 MHz, DMSO-d₆): δ [ppm] = 1.25-1.43 (m, 1H), 1.51-1.63 (m, |
| | 1H), 1.81-1.95 (m, 2H), 1.98-2.07 (m, 1H), 2.28-2.35 (m, 1H), 3.12-3.20 |
| | (m, 2H), 4.84 (br dd, 1H), 7.42-7.49 (m, 2H), 7.67-7.70 (m, 1H), 7.74-7.79 |
| | (m, 2H), 8.22 (dd, 1H), 8.31 (dd, 1H), 8.77 (td, 1H), 9.09 (d, 1H). |
| Example 640 | 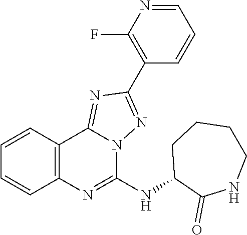 [SMILES: O=C1NCCCC[C@H]1Nc1nc2ccccc2c2nc(-c3cccnc3F)nn12] |
| | (3R)-3-{[2-(2-fluoropyridin-3-yl)[1,2,4]triazolo[1,5-c]quinazolin-5- |
| | yl]amino}azepan-2-one |
| | LC-MS (Method 2): Rₜ = 1.15 min; MS (ESIpos): m/z = 392 [M + H]⁺ |
| | ¹H-NMR (400 MHz, DMSO-d₆): δ [ppm] = 1.26-1.38 (m, 1H), 1.50-1.62 (m, |
| | 1H), 1.81-2.07 (m, 3H), 2.30-2.38 (m, 1H), 3.12-3.21 (m, 1H), 4.83 (br dd, |
| | 1H), 7.44-7.49 (m, 1H), 7.61 (ddd, 1H), 7.66-7.70 (m, 1H), 7.72-7.78 (m, |
| | 2H), 8.24 (br dd, 1H), 8.29 (dd, 1H), 8.45 (dd, 1H), 8.78 (ddd, 1H). |
| Example 641 | 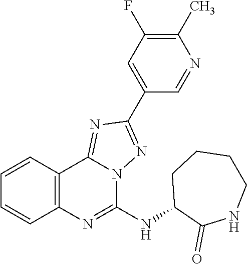 [SMILES: Cc1ncc(-c2nc3c4ccccc4nc(N[C@@H]4CCCCNC4=O)n3n2)cc1F] |
| | (3R)-3-{[2-(5-fluoro-6-methylpyridin-3-yl)[1,2,4]triazolo[1,5-c]quinazolin- |
| | 5-yl]amino}azepan-2-one |
| | LC-MS (Method 2): Rₜ = 1.29 min; MS (ESIpos): m/z = 406 [M + H]⁺ |
| | ¹H-NMR (400 MHz, DMSO-d₆): δ [ppm] = 1.27-1.39 (m, 1H), 1.51-1.63 (m, |
| | 1H), 1.80-1.94 (m, 2H), 1.98-2.09 (m, 1H), 2.27-2.36 (m, 1H), 2.39 (s, 3H), |
| | 3.13-3.21 (m, 1H), 3.36-3.41 (m, 1H), 4.84 (dd, 1H), 7.46 (ddd, 1H), 7.64- |
| | 7.69 (m, 1H), 7.71-7.79 (m, 2H), 8.22 (dd, 1H), 8.29 (dd, 1H), 8.61 (ddd, 1H), |
| | 8.87 (d, 1H). |
| Example 642 | 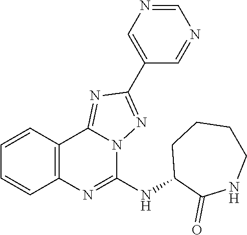 [SMILES: O=C1NCCCC[C@H]1Nc1nc2ccccc2c2nc(-c3cncnc3)nn12] |
| | (3R)-3-{[2-(pyrimidin-5-yl)[1,2,4]triazolo[1,5-c]quinazolin-5- |
| | yl]amino}azepan-2-one |
| | LC-MS (Method 2): Rₜ = 1.06 min; MS (ESIpos): m/z = 375 [M + H]⁺ |
| | ¹H-NMR (400 MHz, DMSO-d₆): δ [ppm] = 1.27-1.39 (m, 1H), 1.54-1.65 (m, |
| | 1H), 1.81-1.92 (m, 2H), 1.98-2.07 (m, 1H), 2.27-2.32 (m, 1H), 3.13-3.22 |
| | (m, 1H), 4.86 (dd, 1H), 7.49 (ddd, 1H), 7.68-7.71 (m, 1H), 7.75-7.82 (m, 2H), |
| | 8.22 (dd, 1H), 8.31-8.36 (m, 1H), 9.39 (s, 1H), 9.57 (s, 2H). |
| Example 643 | 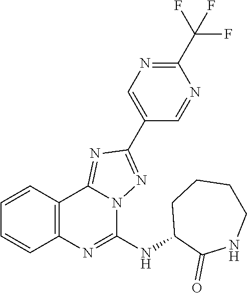 [SMILES: O=C1NCCCC[C@H]1Nc1nc2ccccc2c2nc(-c3cnc(C(F)(F)F)nc3)nn12] |
| | (3R)-3-({2-[2-(trifluoromethyl)pyrimidin-5-yl][1,2,4]triazolo[1,5- |
| | c]quinazolin-5-yl}amino)azepan-2-one |
| | LC-MS (Method 2): Rₜ = 1.34 min; MS (ESIpos): m/z = 443 [M + H]⁺ |
| | ¹H-NMR (400 MHz, DMSO-d₆): δ [ppm] = 1.27-1.40 (m, 1H), 1.54-1.66 (m, |
| | 1H), 1.83-1.95 (m, 2H), 2.00-2.11 (m, 1H), 2.28-2.36 (m, 1H), 3.15-3.25 |
| | (m, 1H), 4.85-4.94 (m, 1H), 7.51 (ddd, 1H), 7.66-7.70 (m, 1H), 7.74-7.80 |
| | (m, 1H), 7.84 (d, 1H), 8.22 (dd, 1H), 8.33 (dd, 1H), 9.80 (s, 2H). |
| Example 644 | 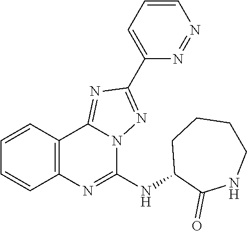 [SMILES: O=C1NCCCC[C@H]1Nc1nc2ccccc2c2nc(-c3cccnn3)nn12] |
| | (3R)-3-{[2-(pyridazin-3-yl)[1,2,4]triazolo[1,5-c]quinazolin-5- |
| | yl]amino}azepan-2-one |
| | LC-MS (Method 2): Rₜ = 1.03 min; MS (ESIpos): m/z = 375 [M + H]⁺ |
| | ¹H-NMR (400 MHz, DMSO-d₆): δ [ppm] = 1.26-1.40 (m, 1H), 1.53-1.65 (m, |
| | 1H), 1.82-1.95 (m, 2H), 1.99-2.08 (m, 1H), 2.28-2.36 (m, 1H), 3.13-3.22 |
| | (m, 1H), 4.86 (br dd, 1H), 7.50 (ddd, 1H), 7.68-7.72 (m, 1H), 7.79 (ddd, 1H), |
| | 7.84 (d, 1H), 8.23 (dd, 1H), 8.34 (dd, 1H), 8.41 (dd, 1H), 9.50 (dd, 1H), 9.97 |
| | (dd, 1H). |

### Example 645

### (3R)-3-{[2-(1,2-thiazol-4-yl)[1,2,4]triazolo[1,5-c]quinazolin-5-yl]amino}azepan-2-one

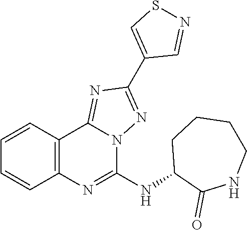 [SMILES: O=C1NCCCC[C@H]1Nc1nc2ccccc2c2nc(-c3cnsc3)nn12]

(3R)-3-[(2-Bromo[1,2,4]triazolo[1,5-c]quinazolin-5-yl)amino]azepan-2-one (100 mg, 267 μmol) was solubilised in DMF (3.3 mL), under argon, 4-(4,4,5,5-tetramethyl-1,3,2-dioxaborolan-2-yl)-1,2-thiazole (113 mg, 533 μmol), XPhos Pd G4 (11.5 mg, 13.3 μmol) and potassium carbonate (400 μL, 2.0 M, 800 μmol) were added and the mixture was stirred for 3 h at 80° C. It was poured into water, filtered, washed with water and the solid was dried under reduced pressure at 45° C. The solid was purified by preparative HPLC to give 40.0 mg (99% purity, 39% yield) of the target compound.

LC-MS (Method 2): Rₜ=1.13 min; MS (ESIpos): m/z=380 [M+H]⁺

¹H-NMR (400 MHz, DMSO-d6): δ [ppm]=1.26-1.39 (m, 1H), 1.50-1.62 (m, 1H), 1.81-1.95 (m, 2H), 1.98-2.07 (m, 1H), 2.27-2.35 (m, 1H), 3.11-3.21 (m, 1H), 3.26-3.42 (m, 1H and water signal), 4.85 (br dd, 1H), 7.47 (ddd, 1H), 7.68 (d, 1H), 7.72 (br d, 1H), 7.76 (ddd, 1H), 8.21 (dd, 1H), 8.30 (dd, 1H), 9.23 (s, 1H), 9.79 (s, 1H).

### Example 646

### (3R)-3-({2-[1-(piperidin-4-yl)-1H-pyrazol-4-yl][1,2,4]triazolo[1,5-c]quinazolin-5-yl}amino)azepan-2-one

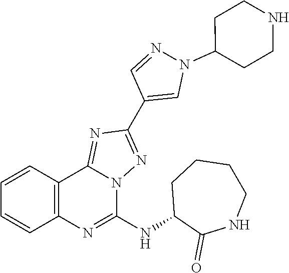 [SMILES: O=C1NCCCC[C@H]1Nc1nc2ccccc2c2nc(-c3cnn(C4CCNCC4)c3)nn12]

tert-Butyl 4-[4-(5-{[(3R)-2-oxoazepan-3-yl]amino}[1,2,4]triazolo[1,5-c]quinazolin-2-yl)-1H-pyrazol-1-yl]piperidine-1-carboxylate (34.6 mg, 63.4 μmol) was solubilised in DCM (3.0 mL), hydrogen chloride (420 μL, 4.0 M in dioxane, 1260 μmol) was added and the mixture was stirred in a pressure tube overnight at 45° C. The mixture was evaporated and the residue was purified by preparative HPLC to give 17.2 mg (95% purity, 58% yield) of the target compound.

LC-MS (Method 2): Rₜ=1.02 min; MS (ESIpos): m/z=446 [M+H]⁺

¹H-NMR (400 MHz, DMSO-d6): δ [ppm]=1.26-1.39 (m, 1H), 1.48-1.61 (m, 1H), 1.80-1.94 (m, 4H), 1.96-2.06 (m, 3H), 2.27-2.35 (m, 1H), 2.55-2.64 (m, 2H), 3.02-3.09 (m, 2H), 3.11-3.21 (m, 1H), 4.32 (tt, 1H), 4.83 (br dd, 1H), 7.44 (ddd, 1H), 7.60 (d, 1H), 7.63-7.68 (m, 1H), 7.70-7.76 (m, 1H), 8.09 (s, 1H), 8.19-8.27 (m, 2H), 8.50 (s, 1H).

### Example 647

### (3R)-3-{[10-(azetidin-3-yl)-2-(4-methoxyphenyl)[1,2,4]triazolo[1,5-c]quinazolin-5-yl]amino}azepan-2-one

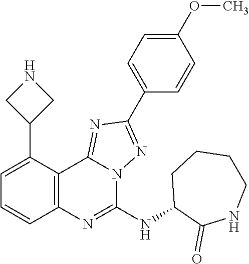 [SMILES: COc1ccc(-c2nc3c4c(C5CNC5)cccc4nc(N[C@@H]4CCCCNC4=O)n3n2)cc1]

tert-Butyl 3-[2-(4-methoxyphenyl)-5-{[(3R)-2-oxoazepan-3-yl]amino}[1,2,4]triazolo[1,5-c]quinazolin-10-yl]azetidine-1-carboxylate (9.10 mg, 16.3 μmol) was solubilised in DCM (500 μL), hydrogen chloride (41 μL, 4.0 M in dioxane, 160 μmol) was added and the mixture was stirred for 2 h at rt. The mixture was basified with TEA (500 μL), filtered and washed with DCM.

LC-MS (Method 2): Rₜ=1.41 min; MS (ESIpos): m/z=458 [M+H]⁺

¹H-NMR (400 MHz, DMSO-d6): δ [ppm]=1.26-1.39 (m, 1H), 1.51-1.64 (m, 1H), 1.80-1.95 (m, 2H), 1.97-2.08 (m, 1H), 2.25-2.35 (m, 1H), 3.12-3.22 (m, 1H), 3.88 (s, 3H), 4.17-4.28 (m, 2H), 4.60 (t, 2H), 4.79-4.87 (m, 1H), 5.26 (quin, 1H), 7.12-7.19 (m, 2H), 7.52 (d, 1H), 7.64 (d, 1H), 7.73-7.80 (m, 2H), 8.22 (dd, 1H), 8.26-8.31 (m, 2H), 8.80 (br s, 1H).

### Example 648

### (3R)-3-[(2-{2-[methanesulfinyl]phenyl}[1,2,4]triazolo[1,5-c]quinazolin-5-yl)amino]azepan-2-one

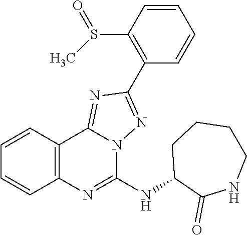 [SMILES: CS(=O)c1ccccc1-c1nc2c3ccccc3nc(N[C@@H]3CCCCNC3=O)n2n1]

(3R)-3-({2-[2-(Methylsulfanyl)phenyl][1,2,4]triazolo[1,5-c]quinazolin-5-yl}amino)azepan-2-one (50.0 mg, 119 μmol) and oxone (45.4 mg, 299 μmol) were solubilised in acetone (5.7 mL) and water (2.3 mL) and the mixture was stirred overnight at rt. The mixture was filtered, washed with water and dried under reduced pressure at 60° C. to give 39.8 mg (100% purity, 77% yield) of the target compound. (mixture of diastereomers)

LC-MS (Method 2): Rₜ=1.15 min; MS (ESIpos): m/z=435 [M+H]⁺

¹H-NMR (400 MHz, DMSO-d6): δ [ppm]=1.26-1.40 (m, 1H), 1.49-1.64 (m, 1H), 1.81-1.96 (m, 2H), 1.99-2.07 (m, 1H), 2.34-2.43 (m, 1H), 3.02 (d, 3H), 3.13-3.23 (m, 1H), 3.34-3.43 (m, 1H), 4.77-4.87 (m, 1H), 7.46-7.52 (m, 1H), 7.68-7.72 (m, 1H), 7.74-7.80 (m, 2H), 7.82 (d, 1H), 7.88 (td, 1H), 8.23 (d, 1H), 8.25-8.30 (m, 1H), 8.33 (dd, 1H), 8.39-8.44 (m, 1H).

### Example 649

### (3R)-3-({2-[2-(methanesulfonyl)phenyl][1,2,4]triazolo[1,5-c]quinazolin-5-yl}amino)azepan-2-one

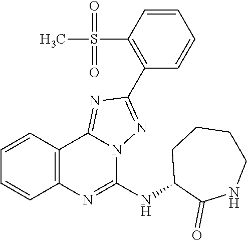 [SMILES: CS(=O)(=O)c1ccccc1-c1nc2c3ccccc3nc(N[C@@H]3CCCCNC3=O)n2n1]

(3R)-3-({2-[2-(Methylsulfanyl)phenyl][1,2,4]triazolo[1,5-c]quinazolin-5-yl}amino)azepan-2-one (50.0 mg, 119 μmol) and 3-chlorobenzenecarboperoxoic acid (66.9 mg, 77% purity, 299 μmol) were stirred in DCM (5.0 mL) for 2 h at rt. The mixture was diluted with disodium sulfurothioate (10%) and extracted three times with DCM. The combined organic layers were dried over a silicone filter and concentrated under reduced pressure. The residue was purified by HPLC to give 31.6 mg (95% purity, 56% yield) of the target compound.

LC-MS (Method 2): Rₜ=1.13 min; MS (ESIpos): m/z=451 [M+H]⁺

¹H-NMR (400 MHz, DMSO-d6): δ [ppm]=1.25-1.38 (m, 1H), 1.50-1.63 (m, 1H), 1.80-1.96 (m, 2H), 1.98-2.07 (m, 1H), 2.30-2.39 (m, 1H), 3.11-3.21 (m, 1H), 3.34-3.41 (m, 1H), 3.71 (s, 3H), 4.80-4.87 (m, 1H), 7.48 (ddd, 1H), 7.70-7.74 (m, 1H), 7.75-7.83 (m, 2H), 7.86-7.95 (m, 3H), 8.17-8.23 (m, 2H), 8.28 (dd, 1H).

### Example 650

### (3R)-3-({2-[2-(S-methylsulfonimidoyl)phenyl][1,2,4]triazolo[1,5-c]quinazolin-5-yl}amino)azepan-2-one

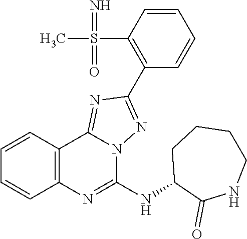 [SMILES: CS(=N)(=O)c1ccccc1-c1nc2c3ccccc3nc(N[C@@H]3CCCCNC3=O)n2n1]

(3R)-3-({2-[2-(Methylsulfanyl)phenyl][1,2,4]triazolo[1,5-c]quinazolin-5-yl}amino)azepan-2-one (100 mg, 239 μmol), bis(acetyoxy)(phenyl)-λ³-iodane (192 mg, 597 μmol) and carbamic acid ammoniate (37.3 mg, 478 μmol) were stirred in methanol (480 μL) overnight at rt under argon. The mixture was evaporated and the residue was purified by preparative HPLC to give 76.7 mg (100% purity, 71% yield) of the target compound. (mixture of diasteromers)

LC-MS (Method 2): Rₜ=1.00 min; MS (ESIpos): m/z=450 [M+H]⁺

¹H-NMR (400 MHz, DMSO-d6): δ [ppm]=1.25-1.38 (m, 1H), 1.48-1.62 (m, 1H), 1.81-1.95 (m, 2H), 1.98-2.08 (m, 1H), 2.31-2.39 (m, 1H), 3.10-3.20 (m, 1H), 3.35-3.42 (m, 1H), 3.53 (d, 3H), 4.12 (s, 1H), 4.80-4.87 (m, 1H), 7.47 (ddd, 1H), 7.69-7.73 (m, 1H), 7.74-7.80 (m, 2H), 7.80-7.85 (m, 3H), 8.18-8.27 (m, 3H).

### Example 651

### (3R)-3-({2-[3-(S-methylsulfonimidoyl)phenyl][1,2,4]triazolo[1,5-c]quinazolin-5-yl}amino)azepan-2-one

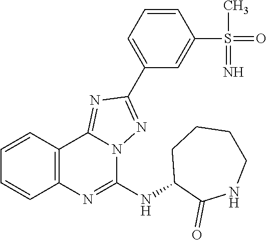 [SMILES: CS(=N)(=O)c1cccc(-c2nc3c4ccccc4nc(N[C@@H]4CCCCNC4=O)n3n2)c1]

(3R)-3-({2-[3-(Methylsulfanyl)phenyl][1,2,4]triazolo[1,5-c]quinazolin-5-yl}amino)azepan-2-one (148 mg, 354 μmol) and carbamic acid ammoniate (55.2 mg, 707 μmol) were solubilised in methanol (740 μL, 18 mmol), bis(acetyoxy)(phenyl)-λ³-iodane (192 mg, 597 μmol) was added and the mixture was stirred at rt under argon overnight. The mixture was concentrated under reduced pressure to give 22.4 mg (14% yield) of the target compound, which was used without further purification. (mixture of diastereomers)

LC-MS (Method 2): Rₜ=1.06 min; MS (ESIpos): m/z=450 [M+H]⁺

¹H-NMR (400 MHz, DMSO-d₆): δ [ppm]=1.53-1.65 (m, 1H), 1.82-1.95 (m, 2H), 1.99-2.08 (m, 1H), 2.29-2.36 (m, 1H), 3.12-3.22 (m, 4H), 4.44 (s, 1H), 4.86 (br dd, 1H), 7.47 (ddd, 1H), 7.67-7.71 (m, 1H), 7.74-7.77 (m, 1H), 7.77-7.81 (m, 1H), 7.85 (t, 1H), 8.12 (dt, 1H), 8.22 (dd, 1H), 8.35 (dd, 1H), 8.55 (dt, 1H), 8.82 (t, 1H).

### Example 652

### methyl 2-(1-methyl-1H-pyrazol-4-yl)-5-{[(3R)-2-oxoazepan-3-yl]amino}[1,2,4]triazolo[1,5-c]quinazoline-7-carboxylate

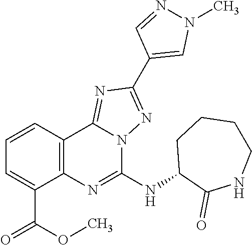 [SMILES: COC(=O)c1cccc2c1nc(N[C@@H]1CCCCNC1=O)n1nc(-c3cnn(C)c3)nc21]

(3R)-3-{[7-Bromo-2-(1-methyl-1H-pyrazol-4-yl)[1,2,4]triazolo[1,5-c]quinazolin-5-yl]amino}azepan-2-one (100 mg, 220 μmol) was suspended in methanol (3.0 mL) and THF (300 μL) in an autoclave (10 mL). 1,1′-Bis(diphenylphosphino)ferrocene-palladium(II)dichloride dichloromethane complex (17.9 mg, 22.0 μmol) and triethylamine (61 μL, 440 μmol) were added. The reaction mixture was purged three times with carbon monoxide at rt. Then, the autoclave was filled with carbon monoxide up to 13.5 bar and it was stirred for 30 min at rt. As the pressure was constant at 13.3 bar the carbon monoxide was released and the autoclave was evacuated under vacuum. The autoclave was filled with carbon monoxide up to 16.3 bar at 20° C. internal temperature. The reaction mixture was stirred for 25 h at 110° C. internal temperature. The reaction mixture was allowed to cool down to rt and the carbon monoxide was removed. The reaction mixture was concentrated and diluted with ethyl acetate. The insoluble residue was filtered off, washed with ethyl acetate and the filtrate was concentrated under reduce pressure to give 115 mg of the title product, which was used without further purification.

LC-MS (Method 2): Rₜ=0.98 min; MS (ESIpos): m/z=435 [M+H]⁺

### Example 653

### propan-2-yl 2-(4-methoxyphenyl)-5-{[(3R)-2-oxoazepan-3-yl]amino}[1,2,4]triazolo[1,5-c]quinazoline-7-carboxylate

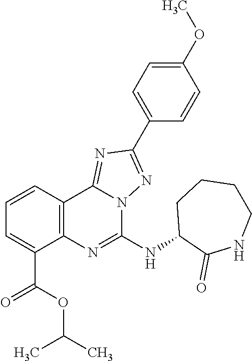 [SMILES: COc1ccc(-c2nc3c4cccc(C(=O)OC(C)C)c4nc(N[C@@H]4CCCCNC4=O)n3n2)cc1]

Methyl 2-(4-methoxyphenyl)-5-{[(3R)-2-oxoazepan-3-yl]amino}[1,2,4]triazolo[1,5-c]quinazoline-7-carboxylate (50.0 mg, 109 μmol) was diluted with THF (4.0 mL), cooled to −78° C., titanium(4+) tetrapropan-2-olate (32 μL, 110 μmol) was added, stirred for 20 min, bromido(ethyl)magnesium (120 μL, 3.0 M in ether, 350 μmol) was added and it was allowed to warm up to rt overnight. It was cooled to −78° C., titanium(4+) tetrapropan-2-olate (96 μL, 330 μmol) was added, stirred for 20 min, bromido(ethyl)magnesium (360 μL, 3.0 M in ether, 1050 μmol) was added and it was allowed to warm up to rt overnight. The mixture was cooled to −78° C., titanium(4+) tetrapropan-2-olate (96 μL, 330 μmol) was added, stirred for 20 min, bromido(ethyl)magnesium (360 μL, 3.0 M in ether, 1050 μmol) was added and the mixture was stirred overnight at 50° C. It was quenched with sat. aqueous ammonium chloride solution, extracted three times with DCM. The organic phase was dried and concentrated under reduced pressure. The residue was purified by preparative HPLC to give 14.5 mg (95% purity, 27% yield) of the target compound.

LC-MS (Method 2): Rₜ=1.38 min; MS (ESIpos): m/z=489 [M+H]⁺

¹H-NMR (500 MHz, DMSO-d₆): δ [ppm]=1.28-1.35 (m, 1H), 1.37 (d, 3H), 1.42 (d, 3H), 1.51-1.61 (m, 1H), 1.77-1.90 (m, 2H), 1.98-2.06 (m, 1H), 2.31-2.38 (m, 1H), 3.17-3.24 (m, 2H), 3.86 (s, 3H), 4.74 (br dd, 1H), 5.22 (spt, 1H), 7.12-7.16 (m, 2H), 7.47 (t, 1H), 7.80 (br d, 1H), 7.85 (dd, 1H), 8.20-8.26 (m, 3H), 8.40 (dd, 1H).

### Example 654

### (3R)-3-{[2-(4-methoxyphenyl)-7-(morpholin-4-yl)[1,2,4]triazolo[1,5-c]quinazolin-5-yl]amino}azepan-2-one

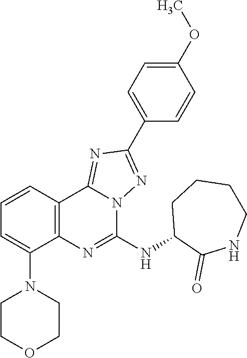 [SMILES: COc1ccc(-c2nc3c4cccc(N5CCOCC5)c4nc(N[C@@H]4CCCCNC4=O)n3n2)cc1]

(3R)-3-{[7-Bromo-2-(4-methoxyphenyl)[1,2,4]triazolo[1,5-c]quinazolin-5-yl]amino}azepan-2-one (100 mg, 208 μmol), morpholine (140 μL, 1.7 mmol), Pd₂(dba)₃ (41.9 mg, 45.7 μmol) and (9,9-dimethyl-9H-xanthene-4,5-diyl)bis(diphenylphosphane) (80.2 mg, 139 μmol) were stirred in 1,4-dioxane (5.0 mL) overnight at 120° C. Morpholine (140 μL, 1.7 mmol), Pd₂(dba)₃ (41.9 mg, 45.7 μmol) and 9,9-dimethyl-9H-xanthene-4,5-diyl)bis(diphenylphosphane (80.2 mg, 139 μmol) were added and the mixture was stirred overnight at 120° C. The mixture was diluted with water and the precipitate was filtered, washed with water and dried under reduced pressure at 60° C. The solid was purified by preparative HPLC to give 10.2 mg (98% purity, 10% yield) of the target compound.

LC-MS (Method 2): Rₜ=1.26 min; MS (ESIpos): m/z=488 [M+H]⁺

1H-NMR (400 MHz, DMSO-d6): δ [ppm]=1.26-1.39 (m, 1H), 1.56-1.69 (m, 1H), 1.74-1.93 (m, 2H), 1.99-2.07 (m, 1H), 2.25 (br d, 1H), 3.14-3.32 (m, 5H), 3.86 (s, 3H), 3.89 (br t, 4H), 4.81 (br dd, 1H), 7.11-7.17 (m, 2H), 7.23 (dd, 1H), 7.34-7.39 (m, 1H), 7.78 (d, 1H), 7.93 (dd, 1H), 8.17 (dd, 1H), 8.20-8.26 (m, 2H).

### Example 655

### (3R)-3-{[2-(4-methoxyphenyl)-10-(morpholin-4-yl)[1,2,4]triazolo[1,5-c]quinazolin-5-yl]amino}azepan-2-one

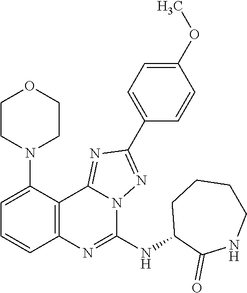 [SMILES: COc1ccc(-c2nc3c4c(N5CCOCC5)cccc4nc(N[C@@H]4CCCCNC4=O)n3n2)cc1]

The reaction was performed using a IKA electrasyn 2.0 system

Solution A: Lithium bromide (2.60 g, 30 mmol) was dissolved in dry N,N-dimethylacetamide (15 mL). Solution B: 1,2-dimethoxyethane-dichloronickel (1:1) (247 mg, 0.80 mmol) and 4,4′-di-tert-butyl-2,2′-bipyridine (215 mg, 0.80 mmol) were dissolved in dry N,N-dimethylacetamide (10 mL).

(3R)-3-{[10-Bromo-2-(4-methoxyphenyl)[1,2,4]triazolo[1,5-c]quinazolin-5-yl]amino}azepan-2-one (50.0 mg, 104 μmol), morpholine (27 μL, 310 μmol), solution A (400 μL), solution B (250 μL) and N,N-dimethylacetamide (1.4 mL) were stirred in a vial. The working electrode (Glassy Carbon) and the counter electrode (Nickel Foam) were inserted into the vial. The mixture was electrolyzed under a constant current of 4 mA for 3 h. The electrodes were rinsed with EtOAc. The reaction mixture was acidified to pH 1 with hydrogen chloride (3M) and extracted with EtOAc. The combined organic layers were dried and concentrated under reduced pressure. The residue was purified by preparative HPLC to give 12.0 mg (100% purity, 24% yield) of the target compound.

¹H-NMR (400 MHz, DMSO-d₆): δ [ppm]=1.26-1.38 (m, 1H), 1.50-1.61 (m, 1H), 1.81-1.94 (m, 2H), 1.98-2.06 (m, 1H), 2.27-2.35 (m, 1H), 2.83-3.28 (m, 5H), 3.86 (s, 3H), 4.00 (br s, 4H), 4.81 (br d, 1H), 7.01 (dd, 1H), 7.16-7.21 (m, 2H), 7.31 (dd, 1H), 7.62 (t, 1H), 7.68 (d, 1H), 8.18-8.26 (m, 3H).

### Example 656

### (6R)-6-{[2-(4-methoxyphenyl)[1,2,4]triazolo[1,5-c]quinazolin-5-yl]amino}-1-methyl-1,4-diazepan-5-one

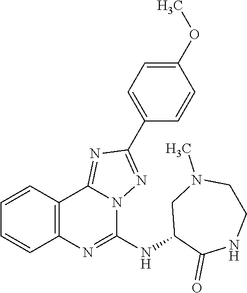 [SMILES: COc1ccc(-c2nc3c4ccccc4nc(N[C@@H]4CN(C)CCNC4=O)n3n2)cc1]

(6R)-6-{[2-(4-Methoxyphenyl)[1,2,4]triazolo[1,5-c]quinazolin-5-yl]amino}-1,4-diazepan-5-one (40.0 mg, 99.1 μmol) was solubilised in butan-2-one (400 μL, 4.5 mmol), potassium carbonate (15.1 mg, 109 μmol) and iodomethane (7.4 μL, 120 μmol) were added and it was stirred for 1 h at rt. Iodomethane (3.1 μL, 50 μmol) and potassium carbonate (6.85 mg, 49.6 μmol) were added and the mixture was stirred for 20 h at rt. It was diluted with water and extracted with DCM. The organic phase was dried and concentrated under reduced pressure. The residue was purified by preparative HPLC to give 2.20 mg (90% purity, 5% yield) of the target compound.

LC-MS (Method 2): Rₜ=1.17 min; MS (ESIpos): m/z=418 [M+H]⁺

¹H-NMR (400 MHz, DMSO-d6): δ [ppm]=2.18 (br t, 1H), 2.30-2.38 (m, 1H), 2.41 (s, 3H), 2.87-2.95 (m, 1H), 3.07-3.16 (m, 1H), 3.24-3.30 (m, 1H and water signal), 3.50-3.59 (m, 1H), 3.86 (s, 3H), 4.99-5.05 (m, 1H), 7.15 (d, 2H), 7.44-7.50 (m, 1H), 7.65-7.71 (m, 2H), 7.72-7.77 (m, 1H), 8.22 (d, 2H), 8.28-8.32 (m, 1H), 8.35 (br dd, 1H).

### Example 657

### ethyl N-[2-(4-methoxyphenyl)[1,2,4]triazolo[1,5-c]quinazolin-5-yl]-D-alaninate

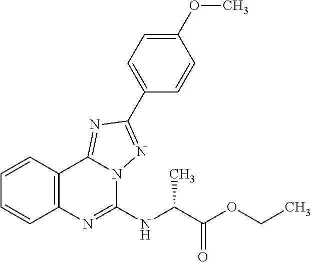 [SMILES: CCOC(=O)[C@@H](C)Nc1nc2ccccc2c2nc(-c3ccc(OC)cc3)nn12]

5-Chloro-2-(4-methoxyphenyl)[1,2,4]triazolo[1,5-c]quinazoline (1.00 g, 93% purity, 3.00 mmol) was solubilised in DMSO (12 mL), N,N-diisopropylethylamine (2.4 mL, 14 mmol) and (2R)-1-ethoxy-1-oxopropan-2-aminium chloride (1.02 g, 95% purity, 6.30 mmol) were added and the mixture was stirred at 60° C. for 1 h. The mixture was poured into water, extracted with butan-1-ol and the organic phase was dried and concentrated under reduced pressure to yield 1.54 g of the target compound, which was used without further purification.

LC-MS (Method 2): Rₜ=1.44 min; MS (ESIpos): m/z=392 [M+H]⁺

### Example 658

### N-[2-(4-methoxyphenyl)[1,2,4]triazolo[1,5-c]quinazolin-5-yl]-D-alanine

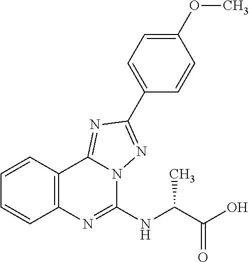 [SMILES: COc1ccc(-c2nc3c4ccccc4nc(N[C@H](C)C(=O)O)n3n2)cc1]

Ethyl N-[2-(4-methoxyphenyl)[1,2,4]triazolo[1,5-c]quinazolin-5-yl]-D-alaninate (300 mg, 766 μmol) was solubilised in methanol (5.0 mL), NaOH (1.5 mL, 2.0 M, 3.1 mmol) was added and stirred for 48 h at rt. The mixture was evaporated, diluted with water, acidified with hydrogen chloride (2.0 M) to pH 4 and the resulting suspension was filtered, washed with water and dried under reduced pressure at 50° C. to yield 177 mg (91% purity, 58% yield) of the target compound, which was used without further purification.

LC-MS (Method 2): Rₜ=0.69 min; MS (ESIpos): m/z=364 [M+H]+

### Example 659

### (3R)-3-({2-[3-(S-methylsulfonimidoyl)phenyl][1,2,4]triazolo[1,5-c]quinazolin-5-yl}amino)azepan-2-one, diastereomer 1

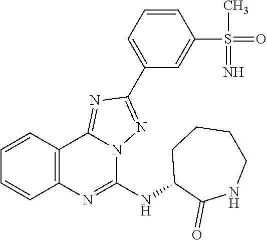 [SMILES: CS(=N)(=O)c1cccc(-c2nc3c4ccccc4nc(N[C@@H]4CCCCNC4=O)n3n2)c1]

The racemate of (3R)-3-({2-[3-(methanesulfonimidoyl)phenyl][1,2,4]triazolo[1,5-c]quinazolin-5-yl}amino)azepan-2-one was separated by chiral HPLC to give 3.50 mg (100% purity, 17% yield) of the target compound.

Instrument: Labomatic HD5000, Labocord-5000; Gilson GX-241, Labcol Vario 4000, column: Cellulose SC 5μ 250×30 mm; eluent A: hexane+0.1 vol-% diethylamine (99%); eluent B: ethanol; isocratic: 50% A+50% B; flowrate 40.0 mL/min; UV 254 nm

Retention time: 4.37 min; α_{D}^{20° C.}: −19° (c=1, DMSO)

Instrument: Agilent HPLC 1260; column: Cellulose SC 3μ 100×4.6 mm; eluent A: hexane+0.1 vol-% diethylamine (99%); Eluent B: ethanol; isocratic: 50% A+50% B; flowrate 1.4 mL/min; temperature: 25° C.; DAD 254 nm

### Example 660

### (3R)-3-({2-[3-(S-methylsulfonimidoyl)phenyl][1,2,4]triazolo[1,5-c]quinazolin-5-yl}amino)azepan-2-one, Diastereomer 2

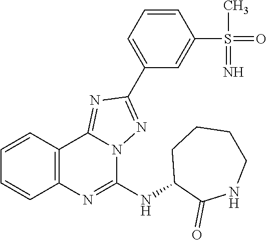 [SMILES: CS(=N)(=O)c1cccc(-c2nc3c4ccccc4nc(N[C@@H]4CCCCNC4=O)n3n2)c1]

The racemate of (3R)-3-({2-[3-(methanesulfonimidoyl)phenyl][1,2,4]triazolo[1,5-c]quinazolin-5-yl}amino)azepan-2-one was separated by chiral HPLC to give 7.70 mg (99% purity, 39% yield) of the target compound.

Instrument: Labomatic HD5000, Labocord-5000; Gilson GX-241, Labcol Vario 4000, column: Cellulose SC 5μ 250×30 mm; eluent A: hexane+0.1 vol-% diethylamine (99%); eluent B: ethanol; isocratic: 50% A+50% B; flowrate 40.0 mL/min; UV 254 nm

Retention time: 5.41 min; α_{D}^{20° C.}: −46° (c=1, in DMSO)

Instrument: Agilent HPLC 1260; column: Cellulose SC 3μ 100×4.6 mm; eluent A: hexane+0.1 vol-% diethylamine (99%); eluent B: ethanol; isocratic: 50% A+50% B; flowrate 1.4 mL/min; temperature: 25° C.; DAD 254 nm

### Example 661

### (3R)-3-({2-[2-(S-methylsulfonimidoyl)phenyl][1,2,4]triazolo[1,5-c]quinazolin-5-yl}amino)azepan-2-one, Diastereomer 1

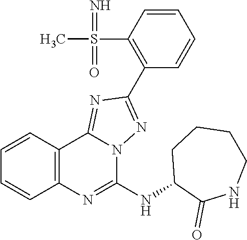 [SMILES: CS(=N)(=O)c1ccccc1-c1nc2c3ccccc3nc(N[C@@H]3CCCCNC3=O)n2n1]

The racemate of (3R)-3-({2-[2-(methanesulfonimidoyl)phenyl][1,2,4]triazolo[1,5-c]quinazolin-5-yl}amino)azepan-2-one was separated by chiral HPLC to give 30.6 mg (100% purity, 42% yield) of the target compound.

Instrument: Labomatic HD5000, Labocord-5000; Gilson GX-241, Labcol Vario 4000, column: Cellulose SC 10μ 250×50 mm; eluent A: ACN+0.1 vol-% diethylamine (99%); eluent B: ethanol; isocratic: 10% B in 22 min; flowrate 100.0 mL/min; UV 254 nm

Retention time: 2.21 min; α_{D}^{20° C.}: −30° (c=1, DMSO)

Instrument: Agilent HPLC 1260; column: Cellulose SC 3μ 50×4.6 mm; eluent A: ACN+0.1 vol-% diethylamine (99%); eluent B: ethanol; isocratic: 10% B in 7 min; flowrate 1.4 mL/min; temperature: 25° C.; DAD 254 nm

### Example 662

### (3R)-3-({2-[2-(S-methylsulfonimidoyl)phenyl][1,2,4]triazolo[1,5-c]quinazolin-5-yl}amino)azepan-2-one, Diastereomer 2

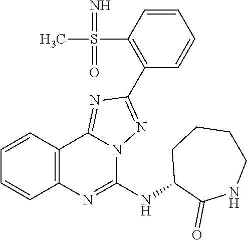 [SMILES: CS(=N)(=O)c1ccccc1-c1nc2c3ccccc3nc(N[C@@H]3CCCCNC3=O)n2n1]

The racemate of (3R)-3-({2-[2-(S-methanesulfonimidoyl)phenyl][1,2,4]triazolo[1,5-c]quinazolin-5-yl}amino)azepan-2-one was separated by chiral HPLC to give 45.2 mg (100% purity, 62% yield) of the target compound.

Instrument: Labomatic HD5000, Labocord-5000; Gilson GX-241, Labcol Vario 4000, column: Cellulose SC 10μ 250×50 mm; eluent A: ACN+0.1 Vol-% diethylamine (99%); eluent B: ethanol; isocratic: 10% B in 22 min; flowrate 100.0 mL/min; UV 254 nm

Retention time: 3.50 min; α_{D}^{20° C.}: −73° (c=1, in DMSO)

Instrument: Agilent HPLC 1260; column: Cellulose SC 3μ 50×4.6 mm; eluent A: ACN+0.1 vol-% diethylamine (99%); eluent B: ethanol; isocratic: 10% B in 7 min; flowrate 1.4 mL/min; temperature: 25° C.; DAD 254 nm

### Example 663

### N-ethyl-2-({2-[2-(trifluoromethoxy)phenyl][1,2,4]triazolo[1,5-c]quinazolin-5-yl}amino)butanamide, Enantiomer 1

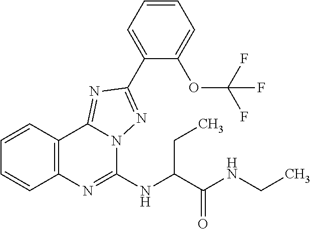 [SMILES: CCNC(=O)C(CC)Nc1nc2ccccc2c2nc(-c3ccccc3OC(F)(F)F)nn12]

The racemate of N-ethyl-2-({2-[2-(trifluoromethoxy)phenyl][1,2,4]triazolo[1,5-c]quinazolin-5-yl}amino)butanamide was separated by chiral HPLC to give 19.6 mg (100% purity, 21% yield) of the target compound.

Instrument: Labomatic HD5000, Labocord-5000; Gilson GX-241, Labcol Vario 4000, column: Cellulose SC 5μ 250×50 mm; eluent A: ethanol; eluent B: methanol; isocratic: 50% B in 12 min; flowrate 100.0 mL/min; UV 280 nm

Retention time: 1.28 min; α_{D}^{20° C.}: +71° (c=1, in DMSO)

Instrument: Agilent HPLC 1260; column: Cellulose SC 3μ 100×4.6 mm; Eluent A: Ethanol+0.1 Vol-% Diethylamine (99%); Eluent B: Methanol; Isocratic: 50% B; flowrate 1.4 mL/min; Temperature: 25° C.; DAD 254 nm

### Example 664

### N-ethyl-2-({2-[2-(trifluoromethoxy)phenyl][1,2,4]triazolo[1,5-c]quinazolin-5-yl}amino)butanamide, Enantiomer 2

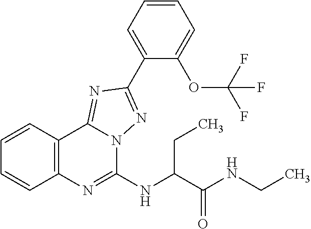 [SMILES: CCNC(=O)C(CC)Nc1nc2ccccc2c2nc(-c3ccccc3OC(F)(F)F)nn12]

The racemate of N-ethyl-2-({2-[2-(trifluoromethoxy)phenyl][1,2,4]triazolo[1,5-c]quinazolin-5-yl}amino)butanamide was separated by chiral HPLC to give 15.0 mg (100% purity, 16% yield) of the target compound.

Instrument: Labomatic HD5000, Labocord-5000; Gilson GX-241, Labcol Vario 4000, column: Cellulose SC 5μ 250×50 mm; eluent A: ethanol; eluent B: methanol; isocratic: 50% B in 12 min; flowrate 100.0 mL/min; UV 280 nm

Retention time: 1.03 min; α_{D}^{20° C.}: −62° (c=1, in DMSO)

Instrument: Agilent HPLC 1260; column: Cellulose SC 3μ 100×4.6 mm; eluent A: ethanol+0.1 vol-% diethylamine (99%); eluent B: methanol; isocratic: 50% B; flowrate 1.4 mL/min; temperature: 25° C.; DAD 254 nm

### Example 665

### N-cyclohexyl-N2-[2-(4-methoxyphenyl)[1,2,4]triazolo[1,5-c]quinazolin-5-yl]-D-alaninamide

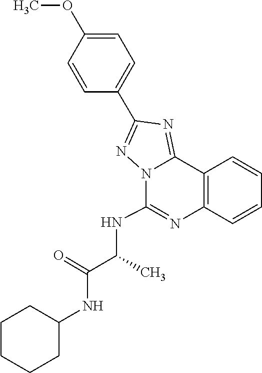 [SMILES: COc1ccc(-c2nc3c4ccccc4nc(N[C@H](C)C(=O)NC4CCCCC4)n3n2)cc1]

Boc-D-Alanine (600 μmol) was dissolved in 1 mL of 1-methyl-2-pyrrolidone and cyclohexanamine (119 mg, 1.20 mmol) in 1.8 mL of 1-methyl-2-pyrrolidone was added, N,N-diisopropylethylamine (233 mg, 1.80 mmol) and 2-(7-aza-1H-benzotriazole-1-yl)-1,1,3,3-tetramethyluronium hexafluorophosphate (456 mg, 1.20 mmol) in 1 mL of 1-methyl-2-pyrrolidone were added. The reaction mixture was placed on a shaker for 1 day and dried by a Christ centrifuge. 2 mL of trifluoracetic acid and 2 mL of dichlormethane were added and the reaction mixture was placed on a shaker for 1 day. The MTPs were dried by a Christ centrifuge. 2 mL of dimethylsulfoxide were added and the reaction mixture was transferred into a metal block with 48 glass vials. 5-Chloro-2-(4-methoxyphenyl)[1,2,4]triazolo[1,5-c]quinazoline (46.6 mg, 150 μmol) in 0.5 mL of dimethylsulfoxide and N,N-diisopropylethylamine (116 mg, 900 μmol) was added and the reaction mixture was heated for 4 h at 70° C. The crude mixture was filtered through a pad of Celite and purified by preparative HPLC to give 4.4 mg (80% purity, 5% yield)

LC-MS (Method 5): Rₜ=1.41 min; MS (ESIpos): m/z=445 [M+H]+

The following example was prepared in analogy to example 665:

| | Structure |
|---|---|
| | IUPAC-Name |
| | LC-MS (method): Retention time; Mass found |
| Example | ¹H-NMR |
| Example 666 | 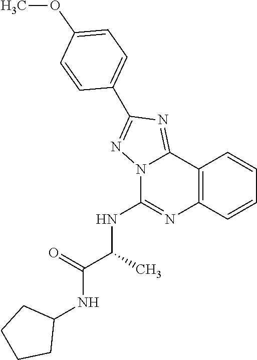 [SMILES: COc1ccc(-c2nc3c4ccccc4nc(N[C@H](C)C(=O)NC4CCCC4)n3n2)cc1] |
| | N-cyclopentyl-N²-[2-(4-methoxyphenyl)[1,2,4]triazolo[1,5-c]quinazolin- |
| | 5-yl]-D-alaninamide |
| | LC-MS (Method 5): Rₜ = 1.35 min; MS (ESIpos): m/z = 431 [M + H]⁺ |

### Example 667

### (3R)-3-{[7-ethoxy-2-(4-methoxyphenyl)[1,2,4]triazolo[1,5-c]quinazolin-5-yl]amino}azepan-2-one

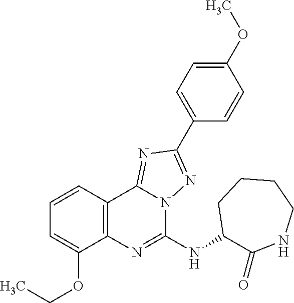

(3R)-3-{[7-bromo-2-(4-methoxyphenyl)[1,2,4]triazolo[1,5-c]quinazolin-5-yl]amino}azepan-2-one (50.0 mg, 104 μmol), tBuBrettPhos Pd G3 (8.88 mg, 10.4 μmol), tBuBrettPhos (5.03 mg, 10.4 μmol;) and cesium carbonate (47.4 mg, 145 μmol) were sealed in a vessel and flushed with Argon. Toluene (1 mL) (pre-flushed with Ar) and the ethanol (59 μl) were added sequentially. The mixture was stirred at 80° C. overnight.

The reaction mixture was cooled to rt, diluted with EtOH and filtered. The filtrate was concentrated and purified by preparative HPLC to give 5 mg (10% yield, 95% purity) of the title compound.

1H-NMR (400 MHz, DMSO-d6): δ [ppm]=1.27-1.39 (m, 2H), 1.46 (t, 3H), 1.49-1.60 (m, 1H), 1.79-1.94 (m, 2H), 1.99-2.12 (m, 1H), 2.42 (br d, 1H), 3.11-3.26 (m, 1H), 3.86 (s, 3H), 4.21 (q, 2H), 4.82 (br dd, 1H), 7.07-7.17 (m, 2H), 7.25-7.31 (m, 1H), 7.32-7.39 (m, 1H), 7.62 (d, 1H), 7.87 (dd, 1H), 8.15-8.26 (m, 3H).

### Example 668

### (3R)-3-{[7-(2,2-difluoropropoxy)-2-(4-methoxyphenyl)[1,2,4]triazolo[1,5-c]quinazolin-5-yl]amino}azepan-2-one

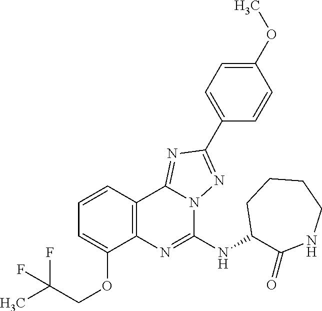

The title compound was prepared similarly to example 667.

### Example 669

### (3R)-3-{[7-hydroxy-2-(4-methoxyphenyl)[1,2,4]triazolo[1,5-c]quinazolin-5-yl]amino}azepan-2-one

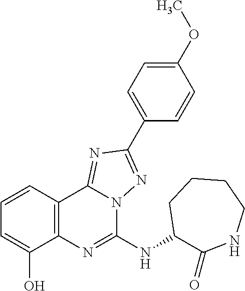

From the previous reaction (example 672) the solid was wash with ethanol and the title compound was obtained in 33% yield (m=14 mg)

1H-NMR (400 MHz, DMSO-d6): δ [ppm]=1.26-1.39 (m, 1H), 1.51-1.66 (m, 1H), 1.79-1.98 (m, 2H), 1.99-2.23 (m, 2H), 3.09-3.23 (m, 1H), 3.44-3.55 (m, 1H), 3.86 (s, 3H), 5.23 (br dd, 1H), 7.09-7.19 (m, 3H), 7.28 (t, 1H), 7.60 (d, 1H), 7.73 (dd, 1H), 8.14 (br dd, 1H), 8.17-8.26 (m, 2H), 9.12 (s, 1H).

### Example 670

### (3R)-3-{[2-(4-methoxyphenyl)-7-(3,3,3-trifluoropropoxy)[1,2,4]triazolo[1,5-c]quinazolin-5-yl]amino}azepan-2-one

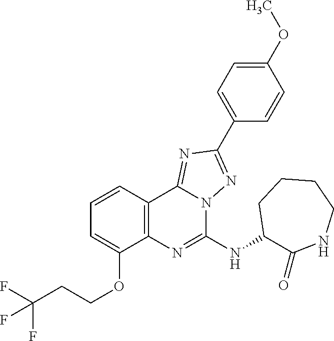

The title compound was prepared similarly to example 667 and obtained in 11% yield (3 mg, 98% purify)

1H-NMR (400 MHz, DMSO-d6): δ [ppm]=1.26-1.41 (m, 2H), 1.48-1.66 (m, 1H), 1.78-1.94 (m, 1H), 2.01 (br dd, 1H), 2.38 (br s, 1H), 2.89 (qt, 2H), 3.10-3.28 (m, 2H), 3.86 (s, 3H), 4.42 (td, 2H), 4.82 (br dd, 1H), 7.04-7.20 (m, 2H), 7.28-7.44 (m, 2H), 7.68 (d, 1H), 7.92 (dd, 1H), 8.13-8.29 (m, 3H).

The following examples were prepared similarly to example 432:

| | Structure |
|---|---|
| | IUPAC-Name |
| | LC-MS (method): Retention time; Mass found |
| Example | ¹H-NMR |
| Example 671 | 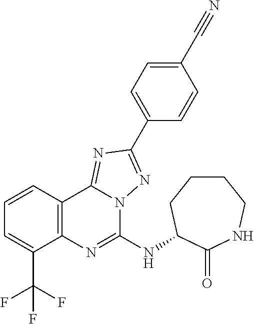 [SMILES: N#Cc1ccc(-c2nc3c4cccc(C(F)(F)F)c4nc(N[C@@H]4CCCCNC4=O)n3n2)cc1] |
| | 4-[5-{[(3R)-2-oxoazepan-3-yl]amino}-7- |
| | (trifluoromethyl)[1,2,4]triazolo[1,5-c]quinazolin-2-yl]benzonitrile |
| | LC-MS (Method 2): Rt = 1.47 min; MS (ESIpos): m/z = 466 [M + H]⁺ |
| | ¹H-NMR (400 MHz, DMSO-d₆): δ [ppm] = 1.26-1.39 (m, 1H), 1.49-1.61 (m, |
| | 1H), 1.79-1.91 (m, 2H), 2.03 (br d, 1H), 2.37-2.44 (m, 1H), 3.16-3.31 (m, |
| | 2H), 4.75 (br dd, 1H), 7.57 (t, 1H), 7.94 (br d, 1H), 8.03-8.07 (m, 2H), 8.12 |
| | (br d, 1H), 8.28 (dd, 1H), 8.40-8.44 (m, 2H), 8.55 (dd, 1H). |
| Example 672 | 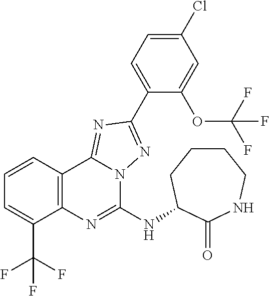 [SMILES: O=C1NCCCC[C@H]1Nc1nc2c(C(F)(F)F)cccc2c2nc(-c3ccc(Cl)cc3OC(F)(F)F)nn12] |
| | (3R)-3-({2-[4-chloro-2-(trifluoromethoxy)phenyl]-7- |
| | (trifluoromethyl)[1,2,4]triazolo[1,5-c]quinazolin-5-yl}amino)azepan-2- |
| | one |
| | LC-MS (Method 1): Rt = 1.69 min; MS (ESIpos): m/z = 557 [M − H]⁺ |
| | ¹H-NMR (400 MHz, DMSO-d₆): δ [ppm] = 1.26-1.38 (m, 1H), 1.46-1.57 (m, |
| | 1H), 1.79-1.92 (m, 2H), 2.03 (br d, 1H), 2.40-2.47 (m, 1H), 3.16-3.31 (m, |
| | 2H), 4.73 (dd, 1H), 7.58 (t, 1H), 7.75-7.81 (m, 2H), 7.92 (d, 1H), 8.13 (dd, |
| | 1H), 8.24 (dd, 1H), 8.43 (d, 1H), 8.53 (dd, 1H). |
| Example 673 | 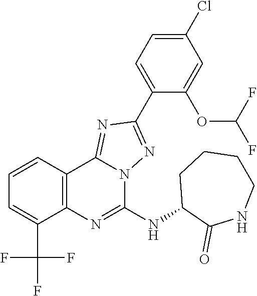 [SMILES: O=C1NCCCC[C@H]1Nc1nc2c(C(F)(F)F)cccc2c2nc(-c3ccc(Cl)cc3OC(F)F)nn12] |
| | (3R)-3-({2-[4-chloro-2-(difluoromethoxy)phenyl]-7- |
| | (trifluoromethyl)[1,2,4]triazolo[1,5-c]quinazolin-5-yl}amino)azepan-2- |
| | one |
| | LC-MS (Method 1): Rt = 1.57 min; MS (ESIpos): m/z = 539 [M − H]⁺ |
| | ¹H-NMR (400 MHz, DMSO-d₆): δ [ppm] = 1.26-1.40 (m, 1H), 1.46-1.59 (m, |
| | 1H), 1.80-1.92 (m, 2H), 1.96-2.08 (m, 1H), 2.40-2.47 (m, 1H), 3.16-3.31 |
| | (m, 2H), 4.75 (dd, 1H), 7.37 (t, 1H), 7.53-7.63 (m, 3H), 7.91 (d, 1H), 8.13 |
| | (d, 1H), 8.21-8.26 (m, 1H), 8.29 (d, 1H), 8.53 (d, 1H). |
| Example 674 | 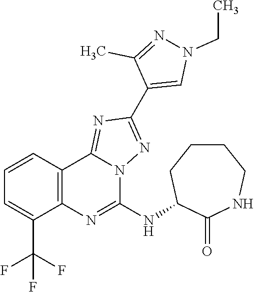 [SMILES: CCn1cc(-c2nc3c4cccc(C(F)(F)F)c4nc(N[C@@H]4CCCCNC4=O)n3n2)c(C)n1] |
| | (3R)-3-{[2-(1-ethyl-3-methyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 1): Rt = 1.37 min; MS (ESIpos): m/z = 473 [M + H]⁺ |
| | ¹H-NMR (400 MHz, DMSO-d₆): δ [ppm] = 1.26-1.38 (m, 1H), 1.42 (t, 3H), |
| | 1.47-1.58 (m, 1H), 1.79-1.91 (m, 2H), 1.97-2.07 (m, 1H), 2.38-2.45 (m, |
| | 1H), 2.59 (s, 3H), 3.15-3.30 (m, 2H), 4.16 (q, 2H), 4.71-4.77 (m, 1H), 7.53 |
| | (t, 1H), 7.80 (d, 1H), 8.09 (dd, 1H), 8.25 (dd, 1H), 8.43 (s, 1H), 8.48 (dd, 1H). |
| Example 675 | 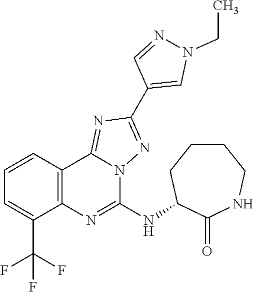 [SMILES: CCn1cc(-c2nc3c4cccc(C(F)(F)F)c4nc(N[C@@H]4CCCCNC4=O)n3n2)cn1] |
| | (3R)-3-{[2-(1-ethyl-1H-pyrazol-4-yl)-7-(trifluoromethyl)[1,2,4]triazolo[1,5- |
| | c]quinazolin-5-yl]amino}azepan-2-one |
| | LC-MS (Method 1): Rt = 1.30 min; MS (ESIpos): m/z = 459 [M + H]⁺ |
| | ¹H-NMR (400 MHz, DMSO-d₆): δ [ppm] = 1.26-1.40 (m, 1H), 1.45 (t, 3H), |
| | 1.49-1.58 (m, 1H), 1.77-1.94 (m, 2H), 1.96-2.08 (m, 1H), 2.36-2.45 (m, |
| | 1H), 3.14-3.31 (m, 2H), 4.25 (q, 2H), 4.72-4.79 (m, 1H), 7.54 (t, 1H), 7.77 |
| | (d, 1H), 8.08-8.12 (m, 2H), 8.27 (dd, 1H), 8.50 (dd, 1H), 8.55 (d, 1H). |
| Example 676 | 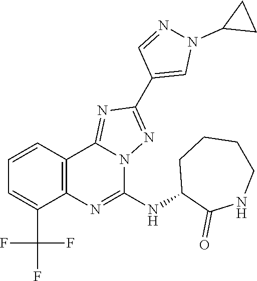 [SMILES: O=C1NCCCC[C@H]1Nc1nc2c(C(F)(F)F)cccc2c2nc(-c3cnn(C4CC4)c3)nn12] |
| | (3R)-3-{[2-(1-cyclopropyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 1): Rt = 1.33 min; MS (ESIpos): m/z = 469 [M − H]⁺ |
| | ¹H-NMR (400 MHz, DMSO-d₆): δ [ppm] = 0.99-1.05 (m, 2H), 1.15-1.20 (m, |
| | 2H), 1.26-1.37 (m, 1H), 1.47-1.57 (m, 1H), 1.79-1.91 (m, 2H), 1.98-2.06 |
| | (m, 1H), 2.36-2.44 (m, 1H), 3.15-3.31 (m, 2H), 3.89 (tt, 1H), 4.72-4.78 |
| | (m, 1H), 7.54 (t, 1H), 7.76 (d, 1H), 8.07-8.12 (m, 2H), 8.26 (dd, 1H), 8.49 |
| | (dd, 1H), 8.57 (s, 1H). |
| Example 677 | 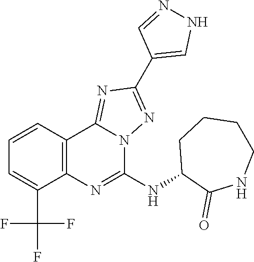 [SMILES: O=C1NCCCC[C@H]1Nc1nc2c(C(F)(F)F)cccc2c2nc(-c3cn[nH]c3)nn12] |
| | (3R)-3-{[2-(1H-pyrazol-4-yl)-7-(trifluoromethyl)[1,2,4]triazolo[1,5- |
| | c]quinazolin-5-yl]amino}azepan-2-one |
| | LC-MS (Method 1): Rt = 1.17 min; MS (ESIpos): m/z = 431 [M + H]⁺ |
| | ¹H-NMR (400 MHz, DMSO-d₆): δ [ppm] = 1.25-1.39 (m, 1H), 1.47-1.59 (m, |
| | 1H), 1.79-1.91 (m, 2H), 1.97-2.07 (m, 1H), 2.39 (br d, 1H), 3.15-3.30 (m, |
| | 2H), 4.76 (dd, 1H), 7.54 (t, 1H), 7.78 (d, 1H), 8.09 (dd, 1H), 8.26 (dd, 2H), |
| | 8.32 (br s, 1H), 8.51 (dd, 1H), 13.36 (br s, 1H). |
| Example 678 | 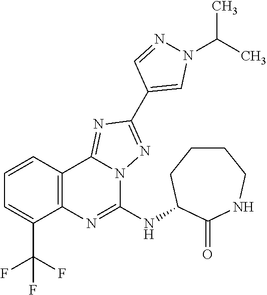 [SMILES: CC(C)n1cc(-c2nc3c4cccc(C(F)(F)F)c4nc(N[C@@H]4CCCCNC4=O)n3n2)cn1] |
| | (3R)-3-({2-[1-(propan-2-yl)-1H-pyrazol-4-yl]-7- |
| | (trifluoromethyl)[1,2,4]triazolo[1,5-c]quinazolin-5-yl}amino)azepan-2- |
| | one |
| | LC-MS (Method 1): Rt = 1.37 min; MS (ESIpos): m/z = 473 [M + H]⁺ |
| | ¹H-NMR (400 MHz, DMSO-d₆): δ [ppm] = 1.25-1.41 (m, 1H), 1.46-1.58 (m, |
| | 1H), 1.50 (d, 6H), 1.79-1.91 (m, 2H), 1.98-2.06 (m, 1H), 2.35-2.45 (m, |
| | 1H), 3.15-3.30 (m, 2H), 4.64 (spt, 1H), 4.76 (br dd, 1H), 7.54 (t, 1H), 7.77 |
| | (d, 1H), 8.08-8.12 (m, 2H), 8.27 (dd, 1H), 8.50 (dd, 1H), 8.55 (s, 1H). |
| Example 679 | 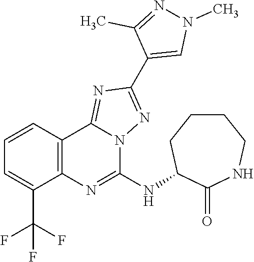 [SMILES: Cc1nn(C)cc1-c1nc2c3cccc(C(F)(F)F)c3nc(N[C@@H]3CCCCNC3=O)n2n1] |
| | (3R)-3-{[2-(1,3-dimethyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 1): Rt = 1.31 min; MS (ESIpos): m/z = 459 [M + H]⁺ |
| | ¹H-NMR (400 MHz, DMSO-d₆): δ [ppm] = 1.24-1.39 (m, 1H), 1.46-1.58 (m, |
| | 1H), 1.79-1.91 (m, 2H), 1.97-2.06 (m, 1H), 2.38-2.47 (m, 1H), 2.58 (s, |
| | 3H), 3.15-3.31 (m, 2H), 3.87 (s, 3H), 4.73 (dd, 1H), 7.53 (t, 1H), 7.80 (d, |
| | 1H), 8.09 (dd, 1H), 8.24 (dd, 1H), 8.39 (s, 1H), 8.48 (dd, 1H). |
| Example 680 | 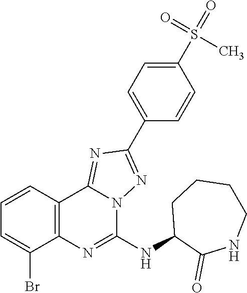 [SMILES: CS(=O)(=O)c1ccc(-c2nc3c4cccc(Br)c4nc(N[C@H]4CCCCNC4=O)n3n2)cc1] |
| | (3S)-3-({7-bromo-2-[4-(methanesulfonyl)phenyl][1,2,4]triazolo[1,5- |
| | c]quinazolin-5-yl}amino)azepan-2-one |
| | LC-MS (method 2): Rₜ = 1.34 min; MS (ESIpos): m/z = 529 [M]⁺ |
| | ¹H-NMR (400 MHz, DMSO-d₆): δ [ppm] = 1.28-1.43 (m, 1H), 1.50-1.63 (m, |
| | 1H), 1.83-1.98 (m, 2H), 2.03-2.11 (m, 1H), 2.41-2.48 (m, 1H), 3.21 (br d, |
| | 1H), 3.32 (s, 3H), 4.85 (br dd, 1H), 7.37 (t, 1H), 7.90 (d, 1H), 8.10 (dd, 1H), |
| | 8.14-8.19 (m, 2H), 8.26 (dd, 1H), 8.33 (dd, 1H), 8.50-8.56 (m, 2H). |
| Example 681 | 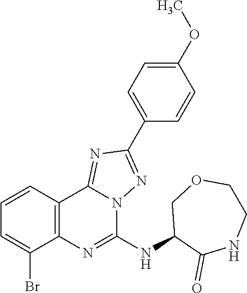 [SMILES: COc1ccc(-c2nc3c4cccc(Br)c4nc(N[C@H]4COCCNC4=O)n3n2)cc1] |
| | (6S)-6-{[7-bromo-2-(4-methoxyphenyl)[1,2,4]triazolo[1,5-c]quinazolin-5- |
| | yl]amino}-1,4-oxazepan-5-one |
| | LC-MS (Method 2): Rt = 1.32 min; MS (ESIpos): m/z = 383 [M + H]⁺ |
| | ¹H-NMR (400 MHz, DMSO-d₆): δ [ppm] = 3.17 (ddd, 1H), 3.39-3.46 (m, 1H), |
| | 3.55-3.64 (m, 2H), 3.86 (s, 3H), 4.00 (dd, 1H), 4.36 (dd, 1H), 5.05 (ddd, |
| | 1H), 7.13-7.18 (m, 2H), 7.36 (t, 1H), 7.84 (d, 1H), 8.08 (dd, 1H), 8.20-8.24 |
| | (m, 2H), 8.31 (dd, 1H), 8.46 (dd, 1H). |
| Example 682 | 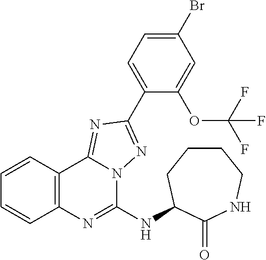 [SMILES: O=C1NCCCC[C@@H]1Nc1nc2ccccc2c2nc(-c3ccc(Br)cc3OC(F)(F)F)nn12] |
| | (3S)-3-({2-[4-bromo-2-(trifluoromethoxy)phenyl][1,2,4]triazolo[1,5- |
| | c]quinazolin-5-yl}amino)azepan-2-one |
| | LC-MS (Method 2): Rt = 1.59 min; MS (ESIpos): m/z = 535 [M + H]⁺ |
| | ¹H-NMR (400 MHz, DMSO-d₆): δ [ppm] = 1.25-1.38 (m, 1H), 1.47-1.59 (m, |
| | 1H), 1.82-1.96 (m, 2H), 1.98-2.07 (m, 1H), 2.31-2.39 (m, 1H), 3.12-3.21 |
| | (m, 1H), 4.81 (br dd, 1H), 7.48 (ddd, 1H), 7.68-7.71 (m, 1H), 7.74-7.79 (m, |
| | 2H), 7.88-7.92 (m, 2H), 8.20 (dd, 1H), 8.28 (dd, 1H), 8.33-8.37 (m, 1H). |
| Example 683 | 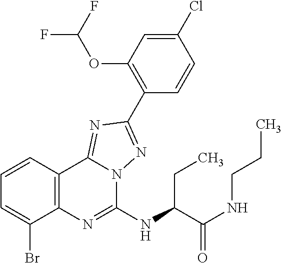 [SMILES: CCCNC(=O)[C@H](CC)Nc1nc2c(Br)cccc2c2nc(-c3ccc(Cl)cc3OC(F)F)nn12] |
| | (2S)-2-({7-bromo-2-[4-chloro-2- |
| | (difluoromethoxy)phenyl][1,2,4]triazolo[1,5-c]quinazolin-5-yl}amino)-N- |
| | propylbutanamide |
| | LC-MS (Method 2): Rt = 1.59 min; MS (ESIpos): m/z = 567 [M + H]⁺ |
| | ¹H-NMR (400 MHz, DMSO-d₆): δ [ppm] = 0.85 (t, 3H), 0.96 (t, 3H), 1.40-1.50 |
| | (m, 2H), 1.89-2.01 (m, 1H), 2.08-2.19 (m, 1H), 3.02-3.18 (m, 2H), 4.70- |
| | 4.76 (m, 1H), 7.36 (t, 1H), 7.39 (t, 1H), 7.56 (d, 1H), 7.62 (dd, 1H), 7.75 (d, |
| | 1H), 8.08 (dd, 1H), 8.26-8.32 (m, 3H). |
| Example 684 | 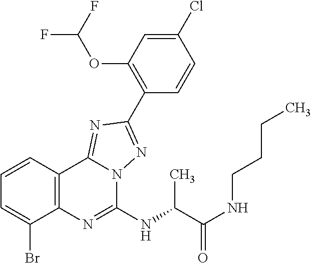 [SMILES: CCCCNC(=O)[C@@H](C)Nc1nc2c(Br)cccc2c2nc(-c3ccc(Cl)cc3OC(F)F)nn12] |
| | N²-{7-bromo-2-[4-chloro-2-(difluoromethoxy)phenyl][1,2,4]triazolo[1,5- |
| | c]quinazolin-5-yl}-N-butyl-D-alaninamide |
| | LC-MS (Method 2): Rt = 1.59 min; MS (ESIpos): m/z = 567 [M + H]⁺ |
| | ¹H-NMR (400 MHz, DMSO-d₆): δ [ppm] = 0.85 (t, 3H), 1.22-1.33 (m, 2H), |
| | 1.37-1.46 (m, 2H), 1.57 (d, 3H), 3.12 (q, 2H), 4.78 (quin, 1H), 7.36 (t, 1H), |
| | 7.40 (t, 1H), 7.56 (d, 1H), 7.62 (dd, 1H), 7.94 (d, 1H), 8.09 (dd, 1H), 8.21 (t, |
| | 1H), 8.25 (d, 1H), 8.29 (dd, 1H). |
| Example 685 | 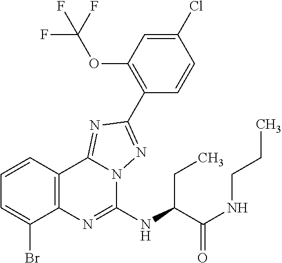 [SMILES: CCCNC(=O)[C@H](CC)Nc1nc2c(Br)cccc2c2nc(-c3ccc(Cl)cc3OC(F)(F)F)nn12] |
| | (2S)-2-({7-bromo-2-[4-chloro-2- |
| | (trifluoromethoxy)phenyl][1,2,4]triazolo[1,5-c]quinazolin-5-yl}amino)-N- |
| | propylbutanamide |
| | LC-MS (Method 2): Rt = 1.68 min; MS (ESIpos): m/z = 585 [M + H]⁺ |
| | ¹H-NMR (400 MHz, DMSO-d₆): δ [ppm] = 0.85 (t, 3H), 0.94 (t, 3H), 1.40-1.51 |
| | (m, 2H), 1.89-2.01 (m, 1H), 2.10-2.22 (m, 1H), 3.02-3.19 (m, 2H), 4.72- |
| | 4.78 (m, 1H), 7.37 (t, 1H), 7.70 (d, 1H), 7.76-7.82 (m, 2H), 8.09 (dd, 1H), |
| | 8.28 (dd, 1H), 8.33 (t, 1H), 8.42 (d, 1H). |
| Example 686 | 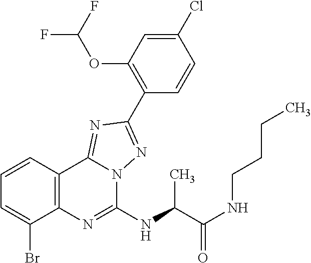 [SMILES: CCCCNC(=O)[C@H](C)Nc1nc2c(Br)cccc2c2nc(-c3ccc(Cl)cc3OC(F)F)nn12] |
| | N²-{7-bromo-2-[4-chloro-2-(difluoromethoxy)phenyl][1,2,4]triazolo[1,5- |
| | c]quinazolin-5-yl}-N-butyl-L-alaninamide |
| | LC-MS (Method 2): Rt = 1.58 min; MS (ESIpos): m/z = 567 [M + H]⁺ |
| | ¹H-NMR (400 MHz, DMSO-d₆): δ [ppm] = 0.85 (t, 3H), 1.22-1.33 (m, 2H), |
| | 1.37-1.46 (m, 2H), 1.57 (d, 3H), 3.12 (q, 2H), 4.74-4.82 (m, 1H), 7.36 (t, |
| | 1H), 7.40 (t, 1H), 7.56 (d, 1H), 7.62 (dd, 1H), 7.94 (br d, 1H), 8.08 (dd, 1H), |
| | 8.21 (t, 1H), 8.25 (d, 1H), 8.28 (dd, 1H). |
| Example 687 | 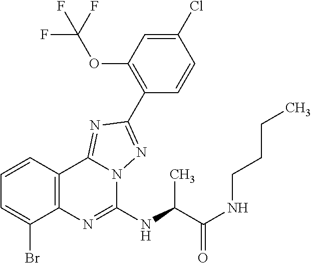 [SMILES: CCCCNC(=O)[C@H](C)Nc1nc2c(Br)cccc2c2nc(-c3ccc(Cl)cc3OC(F)(F)F)nn12] |
| | N²-{7-bromo-2-[4-chloro-2-(trifluoromethoxy)phenyl][1,2,4]triazolo[1,5- |
| | c]quinazolin-5-yl}-N-butyl-L-alaninamide |
| | LC-MS (Method 2): Rt = 1.68 min; MS (ESIpos): m/z = 585 [M + H]⁺ |
| | ¹H-NMR (400 MHz, DMSO-d₆): δ [ppm] = 0.85 (t, 3H), 1.22-1.33 (m, 2H), |
| | 1.38-1.46 (m, 2H), 3.08-3.18 (m, 2H), 4.73-4.81 (m, 1H), 7.36 (t, 1H), |
| | 7.76-7.82 (m, 2H), 7.90 (br d, 1H), 8.09 (dd, 1H), 8.22-8.29 (m, 2H), 8.40 |
| | (d, 1H). |
| Example 688 | 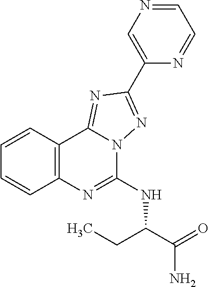 [SMILES: CC[C@H](Nc1nc2ccccc2c2nc(-c3cnccn3)nn12)C(N)=O] |
| | (2S)-2-{[2-(pyrazin-2-yl)[1,2,4]triazolo[1,5-c]quinazolin-5- |
| | yl]amino}butanamide |
| | LC-MS (Method 2): Rt = 0.85 min; MS (ESIpos): m/z = 349 [M + H]⁺ |
| | ¹H-NMR (400 MHz, DMSO-d₆): δ [ppm] = 0.96 (t, 3H), 1.89-2.01 (m, 1H), |
| | 2.03-2.15 (m, 1H), 4.67-4.74 (m, 1H), 7.33 (s, 1H), 7.48 (t, 1H), 7.66 (d, |
| | 1H), 7.71 (br s, 1H), 7.73-7.79 (m, 2H), 8.34 (d, 1H), 8.83-8.90 (m, 2H), |
| | 9.55 (d, 1H). |
| Example 689 | 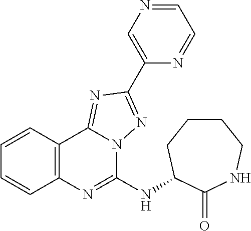 [SMILES: O=C1NCCCC[C@H]1Nc1nc2ccccc2c2nc(-c3cnccn3)nn12] |
| | (3R)-3-{[2-(pyrazin-2-yl)[1,2,4]triazolo[1,5-c]quinazolin-5- |
| | yl]amino}azepan-2-one |
| | LC-MS (Method 2): Rt = 1.05 min; MS (ESIpos): m/z = 375 [M + H]⁺ |
| | ¹H-NMR (400 MHz, DMSO-d₆): δ [ppm] = 1.26-1.39 (m, 1H), 1.51-1.63 (m, |
| | 1H), 1.81-1.96 (m, 2H), 1.98-2.08 (m, 1H), 2.30-2.38 (m, 1H), 3.11-3.22 |
| | (m, 1H), 4.85 (dd, 1H), 7.49 (ddd, 1H), 7.69-7.72 (m, 1H), 7.78 (ddd, 1H), |
| | 7.83 (d, 1H), 8.24 (dd, 1H), 8.35 (dd, 1H), 8.85 (d, 1H), 8.89 (dd, 1H), 9.53 |
| | (d, 1H). |
| Example 690 | 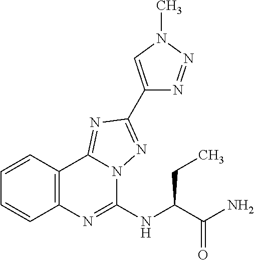 [SMILES: CC[C@H](Nc1nc2ccccc2c2nc(-c3cn(C)nn3)nn12)C(N)=O] |
| | (2S)-2-{[2-(1-methyl-1H-1,2,3-triazol-4-yl)[1,2,4]triazolo[1,5-c]quinazolin- |
| | 5-yl]amino}butanamide |
| | LC-MS (Method 2): Rt = 0.79 min; MS (ESIpos): m/z = 352 [M + H]⁺ |
| | ¹H-NMR (400 MHz, DMSO-d₆): δ [ppm] = 0.95 (t, 3H), 1.87-2.00 (m, 1H), |
| | 2.02-2.14 (m, 1H), 4.18 (s, 3H), 4.67-4.74 (m, 1H), 7.33 (s, 1H), 7.46 (t, |
| | 1H), 7.58 (d, 1H), 7.64 (d, 1H), 7.69-7.78 (m, 2H), 8.24-8.29 (m, 1H), 8.85 |
| | (s, 1H). |
| Example 691 | 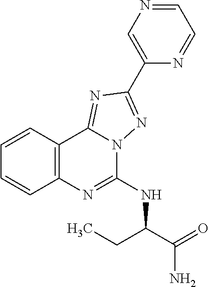 [SMILES: CC[C@@H](Nc1nc2ccccc2c2nc(-c3cnccn3)nn12)C(N)=O] |
| | (2R)-2-{[2-(pyrazin-2-yl)[1,2,4]triazolo[1,5-c]quinazolin-5- |
| | yl]amino}butanamide |
| | LC-MS (Method 2): Rt = 0.84 min; MS (ESIpos): m/z = 349 [M + H]⁺ |
| | ¹H-NMR (400 MHz, DMSO-d₆): δ [ppm] = 0.96 (t, 3H), 1.89-2.01 (m, 1H), |
| | 2.03-2.14 (m, 1H), 4.70 (td, 1H), 7.33 (s, 1H), 7.48 (ddd, 1H), 7.66 (d, 1H), |
| | 7.71 (s, 1H), 7.73-7.79 (m, 2H), 8.34 (dd, 1H), 8.85 (d, 1H), 8.88 (dd, 1H), |
| | 9.55 (d, 1H). |
| Example 692 | 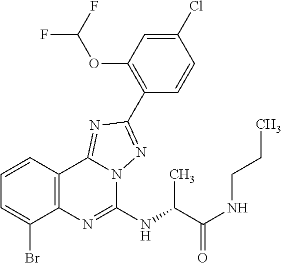 [SMILES: CCCNC(=O)[C@@H](C)Nc1nc2c(Br)cccc2c2nc(-c3ccc(Cl)cc3OC(F)F)nn12] |
| | N²-{7-bromo-2-[4-chloro-2-(difluoromethoxy)phenyl][1,2,4]triazolo[1,5- |
| | c]quinazolin-5-yl}-N-propyl-D-alaninamide |
| | LC-MS (Method 2): Rt = 1.48 min; MS (ESIpos): m/z = 553 [M + H]⁺ |
| | ¹H-NMR (400 MHz, DMSO-d₆): δ [ppm] = 0.84 (t, 3H), 1.39-1.50 (m, 2H), |
| | 1.58 (d, 3H), 3.09 (q, 2H), 4.75-4.83 (m, 1H), 7.36 (t, 1H), 7.40 (t, 1H), 7.56 |
| | (d, 1H), 7.62 (dd, 1H), 7.95 (br s, 1H), 8.08 (dd, 1H), 8.21-8.27 (m, 2H), |
| | 8.28 (dd, 1H). |
| Example 693 | 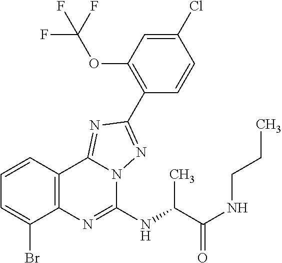 [SMILES: CCCNC(=O)[C@@H](C)Nc1nc2c(Br)cccc2c2nc(-c3ccc(Cl)cc3OC(F)(F)F)nn12] |
| | N²-{7-bromo-2-[4-chloro-2-(trifluoromethoxy)phenyl][1,2,4]triazolo[1,5- |
| | c]quinazolin-5-yl}-N-propyl-D-alaninamide |
| | LC-MS (Method 2): Rt = 1.59 min; MS (ESIpos): m/z = 571 [M + H]⁺ |
| | ¹H-NMR (400 MHz, DMSO-d₆): δ [ppm] = 0.85 (t, 3H), 1.45 (sxt, 2H), 1.58 (d, |
| | 3H), 3.03-3.16 (m, 2H), 4.74-4.83 (m, 1H), 7.36 (t, 1H), 7.76-7.81 (m, |
| | 2H), 7.90 (br s, 1H), 8.09 (dd, 1H), 8.25-8.30 (m, 2H), 8.40 (d, 1H). |
| Example 694 | 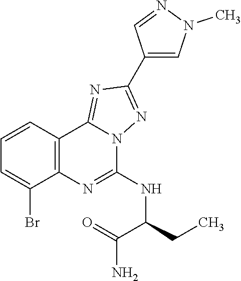 [SMILES: CC[C@H](Nc1nc2c(Br)cccc2c2nc(-c3cnn(C)c3)nn12)C(N)=O] |
| | (2S)-2-{[7-bromo-2-(1-methyl-1H-pyrazol-4-yl)[1,2,4]triazolo[1,5- |
| | c]quinazolin-5-yl]amino}butanamide |
| | LC-MS (Method 2): Rt = 0.97 min; MS (ESIpos): m/z = 429 [M + H]⁺ |
| | ¹H-NMR (400 MHz, DMSO-d₆): δ [ppm] = 0.95 (t, 3H), 1.89-2.01 (m, 1H), |
| | 2.14-2.26 (m, 1H), 3.96 (s, 3H), 4.73 (td, 1H), 7.32 (t, 1H), 7.37 (s, 1H), |
| | 7.69 (d, 1H), 7.76 (s, 1H), 8.05 (dd, 1H), 8.08 (s, 1H), 8.24 (dd, 1H), 8.49 (s, |
| | 1H). |
| Example 695 | 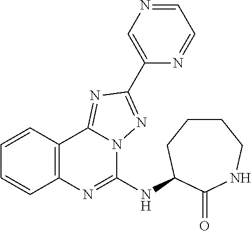 [SMILES: O=C1NCCCC[C@@H]1Nc1nc2ccccc2c2nc(-c3cnccn3)nn12] |
| | (3S)-3-{[2-(pyrazin-2-yl)[1,2,4]triazolo[1,5-c]quinazolin-5- |
| | yl]amino}azepan-2-one |
| | LC-MS (Method 2): Rt = 1.04 min; MS (ESIpos): m/z = 375 [M + H]⁺ |
| | ¹H-NMR (400 MHz, DMSO-d₆): δ [ppm] = 1.27-1.39 (m, 1H), 1.51-1.63 (m, |
| | 1H), 1.81-1.96 (m, 2H), 1.99-2.08 (m, 1H), 2.30-2.38 (m, 1H), 3.12-3.22 |
| | (m, 1H), 4.82-4.88 (m, 1H), 7.49 (ddd, 1H), 7.70 (d, 1H), 7.78 (ddd, 1H), |
| | 7.83 (d, 1H), 8.22-8.26 (m, 1H), 8.34 (dd, 1H), 8.85 (d, 1H), 8.89 (dd, 1H), |
| | 9.53 (d, 1H). |
| Example 696 | 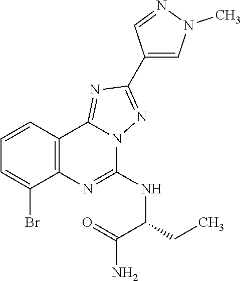 [SMILES: CC[C@@H](Nc1nc2c(Br)cccc2c2nc(-c3cnn(C)c3)nn12)C(N)=O] |
| | (2R)-2-{[7-bromo-2-(1-methyl-1H-pyrazol-4-yl)[1,2,4]triazolo[1,5- |
| | c]quinazolin-5-yl]amino}butanamide |
| | LC-MS (Method 2): Rt = 0.98 min; MS (ESIpos): m/z = 429 [M + H]⁺ |
| | ¹H-NMR (400 MHz, DMSO-d₆): δ [ppm] = 0.75 (t, 3H), 1.71-1.84 (m, 1H), |
| | 2.10-2.22 (m, 1H), 3.98 (s, 3H), 4.63 (q, 1H), 6.67 (d, 1H), 7.29 (t, 1H), 7.32 |
| | (s, 1H), 7.75 (s, 1H), 7.88 (s, 1H), 7.96 (dd, 1H), 8.28 (s, 1H), 8.30 (dd, 1H). |
| Example 697 | 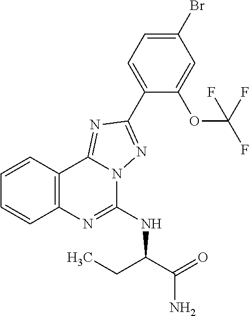 [SMILES: CC[C@@H](Nc1nc2ccccc2c2nc(-c3ccc(Br)cc3OC(F)(F)F)nn12)C(N)=O] |
| | (2R)-2-({2-[4-bromo-2-(trifluoromethoxy)phenyl][1,2,4]triazolo[1,5- |
| | c]quinazolin-5-yl}amino)butanamide |
| | LC-MS (Method 2): Rt = 1.46 min; MS (ESIpos): m/z = 509 [M + H]⁺ |
| | ¹H-NMR (400 MHz, DMSO-d₆): δ [ppm] = 0.93 (t, 3H), 1.85-1.98 (m, 1H), |
| | 2.02-2.13 (m, 1H), 4.69-4.75 (m, 1H), 7.35 (s, 1H), 7.41 (d, 1H), 7.48 |
| | (ddd, 1H), 7.66 (d, 1H), 7.73-7.78 (m, 2H), 7.88-7.93 (m, 2H), 8.28 (dd, |
| | 1H), 8.34 (d, 1H). |
| Example 698 | 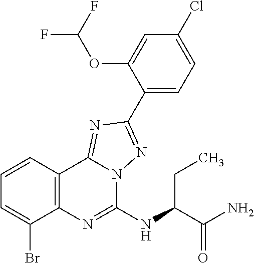 [SMILES: CC[C@H](Nc1nc2c(Br)cccc2c2nc(-c3ccc(Cl)cc3OC(F)F)nn12)C(N)=O] |
| | (2S)-2-({7-bromo-2-[4-chloro-2- |
| | (difluoromethoxy)phenyl][1,2,4]triazolo[1,5-c]quinazolin-5- |
| | yl}amino)butanamide |
| | LC-MS (Method 2): Rt = 1.43 min; MS (ESIpos): m/z = 525 [M + H]⁺ |
| | ¹H-NMR (400 MHz, DMSO-d₆): δ [ppm] = 0.95 (t, 3H), 1.89-2.02 (m, 1H), |
| | 2.15-2.26 (m, 1H), 4.74 (td, 1H), 7.19-7.57 (m, 4H), 7.62 (dd, 1H), 7.73 (d, |
| | 1H), 7.77 (s, 1H), 8.09 (dd, 1H), 8.25-8.30 (m, 2H). |
| Example 699 | 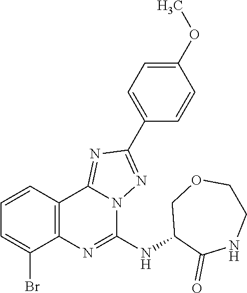 [SMILES: COc1ccc(-c2nc3c4cccc(Br)c4nc(N[C@@H]4COCCNC4=O)n3n2)cc1] |
| | (6R)-6-{[7-bromo-2-(4-methoxyphenyl)[1,2,4]triazolo[1,5-c]quinazolin-5- |
| | yl]amino}-1,4-oxazepan-5-one |
| | LC-MS (Method 2): Rt = 1.32 min; MS (ESIpos): m/z = 383 [M + H]⁺ |
| | ¹H-NMR (400 MHz, DMSO-d₆): δ [ppm] = 3.17 (ddd, 1H), 3.41-3.45 (m, 1H), |
| | 3.55-3.64 (m, 2H), 3.86 (s, 3H), 4.00 (dd, 1H), 4.35 (dd, 1H), 5.02-5.08 |
| | (m, 1H), 7.12-7.17 (m, 2H), 7.36 (t, 1H), 7.84 (d, 1H), 8.08 (dd, 1H), 8.19- |
| | 8.24 (m, 2H), 8.31 (dd, 1H), 8.46 (dd, 1H). |
| Example 700 | 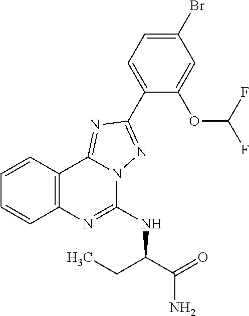 [SMILES: CC[C@@H](Nc1nc2ccccc2c2nc(-c3ccc(Br)cc3OC(F)F)nn12)C(N)=O] |
| | (2R)-2-({2-[4-bromo-2-(difluoromethoxy)phenyl][1,2,4]triazolo[1,5- |
| | c]quinazolin-5-yl}amino)butanamide |
| | LC-MS (Method 2): Rₜ = 1.35 min; MS (ESIpos): m/z = 491 [M + H]⁺ |
| | ¹H-NMR (400 MHz, DMSO-d₆): δ [ppm] = 0.94 (t, 3H), 1.86-1.97 (m, 1H), |
| | 2.00-2.12 (m, 1H), 4.70 (td, 1H), 7.32 (s, 1H), 7.37 (t, 1H), 7.41-7.50 (m, |
| | 2H), 7.64-7.68 (m, 2H), 7.71-7.78 (m, 3H), 8.20 (d, 1H), 8.28 (dd, 1H). |
| Example 701 | 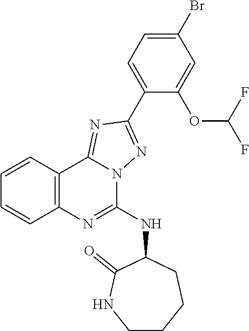 [SMILES: O=C1NCCCC[C@@H]1Nc1nc2ccccc2c2nc(-c3ccc(Br)cc3OC(F)F)nn12] |
| | (3S)-3-({2-[4-bromo-2-(difluoromethoxy)phenyl][1,2,4]triazolo[1,5- |
| | c]quinazolin-5-yl}amino)azepan-2-one |
| | LC-MS (Method 2): Rₜ = 1.48 min; MS (ESIpos): m/z = 517 [M + H]⁺ |
| | ¹H-NMR (400 MHz, DMSO-d₆): δ [ppm] = 1.25-1.38 (m, 1H), 1.48-1.60 (m, |
| | 1H), 1.81-1.95 (m, 2H), 1.98-2.07 (m, 1H), 2.29-2.37 (m, 1H), 3.12-3.21 |
| | (m, 1H), 4.80-4.86 (m, 1H), 7.36 (t, 1H), 7.47 (ddd, 1H), 7.66-7.71 (m, |
| | 2H), 7.72-7.79 (m, 3H), 8.16-8.22 (m, 2H), 8.28 (dd, 1H). |
| Example 702 | 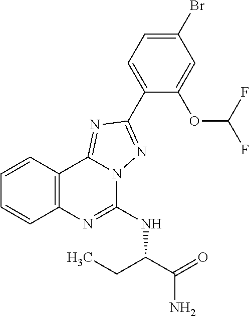 [SMILES: CC[C@H](Nc1nc2ccccc2c2nc(-c3ccc(Br)cc3OC(F)F)nn12)C(N)=O] |
| | (2S)-2-({2-[4-bromo-2-(difluoromethoxy)phenyl][1,2,4]triazolo[1,5- |
| | c]quinazolin-5-yl}amino)butanamide |
| | LC-MS (Method 2): Rₜ = 1.35 min; MS (ESIpos): m/z = 491 [M + H]⁺ |
| | ¹H-NMR (400 MHz, DMSO-d₆): δ [ppm] = 0.95 (t, 3H), 1.85-1.98 (m, 1H), |
| | 2.00-2.12 (m, 1H), 4.70 (td, 1H), 7.32 (s, 1H), 7.37 (t, 1H), 7.42-7.49 (m, |
| | 2H), 7.63-7.68 (m, 2H), 7.71-7.78 (m, 3H), 8.20 (d, 1H), 8.28 (dd, 1H). |
| Example 703 | 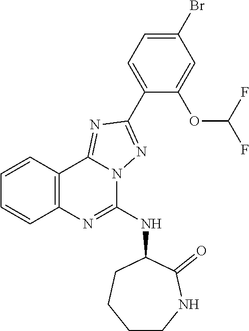 [SMILES: O=C1NCCCC[C@H]1Nc1nc2ccccc2c2nc(-c3ccc(Br)cc3OC(F)F)nn12] |
| | (3R)-3-({2-[4-bromo-2-(difluoromethoxy)phenyl][1,2,4]triazolo[1,5- |
| | c]quinazolin-5-yl}amino)azepan-2-one |
| | LC-MS (Method 2): Rₜ = 1.47 min; MS (ESIpos): m/z = 517 [M + H]⁺ |
| | ¹H-NMR (400 MHz, DMSO-d₆): δ [ppm] = 1.25-1.38 (m, 1H), 1.48-1.60 (m, |
| | 1H), 1.81-1.95 (m, 2H), 1.98-2.07 (m, 1H), 2.29-2.37 (m, 1H), 3.11-3.22 |
| | (m, 1H), 4.83 (br dd, 1H), 7.36 (t, 1H), 7.45-7.50 (m, 1H), 7.65-7.71 (m, |
| | 2H), 7.72-7.79 (m, 3H), 8.16-8.22 (m, 2H), 8.28 (dd, 1H). |
| Example 704 | 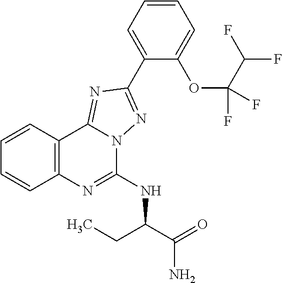 [SMILES: CC[C@@H](Nc1nc2ccccc2c2nc(-c3ccccc3OC(F)(F)C(F)F)nn12)C(N)=O] |
| | (2R)-2-({2-[2-(1,1,2,2-tetrafluoroethoxy)phenyl][1,2,4]triazolo[1,5- |
| | c]quinazolin-5-yl}amino)butanamide |
| | LC-MS (Method 2): Rₜ = 1.31 min; MS (ESIpos): m/z = 463 [M + H]⁺ |
| | ¹H-NMR (400 MHz, DMSO-d₆): δ [ppm] = 0.94 (t, 3H), 1.86-1.98 (m, 1H), |
| | 2.03-2.15 (m, 1H), 4.71 (td, 1H), 6.84 (tt, 1H), 7.40 (s, 1H), 7.43-7.50 (m, |
| | 2H), 7.57 (dd, 1H), 7.61 (td, 1H), 7.64-7.78 (m, 4H), 8.31 (dd, 1H), 8.37 (dd, |
| | 1H). |
| Example 705 | 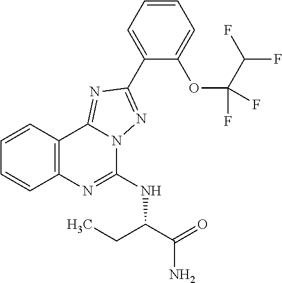 [SMILES: CC[C@H](Nc1nc2ccccc2c2nc(-c3ccccc3OC(F)(F)C(F)F)nn12)C(N)=O] |
| | (2S)-2-({2-[2-(1,1,2,2-tetrafluoroethoxy)phenyl][1,2,4]triazolo[1,5- |
| | c]quinazolin-5-yl}amino)butanamide |
| | LC-MS (Method 2): Rₜ = 1.31 min; MS (ESIpos): m/z = 463 [M + H]⁺ |
| | ¹H-NMR (400 MHz, DMSO-d₆): δ [ppm] = 0.94 (t, 3H), 1.86-1.98 (m, 1H), |
| | 2.03-2.15 (m, 1H), 4.71 (td, 1H), 6.85 (tt, 1H), 7.40 (s, 1H), 7.43-7.49 (m, |
| | 2H), 7.55-7.59 (m, 1H), 7.61 (td, 1H), 7.64-7.72 (m, 2H), 7.73-7.78 (m, |
| | 2H), 8.31 (dd, 1H), 8.37 (dd, 1H). |
| Example 706 | 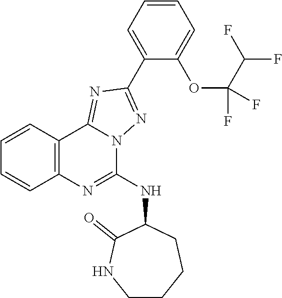 [SMILES: O=C1NCCCC[C@@H]1Nc1nc2ccccc2c2nc(-c3ccccc3OC(F)(F)C(F)F)nn12] |
| | (3S)-3-({2-[2-(1,1,2,2-tetrafluoroethoxy)phenyl][1,2,4]triazolo[1,5- |
| | c]quinazolin-5-yl}amino)azepan-2-one |
| | LC-MS (Method 2): Rₜ = 1.47 min; MS (ESIpos): m/z = 489 [M + H]⁺ |
| | ¹H-NMR (400 MHz, DMSO-d₆): δ [ppm] = 1.26-1.39 (m, 1H), 1.49-1.61 (m, |
| | 1H), 1.81-1.96 (m, 2H), 1.98-2.08 (m, 1H), 2.32-2.40 (m, 1H), 3.12-3.21 |
| | (m, 1H), 4.82 (br dd, 1H), 6.88 (tt, 1H), 7.48 (ddd, 1H), 7.57 (dd, 1H), 7.61 |
| | (td, 1H), 7.67-7.73 (m, 3H), 7.76 (ddd, 1H), 8.29-8.35 (m, 2H), 8.41 (dd, |
| | 1H). |
| Example 707 | 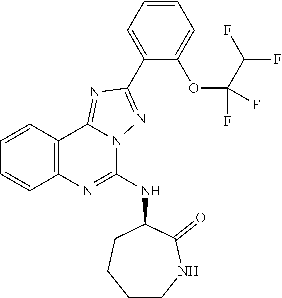 [SMILES: O=C1NCCCC[C@H]1Nc1nc2ccccc2c2nc(-c3ccccc3OC(F)(F)C(F)F)nn12] |
| | (3R)-3-({2-[2-(1,1,2,2-tetrafluoroethoxy)phenyl][1,2,4]triazolo[1,5- |
| | c]quinazolin-5-yl}amino)azepan-2-one |
| | LC-MS (Method 2): Rₜ = 1.47 min; MS (ESIpos): m/z = 489 [M + H]⁺ |
| | ¹H-NMR (400 MHz, DMSO-d₆): δ [ppm] = 1.26-1.39 (m, 1H), 1.49-1.61 (m, |
| | 1H), 1.81-1.96 (m, 2H), 1.99-2.08 (m, 1H), 2.33-2.40 (m, 1H), 3.12-3.21 |
| | (m, 1H), 4.80-4.85 (m, 1H), 6.88 (tt, 1H), 7.48 (ddd, 1H), 7.58 (d, 1H), 7.62 |
| | (td, 1H), 7.67-7.73 (m, 3H), 7.76 (ddd, 1H), 8.30-8.36 (m, 2H), 8.41 (dd, |
| | 1H). |
| Example 708 | 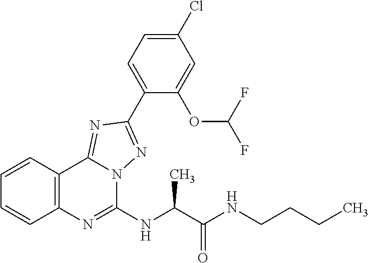 [SMILES: CCCCNC(=O)[C@H](C)Nc1nc2ccccc2c2nc(-c3ccc(Cl)cc3OC(F)F)nn12] |
| | N-butyl-N²-{2-[4-chloro-2-(difluoromethoxy)phenyl][1,2,4]triazolo[1,5- |
| | c]quinazolin-5-yl}-L-alaninamide |
| | LC-MS (Method 2): Rₜ = 1.48 min; MS (ESIpos): m/z = 489 [M + H]⁺ |
| | ¹H-NMR (400 MHz, DMSO-d₆): δ [ppm] = 0.84 (t, 3H), 1.22-1.33 (m, 2H), |
| | 1.34-1.44 (m, 2H), 1.51 (d, 3H), 3.05-3.16 (m, 2H), 4.74 (quin, 1H), 7.39 |
| | (t, 1H), 7.44-7.49 (m, 1H), 7.55 (d, 1H), 7.60-7.66 (m, 3H), 7.75 (ddd, 1H), |
| | 8.18 (t, 1H), 8.25 (d, 1H), 8.28 (dd, 1H). |

The following examples were prepared following the same procedure as for example 408:

| | Structure |
|---|---|
| | IUPAC-Name |
| | LC-MS (method): Retention time; Mass found |
| Example | ¹H-NMR |
| Example 709 | 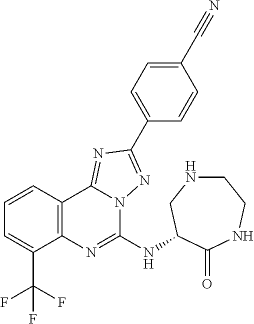 [SMILES: N#Cc1ccc(-c2nc3c4cccc(C(F)(F)F)c4nc(N[C@@H]4CNCCNC4=O)n3n2)cc1] |
| | 4-[5-{[(6R)-5-oxo-1,4-diazepan-6-yl]amino}-7- |
| | (trifluoromethyl)[1,2,4]triazolo[1,5-c]quinazolin-2-yl]benzonitrile |
| | LC-MS (Method 1): Rt = 0.94 min; MS (ESIpos): m/z = 467 [M + H]⁺ |
| | ¹H-NMR (400 MHz, DMSO-d₆): δ [ppm] = 2.52-2.56 (m, 1H, partial in DMSO |
| | signal), 2.74 (dd, 1H), 3.05 (br dd, 1H), 3.09-3.18 (m, 1H), 3.31-3.43 (m, |
| | 2H, partial in water signal), 3.49 (dd, 1H), 4.85 (ddd, 1H), 7.58 (t, 1H), 8.04- |
| | 8.10 (m, 3H), 8.12 (dd, 1H), 8.30 (dd, 1H), 8.42-8.47 (m, 2H), 8.57 (dd, |
| | 1H). |
| Example 710 | 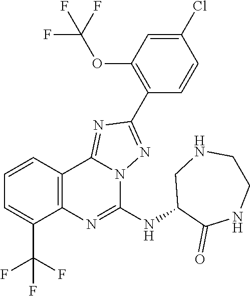 [SMILES: O=C1NCCNC[C@H]1Nc1nc2c(C(F)(F)F)cccc2c2nc(-c3ccc(Cl)cc3OC(F)(F)F)nn12] |
| | (6R)-6-({2-[4-chloro-2-(trifluoromethoxy)phenyl]-7- |
| | (trifluoromethyl)[1,2,4]triazolo[1,5-c]quinazolin-5-yl}amino)-1,4- |
| | diazepan-5-one |
| | LC-MS (Method 1): Rt = 1.13 min; MS (ESIpos): m/z = 560 [M + H]⁺ |
| | ¹H-NMR (400 MHz, DMSO-d₆): δ [ppm] = 2.42-2.55 (m, 1H, partial in DMSO |
| | signal), 2.62-2.72 (m, 2H), 3.01-3.17 (m, 2H), 3.35-3.41 (m, 1H), 3.55 |
| | (dd, 1H), 4.76-4.82 (m, 1H), 7.59 (t, 1H), 7.76-7.82 (m, 2H), 7.95 (d, 1H), |
| | 8.13 (dd, 1H), 8.30 (dd, 1H), 8.42 (d, 1H), 8.54 (dd, 1H). |
| Example 711 | 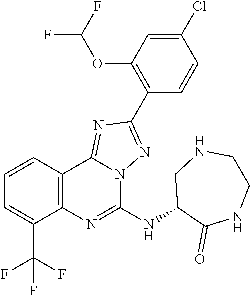 [SMILES: O=C1NCCNC[C@H]1Nc1nc2c(C(F)(F)F)cccc2c2nc(-c3ccc(Cl)cc3OC(F)F)nn12] |
| | (6R)-6-({2-[4-chloro-2-(difluoromethoxy)phenyl]-7- |
| | (trifluoromethyl)[1,2,4]triazolo[1,5-c]quinazolin-5-yl}amino)-1,4- |
| | diazepan-5-one |
| | LC-MS (Method 1): Rt = 1.08 min; MS (ESIpos): m/z = 542 [M + H]⁺ |
| | ¹H-NMR (400 MHz, DMSO-d₆): δ [ppm] = 2.52-2.55 (m, 1H, partial in DMSO |
| | signal), 2.69 (dd, 1H), 3.04 (dd, 1H), 3.08-3.18 (m, 1H), 3.34-3.42 (m, 1H), |
| | 3.51 (dd, 1H), 4.79-4.85 (m, 1H), 7.37 (t, 1H), 7.55-7.63 (m, 3H), 7.96 (br |
| | d, 1H), 8.13 (dd, 1H), 8.25-8.31 (m, 2H), 8.54 (dd, 1H). |
| Example 712 | 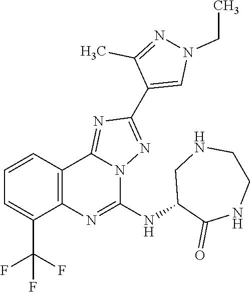 [SMILES: CCn1cc(-c2nc3c4cccc(C(F)(F)F)c4nc(N[C@@H]4CNCCNC4=O)n3n2)c(C)n1] |
| | (6R)-6-{[2-(1-ethyl-3-methyl-1H-pyrazol-4-yl)-7- |
| | (trifluoromethyl)[1,2,4]triazolo[1,5-c]quinazolin-5-yl]amino}-1,4- |
| | diazepan-5-one |
| | LC-MS (Method 1): Rt = 0.83 min; MS (ESIpos): m/z = 474 [M + H]⁺ |
| | ¹H-NMR (400 MHz, DMSO-d₆): δ [ppm] = 1.42 (t, 3H), 2.47-2.55 (m, 1H, |
| | partial in DMSO signal), 2.59 (s, 3H), 2.71 (dd, 1H), 3.05 (dd, 1H), 3.08- |
| | 3.17 (m, 1H), 3.34-3.42 (m, 1H), 3.51 (dd, 1H), 4.17 (q, 2H), 4.79-4.85 (m, |
| | 1H), 7.54 (t, 1H), 7.86 (d, 1H), 8.09 (dd, 1H), 8.29 (dd, 1H), 8.42 (s, 1H), |
| | 8.48 (dd, 1H). |
| Example 713 | 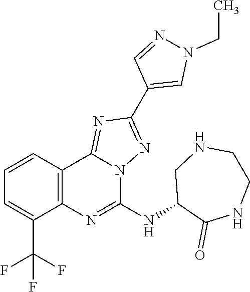 [SMILES: CCn1cc(-c2nc3c4cccc(C(F)(F)F)c4nc(N[C@@H]4CNCCNC4=O)n3n2)cn1] |
| | (6R)-6-{[2-(1-ethyl-1H-pyrazol-4-yl)-7-(trifluoromethyl)[1,2,4]triazolo[1,5- |
| | c]quinazolin-5-yl]amino}-1,4-diazepan-5-one |
| | LC-MS (Method 1): Rt = 0.77 min; MS (ESIpos): m/z = 460 [M + H]⁺ |
| | ¹H-NMR (400 MHz, DMSO-d₆): δ [ppm] = 1.45 (t, 3H), 2.51-2.54 (m, 1H, |
| | partial in DMSO signal), 2.70 (dd, 1H), 3.05 (br dd, 1H), 3.08-3.17 (m, 1H), |
| | 3.34-3.42 (m, 1H), 3.49 (dd, 1H), 4.26 (q, 2H), 4.80-4.86 (m, 1H), 7.54 (t, |
| | 1H), 7.84 (d, 1H), 8.07-8.11 (m, 2H), 8.30 (dd, 1H), 8.50 (dd, 1H), 8.54 (d, |
| | 1H). |
| Example 714 | 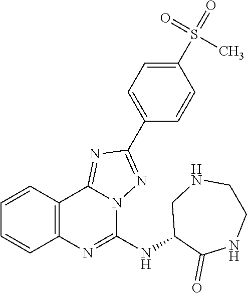 [SMILES: CS(=O)(=O)c1ccc(-c2nc3c4ccccc4nc(N[C@@H]4CNCCNC4=O)n3n2)cc1] |
| | (6R)-6-({2-[4-(methanesulfonyl)phenyl][1,2,4]triazolo[1,5-c]quinazolin-5- |
| | yl}amino)-1,4-diazepan-5-one |
| | LC-MS (method 2): Rₜ = 0.87 min; MS (ESIneg): m/z = 450 [M − H]⁻ |
| | ¹H-NMR (400 MHz, DMSO-d₆): δ [ppm] = 2.67-2.73 (m, 1H), 3.03 (br dd, |
| | 1H), 3.08-3.17 (m, 1H), 3.32 (s, 3H), 3.39-3.45 (m, 2H), 4.87-4.93 (m, |
| | 1H), 7.46-7.51 (m, 1H), 7.69-7.73 (m, 1H), 7.76 (br d, 2H), 8.16 (d, 2H), |
| | 8.29-8.32 (m, 1H), 8.33-8.36 (m, 1H), 8.54 (d, 2H). |
| Example 715 | 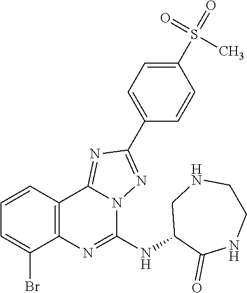 [SMILES: CS(=O)(=O)c1ccc(-c2nc3c4cccc(Br)c4nc(N[C@@H]4CNCCNC4=O)n3n2)cc1] |
| | (6R)-6-({7-bromo-2-[4-(methanesulfonyl)phenyl][1,2,4]triazolo[1,5- |
| | c]quinazolin-5-yl}amino)-1,4-diazepan-5-one |
| | LC-MS (method 2): Rₜ = 1.00 min; MS (ESIpos): m/z = 530 [M + H]⁺ |
| | ¹H-NMR (400 MHz, DMSO-d₆): δ [ppm] = 2.62 (br t, 1H), 2.71-2.77 (m, 1H), |
| | 2.81-2.90 (m, 1H), 3.12 (br d, 1H), 3.15-3.23 (m, 1H), 3.32 (br s, 3H), 3.45- |
| | 3.53 (m, 1H), 3.60 (br d, 1H), 4.98 (br dd, 1H), 7.38 (t, 1H), 8.04 (br d, 1H), |
| | 8.10 (dd, 1H), 8.17 (d, 2H), 8.34 (br dd, 2H), 8.53 (d, 2H). |
| Example 716 | 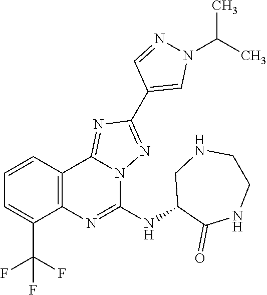 [SMILES: CC(C)n1cc(-c2nc3c4cccc(C(F)(F)F)c4nc(N[C@@H]4CNCCNC4=O)n3n2)cn1] |
| | (6R)-6-({2-[1-(propan-2-yl)-1H-pyrazol-4-yl]-7- |
| | (trifluoromethyl)[1,2,4]triazolo[1,5-c]quinazolin-5-yl}amino)-1,4- |
| | diazepan-5-one |
| | LC-MS (Method 1): Rt = 0.83 min; MS (ESIpos): m/z = 474 [M + H]⁺ |
| | ¹H-NMR (400 MHz, DMSO-d₆): δ [ppm] = 1.50 (d, 6H), 2.51-2.54 (m, 1H, |
| | partial in DMSO signal), 2.69 (dd, 1H), 3.04 (dd, 1H), 3.08-3.16 (m, 1H), |
| | 3.34-3.41 (m, 1H), 3.48 (dd, 1H), 4.64 (spt, 1H), 4.83 (dddd, 1H), 7.55 (t, |
| | 1H), 7.84 (d, 1H), 8.08-8.11 (m, 2H), 8.29 (dd, 1H), 8.50 (dd, 1H), 8.54 (d, |
| | 1H). |
| Example 717 | 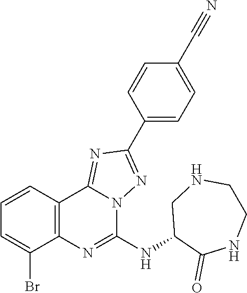 [SMILES: N#Cc1ccc(-c2nc3c4cccc(Br)c4nc(N[C@@H]4CNCCNC4=O)n3n2)cc1] |
| | 4-(7-bromo-5-{[(6R)-5-oxo-1,4-diazepan-6-yl]amino}[1,2,4]triazolo[1,5- |
| | c]quinazolin-2-yl)benzonitrile |
| | LC-MS (Method 2): Rt = 1.12 min; MS (ESIpos): m/z = 477 [M + H]⁺ |
| | ¹H-NMR (400 MHz, DMSO-d₆): δ [ppm] = 2.73 (br dd, 1H), 3.05 (br dd, 1H), |
| | 3.09-3.17 (m, 1H), 3.38-3.47 (m, 1H), 3.51-3.57 (m, 1H), 4.88-4.94 (m, |
| | 1H), 7.37 (t, 1H), 7.93 (d, 1H), 8.05-8.12 (m, 3H), 8.28-8.34 (m, 2H), 8.42- |
| | 8.46 (m, 2H). |
| Example 718 | 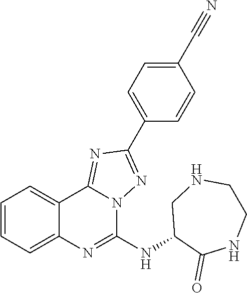 [SMILES: N#Cc1ccc(-c2nc3c4ccccc4nc(N[C@@H]4CNCCNC4=O)n3n2)cc1] |
| | 4-(5-{[(6R)-5-oxo-1,4-diazepan-6-yl]amino}[1,2,4]triazolo[1,5- |
| | c]quinazolin-2-yl)benzonitrile |
| | LC-MS (Method 2): Rt = 1.25 min; MS (ESIpos): m/z = 399 [M + H]⁺ |
| | ¹H-NMR (500 MHz, DMSO-d₆): δ [ppm] = 2.70 (dd, 1H), 3.01-3.06 (m, 1H), |
| | 3.08-3.16 (m, 1H), 3.39-3.47 (m, 2H), 4.87-4.92 (m, 1H), 7.46-7.51 (m, |
| | 1H), 7.70 (d, 1H), 7.74-7.80 (m, 2H), 8.06-8.10 (m, 2H), 8.30-8.34 (m, |
| | 2H), 8.43-8.47 (m, 2H). |
| Example 719 | 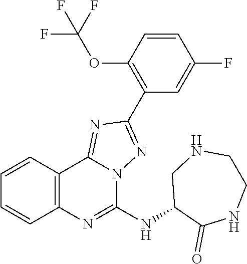 [SMILES: O=C1NCCNC[C@H]1Nc1nc2ccccc2c2nc(-c3cc(F)ccc3OC(F)(F)F)nn12] |
| | (6R)-6-({2-[5-fluoro-2-(trifluoromethoxy)phenyl][1,2,4]triazolo[1,5- |
| | c]quinazolin-5-yl}amino)-1,4-diazepan-5-one |
| | LC-MS (Method 2): Rt = 1.17 min; MS (ESIpos): m/z = 476 [M + H]⁺ |
| | ¹H-NMR (400 MHz, DMSO-d₆): δ [ppm] = 2.60-2.68 (m, 1H), 3.02 (br dd, |
| | 1H), 3.07-3.18 (m, 2H), 3.42-3.47 (m, 2H), 4.83-4.89 (m, 1H), 7.49 (t, |
| | 1H), 7.55-7.62 (m, 1H), 7.67-7.75 (m, 3H), 7.75-7.81 (m, 1H), 8.14 (dd, |
| | 1H), 8.25-8.32 (m, 2H). |
| Example 720 | 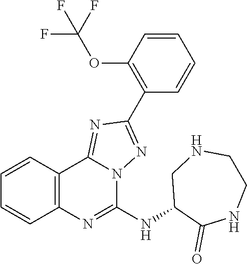 [SMILES: O=C1NCCNC[C@H]1Nc1nc2ccccc2c2nc(-c3ccccc3OC(F)(F)F)nn12] |
| | (6R)-6-({2-[2-(trifluoromethoxy)phenyl][1,2,4]triazolo[1,5-c]quinazolin-5- |
| | yl}amino)-1,4-diazepan-5-one |
| | LC-MS (Method 2): Rt = 1.13 min; MS (ESIpos): m/z = 458 [M + H]⁺ |
| | ¹H-NMR (400 MHz, DMSO-d₆): δ [ppm] = 2.60-2.68 (m, 1H), 2.88-3.06 (m, |
| | 2H), 3.07-3.16 (m, 1H), 3.37-3.48 (m, 2H), 4.86 (ddd, 1H), 7.49 (ddd, 1H), |
| | 7.59-7.67 (m, 2H), 7.70-7.75 (m, 3H), 7.77 (ddd, 1H), 8.25-8.31 (m, 2H), |
| | 8.38 (dd, 1H). |

### Example 721

### 2-(4-methoxyphenyl)-5-{[(3R)-2-oxoazepan-3-yl]amino}[1,2,4]triazolo[1,5-c]quinazoline-7-carbonitrile

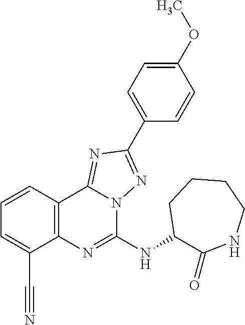

(3R)-3-{[7-Bromo-2-(4-methoxyphenyl)[1,2,4]triazolo[1,5-c]quinazolin-5-yl]amino}azepan-2-one (76.0 mg, 158 μmol), di-mu-chloro[bis(1-phenylprop-2-en-1-yl)]dipalladium (4.38 mg, 7.89 μmol), 1,1′-bis(diphenylphosphanyl)ferrocene (4.38 mg, 7.89 μmol) and zinc cyanide (27.8 mg, 237 μmol) were sealed in a vessel and flushed with argon. N,N-Diisopropylethylamine (55 μL, 320 μmol) and DMA (1 mL) which was first flushed with argon were added. The reaction mixture was stirred at 80° C. overnight. The reaction mixture was diluted with DCM, saturated aqueous sodium hydrogencarbonate solution was added, the layers were separated and the aqueous phase was extracted twice with DCM. The combined organic layers were filtered via a water-repellent filter and concentrated under reduced pressure to yield a crude material that was purified by HPLC to afford 6 mg (9%) of the title compound.

LC-MS (Method 2): Rₜ=1.29 min; MS (ESIpos): m/z=428 [M+H]⁺

¹H-NMR (400 MHz, DMSO-d₆): δ [ppm]=1.28-1.41 (m, 1H), 1.51-1.63 (m, 1H), 1.84-1.98 (m, 2H), 2.00-2.09 (m, 1H), 2.42 (br d, 1H), 3.15-3.24 (m, 1H), 3.86 (s, 3H), 4.84 (br d, 1H), 7.12-7.17 (m, 2H), 7.53 (t, 1H), 7.98 (br d, 1H), 8.19-8.24 (m, 3H), 8.27 (dd, 1H), 8.55 (dd, 1H).

### Example 722

### (3R)-3-({2-[4-methoxy-3-(trifluoromethyl)phenyl][1,2,4]triazolo[1,5-c]quinazolin-5-yl}amino)azepan-2-one

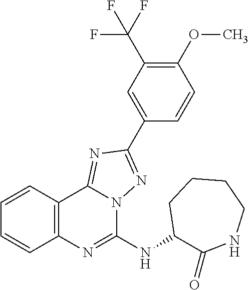

(3R)-3-{[2-(4-Methoxyphenyl)[1,2,4]triazolo[1,5-c]quinazolin-5-yl]amino}azepan-2-one (50.0 mg, 124 μmol), sodium trifluoromethanesulfinate (8.99 mg, 24.8 μmol) and copper(II) trifluoromethanesulfonate (8.99 mg, 24.8 μmol) were sealed in a vessel and flushed with argon. Acetonitrile (1 mL) was purged with argon and added to the reaction vessel. At 0° C. tert-butylhydroperoxide (120 μL, 1.2 mmol) was added dropwise. The reaction mixture was allowed to reach rt and was stirred overnight at rt. The reaction mixture was filtered through a 10 g silica gel column. The column was washed twice with DCM/methanol (9:1) and once with methanol. The filtrate was concentrated under reduced pressure and purified by HPLC yielding 6 mg (10%) of the title compound.

LC-MS (Method 2): Rₜ=1.47 min; MS (ESIneg): m/z=469 [M−H]⁻.

¹H-NMR (400 MHz, ACETONITRILE-d₃): δ [ppm]=1.17-1.29 (m, 1H), 1.36-1.47 (m, 1H), 2.15-2.22 (m, 1H), 3.02-3.21 (m, 2H), 3.78 (s, 3H), 4.66 (ddd, 1H), 6.41-6.47 (m, 1H), 7.14 (d, 1H), 7.23 (ddd, 1H), 7.34 (br d, 1H), 7.45-7.54 (m, 2H), 8.11 (ddd, 1H), 8.29-8.34 (m, 2H).

The following examples were prepared similarly to example 432:

| | Structure |
|---|---|
| | IUPAC-NAME |
| | LC-MS (method): Retention time; Mass found |
| Example | ¹H-NMR |
| Example 723 | 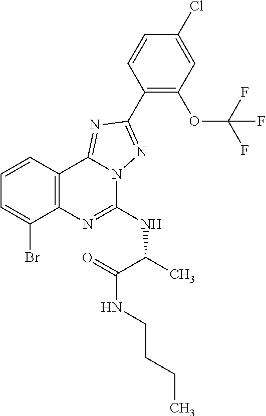 [SMILES: CCCCNC(=O)[C@@H](C)Nc1nc2c(Br)cccc2c2nc(-c3ccc(Cl)cc3OC(F)(F)F)nn12] |
| | N²-{7-bromo-2-[4-chloro-2-(trifluoromethoxy)phenyl][1,2,4]triazolo[1,5- |
| | c]quinazolin-5-yl}-N-butyl-D-alaninamide |
| | LC-MS (Method 2): Rₜ = 1.64 min; MS (ESIpos): m/z = 585 [M + H]⁺ |
| | ¹H-NMR (400 MHz, DMSO-d₆): δ [ppm] = 0.85 (t, 3H), 1.23-1.33 (m, 2H), |
| | 1.38-1.46 (m, 2H), 1.58 (d, 3H), 3.09-3.18 (m, 2H), 4.73-4.82 (m, 1H), |
| | 7.37 (t, 1H), 7.76-7.82 (m, 2H), 7.90 (d, 1H), 8.09 (dd, 1H), 8.24 (t, 1H), |
| | 8.28 (dd, 1H), 8.40 (d, 1H). |
| Example 724 | 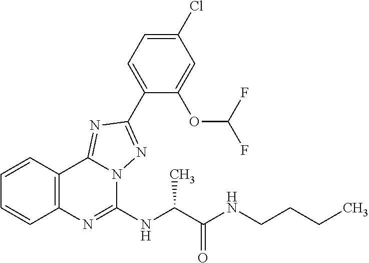 [SMILES: CCCCNC(=O)[C@@H](C)Nc1nc2ccccc2c2nc(-c3ccc(Cl)cc3OC(F)F)nn12] |
| | N-butyl-N²-{2-[4-chloro-2-(difluoromethoxy)phenyl][1,2,4]triazolo[1,5- |
| | c]quinazolin-5-yl}-D-alaninamide |
| | LC-MS (Method 2): Rₜ = 1.45 min; MS (ESIpos): m/z = 489 [M + H]⁺ |
| | ¹H-NMR (400 MHz, DMSO-d₆): δ [ppm] = 0.85 (t, 3H), 1.22-1.33 (m, 2H), |
| | 1.35-1.45 (m, 2H), 1.51 (d, 3H), 3.05-3.17 (m, 2H), 4.75 (quin, 1H), 7.39 (t, |
| | 1H), 7.47 (ddd, 1H), 7.56 (d, 1H), 7.60-7.66 (m, 3H), 7.75 (ddd, 1H), 8.18 (t, |
| | 1H), 8.24-8.30 (m, 2H). |
| Example 725 | 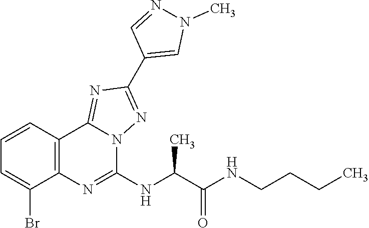 [SMILES: CCCCNC(=O)[C@H](C)Nc1nc2c(Br)cccc2c2nc(-c3cnn(C)c3)nn12] |
| | N²-[7-bromo-2-(1-methyl-1H-pyrazol-4-yl)[1,2,4]triazolo[1,5-c]quinazolin- |
| | 5-yl]-N-butyl-L-alaninamide |
| | LC-MS (Method 2): Rₜ = 1.17 min; MS (ESIpos): m/z = 471 [M + H]⁺ |
| | ¹H-NMR (400 MHz, DMSO-d₆): δ [ppm] = 0.83 (t, 3H), 1.20-1.31 (m, 2H), |
| | 1.34-1.45 (m, 2H), 1.56 (d, 3H), 3.07-3.14 (m, 2H), 3.96 (s, 3H), 4.76 |
| | (quin, 1H), 7.32 (t, 1H), 7.93 (d, 1H), 8.04 (dd, 1H), 8.07 (d, 1H), 8.17 (t, 1H), |
| | 8.24 (dd, 1H), 8.46 (s, 1H). |

### Example 726

### (3R)-3-{[2-(4-methoxyphenyl)-10-(propan-2-yl)[1,2,4]triazolo[1,5-c]quinazolin-5-yl]amino}azepan-2-one

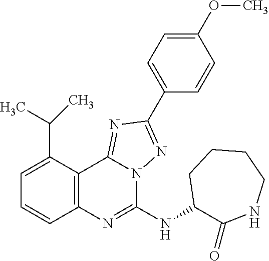

The title compound was prepared similarly to example 393 starting from (3R)-3-{[10-bromo-2-(4-methoxyphenyl)[1,2,4]triazolo[1,5-c]quinazolin-5-yl]amino}azepan-2-one (114 mg, 238 μmol). 39.7 mg (95% purity, 36% yield) of the desired product were obtained after purification by preparative HPLC.

LC-MS (method 2): Rₜ=1.63 min; MS (ESIpos): m/z=445 [M+H]⁺

¹H-NMR (400 MHz, DMSO-d6): δ [ppm]=1.32 (br d, 1H), 1.37 (dd, 6H), 1.50-1.62 (m, 1H), 1.80-1.95 (m, 2H), 1.98-2.06 (m, 1H), 2.27-2.36 (m, 1H), 3.11-3.21 (m, 1H), 3.34-3.42 (m, 1H), 3.86 (s, 3H), 4.82 (dd, 1H), 4.94 (quin, 1H), 7.13-7.19 (m, 2H), 7.41 (dd, 1H), 7.51 (dd, 1H), 7.64-7.71 (m, 2H), 8.17-8.25 (m, 3H).

### Example 727

### (6R)-6-{[7-bromo-2-(4-methoxyphenyl)[1,2,4]triazolo[1,5-c]quinazolin-5-yl]amino}-1,4-diazepan-5-one hydrochloride

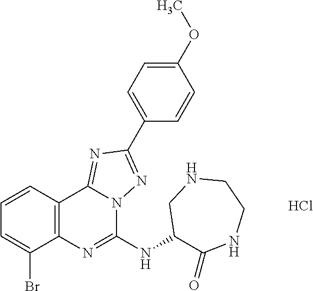

(6R)-6-{[7-bromo-2-(4-methoxyphenyl)[1,2,4]triazolo[1,5-c]quinazolin-5-yl]amino}-1,4-diazepan-5-one (23.7 mg, 49.1 μmol) was stirred in HCl (1.0 mL, 4.0 M in dioxane, 4.0 mmol) for 2 h at RT. The mixture was then concentrated under reduced pressure to give 23.1 mg (90% purity, 82% yield) of the title compound.

LC-MS (method 2): Rₜ=1.18 min; MS (ESIpos): m/z=482 [M+H]⁺

¹H-NMR (400 MHz, DMSO-d6): δ [ppm]=3.06 (br d, 1H), 3.43-3.52 (m, 2H), 3.74-3.83 (m, 1H), 3.84-3.95 (m, 4H), 5.31 (br dd, 1H), 7.17 (d, 2H), 7.38 (t, 1H), 8.09 (dd, 1H), 8.24 (d, 2H), 8.33 (dd, 1H), 8.45 (d, 1H), 8.48 (dd, 1H), 9.42 (br d, 1H), 9.84 (br d, 1H). low purity.

### Example 728

### (+/−)-4,4-dimethyl-3-{[2-(1-methyl-1H-pyrazol-4-yl)-7-(trifluoromethyl)[1,2,4]triazolo[1,5-c]quinazolin-5-yl]amino}pyrrolidin-2-one

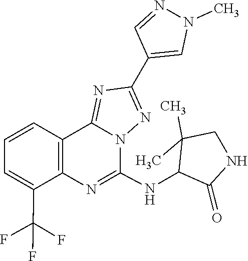 [SMILES: Cn1cc(-c2nc3c4cccc(C(F)(F)F)c4nc(NC4C(=O)NCC4(C)C)n3n2)cn1]

5-Chloro-2-(1-methyl-1H-pyrazol-4-yl)-7-(trifluoromethyl)[1,2,4]triazolo[1,5-c]quinazoline (150 mg, 425 μmol), (+/−)-3-amino-4,4-dimethylpyrrolidin-2-one (81.8 mg, 638 μmol) and N,N-diisopropylethylamine (150 μL, 850 μmol) were stirred in DMSO (3.9 mL) for 2 h at 60° C. The reaction mixture was then cooled to rt and diluted with water. The solid was filtered, washed with water and dried under reduced pressure at 60° C. to give 163 mg (95% purity, 82% yield) of the title product.

LC-MS (Method 1): Rₜ=1.16 min; MS (ESIneg): m/z=443 [M−H]⁻.

¹H-NMR (400 MHz, DMSO-d₆): δ [ppm]=1.05 (s, 3H), 1.28 (s, 3H), 3.05-3.17 (m, 2H), 3.96 (s, 3H), 4.79 (br d, 1H), 7.53 (t, 1H), 8.05-8.10 (m, 3H), 8.15 (d, 1H), 8.47 (s, 1H), 8.50 (dd, 1H).

The following examples were prepared analogously to example 728

| | Structure |
|---|---|
| | IUPAC-NAME |
| | LC-MS (method): Retention time; Mass found |
| Example | ¹H-NMR |
| Example 729 | 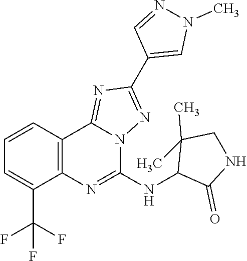 [SMILES: Cn1cc(-c2nc3c4cccc(C(F)(F)F)c4nc(NC4C(=O)NCC4(C)C)n3n2)cn1] |
| | (−)-4,4-dimethyl-3-{[2-(1-methyl-1H-pyrazol-4-yl)-7- |
| | (trifluoromethyl)[1,2,4]triazolo[1,5-c]quinazolin-5-yl]amino}pyrrolidin-2- |
| | one |
| | obtained via chiral HPLC separation of the racemate |
| | LC-MS (Method 1): Rₜ = 1.16 min; MS (ESIneg): m/z = 443 [M − H]⁻ |
| | [α]₅₈₉²⁰ = −116.1 (c = 1.8; DMSO) |
| | ¹H-NMR (400 MHz, DMSO-d₆): δ [ppm] = 1.05 (s, 3H), 1.28 (s, 3H), 3.05- |
| | 3.17 (m, 2H), 3.96 (s, 3H), 4.79 (br d, 1H), 7.53 (t, 1H), 8.05-8.10 (m, 3H), |
| | 8.15 (d, 1H), 8.47 (s, 1H), 8.50 (dd, 1H). |
| Example 730 | 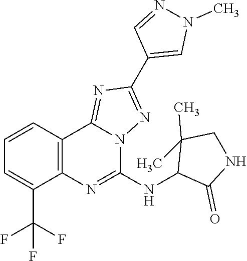 [SMILES: Cn1cc(-c2nc3c4cccc(C(F)(F)F)c4nc(NC4C(=O)NCC4(C)C)n3n2)cn1] |
| | (+)-4,4-dimethyl-3-{[2-(1-methyl-1H-pyrazol-4-yl)-7- |
| | (trifluoromethyl)[1,2,4]triazolo[1,5-c]quinazolin-5-yl]amino}pyrrolidin-2- |
| | one |
| | obtained via chiral HPLC separation of the racemate |
| | LC-MS (Method 1): Rₜ = 1.16 min; MS (ESIneg): m/z = 443 [M − H]⁻ |
| | [α]₅₈₉²⁰ = +113.7 (c = 1.6; DMSO) |
| | ¹H-NMR (400 MHz, DMSO-d₆): δ [ppm] = 1.05 (s, 3H), 1.28 (s, 3H), 3.05- |
| | 3.17 (m, 2H), 3.96 (s, 3H), 4.79 (br d, 1H), 7.53 (t, 1H), 8.05-8.10 (m, 3H), |
| | 8.15 (d, 1H), 8.47 (s, 1H), 8.50 (dd, 1H). |
| Example 731 | 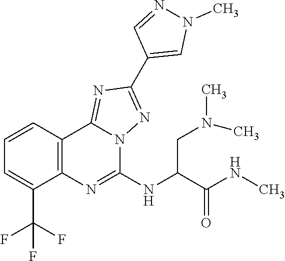 [SMILES: CNC(=O)C(CN(C)C)Nc1nc2c(C(F)(F)F)cccc2c2nc(-c3cnn(C)c3)nn12] |
| | (+/−)-3-(dimethylamino)-N-methyl-N²-[2-(1-methyl-1H-pyrazol-4-yl)-7- |
| | (trifluoromethyl)[1,2,4]triazolo[1,5-c]quinazolin-5-yl]-alaninamide |
| | LC-MS (Method 1): Rₜ = 0.77 min; MS (ESIneg): m/z = 460 [M − H]⁻ |
| | ¹H-NMR (400 MHz, DMSO-d₆): δ [ppm] = 2.23 (s, 6H), 2.61 (d, 3H), 2.70- |
| | 2.80 (m, 1H), 2.84-2.96 (m, 1H), 3.97 (s, 3H), 4.77 (dd, 1H), 7.53 (t, 1H), |
| | 7.88 (br d, 1H), 8.06-8.09 (m, 2H), 8.17 (q, 1H), 8.48-8.52 (m, 2H). |
| Example 732 | 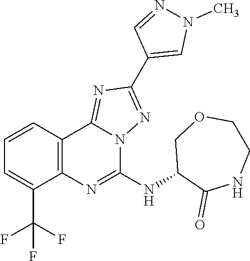 [SMILES: Cn1cc(-c2nc3c4cccc(C(F)(F)F)c4nc(N[C@@H]4COCCNC4=O)n3n2)cn1] |
| | (6R)-6-{[2-(1-methyl-1H-pyrazol-4-yl)-7- |
| | (trifluoromethyl)[1,2,4]triazolo[1,5-c]quinazolin-5-yl]amino}-1,4- |
| | oxazepan-5-one |
| | LC-MS (Method 1): Rₜ = 1.08 min; MS (ESIneg): m/z = 445 [M − H]⁻ |
| | ¹H-NMR (400 MHz, DMSO-d₆): δ [ppm] = 3.13-3.21 (m, 1H), 3.35-3.43 (m, |
| | 1H), 3.49-3.58 (m, 2H), 3.96 (s, 3H), 3.99 (dd, 1H), 4.29 (dd, 1H), 4.96 (td, |
| | 1H), 7.56 (t, 1H), 7.80 (d, 1H), 8.07 (d, 1H), 8.10 (dd, 1H), 8.47 (dd, 1H), |
| | 8.49-8.53 (m, 2H). |
| Example 733 | 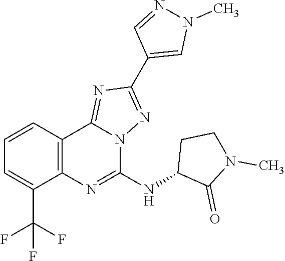 [SMILES: CN1CC[C@@H](Nc2nc3c(C(F)(F)F)cccc3c3nc(-c4cnn(C)c4)nn23)C1=O] |
| | (3R)-1-methyl-3-{[2-(1-methyl-1H-pyrazol-4-yl)-7- |
| | (trifluoromethyl)[1,2,4]triazolo[1,5-c]quinazolin-5-yl]amino}pyrrolidin-2- |
| | one |
| | LC-MS (Method 1): Rₜ = 1.07 min; MS (ESIneg): m/z = 429 [M − H]⁻ |
| | ¹H-NMR (400 MHz, DMSO-d₆): δ [ppm] = 2.34-2.42 (m, 1H), 2.47-2.53 (m, |
| | 1H, partial in DMSO signal), 2.80 (s, 3H), 3.36-3.48 (m, 2H), 3.97 (s, 3H), |
| | 4.61-4.69 (m, 1H), 7.52 (t, 1H), 8.03-8.08 (m, 2H), 8.42 (s, 1H), 8.48 (dd, |
| | 1H), 8.70 (d, 1H). |
| Example 734 | 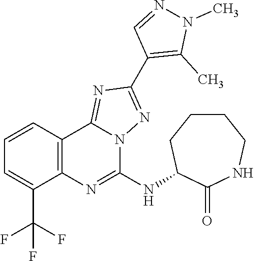 [SMILES: Cc1c(-c2nc3c4cccc(C(F)(F)F)c4nc(N[C@@H]4CCCCNC4=O)n3n2)cnn1C] |
| | (3R)-3-{[2-(1,5-dimethyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 2): Rₜ = 1.29 min; MS (ESIneg): m/z = 457 [M − H]⁻ |
| | ¹H-NMR (400 MHz, DMSO-d₆): δ [ppm] = 1.25-1.38 (m, 1H), 1.48-1.59 (m, |
| | 1H), 1.79-1.91 (m, 2H), 1.97-2.07 (m, 1H), 2.36-2.44 (m, 1H), 2.75 (s, |
| | 3H), 3.15-3.30 (m, 2H), 3.84 (s, 3H), 4.75 (br dd, 1H), 7.53 (t, 1H), 7.82 (br |
| | d, 1H), 7.99 (s, 1H), 8.08 (br d, 1H), 8.23 (br t, 1H), 8.51 (d, 1H). |
| Example 735 | 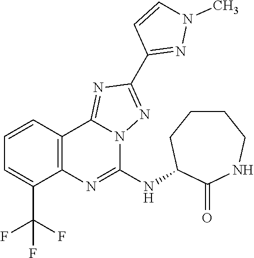 [SMILES: Cn1ccc(-c2nc3c4cccc(C(F)(F)F)c4nc(N[C@@H]4CCCCNC4=O)n3n2)n1] |
| | (3R)-3-{[2-(1-methyl-1H-pyrazol-3-yl)-7- |
| | (trifluoromethyl)[1,2,4]triazolo[1,5-c]quinazolin-5-yl]amino}azepan-2- |
| | one |
| | LC-MS (Method 2): Rₜ = 1.21 min; MS (ESIneg): m/z = 443 [M − H]⁻ |
| | ¹H-NMR (400 MHz, DMSO-d₆): δ [ppm] = 1.25-1.40 (m, 1H), 1.46-1.59 (m, |
| | 1H), 1.78-1.92 (m, 2H), 1.97-2.07 (m, 1H), 2.36-2.46 (m, 1H), 3.15-3.31 |
| | (m, 2H), 3.99 (s, 3H), 4.75 (br dd, 1H), 6.93 (d, 1H), 7.55 (t, 1H), 7.86 (d, |
| | 1H), 7.90 (d, 1H), 8.10 (d, 1H), 8.28 (br t, 1H), 8.53 (d, 1H). |
| Example 736 | 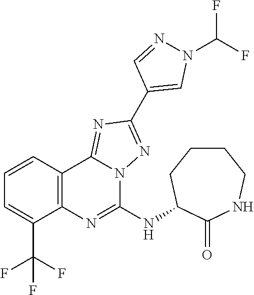 [SMILES: O=C1NCCCC[C@H]1Nc1nc2c(C(F)(F)F)cccc2c2nc(-c3cnn(C(F)F)c3)nn12] |
| | (3R)-3-({2-[1-(difluoromethyl)-1H-pyrazol-4-yl]-7- |
| | (trifluoromethyl)[1,2,4]triazolo[1,5-c]quinazolin-5-yl}amino)azepan-2- |
| | one |
| | LC-MS (Method 2): Rₜ = 1.37 min; MS (ESIneg): m/z = 479 [M − H]⁻ |
| | ¹H-NMR (400 MHz, DMSO-d₆): δ [ppm] = 1.27-1.38 (m, 1H), 1.47-1.58 (m, |
| | 1H), 1.79-1.91 (m, 2H), 2.03 (br d, 1H), 2.37-2.44 (m, 1H), 3.16-3.31 (m, |
| | 2H), 4.73-4.79 (m, 1H), 7.56 (t, 1H), 7.82 (br d, 1H), 7.93 (t, 1H), 8.11 (dd, |
| | 1H), 8.27 (br dd, 1H), 8.46 (s, 1H), 8.52 (dd, 1H), 9.04 (s, 1H). |
| Example 737 | 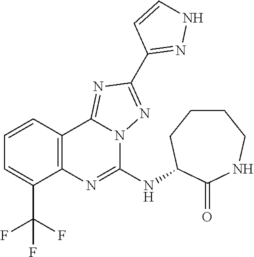 [SMILES: O=C1NCCCC[C@H]1Nc1nc2c(C(F)(F)F)cccc2c2nc(-c3cc[nH]n3)nn12] |
| | (3R)-3-{[2-(1H-pyrazol-3-yl)-7-(trifluoromethyl)[1,2,4]triazolo[1,5- |
| | c]quinazolin-5-yl]amino}azepan-2-one |
| | LC-MS (Method 2): Rₜ = 1.20 min; MS (ESIneg): m/z = 429 [M − H]⁻ |
| | ¹H-NMR (400 MHz, DMSO-d₆): δ [ppm] = 1.26-1.41 (m, 1H), 1.45-1.59 (m, |
| | 1H), 1.79-1.92 (m, 2H), 2.03 (br d, 1H), 2.38-2.46 (m, 1H), 3.15-3.31 (m, |
| | 2H), 4.77 (br dd, 1H), 6.97 (br s, 1H), 7.56 (t, 1H), 7.86 (br d, 1H), 7.95 (br s, |
| | 1H), 8.11 (d, 1H), 8.23-8.31 (m, 1H), 8.55 (dd, 1H), 13.39 (br s, 1H). |
| Example 738 | 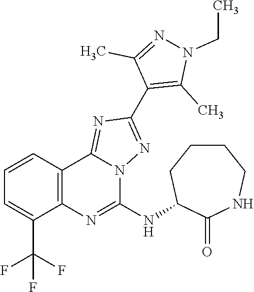 [SMILES: CCn1nc(C)c(-c2nc3c4cccc(C(F)(F)F)c4nc(N[C@@H]4CCCCNC4=O)n3n2)c1C] |
| | (3R)-3-{[2-(1-ethyl-3,5-dimethyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 2): Rₜ = 1.48 min; MS (ESIpos): m/z = 487 [M + H]⁺ |
| | ¹H-NMR (400 MHz, DMSO-d₆): δ [ppm] = 1.29-1.37 (m, 1H), 1.34 (t, 3H), |
| | 1.47-1.60 (m, 1H), 1.80-1.91 (m, 2H), 2.03 (br d, 1H), 2.38-2.46 (m, 1H), |
| | 2.55 (s, 3H), 2.73 (s, 3H), 3.16-3.31 (m, 2H), 4.10 (q, 2H), 4.74 (dd, 1H), |
| | 7.53 (t, 1H), 7.85 (d, 1H), 8.09 (dd, 1H), 8.20-8.26 (m, 1H), 8.52 (dd, 1H). |
| Example 739 | 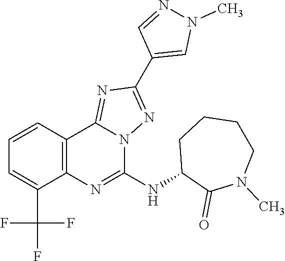 [SMILES: CN1CCCC[C@@H](Nc2nc3c(C(F)(F)F)cccc3c3nc(-c4cnn(C)c4)nn23)C1=O] |
| | (3R)-1-methyl-3-{[2-(1-methyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 1): Rₜ = 1.33 min; MS (ESIneg): m/z = 457 [M − H]⁻ |
| | ¹H-NMR (400 MHz, DMSO-d₆): δ [ppm] = 1.38-1.55 (m, 2H), 1.80-1.93 (m, |
| | 2H), 1.94-2.02 (m, 1H), 2.33-2.40 (m, 1H), 3.01 (s, 3H), 3.32-3.40 (m, |
| | 1H, partial in water signal), 3.66 (dd, 1H), 3.96 (s, 3H), 4.90 (dd, 1H), 7.54 (t, |
| | 1H), 7.88 (d, 1H), 8.07-8.11 (m, 2H), 8.47-8.52 (m, 2H). |
| Example 740 | 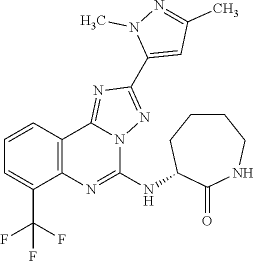 [SMILES: Cc1cc(-c2nc3c4cccc(C(F)(F)F)c4nc(N[C@@H]4CCCCNC4=O)n3n2)n(C)n1] |
| | (3R)-3-{[2-(1,3-dimethyl-1H-pyrazol-5-yl)-7- |
| | (trifluoromethyl)[1,2,4]triazolo[1,5-c]quinazolin-5-yl]amino}azepan-2- |
| | one |
| | LC-MS (Method 2): Rₜ = 1.34 min; MS (ESIneg): m/z = 457 [M − H]⁻ |
| | ¹H-NMR (500 MHz, DMSO-d₆): δ [ppm] = 1.27-1.37 (m, 1H), 1.51-1.60 (m, |
| | 1H), 1.80-1.90 (m, 2H), 2.03 (br d, 1H), 2.24 (s, 3H), 2.37-2.43 (m, 1H), |
| | 3.17-3.31 (m, 2H), 4.26 (s, 3H), 4.76 (dd, 1H), 6.83 (s, 1H), 7.56 (t, 1H), |
| | 7.92 (d, 1H), 8.12 (dd, 1H), 8.25 (dd, 1H), 8.54 (dd, 1H). |
| Example 741 | 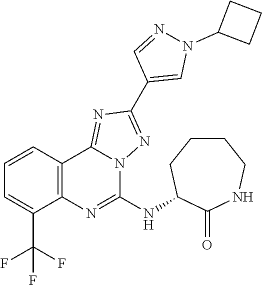 [SMILES: O=C1NCCCC[C@H]1Nc1nc2c(C(F)(F)F)cccc2c2nc(-c3cnn(C4CCC4)c3)nn12] |
| | (3R)-3-{[2-(1-cyclobutyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 2): Rₜ = 1.38 min; MS (ESIneg): m/z = 483 [M − H]⁻ |
| | ¹H-NMR (400 MHz, DMSO-d₆): δ [ppm] = 1.25-1.38 (m, 1H), 1.45-1.59 (m, |
| | 1H), 1.74-1.92 (m, 4H), 1.94-2.07 (m, 1H), 2.35-2.47 (m, 3H), 2.52-2.63 |
| | (m, 2H), 3.13-3.30 (m, 2H), 4.75 (dd, 1H), 4.97 (tt, 1H), 7.54 (t, 1H), 7.77 |
| | (d, 1H), 8.09 (dd, 1H), 8.13 (d, 1H), 8.27 (dd, 1H), 8.49 (dd, 1H), 8.61 (d, |
| | 1H). |
| Example 742 | 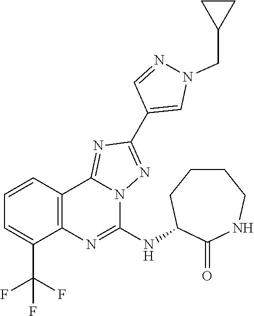 [SMILES: O=C1NCCCC[C@H]1Nc1nc2c(C(F)(F)F)cccc2c2nc(-c3cnn(CC4CC4)c3)nn12] |
| | (3R)-3-({2-[1-(cyclopropylmethyl)-1H-pyrazol-4-yl]-7- |
| | (trifluoromethyl)[1,2,4]triazolo[1,5-c]quinazolin-5-yl}amino)azepan-2- |
| | one |
| | LC-MS (Method 2): Rₜ = 1.35 min; MS (ESIneg): m/z = 483 [M − H]⁻ |
| | ¹H-NMR (400 MHz, DMSO-d₆): δ [ppm] = 0.41-0.47 (m, 2H), 0.54-0.60 (m, |
| | 2H), 1.26-1.38 (m, 2H), 1.46-1.59 (m, 1H), 1.79-1.91 (m, 2H), 1.97-2.07 |
| | (m, 1H), 2.37-2.44 (m, 1H), 3.15-3.30 (m, 2H), 4.09 (d, 2H), 4.76 (dd, 1H), |
| | 7.54 (t, 1H), 7.78 (d, 1H), 8.08-8.12 (m, 2H), 8.26 (dd, 1H), 8.51 (dd, 1H), |
| | 8.57 (d, 1H). |
| Example 743 | 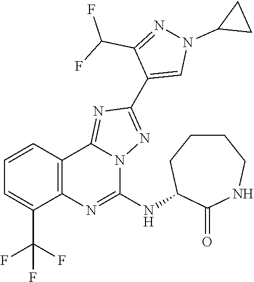 [SMILES: O=C1NCCCC[C@H]1Nc1nc2c(C(F)(F)F)cccc2c2nc(-c3cn(C4CC4)nc3C(F)F)nn12] |
| | (3R)-3-({2-[1-cyclopropyl-3-(difluoromethyl)-1H-pyrazol-4-yl]-7- |
| | (trifluoromethyl)[1,2,4]triazolo[1,5-c]quinazolin-5-yl}amino)azepan-2- |
| | one |
| | LC-MS (Method 2): Rₜ = 1.40 min; MS (ESIneg): m/z = 519 [M − H]⁻ |
| | ¹H-NMR (400 MHz, DMSO-d₆): δ [ppm] = 1.03-1.10 (m, 2H), 1.20-1.25 (m, |
| | 2H), 1.27-1.39 (m, 1H), 1.47-1.62 (m, 1H), 1.82-1.90 (m, 2H), 2.03 (br d, |
| | 1H), 2.34-2.42 (m, 1H), 3.15-3.31 (m, 2H), 3.97 (tt, 1H), 4.78 (br dd, 1H), |
| | 7.55 (t, 1H), 7.69 (t, 1H), 7.91 (d, 1H), 8.11 (dd, 1H), 8.19-8.26 (m, 1H), |
| | 8.52 (dd, 1H), 8.72 (s, 1H). |
| Example 744 | 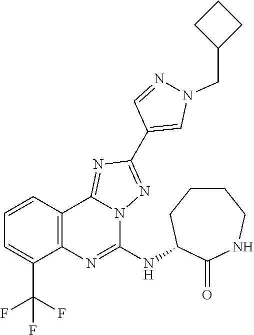 [SMILES: O=C1NCCCC[C@H]1Nc1nc2c(C(F)(F)F)cccc2c2nc(-c3cnn(CC4CCC4)c3)nn12] |
| | (3R)-3-({2-[1-(cyclobutylmethyl)-1H-pyrazol-4-yl]-7- |
| | (trifluoromethyl)[1,2,4]triazolo[1,5-c]quinazolin-5-yl}amino)azepan-2- |
| | one |
| | LC-MS (Method 2): Rₜ = 1.42 min; MS (ESIneg): m/z = 497 [M − H]⁻ |
| | ¹H-NMR (400 MHz, DMSO-d₆): δ [ppm] = 1.26-1.38 (m, 1H), 1.46-1.58 (m, |
| | 1H), 1.76-1.93 (m, 6H), 1.96-2.05 (m, 3H), 2.34-2.46 (m, 1H), 2.83 (dt, |
| | 1H), 3.15-3.32 (m, 2H), 4.25 (d, 2H), 4.75 (br dd, 1H), 7.54 (t, 1H), 7.77 (d, |
| | 1H), 8.07-8.11 (m, 2H), 8.27 (dd, 1H), 8.50 (dd, 1H), 8.52 (d, 1H). |
| Example 745 | 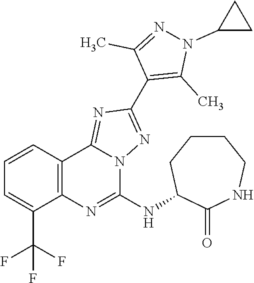 [SMILES: Cc1nn(C2CC2)c(C)c1-c1nc2c3cccc(C(F)(F)F)c3nc(N[C@@H]3CCCCNC3=O)n2n1] |
| | (3R)-3-{[2-(1-cyclopropyl-3,5-dimethyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 2): Rₜ = 1.52 min; MS (ESIneg): m/z = 497 [M − H]⁻ |
| | ¹H-NMR (400 MHz, DMSO-d₆): δ [ppm] = 1.00-1.12 (m, 4H), 1.26-1.37 (m, |
| | 1H), 1.48-1.59 (m, 1H), 1.79-1.91 (m, 2H), 1.98-2.07 (m, 1H), 2.39-2.46 |
| | (m, 1H), 2.51 (s, 3H), 2.79 (s, 3H), 3.16-3.30 (m, 2H), 3.51-3.57 (m, 1H), |
| | 4.73 (br dd, 1H), 7.52 (t, 1H), 7.83 (d, 1H), 8.08 (dd, 1H), 8.23 (dd, 1H), 8.49 |
| | (dd, 1H). |
| Example 746 | 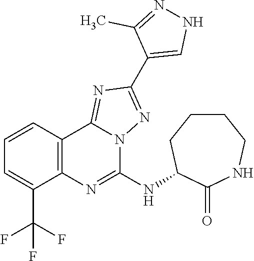 [SMILES: Cc1n[nH]cc1-c1nc2c3cccc(C(F)(F)F)c3nc(N[C@@H]3CCCCNC3=O)n2n1] |
| | (3R)-3-{[2-(3-methyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 1): Rₜ = 1.20 min; MS (ESIpos): m/z = 445 [M + H]⁺ |
| | ¹H-NMR (400 MHz, DMSO-d₆): δ [ppm] = 1.26-1.40 (m, 1H), 1.46-1.61 (m, |
| | 1H), 1.78-1.92 (m, 2H), 2.02 (br d, 1H), 2.37-2.46 (m, 1H), 2.67 (s, 3H), |
| | 3.17-3.27 (m, 2H), 4.75 (br dd, 1H), 7.53 (t, 1H), 7.81 (d, 1H), 8.09 (dd, |
| | 1H), 8.16 (br s, 1H), 8.24 (dd, 1H), 8.51 (dd, 1H), 13.00 (br s, 1H). |
| Example 747 | 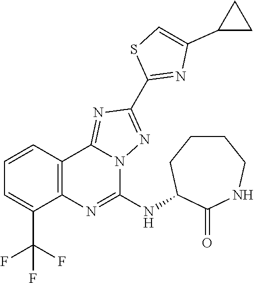 [SMILES: O=C1NCCCC[C@H]1Nc1nc2c(C(F)(F)F)cccc2c2nc(-c3nc(C4CC4)cs3)nn12] |
| | (3R)-3-{[2-(4-cyclopropyl-1,3-thiazol-2-yl)-7- |
| | (trifluoromethyl)[1,2,4]triazolo[1,5-c]quinazolin-5-yl]amino}azepan-2- |
| | one |
| | LC-MS (Method 1): Rₜ = 1.47 min; MS (ESIpos): m/z = 488 [M + H]⁺ |
| | ¹H-NMR (400 MHz, DMSO-d₆): δ [ppm] = 0.91-1.04 (m, 4H), 1.27-1.40 (m, |
| | 1H), 1.48-1.61 (m, 1H), 1.78-1.91 (m, 2H), 1.97-2.07 (m, 1H), 2.21-2.28 |
| | (m, 1H), 2.37-2.44 (m, 1H), 3.16-3.31 (m, 2H), 4.77 (br dd, 1H), 7.57 (t, |
| | 1H), 7.63 (s, 1H), 7.92 (d, 1H), 8.13 (dd, 1H), 8.28 (dd, 1H), 8.56 (dd, 1H). |
| Example 748 | 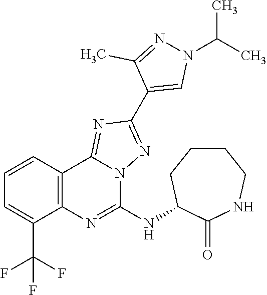 [SMILES: Cc1nn(C(C)C)cc1-c1nc2c3cccc(C(F)(F)F)c3nc(N[C@@H]3CCCCNC3=O)n2n1] |
| | (3R)-3-({2-[3-methyl-1-(propan-2-yl)-1H-pyrazol-4-yl]-7- |
| | (trifluoromethyl)[1,2,4]triazolo[1,5-c]quinazolin-5-yl}amino)azepan-2- |
| | one |
| | LC-MS (Method 1): Rₜ = 1.43 min; MS (ESIpos): m/z = 487 [M + H]⁺ |
| | ¹H-NMR (400 MHz, DMSO-d₆): δ [ppm] = 1.25-1.39 (m, 1H), 1.47 (d, 6H), |
| | 1.48-1.58 (m, 1H), 1.80-1.91 (m, 2H), 1.97-2.06 (m, 1H), 2.38-2.46 (m, |
| | 1H), 2.60 (s, 3H), 3.15-3.31 (m, 2H), 4.55 (spt, 1H), 4.75 (dd, 1H), 7.54 (t, |
| | 1H), 7.81 (d, 1H), 8.09 (dd, 1H), 8.25 (dd, 1H), 8.42 (s, 1H), 8.49 (dd, 1H). |
| Example 749 | 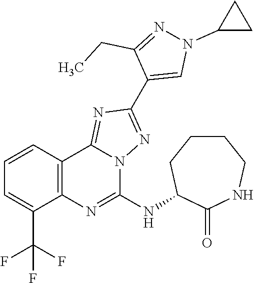 [SMILES: CCc1nn(C2CC2)cc1-c1nc2c3cccc(C(F)(F)F)c3nc(N[C@@H]3CCCCNC3=O)n2n1] |
| | (3R)-3-{[2-(1-cyclopropyl-3-ethyl-1H-pyrazol-4-yl)-7- |
| | (trifluoromethyl)[1,2,4]triazolo[1,5-c]quinazolin-5-yl]amino}azepan-2- |
| | one |
| | LC-MS (Method 1): Rₜ = 1.47 min; MS (ESIneg): m/z = 497 [M − H]⁻ |
| | ¹H-NMR (400 MHz, DMSO-d₆): δ [ppm] = 0.95-1.01 (m, 2H), 1.11-1.16 (m, |
| | 2H), 1.26-1.37 (m, 1H), 1.29 (t, 3H), 1.45-1.58 (m, 1H), 1.79-1.91 (m, |
| | 2H), 2.02 (br d, 1H), 2.38-2.46 (m, 1H), 3.04 (q, 2H), 3.15-3.31 (m, 2H), |
| | 3.78 (tt, 1H), 4.73 (dd, 1H), 7.54 (t, 1H), 7.79 (d, 1H), 8.09 (dd, 1H), 8.24 |
| | (dd, 1H), 8.41 (s, 1H), 8.47 (dd, 1H). |
| Example 750 | 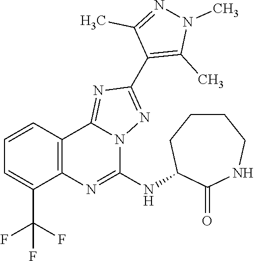 [SMILES: Cc1nn(C)c(C)c1-c1nc2c3cccc(C(F)(F)F)c3nc(N[C@@H]3CCCCNC3=O)n2n1] |
| | (3R)-3-{[7-(trifluoromethyl)-2-(1,3,5-trimethyl-1H-pyrazol-4- |
| | yl)[1,2,4]triazolo[1,5-c]quinazolin-5-yl]amino}azepan-2-one |
| | LC-MS (Method 2): Rₜ = 1.39 min; MS (ESIneg): m/z = 471 [M − H]⁻ |
| | ¹H-NMR (400 MHz, DMSO-d₆): δ [ppm] = 1.25-1.39 (m, 1H), 1.48-1.59 (m, |
| | 1H), 1.79-1.91 (m, 2H), 1.98-2.07 (m, 1H), 2.37-2.46 (m, 1H), 2.53 (s, |
| | 3H), 2.71 (s, 3H), 3.16-3.30 (m, 2H), 3.75 (s, 3H), 4.73 (dd, 1H), 7.53 (t, |
| | 1H), 7.84 (d, 1H), 8.09 (dd, 1H), 8.22 (dd, 1H), 8.50 (dd, 1H). |
| Example 751 | 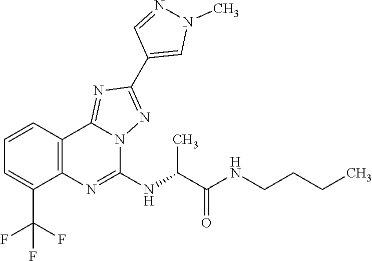 [SMILES: CCCCNC(=O)[C@@H](C)Nc1nc2c(C(F)(F)F)cccc2c2nc(-c3cnn(C)c3)nn12] |
| | N-butyl-N²-[2-(1-methyl-1H-pyrazol-4-yl)-7- |
| | (trifluoromethyl)[1,2,4]triazolo[1,5-c]quinazolin-5-yl]-D-alaninamid |
| | LC-MS (Method 1): Rₜ = 1.26 min; MS (ESIneg): m/z = 461 [M − H]⁻ |
| | ¹H-NMR (400 MHz, DMSO-d₆): δ [ppm] = 0.85 (t, 3H), 1.21-1.32 (m, 2H), |
| | 1.35-1.44 (m, 2H), 1.54 (d, 3H), 3.03-3.16 (m, 2H), 3.96 (s, 3H), 4.70 (dq, |
| | 1H), 7.53 (t, 1H), 7.95 (d, 1H), 8.05-8.10 (m, 2H), 8.18 (t, 1H), 8.47-8.51 |
| | (m, 2H). |
| Example 752 | 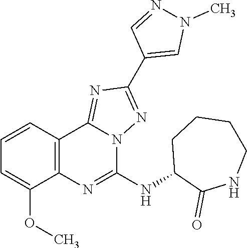 [SMILES: COc1cccc2c1nc(N[C@@H]1CCCCNC1=O)n1nc(-c3cnn(C)c3)nc21] |
| | (3R)-3-{[7-methoxy-2-(1-methyl-1H-pyrazol-4-yl)[1,2,4]triazolo[1,5- |
| | c]quinazolin-5-yl]amino}azepan-2-one |
| | LC-MS (Method 1): Rₜ = 0.98 min; MS (ESIneg): m/z = 405 [M − H]⁻ |
| | ¹H-NMR (400 MHz, DMSO-d₆): δ [ppm] = 1.25-1.39 (m, 1H), 1.46-1.58 (m, |
| | 1H), 1.79-1.91 (m, 2H), 1.98-2.07 (m, 1H), 2.32-2.38 (m, 1H), 3.11-3.22 |
| | (m, 1H), 3.28-3.39 (m, 1H, partial in water signal), 3.95 (s, 3H), 3.96 (s, |
| | 3H), 4.78-4.84 (m, 1H), 7.29 (dd, 1H), 7.36 (t, 1H), 7.54 (d, 1H), 7.82 (dd, |
| | 1H), 8.06 (d, 1H), 8.21 (dd, 1H), 8.49 (d, 1H). |
| Example 753 | 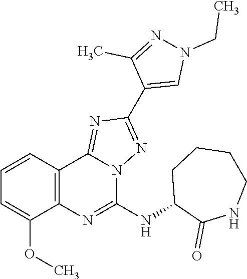 [SMILES: CCn1cc(-c2nc3c4cccc(OC)c4nc(N[C@@H]4CCCCNC4=O)n3n2)c(C)n1] |
| | (3R)-3-{[2-(1-ethyl-3-methyl-1H-pyrazol-4-yl)-7- |
| | methoxy[1,2,4]triazolo[1,5-c]quinazolin-5-yl]amino}azepan-2-one |
| | LC-MS (Method 1): Rₜ = 1.11 min; MS (ESIneg): m/z = 433 [M − H]⁻ |
| | ¹H-NMR (400 MHz, DMSO-d₆): δ [ppm] = 1.26-1.38 (m, 1H), 1.41 (t, 3H), |
| | 1.47-1.57 (m, 1H), 1.80-1.92 (m, 2H), 1.96-2.07 (m, 1H), 2.33-2.42 (m, |
| | 1H), 2.58 (s, 3H), 3.12-3.21 (m, 1H), 3.28-3.38 (m, 1H, partial in water |
| | signal), 3.96 (s, 3H), 4.16 (q, 2H), 4.78-4.83 (m, 1H), 7.29 (dd, 1H), 7.36 (t, |
| | 1H), 7.59 (d, 1H), 7.81 (dd, 1H), 8.19 (dd, 1H), 8.41 (s, 1H). |
| Example 754 | 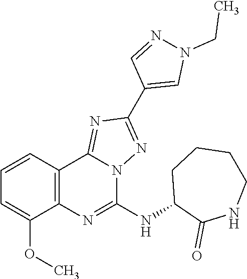 [SMILES: CCn1cc(-c2nc3c4cccc(OC)c4nc(N[C@@H]4CCCCNC4=O)n3n2)cn1] |
| | (3R)-3-{[2-(1-ethyl-1H-pyrazol-4-yl)-7-methoxy[1,2,4]triazolo[1,5- |
| | c]quinazolin-5-yl]amino}azepan-2-one |
| | LC-MS (Method 1): Rₜ = 1.01 min; MS (ESIpos): m/z = 421 [M + H]⁺ |
| | ¹H-NMR (500 MHz, DMSO-d₆): δ [ppm] = 1.28-1.37 (m, 1H), 1.44 (t, 3H), |
| | 1.47-1.56 (m, 1H), 1.81-1.91 (m, 2H), 2.03 (br d, 1H), 2.32-2.38 (m, 1H), |
| | 3.13-3.20 (m, 1H), 3.29-3.37 (m, 1H, partial in water signal), 3.96 (s, 3H), |
| | 4.25 (q, 2H), 4.79-4.84 (m, 1H), 7.29 (dd, 1H), 7.37 (t, 1H), 7.54 (d, 1H), |
| | 7.82 (dd, 1H), 8.08 (d, 1H), 8.21 (dd, 1H), 8.53 (d, 1H). |
| Example 755 | 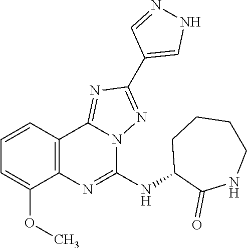 [SMILES: COc1cccc2c1nc(N[C@@H]1CCCCNC1=O)n1nc(-c3cn[nH]c3)nc21] |
| | (3R)-3-{[7-methoxy-2-(1H-pyrazol-4-yl)[1,2,4]triazolo[1,5-c]quinazolin-5- |
| | yl]amino}azepan-2-one |
| | LC-MS (Method 1): Rₜ = 0.88 min; MS (ESIpos): m/z = 393 [M + H]⁺ |
| | ¹H-NMR (400 MHz, DMSO-d₆): δ [ppm] = 1.26-1.38 (m, 1H), 1.46-1.59 (m, |
| | 1H), 1.79-1.93 (m, 2H), 1.96-2.09 (m, 1H), 2.32-2.38 (m, 1H), 3.12-3.21 |
| | (m, 1H), 3.29-3.38 (m, 1H, partial in water signal), 3.96 (s, 3H), 4.79-4.85 |
| | (m, 1H), 7.29 (dd, 1H), 7.34-7.39 (m, 1H), 7.55 (d, 1H), 7.83 (dd, 1H), 8.13 |
| | (br s, 1H), 8.20 (dd, 1H), 8.49 (br s, 1H), 13.35 (br s, 1H). |
| Example 756 | 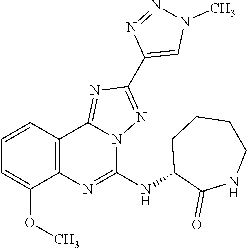 [SMILES: COc1cccc2c1nc(N[C@@H]1CCCCNC1=O)n1nc(-c3cn(C)nn3)nc21] |
| | (3R)-3-{[7-methoxy-2-(1-methyl-1H-1,2,3-triazol-4-yl)[1,2,4]triazolo[1,5- |
| | c]quinazolin-5-yl]amino}azepan-2-one |
| | LC-MS (Method 1): Rₜ = 0.92 min; MS (ESIpos): m/z = 408 [M + H]⁺ |
| | ¹H-NMR (400 MHz, DMSO-d₆): δ [ppm] = 1.26-1.39 (m, 1H), 1.47-1.58 (m, |
| | 1H), 1.81-1.92 (m, 2H), 1.98-2.08 (m, 1H), 2.34-2.42 (m, 1H), 3.13-3.22 |
| | (m, 1H), 3.29-3.39 (m, 1H, partial in water signal), 3.97 (s, 3H), 4.17 (s, |
| | 3H), 4.79-4.85 (m, 1H), 7.32 (dd, 1H), 7.39 (t, 1H), 7.66 (d, 1H), 7.84 (dd, |
| | 1H), 8.24 (dd, 1H), 8.86 (s, 1H). |
| Example 757 | 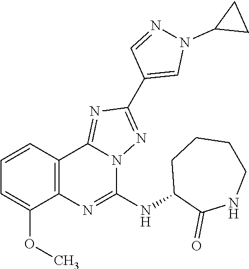 [SMILES: COc1cccc2c1nc(N[C@@H]1CCCCNC1=O)n1nc(-c3cnn(C4CC4)c3)nc21] |
| | (3R)-3-{[2-(1-cyclopropyl-1H-pyrazol-4-yl)-7-methoxy[1,2,4]triazolo[1,5- |
| | c]quinazolin-5-yl]amino}azepan-2-one |
| | LC-MS (Method 1): Rₜ = 1.08 min; MS (ESIneg): m/z = 431 [M − H]⁻ |
| | ¹H-NMR (400 MHz, DMSO-d₆): δ [ppm] = 0.98-1.05 (m, 2H), 1.15-1.20 (m, |
| | 2H), 1.26-1.38 (m, 1H), 1.46-1.57 (m, 1H), 1.80-1.92 (m, 2H), 1.97-2.07 |
| | (m, 1H), 2.31-2.38 (m, 1H), 3.12-3.21 (m, 1H), 3.28-3.38 (m, 1H, partial |
| | in water signal), 3.88 (tt, 1H), 3.96 (s, 3H), 4.79-4.84 (m, 1H), 7.29 (dd, |
| | 1H), 7.37 (t, 1H), 7.54 (d, 1H), 7.82 (dd, 1H), 8.06 (d, 1H), 8.21 (dd, 1H), |
| | 8.54 (d, 1H). |
| Example 758 | 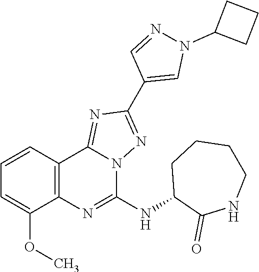 [SMILES: COc1cccc2c1nc(N[C@@H]1CCCCNC1=O)n1nc(-c3cnn(C4CCC4)c3)nc21] |
| | (3R)-3-{[2-(1-cyclobutyl-1H-pyrazol-4-yl)-7-methoxy[1,2,4]triazolo[1,5- |
| | c]quinazolin-5-yl]amino}azepan-2-one |
| | LC-MS (Method 1): Rₜ = 1.16 min; MS (ESIneg): m/z = 445 [M − H]⁻ |
| | ¹H-NMR (400 MHz, DMSO-d₆): δ [ppm] = 1.26-1.40 (m, 1H), 1.45-1.58 (m, |
| | 1H), 1.76-1.92 (m, 4H), 1.96-2.07 (m, 1H), 2.32-2.47 (m, 3H), 2.52-2.62 |
| | (m, 2H), 3.12-3.21 (m, 1H), 3.28-3.39 (m, 1H, partial in water signal), 3.96 |
| | (s, 3H), 4.78-4.85 (m, 1H), 4.97 (tt, 1H), 7.29 (dd, 1H), 7.37 (t, 1H), 7.54 (d, |
| | 1H), 7.82 (dd, 1H), 8.11 (d, 1H), 8.21 (dd, 1H), 8.59 (d, 1H). |
| Example 759 | 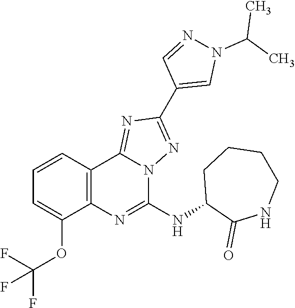 [SMILES: CC(C)n1cc(-c2nc3c4cccc(OC(F)(F)F)c4nc(N[C@@H]4CCCCNC4=O)n3n2)cn1] |
| | (3R)-3-({2-[1-(propan-2-yl)-1H-pyrazol-4-yl]-7- |
| | (trifluoromethoxy)[1,2,4]triazolo[1,5-c]quinazolin-5-yl}amino)azepan-2- |
| | one |
| | LC-MS (Method 1): Rₜ = 1.32 min; MS (ESIneg): m/z = 487 [M − H]⁻ |
| | ¹H-NMR (400 MHz, DMSO-d₆): δ [ppm] = 1.25-1.40 (m, 1H), 1.50 (d, 6H), |
| | 1.47-1.58 (m, 1H), 1.76-1.90 (m, 2H), 1.96-2.07 (m, 1H), 2.37 (br d, 1H), |
| | 3.13-3.22 (m, 1H), 3.23-3.30 (m, 1H), 4.64 (spt, 1H), 4.78 (dd, 1H), 7.48 |
| | (t, 1H), 7.75-7.80 (m, 2H), 8.10 (d, 1H), 8.23-8.30 (m, 2H), 8.55 (d, 1H). |
| Example 760 | 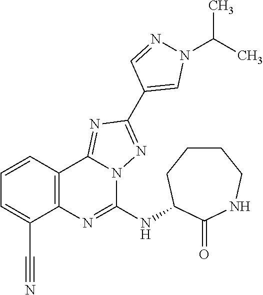 [SMILES: CC(C)n1cc(-c2nc3c4cccc(C#N)c4nc(N[C@@H]4CCCCNC4=O)n3n2)cn1] |
| | 5-{[(3R)-2-oxoazepan-3-yl]amino}-2-[1-(propan-2-yl)-1H-pyrazol-4- |
| | yl][1,2,4]triazolo[1,5-c]quinazoline-7-carbonitrile |
| | LC-MS (Method 1): Rₜ = 1.16 min; MS (ESIneg): m/z = 428 [M − H]⁻ |
| | ¹H-NMR (400 MHz, DMSO-d₆): δ [ppm] = 1.28-1.40 (m, 1H), 1.50 (d, 6H), |
| | 1.50-1.60 (m, 1H), 1.83-1.98 (m, 2H), 2.00-2.08 (m, 1H), 2.37-2.44 (m, |
| | 1H), 3.14-3.23 (m, 1H), 3.25-3.32 (m, 1H), 4.64 (spt, 1H), 4.80-4.86 (m, |
| | 1H), 7.53 (t, 1H), 7.87 (d, 1H), 8.10 (d, 1H), 8.21 (dd, 1H), 8.27 (dd, 1H), |
| | 8.50 (dd, 1H), 8.55 (d, 1H). |
| Example 761 | 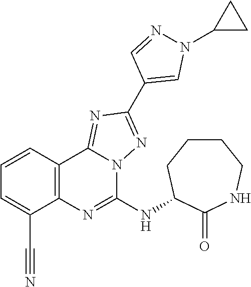 [SMILES: N#Cc1cccc2c1nc(N[C@@H]1CCCCNC1=O)n1nc(-c3cnn(C4CC4)c3)nc21] |
| | 2-(1-cyclopropyl-1H-pyrazol-4-yl)-5-{[(3R)-2-oxoazepan-3- |
| | yl]amino}[1,2,4]triazolo[1,5-c]quinazoline-7-carbonitrile |
| | LC-MS (Method 1): Rₜ = 1.12 min; MS (ESIneg): m/z = 426 [M − H]⁻ |
| | ¹H-NMR (400 MHz, DMSO-d₆): δ [ppm] = 0.99-1.06 (m, 2H), 1.15-1.20 (m, |
| | 2H), 1.25-1.39 (m, 1H), 1.49-1.60 (m, 1H), 1.83-1.98 (m, 2H), 2.00-2.08 |
| | (m, 1H), 2.37-2.44 (m, 1H), 3.15-3.23 (m, 1H), 3.25-3.32 (m, 1H), 3.88 |
| | (tt, 1H), 4.82 (br dd, 1H), 7.52 (t, 1H), 7.86 (d, 1H), 8.07 (d, 1H), 8.21 (dd, |
| | 1H), 8.27 (dd, 1H), 8.48 (dd, 1H), 8.56 (d, 1H). |
| Example 762 | 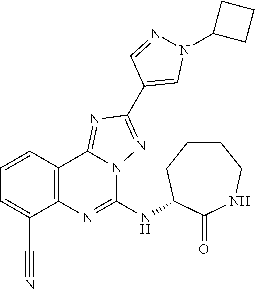 [SMILES: N#Cc1cccc2c1nc(N[C@@H]1CCCCNC1=O)n1nc(-c3cnn(C4CCC4)c3)nc21] |
| | 2-(1-cyclobutyl-1H-pyrazol-4-yl)-5-{[(3R)-2-oxoazepan-3- |
| | yl]amino}[1,2,4]triazolo[1,5-c]quinazoline-7-carbonitrile |
| | LC-MS (Method 1): Rₜ = 1.21 min; MS (ESIneg): m/z = 440 [M − H]⁻ |
| | ¹H-NMR (400 MHz, DMSO-d₆): δ [ppm] = 1.26-1.42 (m, 1H), 1.49-1.61 (m, |
| | 1H), 1.76-1.98 (m, 4H), 2.00-2.08 (m, 1H), 2.38-2.47 (m, 3H), 2.53-2.63 |
| | (m, 2H), 3.15-3.23 (m, 1H), 3.25-3.32 (m, 1H), 4.79-4.86 (m, 1H), 4.97 |
| | (br tt, 1H), 7.52 (t, 1H), 7.87 (d, 1H), 8.13 (d, 1H), 8.21 (dd, 1H), 8.27 (dd, |
| | 1H), 8.49 (dd, 1H), 8.61 (d, 1H). |
| Example 763 | 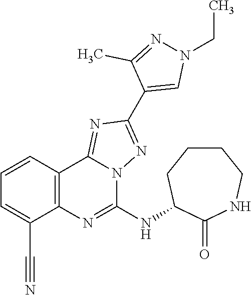 [SMILES: CCn1cc(-c2nc3c4cccc(C#N)c4nc(N[C@@H]4CCCCNC4=O)n3n2)c(C)n1] |
| | 2-(1-ethyl-3-methyl-1H-pyrazol-4-yl)-5-{[(3R)-2-oxoazepan-3- |
| | yl]amino}[1,2,4]triazolo[1,5-c]quinazoline-7-carbonitrile |
| | LC-MS (Method 1): Rₜ = 1.15 min; MS (ESIpos): m/z = 430 [M + H]⁺ |
| | ¹H-NMR (400 MHz, DMSO-d₆): δ [ppm] = 1.26-1.38 (m, 1H), 1.42 (t, 3H), |
| | 1.49-1.62 (m, 1H), 1.83-2.00 (m, 2H), 2.00-2.08 (m, 1H), 2.39-2.46 (m, |
| | 1H), 2.58 (s, 3H), 3.13-3.24 (m, 1H), 3.25-3.32 (m, 1H), 4.16 (q, 2H), 4.78- |
| | 4.85 (m, 1H), 7.52 (t, 1H), 7.90 (d, 1H), 8.21 (dd, 1H), 8.25 (dd, 1H), 8.42 |
| | (s, 1H), 8.47 (dd, 1H). |

### Example 764

### 2-[1-(difluoromethyl)-1H-pyrazol-4-yl]-5-{[(3R)-2-oxoazepan-3-yl]amino}[1,2,4]triazolo[1,5-c]quinazoline-7-carbonitrile

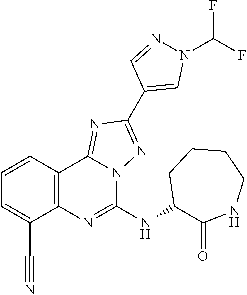

A mixture of 5-chloro-2-[1-(difluoromethyl)-1H-pyrazol-4-yl][1,2,4]triazolo[1,5-c]quinazoline-7-carbonitrile and 5-chloro-2-(1H-pyrazol-4-yl)[1,2,4]triazolo[1,5-c]quinazoline-7-carbonitrile (125 mg, about 362 μmol), (3R)-3-aminoazepan-2-one-hydrogen chloride (1/1) (89.3 mg, 542 μmol) and N,N-diisopropylethylamine (190 μL, 1.1 mmol) were stirred in DMSO (3.4 mL) for 90 minutes at 60° C. The reaction mixture was then cooled to rt and diluted with water. The solid was filtered, washed with water, dried under reduced pressure and purified by HPLC to obtain 12.2 mg (95% purity, 7% yield) of the title product and 10.4 mg (95% purity, 7% yield) of 5-{[(3R)-2-oxoazepan-3-yl]amino}-2-(1H-pyrazol-4-yl)[1,2,4]triazolo[1,5-c]quinazoline-7-carbonitrile

LC-MS (Method 2): Rₜ=1.14 min; MS (ESIpos): m/z=438 [M+H]⁺

¹H-NMR (400 MHz, DMSO-d₆): δ [ppm]=1.27-1.42 (m, 1H), 1.49-1.61 (m, 1H), 1.83-1.99 (m, 2H), 2.00-2.08 (m, 1H), 2.37-2.45 (m, 1H), 3.15-3.24 (m, 1H, partial in water signal), 3.26-3.36 (m, 1H), 4.84 (br dd, 1H), 7.55 (t, 1H), 7.93 (t, 1H), 7.94 (d, 1H), 8.24 (dd, 1H), 8.28 (dd, 1H), 8.46 (d, 1H), 8.52 (dd, 1H), 9.05 (d, 1H).

### Example 765

### 5-{[(3R)-2-oxoazepan-3-yl]amino}-2-(1H-pyrazol-4-yl)[1,2,4]triazolo[1,5-c]quinazoline-7-carbonitrile

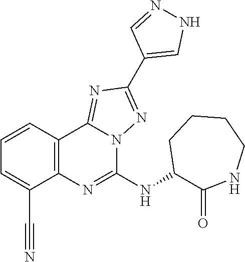

A mixture of 5-chloro-2-[1-(difluoromethyl)-1H-pyrazol-4-yl][1,2,4]triazolo[1,5-c]quinazoline-7-carbonitrile and 5-chloro-2-(1H-pyrazol-4-yl)[1,2,4]triazolo[1,5-c]quinazoline-7-carbonitrile (125 mg, about 362 μmol), (3R)-3-aminoazepan-2-one-hydrogen chloride (1/1) (89.3 mg, 542 μmol) and N,N-diisopropylethylamine (190 μL, 1.1 mmol) were stirred in DMSO (3.4 mL) for 90 minutes at 60° C. The reaction mixture was then cooled to rt and diluted with water. The solid was filtered, washed with water, dried under reduced pressure and purified by HPLC to obtain 10.4 mg (95% purity, 7% yield) of the title product and 12.2 mg (95% purity, 7% yield) of 2-[1-(difluoromethyl)-1H-pyrazol-4-yl]-5-{[(3R)-2-oxoazepan-3-yl]amino}[1,2,4]triazolo[1,5-c]quinazoline-7-carbonitrile LC-MS (Method 2): Rₜ=0.96 min; MS (ESIpos): m/z=388 [M+H]⁺

¹H-NMR (400 MHz, DMSO-d₆): δ [ppm]=1.26-1.42 (m, 1H), 1.49-1.61 (m, 1H), 1.83-1.99 (m, 2H), 2.00-2.08 (m, 1H), 2.37-2.44 (m, 1H), 3.15-3.23 (m, 1H), 3.25-3.35 (m, 1H), 4.84 (br dd, 1H), 7.53 (t, 1H), 7.89 (d, 1H), 8.15 (br s, 1H), 8.22 (dd, 1H), 8.26 (dd, 1H), 8.46-8.58 (m, 2H), 13.39 (s, 1H).

### Example 766

### (3R)-3-{[2-(4-methoxyphenyl)-7-(methylsulfanyl)[1,2,4]triazolo[1,5-c]quinazolin-5-yl]amino}azepan-2-one

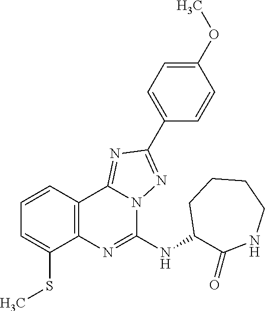

5-Chloro-2-(4-methoxyphenyl)-7-(methylsulfanyl)[1,2,4]triazolo[1,5-c]quinazoline (173 mg, 484 μmol), 1), (3R)-3-aminoazepan-2-one-hydrogen chloride (1/1) (87.7 mg, 533 μmol) and N,N-diisopropylethylamine (340 μL, 1.9 mmol) were stirred in DMSO (4.0 mL) for 2 h at 60° C. The mixture was cooled to rt and diluted with water. The solid was filtered, washed with water and dried under reduced pressure at 60° C. to give 207 mg (95% purity, 91% yield) of the title compound.

LC-MS (method 2): Rₜ=1.39 min; MS (ESIpos): m/z=449 [M+H]⁺

¹H-NMR (400 MHz, DMSO-d6): δ [ppm]=1.27-1.40 (m, 1H), 1.48-1.59 (m, 1H), 1.83-2.01 (m, 2H), 2.01-2.10 (m, 1H), 2.42 (br d, 1H), 3.14-3.24 (m, 1H), 3.26-3.32 (m, 1H), 3.86 (s, 3H), 4.78 (dd, 1H), 7.10-7.17 (m, 2H), 7.40-7.46 (m, 1H), 7.48-7.52 (m, 1H), 7.69 (d, 1H), 8.03 (dd, 1H), 8.18-8.27 (m, 3H).

The following examples were prepared similarly to example 766

| | Structure |
|---|---|
| | IUPAC-Name |
| | LC-MS (method): Retention time; Mass found |
| Example | ¹H-NMR |
| Example 767 | 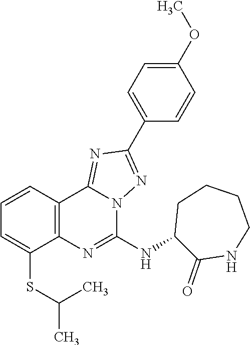 [SMILES: COc1ccc(-c2nc3c4cccc(SC(C)C)c4nc(N[C@@H]4CCCCNC4=O)n3n2)cc1] |
| | (3R)-3-({2-(4-methoxyphenyl)-7-[(propan-2- |
| | yl)sulfanyl][1,2,4]triazolo[1,5-c]quinazolin-5-yl}amino)azepan-2-one |
| | LC-MS (Method 2): Rₜ = 1.48 min; MS (ESIpos): m/z = 477 [M + H]⁺ |
| | ¹H-NMR (400 MHz, DMSO-d₆): δ [ppm] = 1.33 (d, 3H), 1.35 (d, 3H), 1.51- |
| | 1.62 (m, 1H), 1.85-1.97 (m, 2H), 2.02-2.10 (m, 1H), 2.38-2.45 (m, 1H), |
| | 3.15-3.25 (m, 1H), 3.76-3.84 (m, 1H), 3.86 (s, 3H), 4.79 (dd, 1H), 7.12- |
| | 7.17 (m, 2H), 7.41 (t, 1H), 7.68 (dd, 1H), 7.75 (d, 1H), 8.10 (dd, 1H), 8.20- |
| | 8.26 (m, 3H). |
| Example 768 | 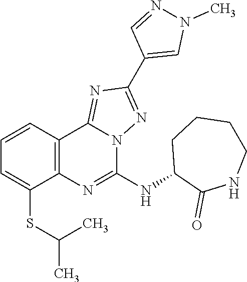 [SMILES: CC(C)Sc1cccc2c1nc(N[C@@H]1CCCCNC1=O)n1nc(-c3cnn(C)c3)nc21] |
| | (3R)-3-({2-(1-methyl-1H-pyrazol-4-yl)-7-[(propan-2- |
| | yl)sulfanoyl][1,2,4]triazolo[1,5-c]quinazolin-5-yl}amino)azepan-2-one |
| | LC-MS (Method 2): Rₜ = 1.23 min; MS (ESIpos): m/z = 451 [M + H]⁺ |
| | ¹H-NMR (400 MHz, DMSO-d₆): δ [ppm] = 1.33 (d, 3H), 1.34 (d, 3H), 1.46- |
| | 1.61 (m, 1H), 1.82-1.98 (m, 2H), 2.01-2.11 (m, 1H), 2.35-2.44 (m, 1H), |
| | 3.13-3.25 (m, 1H), 3.75-3.85 (m, 1H), 3.95 (s, 3H), 4.77 (br dd, 1H), 7.39 |
| | (t, 1H), 7.60-7.69 (m, 2H), 8.05 (dd, 1H), 8.07 (s, 1H), 8.23 (dd, 1H), 8.49 |
| | (s, 1H). |
| Example 769 | 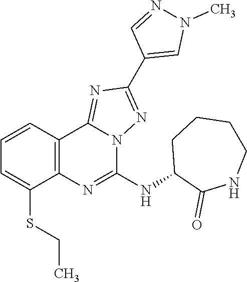 [SMILES: CCSc1cccc2c1nc(N[C@@H]1CCCCNC1=O)n1nc(-c3cnn(C)c3)nc21] |
| | (3R)-3-{[7-(ethylsulfanyl)-2-(1-methyl-1H-pyrazol-4-yl)[1,2,4]triazolo[1,5- |
| | c]quinazolin-5-yl]amino}azepan-2-one |
| | LC-MS (Method 2): Rₜ = 1.17 min; MS (ESIpos): m/z = 437 [M + H]⁺ |
| | ¹H-NMR (400 MHz, DMSO-d₆): δ [ppm] = 1.29-1.41 (m, 4H), 1.45-1.60 (m, |
| | 1H), 1.81-2.00 (m, 2H), 2.01-2.12 (m, 1H), 2.41 (br d, 1H), 3.02-3.10 (m, |
| | 2H), 3.13-3.24 (m, 1H), 3.95 (s, 3H), 4.76 (br dd, 1H), 7.40 (t, 1H), 7.56 |
| | (dd, 1H), 7.60 (d, 1H), 7.99 (dd, 1H), 8.07 (s, 1H), 8.24 (dd, 1H), 8.50 (s, |
| | 1H). |
| Example 770 | 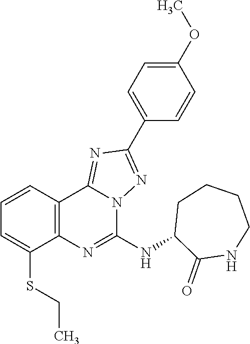 [SMILES: CCSc1cccc2c1nc(N[C@@H]1CCCCNC1=O)n1nc(-c3ccc(OC)cc3)nc21] |
| | (3R)-3-{[7-(ethylsulfanyl)-2-(4-methoxyphenyl)[1,2,4]triazolo[1,5- |
| | c]quinazolin-5-yl]amino}azepan-2-one |
| | LC-MS (Method 2): Rₜ = 1.43 min; MS (ESIpos): m/z = 463 [M + H]⁺ |
| | ¹H-NMR (400 MHz, DMSOd₆): δ [ppm] = 1.30-1.40 (m, 4H), 1.47-1.61 (m, |
| | 1H), 1.82-2.00 (m, 2H), 2.01-2.13 (m, 1H), 2.37-2.45 (m, 1H), 3.02-3.12 |
| | (m, 2H), 3.14-3.25 (m, 1H), 3.86 (s, 3H), 4.78 (dd, 1H), 7.12-7.16 (m, 2H), |
| | 7.41 (t, 1H), 7.58 (dd, 1H), 7.71 (d, 1H), 8.04 (dd, 1H), 8.05-8.05 (m, 1H), |
| | 8.19-8.27 (m, 3H). |
| Example 771 | 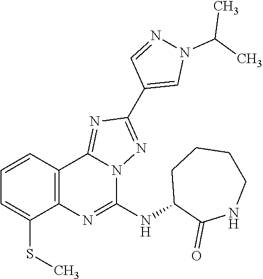 [SMILES: CSc1cccc2c1nc(N[C@@H]1CCCCNC1=O)n1nc(-c3cnn(C(C)C)c3)nc21] |
| | (3R)-3-({7-(methylsulfanyl)-2-[1-(propan-2-yl)-1H-pyrazol-4- |
| | yl][1,2,4]triazolo[1,5-c]quinazolin-5-yl}amino)azepan-2-one |
| | LC-MS (Method 2): Rₜ = 1.25 min; MS (ESIpos): m/z = 451 [M + H]⁺ |
| | ¹H-NMR (400 MHz, DMSO-d₆): δ [ppm] = 1.27-1.43 (m, 1H), 1.49-1.57 (m, |
| | 7H), 1.82-1.99 (m, 2H), 2.00-2.10 (m, 1H), 2.41 (br d, 1H), 3.15-3.25 (m, |
| | 1H), 3.25-3.31 (m, 1H), 4.64 (spt, 1H), 4.77 (dd, 1H), 7.39-7.46 (m, 1H), |
| | 7.47-7.54 (m, 1H), 7.59 (d, 1H), 7.99 (dd, 1H), 8.09 (s, 1H), 8.24 (dd, 1H), |
| | 8.54 (s, 1H)., |
| Example 772 | 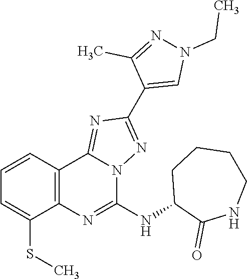 [SMILES: CCn1cc(-c2nc3c4cccc(SC)c4nc(N[C@@H]4CCCCNC4=O)n3n2)c(C)n1] |
| | (3R)-3-{[2-(1-ethyl-3-methyl-1H-pyrazol-4-yl)-7- |
| | (methylsulfanyl)[1,2,4]triazolo[1,5-c]quinazolin-5-yl]amino}azepan-2- |
| | one |
| | LC-MS (Method 2): Rₜ = 1.25 min; MS (ESIpos): m/z = 451 [M + H]⁺ |
| | ¹H-NMR (400 MHz, DMSO-d₆): δ [ppm] = 1.33 (br d, 1H), 1.42 (t, 3H), 1.47- |
| | 1.58 (m, 1H), 1.84-2.02 (m, 2H), 2.04 (br s, 1H), 2.39-2.47 (m, 1H), 2.59 |
| | (s, 3H), 3.15-3.25 (m, 1H), 3.25-3.32 (m, 1H), 4.16 (q, 2H), 4.76 (dd, 1H), |
| | 7.38-7.46 (m, 1H), 7.47-7.52 (m, 1H), 7.63 (d, 1H), 7.97 (dd, 1H), 8.22 |
| | (dd, 1H), 8.42 (s, 1H). |

### Example 773

### (2R)-2-{[7-bromo-2-(4-methoxyphenyl)[1,2,4]triazolo[1,5-c]quinazolin-5-yl]amino}-1-(morpholin-4-yl)propan-1-one

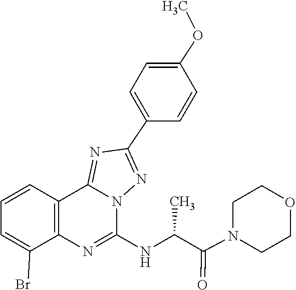 [SMILES: COc1ccc(-c2nc3c4cccc(Br)c4nc(N[C@H](C)C(=O)N4CCOCC4)n3n2)cc1]

7-Bromo-5-chloro-2-(4-methoxyphenyl)[1,2,4]triazolo[1,5-c]quinazoline (60.0 mg, 154 μmol) was suspended in DMSO (0.96 mL). (2R)-2-Amino-1-(morpholin-4-yl)propan-1-one-hydrogen chloride (1/1) (45.0 mg, 231 μmol) and N,N-diisopropylethylamine (110 μL, 620 μmol) were added. It was stirred for 1 h at 60° C. The reaction mixture was allowed to cool down and the solid was filtered off, washed with DMSO (2×0.5 mL) and three times with water. It was dried under vacuum at 50° C. yielding 48.5 mg (62%) of the title product.

LC-MS (Method 2): Rₜ=1.38 min; MS (ESIpos): m/z=511 [m+H]⁺

¹H-NMR (400 MHz, DMSO-d₆): δ [ppm]=1.55 (d, 3H), 3.40-3.49 (m, 1H), 3.53-3.79 (m, 7H), 3.86 (s, 3H), 5.21 (quin, 1H), 7.13-7.18 (m, 2H), 7.33 (t, 1H), 8.05 (dd, 1H), 8.23-8.31 (m, 4H).

The following examples were prepared analogously to example 773.

| | Structure |
|---|---|
| | IUPAC-Name |
| | LC-MS (method): Retention time; Mass found |
| Example | ¹H-NMR |
| Example 774 | 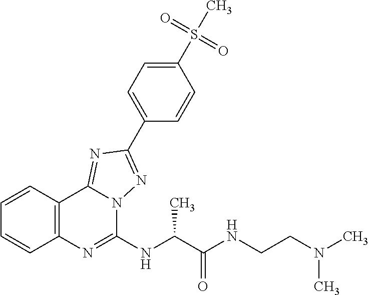 [SMILES: C[C@@H](Nc1nc2ccccc2c2nc(-c3ccc(S(C)(=O)=O)cc3)nn12)C(=O)NCCN(C)C] |
| | N-[2-(dimethylamino)ethyl]-N²-{2-[4- |
| | (methanesulfonyl)phenyl][1,2,4]triazolo[1,5-c]quinazolin-5-yl}-D- |
| | alaninamide |
| | LC-MS (Method 2): Rₜ = 1.41 min; MS (ESIpos): m/z = 482 [M + H]⁺ |
| | ¹H-NMR (400 MHz, DMSO-d₆): δ [ppm] = 1.53 (d, 3H), 2.11 (s, 6H), 2.25-2.32 |
| | (m, 2H), 3.12-3.27 (m, 2H), 3.32 (s, 3H), 4.78 (quin, 1H), 7.46 (ddd, 1H), 7.64 |
| | (d, 1H), 7.73-7.78 (m, 1H), 7.94 (d, 1H), 8.14 (br t, 1H), 8.16-8.20 (m, 2H), |
| | 8.33 (dd, 1H), 8.55-8.58 (m, 2H). |
| Example 775 | 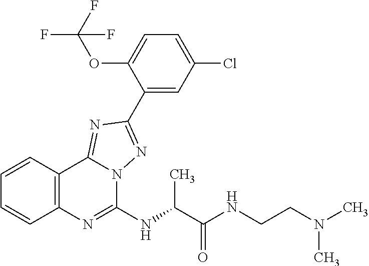 [SMILES: C[C@@H](Nc1nc2ccccc2c2nc(-c3cc(Cl)ccc3OC(F)(F)F)nn12)C(=O)NCCN(C)C] |
| | N²-{2-[5-chloro-2-(trifluoromethoxy)phenyl][1,2,4]triazolo[1,5- |
| | c]quinazolin-5-yl}-N-[2-(dimethylamino)ethyl]-D-alaninamide |
| | LC-MS (Method 2): Rₜ = 1.68 min; MS (ESIpos): m/z = 522 [M + H]⁺ |
| | ¹H-NMR (400 MHz, DMSO-d₆): δ [ppm] = 1.51 (d, 3H), 2.12 (s, 6H), 2.30 (t, 2H), |
| | 3.13-3.27 (m, 2H), 4.78 (quin, 1H), 7.48 (ddd, 1H), 7.64-7.70 (m, 2H), 7.71 |
| | (d, 1H), 7.74-7.82 (m, 2H), 8.17 (t, 1H), 8.28 (dd, 1H), 8.39 (d, 1H). |
| Example 776 | 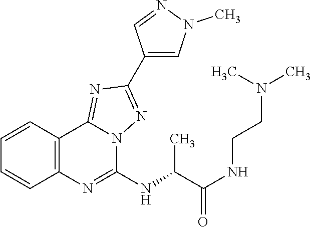 [SMILES: C[C@@H](Nc1nc2ccccc2c2nc(-c3cnn(C)c3)nn12)C(=O)NCCN(C)C] |
| | N-[2-(dimethylamino)ethyl]-N²-[2-(1-methyl-1H-pyrazol-4- |
| | yl)[1,2,4]triazolo[1,5-c]quinazolin-5-yl]-D-alaninamide |
| | LC-MS (Method 2): Rₜ = 0.89 min; MS (ESIpos): m/z = 408 [M + H]⁺ |
| | ¹H-NMR (400 MHz, DMSO-d₆): δ [ppm] = 1.50 (d, 3H), 2.10 (s, 6H), 2.24-2.31 |
| | (m, 2H), 3.11-3.26 (m, 2H), 3.96 (s, 3H), 4.76 (quin, 1H), 7.43 (ddd, 1H), 7.59- |
| | 7.65 (m, 2H), 7.71 (ddd, 1H), 8.07 (d, 1H), 8.14 (t, 1H), 8.23 (dd, 1H), 8.47 |
| | (s, 1H). |
| Example 777 | 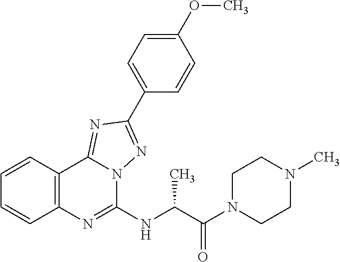 [SMILES: COc1ccc(-c2nc3c4ccccc4nc(N[C@H](C)C(=O)N4CCN(C)CC4)n3n2)cc1] |
| | (2R)-2-{[2-(4-methoxyphenyl)[1,2,4]triazolo[1,5-c]quinazolin-5-yl]amino}- |
| | 1-(4-methylpiperazin-1-yl)propan-1-one |
| | LC-MS (Method 2): Rₜ = 0.89 min; MS (ESIpos): m/z = 408 [M + H]⁺ |
| | ¹H-NMR (400 MHz, DMSO-d₆): δ [ppm] = 1.50 (d, 3H), 2.19-2.28 (m, 4H), 2.31- |
| | 2.39 (m, 1H), 2.41-2.47 (m, 2H), 3.39-3.47 (m, 1H), 3.56-3.66 (m, 2H), |
| | 3.69-3.79 (m, 1H), 3.86 (s, 3H), 5.19 (quin, 1H), 7.12-7.18 (m, 2H), 7.45 |
| | (ddd, 1H), 7.64 (d, 1H), 7.74 (ddd, 1H), 7.85 (d, 1H), 8.22-8.27 (m, 2H), 8.29 |
| | (dd, 1H). |
| Example 778 | 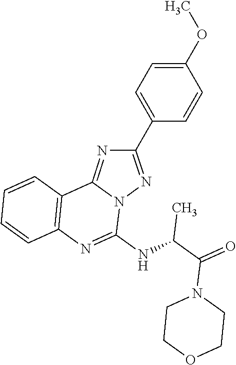 [SMILES: COc1ccc(-c2nc3c4ccccc4nc(N[C@H](C)C(=O)N4CCOCC4)n3n2)cc1] |
| | (2R)-2-{[2-(4-methoxyphenyl)[1,2,4]triazolo[1,5-c]quinazolin-5-yl]amino}- |
| | 1-(morpholin-4-yl)propan-1-one |
| | LC-MS (Method 2): Rₜ = 1.20 min; MS (ESIpos): m/z = 433 [M + H]⁺ |
| | ¹H-NMR (400 MHz, DMSO-d₆): δ [ppm] = 1.51 (d, 3H), 3.41-3.50 (m, 1H), 3.52- |
| | 3.80 (m, 7H), 3.86 (s, 3H), 5.19 (quin, 1H), 7.13-7.17 (m, 2H), 7.42-7.47 |
| | (m, 1H), 7.64 (d, 1H), 7.73 (ddd, 1H), 7.90 (d, 1H), 8.23-8.27 (m, 2H), 8.29 |
| | (dd, 1H). |
| Example 779 | 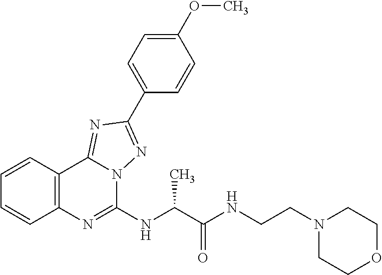 [SMILES: COc1ccc(-c2nc3c4ccccc4nc(N[C@H](C)C(=O)NCCN4CCOCC4)n3n2)cc1] |
| | N²-[2-(4-methoxyphenyl)[1,2,4]triazolo[1,5-c]quinazolin-5-yl]-N-[2- |
| | (morpholin-4-yl)ethyl]-D-alaninamide |
| | LC-MS (Method 2): Rₜ = 1.13 min; MS (ESIpos): m/z = 476 [M + H]⁺ |
| | ¹H-NMR (400 MHz, DMSO-d₆): δ [ppm] = 1.53 (d ,3H), 2.26-2.38 (m, 6H), 3.22 |
| | (q, 2H), 3.46 (t, 4H), 3.86 (s, 3H), 4.76 (quin, 1H), 7.12-7.17 (m, 2H), 7.44 |
| | (ddd, 1H), 7.62 (d, 1H), 7.72 (ddd, 1H), 7.80 (d, 1H), 8.10 (t, 1H), 8.23-8.31 |
| | (m, 3H). |
| Example 780 | 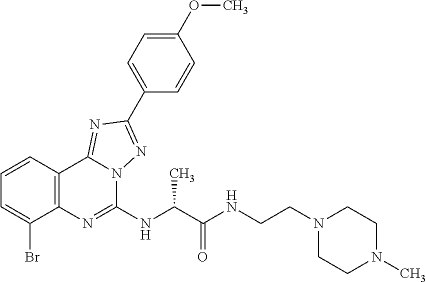 [SMILES: COc1ccc(-c2nc3c4cccc(Br)c4nc(N[C@H](C)C(=O)NCCN4CCN(C)CC4)n3n2)cc1] |
| | N²-[7-bromo-2-(4-methoxyphenyl)[1,2,4]triazolo[1,5-c]quinazolin-5-yl]-N- |
| | [2-(4-methylpiperazin-1-yl)ethyl]-D-alaninamide |
| | LC-MS (Method 2): Rₜ = 1.29 min; MS (ESIpos): m/z = 567 [M + H]⁺ |
| | ¹H-NMR (400 MHz, DMSO-d₆): δ [ppm] = 1.57 (d, 3H), 1.91-2.39 (m, 13H), |
| | 3.21 (q, 2H), 3.86 (s, 3H), 4.78 (quin, 1H), 7.13-7.18 (m, 2H), 7.33 (t, 1H), |
| | 8.01-8.08 (m, 2H), 8.20 (d, 1H), 8.23-8.28 (m, 2H), 8.30 (dd, 1H). |
| Example 781 | 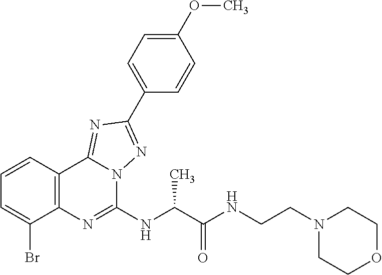 [SMILES: COc1ccc(-c2nc3c4cccc(Br)c4nc(N[C@H](C)C(=O)NCCN4CCOCC4)n3n2)cc1] |
| | N²-[7-bromo-2-(4-methoxyphenyl)[1,2,4]triazolo[1,5-c]quinazolin-5-yl]-N- |
| | [2-(morpholin-4-yl)ethyl]-D-alaninamide |
| | LC-MS (Method 2): Rₜ = 1.29 min; MS (ESIpos): m/z = 554 [M + H]⁺ |
| | ¹H-NMR (400 MHz, DMSO-d₆): δ [ppm] = 1.58 (d, 3H), 2.23-2.40 (m, 6H), 3.23 |
| | (br q, 2H), 3.39-3.47 (m, 4H), 3.86 (s, 3H), 4.79 (quin, 1H), 7.13-7.18 (m, |
| | 2H), 7.33 (t, 1H), 8.06 (dd, 1H), 8.09-8.18 (m, 2H), 8.23-8.28 (m, 2H), 8.30 |
| | (dd, 1H). |
| Example 782 | 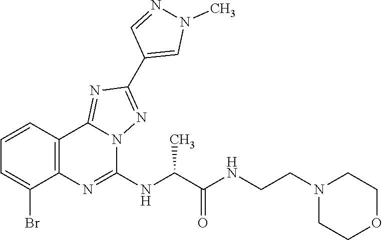 [SMILES: C[C@@H](Nc1nc2c(Br)cccc2c2nc(-c3cnn(C)c3)nn12)C(=O)NCCN1CCOCC1] |
| | N²-[7-bromo-2-(1-methyl-1H-pyrazol-4-yl)[1,2,4]triazolo[1,5-c]quinazolin- |
| | 5-yl]-N-[2-(morpholin-4-yl)ethyl]-D-alaninamide |
| | LC-MS (Method 2): Rₜ = 1.01 min; MS (ESIpos): m/z = 528 [M + H]⁺ |
| | ¹H-NMR (400 MHz, DMSO-d₆): δ [ppm] = 1.56 (d, 3H), 2.26-2.37 (m, 6H), 3.17- |
| | 3.28 (m, 2H), 3.43 (t, 4H), 3.96 (s, 3H), 4.77 (quin, 1H), 7.32 (t, 1H), 8.02- |
| | 8.06 (m, 2H), 8.07 (d, 1H), 8.12 (t, 1H), 8.24 (dd, 1H), 8.46 (s, 1H). |
| Example 783 | 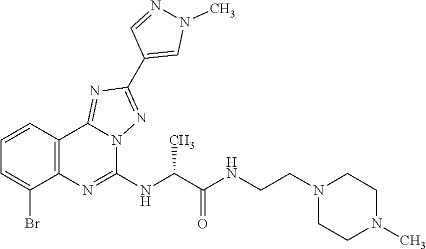 [SMILES: C[C@@H](Nc1nc2c(Br)cccc2c2nc(-c3cnn(C)c3)nn12)C(=O)NCCN1CCN(C)CC1] |
| | N²-[7-bromo-2-(1-methyl-1H-pyrazol-4-yl)[1,2,4]triazolo[1,5-c]quinazolin- |
| | 5-yl]-N-[2-(4-methylpiperazin-1-yl)ethyl]-D-alaninamide |
| | LC-MS (Method 2): Rₜ = 0.90 min; MS (ESIpos): m/z = 540 [M + H]⁺ |
| | ¹H-NMR (400 MHz, DMSO-d₆): δ [ppm] = 1.55 (d, 3H), 1.91-2.39 (m, 13H), |
| | 3.20 (q, 2H), 3.96 (s, 3H), 4.77 (quin, 1H), 7.32 (t, 1H), 8.01-8.10 (m, 4H), |
| | 8.24 (dd, 1H), 8.46 (s, 1H). |
| Example 784 | 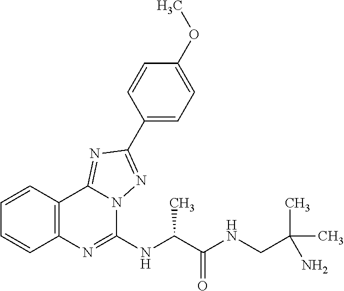 [SMILES: COc1ccc(-c2nc3c4ccccc4nc(N[C@H](C)C(=O)NCC(C)(C)N)n3n2)cc1] |
| | N-(2-amino-2-methylpropyl)-N²-[2-(4-methoxyphenyl)[1,2,4]triazolo[1,5- |
| | c]quinazolin-5-yl]-D-alaninamide |
| | LC-MS (Method 2): Rₜ = 1.13 min; MS (ESIpos): m/z = 434 [M + H]⁺ |
| | ¹H-NMR (400 MHz, DMSO-d₆): δ [ppm] = 0.95 (s, 3H), 0.95 (s, 3H), 1.46 (br s, |
| | 2H), 1.55 (d, 3H), 2.96-3.07 (m, 2H), 3.86 (s, 3H), 4.77-4.86 (m, 1H), 7.12- |
| | 7.17 (m, 2H), 7.44 (ddd, 1H), 7.61 (d, 1H), 7.72 (ddd, 1H), 7.77 (br s, 1H), 8.10 |
| | (t, 1H), 8.23-8.27 (m, 2H), 8.29 (dd, 1H). |
| Example 785 | 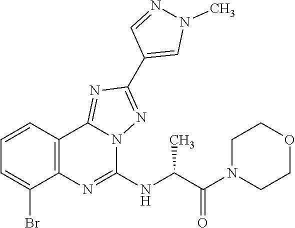 [SMILES: C[C@@H](Nc1nc2c(Br)cccc2c2nc(-c3cnn(C)c3)nn12)C(=O)N1CCOCC1] |
| | (2R)-2-{[7-bromo-2-(1-methyl-1H-pyrazol-4-yl)[1,2,4]triazolo[1,5- |
| | c]quinazolin-5-yl]amino}-1-(morpholin-4-yl)propan-1-one |
| | LC-MS (Method 2): Rₜ = 1.04 min; MS (ESIpos): m/z = 485 [M + H]⁺ |
| | ¹H-NMR (400 MHz, DMSO-d₆): δ [ppm] = 1.54 (d, 3H), 3.41-3.49 (m, 1H), 3.52- |
| | 3.79 (m, 7H), 3.96 (s, 3H), 5.19 (quin, 1H), 7.32 (t, 1H), 8.05 (dd, 1H), 8.07 |
| | (s, 1H), 8.12 (d, 1H), 8.24 (dd, 1H), 8.46 (s, 1H). |
| Example 786 | 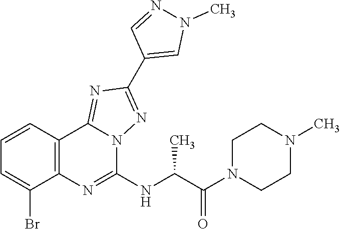 [SMILES: C[C@@H](Nc1nc2c(Br)cccc2c2nc(-c3cnn(C)c3)nn12)C(=O)N1CCN(C)CC1] |
| | (2R)-2-{[7-bromo-2-(1-methyl-1H-pyrazol-4-yl)[1,2,4]triazolo[1,5- |
| | c]quinazolin-5-yl]amino}-1-(4-methylpiperazin-1-yl)propan-1-one |
| | LC-MS (Method 2): Rₜ = 1.03 min; MS (ESIpos): m/z = 498 [M + H]⁺ |
| | ¹H-NMR (400 MHz, DMSO-d₆): δ [ppm] = 1.53 (d, 3H), 2.20 (s, 3H), 2.22-2.29 |
| | (m, 1H), 2.30-2.40 (m, 2H), 3.42-3.51 (m, 1H), 3.52-3.59 (m, 1H), 3.61- |
| | 3.73 (m, 2H), 3.96 (s, 3H), 5.20 (quin, 1H), 7.32 (t, 1H), 8.03-8.09 (m, 3H), |
| | 8.24 (dd, 1H), 8.46 (s, 1H). |
| Example 787 | 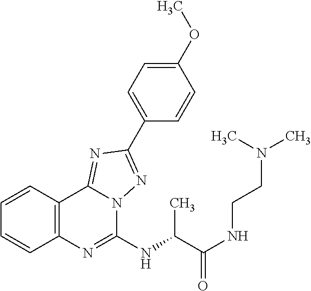 [SMILES: COc1ccc(-c2nc3c4ccccc4nc(N[C@H](C)C(=O)NCCN(C)C)n3n2)cc1] |
| | N-[2-(dimethylamino)ethyl]-N²-[2-(4-methoxyphenyl)[1,2,4]triazolo[1,5- |
| | c]quinazolin-5-yl]-D-alaninamide |
| | LC-MS (Method 2): Rₜ = 1.18 min; MS (ESIpos): m/z = 434 [M + H]⁺ |
| | ¹H-NMR (400 MHz, DMSO-d₆): δ [ppm] = 1.52 (d, 3H), 2.10 (s, 6H), 2.28 (td, |
| | 2H), 3.12-3.26 (m, 2H), 3.86 (s, 3H), 4.77 (quin, 1H), 7.13-7.17 (m, 2H), |
| | 7.42-7.47 (m, 1H), 7.60-7.63 (m, 1H), 7.70-7.75 (m, 1H), 7.77 (d, 1H), 8.14 |
| | (t, 1H), 8.23-8.31 (m, 3H). |
| Eaxmple 788 | 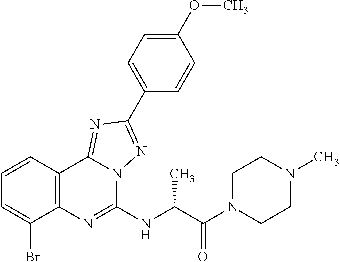 [SMILES: COc1ccc(-c2nc3c4cccc(Br)c4nc(N[C@H](C)C(=O)N4CCN(C)CC4)n3n2)cc1] |
| | (2R)-2-{[7-bromo-2-(4-methoxyphenyl)[1,2,4]triazolo[1,5-c]quinazolin-5- |
| | yl]amino}-1-(4-methylpiperazin-1-yl)propan-1-one |
| | LC-MS (Method 2): Rₜ = 0.89 min; MS (ESIpos): m/z = 408 [M + H]⁺ |
| | ¹H-NMR (400 MHz, DMSO-d₆): δ [ppm] = 1.54 (d, 3H), 2.18-2.30 (m, 4H), 2.31- |
| | 2.41 (m, 2H), 3.44-3.60 (m, 2H), 3.64-3.76 (m, 2H), 3.86 (s, 3H), 5.23 |
| | (quin, 1H), 7.12-7.17 (m, 2H), 7.32 (t, 1H), 8.04 (dd, 1H), 8.21-8.26 (m, 3H), |
| | 8.28 (dd, 1H). |
| Example 789 | 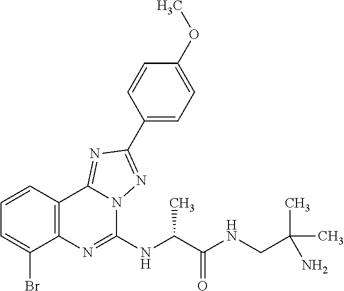 [SMILES: COc1ccc(-c2nc3c4cccc(Br)c4nc(N[C@H](C)C(=O)NCC(C)(C)N)n3n2)cc1] |
| | N-(2-amino-2-methylpropyl)-N²-[7-bromo-2-(4- |
| | methoxyphenyl)[1,2,4]triazolo[1,5-c]quinazolin-5-yl]-D-alaninamide |
| | LC-MS (Method 2): Rₜ = 1.21 min; MS (ESIpos): m/z = 512 [M + H]⁺ |
| | ¹H-NMR (400 MHz, DMSO-d₆): δ [ppm] = 0.95 (s, 3H), 0.95 (s, 3H), 1.46 (br s, |
| | 2H), 1.61 (d, 3H), 3.03 (dd, 1H), 3.06 (dd, 1H), 3.86 (s, 3H), 4.88 (q, 1H), 7.12- |
| | 7.17 (m, 2H), 7.33 (t, 1H), 8.05 (dd, 1H), 8.13 (br t, 1H), 8.22-8.27 (m, 2H), |
| | 8.29 (dd, 1H). |
| Example 790 | 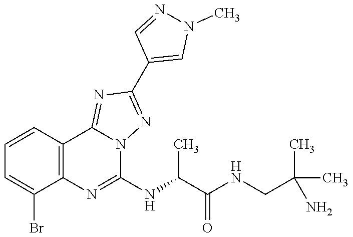 [SMILES: C[C@@H](Nc1nc2c(Br)cccc2c2nc(-c3cnn(C)c3)nn12)C(=O)NCC(C)(C)N] |
| | N-(2-amino-2-methylpropyl)-N²-[7-bromo-2-(1-methyl-1H-pyrazol-4- |
| | yl)[1,2,4]triazolo[1,5-c]quinazolin-5-yl]-D-alaninamide |
| | LC-MS (Method 2): Rₜ = 0.89 min; MS (ESIpos): m/z = 408 [M + H]⁺ |
| | ¹H-NMR (400 MHz, DMSO-d₆): δ [ppm] = 0.95 (s, 3H), 0.95 (s, 3H), 1.9 (d, |
| | 3H), 3.01 (dd, 1H), 3.05 (dd, 1H), 3.96 (s, 3H), 4.86 (q, 1H), 7.32 (t, 1H), 8.05 |
| | (dd, 1H), 8.07 (d, 1H), 8.13 (s, 1H), 8.24 (dd, 1H), 8.46 (s, 1H). three protons |
| | are missing |
| Example 791 | 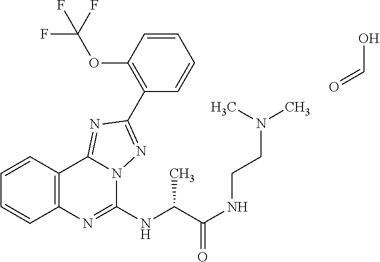 [SMILES: C[C@@H](Nc1nc2ccccc2c2nc(-c3ccccc3OC(F)(F)F)nn12)C(=O)NCCN(C)C ; O=CO] |
| | formic acid-N-[2-(dimethylamino)ethyl]-N²-{2-[2- |
| | (trifluoromethoxy)phenyl][1,2,4]triazolo[1,5-c]quinazolin-5-yl}-D- |
| | alaninamide (1/1) |
| | LC-MS (Method 1): Rt = 0.97 min; MS (ESIpos): m/z = 488 [M + H]⁺ |
| | ¹H-NMR (400 MHz, DMSO-d₆): δ [ppm] = 1.17 (d, 3H), 2.32 (s, 6H), 2.52-2.57 |
| | (m, 2H), 3.67-3.79 (m, 2H), 4.04 (q, 1H), 7.12 (br d, 1H), 7.15-7.20 (m, 1H), |
| | 7.39-7.44 (m, 1H), 7.47-7.51 (m, 1H), 7.51-7.62 (m, 3H), 8.16 (s, 1H), 8.19- |
| | 8.26 (m, 2H), 13.99 (br s, 1H). |
| Example 792 | 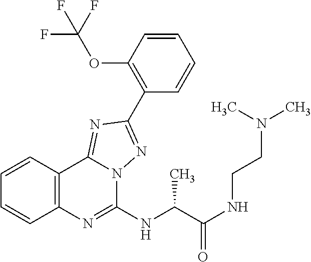 [SMILES: C[C@@H](Nc1nc2ccccc2c2nc(-c3ccccc3OC(F)(F)F)nn12)C(=O)NCCN(C)C] |
| | N-[2-(dimethylamino)ethyl]-N²-{2-[2- |
| | (trifluoromethoxy)phenyl][1,2,4]triazolo[1,5-c]quinazolin-5-yl}-D- |
| | alaninamide |
| | LC-MS (Method 1): Rt = 0.97 min; MS (ESIpos): m/z = 488 [M + H]⁺ |
| | ¹H-NMR (400 MHz, DMSO-d₆): δ [ppm] = 1.51 (d, 3H), 2.12 (s, 6H), 2.29 (t, 2H), |
| | 3.13-3.27 (m, 2H), 4.78 (quin, 1H), 7.47 (ddd, 1H), 7.59-7.68 (m, 4H), 7.70- |
| | 7.78 (m, 2H), 8.19 (t, 1H), 8.28 (dd, 1H), 8.35 (dd, 1H). |
| Example 793 | 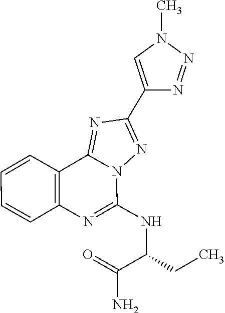 [SMILES: CC[C@@H](Nc1nc2ccccc2c2nc(-c3cn(C)nn3)nn12)C(N)=O] |
| | (2R)-2-{[2-(1-methyl-1H-1,2,3-triazol-4-yl)[1,2,4]triazolo[1,5-c]quinazolin- |
| | 5-yl]amino}butanamide |
| | LC-MS (Method 1): Rt = 0.79 min; MS (ESIpos): m/z = 352 [M + H]⁺ |
| | ¹H-NMR (400 MHz, DMSO-d₆): δ [ppm] = 0.95 (t, 3H), 1.87-2.00 (m, 1H), 2.02- |
| | 2.14 (m, 1H), 4.18 (s, 3H), 4.70 (td, 1H), 7.34 (s, 1H), 7.45 (ddd, 1H), 7.58 |
| | (d, 1H), 7.64 (d, 1H), 7.70-7.77 (m, 2H), 8.26 (dd, 1H), 8.85 (s, 1H) |
| Example 794 | 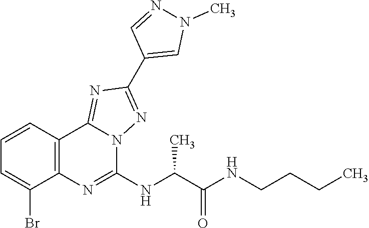 [SMILES: CCCCNC(=O)[C@@H](C)Nc1nc2c(Br)cccc2c2nc(-c3cnn(C)c3)nn12] |
| | N²-[7-bromo-2-(1-methyl-1H-pyrazol-4-yl)[1,2,4]triazolo[1,5-c]quinazolin- |
| | 5-yl]-N-butyl-D-alaninamide |
| | LC-MS (Method 1): Rt = 1.16 min; MS (ESIpos): m/z = 471 [M + H]⁺ |
| | ¹H-NMR (400 MHz, DMSO-d₆): δ [ppm] = 0.84 (t, 3H), 1.26 (sxt, 2H), 1.41 (quin, |
| | 2H), 1.56 (d, 3H), 3.11 (q, 2H), 3.96 (s, 3H), 4.77 (quin, 1H), 7.32 (t, 1H), 7.93 |
| | (d, 1H), 8.04 (dd, 1H), 8.07 (s, 1H), 8.17 (br t, 1H), 8.24 (dd, 1H), 8.46 (s, 1H). |
| Example 795 | 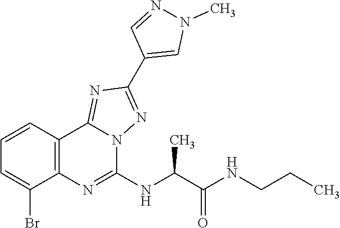 [SMILES: CCCNC(=O)[C@H](C)Nc1nc2c(Br)cccc2c2nc(-c3cnn(C)c3)nn12] |
| | N²-[7-bromo-2-(1-methyl-1H-pyrazol-4-yl)[1,2,4]triazolo[1,5-c]quinazolin- |
| | 5-yl]-N-propyl-L-alaninamide |
| | LC-MS (Method 1): Rt = 1.08 min; MS (ESIpos): m/z = 457 [M + H]⁺ |
| | ¹H-NMR (400 MHz, DMSO-d₆): δ [ppm] = 0.83 (t, 3H), 1.44 (sxt, 2H), 1.57 (d, |
| | 3H), 3.08 (q, 2H), 3.96 (s, 3H), 4.78 (quin, 1H), 7.32 (t, 1H), 7.93 (d, 1H), 8.04 |
| | (dd, 1H), 8.07 (d, 1H), 8.20 (t, 1H), 8.24 (dd, 1H), 8.46 (s, 1H). |
| Eample 796 | 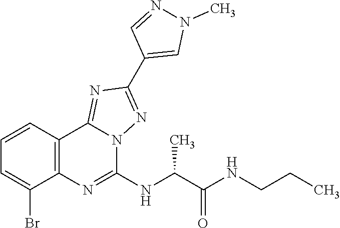 [SMILES: CCCNC(=O)[C@@H](C)Nc1nc2c(Br)cccc2c2nc(-c3cnn(C)c3)nn12] |
| | N²-[7-bromo-2-(1-methyl-1H-pyrazol-4-yl)[1,2,4]triazolo[1,5-c]quinazolin- |
| | 5-yl]-N-propyl-D-alaninamide |
| | LC-MS (Method 1): Rt = 1.08 min; MS (ESIpos): m/z = 457 [M + H]⁺ |
| | ¹H-NMR (400 MHz, DMSO-d₆): δ [ppm] = 0.83 (t, 3H), 1.44 (sxt, 2H), 1.57 (d, |
| | 3H), 3.08 (q, 2H), 3.96 (s, 3H), 4.78 (quin, 1H), 7.32 (t, 1H), 7.93 (d, 1H), 8.04 |
| | (dd, 1H), 8.07 (s, 1H), 8.20 (t, 1H), 8.24 (dd, 1H), 8.46 (s, 1H). |
| Example 797 | 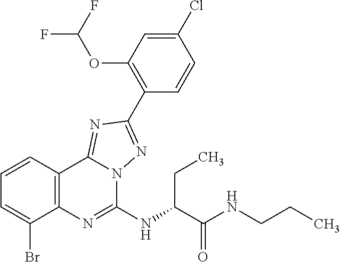 [SMILES: CCCNC(=O)[C@@H](CC)Nc1nc2c(Br)cccc2c2nc(-c3ccc(Cl)cc3OC(F)F)nn12] |
| | (2R)-2-({7-bromo-2-[4-chloro-2- |
| | (difluoromethoxy)phenyl][1,2,4]triazolo[1,5-c]quinazolin-5-yl}amino)-N- |
| | propylbutanamide |
| | LC-MS (Method 1): Rt = 1.60 min; MS (ESIpos): m/z = 567 [M + H]⁺ |
| | ¹H-NMR (400 MHz, DMSO-d₆): δ [ppm] = 0.85 (t, 3H), 0.96 (t, 3H), 1.45 (sxt, |
| | 2H), 1.89-2.01 (m, 1H), 2.07-2.19 (m, 1H), 3.02-3.18 (m, 2H), 4.70-4.77 |
| | (m, 1H), 7.36 (t, 1H), 7.39 (t, 1H), 7.56 (d, 1H), 7.62 (dd, 1H), 7.75 (d, 1H), |
| | 8.08 (dd, 1H), 8.25-8.32 (m, 3H). |
| Example 798 | 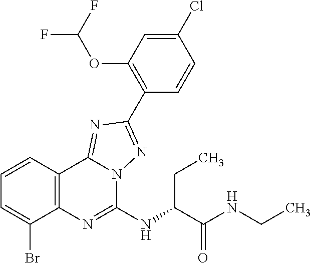 [SMILES: CCNC(=O)[C@@H](CC)Nc1nc2c(Br)cccc2c2nc(-c3ccc(Cl)cc3OC(F)F)nn12] |
| | (2R)-2-({7-bromo-2-[4-chloro-2- |
| | (difluoromethoxy)phenyl][1,2,4]triazolo[1,5-c]quinazolin-5-yl}amino)-N- |
| | ethylbutanamide |
| | LC-MS (Method 1): Rt = 1.47 min; MS (ESIpos): m/z = 553 [M + H]⁺ |
| | ¹H-NMR (400 MHz, DMSO-d₆): δ [ppm] = 0.96 (t, 3H), 1.05 (t, 3H), 1.88-2.00 |
| | (m, 1H), 2.06-2.18 (m, 1H), 3.16 (quin, 2H), 4.68-4.74 (m, 1H), 7.36 (t, 1H), |
| | 7.39 (t, 1H), 7.56 (d, 1H), 7.62 (dd, 1H), 7.76 (d, 1H), 8.08 (dd, 1H), 8.25-8.32 |
| | (m, 3H). |
| Example 799 | 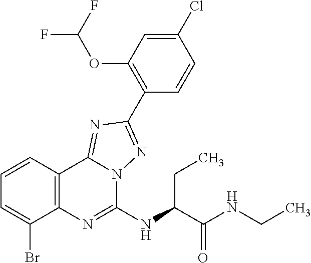 [SMILES: CCNC(=O)[C@H](CC)Nc1nc2c(Br)cccc2c2nc(-c3ccc(Cl)cc3OC(F)F)nn12] |
| | (2S)-2-({7-bromo-2-[4-chloro-2- |
| | (difluoromethoxy)phenyl][1,2,4]triazolo[1,5-c]quinazolin-5-yl}amino)-N- |
| | ethylbutanamide |
| | LC-MS (Method 1): Rt = 1.47 min; MS (ESIpos); m/z = 553[M + H]⁺ |
| | ¹H-NMR (400 MHz, DMSO-d₆): δ [ppm] = 0.96 (t, 3H), 1.05 (t, 3H), 1.88-2.00 |
| | (m, 1H), 2.07-2.18 (m, 1H), 3.12-3.20 (m, 2H), 4.67-4.74 (m, 1H), 7.36 (t, |
| | 1H), 7.39 (t, 1H), 7.56 (d, 1H), 7.62 (dd, 1H), 7.76 (br d, 1H), 8.08 (dd, 1H), |
| | 8.25-8.32 (m, 3H). |
| Example 800 | 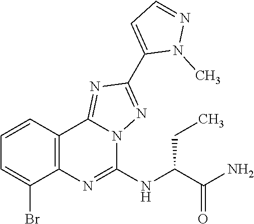 [SMILES: CC[C@@H](Nc1nc2c(Br)cccc2c2nc(-c3ccnn3C)nn12)C(N)=O] |
| | (2R)-2-{[7-bromo-2-(1-methyl-1H-pyrazol-5-yl)[1,2,4]triazolo[1,5- |
| | c]quinazolin-5-yl]amino}butanamide |
| | LC-MS (Method 1): Rt = 1.07 min; MS (ESIpos): m/z = 429 [M + H]⁺ |
| | ¹H-NMR (400 MHz, DMSO-d₆): δ [ppm] = 0.97 (t, 3H), 1.90-2.04 (m, 1H), 2.14- |
| | 2.26 (m, 1H), 4.36 (s, 3H), 4.74 (td, 1H), 7.04 (d, 1H), 7.31-7.37 (m, 2H), |
| | 7.62 (d, 1H), 7.72 (d, 1H), 7.97 (d, 1H), 8.08 (dd, 1H), 8.30 (dd, 1H). |
| Example 801 | 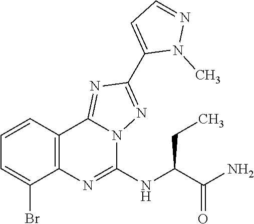 [SMILES: CC[C@H](Nc1nc2c(Br)cccc2c2nc(-c3ccnn3C)nn12)C(N)=O] |
| | (2S)-2-{[7-bromo-2-(1-methyl-1H-pyrazol-5-yl)[1,2,4]triazolo[1,5- |
| | c]quinazolin-5-yl]amino}butanamide |
| | LC-MS (Method 1): Rt = 1.07 min; MS (ESIpos): m/z = 429 [M + H]⁺ |
| | ¹H-NMR (400 MHz, DMSO-d₆): δ [ppm] = 0.97 (t, 3H), 1.91-2.03 (m, 1H), 2.14- |
| | 2.26 (m, 1H), 4.37 (s, 3H), 4.70-4.77 (m, 1H), 7.04 (d, 1H), 7.31-7.38 (m, |
| | 2H), 7.62 (d, 1H), 7.72 (s, 1H), 7.97 (br d, 1H), 8.08 (dd, 1H), 8.30 (dd, 1H). |
| Example 802 | 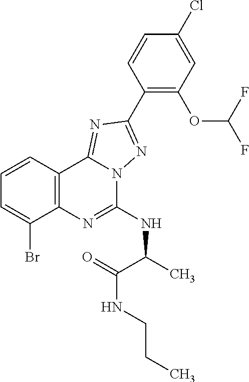 [SMILES: CCCNC(=O)[C@H](C)Nc1nc2c(Br)cccc2c2nc(-c3ccc(Cl)cc3OC(F)F)nn12] |
| | N²-{7-bromo-2-[4-chloro-2-(difluoromethoxy)phenyl][1,2,4]triazolo[1,5- |
| | c]quinazolin-5-yl}-N-propyl-L-alaninamide |
| | LC-MS (Method 1): Rt = 1.53 min; MS (ESIpos): m/z = 553 [M + H]⁺ |
| | ¹H-NMR (400 MHz, DMSO-d₆): δ [ppm] = 0.84 (t, 3H), 1.45 (sxt, 2H), 1.58 (d, |
| | 3H), 3.09 (q, 2H), 4.79 (quin, 1H), 7.36 (t, 1H), 7.40 (t, 1H), 7.56 (d, 1H), 7.62 |
| | (dd, 1H), 7.95 (d, 1H), 8.08 (dd, 1H), 8.20-8.27 (m, 1H), 8.28 (dd, 1H). |
| Example 803 | 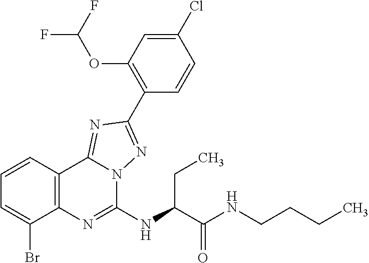 [SMILES: CCCCNC(=O)[C@H](CC)Nc1nc2c(Br)cccc2c2nc(-c3ccc(Cl)cc3OC(F)F)nn12] |
| | (2S)-2-({7-bromo-2-[4-chloro-2- |
| | (difluoromethoxy)phenyl][1,2,4]triazolo[1,5-c]quinazolin-5-yl}amino)-N- |
| | butylbutanamide |
| | LC-MS (Method 1): Rt = 1.63 min; MS (ESIpos): m/z = 581 [M + H]⁺ |
| | ¹H-NMR (400 MHz, DMSO-d₆): δ [ppm] = 0.85 (t, 3H), 0.96 (t, 3H), 1.22-1.34 |
| | (m, 2H), 1.38-1.46 (m, 2H), 1.88-2.01 (m, 1H), 2.06-2.18 (m, 1H), 3.05- |
| | 3.21 (m, 2H), 4.69-4.75 (m, 1H), 7.36 (t, 1H), 7.39 (t, 1H), 7.56 (d, 1H), 7.62 |
| | (dd, 1H), 7.75 (d, 1H), 8.09 (dd, 1H), 8.25-8.31 (m, 3H). |
| Example 804 | 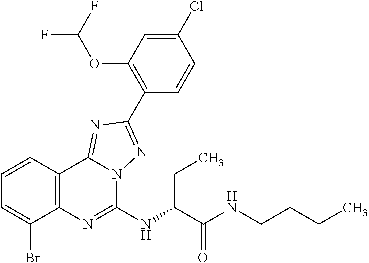 [SMILES: CCCCNC(=O)[C@@H](CC)Nc1nc2c(Br)cccc2c2nc(-c3ccc(Cl)cc3OC(F)F)nn12] |
| | (2R)-2-({7-bromo-2-[4-chloro-2- |
| | (difluoromethoxy)phenyl][1,2,4]triazolo[1,5-c]quianzolin-5-yl}amino)-N- |
| | butylbutanamide |
| | LC-MS (Method 1): Rt = 1.637 min; MS (ESIpos): m/z = 581 [M + H]⁺ |
| | ¹H-NMR (400 MHz, DMSO-d₆): δ [ppm] = 0.85 (t, 3H), 0.96 (t, 3H), 1.22-1.33 |
| | (m, 2H), 1.37-1.46 (m, 2H), 1.88-2.01 (m, 1H), 2.07-2.18 (m, 1H), 3.05- |
| | 3.22 (m, 2H), 4.69-4.76 (m, 1H), 7.36 (t, 1H), 7.39 (t, 1H), 7.56 (d, 1H), 7.62 |
| | (dd, 1H), 7.75 (br d, 1H), 8.08 (dd, 1H), 8.24-8.31 (m, 3H). |
| Example 805 | 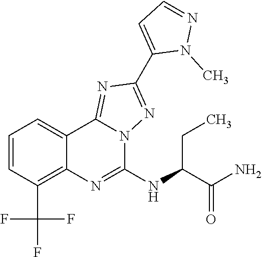 [SMILES: CC[C@H](Nc1nc2c(C(F)(F)F)cccc2c2nc(-c3ccnn3C)nn12)C(N)=O] |
| | (2S)-2-{[2-(1-methyl-1H-pyrazol-5-yl)-7- |
| | (trifluoromethyl)[1,2,4]triazolo[1,5-c]quinazolin-5-yl]amino}butanamide |
| | LC-MS (Method 2): Rₜ = 1.16 min; MS (ESIpos): m/z = 419 [M + H]⁺ |
| | ¹H-NMR (400 MHz, DMSO-d₆): δ [ppm] = 0.93 (t, 3H), 1.89-2.01 (m, 1H), 2.11- |
| | 2.22 (m, 1H), 4.37 (s, 3H), 4.70 (td, 1H), 7.05 (d, 1H), 7.36 (s, 1H), 7.55 (t, |
| | 1H), 7.62 (d, 1H), 7.72 (s, 1H), 8.00 (d, 1H), 8.11 (dd, 1H), 8.56 (dd, 1H). |
| Example 806 | 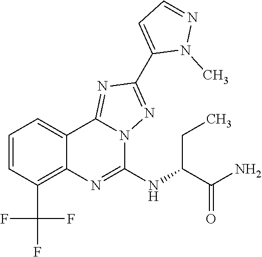 [SMILES: CC[C@@H](Nc1nc2c(C(F)(F)F)cccc2c2nc(-c3ccnn3C)nn12)C(N)=O] |
| | (2R)-2-{[2-(1-methyl-1H-pyrazol-5-yl)-7- |
| | (trifluoromethyl)[1,2,4]triazolo[1,5-c]quinazolin-5-yl]amino}butanamide |
| | LC-MS (Method 2): Rₜ = 1.16 min; MS (ESIpos): m/z = 419 [M + H]⁺ |
| | ¹H-NMR (400 MHz, DMSO-d₆): δ [ppm] = 0.93 (t, 3H), 1.88-2.02 (m, 1H), 2.11- |
| | 2.23 (m, 1H), 4.37 (s, 3H), 4.66-4.74 (m, 1H), 7.05 (d, 1H), 7.36 (s, 1H), |
| | 7.55 (t, 1H), 7.63 (d, 1H), 7.72 (s, 1H), 8.00 (br d, 1H), 8.09-8.13 (m, 1H), |
| | 8.55 (dd, 1H). |
| Example 807 | 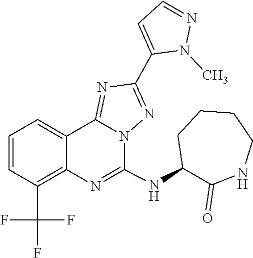 [SMILES: Cn1nccc1-c1nc2c3cccc(C(F)(F)F)c3nc(N[C@H]3CCCCNC3=O)n2n1] |
| | (3S)-3-{[2-(1-methyl-1H-pyrazol-5-yl)-7- |
| | (trifluoromethyl)[1,2,4]triazolo[1,5-c]quinazolin-5-yl]amino}azepan-2- |
| | one |
| | LC-MS (Method 2): Rₜ = 1.33 min; MS (ESIneg): m/z = 443 [M − H]⁻ |
| | ¹H-NMR (400 MHz, DMSO-d₆): δ [ppm] = 1.26-1.39 (m, 1H), 1.50-1.62 (m, |
| | 1H), 1.78-1.91 (m, 2H), 1.99-2.07 (m, 1H), 2.40 (br d, 1H), 3.15-3.30 (m, |
| | 2H), 4.35 (s, 3H), 4.77 (br dd, 1H), 7.05 (d, 1H), 7.57 (t, 1H), 7.62 (d, 1H), 7.95 |
| | (br d, 1H), 8.11-8.15 (m, 1H), 8.22-8.27 (m, 1H), 8.55 (dd, 1H). |
| Example 808 | 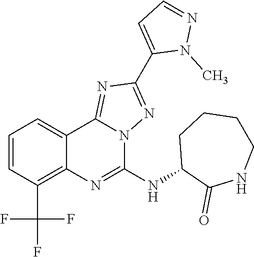 [SMILES: Cn1nccc1-c1nc2c3cccc(C(F)(F)F)c3nc(N[C@@H]3CCCCNC3=O)n2n1] |
| | (3R)-3-{[2-(1-methyl-1H-pyrazol-5-yl)-7- |
| | (trifluoromethyl)[1,2,4]triazolo[1,5-c]quinazolin-5-yl]amino}azepan-2- |
| | one |
| | LC-MS (Method 2): Rₜ = 1.34 min; MS (ESIneg): m/z = 443 [M − H]⁻ |
| | ¹H-NMR (400 MHz, DMSO-d₆): δ [ppm] = 1.26-1.39 (m, 1H), 1.50-1.62 (m, |
| | 1H), 1.78-1.92 (m, 2H), 1.98-2.08 (m, 1H), 2.40 (br d, 1H), 3.15-3.31 (m, |
| | 2H), 4.35 (s, 3H), 4.77 (dd, 1H), 7.05 (d, 1H), 7.57 (t, 1H), 7.63 (d, 1H), 7.95 |
| | (d, 1H), 8.11-8.15 (m, 1H), 8.22-8.27 (m, 1H), 8.56 (dd, 1H). |
| Example 809 | 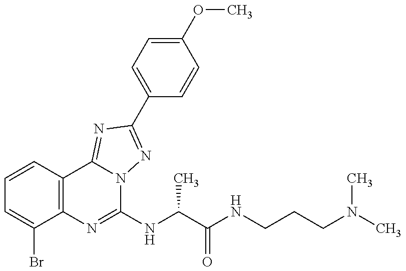 [SMILES: COc1ccc(-c2nc3c4cccc(Br)c4nc(N[C@H](C)C(=O)NCCCN(C)C)n3n2)cc1] |
| | N²-[7-bromo-2-(4-methoxyphenyl)[1,2,4]triazolo[1,5-c]quinazolin-5-yl]-N- |
| | [3-(dimethylamino)propyl]-D-alaninamide |
| | LC-MS (Method 1): Rₜ = 0.87 min; MS (ESIpos): m/z = 526 [M + H]⁺ |
| | ¹H-NMR (400 MHz, DMSO-d₆): δ [ppm] = 1.50-1.62 (m, 5H), 2.02 (s, 6H), 2.16 |
| | (t, 2H), 3.13 (q, 2H), 3.86 (s, 3H), 4.78 (quin, 1H), 7.13-7.18 (m, 2H), 7.33 (t, |
| | 1H), 8.05 (dd, 1H), 8.13 (d, 1H), 8.20 (br t, 1H), 8.23-8.27 (m, 2H), 8.30 (dd, |
| | 1H). |
| Example 810 | 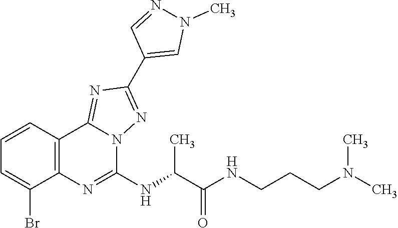 [SMILES: C[C@@H](Nc1nc2c(Br)cccc2c2nc(-c3cnn(C)c3)nn12)C(=O)NCCCN(C)C] |
| | N²-[7-bromo-2-(1-methyl-1H-pyrazol-4-yl)[1,2,4]triazolo[1,5-c]quinazolin- |
| | 5-yl]-N-[3-(dimethylamino)propyl]-D-alaninamide |
| | LC-MS (Method 2): Rₜ = 1.07 min; MS (ESIpos): m/z = 500 [M + H]⁺ |
| | ¹H-NMR (400 MHz, DMSO-d₆): δ [ppm] = 1.49-1.58 (m, 5H), 2.02 (s, 6H), 2.16 |
| | (t, 2H), 3.12 (q, 2H), 3.96 (s, 3H), 4.76 (quin, 1H), 7.32 (t, 1H), 8.00 (br d, 1H), |
| | 8.04 (dd, 1H), 8.07 (s, 1H), 8.20 (br t, 1H), 8.24 (dd, 1H), 8.46 (s, 1H). |
| Example 811 | 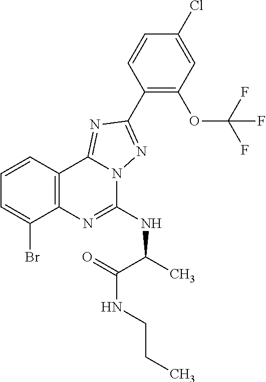 [SMILES: CCCNC(=O)[C@H](C)Nc1nc2c(Br)cccc2c2nc(-c3ccc(Cl)cc3OC(F)(F)F)nn12] |
| | N²-{7-bromo-2-[4-chloro-2-(trifluoromethoxy)phenyl][1,2,4]triazolo[1,5- |
| | c]quinazolin-5-yl}-N-propyl-L-alaninamide |
| | LC-MS (Method 2): Rₜ = 1.65 min; MS (ESIpos): m/z = 571 [M + H]⁺ |
| | ¹H-NMR (400 MHz, DMSO-d₆): δ [ppm] = 0.85 (t, 3H), 1.45 (sxt, 2H), 1.58 (d, |
| | 3H), 3.09 (q, 2H), 4.78 (quin, 1H), 7.36 (t, 1H), 7.76-7.81 (m, 2H), 7.90 (br d, |
| | 1H), 8.09 (dd, 1H), 8.24-8.30 (m, 2H), 8.40 (d, 1H). |
| Example 812 | 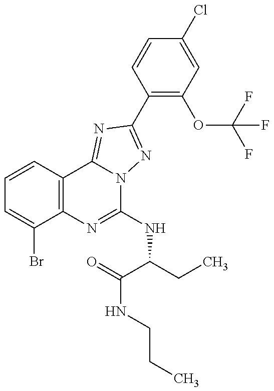 [SMILES: CCCNC(=O)[C@@H](CC)Nc1nc2c(Br)cccc2c2nc(-c3ccc(Cl)cc3OC(F)(F)F)nn12] |
| | (2R)-2-({7-bromo-2-[4-chloro-2- |
| | (trifluoromethoxy)phenyl][1,2,4]triazolo[1,5-c]quinazolin-5-yl}amino)-N- |
| | propylbutanamide |
| | LC-MS (Method 2): Rₜ = 1.71 min; MS (ESIpos): m/z = 585 [M + H]⁺ |
| | ¹H-NMR (400 MHz, DMSO-d₆): δ [ppm] = 0.85 (t, 3H), 0.95 (t, 3H), 1.45 (sxt, |
| | 2H), 1.88-2.01 (m, 1H), 2.10-2.22 (m, 1H), 3.02-3.19 (m, 2H), 4.72-4.78 |
| | (m, 1H), 7.37 (t, 1H), 7.70 (d, 1H), 7.76-7.82 (m, 2H), 8.09 (dd, 1H), 8.28 (dd, |
| | 1H), 8.33 (t, 1H), 8.42 (d, 1H). |
| Example 813 | 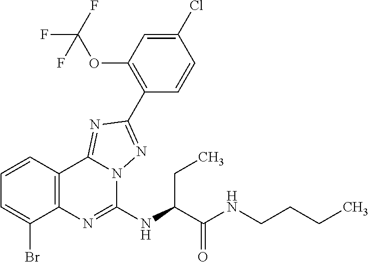 [SMILES: CCCCNC(=O)[C@H](CC)Nc1nc2c(Br)cccc2c2nc(-c3ccc(Cl)cc3OC(F)(F)F)nn12] |
| | (2S)-2-({7-bromo-2-[4-chloro-2- |
| | (trifluoromethoxy)phenyl][1,2,4]triazolo[1,5-c]quinazolin-5-yl}amino)-N- |
| | butylbutanamide |
| | LC-MS (Method 2): Rₜ = 1.75 min; MS (ESIpos): m/z = 599 [M + H]⁺ |
| | ¹H-NMR (400 MHz, DMSO-d₆): δ [ppm] = 0.86 (t, 3H), 0.94 (t, 3H), 1.21-1.34 |
| | (m, 2H), 1.38-1.47 (m, 2H), 1.87-2.00 (m, 1H), 2.09-2.21 (m, 1H), 3.06- |
| | 3.22 (m, 2H), 4.71-4.77 (m, 1H), 7.37 (t, 1H), 7.70 (d, 1H), 7.76-7.82 (m, |
| | 2H), 8.09 (dd, 1H), 8.26-8.33 (m, 2H), 8.42 (d, 1H). |
| Example 814 | 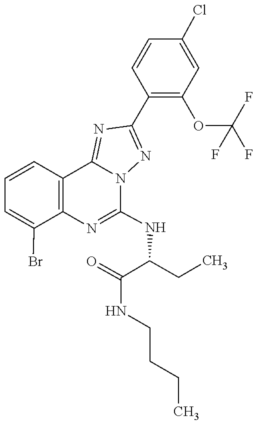 [SMILES: CCCCNC(=O)[C@@H](CC)Nc1nc2c(Br)cccc2c2nc(-c3ccc(Cl)cc3OC(F)(F)F)nn12] |
| | (2R)-2-({7-bromo-2-[4-chloro-2- |
| | (trifluoromethoxy)phenyl][1,2,4]triazolo[1,5-c]quinazolin-5-yl}amino)-N- |
| | butylbutanamide |
| | LC-MS (Method 2): Rₜ = 1.75 min; MS (ESIpos): m/z = 599 [M + H]⁺ |
| | ¹H-NMR (400 MHz, DMSO-d₆): δ [ppm] = 0.86 (t, 3H), 0.94 (t, 3H), 1.22-1.34 |
| | (m, 2H), 1.38-1.47 (m, 2H), 1.87-2.00 (m, 1H), 2.09-2.21 (m, 1H), 3.06- |
| | 3.23 (m, 2H), 4.71-4.78 (m, 1H), 7.37 (t, 1H), 7.70 (d, 1H), 7.76-7.83 (m, |
| | 2H), 8.09 (d, 1H), 8.26-8.33 (m, 2H), 8.42 (d, 1H). |
| Example 815 | 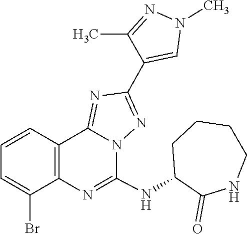 [SMILES: Cc1nn(C)cc1-c1nc2c3cccc(Br)c3nc(N[C@@H]3CCCCNC3=O)n2n1] |
| | (3R)-3-{[7-bromo-2-(1,3-dimethyl-1H-pyrazol-4-yl)[1,2,4]triazolo[1,5- |
| | c]quinazolin-5-yl]amino}azepan-2-one |
| | LC-MS (Method 2): Rₜ = 1.25 min; MS (ESIpos): m/z = 471 [M + H]⁺ |
| | ¹H-NMR (400 MHz, DMSO-d₆): δ [ppm] = 1.26-1.41 (m, 1H), 1.47-1.60 (m, |
| | 1H), 1.82-2.00 (m, 2H), 2.01-2.11 (m, 1H), 2.45 (br d, 1H), 2.58 (s, 3H), |
| | 3.13-3.25 (m, 1H), 3.29 (br d, 1H), 3.87 (s, 3H), 4.81 (br dd, 1H), 7.33 (t, |
| | 1H), 7.73 (d, 1H), 8.06 (dd, 1H) 8.20-8.27 (m, 2H), 8.38 (s, 1H). |
| Example 816 | 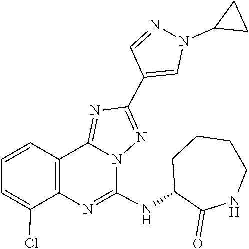 [SMILES: O=C1NCCCC[C@H]1Nc1nc2c(Cl)cccc2c2nc(-c3cnn(C4CC4)c3)nn12] |
| | (3R)-3-{[7-chloro-2-(1-cyclopropyl-1H-pyrazol-4-yl)[1,2,4]triazolo[1,5- |
| | c]quinazolin-5-yl]amino}azepan-2-one |
| | LC-MS (Method 2): Rₜ = 1.29 min; MS (ESIpos): m/z = 437 [M + H]⁺ |
| | ¹H-NMR (400 MHz, DMSO-d₆): δ [ppm] = 0.98-1.06 (m, 2H), 1.14-1.20 (m, |
| | 2H), 1.26-1.40 (m, 1H), 1.46-1.59 (m, 1H), 1.80-1.96 (m, 2H), 1.99-2.09 |
| | (m, 1H), 2.40 (br d, 1H), 3.15-3.22 (m, 1H), 3.88 (tt, 1H), 4.83 (dd, 1H), |
| | 7.40 (t, 1H), 7.70 (d, 1H), 7.89 (dd, 1H), 8.07 (d, 1H), 8.20 (dd, 1H), 8.24 |
| | (dd, 1H), 8.56 (s, 1H). |
| Example 817 | 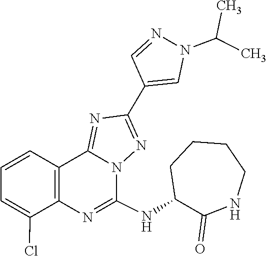 [SMILES: CC(C)n1cc(-c2nc3c4cccc(Cl)c4nc(N[C@@H]4CCCCNC4=O)n3n2)cn1] |
| | (3R)-3-({7-chloro-2-[1-(propan-2-yl)-1H-pyrazol-4-yl][1,2,4]triazolo[1,5- |
| | c]quinazolin-5-yl}amino)azepan-2-one |
| | LC-MS (Method 2): Rₜ = 1.32 min; MS (ESIpos): m/z = 439 [M + H]⁺ |
| | ¹H-NMR (400 MHz, DMSO-d₆): δ [ppm] = 1.25-1.41 (m, 1H), 1.49 (d, 6H), |
| | 1.52-1.60 (m, 1H), 1.80-1.96 (m, 2H), 1.99-2.10 (m, 1H), 2.41 (br d, 1H), |
| | 3.10-3.24 (m, 1H), 3.26-3.32 (m, 1H), 4.64 (spt, 1H), 4.83 (dd, 1H), 7.40 |
| | (t, 1H), 7.70 (d, 1H), 7.89 (dd, 1H), 8.09 (s, 1H), 8.20 (dd, 1H), 8.24 (dd, 1H), |
| | 8.54 (s, 1H). |
| Example 818 | 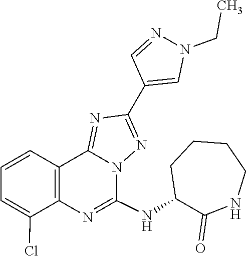 [SMILES: CCn1cc(-c2nc3c4cccc(Cl)c4nc(N[C@@H]4CCCCNC4=O)n3n2)cn1] |
| | (3R)-3-{[7-chloro-2-(1-ethyl-1H-pyrazol-4-yl)[1,2,4]triazolo[1,5- |
| | c]quinazolin-5-yl]amino}azepan-2-one |
| | LC-MS (Method 2): Rₜ = 1.26 min; MS (ESIpos): m/z = 425 [M + H]⁺ |
| | ¹H-NMR (400 MHz, DMSO-d₆): δ [ppm] = 1.26-1.39 (m, 1H), 1.44 (t, 3H), |
| | 1.48-1.61 (m, 1H), 1.81-1.96 (m, 2H), 1.98-2.12 (m, 1H), 2.41 (br d, 1H), |
| | 3.12-3.24 (m, 1H), 4.25 (q, 2H), 4.83 (dd, 1H), 7.40 (t, 1H), 7.71 (d, 1H), |
| | 7.89 (dd, 1H), 8.09 (s, 1H), 8.20 (dd, 1H), 8.24 (dd, 1H), 8.54 (s, 1H). |
| Example 819 | 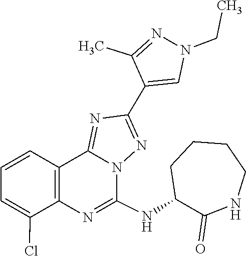 [SMILES: CCn1cc(-c2nc3c4cccc(Cl)c4nc(N[C@@H]4CCCCNC4=O)n3n2)c(C)n1] |
| | (3R)-3-{[7-chloro-2-(1-ethyl-3-methyl-1H-pyrazol-4-yl)[1,2,4]triazolo[1,5- |
| | c]quinazolin-5-yl]amino}azepan-2-one |
| | LC-MS (Method 2): Rₜ = 1.33 min; MS (ESIpos): m/z = 439 [M + H]⁺ |
| | ¹H-NMR (400 MHz, DMSO-d₆): δ [ppm] = 1.26-1.38 (m, 1H), 1.41 (t, 3H), |
| | 1.48-1.60 (m, 1H), 1.81-1.96 (m, 2H), 2.00-2.10 (m, 1H), 2.38-2.46 (m, |
| | 1H), 2.58 (d, 3H), 3.11-3.24 (m, 1H), 3.27-3.32 (m, 1H), 4.16 (q, 2H), 4.81 |
| | (dd, 1H), 7.39 (t, 1H), 7.74 (d, 1H), 7.88 (dd, 1H), 8.18 (dd, 1H), 8.22 (dd, |
| | 1H), 8.41 (s, 1H). |
| Example 820 | 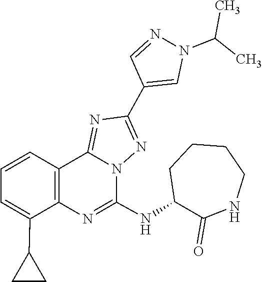 [SMILES: CC(C)n1cc(-c2nc3c4cccc(C5CC5)c4nc(N[C@@H]4CCCCNC4=O)n3n2)cn1] |
| | (3R)-3-({7-cyclopropyl-2-[1-(propan-2-yl)-1H-pyrazol-4- |
| | yl][1,2,4]triazolo[1,5-c]quinazolin-5-yl}amino)azepan-2-one |
| | LC-MS (Method 2): Rt = 1.32 min; MS (ESIpos): m/z = 445 [M + H]⁺ |
| | ¹H-NMR (400 MHz, DMSO-d₆): δ [ppm] = 0.77-0.86 (m, 1H), 0.87-0.95 (m, |
| | 1H), 1.01-1.13 (m, 2H), 1.20-1.27 (m, 1H), 1.28-1.39 (m, 1H), 1.49 (d, |
| | 6H), 1.53-1.60 (m, 1H), 1.77-1.93 (m, 2H), 1.99-2.07 (m, 1H), 2.42 (br d, |
| | 1H), 2.91 (tt, 1H), 3.11-3.21 (m, 1H), 4.63 (spt, 1H), 4.85 (dd, 1H), 7.24- |
| | 7.28 (m, 1H), 7.30-7.36 (m, 1H), 7.54 (d, 1H), 8.04 (dd, 1H), 8.08 (s, 1H), |
| | 8.22 (dd, 1H), 8.53 (s, 1H). |
| Example 821 | 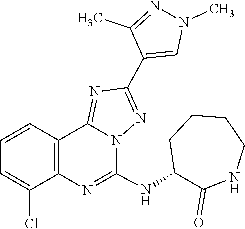 [SMILES: Cc1nn(C)cc1-c1nc2c3cccc(Cl)c3nc(N[C@@H]3CCCCNC3=O)n2n1] |
| | (3R)-3-{[7-chloro-2-(1,3-dimethyl-1H-pyrazol-4-yl)[1,2,4]triazolo[1,5- |
| | c]quinazolin-5-yl]amino}azepan-2-one |
| | LC-MS (Method 2): Rₜ = 1.23 min; MS (ESIneg): m/z = 423 [M − H]⁻ |
| | ¹H-NMR (400 MHz, DMSO-d₆): δ [ppm] = 1.26-1.41 (m, 1H), 1.47-1.60 (m, |
| | 1H), 1.81-1.96 (m, 2H), 2.00-2.08 (m, 1H), 2.39-2.46 (m, 1H), 2.58 (s, |
| | 3H), 3.12-3.25 (m, 1H), 3.86 (s, 3H), 4.81 (br dd, 1H), 7.39 (t, 1H), 7.74 (d, |
| | 1H), 7.89 (dd, 1H), 8.16-8.25 (m, 2H), 8.38 (s, 1H). |
| Example 822 | 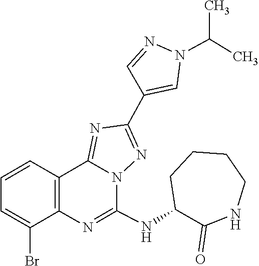 [SMILES: CC(C)n1cc(-c2nc3c4cccc(Br)c4nc(N[C@@H]4CCCCNC4=O)n3n2)cn1] |
| | (3R)-3-({7-bromo-2-[1-(propan-2-yl)-1H-pyrazol-4-yl][1,2,4]triazolo[1,5- |
| | c]quinazolin-5-yl}amino)azepan-2-one |
| | LC-MS (Method 2): Rₜ = 1.34 min; MS (ESIpos): m/z = 483 [M + H]⁺ |
| | ¹H-NMR (400 MHz, DMSO-d₆): δ [ppm] = 1.17-1.42 (m, 2H), 1.50 (d, 6H), |
| | 1.81-1.99 (m, 2H), 2.00-2.11 (m, 1H), 2.39-2.47 (m, 1H), 3.13-3.25 (m, |
| | 1H), 3.26-3.31 (m, 1H), 4.64 (dt, 1H), 4.84 (br dd, 1H), 7.34 (t, 1H), 7.71 (d, |
| | 1H), 8.04-8.12 (m, 2H), 8.25 (dd, 2H), 8.55 (s, 1H). |
| Example 823 | 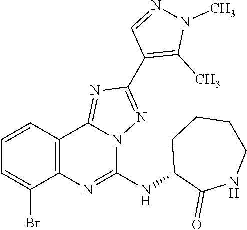 [SMILES: Cc1c(-c2nc3c4cccc(Br)c4nc(N[C@@H]4CCCCNC4=O)n3n2)cnn1C] |
| | (3R)-3-{[7-bromo-2-(1,5-dimethyl-1H-pyrazol-4-yl)[1,2,4]triazolo[1,5- |
| | c]quinazolin-5-yl]amino}azepan-2-one |
| | LC-MS (Method 2): Rₜ = 1.28 min; MS (ESIpos): m/z = 471 [M + H]⁺ |
| | ¹H-NMR (400 MHz, DMSO-d₆): δ [ppm] = 1.49-1.62 (m, 1H), 1.82-2.00 (m, |
| | 2H), 2.01-2.13 (m, 1H), 2.39-2.47 (m, 1H), 2.76 (s, 3H), 3.13-3.24 (m, |
| | 1H), 3.85 (s, 3H), 4.83 (dd, 1H), 7.33 (t, 1H), 7.76 (d, 1H), 7.99 (s, 1H), 8.06 |
| | (dd, 1H), 8.22 (br dd, 1H), 8.27 (dd, 1H). |
| Example 824 | 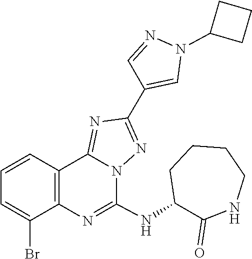 [SMILES: O=C1NCCCC[C@H]1Nc1nc2c(Br)cccc2c2nc(-c3cnn(C4CCC4)c3)nn12] |
| | (3R)-3-{[7-bromo-2-(1-cyclobutyl-1H-pyrazol-4-yl)[1,2,4]triazolo[1,5- |
| | c]quinazolin-5-yl]amino}azepan-2-one |
| | LC-MS (Method 2): Rₜ = 1.38 min; MS (ESIpos): m/z = 497 [M + H]⁺ |
| | ¹H-NMR (400 MHz, DMSO-d₆): δ [ppm] = 1.27-1.41 (m, 1H), 1.46-1.60 (m, |
| | 1H), 1.74-1.89 (m, 3H), 1.90-2.00 (m, 1H), 2.02-2.11 (m, 1H), 2.37-2.46 |
| | (m, 3H), 2.55-2.65 (m, 2H), 3.15-3.25 (m, 1H), 3.25-3.30 (m, 1H), 4.83 |
| | (br dd, 1H), 4.97 (quin, 1H), 7.34 (t, 1H), 7.71 (d, 1H), 8.07 (dd, 1H), 8.13 (s, |
| | 1H), 8.25 (dd, 2H), 8.61 (s, 1H). |
| Example 825 | 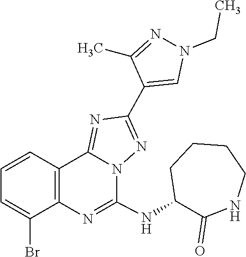 [SMILES: CCn1cc(-c2nc3c4cccc(Br)c4nc(N[C@@H]4CCCCNC4=O)n3n2)c(C)n1] |
| | (3R)-3-{[7-bromo-2-(1-ethyl-3-methyl-1H-pyrazol-4-yl)[1,2,4]triazolo[1,5- |
| | c]quinazolin-5-yl]amino}azepan-2-one |
| | LC-MS (Method 2): Rₜ = 1.34 min; MS (ESIpos): m/z = 485 [M + H]⁺ |
| | ¹H-NMR (400 MHz, DMSO-d₆): δ [ppm] = 1.27-1.38 (m, 1H), 1.42 (t, 3H), |
| | 1.48-1.61 (m, 1H), 1.82-2.00 (m, 2H), 2.01-2.12 (m, 1H), 2.45 (br d, 1H), |
| | 2.59 (s, 3H), 3.14-3.24 (m, 1H), 3.29 (m, 1H overlay with water signal), |
| | 4.16 (q, 2H), 4.82 (dd, 1H), 7.33 (t, 1H), 7.74 (d, 1H), 8.06 (dd, 1H), 8.20- |
| | 8.27 (m, 2H), 8.42 (s, 1H). |
| Example 826 | 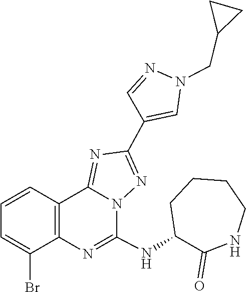 [SMILES: O=C1NCCCC[C@H]1Nc1nc2c(Br)cccc2c2nc(-c3cnn(CC4CC4)c3)nn12] |
| | (3R)-3-({7-bromo-2-[1-(cyclopropylmethyl)-1H-pyrazol-4- |
| | yl][1,2,4]triazolo[1,5-c]quinazolin-5-yl}amino)azepan-2-one |
| | LC-MS (Method 2): Rₜ = 1.33 min; MS (ESIpos): m/z = 497 [M + H]⁺ |
| | ¹H-NMR (400 MHz, DMSO-d₆): δ [ppm] = 0.40-0.48 (m, 2H), 0.53-0.61 (m, |
| | 2H), 1.26-1.42 (m, 2H), 1.47-1.60 (m, 1H), 1.82-2.01 (m, 2H), 2.04 (br s, |
| | 1H), 2.43 (br d, 1H), 3.14-3.25 (m, 1H), 3.26-3.31 (m, 1H), 4.08 (d, 2H), |
| | 4.83 (dd, 1H), 7.34 (t, 1H), 7.71 (d, 1H), 8.07 (dd, 1H), 8.10 (d, 1H), 8.26 |
| | (dd, 2H), 8.56 (s, 1H). |
| Example 827 | 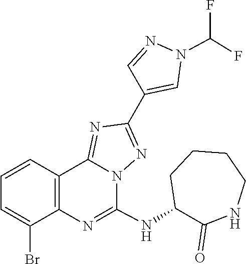 [SMILES: O=C1NCCCC[C@H]1Nc1nc2c(Br)cccc2c2nc(-c3cnn(C(F)F)c3)nn12] |
| | (3R)-3-({7-bromo-2-[1-(difluoromethyl)-1H-pyrazol-4- |
| | yl][1,2,4]triazolo[1,5-c]quinazolin-5-yl}amino)azepan-2-one |
| | LC-MS (Method 2): Rₜ = 1.33 min; MS (ESIneg): m/z = 489 [M − H]⁻ |
| | ¹H-NMR (400 MHz, DMSO-d₆): δ [ppm] = 1.28-1.43 (m, 1H), 1.48-1.60 (m, |
| | 1H), 1.83-2.02 (m, 2H), 2.02-2.14 (m, 1H), 2.40-2.47 (m, 1H), 3.15-3.25 |
| | (m, 1H), 3.27-3.34 (m, 1H, and water signal), 4.84 (dd, 1H), 7.36 (t, 1H), |
| | 7.74-7.77 (m, 1H), 7.77-8.08 (m, 1H), 8.09 (dd, 1H), 8.27 (dd, 2H), 8.45 |
| | (s, 1H), 9.04 (s, 1H). |
| Example 828 | 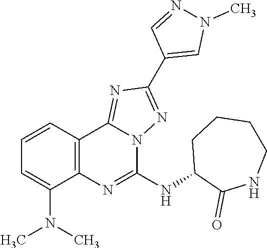 [SMILES: CN(C)c1cccc2c1nc(N[C@@H]1CCCCNC1=O)n1nc(-c3cnn(C)c3)nc21] |
| | (3R)-3-{[7-(dimethyalmino)-2-(1-methyl-1H-pyrazol-4- |
| | yl)[1,2,4]triazolo[1,5-c]quinazolin-5-yl]amino}azepan-2-one |
| | LC-MS (Method 2): Rₜ = 1.05 min; MS (ESIpos): m/z = 419 [M]⁺ |
| | ¹H-NMR (400 MHz, DMSO-d₆): δ [ppm] = 1.26-1.41 (m, 2H), 1.51-1.62 (m, |
| | 1H), 1.79-1.91 (m, 2H), 1.96-2.06 (m, 1H), 2.33-2.39 (m, 1H), 3.04 (s, |
| | 6H), 3.11-3.21 (m, 1H), 3.95 (s, 3H), 4.82 (br dd, 1H), 7.15 (dd, 1H), 7.31 |
| | (t, 1H), 7.56 (d, 1H), 7.78 (dd, 1H), 8.06 (s, 1H), 8.22 (br dd, 1H), 8.48 (s, |
| | 1H). |
| Example 829 | 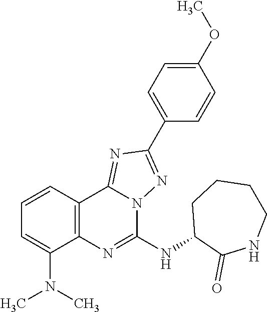 [SMILES: COc1ccc(-c2nc3c4cccc(N(C)C)c4nc(N[C@@H]4CCCCNC4=O)n3n2)cc1] |
| | (3R)-3-{[7-(dimethylamino)-2-(4-methoxyphenyl)[1,2,4]triazolo[1,5- |
| | c]quinazolin-5-yl]amino}azepan-2-one |
| | LC-MS (Method 2): Rₜ = 1.34 min; MS (ESIpos): m/z = 446 [M + H]⁺ |
| | ¹H-NMR (400 MHz, DMSO-d₆): δ [ppm] = 1.33 (br d, 1H), 1.54-1.65 (m, 1H), |
| | 1.81-1.91 (m, 2H), 2.00-2.06 (m, 1H), 2.34-2.40 (m, 1H), 3.04 (s, 6H), |
| | 3.14-3.22 (m, 1H), 3.86 (s, 3H), 4.83 (dd, 1H), 7.12-7.15 (m, 2H), 7.16- |
| | 7.19 (m, 1H), 7.29-7.35 (m, 1H), 7.67 (d, 1H), 7.83 (dd, 1H), 8.19-8.23 (m, |
| | 2H). |
| Example 830 | 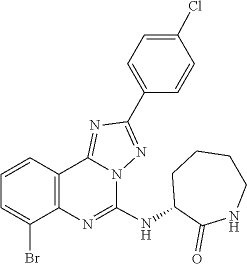 [SMILES: O=C1NCCCC[C@H]1Nc1nc2c(Br)cccc2c2nc(-c3ccc(Cl)cc3)nn12] |
| | (3R)-3-{[7-bromo-2-(4-chlorophenyl)[1,2,4]triazolo[1,5-c]quinazolin-5- |
| | yl]amino}azepan-2-one |
| | LC-MS (Method 2): Rₜ = 1.59 min; MS (ESIpos): m/z = 486 [M + H]⁺ |
| | ¹H-NMR (400 MHz, DMSO-d): δ [ppm] = 1.28-1.40 (m, 1H), 1.50-1.61 (m, |
| | 1H), 1.83-2.00 (m, 2H), 2.01-2.09 (m, 1H), 2.41-2.47 (m, 1H), 3.15-3.24 |
| | (m, 1H), 3.27-3.30 (m, 1H), 4.84 (br dd, 1H), 7.36 (t, 1H), 7.64-7.68 (m, |
| | 2H), 7.85 (d, 1H), 8.09 (dd, 1H), 8.24-8.27 (m, 1H), 8.27-8.30 (m, 2H), |
| | 8.30-8.33 (m, 1H). |
| Example 831 | 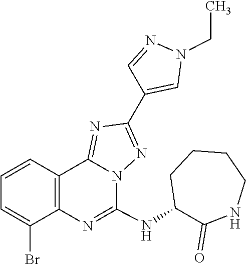 [SMILES: CCn1cc(-c2nc3c4cccc(Br)c4nc(N[C@@H]4CCCCNC4=O)n3n2)cn1] |
| | (3R)-3-{[7-bromo-2-(1-ethyl-1H-pyrazol-4-yl)[1,2,4]triazolo[1,5- |
| | c]quinazolin-5-yl]amino}azepan-2-one |
| | LC-MS (Method 2): Rₜ = 1.26 min; MS (ESIpos): m/z = 469 [M + H]⁺ |
| | ¹H-NMR (400 MHz, DMSO-d₆): δ [ppm] = 1.32 (br d, 1H), 1.50 (t, 3H), 1.81 |
| | (br dd, 1H), 1.88-2.02 (m, 2H), 2.34-2.40 (m, 1H), 3.08-3.26 (m, 2H), |
| | 4.27 (q, 2H), 4.67 (dd, 1H), 7.24 (d, 1H), 7.30 (t, 1H), 7.88 (s, 1H), 7.98 (dd, |
| | 1H), 8.06-8.13 (m, 1H), 8.29-8.33 (m, 2H). |
| Example 832 | 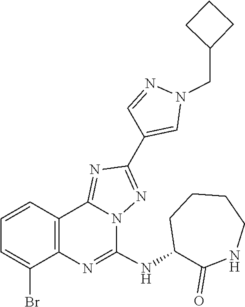 [SMILES: O=C1NCCCC[C@H]1Nc1nc2c(Br)cccc2c2nc(-c3cnn(CC4CCC4)c3)nn12] |
| | (3R)-3-({7-bromo-2-[1-(cyclobutylmethyl)-1H-pyrazol-4- |
| | yl][1,2,4]triazolo[1,5-c]quinazolin-5-yl}amino)azepan-2-one |
| | LC-MS (Method 2): Rₜ = 1.42 min; MS (ESIpos): m/z = 510 [M + H]⁺ |
| | ¹H-NMR (400 MHz, DMSO-d₆): δ [ppm] = 1.28-1.43 (m, 1H), 1.47-1.62 (m, |
| | 1H), 1.75-1.94 (m, 6H), 1.94-2.12 (m, 3H), 2.40-2.47 (m, 1H), 2.77-2.89 |
| | (m, 1H), 3.14-3.26 (m, 1H), 3.27-3.31 (m, 1H), 4.25 (d, 2H), 4.83 (dd, 1H), |
| | 7.34 (t, 1H), 7.70 (d, 1H), 8.06-8.10 (m, 2H), 8.22-8.29 (m, 2H), 8.51- |
| | 8.53 (m, 1H). |
| Example 833 | 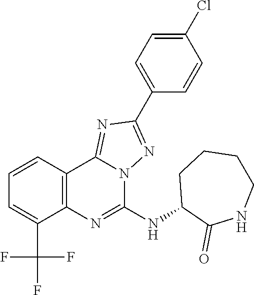 [SMILES: O=C1NCCCC[C@H]1Nc1nc2c(C(F)(F)F)cccc2c2nc(-c3ccc(Cl)cc3)nn12] |
| | (3R)-3-{[2-(4-chlorophenyl)-7-(trifluoromethyl)[1,2,4]triazolo[1,5- |
| | c]quinazolin-5-yl]amino}azepan-2-one |
| | LC-MS (Method 2): Rₜ = 1.60 min; MS (ESIpos): m/z = 475 [M + H]⁺ |
| | ¹H-NMR (400 MHz, DMSO-d₆): δ [ppm] = 1.27-1.37 (m, 1H), 1.49-1.59 (m, |
| | 1H), 1.82-1.91 (m, 2H), 2.03 (br d, 1H), 2.37-2.44 (m, 1H), 3.19-3.30 (m, |
| | 2H), 4.75 (br dd, 1H), 7.56 (t, 1H), 7.64-7.68 (m, 2H), 7.91 (d, 1H), 8.11 (d, |
| | 1H), 8.27 (s, 1H), 8.29 (s, 2H), 8.52-8.56 (m, 1H). |
| Example 834 | 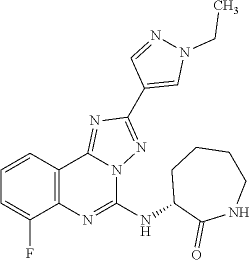 [SMILES: CCn1cc(-c2nc3c4cccc(F)c4nc(N[C@@H]4CCCCNC4=O)n3n2)cn1] |
| | (3R)-3-{[2-(1-ethyl-1H-pyrazol-4-yl)-7-fluoro[1,2,4]triazolo[1,5- |
| | c]quinazolin-5-yl]amino}azepan-2-one |
| | LC-MS (Method 2): Rₜ = 1.13 min; MS (ESIneg): m/z = 407 [M − H]⁻ |
| | ¹H-NMR (400 MHz, DMSO-d₆): δ [ppm] = 1.25-1.38 (m, 1H), 1.45 (t, 3H), |
| | 1.48-1.60 (m, 1H), 1.78-1.91 (m, 2H), 1.97-2.08 (m, 1H), 2.33-2.39 (m, |
| | 1H), 3.10-3.20 (m, 1H), 3.35-3.40 (m, 1H), 4.25 (q, 2H), 4.82 (dd, 1H), |
| | 7.40 (td, 1H), 7.57-7.62 (m, 1H), 7.73 (d, 1H), 8.03-8.06 (m, 1H), 8.09 (s, |
| | 1H), 8.23 (dd, 1H), 8.54 (d, 1H). |
| Example 835 | 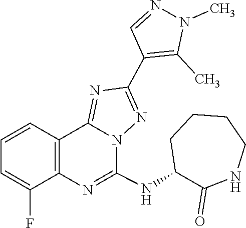 [SMILES: Cc1c(-c2nc3c4cccc(F)c4nc(N[C@@H]4CCCCNC4=O)n3n2)cnn1C] |
| | (3R)-3-{[2-(1,5-dimethyl-1H-pyrazol-4-yl)-7-fluoro[1,2,4]triazolo[1,5- |
| | c]quinaozlin-5-yl]amino}azepan-2-one |
| | LC-MS (Method 2): Rₜ = 1.14 min; MS (ESIneg): m/z = 407 [M − H]⁻ |
| | ¹H-NMR (400 MHz, DMSO-d₆): δ [ppm] = 1.25-1.38 (m, 1H), 1.50-1.62 (m, |
| | 1H), 1.81-1.90 (m, 2H), 1.98-2.06 (m, 1H), 2.29-2.38m, 1H), 2.75 (s, 3H), |
| | 3.12-3.20 (m, 1H), 3.29-3.39 (m, 1H and water signal), 3.84 (s, 3H), 4.82 |
| | (dd, 1H), 7.39 (td, 1H), 7.59 (ddd, 1H), 7.79 (d, 1H), 7.99 (s, 1H), 8.05-8.09 |
| | (m, 1H), 8.20 (dd, 1H). |
| Example 836 | 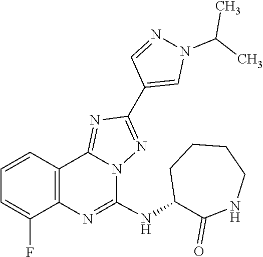 [SMILES: CC(C)n1cc(-c2nc3c4cccc(F)c4nc(N[C@@H]4CCCCNC4=O)n3n2)cn1] |
| | (3R)-3-({7-fluoro-2-[1-(propan-2-yl)-1H-pyrazol-4-yl][1,2,4]triazolo∥,5- |
| | c]quinazolin-5-yl}amino)azepan-2-one |
| | LC-MS (Method 2): Rₜ = 1.20 min; MS (ESIneg): m/z = 421 [M − H]⁻ |
| | ¹H-NMR (400 MHz, DMSO-d₆): δ [ppm] = 1.28-1.42 (m, 1H), 1.49 (d, 6H), |
| | 1.51-1.61 (m, 1H), 1.80-1.91 (m, 2H), 1.98-2.06 (m, 1H), 2.34 (br d, 1H), |
| | 3.12-3.21 (m, 1H), 3.30-3.32 (m, 1H), 4.64 (quin, 1H), 4.82 (dd, 1H), 7.40 |
| | (td, 1H), 7.59 (ddd, 1H), 7.74 (d, 1H), 8.02-8.07 (m, 1H), 8.09 (s, 1H), 8.23 |
| | (dd, 1H), 8.54 (s, 1H). |
| Example 837 | 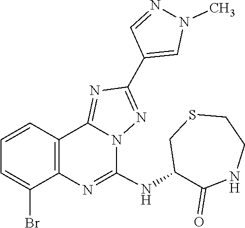 [SMILES: Cn1cc(-c2nc3c4cccc(Br)c4nc(N[C@@H]4CSCCNC4=O)n3n2)cn1] |
| | (6S)-6-{[7-bromo-2-(1-methyl-1H-pyrazol-4-yl)[1,2,4]triazolo[1,5- |
| | c]quinazolin-5-yl]amino}-1,4-thiazepan-5-one |
| | LC-MS (Method 2): Rt = 1.14 min; MS (ESIneg): m/z = 471 [M − H]⁻ |
| Example 838 | 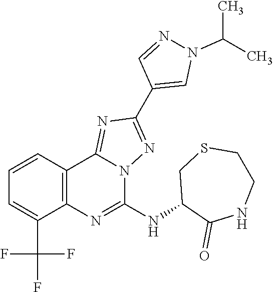 [SMILES: CC(C)n1cc(-c2nc3c4cccc(C(F)(F)F)c4nc(N[C@@H]4CSCCNC4=O)n3n2)cn1] |
| | (6S)-6-({2-[1-(propan-2-yl)-1H-pyrazol-4-yl]-7- |
| | (trifluoromethyl)[1,2,4]triaozlo[1,5-c]quinazolin-5-yl}amino)-1,4- |
| | thiazepan-5-one |
| | LC-MS (Method 2): Rₜ = 1.29 min; MS (ESIpos): m/z = 491 [M + H]⁺ |
| | ¹H-NMR (500 MHz, DMSO-d₆): δ [ppm] = 1.50 (d, 6H), 2.56-2.68 (m, 2H), |
| | 2.71-2.80 (m, 1H), 2.80-2.89 (m, 1H), 3.16 (d, 1H), 3.56-3.66 (m, 2H), |
| | 4.64 (spt, 1H), 7.56 (t, 1H), 7.96 (d, 1H), 8.08-8.11 (m, 2H), 8.41 (t, 1H), |
| | 8.51 (dd, 1H), 8.54 (s, 1H). |
| Example 839 | 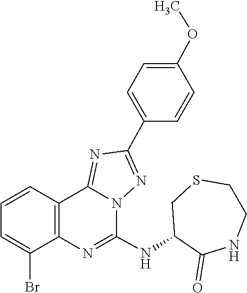 [SMILES: COc1ccc(-c2nc3c4cccc(Br)c4nc(N[C@@H]4CSCCNC4=O)n3n2)cc1] |
| | (6S)-6-{[7-bromo-2-(4-methoxyphenyl)[1,2,4]triazolo[1,5-c]quinazolin-5- |
| | yl]amino}-1,4-thiazepan-5-one |
| | LC-MS (Method 2): Rₜ = 1.42 min; MS (ESIpos): m/z = 499 [M + H]⁺ |
| | ¹H-NMR (500 MHz, DMSO-d₆): δ [ppm] = 2.61-2.69 (m, 1H), 2.73-2.79 (m, |
| | 1H), 2.85-2.91 (m, 1H), 3.22-3.31 (m, 1H), 3.58-3.72 (m, 2H), 3.86 (s, |
| | 3H), 5.20-5.27 (m, 1H), 7.13-7.17 (m, 2H), 7.36 (t, 1H), 7.96 (d, 1H), 8.08 |
| | (dd, 1H), 8.20-8.24 (m, 2H), 8.31 (dd, 1H), 8.40 (t, 1H). |
| Example 840 | 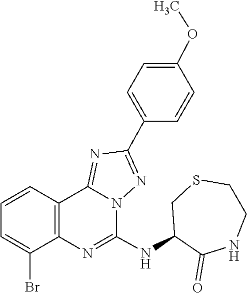 [SMILES: COc1ccc(-c2nc3c4cccc(Br)c4nc(N[C@H]4CSCCNC4=O)n3n2)cc1] |
| | (6R)-6-{[7-bromo-2-(4-methoxyphenyl)[1,2,4]triazolo[1,5-c]quinazolin-5- |
| | yl]amino}-1,4-thiazepan-5-one |
| | LC-MS (Method 2): Rₜ = 1.43 min; MS (ESIpos): m/z = 500 [M + H]⁺ |
| | ¹H-NMR (400 MHz, DMSO-d₆): δ [ppm] = 2.67 (br d, 1H), 2.72-2.80 (m, 1H), |
| | 2.88 (dd, 1H), 3.24 (br d, 1H), 3.64 (br s, 2H), 3.86 (s, 3H), 5.21-5.28 (m, |
| | 1H), 7.13-7.17 (m, 1H), 7.15 (d, 1H), 7.36 (t, 1H), 7.96 (d, 1H), 8.08 (d, 1H), |
| | 8.22 (d, 2H), 8.31 (d, 1H), 8.38-8.43 (m, 1H). |
| Example 841 | 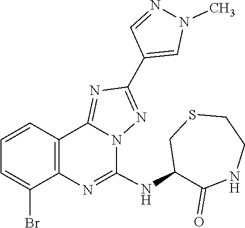 [SMILES: Cn1cc(-c2nc3c4cccc(Br)c4nc(N[C@H]4CSCCNC4=O)n3n2)cn1] |
| | (6R)-6-{[7-bromo-2-(1-methyl-1H-pyrazol-4-yl)[1,2,4]triazolo[1,5- |
| | c]quinazolin-5-yl]amino}-1,4-thiazepan-5-one |
| | LC-MS (Method 2): Rₜ = 1.11 min; MS (ESIpos): m/z = 473 [M + H]⁺ |
| | ¹H-NMR (400 MHz, DMSO-d₆): δ [ppm] = 2.60-2.69 (m, 1H), 2.71-2.79 (m, |
| | 1H), 2.86 (dd, 1H), 3.23 (d, 1H), 3.54-3.72 (m, 2H), 3.92-3.99 (m, 3H), |
| | 5.23 (br dd, 1H), 7.35 (t, 1H), 7.85 (d, 1H), 8.03-8.12 (m, 2H), 8.25 (dd, |
| | 1H), 8.37-8.49 (m, 1H), 8.50 (s, 1H). |

### Example 842

### (3R)-3-{[7-(methanesulfonyl)-2-(4-methoxyphenyl)[1,2,4]triazolo[1,5-c]quinazolin-5-yl]amino}azepan-2-one

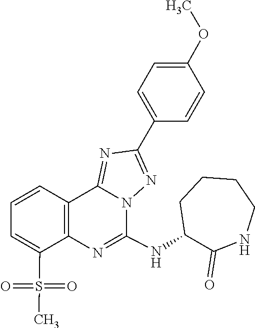 [SMILES: COc1ccc(-c2nc3c4cccc(S(C)(=O)=O)c4nc(N[C@@H]4CCCCNC4=O)n3n2)cc1]

(3R)-3-{[2-(4-Methoxyphenyl)-7-(methylsulfanyl)[1,2,4]triazolo[1,5-c]quinazolin-5-yl]amino}azepan-2-one (75.0 mg, 167 μmol) and mCPBA (93.7 mg, 77% purity, 418 μmol) were stirred in dichloromethane (6.9 mL) for 2 h at rt. The mixture was diluted with aqueous sodium thiosulfate solution (10%) and extracted with dichloromethane. The combined organic layers were dried over a silicone filter and concentrated under reduced pressure. The crude material was purified by preparative HPLC to give 58.2 mg (95% purity, 69% yield) of the title compound.

LC-MS (method 2): Rₜ=1.13 min; MS (ESIpos): m/z=482 [M+H]⁺

¹H-NMR (400 MHz, DMSO-d6): δ [ppm]=1.26-1.40 (m, 1H), 1.55-1.67 (m, 1H), 1.83-1.98 (m, 2H), 1.98-2.08 (m, 1H), 2.40-2.47 (m, 1H), 3.15-3.31 (m, 2H), 3.57 (s, 3H), 3.86 (s, 3H), 4.86 (br dd, 1H), 7.15 (d, 2H), 7.61 (t, 1H), 8.13 (d, 1H), 8.20-8.33 (m, 4H), 8.61 (dd, 1H).

The following examples were prepared similarly to example 842

| | Structure |
|---|---|
| | IUPAC-Name |
| | LC-MS (method): Retention time; Mass found |
| Example | ¹H-NMR |
| Example 843 | 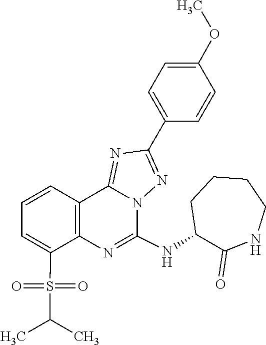 [SMILES: COc1ccc(-c2nc3c4cccc(S(=O)(=O)C(C)C)c4nc(N[C@@H]4CCCCNC4=O)n3n2)cc1] |
| | (3R)-3-{[2-(4-methoxyphenyl)-7-(propane-2-sulfonyl)[1,2,4]triazolo[1,5- |
| | c]quinazolin-5-yl]amino}azepan-2-one |
| | LC-MS (Method 2): Rₜ = 1.20 min; MS (ESIpos): m/z = 509 [M + H]⁺ |
| | ¹H-NMR (400 MHz, DMSO-d₆): δ [ppm] = 1.10 (d, 3H), 1.25-1.40 (m, 4H), |
| | 1.58-1.72 (m, 1H), 1.82-1.94 (m, 2H), 1.98-2.09 (m, 1H), 2.31-2.40 (m, |
| | 1H), 3.14-3.30 (m, 2H), 3.87 (s, 3H), 4.39 (spt, 1H), 4.82 (br dd, 1H), 7.12- |
| | 7.19 (m, 2H), 7.62 (t, 1H), 8.15-8.32 (m, 5H), 8.62 (dd, 1H). |
| Example 844 | 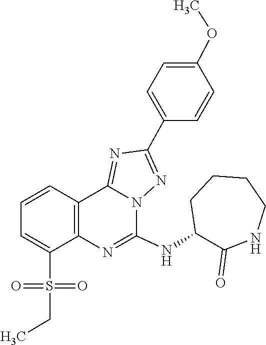 [SMILES: CCS(=O)(=O)c1cccc2c1nc(N[C@@H]1CCCCNC1=O)n1nc(-c3ccc(OC)cc3)nc21] |
| | (3R)-3-{[7-(ethanesulfonyl)-2-(4-methoxyphenyl)[1,2,4]triazolo[1,5- |
| | c]quinazolin-5-yl]amino}azepan-2-one |
| | LC-MS (Method 2): Rₜ = 1.15 min; MS (ESIpos): m/z = 495 [M + H]⁺ |
| | ¹H-NMR (400 MHz, DMSO-d₆): δ [ppm] = 1.15 (t, 3H), 1.27-1.39 (m, 1H), |
| | 1.53-1.70 (m, 1H), 1.81-1.95 (m, 2H), 1.96-2.07 (m, 1H), 2.40 (br d, 1H), |
| | 3.14-3.31 (m, 2H), 3.67-3.79 (m, 1H), 3.80-3.91 (m, 4H), 4.84 (br dd, |
| | 1H), 7.13-7.20 (m, 2H), 7.62 (t, 1H), 8.15 (d, 1H), 8.20-8.33 (m, 4H), 8.62 |
| | (dd, 1H). |
| Example 845 | 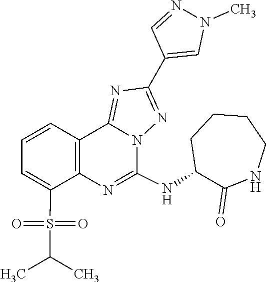 [SMILES: CC(C)S(=O)(=O)c1cccc2c1nc(N[C@@H]1CCCCNC1=O)n1nc(-c3cnn(C)c3)nc21] |
| | (3R)-3-{[2-(1-methyl-1H-pyrazol-4-yl)-7-(propane-2- |
| | sulfonyl)[1,2,4]triazolo[1,5-c]quinazolin-5-yl]amino}azepan-2-one |
| | LC-MS (Method 2): Rₜ = 0.93 min; MS (ESIpos): m/z = 483 [M + H]⁺ |
| | ¹H-NMR (400 MHz, DMSO-d₆): δ [ppm] = 1.10 (d, 3H), 1.26-1.37 (m, 4H), |
| | 1.54-1.69 (m, 1H), 1.80-1.93 (m, 2H), 1.98-2.07 (m, 1H), 2.30-2.42 (m, |
| | 1H), 3.14-3.30 (m, 2H), 3.96 (s, 3H), 4.32-4.45 (m, 1H), 4.81 (br dd, 1H), |
| | 7.60 (t, 1H), 8.06 (d, 1H), 8.09 (d, 1H), 8.23-8.29 (m, 2H), 8.51 (s, 1H), 8.56 |
| | (dd, 1H). |
| Example 846 | 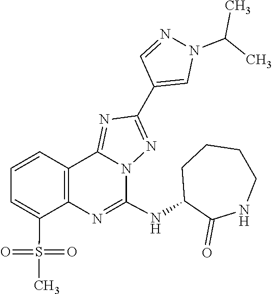 [SMILES: CC(C)n1cc(-c2nc3c4cccc(S(C)(=O)=O)c4nc(N[C@@H]4CCCCNC4=O)n3n2)cn1] |
| | (3R)-3-({7-(methanesulfonyl)-2-[1-(propan-2-yl)-1H-pyrazol-4- |
| | yl][1,2,4]triazolo[1,5-c]quinazolin-5-yl}amino)azepan-2-one |
| | LC-MS (Method 2): Rₜ = 1.01 min; MS (ESIpos): m/z = 483 [M + H]⁺ |
| | ¹H-NMR (400 MHz, DMSO-d₆): δ [ppm] = 1.33 (br d, 2H), 1.5 (d, 6H), 1.58 |
| | (br d, 1H), 1.83-1.96 (m, 2H), 1.97-2.07 (m, 1H), 2.44 (br d, 1H), 3.14- |
| | 3.26 (m, 1H), 3.57 (s, 3H), 4.65 (spt, 1H), 4.86 (br d, 1H), 7.61 (t, 1H), 8.02 |
| | (br d, 1H), 8.08-8.13 (m, 1H), 8.30 (br dd, 2H), 8.52-8.60 (m, 2H). |
| Example 847 | 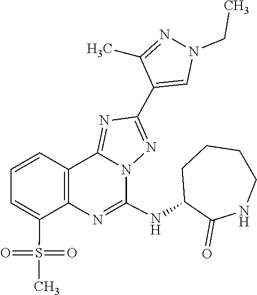 [SMILES: CCn1cc(-c2nc3c4cccc(S(C)(=O)=O)c4nc(N[C@@H]4CCCCNC4=O)n3n2)c(C)n1] |
| | (3R)-3-{[2-(1-ethyl-3-methyl-1H-pyrazol-4-yl)-7- |
| | (methanesulfonyl)[1,2,4]triazolo[1,5-c]quinazolin-5-yl]amino}azepan-2- |
| | one |
| | LC-MS (Method 2): Rₜ = 1.00 min; MS (ESIpos): m/z = 483 [M + H]⁺ |
| | ¹H-NMR (400 MHz, DMSO-d₆): δ [ppm] = 1.27-1.38 (m, 1H), 1.42 (t, 3H), |
| | 1.59 (q, 1H), 1.83-1.97 (m, 2H), 1.98-2.05 (m, 1H), 2.44 (br s, 1H), 2.60 |
| | (s, 3H), 3.15-3.31 (m, 2H), 3.57 (s, 3H), 4.17 (q, 2H), 4.84 (br dd, 1H), 7.60 |
| | (t, 1H), 8.04 (d, 1H), 8.23-8.33 (m, 2H), 8.43 (s, 1H), 8.55 (dd, 1H). |

### Example 848

### (6R)-6-{[7-bromo-2-(1-methyl-1H-pyrazol-4-yl)[1,2,4]triazolo[1,5-c]quinazolin-5-yl]amino}-1λ⁶,4-thiazepane-1,1,5-trione

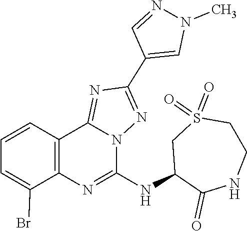 [SMILES: Cn1cc(-c2nc3c4cccc(Br)c4nc(N[C@H]4CS(=O)(=O)CCNC4=O)n3n2)cn1]

(6R)-6-{[7-Bromo-2-(1-methyl-1H-pyrazol-4-yl)[1,2,4]triazolo[1,5-c]quinazolin-5-yl]amino}-1,4-thiazepan-5-one (50.0 mg, 106 μmol), mCPBA (47.3 mg, 77% purity, 211 μmol) and sodium hydrogen carbonate (17.7 mg, 211 μmol) were stirred in dichloromethane (3.0 mL) for 18 h at rt. The mixture was then diluted with aqueous sodium thiosulfate solution (10%). Aqueous Sodium carbonate solution (2N) was added and the suspension was extracted with ethyl acetate. The combined organic phases were dried (silicon filter) and concentrated under reduced pressure. The crude mixture was purified by preparative HPLC to give 4.90 mg (97% purity, 9% yield) of the title compound.

LC-MS (method 2): Rₜ=0.92 min; MS (ESIpos): m/z=505 [M+H]⁺

¹H-NMR (400 MHz, DMSO-d6): δ [ppm]=3.36-3.47 (m, 2H), 3.50-3.62 (m, 1H), 3.66-3.85 (m, 3H), 3.96 (s, 3H), 5.34 (br d, 1H), 7.36 (t, 1H), 8.07 (td, 2H), 8.22 (br s, 1H), 8.26 (dd, 1H), 8.48 (s, 1H), 8.55 (t, 1H).

### Example 849

### (6R)-6-{[7-bromo-2-(1-methyl-1H-pyrazol-4-yl)[1,2,4]triazolo[1,5-c]quinazolin-5-yl]amino}-1λ⁴,4-thiazepane-1,5-dione (diastereoisomer 1)

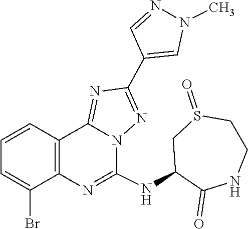 [SMILES: Cn1cc(-c2nc3c4cccc(Br)c4nc(N[C@H]4CS(=O)CCNC4=O)n3n2)cn1]

1.70 mg (90% purity, 3% yield) of the title compound were isolated from the previous reaction along with example 648

LC-MS (method 2): Rₜ=0.84 min; MS (ESIpos): m/z=489 [M+H]⁺

### Example 850

### (6R)-6-{[7-bromo-2-(1-methyl-1H-pyrazol-4-yl)[1,2,4]triazolo[1,5-c]quinazolin-5-yl]amino}-1λ⁴,4-thiazepane-1,5-dione (diastereoisomer 2)

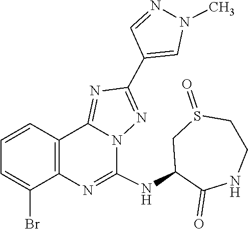 [SMILES: Cn1cc(-c2nc3c4cccc(Br)c4nc(N[C@H]4CS(=O)CCNC4=O)n3n2)cn1]

2.60 mg (96% purity, 5% yield) of the title compound were isolated from the previous reaction along with example 648

LC-MS (method 2): Rₜ=0.80 min; MS (ESIpos): m/z=489 [M+H]⁺

### Example 851

### (6S)-6-{[7-bromo-2-(4-methoxyphenyl)[1,2,4]triazolo[1,5-c]quinazolin-5-yl]amino}-1λ⁶,4-thiazepane-1,1,5-trione

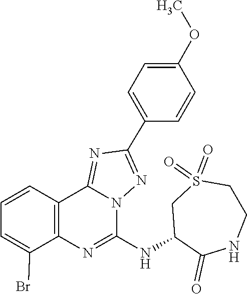 [SMILES: COc1ccc(-c2nc3c4cccc(Br)c4nc(N[C@@H]4CS(=O)(=O)CCNC4=O)n3n2)cc1]

(6S)-6-{[7-Bromo-2-(4-methoxyphenyl)[1,2,4]triazolo[1,5-c]quinazolin-5-yl]amino}-1,4-thiazepan-5-one (30.0 mg, 60.1 μmol) and mCPBA (104 mg, 601 μmol) where suspended in 1,2-dichloroethane and stirred 2 h at 80° C. The mixture was diluted with aqueous Sodiumthiosulfate solution (10%) and 2N aqueous Sodium carbonate solution was added. The suspension was extracted with dichloromethane. The combined organic phases were dried (silicon filter) and concentrated under reduced pressure. The crude mixture was purified by preparative HPLC to give 4.00 mg (100% purity, 13% yield) of the title compound.

LC-MS (method 2): Rₜ=1.22 min; MS (ESIpos): m/z=531 [M+H]⁺

¹H-NMR (400 MHz, DMSO-d6): δ [ppm]=3.36-3.48 (m, 2H), 3.51-3.63 (m, 1H), 3.71-3.85 (m, 3H), 3.86 (s, 3H), 5.35 (br d, 1H), 7.14-7.18 (m, 2H), 7.37 (t, 1H), 8.08 (dd, 1H), 8.21-8.26 (m, 2H), 8.26-8.38 (m, 2H), 8.50-8.60 (m, 1H).

### Example 852

### (6S)-6-({2-[1-(propan-2-yl)-1H-pyrazol-4-yl]-7-(trifluoromethyl)[1,2,4]triazolo[1,5-c]quinazolin-5-yl}amino)-1λ⁶,4-thiazepane-1,1,5-trione

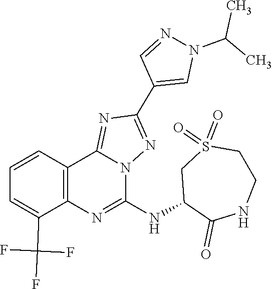 [SMILES: CC(C)n1cc(-c2nc3c4cccc(C(F)(F)F)c4nc(N[C@@H]4CS(=O)(=O)CCNC4=O)n3n2)cn1]

(6S)-6-({2-[1-(Propan-2-yl)-1H-pyrazol-4-yl]-7-(trifluoromethyl)[1,2,4]triazolo[1,5-c]quinazolin-5-yl}amino)-1,4-thiazepan-5-one (70.0 mg, 143 μmol) and mCPBA (246 mg, 1.43 mmol) were dissolved in 1,2-dichloroethane (4.0 mL) and stirred 2 h at 80° C. The mixture was diluted with 2N aqueous sodium hydrogen carbonate solution and washed with brine. The organic phase was dried over sodium sulfate, filtered and concentrated under reduced pressure. The crude mixture was purified by preparative HPLC to give 22.1 mg (100% purity, 30% yield) of the title compound.

LC-MS (method 2): Rₜ=1.10 min; MS (ESIpos): m/z=523 [M+H]⁺

¹H-NMR (400 MHz, DMSO-d6): δ [ppm]=1.50 (d, 6H), 3.35-3.45 (m, 2H), 3.51-3.64 (m, 1H), 3.64-3.78 (m, 3H), 4.65 (spt, 1H), 5.25-5.34 (m, 1H), 7.57 (t, 1H), 8.07-8.12 (m, 2H), 8.46-8.55 (m, 4H).

### Example 853

### (3R)-3-{[7-(S-methanesulfonimidoyl)-2-(4-methoxyphenyl)[1,2,4]triazolo[1,5-c]quinazolin-5-yl]amino}azepan-2-one, mixture of diastereomers

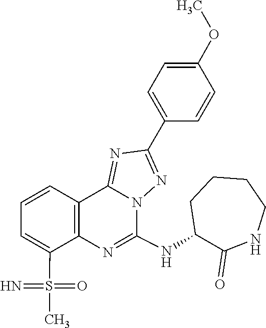 [SMILES: COc1ccc(-c2nc3c4cccc(S(C)(=N)=O)c4nc(N[C@@H]4CCCCNC4=O)n3n2)cc1]

(3R)-3-{[2-(4-Methoxyphenyl)-7-(methylsulfanyl)[1,2,4]triazolo[1,5-c]quinazolin-5-yl]amino}azepan-2-one (75.0 mg, 167 μmol), (diacetoxyiodo)benzene (135 mg, 418 μmol) and ammonium carbamate (26.1 mg, 334 μmol) were stirred in methanol (3.3 mL) overnight at rt. The mixture was concentrated and purified by preparative HPLC to give 46.7 mg (95% purity, 55% yield) of the title compound.

LC-MS (method 2): Rₜ=1.02 min; MS (ESIpos): m/z=480 [M+H]⁺

¹H-NMR (400 MHz, DMSO-d6): δ [ppm]=1.26-1.40 (m, 1H), 1.54-1.68 (m, 1H), 1.83-2.06 (m, 3H), 2.38-2.47 (m, 1H), 3.14-3.32 (m, 2H), 3.47 (s, 3H), 3.86 (s, 3H), 4.23-4.44 (m, 1H), 4.83-4.93 (m, 1H), 7.12-7.18 (m, 2H), 7.58 (td, 1H), 8.09 (dd, 1H), 8.20-8.29 (m, 3H), 8.33 (ddd, 1H), 8.53 (dd, 1H).

### Example 854

### (3R)-3-{[7-(methanesulfonimidoyl)-2-(4-methoxyphenyl)[1,2,4]triazolo[1,5-c]quinazolin-5-yl]amino}azepan-2-one, Diastereomer 1

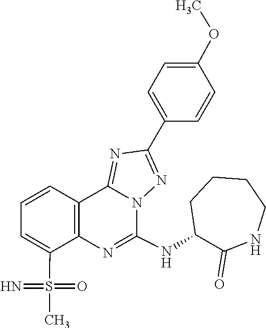 [SMILES: COc1ccc(-c2nc3c4cccc(S(C)(=N)=O)c4nc(N[C@@H]4CCCCNC4=O)n3n2)cc1]

Chiral HPLC separation of ((3R)-3-{[7-(S-methanesulfonimidoyl)-2-(4-methoxyphenyl)[1,2,4]triazolo[1,5-c]quinazolin-5-yl]amino}azepan-2-one (42.6 mg, 88.8 μmol) (example 853) was performed (Instrument: Labomatic HD5000, Labocord-5000; Gilson GX-241, Labcol Vario 4000, Column: Cellulose SC 5μ 250×30 mm; Eluent A: Acetonitrile+0.1 Vol-% Diethylamine (99%); Eluent B: Ethanol; Isocratic: 90% A+10% B; Flow rate 50.0 mL/min; UV 254 nm).

Retention time of diastereomer 1: 1.90 min; [α]²⁰_{D}: −18° (c=1) in DMSO.

Instrument: Agilent HPLC 1260; Column: Cellulose SC 3μ 100×4.6 mm; Eluent A: Acetonitrilel+0.1 Vol-% Diethylamine (99%); Eluent B: Ethanol; Isocratic: 90% A+10% B; Flow rate 1.4 mL/min; Temperature: 25° C.; DAD 254 nm

### Example 855

### (3R)-3-{[7-(methanesulfonimidoyl)-2-(4-methoxyphenyl)[1,2,4]triazolo[1,5-c]quinazolin-5-yl]amino}azepan-2-one, Diastereomer 2

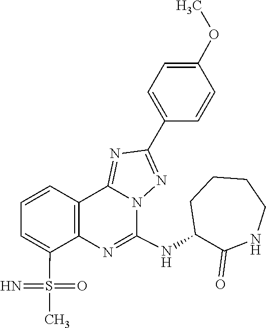 [SMILES: COc1ccc(-c2nc3c4cccc(S(C)(=N)=O)c4nc(N[C@@H]4CCCCNC4=O)n3n2)cc1]

The title compound was prepared as described for example 854.

Retention time of diastereomer 2: 2.40 min; [α]²⁰_{D}: −126° (c=1) in DMSO.

### Example 856

### (6R)-6-{[7-bromo-2-(1-methyl-1H-pyrazol-4-yl)[1,2,4]triazolo[1,5-c]quinazolin-5-yl]amino}-1-imino-1λ⁶,4-thiazepane-1,5-dione, Diastereomer 1

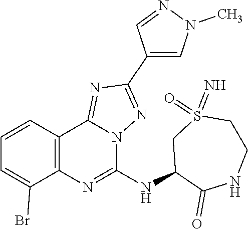 [SMILES: Cn1cc(-c2nc3c4cccc(Br)c4nc(N[C@H]4CS(=N)(=O)CCNC4=O)n3n2)cn1]

(6R)-6-{[7-Bromo-2-(1-methyl-1H-pyrazol-4-yl)[1,2,4]triazolo[1,5-c]quinazolin-5-yl]amino}-1,4-thiazepan-5-one (52.6 mg, 111 μmol), (diacetoxyiodo)benzene (89.5 mg, 278 μmol) and ammonium carbamate (17.4 mg, 222 μmol) were stirred in methanol (2.2 mL) overnight at rt. Ammonium carbamate (17.4 mg, 222 μmol) was again added and the reaction was stirred for 2 h at 40° C. (Diacetoxyiodo)benzene (89.5 mg, 278 μmol) and ammonium carbamate (17.4 mg, 222 μmol) were added and the reaction was stirred for another 48 h at 40° C. (Diacetoxyiodo)benzene (89.5 mg, 278 μmol) and ammonium carbamate (17.4 mg, 222 μmol) were again added and the reaction was stirred overnight at 40° C. The reaction mixture was concentrated under reduced pressure and purified by preparative HPLC to give 2.50 mg (93% purity, 4% yield) of the title compound

LC-MS (method 2): Rₜ=0.80 min; MS (ESIpos): m/z=504 [M]⁺

¹H-NMR (400 MHz, DMSO-d₆): δ [ppm]=3.13-3.26 (m, 2H), 3.52 (br dd, 2H), 3.60-3.69 (m, 1H), 3.72-3.84 (m, 1H), 3.96 (s, 3H), 4.20 (s, 1H), 5.30-5.48 (m, 1H), 7.35 (t, 1H), 8.01-8.10 (m, 2H), 8.25 (br d, 1H), 8.33-8.52 (m, 3H).

[α]²⁰_{D}: +33° (c=1) in DMSO

### Example 857

### (6R)-6-{[7-bromo-2-(1-methyl-1H-pyrazol-4-yl)[1,2,4]triazolo[1,5-c]quinazolin-5-yl]amino}-1-imino-1λ⁶,4-thiazepane-1,5-dione, Diastereomer 2

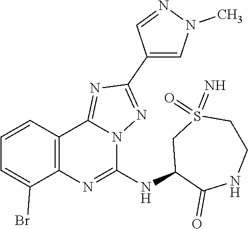 [SMILES: Cn1cc(-c2nc3c4cccc(Br)c4nc(N[C@H]4CS(=N)(=O)CCNC4=O)n3n2)cn1]

The title compound was isolated along with example 856 in the reaction described previously in 12% yield (2.6 mg)

¹H-NMR (400 MHz, DMSO-d₆): δ [ppm]=3.15-3.22 (m, 1H), 3.26 (br d, 1H), 3.48-3.57 (m, 1H), 3.62-3.82 (m, 4H), 3.96 (s, 3H), 5.26 (br d, 1H), 7.37 (t, 1H), 8.07 (s, 1H), 8.09 (dd, 1H), 8.17 (br s, 1H), 8.28 (dd, 1H), 8.46-8.52 (m, 2H).

[α]²⁰_{D}: +10° (c=1) in DMSO

### Example 858

### (3R)-3-{[7-(methanesulfonyl)-2-(1-methyl-1H-pyrazol-4-yl)[1,2,4]triazolo[1,5-c]quinazolin-5-yl]amino}azepan-2-one

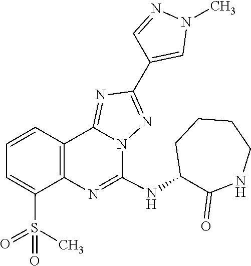 [SMILES: Cn1cc(-c2nc3c4cccc(S(C)(=O)=O)c4nc(N[C@@H]4CCCCNC4=O)n3n2)cn1]

(3R)-3-{[7-Bromo-2-(1-methyl-1H-pyrazol-4-yl)[1,2,4]triazolo[1,5-c]quinazolin-5-yl]amino}azepan-2-one (100 mg, 220 μmol), sodium methanesulfinate (33.6 mg, 329 μmol) and the copper(I) trifluoromethanesulfonate benzene complex (11.1 mg, 22.0 μmol]) were suspended in DMSO (500 μL) and trans-N,N′-dimethylcyclohexane-1,2-diamine (12.5 mg, 87.9 μmol) was added. The reaction was stirred for 6 h at 100° C. 0.5 mL of DMSO were added and the reaction was stirred 12 h at 100° C. The reaction was then stirred 24 h at 130° C. Sodium methanesulfinate (33.6 mg, 329 μmol), copper(I) trifluoromethanesulfonate benzene complex (11.1 mg, 22.0 μmol) and trans-N,N′-dimethylcyclohexane-1,2-diamine (12.5 mg, 87.9 μmol) were added and the reaction was stirred 5 h at 130° C. The reaction was cooled to rt and purified by preparative HPLC to give 25.0 mg (100% purity, 25% yield) of the title compound.

LC-MS (method 2): Rₜ=0.85 min; MS (ESIpos): m/z=455 [M+H]⁺

### Example 859

### (3R)-3-{[7-(2-hydroxypropan-2-yl)-2-(4-methoxyphenyl)[1,2,4]triazolo[1,5-c]quinazolin-5-yl]amino}azepan-2-one

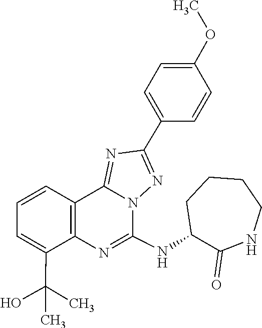 [SMILES: COc1ccc(-c2nc3c4cccc(C(C)(C)O)c4nc(N[C@@H]4CCCCNC4=O)n3n2)cc1]

Bromido(methyl)magnesium (340 μL, 3.2 M in Me-THF, 1.1 mmol) was dissolved in dry THF (1.5 mL) and the reaction was cooled to 0° C. and a solution of methyl 2-(4-methoxyphenyl)-5-{[(3R)-2-oxoazepan-3-yl]amino}[1,2,4]triazolo[1,5-c]quinazoline-7-carboxylate (50.0 mg, 109 μmol, example 250) in dry THF (1.5 mL) was added dropwise. The mixture allowed to warm up to rt and stirred overnight. The reaction mixture was diluted with ethyl acetate and the organic layer was washed with water. The organic layer was dried (silicon filter) and concentrated under reduced pressure to give 15.2 mg (93% purity, 28% yield) of the title compound without further purification.

LC-MS (method 2): Rₜ=1.28 min; MS (ESIneg): m/z=459 [M−H]⁻.

¹H-NMR (400 MHz, DMSO-d6): δ [ppm]=1.27-1.40 (m, 1H), 1.57-1.69 (m, 1H), 1.75 (s, 3H), 1.79 (s, 3H), 1.82-1.96 (m, 2H), 1.99-2.10 (m, 1H), 2.28-2.37 (m, 1H), 3.14-3.31 (m, 2H), 3.86 (s, 3H), 4.77 (br dd, 1H), 5.93 (s, 1H), 7.15 (d, 2H), 7.42 (t, 1H), 7.80 (d, 1H), 7.96 (dd, 1H), 8.18-8.29 (m, 4H).

### Example 860

### (3R)-3-{[7-(hydroxymethyl)-2-(4-methoxyphenyl)[1,2,4]triazolo[1,5-c]quinazolin-5-yl]amino}azepan-2-one

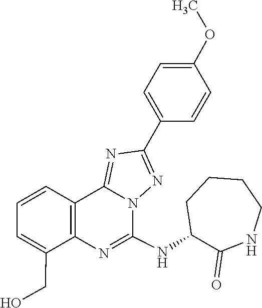 [SMILES: COc1ccc(-c2nc3c4cccc(CO)c4nc(N[C@@H]4CCCCNC4=O)n3n2)cc1]

Methyl 2-(4-methoxyphenyl)-5-{[(3R)-2-oxoazepan-3-yl]amino}[1,2,4]triazolo[1,5-c]quinazoline-7-carboxylate (350 mg, 760 μmol) was dissolved in dry THF (42 mL) and the reaction vessel was purged three times with argon. The reaction mixture was cooled to 0° C. and lithium borohydride (3.8 mL, 2.0 M, 7.6 mmol) was added dropwise. The mixture was stirred overnight at rt. The mixture was cooled to 0° C., lithium borohydride (3.8 mL, 2.0 M, 7.6 mmol) was added and the mixture was stirred 24 h at rt. The mixture was quenched with half saturated aqueous sodium hydrogen carbonate solution. The solvent was evaporated and the organic phase was extracted with dichloromethane. The organic layer was dried over a silicone filter and concentrated under reduced pressure. The crude mixture was purified by flash column chromatography to give 106 mg (82% purity, 26% yield) of the title compound

LC-MS (method 2): Rₜ=1.15 min; MS (ESIpos): m/z=433 [M+H]⁺

¹H-NMR (400 MHz, DMSO-d6): δ [ppm]=1.27-1.41 (m, 1H), 1.48-1.60 (m, 1H), 1.81-1.95 (m, 2H), 2.06 (br dd, 1H), 2.37 (br d, 1H), 3.13-3.23 (m, 1H), 3.36-3.43 (m, 1H), 3.86 (s, 3H), 4.82 (br dd, 1H), 4.95-5.02 (m, 1H), 5.03-5.11 (m, 1H), 5.26 (t, 1H), 7.11-7.17 (m, 2H), 7.46 (t, 1H), 7.66 (d, 1H), 7.82-7.87 (m, 1H), 8.18 (dd, 1H), 8.20-8.25 (m, 3H).

### Example 861

### (3R)-3-{[7-(2-hydroxypropan-2-yl)-2-(1-methyl-1H-pyrazol-4-yl)[1,2,4]triazolo[1,5-c]quinazolin-5-yl]amino}azepan-2-one

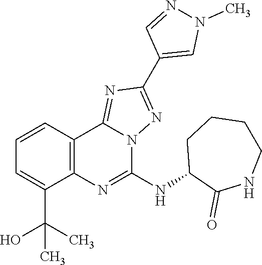 [SMILES: Cn1cc(-c2nc3c4cccc(C(C)(C)O)c4nc(N[C@@H]4CCCCNC4=O)n3n2)cn1]

Bromido(methyl)magnesium (830 μL, 3.2 M, 2.6 mmol) was dissolved in dry THF (5.3 mL) and the mixture was cooled to 0° C. A solution of methyl 2-(1-methyl-1H-pyrazol-4-yl)-5-{[(3R)-2-oxoazepan-3-yl]amino}[1,2,4]triazolo[1,5-c]quinazoline-7-carboxylate (115 mg, 264 μmol) in dry THF (2 mL) was added dropwise. The mixture was warmed slowly to rt and stirred overnight. The mixture was diluted with ethyl acetate and washed with aqueous saturated ammonium chloride solution. The organic layer was dried over a silicone filter and concentrated under reduced pressure. The crude material was purified by preparative HPLC to give 55.5 mg (100% purity, 48% yield) of the title compound.

LC-MS (method 2): Rₜ=1.00 min; MS (ESIpos): m/z=435 [M+H]⁺

¹H-NMR (400 MHz, DMSO-d6): δ [ppm]=1.26-1.39 (m, 1H), 1.55-1.68 (m, 1H), 1.74 (s, 3H), 1.79 (s, 3H), 1.83-1.96 (m, 2H), 1.98-2.09 (m, 1H), 2.27-2.35 (m, 1H), 3.14-3.30 (m, 2H), 3.95 (s, 3H), 4.75 (dd, 1H), 5.94 (s, 1H), 7.41 (t, 1H), 7.70 (d, 1H), 7.94 (dd, 1H), 8.07 (s, 1H), 8.17 (dd, 1H), 8.26 (dd, 1H), 8.49 (s, 1H).

### Example 862

### (3R)-3-{[10-(hydroxymethyl)-2-(4-methoxyphenyl)[1,2,4]triazolo[1,5-c]quinazolin-5-yl]amino}azepan-2-one

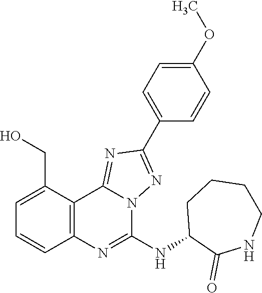 [SMILES: COc1ccc(-c2nc3c4c(CO)cccc4nc(N[C@@H]4CCCCNC4=O)n3n2)cc1]

Methyl 2-(4-methoxyphenyl)-5-{[(3R)-2-oxoazepan-3-yl]amino}[1,2,4]triazolo[1,5-c]quinazoline-10-carboxylate (136 mg, 295 μmol) was solubilised in dry THF (10 mL), and the flask was purged with argon. The reaction mixture was cooled to 0° C. and lithium borohydride (1.5 mL, 2.0 M in THF, 3.0 mmol) was added dropwise. The mixture was stirred overnight at rt. The reaction was quenched with half saturated aqueous sodium hydrogen carbonate solution and the solvent was evaporated. The aqueous phase was extracted with dichloromethane saturated aqueous and the organic phase was died (sodium sulfate), filtered and concentrated under reduced pressure. The crude mixture was purified by preparative HPLC to give 8.00 mg (90% purity, 6% yield) of the title compound.

LC-MS (method 2): Rₜ=1.23 min; MS (ESIpos): m/z=433 [M+H]⁺

¹H-NMR (400 MHz, DMSO-d6) δ[ppm]: 1.304 (0.51), 1.338 (1.75), 1.547 (0.54), 1.578 (0.59), 1.832 (0.62), 1.839 (0.66), 1.865 (0.94), 1.889 (0.52), 1.898 (0.51), 2.006 (0.61), 2.303 (0.61), 2.327 (0.55), 2.332 (0.61), 2.522 (0.67), 2.539 (3.78), 3.165 (0.44), 3.183 (0.46), 3.321 (0.63), 3.385 (0.50), 3.853 (16.00), 4.805 (0.57), 4.817 (0.61), 4.830 (0.60), 4.844 (0.57), 5.309 (2.42), 5.323 (2.72), 5.549 (1.16), 5.564 (2.38), 5.579 (0.94), 6.316 (0.49), 6.556 (3.04), 7.124 (0.51), 7.131 (3.89), 7.148 (1.45), 7.153 (4.04), 7.537 (1.41), 7.558 (1.79), 7.588 (1.17), 7.590 (1.19), 7.606 (1.67), 7.682 (2.62), 7.701 (2.91), 7.721 (0.98), 8.192 (4.86), 8.197 (1.96), 8.209 (2.25), 8.214 (4.50), 8.221 (1.26).

### Example 863

### (3R)-3-({2-(4-methoxyphenyl)-7-[(methylsulfanyl)methyl][1,2,4]triazolo[1,5-c]quinazolin-5-yl}amino)azepan-2-one

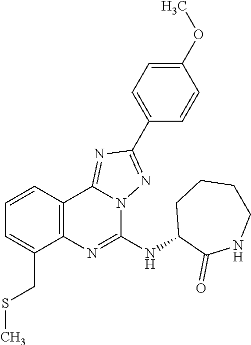 [SMILES: COc1ccc(-c2nc3c4cccc(CSC)c4nc(N[C@@H]4CCCCNC4=O)n3n2)cc1]

(3R)-3-{[7-(Chloromethyl)-2-(4-methoxyphenyl)[1,2,4]triazolo[1,5-c]quinazolin-5-yl]amino}azepan-2-one (13.0 mg, 28.8 μmol) and sodium methanethiolate (2.42 mg, 34.6 μmol) were dissolved in DMF (200 μL) and stirred for 1 h at 50° C. Water was added to the mixture and the solid was filtered. The solid was suspended in water, sonicated for 15 minutes and filtered again. The new solid was washed with water and dried under reduced pressure at 60° C. to give 9.00 mg (90% purity, 61% yield) of the title compound without further purification

LC-MS (method 2): Rₜ=1.43 min; MS (ESIpos): m/z=463 [M+H]⁺

¹H-NMR (400 MHz, DMSO-d6): δ [ppm]=1.27-1.41 (m, 1H), 1.48-1.62 (m, 1H), 1.81-1.98 (m, 2H), 1.99-2.10 (m, 4H), 2.40-2.46 (m, 1H), 3.14-3.25 (m, 1H), 3.38-3.42 (m, 1H), 3.86 (s, 3H), 3.99 (d, 1H), 4.33 (d, 1H), 4.83 (br dd, 1H), 7.14 (d, 2H), 7.41 (t, 1H), 7.65-7.75 (m, 2H), 8.22 (br d, 4H).

### Example 864

### ethyl N-[2-(1-methyl-1H-pyrazol-4-yl)-7-(trifluoromethyl)[1,2,4]triazolo[1,5-c]quinazolin-5-yl]-D-alaninate

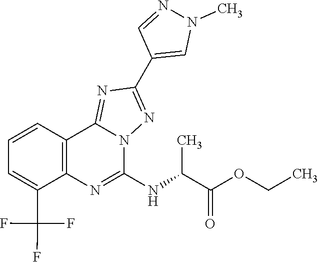 [SMILES: CCOC(=O)[C@@H](C)Nc1nc2c(C(F)(F)F)cccc2c2nc(-c3cnn(C)c3)nn12]

To a solution of 5-chloro-2-(1-methyl-1H-pyrazol-4-yl)-7-(trifluoromethyl)[1,2,4]triazolo[1,5-c]quinazoline (640 mg, 1.81 mmol) and ethyl D-alaninate-hydrogen chloride (1/1) (585 mg, 3.81 mmol) was added N,N-diisopropylethylamine (1.4 mL, 8.2 mmol). This mixture was stirred in DMSO (10 mL) for 1 h at 60° C. After cooling to rt water was added to the mixture, filtered, and washed with water. Then the solid was dissolved in ethyl acetate and this organic phase was washed two times with water, brine, filtered using a hydrophobic phase separation filter paper and concentrated under reduced pressure to give 630 mg (92% purity, 74% yield) of the target compound, which was used without further purification.

LC-MS (Method 1): Rₜ=1.32 min; MS (ESIpos): m/z=434 [M+H]⁺

¹H-NMR (400 MHz, DMSO-d₆): δ [ppm]=1.18 (t, 3H), 1.62 (d, 3H), 3.97 (s, 3H), 4.01-4.17 (m, 2H), 4.66-4.73 (m, 1H), 7.53 (t, 1H), 8.04-8.09 (m, 2H), 8.44 (s, 1H), 8.49 (dd, 1H), 8.77 (br s, 1H).

### Example 865

### ethyl N-[7-bromo-2-(4-methoxyphenyl)[1,2,4]triazolo[1,5-c]quinazolin-5-yl]-D-alaninate

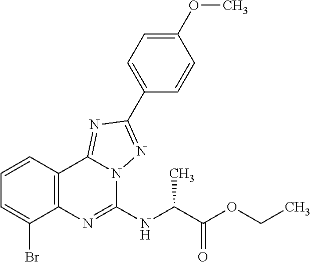 [SMILES: CCOC(=O)[C@@H](C)Nc1nc2c(Br)cccc2c2nc(-c3ccc(OC)cc3)nn12]

7-Bromo-5-chloro-2-(4-methoxyphenyl)[1,2,4]triazolo[1,5-c]quinazoline (1.00 g, 2.57 mmol) was solubilised in DMSO (10.3 mL), N,N-diisopropylethylamine (1.34 mL, 7.7 mmol) and ethyl D-alaninate-hydrogen chloride (1/1) (622 mg, 95% purity, 3.85 mmol) were added and the mixture was stirred at 60° C. for 1 h. The mixture was allowed to reach rt, poured into water and stirred for 30 minutes. The precipitate was filtered off, washed twice with water dried under reduced pressure to afford 1.10 g of the target compound, which was used without further purification in the next step.

LC-MS (Method 2): Rₜ=1.57 min; MS (ESIpos): m/z=470 [M+H]⁺

¹H-NMR (400 MHz, DMSO-d₆): δ [ppm]=1.20 (t, 3H), 1.64 (d, 3H), 3.86 (s, 3H), 4.16 (q, 2H), 4.79 (quin, 1H), 7.14-7.19 (m, 2H), 7.34 (t, 1H), 8.05 (dd, 1H), 8.24-8.28 (m, 2H), 8.30 (dd1H), 8.72 (d, 1H).

### Example 866

### ethyl N-[7-bromo-2-(1-methyl-1H-pyrazol-4-yl)[1,2,4]triazolo[1,5-c]quinazolin-5-yl]-D-alaninate

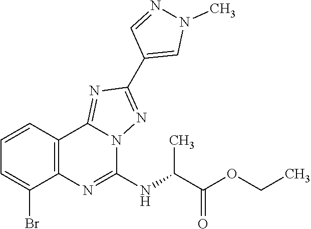 [SMILES: CCOC(=O)[C@@H](C)Nc1nc2c(Br)cccc2c2nc(-c3cnn(C)c3)nn12]

7-Bromo-5-chloro-2-(1-methyl-1H-pyrazol-4-yl)[1,2,4]triazolo[1,5-c]quinazoline (1.20 g, 3.30 mmol) was solubilised in DMSO (12 mL), N,N-diisopropylethylamine (1.73 mL, 9.9 mmol) and ethyl D-alaninate-hydrogen chloride (1/1) (760 mg, 4.95 mmol) were added and the mixture was stirred at 60° C. for 1 h. The mixture was allowed to reach rt, poured into water (100 mL) and stirred for 30 minutes. The precipitate was filtered off, washed twice with water dried under reduced pressure obtaining 1.025 g (70%) of the target compound, which was used without further purification in the next step.

LC-MS (Method 2): Rₜ=1.29 min; MS (ESIpos): m/z=444 [M+H]⁺

¹H-NMR (400 MHz, DMSO-d₆): δ [ppm]=1.19 (t, 3H), 1.63 (d, 3H), 3.97 (s, 3H), 4.15 (q, 2H), 4.77 (quin, 1H), 7.32 (t, 1H), 8.04 (dd, 1H), 8.07 (d, 1H), 8.24 (dd, 1H), 8.44 (s, 1H), 8.67 (d, 1H).

### Example 867

### methyl N-[7-bromo-2-(4-methoxyphenyl)[1,2,4]triazolo[1,5-c]quinazolin-5-yl]-D-valinate

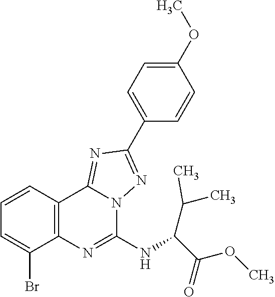 [SMILES: COC(=O)[C@H](Nc1nc2c(Br)cccc2c2nc(-c3ccc(OC)cc3)nn12)C(C)C]

7-Bromo-5-chloro-2-(4-methoxyphenyl)[1,2,4]triazolo[1,5-c]quinazoline (400 mg, 1.03 mmol) and (2R)-1-methoxy-3-methyl-1-oxobutan-2-aminium chloride (258 mg, 1.54 mmol) were stirred in DMSO (7.6 mL), N,N-diisopropylethylamine (720 μL, 4.1 mmol) was added and the reaction was stirred for 3 h at 60° C. The reaction mixture was cooled to rt, poured into water, and stirred overnight. The precipitate were filtered, the solid was washed with water and dried under reduced pressure at 60° C. Crude product was treated with diethylether and sonicated. The solid was filtered and dried under reduced pressure to give 479 mg (95% purity, 92% yield) of the title compound without further purification.

LC-MS (method 2): Rₜ=1.36 min; MS (ESIpos): m/z=486 [M+H]⁺

1H-NMR (400 MHz, DMSO-d6): δ [ppm]=1.06 (d, 3H), 1.11 (d, 3H), 3.73 (s, 3H), 3.86 (s, 3H), 4.64 (t, 1H), 7.12-7.19 (m, 2H), 7.35 (t, 1H), 8.06 (dd, 1H), 8.18 (br d, 1H), 8.24-8.33 (m, 3H).

### Example 868

### methyl N-[7-bromo-2-(1-methyl-1H-pyrazol-4-yl)[1,2,4]triazolo[1,5-c]quinazolin-5-yl]-D-valinate

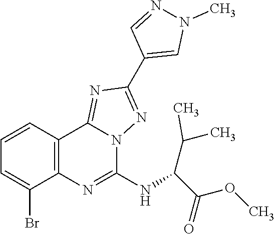 [SMILES: COC(=O)[C@H](Nc1nc2c(Br)cccc2c2nc(-c3cnn(C)c3)nn12)C(C)C]

7-Bromo-5-chloro-2-(1-methyl-1H-pyrazol-4-yl)[1,2,4]triazolo[1,5-c]quinazoline (400 mg, 1.10 mmol) and (2R)-1-methoxy-3-methyl-1-oxobutan-2-aminium chloride (277 mg, 1.65 mmol) were stirred in DMSO (8.1 mL), N,N-diisopropylethylamine (770 μL, 4.4 mmol) was added and the reaction was stirred 3 h at 60° C. The reaction mixture was cooled to rt, poured into water and stirred overnight. The solid was filtered, washed with water and dried under reduced pressure at 60° C. The crude product was treated with diethylether and sonicated. The solid was filtered and dried under reduced pressure to give 370 mg (90% purity, 66% yield) of the title compound without further purification.

LC-MS (method 2): Rₜ=1.36 min; MS (ESIneg): m/z=456 [M−H]⁻.

1H-NMR (400 MHz, DMSO-d6): δ [ppm]=1.04 (d, 3H), 1.09 (d, 3H), 2.42-2.48 (m, 1H), 3.72 (s, 3H), 3.96 (s, 3H), 4.63 (t, 1H), 7.33 (t, 1H), 8.01-8.11 (m, 3H), 8.24 (dd, 1H), 8.48 (s, 1H).

### Example 869

### N-[2-(1-methyl-1H-pyrazol-4-yl)-7-(trifluoromethyl)[1,2,4]triazolo[1,5-c]quinazolin-5-yl]-D-alanine

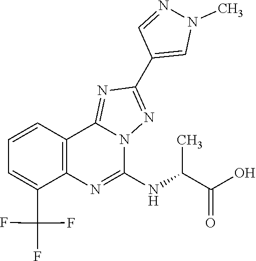 [SMILES: C[C@@H](Nc1nc2c(C(F)(F)F)cccc2c2nc(-c3cnn(C)c3)nn12)C(=O)O]

To a suspension of ethyl N-[2-(1-methyl-1H-pyrazol-4-yl)-7-(trifluoromethyl)[1,2,4]triazolo[1,5-c]quinazolin-5-yl]-D-alaninate (380 mg, 877 μmol) in ethanol (4.6 mL) was added an aqueous 2 M sodium hydroxide solution (1.8 mL) and this mixture was stirred for 1 h at rt. Then the reaction mixture was concentrated under reduced pressure and dissolved in water (10 mL). To this stirred aqueous solution 10% aqueous sulfuric acid was added up to acidic pH. The formed solid was collected via filtration, dried to give 316 mg (100% purity, 89% yield) of the target compound, which was used without further purification.

LC-MS (Method 2): Rₜ=0.69 min; MS (ESIpos): m/z=406 [M+H]⁺

¹H-NMR (400 MHz, DMSO-d₆): δ [ppm]=1.61 (d, 3H), 3.97 (s, 3H), 4.74 (dq, 1H), 7.53 (t, 1H), 8.05-8.09 (m, 2H), 8.44-8.48 (m, 2H), 8.49 (dd, 1H), 12.77 (br s, 1H).

### Example 870

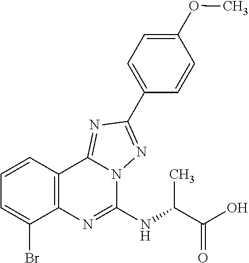 [SMILES: COc1ccc(-c2nc3c4cccc(Br)c4nc(N[C@H](C)C(=O)O)n3n2)cc1]

Ethyl N-[7-bromo-2-(4-methoxyphenyl)[1,2,4]triazolo[1,5-c]quinazolin-5-yl]-D-alaninate (1.56 g, 3.32 mmol) was suspended in ethanol (17.3 mL), NaOH (6.6 mL, 2.0 M, 13.3 mmol) was added and stirred for 1 h at rt. The mixture was evaporated, diluted with water (100 mL), acidified with hydrogen chloride (2.0 M) to pH 3-4 and the resulting precipitate was filtered off, washed with water and dried under reduced pressure at 50° C. to yield 1.54 g of the target compound, which was used without further purification in the next step.

LC-MS (Method 2): Rₜ=0.80 min; MS (ESIpos): m/z=442 [M+H]⁺

1H-NMR (400 MHz, DMSO-d6): δ [ppm]=1.65 (d, 3H), 3.86 (s, 3H), 4.84 (quin, 1H), 7.16 (d, 2H), 7.33 (t, 1H), 8.05 (dd, 1H), 8.26 (d, 2H), 8.30 (dd, 1H), 8.47 (d, 1H), 12.87 (br s, 1H).

### Example 871

### N-[7-bromo-2-(1-methyl-1H-pyrazol-4-yl)[1,2,4]triazolo[1,5-c]quinazolin-5-yl]-D-alanine

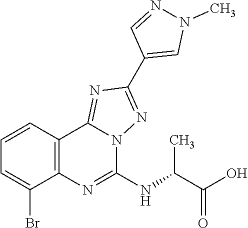 [SMILES: C[C@@H](Nc1nc2c(Br)cccc2c2nc(-c3cnn(C)c3)nn12)C(=O)O]

Ethyl N-[7-bromo-2-(1-methyl-1H-pyrazol-4-yl)[1,2,4]triazolo[1,5-c]quinazolin-5-yl]-D-alaninate (1.96 g, 4.40 mmol) was suspended in ethanol (23 mL), NaOH (8.8 mL, 2.0 M, 17.6 mmol) was added and stirred for 1 h at rt. The mixture was evaporated, diluted with water (100 mL), acidified with hydrogen chloride (2.0 M) to pH 3-4 and the resulting precipitate was filtered, washed three times with water and dried under reduced pressure at 50° C. to give 1.81 g (99%) of the target compound, which was used without further purification in the next step.

LC-MS (Method 2): Rₜ=0.68 min; MS (ESIpos): m/z=416 [M+H]⁺

¹H-NMR (400 MHz, DMSO-d₆): δ [ppm]=1.63 (d, 3H), 3.96 (s, 3H), 4.82 (quin, 1H), 7.32 (t, 1H), 8.04 (dd, 1H), 8.07 (s, 1H), 8.24 (dd, 1H), 8.37 (d, 1H), 8.45 (s, 1H), 12.87 (br s, 1H).

### Example 872

### N-[7-bromo-2-(4-methoxyphenyl)[1,2,4]triazolo[1,5-c]quinazolin-5-yl]-D-valine

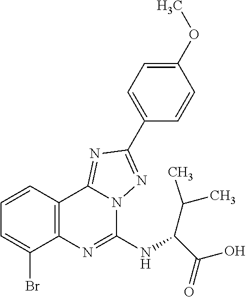 [SMILES: COc1ccc(-c2nc3c4cccc(Br)c4nc(N[C@@H](C(=O)O)C(C)C)n3n2)cc1]

Methyl N-[7-bromo-2-(4-methoxyphenyl)[1,2,4]triazolo[1,5-c]quinazolin-5-yl]-D-valinate (470 mg, 970 μmol) was solubilised in THF (19 mL)/methanol (6.5 mL), aqueous lithium hydroxide solution (5.8 mL, 1.0 M, 5.8 mmol) was added and the reaction was stirred at rt overnight. Aqueous citric acid (5.9 mL, 1.0 M, 5.9 mmol) was added and stirred the reaction was stirred for 1 h. The reaction was diluted with water and the organic solvents were removed under reduced pressure. The aqueous phase was filtered, the solid was washed with water and dried under reduced pressure at 60° C. to give 407 mg (95% purity, 85% yield) of the title compound without further purification.

LC-MS (method 2): Rₜ=0.84 min; MS (ESIpos): m/z=472 [M+H]⁺

1H-NMR (400 MHz, DMSO-d6): δ [ppm]=1.09 (d, 6H), 3.86 (s, 3H), 4.77 (dd, 1H), 7.11-7.18 (m, 2H), 7.35 (t, 1H), 7.65 (d, 1H), 8.06 (dd, 1H), 8.23-8.34 (m, 3H), 12.55-13.87 (m, 1H).

### Example 873

### N-[7-bromo-2-(1-methyl-1H-pyrazol-4-yl)[1,2,4]triazolo[1,5-c]quinazolin-5-yl]-D-valine

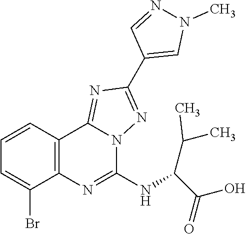 [SMILES: CC(C)[C@@H](Nc1nc2c(Br)cccc2c2nc(-c3cnn(C)c3)nn12)C(=O)O]

Methyl N-[7-bromo-2-(1-methyl-1H-pyrazol-4-yl)[1,2,4]triazolo[1,5-c]quinazolin-5-yl]-D-valinate (362 mg, 790 μmol) was solubilised in THF (15 mL)/methanol (5.3 mL) and lithium hydroxide (4.7 mL, 1.0 M, 4.7 mmol) was added. The reaction was stirred overnight at rt. Aqueous citric acid (4.8 mL, 1.0 M, 4.8 mmol) was added and the reaction was stirred for 1 h. The reaction mixture was diluted with water and the organic solvents were removed under reduced pressure. The aqueous phase was filtered, the solid was washed with water and dried under reduced pressure at 60° C. to give 332 mg (95% purity, 90% yield) of the title compound without further purification.

LC-MS (method 2): Rₜ=0.69 min; MS (ESIneg): m/z=442 [M−H]⁻.

1H-NMR (400 MHz, DMSO-d6): δ [ppm]=1.07 (d, 6H), 2.41-2.47 (m, 1H), 3.96 (s, 3H), 4.76 (dd, 1H), 7.33 (t, 1H), 7.49 (d, 1H), 8.05 (dd, 1H), 8.10 (s, 1H), 8.24 (dd, 1H), 8.50 (s, 1H), 12.45-13.67 (m, 1H).

### Example 874

### (2R)-2-{[2-(1-methyl-1H-pyrazol-4-yl)-7-(trifluoromethyl)[1,2,4]triazolo[1,5-c]quinazolin-5-yl]amino}-1-(morpholin-4-yl)propan-1-one

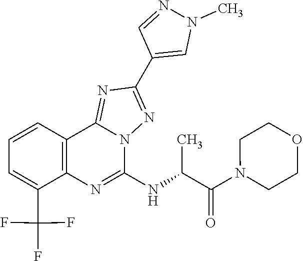 [SMILES: C[C@@H](Nc1nc2c(C(F)(F)F)cccc2c2nc(-c3cnn(C)c3)nn12)C(=O)N1CCOCC1]

To a solution of N-[2-(1-methyl-1H-pyrazol-4-yl)-7-(trifluoromethyl)[1,2,4]triazolo[1,5-c]quinazolin-5-yl]-D-alanine (75.0 mg, 185 μmol) in DMF was added morpholine (32 μL, 370 μmol; CAS [110-91-8]), N,N-diisopropylethylamine (150 μL, 830 μmol; CAS [7087-68-5]) and 50% solution of propylphosphonic anhydride in ethyl acetate (160 μL, 50% purity, 280 μmol; CAS [68957-94-8]). This solution was stirred for 1 h at rt, concentrated under reduced pressure and the residue was purified by preparative HPLC under basic conditions to give 27.2 mg (95% purity, 29% yield) of the target compound

LC-MS (Method 2): Rₜ=1.08 min; MS (ESIneg): m/z=473 [M−H]⁻

¹H-NMR (400 MHz, DMSO-d₆): δ [ppm]=1.52 (d, 3H), 3.50-3.78 (m, 8H), 3.97 (s, 3H), 5.10 (dq, 1H), 7.53 (t, 1H), 8.07-8.10 (m, 2H), 8.24 (d, 1H), 8.47 (s, 1H), 8.50 (dd, 1H).

The following examples were prepared similarly to example 874.

| | Structure |
|---|---|
| | IUPAC-Name |
| Ex- | LC-MS (method): Retention time; Mass found |
| ample | ¹H-NMR |
| Ex- ample 875 | 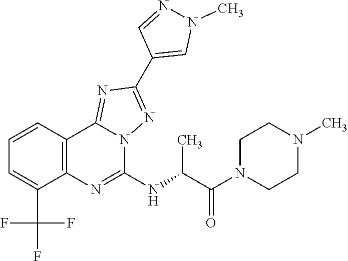 [SMILES: C[C@@H](Nc1nc2c(C(F)(F)F)cccc2c2nc(-c3cnn(C)c3)nn12)C(=O)N1CCN(C)CC1] |
| | (2R)-1-(4-methylpiperazin-1-yl)-2-{[2-(1-methyl-1H-pyrazol-4-yl)- |
| | 7-(trifluoromethyl)[1,2,4]triazolo[1,5-c]quinazolin-5- |
| | yl]amino}propan-1-one |
| | LC-MS (Method 2): Rₜ = 1.11 min; MS (ESIneg): m/z = 486 [M − H]⁻ |
| | ¹H-NMR (400 MHz, DMSO-d₆): δ [ppm] = 1.51 (d, 3H), 2.15-2.23 |
| | (m, 4H), 2.26-2.40 (m, 2H), 2.45-2.56 (m, 1H, partial in |
| | DMSO signal), 3.28-3.36 |
| | (m, 1H, partial in water signal), 3.48-3.57 (m, 1H), 3.64 (br s, 2H), |
| | 3.96 (s, 3H), 5.12 (dq, 1H), 7.53 (t, 1H), 8.07 (dd, 1H), 8.08 (d, 1H), |
| | 8.18 (d, 1H), 8.47 (d, 1H), 8.49 (dd, 1H). |
| Ex- ample 876 | 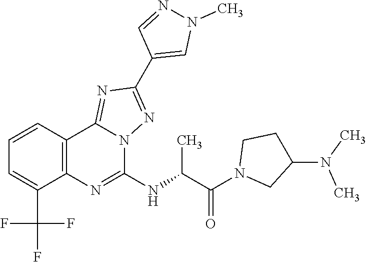 [SMILES: C[C@@H](Nc1nc2c(C(F)(F)F)cccc2c2nc(-c3cnn(C)c3)nn12)C(=O)N1CCC(N(C)C)C1] |
| | (2R)-1-[(3R/S)-3-(dimethylamino)pyrrolidin-1-yl]-2-{[2-(1-methyl-1H- |
| | pyrazol-4-yl)-7-(trifluoromethyl)[1,2,4]tri- |
| | azolo[1,5-c]quinazolin-5-yl]amino}propan-1-one |
| | LC-MS (Method 2): Rₜ = 1.11 min; MS (ESIpos): m/z = 502 [M + H]⁺ |
| | ¹H-NMR (400 MHz, DMSO-d₆): δ [ppm] = 1.47-1.55 (m, |
| | 3H), 1.56-1.92 (m, 1H), 2.01-2.24 (m, |
| | 7H), 2.55-3.04 (m, 1H), 3.15-3.43 (m, 1H, partial |
| | in water signal), 3.48-3.83 (m, 2H), 3.88-4.07 (m, 1H), 3.96 (s, 3H), |
| | 4.78-4.97 (m, 1H), 7.48-7.56 (m, 1H), 8.02-8.09 (m, 2H), 8.17 (br s, |
| | 1H), 8.43-8.46 (m, 1H), 8.48 (dd, 1H). |
| Ex- ample 877 | 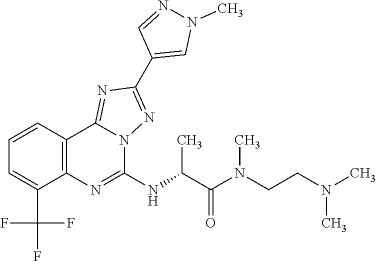 [SMILES: C[C@@H](Nc1nc2c(C(F)(F)F)cccc2c2nc(-c3cnn(C)c3)nn12)C(=O)N(C)CCN(C)C] |
| | N-[2-(dimethylamino)ethyl]-N-methyl-N²-[2-(1-methyl- |
| | 1H-pyrazol-4-yl)-7- |
| | (trifluoromethyl)[1,2,4]triazolo[1,5-c]quinazolin-5-yl]-D-alaninamide |
| | LC-MS (Method 2): Rₜ = 1.18 min; MS (ESIpos): m/z = 490 [M + H]⁺ |
| | ¹H-NMR (400 MHz, DMSO-d₆): δ [ppm] = 1.47-1.55 (m, 3H), 2.11- |
| | 2.17 (m, 6H), 2.27-2.41 (m, 2H), 2.91 and 3.16 (2s, 3H), 3.23- |
| | 3.32 (m, 1H), 3.40-3.58 (m, 1H), 3.96 (s, 3H), 5.05-5.23 (m, 1H), |
| | 7.50-7.56 (m, 1H), 8.04-8.11 (m, 3H), 8.45-8.51 (m, 2H). |
| Ex- ample 878 | 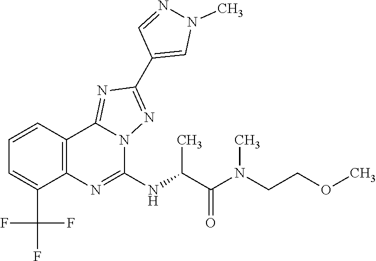 [SMILES: COCCN(C)C(=O)[C@@H](C)Nc1nc2c(C(F)(F)F)cccc2c2nc(-c3cnn(C)c3)nn12] |
| | N-(2-methoxyethyl)-N-methyl-N²-[2-(1-methyl-1H-pyrazol-4-yl)-7- |
| | (trifluoromethyl)[1,2,4]triazolo[1,5-c]quinazolin-5-yl]-D-alaninamide |
| | LC-MS (Method 2): Rₜ = 1.20 min; MS (ESIneg): m/z = 475 [M − H]⁻ |
| | ¹H-NMR (400 MHz, DMSO-d₆): δ [ppm] = 1.48-1.55 (m, 3H), 2.89 |
| | and 3.18 (2s, 3H), 3.23 and 3.26 (2s, 3H), 3.34-3.82 (m, 4H), |
| | 3.96 (s, 3H), 5.12 and 5.23 (2quin, 1H), 7.53 (t, 1H), |
| | 8.04-8.15 (m, 3H), 8.45-8.51 (m, 2H). |
| Ex- ample 879 | 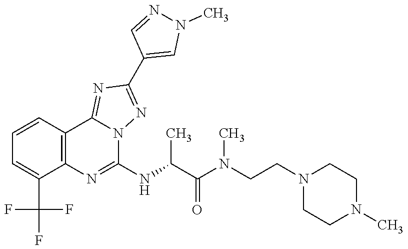 [SMILES: C[C@@H](Nc1nc2c(C(F)(F)F)cccc2c2nc(-c3cnn(C)c3)nn12)C(=O)N(C)CCN1CCN(C)CC1] |
| | N-[2-(4-methylpiperazin-1-yl)ethyl]-N²-[2-(1-methyl-1H- |
| | pyrazol-4-yl)-7- |
| | (trifluoromethyl)[1,2,4]triazolo[1,5-c]quinazolin-5-yl]-D-alaninamide |
| | LC-MS (Method 2): Rₜ = 0.80 min; MS (ESIpos): m/z = 531 [M + H]⁺ |
| | ¹H-NMR (400 MHz, DMSO-d₆): δ [ppm] = 1.54 (d, 3H), 1.99-2.18 |
| | (m, 4H), 2.02 (s, 3H), 2.22-2.37 (m, 6H), 3.11-3.27 (m, 2H), 3.96 (s, |
| | 3H), 4.69 (q, 1H), 7.53 (t, 1H), 8.00 (br t, 1H), 8.06-8.10 (m, |
| | 2H), 8.48 (s, 1H), 8.50 (dd, 1H). |
| Ex- ample 880 | 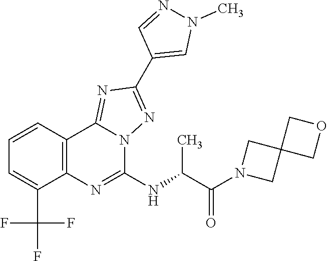 [SMILES: C[C@@H](Nc1nc2c(C(F)(F)F)cccc2c2nc(-c3cnn(C)c3)nn12)C(=O)N1CC2(COC2)C1] |
| | (2R)-2-{[2-(1-methyl-1H-pyrazol-4-yl)-7- |
| | (trifluoromethyl)[1,2,4]triazolo[1,5-c]quinazolin-5-yl]amino}-1-(2- |
| | oxa-6-azaspiro[3.3]heptan-6-yl)propan-1-one |
| | LC-MS (Method 2): Rₜ = 0.80 min; MS (ESIneg): |
| | m/z = 485 [M − H]⁻ |
| | ¹H-NMR (400 MHz, DMSO-d₆): δ [ppm] = 1.48 (d, 3H), |
| | 3.96 (s, 3H), 4.01-4.13 (m, 2H), 4.44-4.54 (m, 2H), 4.62-4.77 (m, |
| | 5H), 7.53 (t, 1H), 8.06-8.09 (m, 2H), 8.28 (br d, 1H), |
| | 8.45 (s, 1H), 8.49 (dd, 1H). |

### Example 881

### N²-[2-(4-methoxyphenyl)[1,2,4]triazolo[1,5-c]quinazolin-5-yl]-N-[2-(4-methylpiperazin-1-yl)ethyl]-D-alaninamide

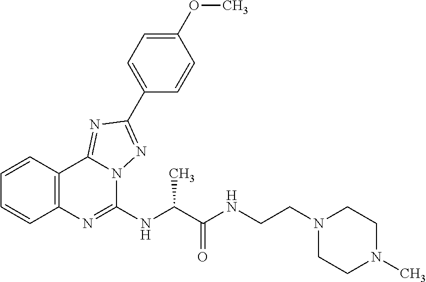 [SMILES: COc1ccc(-c2nc3c4ccccc4nc(N[C@H](C)C(=O)NCCN4CCN(C)CC4)n3n2)cc1]

N-[2-(4-Methoxyphenyl)[1,2,4]triazolo[1,5-c]quinazolin-5-yl]-D-alanine (85.0 mg, 234 μmol) was dissolved in DMF (2.3 mL). 2-(4-Methylpiperazin-1-yl)ethan-1-amine (67.0 mg, 468 μmol), N,N-diisopropylethylamine (180 μL, 1.1 mmol), and 2,4,6-tripropyl-1,3,5,2,4,6-trioxatriphosphinane 2,4,6-trioxide (200 μL, 50% purity, 350 μmol) were added. It was stirred for 1.5 h at rt. The reaction mixture was purified by HPLC. The product was dissolved in dichloromethane and saturated aqueous sodium hydrogen carbonate solution was added. The mixture was stirred, the phases were separated and the organic layer was filtered over a hydrophobic filter and concentrated affording 23 mg (20%) of the title product.

LC-MS (Method 2): Rₜ=1.11 min; MS (ESIneg): m/z=487 [M−H]₋

¹H-NMR (400 MHz, DMSO-d₆): δ [ppm]=1.53 (d, 3H), 2.03 (s, 3H), 2.06-2.38 (m, 9H), 3.20 (q, 2H), 3.86 (s, 3H), 4.76 (quin, 1H), 7.12-7.17 (m, 2H), 7.41-7.46 (m, 1H), 7.62 (d, 1H), 7.72 (ddd, 1H), 7.84 (d, 1H), 8.02 (t, 1H), 8.24-8.27 (m, 2H), 8.29 (dd, 1H). one proton is missing

The following examples were prepared analogously to example 881.

| | Structure |
|---|---|
| | IUPAC-Name |
| | LC-MS (method): Retention time; Mass found |
| Example | ¹H-NMR |
| Example 882 | 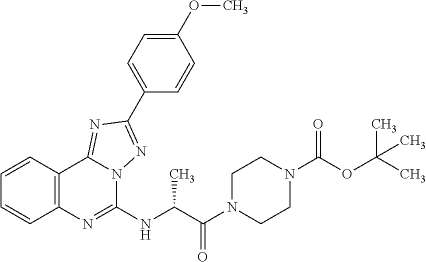 [SMILES: COc1ccc(-c2nc3c4ccccc4nc(N[C@H](C)C(=O)N4CCN(C(=O)OC(C)(C)C)CC4)n3n2)cc1] |
| | tert-butyl 4-{N-[2-(4-methoxyphenyl)[1,2,4]triazolo[1,5-c]quinazolin-5-yl]- |
| | D-alanyl}piperazine-1-carboxylate |
| | LC-MS (Method 2): Rt = 1.43 min; MS (ESIpos): m/z = 532 [M + H]⁺ |
| | ¹H-NMR (400 MHz, DMSO-d₆): δ [ppm] = 1.43 (s, 9H), 1.51 (d, 3H), 3.18-3.28 |
| | (m, 1H), 3.35-3.50 (m, 3H), 3.51-3.69 (m, 3H), 3.73-3.82 (m, 1H), 3.86 (s, |
| | 3H), 5.20 (quin, 1H), 7.12-7.17 (m, 2H), 7.45 (ddd, 1H), 7.62 (d, 1H), 7.73 |
| | (ddd, 1H), 7.92 (d, 1H), 8.23-8.27 (m, 2H), 8.29 (dd, 1H). |
| Example 883 | 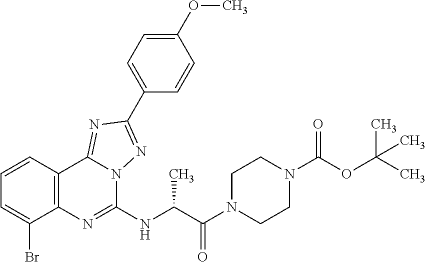 [SMILES: COc1ccc(-c2nc3c4cccc(Br)c4nc(N[C@H](C)C(=O)N4CCN(C(=O)OC(C)(C)C)CC4)n3n2)cc1] |
| | tert-butyl 4-{N-[7-bromo-2-(4-methoxyphenyl)[1,2,4]triazolo[1,5- |
| | c]quinazolin-5-yl]-D-alanyl}piperazine-1-carboxylate |
| | LC-MS (Method 2): Rt = 1.58 min; MS (ESIpos): m/z = 610 [M + H]⁺ |
| | ¹H-NMR (400 MHz, DMSO-d₆): δ [ppm] = 1.42 (s, 9H), 1.55 (d, 3H), 3.36-3.58 |
| | (m, 5H), 3.66-3.79 (m, 2H), 3.86 (s, 3H), 5.18-5.27 (m, 1H), 7.13-7.18 (m, |
| | 2H), 7.33 (t, 1H), 8.06 (dd, 1H), 8.23-8.31 (m, 4H). one proton is missing |
| Example 884 | 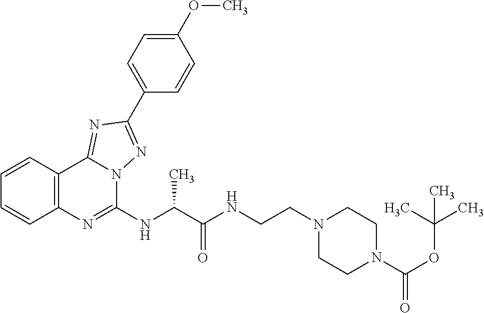 [SMILES: COc1ccc(-c2nc3c4ccccc4nc(N[C@H](C)C(=O)NCCN4CCN(C(=O)OC(C)(C)C)CC4)n3n2)cc1] |
| | tert-butyl 4-[2-({N-[2-(4-methoxyphenyl)[1,2,4]triazolo[1,5-c]quinazolin-5- |
| | yl]-D-alanyl}amino)ethyl]piperazine-1-carboxylate |
| | LC-MS (Method 2): Rt = 1.41 min; MS (ESIpos): m/z = 575 [M + H]⁺ |
| | ¹H-NMR (400 MHz, DMSO-d₆): δ [ppm] = 1.36 (s, 9H), 1.53 (d, 3H), 2.23-2.31 |
| | (m, 4H), 2.34-2.40 (m, 2H), 3.16-3.25 (m, 6H), 3.86 (s, 3H), 4.76 (quin, 1H), |
| | 7.13-7.17 (m, 2H), 7.42-7.46 (m, 1H), 7.61 (d, 1H), 7.72 (ddd, 1H), 7.81 (d, |
| | 1H), 8.10 (t, 1H), 8.23-8.27 (m, 2H), 8.29 (dd, 1H). |
| Example 885 | 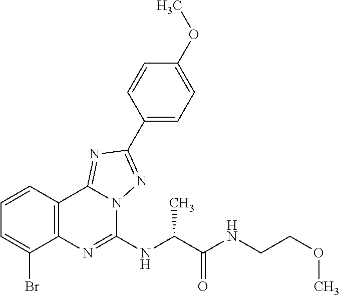 [SMILES: COCCNC(=O)[C@@H](C)Nc1nc2c(Br)cccc2c2nc(-c3ccc(OC)cc3)nn12] |
| | N²-[7-bromo-2-(4-methoxyphenyl)[1,2,4]triazolo[1,5-c]quinazolin-5-yl]-N- |
| | (2-methoxyethyl)-D-alaninamide |
| | LC-MS (Method 2): Rt = 1.37 min; MS (ESIpos): m/z = 499 [M + H]⁺ |
| | ¹H-NMR (400 MHz, DMSO-d₆): δ [ppm] = 1.57 (d, 3H), 3.21 (s, 3H), 3.24-3.40 |
| | (m, 4H and water signal), 3.86 (s, 3H), 4.81 (quin, 1H), 7.13-7.18 (m, 2H), |
| | 7.33 (t, 1H), 8.05 (dd, 1H), 8.10 (d, 1H), 8.23-8.31 (m, 4H). |
| Example 886 | 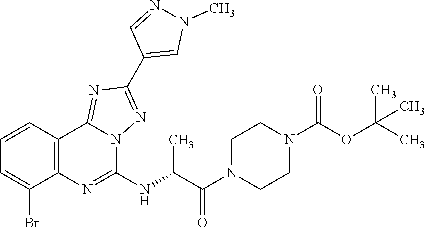 [SMILES: C[C@@H](Nc1nc2c(Br)cccc2c2nc(-c3cnn(C)c3)nn12)C(=O)N1CCN(C(=O)OC(C)(C)C)CC1] |
| | tert-butyl 4-{N-[7-bromo-2-(1-methyl-1H-pyrazol-4-yl)[1,2,4]triazolo[1,5- |
| | c]quinazolin-5-yl]-D-alanyl}piperazine-1-carboxylate |
| | LC-MS (Method 2): Rt = 1.38 min; MS (ESIpos): m/z = 584 [M + H]⁺ |
| | ¹H-NMR (400 MHz, DMSO-d₆): δ [ppm] = 1.42 (s, 9H), 1.53 (d, 3H), 3.40-3.58 |
| | (m, 5H), 3.63-3.77 (m, 2H), 3.96 (s, 3H), 5.20 (quin, 1H), 7.32 (t, 1H), 8.05 |
| | (dd, 1H), 8.07 (s, 1H), 8.12 (br d, 1H), 8.24 (dd, 1H), 8.46 (s, 1H). one proton |
| | is missing |
| Example 887 | 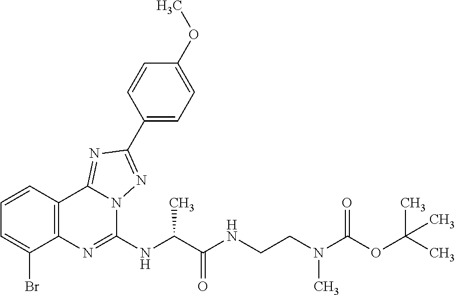 [SMILES: COc1ccc(-c2nc3c4cccc(Br)c4nc(N[C@H](C)C(=O)NCCN(C)C(=O)OC(C)(C)C)n3n2)cc1] |
| | tert-butyl [2-({N-[7-bromo-2-(4-methoxyphenyl)[1,2,4]triazolo[1,5- |
| | c]quinazolin-5-yl]-D-alanyl}amino)ethyl]methylcarbamate |
| | LC-MS (Method 2): Rt = 1.51 min; MS (ESIpos): m/z = 598 [M + H]⁺ |
| | ¹H-NMR (400 MHz, DMSO-d₆): δ [ppm] = 1.34 (s, 9H), 1.58 (d, 3H), 2.76 (br s, |
| | 3H), 3.14-3.31 (m, 4H), 3.86 (s, 3H), 4.73-4.85 (m, 1H), 7.13-7.18 (m, 2H), |
| | 7.33 (t, 1H), 8.05 (dd, 1H), 8.11 (br d, 1H), 8.23-8.33 (m, 4H). |
| Example 888 | 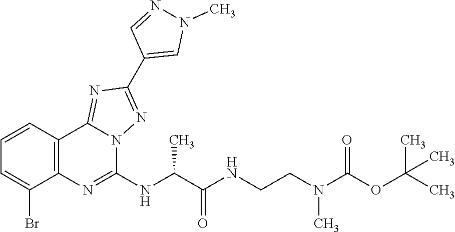 [SMILES: C[C@@H](Nc1nc2c(Br)cccc2c2nc(-c3cnn(C)c3)nn12)C(=O)NCCN(C)C(=O)OC(C)(C)C] |
| | tert-butyl [2-({N-[7-bromo-2-(1-methyl-1H-pyrazol-4-yl)[1,2,4]triazolo[1,5- |
| | c]quinazolin-5-yl]-D-alanyl}amino)ethyl]methylcarbamate |
| | LC-MS (Method 2): Rt = 1.33 min; MS (ESIpos): m/z = 572 [M + H]⁺ |
| | ¹H-NMR (400 MHz, DMSO-d₆): δ [ppm] = 1.33 (s, 9H), 1.56 (br d, 3H), 2.76 (br |
| | d, 3H), 3.15-3.29 (m, 4H), 3.96 (s, 3H), 4.71-4.84 (m, 1H), 7.32 (t, 1H), 7.99 |
| | (br d, 1H), 8.05 (dd, 1H), 8.07 (s, 1H), 8.22-8.35 (m, 2H), 8.46 (s, 1H). |
| Example 889 | 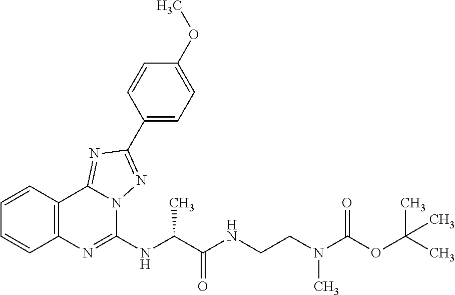 [SMILES: COc1ccc(-c2nc3c4ccccc4nc(N[C@H](C)C(=O)NCCN(C)C(=O)OC(C)(C)C)n3n2)cc1] |
| | tert-butyl [2-({N-[2-(4-methoxyphenyl)[1,2,4]triazolo[1,5-c]quinazolin-5- |
| | yl]-D-alanyl}amino)ethyl]methylcarbamate |
| | LC-MS (Method 2): Rt = 1.35 min; MS (ESIpos): m/z = 520 [M + H]⁺ |
| | ¹H-NMR (400 MHz, DMSO-d₆): δ [ppm] = 1.33 (s, 9H), 1.53 (br d, 3H), 2.73- |
| | 2.82 (m, 3H), 3.14-3.28 (m 4H), 3.86 (s, 3H), 4.69-4.83 (m, 1H), 7.15 (d, |
| | 2H), 7.44 (t, 1H), 7.60 (d, 1H), 7.69-7.74 (m, 1H), 7.78 (br s, 1H), 8.22-8.32 |
| | (m, 4H). |
| Example 890 | 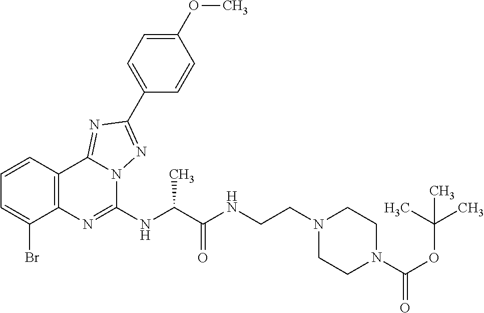 [SMILES: COc1ccc(-c2nc3c4cccc(Br)c4nc(N[C@H](C)C(=O)NCCN4CCN(C(=O)OC(C)(C)C)CC4)n3n2)cc1] |
| | tert-butyl 4-[2-({N-[7-bromo-2-(4-methoxyphenyl)[1,2,4]triazolo[1,5- |
| | c]quinazolin-5-yl]-D-alanyl}amino)ethyl]piperazine-1-carboxylate |
| | LC-MS (Method 2): Rt = 1.47 min; MS (ESIpos): m/z = 653 [M + H]⁺ |
| | ¹H-NMR (400 MHz, DMSO-d₆): δ [ppm] = 1.36 (s, 9H), 1.57 (d, 3H), 2.23-2.31 |
| | (m, 4H), 2.38 (br t, 2H), 3.13-3.26 (m, 6H), 3.86 (s, 3H), 4.79 (quin, 1H), 7.15 |
| | (d, 2H), 7.33 (t, 1H), 8.05 (dd, 1H), 8.09-8.18 (m, 2H), 8.25 (d, 2H), 8.28- |
| | 8.31 (m, 1H). |
| Example 891 | 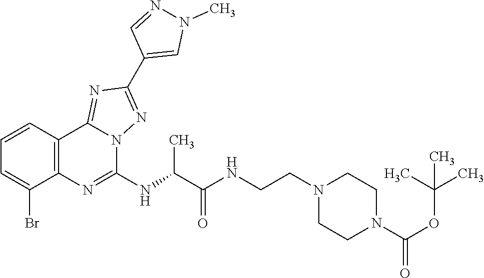 [SMILES: C[C@@H](Nc1nc2c(Br)cccc2c2nc(-c3cnn(C)c3)nn12)C(=O)NCCN1CCN(C(=O)OC(C)(C)C)CC1] |
| | tert-butyl 4-[2-({N-[7-bromo-2-(1-methyl-1H-pyrazol-4- |
| | yl)[1,2,4]triazolo[1,5-c]quinazolin-5-yl]-D-alanyl}amino)ethyl]piperazine- |
| | 1-carboxylate |
| | LC-MS (Method 2): Rt = 1.27 min; MS (ESIpos): m/z = 627 [M + H]⁺ |
| | ¹H-NMR (400 MHz, DMSO-d₆): δ [ppm] = 1.36 (s, 9H), 1.56 (d, 3H), 2.27 (br t, |
| | 4H), 2.37 (br t, 2H), 3.13-3.25 (m, 6H), 3.96 (s, 3H), 4.77 (quin, 1H), 7.32 (t, |
| | 1H), 8.01-8.08 (m, 3H), 8.12 (br t, 1H), 8.24 (dd, 1H), 8.46 (s, 1H). |
| Example 892 | 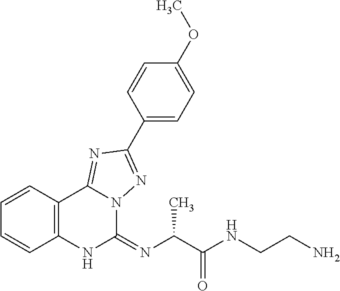 [SMILES: COc1ccc(-c2nc3c4ccccc4[nH]/c(=N/[C@H](C)C(=O)NCCN)n3n2)cc1] |
| | (Z)-N-(2-aminoethyl)-N²-[2-(4-methoxyphenyl)[1,2,4]triazolo[1,5- |
| | c]quinazolin-5(6H)-ylidene]-D-alaninamide |
| | LC-MS (Method 2): Rt = 1.06 min; MS (ESIpos): m/z = 406 [M + H]⁺ |
| | ¹H-NMR (400 MHz, DMSO-d₆): δ [ppm] = 1.53 (d, 3H), 2.59 (t, 2H), 2.99-3.21 |
| | (m, 2H), 3.86 (s, 3H), 4.76 (q, 1H), 7.12-7.18 (m, 2H), 7.41-7.47 (m, 1H), |
| | 7.60-7.65 (m, 1H), 7.72 (ddd, 1H), 8.16 (br t, 1H), 8.23-8.27 (m, 2H), 8.29 |
| | (dd, 1H). two protons are missing |
| Example 893 | 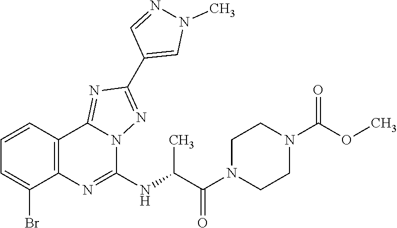 [SMILES: COC(=O)N1CCN(C(=O)[C@@H](C)Nc2nc3c(Br)cccc3c3nc(-c4cnn(C)c4)nn23)CC1] |
| | methyl 4-{N-[7-bromo-2-(1-methyl-1H-pyrazol-4-yl)[1,2,4]triazolo[1,5- |
| | c]quinazolin-5-yl]-D-alanyl}piperazine-1-carboxylate |
| | LC-MS (Method 2): Rt = 1.12 min; MS (ESIpos): m/z = 542 [M + H]⁺ |
| | ¹H-NMR (400 MHz, DMSO-d₆): δ [ppm] = 1.54 (d, 3H), 3.37-3.65 (m, 9H), 3.67- |
| | 3.80 (m, 2H), 3.96 (s, 3H), 5.21 (quin, 1H), 7.32 (t, 1H), 8.04 (dd, 1H), 8.07 |
| | (s, 1H), 8.14 (d, 1H), 8.23 (dd, 1H), 8.45 (s, 1H). |
| Example 894 | 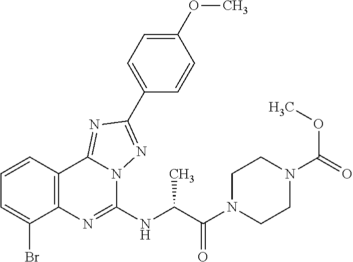 [SMILES: COC(=O)N1CCN(C(=O)[C@@H](C)Nc2nc3c(Br)cccc3c3nc(-c4ccc(OC)cc4)nn23)CC1] |
| | methyl 4-{N-[7-bromo-2-(4-methoxyphenyl)[1,2,4]triazolo[1,5- |
| | c]quinazolin-5-yl]-D-alanyl}piperazine-1-carboxylate |
| | LC-MS (Method 2): Rt = 1.38 min; MS (ESIpos): m/z = 568 [M + H]⁺ |
| | ¹H-NMR (400 MHz, DMSO-d₆): δ [ppm] = 1.55 (d, 3H), 3.37-3.65 (m, 9H), 3.67- |
| | 3.87 (m, 2H), 3.86 (s, 3H), 5.23 (quin, 1H), 7.12-7.17 (m, 2H), 7.33 (t, 1H), |
| | 8.04 (dd, 1H), 8.22-8.33 (m, 4H). |
| Example 895 | 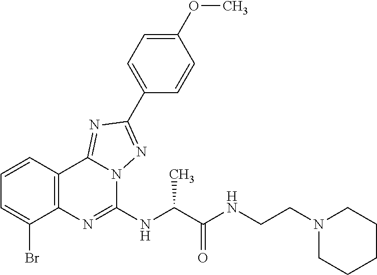 [SMILES: COc1ccc(-c2nc3c4cccc(Br)c4nc(N[C@H](C)C(=O)NCCN4CCCCC4)n3n2)cc1] |
| | N²-[7-bromo-2-(4-methoxyphenyl)[1,2,4]triazolo[1,5-c]quinazolin-5-yl]-N- |
| | [2-(piperidin-1-yl)ethyl]-D-alaninamide |
| | LC-MS (Method 2): Rt = 1.47 min; MS (ESIpos): m/z = 552 [M + H]⁺ |
| | ¹H-NMR (400 MHz, DMSO-d₆): δ [ppm] = 1.22-1.38 (m, 6H), 1.57 (d, 3H), 2.19- |
| | 2.35 (m, 6H), 3.17-3.24 (m, 2H), 3.86 (s, 3H), 4.74-4.83 (m, 1H), 7.12- |
| | 7.17 (m, 2H), 7.33 (t, 1H), 8.03-8.09 (m, 2H), 8.15 (br d, 1H), 8.22-8.27 (m, |
| | 2H), 8.29 (dd, 1H). |
| Example 896 | 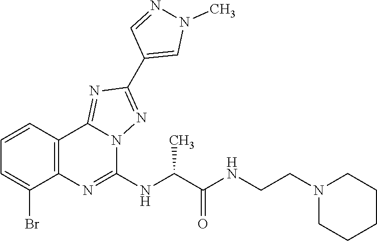 [SMILES: C[C@@H](Nc1nc2c(Br)cccc2c2nc(-c3cnn(C)c3)nn12)C(=O)NCCN1CCCCC1] |
| | N²-[7-bromo-2-(1-methyl-1H-pyrazol-4-yl)[1,2,4]triazolo[1,5-c]quinazolin- |
| | 5-yl]-N-[2-(piperidin-1-yl)ethyl]-D-alaninamide |
| | LC-MS (Method 2): Rt = 1.21 min; MS (ESIpos): m/z = 526 [M + H]⁺ |
| | ¹H-NMR (400 MHz, DMSO-d₆): δ [ppm] = 1.22-1.38 (m, 6H), 1.55 (d, 3H), 2.20- |
| | 2.35 (m, 6H), 3.16-3.24 (m, 2H), 3.96 (s, 3H), 4.77 (q, 1H), 7.32 (t, 1H), 7.81- |
| | 8.09 (m, 4H), 8.24 (dd, 1H), 84.6 (s, 1H). |
| Example 897 | 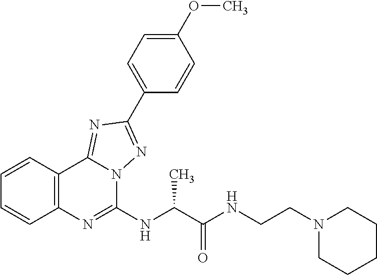 [SMILES: COc1ccc(-c2nc3c4ccccc4nc(N[C@H](C)C(=O)NCCN4CCCCC4)n3n2)cc1] |
| | N²-[2-(4-methoxyphenyl)[1,2,4]triazolo[1,5-c]quinazolin-5-yl]-N-[2- |
| | (piperidin-1-yl)ethyl]-D-alaninamide |
| | LC-MS (Method 2): Rt = 1.35 min; MS (ESIpos): m/z = 474 [M + H]⁺ |
| | ¹H-NMR (400 MHz, DMSO-d₆): δ [ppm] = 1.24-1.41 (m, 6H), 1.52 (d, 3H), 2.20- |
| | 2.35 (m, 6H), 3.19 (q, 2H), 3.86 (s, 3H), 4.76 (quin, 1H), 7.12-7.18 (m, 2H), |
| | 7.44 (ddd, 1H), 7.62 (d, 1H), 7.72 (ddd, 1H), 7.81 (d, 1H), 8.05 (t, 1H), 8.23- |
| | 8.31 (m, 3H). |
| Example 898 | 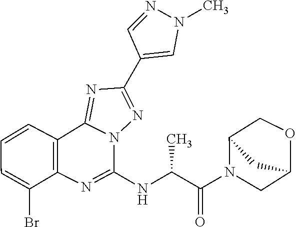 [SMILES: C[C@@H](Nc1nc2c(Br)cccc2c2nc(-c3cnn(C)c3)nn12)C(=O)N1C[C@H]2C[C@@H]1CO2] |
| | (2R)-2-{[7-bromo-2-(1-methyl-1H-pyrazol-4-yl)[1,2,4]triazolo[1,5- |
| | c]quinazolin-5-yl]amino}-1-[(1R,4R)-2-oxa-5-azabicyclo[2.2.1]heptan-5- |
| | yl]propan-1-one |
| | LC-MS (Method 2): Rt = 1.04 min; MS (ESIpos): m/z = 497 [M + H]⁺ |
| | Rotational isomers A:B = 10:6 |
| | ¹H-NMR (400 MHz, DMSO-d₆): δ [ppm] = 1.53 (d, 3H, A), 1.59 (d, 3H, B), 1.80 |
| | (s, 2H, A), 1.85-1.95 (m, 2H, B), 3.26 (br s, 1H), 3.53 (br d, 1H), 3.64 (d, 1H, |
| | A), 3.69-3.78 (m, 1H, A; 1H, B), 3.90-4.03 (m, 4H, A; 4H, B, A), 4.62 (s, 1H, |
| | B), 4.74-4.84 (m, 3H, A), 4.88 (s, 1H, B), 5.11-5.20 (m, 1H, B), 7.29-7.36 |
| | (m, 1H, A; 1H, B), 7.99 (br s, 1H, B), 8.02-8.09 (m, 2H, A; 2H, B), 8.19 (br s, |
| | 1H, A), 8.21-8.26 (m, 1H, A; 1H, B), 8.45-8.46 (m, 1H, A; 1H, B). |
| Example 899 | 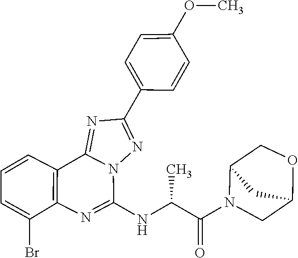 [SMILES: COc1ccc(-c2nc3c4cccc(Br)c4nc(N[C@H](C)C(=O)N4C[C@H]5C[C@@H]4CO5)n3n2)cc1] |
| | (2R)-2-{[7-bromo-2-(4-methoxyphenyl)[1,2,4]triazolo[1,5-c]quinazolin-5- |
| | yl]amino}-1-[(1R,4R)-2-oxa-5-azabicyclo[2.2.1]heptan-5-yl]propan-1-one |
| | LC-MS (Method 2): Rt = 1.26 min; MS (ESIpos): m/z = 523 [M + H]⁺ |
| | Rotational isomers A:B = 10:6 |
| | ¹H-NMR (400 MHz, DMSO-d₆): δ [ppm] = 1.54 (d, 3H, A), 1.60 (d, 3H, B), 1.81 |
| | (s, 2H, A), 1.85-1.96 (m, 2H, B), 3.54 (d, 1H, A), 3.64 (d, 1H, A), 3.70-3.77 |
| | (m, 1H, A; 1H, B), 3.86 (s, 3H, A; 3H, B), 3.98-4.05 (m, 1H, A; 1H, B), 4.60- |
| | 4.64 (m, 1H, B), 4.77-4.88 (m, 3H, A), 4.89 (s, 1H, B), 5.14-5.23 (m, 1H, B), |
| | 7.12-7.18 (m, 2H, A; 2H, B), 7.33 (t, 1H, A; 1H, B), 8.01-8.07 (m, 1H, A; 1H, |
| | B), 8.18-8.31 (m, 3H, A; 4H, B), 8.36 (br d, 1H, A). |
| Example 900 | 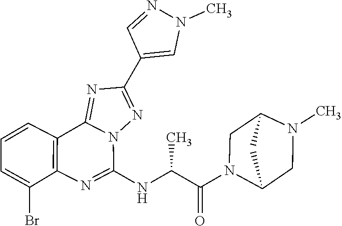 [SMILES: C[C@@H](Nc1nc2c(Br)cccc2c2nc(-c3cnn(C)c3)nn12)C(=O)N1C[C@@H]2C[C@H]1CN2C] |
| | (2R)-2-{[7-bromo-2-(1-methyl-1H-pyrazol-4-yl[1,2,4]triazolo[1,5- |
| | c]quinazolin-5-yl]amino}-1-[(1S,4S)-5-methyl-2,5- |
| | diazabicyclo[2.2.1]heptan-2-yl]propan-1-one |
| | LC-MS (Method 2): Rt = 1.05 min; MS (ESIpos): m/z = 510 [M + H]⁺ |
| | Rotational isomers A:B = 1:1 |
| | ¹H-NMR (400 MHz, DMSO-d₆): δ [ppm] = 1.51 (d, 3H), 1.53 (d, 3H), 1.65 (br d, |
| | 1H), 7.171 (br d, 1H), 1.79 (br d, 1H), 1.90 (br d, 1H), 2.31 (s, 3H), 2.32 (s, 3H), |
| | 2.58 (br d, 2H), 2.90 (dd, 1H), 3.19 (dd, 1H), 3.39 (s, 1H), 3.47-3.53 (m, 2H), |
| | 3.93-3.98 (m, 7H), 4.51 (s, 1H), 4.85 (s, 1H), 4.97 (quin, 1H), 5.18 (quin, 1H), |
| | 7.32 (t, 1H), 7.32 (t, 1H), 7.95 (d, 1H), 8.01-8.09 (m, 5H), 8.21-8.25 (m, 2H), |
| | 8.46 (s, 1H), 8.46 (s, 1H). |
| Example 901 | 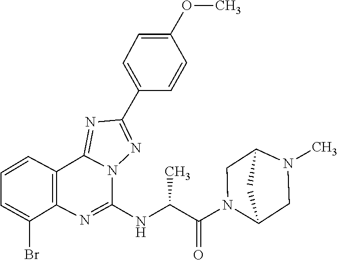 [SMILES: COc1ccc(-c2nc3c4cccc(Br)c4nc(N[C@H](C)C(=O)N4C[C@@H]5C[C@H]4CN5C)n3n2)cc1] |
| | (2R)-2-{[7-bromo-2-(4-methoxyphenyl)[1,2,4]triazolo[1,5-c]quinazolin-5- |
| | yl]amino}-1-[(1S,4S)-5-methyl-2,5-diazabicyclo[2.2.1]heptan-2-yl]propan- |
| | 1-one |
| | LC-MS (Method 2): Rt = 1.33 min; MS (ESIpos): m/z = 536 [M + H]⁺ |
| | Rotational isomer A:B = 1:1 |
| | ¹H-NMR (400 MHz, DMSO-d₆): δ [ppm] = 1.53 (d, 3H), 1.55 (d, 3H), 1.66 (br d, |
| | 1H), 1.73 (br d, 1H), 1.80 (br d, 1H), 1.91 (br d, 1H), 2.33 (s, 3H), 2.33 (s, 3H), |
| | 2.57-2.63 (m, 1H), 2.65-2.69 (m, 2H), 2.90 (dd, 1H), 3.20 (dd, 1H), 3.40- |
| | 3.35 (m, 2H), 3.48-3.54 (m, 2H), 3.86 (s, 6H), 4.00 (d, 1H), 4.53 (s, 1H), 4.90 |
| | (s, 1H), 5.00 (quin, 1H), 5.16-5.25 (m, 1H), 7.09-7.16 (m, 4H), 7.31 (t, 1H), |
| | 7.32 (t, 1H), 8.00-8.06 (m, 2H), 8.16-8.30 (m, 8H). |
| Example 902 | 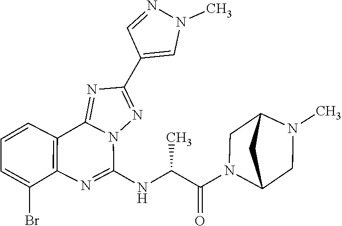 [SMILES: C[C@@H](Nc1nc2c(Br)cccc2c2nc(-c3cnn(C)c3)nn12)C(=O)N1C[C@H]2C[C@@H]1CN2C] |
| | (2R)-2-{[7-bromo-2-(1-methyl-1H-pyrazol-4-yl[1,2,4]triazolo[1,5- |
| | c]quinazolin-5-yl]amino}-1-[(1R,4R)-5-methyl-2,5- |
| | diazabicyclo[2.2.1]heptan-2-yl]propan-1-one |
| | LC-MS (Method 2): Rt = 0.99 min; MS (ESIpos): m/z = 510 [M + H]⁺ |
| | Rotational isomers A:B = 1:2 |
| | ¹H-NMR (400 MHz, DMSO-d₆): δ [ppm] = 1.52 (d, 3H, A), 1.56 (d, 3H, B), 1.63 |
| | (br d, 1H, A), 1.73 (br d, 1H, B), 1.80 (br d, 1H, A), 1.88 (br d, 1H, B), 2.26 (s, |
| | 3H, B), 2.34 (s, 3H, A), 2.37 (d, 1H, A), 2.74-2.82 (m, 2H, A), 3.14 (dd, 1H, |
| | B), 3.43 (br d, 1H, B), 3.56 (s, 1H), A), 3.67 (d, 1H, A), 3.81 (d, 1H, B), 3.83 (d, |
| | 1H, B), 3.96 (s, 3H, A; 3H, B), 4.56 (s, 1H, A), 4.60 (s, 1H, B), 4.82 (quin, 1H, |
| | A), 5.10 (quin, 1H, B), 7.33 (t, 1H, A), 7.33 (t, 1H, B), 7.90 (br d, 1H, B), 8.01- |
| | 8.09 (m, 2H, A; 2H, B), 8.13 (br d, 1H, A), 8.23 (dd, 1H, A), 8.24 (dd, 1H, B), |
| | 8.44 (s, 1H, A), 8.46 (s, 1H, B). |
| Example 903 | 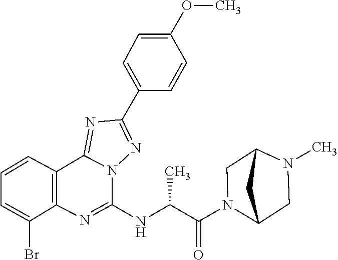 [SMILES: COc1ccc(-c2nc3c4cccc(Br)c4nc(N[C@H](C)C(=O)N4C[C@H]5C[C@@H]4CN5C)n3n2)cc1] |
| | (2R)-2-{[7-bromo-2-(4-methoxyphenyl)[1,2,4]triazolo[1,5-c]quinazolin-5- |
| | yl]amino}-1-[(1R,4R)-5-methyl-2,5-diazabicyclo[2.2.1]heptan-2- |
| | yl]propan-1-one |
| | LC-MS (Method 2): Rt = 1.25 min; MS (ESIpos): m/z = 536 [M + H]⁺ |
| | Rotational isomers A:B = 2:1 |
| | ¹H-NMR (400 MHz, DMSO-d₆): δ [ppm] = 1.54 (d, 3H, A), 1.58 (d, 3H, B), 1.65 |
| | (br d, 1H, A), 1.73 (br d, 1H, B), 1.80 (br d, 1H, A), 1.88 (br d, 1H, B), 2.27 (s, |
| | 3H, B), 2.35 (s, 3H, A), 2.38 (d, 1H, A), 2.76-2.83 (m, 2H, A), 3.15 (dd, 1H, |
| | B), 3.39 (br s, 1H, B), 3.44 (br d, 1H, B), 3.56 (s, 1H, A), 3.68 (d, 1H, A), 3.82- |
| | 3.87 (m, 3H, A; 5H, B), 4.57 (s, 1H, A), 4.62 (s, 1H, B), 4.84 (quin, 1H, A), |
| | 5.14 (quin, 1H, B), 7.11-7.16 (m, 2H, A; 2H, B), 7.31 (t, 1H, A), 7.32 (t, 1H, |
| | B), 8.01-8.06 (m, 1H, A, 1H, B), 8.14 (br d, 1H, B), 8.21-8.30 (m, 3H), A; 3H, |
| | B), 8.32 br d, 1H, A). |
| Example 904 | 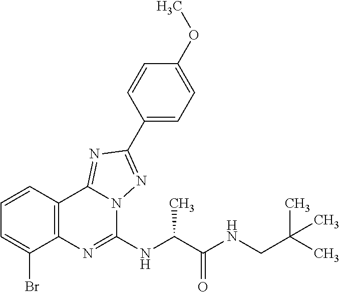 [SMILES: COc1ccc(-c2nc3c4cccc(Br)c4nc(N[C@H](C)C(=O)NCC(C)(C)C)n3n2)cc1] |
| | N²-[7-bromo-2-(4-methoxyphenyl)[1,2,4]triazolo[1,5-c]quinazolin-5-yl]-N- |
| | (2-hydroxy-2-methylpropyl)-D-alaninamide |
| | LC-MS (Method 2): Rt = 1.21 min; MS (ESIpos): m/z = 513 [M + H]⁺ |
| | ¹H-NMR (400 MHz, DMSO-d₆): δ [ppm] = 1.04 (s, 3H), 1.06 (s, 3H), 1.60 (d, |
| | 3H), 3.06 (dd, 1H), 3.14 (dd, 1H), 3.86 (s, 3H), 4.44 (s, 1H), 4.89 (qun, 1H), |
| | 7.12-7.18 (m, 2H), 7.33 (t, 1H), 8.03-8.13 (m, 3H), 8.22-8.27 (m, 2H), 8.29 |
| | (dd, 1H). |
| Example 905 | 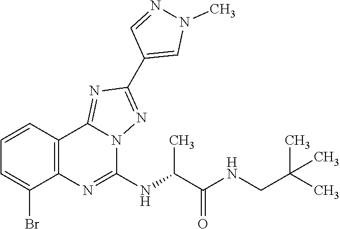 [SMILES: C[C@@H](Nc1nc2c(Br)cccc2c2nc(-c3cnn(C)c3)nn12)C(=O)NCC(C)(C)C] |
| | N²-[7-bromo-2-(1-methyl-1H-pyrazol-4-yl)[1,2,4]triazolo[1,5-c]quinazolin- |
| | 5-yl]-N-(2-hydroxy-2-methylpropyl)-D-alaninamide |
| | LC-MS (Method 2): Rt = 0.94 min; MS (ESIpos): m/z = 487 [M + H]⁺ |
| | ¹H-NMR (400 MHz, DMSO-d₆): δ [ppm] = 1.03 (s, 3H), 1.06 (s, 3H), 1.58 (d, |
| | 3H), 3.06 (dd, 1H), 3.13 (dd, 1H), 3.96 (s, 3H), 4.44 (s, 1H), 4.88 (quin, 1H), |
| | 7.32 (t, 1H), 7.95 (d, 1H), 8.05 (dd, 1H), 8.07 (s, 1H), 8.11 (br t, 1H), 8.24 (dd, |
| | 1H), 8.47 (s, 1H). |
| Example 906 | 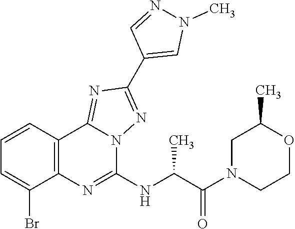 [SMILES: C[C@@H]1CN(C(=O)[C@@H](C)Nc2nc3c(Br)cccc3c3nc(-c4cnn(C)c4)nn23)CCO1] |
| | (2R)-2-{[7-bromo-2-(1-methyl-1H-pyrazol-4-yl)[1,2,4]triazolo[1,5- |
| | c]quinazolin-5-yl]amino}-1-[(2R)-2-methylmorpholin-4-yl]propan-1-one |
| | LC-MS (Method 2): Rt = 1.10 min; MS (ESIpos): m/z = 499 [M + H]⁺ |
| | Rotational isomers A:B = 1:1 |
| | ¹H-NMR (400 MHz, DMSO-d₆): δ [ppm] = 1.09 (b r d, 3H), 1.18 (br d, 3H), 1.53 |
| | (br t, 6H), 2.69-2.79 (m, 1H), 3.03-3.12 (m, 1H), 3.38-3.47 (m, 2H), 3.50- |
| | 3.66 (m, 2H), 3.82-3.93 (m, 2H), 3.93-4.06 (m, 8H), 4.17 (br d, 1H), 4.24 (br |
| | d, 1H), 5.11-5.19 (m, 1H), 5.21-5.30 (m, 1H), 7.28-7.36 (m, 2H), 8.00-8.08 |
| | (m, 5H), 8.17-8.27 (m, 3H), 8.45 (s, 2H). |
| Example 907 | 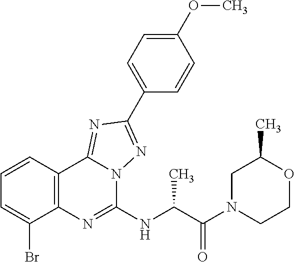 [SMILES: COc1ccc(-c2nc3c4cccc(Br)c4nc(N[C@H](C)C(=O)N4CCO[C@H](C)C4)n3n2)cc1] |
| | (2R)-2-{[7-bromo-2-(4-methoxyphenyl)[1,2,4]triazolo[1,5-c]quinazolin-5- |
| | yl]amino}-1-[(2R)-2-methylmorpholin-4-yl]propan-1-one |
| | LC-MS (Method 2): Rt = 1.38 min; MS (ESIpos): m/z = 525 [M + H]⁺ |
| | Rotational Isomers A:B = 10:8 |
| | ¹H-NMR (400 MHz, CHLOROFORM-d): δ [ppm] = 1.20 (d, 3H, A), 1.31 (d, 2H, |
| | B), 1.64 (d, 3H, A), 1.67 (d, 3H, B), 2.56 (dd, 1H, A), 2.86-2.95 (m, 1H, B), |
| | 3.16 (dd, 1H, B), 3.37-3.46 (m, 1H, A), 3.49-3.73 (m, 2H, A; 2H, B), 3.90 (s, |
| | 3H, A; 3H, B), 3.93-4.03 (m, 1H, A, 2H, B), 4.07-4.13 (m, 1H, A), 4.41 (br d, |
| | 1H, B), 4.49 (br d, 1H, A), 5.36-5.43 (m, 1H, B), 5.44-5.52 (m, 1H, A), 7.01- |
| | 7.06 (m, 2H, A; 2H, B), 7.12 (br d, 1H, B), 7.19 (br d, 1H, A), 7.22-7.28 (m, |
| | 1H, A; 1H, B, and chloroform signal), 7.95 (dd, 1H, A; 1H, B), 8.26-8.32 (m, |
| | 2H, A; 2H, B), 8.38 (dd, 1H, A; 1H, B). |
| Example 908 | 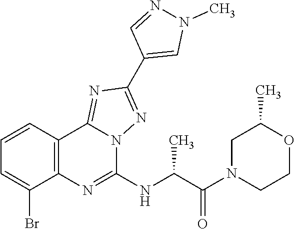 [SMILES: C[C@H]1CN(C(=O)[C@@H](C)Nc2nc3c(Br)cccc3c3nc(-c4cnn(C)c4)nn23)CCO1] |
| | (2R)-2-{[7-bromo-2-(1-methyl-1H-pyrazol-4-yl)[1,2,4]triazolo[1,5- |
| | c]quinazolin-5-yl]amino}-1-[(2S)-2-methylmorpholin-4-yl]propan-1-one |
| | LC-MS (Method 2): Rt = 1.10 min; MS (ESIpos): m/z = 499 [M + H]⁺ |
| | Rotational isomers A:B = 10:8 |
| | ¹H-NMR (400 MHz, METHANOL-d₄): δ [ppm] = 1.19-1.23 (m, 3H, A; 3H, B), |
| | 1.60 (d, 3H, B), 1.64 (d, 3H, A), 2.59 (dd, 1H, A), 2.85-2.95 (m, 1H, B), 3.08 |
| | (dd, 1H, B), 3.49-3.69 (m, 3H, A; 1H, B), 3.76-3.85 (m, 1H, B), 3.93 (br dd, |
| | 1H, B), 3.97 (s, 3H, A; 3H, B), 3.99-4.07 (m, 2H, A), 4.17 (br d, 1H, B), 4.33- |
| | 4.41 (m, 1H, A; 1H, B), 5.23 (q, 1H, A), 5.46 (q, 1H, B), 7.23-7.28 (m, 1H, |
| | A, 1H, B), 7.92-7.97 (m, 1H, A; 1H, B), 8.04 (s, 1H, A; 1H, B), 8.17 (s, 1H, A; |
| | 1H, B), 8.20-8.25 (m, 1H, A; 1H, B). |
| Example 909 | 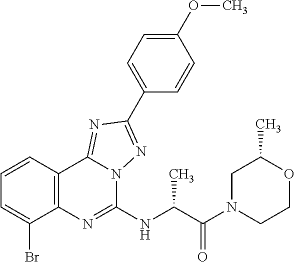 [SMILES: COc1ccc(-c2nc3c4cccc(Br)c4nc(N[C@H](C)C(=O)N4CCO[C@@H](C)C4)n3n2)cc1] |
| | (2R)-2-{[7-bromo-2-(4-methoxyphenyl)[1,2,4]triazolo[1,5-c]quinazolin-5- |
| | yl]amino}-1-[(2S)-2-methylmorpholin-4-yl]propan-1-one |
| | LC-MS (Method 2): Rt = 1.38 min; MS (ESIpos): m/z = 525 [M + H]⁺ |
| | Rotational isomers A:B = 1:1 |
| | ¹H-NMR (400 MHz, DMSO-d₆): δ [ppm] = 1.08-1.16 (m, 6H), 1.52-1.54 (m, |
| | 6H), 2.40-2.44 (m, 1H), 2.74-2.83 (m, 1H), 2.96-3.03 (m, 1H), 3.41-3.57 |
| | (m, 2H), 3.62-3.71 (m, 1H), 3.81-3.88 (m, 7H), 3.91-4.01 (m, 2H), 4.03- |
| | 4.11 (m, 1H), 4.16-4.26 (m, 2H), 5.11-5.20 (m, 1H), 5.28-5.37 (m 1H), 7.13- |
| | 7.18 (m, 4H), 7.30-7.36 (m, 2H), 8.03-8.08 (m, 2H), 8.21-8.32 (m, 7H), |
| | 8.34 (br d, 1H). |
| Example 910 | 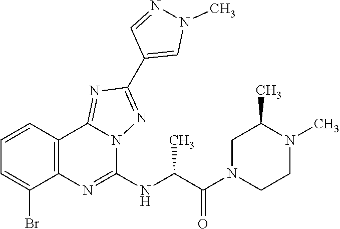 [SMILES: C[C@@H]1CN(C(=O)[C@@H](C)Nc2nc3c(Br)cccc3c3nc(-c4cnn(C)c4)nn23)CCN1C] |
| | (2R)-2-{[7-bromo-2-(1-methyl-1H-pyrazol-4-yl)[1,2,4]triazolo[1,5- |
| | c]quinazolin-5-yl]amino}-1-[(3R)-3,4-dimethylpiperazin-1-yl]propan-1- |
| | one |
| | LC-MS (Method 2): Rt = 1.11 min; MS (ESIpos): m/z = 512 [M + H]⁺ |
| | Rotational Isomers A:B = 1:1 |
| | ¹H-NMR (400 MHz, DMSO-d₆): δ [ppm] = 0.97 (br d, 3H), 1.08 (br d, 3H), 1.52 |
| | (d, 6H), 1.93-2.07 (m, 3H), 2.18 (s, 3H), 2.20 (s, 3H), 2.25-2.31 (m, 1H), |
| | 2.72-2.84 (m, 3H), 2.98-3.07 (m, 1H), 3.90-4.01 (m, 8H), 4.08 (br d, 1H), |
| | 4.19 (br d, 1H), 5.15-5.29 (m, 2H), 7.28-7.36 (m, 1H), 7.95-8.10 (m, 5H), |
| | 8.16 (br s, 1H), 8.24 (br d, 2H), 8.45 (s, 2H). |
| Example 911 | 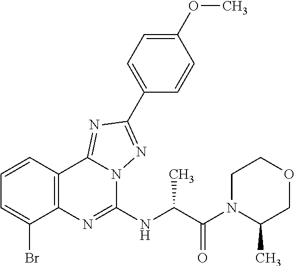 [SMILES: COc1ccc(-c2nc3c4cccc(Br)c4nc(N[C@H](C)C(=O)N4CCOC[C@H]4C)n3n2)cc1] |
| | (2R)-2-{[7-bromo-2-(4-methoxyphenyl)[1,2,4]triazolo[1,5-c]quinazolin-5- |
| | yl]amino}-1-[(3R)-3-methylmorpholin-4-yl]propan-1-one |
| | LC-MS (Method 2): Rt = 1.42 min; MS (ESIpos): m/z = 525 [M + H]⁺ |
| | Rotational isomers A:B = 3:1 |
| | ¹H-NMR (400 MHz, DMSO-d₆): δ [ppm] = 1.16 (br d, 3H, A), 1.42 (br s, 3H, B), |
| | 1.56 (br d, 3H, A; 3H, B), 2.98-3.12 (m, 1H, B), 3.41-3.53 (m, 2H, A), 3.60- |
| | 3.96 (m, 7H, A; 7H, B), 4.08-4.18 (m, 1H, B), 4.34 (br d, 1H, A), 4.42-4.53 |
| | (m, 1H, B), 5.06-5.16 (m, 1H, A), 5.33-5.45 (m, 1H, B), 7.12-7.17 (m, 2H, |
| | A; 2H, B), 7.32 (t, 1H, A; 1H, B), 8.04 (br d, 1H, A; 1H, B), 8.23 -8.33 (m, 4H, |
| | A; 4H), B). |
| Example 912 | 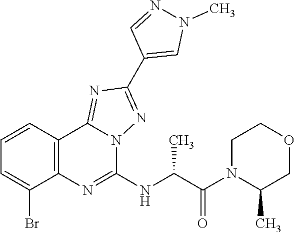 [SMILES: C[C@@H]1COCCN1C(=O)[C@@H](C)Nc1nc2c(Br)cccc2c2nc(-c3cnn(C)c3)nn12] |
| | (2R)-2-{[7-bromo-2-(1-methyl-1H-pyrazol-4-yl)[1,2,4]triazolo[1,5- |
| | c]quinazolin-5-yl]amino}-1-[(3R)-3-methylmorpholin-4-yl]propan-1-one |
| | LC-MS (Method 2): Rt = 1.12 min; MS (ESIpos): m/z = 499 [M + H]⁺ |
| | Rotational isomers A:B = 2:1 |
| | ¹H-NMR (400 MHz, DMSO-d₆): δ [ppm] = 1.15 (br d, 3H, A), 1.42 (br s, 3H, B), |
| | 1.55 (br d, 3H, A; 3H, B), 2.97-3.10 (m, 1H, B), 3.43-3.53 (m, 1H, A; 1H, B), |
| | 3.60-3.99 (m, 7H, A; 5H, B), 4.07-4.17 (m, 1H, B), 4.29-4.39 (m, 1H, A), |
| | 4.44 (br s, 1H, B), 5.03-5.13 (m, 1H, A), 5.37 (br s, 1H, B), 7.32 (t, 1H, A; 1H, |
| | B), 8.04 (br d, 1H, A; 2H, B), 8.07 (s, 1H, A; 1H, B), 8.15 (br d, 1H, A), 8.23 (d, |
| | 1H, A; 1H, B), 8.45 (s, 1H, A; 1H, B). |
| Example 913 | 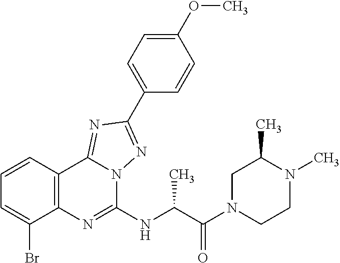 [SMILES: COc1ccc(-c2nc3c4cccc(Br)c4nc(N[C@H](C)C(=O)N4CCN(C)[C@H](C)C4)n3n2)cc1] |
| | (2R)-2-{[7-bromo-2-(4-methoxyphenyl)[1,2,4]triazolo[1,5-c]quinazolin-5- |
| | yl]amino}-1-[(3R)-3,4-dimethylpiperazin-1-yl]propan-1-one |
| | LC-MS (Method 2): Rt = 1.41 min; MS (ESIpos): m/z = 538 [M + H]⁺ |
| | Rotational isomers A:B = 1:1 |
| | ¹H-NMR (400 MHz, DMSO-d₆): δ [ppm] = 0.97 (d, 3H), 1.08 (d, 3H), 1.54 (d, |
| | 6H), 1.93-2.08 (m, 3H), 2.19 (s, 3H), 2.20 (s, 3H), 2.27-2.36 (m, 1H), 2.73- |
| | 2.85 (m, 3H), 3.00-3.08 (m, 1H), 3.36-3.42 (m, 2H), 3.86 (s, 6H), 3.98 (br t, |
| | 2H), 4.09 (br d, 1H), 4.21 (br d, 1H), 5.17-5.32 (m, 2H), 7.15 (d, 4H), 7.33 (t, |
| | 2H), 8.05 (dd, 2H), 8.19 (br d, 1H), 8.25 (d, 4H), 8.29 (d, 2H), 8.32 (br d, 1H). |
| Example 914 | 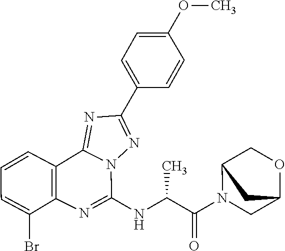 [SMILES: COc1ccc(-c2nc3c4cccc(Br)c4nc(N[C@H](C)C(=O)N4C[C@@H]5C[C@H]4CO5)n3n2)cc1] |
| | (2R)-2-{[7-bromo-2-(4-methoxyphenyl)[1,2,4]triazolo[1,5-c]quinazolin-5- |
| | yl]amino}-1-[(1S,4S)-2-oxa-5-azabicyclo[2.2.1]heptan-5-yl]propan-1-one |
| | LC-MS (Method 2): Rt = 1.30 min; MS (ESIpos): m/z = 523 [M + H]⁺ |
| | Rotational isomers A:B = 3:2 |
| | ¹H-NMR (400 MHz, CHLOROFORM-d): δ [ppm] = 1.62 (d, 3H, A), 1.68 (d, 3H, |
| | B), 1.89 (dd, 1H, A), 1.93-2.05 (m, 1H, A; 2H, B), 3.51 (s, 2H, B), 3.63 (dd, |
| | 1H, A), 3.68-3.76 (m, 1H), 3.82 (dd, 1H, A), 3.90 (s, 3H, A; 3H, B), 3.96 (d, |
| | 1H, B), 3.99-4.03 (m, 1H, A; 1H, B), 4.16 (d, 1H, A), 4.70 (d, 1H, B), 4.76 (s, |
| | 1H, A), 4.96 (s, 1H, A), 5.15 (quin, 1H, A), 5.23 (s, 1H, B), 5.37-5.46 (m, 1H, |
| | B), 6.98 (d, 1H, A), 7.05-7.05 (m, 2H, A; 2H, B), 7.12 (d, 1H, B), 7.21-7.28 |
| | (m, 1H, A; 1H, B, and chloroform signal), 7.92-7.97 (m, 1H, A; 1H, B), 8.25- |
| | 8.30 (m, 2H, A; 2H, B), 8.37 (dt, 1H, A; 1H, B). |
| Example 915 | 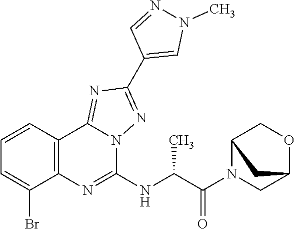 [SMILES: C[C@@H](Nc1nc2c(Br)cccc2c2nc(-c3cnn(C)c3)nn12)C(=O)N1C[C@@H]2C[C@H]1CO2] |
| | (2R)-2-{[7-bromo-2-(1-methyl-1H-pyrazol-4-yl)[1,2,4]triaozlo[1,5- |
| | c]quinazolin-5-yl]amino}-1-[(1S,4S)-2-oxa-5-azabicyclo[2.2.1]heptna-5- |
| | yl]propan-1-one |
| | LC-MS (Method 2): Rt = 1.01 min; MS (ESIpos): m/z = 497 [M + H]⁺ |
| | Rotational isomers A:B = 3:2 |
| | ¹H-NMR (400 MHz, CHLOROFORM-d): δ [ppm] = 1.61 (d, 3H, A), 1.67 (d, 3H, |
| | B), 1.89 (dd, 1H, A), 1.92-2.05 (m, 1H, A; 2H, B), 3.51 (s, 2H, B), 3.62 (dd, |
| | 1H, A), 3.81 (dd, 1H, A), 3.96 (d, 1H, B), 3.98-4.02 (m, 4H, A; 4H, B), 4.14 (d, |
| | 1H, A), 4.69 (d, 1H, B), 4.76 (s, 1H, A), 4.95 (s, 1H, A), 5.13 (quin, 1H, A), 5.20 |
| | (s, 1H, B), 5.35-5.44 (m, 1H, B), 6.90 (d, 1H, A), 7.06 (d, 1H, B), 7.21-7.27 |
| | (m, 1H, A; 1H B, and chloroform signal), 7.93-7.97 (m, 1H, A; 1H, B), 8.09 |
| | (s, 1H, A; 1H, B), 8.17 (d, 1H, A; 1H, B), 8.30-8.35 (m, 1H, A; 1H, B). |
| Example 916 | 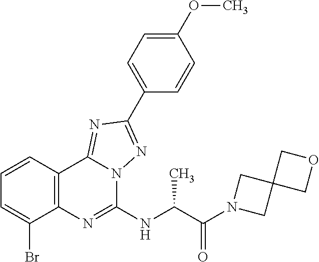 [SMILES: COc1ccc(-c2nc3c4cccc(Br)c4nc(N[C@H](C)C(=O)N4CC5(COC5)C4)n3n2)cc1] |
| | (2R)-2-{[7-bromo-2-(4-methoxyphenyl)[1,2,4]triazolo[1,5-c]quinazolin-5- |
| | yl]amino}-1-(2-oxa-6-azaspiro[3.3]heptan-6-yl)propan-1-one |
| | LC-MS (Method 2): Rt = 1.25 min; MS (ESIpos): m/z = 523 [M + H]⁺ |
| | ¹H-NMR (400 MHz, DMSO-d₆): δ [ppm] = 1.51 (d, 3H), 3.86 (s, 3H), 4.08-4.16 |
| | (m, 2H), 4.50 (d, 1H), 4.65-4.83 (m, 6H), 7.12-7.17 (m, 2H), 7.33 (t, 1H), |
| | 8.05 (dd, 1H), 8.22-8.26 (m, 2H), 8.27-8.34 (m, 2H). |
| Example 917 | 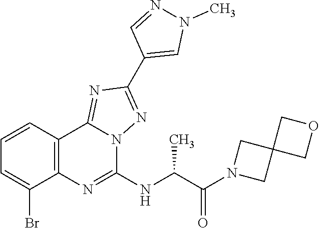 [SMILES: C[C@@H](Nc1nc2c(Br)cccc2c2nc(-c3cnn(C)c3)nn12)C(=O)N1CC2(COC2)C1] |
| | (2R)-2-{[7-bromo-2-(1-methyl-1H-pyrazol-4-yl)[1,2,4]triazolo[1,5- |
| | c]quinazolin-5-yl]amino}-1-(2-oxa-6-azaspiro[3.3]heptan-6-yl)propan-1- |
| | one |
| | LC-MS (Method 2): Rt = 0.97 min; MS (ESIpos): m/z = 497 [M + H]⁺ |
| | ¹H-NMR (400 MHz, DMSO-d₆): δ [ppm] = 1.49 (d, 3H), 3.96 (s, 3H), 4.06-4.15 |
| | (m, 2H), 4.49 (d, 1H), 4.65-4.81 (m, 6H), 7.33 (t, 1H), 8.03-8.08 (m, 2H), |
| | 8.15 (d, 1H), 8.24 (dd, 1H), 8.44 (s, 1H). |
| Example 918 | 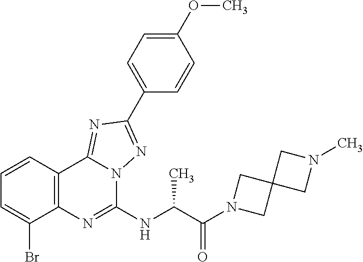 [SMILES: COc1ccc(-c2nc3c4cccc(Br)c4nc(N[C@H](C)C(=O)N4CC5(CN(C)C5)C4)n3n2)cc1] |
| | (2R)-2-{[7-bromo-2-(4-methoxyphenyl)[1,2,4]triazolo[1,5-c]quinazolin-5- |
| | yl]amino}-1-(6-methyl-2,6-diazaspiro[3.3]heptan-2-yl)propan-1-one |
| | LC-MS (Method 2): Rt = 1.28 min; MS (ESIpos): m/z = 536 [M + H]⁺ |
| | ¹H-NMR (400 MHz, DMSO-d₆): δ [ppm] = 1.51 (d, 3H), 2.17 (s, 3H), 3.20-3.31 |
| | (m, 4H), 3.86 (s, 3H), 3.95-4.04 (m, 2H), 4.36 (d, 1H), 4.67 (d, 1H), 4.78 (quin, |
| | 1H), 7.10-7.16 (m, 2H), 7.32 (t, 1H), 8.04 (dd, 1H), 8.19-8.25 (m, 2H), 8.27 |
| | (dd, 1H), 8.34 (br d, 1H). |
| Example 919 | 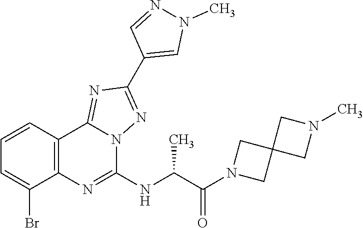 [SMILES: C[C@@H](Nc1nc2c(Br)cccc2c2nc(-c3cnn(C)c3)nn12)C(=O)N1CC2(CN(C)C2)C1] |
| | (2R)-2-{[7-bromo-2-(1-methyl-1H-pyrazol-4-yl)[1,2,4]triazolo[1,5- |
| | c]quinazolin-5-yl]amino}-1-(6-methyl-2,6-diazaspiro[3.3]heptan-2- |
| | yl)propan-1-one |
| | LC-MS (Method 2): Rt = 1.00 min; MS (ESIpos): m/z = 510 [M + H]⁺ |
| | ¹H-NMR (400 MHz, DMSO-d₆): δ [ppm] = 1.49 (d, 3H), 2.17 (s, 3H), 3.20-3.30 |
| | (m, 4H), 3.92-4.03 (m, 5H), 4.35 (br d, 1H), 4.64 (br d, 1H), 4.71-4.80 (m, |
| | 1H), 7.32 (t, 1H), 8.02-8.08 (m, 2H), 8.17 (br d, 1H), 8.23 (dd, 1H), 8.44 (s, |
| | 1H). |
| Example 920 | 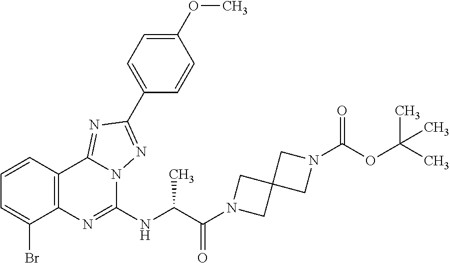 [SMILES: COc1ccc(-c2nc3c4cccc(Br)c4nc(N[C@H](C)C(=O)N4CC5(CN(C(=O)OC(C)(C)C)C5)C4)n3n2)cc1] |
| | tert-butyl 6-{N-[7-bromo-2-(4-methoxyphenyl)[1,2,4]triazolo[1,5- |
| | c]quinazolin-5-yl]-D-alanyl}-2,6-diazaspiro[3.3]heptane-2-carboxylate |
| | LC-MS (Method 2): Rt = 1.47 min; MS (ESIpos): m/z = 622 [M + H]⁺ |
| | ¹H-NMR (400 MHz, DMSO-d₆): δ [ppm] = 1.37 (s, 9H), 1.51 (br d, 3H), 3.86 (s, |
| | 3H), 3.94-34.14 (m, 6H), 4.44 (br d, 1H), 4.70-4.83 (m ,2H), 7.15 (br d, 2H), |
| | 7.34 (br t, 1H), 8.06 (br d, 1H), 8.24 (br d, 2H), 8.31 (br dd, 2H). |
| Example 921 | 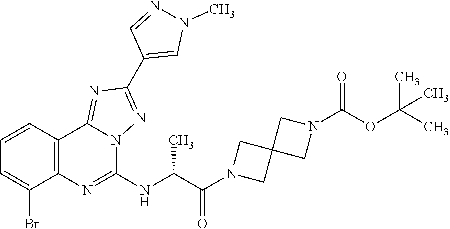 [SMILES: C[C@@H](Nc1nc2c(Br)cccc2c2nc(-c3cnn(C)c3)nn12)C(=O)N1CC2(CN(C(=O)OC(C)(C)C)C2)C1] |
| | tert-butyl 6-{N-[7-bromo-2-(1-methyl-1H-pyrazol-4-yl)[1,2,4]triazolo[1,5- |
| | c]quinazolin-5-yl]-D-alanyl}-2,6-diazaspiro[3.3]heptane-2-carboxylate |
| | LC-MS (Method 2): Rt = 1.23 min; MS (ESIpos): m/z = 596 [M + H]⁺ |
| | ¹H-NMR (400 MHz, DMSO-d₆): δ [ppm] = 1.37 (s, 9H), 1.49 (d, 3H), 3.92-4.12 |
| | (m, 9H), 4.43 (d, 1H), 4.71 (d, 1H), 4.76 (br d, 1H), 7.33 (t, 1H), 8.04-8.08 (m, |
| | 2H), 8.15 (br s, 1H), 8.24 (dd, 1H), 8.44 (s, 1H). |

### Example 922

### (2R)-2-{[7-bromo-2-(4-methoxyphenyl)[1,2,4]triazolo[1,5-c]quinazolin-5-yl]amino}-3-methyl-1-(4-methylpiperazin-1-yl)butan-1-one

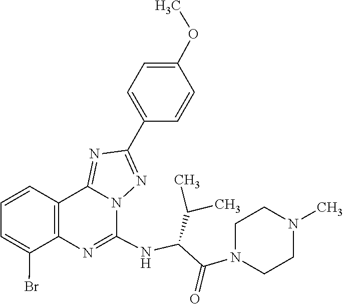 [SMILES: COc1ccc(-c2nc3c4cccc(Br)c4nc(N[C@@H](C(=O)N4CCN(C)CC4)C(C)C)n3n2)cc1]

N-[7-Bromo-2-(4-methoxyphenyl)[1,2,4]triazolo[1,5-c]quinazolin-5-yl]-D-valine (100 mg, 213 μmol), 1-methylpiperazine (47 μL, 430 μmol) and N,N-diisopropylethylamine (110 μL, 640 μmol) were solubilised in DMF (2.1 mL). T3P (150 μL, 50% in DMF, 260 μmol) was added and the reaction was stirred for 2 h at rt. The reaction mixture was diluted with half saturated aqueous sodium chloride solution. The precipitate was filtered, washed with water and hexane and dried to give 114 mg (95% purity, 92% yield) of the title compound.

LC-MS (method 2): Rₜ=1.48 min; MS (ESIpos): m/z=554 [M+H]⁺

1H-NMR (400 MHz, DMSO-d₆): δ [ppm]=1.01 (d, 3H), 1.07 (d, 3H), 2.18 (s, 3H), 2.22-2.47 (m, 5H), 3.45-3.54 (m, 1H), 3.54-3.65 (m, 1H), 3.81 (br t, 2H), 3.86 (s, 3H), 5.26 (t, 1H), 7.12 (d, 2H), 7.33 (s, 1H), 7.73 (d, 1H), 8.04 (d, 1H), 8.19-8.25 (m, 2H), 8.28 (dd, 1H).

### Example 923

### N²-[7-bromo-2-(4-methoxyphenyl)[1,2,4]triazolo[1,5-c]quinazolin-5-yl]-N-(2-methoxyethyl)-D-valinamide

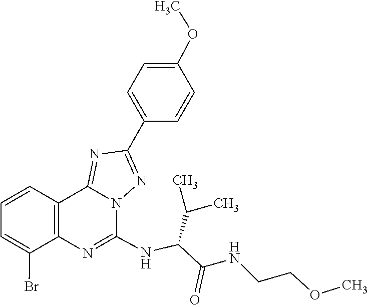 [SMILES: COCCNC(=O)[C@H](Nc1nc2c(Br)cccc2c2nc(-c3ccc(OC)cc3)nn12)C(C)C]

N-[7-Bromo-2-(4-methoxyphenyl)[1,2,4]triazolo[1,5-c]quinazolin-5-yl]-D-valine (100 mg, 213 μmol), 2-methoxyethan-1-amine (37 μL, 430 μmol) and N,N-diisopropylethylamine (110 μL, 640 μmol) were solubilised in DMF (2.1 mL). T3P (150 μL, 50% in DMF, 260 μmol) was added and the reaction was stirred for 2 h at rt. The reaction mixture was diluted with half saturated aqueous sodium chloride solution. The precipitate was filtered, washed with water and hexane and dried to give 104 mg (95% purity, 88% yield) of the title compound.

LC-MS (method 2): Rₜ=1.42 min; MS (ESIpos): m/z=529 [M+H]⁺

1H-NMR (400 MHz, DMSO-d₆): δ [ppm]=0.98-1.09 (m, 6H), 2.33-2.42 (m, 1H), 3.22 (s, 3H), 3.29 (br d, 2H), 3.37-3.43 (m, 3H), 3.86 (s, 3H), 4.76 (dd, 1H), 7.12-7.17 (m, 2H), 7.34 (t, 1H), 7.53 (d, 1H), 8.06 (dd, 1H), 8.22-8.27 (m, 2H), 8.29 (dd, 1H), 8.47 (t, 1H).

### Example 924

### N²-[7-bromo-2-(4-methoxyphenyl)[1,2,4]triazolo[1,5-c]quinazolin-5-yl]-N-[2-(4-methylpiperazin-1-yl)ethyl]-D-valinamide

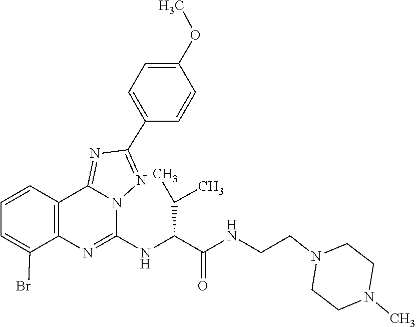 [SMILES: COc1ccc(-c2nc3c4cccc(Br)c4nc(N[C@@H](C(=O)NCCN4CCN(C)CC4)C(C)C)n3n2)cc1]

N-[7-Bromo-2-(4-methoxyphenyl)[1,2,4]triazolo[1,5-c]quinazolin-5-yl]-D-valine (100 mg, 213 μmol), 2-(4-methylpiperazin-1-yl)ethan-1-amine (64 μL, 430 μmol) and N,N-diisopropylethylamine (110 μL, 640 μmol) were solubilised in DMF (2.1 mL). T3P (150 μL, 50% in DMF, 260 μmol) was added and the reaction was stirred for 2 h at rt. The reaction mixture was diluted with half saturated aqueous sodium chloride solution. The precipitate was filtered, washed with water and hexane. The solid was suspended in cyclopentylmethylether and sonicated. The suspension was filtered and the filtrate was concentrated under reduced pressure. The crude material was suspended in hexane and few drops of dichlormethane were added. The suspension was sonicated and the solid was filtered, washed with hexane and dried to give 56.8 mg (95% purity, 43% yield) of the title compound without further purification.

LC-MS (method 2): Rₜ=1.36 min; MS (ESIneg): m/z=593 [M−H]⁻.

1H-NMR (400 MHz, DMSO-d₆): δ [ppm]=1.02 (d, 3H), 1.06 (d, 3H), 2.03-2.08 (m, 3H), 2.09-2.45 (m, 10H), 3.12-3.23 (m, 1H), 3.25-3.31 (m, 1H), 3.86 (s, 3H), 4.67 (dd, 1H), 7.12-7.17 (m, 2H), 7.34 (t, 1H), 7.63 (d, 1H), 8.06 (dd, 1H), 8.22-8.27 (m, 3H), 8.29 (dd, 1H).

### Example 925

### (2R)-2-{[7-bromo-2-(4-methoxyphenyl)[1,2,4]triazolo[1,5-c]quinazolin-5-yl]amino}-3-methyl-1-(morpholin-4-yl)butan-1-one

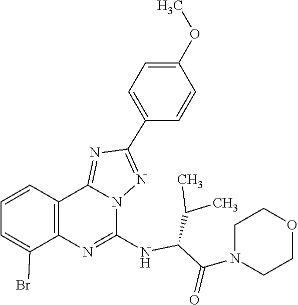 [SMILES: COc1ccc(-c2nc3c4cccc(Br)c4nc(N[C@@H](C(=O)N4CCOCC4)C(C)C)n3n2)cc1]

N-[7-Bromo-2-(4-methoxyphenyl)[1,2,4]triazolo[1,5-c]quinazolin-5-yl]-D-valine (100 mg, 213 μmol), morpholine (37 μL, 430 μmol; CAS-RN:[110-91-8]) and N,N-diisopropylethylamine (110 μL, 640 μmol) were solubilised in DMF (2.1 mL). T3P (150 μL, 50% in DMF, 260 μmol) was added and the reaction was stirred for 2 h at rt. The reaction mixture was diluted with half saturated aqueous sodium chloride solution. The precipitate was filtered, washed with water and hexane and dried to give 113 mg (95% purity, 93% yield) of the title compound without further purification.

LC-MS (method 2): Rₜ=1.49 min; MS (ESIpos): m/z=541 [M+H]⁺

1H-NMR (400 MHz, DMSO-d6): δ [ppm]=1.03 (d, 3H), 1.08 (d, 3H), 2.33-2.42 (m, 1H), 3.47-3.61 (m, 4H), 3.67 (br t, 2H), 3.86 (s, 5H), 5.22 (t, 1H), 7.10-7.15 (m, 2H), 7.34 (t, 1H), 7.76 (d, 1H), 8.06 (dd, 1H), 8.21-8.26 (m, 2H), 8.28 (dd, 1H).

### Example 926

### (2R)-2-{[7-bromo-2-(1-methyl-1H-pyrazol-4-yl)[1,2,4]triazolo[1,5-c]quinazolin-5-yl]amino}-3-methyl-1-(morpholin-4-yl)butan-1-one

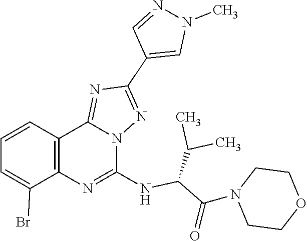 [SMILES: CC(C)[C@@H](Nc1nc2c(Br)cccc2c2nc(-c3cnn(C)c3)nn12)C(=O)N1CCOCC1]

N-[7-Bromo-2-(1-methyl-1H-pyrazol-4-yl)[1,2,4]triazolo[1,5-c]quinazolin-5-yl]-D-valine (81.0 mg, 182 μmol), morpholine (32 μL, 360 μmol) and N,N-diisopropylethylamine (95 μL, 550 μmol) were solubilised in DMF (1.8 mL). T3P (130 μL, 50% in DMF, 220 μmol) was added and the reaction was stirred for 2 h at rt. Morpholine (16 μL, 180 μmol) and T3P (65 μL, 50% in DMF, 110 μmol) were a added and the reaction was stirred another hour. The reaction mixture was diluted with half saturated aqueous sodium chloride solution and extracted with a mixture of ethyl acetate and some methanol The organic phase was dried (sodium sulfate), filtered and concentrated under reduced pressure. The crude material was suspended in hexane/diethyl ether and sonicated. The solid was filtered, washed and dried to give 70.6 mg (95% purity, 72% yield) of the title compound.

LC-MS (method 2): Rₜ=1.20 min; MS (ESIpos): m/z=515 [M+H]⁺

1H-NMR (400 MHz, DMSO-d6): δ [ppm]=1.01 (d, 3H), 1.08 (d, 3H), 2.26-2.37 (m, 1H), 3.48-3.59 (m, 4H), 3.62-3.69 (m, 2H), 3.74-3.87 (m, 2H), 3.95 (s, 3H), 5.22 (t, 1H), 7.33 (t, 1H), 7.51 (br d, 1H), 8.06 (dd, 1H), 8.09 (s, 1H), 8.24 (dd, 1H), 8.50 (s, 1H).

### Example 927

### N²-[7-bromo-2-(1-methyl-1H-pyrazol-4-yl)[1,2,4]triazolo[1,5-c]quinazolin-5-yl]-N-[2-(4-methylpiperazin-1-yl)ethyl]-D-valinamide

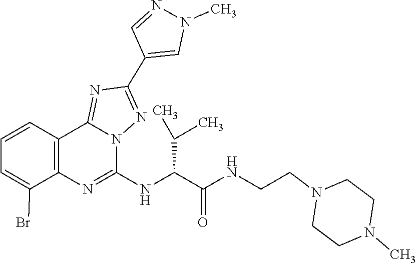 [SMILES: CC(C)[C@@H](Nc1nc2c(Br)cccc2c2nc(-c3cnn(C)c3)nn12)C(=O)NCCN1CCN(C)CC1]

N-[7-Bromo-2-(1-methyl-1H-pyrazol-4-yl)[1,2,4]triazolo[1,5-c]quinazolin-5-yl]-D-valine (81.0 mg, 182 μmol), 2-(4-methylpiperazin-1-yl)ethan-1-amine (55 μL, 360 μmol) and N,N-diisopropylethylamine (95 μL, 550 μmol) were solubilised in DMF (1.8 mL). T3P (130 μL, 50% in DMF, 220 μmol) was added and the reaction was stirred overnight at rt. 2-(4-Methylpiperazin-1-yl)ethan-1-amine (18 μL, 150 μmol) and T3P (75 μL, 50% in DMF, 110 μmol) were added again and the reaction was stirred for 1 h. The reaction mixture was diluted with half saturated aqueous sodium chloride solution and extracted with ethyl acetate and some methanol. The organic phase was dried (sodium sulfate), filtered and concentrated under reduced pressure. The crude material was suspended in hexane/diethyl ether and sonicated. The solid was filtered, washed and dried to give 66.4 mg (95% purity, 61% yield) of the title compound.

LC-MS (method 2): Rₜ=1.08 min; MS (ESIneg): m/z=567 [M−H]⁻.

1H-NMR (400 MHz, DMSO-d6): δ [ppm]=1.00 (d, 3H), 1.05 (d, 3H), 2.06 (s, 3H), 2.09-2.25 (m, 3H), 2.26-2.46 (m, 7H), 3.11-3.24 (m, 1H), 3.25-3.31 (m, 1H), 3.95 (s, 3H), 4.69 (dd, 1H), 7.33 (t, 1H), 7.49 (d, 1H), 8.05 (dd, 1H), 8.09 (s, 1H), 8.21-8.29 (m, 2H), 8.51 (s, 1H).

### Example 928

### (2R)-2-{[7-bromo-2-(1-methyl-1H-pyrazol-4-yl)[1,2,4]triazolo[1,5-c]quinazolin-5-yl]amino}-3-methyl-1-(4-methylpiperazin-1-yl)butan-1-one

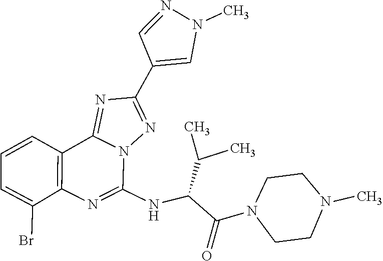 [SMILES: CC(C)[C@@H](Nc1nc2c(Br)cccc2c2nc(-c3cnn(C)c3)nn12)C(=O)N1CCN(C)CC1]

N-[7-Bromo-2-(1-methyl-1H-pyrazol-4-yl)[1,2,4]triazolo[1,5-c]quinazolin-5-yl]-D-valine (81.0 mg, 182 μmol), 1-methylpiperazine (40 μL, 360 μmol) and N,N-diisopropylethylamine (95 μL, 550 μmol) were solubilised in DMF (1.8 mL). T3P (130 μL, 50% in DMF, 220 μmol) was added and the reaction was stirred for 2 h at rt. The reaction mixture was diluted with half saturated aqueous sodium chloride solution and extracted with ethyl acetate and some methanol. The organic phase was dried (sodium sulfate), filtered and concentrated under reduced pressure. The crude material was suspended in hexane/dichloromethane and sonicated. The solid was filtered, washed and dried to give 68.8 mg (95% purity, 68% yield) of the title compound.

LC-MS (method 2): Rₜ=1.19 min; MS (ESIpos): m/z=528 [M+H]⁺

1H-NMR (400 MHz, DMSO-d6): δ [ppm]=0.99 (d, 3H), 1.07 (d, 3H), 2.15-2.24 (m, 3H), 2.24-2.47 (m, 5H), 3.44-3.54 (m, 1H), 3.57 (br s, 1H), 3.77 (br s, 2H), 3.95 (s, 3H), 5.25 (dd, 1H), 7.33 (t, 1H), 7.48 (d, 1H), 8.04-8.08 (m, 1H), 8.09 (s, 1H), 8.24 (dd, 1H), 8.50 (s, 1H).

### Example 929

### N²-[7-bromo-2-(1-methyl-1H-pyrazol-4-yl)[1,2,4]triazolo[1,5-c]quinazolin-5-yl]-N-(2-methoxyethyl)-D-valinamide

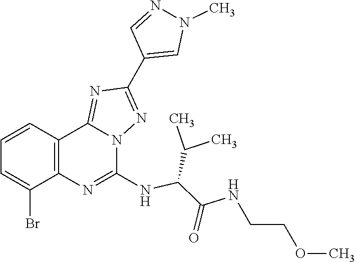 [SMILES: COCCNC(=O)[C@H](Nc1nc2c(Br)cccc2c2nc(-c3cnn(C)c3)nn12)C(C)C]

N-[7-Bromo-2-(1-methyl-1H-pyrazol-4-yl)[1,2,4]triazolo[1,5-c]quinazolin-5-yl]-D-valine (81.0 mg, 182 μmol), 2-methoxyethan-1-amine (32 μL, 360 μmol) and N,N-diisopropylethylamine (95 μL, 550 μmol) were solubilised in DMF (1.8 mL). T3P (130 μL, 50% purity, 220 μmol) was added and the reaction was stirred overnight at rt. 2-Methoxyethan-1-amine (16 μL, 180 μmol) and T3P (75 μL, 50% in DMF, 110 μmol) were added again and the reaction was stirred for 1 h. The reaction mixture was diluted with half saturated. aqueous sodium chloride solution and extracted with ethyl acetate and some methanol. The organic phase was dried (sodium sulfate), filtered and concentrated under reduced pressure. The crude material was suspended in hexane/diethyl ether and sonicated. The solid was filtered, washed and dried to give 67.8 mg (95% purity, 70% yield) of the title compound.

LC-MS (method 2): Rₜ=1.13 min; MS (ESIpos): m/z=503 [M+H]⁺

1H-NMR (400 MHz, DMSO-d6): δ [ppm]=0.98 (d, 3H), 1.04 (d, 3H), 2.29-2.41 (m, 1H), 3.22 (s, 3H), 3.25-3.32 (m, 2H), 3.35-3.40 (m, 2H), 3.95 (s, 3H), 4.78 (br t, 1H), 7.32 (t, 1H), 7.40 (br d, 1H), 8.03-8.07 (m, 1H), 8.09 (s, 1H), 8.24 (dd, 1H), 8.46-8.53 (m, 2H).

### Example 930

### 2-(1-methyl-1H-pyrazol-4-yl)-5-{[(6R)-5-oxo-1,4-diazepan-6-yl]amino}[1,2,4]triazolo[1,5-c]quinazoline-7-carbonitrile

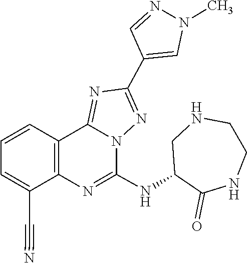 [SMILES: Cn1cc(-c2nc3c4cccc(C#N)c4nc(N[C@@H]4CNCCNC4=O)n3n2)cn1]

Benzyl (6R)-6-{[7-cyano-2-(1-methyl-1H-pyrazol-4-yl)[1,2,4]triazolo[1,5-c]quinazolin-5-yl]amino}-5-oxo-1,4-diazepane-1-carboxylate (180 mg, 335 μmol) was solubilised in dichloromethane (6.4 mL), hydrobromic acid in acetic acid (550 μL, 33%, 3.4 mmol) was added and the mixture was stirred over night at rt. The formed solid was obtain by filtration. The solid was stirred in methanol and basified with triethylamine. The resulting suspension was filtered, the obtained solid stirred in water (5 mL), again filtered, washed with water and dried to give 60.8 mg (95% purity, 43% yield) of the title compound.

Alternatively, in cases of high solubility in water the compounds could be purified by flash chromatography or preparative HPLC.

LC-MS (Method 2): Rₜ=0.79 min; MS (ESIneg): m/z=401 [M−H]⁻.

¹H-NMR (400 MHz, DMSO-d₆): δ [ppm]=2.51-2.61 (n, 1H, partial in DMSO signal), 2.61-2.70 (m, 1H, partialin DMSO signal), 2.75 (br dd, 1H), 3.01-3.17 (m, 2H), 3.37-3.51 (m, 2H), 3.95 (s, 3H), 4.88-4.94 (m, 1H), 7.52 (t, 1H), 7.98 (br d, 1H), 8.06 (s, 1H), 8.21 (dd, 1H), 8.29 (br dd, 1H), 8.45-8.52 (m, 2H).

The following examples were prepared similarly to example 930.

| | Structure |
|---|---|
| | IUPAC-Name |
| | LC-MS (method): Retention time; Mass found |
| Example | ¹H-NMR |
| Example 931 | 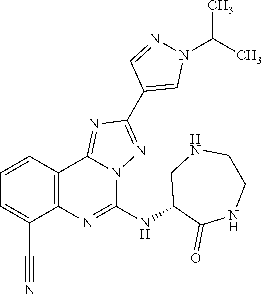 [SMILES: CC(C)n1cc(-c2nc3c4cccc(C#N)c4nc(N[C@@H]4CNCCNC4=O)n3n2)cn1] |
| | 5-{[(6R)-5-oxo-1,4-diazepan-6-yl]amino}-2-[1-(propan-2-yl)-1H-pyrazol-4- |
| | yl][1,2,4]triazolo[1,5-c]quinazoline-7-carbonitrile |
| | LC-MS (Method 2): Rₜ = 0.94 min; MS (ESIpos): m/z = 432 [M + H]⁺ |
| | ¹H-NMR (400 MHz, DMSO-d₆): δ [ppm] = 1.49 (d, 6H), 2.50-2.57 (m, 1H, |
| | partial in DMSO signal), 2.75 (dd, 2H), 3.02-3.17 (m, 2H), 3.36-3.51 (m, |
| | 2H), 4.64 (spt, 1H), 4.89-4.95 (m, 1H), 7.53 (t, 1H), 7.99 (d, 1H), 8.09 (d, |
| | 1H), 8.21 (dd, 1H), 8.29 (dd, 1H), 8.50 (dd, 1H), 8.53 (d, 1H). |
| Example 932 | 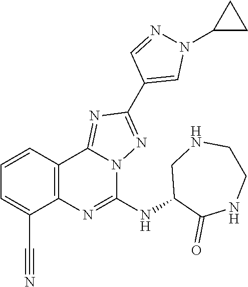 [SMILES: N#Cc1cccc2c1nc(N[C@@H]1CNCCNC1=O)n1nc(-c3cnn(C4CC4)c3)nc21] |
| | 2-(1-cyclopropyl-1H-pyrazol-4-yl)-5-{[(6R)-5-oxo-1,4-diazepan-6- |
| | yl]amino}[1,2,4]triazolo[1,5-c]quinazoline-7-carbonitrile |
| | LC-MS (Method 2): Rₜ = 0.91 min; MS (ESIpos): m/z = 429 [M + H]⁺ |
| | ¹H-NMR (400 MHz, DMSO-d₆): δ [ppm] = 0.99-1.05 (m, 2H), 1.15-1.20 (m, |
| | 2H), 2.51-2.58 (m, 1H, partial in DMSO signal), 2.61 (br s, 1H), 2.74 (dd, |
| | 1H), 3.01-3.18 (m, 2H), 3.36-3.43 (m, 1H), 3.47 (dd, 1H), 3.89 (tt, 1H), |
| | 4.91 (br d, 1H), 7.52 (t, 1H), 7.95-7.99 (m, 1H), 8.07 (d, 1H), 8.21 (dd, 1H), |
| | 8.29 (dd, 1H), 8.49 (dd, 1H), 8.55 (s, 1H). |
| Example 933 | 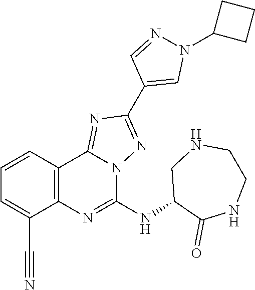 [SMILES: N#Cc1cccc2c1nc(N[C@@H]1CNCCNC1=O)n1nc(-c3cnn(C4CCC4)c3)nc21] |
| | 2-(1-cyclobutyl-1H-pyrazol-4-yl)-5-{[(6R)-5-oxo-1,4-diazepan-6- |
| | yl]amino}[1,2,4]triazolo[1,5-c]quinazoline-7-carbonitrile |
| | LC-MS (Method 1): Rₜ = 0.77 min; MS (ESIpos): m/z = 443 [M + H] |
| | ¹H-NMR (400 MHz, DMSO-d₆): δ [ppm] = 1.76-1.88 (m, 2H), 2.38-2.47 (m, |
| | 2H), 2.51-2.62 (m, 3H, partial in DMSO signal), 2.76 (dd, 2H), 3.01-3.18 |
| | (m, 2H), 3.36-3.51 (m, 2H), 4.89-5.02 (m, 2H), 7.53 (t, 1H), 7.99 (d, 1H), |
| | 8.12 (s, 1H), 8.21 (dd, 1H), 8.29 (dd, 1H), 8.50 (dd, 1H), 8.59 (s, 1H). |

### Example 934

### 2-[1-(difluoromethyl)-1H-pyrazol-4-yl]-5-{[(6R)-5-oxo-1,4-diazepan-6-yl]amino}[1,2,4]triazolo[1,5-c]quinazoline-7-carbonitrile

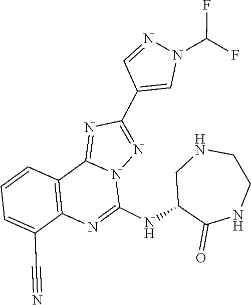 [SMILES: N#Cc1cccc2c1nc(N[C@@H]1CNCCNC1=O)n1nc(-c3cnn(C(F)F)c3)nc21]

A mixture of benzyl (6R)-6-({7-cyano-2-[1-(difluoromethyl)-1H-pyrazol-4-yl][1,2,4]triazolo[1,5-c]quinazolin-5-yl}amino)-5-oxo-1,4-diazepane-1-carboxylate and benzyl (6R)-6-{[7-cyano-2-(1H-pyrazol-4-yl)[1,2,4]triazolo[1,5-c]quinazolin-5-yl]amino}-5-oxo-1,4-diazepane-1-carboxylate (129 mg, about 225 μmol) was solubilised in dichloromethane (4.3 mL), hydrobromic acid in acetic acid (370 μL, 33%, 2.3 mmol) was added and the mixture was stirred over night at rt. The formed solid was obtain by filtration. The solid was stirred in methanol and basified with triethylamine. The resulting suspension was filtered, purification of the obtained solid by stirring in water failed because of a high solubility of the compounds in water. Therefore the complete material was purified via HPLC to obtain 18.4 mg (95% purity, 18% yield) of the title product and 27.8 mg (95% purity, 30% yield) of 5-{[(6R)-5-oxo-1,4-diazepan-6-yl]amino}-2-(1H-pyrazol-4-yl)[1,2,4]triazolo[1,5-c]quinazoline-7-carbonitrile.

LC-MS (Method 2): Rₜ=0.92 min; MS (ESIpos): m/z=439 [M+H]⁺

¹H-NMR (400 MHz, DMSO-d₆): δ [ppm]=2.52-2.58 (m, 1H, partial in DMSO signal), 2.61 (br s, 1H), 2.70-2.80 (m, 1H), 3.02-3.17 (m, 2H), 3.36-3.51 (m, 2H), 4.89-4.96 (m, 1H), 7.55 (t, 1H), 7.93 (t, 1H), 8.05 (br d, 1H), 8.24 (dd, 1H), 8.29 (dd, 1H), 8.45 (d, 1H), 8.52 (dd, 1H), 9.03 (d, 1H).

### Example 935

### 5-{[(6R)-5-oxo-1,4-diazepan-6-yl]amino}-2-(1H-pyrazol-4-yl)[1,2,4]triazolo[1,5-c]quinazoline-7-carbonitrile

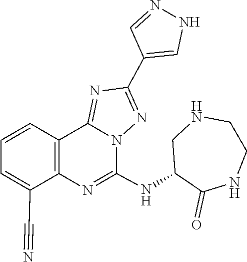 [SMILES: N#Cc1cccc2c1nc(N[C@@H]1CNCCNC1=O)n1nc(-c3cn[nH]c3)nc21]

A mixture of benzyl (6R)-6-({7-cyano-2-[1-(difluoromethyl)-1H-pyrazol-4-yl][1,2,4]triazolo[1,5-c]quinazolin-5-yl}amino)-5-oxo-1,4-diazepane-1-carboxylate and benzyl (6R)-6-{[7-cyano-2-(1H-pyrazol-4-yl)[1,2,4]triazolo[1,5-c]quinazolin-5-yl]amino}-5-oxo-1,4-diazepane-1-carboxylate (129 mg, about 225 μmol) was solubilised in dichloromethane (4.3 mL), hydrobromic acid in acetic acid (370 μL, 33%, 2.3 mmol) was added and the mixture was stirred over night at rt. The formed solid was obtain by filtration. The solid was stirred in methanol and basified with triethylamine. The resulting suspension was filtered, purification of the obtained solid by stirring in water failed because of a high solubility of the compounds in water. Therefore the complete material was purified via HPLC to obtain 27.8 mg (95% purity, 30% yield) of the title product and 18.4 mg (95% purity, 18% yield) of 2-[1-(difluoromethyl)-1H-pyrazol-4-yl]-5-{[(6R)-5-oxo-1,4-diazepan-6-yl]amino}[1,2,4]triazolo[1,5-c]quinazoline-7-carbonitrile.

LC-MS (Method 2): Rₜ=0.74 min; MS (ESIpos): m/z=389 [M+H]⁺

¹H-NMR (400 MHz, DMSO-d₆): δ [ppm]=2.52-2.57 (m, 1H), 2.64 (br s, 1H), 2.76 (dd, 1H), 3.01-3.17 (m, 2H), 3.36-3.52 (m, 2H), 4.89-4.96 (m, 1H), 7.52 (t, 1H), 8.00 (d, 1H), 8.17 (br s, 1H), 8.21 (dd, 1H), 8.28 (br dd, 1H), 8.38-8.61 (m, 2H), 13.39 (br s, 1H).

### Example 936

### (6R)-6-{[7-(methanesulfonyl)-2-(4-methoxyphenyl)[1,2,4]triazolo[1,5-c]quinazolin-5-yl]amino}-1,4-diazepan-5-one

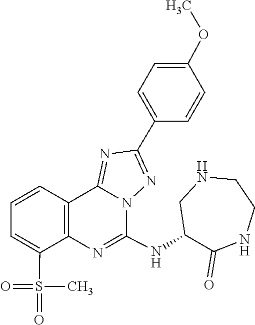 [SMILES: COc1ccc(-c2nc3c4cccc(S(C)(=O)=O)c4nc(N[C@@H]4CNCCNC4=O)n3n2)cc1]

A suspension of benzyl (6R)-6-{[7-(methanesulfonyl)-2-(4-methoxyphenyl)[1,2,4]triazolo[1,5-c]quinazolin-5-yl]amino}-5-oxo-1,4-diazepane-1-carboxylate (243 mg) in dichloromethane (5.0 mL) was treated with hydrobromic acid in acetic acid (700 μL, 33% purity, 3.9 mmol). The reaction was stirred at rt for 4 h. The reaction mixture was diluted with ethyl acetate and filtered. The solid was solubilised in dichloromethane/methanol and the mixture was basified with triethylamine. The mixture was concentrated under reduced pressure and purified by preparative HPLC to give 10 mg (100% purity, 5% yield) of the title compound.

LC-MS (Method 2): Rₜ=0.97 md; MS (ESIpos): m/z=482 [M+H]⁺

¹H-NMR (400 MHz, DMSO-d6): δ [ppm]=2.77 (br dd, 2H), 3.01-3.10 (m, 1H), 3.10-3.20 (m, 1H), 3.36-3.45 (m, 1H), 3.49 (br d, 1H), 3.61 (s, 3H), 3.86 (s, 3H), 4.90 (br d, 1H), 7.13-7.18 (m, 2H), 7.61 (t, 1H), 8.09 (br d, 1H), 8.21-8.27 (m, 2H), 8.30 (dd, 2H), 8.61 (dd, 1H).

The following compounds were prepared similarly to example 936.

| | Structure |
|---|---|
| | IUPAC-Name |
| | LC-MS (method): Retention time; Mass found |
| Example | ¹H-NMR |
| Example 937 | 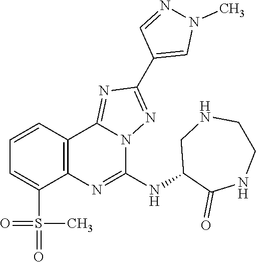 [SMILES: Cn1cc(-c2nc3c4cccc(S(C)(=O)=O)c4nc(N[C@@H]4CNCCNC4=O)n3n2)cn1] |
| | (6R)-6-{[7-(methanesulfonyl)-2-(1-methyl-1H-pyrazol-4- |
| | yl)[1,2,4]triazolo[1,5-c]quinazolin-5-yl]amino}-1,4-diazepan-5-one |
| | LC-MS (Method 2): Rₜ = 0.69 min; MS (ESIpos): m/z = 456 [M + H]⁺ |
| | ¹H-NMR (400 MHz, DMSO-d₆): δ [ppm] = 2.68-2.83 (m, 2H), 3.01-3.19 (m, |
| | 2H), 3.36-3.44 (m, 1H), 3.49 (br d, 1H), 3.61 (s, 3H), 3.96 (s, 3H), 4.88 (br |
| | d, 1H), 7.60 (t, 1H), 7.95 (br s, 1H), 8.08 (s, 1H), 8.27-8.36 (m, 2H), 8.49- |
| | 8.52 (m, 1H), 8.55 (dd, 1H). |
| Example 938 | 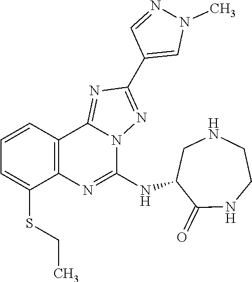 [SMILES: CCSc1cccc2c1nc(N[C@@H]1CNCCNC1=O)n1nc(-c3cnn(C)c3)nc21] |
| | (6R)-6-{[7-ethylsulfanyl)-2-(1-methyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.90 min; MS (ESIpos): m/z = 438 [M + H]⁺ |
| | ¹H-NMR (400 MHz, DMSO-d₆): δ [ppm] = 1.35 (t, 3H), 2.64-2.72 (m, 1H), |
| | 3.01-3.17 (m, 4H), 3.37-3.46 (m, 1H), 3.47-3.52 (m, 1H), 3.95 (s, 3H), |
| | 4.79-4.86 (m, 1H), 7.40 (t, 1H), 7.57 (dd, 1H), 7.64 (d, 1H), 7.99 (dd, 1H), |
| | 8.06 (s, 1H), 8.28 (dd, 1H), 8.48 (s, 1H). |
| Example 939 | 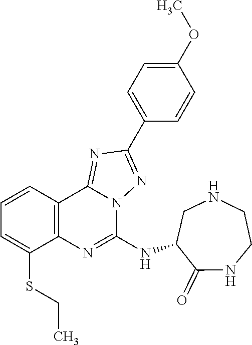 [SMILES: CCSc1cccc2c1nc(N[C@@H]1CNCCNC1=O)n1nc(-c3ccc(OC)cc3)nc21] |
| | (6R)-6-{[7-(ethylsulfanyl)-2-(4-methoxyphenyl)[1,2,4]triazolo[1,5- |
| | c]quinazolin-5-yl]amino}-1,4-diazepan-5-one |
| | LC-MS (Method 2): Rₜ = 1.18 min; MS (ESIpos): m/z = 464 [M + H]⁺ |
| | ¹H-NMR (400 MHz, DMSO-d₆): δ [ppm] = 1.36 (t, 3H), 2.53-2.57 (m, 1H), |
| | 2.65-2.69 (m, 1H), 2.70-2.76 (m, 1H), 3.03-3.05 (m, 1H), 3.07 (br d, 2H), |
| | 3.10-3.17 (m, 1H), 3.38-3.46 (m, 1H), 3.50 (br d, 1H), 3.86 (s, 3H), 4.81- |
| | 4.88 (m, 1H), 7.14 (d, 2H), 7.41 (t, 1H), 7.56-7.60 (m, 1H), 7.75 (d, 1H), |
| | 8.04 (dd, 1H), 8.22 (d, 2H), 8.28 (br dd, 1H). |
| Example 940 | 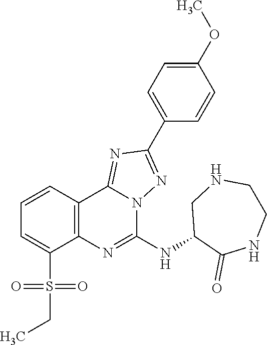 [SMILES: CCS(=O)(=O)c1cccc2c1nc(N[C@@H]1CNCCNC1=O)n1nc(-c3ccc(OC)cc3)nc21] |
| | (6R)-6-{[7-(ethanesulfonyl)-2-(4-methoxyphenyl)[1,2,4]triazolo[1,5- |
| | c]quinazolin-5-yl]amino}-1,4-diazepan-5-one |
| | LC-MS (Method 2): Rₜ = 1.05 min; MS (ESIpos): m/z = 496 [M + H]⁺ |
| | ¹H-NMR (400 MHz, DMSO-d₆): δ [ppm] = 1.16 (t, 3H), 2.78 (br dd, 1H), 3.05 |
| | (br dd, 1H), 3.11-3.20 (m, 1H), 3.43 (br d, 1H), 3.80-3.89 (m, 5H), 4.83- |
| | 4.93 (m, 1H), 7.16 (d, 2H), 7.62 (t, 1H), 8.11 (br d, 1H), 8.20-8.35 (m, 4H), |
| | 8.62 (dd, 1H). |
| Example 941 | 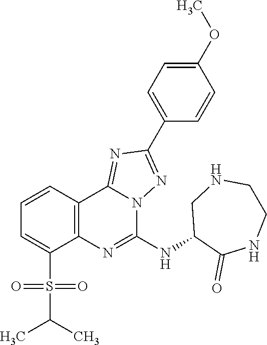 [SMILES: COc1ccc(-c2nc3c4cccc(S(=O)(=O)C(C)C)c4nc(N[C@@H]4CNCCNC4=O)n3n2)cc1] |
| | (6R)-6-{[2-(4-methoxyphenyl)-7-(propan-2-sulfonyl)[1,2,4]triazolo[1,5- |
| | c]quinazolin-5-yl]amino}-1,4-diazepan-5-one |
| | LC-MS (Method 2): Rₜ = 1.08 min; MS (ESIpos): m/z = 510 [M + H]⁺ |
| | ¹H-NMR (400 MHz, DMSO-d₆): δ [ppm] = 1.13 (d, 3H), 1.30 (d, 3H), 2.54- |
| | 2.60 (m, 1H), 2.81 (br dd, 1H), 3.05 (br dd, 1H), 3.10-3.24 (m, 1H), 3.37- |
| | 3.48 (m, 1H), 3.86 (s, 3H), 4.52 (quin, 1H), 4.82-4.94 (m, 1H), 7.12-7.22 |
| | (m, 2H), 7.56-7.66 (m, 1H), 8.15 (br d, 1H), 8.21-8.32 (m, 4H), 8.61 (dd, |
| | 1H). |
| Example 942 | 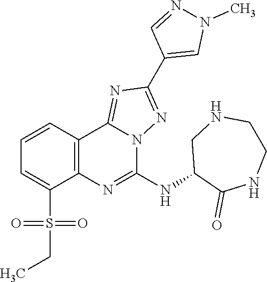 [SMILES: CCS(=O)(=O)c1cccc2c1nc(N[C@@H]1CNCCNC1=O)n1nc(-c3cnn(C)c3)nc21] |
| | (6R)-6-{[7-(ethanesulfonyl)-2-(1-methyl-1H-pyrazol-4- |
| | yl)[1,2,4]triazolo[1,5-c]quinazolin-5-yl]amino}-1,4-diazepan-5-one |
| | LC-MS (Method 2): Rₜ = 0.77 min; MS (ESIpos): m/z = 470 [M + H]⁺ |
| | ¹H-NMR (400 MHz, DMSO-d₆): δ [ppm] = 1.15 (t, 3H), 2.75 (br dd, 1H), 3.05 |
| | (br dd, 1H), 3.10-3.22 (m, 1H), 3.36-3.48 (m, 2H), 3.84 (q, 2H), 3.93-4.00 |
| | (m, 3H), 4.80-4.98 (m, 1H), 7.61 (t, 1H), 7.98 (br d, 1H), 8.09 (s, 1H), 8.26- |
| | 8.35 (m, 2H), 8.51 (s, 1H), 8.56 (dd, 1H). |
| Example 943 | 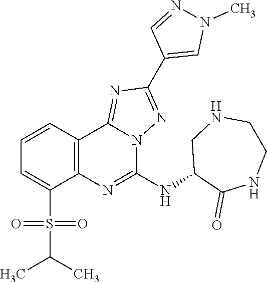 [SMILES: CC(C)S(=O)(=O)c1cccc2c1nc(N[C@@H]1CNCCNC1=O)n1nc(-c3cnn(C)c3)nc21] |
| | (6R)-6-{[2-(1-methyl-1H-pyrazol-4-yl)-7-(propane-2- |
| | sulfonyl)[1,2,4]triazolo[1,5-c]quinazolin-5-yl]amino}-1,4-diazepan-5-one |
| | LC-MS (Method 2): Rₜ = 0.81 min; MS (ESIpos): m/z = 484 [M + H]⁺ |
| | ¹H-NMR (400 MHz, DMSO-d₆): δ [ppm] = 1.14 (d, 3H), 1.29 (d, 3H), 2.73- |
| | 2.84 (m, 1H), 3.04 (br dd, 1H), 3.10-3.22 (m, 1H), 3.96 (s, 3H), 4.53 (quin, |
| | 1H), 4.79-4.93 (m, 1H), 7.61 (t, 1H), 8.02 (br d, 1H), 8.09 (s, 1H), 8.25- |
| | 8.32 (m, 2H), 8.50 (s, 1H), 8.56 (dd, 1H). |
| Example 944 | 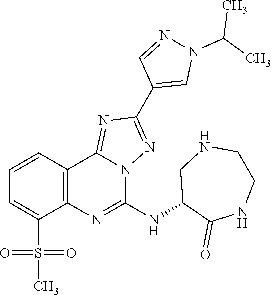 [SMILES: CC(C)n1cc(-c2nc3c4cccc(S(C)(=O)=O)c4nc(N[C@@H]4CNCCNC4=O)n3n2)cn1] |
| | (6R)-6-({7-(methanesulfonyl)-2-[1-(propan-2-yl)-1H-pyrazol-4- |
| | yl][1,2,4]triazolo[1,5-c]quinazolin-5-yl}amino)-1,4-diazepan-5-one |
| | LC-MS (Method 2): Rₜ = 0.87 min; MS (ESIpos): m/z = 484 [M + H]⁺ |
| | ¹H-NMR (400 MHz, DMSO-d₆): δ [ppm] = 1.50 (d, 6H), 2.52-2.57 (m, 1H), |
| | 2.75 (br dd, 1H), 3.06 (br dd, 1H), 3.10-3.22 (m, 1H), 3.38-3.45 (m, 1H), |
| | 3.49 (br d, 1H), 3.61 (s, 3H), 4.65 (spt, 1H), 4.90 (br ddd, 1H), 7.61 (t, 1H), |
| | 7.97 (d, 1H), 8.11 (s, 1H), 8.27-8.35 (m, 2H), 8.54-8.58 (m, 2H). |
| Example 945 | 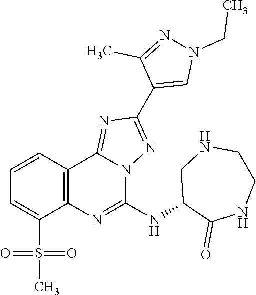 [SMILES: CCn1cc(-c2nc3c4cccc(S(C)(=O)=O)c4nc(N[C@@H]4CNCCNC4=O)n3n2)c(C)n1] |
| | (6R)-6-{[2-(1-ethyl-3-methyl-1H-pyrazol-4-yl)-7- |
| | (methanesulfonyl)[1,2,4]triazolo[1,5-c]quinazolin-5-yl]amino}-1,4- |
| | diazepan-5-one |
| | LC-MS (Method 2): Rₜ = 0.85 min; MS (ESIpos): m/z = 484 [M + H]⁺ |
| | ¹H-NMR (400 MHz, DMSO-d₆): δ [ppm] = 1.42 (t, 3H), 2.55 (br d, 1H), 2.60 (s, |
| | 3H), 2.76 (br dd, 1H), 3.06 (br dd, 1H), 3.11-3.21 (m, 1H), 3.38-3.47 (m, |
| | 1H), 3.51 (br d, 1H), 3.62 (s, 3H), 4.17 (q, 2H), 4.89 (br dd, 1H), 7.60 (t, 1H), |
| | 7.98 (d, 1H), 8.27-8.35 (m, 2H), 8.43 (s, 1H), 8.54 (dd, 1H). |
| Example 946 | 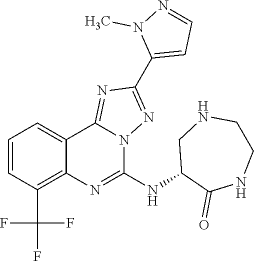 [SMILES: Cn1nccc1-c1nc2c3cccc(C(F)(F)F)c3nc(N[C@@H]3CNCCNC3=O)n2n1] |
| | (6R)-6-{[2-(1-methyl-1H-pyrazol-5-yl)-7- |
| | (trifluoromethyl)[1,2,4]triazolo[1,5-c]quinazolin-5-yl]amino}-1,4- |
| | diazepan-5-one |
| | LC-MS (Method 2): Rₜ = 1.04 min; MS (ESIpos): m/z = 446 [M + H]⁺ |
| | ¹H-NMR (400 MHz, DMSO-d₆): δ [ppm] = 2.75 (dd, 1H), 3.05 (br dd, 1H), 3.09- |
| | 3.17 (m, 1H), 3.35-3.41 (m, 1H), 3.47 (dd, 1H), 4.35 (s, 3H), 4.82-4.88 (m, |
| | 1H), 7.04 (d, 1H), 7.56 (t, 1H), 7.62 (d, 1H), 8.05 (d, 1H), 8.12 (dd, 1H), 8.26 |
| | (dd, 1H), 8.55 (dd, 1H). |
| Example 947 | 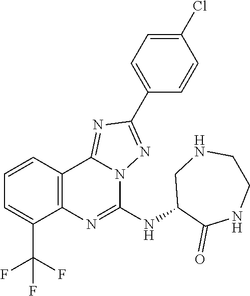 [SMILES: O=C1NCCNC[C@H]1Nc1nc2c(C(F)(F)F)cccc2c2nc(-c3ccc(Cl)cc3)nn12] |
| | (6R)-6-{[2-(4-chlorophenyl)-7-(trifluoromethyl)[1,2,4]triazolo[1,5- |
| | c]quinazolin-5-yl]amino}-1,4-diazepan-5-one |
| | LC-MS (Method 2): Rₜ = 1.37 min; MS (ESIpos): m/z = 476 [M + H]⁺ |
| | ¹H-NMR (400 MHz, DMSO-d₆): δ [ppm] = 2.62-2.84 (m, 1H), 2.97-3.22 (m, |
| | 2H), 3.48 (dd, 1H), 4.84 (br dd, 1H), 7.56 (t, 1H), 7.63-7.72 (m, 2H), 8.00 |
| | (d, 1H), 8.08-8.17 (m, 1H), 8.24-8.37 (m, 3H), 8.55 (dd, 1H). |
| Example 948 | 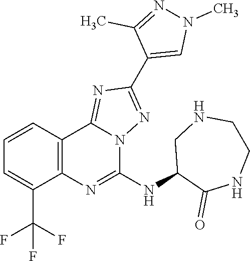 [SMILES: Cc1nn(C)cc1-c1nc2c3cccc(C(F)(F)F)c3nc(N[C@H]3CNCCNC3=O)n2n1] |
| | (6S)-6-{[2-(1,3-dimethyl-1H-pyrazol-4-yl)-7- |
| | trifluoromethyl)[1,2,4]triazolo[1,5-c]quinazolin-5-yl]amino}-1,4- |
| | diazepan-5-one |
| | LC-MS (Method 2): Rₜ = 1.03 min; MS (ESIpos): m/z = 460 [M + H]⁺ |
| | ¹H-NMR (400 MHz, DMSO-d₆): δ [ppm] = 2.52-2.54 (m, 1H), 2.58 (d, 3H), |
| | 2.64-2.75 (m, 1H), 3.05 (br dd, 1H), 3.08-3.17 (m, 1H), 3.40 (br d, 1H), |
| | 3.50 (dd, 1H), 3.87 (s, 3H), 4.81 (br dd, 1H), 7.54 (t, 1H), 7.85 (d, 1H), 8.06- |
| | 8.12 (m, 1H), 8.28 (dd, 1H), 8.38 (s, 1H), 8.49 (dd, 1H). |
| Example 949 | 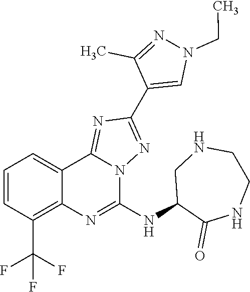 [SMILES: CCn1cc(-c2nc3c4cccc(C(F)(F)F)c4nc(N[C@H]4CNCCNC4=O)n3n2)c(C)n1] |
| | (6S)-6-{[2-(1-ethyl-3-methyl-1H-pyrazol-4-yl)-7- |
| | (trifluoromethyl)[1,2,4]triazolo[1,5-c]quinazolin-5-yl]amino}-1,4- |
| | diazepan-5-one |
| | LC-MS (Method 2): Rₜ = 1.05 min; MS (ESIpos): m/z = 474 [M + H]⁺ |
| | ¹H-NMR (400 MHz, DMSO-d₆): δ [ppm] = 1.42 (t, 3H), 2.60 (s, 3H), 2.62- |
| | 2.68 (m, 1H), 2.86-2.76 (m, 1H), 2.99-3.08 (m, 1H), 3.12 (ddd, 1H), 3.36- |
| | 3.43 (m, 1H), 3.44-3.56 (m, 1H), 4.17 (q, 2H), 4.81 (br dd, 1H), 7.54 (t, 1H), |
| | 7.85 (d, 1H), 8.09 (d, 1H), 8.28 (dd, 1H), 8.42 (s, 1H), 8.49 (dd, 1H). |
| Example 950 | 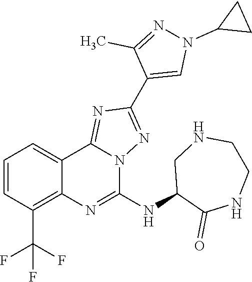 [SMILES: Cc1nn(C2CC2)cc1-c1nc2c3cccc(C(F)(F)F)c3nc(N[C@H]3CNCCNC3=O)n2n1] |
| | (6S)-6-{[2-(1-cyclopropyl-1H-pyrazol-4-yl)-7- |
| | (trifluoromethyl)[1,2,4]triazolo[1,5-c]quinazolin-5-yl]amino}-1,4- |
| | diazepan-5-one |
| | LC-MS (Method 2): Rₜ = 1.02 min; MS (ESIpos): m/z = 472 [M + H]⁺ |
| | ¹H-NMR (400 MHz, DMSO-d₆): δ [ppm] = 0.99-1.06 (m, 2H), 1.14-1.22 (m, |
| | 2H), 2.53-2.61 (m, 1H), 2.64-2.74 (m, 1H), 3.05 (br d, 1H), 3.08-3.17 (m, |
| | 1H), 3.36-3.42 (m, 1H), 3.48 (br d, 1H), 3.89 (tt, 1H), 4.82 (br d, 1H), 7.54 |
| | (t, 1H), 7.82 (br s, 1H), 8.05-8.12 (m, 2H), 8.29 (dd, 1H), 8.49 (d, 1H), 8.56 |
| | (s, 1H). |
| Example 951 | 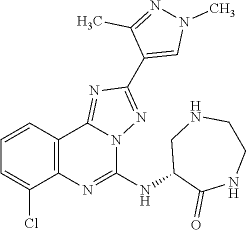 [SMILES: Cc1nn(C)cc1-c1nc2c3cccc(Cl)c3nc(N[C@@H]3CNCCNC3=O)n2n1] |
| | (6R)-6-{[7-chloro-2-(1,3-dimethyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.95 min; MS (ESIpos): m/z = 426 [M + H]⁺ |
| | ¹H-NMR (400 MHz, DMSO-d₆): δ [ppm] = 2.54 (s, 1H), 2.57 (s, 3H), 2.64- |
| | 2.74 (m, 1H), 2.75-2.97 (m, 1H), 2.99-3.07 (m, 1H), 3.07-3.18 (m, 1H), |
| | 3.37-3.47 (m, 1H), 3.53 (dd, 1H), 3.86 (s, 3H), 4.82-4.91 (m, 1H), 7.39 (t, |
| | 1H), 7.76 (d, 1H), 7.88 (dd, 1H), 8.18 (dd, 1H), 8.29 (dd, 1H), 8.36 (s, 1H). |
| Example 952 | 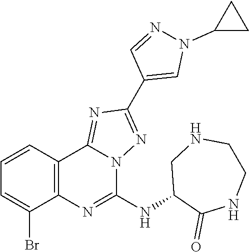 [SMILES: O=C1NCCNC[C@H]1Nc1nc2c(Br)cccc2c2nc(-c3cnn(C4CC4)c3)nn12] |
| | (6R)-6-{[7-bromo-2-(1-cyclopropyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.97 min; MS (ESIpos): m/z = 482 [M + H]⁺ |
| | ¹H-NMR (400 MHz, DMSO-d₆): δ [ppm] = 1.00-1.04 (m, 2H), 1.16-1.19 (m, |
| | 2H), 2.72-2.83 (m, 1H), 3.00-3.12 (m, 1H), 3.24 (br d, 2H), 3.43 (td, 1H), |
| | 3.51-3.64 (m, 1H), 3.70 (br d, 1H), 3.89 (tt, 1H), 5.02-5.10 (m, 1H), 7.35 (t, |
| | 1H), 7.97-8.04 (m, 1H), 8.04-8.12 (m, 2H), 8.26 (dd, 1H), 8.35-8.44 (m, |
| | 1H), 8.54 (s, 1H). |
| Example 953 | 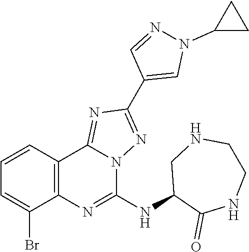 [SMILES: O=C1NCCNC[C@@H]1Nc1nc2c(Br)cccc2c2nc(-c3cnn(C4CC4)c3)nn12] |
| | (6S)-6-{[7-bromo-2-(1-cyclopropyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.97 min; MS (ESIpos): m/z = 482 [M + H]⁺ |
| | ¹H-NMR (400 MHz, DMSO-d₆): δ [ppm] = 1.00-1.04 (m, 2H), 1.16-1.19 (m, |
| | 2H), 2.71-2.83 (m, 1H), 2.98-3.17 (m, 1H), 3.22-3.28 (m, 2H), 3.38-3.49 |
| | (m, 1H), 3.50-3.61 (m, 1H), 3.70 (br d, 1H), 3.89 (tt, 1H), 5.01-5.10 (m, |
| | 1H), 7.35 (t, 1H), 7.97-8.04 (m, 1H), 8.04-8.11 (m, 2H), 8.26 (dd, 1H), |
| | 8.35-8.43 (m, 1H), 8.54 (s, 1H). |
| Example 954 | 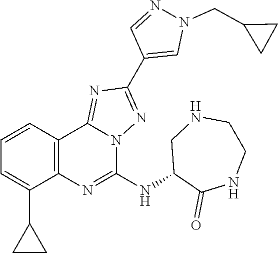 [SMILES: O=C1NCCNC[C@H]1Nc1nc2c(C3CC3)cccc2c2nc(-c3cnn(CC4CC4)c3)nn12] |
| | (6R)-6-({7-cyclopropyl-2-[1-(cyclopropylmethyl)-1H-pyrazol-4- |
| | yl][1,2,4]triazolo[1,5-c]quinazolin-5-yl}amino)-1,4-diazepan-5-one |
| | LC-MS (Method 2): Rₜ = 1.07 min; MS (ESIpos): m/z = 458 [M + H]⁺ |
| | ¹H-NMR (400 MHz, DMSO-d₆): δ [ppm] = 0.44 (dd, 2H), 0.53-0.61 (m, 2H), |
| | 0.78 (br dd, 1H), 0.84-0.91 (m, 1H), 1.10 (dd, 2H), 1.29-1.39 (m, 1H), 2.61- |
| | 2.70 (m, 1H), 2.88 (br d, 1H), 2.97-3.06 (m, 2H), 3.07-3.16 (m, 1H), 3.41 |
| | (ddd, 1H), 3.52 (br d, 1H), 4.08 (d, 2H), 4.84-4.92 (m, 1H), 7.23 (dd, 1H), |
| | 7.29-7.36 (m, 1H), 7.52 (d, 1H), 8.04 (dd, 1H), 8.08 (s, 1H), 8.30 (dd, 1H), |
| | 8.54 (d, 1H). |
| Example 955 | 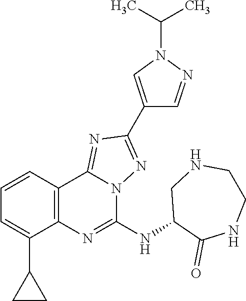 [SMILES: CC(C)n1cc(-c2nc3c4cccc(C5CC5)c4nc(N[C@@H]4CNCCNC4=O)n3n2)cn1] |
| | (6R)-6-({7-cyclopropyl-2-[1-(propan-2-yl)-1H-pyrazol-4- |
| | yl][1,2,4]triazolo[1,5-c]quinazolin-5-yl}amino)-1,4-diazepan-5-one |
| | LC-MS (Method 2): Rt = 1.06 min; MS (ESIpos): m/z = 446 [M + H]⁺ |
| | ¹H-NMR (400 MHz, DMSO-d₆): δ [ppm] = 0.71-0.96 (m, 3H), 1.10 (dd, 2H), |
| | 1.18-1.33 (m, 1H), 1.49 (d, 6H), 2.86 (br s, 1H), 2.96-3.05 (m, 2H), 3.11 |
| | (br s, 1H), 3.37-3.46 (m, 1H), 3.52 (br d, 1H), 4.63 (spt, 1H), 4.82-4.92 (m, |
| | 1H), 7.23 (dd, 1H), 7.29-7.37 (m, 1H), 7.51 (br d, 1H), 8.04 (dd, 1H), 8.08 |
| | (s, 1H), 8.30 (br dd, 1H), 8.52 (s, 1H). |
| Example 956 | 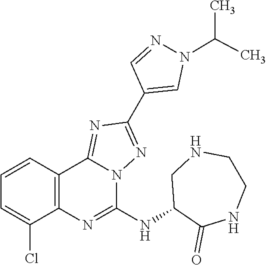 [SMILES: CC(C)n1cc(-c2nc3c4cccc(Cl)c4nc(N[C@@H]4CNCCNC4=O)n3n2)cn1] |
| | (6R)-6-({7-chloro-2-[1-(propan-2-yl)-1H-pyrazol-4-yl][1,2,4]triazolo[1,5- |
| | c]quinazolin-5-yl}amino)-1,4-diazepan-5-one |
| | LC-MS (Method 2): Rₜ = 1.02 min; MS (ESIpos): m/z = 440 [M + H]⁺ |
| | ¹H-NMR (400 MHz, DMSO-d₆): δ [ppm] = 1.49 (d, 6H), 2.52-2.55 (m, 1H), |
| | 2.65-2.73 (m, 1H), 3.04 (br dd, 1H), 3.07-3.17 (m, 1H), 3.37-3.47 (m, |
| | 1H), 3.51 (dd, 1H), 4.58-4.70 (m, 1H), 4.64 (quin, 1H), 4.89 (br dd, 1H), |
| | 7.40 (t, 1H), 7.74 (d, 1H), 7.89 (dd, 1H), 8.08 (s, 1H) 8.20 (dd, 1H), 8.30 |
| | (dd, 1H), 8.53 (s, 1H). |
| Example 957 | 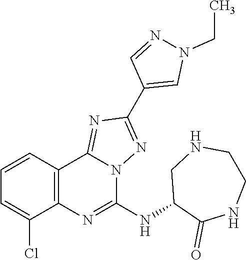 [SMILES: CCn1cc(-c2nc3c4cccc(Cl)c4nc(N[C@@H]4CNCCNC4=O)n3n2)cn1] |
| | (6R)-6-{[7-chloro-2-(1-ethyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.96 min; MS (ESIpos): m/z = 426 [M + H]⁺ |
| | ¹H-NMR (400 MHz, DMSO-d₆): δ [ppm] = 1.44 (t, 4H), 2.63-2.72 (m, 1H), |
| | 3.03 (br dd, 1H), 3.07-3.16 (m, 1H), 3.51 (dd, 1H) ,4.21-4.30 (m, 2H), 4.89 |
| | (br d, 1H), 7.40 (t, 2H), 7.74 (d, 1H), 7.89 (dd, 1H), 8.07-8.10 (m, 1H), 8.20 |
| | (dd, 1H), 8.30 (dd, 1H), 8.53 (s, 1H). |
| Example 958 | 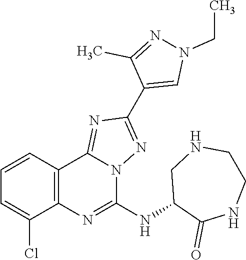 [SMILES: CCn1cc(-c2nc3c4cccc(Cl)c4nc(N[C@@H]4CNCCNC4=O)n3n2)c(C)n1] |
| | (6R)-6-{[7-chloro-2-(1-ethyl-3-methyl-1H-pyrazol-4-yl)[1,2,4]triazolo[1,5- |
| | c]quinazolin-5-yl]amino}-1,4-diazepan-5-one |
| | LC-MS (Method 2): Rₜ = 1.02 min; MS (ESIpos): m/z = 440 [M + H]⁺ |
| | ¹H-NMR (400 MHz, DMSO-d₆): δ [ppm] = 1.41 (t, 3H), 2.52-2.56 (m, 1H), |
| | 2.59 (s, 3H), 2.70 (dd, 1H), 3.04 (br dd, 1H), 3.08-3.18 (m, 1H), 3.42 (br s, |
| | 1H), 3.53 (dd, 1H), 4.16 (q, 2H), 4.88 (ddd, 1H), 7.40 (t, 1H), 7.77 (d, 1H), |
| | 7.88 (dd, 1H), 8.19 (dd, 1H), 8.29 (dd, 1H), 8.41 (s, 1H). |
| Example 959 | 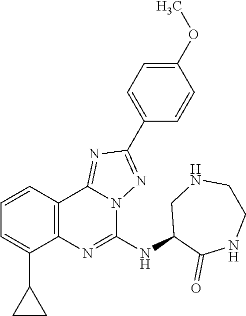 [SMILES: COc1ccc(-c2nc3c4cccc(C5CC5)c4nc(N[C@H]4CNCCNC4=O)n3n2)cc1] |
| | (6S)-6-{[7-cyclopropyl-2-(4-methoxyphenyl)[1,2,4]triazolo[1,5- |
| | c]quinazolin-5-yl]amino}-1,4-diazepan-5-one |
| | LC-MS (Method 2): Rₜ = 1.21 min; MS (ESIpos): m/z = 444 [M + H]⁺ |
| | ¹H-NMR (400 MHz, DMSO-d₆): δ [ppm] = 0.76-0.82 (m, 1H), 0.84-0.92 (m, |
| | 1H), 1.11 (dd, 2H), 2.52-2.56 (m, 1H), 2.65-2.74 (m, 1H), 2.97-3.07 (m, |
| | 2H), 3.08-3.18 (m, 1H), 3.18-3.28 (m, 1H), 3.41-3.49 (m, 1H), 3.54 (dd, |
| | 1H), 3.86 (s, 3H), 4.90 (ddd, 1H), 7.12-7.18 (m, 2H), 7.23 (dd, 1H), 7.31- |
| | 7.38 (m, 1H), 7.63 (d, 1H), 8.09 (dd, 1H), 8.20-8.25 (m, 2H), 8.26-8.34 (m, |
| | 1H). |
| Example 960 | 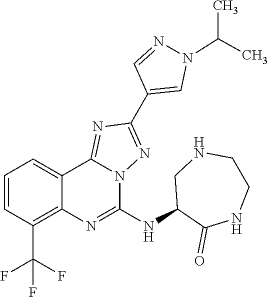 [SMILES: CC(C)n1cc(-c2nc3c4cccc(C(F)(F)F)c4nc(N[C@H]4CNCCNC4=O)n3n2)cn1] |
| | (6S)-6-({2-[1-(propan-2-yl)-1H-pyrazol-4-yl]-7- |
| | (trifluoromethyl)[1,2,4]triazolo[1,5-c]quinazolin-5-yl}amino)-1,4- |
| | diazepan-5-one |
| | LC-MS (Method 2): Rₜ = 1.06 min; MS (ESIpos): m/z = 474 [M + H]⁺ |
| | ¹H-NMR (400 MHz, DMSO-d₆): δ [ppm] = 1.50 (d, 6H), 2.53 (br s, 1H), 2.68- |
| | 2.77 (m, 1H), 2.99-3.09 (m, 1H), 3.09-3.18 (m, 1H), 3.39-3.44 (m, 1H), |
| | 3.49 (br d, 1H), 4.65 (spt, 1H), 4.81-4.87 (m, 1H), 7.55 (t, 1H), 7.86 (br d, |
| | 1H), 8.08-8.12 (m, 2H), 8.30 (dd, 1H), 8.49-8.55 (m, 2H). |
| Example 961 | 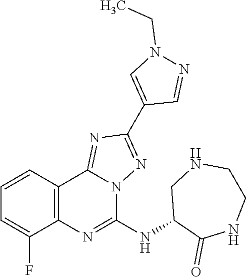 [SMILES: CCn1cc(-c2nc3c4cccc(F)c4nc(N[C@@H]4CNCCNC4=O)n3n2)cn1] |
| | (6R)-6-{[2-(1-ethyl-1H-pyrazol-4-yl)-7-fluoro-[1,2,4]triazolo[1,5- |
| | c]quinazolin-5-yl]amino}1-4,-diazepan-5-one |
| | LC-MS (Method 2): Rₜ = 0.87 min; MS (ESIpos): m/z = 410 [M + H]⁺ |
| | ¹H-NMR (400 MHz, DMSO-d₆): δ [ppm] = 1.45 (t, 3H), 2.61-2.75 (m, 1H), |
| | 2.97-3.19 (m, 2H), 3.38-3.51 (m, 2H), 4.25 (q, 2H), 4.87 (br dd, 1H), 7.41 |
| | (td, 1H), 7.60 (ddd, 1H), 7.73 (d, 1H), 7.96-8.15 (m, 2H), 8.31 (dd, 1H), |
| | 8.53 (s, 1H). |
| Example 962 | 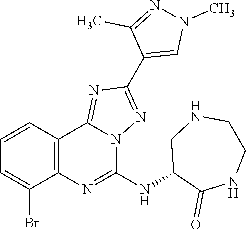 [SMILES: Cc1nn(C)cc1-c1nc2c3cccc(Br)c3nc(N[C@@H]3CNCCNC3=O)n2n1] |
| | (6R)-6-{[7-bromo-2-(1,3-dimethyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.93 min; MS (ESIpos): m/z = 470 [M + H]⁺ |
| | ¹H-NMR (400 MHz, DMSO-d₆): δ [ppm] = 2.60 (s, 3H), 3.03 (br t, 1H), 3.37- |
| | 3.47 (m, 3H), 3.67-3.77 (m, 1H), 3.85-3.89 (m, 3H), 3.93 (br d, 1H), 5.21 |
| | (br dd, 1H), 7.37 (t, 1H), 8.08 (dd, 1H), 8.19 (d, 1H), 8.26 (dd, 1H), 8.37 (s, |
| | 1H), 8.50 (br dd, 1H), 8.68-9.20 (m, 1H). |
| Example 963 | 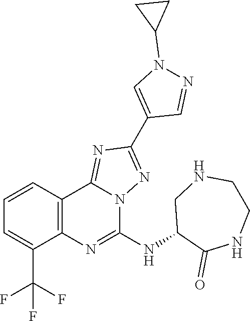 [SMILES: O=C1NCCNC[C@H]1Nc1nc2c(C(F)(F)F)cccc2c2nc(-c3cnn(C4CC4)c3)nn12] |
| | (6R)-6-{[2-(1-cyclopropyl-1H-pyrazol-4-yl)-7- |
| | (trifluoromethyl)[1,2,4]triazolo[1,5-c]quinazolin-5-yl]amino}-1,4- |
| | diazepan-5-one |
| | LC-MS (Method 2): Rₜ = 1.04 min; MS (ESIpos): m/z = 472 [M + H]⁺ |
| | ¹H-NMR (400 MHz, DMSO-d₆): δ [ppm] = 0.98-1.07 (m, 2H), 1.14-1.22 (m, |
| | 2H), 2.66-2.74 (m, 1H), 2.95-3.22 (m, 2H), 3.36-3.41 (m, 1H), 3.48 (dd, |
| | 1H), 3.89 (tt, 1H), 4.82 (br d, 1H), 7.54 (t, 1H), 7.82 (br d, 1H), 8.03-8.13 |
| | (m, 2H), 8.29 (dd, 1H), 8.49 (dd, 1H), 8.56 (s, 1H). |
| Example 964 | 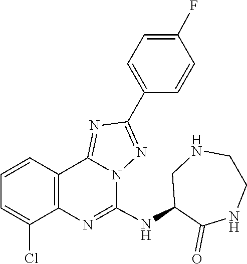 [SMILES: O=C1NCCNC[C@@H]1Nc1nc2c(Cl)cccc2c2nc(-c3ccc(F)cc3)nn12] |
| | (6S)-6-{[7-chloro-2-(4-fluorophenyl)[1,2,4]triazolo[1,5-c]quinazolin-5- |
| | yl]amino}-1,4-diazepan-5-one |
| | LC-MS (Method 2): Rt = 1.18 min; MS (ESIpos): m/z = 426 [M + H]⁺ |
| | ¹H-NMR (400 MHz, DMSO-d₆): δ [ppm] = 2.75 (dd, 1H), 3.06 (br dd, 1H), 3.10- |
| | 3.19 (m, 1H), 3.43 (br s, 2H), 3.54 (br dd, 1H), 4.92 (br dd, 1H), 7.39-7.48 |
| | (m, 3H), 7.89-7.95 (m, 2H), 8.27 (dd, 1H), 8.29-8.38 (m, 3H). |
| Example 965 | 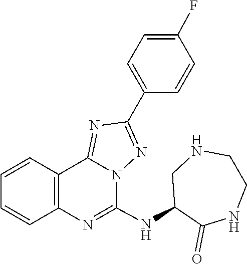 [SMILES: O=C1NCCNC[C@@H]1Nc1nc2ccccc2c2nc(-c3ccc(F)cc3)nn12] |
| | (6S)-6-{[2-(4-fluorophenyl)[1,2,4]triazolo[1,5-c]quinazolin-5-yl]amino}- |
| | 1,4-diazepan-5-one |
| | LC-MS (Method 2): Rₜ = 1.07 min; MS (ESIpos): m/z = 392 [M + H]⁺ |
| | ¹H-NMR (400 MHz, DMSO-d₆): δ [ppm] = 3.00 (br s, 1H), 3.20-3.29 (m, 2H), |
| | 3.42-3.63 (m, 1H), 3.71 (br d, 2H), 5.21 (br dd, 1H), 7.41-7.55 (m, 3H), |
| | 7.69-7.76 (m, 1H), 7.76-7.83 (m, 1H), 7.93 (d, 1H), 8.30-8.39 (m, 3H), |
| | 8.54 (br s, 1H), 8.86 (br s, 1H). |
| Example 966 | 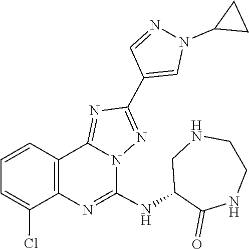 [SMILES: O=C1NCCNC[C@H]1Nc1nc2c(Cl)cccc2c2nc(-c3cnn(C4CC4)c3)nn12] |
| | (6R)-6-{[7-chloro-2-(1-cyclopropyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.99 min; MS (ESIpos): m/z = 438 [M + H]⁺ |
| | −> ¹H NMR (400 MHz, DMSO-d₆) δ ppm 0.98-1.06 (m, 2 H) 1.14-1.21 (m, |
| | 2 H) 2.65-2.76 (m, 1 H) 3.00-3.08 (m, 1 H) 3.08-3.18 (m, 1 H) 3.37-3.47 |
| | (m, 1 H) 3.48-3.55 (m, 1 H) 3.55-3.58 (m, 1 H) 3.79-3.99 (m, 1 H) 4.81- |
| | 4.95 (m, 1 H) 7.36-7.46 (m, 1 H) 7.70-7.79 (m, 1 H) 7.85-7.94 (m, 1 H) |
| | 8.02-8.11 (m, 1 H) 8.15-8.25 (m, 1 H) 8.26-8.35 (m, 1 H) 8.50-8.59 (m, |
| | 1 H) |
| Example 967 | 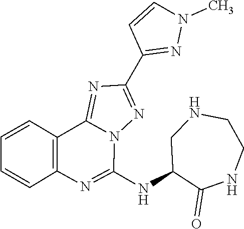 [SMILES: Cn1ccc(-c2nc3c4ccccc4nc(N[C@H]4CNCCNC4=O)n3n2)n1] |
| | (6S)-6-{[2-(1-methyl-1H-pyrazol-4-yl)[1,2,4]triazolo[1,5-c]quinazolin-5- |
| | yl]amino}-1,4-diazepan-5-one |
| | LC-MS (Method 2): Rₜ = 0.76 min; MS (ESIpos): m/z = 378 [M + H]⁺ |
| | ¹H-NMR (400 MHz, DMSO-d₆): δ [ppm] = 2.56 (br s, 1H), 2.68 (br dd, 1H), |
| | 3.04 (br dd, 1H), 3.08-3.38-3.48 (m, 2H), 3.99 (s, 3H), 4.82-4.92 (m, |
| | 1H), 6.92 (d, 1H), 7.46 (t, 1H), 7.62-7.79 (m, 3H), 7.90 (d, 1H), 8.16 (s, 1H), |
| | 8.26-8.36 (m, 2H). |
| Example 968 | 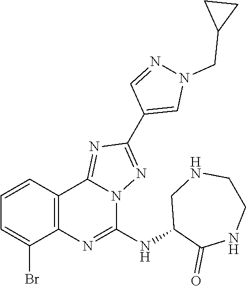 [SMILES: O=C1NCCNC[C@H]1Nc1nc2c(Br)cccc2c2nc(-c3cnn(CC4CC4)c3)nn12] |
| | (6R)-6-({7-bromo-2-[1-(cyclopropylmethyl)-1H-pyrazol-4- |
| | yl][1,2,4]triazolo[1,5-c]quinazolin-5-yl}amino)-1,4-diazepan-5-one |
| | LC-MS (Method 2): Rₜ = 1.04 min; MS (ESIpos): m/z = 496 [M + H]⁺ |
| | ¹H-NMR (400 MHz, DMSO-d₆): δ [ppm] = 0.41-0.47 (m, 2H), 0.54-0.61 (m, |
| | 2H), 1.28-1.38 (m, 1H), 2.55 (br d, 1H), 2.67-2.75 (m, 1H), 3.04 (dd, 1H), |
| | 3.08-3.17 (m, 1H), 3.38-3.47 (m, 1H), 3.54 (dd, 1H), 4.09 (d, 2H), 4.89 (td, |
| | 1H), 7.34 (t, 1H), 7.74 (d, 1H), 8.07 (dd, 1H), 8.10 (s, 1H), 8.26 (dd, 1H), |
| | 8.30 (dd, 1H), 8.55 (s, 1H). |
| Example 969 | 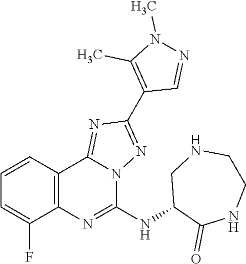 [SMILES: Cc1c(-c2nc3c4cccc(F)c4nc(N[C@@H]4CNCCNC4=O)n3n2)cnn1C] |
| | (6R)-6-{[2-(1,5-dimethyl-1H-pyrazol-4-yl)-7-fluoro[1,2,4]triazolo[1,5- |
| | c]quinazolin-5-yl]amino}-1,4-diazepan-5-one |
| | LC-MS (Method 2): Rₜ = 0.87 min; MS (ESIneg): m/z = 408 [M − H]⁻ |
| | ¹H-NMR (400 MHz, DMSO-d₆): δ [ppm] = 2.63-2.73 (m, 2H), 2.76 (s, 3H), |
| | 2.96-3.22 (m, 2H), 3.37-3.49 (m, 2H), 3.85 (s, 3H), 4.88 (td, 1H), 7.40 (td, |
| | 1H), 7.54-7.65 (m, 1H), 7.80 (br d, 1H), 7.99 (s, 1H), 8.07 (d, 1H), 8.27 (br |
| | dd, 1H). |
| Example 970 | 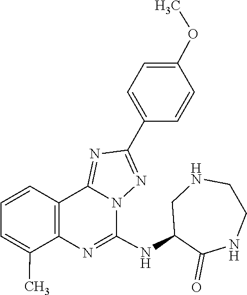 [SMILES: COc1ccc(-c2nc3c4cccc(C)c4nc(N[C@H]4CNCCNC4=O)n3n2)cc1] |
| | (6S)-6-{[2-(4-methoxyphenyl)-7-methyl[1,2,4]triazolo[1,5-c]quinazolin-5- |
| | yl]amino}-1,4-diazepan-5-one |
| | LC-MS (Method 2): Rₜ = 1.14 min; MS (ESIpos): m/z = 418 [M + H]⁺ |
| | ¹H-NMR (400 MHz, DMSO-d₆): δ [ppm] = 2.64 (s, 3H), 2.75-2.86 (m, 1H), |
| | 3.02 (br t, 1H), 3.19-3.29 (m, 2H), 3.52-3.62 (m, 1H), 3.69 (br d, 1H), 3.86 |
| | (s, 3H), 5.02-5.10 (m, 1H), 7.15 (d, 2H), 7.37 (t, 1H), 7.64 (d, 1H), 7.81 (br |
| | d, 1H), 8.15 (d, 1H), 8.23 (d, 2H), 8.42 (br s, 1H). |
| Example 971 | 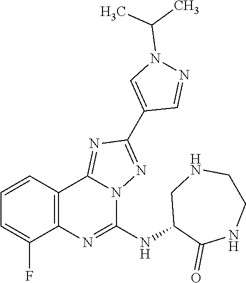 [SMILES: CC(C)n1cc(-c2nc3c4cccc(F)c4nc(N[C@@H]4CNCCNC4=O)n3n2)cn1] |
| | (6R)-6-({7-fluoro-2-[1-(propan-2-yl)-1H-pyrazol-4-yl][1,2,4]triazolo[1,5- |
| | c]quinazolin-5-yl}amino)-1,4-diazepan-5-one |
| | LC-MS (Method 2): Rₜ = 0.94 min; MS (ESIpos): m/z = 424 [M + H]⁺ |
| | ¹H-NMR (400 MHz, DMSO-d₆): δ [ppm] = 1.50 (d, 6H), 2.62-2.72 (m, 1H), |
| | 2.95-3.20 (m, 2H), 3.37-3.50 (m, 2H), 4.57-4.72 (m, 1H), 4.88 (ddd, 1H), |
| | 7.41 (td, 1H), 7.60 (ddd, 1H), 7.73 (d, 1H), 8.00-8.13 (m, 2H), 8.30 (dd, |
| | 1H), 8.53 (s, 1H). |
| Example 972 | 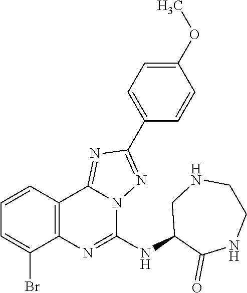 [SMILES: COc1ccc(-c2nc3c4cccc(Br)c4nc(N[C@H]4CNCCNC4=O)n3n2)cc1] |
| | (6S)-6-{[7-bromo-2-(4-methoxyphenyl)[1,2,4]triazolo[1,5-c]quinazolin-5- |
| | yl]amino}-1,4-diazepan-5-one |
| | LC-MS (Method 2): Rₜ = 1.20 min; MS (ESIpos): m/z = 484 [M + H]⁺ |
| | ¹H-NMR (400 MHz, DMSO-d₆): δ [ppm] = 2.53-2.60 (m, 1H), 2.72-2.81 (m, |
| | 1H), 2.98-3.11 (m, 1H), 3.11-3.21 (m, 1H), 3.39-3.52 (m, 1H), 3.56 (dd, |
| | 1H), 3.86 (s, 3H), 4.92 (br dd, 1H), 7.08-7.22 (m, 2H), 7.35 (t, 1H), 7.88 (d, |
| | 1H), 8.07 (dd, 1H), 8.22 (d, 2H), 8.30 (dd, 2H). |
| Example 973 | 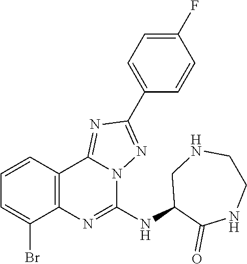 [SMILES: O=C1NCCNC[C@@H]1Nc1nc2c(Br)cccc2c2nc(-c3ccc(F)cc3)nn12] |
| | (6S)-6-{[7-bromo-2-(4-fluorophenyl)[1,2,4]triazolo[1,5-c]quinazolin-5- |
| | yl]amino}-1,4-diazepan-5-one |
| | LC-MS (Method 2): Rₜ = 1.24 min; MS (ESIpos): m/z = 472 [M + H]⁺ |
| | ¹H-NMR (400 MHz, DMSO-d₆): δ [ppm] = 2.52-2.58 (m, 1H), 2.69-2.80 (m, |
| | 1H), 3.05 (br dd, 1H), 3.09-3.19 (m, 1H), 3.38-3.48 (m, 1H), 3.55 (br d, |
| | 1H), 4.88-4.94 (m, 1H), 7.36 (t, 1H), 7.44 (t, 2H), 7.89 (d, 1H), 8.09 (dd, |
| | 1H), 8.27-8.37 (m, 4H). |
| Example 974 | 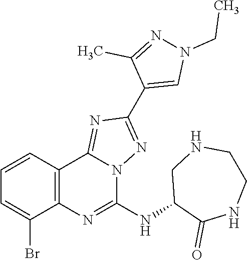 [SMILES: CCn1cc(-c2nc3c4cccc(Br)c4nc(N[C@@H]4CNCCNC4=O)n3n2)c(C)n1] |
| | (6R)-6-{[7-bromo-2-(1-ethyl-3-methyl-1H-pyrazol-4-yl[1,2,4]triazolo[1,5- |
| | c]quinazolin-5-yl]amino}-1,4-diazepan-5-one |
| | LC-MS (Method 2): Rₜ = 1.02 min; MS (ESIpos): m/z = 484 [M + H]⁺ |
| | ¹H-NMR (400 MHz, DMSO-d₆): δ [ppm] = 1.42 (t, 3H), 2.53 (br s, 1H), 2.59 (s, |
| | 3H), 2.68-2.78 (m, 1H), 2.99-3.11 (m, 1H), 3.11-3.20 (m, 1H), 3.44-3.50 |
| | (m, 1H), 3.58 (dd, 1H), 4.17 (q, 2H), 4.87-4.93 (m, 1H), 7.34 (t, 1H), 7.78 |
| | (d, 1H), 8.06 (dd, 1H), 8.24 (dd, 1H), 8.30 (dd, 1H), 8.41 (s, 1H). |
| Example 975 | 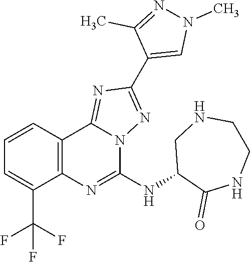 [SMILES: Cc1nn(C)cc1-c1nc2c3cccc(C(F)(F)F)c3nc(N[C@@H]3CNCCNC3=O)n2n1] |
| | (6R)-6-{[2-(1,3-dimethyl-1H-pyrazol-4-yl)-7- |
| | (trifluoromethyl)[1,2,4]triazolo[1,5-c]quinazolin-5-yl]amino}-1,4- |
| | diazepan-5-one |
| | LC-MS (Method 2): Rₜ = 0.98 min; MS (ESIpos): m/z = 460 [M + H]⁺ |
| | ¹H-NMR (400 MHz, DMSO-d₆): δ [ppm] = 2.58 (s, 3H), 2.63-2.74 (m, 1H), |
| | 3.01-3.16 (m, 2H), 3.36-3.42 (m, 1H), 3.45-3.55 (m, 1H), 3.87 (s, 3H), |
| | 4.77-4.84 (m, 1H), 7.54 (s, 1H), 7.85 (d, 1H), 8.08 (s, 1H), 8.28 (dd, 1H), |
| | 8.38 (s, 1H), 8.48 (dd, 1H). |
| Example 976 | 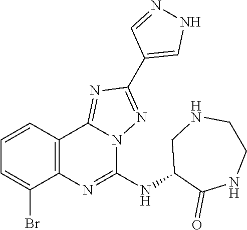 [SMILES: O=C1NCCNC[C@H]1Nc1nc2c(Br)cccc2c2nc(-c3cn[nH]c3)nn12] |
| | (6R)-6-{[7-bromo-2-(1H-pyrazol-4-yl)[1,2,4]triazolo[1,5-c]quinazolin-5- |
| | yl]amino}-1,4-diazepan-5-one |
| | LC-MS (Method 2): Rₜ = 0.82 min; MS (ESIpos): m/z = 442 [M + H]⁺ |
| | ¹H-NMR (400 MHz, DMSO-d₆): δ [ppm] = 2.52-2.57 (m, 1H), 2.68-2.74 (m, |
| | 1H), 3.04 (dd, 1H), 3.08-3.18 (m, 1H), 3.42 (br s, 1H), 3.53 (dd, 1H), 4.87- |
| | 4.93 (m, 1H), 7.34 (t, 1H), 7.74 (d, 1H), 8.07 (dd, 1H), 8.17 (s, 1H), 8.22- |
| | 8.36 (m, 2H), 8.36-8.59 (m, 1H), 13.38 (br s, 1H). |
| Example 977 | 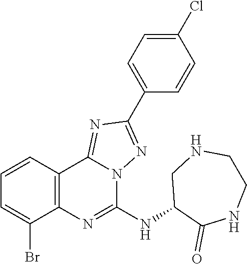 [SMILES: O=C1NCCNC[C@H]1Nc1nc2c(Br)cccc2c2nc(-c3ccc(Cl)cc3)nn12] |
| | (6R)-6-{[7-bromo-2-(4-chlorophenyl)[1,2,4]triazolo[1,5-c]quinazolin-5- |
| | yl]amino}-1,4-diazepan-5-one |
| | LC-MS (Method 2): Rₜ = 1.33 min; MS (ESIpos): m/z = 486 [M + H]⁺ |
| | ¹H-NMR (400 MHz, DMSO-d₆): δ [ppm] = 2.72 (br dd, 1H), 3.04 (dd, 1H), 3.13 |
| | (ddd, 1H), 3.38-3.49 (m, 1H), 3.54 (br d, 1H), 4.90 (br dd, 1H), 7.36 (t, 1H), |
| | 7.61-7.71 (m, 2H), 7.89 (d, 1H), 8.08 (dd, 1H), 8.25-8.35 (m, 4H). |
| Example 978 | 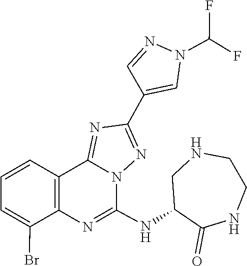 [SMILES: O=C1NCCNC[C@H]1Nc1nc2c(Br)cccc2c2nc(-c3cnn(C(F)F)c3)nn12] |
| | (6R)-6-({7-bromo-2-[1-(difluoromethyl)-1H-pyrazol-4- |
| | yl][1,2,4]triazolo[1,5-c]quinazolin-5-yl}amino)-1,4-diazepan-5-one |
| | LC-MS (Method 2): Rₜ = 1.01 min; MS (ESIpos): m/z = 492 [M + H]⁺ |
| | ¹H-NMR (400 MHz, DMSO-d₆): δ [ppm] = 2.52-2.56 (m, 2H), 2.65-2.76 (m, |
| | 1H), 3.05 (br dd, 1H), 3.09-3.19 (m, 1H), 3.54 (dd, 2H), 4.90 (br dd, 1H), |
| | 7.36 (t, 1H), 7.76-7.81 (m, 1H), 7.93 (s, 1H), 8.09 (dd, 1H), 8.17 (s, 1H), |
| | 8.27 (dd, 1H), 8.31 (dd, 1H), 8.45 (s, 1H), 9.02 (s, 1H). |
| Example 979 | 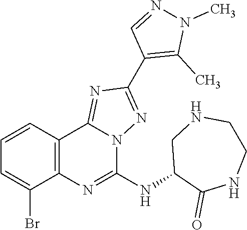 [SMILES: Cc1c(-c2nc3c4cccc(Br)c4nc(N[C@@H]4CNCCNC4=O)n3n2)cnn1C] |
| | (6R)-6-{[7-bromo-2-(1,5-dimethyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.95 min; MS (ESIpos): m/z = 470 [M + H]⁺ |
| | ¹H-NMR (400 MHz, DMSO-d₆): δ [ppm] = 2.52-2.57 (m, 1H), 2.69-2.75 (m, |
| | 1H), 2.76 (s, 3H), 3.00-3.08 (m, 1H), 3.08-3.18 (m, 1H), 3.41-3.46 (m, |
| | 1H), 3.53 (dd, 1H), 3.85 (s, 3H), 4.87-4.93 (m, 1H), 7.33 (t, 1H), 7.80 (d, |
| | 1H), 7.99 (s, 1H), 8.06 (dd, 1H), 8.27 (dd, 2H). |
| Example 980 | 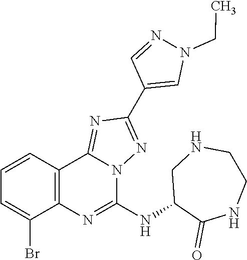 [SMILES: CCn1cc(-c2nc3c4cccc(Br)c4nc(N[C@@H]4CNCCNC4=O)n3n2)cn1] |
| | (6R)-6-{[7-bromo-2-(1-ethyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.95 min; MS (ESIpos): m/z = 470 [M + H]⁺ |
| | ¹H-NMR (400 MHz, DMSO-d₆): δ [ppm] = 1.45 (t, 3H), 2.52-2.57 (m, 1H), |
| | 2.65-2.74 (m, 1H), 3.04 (dd, 1H), 3.08-3.17 (m, 1H), 3.42 (br s, 1H), 3.54 |
| | (dd, 1H), 4.26 (q, 2H), 4.89 (ddd, 1H), 7.34 (t, 1H), 7.73 (d, 1H), 8.07 (dd, |
| | 1H), 8.09 (d, 1H), 8.17 (s, 1H), 8.25 (dd, 1H), 8.30 (dd, 1H), 8.53 (s, 1H). |
| Example 981 | 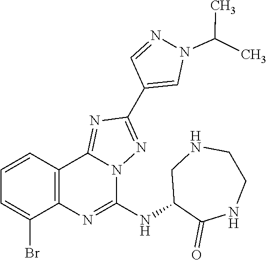 [SMILES: CC(C)n1cc(-c2nc3c4cccc(Br)c4nc(N[C@@H]4CNCCNC4=O)n3n2)cn1] |
| | (6R)-6-({7-bromo-2-[1-(propan-2-yl)-1H-pyrazol-4-yl][1,2,4]triazolo[1,5- |
| | c]quinazolin-5-yl}amino)-1,4-diazepan-5-one |
| | LC-MS (Method 2): Rₜ = 1.02 min; MS (ESIpos): m/z = 484 [M + H]⁺ |
| | ¹H-NMR (400 MHz, DMSO-d₆): δ [ppm] = 1.49 (d, 6H), 2.61-2.92 (m, 1H), |
| | 2.66-2.74 (m, 1H), 3.00-3.18 (m, 2H), 3.38-3.46 (m, 2H), 3.53 (dd, 1H), |
| | 4.64 (spt, 1H), 4.89 (br dd, 1H), 7.34 (t, 1H), 7.72 (d, 1H), 8.00-8.14 (m, |
| | 2H), 8.20-8.34 (m, 2H), 8.53 (s, 1H). |
| Example 982 | 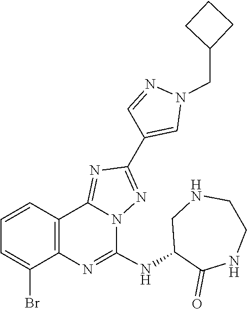 [SMILES: O=C1NCCNC[C@H]1Nc1nc2c(Br)cccc2c2nc(-c3cnn(CC4CCC4)c3)nn12] |
| | (6R)-6-({7-bromo-2-[1-(cyclobutylmethyl)-1H-pyrazol-4- |
| | yl][1,2,4]triazolo[1,5-c]quinazolin-5-yl}amino)-1,4-diazepan-5-one |
| | LC-MS (Method 2): Rₜ = 1.14 min; MS (ESIpos): m/z = 510 [M + H]⁺ |
| | ¹H-NMR (400 MHz, DMSO-d₆): δ [ppm] = 1.76-1.91 (m, 4H), 1.94-2.05 (m, |
| | 2H), 2.53-2.56 (m, 1H), 2.65-2.74 (m, 2H), 2.76-2.88 (m, 1H), 3.04 (br |
| | dd, 1H), 3.08-3.17 (m, 1H), 3.44 (br s, 1H), 3.53 (br dd, 1H), 4.25 (d, 2H), |
| | 4.89 (br dd, 1H), 7.34 (t, 1H), 7.73 (d, 1H), 8.08 (s, 2H), 8.25 (dd, 1H), 8.31 |
| | (br dd, 1H), 8.51 (s, 1H). |
| Example 983 | 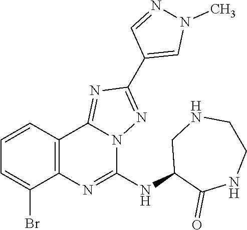 [SMILES: Cn1cc(-c2nc3c4cccc(Br)c4nc(N[C@H]4CNCCNC4=O)n3n2)cn1] |
| | (6S)-6-{[7-bromo-2-(1-methyl-1H-pyrazol-4-yl)[1,2,4]triazolo[1,5- |
| | c]quinazolin-5-yl]amino}-1,4-diazepan-5-one |
| | LC-MS (Method 2): Rₜ = 0.90 min; MS (ESIpos): m/z = 458 [M + H]⁺ |
| | ¹H-NMR (400 MHz, DMSO-d₆): δ [ppm] = 2.52-2.57 (m, 1H), 2.66-2.76 (m, |
| | 1H), 2.97-3.08 (m, 1H), 3.08-3.18 (m, 1H), 3.40 (br d, 1H), 3.54 (br d, 1H), |
| | 3.96 (s, 3H), 4.89 (br dd, 1H), 7.34 (t, 1H), 7.73 (d, 1H), 8.03-8.10 (m, 2H), |
| | 8.25 (dd, 1H), 8.31 (dd, 1H), 8.49 (s, 1H). |
| Example 984 | 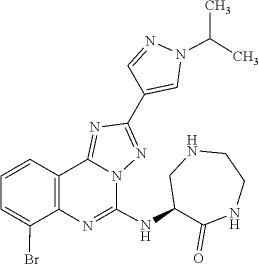 [SMILES: CC(C)n1cc(-c2nc3c4cccc(Br)c4nc(N[C@H]4CNCCNC4=O)n3n2)cn1] |
| | (6S)-6-({7-bromo-2-[1-(propan-2-yl)-1H-pyrazol-4-yl][1,2,4]triazolo[1,5- |
| | c]quinazolin-5-yl}amino)-1,4-diazepan-5-one |
| | LC-MS (Method 2): Rt = 1.05 min; MS (ESIpos): m/z = 484 [M + H]⁺ |
| | ¹H-NMR (400 MHz, DMSO-d₆): δ [ppm] = 1.49 (d, 6H), 2.63-2.91 (m, 2H), |
| | 3.04 (br dd, 1H), 3.07-3.17 (m, 1H), 3.37-3.47 (m, 1H), 3.49-3.57 (m, |
| | 1H), 4.58-4.69 (m, 1H), 4.85-4.93 (m, 1H), 7.34 (t, 1H), 7.72 (br d, 1H), |
| | 8.06 (dd, 1H), 8.09 (s, 1H), 8.25 (dd, 1H), 8.30 (dd, 1H), 8.53 (s, 1H). |

### Example 985

### (2R)-2-{[7-bromo-2-(1-methyl-1H-pyrazol-4-yl)[1,2,4]triazolo[1,5-c]quinazolin-5-yl]amino}-1-(piperazin-1-yl)propan-1-one hydrogen chloride (1/1)

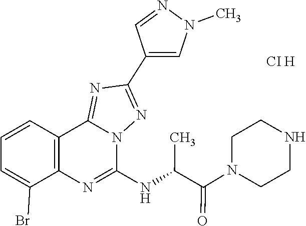 [SMILES: C[C@@H](Nc1nc2c(Br)cccc2c2nc(-c3cnn(C)c3)nn12)C(=O)N1CCNCC1 ; "CI H"]

Hydrochloric acid (250 μL, 4.0 M, 1.0 mmol) in 1,4-dioxane was slowly added dropwise to tert-butyl 4-{N-[7-bromo-2-(1-methyl-1H-pyrazol-4-yl)[1,2,4]triazolo[1,5-c]quinazolin-5-yl]-D-alanyl}piperazine-1-carboxylate (59.0 mg, 101 μmol) in 1,4-dioxane (0.70 mL). It was stirred for 3 h at rt. Then additional hydrochloric acid (200 μL, 4.0 M, 800 μmol) in 1,4-dioxane (0.20 mL) was added and stirred at rt overnight. The precipitate was filtered and washed four times with MTBE (1 mL). The residue was dried under vacuum at 60° C. to obtain 51 mg (97%) of the title compound.

LC-MS (Method 2): Rₜ=0.96 md; MS (ESIpos): m/z=484 [M+H]⁺

1H-NMR (400 MHz, DMSO-d₆): δ [ppm]=1.55 (d, 3H), 3.06-3.29 (m, 4H), 3.59-3.69 (m, 1H), 3.77-3.87 (m, 1H), 3.92-4.02 (m, 5H), 5.16 (quin, 1H), 7.33 (t, 1H), 8.04-8.09 (m, 2H), 8.25 (dd, 1H), 8.29 (d, 1H), 8.45 (s, 1H), 9.11 (br s, 2H).

The following examples were prepared analogously to example 985.

| | Structure |
|---|---|
| | IUPAC-Name |
| | LC-MS (method): Retention time; Mass found |
| Example | ¹H-NMR |
| Example 986 | 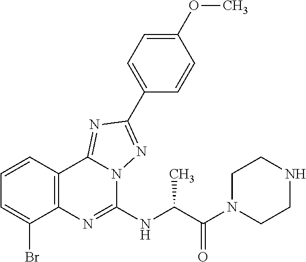 [SMILES: COc1ccc(-c2nc3c4cccc(Br)c4nc(N[C@H](C)C(=O)N4CCNCC4)n3n2)cc1] |
| | (2R)-2-{[7-bromo-2-(4-methoxyphenyl)[1,2,4]triazolo[1,5-c]quinazolin-5- |
| | yl]amino}-1-(piperazin-1-yl)propan-1-one |
| | LC-MS (Method 2): Rt = 1.38 min; MS (ESIpos): m/z = 510 [M + H]⁺ |
| | ¹H-NMR (400 MHz, DMSO-d₆): δ [ppm] = 1.54 (d, 3H), 2.64-2.82 (m, 3H), 2.85- |
| | 2.93 (m, 1H), 3.48-3.57 (m, 2H), 3.60-3.67 (m, 2H), 3.86 (s, 3H), 5.16- |
| | 5.25 (m, 1H), 7.16 (d, 2H), 7.33 (t, 1H), 8.06 (dd, 1H), 8.21 (br d, 1H), 8.26 (d, |
| | 2H), 8.30 (dd, 1H). |
| Example 987 | 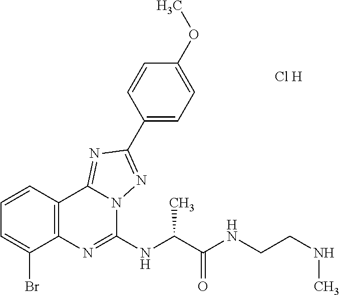 [SMILES: CNCCNC(=O)[C@@H](C)Nc1nc2c(Br)cccc2c2nc(-c3ccc(OC)cc3)nn12 ; "Cl H"] |
| | N²-[7-bromo-2-(4-methoxyphenyl)[1,2,4]triazolo[1,5-c]quinazolin-5-yl]-N- |
| | [2-(methylamino)ethyl]-D-alaninamide-hydrogen chloride (1/1) |
| | LC-MS (Method 2): Rt = 1.25 min; MS (ESIpos): m/z = 498 [M + H]⁺ |
| | ¹H-NMR (400 MHz, DMSO-d₆): δ [ppm] = 1.61 (d, 3H), 2.89-3.05 (m, 2H), 3.32- |
| | 3.41 (m, 1H), 3.42-3.52 (m, 1H), 3.86 (s, 3H), 4.82 (quin, 1H), 7.16 (d, 2H), |
| | 7.34 (t, 1H), 8.03-8.08 (m, 1H), 8.22-8.28 (m, 3H), 8.28-8.33 (m, 1H), 8.44 |
| | (br t, 1H), 8.78 (br d, 2H). |
| Example 988 | 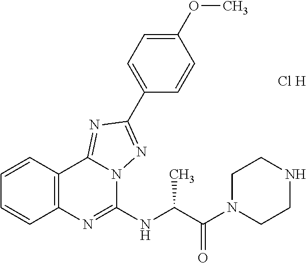 [SMILES: COc1ccc(-c2nc3c4ccccc4nc(N[C@H](C)C(=O)N4CCNCC4)n3n2)cc1 ; "Cl H"] |
| | (2R)-2-{[2-(4-methoxyphenyl)[1,2,4]triazolo[1,5-c]quinazolin-5-yl]amino}- |
| | 1-(piperazin-1-yl)propan-1-one-hydrogen chloride (1/1) |
| | LC-MS (Method 2): Rt = 1.15 min; MS (ESIpos): m/z = 432 [M + H]⁺ |
| | ¹H-NMR (400 MHz, DMSO-d₆): δ [ppm] = 1.52 (d, 3H), 2.97-3.08 (m, 1H), 3.12- |
| | 3.29 (m, 3H), 3.55-3.65 (m, 1H), 3.80-3.92 (m, 5H), 3.98-4.09 (m, 1H), |
| | 5.21 (quin, 1H), 7.15 (d, 2H), 7.43-7.49 (m, 1H), 7.64-7.69 (m, 1H), 7.70- |
| | 7.77 (m, 1H), 8.03 (d, 1H), 8.25 (d, 2H), 8.30 (dd, 1H), 9.23 (br d, 2H). |
| Example 989 | 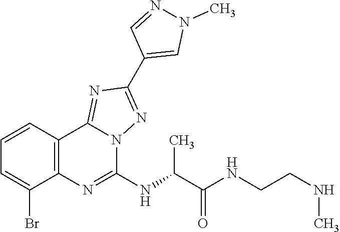 [SMILES: CNCCNC(=O)[C@@H](C)Nc1nc2c(Br)cccc2c2nc(-c3cnn(C)c3)nn12] |
| | N²-[7-bromo-2-(1-methyl-1H-pyrazol-4-yl)[1,2,4]triazolo[1,5-c]quinazolin- |
| | 5-yl]-N-[2-(methylamino)ethyl]-D-alaninamide |
| | LC-MS (Method 2): Rt = 1.09 min; MS (ESIpos): m/z = 472 [M + H]⁺ |
| | ¹H-NMR (400 MHz, DMSO-d₆): δ [ppm] = 1.56 (d, 3H), 2.25 (s, 3H), 2.57 (br t, |
| | 2H), 3.12-3.26 (m, 2H), 3.96 (s, 3H), 4.78 (q, 1H), 7.32 (t, 1H), 8.02-8.09 |
| | (m, 2H), 8.17 (br t, 1H), 8.24 (d, 1H), 8.46 (s, 1H). |
| Example 990 | 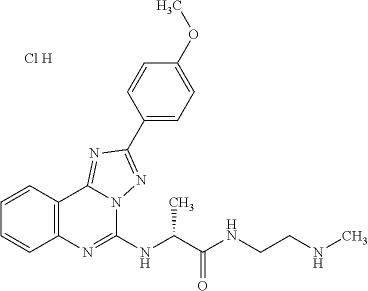 [SMILES: CNCCNC(=O)[C@@H](C)Nc1nc2ccccc2c2nc(-c3ccc(OC)cc3)nn12 ; "Cl H"] |
| | N²-[2-(4-methoxyphenyl)[1,2,4]triazolo[1,5-c]quinazolin-5-yl]-N-[2- |
| | (methylamino)ethyl]-D-alaninamide-hydroxy chloride (1/1) |
| | LC-MS (Method 2): Rt = 1.15 min; MS (ESIpos): m/z = 420 [M + H]⁺ |
| | ¹H-NMR (400 MHz, DMSO-d₆): δ [ppm] = 1.56 (d, 3H), 2.54 (t, 3H), 2.90-3.03 |
| | (m, 2H), 3.28-3.50 (m, 2H and water signal), 3.86 (s, 3H), 4.82 (quin, 1H), |
| | 7.13-7.18 (m, 2H), 7.45 (ddd, 1H), 7.64 (d, 1H), 7.73 (ddd, 1H), 7.90 (d, 1H), |
| | 8.23-8.28 (m, 2H), 8.30 (dd, 1H), 8.42 (t, 1H), 8.58 (br d, 2H). |
| Example 991 | 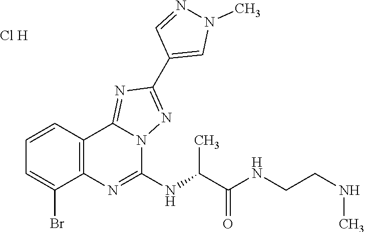 [SMILES: "Cl H" ; CNCCNC(=O)[C@@H](C)Nc1nc2c(Br)cccc2c2nc(-c3cnn(C)c3)nn12] |
| | N²-[7-bromo-2-(1-methyl-1H-pyrazol-4-yl)[1,2,4]triazolo[1,5-c]quinazolin- |
| | 5-yl]-N-[2-(methylamino)ethyl]-D-alaninamide-hydrogen chloride (1/1) |
| | LC-MS (Method 2): Rt = 0.97 min; MS (ESIpos): m/z = 472 [M + H]⁺ |
| | ¹H-NMR (400 MHz, DMSO-d₆): δ [ppm] = 1.59 (d, 3H), 2.52-2.56 (m, 3H), 2.90- |
| | 3.05 (m, 2H), 3.39-3.52 (m, 1H), 3.96 (s, 3H), 4.80 (quin, 1H), 7.33 (t, 1H), |
| | 8.03-8.08 (m, 2H), 8.11 (d, 1H), 8.25 (dd, 1H), 8.42 (t, 1H), 8.45 (s, 1H), 8.59 |
| | (br d, 2H). |
| Example 992 | 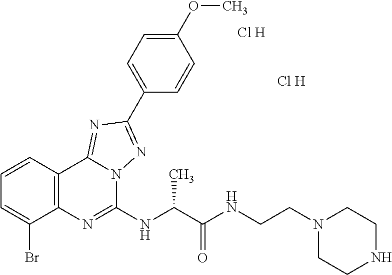 |
| | N²-[7-bromo-2-(4-methoxyphenyl)[1,2,4]triazolo[1,5-c]quinazolin-5-yl]-N- |
| | [2-(piperazin-1-yl)ethyl]-D-alaninamide-hydrogen chloride (1/2) |
| | LC-MS (Method 2): Rt = 1.19 min; MS (ESIpos): m/z = 553 [M + H]⁺ |
| | ¹H-NMR (400 MHz, DMSO-d₆): δ [ppm] = 1.62 (d, 3H), 3.10-3.31 (m, 4H), 3.64- |
| | 3.73 (m, 1H), 3.86 (s, 3H), 4.83 (quin, 1H), 7.14-7.18 (m, 2H), 7.34 (t, 1H), |
| | 8.06 (dd, 1H), 8.23-8.28 (m, 2H), 8.30 (dd, 1H), 8.36 (br d, 1H), 8.44 (br t, |
| | 1H), 9.48 (br s, 2H), 11.72 (br s, 1H). |
| Example 993 | 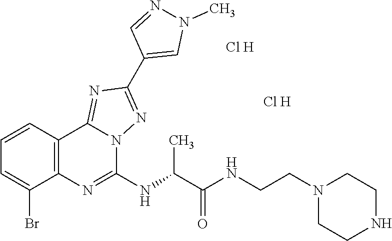 |
| | N²-[7-bromo-2-(1-methyl-1H-pyrazol-4-yl)[1,2,4]triazolo[1,5-c]quinazolin- |
| | 5-yl]-N-[2-(piperazin-1-yl)ethyl]-D-alaninamide-hydrogen chloride (1/2) |
| | LC-MS (Method 2): Rt = 0.92 min; MS (ESIpos): m/z = 527 [M + H]⁺ |
| | ¹H-NMR (400 MHz, DMSO-d₆): δ [ppm] = 1.60 (d, 3H), 3.03-3.30 (m, 4H), 3.48- |
| | 3.78 (m, 4H), 3.96 (s, 3H), 4.80 (quin, 1H), 7.33 (t, 1H), 8.05 (dd, 1H), 8.07 |
| | (s, 1H), 8.21 (br d, 1H), 8.25 (dd, 1H), 8.38-8.49 (m, 2H), 9.41 (br s, 2H), |
| | 11.69 (br s, 1H). |

The following examples were prepared following the same procedure as for example 656:

| | Structure |
|---|---|
| | IUPAC-Name |
| | LC-MS (method): Retention time; Mass found |
| Example | ¹H-NMR |
| Example 994 | 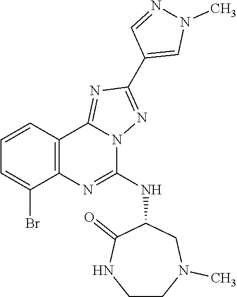 [SMILES: CN1CCNC(=O)[C@H](Nc2nc3c(Br)cccc3c3nc(-c4cnn(C)c4)nn23)C1] |
| | (6R)-6-{[7-bromo-2-(1-methyl-1H-pyrazol-4-yl)[1,2,4]triazolo[1,5- |
| | c]quinazolin-5-yl]amino}-1-methyl-1,4-diazepan-5-one |
| | LC-MS (Method 2): Rₜ = 1.02 min; MS (ESIpos): m/z = 470 [M + H]⁺ |
| | ¹H-NMR (400 MHz, DMSO-d₆): δ [ppm] = 2.21 (t, 1H), 2.31-2.38 (m, 1H), 2.47 |
| | (s, 3H), 2.90 (br dd, 1H), 3.06-3.14 (m, 1H), 3.44-3.57 (m, 1H), 3.57-3.61 |
| | (m, 1H), 3.95 (s, 3H), 4.99-5.05 (m, 1H), 7.34 (t, 1H), 7.78 (d, 1H), 8.05-8.09 |
| | (m, 2H), 8.25 (dd, 1H), 8.36 (dd, 1H), 8.49 (s, 1H). |
| Example 995 | 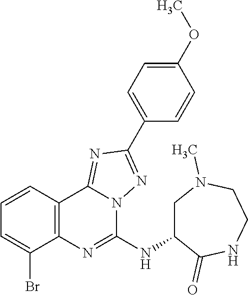 [SMILES: COc1ccc(-c2nc3c4cccc(Br)c4nc(N[C@@H]4CN(C)CCNC4=O)n3n2)cc1] |
| | (6R)-6-{[7-bromo-2-(4-methoxyphenyl)[1,2,4]triazolo[1,5-c]quinazolin-5- |
| | yl]amino}-1-methyl-1,4-diazepan-5-one |
| | LC-MS (Method 2): Rₜ = 1.29 min; MS (ESIpos): m/z = 496 [M + H]⁺ |
| | ¹H-NMR (400 MHz, DMSO-d₆): δ [ppm] = 2.22 (br t, 1H), 2.37 (dd, 1H), 2.48 (s, |
| | 3H), 2.91 (br dd, 1H), 3.07-3.15 (m, 1H), 3.45-3.57 (m, 2H), 3.86 (s, 3H), |
| | 5.04 (ddd, 1H), 7.12-7.17 (m, 2H), 7.35 (t, 1H), 7.90 (d, 1H), 8.08 (dd, 1H), |
| | 8.19-8.24 (m, 2H), 8.31 (dd, 1H), 8.36 (dd, 1H). |

### Example 996

### (3R)-3-{[7-(3-methoxy-3-methylbutoxy)-2-(4-methoxyphenyl)[1,2,4]triazolo[1,5-c]quinazolin-5-yl]amino}azepan-2-one

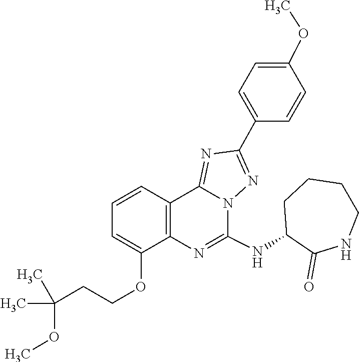 [SMILES: COc1ccc(-c2nc3c4cccc(OCCC(C)(C)OC)c4nc(N[C@@H]4CCCCNC4=O)n3n2)cc1]

The title compound was prepared similarly to example 667 starting from (3R)-3-{[7-bromo-2-(4-methoxyphenyl)[1,2,4]triazolo[1,5-c]quinazolin-5-yl]amino}azepan-2-one (50.0 mg, 104 μmol) in 3% yield (2 mg, 90% purity).

¹H-NMR (400 MHz, DMSO-d6) δ[ppm]: 0.828 (0.76), 0.851 (0.76), 1.107 (1.13), 1.139 (1.13), 1.232 (5.17), 1.240 (14.74), 1.267 (1.26), 1.319 (1.13), 1.352 (1.26), 1.421 (0.63), 1.537 (0.63), 1.570 (0.50), 1.855 (0.63), 1.887 (0.63), 1.902 (1.13), 2.026 (0.88), 2.043 (1.51), 2.057 (1.51), 2.073 (0.76), 2.332 (2.52), 2.336 (1.26), 2.518 (16.00), 2.522 (10.33), 2.673 (2.52), 3.139 (13.98), 3.257 (0.50), 3.283 (1.13), 3.858 (11.21), 4.206 (1.01), 4.222 (2.02), 4.239 (0.88), 7.130 (2.77), 7.152 (2.77), 7.301 (0.63), 7.317 (1.26), 7.321 (1.13), 7.341 (1.26), 7.361 (1.64), 7.381 (0.63), 7.631 (1.01), 7.645 (1.01), 7.849 (1.13), 7.853 (1.13), 7.869 (1.01), 7.872 (1.01), 8.208 (3.40), 8.214 (1.39), 8.231 (2.90).

### Example 997

### (3R)-3-{[7-(2-hydroxy-2-methylpropoxy)-2-(4-methoxyphenyl)[1,2,4]triazolo[1,5-c]quinazolin-5-yl]amino}azepan-2-one

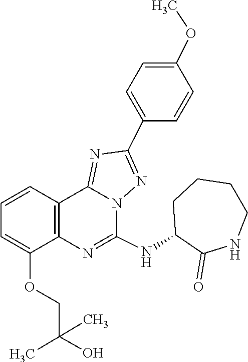 [SMILES: COc1ccc(-c2nc3c4cccc(OCC(C)(C)O)c4nc(N[C@@H]4CCCCNC4=O)n3n2)cc1]

The title compound was prepared similarly to example 667 starting from (3R)-3-{[7-bromo-2-(4-methoxyphenyl)[1,2,4]triazolo[1,5-c]quinazolin-5-yl]amino}azepan-2-one (50.0 mg, 104 μmol) in 2% yield (1 mg, 90% purity).

¹H-NMR (400 MHz, DMSO-d6) δ[ppm]: 0.834 (1.14), 0.851 (1.14), 0.967 (0.53), 1.108 (1.90), 1.144 (0.76), 1.232 (5.99), 1.256 (1.82), 1.332 (10.16), 1.370 (9.48), 1.422 (0.99), 1.520 (0.68), 1.552 (0.83), 1.580 (0.76), 1.842 (0.76), 1.876 (0.99), 1.905 (1.82), 1.994 (0.68), 2.074 (0.45), 2.085 (0.53), 2.113 (0.45), 2.176 (0.45), 2.318 (1.44), 2.323 (2.88), 2.327 (3.87), 2.331 (2.81), 2.337 (1.36), 2.387 (0.76), 2.518 (14.03), 2.523 (9.71), 2.660 (1.29), 2.665 (2.81), 2.669 (3.72), 2.673 (2.65), 3.247 (0.61), 3.860 (16.00), 3.880 (2.96), 3.885 (2.96), 4.697 (3.94), 4.831 (0.45), 4.847 (0.45), 4.858 (0.45), 4.873 (0.45), 7.133 (3.72), 7.155 (3.64), 7.261 (0.99), 7.278 (1.52), 7.280 (1.44), 7.338 (1.52), 7.357 (2.27), 7.377 (0.99), 7.690 (1.36), 7.703 (1.29), 7.846 (1.52), 7.849 (1.59), 7.866 (1.44), 7.869 (1.36), 8.193 (0.53), 8.212 (4.55), 8.218 (1.59), 8.229 (1.36), 8.235 (3.64).

### Example 998

### (3R)-3-{[7-(cyclobutyloxy)-2-(4-methoxyphenyl)[1,2,4]triazolo[1,5-c]quinazolin-5-yl]amino}azepan-2-one

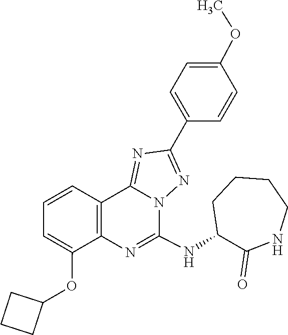 [SMILES: COc1ccc(-c2nc3c4cccc(OC5CCC5)c4nc(N[C@@H]4CCCCNC4=O)n3n2)cc1]

The title compound was prepared similarly to example 667 starting from (3R)-3-{[7-bromo-2-(4-methoxyphenyl)[1,2,4]triazolo[1,5-c]quinazolin-5-yl]amino}azepan-2-one (50.0 mg, 104 μmol) in 1% yield (1 mg, 90% purity).

¹H-NMR (400 MHz, DMSO-d6) δ[ppm]: 0.811 (1.00), 0.827 (1.25), 0.851 (1.25), 0.859 (1.00), 0.966 (1.00), 1.046 (0.50), 1.078 (0.50), 1.095 (0.75), 1.106 (2.00), 1.116 (1.00), 1.139 (2.00), 1.161 (0.75), 1.195 (1.00), 1.231 (7.50), 1.249 (2.25), 1.266 (2.00), 1.295 (1.25), 1.319 (1.50), 1.332 (1.50), 1.347 (1.75), 1.422 (0.75), 1.490 (0.75), 1.519 (0.75), 1.550 (0.75), 1.662 (0.50), 1.683 (0.75), 1.710 (0.75), 1.734 (0.50), 1.842 (1.00), 1.867 (1.25), 1.897 (1.00), 1.906 (1.25), 2.040 (0.50), 2.083 (0.50), 2.112 (0.50), 2.168 (1.00), 2.178 (1.00), 2.195 (0.75), 2.318 (1.50), 2.322 (3.00), 2.326 (4.25), 2.331 (3.00), 2.336 (1.50), 2.418 (1.00), 2.518 (16.00), 2.522 (10.50), 2.539 (1.00), 2.659 (1.50), 2.664 (3.00), 2.668 (4.00), 2.673 (3.00), 2.678 (1.25), 3.201 (0.50), 3.470 (0.75), 3.504 (0.75), 3.777 (0.75), 3.857 (15.25), 4.807 (0.50), 4.821 (0.50), 4.834 (0.50), 4.846 (0.50), 4.892 (0.75), 4.909 (1.00), 4.927 (0.75), 7.129 (4.75), 7.151 (5.00), 7.313 (1.50), 7.332 (2.25), 7.353 (1.25), 7.621 (1.50), 7.633 (1.25), 7.844 (1.50), 7.847 (1.75), 7.863 (1.50), 7.866 (1.50), 8.206 (4.50), 8.211 (2.00), 8.223 (1.50), 8.228 (3.75).

### Example 999

### (3R)-3-{[7-(cyclopropyloxy)-2-(4-methoxyphenyl)[1,2,4]triazolo[1,5-c]quinazolin-5-yl]amino}azepan-2-one

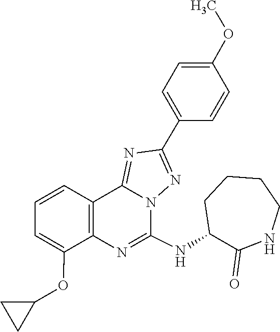 [SMILES: COc1ccc(-c2nc3c4cccc(OC5CC5)c4nc(N[C@@H]4CCCCNC4=O)n3n2)cc1]

The title compound has been was prepared similarly to example 667 starting from (3R)-3-{[7-bromo-2-(4-methoxyphenyl)[1,2,4]triazolo[1,5-c]quinazolin-5-yl]amino}azepan-2-one (50.0 mg, 104 μmol) in 6% yield (3 mg, 95% purity).

LC-MS (method 2): Rₜ=1.24 min; MS (ESIpos): m/z=459 [M+H]⁺

¹H-NMR (400 MHz, DMSO-d6): δ [ppm]=0.71-0.89 (m, 4H), 1.27-1.37 (m, 2H), 1.44-1.60 (m, 2H), 1.80-1.93 (m, 1H), 2.03 (br d, 1H), 2.35-2.41 (m, 1H), 3.11-3.22 (m, 1H), 3.86 (s, 3H), 4.12 (tt, 1H), 4.79 (br dd, 1H), 7.09-7.18 (m, 2H), 7.40 (t, 1H), 7.55 (dd, 1H), 7.63 (d, 1H), 7.90 (dd, 1H), 8.15-8.27 (m, 3H).

### Example 1000

### (3R)-3-{[7-(3-hydroxy-3-methylbutoxy)-2-(4-methoxyphenyl)[1,2,4]triazolo[1,5-c]quinazolin-5-yl]amino}azepan-2-one

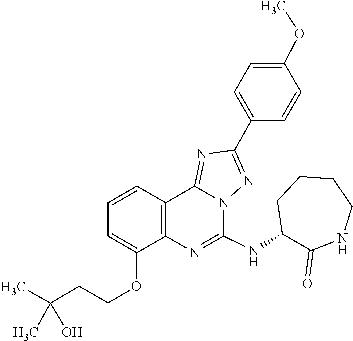 [SMILES: COc1ccc(-c2nc3c4cccc(OCCC(C)(C)O)c4nc(N[C@@H]4CCCCNC4=O)n3n2)cc1]

The title compound was prepared similarly to example 667 starting from (3R)-3-{[7-bromo-2-(4-methoxyphenyl)[1,2,4]triazolo[1,5-c]quinazolin-5-yl]amino}azepan-2-one (50.0 mg, 104 μmol) in 5% yield (3 mg, 90% purity).

¹H-NMR (400 MHz, DMSO-d6) δ[ppm]: 0.831 (1.18), 0.849 (1.31), 1.107 (1.00), 1.226 (16.00), 1.242 (13.01), 1.313 (1.63), 1.347 (1.59), 1.423 (0.73), 1.510 (0.77), 1.546 (1.04), 1.575 (0.86), 1.842 (0.82), 1.879 (0.77), 1.905 (0.77), 1.921 (0.82), 1.936 (1.00), 1.955 (1.54), 1.971 (2.08), 1.986 (2.40), 2.001 (2.04), 2.019 (1.27), 2.036 (0.77), 2.054 (0.45), 2.084 (1.63), 2.302 (0.95), 2.327 (2.18), 2.669 (1.59), 3.159 (0.54), 3.194 (0.68), 3.504 (0.41), 3.858 (13.37), 4.274 (0.77), 4.290 (1.72), 4.303 (1.68), 4.319 (0.77), 4.553 (3.90), 4.836 (0.63), 4.849 (0.73), 4.862 (0.68), 4.876 (0.63), 7.129 (3.40), 7.152 (3.58), 7.305 (1.00), 7.323 (1.86), 7.352 (1.50), 7.371 (2.04), 7.390 (0.82), 7.620 (1.50), 7.634 (1.50), 7.851 (1.63), 7.870 (1.45), 8.184 (0.73), 8.210 (4.31), 8.232 (3.54).

### Example 1001

### (3R)-3-{[2-(4-methoxyphenyl)-7-(2-oxopyrrolidin-1-yl)[1,2,4]triazolo[1,5-c]quinazolin-5-yl]amino}azepan-2-one

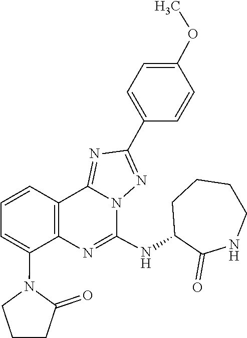 [SMILES: COc1ccc(-c2nc3c4cccc(N5CCCC5=O)c4nc(N[C@@H]4CCCCNC4=O)n3n2)cc1]

(3R)-3-{[7-Bromo-2-(4-methoxyphenyl)[1,2,4]triazolo[1,5-c]quinazolin-5-yl]amino}azepan-2-one (50.0 mg, 104 μmol), pyrrolidin-2-one (12 μL, 160 μmol) and cesium carbonate (67.7 mg, 208 μmol) were solubilised in 1,4-dioxane (450 μL), and the reaction mixture was sparged with argon. Pd₂(dba)₃ (6.66 mg, 7.27 μmol; CAS-RN:[51364-51-3]) and Xantphos (4.21 mg, 7.27 μmol) were added and the mixture was stirred for 2 h at 110° C. The reaction mixture was cooled to rt, concentrated under reduced pressure and the crude material was purified by preparative HPLC to give 29.2 mg (100% purity, 58% yield) of the title compound.

LC-MS (method 2): Rₜ=1.15 min; MS (ESIneg): m/z=484 [M−H]⁻.

¹H-NMR (400 MHz, DMSO-d6): δ [ppm]=1.26-1.39 (m, 1H), 1.52-1.65 (m, 1H), 1.73-1.91 (m, 2H), 1.99-2.10 (m, 1H), 2.23 (quin, 2H), 2.33 (td, 1H), 3.19 (br dd, 1H), 3.86 (s, 3H), 3.96-4.07 (m, 2H), 4.78 (br d, 1H), 7.15 (d, 2H), 7.46 (t, 1H), 7.67 (d, 1H), 7.88 (br d, 1H), 8.19-8.29 (m, 4H).

### Example 1002

### (3R)-3-{[2-(4-methoxyphenyl)-7-(oxetan-3-yl)[1,2,4]triazolo[1,5-c]quinazolin-5-yl]amino}azepan-2-one

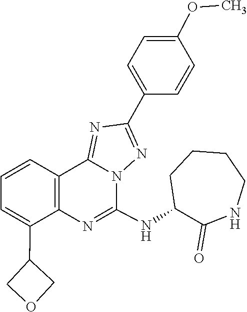 [SMILES: COc1ccc(-c2nc3c4cccc(C5COC5)c4nc(N[C@@H]4CCCCNC4=O)n3n2)cc1]

(3R)-3-{[7-Bromo-2-(4-methoxyphenyl)[1,2,4]triazolo[1,5-c]quinazolin-5-yl]amino}azepan-2-one (50.0 mg, 104 μmol), lithium carbonate (46.1 mg, 623 μmol) and Ir(4′,6′-dF-5-CF3-ppy)2(4,4′-dtbbpy)PF6 (2.33 mg, 2.08 μmol, CAS [870987-63-6]) were dissolved in the reaction vial in trifluorotoluene (3.0 mL). In a separate vial, nickel (II) chloride dimethoxyethane adduct (110 μg, 0.52 μmol) and 4,4′-di-tert-butyl-2,2′-bipyridine (140 μg, 0.52 μmol) were solubilised in N,N-dimethylacetamide (1 mL) and the mixture was stirred for 5 minutes. The catalyst solution was added to the sealed reaction vial. The reaction was sparged with argon in an ultrasonic bath for 5 minutes. 3-Bromooxetane (39 μL, 470 μmol) and tris(trimethylsilyl)silan (32 μL, 100 μmol) were then added. The reaction vessel was placed in a water bath and irradiated with two 40 W Kessil LED Aquarium lamps for 16 h. The reaction mixture was then diluted with ethyl acetate and washed with half saturated. aqueous sodium chloride solution. The organic phase was dried (silicon filter) and concentrated under reduced pressure. The crude mixture was purified by preparative HPLC to give 6.90 mg (92% purity, 13% yield) of the title compound.

LC-MS (method 2): Rₜ=1.27 min; MS (ESIpos): m/z=459 [M+H]⁺

¹H-NMR (400 MHz, DMSO-d6): δ [ppm]=1.27-1.40 (m, 1H), 1.51-1.64 (m, 1H), 1.84-2.00 (m, 2H), 2.02-2.12 (m, 1H), 2.28 (br d, 1H), 3.14-3.24 (m, 1H), 3.36-3.43 (m, 1H), 3.85 (s, 3H), 4.78 (dd, 1H), 4.83-4.96 (m, 3H), 5.04-5.15 (m, 2H), 7.11-7.16 (m, 2H), 7.44 (t, 1H), 7.68 (d, 1H), 7.75 (d, 1H), 8.16-8.26 (m, 4H).

### Example 1003

### (3R)-3-{[7-(methoxymethyl)-2-(4-methoxyphenyl)[1,2,4]triazolo[1,5-c]quinazolin-5-yl]amino}azepan-2-one

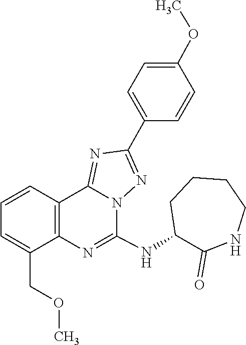 [SMILES: COCc1cccc2c1nc(N[C@@H]1CCCCNC1=O)n1nc(-c3ccc(OC)cc3)nc21]

(3R)-3-{[7-Bromo-2-(4-methoxyphenyl)[1,2,4]triazolo[1,5-c]quinazolin-5-yl]amino}azepan-2-one (100 mg, 208 μmol), potassium trifluorido(methoxymethyl)borate (63.1 mg, 415 μmol), [Ir(dF(CF3)ppy2)2(dtbbpy)]PF6 (4.66 mg, 4.15 μmol) and sodium carbonate (44.0 mg, 415 μmol) were dissolved in DMA (3.7 mL) in a microwave vial. A stock solution of nickel (II) chloride dimethoxyethane adduct (460 μg, 2.1 μmol) and 4,4′-di-tert-butyl-2,2′-bipyridine (560 μg, 2.1 μmol) in 10 mL of N,N-dimethylacetamide was then prepared. 1 mL of the catalyst solution was transferred to the reaction mixture and the mixture was sparged for 5 minutes with argon. The reaction vessel was placed in a water bath and irradiated by two 40 W Kessil LED Aquarium lights (A160WE Tuna Blue, 40 W each, 4 cm distance). The reaction was stirred under irradiation for 24 h. The reaction mixture was diluted with water. The solid was filtered, washed with water and dried under reduced pressure at 60° C. The crude material was then purified by preparative HPLC to give 38.9 mg (100% purity, 42% yield) of the title compound

LC-MS (method 2): Rₜ=1.32 min; MS (ESIpos): m/z=447 [M+H]⁺

¹H-NMR (400 MHz, DMSO-d6): δ [ppm]=1.26-1.40 (m, 1H), 1.48-1.61 (m, 1H), 1.82-1.96 (m, 2H), 2.07 (br d, 1H), 2.32-2.48 (m, 1H), 3.15-3.24 (m, 1H), 3.36-3.41 (m, 1H), 3.45 (s, 3H), 3.86 (s, 3H), 4.79 (br dd, 1H), 4.91 (s, 2H), 7.13 (d, 2H), 7.44 (t, 1H), 7.69 (d, 1H), 7.75 (d, 1H), 8.17-8.28 (m, 4H).

### Example 1004

### (3R)-3-{[7-(methoxymethyl)-2-(1-methyl-1H-pyrazol-4-yl)[1,2,4]triazolo[1,5-c]quinazolin-5-yl]amino}azepan-2-one

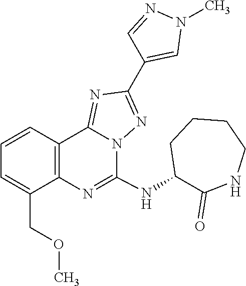 [SMILES: COCc1cccc2c1nc(N[C@@H]1CCCCNC1=O)n1nc(-c3cnn(C)c3)nc21]

The title compound was prepared similarly to example 1003 starting from (3R)-3-{[7-bromo-2-(1-methyl-1H-pyrazol-4-yl)[1,2,4]triazolo[1,5-c]quinazolin-5-yl]amino}azepan-2-one (89.9 mg, 197 μmol) in 18% yield (15.3 mg, 100% purity)

LC-MS (method 2): Rₜ=1.05 min; MS (ESIpos): m/z=421 [M+H]⁺

¹H-NMR (400 MHz, DMSO-d6): δ [ppm]=1.33 (br d, 1H), 1.52 (br d, 1H), 1.82-1.93 (m, 2H), 2.02-2.12 (m, 1H), 2.34-2.41 (m, 1H), 2.52-2.56 (m, 1H), 3.12-3.24 (m, 1H), 3.45 (s, 3H), 3.95 (s, 3H), 4.80 (br dd, 1H), 4.92 (s, 2H), 7.43 (t, 1H), 7.60 (br d, 1H), 7.72-7.79 (m, 1H), 8.07 (s, 1H), 8.18 (dd, 1H), 8.23 (dd, 1H), 8.50 (s, 1H).

### EXPERIMENTAL SECTION—BIOLOGICAL ASSAYS

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

The in vitro activity of the compounds of the present invention can be demonstrated in the following assays:

### Transactivation Assay in Human Cell Line (In Vitro Assays 1 and 2)

Transactivation assays were carried out in U87 MG glioblastoma cells (ATCC #HTB-14) endogenously expressing AHR. In addition the cells were stably transfected with an AHR inducible firefly luciferase reporter gene construct that carried AHR-binding sites (DRE) in its promoter and a renilla reporter gene construct with constitutively active promoter. Kynurenic acid is an endogenous AHR activating ligand and was used to prestimulate test cells prior to testing the antagonistic properties of compounds.

### In Vitro Assay 1: Antagonism in Human Cell Line

Cells in medium (tryptophan free RPMI (PAN-Biotech #P04-17598), 1% FCS (Biochrome Superior #S0615), 1×Penicillin/Streptomycin (Sigma P0781)) supplemented with 150 μM kynurenic acid were grown for 20 hours in absence (negative control) or presence of increasing concentrations of test compounds (typical dilutions: 72 pmol/L, 0.25 nmol/L, 0.89 nmol/L; 3.1 nmol/L, 11 nmol/L, 38 nmol/L, 130 nmol/L, 470 nmol/L, 1.6 μmol/L, 5.7 μmol/L and 20 μmol/L in duplicates). As positive inhibition control cells supplemented with 150 μM kynurenic acid were incubated in presence of 5 μM Staurosporin. Normalization was done by positive and negative controls.

Firefly luciferase and Renilla activity was determined by the DualGlo Luciferase Assay System (Promega, #2920). Renilla activity was used to assess toxic effects of compounds.

### In Vitro Assay 2: Agonism in Human Cell Line

Cells in medium (tryptophan free RPMI (PAN-Biotech #P04-17598), 1% FCS (Biochrome Superior #S0615), 1×Penicillin/Streptomycin (Sigma P0781)) were grown for 20 hours in absence (negative control) or presence of increasing concentrations of test compounds (typical dilutions: 72 μmol/L, 0.25 nmol/L, 0.89 nmol/L; 3.1 nmol/L, 11 nmol/L, 38 nmol/L, 130 nmol/L, 470 nmol/L, 1.6 μmol/L, 5.7 μmol/L and 20 μmol/L in duplicates). As positive activation control cells were incubated with 300 μM kynurenic acid. Normalization was done by positive and negative controls.

Firefly luciferase activity was determined by the SteadyGlo Luciferase Assay System (Promega, #E2520).

### Transactivation Assay in Mouse Cell Line (In Vitro Assays 3 and 4)

Transactivation assays were carried out in Hepa 1c1c7 cells (ATCC #CRL-2026) endogenously expressing AHR. In addition the cells were stably transfected with an AHR inducible firefly luciferase reporter gene construct that carried AHR-binding sites (DRE) in its promoter. Kynurenic acid is an endogenous AHR activating ligand and was used to prestimulate test cells prior to testing the antagonistic properties of compounds.

### In Vitro Assay 3: Antagonism in Mouse Cell Line

Cells in medium (tryptophan free RPMI (PAN-Biotech #P04-17598), 1% FCS (Biochrome Superior #S0615), 1×Penicillin/Streptomycin (Sigma P0781)) supplemented with 200 μM kynurenic acid (Sigma #K3375) were grown for 20 hours in absence (negative control) or presence of increasing concentrations of test compounds (typical dilutions: 72 μmol/L, 0.25 nmol/L, 0.89 nmol/L; 3.1 nmol/L, 11 nmol/L, 38 nmol/L, 130 nmol/L, 470 nmol/L, 1.6 μmol/L, 5.7 μmol/L and 20 μmol/L in duplicates). As positive inhibition control cells without addition of kynurenic acid were incubated. Normalization was done by positive and negative controls.

Firefly luciferase activity was determined by the SteadyGlo Luciferase Assay System (Promega, #E2550).

### In Vitro Assay 4: Agonism in Mouse Cell Line

Cells in medium (tryptophan free RPMI (PAN-Biotech #P04-17598), 1% FCS (Biochrome Superior #S0615), 1×Penicillin/Streptomycin (Sigma P0781)) were grown for 20 hours in absence (negative control) or presence of increasing concentrations of test compounds (typical dilutions: 72 μmol/L, 0.25 nmol/L, 0.89 nmol/L; 3.1 nmol/L, 11 nmol/L, 38 nmol/L, 130 nmol/L, 470 nmol/L, 1.6 μmol/L, 5.7 μmol/L and 20 μmol/L in duplicates). As positive activation control cells were incubated with 400 μM kynurenic acid. Normalization was done by positive and negative controls.

Firefly luciferase activity was determined by the SteadyGlo Luciferase Assay System (Promega, #2520).

TABLE 2
IC₅₀ values of examples in in vitro assays 1 and 3

| | AHR Antagonism | AHR Antagonism |
|---|---|---|
| Example | Human IC₅₀ [M] | Mouse IC₅₀ [M] |
| 1 | | 4.88E−09 |
| 2 | 2.40E−08 | 6.86E−08 |
| 3 | 1.82E−09 | 4.28E−10 |
| 4 | 1.68E−09 | 9.10E−10 |
| 5 | 3.40E−10 | |
| 6 | 5.93E−10 | >1.00E−5 |
| 7 | | 2.44E−09 |
| 8 | 5.44E−08 | 1.05E−07 |
| 9 | 2.62E−08 | 6.80E−08 |
| 10 | 2.71E−08 | 7.93E−09 |
| 11 | 1.36E−09 | 5.69E−10 |
| 12 | 1.31E−08 | 8.31E−09 |
| 13 | 1.21E−06 | 7.26E−06 |
| 14 | 2.14E−07 | 2.34E−07 |
| 15 | 1.78E−05 | 7.82E−06 |
| 16 | | >2.00E−5 |
| 17 | 7.44E−08 | 2.76E−08 |
| 18 | 1.11E−06 | 2.22E−06 |
| 19 | >2.00E−5 | |
| 20 | 1.24E−06 | 2.21E−06 |
| 21 | 1.29E−05 | >2.00E−5 |
| | | 1.97E−5 |
| 22 | 5.33E−06 | 2.03E−06 |
| 23 | >2.00E−5 | 3.38E−06 |
| | 1.65E−5 | |
| 24 | >4.00E−5 | |
| 25 | | 4.85E−07 |
| 26 | | 1.67E−08 |
| 27 | | 9.69E−09 |
| 28 | | 6.56E−09 |
| 29 | 4.03E−10 | 5.24E−10 |
| 30 | | 9.76E−09 |
| 31 | | 1.34E−08 |
| 32 | | |
| 33 | | |
| 34 | | 2.38E−08 |
| 35 | 1.55E−06 | 6.04E−08 |
| 36 | 8.01E−09 | 2.94E−08 |
| 37 | | 2.38E−07 |
| 38 | 1.94E−08 | 6.98E−08 |
| 39 | 1.59E−08 | 5.36E−08 |
| 40 | 2.08E−08 | 3.80E−07 |
| 41 | 2.12E−08 | 1.70E−09 |
| 42 | 3.03E−10 | 1.58E−10 |
| 43 | 8.65E−09 | 1.98E−08 |
| 44 | 1.57E−08 | 1.69E−08 |
| 45 | 1.19E−08 | 1.45E−08 |
| 46 | 2.93E−08 | 5.25E−09 |
| 47 | 1.86E−08 | 2.79E−08 |
| 48 | 3.20E−08 | 6.20E−08 |
| 49 | 8.50E−08 | 2.61E−08 |
| 50 | 1.06E−08 | 2.04E−08 |
| 51 | 3.09E−08 | 1.31E−07 |
| 52 | 1.30E−08 | 3.22E−09 |
| 53 | 1.16E−08 | 5.28E−09 |
| 54 | 2.08E−08 | 3.20E−09 |
| 55 | 2.01E−09 | 1.57E−08 |
| 56 | | 1.80E−10 |
| 57 | | |
| 58 | 1.05E−08 | |
| 59 | | |
| 60 | >2.00E−5 | 1.31E−07 |
| 61 | 3.63E−08 | 6.64E−07 |
| 62 | 1.11E−07 | 1.54E−07 |
| 63 | 2.87E−08 | 4.19E−08 |
| 64 | 6.33E−08 | 8.50E−08 |
| 65 | 6.42E−09 | 3.17E−09 |
| 66 | 1.36E−09 | 1.01E−09 |
| 67 | 6.43E−09 | 5.53E−09 |
| 68 | 1.03E−08 | 8.57E−09 |
| 69 | 3.59E−07 | 4.60E−07 |
| 70 | 2.44E−08 | 5.17E−08 |
| 71 | | 3.86E−09 |
| 72 | | 1.00E−08 |
| 73 | | 3.87E−09 |
| 74 | 1.62E−10 | |
| 75 | 2.57E−09 | 3.59E−10 |
| 76 | 4.82E−08 | 4.57E−09 |
| 77 | 6.52E−08 | 5.21E−08 |
| 78 | 4.65E−08 | 7.08E−08 |
| 79 | 4.90E−08 | 5.22E−08 |
| 80 | 2.53E−08 | |
| 81 | 4.02E−07 | 2.87E−07 |
| 82 | 8.87E−09 | 1.29E−08 |
| 83 | 6.99E−07 | 1.15E−07 |
| 84 | 1.01E−06 | 1.38E−06 |
| 85 | 4.95E−07 | 2.29E−07 |
| 86 | 5.10E−09 | 5.66E−09 |
| 87 | 4.85E−07 | 2.82E−07 |
| 88 | 2.11E−09 | 4.37E−10 |
| 89 | 1.29E−08 | 1.28E−08 |
| 90 | 4.54E−09 | 6.34E−09 |
| 91 | >2.00E−5 | >2.00E−5 |
| 92 | 8.63E−09 | 4.38E−09 |
| 93 | 1.52E−07 | 4.27E−07 |
| 94 | | 1.70E−08 |
| 95 | | 3.99E−07 |
| 96 | | 2.69E−07 |
| 97 | 1.56E−07 | 8.41E−08 |
| 98 | | 1.14E−06 |
| 99 | 6.78E−08 | |
| 100 | | |
| 101 | 3.48E−06 | 1.45E−07 |
| 102 | 6.01E−07 | 2.46E−07 |
| 103 | 4.43E−09 | 3.45E−10 |
| 104 | 1.12E−08 | 2.43E−09 |
| 105 | 8.86E−09 | 7.35E−09 |
| 106 | 7.97E−09 | 4.91E−09 |
| 107 | 1.06E−08 | 9.86E−09 |
| 108 | 5.75E−09 | 3.58E−09 |
| 109 | 6.94E−09 | 4.15E−09 |
| 110 | 1.01E−08 | 4.90E−09 |
| 111 | 1.42E−08 | 1.58E−08 |
| 112 | 3.79E−08 | 6.00E−09 |
| 113 | 1.58E−05 | 7.19E−07 |
| 114 | >2.00E−5 | 5.51E−6 |
| | | >5.71E−6 |
| 115 | 1.11E−06 | 6.80E−08 |
| 116 | 2.75E−06 | 7.33E−08 |
| 117 | 9.04E−08 | 7.65E−08 |
| 118 | 6.43E−08 | 4.02E−08 |
| 119 | 7.80E−07 | 2.02E−07 |
| 120 | 6.02E−08 | 5.61E−08 |
| 121 | 1.96E−07 | 2.75E−07 |
| 122 | 5.55E−08 | 2.61E−08 |
| 123 | 3.65E−07 | 1.87E−06 |
| 124 | 2.83E−08 | 5.41E−08 |
| 125 | 5.68E−10 | 4.64E−09 |
| 126 | 1.30E−08 | 1.48E−08 |
| 127 | | |
| 128 | 1.50E−09 | 3.16E−10 |
| 129 | | |
| 130 | 4.55E−09 | 5.82E−09 |
| 131 | 1.79E−07 | 9.38E−08 |
| 132 | 2.15E−08 | 2.63E−08 |
| 133 | 1.92E−08 | 3.27E−08 |
| 134 | 8.35E−08 | 8.68E−08 |
| 135 | 2.77E−08 | 2.05E−08 |
| 136 | 3.09E−08 | 9.58E−09 |
| 137 | 2.16E−09 | 8.23E−10 |
| 138 | 7.41E−10 | 1.45E−09 |
| 139 | | 2.58E−10 |
| 140 | 1.09E−09 | 1.08E−09 |
| 141 | 2.84E−08 | 8.32E−08 |
| 142 | 2.15E−09 | 2.11E−08 |
| 143 | 2.42E−10 | 8.84E−10 |
| 144 | >2.00E−6 | 6.27E−08 |
| 145 | 3.17E−08 | |
| 146 | | |
| 147 | 7.53E−07 | |
| 148 | 8.12E−07 | 2.41E−09 |
| 149 | 1.81E−07 | |
| 150 | 1.97E−07 | 4.88E−08 |
| 151 | 2.36E−06 | 3.43E−07 |
| 152 | 5.28E−08 | 3.53E−07 |
| 153 | 4.35E−10 | 1.18E−09 |
| 154 | 2.18E−09 | 1.16E−09 |
| 155 | | |
| 156 | | 1.42E−08 |
| 157 | | 4.54E−08 |
| 158 | 1.77E−08 | 3.22E−08 |
| 159 | 1.03E−09 | 2.27E−10 |
| 160 | 1.43E−07 | 1.54E−08 |
| 161 | | |
| 162 | 2.23E−08 | 2.55E−08 |
| 163 | 2.14E−08 | |
| 164 | 2.44E−09 | 4.54E−09 |
| 165 | 1.65E−08 | 9.86E−08 |
| 166 | | 2.57E−09 |
| 167 | 6.39E−09 | |
| 168 | 9.09E−10 | 2.21E−09 |
| 169 | 7.27E−09 | 1.18E−07 |
| 170 | | 5.84E−10 |
| 171 | 6.20E−08 | |
| 172 | 1.29E−08 | 1.20E−08 |
| 173 | 7.21E−08 | 1.12E−07 |
| 174 | 2.26E−08 | 4.04E−09 |
| 175 | 3.24E−08 | 2.23E−08 |
| 176 | 6.81E−09 | 6.55E−09 |
| 177 | 7.35E−07 | |
| 178 | 3.98E−09 | 2.34E−09 |
| 179 | 1.01E−07 | 2.46E−07 |
| 180 | 1.27E−07 | 2.53E−07 |
| 181 | 3.37E−07 | 2.57E−08 |
| 182 | 2.54E−07 | 1.02E−06 |
| 183 | 2.48E−08 | 4.53E−07 |
| 184 | 9.66E−09 | 6.28E−08 |
| 185 | 1.05E−08 | 9.84E−09 |
| 186 | 2.09E−08 | 2.76E−08 |
| 187 | 6.04E−08 | 3.21E−07 |
| 188 | 3.00E−08 | 5.04E−08 |
| 189 | 2.61E−07 | 1.14E−07 |
| 190 | 1.54E−07 | 2.52E−07 |
| 191 | 1.96E−07 | 1.09E−06 |
| 192 | | 3.49E−08 |
| 193 | 2.05E−08 | 8.72E−08 |
| 194 | 1.17E−09 | 3.83E−09 |
| 195 | 3.06E−09 | 2.55E−09 |
| 196 | 4.09E−09 | 1.11E−09 |
| 197 | 4.23E−09 | 1.82E−09 |
| 198 | 1.82E−06 | |
| 199 | 5.37E−07 | 1.14E−07 |
| 200 | 2.87E−09 | >2.00E−5 |
| 201 | 3.56E−10 | |
| 202 | 5.17E−10 | 6.57E−10 |
| 203 | 6.29E−10 | 4.80E−10 |
| 204 | 1.15E−09 | 1.02E−09 |
| 205 | 2.15E−09 | 7.65E−09 |
| 206 | 9.97E−09 | 7.88E−09 |
| 207 | 1.77E−08 | 2.42E−07 |
| 208 | 1.34E−06 | 2.38E−08 |
| 209 | | 1.55E−08 |
| 210 | | 5.01E−09 |
| 211 | | 1.67E−08 |
| 212 | 9.71E−10 | 2.49E−09 |
| 213 | | |
| 214 | | |
| 215 | | 1.23E−09 |
| 216 | 3.35E−09 | 1.21E−08 |
| 217 | | 2.54E−09 |
| 218 | 8.68E−09 | 2.94E−08 |
| 219 | 8.15E−08 | 7.14E−09 |
| 220 | 2.00E−08 | 6.76E−09 |
| 221 | 3.46E−07 | 6.53E−07 |
| 222 | | |
| 223 | 5.32E−08 | 1.11E−07 |
| 224 | | |
| 225 | | |
| 226 | | 1.53E−09 |
| 227 | 2.65E−06 | |
| 228 | 3.01E−08 | 3.89E−08 |
| 229 | 5.82E−08 | 1.01E−07 |
| 230 | 1.97E−06 | |
| 231 | 8.14E−10 | 4.06E−10 |
| 232 | 8.83E−09 | 3.88E−09 |
| 233 | | |
| 234 | 1.70E−09 | 1.40E−09 |
| 235 | 2.34E−09 | 1.14E−09 |
| 236 | 2.62E−08 | 2.12E−08 |
| 237 | 1.33E−08 | 3.87E−08 |
| 238 | 1.69E−07 | 3.44E−07 |
| 239 | 8.92E−09 | 1.02E−08 |
| 240 | 4.08E−08 | 1.71E−07 |
| 241 | 1.27E−09 | 1.93E−09 |
| 242 | 3.06E−09 | 3.02E−09 |
| 243 | 4.02E−09 | 1.48E−08 |
| 244 | 2.17E−08 | 6.67E−08 |
| 245 | 3.21E−08 | 3.10E−07 |
| 246 | 3.32E−09 | 1.12E−08 |
| 247 | 3.08E−07 | 5.91E−07 |
| 248 | 5.71E−09 | 1.82E−09 |
| 249 | 2.96E−08 | 3.63E−09 |
| 250 | 1.43E−08 | 1.35E−07 |
| 251 | 3.04E−07 | |
| 252 | 6.55E−08 | 4.98E−08 |
| 253 | 2.71E−08 | 3.68E−08 |
| 254 | | |
| 255 | 4.98E−08 | 5.10E−09 |
| 256 | | 6.94E−09 |
| 257 | 6.29E−06 | 1.21E−05 |
| 258 | 7.15E−07 | 3.98E−07 |
| 259 | | 6.44E−10 |
| 260 | | 6.75E−09 |
| 261 | 7.39E−08 | 4.66E−08 |
| 262 | 1.37E−06 | 8.57E−08 |
| 263 | 3.86E−07 | 8.93E−09 |
| 264 | >2.00E−5 | >2.00E−5 |
| 265 | 2.38E−07 | 3.73E−07 |
| 266 | 3.29E−09 | 2.17E−09 |
| 267 | | 2.47E−08 |
| 268 | 6.36E−09 | 3.07E−09 |
| 269 | | 1.20E−09 |
| 270 | 7.93E−10 | 9.46E−10 |
| 271 | 7.02E−07 | 1.39E−05 |
| 272 | 2.77E−06 | |
| 273 | >2.00E−5 | 4.59E−06 |
| 274 | 1.09E−06 | 2.15E−06 |
| 275 | | |
| 276 | 1.45E−08 | 1.64E−08 |
| 277 | >2.00E−5 | 1.51E−08 |
| 278 | 3.72E−07 | 5.10E−08 |
| 279 | 3.08E−07 | 1.80E−07 |
| 280 | | 3.33E−08 |
| 281 | | 2.36E−08 |
| 282 | | 2.69E−08 |
| 283 | | 1.72E−07 |
| 284 | >2.00E−5 | 2.77E−07 |
| 285 | 1.36E−06 | 6.25E−07 |
| 286 | 2.78E−06 | 3.35E−06 |
| 287 | 6.55E−06 | 7.56E−07 |
| 288 | >5.71E−6 | 8.87E−07 |
| | >2.00E−5 | |
| 289 | 1.12E−06 | 2.69E−07 |
| 290 | | 2.88E−07 |
| 291 | 2.65E−06 | 2.68E−06 |
| 292 | | 4.03E−08 |
| 293 | | 5.61E−08 |
| 294 | | 5.43E−06 |
| 295 | >2.00E−5 | 2.91E−06 |
| 296 | | 7.31E−08 |
| 297 | | 2.51E−08 |
| 298 | | 6.33E−08 |
| 299 | | 3.73E−08 |
| 300 | >2.00E−5 | 6.18E−06 |
| 301 | >2.00E−5 | 2.24E−06 |
| 302 | | |
| 303 | 4.52E−07 | 2.93E−07 |
| 304 | 2.86E−06 | 1.83E−06 |
| 305 | 2.97E−07 | 1.78E−07 |
| 306 | 1.09E−07 | 3.12E−08 |
| 307 | 1.35E−07 | 8.47E−08 |
| 308 | 1.10E−07 | 1.86E−08 |
| 309 | 2.47E−08 | 1.97E−09 |
| 310 | 9.98E−08 | 6.07E−08 |
| 311 | 1.36E−07 | 1.11E−07 |
| 312 | 7.19E−08 | 2.20E−08 |
| 313 | 1.06E−07 | 9.92E−08 |
| 314 | 6.90E−09 | 9.22E−10 |
| 315 | 2.06E−07 | 5.08E−08 |
| 316 | 2.07E−08 | 3.76E−09 |
| 317 | 1.45E−07 | |
| 318 | 1.29E−08 | 6.03E−10 |
| 319 | 1.74E−08 | 5.02E−10 |
| 320 | 1.21E−06 | |
| 321 | 2.06E−08 | 1.24E−08 |
| 322 | 6.42E−08 | 3.57E−08 |
| 323 | >2.00E−5 | |
| | >2.00E−5 | |
| | 1.32E−5 | |
| | 8.31E−6 | |
| 324 | 2.02E−08 | 1.64E−08 |
| 325 | 1.49E−5 | 6.77E−07 |
| | 1.56E−5 | |
| | >2.00E−5 | |
| | >2.00E−5 | |
| 326 | | |
| 327 | | 5.29E−09 |
| 328 | | 1.93E−08 |
| 329 | 5.94E−09 | 2.89E−09 |
| 330 | | |
| 331 | | |
| 332 | | 6.04E−09 |
| 333 | 2.74E−08 | 2.80E−08 |
| 334 | | |
| 335 | | |
| 336 | 8.51E−06 | 4.34E−07 |
| 337 | 1.71E−06 | >2.00E−5 |
| 338 | 1.91E−07 | 5.39E−08 |
| 339 | 3.37E−07 | 1.69E−07 |
| 340 | 7.33E−10 | 5.17E−10 |
| 341 | 1.18E−07 | 1.77E−08 |
| 342 | 2.21E−07 | 1.75E−07 |
| 343 | 6.67E−08 | 1.78E−07 |
| 344 | 3.57E−09 | 6.28E−10 |
| 345 | 1.24E−07 | 2.70E−08 |
| 346 | 2.24E−09 | |
| 347 | 1.17E−07 | 4.28E−09 |
| 348 | 7.76E−08 | 3.74E−08 |
| 349 | 5.16E−08 | 1.15E−08 |
| 350 | 4.13E−08 | 3.59E−09 |
| 351 | 1.37E−09 | 1.25E−10 |
| 352 | 9.72E−10 | |
| 353 | 2.36E−07 | 2.74E−08 |
| 354 | 4.05E−08 | 7.82E−08 |
| 355 | 3.79E−08 | 3.66E−08 |
| 356 | 2.71E−08 | 3.04E−08 |
| 357 | 1.35E−09 | |
| 358 | 3.15E−09 | 1.78E−09 |
| 359 | >2.00E−5 | 9.34E−08 |
| 360 | >2.00E−5 | 5.44E−06 |
| 361 | >2.00E−5 | 6.68E−08 |
| 362 | 1.91E−08 | 3.54E−09 |
| 363 | 3.50E−08 | 1.07E−08 |
| 364 | | 2.47E−07 |
| 365 | | |
| 366 | >2.00E−5 | 5.93E−07 |
| 367 | 1.91E−07 | 1.05E−08 |
| 368 | 5.00E−08 | 2.48E−08 |
| 369 | 1.64E−08 | 9.97E−09 |
| 370 | 5.45E−08 | 6.38E−09 |
| 371 | 7.24E−08 | 1.46E−08 |
| 372 | 8.65E−09 | 1.73E−09 |
| 373 | 6.38E−07 | 5.14E−07 |
| 374 | 4.49E−07 | 2.11E−07 |
| 375 | 7.95E−07 | 3.06E−08 |
| 376 | 1.61E−08 | 4.93E−09 |
| 377 | 3.28E−08 | 1.24E−08 |
| 378 | 6.25E−07 | 1.71E−07 |
| 379 | 7.60E−10 | 9.93E−10 |
| 381 | 1.02E−08 | 1.51E−09 |
| 382 | 7.64E−09 | 1.25E−09 |
| 383 | 1.39E−07 | 3.88E−07 |
| 384 | 2.73E−07 | 7.22E−08 |
| 385 | 1.97E−06 | 1.46E−07 |
| 386 | 6.02E−10 | 3.46E−10 |
| 387 | 7.93E−10 | 3.98E−10 |
| 388 | | 2.89E−06 |
| 389 | 1.76E−08 | 2.67E−08 |
| 390 | 1.38E−09 | 1.02E−09 |
| 391 | 3.31E−08 | 4.61E−08 |
| 392 | 2.38E−07 | 3.15E−08 |
| 393 | 4.34E−08 | 3.42E−08 |
| 394 | 1.16E−08 | 3.09E−08 |
| 395 | 3.00E−07 | 2.41E−06 |
| 396 | 1.12E−08 | 2.16E−08 |
| 397 | 1.83E−08 | 3.76E−08 |
| 398 | 2.24E−08 | 1.35E−07 |
| 399 | 1.85E−08 | 5.57E−08 |
| 400 | 4.16E−08 | 1.19E−07 |
| 401 | 4.44E−09 | 8.70E−09 |
| 402 | 8.32E−09 | 2.61E−08 |
| 403 | 3.03E−08 | 9.51E−08 |
| 404 | 3.39E−06 | 2.76E−06 |
| 405 | 3.41E−07 | 7.92E−07 |
| 406 | 1.01E−07 | 7.75E−06 |
| 407 | 3.10E−07 | 2.46E−07 |
| 408 | 1.85E−07 | 1.36E−08 |
| 409 | 6.98E−08 | 1.71E−08 |
| 410 | 1.53E−08 | 4.73E−09 |
| 411 | 1.77E−07 | 1.12E−08 |
| 412 | 8.37E−08 | 2.36E−07 |
| 413 | 8.94E−08 | 6.21E−08 |
| 414 | | >2.00E−5 |
| 415 | 1.85E−08 | 6.47E−09 |
| 416 | 2.10E−06 | 3.54E−07 |
| 417 | 1.11E−06 | 7.30E−06 |
| 418 | | 8.71E−06 |
| 419 | 3.49E−09 | 3.46E−09 |
| 420 | 9.36E−10 | 8.87E−10 |
| 421 | 7.09E−10 | 6.90E−10 |
| 422 | 3.61E−10 | 9.27E−10 |
| 423 | 2.81E−10 | 3.49E−10 |
| 424 | 5.89E−10 | 1.69E−09 |
| 425 | 6.84E−09 | 2.39E−08 |
| 426 | 1.84E−08 | 1.43E−08 |
| 427 | | 3.16E−09 |
| 428 | 2.44E−06 | 5.61E−07 |
| 429 | 7.80E−09 | 7.09E−08 |
| 430 | 3.22E−07 | |
| 431 | 1.43E−08 | 6.06E−08 |
| 432 | 6.11E−08 | 1.81E−08 |
| 433 | 1.64E−09 | 6.47E−10 |
| 434 | 6.43E−08 | 7.50E−08 |
| 435 | 1.30E−07 | 1.64E−07 |
| 436 | | |
| 437 | 3.65E−08 | 7.87E−09 |
| 438 | 2.90E−07 | 1.30E−07 |
| 439 | 4.52E−08 | 2.70E−08 |
| 440 | 2.43E−09 | 6.42E−10 |
| 441 | 1.36E−07 | 2.93E−08 |
| 442 | 4.89E−06 | 2.64E−07 |
| 443 | 1.02E−08 | 6.72E−10 |
| 444 | | |
| 445 | 9.27E−08 | |
| 446 | 2.92E−09 | 5.10E−10 |
| 447 | 2.27E−08 | 1.13E−08 |
| 448 | | 1.34E−07 |
| 449 | 1.73E−08 | 2.10E−08 |
| 450 | >2.00E−5 | 8.57E−06 |
| 451 | 7.16E−06 | 1.31E−07 |
| 452 | 3.98E−09 | 3.35E−09 |
| 453 | | 1.75E−08 |
| 454 | | 1.65E−07 |
| 455 | 1.17E−07 | |
| 456 | 2.98E−09 | 3.56E−09 |
| 457 | 9.89E−07 | 7.83E−08 |
| 458 | 6.37E−08 | 2.80E−08 |
| 459 | 7.90E−08 | 3.02E−08 |
| 460 | 7.67E−08 | |
| 461 | 6.18E−09 | 1.95E−09 |
| 462 | | 8.12E−08 |
| 463 | 2.94E−08 | 1.88E−09 |
| 464 | 5.89E−08 | 3.91E−09 |
| 465 | 1.34E−08 | 3.87E−09 |
| 466 | 1.26E−07 | 1.07E−07 |
| 467 | | 3.28E−08 |
| 468 | 6.73E−08 | 4.27E−08 |
| 469 | 1.64E−08 | 1.20E−09 |
| 470 | 1.34E−09 | 5.00E−10 |
| 471 | 5.60E−08 | 5.26E−09 |
| 472 | 3.72E−08 | 3.81E−08 |
| 473 | 5.10E−08 | 1.79E−08 |
| 474 | 3.11E−09 | 1.37E−09 |
| 475 | >2.00E−5 | 7.17E−08 |
| 476 | 3.93E−09 | 5.54E−10 |
| 477 | 1.81E−08 | 1.44E−09 |
| 478 | 4.98E−08 | 2.99E−09 |
| 479 | 3.53E−08 | 3.32E−08 |
| 480 | 2.07E−08 | 1.48E−08 |
| 481 | 1.02E−06 | 1.04E−07 |
| 482 | 2.23E−06 | 3.16E−06 |
| 483 | 1.45E−06 | 2.17E−06 |
| 484 | 9.06E−08 | 2.76E−08 |
| 485 | 7.54E−07 | 1.01E−05 |
| 486 | 3.34E−07 | 1.84E−07 |
| 487 | 3.26E−07 | 2.00E−07 |
| 488 | 6.40E−08 | 1.23E−08 |
| 489 | 4.24E−07 | 4.33E−08 |
| 490 | 4.25E−08 | 5.06E−08 |
| 491 | 9.72E−08 | 5.05E−08 |
| 492 | 2.22E−08 | 5.71E−09 |
| 493 | >2.00E−5 | 3.69E−08 |
| 494 | 2.28E−07 | 1.11E−08 |
| 495 | 7.03E−08 | 1.32E−08 |
| 496 | 4.66E−08 | 1.90E−09 |
| 497 | | 5.71E−08 |
| 498 | >2.00E−5 | 7.00E−07 |
| 499 | >2.00E−5 | >2.00E−5 |
| | >2.00E−6 | |
| 500 | >2.00E−5 | 2.97E−06 |
| 501 | >2.00E−5 | 9.08E−08 |
| 502 | | 1.96E−07 |
| 503 | >2.00E−5 | >2.00E−5 |
| 504 | 3.65E−07 | 3.79E−08 |
| 505 | >2.00E−5 | 5.07E−08 |
| | >2.00E−6 | |
| | >5.71E−7 | |
| 506 | >2.00E−5 | >2.00E−5 |
| 507 | 3.44E−07 | 4.12E−08 |
| 508 | 3.53E−09 | 4.21E−09 |
| 509 | 1.03E−07 | 5.69E−08 |
| 510 | 5.39E−08 | 1.17E−08 |
| 511 | 7.50E−09 | 1.40E−09 |
| 512 | 1.04E−08 | 2.48E−09 |
| 513 | 2.66E−07 | 3.56E−08 |
| 514 | >2.00E−5 | 1.88E−07 |
| 515 | 3.17E−08 | 2.06E−09 |
| 516 | 4.64E−09 | 2.18E−09 |
| 517 | 1.47E−09 | 2.31E−09 |
| 518 | 4.50E−08 | 7.94E−09 |
| 519 | 3.65E−08 | 2.22E−08 |
| 520 | 3.05E−08 | 9.59E−09 |
| 521 | 2.04E−08 | 1.22E−08 |
| 522 | >2.00E−5 | 2.91E−08 |
| 523 | 4.99E−09 | 8.40E−10 |
| 524 | 1.30E−07 | 1.77E−08 |
| 525 | 9.92E−10 | |
| 526 | 1.47E−08 | 2.33E−09 |
| 527 | | 5.53E−09 |
| 528 | 9.30E−09 | 6.94E−09 |
| 529 | 2.80E−09 | |
| 530 | >2.00E−5 | |
| 531 | 1.29E−09 | |
| 532 | | 1.06E−08 |
| 533 | 3.72E−09 | 3.94E−09 |
| 534 | 2.03E−09 | 8.28E−10 |
| 535 | 5.13E−09 | 2.57E−09 |
| 536 | 8.60E−08 | 9.47E−08 |
| 537 | 4.31E−09 | 1.90E−09 |
| 538 | 8.79E−10 | 6.46E−10 |
| 539 | 6.16E−08 | 7.71E−09 |
| 540 | 6.05E−08 | 6.24E−09 |
| 541 | 3.50E−08 | 6.31E−07 |
| 542 | 3.55E−09 | 5.65E−10 |
| 543 | | |
| 544 | | |
| 545 | | >2.00E−5 |
| 546 | 2.24E−06 | |
| 547 | >5.71E−6 | |
| 548 | 9.46E−07 | 2.40E−06 |
| 549 | 7.33E−08 | 1.74E−05 |
| 550 | 5.35E−06 | |
| 551 | 1.17E−08 | 1.38E−08 |
| 552 | 1.28E−07 | 2.30E−07 |
| 553 | | 1.18E−09 |
| 554 | 7.68E−09 | 2.45E−09 |
| 555 | 1.82E−08 | 6.92E−09 |
| 556 | 4.03E−10 | 4.15E−10 |
| 557 | 2.39E−10 | 8.65E−10 |
| 558 | 5.96E−09 | 2.76E−10 |
| 559 | 5.14E−09 | 1.04E−08 |
| 560 | 3.80E−09 | 5.57E−09 |
| 561 | 3.96E−09 | 7.55E−09 |
| 562 | 1.37E−09 | 2.98E−09 |
| 563 | | |
| 564 | 1.23E−08 | 1.75E−08 |
| 565 | 8.44E−09 | 3.33E−09 |
| 566 | 1.76E−07 | 2.08E−08 |
| 567 | 1.21E−08 | 3.05E−09 |
| 568 | 3.31E−07 | 2.99E−08 |
| 569 | 8.40E−09 | 4.30E−09 |
| 570 | 8.08E−07 | 8.07E−08 |
| 571 | >2.00E−5 | 1.01E−08 |
| 572 | >2.00E−5 | 1.31E−07 |
| 573 | 3.36E−09 | 1.39E−09 |
| 574 | 5.00E−09 | 1.07E−09 |
| 575 | | |
| 576 | 1.77E−08 | 2.96E−09 |
| 577 | 6.60E−10 | 2.81E−10 |
| 578 | 1.25E−08 | 2.91E−09 |
| 579 | 1.13E−08 | 6.81E−08 |
| 580 | 5.40E−08 | 6.14E−08 |
| 581 | 1.69E−09 | |
| 582 | | 4.41E−08 |
| 583 | 9.48E−10 | 3.11E−09 |
| 584 | 4.39E−08 | 1.53E−07 |
| 585 | 6.91E−08 | 8.30E−07 |
| 586 | 2.79E−07 | |
| 587 | 7.70E−09 | 2.06E−08 |
| 588 | 1.84E−08 | 1.06E−06 |
| 589 | >2.00E−5 | |
| 590 | 3.65E−07 | |
| 591 | | 2.42E−06 |
| 592 | 5.21E−07 | >2.00E−5 |
| 593 | | 8.21E−08 |
| 594 | 5.01E−07 | >2.00E−5 |
| 595 | 5.18E−08 | 3.40E−08 |
| 596 | 4.08E−06 | 3.14E−06 |
| 597 | 1.16E−08 | 7.57E−09 |
| 598 | 2.80E−07 | 1.71E−07 |
| 599 | 5.81E−07 | 2.12E−06 |
| 600 | 4.48E−08 | 3.13E−08 |
| 601 | 2.96E−07 | 1.58E−07 |
| 602 | 6.81E−09 | 2.69E−09 |
| 603 | 4.08E−09 | 3.39E−09 |
| 604 | 1.33E−07 | 3.47E−07 |
| 605 | 1.59E−09 | 1.05E−09 |
| 606 | 9.02E−09 | 3.08E−09 |
| 607 | 1.00E−08 | 1.31E−08 |
| 608 | 1.12E−07 | 2.55E−08 |
| 609 | 1.57E−07 | 3.24E−07 |
| 610 | 2.44E−07 | 8.65E−08 |
| 611 | 4.36E−09 | 3.16E−09 |
| 612 | 1.32E−09 | 1.23E−09 |
| 613 | 1.17E−09 | 7.99E−10 |
| 614 | 2.09E−08 | 2.38E−08 |
| 615 | | |
| 616 | 7.60E−10 | 1.07E−09 |
| 617 | | |
| 618 | | |
| 619 | 1.44E−09 | 2.96E−09 |
| 620 | 8.44E−08 | |
| 621 | 1.02E−09 | 9.64E−10 |
| 622 | | |
| 623 | 3.37E−07 | 4.65E−09 |
| 624 | 3.49E−09 | 1.24E−09 |
| 625 | 1.50E−09 | >2.00E−6 |
| 626 | 9.20E−10 | |
| 627 | 6.68E−10 | 3.26E−10 |
| 628 | 1.20E−09 | 1.03E−09 |
| 629 | 4.88E−08 | 1.90E−07 |
| 630 | 4.83E−10 | 8.61E−10 |
| 631 | 1.00E−09 | 3.53E−09 |
| 632 | 1.29E−09 | 7.84E−10 |
| 633 | 1.14E−09 | 6.40E−10 |
| 634 | 9.09E−08 | 2.06E−07 |
| 635 | 9.49E−10 | 1.10E−09 |
| 636 | 2.32E−08 | 5.98E−09 |
| 637 | 1.15E−08 | 2.14E−08 |
| 638 | 4.00E−09 | 1.04E−08 |
| 639 | | 9.27E−10 |
| 640 | 2.54E−08 | 3.07E−08 |
| 641 | 6.85E−09 | 2.46E−09 |
| 642 | 3.34E−07 | 6.83E−07 |
| 643 | | |
| 644 | 1.03E−07 | 6.17E−08 |
| 645 | 1.66E−08 | 6.64E−09 |
| 646 | 2.89E−06 | >2.00E−5 |
| | | 1.18E−5 |
| 647 | 6.34E−06 | >2.00E−5 |
| | | 1.28E−5 |
| 648 | 6.89E−09 | 3.63E−09 |
| 649 | 3.80E−07 | 2.73E−08 |
| 650 | 1.10E−07 | 6.71E−08 |
| 653 | 2.58E−07 | >2.00E−5 |
| 654 | 1.03E−07 | 2.21E−07 |
| 655 | 1.39E−07 | 2.56E−07 |
| 656 | | 3.01E−08 |
| 657 | 1.40E−08 | 8.21E−08 |
| 658 | | 4.50E−07 |
| 659 | 1.62E−06 | 4.50E−06 |
| 660 | 8.48E−07 | 9.27E−07 |
| 661 | 1.53E−07 | 1.39E−07 |
| 662 | 6.86E−08 | 7.04E−08 |
| 663 | >2.00E−5 | 3.17E−07 |
| 664 | 1.26E−08 | 1.47E−09 |
| 665 | 1.87E−08 | 1.13E−08 |
| 666 | | |
| 667 | 2.68E−09 | 4.56E−09 |
| 668 | >5.71E−6 | 2.71E−07 |
| 669 | 1.99E−09 | 4.96E−09 |
| 670 | 2.78E−07 | 1.60E−07 |
| 671 | | 7.44E−10 |
| 672 | | 4.35E−09 |
| 673 | >2.00E−5 | 1.57E−09 |
| 674 | 3.44E−10 | 6.69E−10 |
| 675 | 1.22E−09 | 2.26E−09 |
| 676 | 6.18E−10 | 1.97E−09 |
| 677 | 1.33E−08 | 5.31E−08 |
| 678 | | |
| 679 | | |
| 680 | 3.93E−08 | 3.56E−08 |
| 681 | 1.45E−08 | 1.31E−08 |
| 682 | 7.43E−08 | 6.82E−09 |
| 683 | >5.71E−7 | 1.66E−08 |
| 684 | 3.39E−09 | 8.96E−10 |
| 685 | >2.00E−6 | 1.42E−07 |
| 686 | 2.10E−07 | 1.85E−08 |
| 687 | >2.00E−6 | 9.78E−08 |
| 688 | >2.00E−5 | >2.00E−5 |
| 689 | 1.60E−07 | 1.56E−07 |
| 690 | >2.00E−5 | >2.00E−5 |
| 691 | 5.61E−07 | 2.40E−07 |
| 692 | 6.79E−10 | 3.08E−10 |
| 693 | 7.43E−09 | 2.52E−09 |
| 694 | 1.55E−07 | 7.19E−07 |
| 695 | 2.96E−06 | 2.19E−06 |
| 696 | 3.23E−09 | 8.97E−09 |
| 697 | 6.22E−09 | 1.19E−09 |
| 698 | 4.68E−08 | 7.35E−09 |
| 699 | 9.83E−10 | 1.47E−09 |
| 700 | | |
| 701 | | |
| 702 | | |
| 703 | | |
| 704 | | |
| 705 | | |
| 706 | | |
| 707 | | |
| 708 | | |
| 709 | | |
| 710 | 4.84E−09 | 1.80E−09 |
| 711 | | |
| 712 | | |
| 713 | | |
| 714 | 1.64E−07 | 1.34E−07 |
| 715 | 1.13E−08 | 1.48E−08 |
| 716 | | |
| 717 | 7.05E−10 | 9.50E−10 |
| 718 | 1.93E−08 | 8.12E−09 |
| 719 | 9.56E−06 | 2.39E−07 |
| 720 | 1.45E−07 | 6.38E−08 |
| 721 | 2.47E−09 | 1.06E−08 |
| 722 | 7.20E−09 | 6.63E−09 |
| 723 | | |
| 724 | | |
| 725 | | |
| 726 | 1.09E−05 | |
| 727 | | |
| 728 | >2.00E−5 | 4.46E−07 |
| 729 | 3.60E−06 | 1.61E−07 |
| 730 | n.d. | >2.00E−5 |
| 731 | n.d. | n.d. |
| 732 | 1.32E−09 | 6.14E−09 |
| 733 | 2.58E−07 | 6.64E−06 |
| 734 | 3.94E−09 | 8.92E−09 |
| 735 | 7.10E−09 | 9.20E−09 |
| 736 | 7.84E−10 | 6.69E−10 |
| 737 | 7.64E−09 | 1.51E−08 |
| 738 | 3.40E−09 | 1.03E−09 |
| 739 | 6.64E−10 | 1.40E−09 |
| 740 | 1.22E−09 | 1.65E−09 |
| 741 | 7.02E−10 | 1.41E−09 |
| 742 | 8.31E−10 | 1.90E−09 |
| 743 | 1.26E−09 | 1.73E−09 |
| 744 | 1.69E−09 | 1.16E−08 |
| 745 | 4.41E−09 | 1.14E−09 |
| 746 | 7.48E−09 | 4.56E−08 |
| 747 | 1.65E−09 | n.d. |
| 748 | 3.90E−10 | 6.56E−10 |
| 749 | 1.54E−09 | 2.12E−09 |
| 750 | 3.56E−09 | 1.41E−09 |
| 751 | 1.47E−08 | 5.47E−08 |
| 752 | 2.29E−07 | 9.53E−07 |
| 753 | 9.89E−09 | 4.06E−08 |
| 754 | 7.97E−08 | 2.92E−07 |
| 755 | 9.19E−06 | n.d. |
| 756 | 4.49E−06 | 1.17E−05 |
| 757 | 5.40E−08 | 1.09E−07 |
| 758 | 1.35E−08 | 1.96E−08 |
| 759 | 7.68E−09 | 2.50E−08 |
| 760 | 3.60E−09 | 1.00E−08 |
| 761 | 3.11E−09 | 9.93E−09 |
| 762 | 2.44E−09 | 7.01E−09 |
| 763 | 1.41E−09 | 3.96E−09 |
| 764 | 1.86E−09 | 1.73E−09 |
| 765 | 7.79E−07 | 5.85E−06 |
| 766 | 1.64E−08 | 4.70E−08 |
| 767 | 2.69E−08 | 4.76E−08 |
| 768 | 1.32E−07 | 1.22E−06 |
| 769 | 2.10E−08 | 1.51E−07 |
| 770 | 4.04E−09 | 5.09E−09 |
| 771 | 1.02E−08 | 1.04E−07 |
| 772 | 3.61E−09 | 5.13E−08 |
| 773 | 8.57E−10 | 9.95E−10 |
| 774 | 2.26E−06 | 5.55E−07 |
| 775 | 3.13E−06 | 7.43E−07 |
| 776 | 2.92E−06 | 1.02E−06 |
| 777 | 1.80E−09 | 2.21E−09 |
| 778 | 2.09E−09 | 4.32E−09 |
| 779 | 4.30E−09 | 5.96E−09 |
| 780 | 4.91E−09 | 5.55E−09 |
| 781 | 1.17E−09 | 2.09E−09 |
| 782 | 1.29E−07 | 7.97E−08 |
| 783 | 1.71E−07 | 2.91E−07 |
| 784 | 9.16E−08 | 4.81E−08 |
| 785 | 2.38E−08 | 3.22E−08 |
| 786 | 2.20E−08 | 2.21E−08 |
| 787 | 2.26E−08 | 3.82E−08 |
| 788 | 4.64E−10 | 2.08E−09 |
| 789 | 4.30E−08 | 1.71E−08 |
| 790 | 4.14E−07 | 4.02E−07 |
| 791 | 5.23E−07 | 2.64E−07 |
| 792 | 6.60E−08 | 2.87E−08 |
| 793 | 2.94E−06 | 6.94E−07 |
| 794 | 1.76E−08 | 2.12E−08 |
| 795 | 2.46E−07 | 3.13E−06 |
| 796 | 1.37E−08 | 2.02E−08 |
| 797 | 1.58E−09 | 9.63E−10 |
| 798 | 7.57E−10 | 8.11E−10 |
| 799 | 4.21E−08 | 1.85E−08 |
| 800 | 2.37E−09 | 1.69E−09 |
| 801 | 1.04E−07 | 4.05E−08 |
| 802 | 4.76E−08 | 1.25E−08 |
| 803 | n.d. | n.d. |
| 804 | 6.42E−09 | 3.00E−09 |
| 805 | 1.90E−07 | 7.04E−08 |
| 806 | 3.49E−09 | 1.14E−09 |
| 807 | 1.10E−07 | 6.38E−08 |
| 808 | 8.12E−10 | 1.16E−09 |
| 809 | 1.82E−08 | 9.05E−09 |
| 810 | n.d. | 7.71E−07 |
| 811 | >5.71E−6 | 4.33E−08 |
| | 4.65E−6 | |
| | >2.00E−6 | |
| 812 | 3.57E−09 | 2.47E−09 |
| 813 | >2.00E−6 | 1.92E−6 |
| | | >2.00E−6 |
| | | >2.00E−6 |
| | | >2.00E−6 |
| 814 | 2.50E−08 | 1.10E−08 |
| 815 | 2.78E−10 | 1.60E−09 |
| 816 | 1.05E−09 | 9.90E−10 |
| 817 | 5.24E−10 | 1.94E−09 |
| 818 | 6.20E−10 | 2.18E−09 |
| 819 | 2.31E−10 | 3.85E−10 |
| 820 | 6.33E−09 | 7.15E−08 |
| 821 | 2.98E−10 | 7.39E−10 |
| 822 | 7.23E−10 | 1.68E−09 |
| 823 | 2.13E−09 | 3.01E−09 |
| 824 | 9.98E−10 | 8.45E−10 |
| 825 | 3.72E−10 | 4.75E−10 |
| 826 | 1.77E−09 | 2.26E−09 |
| 827 | n.d. | 4.09E−10 |
| 828 | 1.78E−07 | 1.35E−05 |
| 829 | n.d. | 4.68E−08 |
| 830 | n.d. | >2.00E−5 |
| 831 | 8.88E−10 | 1.54E−09 |
| 832 | 2.58E−09 | 5.68E−09 |
| 833 | 1.12E−09 | 1.27E−09 |
| 834 | 6.25E−09 | 1.07E−08 |
| 835 | 1.54E−08 | 1.85E−08 |
| 836 | 4.19E−09 | 4.95E−09 |
| 837 | 7.60E−10 | 1.81E−09 |
| 838 | 9.63E−10 | 2.02E−09 |
| 839 | n.d. | n.d. |
| 840 | n.d. | n.d. |
| 841 | 1.82E−09 | 3.01E−09 |
| 842 | 4.03E−10 | 9.92E−09 |
| 843 | 4.46E−09 | 1.11E−08 |
| 844 | 1.76E−09 | 1.09E−08 |
| 845 | 6.71E−08 | 2.47E−07 |
| 846 | 1.29E−08 | 2.32E−07 |
| 847 | 3.02E−09 | 6.01E−08 |
| 848 | 1.69E−07 | 1.97E−07 |
| 849 | 4.87E−07 | 5.13E−07 |
| 850 | n.d. | n.d. |
| 851 | 9.17E−10 | 1.76E−09 |
| 852 | 8.28E−09 | 6.62E−08 |
| 853 | 2.04E−09 | 6.86E−08 |
| 854 | 7.05E−09 | 1.47E−07 |
| 855 | 2.28E−09 | 2.61E−08 |
| 856 | >2.00E−5 | 1.96E−5 |
| | | >2.00E−5 |
| 857 | 4.25E−06 | 2.14E−06 |
| 858 | 1.07E−07 | 1.56E−06 |
| 859 | 4.11E−09 | 3.94E−08 |
| 860 | 9.26E−10 | 1.60E−08 |
| 861 | 1.34E−07 | n.d. |
| 862 | 1.86E−09 | 4.12E−09 |
| 863 | 2.98E−09 | 1.29E−08 |
| 864 | 2.57E−08 | 3.78E−07 |
| 865 | 4.02E−09 | 1.18E−08 |
| 866 | 5.75E−08 | 1.44E−07 |
| 867 | 2.60E−09 | 3.00E−09 |
| 868 | 6.28E−09 | 6.29E−09 |
| 869 | >2.00E−5 | n.d. |
| 870 | 1.09E−08 | 1.07E−08 |
| 871 | n.d. | n.d. |
| 872 | 6.57E−09 | 1.58E−08 |
| 873 | n.d. | n.d. |
| 874 | 2.31E−08 | 6.17E−08 |
| 875 | 1.69E−08 | 3.15E−08 |
| 876 | 5.39E−08 | 1.10E−07 |
| 877 | 8.72E−08 | 1.60E−07 |
| 878 | 2.78E−08 | 4.33E−08 |
| 879 | 1.92E−07 | 2.72E−07 |
| 880 | 5.77E−08 | 1.93E−07 |
| 881 | 6.04E−09 | 1.22E−08 |
| 882 | 5.91E−09 | 5.13E−09 |
| 883 | 5.84E−09 | 7.79E−09 |
| 884 | 6.66E−09 | 8.31E−09 |
| 885 | 1.24E−09 | 2.94E−09 |
| 886 | 6.83E−08 | 9.93E−08 |
| 887 | 1.37E−09 | 1.01E−08 |
| 888 | 1.73E−08 | 2.48E−08 |
| 889 | 3.90E−09 | 4.45E−09 |
| 890 | 4.05E−09 | 9.30E−09 |
| 891 | 5.75E−08 | 6.68E−08 |
| 893 | 2.65E−08 | 1.82E−08 |
| 894 | 2.01E−09 | 1.33E−09 |
| 895 | 1.18E−08 | 1.73E−08 |
| 896 | 2.72E−07 | 1.33E−07 |
| 897 | 4.02E−08 | 2.85E−08 |
| 898 | 1.20E−07 | 2.52E−08 |
| 899 | 1.12E−09 | 8.85E−10 |
| 900 | 4.40E−08 | 7.23E−08 |
| 901 | 2.88E−09 | 4.79E−09 |
| 902 | 8.00E−08 | 9.50E−08 |
| 903 | 4.09E−09 | 6.59E−09 |
| 904 | 1.13E−09 | 1.20E−09 |
| 905 | 6.06E−08 | 4.72E−08 |
| 906 | 2.30E−08 | 1.95E−08 |
| 907 | 9.93E−10 | 1.39E−09 |
| 908 | 2.87E−08 | 1.88E−08 |
| 909 | 2.36E−09 | 1.68E−09 |
| 910 | 2.40E−08 | 2.47E−08 |
| 911 | 1.57E−09 | 1.98E−09 |
| 912 | 2.16E−08 | 1.37E−08 |
| 913 | 1.32E−09 | 1.71E−09 |
| 914 | 8.96E−10 | 1.36E−09 |
| 915 | 1.74E−08 | 1.87E−08 |
| 916 | 1.06E−09 | 2.22E−09 |
| 917 | 3.76E−08 | 6.56E−08 |
| 918 | 3.07E−08 | 3.19E−08 |
| 919 | 1.18E−06 | 6.13E−07 |
| 920 | 7.45E−09 | 1.10E−08 |
| 921 | 4.24E−08 | 9.83E−08 |
| 922 | 2.47E−09 | 2.03E−09 |
| 923 | 3.26E−09 | 2.13E−09 |
| 924 | 6.71E−09 | 3.80E−09 |
| 925 | 6.08E−09 | 4.14E−09 |
| 926 | 3.45E−08 | 6.16E−09 |
| 927 | 6.50E−08 | 3.44E−08 |
| 928 | 1.81E−08 | 5.76E−09 |
| 929 | 3.93E−08 | 1.09E−08 |
| 930 | 5.21E−06 | 1.41E−05 |
| 931 | 1.30E−07 | 2.97E−07 |
| 932 | 1.48E−07 | 1.73E−07 |
| 933 | 7.39E−08 | 1.38E−07 |
| 934 | 2.39E−07 | 1.16E−07 |
| 935 | 4.72E−06 | 1.12E−06 |
| 936 | 1.27E−08 | 6.69E−08 |
| 937 | >2.00E−5 | >2.00E−5 |
| 938 | 4.23E−07 | 5.55E−06 |
| 939 | 1.19E−08 | 2.25E−08 |
| 940 | 1.79E−08 | 5.70E−08 |
| 941 | 3.61E−08 | 6.75E−08 |
| 942 | 7.67E−07 | 4.75E−06 |
| 943 | 9.47E−07 | 2.79E−06 |
| 944 | 1.67E−06 | 4.62E−06 |
| 945 | 4.79E−07 | 2.08E−06 |
| 946 | 8.75E−09 | 2.16E−09 |
| 947 | 5.91E−10 | 3.24E−10 |
| 948 | 2.08E−08 | 4.96E−08 |
| 949 | 4.40E−09 | 1.47E−08 |
| 950 | 1.94E−08 | 3.63E−08 |
| 951 | 3.22E−09 | 5.44E−09 |
| 952 | 3.97E−09 | 6.74E−09 |
| 953 | 2.14E−08 | 1.49E−08 |
| 954 | 7.73E−08 | 3.41E−07 |
| 955 | 5.57E−08 | 4.52E−07 |
| 956 | 5.77E−09 | 8.50E−09 |
| 957 | 1.22E−08 | 1.68E−08 |
| 958 | 1.90E−09 | 2.84E−09 |
| 959 | 5.36E−08 | 3.68E−07 |
| 960 | 4.02E−08 | 9.30E−08 |
| 961 | 7.95E−07 | 2.72E−07 |
| 962 | 4.37E−09 | 1.09E−08 |
| 963 | 1.76E−09 | 4.79E−09 |
| 964 | n.d. | 4.60E−09 |
| 965 | n.d. | 1.20E−08 |
| 966 | 9.02E−09 | 9.29E−09 |
| 967 | >2.00E−5 | >2.00E−5 |
| 968 | 6.97E−09 | 7.46E−09 |
| 969 | 6.30E−07 | 2.98E−07 |
| 970 | n.d. | 5.29E−08 |
| 971 | 7.72E−07 | 2.86E−07 |
| 972 | 3.09E−08 | 7.12E−09 |
| 973 | n.d. | 8.28E−09 |
| 974 | 8.12E−10 | 1.86E−09 |
| 975 | 8.67E−10 | 4.24E−09 |
| 976 | 3.11E−06 | 4.64E−06 |
| 977 | n.d. | 4.10E−10 |
| 978 | 2.03E−09 | 1.34E−09 |
| 979 | 2.13E−08 | 1.05E−08 |
| 980 | 5.22E−09 | 6.35E−09 |
| 981 | 2.69E−09 | 5.95E−09 |
| 982 | 9.42E−09 | 2.06E−08 |
| 983 | 8.18E−08 | 1.89E−07 |
| 984 | 3.84E−08 | 4.12E−08 |
| 985 | 6.49E−08 | 9.50E−08 |
| 986 | n.d. | n.d. |
| 987 | 8.61E−09 | 1.32E−08 |
| 988 | 1.49E−08 | 1.38E−08 |
| 989 | 5.18E−07 | 3.99E−07 |
| 990 | 4.29E−08 | 7.36E−08 |
| 991 | 3.13E−08 | 7.66E−08 |
| 992 | 3.94E−09 | 7.74E−09 |
| 993 | 5.88E−07 | 4.59E−06 |
| 994 | 2.32E−08 | 2.56E−08 |
| 995 | 6.97E−10 | 2.11E−09 |
| 996 | 2.80E−07 | 7.81E−08 |
| 997 | 8.11E−08 | 1.38E−07 |
| 998 | 4.16E−07 | 2.79E−07 |
| 999 | 6.91E−09 | 4.27E−09 |
| 1000 | 9.07E−09 | 8.30E−09 |
| 1001 | 3.54E−07 | 5.04E−06 |
| 1002 | 1.26E−09 | 4.20E−08 |
| 1003 | 9.15E−10 | 4.06E−09 |
| 1004 | 1.90E−08 | 2.66E−07 |

1: A compound of formula (I): 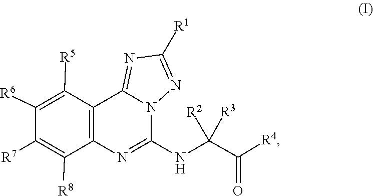
wherein
R¹ is phenyl or heteroaryl,
optionally substituted one to three times, independently from each other, with halogen, cyano, hydroxy, C₁-C₄-alkyl, C₁-C₄-alkoxy, C₁-C₄-haloalkyl, C₁-C₄-haloalkoxy, C₁-C₄-hydroxyalkyl, C₁-C₄-alkoxy-C₁-C₄-alkyl-, C₃-C₆-cycloalkyl, C₃-C₆-cycloalkyl-C₁-C₄-alkyl-, C₃-C₆-cycloalkyl-O—, 4- to 6-membered heterocycloalkyl, —NR⁹R¹⁰, R⁹R¹⁰N—C₁-C₄-alkyl-, C₁-C₃-alkyl-S(O)ₘ— or C₁-C₃-alkyl-SO(NH)—;
R² is hydrogen, C₁-C₄-alkyl, C₁-C₄-haloalkyl or C₃-C₆-cycloalkyl;
R³ is hydrogen, C₁-C₆-alkyl, phenyl or phenyl-C₁-C₃-alkyl, wherein
said C₁-C₆-alkyl group is optionally substituted, one or more times, independently from each other, with hydroxy, halogen, C₁-C₄-alkoxy, —S(O)ₙ—C₁-C₄-alkyl, phenyl-C₁-C₃-alkoxy or —NR⁹R¹⁰ and
said phenyl groups are optionally substituted, one or more times, independently from each other, with hydroxy, halogen, cyano, C₁-C₃-alkyl, C₁-C₃-haloalkyl, C₁-C₃-alkoxy or C₁-C₃-haloalkoxy, or
R² and R³ together with the carbon atom to which they are attached form a 3- to 6-membered ring, said ring optionally containing one heteroatom selected from O, S, NH, NR^{a} in which R^{a} represents a C₁-C₄-alkyl group;
R⁴ is hydroxy, C₁-C₄-alkoxy or —NR¹¹R¹², or
R² and R⁴ together is *—C₂-C₅-alkanediyl-X¹—**, *—C₁-C₂-alkanediyl-X²—C₁-C₃-alkanediyl-** or *—C₁-C₂-alkanediyl-X²—C₂-C₃-alkanediyl-X¹—** to form a 5- to 9-membered ring,
wherein * indicates the point of attachment of said group for R² and ** indicates the point of attachment of said group for R⁴;
R⁵ is hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 4- to 6-membered heterocycloalkyl, —CO₂—C₁-C₄-alkyl, —CO—NR⁹R¹⁰ or —NR⁹R¹⁰;
R⁶ is hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁷ represents is hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl or —NR⁹R¹⁰;
R⁸ is hydrogen, halogen, cyano, hydroxy, C₁-C₄-alkyl, C₁-C₄-alkoxy, C₁-C₄-haloalkyl, C₁-C₄-haloalkoxy, C₃-C₆-cycloalkyl, 1-R¹⁵—C₃-C₆-cycloalkyl, —CO₂—C₁-C₄-alkyl, —CO—NR⁹R¹⁰, —NR⁹R¹⁰, C₁-C₄-hydroxyalkyl, C₁-C₄-alkoxy-C₁-C₄-alkyl-, C₁-C₄-alkyl-S—, C₁-C₄-alkyl-S—C₁-C₄-alkyl-, —S(═O)R′, —S(═O)₂R′, —S(═O)₂NH₂, —S(═O)₂NHR′, —S(═O)₂N(R′)R″, —S(═O)(═NH)R′, 4- to 6-membered heterocycloalkyl, or —OR¹⁶;
R⁹ and R¹⁰ are the same or different and represent, independently from each other, hydrogen, C₁-C₃-alkyl or tert-butoxycarbonyl, or
together with the nitrogen atom to which they are attached form a 4- to 6-membered nitrogen containing heterocyclic ring, said ring optionally containing one additional heteroatom selected from O, S, NH, NR^{a} in which R^{a} represents C₁-C₄-alkyl or C₁-C₄-alkoxycarbonyl;
R¹¹ and R¹² are the same or different and represent, independently from each other, hydrogen, C₁-C₄-alkyl, C₂-C₄-hydroxyalkyl, C₁-C₄-alkoxy-C₂-C₄-alkyl-, R⁹R¹⁰N—C₂-C₄-alkyl-, C₃-C₆-cycloalkyl, 4- to 7-membered heterocycloalkyl, said 4- to 7-membered heterocycloalkyl group is optionally substituted, one or two times, independently from each other, with hydroxy, oxo, halogen, C₁-C₄-alkyl, C₁-C₄-alkoxy, or —NR⁹R¹⁰, or
together with the nitrogen atom to which they are attached form a 4- to 6-membered nitrogen containing heterocyclic ring, said ring optionally containing one additional heteroatom selected from O, S, NH, NR^{a} in which R^{a} represents C₁-C₄-alkyl or C₁-C₄-alkoxycarbonyl and is optionally substituted, one or two times, independently from each other, with hydroxy, halogen, C₁-C₄-alkyl, C₁-C₄-alkoxy, or —NR⁹R¹⁰, or
together with the nitrogen atom to which they are attached form a heterospirocycloalkyl group, which is optionally substituted, one or two times, independently from each other, with hydroxy, halogen, C₁-C₄-alkyl, C₁-C₄-alkoxy, or —NR⁹R¹⁰, or
together with the nitrogen atom to which they are attached form a bridged heterocycloalkyl group, which is optionally substituted, one or two times, independently from each other, with hydroxy, halogen, C₁-C₄-alkyl, C₁-C₄-alkoxy, or —NR⁹R¹⁰;
R¹³ is hydrogen, C₁-C₄-alkyl, benzyl, 4-methoxybenzyl or tert-butoxycarbonyl;
R¹⁴ is hydrogen, C₁-C₄-alkyl, benzyl or 4-methoxybenzyl;
R¹⁵ is C₁-C₃-alkyl or C₁-C₃-haloalkyl;
R¹⁶ is C₂-C₆-hydroxyalkyl, C₁-C₄-alkoxy-C₂-C₆-alkyl-, or C₃-C₆-cycloalkyl;
R′ and R″ are, independently from each other, C₁-C₆-alkyl, C₁-C₆-haloalkyl, or C₃-C₆-cycloalkyl;
X¹ is O, S(O)ₘ, or NR¹³;
X² is O, S(O)ₘ, or NR¹⁴;
m is 0, 1 or 2;
n is 0, 1 or 2;
their polymorphs, enantiomeres, diastereomeres, racemates, tautomeres, N-oxides, hydrates and solvates, as well as their physiological acceptable salts and solvates of these salts, as well as mixtures of the same.

2: The compound according to claim 1, wherein:
R¹ is phenyl or monocyclic heteroaryl,
optionally substituted one to two times, independently from each other, with halogen, hydroxy, C₁-C₄-alkyl, C₁-C₄-alkoxy, C₁-C₄-haloalkyl, C₁-C₄-haloalkoxy, C₃-C₆-cycloalkyl, C₃-C₆-cycloalkyl-C₁-C₄-alkyl-, C₃-C₆-cycloalkyl-O—, 4- to 6-membered heterocycloalkyl or —NR⁹R¹⁰;
R² is hydrogen or C₁-C₄-alkyl;
R³ is hydrogen, C₁-C₄-alkyl, phenyl or phenyl-methyl, wherein
said C₁-C₄-alkyl group is optionally substituted once with hydroxy, methoxy, —S(O)ₙ-methyl, phenyl-methoxy or —NR⁹R¹⁰ and
said phenyl groups are optionally substituted once with hydroxy, or
R² and R³ together with the carbon atom to which they are attached form a 3- to 6-membered ring, said ring optionally containing one oxygen atom;
R⁴ is hydroxy, methoxy or —NR¹¹R¹², or
R² and R⁴ come together along with the atoms between them to form a group selected from: 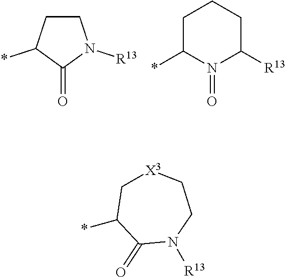
wherein * indicates the point of attachment of said group with the NH group in formula (I);
R⁵ is hydrogen, halogen, C₁-C₄-alkyl, methoxy, trifluoromethyl or cyclopropyl;
R⁶ is hydrogen, halogen or methyl;
R⁷ is hydrogen, halogen, methyl or methoxy;
R⁸ is hydrogen, halogen or methyl;
R⁹ and R¹⁰ are the same or different and represent, independently from each other, hydrogen, methyl or tert-butoxycarbonyl;
R¹¹ and R¹² are the same or different and represent, independently from each other, hydrogen, C₁-C₃-alkyl or C₃-C₄-cycloalkyl, wherein said C₁-C₃-alkyl group is optionally substituted with hydroxy;
R¹³ is hydrogen or methyl;
X³ is CH₂ or NH;
n is 0 or 2;
their polymorphs, enantiomeres, diastereomeres, racemates, tautomeres, N-oxides, hydrates and solvates, as well as their physiological acceptable salts and solvates of these salts, as well as mixtures of the same.

3: The compound according to claim 1, selected from the group consisting of:
(3S)-3-[(2-phenyl[1,2,4]triazolo[1,5-c]quinazolin-5-yl)amino]azepan-2-one;
N²-(2-phenyl[1,2,4]triazolo[1,5-c]quinazolin-5-yl)-D-serinamide;
N²-(2-phenyl[1,2,4]triazolo[1,5-c]quinazolin-5-yl)-D-valinamide;
(2R)-2-[(2-phenyl[1,2,4]triazolo[1,5-c]quinazolin-5-yl)amino]butanamide;
(3R)-3-[(2-phenyl[1,2,4]triazolo[1,5-c]quinazolin-5-yl)amino]azepan-2-one;
(3R)-3-{[2-(4-chlorophenyl)[1,2,4]triazolo[1,5-c]quinazolin-5-yl]amino}azepan-2-one;
(3S)-3-{[2-(4-chlorophenyl)[1,2,4]triazolo[1,5-c]quinazolin-5-yl]amino}azepan-2-one;
(3S)-3-{[2-(3-fluorophenyl)[1,2,4]triazolo[1,5-c]quinazolin-5-yl]amino}piperidin-2-one;
(3S)-3-{[2-(3-fluorophenyl)[1,2,4]triazolo[1,5-c]quinazolin-5-yl]amino}pyrrolidin-2-one;
(3S)-3-{[2-(3-fluorophenyl)[1,2,4]triazolo[1,5-c]quinazolin-5-yl]amino}azepan-2-one;
(3R)-3-{[2-(3-fluorophenyl)[1,2,4]triazolo[1,5-c]quinazolin-5-yl]amino}azepan-2-one;
(3R)-3-{[2-(1-methyl-1H-pyrazol-4-yl)[1,2,4]triazolo[1,5-c]quinazolin-5-yl]amino}azepan-2-one;
3-{[2-(1-methyl-1H-pyrazol-4-yl)[1,2,4]triazolo[1,5-c]quinazolin-5-yl]amino}piperidin-2-one;
(3S)-3-{[2-(1-methyl-1H-pyrazol-4-yl)[1,2,4]triazolo[1,5-c]quinazolin-5-yl]amino}azepan-2-one;
3-{[2-(1-methyl-1H-pyrazol-4-yl)[1,2,4]triazolo[1,5-c]quinazolin-5-yl]amino}pyrrolidin-2-one;
N²-[2-(1-methyl-1H-pyrazol-4-yl)[1,2,4]triazolo[1,5-c]quinazolin-5-yl]-D-serinamide;
(2R)-2-{[2-(1-methyl-1H-pyrazol-4-yl)[1,2,4]triazolo[1,5-c]quinazolin-5-yl]amino}butanamide;
(3R)-3-{[2-(1-methyl-1H-pyrazol-4-yl)[1,2,4]triazolo[1,5-c]quinazolin-5-yl]amino}pyrrolidin-2-one;
(3S)-3-{[2-(1-methyl-1H-pyrazol-4-yl)[1,2,4]triazolo[1,5-c]quinazolin-5-yl]amino}pyrrolidin-2-one;
(3R)-3-{[2-(1-methyl-1H-pyrazol-4-yl)[1,2,4]triazolo[1,5-c]quinazolin-5-yl]amino}piperidin-2-one;
(3S)-3-{[2-(1-methyl-1H-pyrazol-4-yl)[1,2,4]triazolo[1,5-c]quinazolin-5-yl]amino}piperidin-2-one;
6-{[2-(1-methyl-1H-pyrazol-4-yl)[1,2,4]triazolo[1,5-c]quinazolin-5-yl]amino}-1,4-diazepan-5-one;
(6R)-6-{[2-(1-methyl-1H-pyrazol-4-yl)[1,2,4]triazolo[1,5-c]quinazolin-5-yl]amino}-1,4-diazepan-5-one;
(6S)-6-{[2-(1-methyl-1H-pyrazol-4-yl)[1,2,4]triazolo[1,5-c]quinazolin-5-yl]amino}-1,4-diazepan-5-one;
tert-butyl [(5S)-6-amino-5-{[2-(4-methoxyphenyl)[1,2,4]triazolo[1,5-c]quinazolin-5-yl]amino}-6-oxohexyl]carbamate;
N²-[2-(4-methoxyphenyl)[1,2,4]triazolo[1,5-c]quinazolin-5-yl]-D-serinamide;
N²-[2-(4-methoxyphenyl)[1,2,4]triazolo[1,5-c]quinazolin-5-yl]-D-leucinamide;
3-{[2-(4-methoxyphenyl)[1,2,4]triazolo[1,5-c]quinazolin-5-yl]amino}piperidin-2-one;
(3R)-3-{[2-(4-methoxyphenyl)[1,2,4]triazolo[1,5-c]quinazolin-5-yl]amino}azepan-2-one;
(3S)-3-{[2-(4-methoxyphenyl)[1,2,4]triazolo[1,5-c]quinazolin-5-yl]amino}azepan-2-one;
3-{[2-(4-methoxyphenyl)[1,2,4]triazolo[1,5-c]quinazolin-5-yl]amino}pyrrolidin-2-one;
(3R)-3-{[2-(4-methoxyphenyl)[1,2,4]triazolo[1,5-c]quinazolin-5-yl]amino}azepan-2-one;
N²-[2-(4-methoxyphenyl)[1,2,4]triazolo[1,5-c]quinazolin-5-yl]-L-lysinamide;
6-{[2-(4-methoxyphenyl)[1,2,4]triazolo[1,5-c]quinazolin-5-yl]amino}-1,4-diazepan-5-one;
(6S)-6-{[2-(4-methoxyphenyl)[1,2,4]triazolo[1,5-c]quinazolin-5-yl]amino}-1,4-diazepan-5-one;
(6R)-6-{[2-(4-methoxyphenyl)[1,2,4]triazolo[1,5-c]quinazolin-5-yl]amino}-1,4-diazepan-5-one;
(3R)-3-{[2-(4-methoxyphenyl)[1,2,4]triazolo[1,5-c]quinazolin-5-yl]amino}piperidin-2-one;
(3S)-3-{[2-(4-methoxyphenyl)[1,2,4]triazolo[1,5-c]quinazolin-5-yl]amino}piperidin-2-one;
(3R)-3-{[2-(4-methoxyphenyl)[1,2,4]triazolo[1,5-c]quinazolin-5-yl]amino}pyrrolidin-2-one;
(3S)-3-{[2-(4-methoxyphenyl)[1,2,4]triazolo[1,5-c]quinazolin-5-yl]amino}pyrrolidin-2-one;
(3S)-3-{[2-(2-methylphenyl)[1,2,4]triazolo[1,5-c]quinazolin-5-yl]amino}azepan-2-one;
(3R)-3-{[2-(2-methylphenyl)[1,2,4]triazolo[1,5-c]quinazolin-5-yl]amino}azepan-2-one;
3-{[2-(2-methylphenyl)[1,2,4]triazolo[1,5-c]quinazolin-5-yl]amino}pyrrolidin-2-one;
3-{[2-(2-methylphenyl)[1,2,4]triazolo[1,5-c]quinazolin-5-yl]amino}piperidin-2-one;
(6R)-6-{[2-(2-methylphenyl)[1,2,4]triazolo[1,5-c]quinazolin-5-yl]amino}-1,4-diazepan-5-one;
(6R)-6-{[2-(2-methylphenyl)[1,2,4]triazolo[1,5-c]quinazolin-5-yl]amino}-1,4-diazepan-5-one;
(6S)-6-{[2-(2-methylphenyl)[1,2,4]triazolo[1,5-c]quinazolin-5-yl]amino}-1,4-diazepan-5-one;
(3R)-3-{[2-(2-methylphenyl)[1,2,4]triazolo[1,5-c]quinazolin-5-yl]amino}piperidin-2-one;
(3S)-3-{[2-(2-methylphenyl)[1,2,4]triazolo[1,5-c]quinazolin-5-yl]amino}piperidin-2-one;
(3R)-3-{[2-(2-methylphenyl)[1,2,4]triazolo[1,5-c]quinazolin-5-yl]amino}pyrrolidin-2-one;
(3S)-3-{[2-(2-methylphenyl)[1,2,4]triazolo[1,5-c]quinazolin-5-yl]amino}pyrrolidin-2-one;
(3R)-3-({2-[2-(trifluoromethyl)phenyl][1,2,4]triazolo[1,5-c]quinazolin-5-yl}amino)azepan-2-one;
(3R)-3-{[2-(3-methylphenyl)[1,2,4]triazolo[1,5-c]quinazolin-5-yl]amino}azepan-2-one;
(3R)-3-({2-[3-(trifluoromethyl)phenyl][1,2,4]triazolo[1,5-c]quinazolin-5-yl}amino)azepan-2-one;
(3R)-3-{[2-(2-fluorophenyl)[1,2,4]triazolo[1,5-c]quinazolin-5-yl]amino}azepan-2-one;
(3R)-3-{[2-(4-methylphenyl)[1,2,4]triazolo[1,5-c]quinazolin-5-yl]amino}azepan-2-one;
(3R)-3-{2-[4-(trifluoromethyl)phenyl][1,2,4]triazolo[1,5-c]quinazolin-5-yl}amino)azepan-2-one;
(3R)-3-{[2-(2-chlorophenyl)[1,2,4]triazolo[1,5-c]quinazolin-5-yl]amino}azepan-2-one;
(3R)-3-{[2-(3-chlorophenyl)[1,2,4]triazolo[1,5-c]quinazolin-5-yl]amino}azepan-2-one;
(3S)-3-{[2-(2-methoxyphenyl)[1,2,4]triazolo[1,5-c]quinazolin-5-yl]amino}azepan-2-one;
(3R)-3-{[2-(2-methoxyphenyl)[1,2,4]triazolo[1,5-c]quinazolin-5-yl]amino}azepan-2-one;
(3R)-3-{[2-(1H-pyrazol-3-yl)[1,2,4]triazolo[1,5-c]quinazolin-5-yl]amino}azepan-2-one;
(3R)-3-{[2-(1-methyl-1H-pyrazol-3-yl)[1,2,4]triazolo[1,5-c]quinazolin-5-yl]amino}azepan-2-one;
(3R)-3-{[2-(5-methyl-1H-pyrazol-3-yl)[1,2,4]triazolo[1,5-c]quinazolin-5-yl]amino}azepan-2-one;
(3R)-3-{[2-(1-methyl-1H-pyrazol-5-yl)[1,2,4]triazolo[1,5-c]quinazolin-5-yl]amino}azepan-2-one;
(3R)-3-{[2-(1-ethyl-3-methyl-1H-pyrazol-4-yl)[1,2,4]triazolo[1,5-c]quinazolin-5-yl]amino}azepan-2-one;
(3R)-3-{[2-(1-ethyl-1H-pyrazol-4-yl)[1,2,4]triazolo[1,5-c]quinazolin-5-yl]amino}azepan-2-one;
(3R)-3-{[2-(1,5-dimethyl-1H-pyrazol-4-yl)[1,2,4]triazolo[1,5-c]quinazolin-5-yl]amino}azepan-2-one;
(3R)-3-{[2-(1H-pyrazol-4-yl)[1,2,4]triazolo[1,5-c]quinazolin-5-yl]amino}azepan-2-one;
(3R)-3-{[2-(2-methyl-1,3-oxazol-4-yl)[1,2,4]triazolo[1,5-c]quinazolin-5-yl]amino}azepan-2-one;
(3S)-3-{[2-(4-fluorophenyl)[1,2,4]triazolo[1,5-c]quinazolin-5-yl]amino}azepan-2-one;
(3S)-3-{[2-(4-fluorophenyl)[1,2,4]triazolo[1,5-c]quinazolin-5-yl]amino}piperidin-2-one;
(3S)-3-{[2-(4-fluorophenyl)[1,2,4]triazolo[1,5-c]quinazolin-5-yl]amino}pyrrolidin-2-one;
(3R)-3-{[2-(4-fluorophenyl)[1,2,4]triazolo[1,5-c]quinazolin-5-yl]amino}azepan-2-one;
(3R)-3-{[2-(3-methoxyphenyl)[1,2,4]triazolo[1,5-c]quinazolin-5-yl]amino}azepan-2-one;
(3S)-3-{[2-(3-methoxyphenyl)[1,2,4]triazolo[1,5-c]quinazolin-5-yl]amino}azepan-2-one;
3-{[2-(3-methoxyphenyl)[1,2,4]triazolo[1,5-c]quinazolin-5-yl]amino}piperidin-2-one;
3-{[2-(3-methoxyphenyl)[1,2,4]triazolo[1,5-c]quinazolin-5-yl]amino}pyrrolidin-2-one;
N²-[2-(3-methoxyphenyl)[1,2,4]triazolo[1,5-c]quinazolin-5-yl]-D-serinamide;
3-{[2-(3-methoxyphenyl)[1,2,4]triazolo[1,5-c]quinazolin-5-yl]amino}-1-methylpyrrolidin-2-one;
N²-[2-(3-methoxyphenyl)[1,2,4]triazolo[1,5-c]quinazolin-5-yl]-L-alaninamide;
N²-[2-(3-methoxyphenyl)[1,2,4]triazolo[1,5-c]quinazolin-5-yl]-D-leucinamide;
N²-[2-(3-methoxyphenyl)[1,2,4]triazolo[1,5-c]quinazolin-5-yl]-L-valinamide;
N²-[2-(3-methoxyphenyl)[1,2,4]triazolo[1,5-c]quinazolin-5-yl]-L-tyrosinamide;
N²-[2-(3-methoxyphenyl)[1,2,4]triazolo[1,5-c]quinazolin-5-yl]-L-serinamide;
N²-[2-(3-methoxyphenyl)[1,2,4]triazolo[1,5-c]quinazolin-5-yl]alaninamide;
3-{[2-(3-methoxyphenyl)[1,2,4]triazolo[1,5-c]quinazolin-5-yl]amino}oxetane-3-carboxamide;
(2R)-2-{[2-(3-methoxyphenyl)[1,2,4]triazolo[1,5-c]quinazolin-5-yl]amino}butanamide;
(2R)-2-{[2-(3-methoxyphenyl)[1,2,4]triazolo[1,5-c]quinazolin-5-yl]amino}-2-phenylacetamide;
N²-[2-(3-methoxyphenyl)[1,2,4]triazolo[1,5-c]quinazolin-5-yl]-D-phenylalaninamide;
N-[2-(3-methoxyphenyl)[1,2,4]triazolo[1,5-c]quinazolin-5-yl]-D-serine;
N²-[2-(3-methoxyphenyl)[1,2,4]triazolo[1,5-c]quinazolin-5-yl]-D-alaninamide;
N-[2-(3-methoxyphenyl)[1,2,4]triazolo[1,5-c]quinazolin-5-yl]-D-leucine;
N-[2-(3-methoxyphenyl)[1,2,4]triazolo[1,5-c]quinazolin-5-yl]-D-valine;
(2R)-2-{[2-(3-methoxyphenyl)[1,2,4]triazolo[1,5-c]quinazolin-5-yl]amino}butanoic acid;
N²-[2-(3-methoxyphenyl)[1,2,4]triazolo[1,5-c]quinazolin-5-yl]-D-methioninamide;
O-benzyl-N²-[2-(3-methoxyphenyl)[1,2,4]triazolo[1,5-c]quinazolin-5-yl]-D-threoninamide;
N²-[2-(3-methoxyphenyl)[1,2,4]triazolo[1,5-c]quinazolin-5-yl]-2-methylalaninamide;
1-{[2-(3-methoxyphenyl)[1,2,4]triazolo[1,5-c]quinazolin-5-yl]amino}cyclopentane-1-carboxamide;
1-{[2-(3-methoxyphenyl)[1,2,4]triazolo[1,5-c]quinazolin-5-yl]amino}cyclohexane-1-carboxamide;
tert-butyl [(5S)-6-amino-5-{[2-(3-methoxyphenyl)[1,2,4]triazolo[1,5-c]quinazolin-5-yl]amino}-6-oxohexyl]carbamate;
N²-[2-(3-methoxyphenyl)[1,2,4]triazolo[1,5-c]quinazolin-5-yl]-N-methyl-L-alaninamide;
N²-[2-(3-methoxyphenyl)[1,2,4]triazolo[1,5-c]quinazolin-5-yl]-D-valinamide;
methyl N-[2-(3-methoxyphenyl)[1,2,4]triazolo[1,5-c]quinazolin-5-yl]-D-valinate;
N²-[2-(3-methoxyphenyl)[1,2,4]triazolo[1,5-c]quinazolin-5-yl]-N-propan-2-yl-D-alaninamide;
N-cyclopropyl-N²-[2-(3-methoxyphenyl)[1,2,4]triazolo[1,5-c]quinazolin-5-yl]-D-alaninamide;
N-ethyl-N²-[2-(3-methoxyphenyl)[1,2,4]triazolo[1,5-c]quinazolin-5-yl]-D-alaninamide;
N²-[2-(3-methoxyphenyl)[1,2,4]triazolo[1,5-c]quinazolin-5-yl]-N-methyl-D-alaninamide;
N-cyclobutyl-N²-[2-(3-methoxyphenyl)[1,2,4]triazolo[1,5-c]quinazolin-5-yl]-D-alaninamide;
N²-[2-(3-methoxyphenyl)[1,2,4]triazolo[1,5-c]quinazolin-5-yl]-N,N-dimethyl-D-alaninamide;
N-(2-hydroxyethyl)-N²-[2-(3-methoxyphenyl)[1,2,4]triazolo[1,5-c]quinazolin-5-yl]-D-alaninamide;
N-(3-hydroxypropyl)-N²-[2-(3-methoxyphenyl)[1,2,4]triazolo[1,5-c]quinazolin-5-yl]-D-alaninamide;
(2R)-4-(methanesulfonyl)-2-{[2-(3-methoxyphenyl)[1,2,4]triazolo[1,5-c]quinazolin-5-yl]amino}butanamide;
(2R)-2-{[2-(3-methoxyphenyl)[1,2,4]triazolo[1,5-c]quinazolin-5-yl]amino}-4-(methylsulfonyl)butanamide;
N²-[2-(3-methoxyphenyl)[1,2,4]triazolo[1,5-c]quinazolin-5-yl]-L-lysinamide;
6-{[2-(3-methoxyphenyl)[1,2,4]triazolo[1,5-c]quinazolin-5-yl]amino}-1,4-diazepan-5-one;
(6R)-6-{[2-(3-methoxyphenyl)[1,2,4]triazolo[1,5-c]quinazolin-5-yl]amino}-1,4-diazepan-5-one;
(6S)-6-{[2-(3-methoxyphenyl)[1,2,4]triazolo[1,5-c]quinazolin-5-yl]amino}-1,4-diazepan-5-one;
(3R)-3-{[2-(3-methoxyphenyl)[1,2,4]triazolo[1,5-c]quinazolin-5-yl]amino}piperidin-2-one;
(3S)-3-{[2-(3-methoxyphenyl)[1,2,4]triazolo[1,5-c]quinazolin-5-yl]amino}piperidin-2-one;
(3S)-3-{[2-(3-methoxyphenyl)[1,2,4]triazolo[1,5-c]quinazolin-5-yl]amino}pyrrolidin-2-one;
(3R)-3-{[2-(3-methoxyphenyl)[1,2,4]triazolo[1,5-c]quinazolin-5-yl]amino}pyrrolidin-2-one;
(3S)-3-{[2-(3-methoxyphenyl)[1,2,4]triazolo[1,5-c]quinazolin-5-yl]amino}-1-methylpyrrolidin-2-one;
(3R)-3-{[2-(3-methoxyphenyl)[1,2,4]triazolo[1,5-c]quinazolin-5-yl]amino}-1-methylpyrrolidin-2-one;
(3R)-3-{[2-(pyridin-2-yl)[1,2,4]triazolo[1,5-c]quinazolin-5-yl]amino}azepan-2-one;
(3R)-3-{[2-(pyridin-3-yl)[1,2,4]triazolo[1,5-c]quinazolin-5-yl]amino}azepan-2-one;
(3R)-3-{[2-(pyridin-4-yl)[1,2,4]triazolo[1,5-c]quinazolin-5-yl]amino}azepan-2-one;
(3R)-3-{[2-(pyridazin-4-yl)[1,2,4]triazolo[1,5-c]quinazolin-5-yl]amino}azepan-2-one;
(3R)-3-{2-[4-(dimethylamino)phenyl][1,2,4]triazolo[1,5-c]quinazolin-5-yl}amino)azepan-2-one;
(3R)-3-{[2-(3-hydroxyphenyl)[1,2,4]triazolo[1,5-c]quinazolin-5-yl]amino}azepan-2-one;
(3R)-3-{[2-(furan-2-yl)[1,2,4]triazolo[1,5-c]quinazolin-5-yl]amino}azepan-2-one;
(3R)-3-{[2-(3-methyl-1,2,4-oxadiazol-5-yl)[1,2,4]triazolo[1,5-c]quinazolin-5-yl]amino}azepan-2-one;
(3R)-3-{[2-(3-ethyl-1,2,4-oxadiazol-5-yl)[1,2,4]triazolo[1,5-c]quinazolin-5-yl]amino}azepan-2-one;
(3R)-3-({2-[3-(propan-2-yl)-1,2,4-oxadiazol-5-yl][1,2,4]triazolo[1,5-c]quinazolin-5-yl}amino)azepan-2-one;
(3R)-3-{[2-(3-tert-butyl-1,2,4-oxadiazol-5-yl)[1,2,4]triazolo[1,5-c]quinazolin-5-yl]amino}azepan-2-one;
(3R)-3-{[2-(3-cyclopropyl-1,2,4-oxadiazol-5-yl)[1,2,4]triazolo[1,5-c]quinazolin-5-yl]amino}azepan-2-one;
(3R)-3-{2-[3-(trifluoromethyl)-1,2,4-oxadiazol-5-yl][1,2,4]triazolo[1,5-c]quinazolin-5-yl}amino)azepan-2-one;
(3R)-3-{[7-fluoro-2-(3-fluorophenyl)[1,2,4]triazolo[1,5-c]quinazolin-5-yl]amino}azepan-2-one;
(3R)-3-{[7-fluoro-2-(4-methoxyphenyl)[1,2,4]triazolo[1,5-c]quinazolin-5-yl]amino}azepan-2-one;
(3R)-3-{[7-fluoro-2-(4-fluorophenyl)[1,2,4]triazolo[1,5-c]quinazolin-5-yl]amino}azepan-2-one;
(3R)-3-{[2-(3-methoxyphenyl)-7-methyl[1,2,4]triazolo[1,5-c]quinazolin-5-yl]amino}azepan-2-one;
(3S)-3-{[2-(3-methoxyphenyl)-7-methyl[1,2,4]triazolo[1,5-c]quinazolin-5-yl]amino}azepan-2-one;
(3R)-3-{[7-methyl-2-(1-methyl-1H-pyrazol-4-yl)[1,2,4]triazolo[1,5-c]quinazolin-5-yl]amino}azepan-2-one;
(3R)-3-{[2-(4-methoxyphenyl)-7-methyl[1,2,4]triazolo[1,5-c]quinazolin-5-yl]amino}azepan-2-one;
(3S)-3-{[2-(4-methoxyphenyl)-7-methyl[1,2,4]triazolo[1,5-c]quinazolin-5-yl]amino}azepan-2-one;
(3R)-3-{[2-(3-methoxyphenyl)-8-methyl[1,2,4]triazolo[1,5-c]quinazolin-5-yl]amino}azepan-2-one;
(3S)-3-{[2-(3-methoxyphenyl)-8-methyl[1,2,4]triazolo[1,5-c]quinazolin-5-yl]amino}azepan-2-one;
(3R)-3-{[8-methyl-2-(1-methyl-1H-pyrazol-4-yl)[1,2,4]triazolo[1,5-c]quinazolin-5-yl]amino}azepan-2-one;
(3R)-3-{[8-fluoro-2-(3-methoxyphenyl)[1,2,4]triazolo[1,5-c]quinazolin-5-yl]amino}azepan-2-one;
(3S)-3-{[8-fluoro-2-(3-methoxyphenyl)[1,2,4]triazolo[1,5-c]quinazolin-5-yl]amino}pyrrolidin-2-one;
(3S)-3-{[8-fluoro-2-(3-methoxyphenyl)[1,2,4]triazolo[1,5-c]quinazolin-5-yl]amino}azepan-2-one;
(3S)-3-{[8-fluoro-2-(3-methoxyphenyl)[1,2,4]triazolo[1,5-c]quinazolin-5-yl]amino}piperidin-2-one;
(3R)-3-{[8-fluoro-2-(3-methoxyphenyl)[1,2,4]triazolo[1,5-c]quinazolin-5-yl]amino}pyrrolidin-2-one;
(3R)-3-{[8-fluoro-2-(4-methoxyphenyl)[1,2,4]triazolo[1,5-c]quinazolin-5-yl]amino}azepan-2-one;
(3R)-3-{[2-(3-methoxyphenyl)-9-methyl[1,2,4]triazolo[1,5-c]quinazolin-5-yl]amino}azepan-2-one;
(3S)-3-{[2-(3-methoxyphenyl)-9-methyl[1,2,4]triazolo[1,5-c]quinazolin-5-yl]amino}pyrrolidin-2-one;
(3S)-3-{[2-(3-methoxyphenyl)-9-methyl[1,2,4]triazolo[1,5-c]quinazolin-5-yl]amino}azepan-2-one;
(3S)-3-{[2-(3-methoxyphenyl)-9-methyl[1,2,4]triazolo[1,5-c]quinazolin-5-yl]amino}piperidin-2-one;
(3R)-3-{[9-methyl-2-(1-methyl-1H-pyrazol-4-yl)[1,2,4]triazolo[1,5-c]quinazolin-5-yl]amino}azepan-2-one;
(3R)-3-{[9-fluoro-2-(3-methoxyphenyl)[1,2,4]triazolo[1,5-c]quinazolin-5-yl]amino}azepan-2-one;
(3S)-3-{[9-fluoro-2-(3-methoxyphenyl)[1,2,4]triazolo[1,5-c]quinazolin-5-yl]amino}azepan-2-one;
3-{[9-fluoro-2-(3-methoxyphenyl)[1,2,4]triazolo[1,5-c]quinazolin-5-yl]amino}piperidin-2-one;
3-{[9-fluoro-2-(3-methoxyphenyl)[1,2,4]triazolo[1,5-c]quinazolin-5-yl]amino}pyrrolidin-2-one;
(3R)-3-{[10-bromo-2-(3-fluorophenyl)[1,2,4]triazolo[1,5-c]quinazolin-5-yl]amino}azepan-2-one;
(3R)-3-{[10-bromo-2-(4-methoxyphenyl)[1,2,4]triazolo[1,5-c]quinazolin-5-yl]amino}azepan-2-one;
(3R)-3-{[10-bromo-2-(1-methyl-1H-pyrazol-4-yl)[1,2,4]triazolo[1,5-c]quinazolin-5-yl]amino}azepan-2-one;
(3R)-3-{[10-bromo-2-(4-fluorophenyl)[1,2,4]triazolo[1,5-c]quinazolin-5-yl]amino}azepan-2-one;
(3R)-3-{[10-chloro-2-(3-fluorophenyl)[1,2,4]triazolo[1,5-c]quinazolin-5-yl]amino}azepan-2-one;
(3R)-3-{[10-chloro-2-(4-methoxyphenyl)[1,2,4]triazolo[1,5-c]quinazolin-5-yl]amino}azepan-2-one;
(3R)-3-{[10-chloro-2-(1-methyl-1H-pyrazol-4-yl)[1,2,4]triazolo[1,5-c]quinazolin-5-yl]amino}azepan-2-one;
(3R)-3-{[10-chloro-2-(4-fluorophenyl)[1,2,4]triazolo[1,5-c]quinazolin-5-yl]amino}azepan-2-one;
(3R)-3-{[2-(3-fluorophenyl)-10-(trifluoromethyl)[1,2,4]triazolo[1,5-c]quinazolin-5-yl]amino}azepan-2-one;
(3R)-3-{[2-(4-methoxyphenyl)-10-(trifluoromethyl)[1,2,4]triazolo[1,5-c]quinazolin-5-yl]amino}azepan-2-one;
(3R)-3-{[2-(1-methyl-1H-pyrazol-4-yl)-10-(trifluoromethyl)[1,2,4]triazolo[1,5-c]quinazolin-5-yl]amino}azepan-2-one;
(3R)-3-{[2-(4-fluorophenyl)-10-(trifluoromethyl)[1,2,4]triazolo[1,5-c]quinazolin-5-yl]amino}azepan-2-one;
(3R)-3-{[10-cyclopropyl-2-(3-fluorophenyl)[1,2,4]triazolo[1,5-c]quinazolin-5-yl]amino}azepan-2-one;
(3R)-3-{[10-cyclopropyl-2-(4-methoxyphenyl)[1,2,4]triazolo[1,5-c]quinazolin-5-yl]amino}azepan-2-one;
(3R)-3-{[10-cyclopropyl-2-(1-methyl-1H-pyrazol-4-yl)[1,2,4]triazolo[1,5-c]quinazolin-5-yl]amino}azepan-2-one;
(3R)-3-{[2-(3-methoxyphenyl)-10-methyl[1,2,4]triazolo[1,5-c]quinazolin-5-yl]amino}azepan-2-one;
3-{[2-(3-methoxyphenyl)-10-methyl[1,2,4]triazolo[1,5-c]quinazolin-5-yl]amino}pyrrolidin-2-one;
3-{[2-(3-methoxyphenyl)-10-methyl[1,2,4]triazolo[1,5-c]quinazolin-5-yl]amino}piperidin-2-one;
(3S)-3-{[2-(3-methoxyphenyl)-10-methyl[1,2,4]triazolo[1,5-c]quinazolin-5-yl]amino}azepan-2-one;
(3S)-3-{[2-(3-methoxyphenyl)-10-methyl[1,2,4]triazolo[1,5-c]quinazolin-5-yl]amino}piperidin-2-one;
(3R)-3-{[10-methyl-2-(1-methyl-1H-pyrazol-4-yl)[1,2,4]triazolo[1,5-c]quinazolin-5-yl]amino}azepan-2-one;
N²-[10-fluoro-2-(3-methoxyphenyl)[1,2,4]triazolo[1,5-c]quinazolin-5-yl]-D-alaninamide;
(3R)-3-{[10-fluoro-2-(3-methoxyphenyl)[1,2,4]triazolo[1,5-c]quinazolin-5-yl]amino}azepan-2-one;
(3R)-3-{[9-fluoro-2-(3-methoxyphenyl)[1,2,4]triazolo[1,5-c]quinazolin-5-yl]amino}pyrrolidin-2-one;
(3S)-3-{[9-fluoro-2-(3-methoxyphenyl)[1,2,4]triazolo[1,5-c]quinazolin-5-yl]amino}pyrrolidin-2-one;
(3R)-3-{[9-fluoro-2-(3-methoxyphenyl)[1,2,4]triazolo[1,5-c]quinazolin-5-yl]amino}piperidin-2-one;
(3S)-3-{[9-fluoro-2-(3-methoxyphenyl)[1,2,4]triazolo[1,5-c]quinazolin-5-yl]amino}piperidin-2-one;
(3R)-3-{[2-(3-methoxyphenyl)-10-methyl[1,2,4]triazolo[1,5-c]quinazolin-5-yl]amino}pyrrolidin-2-one;
(3S)-3-{[2-(3-methoxyphenyl)-10-methyl[1,2,4]triazolo[1,5-c]quinazolin-5-yl]amino}pyrrolidin-2-one;
6-{[2-(4-methoxyphenyl)-10-methyl[1,2,4]triazolo[1,5-c]quinazolin-5-yl]amino}-1,4-diazepan-5-one;
(3R)-3-{[7-chloro-2-(pyridin-4-yl)[1,2,4]triazolo[1,5-c]quinazolin-5-yl]amino}azepan-2-one;
(3R)-3-{[7-chloro-2-(1-methyl-1H-pyrazol-4-yl)[1,2,4]triazolo[1,5-c]quinazolin-5-yl]amino}azepan-2-one;
(3R)-3-{2-[1-(propan-2-yl)-1H-pyrazol-4-yl][1,2,4]triazolo[1,5-c]quinazolin-5-yl}amino)azepan-2-one;
(3R)-3-{[2-(2,3-dichlorophenyl)[1,2,4]triazolo[1,5-c]quinazolin-5-yl]amino}azepan-2-one;
(3R)-3-{[2-(2,5-difluorophenyl)[1,2,4]triazolo[1,5-c]quinazolin-5-yl]amino}azepan-2-one;
(3R)-3-{[2-(2-ethoxyphenyl)[1,2,4]triazolo[1,5-c]quinazolin-5-yl]amino}azepan-2-one;
(3R)-3-{[2-(5-methyl-1,3,4-oxadiazol-2-yl)[1,2,4]triazolo[1,5-c]quinazolin-5-yl]amino}azepan-2-one;
(3R)-3-{[2-(4-bromo-2-chlorophenyl)[1,2,4]triazolo[1,5-c]quinazolin-5-yl]amino}azepan-2-one;
(3R)-3-{[2-(2,4-difluorophenyl)[1,2,4]triazolo[1,5-c]quinazolin-5-yl]amino}azepan-2-one;
(3R)-3-{2-[4-(methylsulfanyl)phenyl][1,2,4]triazolo[1,5-c]quinazolin-5-yl}amino)azepan-2-one;
(3R)-3-{[2-(4-fluorophenyl)-7-(trifluoromethyl)[1,2,4]triazolo[1,5-c]quinazolin-5-yl]amino}azepan-2-one;
(3R)-3-{[2-(4-methoxyphenyl)-7-(trifluoromethyl)[1,2,4]triazolo[1,5-c]quinazolin-5-yl]amino}azepan-2-one;
(3R)-3-{[7-methoxy-2-(4-methoxyphenyl)[1,2,4]triazolo[1,5-c]quinazolin-5-yl]amino}azepan-2-one;
(3R)-3-{[7-cyclopropyl-2-(4-methoxyphenyl)[1,2,4]triazolo[1,5-c]quinazolin-5-yl]amino}azepan-2-one;
(3R)-3-{[7-cyclopropyl-2-(1-methyl-1H-pyrazol-4-yl)[1,2,4]triazolo[1,5-c]quinazolin-5-yl]amino}azepan-2-one;
(3R)-3-({7-bromo-2-[2-(trifluoromethoxy)phenyl][1,2,4]triazolo[1,5-c]quinazolin-5-yl}amino)azepan-2-one;
(3S)-3-{[7-bromo-2-(3-fluorophenyl)[1,2,4]triazolo[1,5-c]quinazolin-5-yl]amino}azepan-2-one;
(3S)-3-{[7-bromo-2-(4-fluorophenyl)[1,2,4]triazolo[1,5-c]quinazolin-5-yl]amino}azepan-2-one;
(3S)-3-{[7-bromo-2-(4-methoxyphenyl)[1,2,4]triazolo[1,5-c]quinazolin-5-yl]amino}azepan-2-one;
(3R)-3-{[7-bromo-2-(1-methyl-1H-pyrazol-4-yl)[1,2,4]triazolo[1,5-c]quinazolin-5-yl]amino}azepan-2-one;
(3R)-3-{[7-bromo-2-(4-fluorophenyl)[1,2,4]triazolo[1,5-c]quinazolin-5-yl]amino}azepan-2-one;
(3R)-3-{[7-bromo-2-(3-fluorophenyl)[1,2,4]triazolo[1,5-c]quinazolin-5-yl]amino}azepan-2-one;
(3R)-3-{[7-bromo-2-(4-methoxyphenyl)[1,2,4]triazolo[1,5-c]quinazolin-5-yl]amino}azepan-2-one;
(3R)-3-{[7-bromo-2-(pyridin-4-yl)[1,2,4]triazolo[1,5-c]quinazolin-5-yl]amino}azepan-2-one;
(3R)-3-{[7-bromo-2-(4-fluorophenyl)[1,2,4]triazolo[1,5-c]quinazolin-5-yl]amino}pyrrolidin-2-one;
(3R)-3-{[7-bromo-2-(4-methoxyphenyl)[1,2,4]triazolo[1,5-c]quinazolin-5-yl]amino}pyrrolidin-2-one;
(3R)-3-({7-bromo-2-[2-(trifluoromethoxy)phenyl][1,2,4]triazolo[1,5-c]quinazolin-5-yl}amino)pyrrolidin-2-one;
(3R)-3-({7-bromo-2-[2-(trifluoromethoxy)phenyl][1,2,4]triazolo[1,5-c]quinazolin-5-yl}amino)piperidin-2-one;
(3R)-3-{[7-bromo-2-(1-methyl-1H-pyrazol-4-yl)[1,2,4]triazolo[1,5-c]quinazolin-5-yl]amino}piperidin-2-one;
(3R)-3-{[7-bromo-2-(1-methyl-1H-pyrazol-4-yl)[1,2,4]triazolo[1,5-c]quinazolin-5-yl]amino}pyrrolidin-2-one;
(3R)-3-{[7-bromo-2-(4-methoxyphenyl)[1,2,4]triazolo[1,5-c]quinazolin-5-yl]amino}piperidin-2-one;
(3R)-3-{[7-bromo-2-(3-fluorophenyl)[1,2,4]triazolo[1,5-c]quinazolin-5-yl]amino}pyrrolidin-2-one;
(3R)-3-{[7-bromo-2-(3-fluorophenyl)[1,2,4]triazolo[1,5-c]quinazolin-5-yl]amino}piperidin-2-one;
(3R)-3-{[7-bromo-2-(4-fluorophenyl)[1,2,4]triazolo[1,5-c]quinazolin-5-yl]amino}piperidin-2-one;
(3R)-3-{[2-(1-methyl-1H-pyrazol-4-yl)-8-(trifluoromethyl)[1,2,4]triazolo[1,5-c]quinazolin-5-yl]amino}azepan-2-one;
(3R)-3-{[2-(4-methoxyphenyl)-8-methyl[1,2,4]triazolo[1,5-c]quinazolin-5-yl]amino}azepan-2-one;
(3R)-3-{[8-bromo-2-(4-methoxyphenyl)[1,2,4]triazolo[1,5-c]quinazolin-5-yl]amino}azepan-2-one;
(3R)-3-{[8-bromo-2-(1-methyl-1H-pyrazol-4-yl)[1,2,4]triazolo[1,5-c]quinazolin-5-yl]amino}azepan-2-one;
(3R)-3-{[9-fluoro-2-(4-methoxyphenyl)[1,2,4]triazolo[1,5-c]quinazolin-5-yl]amino}azepan-2-one;
(3R)-3-{[9-fluoro-2-(1-methyl-1H-pyrazol-4-yl)[1,2,4]triazolo[1,5-c]quinazolin-5-yl]amino}azepan-2-one;
(3R)-3-{[9-fluoro-2-(4-fluorophenyl)[1,2,4]triazolo[1,5-c]quinazolin-5-yl]amino}azepan-2-one;
(3R)-3-{[9-fluoro-2-(2-fluorophenyl)[1,2,4]triazolo[1,5-c]quinazolin-5-yl]amino}azepan-2-one;
(3R)-3-{[9-chloro-2-(4-methoxyphenyl)[1,2,4]triazolo[1,5-c]quinazolin-5-yl]amino}azepan-2-one;
(3R)-3-{[9-chloro-2-(1-methyl-1H-pyrazol-4-yl)[1,2,4]triazolo[1,5-c]quinazolin-5-yl]amino}azepan-2-one;
(3R)-3-{[9-methoxy-2-(4-methoxyphenyl)[1,2,4]triazolo[1,5-c]quinazolin-5-yl]amino}azepan-2-one;
(3R)-3-{[9-methoxy-2-(1-methyl-1H-pyrazol-4-yl)[1,2,4]triazolo[1,5-c]quinazolin-5-yl]amino}azepan-2-one;
(3R)-3-{[2-(4-fluorophenyl)-9-methoxy[1,2,4]triazolo[1,5-c]quinazolin-5-yl]amino}azepan-2-one;
(3R)-3-{[2-(2-fluorophenyl)-9-methoxy[1,2,4]triazolo[1,5-c]quinazolin-5-yl]amino}azepan-2-one;
(3R)-3-{[2-(4-methoxyphenyl)-9-methyl[1,2,4]triazolo[1,5-c]quinazolin-5-yl]amino}azepan-2-one;
(3R)-3-{[9-bromo-2-(4-methoxyphenyl)[1,2,4]triazolo[1,5-c]quinazolin-5-yl]amino}azepan-2-one;
(3R)-3-{[2-(4-methoxyphenyl)-10-methyl[1,2,4]triazolo[1,5-c]quinazolin-5-yl]amino}azepan-2-one;
(3R)-3-{[10-fluoro-2-(1-methyl-1H-pyrazol-4-yl)[1,2,4]triazolo[1,5-c]quinazolin-5-yl]amino}azepan-2-one;
(3R)-3-{[10-methoxy-2-(4-methoxyphenyl)[1,2,4]triazolo[1,5-c]quinazolin-5-yl]amino}azepan-2-one;
2-(4-methoxyphenyl)-5-{[(3R)-2-oxoazepan-3-yl]amino}[1,2,4]triazolo[1,5-c]quinazoline-10-carbonitrile;
2-(1-methyl-1H-pyrazol-4-yl)-5-{[(3R)-2-oxoazepan-3-yl]amino}[1,2,4]triazolo[1,5-c]quinazoline-10-carbonitrile;
2-(4-fluorophenyl)-5-{[(3R)-2-oxoazepan-3-yl]amino}[1,2,4]triazolo[1,5-c]quinazoline-10-carbonitrile;
2-(3-fluorophenyl)-5-{[(3R)-2-oxoazepan-3-yl]amino}[1,2,4]triazolo[1,5-c]quinazoline-10-carbonitrile;
methyl 2-(4-methoxyphenyl)-5-{[(3R)-2-oxoazepan-3-yl]amino}[1,2,4]triazolo[1,5-c]quinazoline-7-carboxylate;
(3R)-3-{[8-cyclopropyl-2-(4-methoxyphenyl)[1,2,4]triazolo[1,5-c]quinazolin-5-yl]amino}azepan-2-one;
methyl 2-(4-methoxyphenyl)-5-{1[(3R)-2-oxoazepan-3-yl]amino}[1,2,4]triazolo[1,5-c]quinazoline-10-carboxylate;
(6R)-6-{[7-chloro-2-(1-methyl-1H-pyrazol-4-yl)[1,2,4]triazolo[1,5-c]quinazolin-5-yl]amino}-1,4-diazepan-5-one;
(6R)-6-{[2-(2-ethoxyphenyl)[1,2,4]triazolo[1,5-c]quinazolin-5-yl]amino}-1,4-diazepan-5-one;
(6R)-6-{[2-(4-bromo-2-chlorophenyl)[1,2,4]triazolo[1,5-c]quinazolin-5-yl]amino}-1,4-diazepan-5-one;
(6R)-6-({2-[4-(methylsulfanyl)phenyl][1,2,4]triazolo[1,5-c]quinazolin-5-yl}amino)-1,4-diazepan-5-one;
(6R)-6-{[2-(1-methyl-1H-pyrazol-3-yl)[1,2,4]triazolo[1,5-c]quinazolin-5-yl]amino}-1,4-diazepan-5-one;
(6R)-6-{[2-(furan-2-yl)[1,2,4]triazolo[1,5-c]quinazolin-5-yl]amino}-1,4-diazepan-5-one;
(6R)-6-({2-[4-(trifluoromethyl)phenyl][1,2,4]triazolo[1,5-c]quinazolin-5-yl}amino)-1,4-diazepan-5-one;
(6R)-6-{[2-(4-fluorophenyl)[1,2,4]triazolo[1,5-c]quinazolin-5-yl]amino}-1,4-diazepan-5-one;
(6R)-6-{[2-(3-fluorophenyl)[1,2,4]triazolo[1,5-c]quinazolin-5-yl]amino}-1,4-diazepan-5-one;
(6R)-6-({2-[2-(trifluoromethyl)phenyl][1,2,4]triazolo[1,5-c]quinazolin-5-yl}amino)-1,4-diazepan-5-one;
(6R)-6-{[2-(2,3-dichlorophenyl)[1,2,4]triazolo[1,5-c]quinazolin-5-yl]amino}-1,4-diazepan-5-one;
(6R)-6-{[2-(1H-pyrazol-4-yl)[1,2,4]triazolo[1,5-c]quinazolin-5-yl]amino}-1,4-diazepan-5-one;
(6R)-6-{[7-methoxy-2-(4-methoxyphenyl)[1,2,4]triazolo[1,5-c]quinazolin-5-yl]amino}-1,4-diazepan-5-one;
(6R)-6-({7-bromo-2-[2-(trifluoromethoxy)phenyl][1,2,4]triazolo[1,5-c]quinazolin-5-yl}amino)-1,4-diazepan-5-one;
(6R)-6-{[7-bromo-2-(1-methyl-1H-pyrazol-4-yl)[1,2,4]triazolo[1,5-c]quinazolin-5-yl]amino}-1,4-diazepan-5-one;
(6R)-6-{[7-bromo-2-(3-fluorophenyl)[1,2,4]triazolo[1,5-c]quinazolin-5-yl]amino}-1,4-diazepan-5-one;
(6R)-6-{[7-bromo-2-(4-fluorophenyl)[1,2,4]triazolo[1,5-c]quinazolin-5-yl]amino}-1,4-diazepan-5-one;
(6R)-6-{[7-bromo-2-(4-methoxyphenyl)[1,2,4]triazolo[1,5-c]quinazolin-5-yl]amino}-1,4-diazepan-5-one;
(6R)-6-{[7-methyl-2-(1-methyl-1H-pyrazol-4-yl)[1,2,4]triazolo[1,5-c]quinazolin-5-yl]amino}-1,4-diazepan-5-one;
(6R)-6-{[8-methoxy-2-(4-methoxyphenyl)[1,2,4]triazolo[1,5-c]quinazolin-5-yl]amino}-1,4-diazepan-5-one;
(6R)-6-{[2-(4-methoxyphenyl)-8-(trifluoromethyl)[1,2,4]triazolo[1,5-c]quinazolin-5-yl]amino}-1,4-diazepan-5-one;
(6R)-6-{[2-(4-methoxyphenyl)-8-methyl[1,2,4]triazolo[1,5-c]quinazolin-5-yl]amino}-1,4-diazepan-5-one;
(6R)-6-{[8-bromo-2-(4-methoxyphenyl)[1,2,4]triazolo[1,5-c]quinazolin-5-yl]amino}-1,4-diazepan-5-one;
(6R)-6-{[8-fluoro-2-(4-methoxyphenyl)[1,2,4]triazolo[1,5-c]quinazolin-5-yl]amino}-1,4-diazepan-5-one;
(6R)-6-{[9-fluoro-2-(4-methoxyphenyl)[1,2,4]triazolo[1,5-c]quinazolin-5-yl]amino}-1,4-diazepan-5-one;
(6R)-6-{[9-chloro-2-(4-methoxyphenyl)[1,2,4]triazolo[1,5-c]quinazolin-5-yl]amino}-1,4-diazepan-5-one;
(6R)-6-{[9-methoxy-2-(4-methoxyphenyl)[1,2,4]triazolo[1,5-c]quinazolin-5-yl]amino}-1,4-diazepan-5-one;
(6R)-6-{[2-(4-methoxyphenyl)-9-methyl[1,2,4]triazolo[1,5-c]quinazolin-5-yl]amino}-1,4-diazepan-5-one;
(6S)-6-{[10-bromo-2-(4-methoxyphenyl)[1,2,4]triazolo[1,5-c]quinazolin-5-yl]amino}-1,4-diazepan-5-one;
(6S)-6-{[10-bromo-2-(4-fluorophenyl)[1,2,4]triazolo[1,5-c]quinazolin-5-yl]amino}-1,4-diazepan-5-one;
(6S)-6-{[10-bromo-2-(3-fluorophenyl)[1,2,4]triazolo[1,5-c]quinazolin-5-yl]amino}-1,4-diazepan-5-one;
(6S)-6-{[10-chloro-2-(3-fluorophenyl)[1,2,4]triazolo[1,5-c]quinazolin-5-yl]amino}-1,4-diazepan-5-one;
(6R)-6-{[2-(4-methoxyphenyl)-10-(trifluoromethyl)[1,2,4]triazolo[1,5-c]quinazolin-5-yl]amino}-1,4-diazepan-5-one;
(6R)-6-{[2-(1-methyl-1H-pyrazol-4-yl)-10-(trifluoromethyl)[1,2,4]triazolo[1,5-c]quinazolin-5-yl]amino}-1,4-diazepan-5-one;
(6R)-6-{[2-(4-fluorophenyl)-10-(trifluoromethyl)[1,2,4]triazolo[1,5-c]quinazolin-5-yl]amino}-1,4-diazepan-5-one;
(6R)-6-{[2-(3-fluorophenyl)-10-(trifluoromethyl)[1,2,4]triazolo[1,5-c]quinazolin-5-yl]amino}-1,4-diazepan-5-one;
(6R)-6-{[2-(3-methoxyphenyl)-10-methyl[1,2,4]triazolo[1,5-c]quinazolin-5-yl]amino}-1,4-diazepan-5-one;
(6R)-6-{[2-(4-methoxyphenyl)-10-methyl[1,2,4]triazolo[1,5-c]quinazolin-5-yl]amino}-1,4-diazepan-5-one;
(6R)-6-{[10-methoxy-2-(4-methoxyphenyl)[1,2,4]triazolo[1,5-c]quinazolin-5-yl]amino}-1,4-diazepan-5-one;
6-{[10-chloro-2-(4-methoxyphenyl)[1,2,4]triazolo[1,5-c]quinazolin-5-yl]amino}-1,4-diazepan-5-one;
6-{[10-chloro-2-(4-fluorophenyl)[1,2,4]triazolo[1,5-c]quinazolin-5-yl]amino}-1,4-diazepan-5-one;
6-{[10-cyclopropyl-2-(1-methyl-1H-pyrazol-4-yl)[1,2,4]triazolo[1,5-c]quinazolin-5-yl]amino}-1,4-diazepan-5-one;
6-{[10-cyclopropyl-2-(3-fluorophenyl)[1,2,4]triazolo[1,5-c]quinazolin-5-yl]amino}-1,4-diazepan-5-one;
(6R)-6-{[10-chloro-2-(4-methoxyphenyl)[1,2,4]triazolo[1,5-c]quinazolin-5-yl]amino}-1,4-diazepan-5-one;
(6S)-6-{[10-chloro-2-(4-methoxyphenyl)[1,2,4]triazolo[1,5-c]quinazolin-5-yl]amino}-1,4-diazepan-5-one;
(6R)-6-{[10-chloro-2-(4-fluorophenyl)[1,2,4]triazolo[1,5-c]quinazolin-5-yl]amino}-1,4-diazepan-5-one;
(6S)-6-{[10-chloro-2-(4-fluorophenyl)[1,2,4]triazolo[1,5-c]quinazolin-5-yl]amino}-1,4-diazepan-5-one;
(6R)-6-{[10-cyclopropyl-2-(3-fluorophenyl)[1,2,4]triazolo[1,5-c]quinazolin-5-yl]amino}-1,4-diazepan-5-one;
(6S)-6-{[10-cyclopropyl-2-(3-fluorophenyl)[1,2,4]triazolo[1,5-c]quinazolin-5-yl]amino}-1,4-diazepan-5-one;
(3R)-3-({2-[3-(hydroxymethyl)-1,2,4-oxadiazol-5-yl][1,2,4]triazolo[1,5-c]quinazolin-5-yl}amino)azepan-2-one;
(3R)-3-({2-[3-(methoxymethyl)-1,2,4-oxadiazol-5-yl][1,2,4]triazolo[1,5-c]quinazolin-5-yl}amino)azepan-2-one;
(3R)-3-[(2-{3-[(dimethylamino)methyl]-1,2,4-oxadiazol-5-yl}[1,2,4]triazolo[1,5-c]quinazolin-5-yl)amino]azepan-2-one;
(3R)-3-({2-[3-(2-hydroxyethyl)-1,2,4-oxadiazol-5-yl][1,2,4]triazolo[1,5-c]quinazolin-5-yl}amino)azepan-2-one;
(3R)-3-({2-[4-(S-methylsulfonimidoyl)phenyl][1,2,4]triazolo[1,5-c]quinazolin-5-yl}amino)azepan-2-one;
(3R, R)-3-({2-[4-(S-methylsulfonimidoyl)phenyl][1,2,4]triazolo[1,5-c]quinazolin-5-yl}amino)azepan-2-one;
(3R, S)-3-({2-[4-(S-methylsulfonimidoyl)phenyl][1,2,4]triazolo[1,5-c]quinazolin-5-yl}amino)azepan-2-one;
(3R)-3-({2-[3-(trifluoromethoxy)phenyl][1,2,4]triazolo[1,5-c]quinazolin-5-yl}amino)azepan-2-one;
(3R)-3-({2-[3-(trifluoromethoxy)phenyl][1,2,4]triazolo[1,5-c]quinazolin-5-yl}amino)pyrrolidin-2-one;
(3R)-3-({2-[3-(trifluoromethoxy)phenyl][1,2,4]triazolo[1,5-c]quinazolin-5-yl}amino)piperidin-2-one;
(3R)-1-methyl-3-({2-[3-(trifluoromethoxy)phenyl][1,2,4]triazolo[1,5-c]quinazolin-5-yl}amino)pyrrolidin-2-one;
N²-({2-[3-(trifluoromethoxy)phenyl][1,2,4]triazolo[1,5-c]quinazolin-5-yl}-D-serinamide;
(2R)-2-({2-[3-(trifluoromethoxy)phenyl][1,2,4]triazolo[1,5-c]quinazolin-5-yl}amino)butanamide;
(3S)-3-({2-[3-(trifluoromethoxy)phenyl][1,2,4]triazolo[1,5-c]quinazolin-5-yl}amino)azepan-2-one;
N-methyl-N²-{2-[3-(trifluoromethoxy)phenyl][1,2,4]triazolo[1,5-c]quinazolin-5-yl}-D-norvalinamide;
N-butyl-N²-{2-[3-(trifluoromethoxy)phenyl][1,2,4]triazolo[1,5-c]quinazolin-5-yl}glycinamide;
N-ethyl-2-({2-[3-(trifluoromethoxy)phenyl][1,2,4]triazolo[1,5-c]quinazolin-5-yl}amino)butanamide;
(2R)—N-ethyl-2-({2-[3-(trifluoromethoxy)phenyl][1,2,4]triazolo[1,5-c]quinazolin-5-yl}amino)butanamide;
(2S)—N-ethyl-2-({2-[3-(trifluoromethoxy)phenyl][1,2,4]triazolo[1,5-c]quinazolin-5-yl}amino)butanamide;
N-propyl-N²-{2-[3-(trifluoromethoxy)phenyl][1,2,4]triazolo[1,5-c]quinazolin-5-yl}-D-alaninamide;
N²-({2-[3-(trifluoromethoxy)phenyl][1,2,4]triazolo[1,5-c]quinazolin-5-yl}norleucinamide;
N²-({2-[3-(trifluoromethoxy)phenyl][1,2,4]triazolo[1,5-c]quinazolin-5-yl}-D-norleucinamide;
N²-({2-[3-(trifluoromethoxy)phenyl][1,2,4]triazolo[1,5-c]quinazolin-5-yl}-L-norleucinamide;
N-[2-(dimethylamino)ethyl]-N²-({2-[3-(trifluoromethoxy)phenyl][1,2,4]triazolo[1,5-c]quinazolin-5-yl}-D-alaninamide;
(3R)-3-({2-[4-(trifluoromethoxy)phenyl][1,2,4]triazolo[1,5-c]quinazolin-5-yl}amino)azepan-2-one;
(3R)-3-({2-[4-(trifluoromethoxy)phenyl][1,2,4]triazolo[1,5-c]quinazolin-5-yl}amino)pyrrolidin-2-one;
(3R)-1-methyl-3-({2-[4-(trifluoromethoxy)phenyl][1,2,4]triazolo[1,5-c]quinazolin-5-yl}amino)pyrrolidin-2-one;
N²-{2-[4-(trifluoromethoxy)phenyl][1,2,4]triazolo[1,5-c]quinazolin-5-yl}-D-serinamide;
(2R)-2-({2-[4-(trifluoromethoxy)phenyl][1,2,4]triazolo[1,5-c]quinazolin-5-yl}amino)butanamide;
(3S)-3-({2-[4-(trifluoromethoxy)phenyl][1,2,4]triazolo[1,5-c]quinazolin-5-yl}amino)azepan-2-one;
(3R)-3-({2-[4-(trifluoromethoxy)phenyl][1,2,4]triazolo[1,5-c]quinazolin-5-yl}amino)piperidin-2-one;
(3R)-3-{[2-(4-methoxythiophen-3-yl)[1,2,4]triazolo[1,5-c]quinazolin-5-yl]amino}azepan-2-one;
(3R)-3-{[2-(4-methoxythiophen-3-yl)[1,2,4]triazolo[1,5-c]quinazolin-5-yl]amino}pyrrolidin-2-one;
(3R)-3-{[2-(4-methoxythiophen-3-yl)[1,2,4]triazolo[1,5-c]quinazolin-5-yl]amino}piperidin-2-one;
(3S)-3-{[2-(4-methoxythiophen-3-yl)[1,2,4]triazolo[1,5-c]quinazolin-5-yl]amino}azepan-2-one;
(3R)-3-{[2-(4-methoxythiophen-3-yl)[1,2,4]triazolo[1,5-c]quinazolin-5-yl]amino}-1-methylpyrrolidin-2-one;
(2R)-2-{[2-(4-methoxythiophen-3-yl)[1,2,4]triazolo[1,5-c]quinazolin-5-yl]amino}butanamide;
N²-[2-(4-methoxythiophen-3-yl)[1,2,4]triazolo[1,5-c]quinazolin-5-yl]-N-methyl-D-norvalinamide;
(3R)-3-{[2-(thiophen-3-yl)[1,2,4]triazolo[1,5-c]quinazolin-5-yl]amino}azepan-2-one;
(3S)-3-{[2-(thiophen-3-yl)[1,2,4]triazolo[1,5-c]quinazolin-5-yl]amino}azepan-2-one;
(3R)-3-{[2-(thiophen-3-yl)[1,2,4]triazolo[1,5-c]quinazolin-5-yl]amino}pyrrolidin-2-one;
(3R)-3-{[2-(thiophen-3-yl)[1,2,4]triazolo[1,5-c]quinazolin-5-yl]amino}piperidin-2-one;
(2R)-2-{[2-(thiophen-3-yl)[1,2,4]triazolo[1,5-c]quinazolin-5-yl]amino}butanamide;
(3R)-1-methyl-3-{[2-(thiophen-3-yl)[1,2,4]triazolo[1,5-c]quinazolin-5-yl]amino}pyrrolidin-2-one;
(3R)-3-{[2-(2-methylthiophen-3-yl)[1,2,4]triazolo[1,5-c]quinazolin-5-yl]amino}azepan-2-one;
(3S)-3-{[2-(2-methylthiophen-3-yl)[1,2,4]triazolo[1,5-c]quinazolin-5-yl]amino}azepan-2-one;
(3R)-3-{[2-(2-methylthiophen-3-yl)[1,2,4]triazolo[1,5-c]quinazolin-5-yl]amino}piperidin-2-one;
(3R)-3-{[2-(2-methylthiophen-3-yl)[1,2,4]triazolo[1,5-c]quinazolin-5-yl]amino}pyrrolidin-2-one;
(3R)-1-methyl-3-{[2-(2-methylthiophen-3-yl)[1,2,4]triazolo[1,5-c]quinazolin-5-yl]amino}pyrrolidin-2-one;
(2R)-2-{[2-(2-methylthiophen-3-yl)[1,2,4]triazolo[1,5-c]quinazolin-5-yl]amino}butanamide;
(3R)-3-{[2-(5-methylthiophen-3-yl)[1,2,4]triazolo[1,5-c]quinazolin-5-yl]amino}azepan-2-one;
(3S)-3-{[2-(5-methylthiophen-3-yl)[1,2,4]triazolo[1,5-c]quinazolin-5-yl]amino}azepan-2-one;
(3R)-3-{[2-(5-methylthiophen-3-yl)[1,2,4]triazolo[1,5-c]quinazolin-5-yl]amino}piperidin-2-one;
(3R)-1-methyl-3-{[2-(5-methylthiophen-3-yl)[1,2,4]triazolo[1,5-c]quinazolin-5-yl]amino}pyrrolidin-2-one;
(3R)-3-{[2-(5-methylthiophen-3-yl)[1,2,4]triazolo[1,5-c]quinazolin-5-yl]amino}pyrrolidin-2-one;
N-methyl-N²-[2-(5-methylthiophen-3-yl)[1,2,4]triazolo[1,5-c]quinazolin-5-yl]-D-norvalinamide;
(2R)-2-{[2-(5-methylthiophen-3-yl)[1,2,4]triazolo[1,5-c]quinazolin-5-yl]amino}butanamide;
(3R)-3-({2-[3,5-bis(trifluoromethyl)phenyl][1,2,4]triazolo[1,5-c]quinazolin-5-yl}amino)azepan-2-one;
(3R)-3-({2-[3,5-bis(trifluoromethyl)phenyl][1,2,4]triazolo[1,5-c]quinazolin-5-yl}amino)pyrrolidin-2-one;
(2R)-2-({2-[3,5-bis(trifluoromethyl)phenyl][1,2,4]triazolo[1,5-c]quinazolin-5-yl}amino)butanamide;
(3R)-3-({2-[5-(trifluoromethyl)pyridin-3-yl][1,2,4]triazolo[1,5-c]quinazolin-5-yl}amino)azepan-2-one;
(3R)-3-({2-[4-(trifluoromethyl)pyridin-2-yl][1,2,4]triazolo[1,5-c]quinazolin-5-yl}amino)azepan-2-one;
(3S)-3-({2-[4-(trifluoromethyl)pyridin-2-yl][1,2,4]triazolo[1,5-c]quinazolin-5-yl}amino)azepan-2-one;
(3R)-3-({2-[4-(trifluoromethyl)pyridin-2-yl][1,2,4]triazolo[1,5-c]quinazolin-5-yl}amino)piperidin-2-one;
(3R)-3-({2-[4-(trifluoromethyl)pyridin-2-yl][1,2,4]triazolo[1,5-c]quinazolin-5-yl}amino)pyrrolidin-2-one;
(2R)-2-({2-[4-(trifluoromethyl)pyridin-2-yl][1,2,4]triazolo[1,5-c]quinazolin-5-yl}amino)butanamide;
N-methyl-N²-{2-[4-(trifluoromethyl)pyridin-2-yl][1,2,4]triazolo[1,5-c]quinazolin-5-yl}-D-norvalinamide;
(3R)-3-{[2-(3-fluoropyridin-4-yl)[1,2,4]triazolo[1,5-c]quinazolin-5-yl]amino}azepan-2-one;
(3R)-3-({2-[6-(trifluoromethyl)pyridin-2-yl][1,2,4]triazolo[1,5-c]quinazolin-5-yl}amino)azepan-2-one;
(2R)-2-({2-[6-(trifluoromethyl)pyridin-2-yl][1,2,4]triazolo[1,5-c]quinazolin-5-yl}amino)butanamide;
(3R)-3-({2-[4-(methanesulfonyl)phenyl][1,2,4]triazolo[1,5-c]quinazolin-5-yl}amino)azepan-2-one;
(3R)-3-({2-[4-(methanesulfonyl)phenyl][1,2,4]triazolo[1,5-c]quinazolin-5-yl}amino)pyrrolidin-2-one;
(3R)-3-({2-[4-(methanesulfonyl)phenyl][1,2,4]triazolo[1,5-c]quinazolin-5-yl}amino)piperidin-2-one;
(3S)-3-({2-[4-(methanesulfonyl)phenyl][1,2,4]triazolo[1,5-c]quinazolin-5-yl}amino)azepan-2-one;
(2R)-2-({2-[4-(methanesulfonyl)phenyl][1,2,4]triazolo[1,5-c]quinazolin-5-yl}amino)butanamide;
N²-{2-[4-(methanesulfonyl)phenyl][1,2,4]triazolo[1,5-c]quinazolin-5-yl}-N-methyl-D-norvalinamide;
(2R)-2-({2-[3-(methanesulfonyl)phenyl][1,2,4]triazolo[1,5-c]quinazolin-5-yl}amino)butanamide;
(2R)-2-{[2-(4-methoxyphenyl)[1,2,4]triazolo[1,5-c]quinazolin-5-yl]amino}butanamide;
3-(5-{[(3R)-2-oxoazepan-3-yl]amino}[1,2,4]triazolo[1,5-c]quinazolin-2-yl)benzonitrile;
(2R)-2-{[2-(3-cyanophenyl)[1,2,4]triazolo[1,5-c]quinazolin-5-yl]amino}butanamide;
3-(5-{[(3R)-2-oxopyrrolidin-3-yl]amino}[1,2,4]triazolo[1,5-c]quinazolin-2-yl)benzonitrile;
3-(5-{[(3R)-2-oxopiperidin-3-yl]amino}[1,2,4]triazolo[1,5-c]quinazolin-2-yl)benzonitrile;
3-(5-{[(3S)-2-oxoazepan-3-yl]amino}[1,2,4]triazolo[1,5-c]quinazolin-2-yl)benzonitrile;
(2R)-2-{[2-(4-cyanophenyl)[1,2,4]triazolo[1,5-c]quinazolin-5-yl]amino}butanamide;
4-(5-{[(3R)-2-oxoazepan-3-yl]amino}[1,2,4]triazolo[1,5-c]quinazolin-2-yl)benzonitrile;
4-(5-{[(3R)-2-oxopyrrolidin-3-yl]amino}[1,2,4]triazolo[1,5-c]quinazolin-2-yl)benzonitrile;
4-(5-{[(3R)-2-oxopiperidin-3-yl]amino}[1,2,4]triazolo[1,5-c]quinazolin-2-yl)benzonitrile;
(3R)-3-{[2-(imidazo[1,2-a]pyridin-7-yl)[1,2,4]triazolo[1,5-c]quinazolin-5-yl]amino}azepan-2-one;
N²-[2-(4-methoxyphenyl)[1,2,4]triazolo[1,5-c]quinazolin-5-yl]-N-propan-2-yl-D-alaninamide;
(3R)-3-{[2-(4-methoxyphenyl)-9-(propan-2-yl)[1,2,4]triazolo[1,5-c]quinazolin-5-yl]amino}azepan-2-one;
(3R)-3-{[2-(4-fluorophenyl)-7-(propan-2-yl)[1,2,4]triazolo[1,5-c]quinazolin-5-yl]amino}azepan-2-one;
(3R)-3-{[2-(3-fluorophenyl)-7-(propan-2-yl)[1,2,4]triazolo[1,5-c]quinazolin-5-yl]amino}azepan-2-one;
(3R)-3-{[2-(4-methoxyphenyl)-8-(propan-2-yl)[1,2,4]triazolo[1,5-c]quinazolin-5-yl]amino}azepan-2-one;
(3R)-3-{[2-(4-methoxyphenyl)-10-(oxetan-3-yl)[1,2,4]triazolo[1,5-c]quinazolin-5-yl]amino}azepan-2-one;
(3R)-3-{[2-(4-methoxyphenyl)-10-(oxan-4-yl)[1,2,4]triazolo[1,5-c]quinazolin-5-yl]amino}azepan-2-one;
(3R)-3-({2-(4-methoxyphenyl)-7-[1-(trifluoromethyl)cyclopropyl][1,2,4]triazolo[1,5-c]quinazolin-5-yl}amino)azepan-2-one;
(3R)-3-{[2-(4-methoxyphenyl)-7-(1-methylcyclopropyl)[1,2,4]triazolo[1,5-c]quinazolin-5-yl]amino}azepan-2-one;
2-(4-methoxyphenyl)-5-{1[(3R)-2-oxoazepan-3-yl]amino}[1,2,4]triazolo[1,5-c]quinazoline-8-carbonitrile;
2-(4-methoxyphenyl)-5-{1[(3R)-2-oxoazepan-3-yl]amino}[1,2,4]triazolo[1,5-c]quinazoline-9-carbonitrile;
2-(4-fluorophenyl)-5-{1[(3R)-2-oxoazepan-3-yl]amino}[1,2,4]triazolo[1,5-c]quinazoline-7-carbonitrile;
2-(1-methyl-1H-pyrazol-4-yl)-5-{1[(3R)-2-oxoazepan-3-yl]amino}[1,2,4]triazolo[1,5-c]quinazoline-7-carbonitrile;
(3R)-3-{[10-(difluoromethyl)-9-methyl-2-(1-methyl-1H-pyrazol-4-yl)[1,2,4]triazolo[1,5-c]quinazolin-5-yl]amino}azepan-2-one;
(3R)-3-{[8-(difluoromethyl)-9-methyl-2-(1-methyl-1H-pyrazol-4-yl)[1,2,4]triazolo[1,5-c]quinazolin-5-yl]amino}azepan-2-one;
(3R)-3-{[7-(difluoromethyl)-9-methyl-2-(1-methyl-1H-pyrazol-4-yl)[1,2,4]triazolo[1,5-c]quinazolin-5-yl]amino}azepan-2-one;
(3R)-3-({9-methyl-2-[1-methyl-3-(trifluoromethyl)-1H-pyrazol-4-yl][1,2,4]triazolo[1,5-c]quinazolin-5-yl}amino)azepan-2-one;
(6R)-6-({2-[3-(dimethylamino)phenyl][1,2,4]triazolo[1,5-c]quinazolin-5-yl}amino)-1,4-diazepan-5-one;
(6R)-6-{[2-(3,4-dimethoxyphenyl)[1,2,4]triazolo[1,5-c]quinazolin-5-yl]amino}-1,4-diazepan-5-one;
(6R)-6-{[2-(4-methoxy-2-methylphenyl)[1,2,4]triazolo[1,5-c]quinazolin-5-yl]amino}-1,4-diazepan-5-one;
(6R)-6-({2-[4-(cyclopropyloxy)phenyl][1,2,4]triazolo[1,5-c]quinazolin-5-yl}amino)-1,4-diazepan-5-one;
(6R)-6-{[2-(5-bromofuran-2-yl)[1,2,4]triazolo[1,5-c]quinazolin-5-yl]amino}-1,4-diazepan-5-one;
(6R)-6-{[2-(3-aminophenyl)[1,2,4]triazolo[1,5-c]quinazolin-5-yl]amino}-1,4-diazepan-5-one;
(6R)-6-({2-[1-(2-hydroxyethyl)-1H-pyrazol-4-yl][1,2,4]triazolo[1,5-c]quinazolin-5-yl}amino)-1,4-diazepan-5-one;
(6R)-6-[(2-{4-[(propan-2-yl)oxy]phenyl}[1,2,4]triazolo[1,5-c]quinazolin-5-yl)amino]-1,4-diazepan-5-one;
(6R)-6-({2-[1-(cyclopropylmethyl)-1H-pyrazol-4-yl][1,2,4]triazolo[1,5-c]quinazolin-5-yl}amino)-1,4-diazepan-5-one;
(6R)-6-{[10-chloro-2-(1-methyl-1H-pyrazol-4-yl)[1,2,4]triazolo[1,5-c]quinazolin-5-yl]amino}-1,4-diazepan-5-one;
(6R)-6-{[2-(4-methoxyphenyl)-9-(trifluoromethyl)[1,2,4]triazolo[1,5-c]quinazolin-5-yl]amino}-1,4-diazepan-5-one;
(6R)-6-{[7-chloro-2-(3-fluorophenyl)[1,2,4]triazolo[1,5-c]quinazolin-5-yl]amino}-1,4-diazepan-5-one;
(6R)-6-{[7-chloro-2-(4-fluorophenyl)[1,2,4]triazolo[1,5-c]quinazolin-5-yl]amino}-1,4-diazepan-5-one;
(6R)-6-{[7-chloro-2-(4-methoxyphenyl)[1,2,4]triazolo[1,5-c]quinazolin-5-yl]amino}-1,4-diazepan-5-one;
(6R)-6-{[2-(4-methoxyphenyl)-7-(trifluoromethyl)[1,2,4]triazolo[1,5-c]quinazolin-5-yl]amino}-1,4-diazepan-5-one;
(6R)-6-{[2-(4-fluorophenyl)-7-(trifluoromethyl)[1,2,4]triazolo[1,5-c]quinazolin-5-yl]amino}-1,4-diazepan-5-one;
(6R)-6-{[2-(3-fluorophenyl)-7-(trifluoromethyl)[1,2,4]triazolo[1,5-c]quinazolin-5-yl]amino}-1,4-diazepan-5-one;
(6R)-6-{[2-(1-methyl-1H-pyrazol-4-yl)-7-(trifluoromethyl)[1,2,4]triazolo[1,5-c]quinazolin-5-yl]amino}-1,4-diazepan-5-one;
(6R)-6-{[7-fluoro-2-(4-methoxyphenyl)[1,2,4]triazolo[1,5-c]quinazolin-5-yl]amino}-1,4-diazepan-5-one;
(6R)-6-{[7-fluoro-2-(4-fluorophenyl)[1,2,4]triazolo[1,5-c]quinazolin-5-yl]amino}-1,4-diazepan-5-one;
(6R)-6-{[7-fluoro-2-(1-methyl-1H-pyrazol-4-yl)[1,2,4]triazolo[1,5-c]quinazolin-5-yl]amino}-1,4-diazepan-5-one;
(6R)-6-{[2-(4-methoxyphenyl)-7-(propan-2-yl)[1,2,4]triazolo[1,5-c]quinazolin-5-yl]amino}-1,4-diazepan-5-one;
(6R)-6-({2-(1-methyl-1H-pyrazol-4-yl)-7-[1-(trifluoromethyl)cyclopropyl][1,2,4]triazolo[1,5-c]quinazolin-5-yl}amino)-1,4-diazepan-5-one;
(6R)-6-({2-(4-methoxyphenyl)-7-[1-(trifluoromethyl)cyclopropyl][1,2,4]triazolo[1,5-c]quinazolin-5-yl}amino)-1,4-diazepan-5-one;
(3R)-3-({2-[1-(2-methoxyethyl)-1H-pyrazol-4-yl][1,2,4]triazolo[1,5-c]quinazolin-5-yl}amino)azepan-2-one;
3-{[2-(4-methoxyphenyl)[1,2,4]triazolo[1,5-c]quinazolin-5-yl]amino}azocan-2-one;
(2S)-2-{[2-(4-methoxyphenyl)[1,2,4]triazolo[1,5-c]quinazolin-5-yl]amino}butanamide;
(3R)-3-{[2-(1-methyl-1H-1,2,3-triazol-4-yl)[1,2,4]triazolo[1,5-c]quinazolin-5-yl]amino}azepan-2-one;
1-{[2-(4-methoxyphenyl)[1,2,4]triazolo[1,5-c]quinazolin-5-yl]amino}cyclopropane-1-carboxamide;
4-(5-{1[(3S)-2-oxoazepan-3-yl]amino}[1,2,4]triazolo[1,5-c]quinazolin-2-yl)benzonitrile;
N-butyl-N²-{2-[4-(methanesulfonyl)phenyl][1,2,4]triazolo[1,5-c]quinazolin-5-yl}glycinamide;
N²-{2-[4-(methanesulfonyl)phenyl][1,2,4]triazolo[1,5-c]quinazolin-5-yl}-N-propyl-D-alaninamide;
(3R)-3-({2-[4-bromo-2-(trifluoromethoxy)phenyl][1,2,4]triazolo[1,5-c]quinazolin-5-yl}amino)azepan-2-one;
N-butyl-N²-[2-(3-cyanophenyl)[1,2,4]triazolo[1,5-c]quinazolin-5-yl]alaninamide;
(2S)-2-{[2-(3-cyanophenyl)[1,2,4]triazolo[1,5-c]quinazolin-5-yl]amino}butanamide;
3-(5-{[2-oxoazocan-3-yl]amino}[1,2,4]triazolo[1,5-c]quinazolin-2-yl)benzonitrile;
(3R)-3-({2-[3-(methanesulfonyl)phenyl][1,2,4]triazolo[1,5-c]quinazolin-5-yl}amino)azepan-2-one;
(2R)-2-{[2-(3-bromophenyl)[1,2,4]triazolo[1,5-c]quinazolin-5-yl]amino}butanamide;
(3R)-3-({2-[3-(dimethylamino)phenyl][1,2,4]triazolo[1,5-c]quinazolin-5-yl}amino)azepan-2-one;
3-({2-[3-(trifluoromethoxy)phenyl][1,2,4]triazolo[1,5-c]quinazolin-5-yl}amino)azocan-2-one;
(2S)-2-({2-[3-(trifluoromethoxy)phenyl][1,2,4]triazolo[1,5-c]quinazolin-5-yl}amino)butanamide;
(3R)-3-({2-[3-(methylsulfanyl)phenyl][1,2,4]triazolo[1,5-c]quinazolin-5-yl}amino)azepan-2-one;
(2S)-2-({2-[2-(methanesulfonyl)phenyl][1,2,4]triazolo[1,5-c]quinazolin-5-yl}amino)butanamide;
(2R)-2-({2-[2-(methanesulfonyl)phenyl][1,2,4]triazolo[1,5-c]quinazolin-5-yl}amino)butanamide;
(3R)-3-{[2-(2-bromophenyl)[1,2,4]triazolo[1,5-c]quinazolin-5-yl]amino}azepan-2-one;
(3S)-3-{[2-(2-bromophenyl)[1,2,4]triazolo[1,5-c]quinazolin-5-yl]amino}azepan-2-one;
(3R)-3-{[2-(2-bromophenyl)[1,2,4]triazolo[1,5-c]quinazolin-5-yl]amino}piperidin-2-one;
(3R)-3-({2-[2-(dimethylamino)phenyl][1,2,4]triazolo[1,5-c]quinazolin-5-yl}amino)azepan-2-one;
(3R)-3-({2-[2-(trifluoromethoxy)phenyl][1,2,4]triazolo[1,5-c]quinazolin-5-yl}amino)azepan-2-one;
(3S)-3-({2-[2-(trifluoromethoxy)phenyl][1,2,4]triazolo[1,5-c]quinazolin-5-yl}amino)azepan-2-one;
(3R)-3-({2-[2-(trifluoromethoxy)phenyl][1,2,4]triazolo[1,5-c]quinazolin-5-yl}amino)piperidin-2-one;
(3R)-3-({2-[2-(trifluoromethoxy)phenyl][1,2,4]triazolo[1,5-c]quinazolin-5-yl}amino)pyrrolidin-2-one;
(3R)-1-methyl-3-({2-[2-(trifluoromethoxy)phenyl][1,2,4]triazolo[1,5-c]quinazolin-5-yl}amino)pyrrolidin-2-one;
3-({2-[2-(trifluoromethoxy)phenyl][1,2,4]triazolo[1,5-c]quinazolin-5-yl}amino)azocan-2-one;
(2S)-2-({2-[2-(trifluoromethoxy)phenyl][1,2,4]triazolo[1,5-c]quinazolin-5-yl}amino)butanamide;
N-ethyl-2-({2-[2-(trifluoromethoxy)phenyl][1,2,4]triazolo[1,5-c]quinazolin-5-yl}amino)butanamide;
N-methyl-N²-{2-[2-(trifluoromethoxy)phenyl][1,2,4]triazolo[1,5-c]quinazolin-5-yl}-D-norvalinamide;
N-propyl-N²-{2-[2-(trifluoromethoxy)phenyl][1,2,4]triazolo[1,5-c]quinazolin-5-yl}-D-alaninamide;
N-butyl-N²-{2-[2-(trifluoromethoxy)phenyl][1,2,4]triazolo[1,5-c]quinazolin-5-yl}glycinamide;
N²-{2-[2-(trifluoromethoxy)phenyl][1,2,4]triazolo[1,5-c]quinazolin-5-yl}norleucinamide;
N²-{2-[2-(trifluoromethoxy)phenyl][1,2,4]triazolo[1,5-c]quinazolin-5-yl}-D-serinamide;
(2R)-2-({2-[2-(trifluoromethoxy)phenyl][1,2,4]triazolo[1,5-c]quinazolin-5-yl}amino)butanamide;
(3R)-3-({2-[2-(difluoromethoxy)phenyl][1,2,4]triazolo[1,5-c]quinazolin-5-yl}amino)azepan-2-one;
(3S)-3-({2-[2-(difluoromethoxy)phenyl][1,2,4]triazolo[1,5-c]quinazolin-5-yl}amino)azepan-2-one;
(3R)-3-({2-[2-(difluoromethoxy)phenyl][1,2,4]triazolo[1,5-c]quinazolin-5-yl}amino)piperidin-2-one;
(3R)-3-({2-[2-(difluoromethoxy)phenyl][1,2,4]triazolo[1,5-c]quinazolin-5-yl}amino)pyrrolidin-2-one;
3-({2-[2-(difluoromethoxy)phenyl][1,2,4]triazolo[1,5-c]quinazolin-5-yl}amino)azocan-2-one;
(2S)-2-({2-[2-(difluoromethoxy)phenyl][1,2,4]triazolo[1,5-c]quinazolin-5-yl}amino)butanamide;
(2R)-2-({2-[2-(difluoromethoxy)phenyl][1,2,4]triazolo[1,5-c]quinazolin-5-yl}amino)butanamide;
N²-{2-[2-(difluoromethoxy)phenyl][1,2,4]triazolo[1,5-c]quinazolin-5-yl}-N-methyl-D-norvalinamide;
(3R)-3-({2-[2-(methylsulfanyl)phenyl][1,2,4]triazolo[1,5-c]quinazolin-5-yl}amino)azepan-2-one;
(3R)-3-{[2-(5-fluoropyridin-3-yl)[1,2,4]triazolo[1,5-c]quinazolin-5-yl]amino}azepan-2-one;
(3R)-3-{[2-(5-chloropyridin-3-yl)[1,2,4]triazolo[1,5-c]quinazolin-5-yl]amino}azepan-2-one;
(3S)-3-{[2-(3-fluoropyridin-4-yl)[1,2,4]triazolo[1,5-c]quinazolin-5-yl]amino}azepan-2-one;
(3R)-3-{[2-(3-fluoropyridin-4-yl)[1,2,4]triazolo[1,5-c]quinazolin-5-yl]amino}piperidin-2-one;
(3R)-3-{[2-(3-fluoropyridin-4-yl)[1,2,4]triazolo[1,5-c]quinazolin-5-yl]amino}pyrrolidin-2-one;
(2R)-2-{[2-(3-fluoropyridin-4-yl)[1,2,4]triazolo[1,5-c]quinazolin-5-yl]amino}butanamide;
N-butyl-N²-[2-(3-fluoropyridin-4-yl)[1,2,4]triazolo[1,5-c]quinazolin-5-yl]glycinamide;
N²-[2-(3-fluoropyridin-4-yl)[1,2,4]triazolo[1,5-c]quinazolin-5-yl]-N-methyl-D-norvalinamide;
N²-[2-(3-fluoropyridin-4-yl)[1,2,4]triazolo[1,5-c]quinazolin-5-yl]-N-propyl-D-alaninamide;
(3R)-3-({2-[2-(trifluoromethyl)pyridin-3-yl][1,2,4]triazolo[1,5-c]quinazolin-5-yl}amino)azepan-2-one;
(2R)-2-({2-[2-(trifluoromethyl)pyridin-3-yl][1,2,4]triazolo[1,5-c]quinazolin-5-yl}amino)butanamide;
(3R)-3-({2-[4-(trifluoromethyl)pyridin-3-yl][1,2,4]triazolo[1,5-c]quinazolin-5-yl}amino)azepan-2-one;
(2R)-2-({2-[4-(trifluoromethyl)pyridin-3-yl][1,2,4]triazolo[1,5-c]quinazolin-5-yl}amino)butanamide;
(3R)-3-({2-[5-fluoro-2-(trifluoromethoxy)phenyl][1,2,4]triazolo[1,5-c]quinazolin-5-yl}amino)azepan-2-one;
(3S)-3-({2-[5-fluoro-2-(trifluoromethoxy)phenyl][1,2,4]triazolo[1,5-c]quinazolin-5-yl}amino)azepan-2-one;
N²-{2-[5-fluoro-2-(trifluoromethoxy)phenyl][1,2,4]triazolo[1,5-c]quinazolin-5-yl}-N-methyl-D-norvalinamide;
N²-{2-[5-fluoro-2-(trifluoromethoxy)phenyl][1,2,4]triazolo[1,5-c]quinazolin-5-yl}-N-propyl-D-alaninamide;
(2R)-2-({2-[5-fluoro-2-(trifluoromethoxy)phenyl][1,2,4]triazolo[1,5-c]quinazolin-5-yl}amino)butanamide;
(3R)-3-({2-[5-chloro-2-(trifluoromethoxy)phenyl][1,2,4]triazolo[1,5-c]quinazolin-5-yl}amino)azepan-2-one;
(3S)-3-({2-[5-chloro-2-(trifluoromethoxy)phenyl][1,2,4]triazolo[1,5-c]quinazolin-5-yl}amino)azepan-2-one;
(3R)-3-({2-[5-chloro-2-(trifluoromethoxy)phenyl][1,2,4]triazolo[1,5-c]quinazolin-5-yl}amino)piperidin-2-one;
(3R)-3-({2-[5-chloro-2-(trifluoromethoxy)phenyl][1,2,4]triazolo[1,5-c]quinazolin-5-yl}amino)pyrrolidin-2-one;
N²-{2-[5-chloro-2-(trifluoromethoxy)phenyl][1,2,4]triazolo[1,5-c]quinazolin-5-yl}-N-methyl-D-norvalinamide;
N²-{2-[5-chloro-2-(trifluoromethoxy)phenyl][1,2,4]triazolo[1,5-c]quinazolin-5-yl}-N-propyl-D-alaninamide;
N-butyl-N²-{2-[5-chloro-2-(trifluoromethoxy)phenyl][1,2,4]triazolo[1,5-c]quinazolin-5-yl}glycinamide;
(2R)-2-({2-[5-chloro-2-(trifluoromethoxy)phenyl][1,2,4]triazolo[1,5-c]quinazolin-5-yl}amino)butanamide;
(3R)-3-({2-[5-bromo-2-(trifluoromethoxy)phenyl][1,2,4]triazolo[1,5-c]quinazolin-5-yl}amino)azepan-2-one;
(3S)-3-({2-[5-bromo-2-(trifluoromethoxy)phenyl][1,2,4]triazolo[1,5-c]quinazolin-5-yl}amino)azepan-2-one;
(2R)-2-({2-[5-bromo-2-(trifluoromethoxy)phenyl][1,2,4]triazolo[1,5-c]quinazolin-5-yl}amino)butanamide;
(3R)-3-({2-[4-methoxy-2-(trifluoromethoxy)phenyl][1,2,4]triazolo[1,5-c]quinazolin-5-yl}amino)azepan-2-one;
(3S)-3-({2-[4-methoxy-2-(trifluoromethoxy)phenyl][1,2,4]triazolo[1,5-c]quinazolin-5-yl}amino)azepan-2-one;
N²-{2-[4-methoxy-2-(trifluoromethoxy)phenyl][1,2,4]triazolo[1,5-c]quinazolin-5-yl}-N-methyl-D-norvalinamide;
(2R)-2-({2-[4-methoxy-2-(trifluoromethoxy)phenyl][1,2,4]triazolo[1,5-c]quinazolin-5-yl}amino)butanamide;
(3R)-3-({2-[4-methoxy-2-(trifluoromethyl)phenyl][1,2,4]triazolo[1,5-c]quinazolin-5-yl}amino)azepan-2-one;
(3S)-3-({2-[4-methoxy-2-(trifluoromethyl)phenyl][1,2,4]triazolo[1,5-c]quinazolin-5-yl}amino)azepan-2-one;
(2S)-2-({2-[4-methoxy-2-(trifluoromethyl)phenyl][1,2,4]triazolo[1,5-c]quinazolin-5-yl}amino)butanamide;
(2R)-2-({2-[4-methoxy-2-(trifluoromethyl)phenyl][1,2,4]triazolo[1,5-c]quinazolin-5-yl}amino)butanamide;
(3R)-3-({2-[4-fluoro-2-(trifluoromethoxy)phenyl][1,2,4]triazolo[1,5-c]quinazolin-5-yl}amino)azepan-2-one;
(3R)-3-({2-[4-chloro-2-(trifluoromethoxy)phenyl][1,2,4]triazolo[1,5-c]quinazolin-5-yl}amino)azepan-2-one;
(3S)-3-({2-[4-chloro-2-(trifluoromethoxy)phenyl][1,2,4]triazolo[1,5-c]quinazolin-5-yl}amino)azepan-2-one;
(3R)-3-({2-[4-chloro-2-(trifluoromethoxy)phenyl][1,2,4]triazolo[1,5-c]quinazolin-5-yl}amino)piperidin-2-one;
(3R)-3-({2-[4-chloro-2-(trifluoromethoxy)phenyl][1,2,4]triazolo[1,5-c]quinazolin-5-yl}amino)pyrrolidin-2-one;
N-butyl-N²-{2-[4-chloro-2-(trifluoromethoxy)phenyl][1,2,4]triazolo[1,5-c]quinazolin-5-yl}alaninamide;
(2S)-2-({2-[4-chloro-2-(trifluoromethoxy)phenyl][1,2,4]triazolo[1,5-c]quinazolin-5-yl}amino)butanamide;
(2R)-2-({2-[4-chloro-2-(trifluoromethoxy)phenyl][1,2,4]triazolo[1,5-c]quinazolin-5-yl}amino)butanamide;
(2S)-2-({2-[4-bromo-2-(trifluoromethoxy)phenyl][1,2,4]triazolo[1,5-c]quinazolin-5-yl}amino)butanamide;
(3R)-3-({2-[4-chloro-2-(difluoromethoxy)phenyl][1,2,4]triazolo[1,5-c]quinazolin-5-yl}amino)azepan-2-one;
(3S)-3-({2-[4-chloro-2-(difluoromethoxy)phenyl][1,2,4]triazolo[1,5-c]quinazolin-5-yl}amino)azepan-2-one;
(3R)-3-({2-[4-chloro-2-(difluoromethoxy)phenyl][1,2,4]triazolo[1,5-c]quinazolin-5-yl}amino)piperidin-2-one;
(3R)-3-({2-[4-chloro-2-(difluoromethoxy)phenyl][1,2,4]triazolo[1,5-c]quinazolin-5-yl}amino)pyrrolidin-2-one;
3-({2-[4-chloro-2-(difluoromethoxy)phenyl][1,2,4]triazolo[1,5-c]quinazolin-5-yl}amino)azocan-2-one;
(3S)-3-({2-[4-chloro-2-(difluoromethoxy)phenyl][1,2,4]triazolo[1,5-c]quinazolin-5-yl}amino)-1-methylazepan-2-one;
(2R)-2-({2-[4-chloro-2-(difluoromethoxy)phenyl][1,2,4]triazolo[1,5-c]quinazolin-5-yl}amino)butanamide;
(2S)-2-({2-[4-chloro-2-(difluoromethoxy)phenyl][1,2,4]triazolo[1,5-c]quinazolin-5-yl}amino)butanamide;
N-butyl-N²-{2-[4-chloro-2-(difluoromethoxy)phenyl][1,2,4]triazolo[1,5-c]quinazolin-5-yl}alaninamide;
(3R)-3-({2-[1-(difluoromethyl)-1H-pyrazol-4-yl][1,2,4]triazolo[1,5-c]quinazolin-5-yl}amino)azepan-2-one;
(3R)-3-{[2-(1-cyclopropyl-1H-pyrazol-4-yl)[1,2,4]triazolo[1,5-c]quinazolin-5-yl]amino}azepan-2-one;
(3R)-3-{[2-(3,5-dimethyl-1H-pyrazol-4-yl)[1,2,4]triazolo[1,5-c]quinazolin-5-yl]amino}azepan-2-one;
(3R)-3-{[2-(5-bromofuran-2-yl)[1,2,4]triazolo[1,5-c]quinazolin-5-yl]amino}azepan-2-one;
(3R)-3-{[2-(4-methylthiophen-3-yl)[1,2,4]triazolo[1,5-c]quinazolin-5-yl]amino}azepan-2-one;
(3S)-3-{[2-(4-methylthiophen-3-yl)[1,2,4]triazolo[1,5-c]quinazolin-5-yl]amino}azepan-2-one;
(3R)-3-{[2-(4-methylthiophen-3-yl)[1,2,4]triazolo[1,5-c]quinazolin-5-yl]amino}piperidin-2-one;
(3R)-3-{[2-(4-methylthiophen-3-yl)[1,2,4]triazolo[1,5-c]quinazolin-5-yl]amino}pyrrolidin-2-one;
(2R)-2-{[2-(4-methylthiophen-3-yl)[1,2,4]triazolo[1,5-c]quinazolin-5-yl]amino}butanamide;
(3R)-3-{[2-(4-fluorophenyl)-9-(trifluoromethyl)[1,2,4]triazolo[1,5-c]quinazolin-5-yl]amino}azepan-2-one;
(3R)-3-{[2-(2-fluorophenyl)-9-(trifluoromethyl)[1,2,4]triazolo[1,5-c]quinazolin-5-yl]amino}azepan-2-one;
(3R)-3-{[2-(4-methoxyphenyl)-9-(trifluoromethyl)[1,2,4]triazolo[1,5-c]quinazolin-5-yl]amino}azepan-2-one;
(3R)-3-{[2-(1-methyl-1H-pyrazol-4-yl)-9-(trifluoromethyl)[1,2,4]triazolo[1,5-c]quinazolin-5-yl]amino}azepan-2-one;
(3R)-3-{[9-cyclopropyl-2-(4-methoxyphenyl)[1,2,4]triazolo[1,5-c]quinazolin-5-yl]amino}azepan-2-one;
(3R)-3-{[2-(4-methoxyphenyl)-8-(trifluoromethyl)[1,2,4]triazolo[1,5-c]quinazolin-5-yl]amino}azepan-2-one;
(3R)-3-{[8-methoxy-2-(4-methoxyphenyl)[1,2,4]triazolo[1,5-c]quinazolin-5-yl]amino}azepan-2-one;
(3R)-3-{[8-methoxy-2-(1-methyl-1H-pyrazol-4-yl)[1,2,4]triazolo[1,5-c]quinazolin-5-yl]amino}azepan-2-one;
(3R)-3-{[7-fluoro-2-(1-methyl-1H-pyrazol-4-yl)[1,2,4]triazolo[1,5-c]quinazolin-5-yl]amino}azepan-2-one;
(3R)-3-{[7-fluoro-2-(pyridin-4-yl)[1,2,4]triazolo[1,5-c]quinazolin-5-yl]amino}azepan-2-one;
(3R)-3-{[7-chloro-2-(4-methoxyphenyl)[1,2,4]triazolo[1,5-c]quinazolin-5-yl]amino}azepan-2-one;
(3R)-3-{[7-chloro-2-(4-fluorophenyl)[1,2,4]triazolo[1,5-c]quinazolin-5-yl]amino}azepan-2-one;
(3R)-3-{[7-chloro-2-(3-fluorophenyl)[1,2,4]triazolo[1,5-c]quinazolin-5-yl]amino}azepan-2-one;
N²-[7-bromo-2-(4-methoxyphenyl)[1,2,4]triazolo[1,5-c]quinazolin-5-yl]-D-valinamide;
(2R)-2-{[7-bromo-2-(4-methoxyphenyl)[1,2,4]triazolo[1,5-c]quinazolin-5-yl]amino}butanamide;
4-(7-bromo-5-{[(3R)-2-oxoazepan-3-yl]amino}[1,2,4]triazolo[1,5-c]quinazolin-2-yl)benzonitrile;
4-(7-bromo-5-{[(3S)-2-oxoazepan-3-yl]amino}[1,2,4]triazolo[1,5-c]quinazolin-2-yl)benzonitrile;
4-(7-bromo-5-1{[(3R)-2-oxopiperidin-3-yl]amino}[1,2,4]triazolo[1,5-c]quinazolin-2-yl)benzonitrile;
4-(7-bromo-5-1{[(3R)-2-oxopyrrolidin-3-yl]amino}[1,2,4]triazolo[1,5-c]quinazolin-2-yl)benzonitrile;
N²-[7-bromo-2-(4-cyanophenyl)[1,2,4]triazolo[1,5-c]quinazolin-5-yl]-N-methyl-D-norvalinamide;
(2R)-2-{[7-bromo-2-(4-cyanophenyl)[1,2,4]triazolo[1,5-c]quinazolin-5-yl]amino}butanamide;
(2S)-2-{[7-bromo-2-(4-cyanophenyl)[1,2,4]triazolo[1,5-c]quinazolin-5-yl]amino}butanamide;
(3R)-3-({7-bromo-2-[4-methoxy-2-(trifluoromethoxy)phenyl][1,2,4]triazolo[1,5-c]quinazolin-5-yl}amino)azepan-2-one;
(3S)-3-({7-bromo-2-[4-methoxy-2-(trifluoromethoxy)phenyl][1,2,4]triazolo[1,5-c]quinazolin-5-yl}amino)azepan-2-one;
(2R)-2-({7-bromo-2-[4-methoxy-2-(trifluoromethoxy)phenyl][1,2,4]triazolo[1,5-c]quinazolin-5-yl}amino)butanamide;
(2S)-2-({7-bromo-2-[4-methoxy-2-(trifluoromethoxy)phenyl][1,2,4]triazolo[1,5-c]quinazolin-5-yl}amino)butanamide;
(3R)-3-({7-bromo-2-[4-methoxy-2-(trifluoromethyl)phenyl][1,2,4]triazolo[1,5-c]quinazolin-5-yl}amino)azepan-2-one;
(3S)-3-({7-bromo-2-[4-methoxy-2-(trifluoromethyl)phenyl][1,2,4]triazolo[1,5-c]quinazolin-5-yl}amino)azepan-2-one;
(3R)-3-({7-bromo-2-[4-chloro-2-(trifluoromethoxy)phenyl][1,2,4]triazolo[1,5-c]quinazolin-5-yl}amino)azepan-2-one;
(3S)-3-({7-bromo-2-[4-chloro-2-(trifluoromethoxy)phenyl][1,2,4]triazolo[1,5-c]quinazolin-5-yl}amino)azepan-2-one;
(2R)-2-({7-bromo-2-[4-chloro-2-(trifluoromethoxy)phenyl][1,2,4]triazolo[1,5-c]quinazolin-5-yl}amino)butanamide;
(2S)-2-({7-bromo-2-[4-chloro-2-(trifluoromethoxy)phenyl][1,2,4]triazolo[1,5-c]quinazolin-5-yl}amino)butanamide;
(3R)-3-({7-bromo-2-[4-chloro-2-(difluoromethoxy)phenyl][1,2,4]triazolo[1,5-c]quinazolin-5-yl}amino)azepan-2-one;
(3S)-3-({7-bromo-2-[4-chloro-2-(difluoromethoxy)phenyl][1,2,4]triazolo[1,5-c]quinazolin-5-yl}amino)azepan-2-one;
(2R)-2-({7-bromo-2-[4-chloro-2-(difluoromethoxy)phenyl][1,2,4]triazolo[1,5-c]quinazolin-5-yl}amino)butanamide;
N²-[7-bromo-2-(1-methyl-1H-pyrazol-4-yl)[1,2,4]triazolo[1,5-c]quinazolin-5-yl]-D-valinamide;
(3R)-3-{[7-bromo-2-(1H-pyrazol-4-yl)[1,2,4]triazolo[1,5-c]quinazolin-5-yl]amino}azepan-2-one;
(3S)-3-{[7-bromo-2-(1-methyl-1H-pyrazol-4-yl)[1,2,4]triazolo[1,5-c]quinazolin-5-yl]amino}azepan-2-one;
(3R)-3-{[2-(3-fluorophenyl)-7-(trifluoromethyl)[1,2,4]triazolo[1,5-c]quinazolin-5-yl]amino}azepan-2-one;
(3S)-3-{[2-(4-methoxyphenyl)-7-(trifluoromethyl)[1,2,4]triazolo[1,5-c]quinazolin-5-yl]amino}azepan-2-one;
(3R)-3-{[2-(1-methyl-1H-pyrazol-4-yl)-7-(trifluoromethyl)[1,2,4]triazolo[1,5-c]quinazolin-5-yl]amino}azepan-2-one;
(3S)-3-{[2-(1-methyl-1H-pyrazol-4-yl)-7-(trifluoromethyl)[1,2,4]triazolo[1,5-c]quinazolin-5-yl]amino}azepan-2-one;
(3R)-3-{[2-(1-methyl-1H-pyrazol-4-yl)-7-(trifluoromethyl)[1,2,4]triazolo[1,5-c]quinazolin-5-yl]amino}piperidin-2-one;
(3R)-3-{[2-(1-methyl-1H-pyrazol-4-yl)-7-(trifluoromethyl)[1,2,4]triazolo[1,5-c]quinazolin-5-yl]amino}pyrrolidin-2-one;
(3R)-3-{[2-(4-methoxyphenyl)-7-(propan-2-yl)[1,2,4]triazolo[1,5-c]quinazolin-5-yl]amino}azepan-2-one;
(3R)-3-{[2-(1-methyl-1H-pyrazol-4-yl)-7-(propan-2-yl)[1,2,4]triazolo[1,5-c]quinazolin-5-yl]amino}azepan-2-one;
(3R)-3-{[8,9-dimethoxy-2-(1-methyl-1H-pyrazol-4-yl)[1,2,4]triazolo[1,5-c]quinazolin-5-yl]amino}azepan-2-one;
(3R)-3-{[8,9-dimethoxy-2-(4-methoxyphenyl)[1,2,4]triazolo[1,5-c]quinazolin-5-yl]amino}azepan-2-one;
(3R)-3-{[7,9-dibromo-2-(4-fluorophenyl)[1,2,4]triazolo[1,5-c]quinazolin-5-yl]amino}azepan-2-one;
(3R)-3-{[7,9-dibromo-2-(3-fluorophenyl)[1,2,4]triazolo[1,5-c]quinazolin-5-yl]amino}azepan-2-one;
(3R)-3-{[7,9-dibromo-2-(4-methoxyphenyl)[1,2,4]triazolo[1,5-c]quinazolin-5-yl]amino}azepan-2-one;
(3R)-3-{[7,9-dibromo-2-(1-methyl-1H-pyrazol-4-yl)[1,2,4]triazolo[1,5-c]quinazolin-5-yl]amino}azepan-2-one;
(3R)-3-({2-[1-(oxetan-3-yl)-1H-pyrazol-4-yl][1,2,4]triazolo[1,5-c]quinazolin-5-yl}amino)azepan-2-one;
(3R)-3-({2-[1-(1-methylpiperidin-4-yl)-1H-pyrazol-4-yl][1,2,4]triazolo[1,5-c]quinazolin-5-yl}amino)azepan-2-one;
(3R)-3-({2-[1-(cyclobutylmethyl)-1H-pyrazol-4-yl][1,2,4]triazolo[1,5-c]quinazolin-5-yl}amino)azepan-2-one;
(3R)-3-({2-[1-(3-hydroxypropyl)-1H-pyrazol-4-yl][1,2,4]triazolo[1,5-c]quinazolin-5-yl}amino)azepan-2-one;
(3R)-3-[(2-{1-[2-(dimethylamino)ethyl]-1H-pyrazol-4-yl}[1,2,4]triazolo[1,5-c]quinazolin-5-yl)amino]azepan-2-one;
(3R)-3-({2-[1-(oxan-4-yl)-1H-pyrazol-4-yl][1,2,4]triazolo[1,5-c]quinazolin-5-yl}amino)azepan-2-one;
(3R)-3-({2-[1-(2-hydroxyethyl)-1H-pyrazol-4-yl][1,2,4]triazolo[1,5-c]quinazolin-5-yl}amino)azepan-2-one;
(3R)-3-({2-[1-(cyclopropylmethyl)-1H-pyrazol-4-yl][1,2,4]triazolo[1,5-c]quinazolin-5-yl}amino)azepan-2-one;
(3R)-3-{[2-(1-cyclobutyl-1H-pyrazol-4-yl)[1,2,4]triazolo[1,5-c]quinazolin-5-yl]amino}azepan-2-one;
(3R)-3-({2-[3-(trifluoromethyl)-1H-pyrazol-4-yl][1,2,4]triazolo[1,5-c]quinazolin-5-yl}amino)azepan-2-one;
(3R)-3-{[2-(1,3-dimethyl-1H-pyrazol-4-yl)[1,2,4]triazolo[1,5-c]quinazolin-5-yl]amino}azepan-2-one;
(3R)-3-{[2-(1,2,5-trimethyl-1H-pyrrol-3-yl)[1,2,4]triazolo[1,5-c]quinazolin-5-yl]amino}azepan-2-one;
1,5-dimethyl-3-(5-{[(3R)-2-oxoazepan-3-yl]amino}[1,2,4]triazolo[1,5-c]quinazolin-2-yl)-1H-pyrrole-2-carbonitrile;
(3R)-3-{[2-(1-tert-butyl-1H-pyrazol-5-yl)[1,2,4]triazolo[1,5-c]quinazolin-5-yl]amino}azepan-2-one;
3R)-3-{[2-(5-methyl-1H-pyrazol-4-yl)[1,2,4]triazolo[1,5-c]quinazolin-5-yl]amino}azepan-2-one;
(3R)-3-{[2-(1-methyl-1H-imidazol-5-yl)[1,2,4]triazolo[1,5-c]quinazolin-5-yl]amino}azepan-2-one;
(3R)-3-{[2-(2-chlorothiophen-3-yl)[1,2,4]triazolo[1,5-c]quinazolin-5-yl]amino}azepan-2-one;
(3R)-3-({2-[4-(cyclopropyloxy)phenyl][1,2,4]triazolo[1,5-c]quinazolin-5-yl}amino)azepan-2-one;
(3R)-3-[(2-{4-[(propan-2-yl)oxy]phenyl}[1,2,4]triazolo[1,5-c]quinazolin-5-yl)amino]azepan-2-one;
(3R)-3-{[2-(4-hydroxyphenyl)[1,2,4]triazolo[1,5-c]quinazolin-5-yl]amino}azepan-2-one;
(3R)-3-({2-[4-(difluoromethyl)phenyl][1,2,4]triazolo[1,5-c]quinazolin-5-yl}amino)azepan-2-one;
(3R)-3-{[2-(4-ethoxyphenyl)[1,2,4]triazolo[1,5-c]quinazolin-5-yl]amino}azepan-2-one;
(3R)-3-({2-[4-(difluoromethoxy)phenyl][1,2,4]triazolo[1,5-c]quinazolin-5-yl}amino)azepan-2-one;
(3R)-3-{[2-(2-hydroxyphenyl)[1,2,4]triazolo[1,5-c]quinazolin-5-yl]amino}azepan-2-one;
2-(5-{1[(3R)-2-oxoazepan-3-yl]amino}[1,2,4]triazolo[1,5-c]quinazolin-2-yl)benzonitrile;
(3R)-3-{[2-(4-aminophenyl)[1,2,4]triazolo[1,5-c]quinazolin-5-yl]amino}azepan-2-one;
(3R)-3-{[2-(3-aminophenyl)[1,2,4]triazolo[1,5-c]quinazolin-5-yl]amino}azepan-2-one;
(3R)-3-{[2-(2-aminophenyl)[1,2,4]triazolo[1,5-c]quinazolin-5-yl]amino}azepan-2-one;
(3R)-3-{[2-(2,6-dimethylphenyl)[1,2,4]triazolo[1,5-c]quinazolin-5-yl]amino}azepan-2-one;
(3R)-3-{[2-(3,4-dimethoxyphenyl)[1,2,4]triazolo[1,5-c]quinazolin-5-yl]amino}azepan-2-one;
(3R)-3-{[2-(3-chloro-4-fluorophenyl)[1,2,4]triazolo[1,5-c]quinazolin-5-yl]amino}azepan-2-one;
(3R)-3-{[2-(4-fluoro-3-methylphenyl)[1,2,4]triazolo[1,5-c]quinazolin-5-yl]amino}azepan-2-one;
(3R)-3-{[2-(4-methoxy-3-methylphenyl)[1,2,4]triazolo[1,5-c]quinazolin-5-yl]amino}azepan-2-one;
(3R)-3-{[2-(4-methoxy-2-methylphenyl)[1,2,4]triazolo[1,5-c]quinazolin-5-yl]amino}azepan-2-one;
(3R)-3-{[2-(3-fluoro-4-hydroxyphenyl)[1,2,4]triazolo[1,5-c]quinazolin-5-yl]amino}azepan-2-one;
(3R)-3-({2-[2-fluoro-4-(methylsulfanyl)phenyl][1,2,4]triazolo[1,5-c]quinazolin-5-yl}amino)azepan-2-one;
(3R)-3-{[2-(2-fluoro-4-methoxyphenyl)[1,2,4]triazolo[1,5-c]quinazolin-5-yl]amino}azepan-2-one;
(3R)-3-[(2-{3-fluoro-4-[(propan-2-yl)oxy]phenyl}[1,2,4]triazolo[1,5-c]quinazolin-5-yl)amino]azepan-2-one;
(3R)-3-{[2-(3,4-difluorophenyl)[1,2,4]triazolo[1,5-c]quinazolin-5-yl]amino}azepan-2-one;
(3R)-3-{[2-(2-fluoro-4-hydroxyphenyl)[1,2,4]triazolo[1,5-c]quinazolin-5-yl]amino}azepan-2-one;
(3R)-3-{[2-(3-fluoro-4-methoxyphenyl)[1,2,4]triazolo[1,5-c]quinazolin-5-yl]amino}azepan-2-one;
(3R)-3-{[2-(5-methylpyridin-3-yl)[1,2,4]triazolo[1,5-c]quinazolin-5-yl]amino}azepan-2-one;
(3R)-3-{[2-(6-aminopyridin-3-yl)[1,2,4]triazolo[1,5-c]quinazolin-5-yl]amino}azepan-2-one;
(3R)-3-{[2-(2-fluoropyridin-4-yl)[1,2,4]triazolo[1,5-c]quinazolin-5-yl]amino}azepan-2-one;
(3R)-3-{[2-(6-fluoropyridin-3-yl)[1,2,4]triazolo[1,5-c]quinazolin-5-yl]amino}azepan-2-one;
(3R)-3-{[2-(2-fluoropyridin-3-yl)[1,2,4]triazolo[1,5-c]quinazolin-5-yl]amino}azepan-2-one;
(3R)-3-{[2-(5-fluoro-6-methylpyridin-3-yl)[1,2,4]triazolo[1,5-c]quinazolin-5-yl]amino}azepan-2-one;
(3R)-3-{[2-(pyrimidin-5-yl)[1,2,4]triazolo[1,5-c]quinazolin-5-yl]amino}azepan-2-one;
(3R)-3-({2-[2-(trifluoromethyl)pyrimidin-5-yl][1,2,4]triazolo[1,5-c]quinazolin-5-yl}amino)azepan-2-one;
(3R)-3-{[2-(pyridazin-3-yl)[1,2,4]triazolo[1,5-c]quinazolin-5-yl]amino}azepan-2-one;
(3R)-3-{[2-(1,2-thiazol-4-yl)[1,2,4]triazolo[1,5-c]quinazolin-5-yl]amino}azepan-2-one;
(3R)-3-({2-[1-(piperidin-4-yl)-1H-pyrazol-4-yl][1,2,4]triazolo[1,5-c]quinazolin-5-yl}amino)azepan-2-one;
(3R)-3-{[10-(azetidin-3-yl)-2-(4-methoxyphenyl)[1,2,4]triazolo[1,5-c]quinazolin-5-yl]amino}azepan-2-one;
(3R)-3-[(2-{2-[methanesulfinyl]phenyl}[1,2,4]triazolo[1,5-c]quinazolin-5-yl)amino]azepan-2-one;
(3R)-3-{2-[2-(methanesulfonyl)phenyl][1,2,4]triazolo[1,5-c]quinazolin-5-yl}amino)azepan-2-one;
(3R)-3-{2-[2-(S-methylsulfonimidoyl)phenyl][1,2,4]triazolo[1,5-c]quinazolin-5-yl}amino)azepan-2-one;
(3R)-3-({2-[3-(S-methylsulfonimidoyl)phenyl][1,2,4]triazolo[1,5-c]quinazolin-5-yl}amino)azepan-2-one;
methyl 2-(1-methyl-1H-pyrazol-4-yl)-5-{[(3R)-2-oxoazepan-3-yl]amino}[1,2,4]triazolo[1,5-c]quinazoline-7-carboxylate;
propan-2-yl 2-(4-methoxyphenyl)-5-{[(3R)-2-oxoazepan-3-yl]amino}[1,2,4]triazolo[1,5-c]quinazoline-7-carboxylate;
(3R)-3-{[2-(4-methoxyphenyl)-7-(morpholin-4-yl)[1,2,4]triazolo[1,5-c]quinazolin-5-yl]amino}azepan-2-one;
(3R)-3-{[2-(4-methoxyphenyl)-10-(morpholin-4-yl)[1,2,4]triazolo[1,5-c]quinazolin-5-yl]amino}azepan-2-one;
(6R)-6-{[2-(4-methoxyphenyl)[1,2,4]triazolo[1,5-c]quinazolin-5-yl]amino}-1-methyl-1,4-diazepan-5-one;
ethyl N-[2-(4-methoxyphenyl)[1,2,4]triazolo[1,5-c]quinazolin-5-yl]-D-alaninate;
N-[2-(4-methoxyphenyl)[1,2,4]triazolo[1,5-c]quinazolin-5-yl]-D-alanine;
(3R, R)-3-({2-[3-(S-methylsulfonimidoyl)phenyl][1,2,4]triazolo[1,5-c]quinazolin-5-yl}amino)azepan-2-one;
(3R, S)-3-({2-[3-(S-methylsulfonimidoyl)phenyl][1,2,4]triazolo[1,5-c]quinazolin-5-yl}amino)azepan-2-one;
(3R, R)-3-({2-[2-(S-methylsulfonimidoyl)phenyl][1,2,4]triazolo[1,5-c]quinazolin-5-yl}amino)azepan-2-one;
(3R, S)-3-({2-[2-(S-methylsulfonimidoyl)phenyl][1,2,4]triazolo[1,5-c]quinazolin-5-yl}amino)azepan-2-one;
(2R)—N-ethyl-2-({2-[2-(trifluoromethoxy)phenyl][1,2,4]triazolo[1,5-c]quinazolin-5-yl}amino)butanamide;
(2S)—N-ethyl-2-({2-[2-(trifluoromethoxy)phenyl][1,2,4]triazolo[1,5-c]quinazolin-5-yl}amino)butanamide;
N-cyclohexyl-N2-[2-(4-methoxyphenyl)[1,2,4]triazolo[1,5-c]quinazolin-5-yl]-D-alaninamide;
N-cyclopentyl-N²-[2-(4-methoxyphenyl)[1,2,4]triazolo[1,5-c]quinazolin-5-yl]-D-alaninamide;
(3R)-3-{[7-ethoxy-2-(4-methoxyphenyl)[1,2,4]triazolo[1,5-c]quinazolin-5-yl]amino}azepan-2-one;
(3R)-3-{[7-(2,2-difluoropropoxy)-2-(4-methoxyphenyl)[1,2,4]triazolo[1,5-c]quinazolin-5-yl]amino}azepan-2-one;
(3R)-3-{[7-hydroxy-2-(4-methoxyphenyl)[1,2,4]triazolo[1,5-c]quinazolin-5-yl]amino}azepan-2-one;
(3R)-3-{[2-(4-methoxyphenyl)-7-(3,3,3-trifluoropropoxy)[1,2,4]triazolo[1,5-c]quinazolin-5-yl]amino}azepan-2-one;
4-[5-{[(3R)-2-oxoazepan-3-yl]amino}-7-(trifluoromethyl)[1,2,4]triazolo[1,5-c]quinazolin-2-yl]benzonitrile;
(3R)-3-({2-[4-chloro-2-(trifluoromethoxy)phenyl]-7-(trifluoromethyl)[1,2,4]triazolo[1,5-c]quinazolin-5-yl}amino)azepan-2-one;
(3R)-3-({2-[4-chloro-2-(difluoromethoxy)phenyl]-7-(trifluoromethyl)[1,2,4]triazolo[1,5-c]quinazolin-5-yl}amino)azepan-2-one;
(3R)-3-{[2-(1-ethyl-3-methyl-1H-pyrazol-4-yl)-7-(trifluoromethyl)[1,2,4]triazolo[1,5-c]quinazolin-5-yl]amino}azepan-2-one;
(3R)-3-{[2-(1-ethyl-1H-pyrazol-4-yl)-7-(trifluoromethyl)[1,2,4]triazolo[1,5-c]quinazolin-5-yl]amino}azepan-2-one;
(3R)-3-{[2-(1-cyclopropyl-1H-pyrazol-4-yl)-7-(trifluoromethyl)[1,2,4]triazolo[1,5-c]quinazolin-5-yl]amino}azepan-2-one;
(3R)-3-{[2-(1H-pyrazol-4-yl)-7-(trifluoromethyl)[1,2,4]triazolo[1,5-c]quinazolin-5-yl]amino}azepan-2-one;
(3R)-3-({2-[1-(propan-2-yl)-1H-pyrazol-4-yl]-7-(trifluoromethyl)[1,2,4]triazolo[1,5-c]quinazolin-5-yl}amino)azepan-2-one;
(3R)-3-{[2-(1,3-dimethyl-1H-pyrazol-4-yl)-7-(trifluoromethyl)[1,2,4]triazolo[1,5-c]quinazolin-5-yl]amino}azepan-2-one;
(3S)-3-({7-bromo-2-[4-(methanesulfonyl)phenyl][1,2,4]triazolo[1,5-c]quinazolin-5-yl}amino)azepan-2-one;
(6S)-6-{[7-bromo-2-(4-methoxyphenyl)[1,2,4]triazolo[1,5-c]quinazolin-5-yl]amino}-1,4-oxazepan-5-one;
(3S)-3-({2-[4-bromo-2-(trifluoromethoxy)phenyl][1,2,4]triazolo[1,5-c]quinazolin-5-yl}amino)azepan-2-one;
(2S)-2-({7-bromo-2-[4-chloro-2-(difluoromethoxy)phenyl][1,2,4]triazolo[1,5-c]quinazolin-5-yl}amino)-N-propylbutanamide;
N²-{7-bromo-2-[4-chloro-2-(difluoromethoxy)phenyl][1,2,4]triazolo[1,5-c]quinazolin-5-yl}-N-butyl-D-alaninamide;
(2S)-2-({7-bromo-2-[4-chloro-2-(trifluoromethoxy)phenyl][1,2,4]triazolo[1,5-c]quinazolin-5-yl}amino)-N-propylbutanamide;
N²-{7-bromo-2-[4-chloro-2-(difluoromethoxy)phenyl][1,2,4]triazolo[1,5-c]quinazolin-5-yl}-N-butyl-L-alaninamide;
N²-{7-bromo-2-[4-chloro-2-(trifluoromethoxy)phenyl][1,2,4]triazolo[1,5-c]quinazolin-5-yl}-N-butyl-L-alaninamide;
(2S)-2-{[2-(pyrazin-2-yl)[1,2,4]triazolo[1,5-c]quinazolin-5-yl]amino}butanamide;
(3R)-3-{[2-(pyrazin-2-yl)[1,2,4]triazolo[1,5-c]quinazolin-5-yl]amino}azepan-2-one;
(2S)-2-{[2-(1-methyl-1H-1,2,3-triazol-4-yl)[1,2,4]triazolo[1,5-c]quinazolin-5-yl]amino}butanamide;
(2R)-2-{[2-(pyrazin-2-yl)[1,2,4]triazolo[1,5-c]quinazolin-5-yl]amino}butanamide;
N²-{7-bromo-2-[4-chloro-2-(difluoromethoxy)phenyl][1,2,4]triazolo[1,5-c]quinazolin-5-yl}-N-propyl-D-alaninamide;
N²-{7-bromo-2-[4-chloro-2-(trifluoromethoxy)phenyl][1,2,4]triazolo[1,5-c]quinazolin-5-yl}-N-propyl-D-alaninamide;
(2S)-2-{[7-bromo-2-(1-methyl-1H-pyrazol-4-yl)[1,2,4]triazolo[1,5-c]quinazolin-5-yl]amino}butanamide;
(3S)-3-{[2-(pyrazin-2-yl)[1,2,4]triazolo[1,5-c]quinazolin-5-yl]amino}azepan-2-one;
(2R)-2-{[7-bromo-2-(1-methyl-1H-pyrazol-4-yl)[1,2,4]triazolo[1,5-c]quinazolin-5-yl]amino}butanamide;
(2R)-2-({2-[4-bromo-2-(trifluoromethoxy)phenyl][1,2,4]triazolo[1,5-c]quinazolin-5-yl}amino)butanamide;
(2S)-2-({7-bromo-2-[4-chloro-2-(difluoromethoxy)phenyl][1,2,4]triazolo[1,5-c]quinazolin-5-yl}amino)butanamide;
(6R)-6-{[7-bromo-2-(4-methoxyphenyl)[1,2,4]triazolo[1,5-c]quinazolin-5-yl]amino}-1,4-oxazepan-5-one;
(2R)-2-({2-[4-bromo-2-(difluoromethoxy)phenyl][1,2,4]triazolo[1,5-c]quinazolin-5-yl}amino)butanamide;
(3S)-3-({2-[4-bromo-2-(difluoromethoxy)phenyl][1,2,4]triazolo[1,5-c]quinazolin-5-yl}amino)azepan-2-one;
(2S)-2-({2-[4-bromo-2-(difluoromethoxy)phenyl][1,2,4]triazolo[1,5-c]quinazolin-5-yl}amino)butanamide;
(3R)-3-({2-[4-bromo-2-(difluoromethoxy)phenyl][1,2,4]triazolo[1,5-c]quinazolin-5-yl}amino)azepan-2-one;
(2R)-2-({2-[2-(1,1,2,2-tetrafluoroethoxy)phenyl][1,2,4]triazolo[1,5-c]quinazolin-5-yl}amino)butanamide;
(2S)-2-({2-[2-(1,1,2,2-tetrafluoroethoxy)phenyl][1,2,4]triazolo[1,5-c]quinazolin-5-yl}amino)butanamide;
(3S)-3-({2-[2-(1,1,2,2-tetrafluoroethoxy)phenyl][1,2,4]triazolo[1,5-c]quinazolin-5-yl}amino)azepan-2-one;
(3R)-3-({2-[2-(1,1,2,2-tetrafluoroethoxy)phenyl][1,2,4]triazolo[1,5-c]quinazolin-5-yl}amino)azepan-2-one;
N-butyl-N²-{2-[4-chloro-2-(difluoromethoxy)phenyl][1,2,4]triazolo[1,5-c]quinazolin-5-yl}-L-alaninamide;
4-[5-{[(6R)-5-oxo-1,4-diazepan-6-yl]amino}-7-(trifluoromethyl)[1,2,4]triazolo[1,5-c]quinazolin-2-yl]benzonitrile;
(6R)-6-({2-[4-chloro-2-(trifluoromethoxy)phenyl]-7-(trifluoromethyl)[1,2,4]triazolo[1,5-c]quinazolin-5-yl}amino)-1,4-diazepan-5-one;
(6R)-6-({2-[4-chloro-2-(difluoromethoxy)phenyl]-7-(trifluoromethyl)[1,2,4]triazolo[1,5-c]quinazolin-5-yl}amino)-1,4-diazepan-5-one;
(6R)-6-{[2-(1-ethyl-3-methyl-1H-pyrazol-4-yl)-7-(trifluoromethyl)[1,2,4]triazolo[1,5-c]quinazolin-5-yl]amino}-1,4-diazepan-5-one;
(6R)-6-{[2-(1-ethyl-1H-pyrazol-4-yl)-7-(trifluoromethyl)[1,2,4]triazolo[1,5-c]quinazolin-5-yl]amino}-1,4-diazepan-5-one;
(6R)-6-({2-[4-(methanesulfonyl)phenyl][1,2,4]triazolo[1,5-c]quinazolin-5-yl}amino)-1,4-diazepan-5-one;
(6R)-6-({7-bromo-2-[4-(methanesulfonyl)phenyl][1,2,4]triazolo[1,5-c]quinazolin-5-yl}amino)-1,4-diazepan-5-one;
(6R)-6-({2-[1-(propan-2-yl)-1H-pyrazol-4-yl]-7-(trifluoromethyl)[1,2,4]triazolo[1,5-c]quinazolin-5-yl}amino)-1,4-diazepan-5-one;
4-(7-bromo-5-{[(6R)-5-oxo-1,4-diazepan-6-yl]amino}[1,2,4]triazolo[1,5-c]quinazolin-2-yl)benzonitrile;
4-(5-{[(6R)-5-oxo-1,4-diazepan-6-yl]amino}[1,2,4]triazolo[1,5-c]quinazolin-2-yl)benzonitrile;
(6R)-6-({2-[5-fluoro-2-(trifluoromethoxy)phenyl][1,2,4]triazolo[1,5-c]quinazolin-5-yl}amino)-1,4-diazepan-5-one;
(6R)-6-({2-[2-(trifluoromethoxy)phenyl][1,2,4]triazolo[1,5-c]quinazolin-5-yl}amino)-1,4-diazepan-5-one;
2-(4-methoxyphenyl)-5-{1[(3R)-2-oxoazepan-3-yl]amino}[1,2,4]triazolo[1,5-c]quinazoline-7-carbonitrile;
(3R)-3-({2-[4-methoxy-3-(trifluoromethyl)phenyl][1,2,4]triazolo[1,5-c]quinazolin-5-yl}amino)azepan-2-one;
N²-{7-bromo-2-[4-chloro-2-(trifluoromethoxy)phenyl][1,2,4]triazolo[1,5-c]quinazolin-5-yl}-N-butyl-D-alaninamide;
N-butyl-N²-{2-[4-chloro-2-(difluoromethoxy)phenyl][1,2,4]triazolo[1,5-c]quinazolin-5-yl}-D-alaninamide;
N²-[7-bromo-2-(1-methyl-1H-pyrazol-4-yl)[1,2,4]triazolo[1,5-c]quinazolin-5-yl]-N-butyl-L-alaninamide;
(3R)-3-{[2-(4-methoxyphenyl)-10-(propan-2-yl)[1,2,4]triazolo[1,5-c]quinazolin-5-yl]amino}azepan-2-one;
(6R)-6-{[7-bromo-2-(4-methoxyphenyl)[1,2,4]triazolo[1,5-c]quinazolin-5-yl]amino}-1,4-diazepan-5-one hydrochloride;
4,4-dimethyl-3-{[2-(1-methyl-1H-pyrazol-4-yl)-7-(trifluoromethyl)[1,2,4]triazolo[1,5-c]quinazolin-5-yl]amino}pyrrolidin-2-one;
(−)-4,4-dimethyl-3-{[2-(1-methyl-1H-pyrazol-4-yl)-7-(trifluoromethyl)[1,2,4]triazolo[1,5-c]quinazolin-5-yl]amino}pyrrolidin-2-one;
(+)-4,4-dimethyl-3-{[2-(1-methyl-1H-pyrazol-4-yl)-7-(trifluoromethyl)[1,2,4]triazolo[1,5-c]quinazolin-5-yl]amino}pyrrolidin-2-one;
3-(dimethylamino)-N-methyl-N²-[2-(1-methyl-1H-pyrazol-4-yl)-7-(trifluoromethyl)[1,2,4]triazolo[1,5-c]quinazolin-5-yl]-D-alaninamide;
(6R)-6-{[2-(1-methyl-1H-pyrazol-4-yl)-7-(trifluoromethyl)[1,2,4]triazolo[1,5-c]quinazolin-5-yl]amino}-1,4-oxazepan-5-one;
(3R)-1-methyl-3-{[2-(1-methyl-1H-pyrazol-4-yl)-7-(trifluoromethyl)[1,2,4]triazolo[1,5-c]quinazolin-5-yl]amino}pyrrolidin-2-one;
(3R)-3-{[2-(1,5-dimethyl-1H-pyrazol-4-yl)-7-(trifluoromethyl)[1,2,4]triazolo[1,5-c]quinazolin-5-yl]amino}azepan-2-one;
(3R)-3-{[2-(1-methyl-1H-pyrazol-3-yl)-7-(trifluoromethyl)[1,2,4]triazolo[1,5-c]quinazolin-5-yl]amino}azepan-2-one;
(3R)-3-{2-[1-(difluoromethyl)-1H-pyrazol-4-yl]-7-(trifluoromethyl)[1,2,4]triazolo[1,5-c]quinazolin-5-yl}amino)azepan-2-one;
(3R)-3-{[2-(1H-pyrazol-3-yl)-7-(trifluoromethyl)[1,2,4]triazolo[1,5-c]quinazolin-5-yl]amino}azepan-2-one;
(3R)-3-{[2-(1-ethyl-3,5-dimethyl-1H-pyrazol-4-yl)-7-(trifluoromethyl)[1,2,4]triazolo[1,5-c]quinazolin-5-yl]amino}azepan-2-one;
(3R)-1-methyl-3-{[2-(1-methyl-1H-pyrazol-4-yl)-7-(trifluoromethyl)[1,2,4]triazolo[1,5-c]quinazolin-5-yl]amino}azepan-2-one;
(3R)-3-{[2-(1,3-dimethyl-1H-pyrazol-5-yl)-7-(trifluoromethyl)[1,2,4]triazolo[1,5-c]quinazolin-5-yl]amino}azepan-2-one;
(3R)-3-{[2-(1-cyclobutyl-1H-pyrazol-4-yl)-7-(trifluoromethyl)[1,2,4]triazolo[1,5-c]quinazolin-5-yl]amino}azepan-2-one;
(3R)-3-{2-[1-(cyclopropylmethyl)-1H-pyrazol-4-yl]-7-(trifluoromethyl)[1,2,4]triazolo[1,5-c]quinazolin-5-yl}amino)azepan-2-one;
N-(3-fluoropyridin-4-yl)-N-(4-{2-[2-(trifluoromethyl)phenyl]acetamido}pyridin-2-yl)acetamide;
(3R)-3-{2-[1-(cyclobutylmethyl)-1H-pyrazol-4-yl]-7-(trifluoromethyl)[1,2,4]triazolo[1,5-c]quinazolin-5-yl}amino)azepan-2-one;
(3R)-3-{[2-(1-cyclopropyl-3,5-dimethyl-1H-pyrazol-4-yl)-7-(trifluoromethyl)[1,2,4]triazolo[1,5-c]quinazolin-5-yl]amino}azepan-2-one;
(3R)-3-{[2-(3-methyl-1H-pyrazol-4-yl)-7-(trifluoromethyl)[1,2,4]triazolo[1,5-c]quinazolin-5-yl]amino}azepan-2-one;
(3R)-3-{[2-(4-cyclopropyl-1,3-thiazol-2-yl)-7-(trifluoromethyl)[1,2,4]triazolo[1,5-c]quinazolin-5-yl]amino}azepan-2-one;
(3R)-3-({2-[3-methyl-1-(propan-2-yl)-1H-pyrazol-4-yl]-7-(trifluoromethyl)[1,2,4]triazolo[1,5-c]quinazolin-5-yl}amino)azepan-2-one;
(3R)-3-{[2-(1-cyclopropyl-3-ethyl-1H-pyrazol-4-yl)-7-(trifluoromethyl)[1,2,4]triazolo[1,5-c]quinazolin-5-yl]amino}azepan-2-one;
(3R)-3-{[7-(trifluoromethyl)-2-(1,3,5-trimethyl-1H-pyrazol-4-yl)[1,2,4]triazolo[1,5-c]quinazolin-5-yl]amino}azepan-2-one;
N-butyl-N²-[2-(1-methyl-1H-pyrazol-4-yl)-7-(trifluoromethyl)[1,2,4]triazolo[1,5-c]quinazolin-5-yl]-D-alaninamide;
(3R)-3-{[7-methoxy-2-(1-methyl-1H-pyrazol-4-yl)[1,2,4]triazolo[1,5-c]quinazolin-5-yl]amino}azepan-2-one;
(3R)-3-{[2-(1-ethyl-3-methyl-1H-pyrazol-4-yl)-7-methoxy[1,2,4]triazolo[1,5-c]quinazolin-5-yl]amino}azepan-2-one;
(3R)-3-{[2-(1-ethyl-1H-pyrazol-4-yl)-7-methoxy[1,2,4]triazolo[1,5-c]quinazolin-5-yl]amino}azepan-2-one;
(3R)-3-{[7-methoxy-2-(1H-pyrazol-4-yl)[1,2,4]triazolo[1,5-c]quinazolin-5-yl]amino}azepan-2-one;
(3R)-3-{[7-methoxy-2-(1-methyl-1H-1,2,3-triazol-4-yl)[1,2,4]triazolo[1,5-c]quinazolin-5-yl]amino}azepan-2-one;
(3R)-3-{[2-(1-cyclopropyl-1H-pyrazol-4-yl)-7-methoxy[1,2,4]triazolo[1,5-c]quinazolin-5-yl]amino}azepan-2-one;
(3R)-3-{[2-(1-cyclobutyl-1H-pyrazol-4-yl)-7-methoxy[1,2,4]triazolo[1,5-c]quinazolin-5-yl]amino}azepan-2-one;
(3R)-3-({2-[1-(propan-2-yl)-1H-pyrazol-4-yl]-7-(trifluoromethoxy)[1,2,4]triazolo[1,5-c]quinazolin-5-yl}amino)azepan-2-one;
5-{[(3R)-2-oxoazepan-3-yl]amino}-2-[1-(propan-2-yl)-1H-pyrazol-4-yl][1,2,4]triazolo[1,5-c]quinazoline-7-carbonitrile;
2-(1-cyclopropyl-1H-pyrazol-4-yl)-5-{[(3R)-2-oxoazepan-3-yl]amino}[1,2,4]triazolo[1,5-c]quinazoline-7-carbonitrile;
2-(1-cyclobutyl-1H-pyrazol-4-yl)-5-{[(3R)-2-oxoazepan-3-yl]amino}[1,2,4]triazolo[1,5-c]quinazoline-7-carbonitrile;
2-(1-ethyl-3-methyl-1H-pyrazol-4-yl)-5-{[(3R)-2-oxoazepan-3-yl]amino}[1,2,4]triazolo[1,5-c]quinazoline-7-carbonitrile;
2-[1-(difluoromethyl)-1H-pyrazol-4-yl]-5-{[(3R)-2-oxoazepan-3-yl]amino}[1,2,4]triazolo[1,5-c]quinazoline-7-carbonitrile;
5-{[(3R)-2-oxoazepan-3-yl]amino}-2-(1H-pyrazol-4-yl)[1,2,4]triazolo[1,5-c]quinazoline-7-carbonitrile;
(3R)-3-{[2-(4-methoxyphenyl)-7-(methylsulfanyl)[1,2,4]triazolo[1,5-c]quinazolin-5-yl]amino}azepan-2-one;
(3R)-3-({2-(4-methoxyphenyl)-7-[(propan-2-yl)sulfanyl][1,2,4]triazolo[1,5-c]quinazolin-5-yl}amino)azepan-2-one;
(3R)-3-({2-(1-methyl-1H-pyrazol-4-yl)-7-[(propan-2-yl)sulfanyl][1,2,4]triazolo[1,5-c]quinazolin-5-yl}amino)azepan-2-one;
(3R)-3-{[7-(ethylsulfanyl)-2-(1-methyl-1H-pyrazol-4-yl)[1,2,4]triazolo[1,5-c]quinazolin-5-yl]amino}azepan-2-one;
(3R)-3-{[7-(ethylsulfanyl)-2-(4-methoxyphenyl)[1,2,4]triazolo[1,5-c]quinazolin-5-yl]amino}azepan-2-one;
(3R)-3-({7-(methylsulfanyl)-2-[1-(propan-2-yl)-1H-pyrazol-4-yl][1,2,4]triazolo[1,5-c]quinazolin-5-yl}amino)azepan-2-one;
(3R)-3-{[2-(1-ethyl-3-methyl-1H-pyrazol-4-yl)-7-(methylsulfanyl)[1,2,4]triazolo[1,5-c]quinazolin-5-yl]amino}azepan-2-one;
(2R)-2-{[7-bromo-2-(4-methoxyphenyl)[1,2,4]triazolo[1,5-c]quinazolin-5-yl]amino}-1-(morpholin-4-yl)propan-1-one;
N-[2-(dimethylamino)ethyl]-N²-{2-[4-(methanesulfonyl)phenyl][1,2,4]triazolo[1,5-c]quinazolin-5-yl}-D-alaninamide;
N²-{2-[5-chloro-2-(trifluoromethoxy)phenyl][1,2,4]triazolo[1,5-c]quinazolin-5-yl}-N-[2-(dimethylamino)ethyl]-D-alaninamide;
N-[2-(dimethylamino)ethyl]-N²-[2-(1-methyl-1H-pyrazol-4-yl)[1,2,4]triazolo[1,5-c]quinazolin-5-yl]-D-alaninamide;
(2R)-2-{[2-(4-methoxyphenyl)[1,2,4]triazolo[1,5-c]quinazolin-5-yl]amino}-1-(4-methylpiperazin-1-yl)propan-1-one;
(2R)-2-{[2-(4-methoxyphenyl)[1,2,4]triazolo[1,5-c]quinazolin-5-yl]amino}-1-(morpholin-4-yl)propan-1-one;
N²-[2-(4-methoxyphenyl)[1,2,4]triazolo[1,5-c]quinazolin-5-yl]-N-[2-(morpholin-4-yl)ethyl]-D-alaninamide;
N²-[7-bromo-2-(4-methoxyphenyl)[1,2,4]triazolo[1,5-c]quinazolin-5-yl]-N-[2-(4-methylpiperazin-1-yl)ethyl]-D-alaninamide;
N²-[7-bromo-2-(4-methoxyphenyl)[1,2,4]triazolo[1,5-c]quinazolin-5-yl]-N-[2-(morpholin-4-yl)ethyl]-D-alaninamide;
N²-[7-bromo-2-(1-methyl-1H-pyrazol-4-yl)[1,2,4]triazolo[1,5-c]quinazolin-5-yl]-N-[2-(morpholin-4-yl)ethyl]-D-alaninamide;
N²-[7-bromo-2-(1-methyl-1H-pyrazol-4-yl)[1,2,4]triazolo[1,5-c]quinazolin-5-yl]-N-[2-(4-methylpiperazin-1-yl)ethyl]-D-alaninamide;
N-(2-amino-2-methylpropyl)-N²-[2-(4-methoxyphenyl)[1,2,4]triazolo[1,5-c]quinazolin-5-yl]-D-alaninamide;
(2R)-2-{[7-bromo-2-(1-methyl-1H-pyrazol-4-yl)[1,2,4]triazolo[1,5-c]quinazolin-5-yl]amino}-1-(morpholin-4-yl)propan-1-one;
(2R)-2-{[7-bromo-2-(1-methyl-1H-pyrazol-4-yl)[1,2,4]triazolo[1,5-c]quinazolin-5-yl]amino}-1-(4-methylpiperazin-1-yl)propan-1-one;
N-[2-(dimethylamino)ethyl]-N²-[2-(4-methoxyphenyl)[1,2,4]triazolo[1,5-c]quinazolin-5-yl]-D-alaninamide;
(2R)-2-{[7-bromo-2-(4-methoxyphenyl)[1,2,4]triazolo[1,5-c]quinazolin-5-yl]amino}-1-(4-methylpiperazin-1-yl)propan-1-one;
N-(2-amino-2-methylpropyl)-N²-[7-bromo-2-(4-methoxyphenyl)[1,2,4]triazolo[1,5-c]quinazolin-5-yl]-D-alaninamide;
N-(2-amino-2-methylpropyl)-N²-[7-bromo-2-(1-methyl-1H-pyrazol-4-yl)[1,2,4]triazolo[1,5-c]quinazolin-5-yl]-D-alaninamide;
formic acid N-[2-(dimethylamino)ethyl]-N²-{2-[2-(trifluoromethoxy)phenyl][1,2,4]triazolo[1,5-c]quinazolin-5-yl}-D-alaninamide (1/1);
N-[2-(dimethylamino)ethyl]-N²-{2-[2-(trifluoromethoxy)phenyl][1,2,4]triazolo[1,5-c]quinazolin-5-yl}-D-alaninamide;
(2R)-2-{[2-(1-methyl-1H-1,2,3-triazol-4-yl)[1,2,4]triazolo[1,5-c]quinazolin-5-yl]amino}butanamide;
N²-[7-bromo-2-(1-methyl-1H-pyrazol-4-yl)[1,2,4]triazolo[1,5-c]quinazolin-5-yl]-N-butyl-D-alaninamide;
2-[1-(6-{[(3S)-1-azabicyclo[2.2.2]octan-3-yl]amino}-1-[3-(trifluoromethyl)phenyl]-1H-pyrazolo[3,4-d]pyrimidin-4-yl)piperidin-4-yl]propan-2-ol;
N²-[7-bromo-2-(1-methyl-1H-pyrazol-4-yl)[1,2,4]triazolo[1,5-c]quinazolin-5-yl]-N-propyl-D-alaninamide;
(2R)-2-({7-bromo-2-[4-chloro-2-(difluoromethoxy)phenyl][1,2,4]triazolo[1,5-c]quinazolin-5-yl}amino)-N-propylbutanamide;
(2R)-2-({7-bromo-2-[4-chloro-2-(difluoromethoxy)phenyl][1,2,4]triazolo[1,5-c]quinazolin-5-yl}amino)-N-ethylbutanamide;
(2S)-2-({7-bromo-2-[4-chloro-2-(difluoromethoxy)phenyl][1,2,4]triazolo[1,5-c]quinazolin-5-yl}amino)-N-ethylbutanamide;
(2R)-2-{[7-bromo-2-(1-methyl-1H-pyrazol-5-yl)[1,2,4]triazolo[1,5-c]quinazolin-5-yl]amino}butanamide;
(2S)-2-{[7-bromo-2-(1-methyl-1H-pyrazol-5-yl)[1,2,4]triazolo[1,5-c]quinazolin-5-yl]amino}butanamide;
N²-{7-bromo-2-[4-chloro-2-(difluoromethoxy)phenyl][1,2,4]triazolo[1,5-c]quinazolin-5-yl}-N-propyl-L-alaninamide;
(2S)-2-({7-bromo-2-[4-chloro-2-(difluoromethoxy)phenyl][1,2,4]triazolo[1,5-c]quinazolin-5-yl}amino)-N-butylbutanamide;
2-fluoro-3-(5-methylpyridin-2-yl)-N-[(1R)-1-(6-methylpyridin-3-yl)ethyl]benzamide;
(2S)-2-{[2-(1-methyl-1H-pyrazol-5-yl)-7-(trifluoromethyl)[1,2,4]triazolo[1,5-c]quinazolin-5-yl]amino}butanamide;
(2R)-2-{[2-(1-methyl-1H-pyrazol-5-yl)-7-(trifluoromethyl)[1,2,4]triazolo[1,5-c]quinazolin-5-yl]amino}butanamide;
(3S)-3-{[2-(1-methyl-1H-pyrazol-5-yl)-7-(trifluoromethyl)[1,2,4]triazolo[1,5-c]quinazolin-5-yl]amino}azepan-2-one;
(3R)-3-{[2-(1-methyl-1H-pyrazol-5-yl)-7-(trifluoromethyl)[1,2,4]triazolo[1,5-c]quinazolin-5-yl]amino}azepan-2-one;
N²-[7-bromo-2-(4-methoxyphenyl)[1,2,4]triazolo[1,5-c]quinazolin-5-yl]-N-[3-(dimethylamino)propyl]-D-alaninamide;
N²-[7-bromo-2-(1-methyl-1H-pyrazol-4-yl)[1,2,4]triazolo[1,5-c]quinazolin-5-yl]-N-[3-(dimethylamino)propyl]-D-alaninamide;
N²-{7-bromo-2-[4-chloro-2-(trifluoromethoxy)phenyl][1,2,4]triazolo[1,5-c]quinazolin-5-yl}-N-propyl-L-alaninamide;
(2R)-2-({7-bromo-2-[4-chloro-2-(trifluoromethoxy)phenyl][1,2,4]triazolo[1,5-c]quinazolin-5-yl}amino)-N-propylbutanamide;
(2S)-2-({7-bromo-2-[4-chloro-2-(trifluoromethoxy)phenyl][1,2,4]triazolo[1,5-c]quinazolin-5-yl}amino)-N-butylbutanamide;
(2R)-2-({7-bromo-2-[4-chloro-2-(trifluoromethoxy)phenyl][1,2,4]triazolo[1,5-c]quinazolin-5-yl}amino)-N-butylbutanamide;
(3R)-3-{[7-bromo-2-(1,3-dimethyl-1H-pyrazol-4-yl)[1,2,4]triazolo[1,5-c]quinazolin-5-yl]amino}azepan-2-one;
(3R)-3-{[7-chloro-2-(1-cyclopropyl-1H-pyrazol-4-yl)[1,2,4]triazolo[1,5-c]quinazolin-5-yl]amino}azepan-2-one;
(3R)-3-({7-chloro-2-[1-(propan-2-yl)-1H-pyrazol-4-yl][1,2,4]triazolo[1,5-c]quinazolin-5-yl}amino)azepan-2-one;
(3R)-3-{[7-chloro-2-(1-ethyl-1H-pyrazol-4-yl)[1,2,4]triazolo[1,5-c]quinazolin-5-yl]amino}azepan-2-one;
(3R)-3-{[7-chloro-2-(1-ethyl-3-methyl-1H-pyrazol-4-yl)[1,2,4]triazolo[1,5-c]quinazolin-5-yl]amino}azepan-2-one;
(3R)-3-({7-cyclopropyl-2-[1-(propan-2-yl)-1H-pyrazol-4-yl][1,2,4]triazolo[1,5-c]quinazolin-5-yl}amino)azepan-2-one;
(3R)-3-{[7-chloro-2-(1,3-dimethyl-1H-pyrazol-4-yl)[1,2,4]triazolo[1,5-c]quinazolin-5-yl]amino}azepan-2-one;
(3R)-3-({7-bromo-2-[1-(propan-2-yl)-1H-pyrazol-4-yl][1,2,4]triazolo[1,5-c]quinazolin-5-yl}amino)azepan-2-one;
(3R)-3-{[7-bromo-2-(1,5-dimethyl-1H-pyrazol-4-yl)[1,2,4]triazolo[1,5-c]quinazolin-5-yl]amino}azepan-2-one;
(3R)-3-{[7-bromo-2-(1-cyclobutyl-1H-pyrazol-4-yl)[1,2,4]triazolo[1,5-c]quinazolin-5-yl]amino}azepan-2-one;
(3R)-3-{[7-bromo-2-(1-ethyl-3-methyl-1H-pyrazol-4-yl)[1,2,4]triazolo[1,5-c]quinazolin-5-yl]amino}azepan-2-one;
(3R)-3-({7-bromo-2-[1-(cyclopropylmethyl)-1H-pyrazol-4-yl][1,2,4]triazolo[1,5-c]quinazolin-5-yl}amino)azepan-2-one;
(3R)-3-({7-bromo-2-[1-(difluoromethyl)-1H-pyrazol-4-yl][1,2,4]triazolo[1,5-c]quinazolin-5-yl}amino)azepan-2-one;
N²-(4-amino-5-benzoyl-1,3-thiazol-2-yl)-N²-(3-methylphenyl)alaninamide;
N-[(3S)-1-benzoyl-3-(4-chloro-3-methylphenyl)pyrrolidin-3-yl]-N′-isoquinolin-6-ylurea;
(3R)-3-{[7-bromo-2-(4-chlorophenyl)[1,2,4]triazolo[1,5-c]quinazolin-5-yl]amino}azepan-2-one;
(3R)-3-{[7-bromo-2-(1-ethyl-1H-pyrazol-4-yl)[1,2,4]triazolo[1,5-c]quinazolin-5-yl]amino}azepan-2-one;
(3R)-3-({7-bromo-2-[1-(cyclobutylmethyl)-1H-pyrazol-4-yl][1,2,4]triazolo[1,5-c]quinazolin-5-yl}amino)azepan-2-one;
(3R)-3-{[2-(4-chlorophenyl)-7-(trifluoromethyl)[1,2,4]triazolo[1,5-c]quinazolin-5-yl]amino}azepan-2-one;
2-bromo-4-chloro-6-fluoro-N-{(2R)-1-[(4-iodo-6-methoxypyridin-3-yl)oxy]propan-2-yl}aniline;
N-[4-({(1r,4r)-4-[(8-cyanoquinolin-4-yl)oxy]cyclohexyl}carbamoyl)phenyl]-14-({2-[(3RS)-2,6-dioxopiperidin-3-yl]-1,3-dioxo-2,3-dihydro-1H-isoindol-4-yl}oxy)-3,6,9,12-tetraoxatetradecan-1-amide;
benzyl (6R)-6-({7-fluoro-2-[1-(propan-2-yl)-1H-pyrazol-4-yl][1,2,4]triazolo[1,5-c]quinazolin-5-yl}amino)-5-oxo-1,4-diazepane-1-carboxylate;
(6S)-6-{[7-bromo-2-(1-methyl-1H-pyrazol-4-yl)[1,2,4]triazolo[1,5-c]quinazolin-5-yl]amino}-1,4-thiazepan-5-one;
(6S)-6-({2-[1-(propan-2-yl)-1H-pyrazol-4-yl]-7-(trifluoromethyl)[1,2,4]triazolo[1,5-c]quinazolin-5-yl}amino)-1,4-thiazepan-5-one;
(6S)-6-{[7-bromo-2-(4-methoxyphenyl)[1,2,4]triazolo[1,5-c]quinazolin-5-yl]amino}-1,4-thiazepan-5-one;
(6R)-6-{[7-bromo-2-(4-methoxyphenyl)[1,2,4]triazolo[1,5-c]quinazolin-5-yl]amino}-1,4-thiazepan-5-one;
(6R)-6-{[7-bromo-2-(1-methyl-1H-pyrazol-4-yl)[1,2,4]triazolo[1,5-c]quinazolin-5-yl]amino}-1,4-thiazepan-5-one;
(3R)-3-{[7-(methanesulfonyl)-2-(4-methoxyphenyl)[1,2,4]triazolo[1,5-c]quinazolin-5-yl]amino}azepan-2-one;
(3R)-3-{[2-(4-methoxyphenyl)-7-(propane-2-sulfonyl)[1,2,4]triazolo[1,5-c]quinazolin-5-yl]amino}azepan-2-one;
(3R)-3-{[7-(ethanesulfonyl)-2-(4-methoxyphenyl)[1,2,4]triazolo[1,5-c]quinazolin-5-yl]amino}azepan-2-one;
(3R)-3-{[2-(1-methyl-1H-pyrazol-4-yl)-7-(propane-2-sulfonyl)[1,2,4]triazolo[1,5-c]quinazolin-5-yl]amino}azepan-2-one;
(3R)-3-({7-(methanesulfonyl)-2-[1-(propan-2-yl)-1H-pyrazol-4-yl][1,2,4]triazolo[1,5-c]quinazolin-5-yl}amino)azepan-2-one;
(3R)-3-{[2-(1-ethyl-3-methyl-1H-pyrazol-4-yl)-7-(methanesulfonyl)[1,2,4]triazolo[1,5-c]quinazolin-5-yl]amino}azepan-2-one;
(6R)-6-{[7-bromo-2-(1-methyl-1H-pyrazol-4-yl)[1,2,4]triazolo[1,5-c]quinazolin-5-yl]amino}-1λ⁶,4-thiazepane-1,1,5-trione;
(1R*,6R)-6-{[7-bromo-2-(1-methyl-1H-pyrazol-4-yl)[1,2,4]triazolo[1,5-c]quinazolin-5-yl]amino}-1λ⁴,4-thiazepane-1,5-dione;
(1R*,6R)-6-{[7-bromo-2-(1-methyl-1H-pyrazol-4-yl)[1,2,4]triazolo[1,5-c]quinazolin-5-yl]amino}-1λ⁴,4-thiazepane-1,5-dione;
(6S)-6-{[7-bromo-2-(4-methoxyphenyl)[1,2,4]triazolo[1,5-c]quinazolin-5-yl]amino}-1λ⁶,4-thiazepane-1,1,5-trione;
(6S)-6-({2-[1-(propan-2-yl)-1H-pyrazol-4-yl]-7-(trifluoromethyl)[1,2,4]triazolo[1,5-c]quinazolin-5-yl}amino)-1λ⁶,4-thiazepane-1,1,5-trione;
(3R)-3-{[7-(S-methanesulfonimidoyl)-2-(4-methoxyphenyl)[1,2,4]triazolo[1,5-c]quinazolin-5-yl]amino}azepan-2-one;
(3R)-3-{[7-(S-methanesulfonimidoyl)-2-(4-methoxyphenyl)[1,2,4]triazolo[1,5-c]quinazolin-5-yl]amino}azepan-2-one;
(3R)-3-{[7-(S-methanesulfonimidoyl)-2-(4-methoxyphenyl)[1,2,4]triazolo[1,5-c]quinazolin-5-yl]amino}azepan-2-one;
(3R,4S)-4-[(4-fluorophenyl)methyl]-1-[5-({1[(pyridin-2-yl)methyl]amino}methyl)pyrimidin-2-yl]-3-(trifluoromethyl)pyrrolidin-3-ol;
(6R)-6-{[7-bromo-2-(1-methyl-1H-pyrazol-4-yl)[1,2,4]triazolo[1,5-c]quinazolin-5-yl]amino}-1-imino-1λ⁶,4-thiazepane-1,5-dione;
(3R)-3-{[7-(methanesulfonyl)-2-(1-methyl-H-pyrazol-4-yl)[1,2,4]triazolo[1,5-c]quinazolin-5-yl]amino}azepan-2-one;
(3R)-3-{[7-(2-hydroxypropan-2-yl)-2-(4-methoxyphenyl)[1,2,4]triazolo[1,5-c]quinazolin-5-yl]amino}azepan-2-one;
(3R)-3-{[7-(hydroxymethyl)-2-(4-methoxyphenyl)[1,2,4]triazolo[1,5-c]quinazolin-5-yl]amino}azepan-2-one;
(3R)-3-{[7-(2-hydroxypropan-2-yl)-2-(1-methyl-1H-pyrazol-4-yl)[1,2,4]triazolo[1,5-c]quinazolin-5-yl]amino}azepan-2-one;
(3R)-3-{[10-(hydroxymethyl)-2-(4-methoxyphenyl)[1,2,4]triazolo[1,5-c]quinazolin-5-yl]amino}azepan-2-one;
(3R)-3-({2-(4-methoxyphenyl)-7-[(methylsulfanyl)methyl][1,2,4]triazolo[1,5-c]quinazolin-5-yl}amino)azepan-2-one;
3-[3-({4-[4-(3-chlorophenoxy)piperidin-1-yl]-3-cyano-1-methyl-2-oxo-1,2-dihydroquinolin-7-yl}oxy)propoxy]-N-[2-({2-[(3S)-2,6-dioxopiperidin-3-yl]-1,3-dioxo-2,3-dihydro-1H-isoindol-5-yl}oxy)ethyl]propanamide;
2-[(3R,4S)-4-[(4-fluorophenyl)methyl]-3-hydroxy-3-(trifluoromethyl)pyrrolidin-1-yl]-N-(pyridin-4-yl)pyrimidine-4-carboxamide;
ethyl N-[7-bromo-2-(1-methyl-1H-pyrazol-4-yl)[1,2,4]triazolo[1,5-c]quinazolin-5-yl]-D-alaninate;
methyl N-[7-bromo-2-(4-methoxyphenyl)[1,2,4]triazolo[1,5-c]quinazolin-5-yl]-D-valinate;
methyl N-[7-bromo-2-(1-methyl-1H-pyrazol-4-yl)[1,2,4]triazolo[1,5-c]quinazolin-5-yl]-D-valinate;
N-[2-(1-methyl-1H-pyrazol-4-yl)-7-(trifluoromethyl)[1,2,4]triazolo[1,5-c]quinazolin-5-yl]-D-alanine;
N-[7-bromo-2-(4-methoxyphenyl)[1,2,4]triazolo[1,5-c]quinazolin-5-yl]-D-alanine;
N-[7-bromo-2-(1-methyl-1H-pyrazol-4-yl)[1,2,4]triazolo[1,5-c]quinazolin-5-yl]-D-alanine;
N-[7-bromo-2-(4-methoxyphenyl)[1,2,4]triazolo[1,5-c]quinazolin-5-yl]-D-valine;
N-[7-bromo-2-(1-methyl-1H-pyrazol-4-yl)[1,2,4]triazolo[1,5-c]quinazolin-5-yl]-D-valine;
(2R)-2-{[2-(1-methyl-1H-pyrazol-4-yl)-7-(trifluoromethyl)[1,2,4]triazolo[1,5-c]quinazolin-5-yl]amino}-1-(morpholin-4-yl)propan-1-one;
(2R)-1-(4-methylpiperazin-1-yl)-2-{[2-(1-methyl-1H-pyrazol-4-yl)-7-(trifluoromethyl)[1,2,4]triazolo[1,5-c]quinazolin-5-yl]amino}propan-1-one;
(2R)-1-[3-(dimethylamino)pyrrolidin-1-yl]-2-{[2-(1-methyl-1H-pyrazol-4-yl)-7-(trifluoromethyl)[1,2,4]triazolo[1,5-c]quinazolin-5-yl]amino}propan-1-one;
N-[2-(dimethylamino)ethyl]-N-methyl-N²-[2-(1-methyl-1H-pyrazol-4-yl)-7-(trifluoromethyl)[1,2,4]triazolo[1,5-c]quinazolin-5-yl]-D-alaninamide;
N-(2-methoxyethyl)-N-methyl-N²-[2-(1-methyl-1H-pyrazol-4-yl)-7-(trifluoromethyl)[1,2,4]triazolo[1,5-c]quinazolin-5-yl]-D-alaninamide;
N-[2-(4-methylpiperazin-1-yl)ethyl]-N²-[2-(1-methyl-1H-pyrazol-4-yl)-7-(trifluoromethyl)[1,2,4]triazolo[1,5-c]quinazolin-5-yl]-D-alaninamide;
(2R)-2-{[2-(1-methyl-1H-pyrazol-4-yl)-7-(trifluoromethyl)[1,2,4]triazolo[1,5-c]quinazolin-5-yl]amino}-1-(2-oxa-6-azaspiro[3.3]heptan-6-yl)propan-1-one;
N²-[2-(4-methoxyphenyl)[1,2,4]triazolo[1,5-c]quinazolin-5-yl]-N-[2-(4-methylpiperazin-1-yl)ethyl]-D-alaninamide;
tert-butyl 4-{N-[2-(4-methoxyphenyl)[1,2,4]triazolo[1,5-c]quinazolin-5-yl]-D-alanyl}piperazine-1-carboxylate;
tert-butyl 4-{N-[7-bromo-2-(4-methoxyphenyl)[1,2,4]triazolo[1,5-c]quinazolin-5-yl]-D-alanyl}piperazine-1-carboxylate;
tert-butyl 4-[2-({N-[2-(4-methoxyphenyl)[1,2,4]triazolo[1,5-c]quinazolin-5-yl]-D-alanyl}amino)ethyl]piperazine-1-carboxylate;
N²-[7-bromo-2-(4-methoxyphenyl)[1,2,4]triazolo[1,5-c]quinazolin-5-yl]-N-(2-methoxyethyl)-D-alaninamide;
tert-butyl 4-{N-[7-bromo-2-(1-methyl-1H-pyrazol-4-yl)[1,2,4]triazolo[1,5-c]quinazolin-5-yl]-D-alanyl}piperazine-1-carboxylate;
tert-butyl [2-({N-[7-bromo-2-(4-methoxyphenyl)[1,2,4]triazolo[1,5-c]quinazolin-5-yl]-D-alanyl}amino)ethyl]methylcarbamate;
tert-butyl [2-({N-[7-bromo-2-(1-methyl-1H-pyrazol-4-yl)[1,2,4]triazolo[1,5-c]quinazolin-5-yl]-D-alanyl}amino)ethyl]methylcarbamate;
tert-butyl [2-({N-[2-(4-methoxyphenyl)[1,2,4]triazolo[1,5-c]quinazolin-5-yl]-D-alanyl}amino)ethyl]methylcarbamate;
tert-butyl 4-[2-({N-[7-bromo-2-(4-methoxyphenyl)[1,2,4]triazolo[1,5-c]quinazolin-5-yl]-D-alanyl}amino)ethyl]piperazine-1-carboxylate;
tert-butyl 4-[2-({N-[7-bromo-2-(1-methyl-1H-pyrazol-4-yl)[1,2,4]triazolo[1,5-c]quinazolin-5-yl]-D-alanyl}amino)ethyl]piperazine-1-carboxylate;
methyl 4-{N-[7-bromo-2-(1-methyl-1H-pyrazol-4-yl)[1,2,4]triazolo[1,5-c]quinazolin-5-yl]-D-alanyl}piperazine-1-carboxylate;
N⁶-(propan-2-yl)lysyl-N-[(5aS,11S,14S,17S,20S,23R,28R,31S,33aS)-31-[(2S)-butan-2-yl]-17-(3-carbamimidamidopropyl)-28-1{[(1S,2S)-1-carboxy-2-methylbutyl]carbamoyl}-11-[(3-carboxyphenyl)methyl]-14,20-bis(hydroxymethyl)-27,27-dimethyl-5,10,13,16,19,22,30,33-octaoxooctacosahydro-1H,5H,10H-dipyrrolo[2,1-j:2′,1′-m][1,2,5,8,11,14,17,20,23,26]dithiaoctaazacyclononacosin-23-yl]-L-isoleucinamide;
N²-[7-bromo-2-(4-methoxyphenyl)[1,2,4]triazolo[1,5-c]quinazolin-5-yl]-N-[2-(piperidin-1-yl)ethyl]-D-alaninamide;
N²-[7-bromo-2-(1-methyl-1H-pyrazol-4-yl)[1,2,4]triazolo[1,5-c]quinazolin-5-yl]-N-[2-(piperidin-1-yl)ethyl]-D-alaninamide;
N²-[2-(4-methoxyphenyl)[1,2,4]triazolo[1,5-c]quinazolin-5-yl]-N-[2-(piperidin-1-yl)ethyl]-D-alaninamide;
2-methyl-5-oxo-N-[(pyridin-2-yl)methyl]-2,3-dihydro-5H-[1,3]thiazolo[3,2-a]pyrimidine-6-carboxamide;
5-(4-{4-chloro-3-[(7,8,9,10-tetrahydro[1,2,4]triazolo[3,4-a]phthalazin-6-yl)oxy]phenyl}piperazin-1-yl)-2-[(3R)-2,6-dioxopiperidin-3-yl]-1H-isoindole-1,3(2H)-dione;
(2R)-2-{[7-bromo-2-(1-methyl-1H-pyrazol-4-yl)[1,2,4]triazolo[1,5-c]quinazolin-5-yl]amino}-1-[(1S,4S)-5-methyl-2,5-diazabicyclo[2.2.1]heptan-2-yl]propan-1-one;
(2R)-2-{[7-bromo-2-(4-methoxyphenyl)[1,2,4]triazolo[1,5-c]quinazolin-5-yl]amino}-1-[(1S,4S)-5-methyl-2,5-diazabicyclo[2.2.1]heptan-2-yl]propan-1-one;
(3R)-1-[(1H-imidazol-2-yl)methyl]-2′-(quinolin-3-yl)-4′H,6′H-spiro[pyrrolidine-3,5′-pyrrolo[1,2-b]pyrazole];
(2R)-2-{[7-bromo-2-(4-methoxyphenyl)[1,2,4]triazolo[1,5-c]quinazolin-5-yl]amino}-1-[(1R,4R)-5-methyl-2,5-diazabicyclo[2.2.1]heptan-2-yl]propan-1-one;
N²-[7-bromo-2-(4-methoxyphenyl)[1,2,4]triazolo[1,5-c]quinazolin-5-yl]-N-(2-hydroxy-2-methylpropyl)-D-alaninamide;
N²-[7-bromo-2-(1-methyl-1H-pyrazol-4-yl)[1,2,4]triazolo[1,5-c]quinazolin-5-yl]-N-(2-hydroxy-2-methylpropyl)-D-alaninamide;
(2R*)—N-(4-ethylphenyl)-2-[5-(4-fluoro-3-methylphenyl)-6-oxopyrimidin-1(6H)-yl]propanamide;
tert-butyl 3-[2-(2-{2-[2-(3-methylanilino)-5-{[4-(2,2,2-trifluoroethoxy)phenyl]carbamoyl}-1H-benzimidazol-1-yl]ethoxy}ethoxy)ethoxy]propanoate;
(2R)-2-{[7-bromo-2-(1-methyl-1H-pyrazol-4-yl)[1,2,4]triazolo[1,5-c]quinazolin-5-yl]amino}-1-[(2S)-2-methylmorpholin-4-yl]propan-1-one;
(2R)-2-{[7-bromo-2-(4-methoxyphenyl)[1,2,4]triazolo[1,5-c]quinazolin-5-yl]amino}-1-[(2S)-2-methylmorpholin-4-yl]propan-1-one;
(2R)-2-{[7-bromo-2-(1-methyl-1H-pyrazol-4-yl)[1,2,4]triazolo[1,5-c]quinazolin-5-yl]amino}-1-[(3R)-3,4-dimethylpiperazin-1-yl]propan-1-one;
(2R)-2-{[7-bromo-2-(4-methoxyphenyl)[1,2,4]triazolo[1,5-c]quinazolin-5-yl]amino}-1-[(3R)-3-methylmorpholin-4-yl]propan-1-one;
(2R)-2-{[7-bromo-2-(1-methyl-1H-pyrazol-4-yl)[1,2,4]triazolo[1,5-c]quinazolin-5-yl]amino}-1-[(3R)-3-methylmorpholin-4-yl]propan-1-one;
(2R)-2-{[7-bromo-2-(4-methoxyphenyl)[1,2,4]triazolo[1,5-c]quinazolin-5-yl]amino}-1-[(3R)-3,4-dimethylpiperazin-1-yl]propan-1-one;
(2R)-2-{[7-bromo-2-(4-methoxyphenyl)[1,2,4]triazolo[1,5-c]quinazolin-5-yl]amino}-1-[(1S,4S)-2-oxa-5-azabicyclo[2.2.1]heptan-5-yl]propan-1-one;
(2R)-2-{[7-bromo-2-(1-methyl-1H-pyrazol-4-yl)[1,2,4]triazolo[1,5-c]quinazolin-5-yl]amino}-1-[(1S,4S)-2-oxa-5-azabicyclo[2.2.1]heptan-5-yl]propan-1-one;
(2R)-2-{[7-bromo-2-(4-methoxyphenyl)[1,2,4]triazolo[1,5-c]quinazolin-5-yl]amino}-1-(2-oxa-6-azaspiro[3.3]heptan-6-yl)propan-1-one;
(2R)-2-{[7-bromo-2-(1-methyl-1H-pyrazol-4-yl)[1,2,4]triazolo[1,5-c]quinazolin-5-yl]amino}-1-(2-oxa-6-azaspiro[3.3]heptan-6-yl)propan-1-one;
(2R)-2-{[7-bromo-2-(4-methoxyphenyl)[1,2,4]triazolo[1,5-c]quinazolin-5-yl]amino}-1-(6-methyl-2,6-diazaspiro[3.3]heptan-2-yl)propan-1-one;
(2R)-2-{[7-bromo-2-(1-methyl-1H-pyrazol-4-yl)[1,2,4]triazolo[1,5-c]quinazolin-5-yl]amino}-1-(6-methyl-2,6-diazaspiro[3.3]heptan-2-yl)propan-1-one;
tert-butyl 6-{N-[7-bromo-2-(4-methoxyphenyl)[1,2,4]triazolo[1,5-c]quinazolin-5-yl]-D-alanyl}-2,6-diazaspiro[3.3]heptane-2-carboxylate;
tert-butyl 6-{N-[7-bromo-2-(1-methyl-1H-pyrazol-4-yl)[1,2,4]triazolo[1,5-c]quinazolin-5-yl]-D-alanyl}-2,6-diazaspiro[3.3]heptane-2-carboxylate;
(2R)-2-{[7-bromo-2-(4-methoxyphenyl)[1,2,4]triazolo[1,5-c]quinazolin-5-yl]amino}-3-methyl-1-(4-methylpiperazin-1-yl)butan-1-one;
N²-[7-bromo-2-(4-methoxyphenyl)[1,2,4]triazolo[1,5-c]quinazolin-5-yl]-N-(2-methoxyethyl)-D-valinamide;
N²-[7-bromo-2-(4-methoxyphenyl)[1,2,4]triazolo[1,5-c]quinazolin-5-yl]-N-[2-(4-methylpiperazin-1-yl)ethyl]-D-valinamide;
(2R)-2-{[7-bromo-2-(4-methoxyphenyl)[1,2,4]triazolo[1,5-c]quinazolin-5-yl]amino}-3-methyl-1-(morpholin-4-yl)butan-1-one;
(2R)-2-{[7-bromo-2-(1-methyl-1H-pyrazol-4-yl)[1,2,4]triazolo[1,5-c]quinazolin-5-yl]amino}-3-methyl-1-(morpholin-4-yl)butan-1-one;
N²-[7-bromo-2-(1-methyl-1H-pyrazol-4-yl)[1,2,4]triazolo[1,5-c]quinazolin-5-yl]-N-[2-(4-methylpiperazin-1-yl)ethyl]-D-valinamide;
(2R)-2-{[7-bromo-2-(1-methyl-1H-pyrazol-4-yl)[1,2,4]triazolo[1,5-c]quinazolin-5-yl]amino}-3-methyl-1-(4-methylpiperazin-1-yl)butan-1-one;
N²-[7-bromo-2-(1-methyl-1H-pyrazol-4-yl)[1,2,4]triazolo[1,5-c]quinazolin-5-yl]-N-(2-methoxyethyl)-D-valinamide;
2-(1-methyl-1H-pyrazol-4-yl)-5-{[(6R)-5-oxo-1,4-diazepan-6-yl]amino}[1,2,4]triazolo[1,5-c]quinazoline-7-carbonitrile;
5-{1[(6R)-5-oxo-1,4-diazepan-6-yl]amino}-2-[1-(propan-2-yl)-1H-pyrazol-4-yl][1,2,4]triazolo[1,5-c]quinazoline-7-carbonitrile;
2-(1-cyclopropyl-1H-pyrazol-4-yl)-5-{1[(6R)-5-oxo-1,4-diazepan-6-yl]amino}[1,2,4]triazolo[1,5-c]quinazoline-7-carbonitrile;
2-(1-cyclobutyl-1H-pyrazol-4-yl)-5-{[(6R)-5-oxo-1,4-diazepan-6-yl]amino}[1,2,4]triazolo[1,5-c]quinazoline-7-carbonitrile;
2-[1-(difluoromethyl)-1H-pyrazol-4-yl]-5-{1[(6R)-5-oxo-1,4-diazepan-6-yl]amino}[1,2,4]triazolo[1,5-c]quinazoline-7-carbonitrile;
5-{1[(6R)-5-oxo-1,4-diazepan-6-yl]amino}-2-(1H-pyrazol-4-yl)[1,2,4]triazolo[1,5-c]quinazoline-7-carbonitrile;
(6R)-6-{[7-(methanesulfonyl)-2-(4-methoxyphenyl)[1,2,4]triazolo[1,5-c]quinazolin-5-yl]amino}-1,4-diazepan-5-one;
(6R)-6-{[7-(methanesulfonyl)-2-(1-methyl-1H-pyrazol-4-yl)[1,2,4]triazolo[1,5-c]quinazolin-5-yl]amino}-1,4-diazepan-5-one;
(6R)-6-{[7-(ethylsulfanyl)-2-(1-methyl-1H-pyrazol-4-yl)[1,2,4]triazolo[1,5-c]quinazolin-5-yl]amino}-1,4-diazepan-5-one;
(6R)-6-{[7-(ethylsulfanyl)-2-(4-methoxyphenyl)[1,2,4]triazolo[1,5-c]quinazolin-5-yl]amino}-1,4-diazepan-5-one;
(6R)-6-{[7-(ethanesulfonyl)-2-(4-methoxyphenyl)[1,2,4]triazolo[1,5-c]quinazolin-5-yl]amino}-1,4-diazepan-5-one;
(6R)-6-{[2-(4-methoxyphenyl)-7-(propane-2-sulfonyl)[1,2,4]triazolo[1,5-c]quinazolin-5-yl]amino}-1,4-diazepan-5-one;
(6R)-6-{[7-(ethanesulfonyl)-2-(1-methyl-1H-pyrazol-4-yl)[1,2,4]triazolo[1,5-c]quinazolin-5-yl]amino}-1,4-diazepan-5-one;
(6R)-6-{[2-(1-methyl-1H-pyrazol-4-yl)-7-(propane-2-sulfonyl)[1,2,4]triazolo[1,5-c]quinazolin-5-yl]amino}-1,4-diazepan-5-one;
(6R)-6-({7-(methanesulfonyl)-2-[1-(propan-2-yl)-1H-pyrazol-4-yl][1,2,4]triazolo[1,5-c]quinazolin-5-yl}amino)-1,4-diazepan-5-one;
(6R)-6-{[2-(1-ethyl-3-methyl-1H-pyrazol-4-yl)-7-(methanesulfonyl)[1,2,4]triazolo[1,5-c]quinazolin-5-yl]amino}-1,4-diazepan-5-one;
(6R)-6-{[2-(1-methyl-1H-pyrazol-5-yl)-7-(trifluoromethyl)[1,2,4]triazolo[1,5-c]quinazolin-5-yl]amino}-1,4-diazepan-5-one;
(6R)-6-{[2-(4-chlorophenyl)-7-(trifluoromethyl)[1,2,4]triazolo[1,5-c]quinazolin-5-yl]amino}-1,4-diazepan-5-one;
(6S)-6-{[2-(1,3-dimethyl-1H-pyrazol-4-yl)-7-(trifluoromethyl)[1,2,4]triazolo[1,5-c]quinazolin-5-yl]amino}-1,4-diazepan-5-one;
(6S)-6-{[2-(1-ethyl-3-methyl-1H-pyrazol-4-yl)-7-(trifluoromethyl)[1,2,4]triazolo[1,5-c]quinazolin-5-yl]amino}-1,4-diazepan-5-one;
(6S)-6-{[2-(1-cyclopropyl-1H-pyrazol-4-yl)-7-(trifluoromethyl)[1,2,4]triazolo[1,5-c]quinazolin-5-yl]amino}-1,4-diazepan-5-one;
(6R)-6-{[7-chloro-2-(1,3-dimethyl-1H-pyrazol-4-yl)[1,2,4]triazolo[1,5-c]quinazolin-5-yl]amino}-1,4-diazepan-5-one;
N-{[3-(3,4-dimethylphenyl)-4,5-dihydro-1,2-oxazol-5-yl]methyl}methanesulfonamide;
3-(2-amino-7-oxo-7,8-dihydropyrido[4,3-d]pyrimidin-6(5H)-yl)-N-[4-fluoro-3-(trifluoromethoxy)phenyl]-4-methylbenzamide;
(6R)-6-({7-cyclopropyl-2-[1-(cyclopropylmethyl)-1H-pyrazol-4-yl][1,2,4]triazolo[1,5-c]quinazolin-5-yl}amino)-1,4-diazepan-5-one;
(6R)-6-({7-cyclopropyl-2-[1-(propan-2-yl)-1H-pyrazol-4-yl][1,2,4]triazolo[1,5-c]quinazolin-5-yl}amino)-1,4-diazepan-5-one;
(6R)-6-({7-chloro-2-[1-(propan-2-yl)-1H-pyrazol-4-yl][1,2,4]triazolo[1,5-c]quinazolin-5-yl}amino)-1,4-diazepan-5-one;
(6R)-6-{[7-chloro-2-(1-ethyl-1H-pyrazol-4-yl)[1,2,4]triazolo[1,5-c]quinazolin-5-yl]amino}-1,4-diazepan-5-one;
(6R)-6-{[7-chloro-2-(1-ethyl-3-methyl-1H-pyrazol-4-yl)[1,2,4]triazolo[1,5-c]quinazolin-5-yl]amino}-1,4-diazepan-5-one;
(6S)-6-{[7-cyclopropyl-2-(4-methoxyphenyl)[1,2,4]triazolo[1,5-c]quinazolin-5-yl]amino}-1,4-diazepan-5-one;
(6S)-6-({2-[1-(propan-2-yl)-1H-pyrazol-4-yl]-7-(trifluoromethyl)[1,2,4]triazolo[1,5-c]quinazolin-5-yl}amino)-1,4-diazepan-5-one;
N-(2-oxo-2,3-dihydro-1H-indol-5-yl)-3-(2-phenyl-1,3-thiazol-4-yl)benzamide;
(6R)-6-{[7-bromo-2-(1,3-dimethyl-1H-pyrazol-4-yl)[1,2,4]triazolo[1,5-c]quinazolin-5-yl]amino}-1,4-diazepan-5-one;
(6R)-6-{[2-(1-cyclopropyl-1H-pyrazol-4-yl)-7-(trifluoromethyl)[1,2,4]triazolo[1,5-c]quinazolin-5-yl]amino}-1,4-diazepan-5-one;
(6S)-6-{[7-chloro-2-(4-fluorophenyl)[1,2,4]triazolo[1,5-c]quinazolin-5-yl]amino}-1,4-diazepan-5-one;
(6S)-6-{[2-(4-fluorophenyl)[1,2,4]triazolo[1,5-c]quinazolin-5-yl]amino}-1,4-diazepan-5-one;
(6R)-6-{[7-chloro-2-(1-cyclopropyl-1H-pyrazol-4-yl)[1,2,4]triazolo[1,5-c]quinazolin-5-yl]amino}-1,4-diazepan-5-one;
(6S)-6-{[2-(1-methyl-1H-pyrazol-3-yl)[1,2,4]triazolo[1,5-c]quinazolin-5-yl]amino}-1,4-diazepan-5-one;
(6R)-6-({7-bromo-2-[1-(cyclopropylmethyl)-1H-pyrazol-4-yl][1,2,4]triazolo[1,5-c]quinazolin-5-yl}amino)-1,4-diazepan-5-one;
(6R)-6-{[2-(1,5-dimethyl-1H-pyrazol-4-yl)-7-fluoro[1,2,4]triazolo[1,5-c]quinazolin-5-yl]amino}-1,4-diazepan-5-one;
(6S)-6-{[2-(4-methoxyphenyl)-7-methyl[1,2,4]triazolo[1,5-c]quinazolin-5-yl]amino}-1,4-diazepan-5-one;
(6R)-6-({7-fluoro-2-[1-(propan-2-yl)-1H-pyrazol-4-yl][1,2,4]triazolo[1,5-c]quinazolin-5-yl}amino)-1,4-diazepan-5-one;
(6S)-6-{[7-bromo-2-(4-methoxyphenyl)[1,2,4]triazolo[1,5-c]quinazolin-5-yl]amino}-1,4-diazepan-5-one;
(6S)-6-{[7-bromo-2-(4-fluorophenyl)[1,2,4]triazolo[1,5-c]quinazolin-5-yl]amino}-1,4-diazepan-5-one;
(6R)-6-{[7-bromo-2-(1-ethyl-3-methyl-1H-pyrazol-4-yl)[1,2,4]triazolo[1,5-c]quinazolin-5-yl]amino}-1,4-diazepan-5-one;
(6R)-6-{[2-(1,3-dimethyl-1H-pyrazol-4-yl)-7-(trifluoromethyl)[1,2,4]triazolo[1,5-c]quinazolin-5-yl]amino}-1,4-diazepan-5-one;
(6R)-6-{[7-bromo-2-(1H-pyrazol-4-yl)[1,2,4]triazolo[1,5-c]quinazolin-5-yl]amino}-1,4-diazepan-5-one;
(6R)-6-{[7-bromo-2-(4-chlorophenyl)[1,2,4]triazolo[1,5-c]quinazolin-5-yl]amino}-1,4-diazepan-5-one;
(6R)-6-({7-bromo-2-[1-(difluoromethyl)-1H-pyrazol-4-yl][1,2,4]triazolo[1,5-c]quinazolin-5-yl}amino)-1,4-diazepan-5-one;
(6R)-6-{[7-bromo-2-(1,5-dimethyl-1H-pyrazol-4-yl)[1,2,4]triazolo[1,5-c]quinazolin-5-yl]amino}-1,4-diazepan-5-one;
(6R)-6-{[7-bromo-2-(1-ethyl-1H-pyrazol-4-yl)[1,2,4]triazolo[1,5-c]quinazolin-5-yl]amino}-1,4-diazepan-5-one;
(6R)-6-({7-bromo-2-[1-(propan-2-yl)-1H-pyrazol-4-yl][1,2,4]triazolo[1,5-c]quinazolin-5-yl}amino)-1,4-diazepan-5-one;
(6R)-6-({7-bromo-2-[1-(cyclobutylmethyl)-1H-pyrazol-4-yl][1,2,4]triazolo[1,5-c]quinazolin-5-yl}amino)-1,4-diazepan-5-one;
(6S)-6-{[7-bromo-2-(1-methyl-1H-pyrazol-4-yl)[1,2,4]triazolo[1,5-c]quinazolin-5-yl]amino}-1,4-diazepan-5-one;
(6S)-6-({7-bromo-2-[1-(propan-2-yl)-1H-pyrazol-4-yl][1,2,4]triazolo[1,5-c]quinazolin-5-yl}amino)-1,4-diazepan-5-one;
(2R)-2-{[7-bromo-2-(1-methyl-1H-pyrazol-4-yl)[1,2,4]triazolo[1,5-c]quinazolin-5-yl]amino}-1-(piperazin-1-yl)propan-1-one hydrogen chloride (1/1);
(2R)-2-{[7-bromo-2-(4-methoxyphenyl)[1,2,4]triazolo[1,5-c]quinazolin-5-yl]amino}-1-(piperazin-1-yl)propan-1-one;
N²-[7-bromo-2-(4-methoxyphenyl)[1,2,4]triazolo[1,5-c]quinazolin-5-yl]-N-[2-(methylamino)ethyl]-D-alaninamide hydrogen chloride (1/1);
(2R)-2-{[2-(4-methoxyphenyl)[1,2,4]triazolo[1,5-c]quinazolin-5-yl]amino}-1-(piperazin-1-yl)propan-1-one hydrogen chloride (1/1);
N²-[7-bromo-2-(1-methyl-1H-pyrazol-4-yl)[1,2,4]triazolo[1,5-c]quinazolin-5-yl]-N-[2-(methylamino)ethyl]-D-alaninamide;
N²-[2-(4-methoxyphenyl)[1,2,4]triazolo[1,5-c]quinazolin-5-yl]-N-[2-(methylamino)ethyl]-D-alaninamide hydrogen chloride (1/1);
N²-[7-bromo-2-(1-methyl-1H-pyrazol-4-yl)[1,2,4]triazolo[1,5-c]quinazolin-5-yl]-N-[2-(methylamino)ethyl]-D-alaninamide hydrogen chloride (1/1);
N²-[7-bromo-2-(4-methoxyphenyl)[1,2,4]triazolo[1,5-c]quinazolin-5-yl]-N-[2-(piperazin-1-yl)ethyl]-D-alaninamide hydrogen chloride (1/2);
N²-[7-bromo-2-(1-methyl-1H-pyrazol-4-yl)[1,2,4]triazolo[1,5-c]quinazolin-5-yl]-N-[2-(piperazin-1-yl)ethyl]-D-alaninamide hydrogen chloride (1/2);
(6R)-6-{[7-bromo-2-(1-methyl-1H-pyrazol-4-yl)[1,2,4]triazolo[1,5-c]quinazolin-5-yl]amino}-1-methyl-1,4-diazepan-5-one;
(6R)-6-{[7-bromo-2-(4-methoxyphenyl)[1,2,4]triazolo[1,5-c]quinazolin-5-yl]amino}1-methyl-1,4-diazepan-5-one;
(3R)-3-{[7-(3-methoxy-3-methylbutoxy)-2-(4-methoxyphenyl)[1,2,4]triazolo[1,5-c]quinazolin-5-yl]amino}azepan-2-one;
(3R)-3-{[7-(2-hydroxy-2-methylpropoxy)-2-(4-methoxyphenyl)[1,2,4]triazolo[1,5-c]quinazolin-5-yl]amino}azepan-2-one;
(3R)-3-{[7-(cyclobutyloxy)-2-(4-methoxyphenyl)[1,2,4]triazolo[1,5-c]quinazolin-5-yl]amino}azepan-2-one;
(3R)-3-{[7-(cyclopropyloxy)-2-(4-methoxyphenyl)[1,2,4]triazolo[1,5-c]quinazolin-5-yl]amino}azepan-2-one;
(3R)-3-{[7-(3-hydroxy-3-methylbutoxy)-2-(4-methoxyphenyl)[1,2,4]triazolo[1,5-c]quinazolin-5-yl]amino}azepan-2-one;
(3R)-3-{[2-(4-methoxyphenyl)-7-(2-oxopyrrolidin-1-yl)[1,2,4]triazolo[1,5-c]quinazolin-5-yl]amino}azepan-2-one;
(3R)-3-{[2-(4-methoxyphenyl)-7-(oxetan-3-yl)[1,2,4]triazolo[1,5-c]quinazolin-5-yl]amino}azepan-2-one;
(3R)-3-{[7-(methoxymethyl)-2-(4-methoxyphenyl)[1,2,4]triazolo[1,5-c]quinazolin-5-yl]amino}azepan-2-one, and
(3R)-3-{[7-(methoxymethyl)-2-(1-methyl-1H-pyrazol-4-yl)[1,2,4]triazolo[1,5-c]quinazolin-5-yl]amino}azepan-2-one,
their polymorphs, enantiomeres, diastereomeres, racemates, tautomeres, N-oxides, hydrates and solvates, as well as their physiological acceptable salts and solvates of these salts, as well as mixtures of the same.

4: A method of preparing a compound of general formula (I) according to claim 1, said method comprising the step of allowing an intermediate compound of general formula (V): 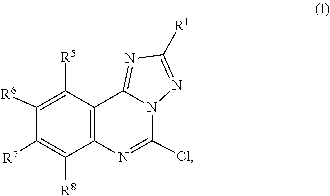
in which R¹, R⁵, R⁶, R⁷ and R⁸ are as defined for the compound of general formula (I),
to react with a compound of general formula (VII): 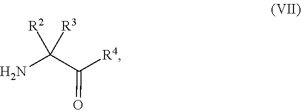
in which R², R³ and R⁴ are as defined for the compound of general formula (I),
thereby giving a compound of general formula (I):

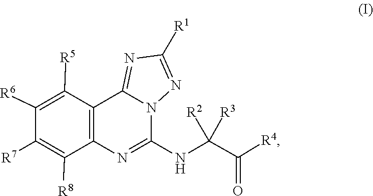

5. (canceled)

6: A pharmaceutical composition comprising a compound of general formula (I) according to claim 1 and one or more pharmaceutically acceptable excipients.

7: A pharmaceutical combination comprising:
(i) one or more compounds of general formula (I) according to claim 1; and
(ii) one or more pharmaceutical active anti cancer compounds, or
one or more pharmaceutical active immune checkpoint inhibitors.

8-10. (canceled)

11: A compound of general formula (V): 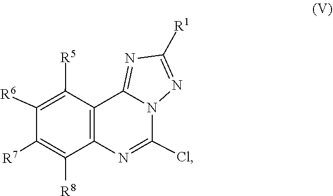
in which R¹, R⁵, R⁶, R⁷ and R⁸ are as defined for the compound of general formula (I) according to claim 1.

12. (canceled)

13: A method of treatment or prophylaxis of a disease comprising administering to a subject in need thereof a therapeutically effective amount of the compound of formula (I) of claim 1.

14: The method of claim 13, wherein the disease is cancer or a condition with dysregulated immune response or other disorder associate with aberrant AHR signaling.

15: The method of claim 13, wherein the disease is a liquid or a solid tumor.
